(19) United States
(12) Patent Application Publication
(10) Pub. No.: US 2012/0094935 A1
(43) Pub. Date: Apr. 19, 2012

(54) METHODS FOR CREATING OR IDENTIFYING COMPOUNDS THAT BIND TUMOR NECROSIS FACTOR ALPHA

(76) Inventors: Anthony P. West, Pasadena, CA (US); Zhiru Yang, Bejing (CN); Pamela J. Bjorkman, La Canada, CA (US)

(21) Appl. No.: 13/263,308

(22) PCT Filed: Apr. 9, 2010
(86) PCT No.: PCT/US2010/030649

§ 371 Date: Dec. 20, 2011

Related U.S. Application Data
(60) Provisional application No. 61/168,133, filed on Apr. 9, 2009.

(51) Int. Cl. A61K 38/16; C12P 21/02; G01N 33/566; C07K 14/065; G06F 19/00; C12N 15/63; C07K 14/525; C12N 5/10; A61P 29/00; A61P 37/00; C07K 1/107; C12N 15/39

(52) U.S. Cl. 514/21.2; 530/402; 435/69.1; 436/501; 530/350; 536/23.72; 435/320.1; 530/409; 435/348; 702/19

(57) ABSTRACT

Methods for creating therapeutic candidate compounds or of identifying therapeutic candidate compounds for use in treating TNFα-mediated diseases or conditions are described that utilize parameters, including amino acid residues, obtained by analysis of a Yatapoxvirus 2L-TNFα crystal structure.

### CROSS-REFERENCE TO RELATED APPLICATION(S)

This application claims priority to and the benefit of U.S. Provisional Application Ser. No. 61/168,133, filed Apr. 9, 2009, and titled “Crystal Structure of Poxvirus Tumor Necrosis Factor-alpha (TNFα)-Binding Protein 2L in Complex with TNFα, Useful for the Design and Development of Anti-Inflammatory Pharmaceuticals,” the entire content of which is incorporated herein by reference.

### INCORPORATION BY REFERENCE

The material in the text file titled “65251SequenceListing”, created Apr. 9, 2010, and being 2700 bytes, is herein incorporated by reference in its entirety.

### FIELD

The present invention relates to methods of creating and/or identifying thereapeutic candidate compounds expected to have useful immonomodulatory and/or anti-inflammatory properties. In particular, the methods make use of a crystal structure of a Yatapoxvirus 2L polypeptide bound to human TNFα to provide guidance as to desirable characteristics of the therapeutic candidate compounds.

### BACKGROUND

Tumor necrosis factor (TNF) secreted primarily by macrophages and monocytes, is a potent mediator cytokine of inflammation and the immune response to various pathogens (Aggarwal, B B 2003, Nat. Rev. Immunol. 3:745-756; Ware, C. F., 2005, Annu. Rev. Immunol. 23:787-819). The proinflammatory cytokine tumor necrosis factor α (TNFa) is the prototype member of the TNF superfamily that forms trimers, and is expressed as a membrane-bound 26-kDa protein and a soluble 17-kDa protein, both of which are biologically active. TNFα is a major target for pharmaceutical intervention in a number of autoimmune diseases including rheumatoid arthritis, septic shock, ulcerative colitis, Crohn's disease and other syndromes (Mocellin et al., 2005, Cytokine Growth Ractor Rev., 16:35-53).

Large DNA viruses such as poxviruses, which replicate exclusively within the cytoplasm of infected cells, encode diverse immunomodulatory proteins to modulate, avoid, or diminish multiple antiviral responses mediated by hosts (Seet et al., 2003, Annu. Rev. Immunol., 21:377-423; Alcami, 2003, Nat. Rev. Immunol. 3:36-50; Nazarian et al., 2006, Future Virol., 1:123-132). Given the broad immune effects of TNFα, viruses have developed potent TNFα inhibitors—some derived from host TNF receptors (Rahman, 2006, PLoS Pathog, 2 e4). The Yatapoxvirus genus of poxviruses includes Tanapox virus (TPV), Yaba monkey tumor virus (YMTV), and Yaba-like disease virus (YLDV) (Knight et al., 1989, Virology, 172:116-124). Several members of this genus of poxviruses have been found to encode an hTNFα inhibitor, designated as 2L, that bears little homology to endogenous hTNF receptors.

The 2L poxvirus protein was originally isolated as a potential human therapeutic, with the rationale that agents developed over millennia in the struggle between viruses and the vertebrate immune system may be particularly well tuned for modulation of the immune response. However, since even the existing anti-TNFα drugs exhibit limitations in efficacy for treating disease, there is a need for improved human anti-TNFα therapeutics based on the 2L poxvirus protein and methods for obtaining such therapeutics.

### SUMMARY

According to an aspect of the invention, there is provided a method of creating a therapeutic candidate compound comprising: providing a secreted form of a 2L polypeptide of SEQ ID No. 1, SEQ ID No. 2, or SEQ ID No. 3; modifying at least one amino acid of the 2L polypeptide at a position other than the 2L interface amino acids HIS58, GLU99, GLY101, PHE104, LYS125, GLY157, TYR160, MET161, GLY162, LEU165, LEU168, GLN171, LYS175, GLU225, VAL229, ASN230, GLY231, ARG232, or GLU289 for SEQ ID No.'s 1-2 or HIS57, GLU100, GLY102, PHE105, LYS126, GLY157, TYR161, ALA162, GLY163, LEU166, PHE169, GLN172, LYS176, GLU226, VAL230, ASN231, GLY232, ARG233, or GLU290 for SEQ ID No. 3, to form a candidate compound; and, purifying the candidate compound.

According to another aspect of the invention, there is provided a method of creating a therapeutic candidate compound comprising: providing a sequence of a 2L polypeptide comprising SEQ ID No. 1, SEQ ID No. 2, or SEQ ID No. 3; modifying at least one amino acid of the sequence of the 2L polypeptide at a position other than HIS58, GLU99, GLY101, PHE104, LYS125, GLY157, TYR160, MET161, GLY162, LEU165, LEU168, GLN171, LYS175, GLU225, VAL229, ASN230, GLY231, ARG232, or GLU289 for SEQ ID No.'s 1-2 or at a position other than HIS57, GLU100, GLY102, PHE105, LYS126, GLY157, TYR161, ALA162, GLY163, LEU166, PHE169, GLN172, LYS176, GLU226, VAL230, ASN231, GLY232, ARG233, or GLU290 for SEQ ID No. 3, to form a sequence of a candidate compound; expressing the sequence of the candidate compound from a host cell to create a secreted form of the candidate compound; and, purifying the candidate compound.

These methods may further comprise verifying that the candidate compound binds to human TNFα with a binding affinity Kd of from about 40 picomoles to about 440 picomoles.

According to yet another aspect of the invention, there is provided a method of creating a therapeutic candidate compound comprising: providing a secreted form of a 2L polypeptide of SEQ ID No. 1, SEQ ID No. 2, or SEQ ID No. 3; modifying at least one amino acid of the 2L polypeptide at a position selected from the group consisting of HIS58, GLU99, GLY101, PHE104, LYS125, GLY157, TYR160, MET161, GLY162, LEU165, LEU168, GLN171, LYS175, GLU225, VAL229, ASN230, GLY231, ARG232, and GLU289 for SEQ ID No.'s 1-2 or at a position selected from the group consisting of HIS57, GLU100, GLY102, PHE105, LYS126, GLY157, TYR161, ALA162, GLY163, LEU166, PHE169, GLN172, LYS176, GLU226, VAL230, ASN231, GLY232, ARG233, and GLU290 for SEQ ID No. 3, to form a candidate compound; and, purifying the candidate compound.

According to still another aspect of the invention, there is provided a method of creating a therapeutic candidate compound comprising: providing a sequence of a 2L polypeptide comprising SEQ ID No. 1, SEQ ID No. 2, or SEQ ID No. 3; modifying at least one amino acid of the sequence of the 2L polypeptide at a position selected from the group consisting of HIS58, GLU99, GLY101, PHE104, LYS125, GLY157, TYR160, MET161, GLY162, LEU165, LEU168, GLN171, LYS175, GLU225, VAL229, ASN230, GLY231, ARG232, and GLU289 for SEQ ID No.'s 1-2 or at a position selected from the group consisting of HIS57, GLU100, GLY102, PHE105, LYS126, GLY157, TYR161, ALA162, GLY163, LEU166, PHE169, GLN172, LYS176, GLU226, VAL230, ASN231, GLY232, ARG233, and GLU290 for SEQ ID No. 3, to form a sequence of a candidate compound; expressing the sequence of the candidate compound from a host cell to create a secreted form of the candidate compound; and, purifying the candidate compound.

These methods may further comprise verifying that the candidate compound binds to human TNFα with a binding affinity K_{d} less than that of the 2L polypeptide.

These methods may further comprise an N-terminal signal sequence.

These methods may be provided wherein the immature sequence is according to SEQ ID No. 15, SEQ ID No. 16, or SEQ ID NO. 17.

The foregoing methods may further comprise confirming in vitro that the candidate compound suppresses signaling via the TNFα cascade. The methods may further comprise confirming in vitro that the candidate compound competitively inhibits human TNFR1 or human TNFR2 from binding to human TNFα. The modification of the at least one amino acid may be selected from the group consisting of substitution, addition and deletion.

According to even another aspect of the present invention, there is provided a method for determining whether or not a test compound is a therapeutic candidate compound for use in treating a TNFα mediated disease or condition in humans, the method comprising: providing the test compound or an electronic representation of the test compound; determining whether the test compound or electronic representation of the test compound binds to at least TNFα interface amino acids TYR87, ARG31, ARG32, ASN30, SER86, ARG82, GLU127, ASN34, VAL91, ALA33, VAL91, GLN21, ARG44, GLU42, LEU37, LEU43, VAL41 and GLN27 of hTNFα, or competitively inhibits binding of hTNFR1 or hTNFR2 to the TNFα interface amino acids, to within a specified confidence interval; for test compounds that bind to the TNFα interface amino acids or competitively inhibit binding of human TNFR1 or human TNFR2 to the TNFα interface amino acids, confirming in vitro that the test compound suppresses signaling via the TNFα cascade, thereby rendering it a therapeutic candidate compound.

In any of the foregoing methods, the method may further comprise verifying that the candidate compound binds to human TNFα with a shape complementarity index of from 0.64 to 0.78. The methods may further comprise verifying that the candidate compound, upon binding to human TNFα, imparts a change in conformation of human TNFα at GLN21, ALA22, GLU23, GLY24, GLN25, ARG31, ARG32, ALA33, ASN34, ALA35, PHE144, ALA145, GLU146, SER147, and GLY148. The methods may further comprise verifying that the modification reduces immunogenicity of the candidate compound as compared with the 2L polypeptide of SEQ ID No.'s 1, 2 or 3. The methods may further comprise verifying that the modification alters the circulating half life of the candidate compound as compared with the 2L polypeptide of SEQ ID No.'s 1, 2 or 3. The methods may further comprise verifying that the candidate compound binds to human TNFα over a surface area of at least 2000 Å².

According to yet still another aspect of the invention, there is provided a therapeutic candidate compound obtained according to any of the foregoing methods.

According to yet even another aspect of the invention, there is provided a method of treating a TNFα mediated disease or condition in humans comprising administering a therapeutic candidate compound obtained according to any of the foregoing methods in an amount and for a time sufficient to achieve a therapeutic effect. This can be alternatively described as use of a therapeutic candidate compound according to any of the foregoing methods in an amount and for a time sufficient to achieve a therapeutic effect in treating a TNFα mediated disease or condition in humans. It is contemplated to use the therapeutic candidate compound in the manufacture of a medicament for treating a TNFα mediated disease or condition or in a pharmaceutical composition comprising the purified therapeutic candidate compound with a pharmaceutically acceptable diluent formulated for administration to a patient suffering from or at risk of developing a TNFα mediated disease or condition.

A purified nucleic acid molecule that encodes a polypeptide comprising the sequence of the candidate compound is also provided as part of this invention, along with any purified nucleic acid molecule that hybridizes under high stringency conditions to the aforementioned nucleic acid molecule. A vector comprising the nucleic acid molecule and a host cell comprising the vector are also provided.

According to yet even still another aspect of the invention, there is provided a crystal of the secreted form of Yaba-like disease virus 2L protein bound to human TNFα. The Yaba-like disease virus 2L protein may comprise an amino acid sequence according to SEQ ID No. 1. The crystal may have a three-dimensional structure as defined by the atomic coordinates of Table 4 with a root mean square deviation of 1.0 Å for each atomic coordinate.

### BRIEF DESCRIPTION OF THE DRAWINGS

These and other features and advantages of the present invention will be better understood by reference to the following detailed description when considered in conjunction with the attached drawings, in which:

FIG. 1A is a ribbon diagram of the 2L-TNFα complex looking down the 3-fold symmetry axis;

FIG. 1B is a ribbon diagram of the 2L-TNFα complex with the 3-fold axis vertical;

FIG. 1C shows a surface representation of TNFα from the complex structure with the 2L binding site in the darkened region;

FIG. 2A shows a ribbon diagram of 2L with the α3 domain insertion;

FIG. 2B shows stereo Ca superposition of the α1-α2 platforms of 2L with HLA-A2 and FcRn;

FIG. 2C shows a surface representation of the 2L α1-α2 platform shown in the same orientation as FIG. 2B;

FIG. 2D shows a close-up of a ribbon diagram of the interface between the 2L α1-α2 platform and α3 domain with the interface amino acids labeled;

FIG. 2E shows a stereo superposition of 2L and HLA-A2 with an arrow indicating displacement of the 2L α3 domain compared to the HLA-A2 α3 domain;

FIG. 2F shows stereo representation of 2L with β2m showing N-linked glycosylation at Asn6;

FIG. 3A shows surface representations of 2L (left) and the 2L-TNFα complex (right);

FIG. 3B shows ribbon diagrams corresponding to the surface representations of FIG. 3A;

FIG. 3C shows stereo close-ups of the 2L-TNFα interface with the α3 domain insertion;

FIG. 4 shows a sequence alignment of mature poxvirus 2L proteins including YLDV (Yaba-like disease virus); TPV (Tanapox virus); YMTV (Yaba monkey tumor virus), Deerpox (Deerpox virus strain W-848-83; Swinepox (Swinepox open reading frame SPV003) with boxes highlighting the interface residues;

FIG. 5 shows a sequence alignment of mammalian TNFα proteins and human TNFβ; and,

FIG. 6 shows a stereo superposition of 2L-bound TNFα and unbound TNFα.

### DETAILED DESCRIPTION

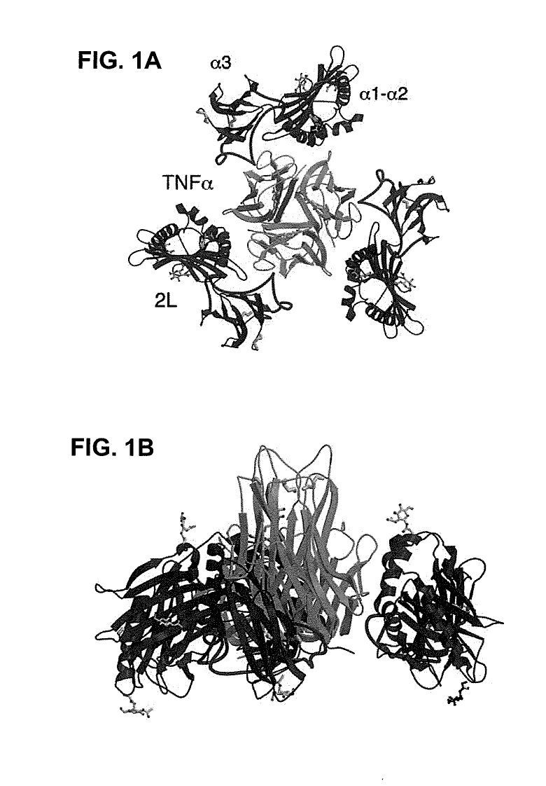

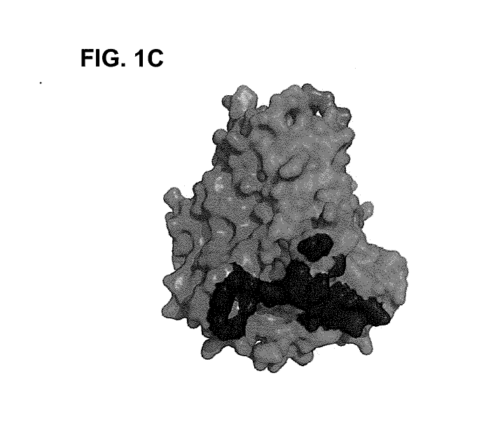

Design and development of a therapeutic agent that binds TNFα while evading the immune system can be efficiently realized from the specific binding interface of the poxvirus 2L protein and TNFα. As shown herein, the Yaba-like disease virus (YLDV) 2L protein complexed to TNFα has been isolated and crystallized, and its crystal structure has been determined and analyzed. The crystal structure data includes the three-dimensional molecular and atomic coordinates (Table 4) and computed modeling of the 2L-TNFα complex (FIG. 1A-1C). In addition to the disclosed crystal structure, the present invention also provides methods for identifying and designing novel 2L-based TNFα binding compounds that modulate TNFα activity.

U.S. Pat. Nos. 7,585,507 and 6,894,155 to McFadden et al., are incorporated by reference herein in their entirety. These patents describe immune modulation of TNFα using full length (exclusive of signal sequence) 2L molecules. As described herein, a method is provided for designing candidate TNFα-binding compounds based on the specific 2L and TNFα residues found to interact at the 2L-TNFα interface.

### DEFINITIONS

Unless defined otherwise, all technical and scientific terms used herein have the meaning commonly understood by one of ordinary skill in the art relevant to the invention. The definitions below supplement those in the art and are directed to the embodiments described in the current application.

Immature YLDV, TPV and YMTV 2L proteins are represented by SEQ ID NOs. 15, 16 and 17, respectively. Mature untagged YLDV, TPV and YMTV 2L proteins are represented by SEQ ID NOs: 1, 2 and 3, respectively.

As used herein, the “2L-TNFα interface” refers to the amino acid residues of the 2L protein that interact directly with TNFα, and the TNFα amino acid residues that interact directly with 2L. The interface residues are listed in Table 1. Direct interaction is defined by those residues that have at least 2% ASA (accessible surface area) and/or form a salt bridge or hydrogen bond. As used herein, the interface can refer to both the 2L and TNFα amino acids that interact with each other.

The “2L interface” and the “2L interface residues” refer to the 2L amino acids that directly interact with TNFα. The 2L interface residues of YLDV 2L (SEQ ID NO: 1) and TPV 2L (SEQ ID NO: 2) include: HIS58, GLU99, GLY101, PHE104, LYS125, GLY157, TYR160, MET161 or ALA161, GLY162, LEU165, LEU168 or PHE168, GLN171, LYS175, GLU225, VAL229, ASN230, GLY231, ARG232, and GLU289. The 2L interface residues of YMTV 2L (SEQ ID NO: 3) include: HIS57, GLU100, GLY102, PHE105, LYS126, GLY157, TYR161, ALA162, GLY163, LEU166, PHE169, GLN172, LYS176, GLU226, VAL230, ASN231, GLY232, ARG233, and GLU190.

The “TNFα interface” and the “TNFα interface residues” refer to the human TNFα amino acids that directly interact with 2L. The TNFα interface residues include: TYR87, ARG31, ARG32, ASN30, SER86, ARG82, GLU127, ASN34, VAL91, ALA33, VAL91, GLN21, ARG44, GLU42, LEU37, LEU43, VAL41, and GLN27.

“Polypeptide,” “polypeptide fragment,” “peptide,” or “peptide fragment” means a chain of two or more amino acids, regardless of any post-translational modification (e.g., glycosylation, acetylation, or phosphorylation), constituting all or part of a naturally or non-naturally occurring polypeptide. A “protein” can be made up of one or more polypeptides or one or more peptides.

“Post-translational modification” means any change to a polypeptide or polypeptide fragment during or after synthesis. Post-translational modifications can be produced naturally (such as during synthesis within a cell) or generated artificially (such as by recombinant or chemical means). One example of a post-translational modification occurs during secretion from a cell, in which an N-terminal signal sequence is cleaved from an immature intracellular protein to yield a extracellular mature, processed, or secreted form of the protein. The amino acids that make up the N-terminal signal sequence of a protein can be readily determined by persons skilled in the art from the immature protein sequence using, for example, software tools such as SignalP (as originally described by Henrik Nielsen, Jacob Engelbrecht, Søren Brunak and Gunnar von Heijne in “Identification of prokaryotic and eukaryotic signal peptides and prediction of their cleavage sites.”, Protein Engineering, 10:1-6, 1997, and available for use at www.cbs.dtu.dk/Services/SignalP)

By “candidate compound” or “compound” is meant a molecule, be it naturally-occurring or artificially-derived, that is assayed for its ability to bind TNFα by employing one of the assay methods described herein. Candidate compounds may include, for example, peptides, polypeptides, aptamers, Fc fusion proteins, antibodies, synthesized organic molecules, naturally occurring organic molecules, nucleic acid molecules, and biologically active fragments thereof.

A “mutation” includes a deletion, a conserved amino acid change and/or non-conserved amino acid change, or an addition of an amino acid.

By “analog” is meant any substitution, addition, or deletion in the amino acid sequence of a 2L-derived TNFα-binding candidate compound that exhibits properties that are at least 50%, preferably at least 75%, more preferably at least 90%, and most preferably at least 95% of the immunomodulatory properties of the poxvirus 2L protein from which it is derived. Analogs can differ from the naturally occurring 2L protein by amino acid sequence differences, by amino acid sequence or post-translational modifications, or by both. Modifications include in vivo and in vitro chemical derivatization of polypeptides, e.g., acetylation, carboxylation, phosphorylation, or glycosylation. Such modifications may occur during polypeptide synthesis or processing or following treatment with isolated modifying enzymes. Analogs can also differ from the naturally occurring poxvirus 2L polypeptide by alterations in primary sequence. These include genetic variants, both natural and induced (for example, resulting from random mutagenesis by irradiation or exposure to agents such as ethanemethylsulfate, or by site-specific mutagenesis as described in Sambrook, Fritsch and Maniatis, Molecular Cloning: A Laboratory Manual (2d ed.), CSH Press, 1989, hereby incorporated by reference; or Ausubel et al., Current Protocols in Molecular Biology, John Wiley & Sons, New York, 1994, hereby incorporated by reference). Also included are polypeptide molecules that contain residue substitutions for L-amino acids, e.g., D-amino acids or non-naturally occurring synthetic amino acids. Fragments and analogs can be generated using standard techniques, for example, solid phase peptide synthesis or polymerase chain reaction (PCR).

“2L-derived TNFα-binding candidate nucleic acid molecule” means a nucleic acid molecule, such as a genomic DNA, cDNA, or RNA (e.g., mRNA) molecule that encodes a 2L-derived TNFα-binding candidate polypeptide having the desired characteristics or biological activities of a poxvirus 2L polypeptide as described herein.

“Immune function” or “immunoreactivity” refers to the ability of the immune system or immune system factors in an in vitro assay, to respond to a foreign antigen as measured by standard assays. “Modulate” or “modulating” means the induction of a quantitative change, either by decrease or increase, in the response of a target cell, sample, or organism, as a result of an interaction with a TNFα protein or nucleic acid molecule or candidate compound. The increase or decrease is by at least 10%, preferably by at least 20%, more preferably by at least 50%, still more preferably by at least 75%, yet more preferably by at least 95% and most preferably by at least 100% relative to an untreated control organism, sample, or molecule.

“Immunomodulation” or “immunomodulatory” refers to an alteration in the overall immunoreactivity of the immune system in a mammal, or alteration in the response of a cell, relative to an untreated control of the same type, upon treatment with an agent, such as a polypeptide or nucleic acid molecule of the present invention, or fragments and analogs thereof. Immunomodulation can be assayed using immune cells, for example, B cells, T cells, antigen-presenting cells, or any other cell that is involved in immune function. Immunomodulation can also be assayed by determining expression and/or activity of immune-related genes and proteins, or immune-related compounds, such as cytokines, cytokine receptors, immunoglobulins, etc.

“Immunosuppression” refers to a decrease in the overall immunoreactivity of the immune system upon administration of an immunomodulator in comparison to the immunoreactivity of an immune system that has not been contacted with the particular immunomodulator. “Immunostimulation” refers to an increase in the overall immunoreactivity of the immune system upon administration of an immunomodulator in comparison to the immunoreactivity of an immune system that has not been contacted with the particular immunomodulator. “Decreasing T cell stimulation” means lowering the level of T cell stimulation as measured by, for example, a chromium release assay. “Decreasing inflammation” means decreasing the number of inflammatory cells (leukocytes, for example eosinophils) in the target tissue by, preferably, two-fold. By “cell proliferation” is meant the growth or reproduction of similar cells. By “apoptosis” is meant the process of cell death where a dying cell displays a set of well-characterized biochemical hallmarks which include cytolemmal blebbing, cell soma shrinkage, chromatin condensation, and DNA laddering.

“Immunomodulator” refers to an agent that induces an immunomodulatory effect or alteration (i.e., immunosuppression, immunostimulation, etc.) as measured, for example, by an alteration of virulence in mutated viruses or a variety of immunoassays well known in the art (for example, chemotaxis assays as described herein). For example, in the present invention, an immunomodulator may elicit an altered level of immune function, such that the alteration in the level of immune function identifies a viral 2L polypeptide. An “anti-inflammatory” agent is an immunomodulatory agent capable of decreasing the overall inflammation or immune function upon administration to an individual.

### Identification of 2L-TNFα Interface

From the crystal structure and analysis thereof, as described throughout and specifically detailed in Example I and Table 4, the amino acid residues participating in the 2L-TNFαt interface of the 2L-TNFα complex are identified as shown in Table 1.

TABLE 1
Table 1. Interactions between 2L and TNFα.

| 2L | | % | TNFα | | |
|---|---|---|---|---|---|
| 2L Residue | Location | ASA | Residue | (subunit) | Location |
| HIS58* | α1 helix | 4 | TYR87 | (B) | D-E loop |
| GLU99* | β5 strand | 6 | ARG31 | (A) | A-A′ loop |
| GLU99* | β5 strand | | ARG32 | (A) | A-A′ loop |
| GLY101 | β5-β6 loop | 2 | ARG31 | (A) | A-A′ loop |
| PHE104 | β6 strand | 6 | ASN30 | (A) | A-A′ loop |
| PHE104 | β6 strand | | ARG31 | (A) | A-A′ loop |
| LYS125 | β7-β8 loop | 5 | SER86 | (B) | D-E loop |
| GLY157 | α2 helix | 3 | SER86 | (B) | D-E loop |
| GLY157 | α2 helix | | TYR87 | (B) | D-E loop |
| TYR160 | α2 helix | 9 | ARG82 | (B) | D strand |
| TYR160 | α2 helix | | SER86 | (B) | D-E loop |
| TYR160 | α2 helix | | GLU127 | (B) | F-G loop |
| MET161 | α2 helix | 8 | ASN34 | (A) | A-A′ loop |
| MET161 | α2 helix | | ARG82 | (B) | D strand |
| MET161 | α2 helix | | VAL91 | (B) | E strand |
| GLY162 | α2 helix | 2 | TYR87 | (B) | D-E loop |
| LEU165 | α2 helix | 3 | ALA33 | (A) | A-A′ loop |
| LEU165 | α2 helix | | VAL91 | (B) | E strand |
| LEU168 | α2 helix | 3 | ARG32 | (A) | A-A′ loop |
| GLN171 | α2 helix | 4 | GLN21 | (A) | A-A′ loop |
| LYS175 | α2 helix | 4 | GLN21 | (A) | A-A′ loop |
| GLU225* | α3 insertion | 2 | ARG44 | (A) | B′ strand |
| VAL229 | α3 insertion | 5 | GLU42 | (A) | B′ strand |
| VAL229 | α3 insertion | | ARG44 | (A) | B′ strand |
| ASN230 | α3 insertion | 7 | ASN30 | (A) | A-A′ loop |
| ASN230 | α3 insertion | | LEU37 | (A) | A′ strand |
| ASN230 | α3 insertion | | LEU43 | (A) | B′ strand |
| GLY231 | α3 insertion | 6 | LEU37 | (A) | A′ strand |
| GLY231 | α3 insertion | | VAL41 | (A) | B′ strand |
| GLY231 | α3 insertion | | GLU42 | (A) | B′ strand |
| ARG232* | α3 insertion | 5 | GLU42 | (A) | B′ strand |
| ARG232 | α3 insertion | | ARG44 | (A) | B′ strand |
| GLU289 | F-G loop | 4 | GLN27 | (A) | A-A′ strand |
| GLU289* | F-G loop | | ARG31 | (A) | A-A′ strand |

% ASA (accessible surface area) = percent of the total interface ASA contributed by each residue.
Residues contributing less that 2% of interface ASA were excluded unless they are involved in hydrogen bonds or salt bridges (indicated by asterisks).

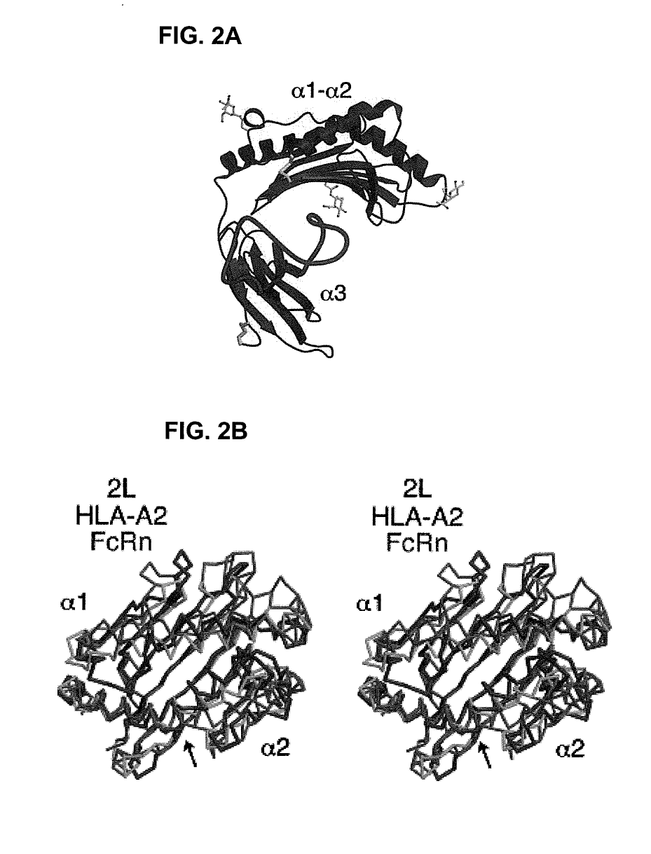

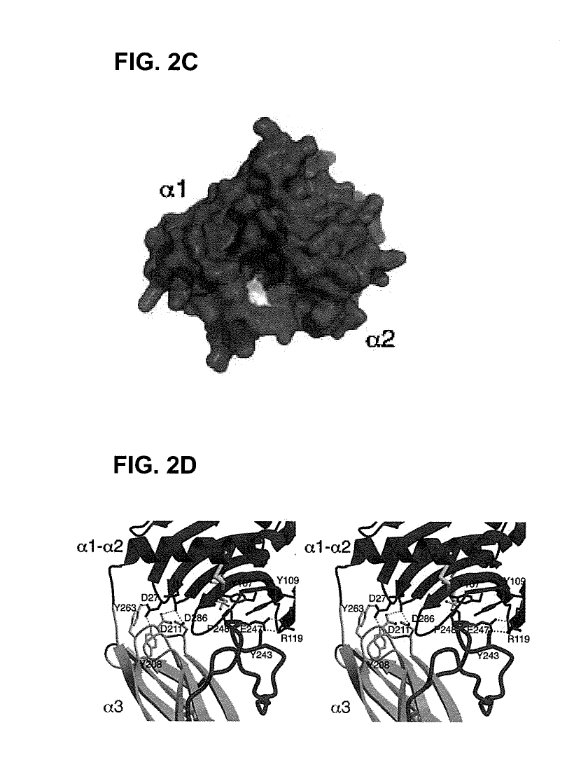

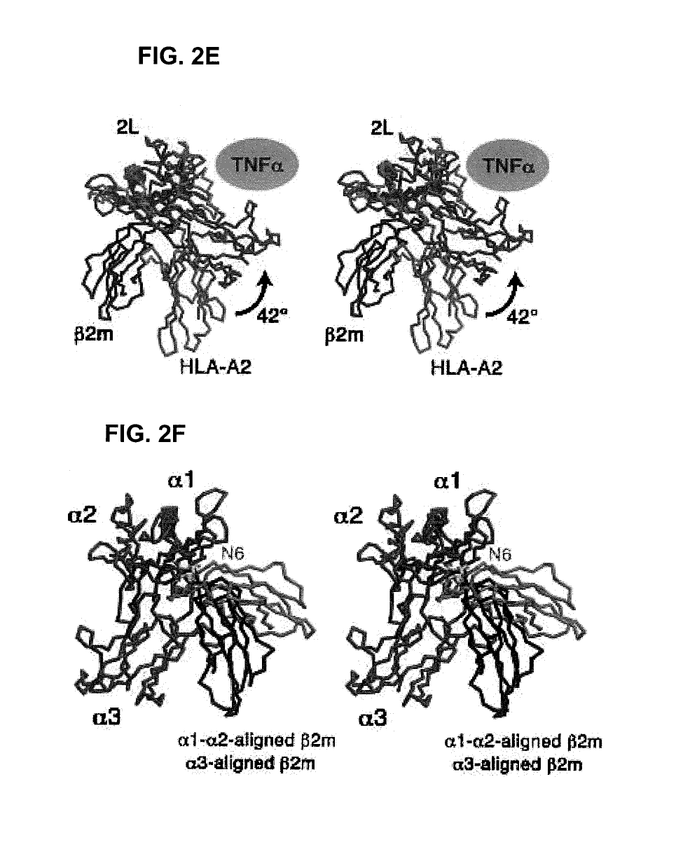

The analysis and modeling of the atomic coordinates (Table 4) of the 2L-TNFα crystal structure reveal that 2L binds to TNFα using a large and highly complementary interface as represented in FIGS. 2A, 2B, 2C). The high (picomolar) affinity between 2L and TNFα (Brunetti et al., 2003, PNAS, 100:4831-4836) is rationalized by the properties of the interaction. For example, the TNFα binding site on 2L spans a long (nearly 40 Å) distance, and the total buried surface area at the 2L-TNFα interface (2060 Å², of which 1030 Å² are contributed by 2L, and 710 Å² and 320 Å² are contributed by adjacent TNFα subunits in the trimer) is considerably larger than the areas of typical protein-protein recognition interfaces (1560-1700 Å²) (Jones et al., 1996, PNAS, 93:13-20).

The interface also exhibits a high degree of complementarity as assessed by the shape complementarity index, S_{C}, an index that varies from 0 (not complementary) to 1 (a perfect fit) (Lawrence, 1993, J Mol Biol. 234: 946-950). The S_{C} value for the 2L-TNFα interface is 0.72, indicating a more complementary interface than typical antibody-antigen interfaces with S_{C} values of 0.64-0.68 (Lawrence, 1993, J Mol. Biol. 234: 946-950). By comparison, the TNFR1/TNFβ interface has a lower shape complementarity index (S_{C}=0.64), but buries a similar surface area (2120 Å², with 1090 Å² from TNFR1 and 530 Å² and 510 Å² from adjacent TNFβ subunits).

Despite sharing only ˜13% sequence identity with human class I MHC heavy chains and lacking the β2m light chain, the 2L protein adopts a class I MHC-like fold including an α1-α2 superdomain, comprising an anti-parallel β-sheet topped by two α-helices, and an α3 domain with an immunoglobulin constant region β-sandwich fold (FIG. 2A). Disulfide bonds are found in the expected positions for class I MHC molecules and homologs: linking the α2 domain helix to the β-sheet floor of the α1-α2 platform, and joining the two β-sheets in the α3 domain. In addition, there is a disulfide bond near the C-terminus joining residues separated by only three residues.

In a comparison with structures of classical class I MHC molecules and other class I MHC homologs, including CD1, HFE, FcRn, ZAG, MIC-A, M10.5, T-22, RAE-1 and viral class I homologs (UL18, m144 and m157), the α1-α2 superdomain of 2L was most similar to that of a classical MHC protein, HLA-A2 with a root mean square deviation (RMSD) of 1.4 Å for 104 Cα atoms) (Table 2). However, in common with most class I homologs (HFE, FcRn, MIC-A, T-22, RAE-1, and the viral class I homologs m144 and m157), 2L does not contain a noticeable groove between its α1 and α2 domain helices (groove surface area of 60 Å²) and no unexplained electron density was observed in this region. In this regard, 2L is more similar to HFE and FcRn (groove surface areas of 415 Å² and 235 Å², respectively) (Lebron et al., 1998, Cell, 93:111-123), which do not present endogenous peptides, than it is to classical peptide-binding class I MHC molecules (groove surface areas of ˜760 Å²), the peptide-binding viral homolog UL18 (groove surface area of 900 Å²), or the lipid-presenting CD1d1 protein (groove surface area of 1440 Å²) (Yang, 2008, PNAS, 105: 10059-10100; Lebron et al., 1998, Cell, 93:111-123; Olson et al., 2005, PLoS Biol., 3: e257).

TABLE 2
Root mean square deviations (RMSD) for superpositions of the α1-α2
platform of 2L with platforms of MHC and MHC-like molecules.

| Protein | RMSD | Cαs aligned | PDB code |
|---|---|---|---|
| HLA-A2 | 1.43 Å | 104 atoms | 2CLR |
| M10.5 | 1.45 Å | 105 atoms | 1ZS8 |
| UL18 | 1.56 Å | 105 atoms | 3D2U |
| ZAG | 1.69 Å | 107 atoms | 1ZAG |
| T22 | 1.70 Å | 96 atoms | 1C16 |
| FcRn | 1.73 Å | 123 atoms | 1EXU |
| CD1d1 | 1.75 Å | 113 atoms | 1CD1 |
| HFE | 1.77 Å | 102 atoms | 1A6Z |
| m144 | 1.81 Å | 90 atoms | 1U58 |
| m157 | 1.83 Å | 80 atoms | 2NYK |
| MIC-A | 1.85 Å | 103 atoms | 1HYR |
| RAE-1 | 1.91 Å | 91 atoms | 1JFM |

### Data Analysis of Crystal Structure

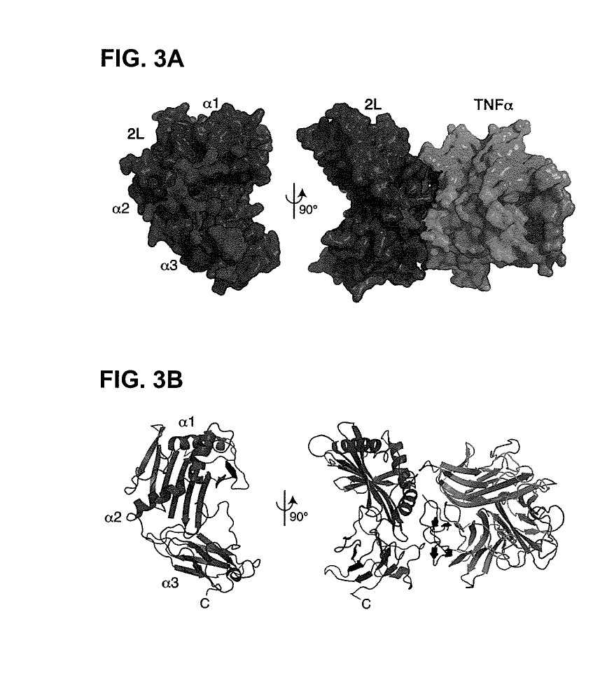

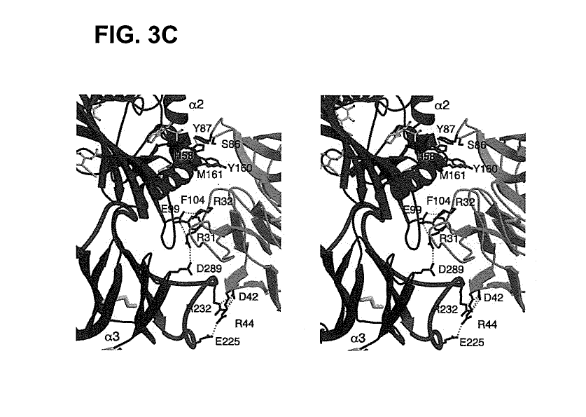

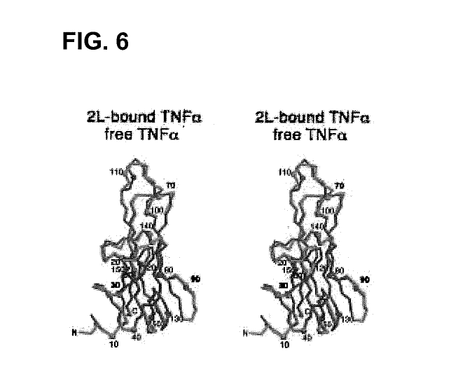

The structure of the 2L-bound TNFα trimer resembles its counterparts in structures of unliganded TNFα (Eck and Sprang, 1989, J Biol Chem, 264:17595-17605) and TNFR1-bound TNFβ (Banner et al., 1993, Cell, 73: 431-445) (FIG. 6) (RMSD of 0.91 Å for 148 Cα atoms for the TNFα-TNFα comparison, and 0.95 Å for 134 Cα atoms for the TNFβ-TNFα comparison), consisting of protomers with two antiparallel β-sheets with a jellyroll folding topology. The relatively modest conformational changes in the 2L-bound TNFα structure include movement of the A-A′ loop (residues 31-35) located at the 2L binding site. This different conformation permits two salt bridge interactions: 2L Glu99 with TNFα Arg31 and Arg32, and 2L G1u289 with TNFα Arg31 (FIG. 3C). The corresponding region of TNFβ, residues 48-52, also exhibits changes in mainchain and sidechain torsion angles upon binding TNFR1 (Banner et al., 1993). Smaller changes were also observed in the other binding site loops of TNFα, including residues 144-148 and 21-25 (FIG. 6). In one embodiment, a TNFa-binding candidate compound confers a conformational change in residues 31-35 (ARG31, ARG32, ALA33, ASN34, ALA35), residues 21-25 (GLN21, ALA22, GLU23, GLY24, GLN25), and residues 144-148 (PHE144, ALA145, GLU146, SER147, GLY148) of TNFα (SEQ ID NO: 14).

2L binds to TNFα using residues from all three of its domains to interact with the region surrounding the shallow groove between adjacent TNFα subunits, burying loops D-E and A-A′, which are also critical for TNFα binding to TNFR1 and TNFR2 (Mukai et al., 2009, J Mol. Biol. 385: 1221-1229) and TNFβ binding to TNFR1 (Banner et al., 1993, Cell, 73:431-445) (FIGS. 1, 3). A major component of the 2L-TNFα interface involves TNFα D-E loop residues 86-87 from one subunit and A-A′ loop residues 30-34 from the neighboring subunit interacting with 2L α2 domain helix residues 157-175. For example, the sidechain of TNFα residue Tyr87 in the D-E loop, a critical residue for binding both TNFRs (Mukai et al., 2009), contacts the kink in the 2L a2 domain helix and forms a hydrogen bond with His58 from the α1 domain helix. Some features of the TNF binding site for 2L and host TNFRs differ: G-H loop residues Asp143 and Ala145 and residues in the E-F loop, which are part of the TNFα binding site for TNFR1 (Loetscher et al., 1993, J Biol Chem, 268: 26350-26357) do not contact 2L, and TNFα strand B′, which interacts with the insertion in the 2L a3 domain, is not part of TNF's binding site with host receptors.

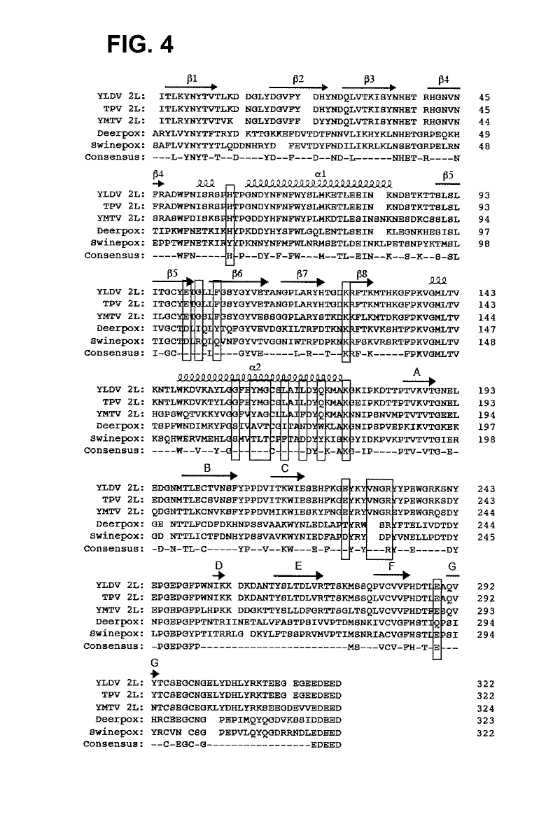

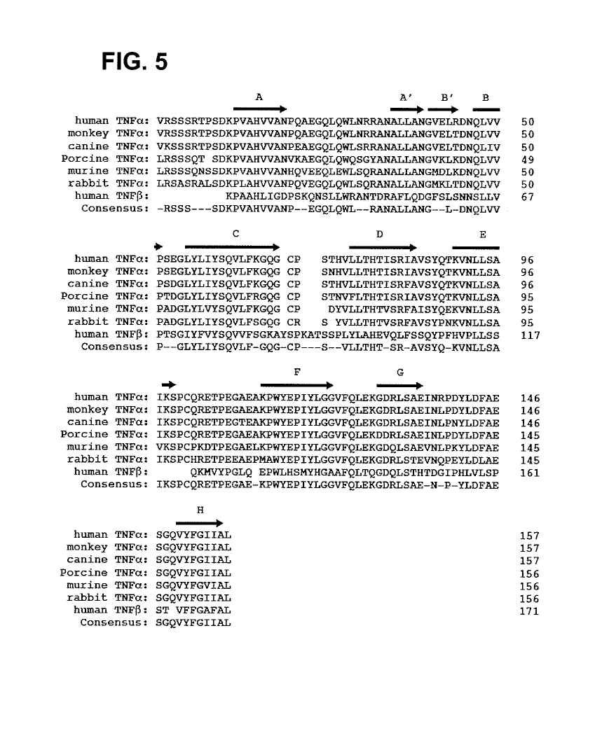

Consistent with the observation that poxvirus 2L proteins show varying degrees of specificity for binding TNFα from different species (Rahman et al., 2006, J Biol Chem, 281: 22517-22526, which is incorporated herein by reference), the TNFα binding site on 2L is less conserved than the rest of the protein: 26% (5 of 19) of the binding site residues are conserved in 2L proteins from four or more poxviruses, compared with an overall conservation of 42% (125 of 299 residues) (FIG. 4). Sequence diversity at the binding site is likely related to adaptation of poxviruses to the TNFα of their host species. Similarly, variability in classical class I MHC molecules primarily maps to a different location, the peptide-binding groove, resulting in distinct allele-specific peptide-binding preferences (Madden, 1995, Annu Rev Immunol, 13:587-622). Accordingly, in one embodiment, a TNFa-binding candidate compound is a 2L-derived polypeptide wherein the starting 2L protein is YLDV 2L (SEQ ID NO: 1) or TPV 2L (SEQ ID NO: 2), and the candidate compound has at least one amino acid modified that is not one of the 2L interface residues. Upon modification, the residue position of the interface amino acids may be altered without necessarily imparting a change to the structure of the 2L interface or its binding to the TNFα interface residues. Therefore, the positions of the amino acids at the interface need not necessarily correspond to the positions prior to modification, although it may be desirable for them to do so.

In another embodiment, a TNFa-binding candidate compound is a 2L-derived polypeptide wherein the starting 2L protein is YMTV 2L (SEQ ID NO: 3). YMTV 2L binds monkey TNFα with a binding affinity (K_{d}) of 230 picomoles, and binds human TNFα with a K_{d} of 440 picomoles (Rahman et al., 2006). The binding affinity of YMTV 2L derived TNFa-binding candidate compounds for human TNFα may therefore be reduced as compared with YLDV 2L or TPV 2L derived compounds, which can be advantageous in the treatment of certain immunomodulatory diseases.

Unlike naturally occurring host TNFRs, 2L does not bind to TNF or other TNF family members (Brunetti et al. 2003, PNAS, 100:4831-4836). To better understand this desirable specificity for binding TNFα, TNFβ was superimposed on the 2L-TNFα structure (FIG. 6). Although mainchain atom steric clashes were not apparent, the specificity of 2L for TNFα may be related to the complementarity of acidic residues (2L G1u99 and Asp289) to basic residues in the TNFα A′-A″ loop (Arg31 and Arg32) (FIG. 2C), which are not present in TNF (FIG. 5). In addition, binding of TNFβ may be prevented by different positions of the TNF D-E loops (residues 85-89 in TNFα; 106-110 in TNFβ), which differ by ˜2.5 Å, as measured between the Cα atoms of analogous residues (TNFα Tyr87 and TNFβ Tyr108). It would therefore be desirable to preserve these 2L residues and/or the relative positions thereof with reference to TNFa during any modification of the starting 2L sequence to form a candidate compound.

### Candidate TNFα-Binding Compound

In one aspect of the invention, a method for designing a therapeutic TNFα binding candidate is provided. In one embodiment, the method comprises first providing a 2L protein having an amino acid sequence of SEQ ID NOs: 1, 2, or 3. The method then comprises modifying the 2L protein without modification to any of the 2L interface residues, which are: HIS58, GLU99, GLY101, PHE104, LYS125, GLY157, TYR160, MET161 or ALA161, GLY162, LEU165, LEU168 or PHE168, GLN171, LYS175, GLU225, VAL229, ASN230, GLY231, ARG 232, and GLU289, wherein MET 161 and LEU168 correspond to SEQ ID NOs: 1-2 (YLDV and TPV 2L) and ALA161 and PHE168 correspond to SEQ ID NO: 3 (YMTV 2L).

In another embodiment of the method, similar, or equivalent amino acids may be substituted for those disclosed above at the 2L-TNFα interface. Conservative substitutions include substitutions within the following groups: glycine, alanine, valine, isoleucine, and leucine; aspartic acid, glutamic acid, asparagine, and glutamine; serine and threonine; lysine and arginine; and phenylalanine and tyrosine.

In the foregoing aspect of the invention, it is desirable that the candidate either binds to the TNFα interface residues participating in the 2L-TNFα interface or prevents binding of a TNFR to the TNFα interface residues participating in the 2L-TNFα interface. The TNFR may be TNFR1. The TNFα-binding candidate may include the amino acid residues shown in Table 1, having at least 2% accessible surface area (ASA) or contributing to hydrogen bonds or salt bridges.

In another aspect of the invention, a method for designing a TNFα-binding candidate is provided wherein the method comprises providing a candidate that has a shape complementarity index, (S_{c}) value that is from 0.64 to 0.78. It should be noted that the S_{c} for TNFR1/TNFβ is 0.64, and S_{c} for typical antibody-antigen interfaces is from 0.64 to 0.68 (Lawrence, 1993, J Mol. Biol. 234: 946-950). The candidate compound may have an S_{c} value that is from 0.64 to 0.72 or about 0.72. The candidate compound may have an S_{c} value that is from 0.65 to 0.78, from 0.66 to 0.78, from 0.67 to 0.78, from 0.68 to 0.78, from 0.69 to 0.78, from 0.70 to 0.78, from 0.71 to 0.78, from 0.72 to 0.78, from 0.73 to 0.78, from 0.74 to 0.78, from 0.75 to 0.78, from 0.76 to 0.78, or from 0.77 to 0.78.

In one aspect of the invention, a method for designing a candidate TNFα-binding compound is provided wherein the candidate binds to TNFα amino acid residues TYR87, ARG31, ARG32, ASN30, SER86, ARG82, GLU127, ASN34, VAL91, ALA33, VAL91, GLN21, ARG44, GLU42, LEU37, LEU43, VAL41, and GLN27.

In one embodiment, a method for designing a candidate TNFa-binding compound is provided wherein the candidate amino acid sequence is not a 2L protein, but includes the atomic coordinates of the 2L interface of Table 1 and Table 4 within an acceptable 1.0 Angstrom root mean square deviation (RMSD) (Baber et al., 2009, J. Chem. Inf. Model., 49 (8): 1889-1900).

In one embodiment, a method for designing a candidate TNFa-binding compound is provided wherein starting with a 2L protein sequence (e.g. SEQ ID NOs: 1, 2 or 3), the candidate amino acid sequence is provided to a webserver that predicts potential MHC class I epitopes from a peptide sequence (e.g. the SYFPEITHI web server). Of the amino acids predicted to be epitopes, those which participate in the 2L-TNFα interface, or which form part of the scaffold proximal and/or crucial to the interface, can be modified, but modification may not be desired given their critical role for 2L binding to TNFα. Accordingly, modification of the 2L polypeptide may at least initially be guided by modifying those amino acids that are predicted to fall within an MHC I epitope and are not in the 2L interface (Example 1). With respect to epitopes, mutation of one residue can eliminate the epitope (Griffith et al., 1987, J of Immunol., 138: 4480-4483; Smahel et al., 2008, Cancer Immunol, Immunotherapy, 57: 823-831). Similarly, in another embodiment, a 2L peptide sequence is provided to a webserver that predicts MHC II and/or B-cell epitopes.

### Expression/Purification of 2L-Derived TNFα Binding Candidates

TNFα binding candidate DNA constructs are made as disclosed (Rahman et al., 2009 Virology 386:462-468, which is incorporated herein by reference). A DNA plasmid, for example, plasmid pcDNA-2L, contains the complete YLDV 2L (SEQ ID NO: 4), TPV 2L (SEQ ID NO: 5) or YMTV 2L (SEQ ID NO: 6) open reading frame. The desired mutated sequences are PCR amplified with specific oligonucleotide primers using a DNA polymerase (e.g. Pfu DNA polymerase, Stratagene, La Jolla, Calif.), and the pcDNA 2L as template.

The TNFα candidate plasmids are transferred into an expression vector for transfection and expression in an expression system. For example, the pcDNA plasmid is transferred into pFastBacI plasmid, (Invitrogen, Carlsbad, Calif.) and recombinant baculoviruses are produced using the Bac-to-Bac expression system following the manufacturer protocols. In brief, the plasmids are transformed into competent DH10Bac bacteria where a transposition event generated the corresponding recombinant bacmids. The purified bacmid DNA are transfected into Sf21 insect cells and the recombinant baculoviruses are harvested from the cell culture supernatant 3-5 days after transfection.

Screening for Compounds that Bind to TNFα Interface Residues

The 2L-TNFα crystal structure as disclosed herein is useful in methods of creating or identifying candidate compounds using a structure-based approach. As is known in the art, screens to identify agonist and antagonist molecules for TNFα can be used. Accordingly, using the methods as disclosed, for example, in Kolb et al., 2009, PNAS, 106: 6843-6848 and Casey et al., 2009, J Chem Inf Model, 49: 2708-2717, a test compound that binds to TNFα at the TNFα interface residues or prevents binding of h′TNFR1 and/or hTNFR2 to the TNFα interface residues can be identified as a therapeutic candidate compound. The test compound can optionally confer a desired conformational change an/or have a desired binding affinity. The test compound may be a small molecule, polypeptide, protein, aptamer, antibody, fusion protein or nucleic acid comprised of naturally occurring or artificial constituents. In one embodiment, a test compound is screened for its ability to bind the TNFα interface residues TYR87, ARG31, ARG32, ASN30, SER86, ARG82, GLU127, ASN34, VAL91, ALA33, VAL91, GLN21, ARG44, GLU42, LEU37, LEU43, VAL41, and GLN27.

### Modification of TNFα Candidate Compound

In one embodiment, a modification is made to a 2L-derived TNFa-binding candidate comprising modifying one or more of any residue of a 2L protein (SEQ ID NOs: 1-3) that is not in the 2L interface. The modification may cause the candidate to have increased or decreased TNFα binding activity compared to the original 2L protein, wherein the reduction or increase in the activity is a direct result of the modification to the amino acid residues not in the 2L interface.

In one embodiment, a candidate compound designed in accordance with the present invention may be made by any means known in the art. For example, PCR can be used to generate a polynucleotide comprising codons that result in a mutation in one or more of the corresponding non-interface 2L amino acid residues. Commercial kits are also available for producing mutants, such as QuikChange™ (Stratagene, La Jolla, Calif.). In addition, site directed mutagenesis may be employed, as well as chemical synthesis. The skilled artisan is well versed in the methods available for producing the polynucleotides and polypeptides of the present invention.

### Affinity Binding of Candidate Compounds

The affinity of the candidate compound to TNFα is measured using one of several methods known in the art. For example, (Rahman et al., 2006, J Biol Chem 281:22517-22526) recombinant candidate proteins are immobilized at low densities of ˜500 RU on CM5 chips and varying concentrations of TNF alpha protein is applied to the chips. Following an association period of 120 seconds, running buffer HBS-EP is injected to monitor the dissociation phase of binding. In one embodiment, the candidate binding compound has an affinity to TNFα of from about 43 picomoles (pm) to 440 pm (Rahman et al. 2006, J Biol Chem, 281: 22517-22526). In one embodiment, the candidate binding compound of the present invention has a binding affinity to TNFα of from about 40 pm to about 445 pm. Alternatively, the candidate TNFα-binding compound has a IQ value that is from 40 pm to 440 pm, from 40 pm to 430 pm, from 40 pm to 420 pm, from 40 pm to 410 pm, from 40 pm to 400 pm, from 40 pm to 390 pm, from 40 pm to 380 pm, from 40 pm to 370 pm, from 40 pm to 360 pm, from 40 pm to 350 pm, from 40 pm to 340 pm, from 40 pm to 330 pm, from 40 pm to 320 pm, from 40 pm to 310 pm, from 40 pm to 300 pm, from 40 pm to 290 pm, from 40 pm to 280 pm, from 40 pm to 270 pm, from 40 pm to 260 pm, from 40 pm to 250 pm, from 40 pm to 240 pm, from 40 pm to 230 pm, from 40 pm to 220 pm, from 40 pm to 210 pm, from 40 pm to 200 pm, from 40 pm to 190 pm, from 40 pm to 180 pm, from 40 pm to 170 pm, from 40 pm to 160 pm, from 40 pm to 150 pm, from 40 pm to 140 pm, from 40 pm to 130 pm, from 40 pm to 120 pm, from 40 pm to 110 pm, from 40 pm to 100 pm, from 40 pm to 90 pm, from 40 pm to 80 pm, from 40 pm to 70 pm, from 40 pm to 60 pm, or from 40 pm to 50 pm.

In an alternative embodiment, the candidate binding compound of the present invention decreases the binding affinity to TNFα. For example, a method for decreasing affinity to TNFα compared to any of the full length 2L proteins may be desired in order to attenuate binding. As such, in one embodiment, a method for a 2L-derived candidate compound is provided comprising modifying one or more amino acid residues of a 2L protein within the 2L interface residues. In another embodiment, a 2L-derived candidate compound that has decreased binding to TNFα compared to 2L, has a modification in one more amino acid residues of the 2L interface in addition to one or more amino acid residues that do not participate in the 2L interface.

### Competitive Binding

To determine the binding preference for a candidate TNFα binding compound with respect to known TNFα binding proteins, e.g. 2L, TNFR2, etc, a competitive binding assay is used. Competitive/inhibition binding assays are known in the art. A preferred ELISA method for determining candidate TNFα binding compounds and their affinity by competitive inhibition is disclosed in Brunetti et al. 2003, PNAS, 100:4831-4836. In one embodiment, the candidate TNFα-binding compound of the present invention competitively inhibits the binding of TNFα to TNFR2. In one embodiment, the competitive binding of the candidate compound inhibits the binding of TNFα to TNFR1 at least 30%, preferably at least 50%, more preferably at least 75%, and most preferably at least 95% compared to 2L polypeptide.

### Therapeutic Methods Employing TNFα Candidate Binding Compound

The invention includes methods of treating or preventing immunological disorders by using immunomodulatory agents. 2L-derived TNFα binding polypeptides identified in the present invention that exhibit anti-cytokine activity, anti-inflammatory activity, and/or exhibit a decrease in leukocyte chemotaxis activity are considered particularly useful in the invention; such polypeptides may be used, for example, as therapeutics to decrease the immunoreactivity in an individual with rheumatoid arthritis. Other immunological disorders that may be treated using an immunosuppressive agent, or an agent that reduces the immune function, include acute inflammation, allergic reactions, asthmatic reactions, inflammatory bowel diseases (i.e., Crohn's Disease and ulcerative colitis), transplant rejection, and restenosis. Alternatively, a polypeptide that enhances or induces apoptosis may be used in the treatment of tumors.

Treatment or prevention of diseases resulting from an immunomodulatory disorder is accomplished, for example, by modulating the function of an immunoregulatory protein by delivering a 2L-derived TNFα candidate protein to the appropriate cells. It is also possible to correct an immune defect by modifying the physiological pathway (e.g., a signal transduction pathway), in which the immunoregulatory protein participates, by delivering a TNFα candidate protein or nucleic acid molecule to the appropriate cells.

Direct administration of a recombinant TNFα protein, nucleic acid molecule, antibody, or compound, either to the site of a potential or actual disease-affected tissue (for example, by injection), or systemically for treatment of, for example, an autoimmune or inflammatory disorder, can be performed accordingly to any conventional recombinant protein administration technique known in the art or described herein. The actual dosage depends on a number of factors known to those of ordinary skill in the art, including the size and health of the individual patient, but generally, between 0.1 mg and 100 mg inclusive are administered per day to an adult in any pharmaceutically-acceptable formulation.

A TNFα immunomodulator, antiflammatory agent, or anticarcinogen may be administered with a pharmaceutically-acceptable diluent, carrier, or excipient, at a pharmaceutically effective dose. Conventional pharmaceutical practice may be employed to provide suitable formulations or compositions to administer TNFα candidate compounds to patients suffering from an immunomodulatory disorder. Any appropriate route of administration may be employed, for example, parenteral, intravenous, subcutaneous, intramuscular, intracranial, intraorbital, ophthalmic, intraventricular, intracapsular, intraspinal, intracisternal, intraperitoneal, intranasal, aerosol, or oral administration. Therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found in, for example, “Remington's Pharmaceutical Sciences, supra.” Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for TNFα candidate immunomodulatory compounds include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

### Animal Models

2L-derived TNFα binding immunomodulators of this invention and other compounds found to be effective at the level of protein expression or activity are tested in animal models, for efficacy in treating, for example, autoimmune and inflammatory diseases and cancer. In one embodiment, a method of testing a candidate TNFα-binding compound is provided, comprising: selecting a candidate compound that binds to TNFα; assaying the compound for a disease in an animal model for the disease; and, determining if the compound decreases the disease in the animal model in comparison to known compounds (i.e. control inhibitors)—e.g. TNFα antibodies, as described (U.S. Pat. No. 7,070,775) and etanercept, (Enbrel™) (U.S. Pat. No. 5,712,155).

Animal models for testing the immunomodulatory effects of candidate compounds are well known in the art. Therefore, the present invention refers to a selection of animal models that can be used to test the candidate compounds of the invention. Animal models proposed for use in the present invention to test candidate compounds for their efficacy in treating autoimmune and inflammatory disorders include, but are not limited to:

### Acute Inflammation:

Animal models of acute inflammation are targeted for initial and rapid drug efficacy screening, and for their potential predicative value of outcomes in chronic inflammatory diseases. The following animal models can be used to test the candidate compounds of the present invention for their efficacy in treating acute inflammation: 1) carrageenin-induced inflammation model; 2) turpentine-induced inflammation model; 3) transgenic HLAB-27 inflammation model; and 4) ear-scratch model of inflammation.

### Rheumatoid Arthritis: Rat, Mouse, Rabbit

Efficacy in Rheumatoid Arthritis evaluated in: 1) various antigen-induced arthritis models in rabbit, rat, and mouse; and 2) in transgenic rheumatological models.

The molecular and cellular mechanisms of action of the candidate compounds are evaluated by testing their efficacy in influencing key intracellular mechanisms that regulate degradative processes involved in joint disease. Important molecular and cellular mechanisms that receive particular attention include signaling events regulating disease processes such as increased angiogenesis, synovial hyperplasia, and matrix metalloprotease expression. These processes have been implicated in cartilage degradation in arthritic diseases.

1. Collagen-induced Arthritis: Rat, Mouse, Rabbit

Autoimmune-mediated polyarthritis can be induced in certain strains of rodents (rat, mouse and rabbit) and non-human primates by immunizing them with native type II collagen. The collagen-induced arthritis model is widely used and well characterized. Collagen-induced arthritis is mediated by susceptibility to autoantibodies which bind to a particular region of type II collagen. The mechanism of induction is linked to MHC-class II molecules but also depends on the species of type II collagen used for immunization.

2. Ovalbumin-induced Arthritis; Rabbit

Candidate compounds are tested for efficacy in decreasing signs and symptoms of ovalbumin arthritis. Polyarthritis is induced in rabbits by immunizing them with Ovalbumin.

3. Adjuvant-induced Arthritis: Rat, Mouse, Rabbit

Candidate compounds are tested for the efficacy in decreasing signs and symptoms of adjuvant-induced arthritis. Polyarthritis is induced in certain strains of rodents by immunizing them with Freud's Adjuvant.

4. Streptococcal Cell Wall-induced Arthritis: Rat

Candidate compounds are tested for efficacy in decreasing signs and symptoms of streptococcal cell wall-induced arthritis. Chronic, erosive polyarthritis is induced by intraperitoneal injection of an aqueous suspension of cell wall fragments isolated from group A streptococci.

Transplant Rejection (Acute and Chronic)

Efficacy in transplant rejection is evaluated in various models of graft vascular disease (GVD). GVD is the most common cause of late graft failure in solid organ transplantation. GVD or graft atherosclerosis is characterized by plaque formation and fibrosis in small vessels. The development of graft vascular disease has been associated with acute allograft rejection, ischemia-reperfusion injury, and bacterial or viral infections. The common pathway of these postoperative insults results in perivascular inflammation which triggers migration of mesenchymal cells into the vessel wall, eventually resulting in occlusion or partial occlusion of the vessel lumen.

1. Aortic Allograft Model: Rat, Rabbit, Monkey

Candidate compounds are tested for efficacy in reducing graft atherosclerosis and transplant rejection in a model of vascular injury after transplantation of aortic segments performed in certain strains of MHC mismatched rats, monkeys, and rabbits.

2. Tracheal Allograft Model: Rat, Rabbit, Monkey

Candidate compounds are tested for efficacy in reducing graft atherosclerosis and transplant rejection in a model of vascular injury after transplantation of tracheal segments performed in certain strains of MHC mismatched rats, rabbits, and monkeys.

3. Heterotopic Heart Transplant: Mouse, Rat, Monkey

A heterotropic heart transplantation is performed in MHC mismatched animals. In this model, animals are treated with cyclosporine A for only the first 7 days after transplantation are allowed to develop graft vascular disease, and are then analyzed after sacrifice at postoperative day 90.

4. Orthotopic Kidney Transplant: Mouse, Rat, Monkey

An orthotopic kidney transplantation is performed in MHC mismatched animals. In this model, animals receiving subtherapeutic doses of cyclosporine A for the first 10 days after transplantation are allowed to exhibit features of chronic renal allograft rejection in 70% of cases, and are then analyzed after sacrifice at postoperative day 90.

5. Orthotopic Lung Transplant: Rat, Monkey

Candidate compounds are tested for effectiveness in delaying or reducing signs and symptoms of organ rejection after lung whole organ transplantation in rats and monkeys.

6. Reperfusion Injury: Rat

The immediate postoperative course in clinical lung transplantation is often severely impaired by delayed graft function as a result of ischemia and reperfusion injury. Preventive efficacy of drug candidates in ischemia-reperfusion injury is evaluated using a model of acute in vivo double lung transplantation in the rat (Hausen et al., Ann. Thorac. Surg. 61:1714-9, 1996 incorporated herein by reference).

Restenosis

Candidate compounds are tested for efficacy in reducing atherosclerotic plaque deposition in a model of coronary restenosis after balloon angioplasty. Atherosclerotic plaque formation is critically involved in vascular occlusion and has been linked to excessive inflammatory and thrombotic response to arterial injury.

Asthma: Rodent

The effectiveness of candidate compounds in reduction of signs and symptoms of asthma is evaluated in rodent models of antigen-induced experimental airways inflammation. The models include:

1. Ovalbumin-Induced Experimental Airways Inflammation: Rodent

Candidate compounds are tested for efficacy in reducing inflammatory cell components in the bronchoalveolar lavage of the lungs after aerosol challenge in ovalbumin-sensitized rodent models of experimental airways inflammation.

2. Ovalbumin-induced Allergic Sensitization in Presence of GM-CSF Transgene Expression: Mice

Candidate compounds are tested for efficacy in reducing inflammatory cell components in the bronchoalveolar lavage of the lungs of mice after ovalbumin aerosol challenge in the context of local expression of GM-CSF (Staempfli et al., J. Clin. Invest., Vol. 102:9, 1704-1714).

Inflammatory Bowel Disease (IBD): Mice and Rats

Drug candidates are evaluated for their potential therapeutic efficacy in ulcerative colitis or Crohn's disease utilizing various models of antigen-induced and genetically-mediated spontaneous chronic intestinal inflammation in mice and rats. Examples include:

1. Dextran Sulfate Sodium Induced IBD: Mice

Chronic, irreversible clinical symptoms of IBD are induced by treating mice with an oral administration of dextran sulfate sodium.

2. Gene Deletion and Transgenic Models for IBD: Rodent

Compound efficacy will be tested in transgenic rodent lines which develop symptoms closely resembling the human elements of inflammatory bowel disease. Models include, the targeted deletion of the genes encoding IL-2, IL-10, TGF beta, T-cell receptor alpha/beta, keratin 8, Gi2 alpha. In addition, animals expressing transgenes for the human WA-B27 and HLA-B27 as well as a dominant negative construct which functionally blocks N-cadherin will be tested.

Uveitis

Drug candidates will be evaluated for efficacy in various animal models of uveitis. Key models include both, experimental autoimmune uveitis and adoptively transferred experimental autoimmune uveitis.

1. Experimental Autoimmune Uveitis (EAU)

EAU is a T-cell mediated inflammatory eye disease that can be induced in several mammalian species by immunization with ocular-specific antigens (Gery et al., Invest. Opthalmol. Vis. Sci., 27: 1296-1300, 1986. Sanui et al., J. Exp. Med., 169:1947-1989, incorporated herein by reference). This experimental disease is considered a model for a family of inflammatory eye diseases in humans and has been used to examine numerous modalities before their human testing.

2. Adoptively Transferred Experimental Autoimmune Uveitis

Adoptively transferred EAU is induced through injection of lymphocytes presensitized against the retinal antigen are injected into naive syngenic recipients (McAllister et al., J. Immunol., 138:1416-1420, 1987 incorporated herein by reference).

### EXAMPLES

### 1. Modified 2L Polypeptide

YLDV 2L amino acid sequence (SEQ ID NO: 1) was provided to the SYFPEITHI webserver. YLDV 2L residues 218-226 (ESEHFKGEY) are predicted to bind to MHC class 1 molecule HLA-A*01. The amino acids in positions 3 and 9 of this peptide fragment, E220 and Y226, respectively, are important to binding to HLA-A*01 (Falk et al., 1994, Immunogenetics, 40: 238-241). Examining the 2L-TNF crystal structure (Table 1 and Table 4), one can determine that changing TYR226 may be problematic since it appears to form part of a hydrophobic core stabilizing the 2L protein. On the other hand, GLU220 does not appear to have any structural role and does not contact TNFα. Thus one could predict that the mutant 2L protein E220S wherein a serine (SER) is substituted for the glutamic acid (GLU), would be stable, bind strongly to TNF-alpha, and have one less potential epitope for HLA-A*01.

### 2. Crystal Structure of 2L-TNFα Complex

Materials and Methods for purification of complexed 2L-TNFα protein crystal.

The gene for 2L was isolated by PCR from the Yaba-like disease virus (ATCC number VR-937) using a forward primer 2LF (SEQ ID NO: 7):

CATGGATAAGTTACTATTATTTAGCAC and reverse primer 2LR (SEQ ID NO: 8): TCAGTCTTCTTCATCCTCTTCACCTTCC. A C-terminally His-tagged version of 2L lacking six C-terminal residues (residues 1-316 of the mature protein) (SEQ ID NO: 18) was amplified by PCR using a forward primer JY152 (forward primer, with kozaq sequence and BamHI site) (SEQ ID NO: 9): CGCGGATCCCCTATAAATATGGATAAGTTACTATTATTTAGC and the reverse primer, JY154 (reverse primer stops at aa 332, with XhoI site) (SEQ ID NO: 10) CCGCTCGAGACCTTCTCCTTCTTCTGTTTTTCTATATAG. This C-terminally His-tagged 2L protein was subcloned into a dicistronic baculovirus transfer vector, pAcUW31 (PharMingen) subcloned with the human β2m gene. His-tagged 2L and β2m (both included native hydrophobic leader sequences) were expressed together in baculovirus-infected insect cells (Hi5 cells). In the numbering system used herein for 2L, the first amino acid of the mature 2L chain is residue 1; a previous numbering system, which started at the initial methionine of the signal peptide, refers to this residue as 17 (Brunetti et al., 2003, PNAS, 100: 4831-4836; Rahman et al., 2006, J Biol Chem, 281:22517-22526; Rahman et al., 2009, Virology, 386: 462-468). 2L was purified by nickel-NTA and gel filtration chromatography as a monomer lacking detectable β2m. An N-terminally His-tagged form of human TNFα (residues 6-157, numbered as in PDB entry 1TNF) (SEQ ID NO: 11) was amplified from an IMAGE TNFα cDNA clone using the TNF forward primer, (SEQ ID NO: 12) GACCATATGGGCAGCCATCATCATCATCATCACCGAACCCCGAGTGACAAGCCT G and TNR reverse primer, (SEQ ID NO: 13) GACCTCGAGTCACAGGGCAATGATCCCAAAG was expressed in the E. Coli cytoplasm using the expression vector pET23a (Novagen, EMD, Gibbstown, N.J.), and soluble TNFα (SEQ ID NO: 14) was purified from bacterial lysates by nickel-NTA and gel filtration chromatography.

Purified 2L and TNFα trimer were mixed at a 3:1 molar ratio and passed over a S200 16/60 gel filtration column in 20 mM Tris pH 7.5, 150 mM NaCl. The 2L-TNFα complex was isolated and then concentrated to 14 mg/ml and used to set up crystallization trials by hanging drop vapor diffusion. Crystals were obtained in 7-12% PEG 6K, sodium citrate pH 5.0-5.5, 0.2 M NaBr in monoclinic space group P2₁ (a=101.7, b=170.3, c=122.0, β=92.04°) with two 3:1 2L:TNFα complexes per asymmetric unit. The crystal used for data collections had been soaked in 0.5 mM methylmercury chloride, but no heavy atoms were found in the refined structure.

Structure Analysis. Data were processed using HKL2000 (Otwinowski et al., 1997, Meth Enzymol, 276: 307). The structure was determined by molecular replacement using a TNFα (Eck and Sprang, 1989, J Biol Chem, 264: 17595-17605) search model followed by location of 2L molecules using the coordinates of the α1-α2 platform of ZAG (PDB code 1T7V) (Delker et al., 2004, J Struct Biol, 148: 205-213) in a real space search (Sanchez et al., 1999, Science, 283: 1914-1919) (Table 3). After rigid-body refinement McCoy et al., 2007, J Appl Cryst, 40: 658), rebuilding was done using the program O (Jones et al., 1997, Methods Enzymol, 277: 173) and 2Fₒ-F_{c} annealed omit maps, alternating with refinement using CNS (Brunger et al., 1998, Acta Crystallogr D Biol Crystallogr, 54: 905-921). Non-crystallographic symmetry (NCS) restraints (300 kcal/mol) and group B factor refinement were used during refinement. The final model consisted of six NCS-related sets of atoms: 2L residues 1-299, TNFα residues 6-157, 16 water molecules, and 42 atoms of carbohydrate (ordered carbohydrate attached to 2L residues Asn6, Asn52, and Asn83, but not to Asn197, the final potential N-linked glycosylation site in the 2L sequence). The following cysteine pairs were disulfide bonded: 2L 97-163 (difference Fourier maps indicated some radiation-induced breakage of this bond (Burmeister, 2000, Acta Crystallogr D Biol Crystallogr, 56: 328-341), 202-281, 295-299, and TNFα 69-101. 2L residues 300-316, its C-terminal His-tag, and the N-terminal His-tag of TNFα were disordered.

For analysis of contacts and buried surface areas, the 2L α1-α2 platform was defined as residues 1-179 and the α3 domain was defined as residues 180-299. The CCP4 program AREAIMOL (CCP4, 1994, Acta Crystallogr D Biol Crystallogr, D50: 760-763) was used to calculate buried surface areas using a 1.4 Å probe. To identify interacting residues, a maximum distance of 4.0 Å for van der Waals interactions was used. Shape complementarity indices (S_{C}) were calculated as described (Lawrence, 1993, J Mol. Biol. 234: 946-950) using the S_{C} program in CCP4 (CCP4, 1994, Acta Crystallogr D Biol Crystallogr, D50: 760-763). Groove surface area calculations were performed as previously described (Lebron et al., 1998, Cell, 93: 111-123; Zeng et al., 1997, Science, 277: 339-345). Structural superpositions were performed using LSQMAN (Kleywegt, 1996, Acta Cryst D Biol Crystallogr, 52: 842-857). Figures were prepared with MOLSCRIPT (Kraulis, 1991, J Appl Crystallogr, 24: 946), RASTER-3D (Merritt et al., 1994, Acta Crystallogr D Biol Crystallogr, 50:869-873), and PyMOL (DeLano, 2002, The PyMOL Molecular Graphics System, Delano Scientific, San Carlos, Ca). The domain angle displacement for the 2L a3 domain relative to the HLA-A2 α3 domain (˜42° was calculated by measuring the angle between the long axes of each domain after alignment of the α1-α2 platforms of both molecules. The long axes of the α3 domains were defined using the coordinates of 2L residues G1y176 and Asn191 and HLA-A2 residues Glu177 and His192. The second rotation angle (˜40° relating the α3 domains was estimated using the angle defined by 2L Arg270 and Asn191 and HLA-A2 Va1248.

TABLE 3
Data collection and refinement statistics

| Space group | P2₁ |
|---|---|
| Unit cell (Å, degrees) | 101.7, 170.3, 122.0, |
| | 90, 92.04, 90 |
| Data collection | |
| Resolution (Å) | 50.0-2.80 (2.90-2.80) |
| Number of observations | 368612 |
| Unique reflections | 99161 |
| % Complete* | 96.8 (81.0) |
| I/σI | 16.5 (2.3) |
| R_{merge}(%)^{‡} | 8.1 (47.2) |
| Refinement | |
| Resolution (Å) | 50.0-2.80 |
| Reflections in working set \|F\| > 0 | 93732 |
| Reflections in test set \|F\| > 0 | 4965 |
| R_{cryst} (%)^{¶} | 23.6 |
| R_{free} (%) | 26.6 |
| No. of protein atoms | 21654 |
| No. of water molecules | 96 |
| No. of other atoms (carbohydrate) | 252 |
| Model geometry | |
| rmsd bond length (Å) | 0.007 |
| rmsd angles (deg) | 1.48 |
| Ramachandran plot | |
| most favored region (%) | 80.4 |
| additional allowed region (%) | 14.5 |
| generously allowed region (%) | 4.0 |
| disallowed region (%) | 1.0 |

Table 3. Values in parentheses indicate the high-resolution shells.
*Complete is the number of independent reflections/total theoretical number.
^{‡}R_{merge} (I) = (Σ|I(i) − <I(h)>|/ΣI(i)), where I(i) is the ith observation of the intensity of the hkl reflection and <I> is the mean intensity from multiple measurements of the h, k, l reflection.
^{¶}R_{cryst} (F) = Σₕ||F_{obs}(h)| − |F_{calc}(h)||/Σₕ||F_{obs}(h)|, where |F_{obs}(h)| and |F_{calc}(h)| are the observed and calculated structure factor amplitudes for the h, k, l reflection.
R_{free} is calculated over reflections in a test set not included in atomic refinement.

In summary, methods are disclosed for identifying a candidate TNFa-binding compound utilizing the structure, specifications and amino acid residues elucidated from the 2L-TNFα crystal structure. Methods are disclosed for assaying candidate TNFa-binding compounds for decreasing TNFα-mediated disease.

While the present invention has been illustrated and described with reference to certain exemplary embodiments, those of ordinary skill in the art will understand that various modifications and changes may be made to the described embodiments without departing from the spirit and scope of the present invention, as defined in the following claims.

TABLE 4
Coordinates for the YLDV 2L-TNFα complex crystal structure

| | |
|---|---|
| REMARK | coordinates from minimization refinement |
| REMARK | refinement resolution: 50.0-2.8 A |
| REMARK | starting r= 0.2382 free_r= 0.2663 |
| REMARK | final r= 0.2362 free_r= 0.2658 |
| REMARK | rmsd bonds= 0.007428 rmsd angles= 1.48430 |
| REMARK | wa= 4.25322 |
| REMARK | target= mlf cycles= 1 steps= 200 |
| REMARK | sg= P2(1) a= 101.656 b= 170.295 c= 122.002 alpha= 90.0 beta= |
| | 92.037 gamma= 90.0 |
| REMARK | parameter file 1: CNS_TOPPAR: protein_rep.param |
| REMARK | parameter file 2: CNS_TOPPAR: carbohydrate.param |
| REMARK | parameter file 3: CNS_TOPPAR: water.param |
| REMARK | molecular structure file: generate.mtf |
| REMARK | input coordinates: bgroup.pdb |
| REMARK | reflection file= p21reproc2.cv |
| REMARK | ncs= restrain ncs file = ncsr2.def |
| REMARK | Anisotropic B-factor tensor Ucart of atomic model without isotropic |
| | component: |
| REMARK | B11= 28.814 B22= −12.319 B33= −16.495 |
| REMARK | B12= 0.000 B13= −8.113 B23= 0.000 |
| REMARK | Isotropic component added to coordinate array B: 0.835 |
| REMARK | bulk solvent: probe radius=1, shrink value=1 |
| REMARK | bulk solvent: density level= 0.35 e/A\hat{ }3, B-factor= 46.1722 A\hat{ }2 |
| REMARK | reflections with \|Fobs\|/sigma_F < 0.0 rejected |
| REMARK | reflections with \|Fobs\| > 10000 * rms(Fobs) rejected |
| REMARK | fft gridding factor = 0.3333, B factor offset = 19.0633 A\hat{ }2, Elimit = 8 |
| REMARK | theoretical total number of refl. in resol. range: 101928 (100.0%) |
| REMARK | number of unobserved reflections (no entry or \|F\|=0): 3231 (3.2%) |
| REMARK | number of reflections rejected: 0 (0.0%) |
| REMARK | total number of reflections used: 98697 (96.8%) |
| REMARK | number of reflections in working set: 93732 (92.0%) |
| REMARK | number of reflections in test set: 4965 (4.9%) |
| CRYST1 | 101.656 170.295 122.002 90.00 92.04 90.00 P 21 |
| REMARK | FILENAME=“/Users/pamela/Desktop/apw/2L/p21/cns38new/xpand3/minimize.pd” |
| REMARK | DATE: 27-Mar-2009 16:51:11 created by user: pamela |
| REMARK | VERSION: 1.2 |

| | | | | | | | | | | | |
|---|---|---|---|---|---|---|---|---|---|---|---|
| ATOM | 1 | CB | ARG | A | 1 | −25.527 | −6.064 | 32.919 | 1.00 | 164.47 | A |
| ATOM | 2 | CG | ARG | A | 1 | −24.524 | −6.994 | 32.215 | 1.00 | 164.47 | A |
| ATOM | 3 | CD | ARG | A | 1 | −25.162 | −8.235 | 31.583 | 1.00 | 164.47 | A |
| ATOM | 4 | NE | ARG | A | 1 | −25.525 | −8.043 | 30.176 | 1.00 | 164.47 | A |
| ATOM | 5 | CZ | ARG | A | 1 | −24.654 | −7.821 | 29.193 | 1.00 | 164.47 | A |
| ATOM | 6 | NH1 | ARG | A | 1 | −23.354 | −7.761 | 29.451 | 1.00 | 164.47 | A |
| ATOM | 7 | NH2 | ARG | A | 1 | −25.082 | −7.656 | 27.948 | 1.00 | 164.47 | A |
| ATOM | 8 | C | ARG | A | 1 | −25.992 | −4.733 | 30.830 | 1.00 | 200.84 | A |
| ATOM | 9 | O | ARG | A | 1 | −25.498 | −5.373 | 29.901 | 1.00 | 200.84 | A |
| ATOM | 10 | N | ARG | A | 1 | −27.573 | −6.510 | 31.588 | 1.00 | 200.84 | A |
| ATOM | 11 | CA | ARG | A | 1 | −26.621 | −5.450 | 32.030 | 1.00 | 200.84 | A |
| ATOM | 12 | N | THR | A | 2 | −26.020 | −3.403 | 30.851 | 1.00 | 197.25 | A |
| ATOM | 13 | CA | THR | A | 2 | −25.460 | −2.602 | 29.762 | 1.00 | 197.25 | A |
| ATOM | 14 | CB | THR | A | 2 | −25.981 | −1.140 | 29.806 | 1.00 | 109.44 | A |
| ATOM | 15 | OG1 | THR | A | 2 | −27.413 | −1.134 | 29.776 | 1.00 | 108.18 | A |
| ATOM | 16 | CG2 | THR | A | 2 | −25.441 | −0.339 | 28.621 | 1.00 | 111.33 | A |
| ATOM | 17 | C | THR | A | 2 | −23.940 | −2.535 | 29.844 | 1.00 | 197.25 | A |
| ATOM | 18 | O | THR | A | 2 | −23.403 | −1.906 | 30.756 | 1.00 | 197.25 | A |
| ATOM | 19 | N | PRO | A | 3 | −23.222 | −3.191 | 28.909 | 1.00 | 200.84 | A |
| ATOM | 20 | CD | PRO | A | 3 | −23.593 | −4.120 | 27.823 | 1.00 | 99.78 | A |
| ATOM | 21 | CA | PRO | A | 3 | −21.767 | −3.083 | 29.028 | 1.00 | 200.84 | A |
| ATOM | 22 | CB | PRO | A | 3 | −21.275 | −3.640 | 27.694 | 1.00 | 98.02 | A |
| ATOM | 23 | CG | PRO | A | 3 | −22.248 | −4.759 | 27.454 | 1.00 | 97.89 | A |
| ATOM | 24 | C | PRO | A | 3 | −21.415 | −1.609 | 29.249 | 1.00 | 200.84 | A |
| ATOM | 25 | O | PRO | A | 3 | −20.477 | −1.295 | 29.983 | 1.00 | 200.84 | A |
| ATOM | 26 | N | SER | A | 4 | −22.198 | −0.720 | 28.626 | 1.00 | 133.96 | A |
| ATOM | 27 | CA | SER | A | 4 | −22.032 | 0.731 | 28.759 | 1.00 | 130.53 | A |
| ATOM | 28 | CB | SER | A | 4 | −20.551 | 1.124 | 28.863 | 1.00 | 109.88 | A |
| ATOM | 29 | OG | SER | A | 4 | −19.948 | 1.214 | 27.585 | 1.00 | 111.83 | A |
| ATOM | 30 | C | SER | A | 4 | −22.658 | 1.545 | 27.632 | 1.00 | 130.87 | A |
| ATOM | 31 | O | SER | A | 4 | −23.114 | 2.664 | 27.871 | 1.00 | 131.02 | A |
| ATOM | 32 | N | ASP | A | 5 | −22.672 | 0.982 | 26.420 | 1.00 | 59.92 | A |
| ATOM | 33 | CA | ASP | A | 5 | −23.201 | 1.648 | 25.217 | 1.00 | 60.03 | A |
| ATOM | 34 | CB | ASP | A | 5 | −24.033 | 2.876 | 25.616 | 1.00 | 142.51 | A |
| ATOM | 35 | CG | ASP | A | 5 | −24.553 | 3.649 | 24.431 | 1.00 | 142.51 | A |
| ATOM | 36 | OD1 | ASP | A | 5 | −25.094 | 4.755 | 24.642 | 1.00 | 142.51 | A |
| ATOM | 37 | OD2 | ASP | A | 5 | −24.432 | 3.155 | 23.294 | 1.00 | 141.07 | A |
| ATOM | 38 | C | ASP | A | 5 | −22.023 | 2.077 | 24.309 | 1.00 | 57.53 | A |
| ATOM | 39 | O | ASP | A | 5 | −22.187 | 2.277 | 23.106 | 1.00 | 61.04 | A |
| ATOM | 40 | N | LYS | A | 6 | −20.835 | 2.199 | 24.903 | 1.00 | 51.31 | A |
| ATOM | 41 | CA | LYS | A | 6 | −19.619 | 2.606 | 24.199 | 1.00 | 47.69 | A |
| ATOM | 42 | CB | LYS | A | 6 | −18.521 | 3.006 | 25.189 | 1.00 | 46.83 | A |
| ATOM | 43 | CG | LYS | A | 6 | −18.685 | 4.282 | 25.959 | 1.00 | 53.24 | A |
| ATOM | 44 | CD | LYS | A | 6 | −17.539 | 4.376 | 26.975 | 1.00 | 56.85 | A |
| ATOM | 45 | CE | LYS | A | 6 | −17.487 | 5.729 | 27.666 | 1.00 | 60.12 | A |
| ATOM | 46 | NZ | LYS | A | 6 | −17.213 | 6.816 | 26.677 | 1.00 | 61.31 | A |
| ATOM | 47 | C | LYS | A | 6 | −19.026 | 1.491 | 23.372 | 1.00 | 43.02 | A |
| ATOM | 48 | O | LYS | A | 6 | −18.896 | 0.373 | 23.853 | 1.00 | 46.90 | A |
| ATOM | 49 | N | PRO | A | 7 | −18.661 | 1.767 | 22.115 | 1.00 | 48.03 | A |
| ATOM | 50 | CD | PRO | A | 7 | −18.940 | 2.909 | 21.239 | 1.00 | 32.31 | A |
| ATOM | 51 | CA | PRO | A | 7 | −18.062 | 0.659 | 21.361 | 1.00 | 46.07 | A |
| ATOM | 52 | CB | PRO | A | 7 | −17.802 | 1.259 | 19.966 | 1.00 | 34.31 | A |
| ATOM | 53 | CG | PRO | A | 7 | −17.866 | 2.729 | 20.173 | 1.00 | 33.18 | A |
| ATOM | 54 | C | PRO | A | 7 | −16.774 | 0.277 | 22.101 | 1.00 | 44.13 | A |
| ATOM | 55 | O | PRO | A | 7 | −15.997 | 1.144 | 22.524 | 1.00 | 45.14 | A |
| ATOM | 56 | N | VAL | A | 8 | −16.568 | −1.021 | 22.286 | 1.00 | 30.79 | A |
| ATOM | 57 | CA | VAL | A | 8 | −15.397 | −1.489 | 23.002 | 1.00 | 31.95 | A |
| ATOM | 58 | CB | VAL | A | 8 | −15.672 | −1.495 | 24.526 | 1.00 | 35.54 | A |
| ATOM | 59 | CG1 | VAL | A | 8 | −16.868 | −2.394 | 24.832 | 1.00 | 36.12 | A |
| ATOM | 60 | CG2 | VAL | A | 8 | −14.445 | −1.969 | 25.291 | 1.00 | 38.24 | A |
| ATOM | 61 | C | VAL | A | 8 | −15.025 | −2.893 | 22.571 | 1.00 | 34.45 | A |
| ATOM | 62 | O | VAL | A | 8 | −15.851 | −3.627 | 22.021 | 1.00 | 33.69 | A |
| ATOM | 63 | N | ALA | A | 9 | −13.780 | −3.270 | 22.841 | 1.00 | 36.26 | A |
| ATOM | 64 | CA | ALA | A | 9 | −13.327 | −4.598 | 22.494 | 1.00 | 42.50 | A |
| ATOM | 65 | CB | ALA | A | 9 | −13.176 | −4.727 | 20.992 | 1.00 | 35.89 | A |
| ATOM | 66 | C | ALA | A | 9 | −12.018 | −4.921 | 23.157 | 1.00 | 43.48 | A |
| ATOM | 67 | O | ALA | A | 9 | −11.121 | −4.092 | 23.213 | 1.00 | 41.46 | A |
| ATOM | 68 | N | HIS | A | 10 | −11.924 | −6.132 | 23.681 | 1.00 | 35.22 | A |
| ATOM | 69 | CA | HIS | A | 10 | −10.703 | −6.607 | 24.294 | 1.00 | 36.09 | A |
| ATOM | 70 | CB | HIS | A | 10 | −10.793 | −6.569 | 25.811 | 1.00 | 38.90 | A |
| ATOM | 71 | CG | HIS | A | 10 | −9.537 | −7.019 | 26.496 | 1.00 | 40.39 | A |
| ATOM | 72 | CD2 | HIS | A | 10 | −9.205 | −8.205 | 27.060 | 1.00 | 38.12 | A |
| ATOM | 73 | ND1 | HIS | A | 10 | −8.426 | −6.210 | 26.621 | 1.00 | 40.53 | A |
| ATOM | 74 | CE1 | HIS | A | 10 | −7.464 | −6.880 | 27.232 | 1.00 | 40.88 | A |
| ATOM | 75 | NE2 | HIS | A | 10 | −7.912 | −8.092 | 27.509 | 1.00 | 38.36 | A |
| ATOM | 76 | C | HIS | A | 10 | −10.613 | −8.045 | 23.827 | 1.00 | 34.30 | A |
| ATOM | 77 | O | HIS | A | 10 | −11.260 | −8.933 | 24.394 | 1.00 | 32.46 | A |
| ATOM | 78 | N | VAL | A | 11 | −9.847 | −8.276 | 22.770 | 1.00 | 32.01 | A |
| ATOM | 79 | CA | VAL | A | 11 | −9.703 | −9.615 | 22.245 | 1.00 | 34.59 | A |
| ATOM | 80 | CB | VAL | A | 11 | −9.786 | −9.619 | 20.731 | 1.00 | 28.34 | A |
| ATOM | 81 | CG1 | VAL | A | 11 | −11.137 | −9.107 | 20.332 | 1.00 | 24.40 | A |
| ATOM | 82 | CG2 | VAL | A | 11 | −8.668 | −8.757 | 20.121 | 1.00 | 30.32 | A |
| ATOM | 83 | C | VAL | A | 11 | −8.389 | −10.205 | 22.709 | 1.00 | 33.20 | A |
| ATOM | 84 | O | VAL | A | 11 | −7.422 | −9.483 | 23.011 | 1.00 | 30.21 | A |
| ATOM | 65 | N | VAL | A | 12 | −8.352 | −11.525 | 22.775 | 1.00 | 46.19 | A |
| ATOM | 86 | CA | VAL | A | 12 | −7.173 | −12.194 | 23.268 | 1.00 | 44.36 | A |
| ATOM | 87 | CB | VAL | A | 12 | −7.526 | −12.855 | 24.601 | 1.00 | 40.90 | A |
| ATOM | 88 | CG1 | VAL | A | 12 | −8.400 | −14.076 | 24.349 | 1.00 | 34.70 | A |
| ATOM | 89 | CG2 | VAL | A | 12 | −6.272 | −13.199 | 25.359 | 1.00 | 44.37 | A |
| ATOM | 90 | C | VAL | A | 12 | −6.681 | −13.215 | 22.239 | 1.00 | 42.32 | A |
| ATOM | 91 | O | VAL | A | 12 | −7.433 | −13.612 | 21.340 | 1.00 | 42.16 | A |
| ATOM | 92 | N | ALA | A | 13 | −5.420 | −13.626 | 22.360 | 1.00 | 53.96 | A |
| ATOM | 93 | CA | ALA | A | 13 | −4.827 | −14.584 | 21.425 | 1.00 | 53.63 | A |
| ATOM | 94 | CB | ALA | A | 13 | −3.314 | −14.562 | 21.554 | 1.00 | 46.20 | A |
| ATOM | 95 | C | ALA | A | 13 | −5.331 | −16.012 | 21.592 | 1.00 | 57.61 | A |
| ATOM | 96 | O | ALA | A | 13 | −5.512 | −16.498 | 22.709 | 1.00 | 55.12 | A |
| ATOM | 97 | N | ASN | A | 14 | −5.543 | −16.683 | 20.465 | 1.00 | 44.46 | A |
| ATOM | 98 | CA | ASN | A | 14 | −6.017 | −18.058 | 20.455 | 1.00 | 45.58 | A |
| ATOM | 99 | CB | ASN | A | 14 | −6.690 | −18.348 | 19.124 | 1.00 | 52.51 | A |
| ATOM | 100 | CG | ASN | A | 14 | −7.190 | −19.769 | 19.025 | 1.00 | 54.50 | A |
| ATOM | 101 | OD1 | ASN | A | 14 | −7.686 | −20.182 | 17.976 | 1.00 | 54.08 | A |
| ATOM | 102 | ND2 | ASN | A | 14 | −7.071 | −20.526 | 20.114 | 1.00 | 56.62 | A |
| ATOM | 103 | C | ASN | A | 14 | −4.831 | −18.994 | 20.641 | 1.00 | 48.46 | A |
| ATOM | 104 | O | ASN | A | 14 | −3.945 | −19.045 | 19.787 | 1.00 | 48.98 | A |
| ATOM | 105 | N | PRO | A | 15 | −4.801 | −19.755 | 21.751 | 1.00 | 71.99 | A |
| ATOM | 106 | CD | PRO | A | 15 | −5.772 | −19.803 | 22.854 | 1.00 | 35.03 | A |
| ATOM | 107 | CA | PRO | A | 15 | −3.687 | −20.679 | 22.002 | 1.00 | 75.59 | A |
| ATOM | 108 | CB | PRO | A | 15 | −3.839 | −21.035 | 23.483 | 1.00 | 39.37 | A |
| ATOM | 109 | CG | PRO | A | 15 | −4.879 | −20.071 | 24.022 | 1.00 | 37.64 | A |
| ATOM | 110 | C | PRO | A | 15 | −3.756 | −21.926 | 21.122 | 1.00 | 76.80 | A |
| ATOM | 111 | O | PRO | A | 15 | −2.750 | −22.603 | 20.912 | 1.00 | 78.83 | A |
| ATOM | 112 | N | GLN | A | 16 | −4.947 | −22.216 | 20.609 | 1.00 | 82.35 | A |
| ATOM | 113 | CA | GLN | A | 16 | −5.160 | −23.389 | 19.770 | 1.00 | 81.30 | A |
| ATOM | 114 | CB | GLN | A | 16 | −6.649 | −23.760 | 19.774 | 1.00 | 122.84 | A |
| ATOM | 115 | CG | GLN | A | 16 | −6.971 | −25.194 | 19.357 | 1.00 | 125.77 | A |
| ATOM | 116 | CD | GLN | A | 16 | −6.316 | −26.230 | 20.258 | 1.00 | 127.56 | A |
| ATOM | 117 | OE1 | GLN | A | 16 | −6.296 | −26.082 | 21.482 | 1.00 | 129.68 | A |
| ATOM | 118 | NE2 | GLN | A | 16 | −5.787 | −27.291 | 19.655 | 1.00 | 126.21 | A |
| ATOM | 119 | C | GLN | A | 16 | −4.678 | −23.171 | 18.334 | 1.00 | 77.97 | A |
| ATOM | 120 | O | GLN | A | 16 | −4.821 | −24.054 | 17.487 | 1.00 | 78.54 | A |
| ATOM | 121 | N | ALA | A | 17 | −4.106 | −22.001 | 18.059 | 1.00 | 85.13 | A |
| ATOM | 122 | CA | ALA | A | 17 | −3.609 | −21.700 | 16.715 | 1.00 | 88.89 | A |
| ATOM | 123 | CB | ALA | A | 17 | −4.318 | −20.483 | 16.151 | 1.00 | 56.63 | A |
| ATOM | 124 | C | ALA | A | 17 | −2.108 | −21.467 | 16.730 | 1.00 | 93.39 | A |
| ATOM | 125 | O | ALA | A | 17 | −1.632 | −20.378 | 16.427 | 1.00 | 95.03 | A |
| ATOM | 126 | N | GLU | A | 18 | −1.369 | −22.510 | 17.082 | 1.00 | 92.77 | A |
| ATOM | 127 | CA | GLU | A | 18 | 0.083 | −22.443 | 17.150 | 1.00 | 94.77 | A |
| ATOM | 128 | CB | GLU | A | 18 | 0.662 | −23.845 | 17.358 | 1.00 | 123.94 | A |
| ATOM | 129 | CG | GLU | A | 18 | 0.338 | −24.818 | 16.235 | 1.00 | 123.94 | A |
| ATOM | 130 | CD | GLU | A | 18 | 1.041 | −26.150 | 16.397 | 1.00 | 123.94 | A |
| ATOM | 131 | OE1 | GLU | A | 18 | 2.290 | −26.163 | 16.409 | 1.00 | 123.94 | A |
| ATOM | 132 | OE2 | GLU | A | 18 | 0.345 | −27.180 | 16.513 | 1.00 | 123.94 | A |
| ATOM | 133 | C | GLU | A | 18 | 0.708 | −21.826 | 15.904 | 1.00 | 94.43 | A |
| ATOM | 134 | O | GLU | A | 18 | 0.179 | −21.953 | 14.794 | 1.00 | 94.04 | A |
| ATOM | 135 | N | GLY | A | 19 | 1.837 | −21.151 | 16.110 | 1.00 | 80.24 | A |
| ATOM | 136 | CA | GLY | A | 19 | 2.554 | −20.534 | 15.013 | 1.00 | 77.73 | A |
| ATOM | 137 | C | GLY | A | 19 | 2.007 | −19.226 | 14.487 | 1.00 | 76.23 | A |
| ATOM | 138 | O | GLY | A | 19 | 2.663 | −18.584 | 13.672 | 1.00 | 76.24 | A |
| ATOM | 139 | N | GLN | A | 20 | 0.824 | −18.819 | 14.936 | 1.00 | 74.98 | A |
| ATOM | 140 | CA | GLN | A | 20 | 0.235 | −17.569 | 14.465 | 1.00 | 70.55 | A |
| ATOM | 141 | CB | GLN | A | 20 | −0.749 | −17.863 | 13.336 | 1.00 | 113.83 | A |
| ATOM | 142 | CG | GLN | A | 20 | −1.800 | −18.890 | 13.708 | 1.00 | 114.81 | A |
| ATOM | 143 | CD | GLN | A | 20 | −2.722 | −19.212 | 12.554 | 1.00 | 114.57 | A |
| ATOM | 144 | OE1 | GLN | A | 20 | −2.274 | −19.666 | 11.504 | 1.00 | 114.22 | A |
| ATOM | 145 | NE2 | GLN | A | 20 | −4.020 | −18.976 | 12.739 | 1.00 | 114.02 | A |
| ATOM | 146 | C | GLN | A | 20 | −0.463 | −16.786 | 15.574 | 1.00 | 65.29 | A |
| ATOM | 147 | O | GLN | A | 20 | −0.662 | −17.291 | 16.681 | 1.00 | 64.00 | A |
| ATOM | 148 | N | LEU | A | 21 | −0.833 | −15.546 | 15.271 | 1.00 | 58.00 | A |
| ATOM | 149 | CA | LEU | A | 21 | −1.507 | −14.690 | 16.240 | 1.00 | 54.46 | A |
| ATOM | 150 | CB | LEU | A | 21 | −0.740 | −13.375 | 16.406 | 1.00 | 49.22 | A |
| ATOM | 151 | CG | LEU | A | 21 | −1.324 | −12.305 | 17.334 | 1.00 | 48.00 | A |
| ATOM | 152 | CD1 | LEU | A | 21 | −1.741 | −12.912 | 18.651 | 1.00 | 45.30 | A |
| ATOM | 153 | CD2 | LEU | A | 21 | −0.277 | −11.224 | 17.555 | 1.00 | 50.04 | A |
| ATOM | 154 | C | LEU | A | 21 | −2.923 | −14.397 | 15.792 | 1.00 | 53.07 | A |
| ATOM | 155 | O | LEU | A | 21 | −3.160 | −13.442 | 15.061 | 1.00 | 48.39 | A |
| ATOM | 156 | N | GLN | A | 22 | −3.861 | −15.221 | 16.237 | 1.00 | 63.39 | A |
| ATOM | 157 | CA | GLN | A | 22 | −5.261 | −15.063 | 15.872 | 1.00 | 67.99 | A |
| ATOM | 158 | CB | GLN | A | 22 | −5.795 | −16.412 | 15.395 | 1.00 | 69.19 | A |
| ATOM | 159 | CG | GLN | A | 22 | −7.282 | −16.458 | 15.141 | 1.00 | 75.96 | A |
| ATOM | 160 | CD | GLN | A | 22 | −7.775 | −17.877 | 14.986 | 1.00 | 82.18 | A |
| ATOM | 161 | OE1 | GLN | A | 22 | −8.977 | −18.123 | 14.899 | 1.00 | 87.41 | A |
| ATOM | 162 | NE2 | GLN | A | 22 | −6.844 | −18.824 | 14.950 | 1.00 | 82.94 | A |
| ATOM | 163 | C | GLN | A | 22 | −6.065 | −14.550 | 17.075 | 1.00 | 65.38 | A |
| ATOM | 164 | O | GLN | A | 22 | −6.032 | −15.153 | 18.148 | 1.00 | 64.33 | A |
| ATOM | 165 | N | TRP | A | 23 | −6.789 | −13.446 | 16.899 | 1.00 | 46.36 | A |
| ATOM | 166 | CA | TRP | A | 23 | −7.565 | −12.864 | 17.995 | 1.00 | 40.24 | A |
| ATOM | 167 | CB | TRP | A | 23 | −7.700 | −11.347 | 17.822 | 1.00 | 38.15 | A |
| ATOM | 168 | CG | TRP | A | 23 | −6.413 | −10.604 | 17.656 | 1.00 | 38.67 | A |
| ATOM | 169 | CD2 | TRP | A | 23 | −5.406 | −10.392 | 18.656 | 1.00 | 36.78 | A |
| ATOM | 170 | CE2 | TRP | A | 23 | −4.375 | −9.640 | 18.059 | 1.00 | 38.40 | A |
| ATOM | 171 | CE3 | TRP | A | 23 | −5.276 | −10.769 | 19.999 | 1.00 | 36.47 | A |
| ATOM | 172 | CD1 | TRP | A | 23 | −5.966 | −9.990 | 16.533 | 1.00 | 38.14 | A |
| ATOM | 173 | NE1 | TRP | A | 23 | −4.744 | −9.405 | 16.764 | 1.00 | 37.23 | A |
| ATOM | 174 | CZ2 | TRP | A | 23 | −3.227 | −9.256 | 18.754 | 1.00 | 40.46 | A |
| ATOM | 175 | CZ3 | TRP | A | 23 | −4.132 | −10.386 | 20.693 | 1.00 | 40.91 | A |
| ATOM | 176 | CH2 | TRP | A | 23 | −3.124 | −9.638 | 20.067 | 1.00 | 38.72 | A |
| ATOM | 177 | C | TRP | A | 23 | −8.974 | −13.432 | 18.135 | 1.00 | 40.74 | A |
| ATOM | 178 | O | TRP | A | 23 | −9.668 | −13.643 | 17.146 | 1.00 | 42.02 | A |
| ATOM | 179 | N | LEU | A | 24 | −9.399 | −13.677 | 19.370 | 1.00 | 35.76 | A |
| ATOM | 180 | CA | LEU | A | 24 | −10.754 | −14.162 | 19.606 | 1.00 | 33.45 | A |
| ATOM | 181 | CB | LEU | A | 24 | −10.801 | −15.638 | 20.018 | 1.00 | 75.48 | A |
| ATOM | 182 | CG | LEU | A | 24 | −9.618 | −16.569 | 19.826 | 1.00 | 76.21 | A |
| ATOM | 183 | CD1 | LEU | A | 24 | −8.550 | −16.204 | 20.825 | 1.00 | 77.83 | A |
| ATOM | 184 | CD2 | LEU | A | 24 | −10.066 | −17.993 | 20.048 | 1.00 | 71.32 | A |
| ATOM | 185 | C | LEU | A | 24 | −11.382 | −13.350 | 20.711 | 1.00 | 40.31 | A |
| ATOM | 186 | O | LEU | A | 24 | −10.693 | −12.738 | 21.527 | 1.00 | 37.29 | A |
| ATOM | 187 | N | ASN | A | 25 | −12.702 | −13.364 | 20.731 | 1.00 | 39.29 | A |
| ATOM | 188 | CA | ASN | A | 25 | −13.457 | −12.635 | 21.723 | 1.00 | 40.73 | A |
| ATOM | 189 | CB | ASN | A | 25 | −14.317 | −11.597 | 21.027 | 1.00 | 33.85 | A |
| ATOM | 190 | CG | ASN | A | 25 | −15.489 | −12.225 | 20.312 | 1.00 | 35.42 | A |
| ATOM | 191 | OD1 | ASN | A | 25 | −16.647 | −12.030 | 20.704 | 1.00 | 39.41 | A |
| ATOM | 192 | ND2 | ASN | A | 25 | −15.198 | −13.010 | 19.269 | 1.00 | 31.82 | A |
| ATOM | 193 | C | ASN | A | 25 | −14.376 | −13.596 | 22.482 | 1.00 | 41.26 | A |
| ATOM | 194 | O | ASN | A | 25 | −15.096 | −13.187 | 23.391 | 1.00 | 43.62 | A |
| ATOM | 195 | N | ARG | A | 26 | −14.370 | −14.866 | 22.101 | 1.00 | 51.77 | A |
| ATOM | 196 | CA | ARG | A | 26 | −15.227 | −15.849 | 22.749 | 1.00 | 53.05 | A |
| ATOM | 197 | CB | ARG | A | 26 | −15.624 | −16.938 | 21.749 | 1.00 | 43.00 | A |
| ATOM | 198 | CG | ARG | A | 26 | −14.460 | −17.626 | 21.065 | 1.00 | 43.91 | A |
| ATOM | 199 | CD | ARG | A | 26 | −14.930 | −18.861 | 20.344 | 1.00 | 47.97 | A |
| ATOM | 200 | NE | ARG | A | 26 | −13.844 | −19.554 | 19.653 | 1.00 | 49.11 | A |
| ATOM | 201 | CZ | ARG | A | 26 | −13.396 | −19.224 | 18.447 | 1.00 | 49.67 | A |
| ATOM | 202 | NH1 | ARG | A | 26 | −13.946 | −18.205 | 17.794 | 1.00 | 50.15 | A |
| ATOM | 203 | NH2 | ARG | A | 26 | −12.410 | −19.916 | 17.895 | 1.00 | 48.70 | A |
| ATOM | 204 | C | ARG | A | 26 | −14.595 | −16.504 | 23.970 | 1.00 | 51.11 | A |
| ATOM | 205 | O | ARG | A | 26 | −14.641 | −17.729 | 24.111 | 1.00 | 50.82 | A |
| ATOM | 206 | N | ARG | A | 27 | −14.011 | −15.699 | 24.854 | 1.00 | 34.32 | A |
| ATOM | 207 | CA | ARG | A | 27 | −13.382 | −16.238 | 26.048 | 1.00 | 35.25 | A |
| ATOM | 208 | CB | ARG | A | 27 | −11.867 | −16.241 | 25.883 | 1.00 | 39.05 | A |
| ATOM | 209 | CG | ARG | A | 27 | −11.385 | −17.198 | 24.836 | 1.00 | 40.47 | A |
| ATOM | 210 | CD | ARG | A | 27 | −11.958 | −18.577 | 25.082 | 1.00 | 42.84 | A |
| ATOM | 211 | NE | ARG | A | 27 | −11.612 | −19.542 | 24.039 | 1.00 | 46.23 | A |
| ATOM | 212 | CZ | ARG | A | 27 | −10.371 | −19.931 | 23.764 | 1.00 | 49.84 | A |
| ATOM | 213 | NH1 | ARG | A | 27 | −10.160 | −20.814 | 22.795 | 1.00 | 50.71 | A |
| ATOM | 214 | NH2 | ARG | A | 27 | −9.343 | −19.436 | 24.458 | 1.00 | 48.07 | A |
| ATOM | 215 | C | ARG | A | 27 | −13.757 | −15.528 | 27.343 | 1.00 | 35.71 | A |
| ATOM | 216 | O | ARG | A | 27 | −14.452 | −14.509 | 27.343 | 1.00 | 35.85 | A |
| ATOM | 217 | N | ALA | A | 28 | −13.273 | −16.097 | 28.440 | 1.00 | 66.51 | A |
| ATOM | 218 | CA | ALA | A | 28 | −13.502 | −15.616 | 29.793 | 1.00 | 65.41 | A |
| ATOM | 219 | CB | ALA | A | 28 | −12.330 | −16.019 | 30.675 | 1.00 | 109.51 | A |
| ATOM | 220 | C | ALA | A | 28 | −13.753 | −14.131 | 29.936 | 1.00 | 65.07 | A |
| ATOM | 221 | O | ALA | A | 28 | −14.893 | −13.677 | 29.953 | 1.00 | 66.09 | A |
| ATOM | 222 | N | ASN | A | 29 | −12.688 | −13.358 | 30.050 | 1.00 | 35.16 | A |
| ATOM | 223 | CA | ASN | A | 29 | −12.858 | −11.930 | 30.237 | 1.00 | 34.33 | A |
| ATOM | 224 | CB | ASN | A | 29 | −12.047 | −11.493 | 31.455 | 1.00 | 43.38 | A |
| ATOM | 225 | CG | ASN | A | 29 | −12.523 | −12.162 | 32.751 | 1.00 | 43.44 | A |
| ATOM | 226 | OD1 | ASN | A | 29 | −13.506 | −11.742 | 33.360 | 1.00 | 49.30 | A |
| ATOM | 227 | ND2 | ASN | A | 29 | −11.826 | −13.207 | 33.167 | 1.00 | 40.45 | A |
| ATOM | 228 | C | ASN | A | 29 | −12.453 | −11.126 | 29.002 | 1.00 | 32.67 | A |
| ATOM | 229 | O | ASN | A | 29 | −11.540 | −10.304 | 29.050 | 1.00 | 30.39 | A |
| ATOM | 230 | N | ALA | A | 30 | −13.150 | −11.369 | 27.896 | 1.00 | 46.08 | A |
| ATOM | 231 | CA | ALA | A | 30 | −12.877 | −10.684 | 26.644 | 1.00 | 40.90 | A |
| ATOM | 232 | CB | ALA | A | 30 | −12.545 | −11.701 | 25.566 | 1.00 | 30.84 | A |
| ATOM | 233 | C | ALA | A | 30 | −14.094 | −9.858 | 26.246 | 1.00 | 42.30 | A |
| ATOM | 234 | O | ALA | A | 30 | −15.170 | −10.012 | 26.828 | 1.00 | 46.45 | A |
| ATOM | 235 | N | LEU | A | 31 | −13.928 | −8.982 | 25.262 | 1.00 | 46.03 | A |
| ATOM | 236 | CA | LEU | A | 31 | −15.032 | −8.133 | 24.839 | 1.00 | 45.50 | A |
| ATOM | 237 | CB | LEU | A | 31 | −14.995 | −6.804 | 25.587 | 1.00 | 27.52 | A |
| ATOM | 238 | CG | LEU | A | 31 | −15.337 | −6.709 | 27.068 | 1.00 | 30.40 | A |
| ATOM | 239 | CD1 | LEU | A | 31 | −14.889 | −5.348 | 27.602 | 1.00 | 28.02 | A |
| ATOM | 240 | CD2 | LEU | A | 31 | −16.825 | −6.900 | 27.250 | 1.00 | 29.87 | A |
| ATOM | 241 | C | LEU | A | 31 | −15.039 | −7.823 | 23.361 | 1.00 | 44.76 | A |
| ATOM | 242 | O | LEU | A | 31 | −13.987 | −7.738 | 22.735 | 1.00 | 45.57 | A |
| ATOM | 243 | N | LEU | A | 32 | −16.244 | −7.648 | 22.826 | 1.00 | 40.02 | A |
| ATOM | 244 | CA | LEU | A | 32 | −16.457 | −7.287 | 21.436 | 1.00 | 39.81 | A |
| ATOM | 245 | CB | LEU | A | 32 | −16.271 | −8.484 | 20.515 | 1.00 | 35.94 | A |
| ATOM | 246 | CG | LEU | A | 32 | −16.407 | −8.091 | 19.036 | 1.00 | 34.52 | A |
| ATOM | 247 | CD1 | LEU | A | 32 | −15.254 | −7.162 | 18.642 | 1.00 | 33.50 | A |
| ATOM | 248 | CD2 | LEU | A | 32 | −16.419 | −9.335 | 18.158 | 1.00 | 34.84 | A |
| ATOM | 249 | C | LEU | A | 32 | −17.900 | −6.812 | 21.396 | 1.00 | 39.45 | A |
| ATOM | 250 | O | LEU | A | 32 | −18.807 | −7.577 | 21.058 | 1.00 | 45.32 | A |
| ATOM | 251 | N | ALA | A | 33 | −18.122 | −5.542 | 21.733 | 1.00 | 43.98 | A |
| ATOM | 252 | CA | ALA | A | 33 | −19.480 | −5.030 | 21.770 | 1.00 | 44.50 | A |
| ATOM | 253 | CB | ALA | A | 33 | −19.991 | −5.087 | 23.186 | 1.00 | 14.30 | A |
| ATOM | 254 | C | ALA | A | 33 | −19.745 | −3.653 | 21.215 | 1.00 | 46.29 | A |
| ATOM | 255 | O | ALA | A | 33 | −18.839 | −2.913 | 20.856 | 1.00 | 50.47 | A |
| ATOM | 256 | N | ASN | A | 34 | −21.037 | −3.342 | 21.163 | 1.00 | 44.70 | A |
| ATOM | 257 | CA | ASN | A | 34 | −21.542 | −2.074 | 20.695 | 1.00 | 41.89 | A |
| ATOM | 258 | CB | ASN | A | 34 | −21.250 | −1.014 | 21.731 | 1.00 | 48.50 | A |
| ATOM | 259 | CG | ASN | A | 34 | −21.876 | −1.343 | 23.050 | 1.00 | 52.42 | A |
| ATOM | 260 | OD1 | ASN | A | 34 | −23.067 | −1.638 | 23.122 | 1.00 | 54.90 | A |
| ATOM | 261 | ND2 | ASN | A | 34 | −21.083 | −1.306 | 24.106 | 1.00 | 47.83 | A |
| ATOM | 262 | C | ASN | A | 34 | −21.059 | −1.617 | 19.341 | 1.00 | 42.64 | A |
| ATOM | 263 | O | ASN | A | 34 | −20.737 | −0.445 | 19.152 | 1.00 | 45.18 | A |
| ATOM | 264 | N | GLY | A | 35 | −21.003 | −2.537 | 18.389 | 1.00 | 41.76 | A |
| ATOM | 265 | CA | GLY | A | 35 | −20.614 | −2.137 | 17.060 | 1.00 | 36.35 | A |
| ATOM | 266 | C | GLY | A | 35 | −19.269 | −2.539 | 16.526 | 1.00 | 36.70 | A |
| ATOM | 267 | O | GLY | A | 35 | −19.152 | −2.771 | 15.320 | 1.00 | 38.19 | A |
| ATOM | 268 | N | VAL | A | 36 | −18.239 | −2.612 | 17.362 | 1.00 | 39.33 | A |
| ATOM | 269 | CA | VAL | A | 36 | −16.949 | −2.972 | 16.795 | 1.00 | 40.66 | A |
| ATOM | 270 | CB | VAL | A | 36 | −15.766 | −2.653 | 17.771 | 1.00 | 37.15 | A |
| ATOM | 271 | CG1 | VAL | A | 36 | −16.303 | −2.242 | 19.105 | 1.00 | 35.18 | A |
| ATOM | 272 | CG2 | VAL | A | 36 | −14.823 | −3.826 | 17.890 | 1.00 | 37.55 | A |
| ATOM | 273 | C | VAL | A | 36 | −16.980 | −4.427 | 16.365 | 1.00 | 44.81 | A |
| ATOM | 274 | O | VAL | A | 36 | −17.535 | −5.287 | 17.047 | 1.00 | 45.77 | A |
| ATOM | 275 | N | GLU | A | 37 | −16.408 | −4.686 | 15.198 | 1.00 | 43.54 | A |
| ATOM | 276 | CA | GLU | A | 37 | −16.377 | −6.026 | 14.640 | 1.00 | 46.14 | A |
| ATOM | 277 | CB | GLU | A | 37 | −17.008 | −6.009 | 13.254 | 1.00 | 54.70 | A |
| ATOM | 278 | CG | GLU | A | 37 | −18.199 | −5.081 | 13.174 | 1.00 | 61.73 | A |
| ATOM | 279 | CD | GLU | A | 37 | −18.630 | −4.783 | 11.753 | 1.00 | 64.70 | A |
| ATOM | 280 | OE1 | GLU | A | 37 | −17.756 | −4.794 | 10.848 | 1.00 | 68.13 | A |
| ATOM | 281 | OE2 | GLU | A | 37 | −19.840 | −4.517 | 11.550 | 1.00 | 65.26 | A |
| ATOM | 282 | C | GLU | A | 37 | −14.952 | −6.548 | 14.544 | 1.00 | 45.03 | A |
| ATOM | 283 | O | GLU | A | 37 | −13.987 | −5.785 | 14.472 | 1.00 | 47.49 | A |
| ATOM | 284 | N | LEU | A | 38 | −14.831 | −7.865 | 14.577 | 1.00 | 56.99 | A |
| ATOM | 285 | CA | LEU | A | 38 | −13.541 | −8.502 | 14.451 | 1.00 | 59.03 | A |
| ATOM | 286 | CB | LEU | A | 38 | −13.353 | −9.531 | 15.565 | 1.00 | 36.82 | A |
| ATOM | 287 | CG | LEU | A | 38 | −11.990 | −10.237 | 15.590 | 1.00 | 34.73 | A |
| ATOM | 288 | CD1 | LEU | A | 38 | −10.878 | −9.196 | 15.482 | 1.00 | 33.11 | A |
| ATOM | 289 | CD2 | LEU | A | 38 | −11.842 | −11.072 | 16.859 | 1.00 | 31.60 | A |
| ATOM | 290 | C | LEU | A | 38 | −13.650 | −9.156 | 13.078 | 1.00 | 59.30 | A |
| ATOM | 291 | O | LEU | A | 38 | −14.318 | −10.171 | 12.905 | 1.00 | 52.91 | A |
| ATOM | 292 | N | ARG | A | 39 | −13.014 | −8.537 | 12.094 | 1.00 | 42.84 | A |
| ATOM | 293 | CA | ARG | A | 39 | −13.063 | −9.000 | 10.709 | 1.00 | 49.37 | A |
| ATOM | 294 | CB | ARG | A | 39 | −13.799 | −7.953 | 9.884 | 1.00 | 50.15 | A |
| ATOM | 295 | CG | ARG | A | 39 | −14.177 | −8.376 | 8.509 | 1.00 | 57.85 | A |
| ATOM | 296 | CD | ARG | A | 39 | −15.142 | −7.372 | 7.924 | 1.00 | 66.46 | A |
| ATOM | 297 | NE | ARG | A | 39 | −15.754 | −7.883 | 6.705 | 1.00 | 72.99 | A |
| ATOM | 298 | CZ | ARG | A | 39 | −16.815 | −7.337 | 6.122 | 1.00 | 73.79 | A |
| ATOM | 299 | NH1 | ARG | A | 39 | −17.384 | −6.253 | 6.646 | 1.00 | 72.27 | A |
| ATOM | 300 | NH2 | ARG | A | 39 | −17.317 | −7.883 | 5.024 | 1.00 | 76.09 | A |
| ATOM | 301 | C | ARG | A | 39 | −11.667 | −9.224 | 10.150 | 1.00 | 51.42 | A |
| ATOM | 302 | O | ARG | A | 39 | −10.801 | −8.369 | 10.268 | 1.00 | 54.65 | A |
| ATOM | 303 | N | ASP | A | 40 | −11.447 | −10.382 | 9.551 | 1.00 | 52.29 | A |
| ATOM | 304 | CA | ASP | A | 40 | −10.149 | −10.715 | 8.976 | 1.00 | 50.24 | A |
| ATOM | 305 | CB | ASP | A | 40 | −9.925 | −9.917 | 7.684 | 1.00 | 114.16 | A |
| ATOM | 306 | CG | ASP | A | 40 | −11.043 | −10.114 | 6.669 | 1.00 | 116.30 | A |
| ATOM | 307 | OD1 | ASP | A | 40 | −11.341 | −11.280 | 6.332 | 1.00 | 113.18 | A |
| ATOM | 308 | OD2 | ASP | A | 40 | −11.623 | −9.106 | 6.204 | 1.00 | 116.42 | A |
| ATOM | 309 | C | ASP | A | 40 | −9.012 | −10.444 | 9.966 | 1.00 | 46.37 | A |
| ATOM | 310 | O | ASP | A | 40 | −7.927 | −9.986 | 9.591 | 1.00 | 45.60 | A |
| ATOM | 311 | N | ASN | A | 41 | −9.273 | −10.724 | 11.238 | 1.00 | 55.36 | A |
| ATOM | 312 | CA | ASN | A | 41 | −8.293 | −10.541 | 12.316 | 1.00 | 51.11 | A |
| ATOM | 313 | CB | ASN | A | 41 | −6.996 | −11.278 | 11.999 | 1.00 | 48.73 | A |
| ATOM | 314 | CG | ASN | A | 41 | −6.260 | −11.701 | 13.252 | 1.00 | 43.42 | A |
| ATOM | 315 | OD1 | ASN | A | 41 | −6.835 | −12.365 | 14.134 | 1.00 | 44.76 | A |
| ATOM | 316 | ND2 | ASN | A | 41 | −4.987 | −11.332 | 13.345 | 1.00 | 39.52 | A |
| ATOM | 317 | C | ASN | A | 41 | −7.965 | −9.095 | 12.649 | 1.00 | 54.42 | A |
| ATOM | 318 | O | ASN | A | 41 | −6.886 | −8.799 | 13.162 | 1.00 | 55.93 | A |
| ATOM | 319 | N | GLN | A | 42 | −8.901 | −8.199 | 12.359 | 1.00 | 62.44 | A |
| ATOM | 320 | CA | GLN | A | 42 | −8.708 | −6.785 | 12.638 | 1.00 | 60.36 | A |
| ATOM | 321 | CB | GLN | A | 42 | −8.444 | −6.018 | 11.348 | 1.00 | 48.14 | A |
| ATOM | 322 | CG | GLN | A | 42 | −7.415 | −6.661 | 10.459 | 1.00 | 50.23 | A |
| ATOM | 323 | CD | GLN | A | 42 | −7.232 | −5.926 | 9.139 | 1.00 | 49.69 | A |
| ATOM | 324 | OE1 | GLN | A | 42 | −8.206 | −5.606 | 8.449 | 1.00 | 49.12 | A |
| ATOM | 325 | NE2 | GLN | A | 42 | −5.973 | −5.667 | 8.775 | 1.00 | 51.45 | A |
| ATOM | 326 | C | GLN | A | 42 | −9.966 | −6.239 | 13.286 | 1.00 | 59.25 | A |
| ATOM | 327 | O | GLN | A | 42 | −11.073 | −6.701 | 13.006 | 1.00 | 59.59 | A |
| ATOM | 328 | N | LEU | A | 43 | −9.796 | −5.264 | 14.167 | 1.00 | 39.12 | A |
| ATOM | 329 | CA | LEU | A | 43 | −10.944 | −4.660 | 14.807 | 1.00 | 37.70 | A |
| ATOM | 330 | CB | LEU | A | 43 | −10.549 | −4.089 | 16.165 | 1.00 | 29.32 | A |
| ATOM | 331 | CG | LEU | A | 43 | −10.082 | −5.131 | 17.180 | 1.00 | 35.48 | A |
| ATOM | 332 | CD1 | LEU | A | 43 | −9.511 | −4.441 | 18.410 | 1.00 | 35.57 | A |
| ATOM | 333 | CD2 | LEU | A | 43 | −11.241 | −6.016 | 17.544 | 1.00 | 31.87 | A |
| ATOM | 334 | C | LEU | A | 43 | −11.444 | −3.551 | 13.877 | 1.00 | 39.72 | A |
| ATOM | 335 | O | LEU | A | 43 | −10.650 | −2.830 | 13.280 | 1.00 | 36.34 | A |
| ATOM | 336 | N | VAL | A | 44 | −12.757 | −3.427 | 13.739 | 1.00 | 44.00 | A |
| ATOM | 337 | CA | VAL | A | 44 | −13.316 | −2.408 | 12.874 | 1.00 | 43.68 | A |
| ATOM | 338 | CB | VAL | A | 44 | −14.135 | −3.035 | 11.749 | 1.00 | 46.83 | A |
| ATOM | 339 | CG1 | VAL | A | 44 | −14.553 | −1.967 | 10.763 | 1.00 | 48.02 | A |
| ATOM | 340 | CG2 | VAL | A | 44 | −13.313 | −4.112 | 11.063 | 1.00 | 41.44 | A |
| ATOM | 341 | C | VAL | A | 44 | −14.198 | −1.435 | 13.650 | 1.00 | 42.69 | A |
| ATOM | 342 | O | VAL | A | 44 | −15.232 | −1.817 | 14.221 | 1.00 | 43.73 | A |
| ATOM | 343 | N | VAL | A | 45 | −13.778 | −0.171 | 13.649 | 1.00 | 32.56 | A |
| ATOM | 344 | CA | VAL | A | 45 | −14.480 | 0.892 | 14.350 | 1.00 | 38.30 | A |
| ATOM | 345 | CB | VAL | A | 45 | −13.603 | 2.151 | 14.391 | 1.00 | 54.43 | A |
| ATOM | 346 | CG1 | VAL | A | 45 | −14.405 | 3.341 | 14.869 | 1.00 | 57.00 | A |
| ATOM | 347 | CG2 | VAL | A | 45 | −12.435 | 1.912 | 15.319 | 1.00 | 57.83 | A |
| ATOM | 348 | C | VAL | A | 45 | −15.837 | 1.203 | 13.711 | 1.00 | 40.09 | A |
| ATOM | 349 | O | VAL | A | 45 | −15.949 | 1.350 | 12.502 | 1.00 | 39.54 | A |
| ATOM | 350 | N | PRO | A | 46 | −16.887 | 1.311 | 14.534 | 1.00 | 41.97 | A |
| ATOM | 351 | CD | PRO | A | 46 | −16.909 | 0.906 | 15.954 | 1.00 | 38.18 | A |
| ATOM | 352 | CA | PRO | A | 46 | −18.239 | 1.598 | 14.053 | 1.00 | 40.82 | A |
| ATOM | 353 | CB | PRO | A | 46 | −19.102 | 0.810 | 15.021 | 1.00 | 37.54 | A |
| ATOM | 354 | CG | PRO | A | 46 | −18.377 | 1.071 | 16.326 | 1.00 | 38.24 | A |
| ATOM | 355 | C | PRO | A | 46 | −18.644 | 3.070 | 14.001 | 1.00 | 44.26 | A |
| ATOM | 356 | O | PRO | A | 46 | −19.725 | 3.390 | 13.522 | 1.00 | 46.72 | A |
| ATOM | 357 | N | SER | A | 47 | −17.805 | 3.972 | 14.490 | 1.00 | 57.78 | A |
| ATOM | 358 | CA | SER | A | 47 | −18.181 | 5.379 | 14.459 | 1.00 | 54.90 | A |
| ATOM | 359 | CB | SER | A | 47 | −19.234 | 5.650 | 15.532 | 1.00 | 63.77 | A |
| ATOM | 360 | OG | SER | A | 47 | −18.721 | 5.351 | 16.816 | 1.00 | 64.98 | A |
| ATOM | 361 | C | SER | A | 47 | −17.023 | 6.356 | 14.639 | 1.00 | 51.59 | A |
| ATOM | 362 | O | SER | A | 47 | −15.974 | 5.995 | 15.178 | 1.00 | 48.73 | A |
| ATOM | 363 | N | GLU | A | 48 | −17.226 | 7.594 | 14.177 | 1.00 | 51.64 | A |
| ATOM | 364 | CA | GLU | A | 48 | −16.230 | 8.662 | 14.297 | 1.00 | 56.13 | A |
| ATOM | 365 | CB | GLU | A | 48 | −16.804 | 9.996 | 13.813 | 1.00 | 173.15 | A |
| ATOM | 366 | CG | GLU | A | 48 | −17.350 | 10.009 | 12.399 | 1.00 | 177.89 | A |
| ATOM | 367 | CD | GLU | A | 48 | −16.274 | 10.201 | 11.351 | 1.00 | 182.24 | A |
| ATOM | 368 | OE1 | GLU | A | 48 | −15.453 | 9.281 | 11.160 | 1.00 | 183.98 | A |
| ATOM | 369 | OE2 | GLU | A | 48 | −16.248 | 11.280 | 10.721 | 1.00 | 180.16 | A |
| ATOM | 370 | C | GLU | A | 48 | −15.939 | 8.792 | 15.788 | 1.00 | 54.72 | A |
| ATOM | 371 | O | GLU | A | 48 | −16.815 | 8.533 | 16.617 | 1.00 | 60.51 | A |
| ATOM | 372 | N | GLY | A | 49 | −14.721 | 9.185 | 16.137 | 1.00 | 35.48 | A |
| ATOM | 373 | CA | GLY | A | 49 | −14.419 | 9.354 | 17.543 | 1.00 | 33.14 | A |
| ATOM | 374 | C | GLY | A | 49 | −12.995 | 9.137 | 18.002 | 1.00 | 35.15 | A |
| ATOM | 375 | O | GLY | A | 49 | −12.124 | 8.683 | 17.251 | 1.00 | 37.99 | A |
| ATOM | 376 | N | LEU | A | 50 | −12.777 | 9.485 | 19.261 | 1.00 | 43.28 | A |
| ATOM | 377 | CA | LEU | A | 50 | −11.490 | 9.348 | 19.914 | 1.00 | 44.48 | A |
| ATOM | 378 | CB | LEU | A | 50 | −11.392 | 10.407 | 21.017 | 1.00 | 33.10 | A |
| ATOM | 379 | CG | LEU | A | 50 | −10.360 | 11.527 | 20.919 | 1.00 | 34.23 | A |
| ATOM | 380 | CD1 | LEU | A | 50 | −10.056 | 11.856 | 19.479 | 1.00 | 31.04 | A |
| ATOM | 381 | CD2 | LEU | A | 50 | −10.887 | 12.726 | 21.669 | 1.00 | 34.62 | A |
| ATOM | 382 | C | LEU | A | 50 | −11.473 | 7.944 | 20.512 | 1.00 | 40.86 | A |
| ATOM | 383 | O | LEU | A | 50 | −12.417 | 7.541 | 21.183 | 1.00 | 39.58 | A |
| ATOM | 384 | N | TYR | A | 51 | −10.414 | 7.190 | 20.265 | 1.00 | 43.46 | A |
| ATOM | 385 | CA | TYR | A | 51 | −10.322 | 5.829 | 20.798 | 1.00 | 42.11 | A |
| ATOM | 386 | CB | TYR | A | 51 | −10.456 | 4.787 | 19.681 | 1.00 | 36.04 | A |
| ATOM | 387 | CG | TYR | A | 51 | −11.838 | 4.612 | 19.104 | 1.00 | 36.14 | A |
| ATOM | 388 | CD1 | TYR | A | 51 | −12.386 | 5.568 | 18.257 | 1.00 | 37.00 | A |
| ATOM | 389 | CE1 | TYR | A | 51 | −13.653 | 5.397 | 17.710 | 1.00 | 36.56 | A |
| ATOM | 390 | CD2 | TYR | A | 51 | −12.593 | 3.479 | 19.398 | 1.00 | 37.70 | A |
| ATOM | 391 | CE2 | TYR | A | 51 | −13.861 | 3.296 | 18.863 | 1.00 | 35.97 | A |
| ATOM | 392 | CZ | TYR | A | 51 | −14.384 | 4.254 | 18.018 | 1.00 | 35.67 | A |
| ATOM | 393 | OH | TYR | A | 51 | −15.631 | 4.060 | 17.472 | 1.00 | 35.90 | A |
| ATOM | 394 | C | TYR | A | 51 | −9.019 | 5.528 | 21.515 | 1.00 | 42.29 | A |
| ATOM | 395 | O | TYR | A | 51 | −7.940 | 5.972 | 21.104 | 1.00 | 43.18 | A |
| ATOM | 396 | N | LEU | A | 52 | −9.119 | 4.759 | 22.589 | 1.00 | 40.38 | A |
| ATOM | 397 | CA | LEU | A | 52 | −7.928 | 4.347 | 23.311 | 1.00 | 42.03 | A |
| ATOM | 398 | CB | LEU | A | 52 | −8.200 | 4.229 | 24.812 | 1.00 | 38.33 | A |
| ATOM | 399 | CG | LEU | A | 52 | −7.100 | 3.529 | 25.617 | 1.00 | 34.22 | A |
| ATOM | 400 | CD1 | LEU | A | 52 | −5.758 | 4.169 | 25.340 | 1.00 | 36.25 | A |
| ATOM | 401 | CD2 | LEU | A | 52 | −7.441 | 3.601 | 27.090 | 1.00 | 28.55 | A |
| ATOM | 402 | C | LEU | A | 52 | −7.633 | 2.983 | 22.705 | 1.00 | 43.56 | A |
| ATOM | 403 | O | LEU | A | 52 | −8.486 | 2.100 | 22.709 | 1.00 | 44.13 | A |
| ATOM | 404 | N | ILE | A | 53 | −6.443 | 2.832 | 22.143 | 1.00 | 40.95 | A |
| ATOM | 405 | CA | ILE | A | 53 | −6.048 | 1.579 | 21.521 | 1.00 | 37.55 | A |
| ATOM | 406 | CB | ILE | A | 53 | −5.605 | 1.800 | 20.069 | 1.00 | 47.46 | A |
| ATOM | 407 | CG2 | ILE | A | 53 | −5.262 | 0.481 | 19.416 | 1.00 | 48.10 | A |
| ATOM | 408 | CG1 | ILE | A | 53 | −6.716 | 2.475 | 19.285 | 1.00 | 46.35 | A |
| ATOM | 409 | CD1 | ILE | A | 53 | −6.323 | 2.754 | 17.864 | 1.00 | 42.17 | A |
| ATOM | 410 | C | ILE | A | 53 | −4.870 | 1.018 | 22.286 | 1.00 | 37.61 | A |
| ATOM | 411 | O | ILE | A | 53 | −3.976 | 1.757 | 22.678 | 1.00 | 40.69 | A |
| ATOM | 412 | N | TYR | A | 54 | −4.857 | −0.287 | 22.500 | 1.00 | 43.80 | A |
| ATOM | 413 | CA | TYR | A | 54 | −3.745 | −0.887 | 23.226 | 1.00 | 40.93 | A |
| ATOM | 414 | CB | TYR | A | 54 | −3.957 | −0.718 | 24.729 | 1.00 | 39.58 | A |
| ATOM | 415 | CG | TYR | A | 54 | −5.167 | −1.459 | 25.243 | 1.00 | 39.58 | A |
| ATOM | 416 | CD1 | TYR | A | 54 | −5.075 | −2.792 | 25.641 | 1.00 | 39.58 | A |
| ATOM | 417 | CE1 | TYR | A | 54 | −6.194 | −3.492 | 26.065 | 1.00 | 39.58 | A |
| ATOM | 418 | CD2 | TYR | A | 54 | −6.414 | −0.843 | 25.285 | 1.00 | 39.58 | A |
| ATOM | 419 | CE2 | TYR | A | 54 | −7.544 | −1.537 | 25.708 | 1.00 | 39.58 | A |
| ATOM | 420 | CZ | TYR | A | 54 | −7.425 | −2.859 | 26.093 | 1.00 | 39.58 | A |
| ATOM | 421 | OH | TYR | A | 54 | −8.538 | −3.554 | 26.480 | 1.00 | 39.58 | A |
| ATOM | 422 | C | TYR | A | 54 | −3.554 | −2.363 | 22.887 | 1.00 | 39.52 | A |
| ATOM | 423 | O | TYR | A | 54 | −4.459 | −3.020 | 22.368 | 1.00 | 38.37 | A |
| ATOM | 424 | N | SER | A | 55 | −2.364 | −2.870 | 23.183 | 1.00 | 28.57 | A |
| ATOM | 425 | CA | SER | A | 55 | −2.063 | −4.259 | 22.909 | 1.00 | 28.35 | A |
| ATOM | 426 | CB | SER | A | 55 | −1.877 | −4.467 | 21.420 | 1.00 | 42.60 | A |
| ATOM | 427 | OG | SER | A | 55 | −1.363 | −5.756 | 21.183 | 1.00 | 49.16 | A |
| ATOM | 428 | C | SER | A | 55 | −0.817 | −4.730 | 23.620 | 1.00 | 31.33 | A |
| ATOM | 429 | O | SER | A | 55 | 0.097 | −3.947 | 23.870 | 1.00 | 28.47 | A |
| ATOM | 430 | N | GLN | A | 56 | −0.790 | −6.011 | 23.965 | 1.00 | 40.14 | A |
| ATOM | 431 | CA | GLN | A | 56 | 0.380 | −6.575 | 24.609 | 1.00 | 40.84 | A |
| ATOM | 432 | CB | GLN | A | 56 | 0.228 | −6.662 | 26.117 | 1.00 | 56.88 | A |
| ATOM | 433 | CG | GLN | A | 56 | 1.520 | −7.130 | 26.755 | 1.00 | 58.48 | A |
| ATOM | 434 | CD | GLN | A | 56 | 1.350 | −7.579 | 28.182 | 1.00 | 61.60 | A |
| ATOM | 435 | OE1 | GLN | A | 56 | 0.919 | −8.704 | 28.442 | 1.00 | 61.13 | A |
| ATOM | 436 | NE2 | GLN | A | 56 | 1.689 | −6.702 | 29.126 | 1.00 | 64.71 | A |
| ATOM | 437 | C | GLN | A | 56 | 0.658 | −7.967 | 24.096 | 1.00 | 43.32 | A |
| ATOM | 438 | O | GLN | A | 56 | −0.254 | −8.780 | 23.954 | 1.00 | 41.40 | A |
| ATOM | 439 | N | VAL | A | 57 | 1.926 | −8.231 | 23.804 | 1.00 | 44.64 | A |
| ATOM | 440 | CA | VAL | A | 57 | 2.331 | −9.546 | 23.345 | 1.00 | 39.01 | A |
| ATOM | 441 | CB | VAL | A | 57 | 2.850 | −9.528 | 21.897 | 1.00 | 41.66 | A |
| ATOM | 442 | CG1 | VAL | A | 57 | 1.784 | −8.991 | 20.980 | 1.00 | 42.86 | A |
| ATOM | 443 | CG2 | VAL | A | 57 | 4.112 | −8.700 | 21.807 | 1.00 | 38.59 | A |
| ATOM | 444 | C | VAL | A | 57 | 3.448 | −9.996 | 24.266 | 1.00 | 41.62 | A |
| ATOM | 445 | O | VAL | A | 57 | 4.172 | −9.173 | 24.827 | 1.00 | 42.73 | A |
| ATOM | 446 | N | LEU | A | 58 | 3.569 | −11.306 | 24.440 | 1.00 | 50.66 | A |
| ATOM | 447 | CA | LEU | A | 58 | 4.613 | −11.855 | 25.289 | 1.00 | 49.11 | A |
| ATOM | 448 | CB | LEU | A | 58 | 4.005 | −12.580 | 26.485 | 1.00 | 52.63 | A |
| ATOM | 449 | CG | LEU | A | 58 | 4.851 | −12.634 | 27.755 | 1.00 | 54.65 | A |
| ATOM | 450 | CD1 | LEU | A | 58 | 4.381 | −13.803 | 28.607 | 1.00 | 48.67 | A |
| ATOM | 451 | CD2 | LEU | A | 58 | 6.313 | −12.794 | 27.407 | 1.00 | 52.43 | A |
| ATOM | 452 | C | LEU | A | 58 | 5.420 | −12.836 | 24.456 | 1.00 | 52.08 | A |
| ATOM | 453 | O | LEU | A | 58 | 4.895 | −13.845 | 23.981 | 1.00 | 53.71 | A |
| ATOM | 454 | N | PHE | A | 59 | 6.693 | −12.521 | 24.258 | 1.00 | 48.71 | A |
| ATOM | 455 | CA | PHE | A | 59 | 7.558 | −13.398 | 23.488 | 1.00 | 49.61 | A |
| ATOM | 456 | CB | PHE | A | 59 | 8.549 | −12.595 | 22.639 | 1.00 | 61.42 | A |
| ATOM | 457 | CG | PHE | A | 59 | 7.910 | −11.779 | 21.562 | 1.00 | 57.91 | A |
| ATOM | 458 | CD1 | PHE | A | 59 | 7.757 | −10.409 | 21.714 | 1.00 | 56.35 | A |
| ATOM | 459 | CD2 | PHE | A | 59 | 7.481 | −12.379 | 20.387 | 1.00 | 59.87 | A |
| ATOM | 460 | CE1 | PHE | A | 59 | 7.187 | −9.643 | 20.710 | 1.00 | 62.27 | A |
| ATOM | 461 | CE2 | PHE | A | 59 | 6.910 | −11.628 | 19.374 | 1.00 | 62.98 | A |
| ATOM | 462 | CZ | PHE | A | 59 | 6.762 | −10.256 | 19.533 | 1.00 | 61.58 | A |
| ATOM | 463 | C | PHE | A | 59 | 8.333 | −14.310 | 24.435 | 1.00 | 53.72 | A |
| ATOM | 464 | O | PHE | A | 59 | 8.695 | −13.912 | 25.545 | 1.00 | 53.41 | A |
| ATOM | 465 | N | LYS | A | 60 | 8.580 | −15.535 | 23.982 | 1.00 | 86.80 | A |
| ATOM | 466 | CA | LYS | A | 60 | 9.321 | −16.512 | 24.766 | 1.00 | 88.15 | A |
| ATOM | 467 | CB | LYS | A | 60 | 8.367 | −17.418 | 25.539 | 1.00 | 67.86 | A |
| ATOM | 468 | CG | LYS | A | 60 | 9.060 | −18.534 | 26.304 | 1.00 | 74.05 | A |
| ATOM | 469 | CD | LYS | A | 60 | 8.041 | −19.423 | 26.991 | 1.00 | 82.26 | A |
| ATOM | 470 | CE | LYS | A | 60 | 8.706 | −20.563 | 27.737 | 1.00 | 85.01 | A |
| ATOM | 471 | NZ | LYS | A | 60 | 7.690 | −21.401 | 28.430 | 1.00 | 85.35 | A |
| ATOM | 472 | C | LYS | A | 60 | 10.166 | −17.360 | 23.842 | 1.00 | 87.66 | A |
| ATOM | 473 | O | LYS | A | 60 | 9.712 | −17.774 | 22.777 | 1.00 | 90.41 | A |
| ATOM | 474 | N | GLY | A | 61 | 11.397 | −17.618 | 24.255 | 1.00 | 73.54 | A |
| ATOM | 475 | CA | GLY | A | 61 | 12.276 | −18.425 | 23.437 | 1.00 | 76.10 | A |
| ATOM | 476 | C | GLY | A | 61 | 13.151 | −19.318 | 24.282 | 1.00 | 77.80 | A |
| ATOM | 477 | O | GLY | A | 61 | 13.572 | −18.931 | 25.367 | 1.00 | 80.97 | A |
| ATOM | 478 | N | GLN | A | 62 | 13.413 | −20.523 | 23.789 | 1.00 | 113.69 | A |
| ATOM | 479 | CA | GLN | A | 62 | 14.258 | −21.473 | 24.496 | 1.00 | 114.00 | A |
| ATOM | 480 | CB | GLN | A | 62 | 13.862 | −22.907 | 24.136 | 1.00 | 118.81 | A |
| ATOM | 481 | CG | GLN | A | 62 | 12.408 | −23.246 | 24.415 | 1.00 | 122.94 | A |
| ATOM | 482 | CD | GLN | A | 62 | 12.072 | −23.195 | 25.892 | 1.00 | 128.88 | A |
| ATOM | 483 | OE1 | GLN | A | 62 | 12.218 | −22.158 | 26.538 | 1.00 | 128.48 | A |
| ATOM | 484 | NE2 | GLN | A | 62 | 11.619 | −24.319 | 26.435 | 1.00 | 129.05 | A |
| ATOM | 485 | C | GLN | A | 62 | 15.680 | −21.203 | 24.037 | 1.00 | 111.94 | A |
| ATOM | 486 | O | GLN | A | 62 | 15.971 | −21.284 | 22.845 | 1.00 | 113.45 | A |
| ATOM | 487 | N | GLY | A | 63 | 16.560 | −20.870 | 24.975 | 1.00 | 101.90 | A |
| ATOM | 488 | CA | GLY | A | 63 | 17.938 | −20.588 | 24.614 | 1.00 | 99.89 | A |
| ATOM | 489 | C | GLY | A | 63 | 18.019 | −19.526 | 23.531 | 1.00 | 97.97 | A |
| ATOM | 490 | O | GLY | A | 63 | 17.017 | −18.889 | 23.219 | 1.00 | 96.01 | A |
| ATOM | 491 | N | CYS | A | 64 | 19.197 | −19.343 | 22.939 | 1.00 | 115.95 | A |
| ATOM | 492 | CA | CYS | A | 64 | 19.366 | −18.328 | 21.903 | 1.00 | 120.19 | A |
| ATOM | 493 | C | CYS | A | 64 | 19.663 | −18.799 | 20.490 | 1.00 | 126.28 | A |
| ATOM | 494 | O | CYS | A | 64 | 20.406 | −19.754 | 20.278 | 1.00 | 129.19 | A |
| ATOM | 495 | CB | CYS | A | 64 | 20.437 | −17.334 | 22.326 | 1.00 | 130.40 | A |
| ATOM | 496 | SG | CYS | A | 64 | 19.776 | −16.115 | 23.489 | 1.00 | 131.61 | A |
| ATOM | 497 | N | PRO | A | 65 | 19.068 | −18.122 | 19.497 | 1.00 | 125.37 | A |
| ATOM | 498 | CD | PRO | A | 65 | 17.941 | −17.190 | 19.664 | 1.00 | 142.61 | A |
| ATOM | 499 | CA | PRO | A | 65 | 19.253 | −18.448 | 18.082 | 1.00 | 127.71 | A |
| ATOM | 500 | CB | PRO | A | 65 | 18.138 | −17.658 | 17.393 | 1.00 | 145.14 | A |
| ATOM | 501 | CG | PRO | A | 65 | 17.101 | −17.513 | 18.461 | 1.00 | 144.87 | A |
| ATOM | 502 | C | PRO | A | 65 | 20.629 | −18.018 | 17.595 | 1.00 | 130.72 | A |
| ATOM | 503 | O | PRO | A | 65 | 21.585 | −17.946 | 18.367 | 1.00 | 130.99 | A |
| ATOM | 504 | N | SER | A | 66 | 20.711 | −17.722 | 16.305 | 1.00 | 178.92 | A |
| ATOM | 505 | CA | SER | A | 66 | 21.955 | −17.295 | 15.690 | 1.00 | 182.52 | A |
| ATOM | 506 | CB | SER | A | 66 | 22.077 | −17.907 | 14.293 | 1.00 | 160.62 | A |
| ATOM | 507 | OG | SER | A | 66 | 23.265 | −17.484 | 13.651 | 1.00 | 157.78 | A |
| ATOM | 508 | C | SER | A | 66 | 21.998 | −15.775 | 15.595 | 1.00 | 183.07 | A |
| ATOM | 509 | O | SER | A | 66 | 21.037 | −15.093 | 15.953 | 1.00 | 176.49 | A |
| ATOM | 510 | N | THR | A | 67 | 23.126 | −15.263 | 15.111 | 1.00 | 200.72 | A |
| ATOM | 511 | CA | THR | A | 67 | 23.355 | −13.830 | 14.938 | 1.00 | 200.72 | A |
| ATOM | 512 | CB | THR | A | 67 | 23.038 | −13.385 | 13.488 | 1.00 | 168.99 | A |
| ATOM | 513 | OG1 | THR | A | 67 | 23.758 | −14.211 | 12.564 | 1.00 | 168.99 | A |
| ATOM | 514 | CG2 | THR | A | 67 | 23.444 | −11.928 | 13.274 | 1.00 | 168.99 | A |
| ATOM | 515 | C | THR | A | 67 | 22.558 | −12.948 | 15.892 | 1.00 | 200.72 | A |
| ATOM | 516 | O | THR | A | 67 | 22.943 | −12.753 | 17.045 | 1.00 | 200.72 | A |
| ATOM | 517 | N | HIS | A | 68 | 21.441 | −12.422 | 15.402 | 1.00 | 139.45 | A |
| ATOM | 518 | CA | HIS | A | 68 | 20.605 | −11.543 | 16.201 | 1.00 | 134.57 | A |
| ATOM | 519 | CB | HIS | A | 68 | 21.077 | −10.099 | 16.016 | 1.00 | 185.55 | A |
| ATOM | 520 | CG | HIS | A | 68 | 20.682 | −9.181 | 17.129 | 1.00 | 185.55 | A |
| ATOM | 521 | CD2 | HIS | A | 68 | 19.930 | −8.055 | 17.133 | 1.00 | 185.55 | A |
| ATOM | 522 | ND1 | HIS | A | 68 | 21.095 | −9.370 | 18.430 | 1.00 | 185.55 | A |
| ATOM | 523 | CE1 | HIS | A | 68 | 20.615 | −8.400 | 19.188 | 1.00 | 185.55 | A |
| ATOM | 524 | NE2 | HIS | A | 68 | 19.904 | −7.589 | 18.425 | 1.00 | 185.55 | A |
| ATOM | 525 | C | HIS | A | 68 | 19.141 | −11.675 | 15.782 | 1.00 | 130.40 | A |
| ATOM | 526 | O | HIS | A | 68 | 18.837 | −11.840 | 14.599 | 1.00 | 129.85 | A |
| ATOM | 527 | N | VAL | A | 69 | 18.241 | −11.607 | 16.760 | 1.00 | 112.98 | A |
| ATOM | 528 | CA | VAL | A | 69 | 16.808 | −11.710 | 16.500 | 1.00 | 112.71 | A |
| ATOM | 529 | CB | VAL | A | 69 | 16.185 | −12.924 | 17.226 | 1.00 | 126.62 | A |
| ATOM | 530 | CG1 | VAL | A | 69 | 14.709 | −13.022 | 16.895 | 1.00 | 126.38 | A |
| ATOM | 531 | CG2 | VAL | A | 69 | 16.901 | −14.203 | 16.820 | 1.00 | 124.88 | A |
| ATOM | 532 | C | VAL | A | 69 | 16.100 | −10.450 | 16.981 | 1.00 | 112.30 | A |
| ATOM | 533 | O | VAL | A | 69 | 16.349 | −9.970 | 18.087 | 1.00 | 115.86 | A |
| ATOM | 534 | N | LEU | A | 70 | 15.222 | −9.914 | 16.141 | 1.00 | 76.21 | A |
| ATOM | 535 | CA | LEU | A | 70 | 14.473 | −8.717 | 16.491 | 1.00 | 71.43 | A |
| ATOM | 536 | CB | LEU | A | 70 | 14.644 | −7.633 | 15.426 | 1.00 | 89.36 | A |
| ATOM | 537 | CG | LEU | A | 70 | 16.019 | −6.977 | 15.290 | 1.00 | 91.06 | A |
| ATOM | 538 | CD1 | LEU | A | 70 | 15.935 | −5.868 | 14.258 | 1.00 | 86.10 | A |
| ATOM | 539 | CD2 | LEU | A | 70 | 16.473 | −6.418 | 16.627 | 1.00 | 91.66 | A |
| ATOM | 540 | C | LEU | A | 70 | 12.995 | −9.028 | 16.641 | 1.00 | 72.47 | A |
| ATOM | 541 | O | LEU | A | 70 | 12.411 | −9.728 | 15.818 | 1.00 | 72.58 | A |
| ATOM | 542 | N | LEU | A | 71 | 12.400 | −8.503 | 17.704 | 1.00 | 70.90 | A |
| ATOM | 543 | CA | LEU | A | 71 | 10.986 | −8.701 | 17.972 | 1.00 | 70.84 | A |
| ATOM | 544 | CB | LEU | A | 71 | 10.791 | −9.209 | 19.401 | 1.00 | 62.08 | A |
| ATOM | 545 | CG | LEU | A | 71 | 11.477 | −10.532 | 19.745 | 1.00 | 59.75 | A |
| ATOM | 546 | CD1 | LEU | A | 71 | 11.270 | −10.856 | 21.221 | 1.00 | 55.90 | A |
| ATOM | 547 | CD2 | LEU | A | 71 | 10.910 | −11.633 | 18.866 | 1.00 | 59.87 | A |
| ATOM | 548 | C | LEU | A | 71 | 10.283 | −7.363 | 17.803 | 1.00 | 74.87 | A |
| ATOM | 549 | O | LEU | A | 71 | 10.690 | −6.365 | 18.402 | 1.00 | 74.36 | A |
| ATOM | 550 | N | THR | A | 72 | 9.246 | −7.336 | 16.973 | 1.00 | 66.05 | A |
| ATOM | 551 | CA | THR | A | 72 | 8.500 | −6.103 | 16.759 | 1.00 | 63.48 | A |
| ATOM | 552 | CB | THR | A | 72 | 8.682 | −5.539 | 15.339 | 1.00 | 65.28 | A |
| ATOM | 553 | OG1 | THR | A | 72 | 7.885 | −6.292 | 14.415 | 1.00 | 67.81 | A |
| ATOM | 554 | CG2 | THR | A | 72 | 10.136 | −5.603 | 14.932 | 1.00 | 63.00 | A |
| ATOM | 555 | C | THR | A | 72 | 7.008 | −6.303 | 16.981 | 1.00 | 66.11 | A |
| ATOM | 556 | O | THR | A | 72 | 6.437 | −7.343 | 16.628 | 1.00 | 67.15 | A |
| ATOM | 557 | N | HIS | A | 73 | 6.388 | −5.289 | 17.574 | 1.00 | 47.67 | A |
| ATOM | 558 | CA | HIS | A | 73 | 4.964 | −5.307 | 17.853 | 1.00 | 48.12 | A |
| ATOM | 559 | CB | HIS | A | 73 | 4.719 | −5.389 | 19.357 | 1.00 | 57.38 | A |
| ATOM | 560 | CG | HIS | A | 73 | 3.277 | −5.513 | 19.719 | 1.00 | 60.25 | A |
| ATOM | 561 | CD2 | HIS | A | 73 | 2.207 | −5.898 | 18.987 | 1.00 | 62.01 | A |
| ATOM | 562 | ND1 | HIS | A | 73 | 2.800 | −5.235 | 20.982 | 1.00 | 64.86 | A |
| ATOM | 563 | CE1 | HIS | A | 73 | 1.496 | −5.442 | 21.013 | 1.00 | 63.40 | A |
| ATOM | 564 | NE2 | HIS | A | 73 | 1.112 | −5.846 | 19.815 | 1.00 | 63.57 | A |
| ATOM | 565 | C | HIS | A | 73 | 4.487 | −3.976 | 17.327 | 1.00 | 47.20 | A |
| ATOM | 566 | O | HIS | A | 73 | 5.119 | −2.952 | 17.571 | 1.00 | 43.06 | A |
| ATOM | 567 | N | THR | A | 74 | 3.390 | −3.971 | 16.591 | 1.00 | 36.40 | A |
| ATOM | 568 | CA | THR | A | 74 | 2.919 | −2.718 | 16.045 | 1.00 | 42.99 | A |
| ATOM | 569 | CB | THR | A | 74 | 3.764 | −2.332 | 14.774 | 1.00 | 50.66 | A |
| ATOM | 570 | OG1 | THR | A | 74 | 2.899 | −1.913 | 13.713 | 1.00 | 53.17 | A |
| ATOM | 571 | CG2 | THR | A | 74 | 4.618 | −3.503 | 14.306 | 1.00 | 55.49 | A |
| ATOM | 572 | C | THR | A | 74 | 1.424 | −2.706 | 15.740 | 1.00 | 41.84 | A |
| ATOM | 573 | O | THR | A | 74 | 0.864 | −3.699 | 15.286 | 1.00 | 37.42 | A |
| ATOM | 574 | N | ILE | A | 75 | 0.776 | −1.583 | 16.029 | 1.00 | 53.82 | A |
| ATOM | 575 | CA | ILE | A | 75 | −0.647 | −1.435 | 15.765 | 1.00 | 55.12 | A |
| ATOM | 576 | CB | ILE | A | 75 | −1.396 | −0.839 | 16.974 | 1.00 | 34.45 | A |
| ATOM | 577 | CG2 | ILE | A | 75 | −2.852 | −0.579 | 16.619 | 1.00 | 33.94 | A |
| ATOM | 578 | CG1 | ILE | A | 75 | −1.302 | −1.804 | 18.158 | 1.00 | 31.61 | A |
| ATOM | 579 | CD1 | ILE | A | 75 | −2.023 | −1.325 | 19.390 | 1.00 | 34.90 | A |
| ATOM | 580 | C | ILE | A | 75 | −0.787 | −0.491 | 14.583 | 1.00 | 58.26 | A |
| ATOM | 581 | O | ILE | A | 75 | −0.305 | 0.647 | 14.621 | 1.00 | 55.03 | A |
| ATOM | 582 | N | SER | A | 76 | −1.443 | −0.970 | 13.532 | 1.00 | 56.70 | A |
| ATOM | 583 | CA | SER | A | 76 | −1.631 | −0.184 | 12.327 | 1.00 | 57.78 | A |
| ATOM | 584 | CB | SER | A | 76 | −1.209 | −0.983 | 11.098 | 1.00 | 52.29 | A |
| ATOM | 585 | OG | SER | A | 76 | 0.133 | −1.405 | 11.208 | 1.00 | 61.58 | A |
| ATOM | 586 | C | SER | A | 76 | −3.064 | 0.233 | 12.125 | 1.00 | 53.78 | A |
| ATOM | 587 | O | SER | A | 76 | −3.995 | −0.405 | 12.621 | 1.00 | 49.93 | A |
| ATOM | 588 | N | ARG | A | 77 | −3.231 | 1.321 | 11.384 | 1.00 | 43.34 | A |
| ATOM | 589 | CA | ARG | A | 77 | −4.549 | 1.806 | 11.039 | 1.00 | 48.52 | A |
| ATOM | 590 | CB | ARG | A | 77 | −4.784 | 3.231 | 11.542 | 1.00 | 39.05 | A |
| ATOM | 591 | CG | ARG | A | 77 | −5.906 | 3.930 | 10.771 | 1.00 | 41.23 | A |
| ATOM | 592 | CD | ARG | A | 77 | −6.560 | 5.051 | 11.549 | 1.00 | 42.10 | A |
| ATOM | 593 | NE | ARG | A | 77 | −6.212 | 6.386 | 11.068 | 1.00 | 44.54 | A |
| ATOM | 594 | CZ | ARG | A | 77 | −7.106 | 7.307 | 10.718 | 1.00 | 47.97 | A |
| ATOM | 595 | NH1 | ARG | A | 77 | −6.708 | 8.496 | 10.298 | 1.00 | 48.06 | A |
| ATOM | 596 | NH2 | ARG | A | 77 | −8.402 | 7.036 | 10.770 | 1.00 | 47.83 | A |
| ATOM | 597 | C | ARG | A | 77 | −4.635 | 1.814 | 9.523 | 1.00 | 46.86 | A |
| ATOM | 598 | O | ARG | A | 77 | −3.716 | 2.262 | 8.852 | 1.00 | 52.61 | A |
| ATOM | 599 | N | ILE | A | 78 | −5.716 | 1.272 | 8.983 | 1.00 | 47.21 | A |
| ATOM | 600 | CA | ILE | A | 78 | −5.926 | 1.320 | 7.548 | 1.00 | 45.90 | A |
| ATOM | 601 | CB | ILE | A | 78 | −5.978 | −0.110 | 6.879 | 1.00 | 56.08 | A |
| ATOM | 602 | CG2 | ILE | A | 78 | −5.464 | −1.168 | 7.825 | 1.00 | 56.58 | A |
| ATOM | 603 | CG1 | ILE | A | 78 | −7.378 | −0.444 | 6.405 | 1.00 | 57.75 | A |
| ATOM | 604 | CD1 | ILE | A | 78 | −7.790 | 0.325 | 5.179 | 1.00 | 58.07 | A |
| ATOM | 605 | C | ILE | A | 78 | −7.252 | 2.098 | 7.509 | 1.00 | 48.07 | A |
| ATOM | 606 | O | ILE | A | 78 | −8.329 | 1.568 | 7.797 | 1.00 | 46.62 | A |
| ATOM | 607 | N | ALA | A | 79 | −7.136 | 3.393 | 7.215 | 1.00 | 51.86 | A |
| ATOM | 608 | CA | ALA | A | 79 | −8.284 | 4.295 | 7.193 | 1.00 | 48.46 | A |
| ATOM | 609 | CB | ALA | A | 79 | −7.793 | 5.735 | 7.248 | 1.00 | 57.50 | A |
| ATOM | 610 | C | ALA | A | 79 | −9.229 | 4.111 | 6.015 | 1.00 | 49.66 | A |
| ATOM | 611 | O | ALA | A | 79 | −8.818 | 3.711 | 4.936 | 1.00 | 49.86 | A |
| ATOM | 612 | N | VAL | A | 80 | −10.503 | 4.408 | 6.229 | 1.00 | 58.04 | A |
| ATOM | 613 | CA | VAL | A | 80 | −11.489 | 4.278 | 5.168 | 1.00 | 60.53 | A |
| ATOM | 614 | CB | VAL | A | 80 | −12.911 | 4.599 | 5.674 | 1.00 | 70.47 | A |
| ATOM | 615 | CG1 | VAL | A | 80 | −13.905 | 4.490 | 4.536 | 1.00 | 68.87 | A |
| ATOM | 616 | CG2 | VAL | A | 80 | −13.295 | 3.656 | 6.781 | 1.00 | 72.17 | A |
| ATOM | 617 | C | VAL | A | 80 | −11.146 | 5.258 | 4.052 | 1.00 | 65.08 | A |
| ATOM | 618 | O | VAL | A | 80 | −11.381 | 4.987 | 2.876 | 1.00 | 63.67 | A |
| ATOM | 619 | N | SER | A | 81 | −10.583 | 6.402 | 4.420 | 1.00 | 62.10 | A |
| ATOM | 620 | CA | SER | A | 81 | −10.227 | 7.404 | 3.428 | 1.00 | 64.96 | A |
| ATOM | 621 | CB | SER | A | 81 | −10.448 | 8.797 | 3.993 | 1.00 | 67.37 | A |
| ATOM | 622 | OG | SER | A | 81 | −11.821 | 8.994 | 4.259 | 1.00 | 70.41 | A |
| ATOM | 623 | C | SER | A | 81 | −8.803 | 7.295 | 2.901 | 1.00 | 69.14 | A |
| ATOM | 624 | O | SER | A | 81 | −8.354 | 8.177 | 2.175 | 1.00 | 69.66 | A |
| ATOM | 625 | N | TYR | A | 82 | −8.099 | 6.220 | 3.257 | 1.00 | 51.69 | A |
| ATOM | 626 | CA | TYR | A | 82 | −6.721 | 6.006 | 2.800 | 1.00 | 53.73 | A |
| ATOM | 627 | CB | TYR | A | 82 | −5.771 | 6.989 | 3.500 | 1.00 | 41.69 | A |
| ATOM | 628 | CG | TYR | A | 82 | −4.372 | 7.001 | 2.925 | 1.00 | 44.89 | A |
| ATOM | 629 | CD1 | TYR | A | 82 | −3.260 | 6.692 | 3.721 | 1.00 | 47.18 | A |
| ATOM | 630 | CE1 | TYR | A | 82 | −1.980 | 6.641 | 3.175 | 1.00 | 48.97 | A |
| ATOM | 631 | CD2 | TYR | A | 82 | −4.161 | 7.270 | 1.567 | 1.00 | 46.98 | A |
| ATOM | 632 | CE2 | TYR | A | 82 | −2.887 | 7.226 | 1.013 | 1.00 | 48.45 | A |
| ATOM | 633 | CZ | TYR | A | 82 | −1.803 | 6.908 | 1.816 | 1.00 | 49.14 | A |
| ATOM | 634 | OH | TYR | A | 82 | −0.549 | 6.843 | 1.259 | 1.00 | 47.56 | A |
| ATOM | 635 | C | TYR | A | 82 | −6.294 | 4.565 | 3.087 | 1.00 | 53.16 | A |
| ATOM | 636 | O | TYR | A | 82 | −5.499 | 4.312 | 3.989 | 1.00 | 53.34 | A |
| ATOM | 637 | N | GLN | A | 83 | −6.834 | 3.632 | 2.303 | 1.00 | 61.97 | A |
| ATOM | 638 | CA | GLN | A | 83 | −6.565 | 2.202 | 2.449 | 1.00 | 61.86 | A |
| ATOM | 639 | CB | GLN | A | 83 | −7.279 | 1.407 | 1.351 | 1.00 | 86.61 | A |
| ATOM | 640 | CG | GLN | A | 83 | −8.611 | 0.787 | 1.741 | 1.00 | 90.88 | A |
| ATOM | 641 | CD | GLN | A | 83 | −9.738 | 1.794 | 1.783 | 1.00 | 91.92 | A |
| ATOM | 642 | OE1 | GLN | A | 83 | −10.909 | 1.423 | 1.837 | 1.00 | 90.94 | A |
| ATOM | 643 | NE2 | GLN | A | 83 | −9.392 | 3.078 | 1.761 | 1.00 | 89.42 | A |
| ATOM | 644 | C | GLN | A | 83 | −5.108 | 1.771 | 2.456 | 1.00 | 60.04 | A |
| ATOM | 645 | O | GLN | A | 83 | −4.646 | 1.171 | 1.500 | 1.00 | 57.49 | A |
| ATOM | 646 | N | THR | A | 84 | −4.381 | 2.050 | 3.526 | 1.00 | 64.19 | A |
| ATOM | 647 | CA | THR | A | 84 | −2.993 | 1.626 | 3.605 | 1.00 | 56.56 | A |
| ATOM | 648 | CB | THR | A | 84 | −2.045 | 2.607 | 2.933 | 1.00 | 89.07 | A |
| ATOM | 649 | OG1 | THR | A | 84 | −2.526 | 2.908 | 1.623 | 1.00 | 89.62 | A |
| ATOM | 650 | CG2 | THR | A | 84 | −0.646 | 2.001 | 2.836 | 1.00 | 91.82 | A |
| ATOM | 651 | C | THR | A | 84 | −2.584 | 1.543 | 5.051 | 1.00 | 53.07 | A |
| ATOM | 652 | O | THR | A | 84 | −2.915 | 2.422 | 5.845 | 1.00 | 54.38 | A |
| ATOM | 653 | N | LYS | A | 85 | −1.861 | 0.491 | 5.401 | 1.00 | 50.48 | A |
| ATOM | 654 | CA | LYS | A | 85 | −1.408 | 0.352 | 6.760 | 1.00 | 50.34 | A |
| ATOM | 655 | CB | LYS | A | 85 | −0.647 | −0.957 | 6.941 | 1.00 | 74.05 | A |
| ATOM | 656 | CG | LYS | A | 85 | −1.473 | −2.140 | 7.405 | 1.00 | 70.99 | A |
| ATOM | 657 | CD | LYS | A | 85 | −0.538 | −3.307 | 7.740 | 1.00 | 69.39 | A |
| ATOM | 658 | CE | LYS | A | 85 | −1.272 | −4.483 | 8.374 | 1.00 | 67.69 | A |
| ATOM | 659 | NZ | LYS | A | 85 | −2.299 | −5.057 | 7.458 | 1.00 | 65.94 | A |
| ATOM | 660 | C | LYS | A | 85 | −0.470 | 1.507 | 7.095 | 1.00 | 50.14 | A |
| ATOM | 661 | O | LYS | A | 85 | 0.512 | 1.734 | 6.386 | 1.00 | 50.59 | A |
| ATOM | 662 | N | VAL | A | 86 | −0.795 | 2.259 | 8.143 | 1.00 | 51.20 | A |
| ATOM | 663 | CA | VAL | A | 86 | 0.073 | 3.331 | 8.616 | 1.00 | 54.09 | A |
| ATOM | 664 | CB | VAL | A | 86 | −0.530 | 4.754 | 8.411 | 1.00 | 66.12 | A |
| ATOM | 665 | CG1 | VAL | A | 86 | −0.710 | 5.026 | 6.940 | 1.00 | 72.86 | A |
| ATOM | 666 | CG2 | VAL | A | 86 | −1.850 | 4.890 | 9.116 | 1.00 | 67.39 | A |
| ATOM | 667 | C | VAL | A | 86 | 0.225 | 3.012 | 10.098 | 1.00 | 54.76 | A |
| ATOM | 668 | O | VAL | A | 86 | −0.762 | 2.771 | 10.788 | 1.00 | 59.45 | A |
| ATOM | 669 | N | ASN | A | 87 | 1.457 | 2.965 | 10.584 | 1.00 | 67.49 | A |
| ATOM | 670 | CA | ASN | A | 87 | 1.695 | 2.639 | 11.986 | 1.00 | 64.17 | A |
| ATOM | 671 | CB | ASN | A | 87 | 3.172 | 2.316 | 12.193 | 1.00 | 58.77 | A |
| ATOM | 672 | CG | ASN | A | 87 | 3.599 | 1.044 | 11.476 | 1.00 | 59.07 | A |
| ATOM | 673 | OD1 | ASN | A | 87 | 2.771 | 0.295 | 10.941 | 1.00 | 61.68 | A |
| ATOM | 674 | ND2 | ASN | A | 87 | 4.900 | 0.785 | 11.476 | 1.00 | 56.03 | A |
| ATOM | 675 | C | ASN | A | 87 | 1.272 | 3.712 | 12.992 | 1.00 | 62.99 | A |
| ATOM | 676 | O | ASN | A | 87 | 1.615 | 4.889 | 12.845 | 1.00 | 61.57 | A |
| ATOM | 677 | N | LEU | A | 88 | 0.529 | 3.311 | 14.023 | 1.00 | 50.94 | A |
| ATOM | 678 | CA | LEU | A | 88 | 0.105 | 4.252 | 15.061 | 1.00 | 49.93 | A |
| ATOM | 679 | CB | LEU | A | 88 | −1.319 | 3.981 | 15.499 | 1.00 | 35.26 | A |
| ATOM | 680 | CG | LEU | A | 88 | −2.386 | 4.084 | 14.429 | 1.00 | 32.19 | A |
| ATOM | 681 | CD1 | LEU | A | 88 | −3.741 | 3.814 | 15.070 | 1.00 | 32.96 | A |
| ATOM | 682 | CD2 | LEU | A | 88 | −2.332 | 5.460 | 13.779 | 1.00 | 30.76 | A |
| ATOM | 683 | C | LEU | A | 88 | 1.002 | 4.096 | 16.270 | 1.00 | 49.17 | A |
| ATOM | 684 | O | LEU | A | 88 | 1.427 | 5.064 | 16.870 | 1.00 | 47.64 | A |
| ATOM | 685 | N | LEU | A | 89 | 1.283 | 2.852 | 16.622 | 1.00 | 39.72 | A |
| ATOM | 686 | CA | LEU | A | 89 | 2.122 | 2.538 | 17.766 | 1.00 | 38.64 | A |
| ATOM | 687 | CB | LEU | A | 89 | 1.247 | 2.118 | 18.940 | 1.00 | 36.86 | A |
| ATOM | 688 | CG | LEU | A | 89 | 0.117 | 3.066 | 19.328 | 1.00 | 40.84 | A |
| ATOM | 689 | CDL | LEU | A | 89 | −0.916 | 2.303 | 20.130 | 1.00 | 40.57 | A |
| ATOM | 690 | CD2 | LEU | A | 89 | 0.664 | 4.223 | 20.119 | 1.00 | 39.96 | A |
| ATOM | 691 | C | LEU | A | 89 | 3.018 | 1.376 | 17.375 | 1.00 | 40.31 | A |
| ATOM | 692 | O | LEU | A | 89 | 2.576 | 0.433 | 16.711 | 1.00 | 41.03 | A |
| ATOM | 693 | N | SER | A | 90 | 4.272 | 1.434 | 17.798 | 1.00 | 47.00 | A |
| ATOM | 694 | CA | SER | A | 90 | 5.225 | 0.384 | 17.464 | 1.00 | 46.34 | A |
| ATOM | 695 | CB | SER | A | 90 | 5.712 | 0.566 | 16.025 | 1.00 | 79.05 | A |
| ATOM | 696 | OG | SER | A | 90 | 6.704 | −0.392 | 15.702 | 1.00 | 83.27 | A |
| ATOM | 697 | C | SER | A | 90 | 6.430 | 0.351 | 18.385 | 1.00 | 44.97 | A |
| ATOM | 698 | O | SER | A | 90 | 6.908 | 1.387 | 18.838 | 1.00 | 44.79 | A |
| ATOM | 699 | N | ALA | A | 91 | 6.926 | −0.845 | 18.658 | 1.00 | 44.34 | A |
| ATOM | 700 | CA | ALA | A | 91 | 8.095 | −0.985 | 19.508 | 1.00 | 42.43 | A |
| ATOM | 701 | CB | ALA | A | 91 | 7.674 | −1.180 | 20.955 | 1.00 | 30.60 | A |
| ATOM | 702 | C | ALA | A | 91 | 8.938 | −2.162 | 19.023 | 1.00 | 46.40 | A |
| ATOM | 703 | O | ALA | A | 91 | 8.445 | −3.048 | 18.323 | 1.00 | 48.27 | A |
| ATOM | 704 | N | ILE | A | 92 | 10.214 | −2.154 | 19.392 | 1.00 | 45.16 | A |
| ATOM | 705 | CA | ILE | A | 92 | 11.144 | −3.196 | 18.990 | 1.00 | 45.14 | A |
| ATOM | 706 | CB | ILE | A | 92 | 12.160 | −2.651 | 17.974 | 1.00 | 55.56 | A |
| ATOM | 707 | CG2 | ILE | A | 92 | 13.040 | −3.768 | 17.478 | 1.00 | 61.63 | A |
| ATOM | 708 | CG1 | ILE | A | 92 | 11.423 | −2.010 | 16.796 | 1.00 | 53.67 | A |
| ATOM | 709 | CD1 | ILE | A | 92 | 12.329 | −1.290 | 15.821 | 1.00 | 59.54 | A |
| ATOM | 710 | C | ILE | A | 92 | 11.893 | −3.657 | 20.221 | 1.00 | 44.97 | A |
| ATOM | 711 | O | ILE | A | 92 | 12.143 | −2.861 | 21.123 | 1.00 | 46.05 | A |
| ATOM | 712 | N | LYS | A | 93 | 12.252 | −4.936 | 20.262 | 1.00 | 67.76 | A |
| ATOM | 713 | CA | LYS | A | 93 | 12.985 | −5.474 | 21.403 | 1.00 | 68.41 | A |
| ATOM | 714 | CB | LYS | A | 93 | 12.008 | −6.066 | 22.418 | 1.00 | 57.46 | A |
| ATOM | 715 | CG | LYS | A | 93 | 12.475 | −5.970 | 23.853 | 1.00 | 61.15 | A |
| ATOM | 716 | CD | LYS | A | 93 | 11.288 | −5.984 | 24.821 | 1.00 | 60.92 | A |
| ATOM | 717 | CE | LYS | A | 93 | 11.726 | −5.743 | 26.281 | 1.00 | 60.82 | A |
| ATOM | 718 | NZ | LYS | A | 93 | 10.595 | −5.710 | 27.271 | 1.00 | 63.39 | A |
| ATOM | 719 | C | LYS | A | 93 | 13.967 | −6.537 | 20.926 | 1.00 | 70.11 | A |
| ATOM | 720 | O | LYS | A | 93 | 13.622 | −7.376 | 20.092 | 1.00 | 68.03 | A |
| ATOM | 721 | N | SER | A | 94 | 15.192 | −6.485 | 21.452 | 1.00 | 69.23 | A |
| ATOM | 722 | CA | SER | A | 94 | 16.257 | −7.430 | 21.092 | 1.00 | 71.68 | A |
| ATOM | 723 | CB | SER | A | 94 | 17.544 | −6.669 | 20.770 | 1.00 | 87.28 | A |
| ATOM | 724 | OG | SER | A | 94 | 17.959 | −5.877 | 21.876 | 1.00 | 89.71 | A |
| ATOM | 725 | C | SER | A | 94 | 16.522 | −8.403 | 22.238 | 1.00 | 74.27 | A |
| ATOM | 726 | O | SER | A | 94 | 17.280 | −8.101 | 23.159 | 1.00 | 75.54 | A |
| ATOM | 727 | N | PRO | A | 95 | 15.916 | −9.597 | 22.181 | 1.00 | 74.98 | A |
| ATOM | 728 | CD | PRO | A | 95 | 15.186 | −10.141 | 21.024 | 1.00 | 66.26 | A |
| ATOM | 729 | CA | PRO | A | 95 | 16.075 | −10.620 | 23.218 | 1.00 | 78.92 | A |
| ATOM | 730 | CB | PRO | A | 95 | 15.105 | −11.705 | 22.774 | 1.00 | 69.20 | A |
| ATOM | 731 | CG | PRO | A | 95 | 15.209 | −11.630 | 21.292 | 1.00 | 66.65 | A |
| ATOM | 732 | C | PRO | A | 95 | 17.495 | −11.128 | 23.333 | 1.00 | 83.35 | A |
| ATOM | 733 | O | PRO | A | 95 | 18.224 | −10.765 | 24.255 | 1.00 | 86.78 | A |
| ATOM | 734 | N | CYS | A | 96 | 17.882 | −11.976 | 22.390 | 1.00 | 168.79 | A |
| ATOM | 735 | CA | CYS | A | 96 | 19.218 | −12.534 | 22.380 | 1.00 | 168.58 | A |
| ATOM | 736 | C | CYS | A | 96 | 20.195 | −11.536 | 21.794 | 1.00 | 169.68 | A |
| ATOM | 737 | O | CYS | A | 96 | 20.196 | −11.279 | 20.589 | 1.00 | 169.98 | A |
| ATOM | 738 | CB | CYS | A | 96 | 19.244 | −13.828 | 21.573 | 1.00 | 200.84 | A |
| ATOM | 739 | SG | CYS | A | 96 | 18.348 | −15.190 | 22.378 | 1.00 | 200.84 | A |
| ATOM | 740 | N | GLN | A | 97 | 21.019 | −10.969 | 22.667 | 1.00 | 200.84 | A |
| ATOM | 741 | CA | GLN | A | 97 | 22.024 | −9.996 | 22.269 | 1.00 | 200.84 | A |
| ATOM | 742 | CB | GLN | A | 97 | 22.488 | −9.210 | 23.496 | 1.00 | 148.53 | A |
| ATOM | 743 | CG | GLN | A | 97 | 21.349 | −8.598 | 24.295 | 1.00 | 148.53 | A |
| ATOM | 744 | CD | GLN | A | 97 | 21.773 | −8.193 | 25.692 | 1.00 | 148.53 | A |
| ATOM | 745 | OE1 | GLN | A | 97 | 22.682 | −7.380 | 25.868 | 1.00 | 148.53 | A |
| ATOM | 746 | NE2 | GLN | A | 97 | 21.115 | −8.761 | 26.697 | 1.00 | 148.53 | A |
| ATOM | 747 | C | GLN | A | 97 | 23.203 | −10.744 | 21.647 | 1.00 | 200.84 | A |
| ATOM | 748 | O | GLN | A | 97 | 24.361 | −10.449 | 21.943 | 1.00 | 200.84 | A |
| ATOM | 749 | N | ARG | A | 98 | 22.889 | −11.719 | 20.792 | 1.00 | 200.84 | A |
| ATOM | 750 | CA | ARG | A | 98 | 23.892 | −12.537 | 20.111 | 1.00 | 200.84 | A |
| ATOM | 751 | CB | ARG | A | 98 | 24.955 | −11.633 | 19.464 | 1.00 | 165.85 | A |
| ATOM | 752 | CG | ARG | A | 98 | 26.154 | −12.360 | 18.862 | 1.00 | 165.85 | A |
| ATOM | 753 | CD | ARG | A | 98 | 25.766 | −13.268 | 17.706 | 1.00 | 165.85 | A |
| ATOM | 754 | NE | ARG | A | 98 | 26.881 | −14.114 | 17.290 | 1.00 | 165.85 | A |
| ATOM | 755 | CZ | ARG | A | 98 | 28.071 | −13.657 | 16.910 | 1.00 | 165.85 | A |
| ATOM | 756 | NH1 | ARG | A | 98 | 29.023 | −14.508 | 16.550 | 1.00 | 165.85 | A |
| ATOM | 757 | NH2 | ARG | A | 98 | 28.312 | −12.352 | 16.887 | 1.00 | 165.85 | A |
| ATOM | 758 | C | ARG | A | 98 | 24.551 | −13.557 | 21.051 | 1.00 | 200.84 | A |
| ATOM | 759 | O | ARG | A | 98 | 25.436 | −14.310 | 20.639 | 1.00 | 200.84 | A |
| ATOM | 760 | N | GLU | A | 99 | 24.103 | −13.591 | 22.306 | 1.00 | 200.68 | A |
| ATOM | 761 | CA | GLU | A | 99 | 24.649 | −14.516 | 23.301 | 1.00 | 200.68 | A |
| ATOM | 762 | CB | GLU | A | 99 | 23.711 | −14.628 | 24.511 | 1.00 | 183.53 | A |
| ATOM | 763 | CG | GLU | A | 99 | 22.369 | −13.928 | 24.346 | 1.00 | 183.53 | A |
| ATOM | 764 | CD | GLU | A | 99 | 22.425 | −12.458 | 24.715 | 1.00 | 183.53 | A |
| ATOM | 765 | OE1 | GLU | A | 99 | 23.396 | −11.783 | 24.315 | 1.00 | 183.53 | A |
| ATOM | 766 | OE2 | GLU | A | 99 | 21.494 | −11.977 | 25.398 | 1.00 | 183.53 | A |
| ATOM | 767 | C | GLU | A | 99 | 24.891 | −15.910 | 22.726 | 1.00 | 200.68 | A |
| ATOM | 768 | O | GLU | A | 99 | 25.975 | −16.468 | 22.884 | 1.00 | 200.68 | A |
| ATOM | 769 | N | THR | A | 100 | 23.876 | −16.459 | 22.061 | 1.00 | 177.94 | A |
| ATOM | 770 | CA | THR | A | 100 | 23.949 | −17.788 | 21.456 | 1.00 | 177.94 | A |
| ATOM | 771 | CB | THR | A | 100 | 24.786 | −17.772 | 20.154 | 1.00 | 163.57 | A |
| ATOM | 772 | OG1 | THR | A | 100 | 26.090 | −17.247 | 20.426 | 1.00 | 163.85 | A |
| ATOM | 773 | CG2 | THR | A | 100 | 24.113 | −16.913 | 19.095 | 1.00 | 164.74 | A |
| ATOM | 774 | C | THR | A | 100 | 24.528 | −18.837 | 22.408 | 1.00 | 177.94 | A |
| ATOM | 775 | O | THR | A | 100 | 25.691 | −18.762 | 22.805 | 1.00 | 177.94 | A |
| ATOM | 776 | N | PRO | A | 101 | 23.715 | −19.837 | 22.783 | 1.00 | 200.53 | A |
| ATOM | 777 | CD | PRO | A | 101 | 22.340 | −20.070 | 22.305 | 1.00 | 113.70 | A |
| ATOM | 778 | CA | PRO | A | 101 | 24.132 | −20.909 | 23.691 | 1.00 | 200.53 | A |
| ATOM | 779 | CB | PRO | A | 101 | 23.046 | −21.963 | 23.491 | 1.00 | 114.65 | A |
| ATOM | 780 | CG | PRO | A | 101 | 21.831 | −21.125 | 23.271 | 1.00 | 112.68 | A |
| ATOM | 781 | C | PRO | A | 101 | 25.534 | −21.446 | 23.401 | 1.00 | 200.53 | A |
| ATOM | 782 | O | PRO | A | 101 | 25.768 | −22.088 | 22.376 | 1.00 | 200.53 | A |
| ATOM | 783 | N | GLU | A | 102 | 26.457 | −21.172 | 24.317 | 1.00 | 191.30 | A |
| ATOM | 784 | CA | GLU | A | 102 | 27.842 | −21.612 | 24.198 | 1.00 | 191.30 | A |
| ATOM | 785 | CB | GLU | A | 102 | 28.561 | −21.394 | 25.538 | 1.00 | 147.13 | A |
| ATOM | 786 | CG | GLU | A | 102 | 30.004 | −21.885 | 25.600 | 1.00 | 148.33 | A |
| ATOM | 787 | CD | GLU | A | 102 | 30.114 | −23.399 | 25.683 | 1.00 | 149.64 | A |
| ATOM | 788 | OE1 | GLU | A | 102 | 29.577 | −23.990 | 26.648 | 1.00 | 150.45 | A |
| ATOM | 789 | OE2 | GLU | A | 102 | 30.740 | −23.997 | 24.782 | 1.00 | 148.76 | A |
| ATOM | 790 | C | GLU | A | 102 | 27.940 | −23.079 | 23.770 | 1.00 | 187.86 | A |
| ATOM | 791 | O | GLU | A | 102 | 28.800 | −23.441 | 22.965 | 1.00 | 184.92 | A |
| ATOM | 792 | N | GLY | A | 103 | 27.055 | −23.914 | 24.307 | 1.00 | 145.69 | A |
| ATOM | 793 | CA | GLY | A | 103 | 27.063 | −25.329 | 23.974 | 1.00 | 143.01 | A |
| ATOM | 794 | C | GLY | A | 103 | 26.570 | −26.179 | 25.130 | 1.00 | 140.88 | A |
| ATOM | 795 | O | GLY | A | 103 | 26.309 | −27.373 | 24.971 | 1.00 | 135.37 | A |
| ATOM | 796 | N | ALA | A | 104 | 26.446 | −25.557 | 26.298 | 1.00 | 177.61 | A |
| ATOM | 797 | CA | ALA | A | 104 | 25.979 | −26.238 | 27.500 | 1.00 | 177.61 | A |
| ATOM | 798 | CB | ALA | A | 104 | 27.165 | −26.577 | 28.401 | 1.00 | 92.35 | A |
| ATOM | 799 | C | ALA | A | 104 | 24.984 | −25.354 | 28.251 | 1.00 | 177.61 | A |
| ATOM | 800 | O | ALA | A | 104 | 25.350 | −24.282 | 28.734 | 1.00 | 177.61 | A |
| ATOM | 801 | N | GLU | A | 105 | 23.733 | −25.816 | 28.330 | 1.00 | 160.16 | A |
| ATOM | 802 | CA | GLU | A | 105 | 22.630 | −25.120 | 29.009 | 1.00 | 160.16 | A |
| ATOM | 803 | CB | GLU | A | 105 | 23.156 | −24.162 | 30.088 | 1.00 | 196.22 | A |
| ATOM | 804 | CG | GLU | A | 105 | 23.811 | −24.848 | 31.272 | 1.00 | 196.22 | A |
| ATOM | 805 | CD | GLU | A | 105 | 22.859 | −25.773 | 32.004 | 1.00 | 196.22 | A |
| ATOM | 806 | OE1 | GLU | A | 105 | 22.413 | −26.771 | 31.399 | 1.00 | 196.22 | A |
| ATOM | 807 | OE2 | GLU | A | 105 | 22.554 | −25.499 | 33.183 | 1.00 | 194.99 | A |
| ATOM | 808 | C | GLU | A | 105 | 21.694 | −24.354 | 28.070 | 1.00 | 160.16 | A |
| ATOM | 809 | O | GLU | A | 105 | 21.083 | −24.942 | 27.175 | 1.00 | 160.16 | A |
| ATOM | 810 | N | ALA | A | 106 | 21.582 | −23.045 | 28.299 | 1.00 | 163.39 | A |
| ATOM | 811 | CA | ALA | A | 106 | 20.733 | −22.144 | 27.514 | 1.00 | 163.39 | A |
| ATOM | 812 | CB | ALA | A | 106 | 20.711 | −22.565 | 26.042 | 1.00 | 98.03 | A |
| ATOM | 813 | C | ALA | A | 106 | 19.309 | −22.084 | 28.062 | 1.00 | 163.39 | A |
| ATOM | 814 | O | ALA | A | 106 | 18.412 | −22.762 | 27.560 | 1.00 | 163.39 | A |
| ATOM | 815 | N | LYS | A | 107 | 19.113 | −21.265 | 29.094 | 1.00 | 120.55 | A |
| ATOM | 816 | CA | LYS | A | 107 | 17.806 | −21.094 | 29.729 | 1.00 | 114.67 | A |
| ATOM | 817 | CB | LYS | A | 107 | 17.943 | −20.286 | 31.029 | 1.00 | 188.28 | A |
| ATOM | 818 | CG | LYS | A | 107 | 18.523 | −21.038 | 32.225 | 1.00 | 188.23 | A |
| ATOM | 819 | CD | LYS | A | 107 | 19.968 | −21.458 | 32.010 | 1.00 | 189.66 | A |
| ATOM | 820 | CE | LYS | A | 107 | 20.567 | −22.020 | 33.292 | 1.00 | 185.70 | A |
| ATOM | 821 | NZ | LYS | A | 107 | 19.752 | −23.133 | 33.855 | 1.00 | 185.96 | A |
| ATOM | 822 | C | LYS | A | 107 | 16.817 | −20.381 | 28.806 | 1.00 | 110.15 | A |
| ATOM | 823 | O | LYS | A | 107 | 17.200 | −19.842 | 27.768 | 1.00 | 109.28 | A |
| ATOM | 824 | N | PRO | A | 108 | 15.524 | −20.379 | 29.174 | 1.00 | 102.02 | A |
| ATOM | 825 | CD | PRO | A | 108 | 14.896 | −21.146 | 30.268 | 1.00 | 97.80 | A |
| ATOM | 826 | CA | PRO | A | 108 | 14.502 | −19.718 | 28.360 | 1.00 | 100.76 | A |
| ATOM | 827 | CB | PRO | A | 108 | 13.222 | −20.430 | 28.780 | 1.00 | 96.95 | A |
| ATOM | 828 | CG | PRO | A | 108 | 13.455 | −20.656 | 30.231 | 1.00 | 94.33 | A |
| ATOM | 829 | C | PRO | A | 108 | 14.457 | −18.225 | 28.672 | 1.00 | 99.99 | A |
| ATOM | 830 | O | PRO | A | 108 | 14.923 | −17.795 | 29.725 | 1.00 | 98.17 | A |
| ATOM | 831 | N | TRP | A | 109 | 13.910 | −17.440 | 27.749 | 1.00 | 68.84 | A |
| ATOM | 832 | CA | TRP | A | 109 | 13.796 | −15.999 | 27.937 | 1.00 | 66.29 | A |
| ATOM | 833 | CB | TRP | A | 109 | 14.799 | −15.248 | 27.065 | 1.00 | 71.98 | A |
| ATOM | 834 | CG | TRP | A | 109 | 14.709 | −15.572 | 25.605 | 1.00 | 71.67 | A |
| ATOM | 835 | CD2 | TRP | A | 109 | 13.802 | −15.006 | 24.644 | 1.00 | 70.68 | A |
| ATOM | 836 | CE2 | TRP | A | 109 | 14.098 | −15.593 | 23.396 | 1.00 | 69.93 | A |
| ATOM | 837 | CE3 | TRP | A | 109 | 12.771 | −14.062 | 24.717 | 1.00 | 70.78 | A |
| ATOM | 838 | CD1 | TRP | A | 109 | 15.484 | −16.453 | 24.919 | 1.00 | 68.77 | A |
| ATOM | 839 | NE1 | TRP | A | 109 | 15.128 | −16.471 | 23.594 | 1.00 | 67.09 | A |
| ATOM | 840 | CZ2 | TRP | A | 109 | 13.400 | −15.269 | 22.228 | 1.00 | 66.38 | A |
| ATOM | 841 | CZ3 | TRP | A | 109 | 12.076 | −13.740 | 23.553 | 1.00 | 70.54 | A |
| ATOM | 842 | CH2 | TRP | A | 109 | 12.397 | −14.344 | 22.325 | 1.00 | 66.18 | A |
| ATOM | 843 | C | TRP | A | 109 | 12.393 | −15.494 | 27.623 | 1.00 | 67.90 | A |
| ATOM | 844 | O | TRP | A | 109 | 11.714 | −15.997 | 26.720 | 1.00 | 69.51 | A |
| ATOM | 845 | N | TYR | A | 110 | 11.970 | −14.493 | 28.386 | 1.00 | 56.87 | A |
| ATOM | 846 | CA | TYR | A | 110 | 10.658 | −13.889 | 28.212 | 1.00 | 55.51 | A |
| ATOM | 847 | CB | TYR | A | 110 | 9.801 | −14.094 | 29.458 | 1.00 | 67.80 | A |
| ATOM | 848 | CG | TYR | A | 110 | 9.486 | −15.540 | 29.772 | 1.00 | 71.35 | A |
| ATOM | 849 | CD1 | TYR | A | 110 | 10.466 | −16.401 | 30.273 | 1.00 | 75.44 | A |
| ATOM | 850 | CE1 | TYR | A | 110 | 10.159 | −17.718 | 30.610 | 1.00 | 75.76 | A |
| ATOM | 851 | CD2 | TYR | A | 110 | 8.194 | −16.035 | 29.608 | 1.00 | 71.58 | A |
| ATOM | 852 | CE2 | TYR | A | 110 | 7.877 | −17.345 | 29.938 | 1.00 | 76.95 | A |
| ATOM | 853 | CZ | TYR | A | 110 | 8.859 | −18.180 | 30.441 | 1.00 | 77.24 | A |
| ATOM | 854 | OH | TYR | A | 110 | 8.533 | −19.471 | 30.786 | 1.00 | 78.44 | A |
| ATOM | 855 | C | TYR | A | 110 | 10.831 | −12.401 | 27.959 | 1.00 | 54.02 | A |
| ATOM | 856 | O | TYR | A | 110 | 11.648 | −11.743 | 28.614 | 1.00 | 55.21 | A |
| ATOM | 857 | N | GLU | A | 111 | 10.062 | −11.881 | 27.004 | 1.00 | 55.92 | A |
| ATOM | 858 | CA | GLU | A | 111 | 10.128 | −10.473 | 26.654 | 1.00 | 53.94 | A |
| ATOM | 859 | CB | GLU | A | 111 | 11.086 | −10.285 | 25.484 | 1.00 | 86.90 | A |
| ATOM | 860 | CG | GLU | A | 111 | 11.970 | −9.071 | 25.618 | 1.00 | 93.82 | A |
| ATOM | 861 | CD | GLU | A | 111 | 12.855 | −9.142 | 26.844 | 1.00 | 98.79 | A |
| ATOM | 862 | OE1 | GLU | A | 111 | 13.638 | −10.109 | 26.942 | 1.00 | 99.00 | A |
| ATOM | 863 | OE2 | GLU | A | 111 | 12.769 | −8.236 | 27.706 | 1.00 | 105.95 | A |
| ATOM | 864 | C | GLU | A | 111 | 8.747 | −9.934 | 26.286 | 1.00 | 54.52 | A |
| ATOM | 865 | O | GLU | A | 111 | 8.196 | −10.282 | 25.244 | 1.00 | 59.88 | A |
| ATOM | 866 | N | PRO | A | 112 | 8.156 | −9.096 | 27.157 | 1.00 | 53.66 | A |
| ATOM | 867 | CD | PRO | A | 112 | 8.528 | −8.897 | 28.572 | 1.00 | 47.03 | A |
| ATOM | 868 | CA | PRO | A | 112 | 6.832 | −8.518 | 26.894 | 1.00 | 52.89 | A |
| ATOM | 869 | CB | PRO | A | 112 | 6.218 | −8.447 | 28.285 | 1.00 | 43.10 | A |
| ATOM | 870 | CG | PRO | A | 112 | 7.389 | −8.007 | 29.096 | 1.00 | 44.23 | A |
| ATOM | 871 | C | PRO | A | 112 | 6.940 | −7.128 | 26.255 | 1.00 | 49.92 | A |
| ATOM | 872 | O | PRO | A | 112 | 7.948 | −6.443 | 26.410 | 1.00 | 50.05 | A |
| ATOM | 873 | N | ILE | A | 113 | 5.898 | −6.726 | 25.533 | 1.00 | 44.34 | A |
| ATOM | 874 | CA | ILE | A | 113 | 5.854 | −5.417 | 24.900 | 1.00 | 46.31 | A |
| ATOM | 875 | CB | ILE | A | 113 | 6.202 | −5.486 | 23.417 | 1.00 | 47.28 | A |
| ATOM | 876 | CG2 | ILE | A | 113 | 6.081 | −4.101 | 22.799 | 1.00 | 43.30 | A |
| ATOM | 877 | CG1 | ILE | A | 113 | 7.621 | −6.011 | 23.228 | 1.00 | 44.34 | A |
| ATOM | 878 | CD1 | ILE | A | 113 | 8.088 | −5.963 | 21.769 | 1.00 | 44.82 | A |
| ATOM | 879 | C | ILE | A | 113 | 4.451 | −4.845 | 24.998 | 1.00 | 50.53 | A |
| ATOM | 880 | O | ILE | A | 113 | 3.489 | −5.491 | 24.590 | 1.00 | 53.93 | A |
| ATOM | 881 | N | TYR | A | 114 | 4.331 | −3.633 | 25.527 | 1.00 | 42.91 | A |
| ATOM | 882 | CA | TYR | A | 114 | 3.023 | −2.994 | 25.649 | 1.00 | 41.19 | A |
| ATOM | 883 | CB | TYR | A | 114 | 2.769 | −2.540 | 27.090 | 1.00 | 33.54 | A |
| ATOM | 884 | CG | TYR | A | 114 | 1.358 | −2.068 | 27.332 | 1.00 | 31.40 | A |
| ATOM | 885 | CD1 | TYR | A | 114 | 0.337 | −2.974 | 27.599 | 1.00 | 35.69 | A |
| ATOM | 886 | CE1 | TYR | A | 114 | −0.978 | −2.541 | 27.802 | 1.00 | 35.26 | A |
| ATOM | 887 | CD2 | TYR | A | 114 | 1.036 | −0.714 | 27.271 | 1.00 | 31.57 | A |
| ATOM | 888 | CE2 | TYR | A | 114 | −0.273 | −0.272 | 27.467 | 1.00 | 35.90 | A |
| ATOM | 889 | CZ | TYR | A | 114 | −1.275 | −1.190 | 27.731 | 1.00 | 35.98 | A |
| ATOM | 890 | OH | TYR | A | 114 | −2.574 | −0.757 | 27.899 | 1.00 | 35.48 | A |
| ATOM | 891 | C | TYR | A | 114 | 2.950 | −1.790 | 24.725 | 1.00 | 41.03 | A |
| ATOM | 892 | O | TYR | A | 114 | 3.934 | −1.072 | 24.543 | 1.00 | 43.94 | A |
| ATOM | 893 | N | LEU | A | 115 | 1.772 | −1.564 | 24.164 | 1.00 | 37.66 | A |
| ATOM | 894 | CA | LEU | A | 115 | 1.551 | −0.459 | 23.244 | 1.00 | 36.70 | A |
| ATOM | 895 | CB | LEU | A | 115 | 1.543 | −0.994 | 21.806 | 1.00 | 41.07 | A |
| ATOM | 896 | CG | LEU | A | 115 | 2.680 | −0.685 | 20.828 | 1.00 | 44.32 | A |
| ATOM | 897 | CD1 | LEU | A | 115 | 4.030 | −0.675 | 21.529 | 1.00 | 42.70 | A |
| ATOM | 898 | CD2 | LEU | A | 115 | 2.637 | −1.706 | 19.711 | 1.00 | 47.62 | A |
| ATOM | 899 | C | LEU | A | 115 | 0.208 | 0.173 | 23.560 | 1.00 | 39.14 | A |
| ATOM | 900 | O | LEU | A | 115 | −0.757 | −0.543 | 23.828 | 1.00 | 41.67 | A |
| ATOM | 901 | N | GLY | A | 116 | 0.134 | 1.503 | 23.523 | 1.00 | 42.26 | A |
| ATOM | 902 | CA | GLY | A | 116 | −1.128 | 2.162 | 23.806 | 1.00 | 36.60 | A |
| ATOM | 903 | C | GLY | A | 116 | −1.142 | 3.656 | 23.603 | 1.00 | 40.45 | A |
| ATOM | 904 | O | GLY | A | 116 | −0.182 | 4.331 | 23.919 | 1.00 | 39.33 | A |
| ATOM | 905 | N | GLY | A | 117 | −2.242 | 4.172 | 23.070 | 1.00 | 37.25 | A |
| ATOM | 906 | CA | GLY | A | 117 | −2.377 | 5.601 | 22.839 | 1.00 | 40.25 | A |
| ATOM | 907 | C | GLY | A | 117 | −3.783 | 5.970 | 22.389 | 1.00 | 41.56 | A |
| ATOM | 908 | O | GLY | A | 117 | −4.591 | 5.099 | 22.070 | 1.00 | 42.94 | A |
| ATOM | 909 | N | VAL | A | 118 | −4.079 | 7.263 | 22.356 | 1.00 | 39.42 | A |
| ATOM | 910 | CA | VAL | A | 118 | −5.396 | 7.733 | 21.942 | 1.00 | 42.20 | A |
| ATOM | 911 | CB | VAL | A | 118 | −5.867 | 8.854 | 22.880 | 1.00 | 31.82 | A |
| ATOM | 912 | CG1 | VAL | A | 118 | −7.199 | 9.412 | 22.421 | 1.00 | 33.22 | A |
| ATOM | 913 | CG2 | VAL | A | 118 | −5.974 | 8.311 | 24.288 | 1.00 | 31.88 | A |
| ATOM | 914 | C | VAL | A | 118 | −5.355 | 8.233 | 20.495 | 1.00 | 40.11 | A |
| ATOM | 915 | O | VAL | A | 118 | −4.407 | 8.916 | 20.104 | 1.00 | 44.06 | A |
| ATOM | 916 | N | PHE | A | 119 | −6.371 | 7.886 | 19.701 | 1.00 | 45.47 | A |
| ATOM | 917 | CA | PHE | A | 119 | −6.410 | 8.286 | 18.291 | 1.00 | 46.78 | A |
| ATOM | 918 | CB | PHE | A | 119 | −5.855 | 7.167 | 17.402 | 1.00 | 34.22 | A |
| ATOM | 919 | CG | PHE | A | 119 | −4.417 | 6.827 | 17.680 | 1.00 | 31.82 | A |
| ATOM | 920 | CD1 | PHE | A | 119 | −4.073 | 5.953 | 18.711 | 1.00 | 30.01 | A |
| ATOM | 921 | CD2 | PHE | A | 119 | −3.396 | 7.410 | 16.934 | 1.00 | 30.50 | A |
| ATOM | 922 | CE1 | PHE | A | 119 | −2.727 | 5.665 | 18.998 | 1.00 | 31.08 | A |
| ATOM | 923 | CE2 | PHE | A | 119 | −2.051 | 7.133 | 17.207 | 1.00 | 31.31 | A |
| ATOM | 924 | CZ | PHE | A | 119 | −1.713 | 6.258 | 18.243 | 1.00 | 30.40 | A |
| ATOM | 925 | C | PHE | A | 119 | −7.782 | 8.671 | 17.765 | 1.00 | 47.69 | A |
| ATOM | 926 | O | PHE | A | 119 | −8.800 | 8.189 | 18.262 | 1.00 | 43.79 | A |
| ATOM | 927 | N | GLN | A | 120 | −7.792 | 9.551 | 16.757 | 1.00 | 37.20 | A |
| ATOM | 928 | CA | GLN | A | 120 | −9.020 | 10.013 | 16.102 | 1.00 | 40.57 | A |
| ATOM | 929 | CB | GLN | A | 120 | −8.800 | 11.377 | 15.455 | 1.00 | 74.60 | A |
| ATOM | 930 | CG | GLN | A | 120 | −9.730 | 12.477 | 15.929 | 1.00 | 74.75 | A |
| ATOM | 931 | CD | GLN | A | 120 | −11.199 | 12.111 | 15.804 | 1.00 | 74.35 | A |
| ATOM | 932 | OE1 | GLN | A | 120 | −11.615 | 11.514 | 14.814 | 1.00 | 73.91 | A |
| ATOM | 933 | NE2 | GLN | A | 120 | −11.998 | 12.486 | 16.805 | 1.00 | 77.18 | A |
| ATOM | 934 | C | GLN | A | 120 | −9.268 | 8.983 | 15.007 | 1.00 | 39.71 | A |
| ATOM | 935 | O | GLN | A | 120 | −8.412 | 8.790 | 14.144 | 1.00 | 42.41 | A |
| ATOM | 936 | N | LEU | A | 121 | −10.417 | 8.313 | 15.040 | 1.00 | 44.31 | A |
| ATOM | 937 | CA | LEU | A | 121 | −10.741 | 7.291 | 14.035 | 1.00 | 45.97 | A |
| ATOM | 938 | CB | LEU | A | 121 | −10.843 | 5.930 | 14.715 | 1.00 | 40.24 | A |
| ATOM | 939 | CG | LEU | A | 121 | −9.595 | 5.055 | 14.804 | 1.00 | 38.51 | A |
| ATOM | 940 | CD1 | LEU | A | 121 | −8.347 | 5.893 | 14.838 | 1.00 | 39.90 | A |
| ATOM | 941 | CD2 | LEU | A | 121 | −9.705 | 4.156 | 16.024 | 1.00 | 34.00 | A |
| ATOM | 942 | C | LEU | A | 121 | −12.040 | 7.568 | 13.277 | 1.00 | 48.01 | A |
| ATOM | 943 | O | LEU | A | 121 | −12.964 | 8.179 | 13.814 | 1.00 | 53.28 | A |
| ATOM | 944 | N | GLU | A | 122 | −12.099 | 7.112 | 12.028 | 1.00 | 43.96 | A |
| ATOM | 945 | CA | GLU | A | 122 | −13.281 | 7.278 | 11.170 | 1.00 | 45.50 | A |
| ATOM | 946 | CB | GLU | A | 122 | −12.890 | 7.462 | 9.702 | 1.00 | 98.06 | A |
| ATOM | 947 | CG | GLU | A | 122 | −12.028 | 8.645 | 9.331 | 1.00 | 101.10 | A |
| ATOM | 948 | CD | GLU | A | 122 | −11.636 | 8.607 | 7.850 | 1.00 | 106.38 | A |
| ATOM | 949 | OE1 | GLU | A | 122 | −10.951 | 7.644 | 7.431 | 1.00 | 107.88 | A |
| ATOM | 950 | OE2 | GLU | A | 122 | −12.022 | 9.533 | 7.103 | 1.00 | 106.47 | A |
| ATOM | 951 | C | GLU | A | 122 | −14.133 | 6.012 | 11.214 | 1.00 | 44.30 | A |
| ATOM | 952 | O | GLU | A | 122 | −13.620 | 4.906 | 11.406 | 1.00 | 45.49 | A |
| ATOM | 953 | N | LYS | A | 123 | −15.435 | 6.157 | 11.009 | 1.00 | 56.37 | A |
| ATOM | 954 | CA | LYS | A | 123 | −16.290 | 4.984 | 10.975 | 1.00 | 57.22 | A |
| ATOM | 955 | CB | LYS | A | 123 | −17.735 | 5.377 | 10.701 | 1.00 | 72.03 | A |
| ATOM | 956 | CG | LYS | A | 123 | −18.631 | 4.187 | 10.429 | 1.00 | 68.19 | A |
| ATOM | 957 | CD | LYS | A | 123 | −20.044 | 4.621 | 10.099 | 1.00 | 65.86 | A |
| ATOM | 958 | CE | LYS | A | 123 | −20.920 | 3.422 | 9.784 | 1.00 | 67.59 | A |
| ATOM | 959 | NZ | LYS | A | 123 | −22.278 | 3.846 | 9.353 | 1.00 | 71.60 | A |
| ATOM | 960 | C | LYS | A | 123 | −15.779 | 4.122 | 9.823 | 1.00 | 58.04 | A |
| ATOM | 961 | O | LYS | A | 123 | −15.631 | 4.605 | 8.701 | 1.00 | 62.80 | A |
| ATOM | 962 | N | GLY | A | 124 | −15.482 | 2.859 | 10.104 | 1.00 | 47.03 | A |
| ATOM | 963 | CA | GLY | A | 124 | −15.006 | 1.970 | 9.065 | 1.00 | 48.14 | A |
| ATOM | 964 | C | GLY | A | 124 | −13.527 | 1.659 | 9.122 | 1.00 | 49.55 | A |
| ATOM | 965 | O | GLY | A | 124 | −13.074 | 0.747 | 8.436 | 1.00 | 50.35 | A |
| ATOM | 966 | N | ASP | A | 125 | −12.762 | 2.399 | 9.921 | 1.00 | 43.64 | A |
| ATOM | 967 | CA | ASP | A | 125 | −11.320 | 2.138 | 10.017 | 1.00 | 42.43 | A |
| ATOM | 968 | CB | ASP | A | 125 | −10.638 | 3.162 | 10.935 | 1.00 | 63.70 | A |
| ATOM | 969 | CG | ASP | A | 125 | −10.581 | 4.558 | 10.328 | 1.00 | 69.16 | A |
| ATOM | 970 | OD1 | ASP | A | 125 | −10.876 | 4.703 | 9.123 | 1.00 | 70.81 | A |
| ATOM | 971 | OD2 | ASP | A | 125 | −10.231 | 5.509 | 11.063 | 1.00 | 67.82 | A |
| ATOM | 972 | C | ASP | A | 125 | −11.021 | 0.723 | 10.524 | 1.00 | 41.48 | A |
| ATOM | 973 | O | ASP | A | 125 | −11.793 | 0.150 | 11.292 | 1.00 | 38.39 | A |
| ATOM | 974 | N | ARG | A | 126 | −9.897 | 0.171 | 10.083 | 1.00 | 47.91 | A |
| ATOM | 975 | CA | ARG | A | 126 | −9.482 | −1.167 | 10.473 | 1.00 | 43.39 | A |
| ATOM | 976 | CB | ARG | A | 126 | −9.232 | −2.027 | 9.229 | 1.00 | 66.97 | A |
| ATOM | 977 | CG | ARG | A | 126 | −10.467 | −2.410 | 8.437 | 1.00 | 65.46 | A |
| ATOM | 978 | CD | ARG | A | 126 | −10.067 | −3.058 | 7.115 | 1.00 | 70.38 | A |
| ATOM | 979 | NE | ARG | A | 126 | −11.189 | −3.755 | 6.496 | 1.00 | 73.93 | A |
| ATOM | 980 | CZ | ARG | A | 126 | −11.639 | −4.955 | 6.877 | 1.00 | 77.16 | A |
| ATOM | 981 | NH1 | ARG | A | 126 | −12.678 | −5.513 | 6.257 | 1.00 | 76.83 | A |
| ATOM | 982 | NH2 | ARG | A | 126 | −11.039 | −5.612 | 7.865 | 1.00 | 75.35 | A |
| ATOM | 983 | C | ARG | A | 126 | −8.195 | −1.085 | 11.287 | 1.00 | 44.23 | A |
| ATOM | 984 | O | ARG | A | 126 | −7.214 | −0.489 | 10.845 | 1.00 | 41.45 | A |
| ATOM | 985 | N | LEU | A | 127 | −8.185 | −1.686 | 12.472 | 1.00 | 46.87 | A |
| ATOM | 986 | CA | LEU | A | 127 | −6.987 | −1.654 | 13.298 | 1.00 | 43.25 | A |
| ATOM | 987 | CB | LEU | A | 127 | −7.340 | −1.170 | 14.704 | 1.00 | 48.60 | A |
| ATOM | 988 | CG | LEU | A | 127 | −7.934 | 0.236 | 14.740 | 1.00 | 47.58 | A |
| ATOM | 989 | CD1 | LEU | A | 127 | −8.549 | 0.505 | 16.091 | 1.00 | 48.16 | A |
| ATOM | 990 | CD2 | LEU | A | 127 | −6.852 | 1.244 | 14.435 | 1.00 | 48.24 | A |
| ATOM | 991 | C | LEU | A | 127 | −6.363 | −3.037 | 13.348 | 1.00 | 43.00 | A |
| ATOM | 992 | O | LEU | A | 127 | −7.072 | −4.033 | 13.465 | 1.00 | 39.20 | A |
| ATOM | 993 | N | SER | A | 128 | −5.039 | −3.090 | 13.245 | 1.00 | 45.82 | A |
| ATOM | 994 | CA | SER | A | 128 | −4.314 | −4.354 | 13.265 | 1.00 | 45.49 | A |
| ATOM | 995 | CB | SER | A | 128 | −3.617 | −4.591 | 11.928 | 1.00 | 89.30 | A |
| ATOM | 996 | OG | SER | A | 128 | −4.445 | −4.210 | 10.850 | 1.00 | 95.15 | A |
| ATOM | 997 | C | SER | A | 128 | −3.252 | −4.340 | 14.360 | 1.00 | 44.67 | A |
| ATOM | 998 | O | SER | A | 128 | −2.601 | −3.321 | 14.610 | 1.00 | 45.95 | A |
| ATOM | 999 | N | ALA | A | 129 | −3.084 | −5.479 | 15.016 | 1.00 | 55.08 | A |
| ATOM | 1000 | CA | ALA | A | 129 | −2.083 | −5.627 | 16.060 | 1.00 | 56.05 | A |
| ATOM | 1001 | CB | ALA | A | 129 | −2.758 | −5.896 | 17.408 | 1.00 | 50.49 | A |
| ATOM | 1002 | C | ALA | A | 129 | −1.266 | −6.829 | 15.606 | 1.00 | 54.82 | A |
| ATOM | 1003 | O | ALA | A | 129 | −1.675 | −7.975 | 15.796 | 1.00 | 55.70 | A |
| ATOM | 1004 | N | GLU | A | 130 | −0.119 | −6.564 | 14.990 | 1.00 | 57.27 | A |
| ATOM | 1005 | CA | GLU | A | 130 | 0.721 | −7.632 | 14.468 | 1.00 | 58.24 | A |
| ATOM | 1006 | CB | GLU | A | 130 | 0.821 | −7.479 | 12.952 | 1.00 | 110.53 | A |
| ATOM | 1007 | CG | GLU | A | 130 | −0.533 | −7.522 | 12.263 | 1.00 | 110.07 | A |
| ATOM | 1008 | CD | GLU | A | 130 | −0.527 | −6.873 | 10.888 | 1.00 | 115.56 | A |
| ATOM | 1009 | OE1 | GLU | A | 130 | −1.580 | −6.917 | 10.213 | 1.00 | 116.93 | A |
| ATOM | 1010 | OE2 | GLU | A | 130 | 0.518 | −6.314 | 10.482 | 1.00 | 115.37 | A |
| ATOM | 1011 | C | GLU | A | 130 | 2.117 | −7.731 | 15.079 | 1.00 | 54.63 | A |
| ATOM | 1012 | O | GLU | A | 130 | 2.598 | −6.804 | 15.725 | 1.00 | 54.55 | A |
| ATOM | 1013 | N | ILE | A | 131 | 2.748 | −8.886 | 14.887 | 1.00 | 55.18 | A |
| ATOM | 1014 | CA | ILE | A | 131 | 4.104 | −9.136 | 15.368 | 1.00 | 57.72 | A |
| ATOM | 1015 | CB | ILE | A | 131 | 4.129 | −10.116 | 16.541 | 1.00 | 59.77 | A |
| ATOM | 1016 | CG2 | ILE | A | 131 | 3.477 | −9.477 | 17.758 | 1.00 | 60.00 | A |
| ATOM | 1017 | CG1 | ILE | A | 131 | 3.441 | −11.421 | 16.130 | 1.00 | 55.96 | A |
| ATOM | 1018 | CD1 | ILE | A | 131 | 3.482 | −12.491 | 17.194 | 1.00 | 54.57 | A |
| ATOM | 1019 | C | ILE | A | 131 | 4.866 | −9.761 | 14.216 | 1.00 | 59.61 | A |
| ATOM | 1020 | O | ILE | A | 131 | 4.254 | −10.268 | 13.278 | 1.00 | 60.33 | A |
| ATOM | 1021 | N | ASN | A | 132 | 6.191 | −9.740 | 14.290 | 1.00 | 63.24 | A |
| ATOM | 1022 | CA | ASN | A | 132 | 7.015 | −10.295 | 13.223 | 1.00 | 60.24 | A |
| ATOM | 1023 | CB | ASN | A | 132 | 8.245 | −9.422 | 13.027 | 1.00 | 54.69 | A |
| ATOM | 1024 | CG | ASN | A | 132 | 9.167 | −9.468 | 14.210 | 1.00 | 53.39 | A |
| ATOM | 1025 | OD1 | ASN | A | 132 | 8.725 | −9.449 | 15.359 | 1.00 | 55.08 | A |
| ATOM | 1026 | ND2 | ASN | A | 132 | 10.462 | −9.524 | 13.943 | 1.00 | 51.22 | A |
| ATOM | 1027 | C | ASN | A | 132 | 7.452 | −11.744 | 13.456 | 1.00 | 57.89 | A |
| ATOM | 1028 | O | ASN | A | 132 | 7.698 | −12.473 | 12.495 | 1.00 | 61.06 | A |
| ATOM | 1029 | N | ARG | A | 133 | 7.551 | −12.164 | 14.718 | 1.00 | 56.81 | A |
| ATOM | 1030 | CA | ARG | A | 133 | 7.967 | −13.535 | 15.030 | 1.00 | 52.40 | A |
| ATOM | 1031 | CB | ARG | A | 133 | 9.280 | −13.518 | 15.821 | 1.00 | 71.03 | A |
| ATOM | 1032 | CG | ARG | A | 133 | 10.450 | −12.904 | 15.073 | 1.00 | 73.61 | A |
| ATOM | 1033 | CD | ARG | A | 133 | 10.704 | −13.621 | 13.752 | 1.00 | 78.85 | A |
| ATOM | 1034 | NE | ARG | A | 133 | 10.997 | −15.043 | 13.935 | 1.00 | 76.71 | A |
| ATOM | 1035 | CZ | ARG | A | 133 | 12.131 | −15.520 | 14.440 | 1.00 | 77.81 | A |
| ATOM | 1036 | NH1 | ARG | A | 133 | 12.305 | −16.828 | 14.571 | 1.00 | 80.49 | A |
| ATOM | 1037 | NH2 | ARG | A | 133 | 13.097 | −14.691 | 14.805 | 1.00 | 77.10 | A |
| ATOM | 1038 | C | ARG | A | 133 | 6.920 | −14.338 | 15.803 | 1.00 | 56.99 | A |
| ATOM | 1039 | O | ARG | A | 133 | 7.143 | −14.701 | 16.954 | 1.00 | 59.35 | A |
| ATOM | 1040 | N | PRO | A | 134 | 5.771 | −14.647 | 15.170 | 1.00 | 48.15 | A |
| ATOM | 1041 | CD | PRO | A | 134 | 5.451 | −14.444 | 13.745 | 1.00 | 40.46 | A |
| ATOM | 1042 | CA | PRO | A | 134 | 4.704 | −15.414 | 15.827 | 1.00 | 51.87 | A |
| ATOM | 1043 | CB | PRO | A | 134 | 3.644 | −15.542 | 14.738 | 1.00 | 43.18 | A |
| ATOM | 1044 | CG | PRO | A | 134 | 4.459 | −15.556 | 13.486 | 1.00 | 45.18 | A |
| ATOM | 1045 | C | PRO | A | 134 | 5.157 | −16.767 | 16.341 | 1.00 | 57.05 | A |
| ATOM | 1046 | O | PRO | A | 134 | 4.445 | −17.412 | 17.091 | 1.00 | 60.09 | A |
| ATOM | 1047 | N | ASP | A | 135 | 6.343 | −17.199 | 15.938 | 1.00 | 73.14 | A |
| ATOM | 1048 | CA | ASP | A | 135 | 6.858 | −18.479 | 16.395 | 1.00 | 69.60 | A |
| ATOM | 1049 | CB | ASP | A | 135 | 7.953 | −18.973 | 15.443 | 1.00 | 117.53 | A |
| ATOM | 1050 | CG | ASP | A | 135 | 8.905 | −17.873 | 15.028 | 1.00 | 117.00 | A |
| ATOM | 1051 | OD1 | ASP | A | 135 | 9.747 | −17.473 | 15.857 | 1.00 | 112.94 | A |
| ATOM | 1052 | OD2 | ASP | A | 135 | 8.806 | −17.403 | 13.873 | 1.00 | 118.58 | A |
| ATOM | 1053 | C | ASP | A | 135 | 7.379 | −18.389 | 17.832 | 1.00 | 68.31 | A |
| ATOM | 1054 | O | ASP | A | 135 | 7.635 | −19.413 | 18.469 | 1.00 | 75.00 | A |
| ATOM | 1055 | N | TYR | A | 136 | 7.519 | −17.165 | 18.344 | 1.00 | 53.02 | A |
| ATOM | 1056 | CA | TYR | A | 136 | 7.993 | −16.945 | 19.712 | 1.00 | 54.23 | A |
| ATOM | 1057 | CB | TYR | A | 136 | 9.078 | −15.869 | 19.748 | 1.00 | 82.74 | A |
| ATOM | 1058 | CG | TYR | A | 136 | 10.433 | −16.299 | 19.241 | 1.00 | 82.35 | A |
| ATOM | 1059 | CD1 | TYR | A | 136 | 11.023 | −15.657 | 18.154 | 1.00 | 81.95 | A |
| ATOM | 1060 | CE1 | TYR | A | 136 | 12.296 | −16.009 | 17.709 | 1.00 | 84.50 | A |
| ATOM | 1061 | CD2 | TYR | A | 136 | 11.147 | −17.313 | 19.873 | 1.00 | 84.52 | A |
| ATOM | 1062 | CE2 | TYR | A | 136 | 12.421 | −17.677 | 19.437 | 1.00 | 83.99 | A |
| ATOM | 1063 | CZ | TYR | A | 136 | 12.988 | −17.018 | 18.355 | 1.00 | 85.29 | A |
| ATOM | 1064 | OH | TYR | A | 136 | 14.248 | −17.357 | 17.923 | 1.00 | 89.13 | A |
| ATOM | 1065 | C | TYR | A | 136 | 6.852 | −16.505 | 20.619 | 1.00 | 56.14 | A |
| ATOM | 1066 | O | TYR | A | 136 | 7.063 | −16.186 | 21.788 | 1.00 | 59.02 | A |
| ATOM | 1067 | N | LEU | A | 137 | 5.645 | −16.493 | 20.074 | 1.00 | 65.58 | A |
| ATOM | 1068 | CA | LEU | A | 137 | 4.476 | −16.074 | 20.825 | 1.00 | 62.29 | A |
| ATOM | 1069 | CB | LEU | A | 137 | 3.272 | −16.008 | 19.897 | 1.00 | 61.71 | A |
| ATOM | 1070 | CG | LEU | A | 137 | 2.581 | −14.657 | 19.747 | 1.00 | 59.44 | A |
| ATOM | 1071 | CD1 | LEU | A | 137 | 1.272 | −14.870 | 19.011 | 1.00 | 61.58 | A |
| ATOM | 1072 | CD2 | LEU | A | 137 | 2.309 | −14.041 | 21.104 | 1.00 | 63.20 | A |
| ATOM | 1073 | C | LEU | A | 137 | 4.130 | −16.978 | 22.003 | 1.00 | 61.66 | A |
| ATOM | 1074 | O | LEU | A | 137 | 3.811 | −18.153 | 21.809 | 1.00 | 60.51 | A |
| ATOM | 1075 | N | LEU | A | 138 | 4.186 | −16.436 | 23.222 | 1.00 | 69.22 | A |
| ATOM | 1076 | CA | LEU | A | 138 | 3.818 | −17.220 | 24.407 | 1.00 | 67.02 | A |
| ATOM | 1077 | CB | LEU | A | 138 | 4.774 | −16.997 | 25.590 | 1.00 | 56.18 | A |
| ATOM | 1078 | CG | LEU | A | 138 | 4.220 | −17.599 | 26.905 | 1.00 | 52.30 | A |
| ATOM | 1079 | CD1 | LEU | A | 138 | 3.762 | −19.021 | 26.660 | 1.00 | 53.87 | A |
| ATOM | 1080 | CD2 | LEU | A | 138 | 5.257 | −17.575 | 28.011 | 1.00 | 55.22 | A |
| ATOM | 1081 | C | LEU | A | 138 | 2.409 | −16.878 | 24.869 | 1.00 | 70.37 | A |
| ATOM | 1082 | O | LEU | A | 138 | 2.203 | −15.909 | 25.597 | 1.00 | 69.52 | A |
| ATOM | 1083 | N | PHE | A | 139 | 1.446 | −17.683 | 24.443 | 1.00 | 45.99 | A |
| ATOM | 1084 | CA | PHE | A | 139 | 0.067 | −17.477 | 24.827 | 1.00 | 47.37 | A |
| ATOM | 1085 | CB | PHE | A | 139 | −0.808 | −17.252 | 23.582 | 1.00 | 64.78 | A |
| ATOM | 1086 | CG | PHE | A | 139 | −0.531 | −18.199 | 22.450 | 1.00 | 63.93 | A |
| ATOM | 1087 | CD1 | PHE | A | 139 | −0.749 | −17.798 | 21.137 | 1.00 | 63.46 | A |
| ATOM | 1088 | CD2 | PHE | A | 139 | −0.068 | −19.490 | 22.690 | 1.00 | 66.54 | A |
| ATOM | 1089 | CE1 | PHE | A | 139 | −0.510 | −18.666 | 20.079 | 1.00 | 68.10 | A |
| ATOM | 1090 | CE2 | PHE | A | 139 | 0.174 | −20.367 | 21.641 | 1.00 | 64.08 | A |
| ATOM | 1091 | CZ | PHE | A | 139 | −0.047 | −19.956 | 20.330 | 1.00 | 68.02 | A |
| ATOM | 1092 | C | PHE | A | 139 | −0.400 | −18.687 | 25.628 | 1.00 | 51.40 | A |
| ATOM | 1093 | O | PHE | A | 139 | −1.542 | −19.140 | 25.506 | 1.00 | 51.50 | A |
| ATOM | 1094 | N | ALA | A | 140 | 0.498 | −19.195 | 26.466 | 1.00 | 65.81 | A |
| ATOM | 1095 | CA | ALA | A | 140 | 0.211 | −20.361 | 27.284 | 1.00 | 67.11 | A |
| ATOM | 1096 | CB | ALA | A | 140 | 1.509 | −21.026 | 27.716 | 1.00 | 53.15 | A |
| ATOM | 1097 | C | ALA | A | 140 | −0.620 | −20.040 | 28.509 | 1.00 | 67.50 | A |
| ATOM | 1098 | O | ALA | A | 140 | −1.572 | −20.752 | 28.817 | 1.00 | 69.39 | A |
| ATOM | 1099 | N | GLU | A | 141 | −0.258 | −18.972 | 29.211 | 1.00 | 82.80 | A |
| ATOM | 1100 | CA | GLU | A | 141 | −0.969 | −18.604 | 30.423 | 1.00 | 85.98 | A |
| ATOM | 1101 | CB | GLU | A | 141 | −0.169 | −17.575 | 31.228 | 1.00 | 200.84 | A |
| ATOM | 1102 | CG | GLU | A | 141 | −0.780 | −17.237 | 32.588 | 1.00 | 200.84 | A |
| ATOM | 1103 | CD | GLU | A | 141 | −1.850 | −16.155 | 32.519 | 1.00 | 200.84 | A |
| ATOM | 1104 | OE1 | GLU | A | 141 | −2.727 | −16.126 | 33.408 | 1.00 | 200.84 | A |
| ATOM | 1105 | OE2 | GLU | A | 141 | −1.809 | −15.324 | 31.588 | 1.00 | 200.84 | A |
| ATOM | 1106 | C | GLU | A | 141 | −2.376 | −18.089 | 30.185 | 1.00 | 88.08 | A |
| ATOM | 1107 | O | GLU | A | 141 | −3.332 | −18.846 | 30.319 | 1.00 | 91.76 | A |
| ATOM | 1108 | N | SER | A | 142 | −2.522 | −16.818 | 29.825 | 1.00 | 81.98 | A |
| ATOM | 1109 | CA | SER | A | 142 | −3.862 | −16.277 | 29.637 | 1.00 | 84.97 | A |
| ATOM | 1110 | CB | SER | A | 142 | −4.657 | −16.482 | 30.920 | 1.00 | 49.82 | A |
| ATOM | 1111 | OG | SER | A | 142 | −6.033 | −16.316 | 30.690 | 1.00 | 50.70 | A |
| ATOM | 1112 | C | SER | A | 142 | −3.900 | −14.795 | 29.261 | 1.00 | 86.29 | A |
| ATOM | 1113 | O | SER | A | 142 | −4.101 | −14.434 | 28.095 | 1.00 | 91.38 | A |
| ATOM | 1114 | N | GLY | A | 143 | −3.735 | −13.931 | 30.254 | 1.00 | 53.11 | A |
| ATOM | 1115 | CA | GLY | A | 143 | −3.756 | −12.511 | 29.960 | 1.00 | 50.58 | A |
| ATOM | 1116 | C | GLY | A | 143 | −2.387 | −12.006 | 29.535 | 1.00 | 48.65 | A |
| ATOM | 1117 | O | GLY | A | 143 | −1.985 | −10.912 | 29.925 | 1.00 | 50.64 | A |
| ATOM | 1118 | N | GLN | A | 144 | −1.678 | −12.800 | 28.731 | 1.00 | 50.41 | A |
| ATOM | 1119 | CA | GLN | A | 144 | −0.339 | −12.457 | 28.263 | 1.00 | 44.94 | A |
| ATOM | 1120 | CB | GLN | A | 144 | 0.539 | −13.704 | 28.228 | 1.00 | 60.83 | A |
| ATOM | 1121 | CG | GLN | A | 144 | 0.483 | −14.537 | 29.493 | 1.00 | 60.11 | A |
| ATOM | 1122 | CD | GLN | A | 144 | 1.480 | −15.677 | 29.484 | 1.00 | 60.08 | A |
| ATOM | 1123 | OE1 | GLN | A | 144 | 1.593 | −16.422 | 28.507 | 1.00 | 65.65 | A |
| ATOM | 1124 | NE2 | GLN | A | 144 | 2.206 | −15.825 | 30.583 | 1.00 | 58.62 | A |
| ATOM | 1125 | C | GLN | A | 144 | −0.334 | −11.822 | 26.885 | 1.00 | 46.74 | A |
| ATOM | 1126 | O | GLN | A | 144 | 0.606 | −11.132 | 26.518 | 1.00 | 49.05 | A |
| ATOM | 1127 | N | VAL | A | 145 | −1.379 | −12.061 | 26.114 | 1.00 | 40.36 | A |
| ATOM | 1128 | CA | VAL | A | 145 | −1.463 | −11.502 | 24.782 | 1.00 | 38.61 | A |
| ATOM | 1129 | CB | VAL | A | 145 | −1.133 | −12.569 | 23.726 | 1.00 | 32.42 | A |
| ATOM | 1130 | CG1 | VAL | A | 145 | −1.186 | −11.964 | 22.330 | 1.00 | 30.58 | A |
| ATOM | 1131 | CG2 | VAL | A | 145 | 0.238 | −13.134 | 23.985 | 1.00 | 29.50 | A |
| ATOM | 1132 | C | VAL | A | 145 | −2.874 | −10.979 | 24.546 | 1.00 | 39.72 | A |
| ATOM | 1133 | O | VAL | A | 145 | −3.824 | −11.754 | 24.513 | 1.00 | 42.40 | A |
| ATOM | 1134 | N | TYR | A | 146 | −3.010 | −9.665 | 24.386 | 1.00 | 48.62 | A |
| ATOM | 1135 | CA | TYR | A | 146 | −4.310 | −9.056 | 24.152 | 1.00 | 46.83 | A |
| ATOM | 1136 | CB | TYR | A | 146 | −4.995 | −8.703 | 25.479 | 1.00 | 42.62 | A |
| ATOM | 1137 | CG | TYR | A | 146 | −4.125 | −7.951 | 26.456 | 1.00 | 42.65 | A |
| ATOM | 1138 | CD1 | TYR | A | 146 | −3.255 | −8.629 | 27.309 | 1.00 | 42.04 | A |
| ATOM | 1139 | CE1 | TYR | A | 146 | −2.407 | −7.938 | 28.180 | 1.00 | 40.83 | A |
| ATOM | 1140 | CD2 | TYR | A | 146 | −4.134 | −6.556 | 26.498 | 1.00 | 38.24 | A |
| ATOM | 1141 | CE2 | TYR | A | 146 | −3.292 | −5.849 | 27.363 | 1.00 | 40.29 | A |
| ATOM | 1142 | CZ | TYR | A | 146 | −2.426 | −6.546 | 28.205 | 1.00 | 40.33 | A |
| ATOM | 1143 | OH | TYR | A | 146 | −1.575 | −5.870 | 29.065 | 1.00 | 41.31 | A |
| ATOM | 1144 | C | TYR | A | 146 | −4.223 | −7.816 | 23.269 | 1.00 | 45.14 | A |
| ATOM | 1145 | O | TYR | A | 146 | −3.148 | −7.255 | 23.041 | 1.00 | 43.31 | A |
| ATOM | 1146 | N | PHE | A | 147 | −5.385 | −7.387 | 22.797 | 1.00 | 40.60 | A |
| ATOM | 1147 | CA | PHE | A | 147 | −5.513 | −6.260 | 21.895 | 1.00 | 40.12 | A |
| ATOM | 1148 | CB | PHE | A | 147 | −5.324 | −6.785 | 20.471 | 1.00 | 45.79 | A |
| ATOM | 1149 | CG | PHE | A | 147 | −5.648 | −5.799 | 19.384 | 1.00 | 44.84 | A |
| ATOM | 1150 | CD1 | PHE | A | 147 | −5.364 | −4.445 | 19.526 | 1.00 | 45.33 | A |
| ATOM | 1151 | CD2 | PHE | A | 147 | −6.188 | −6.248 | 18.176 | 1.00 | 47.17 | A |
| ATOM | 1152 | CE1 | PHE | A | 147 | −5.613 | −3.554 | 18.479 | 1.00 | 45.66 | A |
| ATOM | 1153 | CE2 | PHE | A | 147 | −6.439 | −5.366 | 17.127 | 1.00 | 45.74 | A |
| ATOM | 1154 | CZ | PHE | A | 147 | −6.150 | −4.018 | 17.279 | 1.00 | 46.58 | A |
| ATOM | 1155 | C | PHE | A | 147 | −6.919 | −5.715 | 22.109 | 1.00 | 41.66 | A |
| ATOM | 1156 | O | PHE | A | 147 | −7.890 | −6.461 | 22.051 | 1.00 | 39.14 | A |
| ATOM | 1157 | N | GLY | A | 148 | −7.029 | −4.417 | 22.378 | 1.00 | 30.16 | A |
| ATOM | 1158 | CA | GLY | A | 148 | −8.333 | −3.832 | 22.609 | 1.00 | 34.64 | A |
| ATOM | 1159 | C | GLY | A | 148 | −8.434 | −2.350 | 22.308 | 1.00 | 36.86 | A |
| ATOM | 1160 | O | GLY | A | 148 | −7.439 | −1.670 | 22.070 | 1.00 | 39.47 | A |
| ATOM | 1161 | N | ILE | A | 149 | −9.666 | −1.859 | 22.301 | 1.00 | 50.38 | A |
| ATOM | 1162 | CA | ILE | A | 149 | −9.942 | −0.460 | 22.054 | 1.00 | 48.79 | A |
| ATOM | 1163 | CB | ILE | A | 149 | −10.090 | −0.140 | 20.552 | 1.00 | 58.12 | A |
| ATOM | 1164 | CG2 | ILE | A | 149 | −8.908 | −0.683 | 19.770 | 1.00 | 58.67 | A |
| ATOM | 1165 | CG1 | ILE | A | 149 | −11.368 | −0.757 | 20.005 | 1.00 | 54.16 | A |
| ATOM | 1166 | CD1 | ILE | A | 149 | −11.642 | −0.329 | 18.594 | 1.00 | 57.41 | A |
| ATOM | 1167 | C | ILE | A | 149 | −11.242 | −0.101 | 22.749 | 1.00 | 50.83 | A |
| ATOM | 1168 | O | ILE | A | 149 | −12.148 | −0.927 | 22.875 | 1.00 | 56.00 | A |
| ATOM | 1169 | N | ILE | A | 150 | −11.324 | 1.141 | 23.199 | 1.00 | 38.72 | A |
| ATOM | 1170 | CA | ILE | A | 150 | −12.503 | 1.642 | 23.884 | 1.00 | 39.23 | A |
| ATOM | 1171 | CB | ILE | A | 150 | −12.268 | 1.652 | 25.423 | 1.00 | 43.84 | A |
| ATOM | 1172 | CG2 | ILE | A | 150 | −11.007 | 2.412 | 25.753 | 1.00 | 47.14 | A |
| ATOM | 1173 | CG1 | ILE | A | 150 | −13.453 | 2.281 | 26.151 | 1.00 | 41.83 | A |
| ATOM | 1174 | CD1 | ILE | A | 150 | −14.672 | 1.425 | 26.166 | 1.00 | 39.84 | A |
| ATOM | 1175 | C | ILE | A | 150 | −12.738 | 3.062 | 23.366 | 1.00 | 40.57 | A |
| ATOM | 1176 | O | ILE | A | 150 | −11.803 | 3.840 | 23.213 | 1.00 | 39.68 | A |
| ATOM | 1177 | N | ALA | A | 151 | −13.983 | 3.391 | 23.068 | 1.00 | 48.22 | A |
| ATOM | 1178 | CA | ALA | A | 151 | −14.301 | 4.722 | 22.579 | 1.00 | 46.96 | A |
| ATOM | 1179 | CB | ALA | A | 151 | −15.619 | 4.693 | 21.829 | 1.00 | 21.38 | A |
| ATOM | 1180 | C | ALA | A | 151 | −14.384 | 5.701 | 23.750 | 1.00 | 47.92 | A |
| ATOM | 1181 | O | ALA | A | 151 | −15.133 | 5.490 | 24.704 | 1.00 | 50.30 | A |
| ATOM | 1182 | N | LEU | A | 152 | −13.609 | 6.773 | 23.683 | 1.00 | 79.22 | A |
| ATOM | 1183 | CA | LEU | A | 152 | −13.617 | 7.762 | 24.745 | 1.00 | 83.20 | A |
| ATOM | 1184 | CB | LEU | A | 152 | −12.190 | 8.134 | 25.120 | 1.00 | 51.00 | A |
| ATOM | 1185 | CG | LEU | A | 152 | −11.364 | 6.972 | 25.648 | 1.00 | 52.25 | A |
| ATOM | 1186 | CD1 | LEU | A | 152 | −9.960 | 7.442 | 25.932 | 1.00 | 49.94 | A |
| ATOM | 1187 | CD2 | LEU | A | 152 | −11.996 | 6.424 | 26.897 | 1.00 | 51.45 | A |
| ATOM | 1188 | C | LEU | A | 152 | −14.376 | 9.004 | 24.319 | 1.00 | 86.05 | A |
| ATOM | 1189 | O | LEU | A | 152 | −15.459 | 9.276 | 24.829 | 1.00 | 91.91 | A |
| ATOM | 1190 | N | ILE | A | 153 | −16.557 | −27.053 | 20.409 | 1.00 | 49.14 | A |
| ATOM | 1191 | CA | ILE | A | 153 | −16.360 | −27.912 | 21.567 | 1.00 | 49.14 | A |
| ATOM | 1192 | CB | ILE | A | 153 | −15.011 | −27.653 | 22.245 | 1.00 | 44.26 | A |
| ATOM | 1193 | CG2 | ILE | A | 153 | −13.889 | −28.120 | 21.346 | 1.00 | 41.04 | A |
| ATOM | 1194 | CG1 | ILE | A | 153 | −14.869 | −26.171 | 22.579 | 1.00 | 41.04 | A |
| ATOM | 1195 | CD1 | ILE | A | 153 | −13.557 | −25.831 | 23.273 | 1.00 | 41.04 | A |
| ATOM | 1196 | C | ILE | A | 153 | −17.461 | −27.747 | 22.602 | 1.00 | 49.14 | A |
| ATOM | 1197 | O | ILE | A | 153 | −18.145 | −26.719 | 22.663 | 1.00 | 49.14 | A |
| ATOM | 1198 | N | THR | A | 154 | −17.606 | −28.776 | 23.430 | 1.00 | 51.13 | A |
| ATOM | 1199 | CA | THR | A | 154 | −18.647 | −28.813 | 24.436 | 1.00 | 51.13 | A |
| ATOM | 1200 | CB | THR | A | 154 | −19.852 | −29.594 | 23.872 | 1.00 | 37.42 | A |
| ATOM | 1201 | OG1 | THR | A | 154 | −20.865 | −28.662 | 23.499 | 1.00 | 47.70 | A |
| ATOM | 1202 | CG2 | THR | A | 154 | −20.405 | −30.608 | 24.881 | 1.00 | 47.70 | A |
| ATOM | 1203 | C | THR | A | 154 | −18.233 | −29.432 | 25.756 | 1.00 | 51.13 | A |
| ATOM | 1204 | O | THR | A | 154 | −17.443 | −30.374 | 25.789 | 1.00 | 51.13 | A |
| ATOM | 1205 | N | LEU | A | 155 | −18.761 | −28.881 | 26.844 | 1.00 | 34.35 | A |
| ATOM | 1206 | CA | LEU | A | 155 | −18.521 | −29.438 | 28.173 | 1.00 | 34.35 | A |
| ATOM | 1207 | CB | LEU | A | 155 | −17.575 | −28.558 | 28.990 | 1.00 | 40.53 | A |
| ATOM | 1208 | CG | LEU | A | 155 | −17.198 | −29.170 | 30.358 | 1.00 | 37.11 | A |
| ATOM | 1209 | CD1 | LEU | A | 155 | −16.612 | −30.562 | 30.189 | 1.00 | 37.11 | A |
| ATOM | 1210 | CD2 | LEU | A | 155 | −16.180 | −28.287 | 31.054 | 1.00 | 37.11 | A |
| ATOM | 1211 | C | LEU | A | 155 | −19.889 | −29.562 | 28.876 | 1.00 | 34.35 | A |
| ATOM | 1212 | O | LEU | A | 155 | −20.557 | −28.559 | 29.127 | 1.00 | 34.35 | A |
| ATOM | 1213 | N | LYS | A | 156 | −20.316 | −30.787 | 29.172 | 1.00 | 41.95 | A |
| ATOM | 1214 | CA | LYS | A | 156 | −21.614 | −30.986 | 29.812 | 1.00 | 41.95 | A |
| ATOM | 1215 | CB | LYS | A | 156 | −22.576 | −31.674 | 28.832 | 1.00 | 38.22 | A |
| ATOM | 1216 | CG | LYS | A | 156 | −23.979 | −31.822 | 29.379 | 1.00 | 40.41 | A |
| ATOM | 1217 | CD | LYS | A | 156 | −24.940 | −32.481 | 28.397 | 1.00 | 40.41 | A |
| ATOM | 1218 | CE | LYS | A | 156 | −24.666 | −33.963 | 28.205 | 1.00 | 40.41 | A |
| ATOM | 1219 | NZ | LYS | A | 156 | −25.714 | −34.628 | 27.362 | 1.00 | 40.41 | A |
| ATOM | 1220 | C | LYS | A | 156 | −21.555 | −31.791 | 31.110 | 1.00 | 41.95 | A |
| ATOM | 1221 | O | LYS | A | 156 | −20.905 | −32.829 | 31.164 | 1.00 | 41.95 | A |
| ATOM | 1222 | N | TYR | A | 157 | −22.229 | −31.293 | 32.146 | 1.00 | 40.86 | A |
| ATOM | 1223 | CA | TYR | A | 157 | −22.319 | −31.954 | 33.450 | 1.00 | 40.86 | A |
| ATOM | 1224 | CB | TYR | A | 157 | −21.890 | −31.002 | 34.562 | 1.00 | 48.84 | A |
| ATOM | 1225 | CG | TYR | A | 157 | −20.395 | −30.800 | 34.680 | 1.00 | 31.18 | A |
| ATOM | 1226 | CD1 | TYR | A | 157 | −19.517 | −31.334 | 33.736 | 1.00 | 31.18 | A |
| ATOM | 1227 | CE1 | TYR | A | 157 | −18.132 | −31.134 | 33.831 | 1.00 | 31.18 | A |
| ATOM | 1228 | CD2 | TYR | A | 157 | −19.852 | −30.058 | 35.729 | 1.00 | 31.18 | A |
| ATOM | 1229 | CE2 | TYR | A | 157 | −18.472 | −29.847 | 35.831 | 1.00 | 31.18 | A |
| ATOM | 1230 | CZ | TYR | A | 157 | −17.624 | −30.386 | 34.879 | 1.00 | 31.18 | A |
| ATOM | 1231 | OH | TYR | A | 157 | −16.271 | −30.162 | 34.968 | 1.00 | 31.18 | A |
| ATOM | 1232 | C | TYR | A | 157 | −23.779 | −32.372 | 33.666 | 1.00 | 40.86 | A |
| ATOM | 1233 | O | TYR | A | 157 | −24.696 | −31.555 | 33.525 | 1.00 | 40.86 | A |
| ATOM | 1234 | N | ASN | A | 158 | −24.003 | −33.634 | 34.001 | 1.00 | 40.05 | A |
| ATOM | 1235 | CA | ASN | A | 158 | −25.357 | −34.150 | 34.193 | 1.00 | 40.05 | A |
| ATOM | 1236 | CB | ASN | A | 158 | −25.697 | −35.122 | 33.038 | 1.00 | 41.10 | A |
| ATOM | 1237 | CG | ASN | A | 158 | −27.106 | −35.709 | 33.120 | 1.00 | 51.80 | A |
| ATOM | 1238 | OD1 | ASN | A | 158 | −27.855 | −35.469 | 34.072 | 1.00 | 51.80 | A |
| ATOM | 1239 | ND2 | ASN | A | 158 | −27.447 | −36.497 | 32.098 | 1.00 | 51.80 | A |
| ATOM | 1240 | C | ASN | A | 158 | −25.448 | −34.862 | 35.535 | 1.00 | 40.05 | A |
| ATOM | 1241 | O | ASN | A | 158 | −24.784 | −35.869 | 35.749 | 1.00 | 40.05 | A |
| ATOM | 1242 | N | TYR | A | 159 | −26.241 | −34.318 | 36.447 | 1.00 | 39.38 | A |
| ATOM | 1243 | CA | TYR | A | 159 | −26.447 | −34.947 | 37.741 | 1.00 | 39.38 | A |
| ATOM | 1244 | CB | TYR | A | 159 | −26.331 | −33.946 | 38.888 | 1.00 | 34.13 | A |
| ATOM | 1245 | CG | TYR | A | 159 | −24.985 | −33.308 | 39.068 | 1.00 | 37.69 | A |
| ATOM | 1246 | CD1 | TYR | A | 159 | −24.611 | −32.205 | 38.310 | 1.00 | 37.69 | A |
| ATOM | 1247 | CE1 | TYR | A | 159 | −23.402 | −31.354 | 38.534 | 1.00 | 37.69 | A |
| ATOM | 1248 | CD2 | TYR | A | 159 | −24.110 | −33.758 | 40.051 | 1.00 | 37.69 | A |
| ATOM | 1249 | CE2 | TYR | A | 159 | −22.900 | −33.121 | 40.280 | 1.00 | 37.69 | A |
| ATOM | 1250 | CZ | TYR | A | 159 | −22.552 | −32.009 | 39.517 | 1.00 | 37.69 | A |
| ATOM | 1251 | OH | TYR | A | 159 | −21.372 | −31.328 | 39.750 | 1.00 | 37.69 | A |
| ATOM | 1252 | C | TYR | A | 159 | −27.875 | −35.481 | 37.733 | 1.00 | 39.38 | A |
| ATOM | 1253 | O | TYR | A | 159 | −28.807 | −34.739 | 37.416 | 1.00 | 39.38 | A |
| ATOM | 1254 | N | THR | A | 160 | −28.062 | −36.748 | 38.092 | 1.00 | 42.26 | A |
| ATOM | 1255 | CA | THR | A | 160 | −29.407 | −37.292 | 38.099 | 1.00 | 42.26 | A |
| ATOM | 1256 | CB | THR | A | 160 | −29.683 | −38.044 | 36.794 | 1.00 | 48.60 | A |
| ATOM | 1257 | OG1 | THR | A | 160 | −29.364 | −37.201 | 35.678 | 1.00 | 35.94 | A |
| ATOM | 1258 | CG2 | THR | A | 160 | −31.147 | −38.445 | 36.715 | 1.00 | 35.94 | A |
| ATOM | 1259 | C | THR | A | 160 | −29.753 | −38.229 | 39.251 | 1.00 | 42.26 | A |
| ATOM | 1260 | O | THR | A | 160 | −29.045 | −39.208 | 39.489 | 1.00 | 42.26 | A |
| ATOM | 1261 | N | VAL | A | 161 | −30.827 | −37.925 | 39.979 | 1.00 | 41.05 | A |
| ATOM | 1262 | CA | VAL | A | 161 | −31.278 | −38.833 | 41.028 | 1.00 | 41.05 | A |
| ATOM | 1263 | CB | VAL | A | 161 | −31.457 | −38.137 | 42.395 | 1.00 | 59.89 | A |
| ATOM | 1264 | CG1 | VAL | A | 161 | −30.231 | −37.332 | 42.710 | 1.00 | 44.40 | A |
| ATOM | 1265 | CG2 | VAL | A | 161 | −32.682 | −37.281 | 42.395 | 1.00 | 44.40 | A |
| ATOM | 1266 | C | VAL | A | 161 | −32.621 | −39.416 | 40.530 | 1.00 | 41.05 | A |
| ATOM | 1267 | O | VAL | A | 161 | −33.454 | −38.706 | 39.951 | 1.00 | 41.05 | A |
| ATOM | 1268 | N | THR | A | 162 | −32.812 | −40.714 | 40.735 | 1.00 | 71.23 | A |
| ATOM | 1269 | CA | THR | A | 162 | −34.030 | −41.384 | 40.298 | 1.00 | 71.23 | A |
| ATOM | 1270 | CB | THR | A | 162 | −33.717 | −42.364 | 39.187 | 1.00 | 84.69 | A |
| ATOM | 1271 | OG1 | THR | A | 162 | −32.661 | −41.835 | 38.381 | 1.00 | 56.02 | A |
| ATOM | 1272 | CG2 | THR | A | 162 | −34.929 | −42.570 | 38.315 | 1.00 | 56.02 | A |
| ATOM | 1273 | C | THR | A | 162 | −34.680 | −42.161 | 41.428 | 1.00 | 71.23 | A |
| ATOM | 1274 | O | THR | A | 162 | −33.985 | −42.671 | 42.309 | 1.00 | 71.23 | A |
| ATOM | 1275 | N | LEU | A | 163 | −36.006 | −42.259 | 41.410 | 1.00 | 113.54 | A |
| ATOM | 1276 | CA | LEU | A | 163 | −36.704 | −42.997 | 42.449 | 1.00 | 113.54 | A |
| ATOM | 1277 | CB | LEU | A | 163 | −38.174 | −42.600 | 42.516 | 1.00 | 125.52 | A |
| ATOM | 1278 | CG | LEU | A | 163 | −38.622 | −42.124 | 43.901 | 1.00 | 73.61 | A |
| ATOM | 1279 | CD1 | LEU | A | 163 | −40.124 | −41.947 | 43.914 | 1.00 | 73.61 | A |
| ATOM | 1280 | CD2 | LEU | A | 163 | −38.208 | −43.129 | 44.966 | 1.00 | 73.61 | A |
| ATOM | 1281 | C | LEU | A | 163 | −36.593 | −44.482 | 42.167 | 1.00 | 113.54 | A |
| ATOM | 1282 | O | LEU | A | 163 | −36.981 | −45.306 | 42.994 | 1.00 | 113.54 | A |
| ATOM | 1283 | N | LYS | A | 164 | −36.054 | −44.822 | 41.000 | 1.00 | 66.17 | A |
| ATOM | 1284 | CA | LYS | A | 164 | −35.889 | −46.223 | 40.618 | 1.00 | 66.17 | A |
| ATOM | 1285 | CB | LYS | A | 164 | −34.755 | −46.875 | 41.412 | 1.00 | 71.17 | A |
| ATOM | 1286 | CG | LYS | A | 164 | −33.462 | −47.041 | 40.638 | 1.00 | 70.92 | A |
| ATOM | 1287 | CD | LYS | A | 164 | −33.666 | −47.946 | 39.444 | 1.00 | 70.92 | A |
| ATOM | 1288 | CE | LYS | A | 164 | −32.382 | −48.151 | 38.653 | 1.00 | 70.92 | A |
| ATOM | 1289 | NZ | LYS | A | 164 | −32.558 | −49.127 | 37.521 | 1.00 | 70.92 | A |
| ATOM | 1290 | C | LYS | A | 164 | −37.188 | −46.951 | 40.906 | 1.00 | 66.17 | A |
| ATOM | 1291 | O | LYS | A | 164 | −38.226 | −46.312 | 41.066 | 1.00 | 66.17 | A |
| ATOM | 1292 | N | ASP | A | 165 | −37.146 | −48.277 | 40.967 | 1.00 | 179.88 | A |
| ATOM | 1293 | CA | ASP | A | 165 | −38.352 | −49.037 | 41.260 | 1.00 | 179.88 | A |
| ATOM | 1294 | CB | ASP | A | 165 | −38.063 | −50.540 | 41.222 | 1.00 | 145.62 | A |
| ATOM | 1295 | CG | ASP | A | 165 | −36.834 | −50.925 | 42.033 | 1.00 | 145.62 | A |
| ATOM | 1296 | OD1 | ASP | A | 165 | −36.852 | −50.765 | 43.275 | 1.00 | 145.62 | A |
| ATOM | 1297 | OD2 | ASP | A | 165 | −35.844 | −51.388 | 41.422 | 1.00 | 145.62 | A |
| ATOM | 1298 | C | ASP | A | 165 | −38.825 | −48.616 | 42.643 | 1.00 | 179.88 | A |
| ATOM | 1299 | O | ASP | A | 165 | −38.874 | −47.426 | 42.953 | 1.00 | 179.88 | A |
| ATOM | 1300 | N | ASP | A | 166 | −39.168 | −49.573 | 43.488 | 1.00 | 100.17 | A |
| ATOM | 1301 | CA | ASP | A | 166 | −39.617 | −49.189 | 44.809 | 1.00 | 100.17 | A |
| ATOM | 1302 | CB | ASP | A | 166 | −40.729 | −50.125 | 45.274 | 1.00 | 181.27 | A |
| ATOM | 1303 | CG | ASP | A | 166 | −41.979 | −49.980 | 44.433 | 1.00 | 181.27 | A |
| ATOM | 1304 | OD1 | ASP | A | 166 | −41.898 | −50.221 | 43.209 | 1.00 | 181.27 | A |
| ATOM | 1305 | OD2 | ASP | A | 166 | −43.035 | −49.613 | 44.990 | 1.00 | 181.27 | A |
| ATOM | 1306 | C | ASP | A | 166 | −38.477 | −49.148 | 45.802 | 1.00 | 100.17 | A |
| ATOM | 1307 | O | ASP | A | 166 | −37.804 | −50.163 | 46.033 | 1.00 | 100.17 | A |
| ATOM | 1308 | N | GLY | A | 167 | −38.247 | −47.958 | 46.365 | 1.00 | 151.67 | A |
| ATOM | 1309 | CA | GLY | A | 167 | −37.192 | −47.808 | 47.347 | 1.00 | 151.67 | A |
| ATOM | 1310 | C | GLY | A | 167 | −36.376 | −46.577 | 47.476 | 1.00 | 151.67 | A |
| ATOM | 1311 | O | GLY | A | 167 | −36.861 | −45.467 | 47.697 | 1.00 | 151.67 | A |
| ATOM | 1312 | N | LEU | A | 168 | −35.089 | −46.835 | 47.336 | 1.00 | 121.30 | A |
| ATOM | 1313 | CA | LEU | A | 168 | −34.063 | −45.834 | 47.440 | 1.00 | 121.30 | A |
| ATOM | 1314 | CB | LEU | A | 168 | −32.721 | −46.539 | 47.621 | 1.00 | 147.36 | A |
| ATOM | 1315 | CG | LEU | A | 168 | −32.829 | −47.874 | 48.370 | 1.00 | 147.36 | A |
| ATOM | 1316 | CD1 | LEU | A | 168 | −31.484 | −48.570 | 48.362 | 1.00 | 147.36 | A |
| ATOM | 1317 | CD2 | LEU | A | 168 | −33.319 | −47.644 | 49.799 | 1.00 | 147.36 | A |
| ATOM | 1318 | C | LEU | A | 168 | −34.031 | −44.936 | 46.210 | 1.00 | 121.30 | A |
| ATOM | 1319 | O | LEU | A | 168 | −34.729 | −45.170 | 45.223 | 1.00 | 121.30 | A |
| ATOM | 1320 | N | TYR | A | 169 | −33.209 | −43.898 | 46.295 | 1.00 | 61.11 | A |
| ATOM | 1321 | CA | TYR | A | 169 | −33.044 | −42.951 | 45.217 | 1.00 | 61.11 | A |
| ATOM | 1322 | CB | TYR | A | 169 | −33.167 | −41.524 | 45.736 | 1.00 | 66.98 | A |
| ATOM | 1323 | CG | TYR | A | 169 | −34.545 | −41.127 | 46.175 | 1.00 | 66.98 | A |
| ATOM | 1324 | CD1 | TYR | A | 169 | −35.069 | −41.557 | 47.390 | 1.00 | 66.98 | A |
| ATOM | 1325 | CE1 | TYR | A | 169 | −36.348 | −41.184 | 47.784 | 1.00 | 66.98 | A |
| ATOM | 1326 | CD2 | TYR | A | 169 | −35.331 | −40.314 | 45.367 | 1.00 | 66.98 | A |
| ATOM | 1327 | CE2 | TYR | A | 169 | −36.607 | −39.936 | 45.748 | 1.00 | 66.98 | A |
| ATOM | 1328 | CZ | TYR | A | 169 | −37.111 | −40.374 | 46.954 | 1.00 | 66.98 | A |
| ATOM | 1329 | OH | TYR | A | 169 | −38.382 | −40.016 | 47.321 | 1.00 | 66.98 | A |
| ATOM | 1330 | C | TYR | A | 169 | −31.648 | −43.151 | 44.677 | 1.00 | 61.11 | A |
| ATOM | 1331 | O | TYR | A | 169 | −30.665 | −43.038 | 45.414 | 1.00 | 61.11 | A |
| ATOM | 1332 | N | ASP | A | 170 | −31.551 | −43.456 | 43.392 | 1.00 | 38.80 | A |
| ATOM | 1333 | CA | ASP | A | 170 | −30.241 | −43.637 | 42.778 | 1.00 | 38.80 | A |
| ATOM | 1334 | CB | ASP | A | 170 | −30.296 | −44.607 | 41.609 | 1.00 | 92.31 | A |
| ATOM | 1335 | CG | ASP | A | 170 | −29.999 | −46.026 | 42.027 | 1.00 | 92.31 | A |
| ATOM | 1336 | OD1 | ASP | A | 170 | −29.126 | −46.212 | 42.907 | 1.00 | 92.31 | A |
| ATOM | 1337 | OD2 | ASP | A | 170 | −30.626 | −46.952 | 41.469 | 1.00 | 92.31 | A |
| ATOM | 1338 | C | ASP | A | 170 | −29.673 | −42.324 | 42.295 | 1.00 | 38.80 | A |
| ATOM | 1339 | O | ASP | A | 170 | −30.410 | −41.401 | 41.990 | 1.00 | 38.80 | A |
| ATOM | 1340 | N | GLY | A | 171 | −28.353 | −42.239 | 42.229 | 1.00 | 41.91 | A |
| ATOM | 1341 | CA | GLY | A | 171 | −27.731 | −41.013 | 41.781 | 1.00 | 41.91 | A |
| ATOM | 1342 | C | GLY | A | 171 | −26.556 | −41.274 | 40.857 | 1.00 | 41.91 | A |
| ATOM | 1343 | O | GLY | A | 171 | −25.732 | −42.149 | 41.090 | 1.00 | 41.91 | A |
| ATOM | 1344 | N | VAL | A | 172 | −26.490 | −40.516 | 39.779 | 1.00 | 41.38 | A |
| ATOM | 1345 | CA | VAL | A | 172 | −25.393 | −40.633 | 38.835 | 1.00 | 41.38 | A |
| ATOM | 1346 | CB | VAL | A | 172 | −25.754 | −41.408 | 37.558 | 1.00 | 60.65 | A |
| ATOM | 1347 | CG1 | VAL | A | 172 | −25.589 | −42.883 | 37.789 | 1.00 | 52.66 | A |
| ATOM | 1348 | CG2 | VAL | A | 172 | −27.171 | −41.074 | 37.131 | 1.00 | 52.66 | A |
| ATOM | 1349 | C | VAL | A | 172 | −24.986 | −39.262 | 38.381 | 1.00 | 41.38 | A |
| ATOM | 1350 | O | VAL | A | 172 | −25.759 | −38.296 | 38.436 | 1.00 | 41.38 | A |
| ATOM | 1351 | N | PHE | A | 173 | −23.752 | −39.197 | 37.916 | 1.00 | 29.87 | A |
| ATOM | 1352 | CA | PHE | A | 173 | −23.192 | −37.973 | 37.403 | 1.00 | 29.87 | A |
| ATOM | 1353 | CB | PHE | A | 173 | −22.312 | −37.306 | 38.439 | 1.00 | 55.02 | A |
| ATOM | 1354 | CG | PHE | A | 173 | −21.479 | −36.218 | 37.866 | 1.00 | 36.84 | A |
| ATOM | 1355 | CD1 | PHE | A | 173 | −22.043 | −34.980 | 37.574 | 1.00 | 36.84 | A |
| ATOM | 1356 | CD2 | PHE | A | 173 | −20.143 | −36.450 | 37.535 | 1.00 | 36.84 | A |
| ATOM | 1357 | CE1 | PHE | A | 173 | −21.292 | −33.991 | 36.956 | 1.00 | 36.84 | A |
| ATOM | 1358 | CE2 | PHE | A | 173 | −19.381 | −35.472 | 36.919 | 1.00 | 36.84 | A |
| ATOM | 1359 | CZ | PHE | A | 173 | −19.956 | −34.238 | 36.626 | 1.00 | 36.84 | A |
| ATOM | 1360 | C | PHE | A | 173 | −22.344 | −38.307 | 36.188 | 1.00 | 29.87 | A |
| ATOM | 1361 | O | PHE | A | 173 | −21.588 | −39.276 | 36.192 | 1.00 | 29.87 | A |
| ATOM | 1362 | N | TYR | A | 174 | −22.473 | −37.504 | 35.141 | 1.00 | 53.99 | A |
| ATOM | 1363 | CA | TYR | A | 174 | −21.688 | −37.717 | 33.936 | 1.00 | 53.99 | A |
| ATOM | 1364 | CB | TYR | A | 174 | −22.558 | −38.194 | 32.765 | 1.00 | 60.63 | A |
| ATOM | 1365 | CG | TYR | A | 174 | −23.152 | −39.571 | 32.932 | 1.00 | 46.67 | A |
| ATOM | 1366 | CD1 | TYR | A | 174 | −24.503 | −39.734 | 33.244 | 1.00 | 46.67 | A |
| ATOM | 1367 | CE1 | TYR | A | 174 | −25.057 | −41.001 | 33.403 | 1.00 | 46.67 | A |
| ATOM | 1368 | CD2 | TYR | A | 174 | −22.365 | −40.717 | 32.786 | 1.00 | 46.67 | A |
| ATOM | 1369 | CE2 | TYR | A | 174 | −22.909 | −41.991 | 32.946 | 1.00 | 46.67 | A |
| ATOM | 1370 | CZ | TYR | A | 174 | −24.257 | −42.125 | 33.251 | 1.00 | 46.67 | A |
| ATOM | 1371 | OH | TYR | A | 174 | −24.821 | −43.379 | 33.377 | 1.00 | 46.67 | A |
| ATOM | 1372 | C | TYR | A | 174 | −21.011 | −36.425 | 33.537 | 1.00 | 53.99 | A |
| ATOM | 1373 | O | TYR | A | 174 | −21.612 | −35.359 | 33.582 | 1.00 | 53.99 | A |
| ATOM | 1374 | N | ASP | A | 175 | −19.745 | −36.538 | 33.157 | 1.00 | 51.86 | A |
| ATOM | 1375 | CA | ASP | A | 175 | −18.944 | −35.414 | 32.688 | 1.00 | 51.86 | A |
| ATOM | 1376 | CB | ASP | A | 175 | −17.659 | −35.317 | 33.499 | 1.00 | 74.21 | A |
| ATOM | 1377 | CG | ASP | A | 175 | −16.833 | −34.101 | 33.136 | 1.00 | 62.42 | A |
| ATOM | 1378 | OD1 | ASP | A | 175 | −16.876 | −33.675 | 31.968 | 1.00 | 62.42 | A |
| ATOM | 1379 | OD2 | ASP | A | 175 | −16.120 | −33.574 | 34.012 | 1.00 | 62.42 | A |
| ATOM | 1380 | C | ASP | A | 175 | −18.607 | −35.739 | 31.224 | 1.00 | 51.86 | A |
| ATOM | 1381 | O | ASP | A | 175 | −17.884 | −36.696 | 30.941 | 1.00 | 51.86 | A |
| ATOM | 1382 | N | HIS | A | 176 | −19.138 | −34.958 | 30.292 | 1.00 | 46.63 | A |
| ATOM | 1383 | CA | HIS | A | 176 | −18.880 | −35.199 | 28.873 | 1.00 | 46.63 | A |
| ATOM | 1384 | CB | HIS | A | 176 | −20.183 | −35.482 | 28.134 | 1.00 | 71.51 | A |
| ATOM | 1385 | CG | HIS | A | 176 | −20.746 | −36.839 | 28.388 | 1.00 | 40.64 | A |
| ATOM | 1386 | CD2 | HIS | A | 176 | −21.840 | −37.234 | 29.080 | 1.00 | 40.64 | A |
| ATOM | 1387 | ND1 | HIS | A | 176 | −20.196 | −37.982 | 27.854 | 1.00 | 40.64 | A |
| ATOM | 1388 | CE1 | HIS | A | 176 | −20.928 | −39.023 | 28.201 | 1.00 | 40.64 | A |
| ATOM | 1389 | NE2 | HIS | A | 176 | −21.932 | −38.596 | 28.944 | 1.00 | 40.64 | A |
| ATOM | 1390 | C | HIS | A | 176 | −18.198 | −34.034 | 28.171 | 1.00 | 46.63 | A |
| ATOM | 1391 | O | HIS | A | 176 | −18.676 | −32.893 | 28.221 | 1.00 | 46.63 | A |
| ATOM | 1392 | N | TYR | A | 177 | −17.070 | −34.316 | 27.528 | 1.00 | 29.71 | A |
| ATOM | 1393 | CA | TYR | A | 177 | −16.357 | −33.308 | 26.736 | 1.00 | 29.71 | A |
| ATOM | 1394 | CB | TYR | A | 177 | −14.858 | −33.406 | 26.936 | 1.00 | 58.42 | A |
| ATOM | 1395 | CG | TYR | A | 177 | −14.133 | −32.440 | 26.058 | 1.00 | 51.43 | A |
| ATOM | 1396 | CD1 | TYR | A | 177 | −14.222 | −31.066 | 26.282 | 1.00 | 51.43 | A |
| ATOM | 1397 | CE1 | TYR | A | 177 | −13.607 | −30.153 | 25.425 | 1.00 | 51.43 | A |
| ATOM | 1398 | CD2 | TYR | A | 177 | −13.408 | −32.882 | 24.957 | 1.00 | 51.43 | A |
| ATOM | 1399 | CE2 | TYR | A | 177 | −12.788 | −31.975 | 24.093 | 1.00 | 51.43 | A |
| ATOM | 1400 | CZ | TYR | A | 177 | −12.895 | −30.616 | 24.336 | 1.00 | 51.43 | A |
| ATOM | 1401 | OH | TYR | A | 177 | −12.293 | −29.725 | 23.483 | 1.00 | 51.43 | A |
| ATOM | 1402 | C | TYR | A | 177 | −16.709 | −33.715 | 25.302 | 1.00 | 29.71 | A |
| ATOM | 1403 | O | TYR | A | 177 | −16.291 | −34.779 | 24.846 | 1.00 | 29.71 | A |
| ATOM | 1404 | N | ASN | A | 178 | −17.475 | −32.888 | 24.590 | 1.00 | 41.37 | A |
| ATOM | 1405 | CA | ASN | A | 178 | −17.925 | −33.257 | 23.243 | 1.00 | 41.37 | A |
| ATOM | 1406 | CB | ASN | A | 178 | −16.757 | −33.580 | 22.309 | 1.00 | 60.53 | A |
| ATOM | 1407 | CG | ASN | A | 178 | −16.034 | −32.343 | 21.836 | 1.00 | 53.00 | A |
| ATOM | 1408 | OD1 | ASN | A | 178 | −16.505 | −31.225 | 22.029 | 1.00 | 53.00 | A |
| ATOM | 1409 | ND2 | ASN | A | 178 | −14.886 | −32.535 | 21.196 | 1.00 | 53.00 | A |
| ATOM | 1410 | C | ASN | A | 178 | −18.740 | −34.513 | 23.505 | 1.00 | 41.37 | A |
| ATOM | 1411 | O | ASN | A | 178 | −19.531 | −34.538 | 24.443 | 1.00 | 41.37 | A |
| ATOM | 1412 | N | ASP | A | 179 | −18.549 | −35.554 | 22.704 | 1.00 | 52.17 | A |
| ATOM | 1413 | CA | ASP | A | 179 | −19.282 | −36.793 | 22.939 | 1.00 | 52.17 | A |
| ATOM | 1414 | CB | ASP | A | 179 | −19.751 | −37.426 | 21.616 | 1.00 | 46.20 | A |
| ATOM | 1415 | CG | ASP | A | 179 | −18.616 | −37.659 | 20.623 | 1.00 | 50.15 | A |
| ATOM | 1416 | OD1 | ASP | A | 179 | −17.433 | −37.384 | 20.957 | 1.00 | 50.15 | A |
| ATOM | 1417 | OD2 | ASP | A | 179 | −18.914 | −38.127 | 19.494 | 1.00 | 50.15 | A |
| ATOM | 1418 | C | ASP | A | 179 | −18.436 | −37.791 | 23.729 | 1.00 | 52.17 | A |
| ATOM | 1419 | O | ASP | A | 179 | −18.780 | −38.965 | 23.802 | 1.00 | 52.17 | A |
| ATOM | 1420 | N | GLN | A | 180 | −17.346 | −37.317 | 24.334 | 1.00 | 47.83 | A |
| ATOM | 1421 | CA | GLN | A | 180 | −16.439 | −38.178 | 25.099 | 1.00 | 47.83 | A |
| ATOM | 1422 | CB | GLN | A | 180 | −14.994 | −37.723 | 24.910 | 1.00 | 49.30 | A |
| ATOM | 1423 | CG | GLN | A | 180 | −14.553 | −37.609 | 23.491 | 1.00 | 40.93 | A |
| ATOM | 1424 | CD | GLN | A | 180 | −14.656 | −38.915 | 22.753 | 1.00 | 40.93 | A |
| ATOM | 1425 | OE1 | GLN | A | 180 | −15.597 | −39.135 | 21.981 | 1.00 | 40.93 | A |
| ATOM | 1426 | NE2 | GLN | A | 180 | −13.686 | −39.802 | 22.984 | 1.00 | 40.93 | A |
| ATOM | 1427 | C | GLN | A | 180 | −16.702 | −38.237 | 26.611 | 1.00 | 47.83 | A |
| ATOM | 1428 | O | GLN | A | 180 | −16.590 | −37.231 | 27.314 | 1.00 | 47.83 | A |
| ATOM | 1429 | N | LEU | A | 181 | −17.023 | −39.418 | 27.127 | 1.00 | 45.38 | A |
| ATOM | 1430 | CA | LEU | A | 181 | −17.257 | −39.533 | 28.551 | 1.00 | 45.38 | A |
| ATOM | 1431 | CB | LEU | A | 181 | −17.780 | −40.925 | 28.891 | 1.00 | 47.35 | A |
| ATOM | 1432 | CG | LEU | A | 181 | −17.920 | −41.175 | 30.391 | 1.00 | 34.78 | A |
| ATOM | 1433 | CD1 | LEU | A | 181 | −18.922 | −40.177 | 30.981 | 1.00 | 34.78 | A |
| ATOM | 1434 | CD2 | LEU | A | 181 | −18.348 | −42.622 | 30.638 | 1.00 | 34.78 | A |
| ATOM | 1435 | C | LEU | A | 181 | −15.931 | −39.268 | 29.272 | 1.00 | 45.38 | A |
| ATOM | 1436 | O | LEU | A | 181 | −14.900 | −39.824 | 28.923 | 1.00 | 45.38 | A |
| ATOM | 1437 | N | VAL | A | 182 | −15.955 | −38.407 | 30.273 | 1.00 | 42.81 | A |
| ATOM | 1438 | CA | VAL | A | 182 | −14.746 | −38.101 | 31.015 | 1.00 | 42.81 | A |
| ATOM | 1439 | CB | VAL | A | 182 | −14.536 | −36.591 | 31.113 | 1.00 | 55.81 | A |
| ATOM | 1440 | CG1 | VAL | A | 182 | −13.294 | −36.288 | 31.928 | 1.00 | 34.29 | A |
| ATOM | 1441 | CG2 | VAL | A | 182 | −14.416 | −36.003 | 29.737 | 1.00 | 34.29 | A |
| ATOM | 1442 | C | VAL | A | 182 | −14.803 | −38.664 | 32.434 | 1.00 | 42.81 | A |
| ATOM | 1443 | O | VAL | A | 182 | −13.800 | −39.152 | 32.955 | 1.00 | 42.81 | A |
| ATOM | 1444 | N | THR | A | 183 | −15.978 | −38.591 | 33.056 | 1.00 | 48.68 | A |
| ATOM | 1445 | CA | THR | A | 183 | −16.154 | −39.080 | 34.412 | 1.00 | 48.68 | A |
| ATOM | 1446 | CB | THR | A | 183 | −15.894 | −37.976 | 35.423 | 1.00 | 50.65 | A |
| ATOM | 1447 | OG1 | THR | A | 183 | −14.518 | −37.615 | 35.383 | 1.00 | 37.24 | A |
| ATOM | 1448 | CG2 | THR | A | 183 | −16.239 | −38.424 | 36.811 | 1.00 | 37.24 | A |
| ATOM | 1449 | C | THR | A | 183 | −17.554 | −39.571 | 34.677 | 1.00 | 48.68 | A |
| ATOM | 1450 | O | THR | A | 183 | −18.523 | −38.958 | 34.231 | 1.00 | 48.68 | A |
| ATOM | 1451 | N | LYS | A | 184 | −17.657 | −40.683 | 35.399 | 1.00 | 41.66 | A |
| ATOM | 1452 | CA | LYS | A | 184 | −18.953 | −41.201 | 35.792 | 1.00 | 41.66 | A |
| ATOM | 1453 | CB | LYS | A | 184 | −19.290 | −42.526 | 35.129 | 1.00 | 54.57 | A |
| ATOM | 1454 | CG | LYS | A | 184 | −20.627 | −43.062 | 35.613 | 1.00 | 46.41 | A |
| ATOM | 1455 | CD | LYS | A | 184 | −20.956 | −44.422 | 35.042 | 1.00 | 46.41 | A |
| ATOM | 1456 | CE | LYS | A | 184 | −22.265 | −44.931 | 35.601 | 1.00 | 46.41 | A |
| ATOM | 1457 | NZ | LYS | A | 184 | −22.547 | −46.322 | 35.153 | 1.00 | 46.41 | A |
| ATOM | 1458 | C | LYS | A | 184 | −18.873 | −41.418 | 37.280 | 1.00 | 41.66 | A |
| ATOM | 1459 | O | LYS | A | 184 | −17.884 | −41.958 | 37.784 | 1.00 | 41.66 | A |
| ATOM | 1460 | N | ILE | A | 185 | −19.906 | −40.974 | 37.983 | 1.00 | 47.72 | A |
| ATOM | 1461 | CA | ILE | A | 185 | −19.974 | −41.131 | 39.423 | 1.00 | 47.72 | A |
| ATOM | 1462 | CB | ILE | A | 185 | −19.677 | −39.802 | 40.141 | 1.00 | 58.21 | A |
| ATOM | 1463 | CG2 | ILE | A | 185 | −20.036 | −39.919 | 41.637 | 1.00 | 27.13 | A |
| ATOM | 1464 | CG1 | ILE | A | 185 | −18.203 | −39.429 | 39.950 | 1.00 | 27.13 | A |
| ATOM | 1465 | CD1 | ILE | A | 185 | −17.864 | −38.032 | 40.457 | 1.00 | 27.13 | A |
| ATOM | 1466 | C | ILE | A | 185 | −21.370 | −41.602 | 39.792 | 1.00 | 47.72 | A |
| ATOM | 1467 | O | ILE | A | 185 | −22.359 | −41.152 | 39.212 | 1.00 | 47.72 | A |
| ATOM | 1468 | N | SER | A | 186 | −21.455 | −42.530 | 40.737 | 1.00 | 47.36 | A |
| ATOM | 1469 | CA | SER | A | 186 | −22.753 | −43.027 | 41.175 | 1.00 | 47.36 | A |
| ATOM | 1470 | CB | SER | A | 186 | −23.070 | −44.358 | 40.505 | 1.00 | 75.66 | A |
| ATOM | 1471 | OG | SER | A | 186 | −21.949 | −45.203 | 40.547 | 1.00 | 64.77 | A |
| ATOM | 1472 | C | SER | A | 186 | −22.787 | −43.151 | 42.689 | 1.00 | 47.36 | A |
| ATOM | 1473 | O | SER | A | 186 | −21.753 | −43.337 | 43.334 | 1.00 | 47.36 | A |
| ATOM | 1474 | N | TYR | A | 187 | −23.984 | −43.011 | 43.247 | 1.00 | 48.69 | A |
| ATOM | 1475 | CA | TYR | A | 187 | −24.179 | −43.067 | 44.680 | 1.00 | 48.69 | A |
| ATOM | 1476 | CB | TYR | A | 187 | −25.521 | −42.448 | 45.036 | 1.00 | 93.67 | A |
| ATOM | 1477 | CG | TYR | A | 187 | −25.736 | −42.337 | 46.511 | 1.00 | 47.88 | A |
| ATOM | 1478 | CD1 | TYR | A | 187 | −25.018 | −41.420 | 47.267 | 1.00 | 47.88 | A |
| ATOM | 1479 | CE1 | TYR | A | 187 | −25.219 | −41.311 | 48.640 | 1.00 | 47.88 | A |
| ATOM | 1480 | CD2 | TYR | A | 187 | −26.664 | −43.152 | 47.164 | 1.00 | 47.88 | A |
| ATOM | 1481 | CE2 | TYR | A | 187 | −26.876 | −43.049 | 48.543 | 1.00 | 47.88 | A |
| ATOM | 1482 | CZ | TYR | A | 187 | −26.152 | −42.127 | 49.271 | 1.00 | 47.88 | A |
| ATOM | 1483 | OH | TYR | A | 187 | −26.359 | −42.014 | 50.628 | 1.00 | 47.88 | A |
| ATOM | 1484 | C | TYR | A | 187 | −24.129 | −44.493 | 45.187 | 1.00 | 48.69 | A |
| ATOM | 1485 | O | TYR | A | 187 | −24.918 | −45.324 | 44.759 | 1.00 | 48.69 | A |
| ATOM | 1486 | N | ASN | A | 188 | −23.190 | −44.768 | 46.092 | 1.00 | 46.73 | A |
| ATOM | 1487 | CA | ASN | A | 188 | −23.019 | −46.085 | 46.692 | 1.00 | 46.73 | A |
| ATOM | 1488 | CB | ASN | A | 188 | −21.579 | −46.233 | 47.140 | 1.00 | 182.97 | A |
| ATOM | 1489 | CG | ASN | A | 188 | −21.313 | −47.566 | 47.734 | 1.00 | 44.00 | A |
| ATOM | 1490 | OD1 | ASN | A | 188 | −22.009 | −47.996 | 48.642 | 1.00 | 44.00 | A |
| ATOM | 1491 | ND2 | ASN | A | 188 | −20.296 | −48.239 | 47.232 | 1.00 | 44.00 | A |
| ATOM | 1492 | C | ASN | A | 188 | −23.949 | −46.212 | 47.908 | 1.00 | 46.73 | A |
| ATOM | 1493 | O | ASN | A | 188 | −23.744 | −45.536 | 48.923 | 1.00 | 46.73 | A |
| ATOM | 1494 | N | HIS | A | 189 | −24.959 | −47.073 | 47.821 | 1.00 | 62.84 | A |
| ATOM | 1495 | CA | HIS | A | 189 | −25.911 | −47.240 | 48.920 | 1.00 | 62.84 | A |
| ATOM | 1496 | CB | HIS | A | 189 | −27.110 | −48.020 | 48.435 | 1.00 | 109.85 | A |
| ATOM | 1497 | CG | HIS | A | 189 | −27.984 | −47.233 | 47.520 | 1.00 | 70.39 | A |
| ATOM | 1498 | CD2 | HIS | A | 189 | −28.067 | −47.210 | 46.170 | 1.00 | 70.39 | A |
| ATOM | 1499 | ND1 | HIS | A | 189 | −28.860 | −46.271 | 47.979 | 1.00 | 70.39 | A |
| ATOM | 1500 | CE1 | HIS | A | 189 | −29.445 | −45.688 | 46.948 | 1.00 | 70.39 | A |
| ATOM | 1501 | NE2 | HIS | A | 189 | −28.980 | −46.240 | 45.839 | 1.00 | 70.39 | A |
| ATOM | 1502 | C | HIS | A | 189 | −25.369 | −47.866 | 50.190 | 1.00 | 62.84 | A |
| ATOM | 1503 | O | HIS | A | 189 | −25.869 | −47.608 | 51.290 | 1.00 | 62.84 | A |
| ATOM | 1504 | N | GLU | A | 190 | −24.347 | −48.694 | 50.037 | 1.00 | 41.73 | A |
| ATOM | 1505 | CA | GLU | A | 190 | −23.714 | −49.325 | 51.173 | 1.00 | 41.73 | A |
| ATOM | 1506 | CB | GLU | A | 190 | −22.754 | −50.397 | 50.674 | 1.00 | 103.04 | A |
| ATOM | 1507 | CG | GLU | A | 190 | −22.451 | −51.497 | 51.664 | 1.00 | 77.46 | A |
| ATOM | 1508 | CD | GLU | A | 190 | −21.579 | −52.585 | 51.062 | 1.00 | 77.46 | A |
| ATOM | 1509 | OE1 | GLU | A | 190 | −20.409 | −52.295 | 50.726 | 1.00 | 77.46 | A |
| ATOM | 1510 | OE2 | GLU | A | 190 | −22.063 | −53.729 | 50.912 | 1.00 | 77.46 | A |
| ATOM | 1511 | C | GLU | A | 190 | −22.952 | −48.231 | 51.929 | 1.00 | 41.73 | A |
| ATOM | 1512 | O | GLU | A | 190 | −23.220 | −48.001 | 53.096 | 1.00 | 41.73 | A |
| ATOM | 1513 | N | THR | A | 191 | −22.039 | −47.548 | 51.235 | 1.00 | 42.61 | A |
| ATOM | 1514 | CA | THR | A | 191 | −21.205 | −46.503 | 51.805 | 1.00 | 42.61 | A |
| ATOM | 1515 | CB | THR | A | 191 | −20.031 | −46.224 | 50.876 | 1.00 | 75.01 | A |
| ATOM | 1516 | OG1 | THR | A | 191 | −19.229 | −47.398 | 50.781 | 1.00 | 75.01 | A |
| ATOM | 1517 | CG2 | THR | A | 191 | −19.190 | −45.099 | 51.394 | 1.00 | 75.01 | A |
| ATOM | 1518 | C | THR | A | 191 | −21.910 | −45.184 | 52.104 | 1.00 | 42.61 | A |
| ATOM | 1519 | O | THR | A | 191 | −21.530 | −44.475 | 53.041 | 1.00 | 42.61 | A |
| ATOM | 1520 | N | ARG | A | 192 | −22.921 | −44.849 | 51.304 | 1.00 | 47.54 | A |
| ATOM | 1521 | CA | ARG | A | 192 | −23.679 | −43.609 | 51.473 | 1.00 | 47.54 | A |
| ATOM | 1522 | CB | ARG | A | 192 | −24.033 | −43.409 | 52.945 | 1.00 | 68.07 | A |
| ATOM | 1523 | CG | ARG | A | 192 | −24.983 | −44.451 | 53.456 | 1.00 | 68.07 | A |
| ATOM | 1524 | CD | ARG | A | 192 | −26.266 | −44.442 | 52.635 | 1.00 | 68.07 | A |
| ATOM | 1525 | NE | ARG | A | 192 | −27.158 | −45.544 | 52.992 | 1.00 | 68.07 | A |
| ATOM | 1526 | CZ | ARG | A | 192 | −27.756 | −45.695 | 54.176 | 1.00 | 68.07 | A |
| ATOM | 1527 | NH1 | ARG | A | 192 | −27.574 | −44.811 | 55.147 | 1.00 | 68.07 | A |
| ATOM | 1528 | NH2 | ARG | A | 192 | −28.536 | −46.743 | 54.403 | 1.00 | 68.07 | A |
| ATOM | 1529 | C | ARG | A | 192 | −22.986 | −42.357 | 50.932 | 1.00 | 47.54 | A |
| ATOM | 1530 | O | ARG | A | 192 | −23.181 | −41.257 | 51.443 | 1.00 | 47.54 | A |
| ATOM | 1531 | N | HIS | A | 193 | −22.178 | −42.530 | 49.895 | 1.00 | 39.61 | A |
| ATOM | 1532 | CA | HIS | A | 193 | −21.484 | −41.415 | 49.260 | 1.00 | 39.61 | A |
| ATOM | 1533 | CB | HIS | A | 193 | −20.209 | −41.039 | 50.047 | 1.00 | 66.26 | A |
| ATOM | 1534 | CG | HIS | A | 193 | −19.079 | −42.012 | 49.919 | 1.00 | 66.26 | A |
| ATOM | 1535 | CD2 | HIS | A | 193 | −18.823 | −42.978 | 49.009 | 1.00 | 66.26 | A |
| ATOM | 1536 | ND1 | HIS | A | 193 | −18.005 | −42.005 | 50.776 | 1.00 | 66.26 | A |
| ATOM | 1537 | CE1 | HIS | A | 193 | −17.133 | −42.923 | 50.402 | 1.00 | 66.26 | A |
| ATOM | 1538 | NE2 | HIS | A | 193 | −17.607 | −43.529 | 49.330 | 1.00 | 66.26 | A |
| ATOM | 1539 | C | HIS | A | 193 | −21.180 | −41.845 | 47.815 | 1.00 | 39.61 | A |
| ATOM | 1540 | O | HIS | A | 193 | −21.369 | −43.012 | 47.464 | 1.00 | 39.61 | A |
| ATOM | 1541 | N | GLY | A | 194 | −20.727 | −40.925 | 46.968 | 1.00 | 46.08 | A |
| ATOM | 1542 | CA | GLY | A | 194 | −20.452 | −41.294 | 45.590 | 1.00 | 46.08 | A |
| ATOM | 1543 | C | GLY | A | 194 | −19.124 | −41.989 | 45.355 | 1.00 | 46.08 | A |
| ATOM | 1544 | O | GLY | A | 194 | −18.233 | −41.933 | 46.195 | 1.00 | 46.08 | A |
| ATOM | 1545 | N | ASN | A | 195 | −18.998 | −42.657 | 44.209 | 1.00 | 47.09 | A |
| ATOM | 1546 | CA | ASN | A | 195 | −17.765 | −43.355 | 43.819 | 1.00 | 47.09 | A |
| ATOM | 1547 | CB | ASN | A | 195 | −17.880 | −44.863 | 43.985 | 1.00 | 57.56 | A |
| ATOM | 1548 | CG | ASN | A | 195 | −17.948 | −45.287 | 45.423 | 1.00 | 57.56 | A |
| ATOM | 1549 | OD1 | ASN | A | 195 | −17.197 | −44.791 | 46.274 | 1.00 | 57.56 | A |
| ATOM | 1550 | ND2 | ASN | A | 195 | −18.841 | −46.227 | 45.714 | 1.00 | 57.56 | A |
| ATOM | 1551 | C | ASN | A | 195 | −17.511 | −43.095 | 42.355 | 1.00 | 47.09 | A |
| ATOM | 1552 | O | ASN | A | 195 | −18.449 | −43.083 | 41.547 | 1.00 | 47.09 | A |
| ATOM | 1553 | N | VAL | A | 196 | −16.254 | −42.893 | 41.992 | 1.00 | 40.04 | A |
| ATOM | 1554 | CA | VAL | A | 196 | −15.948 | −42.660 | 40.589 | 1.00 | 40.04 | A |
| ATOM | 1555 | CB | VAL | A | 196 | −14.586 | −41.996 | 40.399 | 1.00 | 44.37 | A |
| ATOM | 1556 | CG1 | VAL | A | 196 | −14.291 | −41.861 | 38.923 | 1.00 | 44.37 | A |
| ATOM | 1557 | CG2 | VAL | A | 196 | −14.582 | −40.640 | 41.068 | 1.00 | 44.37 | A |
| ATOM | 1558 | C | VAL | A | 196 | −15.909 | −44.017 | 39.938 | 1.00 | 40.04 | A |
| ATOM | 1559 | O | VAL | A | 196 | −15.105 | −44.860 | 40.328 | 1.00 | 40.04 | A |
| ATOM | 1560 | N | ASN | A | 197 | −16.783 | −44.238 | 38.963 | 1.00 | 54.85 | A |
| ATOM | 1561 | CA | ASN | A | 197 | −16.833 | −45.524 | 38.276 | 1.00 | 54.85 | A |
| ATOM | 1562 | CB | ASN | A | 197 | −18.261 | −45.871 | 37.860 | 1.00 | 63.56 | A |
| ATOM | 1563 | CG | ASN | A | 197 | −19.252 | −45.681 | 38.972 | 1.00 | 63.56 | A |
| ATOM | 1564 | OD1 | ASN | A | 197 | −19.036 | −46.131 | 40.094 | 1.00 | 63.56 | A |
| ATOM | 1565 | ND2 | ASN | A | 197 | −20.362 | −45.018 | 38.664 | 1.00 | 63.56 | A |
| ATOM | 1566 | C | ASN | A | 197 | −15.974 | −45.487 | 37.027 | 1.00 | 54.85 | A |
| ATOM | 1567 | O | ASN | A | 197 | −15.404 | −46.489 | 36.615 | 1.00 | 54.85 | A |
| ATOM | 1568 | N | PHE | A | 198 | −15.879 | −44.318 | 36.421 | 1.00 | 44.42 | A |
| ATOM | 1569 | CA | PHE | A | 198 | −15.112 | −44.183 | 35.204 | 1.00 | 44.42 | A |
| ATOM | 1570 | CB | PHE | A | 198 | −16.050 | −44.255 | 34.006 | 1.00 | 52.73 | A |
| ATOM | 1571 | CG | PHE | A | 198 | −15.351 | −44.153 | 32.693 | 1.00 | 52.73 | A |
| ATOM | 1572 | CD1 | PHE | A | 198 | −14.923 | −45.294 | 32.034 | 1.00 | 52.73 | A |
| ATOM | 1573 | CD2 | PHE | A | 198 | −15.087 | −42.908 | 32.121 | 1.00 | 52.73 | A |
| ATOM | 1574 | CE1 | PHE | A | 198 | −14.241 | −45.200 | 30.823 | 1.00 | 52.73 | A |
| ATOM | 1575 | CE2 | PHE | A | 198 | −14.403 | −42.801 | 30.912 | 1.00 | 52.73 | A |
| ATOM | 1576 | CZ | PHE | A | 198 | −13.981 | −43.952 | 30.263 | 1.00 | 52.73 | A |
| ATOM | 1577 | C | PHE | A | 198 | −14.359 | −42.876 | 35.155 | 1.00 | 44.42 | A |
| ATOM | 1578 | O | PHE | A | 198 | −14.824 | −41.835 | 35.637 | 1.00 | 44.42 | A |
| ATOM | 1579 | N | ARG | A | 199 | −13.183 | −42.936 | 34.557 | 1.00 | 51.01 | A |
| ATOM | 1580 | CA | ARG | A | 199 | −12.357 | −41.754 | 34.370 | 1.00 | 51.01 | A |
| ATOM | 1581 | CB | ARG | A | 199 | −11.392 | −41.567 | 35.524 | 1.00 | 63.13 | A |
| ATOM | 1582 | CG | ARG | A | 199 | −10.504 | −40.367 | 35.300 | 1.00 | 63.13 | A |
| ATOM | 1583 | CD | ARG | A | 199 | −11.265 | −39.077 | 35.538 | 1.00 | 63.13 | A |
| ATOM | 1584 | NE | ARG | A | 199 | −11.034 | −38.589 | 36.897 | 1.00 | 63.13 | A |
| ATOM | 1585 | CZ | ARG | A | 199 | −11.955 | −38.011 | 37.665 | 1.00 | 63.13 | A |
| ATOM | 1586 | NH1 | ARG | A | 199 | −13.192 | −37.845 | 37.217 | 1.00 | 63.13 | A |
| ATOM | 1587 | NH2 | ARG | A | 199 | −11.635 | −37.596 | 38.889 | 1.00 | 63.13 | A |
| ATOM | 1588 | C | ARG | A | 199 | −11.559 | −41.913 | 33.086 | 1.00 | 51.01 | A |
| ATOM | 1589 | O | ARG | A | 199 | −10.749 | −42.824 | 32.977 | 1.00 | 51.01 | A |
| ATOM | 1590 | N | ALA | A | 200 | −11.791 | −41.042 | 32.111 | 1.00 | 55.84 | A |
| ATOM | 1591 | CA | ALA | A | 200 | −11.057 | −41.122 | 30.853 | 1.00 | 55.84 | A |
| ATOM | 1592 | CB | ALA | A | 200 | −11.449 | −39.984 | 29.943 | 1.00 | 51.79 | A |
| ATOM | 1593 | C | ALA | A | 200 | −9.551 | −41.088 | 31.117 | 1.00 | 55.84 | A |
| ATOM | 1594 | O | ALA | A | 200 | −9.068 | −40.301 | 31.931 | 1.00 | 55.84 | A |
| ATOM | 1595 | N | ASP | A | 201 | −8.823 | −41.963 | 30.425 | 1.00 | 61.05 | A |
| ATOM | 1596 | CA | ASP | A | 201 | −7.373 | −42.077 | 30.563 | 1.00 | 61.05 | A |
| ATOM | 1597 | CB | ASP | A | 201 | −6.870 | −43.213 | 29.669 | 1.00 | 128.23 | A |
| ATOM | 1598 | CG | ASP | A | 201 | −7.410 | −43.117 | 28.249 | 1.00 | 128.23 | A |
| ATOM | 1599 | OD1 | ASP | A | 201 | −8.638 | −43.285 | 28.064 | 1.00 | 128.23 | A |
| ATOM | 1600 | OD2 | ASP | A | 201 | −6.611 | −42.863 | 27.319 | 1.00 | 128.23 | A |
| ATOM | 1601 | C | ASP | A | 201 | −6.617 | −40.787 | 30.226 | 1.00 | 61.05 | A |
| ATOM | 1602 | O | ASP | A | 201 | −5.559 | −40.523 | 30.788 | 1.00 | 61.05 | A |
| ATOM | 1603 | N | TRP | A | 202 | −7.160 | −39.980 | 29.316 | 1.00 | 52.08 | A |
| ATOM | 1604 | CA | TRP | A | 202 | −6.497 | −38.749 | 28.906 | 1.00 | 52.08 | A |
| ATOM | 1605 | CB | TRP | A | 202 | −6.887 | −38.382 | 27.477 | 1.00 | 44.50 | A |
| ATOM | 1606 | CG | TRP | A | 202 | −8.361 | −38.288 | 27.216 | 1.00 | 44.50 | A |
| ATOM | 1607 | CD2 | TRP | A | 202 | −9.190 | −37.132 | 27.373 | 1.00 | 44.50 | A |
| ATOM | 1608 | CE2 | TRP | A | 202 | −10.501 | −37.497 | 26.971 | 1.00 | 44.50 | A |
| ATOM | 1609 | CE3 | TRP | A | 202 | −8.957 | −35.822 | 27.811 | 1.00 | 44.50 | A |
| ATOM | 1610 | CD1 | TRP | A | 202 | −9.178 | −39.281 | 26.742 | 1.00 | 44.50 | A |
| ATOM | 1611 | NE1 | TRP | A | 202 | −10.465 | −38.812 | 26.586 | 1.00 | 44.50 | A |
| ATOM | 1612 | CZ2 | TRP | A | 202 | −11.568 | −36.598 | 26.998 | 1.00 | 44.50 | A |
| ATOM | 1613 | CZ3 | TRP | A | 202 | −10.019 | −34.929 | 27.836 | 1.00 | 44.50 | A |
| ATOM | 1614 | CH2 | TRP | A | 202 | −11.306 | −35.322 | 27.433 | 1.00 | 44.50 | A |
| ATOM | 1615 | C | TRP | A | 202 | −6.727 | −37.530 | 29.784 | 1.00 | 52.08 | A |
| ATOM | 1616 | O | TRP | A | 202 | −6.070 | −36.499 | 29.608 | 1.00 | 52.08 | A |
| ATOM | 1617 | N | PHE | A | 203 | −7.641 | −37.643 | 30.736 | 1.00 | 51.47 | A |
| ATOM | 1618 | CA | PHE | A | 203 | −7.959 | −36.518 | 31.595 | 1.00 | 51.47 | A |
| ATOM | 1619 | CB | PHE | A | 203 | −9.477 | −36.444 | 31.773 | 1.00 | 48.60 | A |
| ATOM | 1620 | CG | PHE | A | 203 | −9.948 | −35.207 | 32.483 | 1.00 | 48.60 | A |
| ATOM | 1621 | CD1 | PHE | A | 203 | −9.867 | −33.959 | 31.867 | 1.00 | 48.60 | A |
| ATOM | 1622 | CD2 | PHE | A | 203 | −10.486 | −35.287 | 33.767 | 1.00 | 48.60 | A |
| ATOM | 1623 | CE1 | PHE | A | 203 | −10.317 | −32.808 | 32.519 | 1.00 | 48.60 | A |
| ATOM | 1624 | CE2 | PHE | A | 203 | −10.939 | −34.139 | 34.428 | 1.00 | 48.60 | A |
| ATOM | 1625 | CZ | PHE | A | 203 | −10.854 | −32.899 | 33.802 | 1.00 | 48.60 | A |
| ATOM | 1626 | C | PHE | A | 203 | −7.282 | −36.557 | 32.963 | 1.00 | 51.47 | A |
| ATOM | 1627 | O | PHE | A | 203 | −7.654 | −37.348 | 33.822 | 1.00 | 51.47 | A |
| ATOM | 1628 | N | ASN | A | 204 | −6.277 | −35.713 | 33.164 | 1.00 | 54.93 | A |
| ATOM | 1629 | CA | ASN | A | 204 | −5.596 | −35.636 | 34.459 | 1.00 | 54.93 | A |
| ATOM | 1630 | CB | ASN | A | 204 | −4.103 | −35.287 | 34.286 | 1.00 | 79.69 | A |
| ATOM | 1631 | CG | ASN | A | 204 | −3.422 | −34.930 | 35.609 | 1.00 | 79.69 | A |
| ATOM | 1632 | OD1 | ASN | A | 204 | −3.765 | −33.931 | 36.222 | 1.00 | 79.69 | A |
| ATOM | 1633 | ND2 | ASN | A | 204 | −2.486 | −35.756 | 36.070 | 1.00 | 79.69 | A |
| ATOM | 1634 | C | ASN | A | 204 | −6.332 | −34.513 | 35.196 | 1.00 | 54.93 | A |
| ATOM | 1635 | O | ASN | A | 204 | −6.094 | −33.328 | 34.950 | 1.00 | 54.93 | A |
| ATOM | 1636 | N | ILE | A | 205 | −7.239 | −34.884 | 36.092 | 1.00 | 57.22 | A |
| ATOM | 1637 | CA | ILE | A | 205 | −8.030 | −33.890 | 36.805 | 1.00 | 57.22 | A |
| ATOM | 1638 | CB | ILE | A | 205 | −9.017 | −34.563 | 37.781 | 1.00 | 58.57 | A |
| ATOM | 1639 | CG2 | ILE | A | 205 | −8.309 | −34.907 | 39.075 | 1.00 | 58.57 | A |
| ATOM | 1640 | CG1 | ILE | A | 205 | −10.188 | −33.619 | 38.065 | 1.00 | 58.57 | A |
| ATOM | 1641 | CD1 | ILE | A | 205 | −11.417 | −34.318 | 38.598 | 1.00 | 58.57 | A |
| ATOM | 1642 | C | ILE | A | 205 | −7.214 | −32.846 | 37.559 | 1.00 | 57.22 | A |
| ATOM | 1643 | O | ILE | A | 205 | −7.728 | −31.789 | 37.920 | 1.00 | 57.22 | A |
| ATOM | 1644 | N | SER | A | 206 | −5.943 | −33.139 | 37.800 | 1.00 | 55.64 | A |
| ATOM | 1645 | CA | SER | A | 206 | −5.077 | −32.195 | 38.505 | 1.00 | 55.64 | A |
| ATOM | 1646 | CB | SER | A | 206 | −3.809 | −32.888 | 38.996 | 1.00 | 63.18 | A |
| ATOM | 1647 | OG | SER | A | 206 | −4.114 | −33.859 | 39.979 | 1.00 | 63.18 | A |
| ATOM | 1648 | C | SER | A | 206 | −4.688 | −31.051 | 37.592 | 1.00 | 55.64 | A |
| ATOM | 1649 | O | SER | A | 206 | −4.282 | −30.003 | 38.051 | 1.00 | 55.64 | A |
| ATOM | 1650 | N | ARG | A | 207 | −4.800 | −31.261 | 36.290 | 1.00 | 57.03 | A |
| ATOM | 1651 | CA | ARG | A | 207 | −4.455 | −30.231 | 35.336 | 1.00 | 57.03 | A |
| ATOM | 1652 | CB | ARG | A | 207 | −3.821 | −30.850 | 34.088 | 1.00 | 85.18 | A |
| ATOM | 1653 | CG | ARG | A | 207 | −2.387 | −31.307 | 34.299 | 1.00 | 85.18 | A |
| ATOM | 1654 | CD | ARG | A | 207 | −1.573 | −31.200 | 33.017 | 1.00 | 85.18 | A |
| ATOM | 1655 | NE | ARG | A | 207 | −1.870 | −32.267 | 32.068 | 1.00 | 85.18 | A |
| ATOM | 1656 | CZ | ARG | A | 207 | −1.701 | −32.163 | 30.752 | 1.00 | 85.18 | A |
| ATOM | 1657 | NH1 | ARG | A | 207 | −1.242 | −31.033 | 30.232 | 1.00 | 85.18 | A |
| ATOM | 1658 | NH2 | ARG | A | 207 | −1.982 | −33.190 | 29.956 | 1.00 | 85.18 | A |
| ATOM | 1659 | C | ARG | A | 207 | −5.677 | −29.409 | 34.949 | 1.00 | 57.03 | A |
| ATOM | 1660 | O | ARG | A | 207 | −5.614 | −28.601 | 34.019 | 1.00 | 57.03 | A |
| ATOM | 1661 | N | SER | A | 208 | −6.788 | −29.608 | 35.660 | 1.00 | 48.85 | A |
| ATOM | 1662 | CA | SER | A | 208 | −8.019 | −28.871 | 35.373 | 1.00 | 48.85 | A |
| ATOM | 1663 | CB | SER | A | 208 | −9.086 | −29.790 | 34.780 | 1.00 | 64.31 | A |
| ATOM | 1664 | OG | SER | A | 208 | −10.221 | −29.034 | 34.404 | 1.00 | 64.31 | A |
| ATOM | 1665 | C | SER | A | 208 | −8.554 | −28.214 | 36.634 | 1.00 | 48.85 | A |
| ATOM | 1666 | O | SER | A | 208 | −9.582 | −28.600 | 37.177 | 1.00 | 48.85 | A |
| ATOM | 1667 | N | PRO | A | 209 | −7.848 | −27.192 | 37.108 | 1.00 | 42.21 | A |
| ATOM | 1668 | CD | PRO | A | 209 | −6.703 | −26.624 | 36.381 | 1.00 | 44.15 | A |
| ATOM | 1669 | CA | PRO | A | 209 | −8.143 | −26.394 | 38.299 | 1.00 | 42.21 | A |
| ATOM | 1670 | CB | PRO | A | 209 | −7.216 | −25.191 | 38.146 | 1.00 | 44.15 | A |
| ATOM | 1671 | CG | PRO | A | 209 | −6.062 | −25.762 | 37.433 | 1.00 | 44.15 | A |
| ATOM | 1672 | C | PRO | A | 209 | −9.589 | −25.940 | 38.434 | 1.00 | 42.21 | A |
| ATOM | 1673 | O | PRO | A | 209 | −10.065 | −25.710 | 39.538 | 1.00 | 42.21 | A |
| ATOM | 1674 | N | HIS | A | 210 | −10.285 | −25.805 | 37.312 | 1.00 | 45.71 | A |
| ATOM | 1675 | CA | HIS | A | 210 | −11.650 | −25.320 | 37.340 | 1.00 | 45.71 | A |
| ATOM | 1676 | CB | HIS | A | 210 | −11.824 | −24.274 | 36.245 | 1.00 | 57.61 | A |
| ATOM | 1677 | CG | HIS | A | 210 | −10.967 | −23.065 | 36.454 | 1.00 | 57.61 | A |
| ATOM | 1678 | CD2 | HIS | A | 210 | −9.846 | −22.645 | 35.822 | 1.00 | 57.61 | A |
| ATOM | 1679 | ND1 | HIS | A | 210 | −11.193 | −22.163 | 37.473 | 1.00 | 57.61 | A |
| ATOM | 1680 | CE1 | HIS | A | 210 | −10.248 | −21.240 | 37.458 | 1.00 | 57.61 | A |
| ATOM | 1681 | NE2 | HIS | A | 210 | −9.417 | −21.510 | 36.467 | 1.00 | 57.61 | A |
| ATOM | 1682 | C | HIS | A | 210 | −12.727 | −26.377 | 37.277 | 1.00 | 45.71 | A |
| ATOM | 1683 | O | HIS | A | 210 | −13.904 | −26.055 | 37.149 | 1.00 | 45.71 | A |
| ATOM | 1684 | N | THR | A | 211 | −12.325 | −27.641 | 37.357 | 1.00 | 49.41 | A |
| ATOM | 1685 | CA | THR | A | 211 | −13.294 | −28.724 | 37.385 | 1.00 | 49.41 | A |
| ATOM | 1686 | CB | THR | A | 211 | −12.693 | −30.046 | 36.903 | 1.00 | 54.23 | A |
| ATOM | 1687 | OG1 | THR | A | 211 | −12.303 | −29.921 | 35.530 | 1.00 | 54.23 | A |
| ATOM | 1688 | CG2 | THR | A | 211 | −13.714 | −31.161 | 37.025 | 1.00 | 54.23 | A |
| ATOM | 1689 | C | THR | A | 211 | −13.575 | −28.804 | 38.879 | 1.00 | 49.41 | A |
| ATOM | 1690 | O | THR | A | 211 | −12.647 | −28.886 | 39.670 | 1.00 | 49.41 | A |
| ATOM | 1691 | N | PRO | A | 212 | −14.851 | −28.745 | 39.286 | 1.00 | 43.27 | A |
| ATOM | 1692 | CD | PRO | A | 212 | −16.031 | −28.722 | 38.400 | 1.00 | 37.06 | A |
| ATOM | 1693 | CA | PRO | A | 212 | −15.254 | −28.807 | 40.700 | 1.00 | 43.27 | A |
| ATOM | 1694 | CB | PRO | A | 212 | −16.764 | −28.994 | 40.619 | 1.00 | 37.06 | A |
| ATOM | 1695 | CG | PRO | A | 212 | −17.128 | −28.299 | 39.338 | 1.00 | 37.06 | A |
| ATOM | 1696 | C | PRO | A | 212 | −14.587 | −29.915 | 41.528 | 1.00 | 43.27 | A |
| ATOM | 1697 | O | PRO | A | 212 | −14.401 | −31.044 | 41.049 | 1.00 | 43.27 | A |
| ATOM | 1698 | N | GLY | A | 213 | −14.241 | −29.586 | 42.771 | 1.00 | 55.43 | A |
| ATOM | 1699 | CA | GLY | A | 213 | −13.605 | −30.547 | 43.658 | 1.00 | 55.43 | A |
| ATOM | 1700 | C | GLY | A | 213 | −14.610 | −31.434 | 44.369 | 1.00 | 55.43 | A |
| ATOM | 1701 | O | GLY | A | 213 | −15.813 | −31.283 | 44.171 | 1.00 | 55.43 | A |
| ATOM | 1702 | N | ASN | A | 214 | −14.122 | −32.354 | 45.201 | 1.00 | 85.60 | A |
| ATOM | 1703 | CA | ASN | A | 214 | −14.989 | −33.288 | 45.929 | 1.00 | 85.60 | A |
| ATOM | 1704 | CB | ASN | A | 214 | −15.961 | −32.561 | 46.853 | 1.00 | 84.84 | A |
| ATOM | 1705 | CG | ASN | A | 214 | −15.336 | −31.389 | 47.546 | 1.00 | 79.01 | A |
| ATOM | 1706 | OD1 | ASN | A | 214 | −14.117 | −31.252 | 47.581 | 1.00 | 79.01 | A |
| ATOM | 1707 | ND2 | ASN | A | 214 | −16.169 | −30.531 | 48.117 | 1.00 | 79.01 | A |
| ATOM | 1708 | C | ASN | A | 214 | −15.806 | −34.070 | 44.925 | 1.00 | 85.60 | A |
| ATOM | 1709 | O | ASN | A | 214 | −17.025 | −34.164 | 45.050 | 1.00 | 85.60 | A |
| ATOM | 1710 | N | ASP | A | 215 | −15.130 | −34.625 | 43.927 | 1.00 | 68.07 | A |
| ATOM | 1711 | CA | ASP | A | 215 | −15.802 | −35.381 | 42.893 | 1.00 | 68.07 | A |
| ATOM | 1712 | CB | ASP | A | 215 | −16.218 | −36.751 | 43.419 | 1.00 | 192.65 | A |
| ATOM | 1713 | CG | ASP | A | 215 | −15.046 | −37.538 | 43.947 | 1.00 | 114.03 | A |
| ATOM | 1714 | OD1 | ASP | A | 215 | −14.072 | −37.730 | 43.188 | 1.00 | 114.03 | A |
| ATOM | 1715 | OD2 | ASP | A | 215 | −15.094 | −37.958 | 45.122 | 1.00 | 114.03 | A |
| ATOM | 1716 | C | ASP | A | 215 | −17.011 | −34.597 | 42.436 | 1.00 | 68.07 | A |
| ATOM | 1717 | O | ASP | A | 215 | −18.148 | −35.042 | 42.604 | 1.00 | 68.07 | A |
| ATOM | 1718 | N | TYR | A | 216 | −16.746 | −33.408 | 41.892 | 1.00 | 32.67 | A |
| ATOM | 1719 | CA | TYR | A | 216 | −17.792 | −32.541 | 41.355 | 1.00 | 32.67 | A |
| ATOM | 1720 | CB | TYR | A | 216 | −18.421 | −33.183 | 40.111 | 1.00 | 60.75 | A |
| ATOM | 1721 | CG | TYR | A | 216 | −17.417 | −33.603 | 39.060 | 1.00 | 39.50 | A |
| ATOM | 1722 | CD1 | TYR | A | 216 | −16.535 | −34.657 | 39.291 | 1.00 | 39.50 | A |
| ATOM | 1723 | CE1 | TYR | A | 216 | −15.599 | −35.034 | 38.344 | 1.00 | 39.50 | A |
| ATOM | 1724 | CD2 | TYR | A | 216 | −17.335 | −32.930 | 37.837 | 1.00 | 39.50 | A |
| ATOM | 1725 | CE2 | TYR | A | 216 | −16.405 | −33.294 | 36.878 | 1.00 | 39.50 | A |
| ATOM | 1726 | CZ | TYR | A | 216 | −15.537 | −34.349 | 37.136 | 1.00 | 39.50 | A |
| ATOM | 1727 | OH | TYR | A | 216 | −14.614 | −34.722 | 36.180 | 1.00 | 39.50 | A |
| ATOM | 1728 | C | TYR | A | 216 | −18.902 | −32.207 | 42.346 | 1.00 | 32.67 | A |
| ATOM | 1729 | O | TYR | A | 216 | −20.063 | −32.056 | 41.946 | 1.00 | 32.67 | A |
| ATOM | 1730 | N | ASN | A | 217 | −18.544 | −32.072 | 43.624 | 1.00 | 38.54 | A |
| ATOM | 1731 | CA | ASN | A | 217 | −19.521 | −31.774 | 44.663 | 1.00 | 38.54 | A |
| ATOM | 1732 | CB | ASN | A | 217 | −20.056 | −30.355 | 44.499 | 1.00 | 49.28 | A |
| ATOM | 1733 | CG | ASN | A | 217 | −18.978 | −29.305 | 44.630 | 1.00 | 44.01 | A |
| ATOM | 1734 | OD1 | ASN | A | 217 | −18.807 | −28.476 | 43.747 | 1.00 | 44.01 | A |
| ATOM | 1735 | ND2 | ASN | A | 217 | −18.250 | −29.326 | 45.738 | 1.00 | 44.01 | A |
| ATOM | 1736 | C | ASN | A | 217 | −20.683 | −32.758 | 44.562 | 1.00 | 38.54 | A |
| ATOM | 1737 | O | ASN | A | 217 | −21.833 | −32.379 | 44.795 | 1.00 | 38.54 | A |
| ATOM | 1738 | N | PHE | A | 218 | −20.388 | −34.013 | 44.215 | 1.00 | 36.75 | A |
| ATOM | 1739 | CA | PHE | A | 218 | −21.431 | −35.025 | 44.064 | 1.00 | 36.75 | A |
| ATOM | 1740 | CB | PHE | A | 218 | −20.849 | −36.360 | 43.620 | 1.00 | 38.71 | A |
| ATOM | 1741 | CG | PHE | A | 218 | −21.888 | −37.385 | 43.344 | 1.00 | 35.23 | A |
| ATOM | 1742 | CD1 | PHE | A | 218 | −22.546 | −37.414 | 42.129 | 1.00 | 35.23 | A |
| ATOM | 1743 | CD2 | PHE | A | 218 | −22.260 | −38.289 | 44.321 | 1.00 | 35.23 | A |
| ATOM | 1744 | CE1 | PHE | A | 218 | −23.567 | −38.335 | 41.887 | 1.00 | 35.23 | A |
| ATOM | 1745 | CE2 | PHE | A | 218 | −23.284 | −39.215 | 44.089 | 1.00 | 35.23 | A |
| ATOM | 1746 | CZ | PHE | A | 218 | −23.935 | −39.236 | 42.871 | 1.00 | 35.23 | A |
| ATOM | 1747 | C | PHE | A | 218 | −22.288 | −35.272 | 45.302 | 1.00 | 36.75 | A |
| ATOM | 1748 | O | PHE | A | 218 | −23.515 | −35.235 | 45.227 | 1.00 | 36.75 | A |
| ATOM | 1749 | N | ASN | A | 219 | −21.650 | −35.538 | 46.436 | 1.00 | 41.39 | A |
| ATOM | 1750 | CA | ASN | A | 219 | −22.404 | −35.790 | 47.651 | 1.00 | 41.39 | A |
| ATOM | 1751 | CB | ASN | A | 219 | −21.468 | −35.994 | 48.842 | 1.00 | 49.23 | A |
| ATOM | 1752 | CG | ASN | A | 219 | −20.484 | −37.122 | 48.618 | 1.00 | 40.86 | A |
| ATOM | 1753 | OD1 | ASN | A | 219 | −20.801 | −38.104 | 47.964 | 1.00 | 40.86 | A |
| ATOM | 1754 | ND2 | ASN | A | 219 | −19.288 | −36.990 | 49.169 | 1.00 | 40.86 | A |
| ATOM | 1755 | C | ASN | A | 219 | −23.337 | −34.623 | 47.910 | 1.00 | 41.39 | A |
| ATOM | 1756 | O | ASN | A | 219 | −24.499 | −34.831 | 48.248 | 1.00 | 41.39 | A |
| ATOM | 1757 | N | PHE | A | 220 | −22.843 | −33.395 | 47.737 | 1.00 | 41.47 | A |
| ATOM | 1758 | CA | PHE | A | 220 | −23.675 | −32.216 | 47.966 | 1.00 | 41.47 | A |
| ATOM | 1759 | CB | PHE | A | 220 | −22.901 | −30.938 | 47.692 | 1.00 | 46.50 | A |
| ATOM | 1760 | CG | PHE | A | 220 | −23.775 | −29.723 | 47.587 | 1.00 | 39.48 | A |
| ATOM | 1761 | CD1 | PHE | A | 220 | −24.153 | −29.024 | 48.724 | 1.00 | 39.48 | A |
| ATOM | 1762 | CD2 | PHE | A | 220 | −24.251 | −29.292 | 46.338 | 1.00 | 39.48 | A |
| ATOM | 1763 | CE1 | PHE | A | 220 | −24.987 | −27.914 | 48.640 | 1.00 | 39.48 | A |
| ATOM | 1764 | CE2 | PHE | A | 220 | −25.091 | −28.182 | 46.240 | 1.00 | 39.48 | A |
| ATOM | 1765 | CZ | PHE | A | 220 | −25.460 | −27.488 | 47.402 | 1.00 | 39.48 | A |
| ATOM | 1766 | C | PHE | A | 220 | −24.916 | −32.238 | 47.082 | 1.00 | 41.47 | A |
| ATOM | 1767 | O | PHE | A | 220 | −26.042 | −32.141 | 47.572 | 1.00 | 41.47 | A |
| ATOM | 1768 | N | TRP | A | 221 | −24.710 | −32.368 | 45.776 | 1.00 | 39.21 | A |
| ATOM | 1769 | CA | TRP | A | 221 | −25.820 | −32.383 | 44.838 | 1.00 | 39.21 | A |
| ATOM | 1770 | CB | TRP | A | 221 | −25.294 | −32.322 | 43.404 | 1.00 | 40.13 | A |
| ATOM | 1771 | CG | TRP | A | 221 | −24.756 | −30.973 | 43.040 | 1.00 | 33.28 | A |
| ATOM | 1772 | CD2 | TRP | A | 221 | −25.491 | −29.740 | 43.013 | 1.00 | 33.28 | A |
| ATOM | 1773 | CE2 | TRP | A | 221 | −24.576 | −28.716 | 42.666 | 1.00 | 33.28 | A |
| ATOM | 1774 | CE3 | TRP | A | 221 | −26.836 | −29.399 | 43.251 | 1.00 | 33.28 | A |
| ATOM | 1775 | CD1 | TRP | A | 221 | −23.465 | −30.655 | 42.714 | 1.00 | 33.28 | A |
| ATOM | 1776 | NE1 | TRP | A | 221 | −23.349 | −29.305 | 42.490 | 1.00 | 33.28 | A |
| ATOM | 1777 | CZ2 | TRP | A | 221 | −24.966 | −27.368 | 42.548 | 1.00 | 33.28 | A |
| ATOM | 1778 | CZ3 | TRP | A | 221 | −27.221 | −28.061 | 43.134 | 1.00 | 33.28 | A |
| ATOM | 1779 | CH2 | TRP | A | 221 | −26.286 | −27.064 | 42.785 | 1.00 | 33.28 | A |
| ATOM | 1780 | C | TRP | A | 221 | −26.753 | −33.578 | 45.005 | 1.00 | 39.21 | A |
| ATOM | 1781 | O | TRP | A | 221 | −27.978 | −33.435 | 45.019 | 1.00 | 39.21 | A |
| ATOM | 1782 | N | TYR | A | 222 | −26.187 | −34.767 | 45.129 | 1.00 | 44.19 | A |
| ATOM | 1783 | CA | TYR | A | 222 | −27.023 | −35.942 | 45.295 | 1.00 | 44.19 | A |
| ATOM | 1784 | CB | TYR | A | 222 | −26.180 | −37.166 | 45.628 | 1.00 | 46.77 | A |
| ATOM | 1785 | CG | TYR | A | 222 | −27.037 | −38.331 | 46.033 | 1.00 | 45.34 | A |
| ATOM | 1786 | CD1 | TYR | A | 222 | −27.699 | −39.095 | 45.073 | 1.00 | 45.34 | A |
| ATOM | 1787 | CE1 | TYR | A | 222 | −28.559 | −40.117 | 45.440 | 1.00 | 45.34 | A |
| ATOM | 1788 | CD2 | TYR | A | 222 | −27.256 | −38.621 | 47.379 | 1.00 | 45.34 | A |
| ATOM | 1789 | CE2 | TYR | A | 222 | −28.114 | −39.631 | 47.765 | 1.00 | 45.34 | A |
| ATOM | 1790 | CZ | TYR | A | 222 | −28.767 | −40.379 | 46.786 | 1.00 | 45.34 | A |
| ATOM | 1791 | OH | TYR | A | 222 | −29.648 | −41.378 | 47.133 | 1.00 | 45.34 | A |
| ATOM | 1792 | C | TYR | A | 222 | −28.017 | −35.713 | 46.426 | 1.00 | 44.19 | A |
| ATOM | 1793 | O | TYR | A | 222 | −29.226 | −35.877 | 46.268 | 1.00 | 44.19 | A |
| ATOM | 1794 | N | SER | A | 223 | −27.487 | −35.334 | 47.576 | 1.00 | 42.77 | A |
| ATOM | 1795 | CA | SER | A | 223 | −28.311 | −35.102 | 48.740 | 1.00 | 42.77 | A |
| ATOM | 1796 | CB | SER | A | 223 | −27.419 | −34.764 | 49.927 | 1.00 | 64.40 | A |
| ATOM | 1797 | OG | SER | A | 223 | −28.212 | −34.374 | 51.027 | 1.00 | 63.02 | A |
| ATOM | 1798 | C | SER | A | 223 | −29.361 | −34.004 | 48.541 | 1.00 | 42.77 | A |
| ATOM | 1799 | O | SER | A | 223 | −30.539 | −34.210 | 48.818 | 1.00 | 42.77 | A |
| ATOM | 1800 | N | LEU | A | 224 | −28.933 | −32.845 | 48.055 | 1.00 | 37.16 | A |
| ATOM | 1801 | CA | LEU | A | 224 | −29.846 | −31.737 | 47.863 | 1.00 | 37.16 | A |
| ATOM | 1802 | CB | LEU | A | 224 | −29.069 | −30.468 | 47.520 | 1.00 | 55.65 | A |
| ATOM | 1803 | CG | LEU | A | 224 | −29.947 | −29.236 | 47.326 | 1.00 | 32.89 | A |
| ATOM | 1804 | CD1 | LEU | A | 224 | −30.864 | −29.021 | 48.523 | 1.00 | 32.89 | A |
| ATOM | 1805 | CD2 | LEU | A | 224 | −29.049 | −28.047 | 47.101 | 1.00 | 32.89 | A |
| ATOM | 1806 | C | LEU | A | 224 | −30.869 | −32.036 | 46.786 | 1.00 | 37.16 | A |
| ATOM | 1807 | O | LEU | A | 224 | −31.986 | −31.549 | 46.843 | 1.00 | 37.16 | A |
| ATOM | 1808 | N | MET | A | 225 | −30.493 | −32.829 | 45.792 | 1.00 | 51.59 | A |
| ATOM | 1809 | CA | MET | A | 225 | −31.429 | −33.170 | 44.723 | 1.00 | 51.59 | A |
| ATOM | 1810 | CB | MET | A | 225 | −30.685 | −33.700 | 43.515 | 1.00 | 79.87 | A |
| ATOM | 1811 | CG | MET | A | 225 | −29.869 | −32.644 | 42.846 | 1.00 | 48.59 | A |
| ATOM | 1812 | SD | MET | A | 225 | −29.148 | −33.211 | 41.334 | 1.00 | 48.59 | A |
| ATOM | 1813 | CE | MET | A | 225 | −30.641 | −33.518 | 40.391 | 1.00 | 48.59 | A |
| ATOM | 1814 | C | MET | A | 225 | −32.409 | −34.212 | 45.226 | 1.00 | 51.59 | A |
| ATOM | 1815 | O | MET | A | 225 | −33.573 | −34.222 | 44.857 | 1.00 | 51.59 | A |
| ATOM | 1816 | N | LYS | A | 226 | −31.927 | −35.103 | 46.075 | 1.00 | 42.81 | A |
| ATOM | 1817 | CA | LYS | A | 226 | −32.783 | −36.119 | 46.649 | 1.00 | 42.81 | A |
| ATOM | 1818 | CB | LYS | A | 226 | −31.967 | −37.009 | 47.582 | 1.00 | 104.78 | A |
| ATOM | 1819 | CG | LYS | A | 226 | −32.763 | −38.116 | 48.212 | 1.00 | 56.24 | A |
| ATOM | 1820 | CD | LYS | A | 226 | −31.948 | −38.831 | 49.248 | 1.00 | 56.24 | A |
| ATOM | 1821 | CE | LYS | A | 226 | −32.807 | −39.768 | 50.077 | 1.00 | 56.24 | A |
| ATOM | 1822 | NZ | LYS | A | 226 | −31.988 | −40.472 | 51.121 | 1.00 | 56.24 | A |
| ATOM | 1823 | C | LYS | A | 226 | −33.862 | −35.362 | 47.442 | 1.00 | 42.81 | A |
| ATOM | 1824 | O | LYS | A | 226 | −35.040 | −35.662 | 47.337 | 1.00 | 42.81 | A |
| ATOM | 1825 | N | GLU | A | 227 | −33.447 | −34.371 | 48.230 | 1.00 | 57.88 | A |
| ATOM | 1826 | CA | GLU | A | 227 | −34.384 | −33.582 | 49.026 | 1.00 | 57.88 | A |
| ATOM | 1827 | CB | GLU | A | 227 | −33.644 | −32.494 | 49.811 | 1.00 | 151.02 | A |
| ATOM | 1828 | CG | GLU | A | 227 | −34.537 | −31.709 | 50.757 | 1.00 | 126.82 | A |
| ATOM | 1829 | CD | GLU | A | 227 | −33.888 | −30.434 | 51.263 | 1.00 | 126.82 | A |
| ATOM | 1830 | OE1 | GLU | A | 227 | −32.750 | −30.500 | 51.777 | 1.00 | 126.82 | A |
| ATOM | 1831 | OE2 | GLU | A | 227 | −34.522 | −29.363 | 51.153 | 1.00 | 126.82 | A |
| ATOM | 1832 | C | GLU | A | 227 | −35.407 | −32.926 | 48.102 | 1.00 | 57.88 | A |
| ATOM | 1833 | O | GLU | A | 227 | −36.601 | −32.982 | 48.350 | 1.00 | 57.88 | A |
| ATOM | 1834 | N | THR | A | 228 | −34.926 | −32.314 | 47.026 | 1.00 | 46.68 | A |
| ATOM | 1835 | CA | THR | A | 228 | −35.785 | −31.632 | 46.077 | 1.00 | 46.68 | A |
| ATOM | 1836 | CB | THR | A | 228 | −34.969 | −30.947 | 44.992 | 1.00 | 55.19 | A |
| ATOM | 1837 | OG1 | THR | A | 228 | −34.065 | −30.023 | 45.603 | 1.00 | 41.18 | A |
| ATOM | 1838 | CG2 | THR | A | 228 | −35.895 | −30.209 | 44.017 | 1.00 | 41.18 | A |
| ATOM | 1839 | C | THR | A | 228 | −36.767 | −32.567 | 45.404 | 1.00 | 46.68 | A |
| ATOM | 1840 | O | THR | A | 228 | −37.919 | −32.214 | 45.182 | 1.00 | 46.68 | A |
| ATOM | 1841 | N | LEU | A | 229 | −36.313 | −33.760 | 45.065 | 1.00 | 60.53 | A |
| ATOM | 1842 | CA | LEU | A | 229 | −37.193 | −34.708 | 44.421 | 1.00 | 60.53 | A |
| ATOM | 1843 | CB | LEU | A | 229 | −36.395 | −35.918 | 43.959 | 1.00 | 70.94 | A |
| ATOM | 1844 | CG | LEU | A | 229 | −37.186 | −36.998 | 43.215 | 1.00 | 42.82 | A |
| ATOM | 1845 | CD1 | LEU | A | 229 | −38.147 | −36.371 | 42.196 | 1.00 | 42.82 | A |
| ATOM | 1846 | CD2 | LEU | A | 229 | −36.205 | −37.946 | 42.526 | 1.00 | 42.82 | A |
| ATOM | 1847 | C | LEU | A | 229 | −38.319 | −35.131 | 45.371 | 1.00 | 60.53 | A |
| ATOM | 1848 | O | LEU | A | 229 | −39.470 | −35.251 | 44.956 | 1.00 | 60.53 | A |
| ATOM | 1849 | N | GLU | A | 230 | −37.991 | −35.344 | 46.644 | 1.00 | 65.22 | A |
| ATOM | 1850 | CA | GLU | A | 230 | −38.990 | −35.739 | 47.632 | 1.00 | 65.22 | A |
| ATOM | 1851 | CB | GLU | A | 230 | −38.325 | −35.991 | 48.990 | 1.00 | 142.89 | A |
| ATOM | 1852 | CG | GLU | A | 230 | −37.461 | −37.246 | 49.018 | 1.00 | 107.35 | A |
| ATOM | 1853 | CD | GLU | A | 230 | −36.841 | −37.512 | 50.381 | 1.00 | 107.35 | A |
| ATOM | 1854 | OE1 | GLU | A | 230 | −37.594 | −37.524 | 51.377 | 1.00 | 107.35 | A |
| ATOM | 1855 | OE2 | GLU | A | 230 | −35.608 | −37.717 | 50.456 | 1.00 | 107.35 | A |
| ATOM | 1856 | C | GLU | A | 230 | −40.073 | −34.669 | 47.764 | 1.00 | 65.22 | A |
| ATOM | 1857 | O | GLU | A | 230 | −41.259 | −34.988 | 47.751 | 1.00 | 65.22 | A |
| ATOM | 1858 | N | GLU | A | 231 | −39.665 | −33.405 | 47.879 | 1.00 | 65.15 | A |
| ATOM | 1859 | CA | GLU | A | 231 | −40.610 | −32.299 | 48.013 | 1.00 | 65.15 | A |
| ATOM | 1860 | CB | GLU | A | 231 | −39.865 | −30.972 | 48.182 | 1.00 | 142.65 | A |
| ATOM | 1861 | CG | GLU | A | 231 | −38.883 | −30.947 | 49.342 | 1.00 | 118.89 | A |
| ATOM | 1862 | CD | GLU | A | 231 | −39.561 | −31.013 | 50.697 | 1.00 | 118.89 | A |
| ATOM | 1863 | OE1 | GLU | A | 231 | −40.346 | −31.957 | 50.929 | 1.00 | 118.89 | A |
| ATOM | 1864 | OE2 | GLU | A | 231 | −39.299 | −30.119 | 51.533 | 1.00 | 118.89 | A |
| ATOM | 1865 | C | GLU | A | 231 | −41.494 | −32.233 | 46.771 | 1.00 | 65.15 | A |
| ATOM | 1866 | O | GLU | A | 231 | −42.710 | −32.078 | 46.862 | 1.00 | 65.15 | A |
| ATOM | 1867 | N | ILE | A | 232 | −40.871 | −32.355 | 45.607 | 1.00 | 81.41 | A |
| ATOM | 1868 | CA | ILE | A | 232 | −41.595 | −32.308 | 44.345 | 1.00 | 81.41 | A |
| ATOM | 1869 | CB | ILE | A | 232 | −40.637 | −32.471 | 43.149 | 1.00 | 84.86 | A |
| ATOM | 1870 | CG2 | ILE | A | 232 | −41.408 | −32.790 | 41.903 | 1.00 | 54.91 | A |
| ATOM | 1871 | CG1 | ILE | A | 232 | −39.843 | −31.183 | 42.940 | 1.00 | 54.91 | A |
| ATOM | 1872 | CD1 | ILE | A | 232 | −38.849 | −31.257 | 41.805 | 1.00 | 54.91 | A |
| ATOM | 1873 | C | ILE | A | 232 | −42.665 | −33.384 | 44.262 | 1.00 | 81.41 | A |
| ATOM | 1874 | O | ILE | A | 232 | −43.717 | −33.168 | 43.666 | 1.00 | 81.41 | A |
| ATOM | 1875 | N | ASN | A | 233 | −42.406 | −34.538 | 44.863 | 1.00 | 74.71 | A |
| ATOM | 1876 | CA | ASN | A | 233 | −43.373 | −35.623 | 44.823 | 1.00 | 74.71 | A |
| ATOM | 1877 | CB | ASN | A | 233 | −42.674 | −36.968 | 44.999 | 1.00 | 68.62 | A |
| ATOM | 1878 | CG | ASN | A | 233 | −41.834 | −37.342 | 43.802 | 1.00 | 57.54 | A |
| ATOM | 1879 | OD1 | ASN | A | 233 | −42.164 | −36.994 | 42.675 | 1.00 | 57.54 | A |
| ATOM | 1880 | ND2 | ASN | A | 233 | −40.749 | −38.070 | 44.037 | 1.00 | 57.54 | A |
| ATOM | 1881 | C | ASN | A | 233 | −44.529 | −35.527 | 45.816 | 1.00 | 74.71 | A |
| ATOM | 1882 | O | ASN | A | 233 | −45.489 | −36.291 | 45.704 | 1.00 | 74.71 | A |
| ATOM | 1883 | N | LYS | A | 234 | −44.454 | −34.612 | 46.781 | 1.00 | 80.66 | A |
| ATOM | 1884 | CA | LYS | A | 234 | −45.541 | −34.472 | 47.748 | 1.00 | 80.66 | A |
| ATOM | 1885 | CB | LYS | A | 234 | −45.131 | −33.538 | 48.885 | 1.00 | 111.56 | A |
| ATOM | 1886 | CG | LYS | A | 234 | −44.152 | −34.202 | 49.837 | 1.00 | 111.56 | A |
| ATOM | 1887 | CD | LYS | A | 234 | −43.767 | −33.313 | 51.001 | 1.00 | 111.56 | A |
| ATOM | 1888 | CE | LYS | A | 234 | −42.820 | −34.052 | 51.937 | 1.00 | 111.56 | A |
| ATOM | 1889 | NZ | LYS | A | 234 | −42.295 | −33.176 | 53.021 | 1.00 | 111.56 | A |
| ATOM | 1890 | C | LYS | A | 234 | −46.830 | −33.998 | 47.071 | 1.00 | 80.66 | A |
| ATOM | 1891 | O | LYS | A | 234 | −47.905 | −34.540 | 47.337 | 1.00 | 80.66 | A |
| ATOM | 1892 | N | ASN | A | 235 | −46.738 | −32.987 | 46.207 | 1.00 | 127.83 | A |
| ATOM | 1893 | CA | ASN | A | 235 | −47.920 | −32.539 | 45.473 | 1.00 | 127.83 | A |
| ATOM | 1894 | CB | ASN | A | 235 | −47.697 | −31.167 | 44.817 | 1.00 | 150.99 | A |
| ATOM | 1895 | CG | ASN | A | 235 | −48.867 | −30.748 | 43.925 | 1.00 | 150.99 | A |
| ATOM | 1896 | OD1 | ASN | A | 235 | −49.378 | −31.564 | 43.161 | 1.00 | 150.99 | A |
| ATOM | 1897 | ND2 | ASN | A | 235 | −49.284 | −29.485 | 44.005 | 1.00 | 150.99 | A |
| ATOM | 1898 | C | ASN | A | 235 | −48.031 | −33.618 | 44.398 | 1.00 | 127.83 | A |
| ATOM | 1899 | O | ASN | A | 235 | −47.209 | −33.676 | 43.486 | 1.00 | 127.83 | A |
| ATOM | 1900 | N | ASP | A | 236 | −49.038 | −34.477 | 44.518 | 1.00 | 133.49 | A |
| ATOM | 1901 | CA | ASP | A | 236 | −49.226 | −35.586 | 43.584 | 1.00 | 133.49 | A |
| ATOM | 1902 | CB | ASP | A | 236 | −50.411 | −36.441 | 44.039 | 1.00 | 141.39 | A |
| ATOM | 1903 | CG | ASP | A | 236 | −50.318 | −37.876 | 43.556 | 1.00 | 141.39 | A |
| ATOM | 1904 | OD1 | ASP | A | 236 | −50.489 | −38.113 | 42.342 | 1.00 | 141.39 | A |
| ATOM | 1905 | OD2 | ASP | A | 236 | −50.068 | −38.769 | 44.394 | 1.00 | 141.39 | A |
| ATOM | 1906 | C | ASP | A | 236 | −49.396 | −35.216 | 42.108 | 1.00 | 133.49 | A |
| ATOM | 1907 | O | ASP | A | 236 | −49.424 | −36.100 | 41.252 | 1.00 | 133.49 | A |
| ATOM | 1908 | N | SER | A | 237 | −49.501 | −33.925 | 41.802 | 1.00 | 116.35 | A |
| ATOM | 1909 | CA | SER | A | 237 | −49.659 | −33.496 | 40.409 | 1.00 | 116.35 | A |
| ATOM | 1910 | CB | SER | A | 237 | −50.584 | −32.274 | 40.311 | 1.00 | 86.54 | A |
| ATOM | 1911 | OG | SER | A | 237 | −49.928 | −31.084 | 40.722 | 1.00 | 86.54 | A |
| ATOM | 1912 | C | SER | A | 237 | −48.314 | −33.160 | 39.765 | 1.00 | 116.35 | A |
| ATOM | 1913 | O | SER | A | 237 | −48.258 | −32.711 | 38.620 | 1.00 | 116.35 | A |
| ATOM | 1914 | N | THR | A | 238 | −47.233 | −33.374 | 40.510 | 1.00 | 113.89 | A |
| ATOM | 1915 | CA | THR | A | 238 | −45.888 | −33.101 | 40.013 | 1.00 | 113.89 | A |
| ATOM | 1916 | CB | THR | A | 238 | −45.174 | −32.036 | 40.873 | 1.00 | 115.30 | A |
| ATOM | 1917 | OG1 | THR | A | 238 | −46.068 | −30.948 | 41.138 | 1.00 | 115.30 | A |
| ATOM | 1918 | CG2 | THR | A | 238 | −43.950 | −31.504 | 40.142 | 1.00 | 115.30 | A |
| ATOM | 1919 | C | THR | A | 238 | −45.050 | −34.377 | 40.051 | 1.00 | 113.89 | A |
| ATOM | 1920 | O | THR | A | 238 | −44.090 | −34.519 | 39.296 | 1.00 | 113.89 | A |
| ATOM | 1921 | N | LYS | A | 239 | −45.426 | −35.293 | 40.943 | 1.00 | 120.67 | A |
| ATOM | 1922 | CA | LYS | A | 239 | −44.738 | −36.571 | 41.121 | 1.00 | 120.67 | A |
| ATOM | 1923 | CB | LYS | A | 239 | −45.754 | −37.667 | 41.472 | 1.00 | 127.62 | A |
| ATOM | 1924 | CG | LYS | A | 239 | −45.138 | −39.029 | 41.755 | 1.00 | 127.62 | A |
| ATOM | 1925 | CD | LYS | A | 239 | −46.157 | −39.982 | 42.349 | 1.00 | 127.62 | A |
| ATOM | 1926 | CE | LYS | A | 239 | −46.661 | −39.478 | 43.693 | 1.00 | 127.62 | A |
| ATOM | 1927 | NZ | LYS | A | 239 | −47.665 | −40.402 | 44.286 | 1.00 | 127.62 | A |
| ATOM | 1928 | C | LYS | A | 239 | −43.937 | −36.970 | 39.885 | 1.00 | 120.67 | A |
| ATOM | 1929 | O | LYS | A | 239 | −44.482 | −37.079 | 38.789 | 1.00 | 120.67 | A |
| ATOM | 1930 | N | THR | A | 240 | −42.639 | −37.185 | 40.069 | 1.00 | 63.35 | A |
| ATOM | 1931 | CA | THR | A | 240 | −41.765 | −37.546 | 38.964 | 1.00 | 63.35 | A |
| ATOM | 1932 | CB | THR | A | 240 | −40.882 | −36.361 | 38.546 | 1.00 | 76.02 | A |
| ATOM | 1933 | OG1 | THR | A | 240 | −41.703 | −35.227 | 38.252 | 1.00 | 76.02 | A |
| ATOM | 1934 | CG2 | THR | A | 240 | −40.069 | −36.717 | 37.321 | 1.00 | 76.02 | A |
| ATOM | 1935 | C | THR | A | 240 | −40.847 | −38.689 | 39.324 | 1.00 | 63.35 | A |
| ATOM | 1936 | O | THR | A | 240 | −40.551 | −38.916 | 40.499 | 1.00 | 63.35 | A |
| ATOM | 1937 | N | THR | A | 241 | −40.386 | −39.389 | 38.294 | 1.00 | 63.92 | A |
| ATOM | 1938 | CA | THR | A | 241 | −39.483 | −40.520 | 38.443 | 1.00 | 63.92 | A |
| ATOM | 1939 | CB | THR | A | 241 | −39.334 | −41.269 | 37.136 | 1.00 | 106.81 | A |
| ATOM | 1940 | OG1 | THR | A | 241 | −40.626 | −41.523 | 36.591 | 1.00 | 66.51 | A |
| ATOM | 1941 | CG2 | THR | A | 241 | −38.601 | −42.575 | 37.356 | 1.00 | 66.51 | A |
| ATOM | 1942 | C | THR | A | 241 | −38.085 | −40.071 | 38.834 | 1.00 | 63.92 | A |
| ATOM | 1943 | O | THR | A | 241 | −37.436 | −40.686 | 39.692 | 1.00 | 63.92 | A |
| ATOM | 1944 | N | SER | A | 242 | −37.622 | −38.999 | 38.192 | 1.00 | 69.96 | A |
| ATOM | 1945 | CA | SER | A | 242 | −36.285 | −38.478 | 38.447 | 1.00 | 69.96 | A |
| ATOM | 1946 | CB | SER | A | 242 | −35.291 | −39.163 | 37.520 | 1.00 | 76.43 | A |
| ATOM | 1947 | OG | SER | A | 242 | −35.597 | −38.849 | 36.174 | 1.00 | 48.95 | A |
| ATOM | 1948 | C | SER | A | 242 | −36.140 | −36.970 | 38.280 | 1.00 | 69.96 | A |
| ATOM | 1949 | O | SER | A | 242 | −37.038 | −36.290 | 37.786 | 1.00 | 69.96 | A |
| ATOM | 1950 | N | LEU | A | 243 | −34.971 | −36.476 | 38.683 | 1.00 | 42.66 | A |
| ATOM | 1951 | CA | LEU | A | 243 | −34.624 | −35.064 | 38.627 | 1.00 | 42.66 | A |
| ATOM | 1952 | CB | LEU | A | 243 | −34.701 | −34.464 | 40.027 | 1.00 | 67.62 | A |
| ATOM | 1953 | CG | LEU | A | 243 | −34.657 | −32.945 | 40.107 | 1.00 | 43.75 | A |
| ATOM | 1954 | CD1 | LEU | A | 243 | −35.799 | −32.368 | 39.287 | 1.00 | 43.75 | A |
| ATOM | 1955 | CD2 | LEU | A | 243 | −34.788 | −32.504 | 41.560 | 1.00 | 43.75 | A |
| ATOM | 1956 | C | LEU | A | 243 | −33.203 | −34.955 | 38.095 | 1.00 | 42.66 | A |
| ATOM | 1957 | O | LEU | A | 243 | −32.338 | −35.754 | 38.445 | 1.00 | 42.66 | A |
| ATOM | 1958 | N | SER | A | 244 | −32.964 | −33.980 | 37.233 | 1.00 | 38.22 | A |
| ATOM | 1959 | CA | SER | A | 244 | −31.634 | −33.790 | 36.655 | 1.00 | 38.22 | A |
| ATOM | 1960 | CB | SER | A | 244 | −31.603 | −34.252 | 35.198 | 1.00 | 28.73 | A |
| ATOM | 1961 | OG | SER | A | 244 | −31.770 | −35.645 | 35.082 | 1.00 | 30.88 | A |
| ATOM | 1962 | C | SER | A | 244 | −31.135 | −32.346 | 36.692 | 1.00 | 38.22 | A |
| ATOM | 1963 | O | SER | A | 244 | −31.900 | −31.394 | 36.477 | 1.00 | 38.22 | A |
| ATOM | 1964 | N | LEU | A | 245 | −29.844 | −32.189 | 36.967 | 1.00 | 37.10 | A |
| ATOM | 1965 | CA | LEU | A | 245 | −29.223 | −30.873 | 36.989 | 1.00 | 37.10 | A |
| ATOM | 1966 | CB | LEU | A | 245 | −28.427 | −30.665 | 38.266 | 1.00 | 53.99 | A |
| ATOM | 1967 | CG | LEU | A | 245 | −27.669 | −29.333 | 38.276 | 1.00 | 37.98 | A |
| ATOM | 1968 | CD1 | LEU | A | 245 | −28.661 | −28.200 | 38.121 | 1.00 | 37.98 | A |
| ATOM | 1969 | CD2 | LEU | A | 245 | −26.860 | −29.170 | 39.569 | 1.00 | 37.98 | A |
| ATOM | 1970 | C | LEU | A | 245 | −28.288 | −30.824 | 35.784 | 1.00 | 37.10 | A |
| ATOM | 1971 | O | LEU | A | 245 | −27.235 | −31.445 | 35.778 | 1.00 | 37.10 | A |
| ATOM | 1972 | N | ILE | A | 246 | −28.688 | −30.098 | 34.750 | 1.00 | 36.39 | A |
| ATOM | 1973 | CA | ILE | A | 246 | −27.893 | −29.998 | 33.531 | 1.00 | 36.39 | A |
| ATOM | 1974 | CB | ILE | A | 246 | −28.793 | −30.085 | 32.278 | 1.00 | 52.87 | A |
| ATOM | 1975 | CG2 | ILE | A | 246 | −27.947 | −30.080 | 31.021 | 1.00 | 35.91 | A |
| ATOM | 1976 | CG1 | ILE | A | 246 | −29.631 | −31.359 | 32.322 | 1.00 | 35.91 | A |
| ATOM | 1977 | CD1 | ILE | A | 246 | −28.821 | −32.601 | 32.478 | 1.00 | 35.91 | A |
| ATOM | 1978 | C | ILE | A | 246 | −27.158 | −28.671 | 33.503 | 1.00 | 36.39 | A |
| ATOM | 1979 | O | ILE | A | 246 | −27.769 | −27.609 | 33.501 | 1.00 | 36.39 | A |
| ATOM | 1980 | N | THR | A | 247 | −25.837 | −28.721 | 33.489 | 1.00 | 41.29 | A |
| ATOM | 1981 | CA | THR | A | 247 | −25.059 | −27.489 | 33.461 | 1.00 | 41.29 | A |
| ATOM | 1982 | CB | THR | A | 247 | −24.660 | −27.064 | 34.890 | 1.00 | 43.90 | A |
| ATOM | 1983 | OG1 | THR | A | 247 | −24.136 | −25.733 | 34.868 | 1.00 | 41.14 | A |
| ATOM | 1984 | CG2 | THR | A | 247 | −23.623 | −28.013 | 35.464 | 1.00 | 41.14 | A |
| ATOM | 1985 | C | THR | A | 247 | −23.820 | −27.715 | 32.608 | 1.00 | 41.29 | A |
| ATOM | 1986 | O | THR | A | 247 | −23.321 | −28.834 | 32.535 | 1.00 | 41.29 | A |
| ATOM | 1987 | N | GLY | A | 248 | −23.340 | −26.666 | 31.953 | 1.00 | 29.23 | A |
| ATOM | 1988 | CA | GLY | A | 248 | −22.181 | −26.796 | 31.088 | 1.00 | 29.23 | A |
| ATOM | 1989 | C | GLY | A | 248 | −22.021 | −25.594 | 30.173 | 1.00 | 29.23 | A |
| ATOM | 1990 | O | GLY | A | 248 | −22.716 | −24.585 | 30.327 | 1.00 | 29.23 | A |
| ATOM | 1991 | N | CYS | A | 249 | −21.105 | −25.678 | 29.222 | 1.00 | 40.32 | A |
| ATOM | 1992 | CA | CYS | A | 249 | −20.886 | −24.560 | 28.312 | 1.00 | 40.32 | A |
| ATOM | 1993 | CB | CYS | A | 249 | −19.808 | −23.618 | 28.852 | 1.00 | 28.15 | A |
| ATOM | 1994 | SG | CYS | A | 249 | −18.108 | −24.253 | 28.735 | 1.00 | 46.92 | A |
| ATOM | 1995 | C | CYS | A | 249 | −20.485 | −25.038 | 26.923 | 1.00 | 40.32 | A |
| ATOM | 1996 | O | CYS | A | 249 | −20.347 | −26.242 | 26.684 | 1.00 | 40.32 | A |
| ATOM | 1997 | N | TYR | A | 250 | −20.290 | −24.093 | 26.008 | 1.00 | 28.82 | A |
| ATOM | 1998 | CA | TYR | A | 250 | −19.943 | −24.435 | 24.640 | 1.00 | 28.82 | A |
| ATOM | 1999 | CB | TYR | A | 250 | −21.208 | −24.804 | 23.874 | 1.00 | 40.60 | A |
| ATOM | 2000 | CG | TYR | A | 250 | −22.175 | −23.653 | 23.765 | 1.00 | 30.95 | A |
| ATOM | 2001 | CD1 | TYR | A | 250 | −22.109 | −22.756 | 22.697 | 1.00 | 30.95 | A |
| ATOM | 2002 | CE1 | TYR | A | 250 | −22.980 | −21.660 | 22.620 | 1.00 | 30.95 | A |
| ATOM | 2003 | CD2 | TYR | A | 250 | −23.133 | −23.431 | 24.755 | 1.00 | 30.95 | A |
| ATOM | 2004 | CE2 | TYR | A | 250 | −24.002 | −22.348 | 24.692 | 1.00 | 30.95 | A |
| ATOM | 2005 | CZ | TYR | A | 250 | −23.923 | −21.464 | 23.627 | 1.00 | 30.95 | A |
| ATOM | 2006 | OH | TYR | A | 250 | −24.764 | −20.374 | 23.596 | 1.00 | 30.95 | A |
| ATOM | 2007 | C | TYR | A | 250 | −19.254 | −23.305 | 23.899 | 1.00 | 28.82 | A |
| ATOM | 2008 | O | TYR | A | 250 | −19.429 | −22.132 | 24.218 | 1.00 | 28.82 | A |
| ATOM | 2009 | N | GLU | A | 251 | −18.472 | −23.702 | 22.901 | 1.00 | 55.77 | A |
| ATOM | 2010 | CA | GLU | A | 251 | −17.759 | −22.806 | 22.016 | 1.00 | 55.77 | A |
| ATOM | 2011 | CB | GLU | A | 251 | −16.274 | −22.779 | 22.347 | 1.00 | 122.74 | A |
| ATOM | 2012 | CG | GLU | A | 251 | −15.828 | −21.442 | 22.885 | 1.00 | 76.36 | A |
| ATOM | 2013 | CD | GLU | A | 251 | −14.319 | −21.340 | 23.060 | 1.00 | 76.36 | A |
| ATOM | 2014 | OE1 | GLU | A | 251 | −13.580 | −21.568 | 22.074 | 1.00 | 76.36 | A |
| ATOM | 2015 | OE2 | GLU | A | 251 | −13.869 | −21.021 | 24.185 | 1.00 | 76.36 | A |
| ATOM | 2016 | C | GLU | A | 251 | −17.992 | −23.443 | 20.652 | 1.00 | 55.77 | A |
| ATOM | 2017 | O | GLU | A | 251 | −17.161 | −24.191 | 20.154 | 1.00 | 55.77 | A |
| ATOM | 2018 | N | THR | A | 252 | −19.162 | −23.174 | 20.081 | 1.00 | 42.72 | A |
| ATOM | 2019 | CA | THR | A | 252 | −19.570 | −23.692 | 18.776 | 1.00 | 42.72 | A |
| ATOM | 2020 | CB | THR | A | 252 | −21.080 | −23.968 | 18.766 | 1.00 | 48.59 | A |
| ATOM | 2021 | OG1 | THR | A | 252 | −21.408 | −24.809 | 19.878 | 1.00 | 48.59 | A |
| ATOM | 2022 | CG2 | THR | A | 252 | −21.500 | −24.655 | 17.479 | 1.00 | 48.59 | A |
| ATOM | 2023 | C | THR | A | 252 | −19.260 | −22.632 | 17.723 | 1.00 | 42.72 | A |
| ATOM | 2024 | O | THR | A | 252 | −20.060 | −21.725 | 17.503 | 1.00 | 42.72 | A |
| ATOM | 2025 | N | GLY | A | 253 | −18.107 | −22.740 | 17.071 | 1.00 | 46.62 | A |
| ATOM | 2026 | CA | GLY | A | 253 | −17.746 | −21.734 | 16.091 | 1.00 | 46.62 | A |
| ATOM | 2027 | C | GLY | A | 253 | −17.601 | −20.395 | 16.797 | 1.00 | 46.62 | A |
| ATOM | 2028 | O | GLY | A | 253 | −16.918 | −20.298 | 17.821 | 1.00 | 46.62 | A |
| ATOM | 2029 | N | LEU | A | 254 | −18.251 | −19.367 | 16.265 | 1.00 | 41.88 | A |
| ATOM | 2030 | CA | LEU | A | 254 | −18.193 | −18.045 | 16.866 | 1.00 | 41.88 | A |
| ATOM | 2031 | CB | LEU | A | 254 | −18.667 | −16.978 | 15.880 | 1.00 | 41.55 | A |
| ATOM | 2032 | CG | LEU | A | 254 | −17.799 | −16.639 | 14.678 | 1.00 | 41.55 | A |
| ATOM | 2033 | CD1 | LEU | A | 254 | −18.413 | −15.434 | 13.983 | 1.00 | 41.55 | A |
| ATOM | 2034 | CD2 | LEU | A | 254 | −16.368 | −16.346 | 15.120 | 1.00 | 41.55 | A |
| ATOM | 2035 | C | LEU | A | 254 | −19.062 | −17.954 | 18.110 | 1.00 | 41.88 | A |
| ATOM | 2036 | O | LEU | A | 254 | −18.769 | −17.171 | 19.013 | 1.00 | 41.88 | A |
| ATOM | 2037 | N | LEU | A | 255 | −20.136 | −18.739 | 18.148 | 1.00 | 39.50 | A |
| ATOM | 2038 | CA | LEU | A | 255 | −21.045 | −18.718 | 19.284 | 1.00 | 39.50 | A |
| ATOM | 2039 | CB | LEU | A | 255 | −22.335 | −19.458 | 18.945 | 1.00 | 34.93 | A |
| ATOM | 2040 | CG | LEU | A | 255 | −23.024 | −19.026 | 17.654 | 1.00 | 34.93 | A |
| ATOM | 2041 | CD1 | LEU | A | 255 | −24.331 | −19.802 | 17.505 | 1.00 | 34.93 | A |
| ATOM | 2042 | CD2 | LEU | A | 255 | −23.290 | −17.535 | 17.671 | 1.00 | 34.93 | A |
| ATOM | 2043 | C | LEU | A | 255 | −20.429 | −19.330 | 20.538 | 1.00 | 39.50 | A |
| ATOM | 2044 | O | LEU | A | 255 | −19.582 | −20.224 | 20.459 | 1.00 | 39.50 | A |
| ATOM | 2045 | N | PHE | A | 256 | −20.851 | −18.832 | 21.698 | 1.00 | 36.06 | A |
| ATOM | 2046 | CA | PHE | A | 256 | −20.362 | −19.353 | 22.962 | 1.00 | 36.06 | A |
| ATOM | 2047 | CB | PHE | A | 256 | −18.945 | −18.836 | 23.228 | 1.00 | 41.07 | A |
| ATOM | 2048 | CG | PHE | A | 256 | −18.862 | −17.350 | 23.439 | 1.00 | 38.08 | A |
| ATOM | 2049 | CD1 | PHE | A | 256 | −18.856 | −16.817 | 24.716 | 1.00 | 38.08 | A |
| ATOM | 2050 | CD2 | PHE | A | 256 | −18.779 | −16.475 | 22.356 | 1.00 | 38.08 | A |
| ATOM | 2051 | CE1 | PHE | A | 256 | −18.766 | −15.442 | 24.913 | 1.00 | 38.08 | A |
| ATOM | 2052 | CE2 | PHE | A | 256 | −18.689 | −15.091 | 22.554 | 1.00 | 38.08 | A |
| ATOM | 2053 | CZ | PHE | A | 256 | −18.682 | −14.584 | 23.830 | 1.00 | 38.08 | A |
| ATOM | 2054 | C | PHE | A | 256 | −21.305 | −18.961 | 24.087 | 1.00 | 36.06 | A |
| ATOM | 2055 | O | PHE | A | 256 | −21.889 | −17.883 | 24.059 | 1.00 | 36.06 | A |
| ATOM | 2056 | N | GLY | A | 257 | −21.467 | −19.848 | 25.064 | 1.00 | 49.71 | A |
| ATOM | 2057 | CA | GLY | A | 257 | −22.342 | −19.557 | 26.184 | 1.00 | 49.71 | A |
| ATOM | 2058 | C | GLY | A | 257 | −22.303 | −20.650 | 27.234 | 1.00 | 49.71 | A |
| ATOM | 2059 | O | GLY | A | 257 | −21.567 | −21.629 | 27.087 | 1.00 | 49.71 | A |
| ATOM | 2060 | N | SER | A | 258 | −23.086 | −20.490 | 28.298 | 1.00 | 35.40 | A |
| ATOM | 2061 | CA | SER | A | 258 | −23.130 | −21.488 | 29.360 | 1.00 | 35.40 | A |
| ATOM | 2062 | CB | SER | A | 258 | −22.272 | −21.050 | 30.542 | 1.00 | 41.52 | A |
| ATOM | 2063 | OG | SER | A | 258 | −22.838 | −19.925 | 31.180 | 1.00 | 51.64 | A |
| ATOM | 2064 | C | SER | A | 258 | −24.571 | −21.670 | 29.811 | 1.00 | 35.40 | A |
| ATOM | 2065 | O | SER | A | 258 | −25.416 | −20.818 | 29.545 | 1.00 | 35.40 | A |
| ATOM | 2066 | N | TYR | A | 259 | −24.856 | −22.762 | 30.504 | 1.00 | 41.55 | A |
| ATOM | 2067 | CA | TYR | A | 259 | −26.217 | −23.002 | 30.944 | 1.00 | 41.55 | A |
| ATOM | 2068 | CB | TYR | A | 259 | −26.975 | −23.785 | 29.864 | 1.00 | 46.65 | A |
| ATOM | 2069 | CG | TYR | A | 259 | −26.239 | −25.033 | 29.431 | 1.00 | 42.40 | A |
| ATOM | 2070 | CD1 | TYR | A | 259 | −26.313 | −26.211 | 30.177 | 1.00 | 42.40 | A |
| ATOM | 2071 | CE1 | TYR | A | 259 | −25.560 | −27.314 | 29.839 | 1.00 | 42.40 | A |
| ATOM | 2072 | CD2 | TYR | A | 259 | −25.388 | −25.008 | 28.329 | 1.00 | 42.40 | A |
| ATOM | 2073 | CE2 | TYR | A | 259 | −24.628 | −26.118 | 27.990 | 1.00 | 42.40 | A |
| ATOM | 2074 | CZ | TYR | A | 259 | −24.722 | −27.258 | 28.751 | 1.00 | 42.40 | A |
| ATOM | 2075 | OH | TYR | A | 259 | −23.952 | −28.336 | 28.421 | 1.00 | 42.40 | A |
| ATOM | 2076 | C | TYR | A | 259 | −26.235 | −23.775 | 32.241 | 1.00 | 41.55 | A |
| ATOM | 2077 | O | TYR | A | 259 | −25.236 | −24.371 | 32.637 | 1.00 | 41.55 | A |
| ATOM | 2078 | N | GLY | A | 260 | −27.389 | −23.765 | 32.895 | 1.00 | 43.69 | A |
| ATOM | 2079 | CA | GLY | A | 260 | −27.564 | −24.479 | 34.139 | 1.00 | 43.69 | A |
| ATOM | 2080 | C | GLY | A | 260 | −29.047 | −24.510 | 34.389 | 1.00 | 43.69 | A |
| ATOM | 2081 | O | GLY | A | 260 | −29.634 | −23.488 | 34.736 | 1.00 | 43.69 | A |
| ATOM | 2082 | N | TYR | A | 261 | −29.666 | −25.662 | 34.187 | 1.00 | 38.05 | A |
| ATOM | 2083 | CA | TYR | A | 261 | −31.091 | −25.787 | 34.413 | 1.00 | 38.05 | A |
| ATOM | 2084 | CB | TYR | A | 261 | −31.837 | −25.598 | 33.106 | 1.00 | 52.66 | A |
| ATOM | 2085 | CG | TYR | A | 261 | −31.492 | −26.613 | 32.044 | 1.00 | 36.45 | A |
| ATOM | 2086 | CD1 | TYR | A | 261 | −32.166 | −27.838 | 31.966 | 1.00 | 36.45 | A |
| ATOM | 2087 | CE1 | TYR | A | 261 | −31.911 | −28.732 | 30.932 | 1.00 | 36.45 | A |
| ATOM | 2088 | CD2 | TYR | A | 261 | −30.540 | −26.320 | 31.064 | 1.00 | 36.45 | A |
| ATOM | 2089 | CE2 | TYR | A | 261 | −30.272 | −27.212 | 30.024 | 1.00 | 36.45 | A |
| ATOM | 2090 | CZ | TYR | A | 261 | −30.962 | −28.409 | 29.962 | 1.00 | 36.45 | A |
| ATOM | 2091 | OH | TYR | A | 261 | −30.718 | −29.267 | 28.917 | 1.00 | 36.45 | A |
| ATOM | 2092 | C | TYR | A | 261 | −31.444 | −27.125 | 35.038 | 1.00 | 38.05 | A |
| ATOM | 2093 | O | TYR | A | 261 | −30.628 | −28.050 | 35.065 | 1.00 | 38.05 | A |
| ATOM | 2094 | N | VAL | A | 262 | −32.665 | −27.213 | 35.551 | 1.00 | 46.88 | A |
| ATOM | 2095 | CA | VAL | A | 262 | −33.144 | −28.416 | 36.196 | 1.00 | 46.88 | A |
| ATOM | 2096 | CB | VAL | A | 262 | −33.636 | −28.099 | 37.630 | 1.00 | 35.20 | A |
| ATOM | 2097 | CG1 | VAL | A | 262 | −34.379 | −29.295 | 38.220 | 1.00 | 26.69 | A |
| ATOM | 2098 | CG2 | VAL | A | 262 | −32.458 | −27.735 | 38.509 | 1.00 | 26.69 | A |
| ATOM | 2099 | C | VAL | A | 262 | −34.297 | −28.958 | 35.388 | 1.00 | 46.88 | A |
| ATOM | 2100 | O | VAL | A | 262 | −35.240 | −28.230 | 35.119 | 1.00 | 46.88 | A |
| ATOM | 2101 | N | GLU | A | 263 | −34.213 | −30.219 | 34.974 | 1.00 | 39.93 | A |
| ATOM | 2102 | CA | GLU | A | 263 | −35.314 | −30.834 | 34.247 | 1.00 | 39.93 | A |
| ATOM | 2103 | CB | GLU | A | 263 | −34.921 | −31.215 | 32.823 | 1.00 | 80.13 | A |
| ATOM | 2104 | CG | GLU | A | 263 | −33.723 | −32.091 | 32.721 | 1.00 | 66.73 | A |
| ATOM | 2105 | CD | GLU | A | 263 | −33.407 | −32.447 | 31.285 | 1.00 | 66.73 | A |
| ATOM | 2106 | OE1 | GLU | A | 263 | −33.528 | −31.552 | 30.409 | 1.00 | 66.73 | A |
| ATOM | 2107 | OE2 | GLU | A | 263 | −33.024 | −33.617 | 31.034 | 1.00 | 66.73 | A |
| ATOM | 2108 | C | GLU | A | 263 | −35.830 | −32.055 | 35.007 | 1.00 | 39.93 | A |
| ATOM | 2109 | O | GLU | A | 263 | −35.146 | −32.621 | 35.859 | 1.00 | 39.93 | A |
| ATOM | 2110 | N | THR | A | 264 | −37.058 | −32.435 | 34.683 | 1.00 | 61.03 | A |
| ATOM | 2111 | CA | THR | A | 264 | −37.753 | −33.543 | 35.311 | 1.00 | 61.03 | A |
| ATOM | 2112 | CB | THR | A | 264 | −39.066 | −33.008 | 35.928 | 1.00 | 85.07 | A |
| ATOM | 2113 | OG1 | THR | A | 264 | −38.876 | −32.797 | 37.329 | 1.00 | 64.00 | A |
| ATOM | 2114 | CG2 | THR | A | 264 | −40.222 | −33.933 | 35.691 | 1.00 | 64.00 | A |
| ATOM | 2115 | C | THR | A | 264 | −38.031 | −34.598 | 34.260 | 1.00 | 61.03 | A |
| ATOM | 2116 | O | THR | A | 264 | −37.873 | −34.340 | 33.075 | 1.00 | 61.03 | A |
| ATOM | 2117 | N | ALA | A | 265 | −38.418 | −35.794 | 34.680 | 1.00 | 60.02 | A |
| ATOM | 2118 | CA | ALA | A | 265 | −38.725 | −36.842 | 33.711 | 1.00 | 60.02 | A |
| ATOM | 2119 | CB | ALA | A | 265 | −39.104 | −38.132 | 34.435 | 1.00 | 89.86 | A |
| ATOM | 2120 | C | ALA | A | 265 | −39.891 | −36.353 | 32.832 | 1.00 | 60.02 | A |
| ATOM | 2121 | O | ALA | A | 265 | −40.075 | −36.822 | 31.708 | 1.00 | 60.02 | A |
| ATOM | 2122 | N | ASN | A | 266 | −40.660 | −35.395 | 33.355 | 1.00 | 69.57 | A |
| ATOM | 2123 | CA | ASN | A | 266 | −41.807 | −34.825 | 32.655 | 1.00 | 69.57 | A |
| ATOM | 2124 | CB | ASN | A | 266 | −42.836 | −34.298 | 33.652 | 1.00 | 138.29 | A |
| ATOM | 2125 | CG | ASN | A | 266 | −43.261 | −35.344 | 34.646 | 1.00 | 80.14 | A |
| ATOM | 2126 | OD1 | ASN | A | 266 | −43.640 | −36.459 | 34.272 | 1.00 | 80.14 | A |
| ATOM | 2127 | ND2 | ASN | A | 266 | −43.202 | −34.997 | 35.929 | 1.00 | 80.14 | A |
| ATOM | 2128 | C | ASN | A | 266 | −41.410 | −33.696 | 31.714 | 1.00 | 69.57 | A |
| ATOM | 2129 | O | ASN | A | 266 | −42.206 | −33.279 | 30.871 | 1.00 | 69.57 | A |
| ATOM | 2130 | N | GLY | A | 267 | −40.185 | −33.194 | 31.860 | 1.00 | 67.66 | A |
| ATOM | 2131 | CA | GLY | A | 267 | −39.719 | −32.117 | 30.999 | 1.00 | 67.66 | A |
| ATOM | 2132 | C | GLY | A | 267 | −39.017 | −30.991 | 31.732 | 1.00 | 67.66 | A |
| ATOM | 2133 | O | GLY | A | 267 | −38.987 | −30.981 | 32.957 | 1.00 | 67.66 | A |
| ATOM | 2134 | N | PRO | A | 268 | −38.451 | −30.014 | 31.006 | 1.00 | 56.39 | A |
| ATOM | 2135 | CD | PRO | A | 268 | −38.666 | −29.792 | 29.570 | 1.00 | 45.81 | A |
| ATOM | 2136 | CA | PRO | A | 268 | −37.741 | −28.873 | 31.595 | 1.00 | 56.39 | A |
| ATOM | 2137 | CB | PRO | A | 268 | −37.524 | −27.945 | 30.407 | 1.00 | 61.70 | A |
| ATOM | 2138 | CG | PRO | A | 268 | −38.653 | −28.292 | 29.499 | 1.00 | 45.81 | A |
| ATOM | 2139 | C | PRO | A | 268 | −38.533 | −28.215 | 32.701 | 1.00 | 56.39 | A |
| ATOM | 2140 | O | PRO | A | 268 | −39.732 | −27.993 | 32.566 | 1.00 | 56.39 | A |
| ATOM | 2141 | N | LEU | A | 269 | −37.848 | −27.896 | 33.792 | 1.00 | 58.92 | A |
| ATOM | 2142 | CA | LEU | A | 269 | −38.481 | −27.293 | 34.955 | 1.00 | 58.92 | A |
| ATOM | 2143 | CB | LEU | A | 269 | −38.136 | −28.130 | 36.186 | 1.00 | 88.77 | A |
| ATOM | 2144 | CG | LEU | A | 269 | −38.972 | −27.909 | 37.429 | 1.00 | 54.11 | A |
| ATOM | 2145 | CD1 | LEU | A | 269 | −40.430 | −28.090 | 37.074 | 1.00 | 54.11 | A |
| ATOM | 2146 | CD2 | LEU | A | 269 | −38.554 | −28.884 | 38.502 | 1.00 | 54.11 | A |
| ATOM | 2147 | C | LEU | A | 269 | −38.068 | −25.829 | 35.160 | 1.00 | 58.92 | A |
| ATOM | 2148 | O | LEU | A | 269 | −38.860 | −24.917 | 34.916 | 1.00 | 58.92 | A |
| ATOM | 2149 | N | ALA | A | 270 | −36.834 | −25.601 | 35.601 | 1.00 | 57.43 | A |
| ATOM | 2150 | CA | ALA | A | 270 | −36.346 | −24.243 | 35.841 | 1.00 | 57.43 | A |
| ATOM | 2151 | CB | ALA | A | 270 | −36.438 | −23.898 | 37.318 | 1.00 | 36.48 | A |
| ATOM | 2152 | C | ALA | A | 270 | −34.924 | −24.020 | 35.371 | 1.00 | 57.43 | A |
| ATOM | 2153 | O | ALA | A | 270 | −34.073 | −24.901 | 35.457 | 1.00 | 57.43 | A |
| ATOM | 2154 | N | ARG | A | 271 | −34.672 | −22.803 | 34.913 | 1.00 | 48.73 | A |
| ATOM | 2155 | CA | ARG | A | 271 | −33.372 | −22.380 | 34.388 | 1.00 | 48.73 | A |
| ATOM | 2156 | CB | ARG | A | 271 | −33.604 | −21.774 | 33.003 | 1.00 | 55.45 | A |
| ATOM | 2157 | CG | ARG | A | 271 | −32.454 | −21.024 | 32.402 | 1.00 | 48.57 | A |
| ATOM | 2158 | CD | ARG | A | 271 | −32.868 | −20.390 | 31.074 | 1.00 | 48.57 | A |
| ATOM | 2159 | NE | ARG | A | 271 | −33.093 | −21.373 | 30.022 | 1.00 | 48.57 | A |
| ATOM | 2160 | CZ | ARG | A | 271 | −34.287 | −21.716 | 29.554 | 1.00 | 48.57 | A |
| ATOM | 2161 | NH1 | ARG | A | 271 | −35.378 | −21.149 | 30.046 | 1.00 | 48.57 | A |
| ATOM | 2162 | NH2 | ARG | A | 271 | −34.384 | −22.628 | 28.593 | 1.00 | 48.57 | A |
| ATOM | 2163 | C | ARG | A | 271 | −32.740 | −21.361 | 35.335 | 1.00 | 48.73 | A |
| ATOM | 2164 | O | ARG | A | 271 | −33.411 | −20.456 | 35.817 | 1.00 | 48.73 | A |
| ATOM | 2165 | N | TYR | A | 272 | −31.452 | −21.508 | 35.608 | 1.00 | 49.18 | A |
| ATOM | 2166 | CA | TYR | A | 272 | −30.773 | −20.602 | 36.528 | 1.00 | 49.18 | A |
| ATOM | 2167 | CB | TYR | A | 272 | −29.679 | −21.338 | 37.291 | 1.00 | 40.45 | A |
| ATOM | 2168 | CG | TYR | A | 272 | −28.959 | −20.476 | 38.301 | 1.00 | 37.92 | A |
| ATOM | 2169 | CD1 | TYR | A | 272 | −29.511 | −20.231 | 39.561 | 1.00 | 37.92 | A |
| ATOM | 2170 | CE1 | TYR | A | 272 | −28.876 | −19.374 | 40.476 | 1.00 | 37.92 | A |
| ATOM | 2171 | CD2 | TYR | A | 272 | −27.748 | −19.850 | 37.983 | 1.00 | 37.92 | A |
| ATOM | 2172 | CE2 | TYR | A | 272 | −27.113 | −18.998 | 38.893 | 1.00 | 37.92 | A |
| ATOM | 2173 | CZ | TYR | A | 272 | −27.687 | −18.769 | 40.129 | 1.00 | 37.92 | A |
| ATOM | 2174 | OH | TYR | A | 272 | −27.082 | −17.929 | 41.022 | 1.00 | 37.92 | A |
| ATOM | 2175 | C | TYR | A | 272 | −30.148 | −19.433 | 35.800 | 1.00 | 49.18 | A |
| ATOM | 2176 | O | TYR | A | 272 | −29.317 | −19.626 | 34.918 | 1.00 | 49.18 | A |
| ATOM | 2177 | N | HIS | A | 273 | −30.536 | −18.217 | 36.174 | 1.00 | 70.65 | A |
| ATOM | 2178 | CA | HIS | A | 273 | −29.992 | −17.023 | 35.533 | 1.00 | 70.65 | A |
| ATOM | 2179 | CB | HIS | A | 273 | −31.104 | −16.018 | 35.222 | 1.00 | 133.16 | A |
| ATOM | 2180 | CG | HIS | A | 273 | −31.958 | −16.419 | 34.060 | 1.00 | 113.89 | A |
| ATOM | 2181 | CD2 | HIS | A | 273 | −33.284 | −16.678 | 33.972 | 1.00 | 113.89 | A |
| ATOM | 2182 | ND1 | HIS | A | 273 | −31.442 | −16.630 | 32.800 | 1.00 | 113.89 | A |
| ATOM | 2183 | CE1 | HIS | A | 273 | −32.413 | −17.002 | 31.985 | 1.00 | 113.89 | A |
| ATOM | 2184 | NE2 | HIS | A | 273 | −33.541 | −17.040 | 32.672 | 1.00 | 113.89 | A |
| ATOM | 2185 | C | HIS | A | 273 | −28.901 | −16.361 | 36.347 | 1.00 | 70.65 | A |
| ATOM | 2186 | O | HIS | A | 273 | −29.101 | −15.945 | 37.486 | 1.00 | 70.65 | A |
| ATOM | 2187 | N | THR | A | 274 | −27.730 | −16.289 | 35.733 | 1.00 | 59.27 | A |
| ATOM | 2188 | CA | THR | A | 274 | −26.551 | −15.691 | 36.333 | 1.00 | 59.27 | A |
| ATOM | 2189 | CB | THR | A | 274 | −25.387 | −15.752 | 35.334 | 1.00 | 62.69 | A |
| ATOM | 2190 | OG1 | THR | A | 274 | −24.586 | −16.900 | 35.626 | 1.00 | 55.22 | A |
| ATOM | 2191 | CG2 | THR | A | 274 | −24.550 | −14.493 | 35.384 | 1.00 | 55.22 | A |
| ATOM | 2192 | C | THR | A | 274 | −26.789 | −14.251 | 36.763 | 1.00 | 59.27 | A |
| ATOM | 2193 | O | THR | A | 274 | −26.265 | −13.804 | 37.782 | 1.00 | 59.27 | A |
| ATOM | 2194 | N | GLY | A | 275 | −27.577 | −13.523 | 35.980 | 1.00 | 54.93 | A |
| ATOM | 2195 | CA | GLY | A | 275 | −27.864 | −12.139 | 36.305 | 1.00 | 54.93 | A |
| ATOM | 2196 | C | GLY | A | 275 | −28.712 | −11.969 | 37.553 | 1.00 | 54.93 | A |
| ATOM | 2197 | O | GLY | A | 275 | −28.263 | −11.400 | 38.548 | 1.00 | 54.93 | A |
| ATOM | 2198 | N | ASP | A | 276 | −29.942 | −12.469 | 37.502 | 1.00 | 58.31 | A |
| ATOM | 2199 | CA | ASP | A | 276 | −30.869 | −12.374 | 38.626 | 1.00 | 58.31 | A |
| ATOM | 2200 | CB | ASP | A | 276 | −32.256 | −12.819 | 38.170 | 1.00 | 146.35 | A |
| ATOM | 2201 | CG | ASP | A | 276 | −32.677 | −12.155 | 36.886 | 1.00 | 125.35 | A |
| ATOM | 2202 | OD1 | ASP | A | 276 | −32.914 | −10.929 | 36.905 | 1.00 | 125.35 | A |
| ATOM | 2203 | OD2 | ASP | A | 276 | −32.759 | −12.858 | 35.854 | 1.00 | 125.35 | A |
| ATOM | 2204 | C | ASP | A | 276 | −30.458 | −13.205 | 39.845 | 1.00 | 58.31 | A |
| ATOM | 2205 | O | ASP | A | 276 | −30.951 | −12.966 | 40.948 | 1.00 | 58.31 | A |
| ATOM | 2206 | N | LYS | A | 277 | −29.571 | −14.181 | 39.649 | 1.00 | 54.13 | A |
| ATOM | 2207 | CA | LYS | A | 277 | −29.129 | −15.052 | 40.738 | 1.00 | 54.13 | A |
| ATOM | 2208 | CB | LYS | A | 277 | −28.400 | −14.235 | 41.807 | 1.00 | 90.93 | A |
| ATOM | 2209 | CG | LYS | A | 277 | −27.083 | −13.659 | 41.335 | 1.00 | 76.14 | A |
| ATOM | 2210 | CD | LYS | A | 277 | −26.584 | −12.592 | 42.288 | 1.00 | 76.14 | A |
| ATOM | 2211 | CE | LYS | A | 277 | −25.220 | −12.043 | 41.871 | 1.00 | 76.14 | A |
| ATOM | 2212 | NZ | LYS | A | 277 | −24.085 | −12.982 | 42.165 | 1.00 | 76.14 | A |
| ATOM | 2213 | C | LYS | A | 277 | −30.323 | −15.799 | 41.352 | 1.00 | 54.13 | A |
| ATOM | 2214 | O | LYS | A | 277 | −30.514 | −15.818 | 42.564 | 1.00 | 54.13 | A |
| ATOM | 2215 | N | ARG | A | 278 | −31.122 | −16.415 | 40.492 | 1.00 | 57.61 | A |
| ATOM | 2216 | CA | ARG | A | 278 | −32.291 | −17.169 | 40.920 | 1.00 | 57.61 | A |
| ATOM | 2217 | CB | ARG | A | 278 | −33.402 | −16.210 | 41.327 | 1.00 | 127.22 | A |
| ATOM | 2218 | CG | ARG | A | 278 | −33.984 | −15.471 | 40.149 | 1.00 | 111.85 | A |
| ATOM | 2219 | CD | ARG | A | 278 | −34.884 | −14.342 | 40.581 | 1.00 | 111.85 | A |
| ATOM | 2220 | NE | ARG | A | 278 | −35.537 | −13.710 | 39.436 | 1.00 | 111.85 | A |
| ATOM | 2221 | CZ | ARG | A | 278 | −36.152 | −12.532 | 39.481 | 1.00 | 111.85 | A |
| ATOM | 2222 | NH1 | ARG | A | 278 | −36.197 | −11.850 | 40.619 | 1.00 | 111.85 | A |
| ATOM | 2223 | NH2 | ARG | A | 278 | −36.723 | −12.037 | 38.389 | 1.00 | 111.85 | A |
| ATOM | 2224 | C | ARG | A | 278 | −32.766 | −18.032 | 39.745 | 1.00 | 57.61 | A |
| ATOM | 2225 | O | ARG | A | 278 | −32.301 | −17.861 | 38.617 | 1.00 | 57.61 | A |
| ATOM | 2226 | N | PHE | A | 279 | −33.679 | −18.963 | 40.006 | 1.00 | 52.63 | A |
| ATOM | 2227 | CA | PHE | A | 279 | −34.200 | −19.808 | 38.943 | 1.00 | 52.63 | A |
| ATOM | 2228 | CB | PHE | A | 279 | −34.638 | −21.170 | 39.486 | 1.00 | 61.73 | A |
| ATOM | 2229 | CG | PHE | A | 279 | −33.513 | −22.135 | 39.695 | 1.00 | 45.50 | A |
| ATOM | 2230 | CD1 | PHE | A | 279 | −32.863 | −22.211 | 40.918 | 1.00 | 45.50 | A |
| ATOM | 2231 | CD2 | PHE | A | 279 | −33.091 | −22.960 | 38.661 | 1.00 | 45.50 | A |
| ATOM | 2232 | CE1 | PHE | A | 279 | −31.802 | −23.099 | 41.109 | 1.00 | 45.50 | A |
| ATOM | 2233 | CE2 | PHE | A | 279 | −32.036 | −23.845 | 38.844 | 1.00 | 45.50 | A |
| ATOM | 2234 | CZ | PHE | A | 279 | −31.389 | −23.915 | 40.070 | 1.00 | 45.50 | A |
| ATOM | 2235 | C | PHE | A | 279 | −35.398 | −19.142 | 38.277 | 1.00 | 52.63 | A |
| ATOM | 2236 | O | PHE | A | 279 | −36.089 | −18.331 | 38.895 | 1.00 | 52.63 | A |
| ATOM | 2237 | N | THR | A | 280 | −35.639 | −19.488 | 37.015 | 1.00 | 48.01 | A |
| ATOM | 2238 | CA | THR | A | 280 | −36.770 | −18.950 | 36.278 | 1.00 | 48.01 | A |
| ATOM | 2239 | CB | THR | A | 280 | −36.334 | −17.912 | 35.265 | 1.00 | 92.88 | A |
| ATOM | 2240 | OG1 | THR | A | 280 | −35.499 | −16.950 | 35.910 | 1.00 | 73.40 | A |
| ATOM | 2241 | CG2 | THR | A | 280 | −37.549 | −17.213 | 34.677 | 1.00 | 73.40 | A |
| ATOM | 2242 | C | THR | A | 280 | −37.464 | −20.067 | 35.521 | 1.00 | 48.01 | A |
| ATOM | 2243 | O | THR | A | 280 | −36.804 | −20.926 | 34.942 | 1.00 | 48.01 | A |
| ATOM | 2244 | N | LYS | A | 281 | −38.794 | −20.047 | 35.528 | 1.00 | 53.03 | A |
| ATOM | 2245 | CA | LYS | A | 281 | −39.594 | −21.050 | 34.833 | 1.00 | 53.03 | A |
| ATOM | 2246 | CB | LYS | A | 281 | −41.064 | −20.629 | 34.854 | 1.00 | 106.23 | A |
| ATOM | 2247 | CG | LYS | A | 281 | −41.252 | −19.130 | 34.716 | 1.00 | 82.17 | A |
| ATOM | 2248 | CD | LYS | A | 281 | −42.275 | −18.765 | 33.650 | 1.00 | 82.17 | A |
| ATOM | 2249 | CE | LYS | A | 281 | −43.689 | −19.201 | 34.023 | 1.00 | 82.17 | A |
| ATOM | 2250 | NZ | LYS | A | 281 | −44.700 | −18.779 | 32.993 | 1.00 | 82.17 | A |
| ATOM | 2251 | C | LYS | A | 281 | −39.132 | −21.245 | 33.388 | 1.00 | 53.03 | A |
| ATOM | 2252 | O | LYS | A | 281 | −38.735 | −20.292 | 32.719 | 1.00 | 53.03 | A |
| ATOM | 2253 | N | MET | A | 282 | −39.162 | −22.488 | 32.920 | 1.00 | 46.21 | A |
| ATOM | 2254 | CA | MET | A | 282 | −38.775 | −22.794 | 31.546 | 1.00 | 46.21 | A |
| ATOM | 2255 | CB | MET | A | 282 | −37.836 | −24.000 | 31.483 | 1.00 | 60.91 | A |
| ATOM | 2256 | CG | MET | A | 282 | −36.443 | −23.744 | 32.022 | 1.00 | 60.91 | A |
| ATOM | 2257 | SD | MET | A | 282 | −35.284 | −25.020 | 31.505 | 1.00 | 60.91 | A |
| ATOM | 2258 | CE | MET | A | 282 | −35.491 | −26.194 | 32.787 | 1.00 | 60.91 | A |
| ATOM | 2259 | C | MET | A | 282 | −40.030 | −23.115 | 30.762 | 1.00 | 46.21 | A |
| ATOM | 2260 | O | MET | A | 282 | −39.991 | −23.233 | 29.542 | 1.00 | 46.21 | A |
| ATOM | 2261 | N | THR | A | 283 | −41.140 | −23.248 | 31.482 | 1.00 | 65.03 | A |
| ATOM | 2262 | CA | THR | A | 283 | −42.430 | −23.572 | 30.895 | 1.00 | 65.03 | A |
| ATOM | 2263 | CB | THR | A | 283 | −42.804 | −25.037 | 31.195 | 1.00 | 57.48 | A |
| ATOM | 2264 | OG1 | THR | A | 283 | −41.868 | −25.897 | 30.547 | 1.00 | 57.48 | A |
| ATOM | 2265 | CG2 | THR | A | 283 | −44.189 | −25.369 | 30.685 | 1.00 | 57.48 | A |
| ATOM | 2266 | C | THR | A | 283 | −43.480 | −22.670 | 31.511 | 1.00 | 65.03 | A |
| ATOM | 2267 | O | THR | A | 283 | −43.257 | −22.130 | 32.589 | 1.00 | 65.03 | A |
| ATOM | 2268 | N | HIS | A | 284 | −44.612 | −22.500 | 30.821 | 1.00 | 71.66 | A |
| ATOM | 2269 | CA | HIS | A | 284 | −45.726 | −21.688 | 31.317 | 1.00 | 71.66 | A |
| ATOM | 2270 | CB | HIS | A | 284 | −46.894 | −21.739 | 30.323 | 1.00 | 159.86 | A |
| ATOM | 2271 | CG | HIS | A | 284 | −47.372 | −23.129 | 30.020 | 1.00 | 159.86 | A |
| ATOM | 2272 | CD2 | HIS | A | 284 | −48.555 | −23.742 | 30.270 | 1.00 | 159.86 | A |
| ATOM | 2273 | ND1 | HIS | A | 284 | −46.582 | −24.071 | 29.394 | 1.00 | 159.86 | A |
| ATOM | 2274 | CE1 | HIS | A | 284 | −47.256 | −25.202 | 29.273 | 1.00 | 159.86 | A |
| ATOM | 2275 | NE2 | HIS | A | 284 | −48.456 | −25.029 | 29.797 | 1.00 | 159.86 | A |
| ATOM | 2276 | C | HIS | A | 284 | −46.180 | −22.260 | 32.671 | 1.00 | 71.66 | A |
| ATOM | 2277 | O | HIS | A | 284 | −46.545 | −21.518 | 33.591 | 1.00 | 71.66 | A |
| ATOM | 2278 | N | LYS | A | 285 | −46.125 | −23.589 | 32.769 | 1.00 | 92.53 | A |
| ATOM | 2279 | CA | LYS | A | 285 | −46.520 | −24.351 | 33.954 | 1.00 | 92.53 | A |
| ATOM | 2280 | CB | LYS | A | 285 | −46.138 | −25.826 | 33.765 | 1.00 | 141.03 | A |
| ATOM | 2281 | CG | LYS | A | 285 | −47.266 | −26.810 | 34.026 | 1.00 | 141.03 | A |
| ATOM | 2282 | CD | LYS | A | 285 | −48.396 | −26.643 | 33.013 | 1.00 | 141.03 | A |
| ATOM | 2283 | CE | LYS | A | 285 | −49.528 | −27.630 | 33.270 | 1.00 | 141.03 | A |
| ATOM | 2284 | NZ | LYS | A | 285 | −50.630 | −27.488 | 32.279 | 1.00 | 141.03 | A |
| ATOM | 2285 | C | LYS | A | 285 | −45.924 | −23.847 | 35.264 | 1.00 | 92.53 | A |
| ATOM | 2286 | O | LYS | A | 285 | −46.548 | −23.968 | 36.314 | 1.00 | 92.53 | A |
| ATOM | 2287 | N | GLY | A | 286 | −44.721 | −23.283 | 35.204 | 1.00 | 71.04 | A |
| ATOM | 2288 | CA | GLY | A | 286 | −44.067 | −22.799 | 36.410 | 1.00 | 71.04 | A |
| ATOM | 2289 | C | GLY | A | 286 | −43.319 | −23.937 | 37.086 | 1.00 | 71.04 | A |
| ATOM | 2290 | O | GLY | A | 286 | −43.283 | −25.056 | 36.568 | 1.00 | 71.04 | A |
| ATOM | 2291 | N | PHE | A | 287 | −42.711 | −23.668 | 38.235 | 1.00 | 69.53 | A |
| ATOM | 2292 | CA | PHE | A | 287 | −41.980 | −24.709 | 38.956 | 1.00 | 69.53 | A |
| ATOM | 2293 | CB | PHE | A | 287 | −40.492 | −24.678 | 38.590 | 1.00 | 59.33 | A |
| ATOM | 2294 | CG | PHE | A | 287 | −39.764 | −23.465 | 39.093 | 1.00 | 59.33 | A |
| ATOM | 2295 | CD1 | PHE | A | 287 | −39.244 | −23.434 | 40.383 | 1.00 | 59.33 | A |
| ATOM | 2296 | CD2 | PHE | A | 287 | −39.599 | −22.347 | 38.279 | 1.00 | 59.33 | A |
| ATOM | 2297 | CE1 | PHE | A | 287 | −38.566 | −22.302 | 40.856 | 1.00 | 59.33 | A |
| ATOM | 2298 | CE2 | PHE | A | 287 | −38.923 | −21.211 | 38.742 | 1.00 | 59.33 | A |
| ATOM | 2299 | CZ | PHE | A | 287 | −38.406 | −21.188 | 40.032 | 1.00 | 59.33 | A |
| ATOM | 2300 | C | PHE | A | 287 | −42.151 | −24.544 | 40.460 | 1.00 | 69.53 | A |
| ATOM | 2301 | O | PHE | A | 287 | −42.169 | −23.422 | 40.980 | 1.00 | 69.53 | A |
| ATOM | 2302 | N | PRO | A | 288 | −42.275 | −25.668 | 41.179 | 1.00 | 59.13 | A |
| ATOM | 2303 | CD | PRO | A | 288 | −42.166 | −27.044 | 40.666 | 1.00 | 42.83 | A |
| ATOM | 2304 | CA | PRO | A | 288 | −42.447 | −25.662 | 42.633 | 1.00 | 59.13 | A |
| ATOM | 2305 | CB | PRO | A | 288 | −42.690 | −27.132 | 42.953 | 1.00 | 42.83 | A |
| ATOM | 2306 | CG | PRO | A | 288 | −41.871 | −27.839 | 41.928 | 1.00 | 42.83 | A |
| ATOM | 2307 | C | PRO | A | 288 | −41.235 | −25.098 | 43.367 | 1.00 | 59.13 | A |
| ATOM | 2308 | O | PRO | A | 288 | −40.116 | −25.574 | 43.188 | 1.00 | 59.13 | A |
| ATOM | 2309 | N | LYS | A | 289 | −41.456 | −24.076 | 44.188 | 1.00 | 77.43 | A |
| ATOM | 2310 | CA | LYS | A | 289 | −40.363 | −23.473 | 44.934 | 1.00 | 77.43 | A |
| ATOM | 2311 | CB | LYS | A | 289 | −40.702 | −22.020 | 45.286 | 1.00 | 99.16 | A |
| ATOM | 2312 | CG | LYS | A | 289 | −40.539 | −21.074 | 44.094 | 1.00 | 99.16 | A |
| ATOM | 2313 | CD | LYS | A | 289 | −40.885 | −19.640 | 44.440 | 1.00 | 99.16 | A |
| ATOM | 2314 | CE | LYS | A | 289 | −42.382 | −19.477 | 44.659 | 1.00 | 99.16 | A |
| ATOM | 2315 | NZ | LYS | A | 289 | −42.762 | −18.071 | 44.974 | 1.00 | 99.16 | A |
| ATOM | 2316 | C | LYS | A | 289 | −40.033 | −24.290 | 46.180 | 1.00 | 77.43 | A |
| ATOM | 2317 | O | LYS | A | 289 | −40.406 | −23.941 | 47.299 | 1.00 | 77.43 | A |
| ATOM | 2318 | N | VAL | A | 290 | −39.312 | −25.385 | 45.955 | 1.00 | 66.58 | A |
| ATOM | 2319 | CA | VAL | A | 290 | −38.907 | −26.301 | 47.007 | 1.00 | 66.58 | A |
| ATOM | 2320 | CB | VAL | A | 290 | −39.789 | −27.562 | 47.005 | 1.00 | 55.78 | A |
| ATOM | 2321 | CG1 | VAL | A | 290 | −41.245 | −27.162 | 47.014 | 1.00 | 55.78 | A |
| ATOM | 2322 | CG2 | VAL | A | 290 | −39.483 | −28.410 | 45.782 | 1.00 | 55.78 | A |
| ATOM | 2323 | C | VAL | A | 290 | −37.466 | −26.734 | 46.782 | 1.00 | 66.58 | A |
| ATOM | 2324 | O | VAL | A | 290 | −36.896 | −26.485 | 45.723 | 1.00 | 66.58 | A |
| ATOM | 2325 | N | GLY | A | 291 | −36.892 | −27.395 | 47.781 | 1.00 | 51.99 | A |
| ATOM | 2326 | CA | GLY | A | 291 | −35.524 | −27.860 | 47.679 | 1.00 | 51.99 | A |
| ATOM | 2327 | C | GLY | A | 291 | −34.508 | −26.842 | 47.192 | 1.00 | 51.99 | A |
| ATOM | 2328 | O | GLY | A | 291 | −34.467 | −25.694 | 47.648 | 1.00 | 51.99 | A |
| ATOM | 2329 | N | MET | A | 292 | −33.682 | −27.280 | 46.249 | 1.00 | 59.90 | A |
| ATOM | 2330 | CA | MET | A | 292 | −32.636 | −26.450 | 45.672 | 1.00 | 59.90 | A |
| ATOM | 2331 | CB | MET | A | 292 | −31.713 | −27.307 | 44.820 | 1.00 | 60.79 | A |
| ATOM | 2332 | CG | MET | A | 292 | −32.410 | −27.800 | 43.569 | 1.00 | 60.79 | A |
| ATOM | 2333 | SD | MET | A | 292 | −31.290 | −28.366 | 42.287 | 1.00 | 60.79 | A |
| ATOM | 2334 | CE | MET | A | 292 | −31.702 | −30.095 | 42.244 | 1.00 | 60.79 | A |
| ATOM | 2335 | C | MET | A | 292 | −33.200 | −25.357 | 44.785 | 1.00 | 59.90 | A |
| ATOM | 2336 | O | MET | A | 292 | −32.446 | −24.576 | 44.212 | 1.00 | 59.90 | A |
| ATOM | 2337 | N | LEU | A | 293 | −34.519 | −25.300 | 44.664 | 1.00 | 73.36 | A |
| ATOM | 2338 | CA | LEU | A | 293 | −35.141 | −24.309 | 43.802 | 1.00 | 73.36 | A |
| ATOM | 2339 | CB | LEU | A | 293 | −36.302 | −24.956 | 43.052 | 1.00 | 46.30 | A |
| ATOM | 2340 | CG | LEU | A | 293 | −35.913 | −26.073 | 42.075 | 1.00 | 46.30 | A |
| ATOM | 2341 | CD1 | LEU | A | 293 | −37.165 | −26.708 | 41.492 | 1.00 | 46.30 | A |
| ATOM | 2342 | CD2 | LEU | A | 293 | −35.052 | −25.496 | 40.966 | 1.00 | 46.30 | A |
| ATOM | 2343 | C | LEU | A | 293 | −35.603 | −22.996 | 44.436 | 1.00 | 73.36 | A |
| ATOM | 2344 | O | LEU | A | 293 | −36.063 | −22.107 | 43.724 | 1.00 | 73.36 | A |
| ATOM | 2345 | N | THR | A | 294 | −35.488 | −22.854 | 45.754 | 1.00 | 71.51 | A |
| ATOM | 2346 | CA | THR | A | 294 | −35.903 | −21.603 | 46.390 | 1.00 | 71.51 | A |
| ATOM | 2347 | CB | THR | A | 294 | −37.088 | −21.780 | 47.325 | 1.00 | 88.17 | A |
| ATOM | 2348 | OG1 | THR | A | 294 | −38.015 | −22.698 | 46.747 | 1.00 | 88.17 | A |
| ATOM | 2349 | CG2 | THR | A | 294 | −37.782 | −20.436 | 47.539 | 1.00 | 88.17 | A |
| ATOM | 2350 | C | THR | A | 294 | −34.820 | −20.974 | 47.228 | 1.00 | 71.51 | A |
| ATOM | 2351 | O | THR | A | 294 | −33.978 | −21.663 | 47.793 | 1.00 | 71.51 | A |
| ATOM | 2352 | N | VAL | A | 295 | −34.858 | −19.652 | 47.322 | 1.00 | 72.74 | A |
| ATOM | 2353 | CA | VAL | A | 295 | −33.885 | −13.940 | 48.122 | 1.00 | 72.74 | A |
| ATOM | 2354 | CB | VAL | A | 295 | −34.015 | −17.432 | 47.907 | 1.00 | 90.46 | A |
| ATOM | 2355 | CG1 | VAL | A | 295 | −32.924 | −16.690 | 48.687 | 1.00 | 90.46 | A |
| ATOM | 2356 | CG2 | VAL | A | 295 | −33.930 | −17.126 | 46.421 | 1.00 | 90.46 | A |
| ATOM | 2357 | C | VAL | A | 295 | −34.156 | −19.284 | 49.587 | 1.00 | 72.74 | A |
| ATOM | 2358 | O | VAL | A | 295 | −33.281 | −19.140 | 50.448 | 1.00 | 72.74 | A |
| ATOM | 2359 | N | LYS | A | 296 | −35.373 | −19.756 | 49.851 | 1.00 | 63.22 | A |
| ATOM | 2360 | CA | LYS | A | 296 | −35.795 | −20.135 | 51.197 | 1.00 | 63.22 | A |
| ATOM | 2361 | CB | LYS | A | 296 | −37.241 | −20.642 | 51.170 | 1.00 | 108.60 | A |
| ATOM | 2362 | CG | LYS | A | 296 | −38.265 | −19.629 | 50.675 | 1.00 | 108.60 | A |
| ATOM | 2363 | CD | LYS | A | 296 | −38.532 | −18.531 | 51.702 | 1.00 | 108.60 | A |
| ATOM | 2364 | CE | LYS | A | 296 | −39.262 | −19.076 | 52.922 | 1.00 | 108.60 | A |
| ATOM | 2365 | NZ | LYS | A | 296 | −39.522 | −18.027 | 53.947 | 1.00 | 108.60 | A |
| ATOM | 2366 | C | LYS | A | 296 | −34.900 | −21.226 | 51.777 | 1.00 | 63.22 | A |
| ATOM | 2367 | O | LYS | A | 296 | −34.668 | −21.271 | 52.985 | 1.00 | 63.22 | A |
| ATOM | 2368 | N | ASN | A | 297 | −34.395 | −22.099 | 50.908 | 1.00 | 51.25 | A |
| ATOM | 2369 | CA | ASN | A | 297 | −33.547 | −23.211 | 51.328 | 1.00 | 51.25 | A |
| ATOM | 2370 | CB | ASN | A | 297 | −33.203 | −24.101 | 50.131 | 1.00 | 56.65 | A |
| ATOM | 2371 | CG | ASN | A | 297 | −32.561 | −25.413 | 50.548 | 1.00 | 56.65 | A |
| ATOM | 2372 | OD1 | ASN | A | 297 | −31.487 | −25.430 | 51.152 | 1.00 | 56.65 | A |
| ATOM | 2373 | ND2 | ASN | A | 297 | −33.224 | −26.520 | 50.234 | 1.00 | 56.65 | A |
| ATOM | 2374 | C | ASN | A | 297 | −32.263 | −22.800 | 52.034 | 1.00 | 51.25 | A |
| ATOM | 2375 | O | ASN | A | 297 | −31.552 | −21.892 | 51.615 | 1.00 | 51.25 | A |
| ATOM | 2376 | N | THR | A | 298 | −31.964 | −23.503 | 53.112 | 1.00 | 54.05 | A |
| ATOM | 2377 | CA | THR | A | 298 | −30.785 | −23.223 | 53.903 | 1.00 | 54.05 | A |
| ATOM | 2378 | CB | THR | A | 298 | −30.769 | −24.124 | 55.141 | 1.00 | 72.09 | A |
| ATOM | 2379 | OG1 | THR | A | 298 | −29.439 | −24.194 | 55.658 | 1.00 | 72.09 | A |
| ATOM | 2380 | CG2 | THR | A | 298 | −31.259 | −25.512 | 54.788 | 1.00 | 72.09 | A |
| ATOM | 2381 | C | THR | A | 298 | −29.473 | −23.373 | 53.130 | 1.00 | 54.05 | A |
| ATOM | 2382 | O | THR | A | 298 | −28.467 | −22.760 | 53.478 | 1.00 | 54.05 | A |
| ATOM | 2383 | N | LEU | A | 299 | −29.483 | −24.175 | 52.074 | 1.00 | 59.52 | A |
| ATOM | 2384 | CA | LEU | A | 299 | −28.270 | −24.393 | 51.284 | 1.00 | 59.52 | A |
| ATOM | 2385 | CB | LEU | A | 299 | −28.146 | −25.868 | 50.903 | 1.00 | 52.64 | A |
| ATOM | 2386 | CG | LEU | A | 299 | −27.918 | −26.808 | 52.083 | 1.00 | 52.64 | A |
| ATOM | 2387 | CD1 | LEU | A | 299 | −27.938 | −28.238 | 51.604 | 1.00 | 52.64 | A |
| ATOM | 2388 | CD2 | LEU | A | 299 | −26.581 | −26.475 | 52.741 | 1.00 | 52.64 | A |
| ATOM | 2389 | C | LEU | A | 299 | −28.185 | −23.551 | 50.023 | 1.00 | 59.52 | A |
| ATOM | 2390 | O | LEU | A | 299 | −27.264 | −23.719 | 49.224 | 1.00 | 59.52 | A |
| ATOM | 2391 | N | TRP | A | 300 | −29.141 | −22.646 | 49.853 | 1.00 | 54.96 | A |
| ATOM | 2392 | CA | TRP | A | 300 | −29.172 | −21.780 | 48.680 | 1.00 | 54.96 | A |
| ATOM | 2393 | CB | TRP | A | 300 | −30.216 | −20.679 | 48.867 | 1.00 | 57.00 | A |
| ATOM | 2394 | CG | TRP | A | 300 | −30.335 | −19.781 | 47.686 | 1.00 | 57.00 | A |
| ATOM | 2395 | CD2 | TRP | A | 300 | −30.692 | −20.169 | 46.358 | 1.00 | 57.00 | A |
| ATOM | 2396 | CE2 | TRP | A | 300 | −30.675 | −18.998 | 45.562 | 1.00 | 57.00 | A |
| ATOM | 2397 | CE3 | TRP | A | 300 | −31.026 | −21.393 | 45.760 | 1.00 | 57.00 | A |
| ATOM | 2398 | CD1 | TRP | A | 300 | −30.120 | −18.425 | 47.649 | 1.00 | 57.00 | A |
| ATOM | 2399 | NE1 | TRP | A | 300 | −30.324 | −17.950 | 46.374 | 1.00 | 57.00 | A |
| ATOM | 2400 | CZ2 | TRP | A | 300 | −30.980 | −19.017 | 44.200 | 1.00 | 57.00 | A |
| ATOM | 2401 | CZ3 | TRP | A | 300 | −31.330 | −21.412 | 44.407 | 1.00 | 57.00 | A |
| ATOM | 2402 | CH2 | TRP | A | 300 | −31.305 | −20.230 | 43.641 | 1.00 | 57.00 | A |
| ATOM | 2403 | C | TRP | A | 300 | −27.809 | −21.145 | 48.384 | 1.00 | 54.96 | A |
| ATOM | 2404 | O | TRP | A | 300 | −27.384 | −21.079 | 47.223 | 1.00 | 54.96 | A |
| ATOM | 2405 | N | LYS | A | 301 | −27.127 | −20.684 | 49.430 | 1.00 | 59.16 | A |
| ATOM | 2406 | CA | LYS | A | 301 | −25.834 | −20.049 | 49.251 | 1.00 | 59.16 | A |
| ATOM | 2407 | CB | LYS | A | 301 | −25.242 | −19.617 | 50.603 | 1.00 | 74.35 | A |
| ATOM | 2408 | CG | LYS | A | 301 | −26.108 | −18.609 | 51.370 | 1.00 | 74.35 | A |
| ATOM | 2409 | CD | LYS | A | 301 | −26.577 | −17.447 | 50.475 | 1.00 | 74.35 | A |
| ATOM | 2410 | CE | LYS | A | 301 | −27.763 | −16.691 | 51.082 | 1.00 | 74.35 | A |
| ATOM | 2411 | NZ | LYS | A | 301 | −28.476 | −15.846 | 50.083 | 1.00 | 74.35 | A |
| ATOM | 2412 | C | LYS | A | 301 | −24.879 | −20.971 | 48.519 | 1.00 | 59.16 | A |
| ATOM | 2413 | O | LYS | A | 301 | −24.084 | −20.522 | 47.691 | 1.00 | 59.16 | A |
| ATOM | 2414 | N | ASP | A | 302 | −24.960 | −22.263 | 48.806 | 1.00 | 46.62 | A |
| ATOM | 2415 | CA | ASP | A | 302 | −24.081 | −23.210 | 48.143 | 1.00 | 46.62 | A |
| ATOM | 2416 | CB | ASP | A | 302 | −24.075 | −24.548 | 48.879 | 1.00 | 86.65 | A |
| ATOM | 2417 | CG | ASP | A | 302 | −23.332 | −24.477 | 50.196 | 1.00 | 86.65 | A |
| ATOM | 2418 | OD1 | ASP | A | 302 | −22.186 | −23.979 | 50.197 | 1.00 | 86.65 | A |
| ATOM | 2419 | OD2 | ASP | A | 302 | −23.855 | −24.920 | 51.225 | 1.00 | 86.65 | A |
| ATOM | 2420 | C | ASP | A | 302 | −24.495 | −23.406 | 46.695 | 1.00 | 46.62 | A |
| ATOM | 2421 | O | ASP | A | 302 | −23.641 | −23.572 | 45.820 | 1.00 | 46.62 | A |
| ATOM | 2422 | N | VAL | A | 303 | −25.803 | −23.384 | 46.440 | 1.00 | 37.38 | A |
| ATOM | 2423 | CA | VAL | A | 303 | −26.298 | −23.543 | 45.084 | 1.00 | 37.38 | A |
| ATOM | 2424 | CB | VAL | A | 303 | −27.820 | −23.507 | 45.035 | 1.00 | 36.27 | A |
| ATOM | 2425 | CG1 | VAL | A | 303 | −28.291 | −23.514 | 43.600 | 1.00 | 25.58 | A |
| ATOM | 2426 | CG2 | VAL | A | 303 | −28.381 | −24.700 | 45.766 | 1.00 | 25.58 | A |
| ATOM | 2427 | C | VAL | A | 303 | −25.744 | −22.398 | 44.260 | 1.00 | 37.38 | A |
| ATOM | 2428 | O | VAL | A | 303 | −25.133 | −22.625 | 43.219 | 1.00 | 37.38 | A |
| ATOM | 2429 | N | LYS | A | 304 | −25.938 | −21.169 | 44.738 | 1.00 | 52.76 | A |
| ATOM | 2430 | CA | LYS | A | 304 | −25.443 | −19.984 | 44.038 | 1.00 | 52.76 | A |
| ATOM | 2431 | CB | LYS | A | 304 | −25.599 | −18.736 | 44.904 | 1.00 | 85.84 | A |
| ATOM | 2432 | CG | LYS | A | 304 | −26.951 | −18.068 | 44.857 | 1.00 | 67.76 | A |
| ATOM | 2433 | CD | LYS | A | 304 | −26.948 | −16.863 | 45.782 | 1.00 | 67.76 | A |
| ATOM | 2434 | CE | LYS | A | 304 | −28.257 | −16.110 | 45.713 | 1.00 | 67.76 | A |
| ATOM | 2435 | NZ | LYS | A | 304 | −28.322 | −15.027 | 46.722 | 1.00 | 67.76 | A |
| ATOM | 2436 | C | LYS | A | 304 | −23.974 | −20.135 | 43.692 | 1.00 | 52.76 | A |
| ATOM | 2437 | O | LYS | A | 304 | −23.546 | −19.869 | 42.572 | 1.00 | 52.76 | A |
| ATOM | 2438 | N | ALA | A | 305 | −23.201 | −20.561 | 44.671 | 1.00 | 54.43 | A |
| ATOM | 2439 | CA | ALA | A | 305 | −21.772 | −20.731 | 44.492 | 1.00 | 54.43 | A |
| ATOM | 2440 | CB | ALA | A | 305 | −21.126 | −20.937 | 45.852 | 1.00 | 17.97 | A |
| ATOM | 2441 | C | ALA | A | 305 | −21.365 | −21.857 | 43.539 | 1.00 | 54.43 | A |
| ATOM | 2442 | O | ALA | A | 305 | −20.578 | −21.634 | 42.617 | 1.00 | 54.43 | A |
| ATOM | 2443 | N | TYR | A | 306 | −21.891 | −23.060 | 43.761 | 1.00 | 43.71 | A |
| ATOM | 2444 | CA | TYR | A | 306 | −21.545 | −24.197 | 42.919 | 1.00 | 43.71 | A |
| ATOM | 2445 | CB | TYR | A | 306 | −21.985 | −25.503 | 43.583 | 1.00 | 53.74 | A |
| ATOM | 2446 | CG | TYR | A | 306 | −21.240 | −25.809 | 44.853 | 1.00 | 48.43 | A |
| ATOM | 2447 | CD1 | TYR | A | 306 | −21.844 | −26.530 | 45.882 | 1.00 | 48.43 | A |
| ATOM | 2448 | CE1 | TYR | A | 306 | −21.166 | −26.815 | 47.046 | 1.00 | 48.43 | A |
| ATOM | 2449 | CD2 | TYR | A | 306 | −19.930 | −25.384 | 45.028 | 1.00 | 48.43 | A |
| ATOM | 2450 | CE2 | TYR | A | 306 | −19.241 | −25.665 | 46.188 | 1.00 | 48.43 | A |
| ATOM | 2451 | CZ | TYR | A | 306 | −19.863 | −26.380 | 47.191 | 1.00 | 48.43 | A |
| ATOM | 2452 | OH | TYR | A | 306 | −19.170 | −26.673 | 48.339 | 1.00 | 48.43 | A |
| ATOM | 2453 | C | TYR | A | 306 | −22.124 | −24.121 | 41.523 | 1.00 | 43.71 | A |
| ATOM | 2454 | O | TYR | A | 306 | −21.403 | −24.283 | 40.553 | 1.00 | 43.71 | A |
| ATOM | 2455 | N | LEU | A | 307 | −23.422 | −23.876 | 41.420 | 1.00 | 36.14 | A |
| ATOM | 2456 | CA | LEU | A | 307 | −24.076 | −23.814 | 40.123 | 1.00 | 36.14 | A |
| ATOM | 2457 | CB | LEU | A | 307 | −25.566 | −24.127 | 40.264 | 1.00 | 56.60 | A |
| ATOM | 2458 | CG | LEU | A | 307 | −26.403 | −23.923 | 39.003 | 1.00 | 24.73 | A |
| ATOM | 2459 | CD1 | LEU | A | 307 | −25.917 | −24.828 | 37.879 | 1.00 | 24.73 | A |
| ATOM | 2460 | CD2 | LEU | A | 307 | −27.863 | −24.210 | 39.323 | 1.00 | 24.73 | A |
| ATOM | 2461 | C | LEU | A | 307 | −23.889 | −22.455 | 39.472 | 1.00 | 36.14 | A |
| ATOM | 2462 | O | LEU | A | 307 | −23.467 | −22.349 | 38.314 | 1.00 | 36.14 | A |
| ATOM | 2463 | N | GLY | A | 308 | −24.201 | −21.406 | 40.213 | 1.00 | 50.36 | A |
| ATOM | 2464 | CA | GLY | A | 308 | −24.044 | −20.077 | 39.664 | 1.00 | 50.36 | A |
| ATOM | 2465 | C | GLY | A | 308 | −22.611 | −19.795 | 39.252 | 1.00 | 50.36 | A |
| ATOM | 2466 | O | GLY | A | 308 | −22.363 | −19.297 | 38.154 | 1.00 | 50.36 | A |
| ATOM | 2467 | N | GLY | A | 309 | −21.663 | −20.115 | 40.126 | 1.00 | 39.03 | A |
| ATOM | 2468 | CA | GLY | A | 309 | −20.272 | −19.866 | 39.812 | 1.00 | 39.03 | A |
| ATOM | 2469 | C | GLY | A | 309 | −19.792 | −20.559 | 38.551 | 1.00 | 39.03 | A |
| ATOM | 2470 | O | GLY | A | 309 | −19.169 | −19.935 | 37.678 | 1.00 | 39.03 | A |
| ATOM | 2471 | N | PHE | A | 310 | −20.087 | −21.854 | 38.459 | 1.00 | 42.20 | A |
| ATOM | 2472 | CA | PHE | A | 310 | −19.679 | −22.664 | 37.327 | 1.00 | 42.20 | A |
| ATOM | 2473 | CB | PHE | A | 310 | −20.207 | −24.081 | 37.500 | 1.00 | 39.00 | A |
| ATOM | 2474 | CG | PHE | A | 310 | −19.631 | −25.067 | 36.526 | 1.00 | 34.83 | A |
| ATOM | 2475 | CD1 | PHE | A | 310 | −18.262 | −25.320 | 36.496 | 1.00 | 34.83 | A |
| ATOM | 2476 | CD2 | PHE | A | 310 | −20.459 | −25.762 | 35.652 | 1.00 | 34.83 | A |
| ATOM | 2477 | CE1 | PHE | A | 310 | −17.731 | −26.254 | 35.608 | 1.00 | 34.83 | A |
| ATOM | 2478 | CE2 | PHE | A | 310 | −19.937 | −26.694 | 34.764 | 1.00 | 34.83 | A |
| ATOM | 2479 | CZ | PHE | A | 310 | −18.571 | −26.944 | 34.739 | 1.00 | 34.83 | A |
| ATOM | 2480 | C | PHE | A | 310 | −20.241 | −22.064 | 36.050 | 1.00 | 42.20 | A |
| ATOM | 2481 | O | PHE | A | 310 | −19.532 | −21.930 | 35.036 | 1.00 | 42.20 | A |
| ATOM | 2482 | N | GLU | A | 311 | −21.517 | −21.690 | 36.115 | 1.00 | 35.00 | A |
| ATOM | 2483 | CA | GLU | A | 311 | −22.199 | −21.131 | 34.963 | 1.00 | 35.00 | A |
| ATOM | 2484 | CB | GLU | A | 311 | −23.704 | −21.094 | 35.205 | 1.00 | 91.84 | A |
| ATOM | 2485 | CG | GLU | A | 311 | −24.508 | −21.061 | 33.923 | 1.00 | 76.07 | A |
| ATOM | 2486 | CD | GLU | A | 311 | −25.981 | −20.843 | 34.159 | 1.00 | 76.07 | A |
| ATOM | 2487 | OE1 | GLU | A | 311 | −26.532 | −21.406 | 35.126 | 1.00 | 76.07 | A |
| ATOM | 2488 | OE2 | GLU | A | 311 | −26.598 | −20.115 | 33.364 | 1.00 | 76.07 | A |
| ATOM | 2489 | C | GLU | A | 311 | −21.699 | −19.734 | 34.639 | 1.00 | 35.00 | A |
| ATOM | 2490 | O | GLU | A | 311 | −21.628 | −19.341 | 33.482 | 1.00 | 35.00 | A |
| ATOM | 2491 | N | TYR | A | 312 | −21.333 | −18.982 | 35.661 | 1.00 | 42.68 | A |
| ATOM | 2492 | CA | TYR | A | 312 | −20.863 | −17.631 | 35.438 | 1.00 | 42.68 | A |
| ATOM | 2493 | CB | TYR | A | 312 | −20.792 | −16.872 | 36.751 | 1.00 | 48.53 | A |
| ATOM | 2494 | CG | TYR | A | 312 | −20.383 | −15.432 | 36.559 | 1.00 | 43.23 | A |
| ATOM | 2495 | CD1 | TYR | A | 312 | −21.277 | −14.500 | 36.027 | 1.00 | 43.23 | A |
| ATOM | 2496 | CE1 | TYR | A | 312 | −20.904 | −13.172 | 35.818 | 1.00 | 43.23 | A |
| ATOM | 2497 | CD2 | TYR | A | 312 | −19.092 | −15.000 | 36.883 | 1.00 | 43.23 | A |
| ATOM | 2498 | CE2 | TYR | A | 312 | −18.707 | −13.670 | 36.675 | 1.00 | 43.23 | A |
| ATOM | 2499 | CZ | TYR | A | 312 | −19.622 | −12.763 | 36.137 | 1.00 | 43.23 | A |
| ATOM | 2500 | OH | TYR | A | 312 | −19.239 | −11.461 | 35.882 | 1.00 | 43.23 | A |
| ATOM | 2501 | C | TYR | A | 312 | −19.498 | −17.556 | 34.769 | 1.00 | 42.68 | A |
| ATOM | 2502 | O | TYR | A | 312 | −19.246 | −16.660 | 33.972 | 1.00 | 42.68 | A |
| ATOM | 2503 | N | MET | A | 313 | −18.600 | −18.474 | 35.097 | 1.00 | 43.73 | A |
| ATOM | 2504 | CA | MET | A | 313 | −17.274 | −18.410 | 34.505 | 1.00 | 43.73 | A |
| ATOM | 2505 | CB | MET | A | 313 | −16.494 | −17.272 | 35.146 | 1.00 | 54.39 | A |
| ATOM | 2506 | CG | MET | A | 313 | −15.131 | −17.073 | 34.555 | 1.00 | 54.24 | A |
| ATOM | 2507 | SD | MET | A | 313 | −14.199 | −15.773 | 35.398 | 1.00 | 54.24 | A |
| ATOM | 2508 | CE | MET | A | 313 | −15.293 | −14.432 | 35.214 | 1.00 | 54.24 | A |
| ATOM | 2509 | C | MET | A | 313 | −16.480 | −19.703 | 34.636 | 1.00 | 43.73 | A |
| ATOM | 2510 | O | MET | A | 313 | −15.703 | −20.060 | 33.746 | 1.00 | 43.73 | A |
| ATOM | 2511 | N | GLY | A | 314 | −16.677 | −20.401 | 35.749 | 1.00 | 34.56 | A |
| ATOM | 2512 | CA | GLY | A | 314 | −15.957 | −21.636 | 35.979 | 1.00 | 34.56 | A |
| ATOM | 2513 | C | GLY | A | 314 | −15.888 | −22.576 | 34.792 | 1.00 | 34.56 | A |
| ATOM | 2514 | O | GLY | A | 314 | −14.806 | −23.009 | 34.380 | 1.00 | 34.56 | A |
| ATOM | 2515 | N | CYS | A | 315 | −17.050 | −22.897 | 34.243 | 1.00 | 41.13 | A |
| ATOM | 2516 | CA | CYS | A | 315 | −17.107 | −23.802 | 33.124 | 1.00 | 41.13 | A |
| ATOM | 2517 | CB | CYS | A | 315 | −18.556 | −24.043 | 32.731 | 1.00 | 47.54 | A |
| ATOM | 2518 | SG | CYS | A | 315 | −18.746 | −25.461 | 31.664 | 1.00 | 52.91 | A |
| ATOM | 2519 | C | CYS | A | 315 | 16.319 | −23.304 | 31.916 | 1.00 | 41.13 | A |
| ATOM | 2520 | O | CYS | A | 315 | −15.495 | −24.037 | 31.372 | 1.00 | 41.13 | A |
| ATOM | 2521 | N | SER | A | 316 | −16.562 | −22.071 | 31.487 | 1.00 | 42.04 | A |
| ATOM | 2522 | CA | SER | A | 316 | −15.851 | −21.540 | 30.325 | 1.00 | 42.04 | A |
| ATOM | 2523 | CB | SER | A | 316 | −16.252 | −20.101 | 30.063 | 1.00 | 37.42 | A |
| ATOM | 2524 | OG | SER | A | 316 | −15.795 | −19.283 | 31.112 | 1.00 | 42.92 | A |
| ATOM | 2525 | C | SER | A | 316 | −14.336 | −21.604 | 30.485 | 1.00 | 42.04 | A |
| ATOM | 2526 | O | SER | A | 316 | −13.609 | −21.676 | 29.500 | 1.00 | 42.04 | A |
| ATOM | 2527 | N | LEU | A | 317 | −13.853 | −21.558 | 31.720 | 1.00 | 39.03 | A |
| ATOM | 2528 | CA | LEU | A | 317 | −12.424 | −21.651 | 31.959 | 1.00 | 39.03 | A |
| ATOM | 2529 | CB | LEU | A | 317 | −12.074 | −21.060 | 33.320 | 1.00 | 54.31 | A |
| ATOM | 2530 | CG | LEU | A | 317 | −12.189 | −19.537 | 33.427 | 1.00 | 46.82 | A |
| ATOM | 2531 | CD1 | LEU | A | 317 | −11.960 | −19.098 | 34.864 | 1.00 | 46.82 | A |
| ATOM | 2532 | CD2 | LEU | A | 317 | −11.162 | −18.888 | 32.516 | 1.00 | 46.82 | A |
| ATOM | 2533 | C | LEU | A | 317 | −11.990 | −23.117 | 31.909 | 1.00 | 39.03 | A |
| ATOM | 2534 | O | LEU | A | 317 | −10.985 | −23.472 | 31.287 | 1.00 | 39.03 | A |
| ATOM | 2535 | N | ALA | A | 318 | −12.760 | −23.974 | 32.563 | 1.00 | 33.35 | A |
| ATOM | 2536 | CA | ALA | A | 318 | −12.450 | −25.389 | 32.595 | 1.00 | 33.35 | A |
| ATOM | 2537 | CB | ALA | A | 318 | −13.452 | −26.103 | 33.483 | 1.00 | 23.27 | A |
| ATOM | 2538 | C | ALA | A | 318 | −12.424 | −26.028 | 31.207 | 1.00 | 33.35 | A |
| ATOM | 2539 | O | ALA | A | 318 | −11.487 | −26.758 | 30.876 | 1.00 | 33.35 | A |
| ATOM | 2540 | N | ILE | A | 319 | −13.441 | −25.746 | 30.394 | 1.00 | 43.03 | A |
| ATOM | 2541 | CA | ILE | A | 319 | −13.520 | −26.341 | 29.066 | 1.00 | 43.03 | A |
| ATOM | 2542 | CB | ILE | A | 319 | −14.646 | −25.709 | 28.207 | 1.00 | 53.37 | A |
| ATOM | 2543 | CG2 | ILE | A | 319 | −14.247 | −24.324 | 27.748 | 1.00 | 27.93 | A |
| ATOM | 2544 | CG1 | ILE | A | 319 | −14.893 | −26.572 | 26.971 | 1.00 | 27.93 | A |
| ATOM | 2545 | CD1 | ILE | A | 319 | −16.093 | −26.150 | 26.174 | 1.00 | 27.93 | A |
| ATOM | 2546 | C | ILE | A | 319 | −12.209 | −26.249 | 28.307 | 1.00 | 43.03 | A |
| ATOM | 2547 | O | ILE | A | 319 | −11.883 | −27.132 | 27.523 | 1.00 | 43.03 | A |
| ATOM | 2548 | N | LEU | A | 320 | −11.448 | −25.190 | 28.553 | 1.00 | 43.89 | A |
| ATOM | 2549 | CA | LEU | A | 320 | −10.170 | −25.011 | 27.874 | 1.00 | 43.89 | A |
| ATOM | 2550 | CB | LEU | A | 320 | −9.675 | −23.570 | 28.044 | 1.00 | 66.07 | A |
| ATOM | 2551 | CG | LEU | A | 320 | −10.558 | −22.486 | 27.393 | 1.00 | 43.91 | A |
| ATOM | 2552 | CD1 | LEU | A | 320 | −9.946 | −21.132 | 27.617 | 1.00 | 43.91 | A |
| ATOM | 2553 | CD2 | LEU | A | 320 | −10.717 | −22.740 | 25.899 | 1.00 | 43.91 | A |
| ATOM | 2554 | C | LEU | A | 320 | −9.108 | −26.009 | 28.343 | 1.00 | 43.89 | A |
| ATOM | 2555 | O | LEU | A | 320 | −8.228 | −26.399 | 27.572 | 1.00 | 43.89 | A |
| ATOM | 2556 | N | ASP | A | 321 | −9.185 | −26.429 | 29.602 | 1.00 | 48.56 | A |
| ATOM | 2557 | CA | ASP | A | 321 | −8.235 | −27.410 | 30.109 | 1.00 | 48.56 | A |
| ATOM | 2558 | CB | ASP | A | 321 | −8.297 | −27.495 | 31.635 | 1.00 | 89.28 | A |
| ATOM | 2559 | CG | ASP | A | 321 | −71.18 | −26.266 | 32.315 | 1.00 | 68.28 | A |
| ATOM | 2560 | OD1 | ASP | A | 321 | −6.642 | −25.801 | 31.875 | 1.00 | 68.28 | A |
| ATOM | 2561 | OD2 | ASP | A | 321 | −8.329 | −25.777 | 33.300 | 1.00 | 68.28 | A |
| ATOM | 2562 | C | ASP | A | 321 | −8.549 | −28.781 | 29.501 | 1.00 | 48.56 | A |
| ATOM | 2563 | O | ASP | A | 321 | −7.639 | −29.553 | 29.178 | 1.00 | 48.56 | A |
| ATOM | 2564 | N | TYR | A | 322 | −9.836 | −29.083 | 29.346 | 1.00 | 47.67 | A |
| ATOM | 2565 | CA | TYR | A | 322 | −10.231 | −30.354 | 28.761 | 1.00 | 47.67 | A |
| ATOM | 2566 | CB | TYR | A | 322 | −11.755 | −30.494 | 28.714 | 1.00 | 51.44 | A |
| ATOM | 2567 | CG | TYR | A | 322 | −12.410 | −30.816 | 30.047 | 1.00 | 42.15 | A |
| ATOM | 2568 | CD1 | TYR | A | 322 | −12.496 | −29.861 | 31.057 | 1.00 | 42.15 | A |
| ATOM | 2569 | CE1 | TYR | A | 322 | −13.133 | −30.139 | 32.264 | 1.00 | 42.15 | A |
| ATOM | 2570 | CD2 | TYR | A | 322 | −12.975 | −32.066 | 30.287 | 1.00 | 42.15 | A |
| ATOM | 2571 | CE2 | TYR | A | 322 | −13.612 | −32.350 | 31.493 | 1.00 | 42.15 | A |
| ATOM | 2572 | CZ | TYR | A | 322 | −13.688 | −31.378 | 32.470 | 1.00 | 42.15 | A |
| ATOM | 2573 | OH | TYR | A | 322 | −14.343 | −31.627 | 33.649 | 1.00 | 42.15 | A |
| ATOM | 2574 | C | TYR | A | 322 | −9.683 | −30.422 | 27.346 | 1.00 | 47.67 | A |
| ATOM | 2575 | O | TYR | A | 322 | −9.065 | −31.419 | 26.944 | 1.00 | 47.67 | A |
| ATOM | 2576 | N | GLN | A | 323 | −9.914 | −29.348 | 26.595 | 1.00 | 61.50 | A |
| ATOM | 2577 | CA | GLN | A | 323 | −9.468 | −29.271 | 25.223 | 1.00 | 61.50 | A |
| ATOM | 2578 | CB | GLN | A | 323 | −9.754 | −27.895 | 24.658 | 1.00 | 74.81 | A |
| ATOM | 2579 | CG | GLN | A | 323 | −9.314 | −27.764 | 23.238 | 1.00 | 49.20 | A |
| ATOM | 2580 | CD | GLN | A | 323 | −9.699 | −26.442 | 22.642 | 1.00 | 49.20 | A |
| ATOM | 2581 | OE1 | GLN | A | 323 | −9.427 | −25.380 | 23.223 | 1.00 | 49.20 | A |
| ATOM | 2582 | NE2 | GLN | A | 323 | −10.339 | −26.484 | 21.470 | 1.00 | 49.20 | A |
| ATOM | 2583 | C | GLN | A | 323 | −7.987 | −29.589 | 25.102 | 1.00 | 61.50 | A |
| ATOM | 2584 | O | GLN | A | 323 | −7.595 | −30.368 | 24.237 | 1.00 | 61.50 | A |
| ATOM | 2585 | N | LYS | A | 324 | −7.167 | −28.996 | 25.966 | 1.00 | 53.33 | A |
| ATOM | 2586 | CA | LYS | A | 324 | −5.723 | −29.248 | 25.945 | 1.00 | 53.33 | A |
| ATOM | 2587 | CB | LYS | A | 324 | −5.010 | −28.495 | 27.063 | 1.00 | 77.76 | A |
| ATOM | 2588 | CG | LYS | A | 324 | −4.790 | −27.041 | 26.807 | 1.00 | 67.19 | A |
| ATOM | 2589 | CD | LYS | A | 324 | −3.855 | −26.464 | 27.848 | 1.00 | 67.19 | A |
| ATOM | 2590 | CE | LYS | A | 324 | −3.596 | −24.973 | 27.594 | 1.00 | 67.19 | A |
| ATOM | 2591 | NZ | LYS | A | 324 | −2.527 | −24.399 | 28.471 | 1.00 | 67.19 | A |
| ATOM | 2592 | C | LYS | A | 324 | −5.391 | −30.723 | 26.103 | 1.00 | 53.33 | A |
| ATOM | 2593 | O | LYS | A | 324 | −4.632 | −31.285 | 25.316 | 1.00 | 53.33 | A |
| ATOM | 2594 | N | MET | A | 325 | −5.951 | −31.350 | 27.131 | 1.00 | 54.40 | A |
| ATOM | 2595 | CA | MET | A | 325 | −5.678 | −32.753 | 27.384 | 1.00 | 54.40 | A |
| ATOM | 2596 | CB | MET | A | 325 | −6.090 | −33.108 | 28.804 | 1.00 | 68.42 | A |
| ATOM | 2597 | CG | MET | A | 325 | −5.290 | −32.354 | 29.839 | 1.00 | 56.98 | A |
| ATOM | 2598 | SD | MET | A | 325 | −5.713 | −32.721 | 31.544 | 1.00 | 56.98 | A |
| ATOM | 2599 | CE | MET | A | 325 | −6.876 | −31.468 | 31.891 | 1.00 | 56.98 | A |
| ATOM | 2600 | C | MET | A | 325 | −6.350 | −33.687 | 26.407 | 1.00 | 54.40 | A |
| ATOM | 2601 | O | MET | A | 325 | −6.006 | −34.851 | 26.339 | 1.00 | 54.40 | A |
| ATOM | 2602 | N | ALA | A | 326 | −7.303 | −33.187 | 25.639 | 1.00 | 51.00 | A |
| ATOM | 2603 | CA | ALA | A | 326 | −7.989 | −34.049 | 24.697 | 1.00 | 51.00 | A |
| ATOM | 2604 | CB | ALA | A | 326 | −9.467 | −33.653 | 24.611 | 1.00 | 32.02 | A |
| ATOM | 2605 | C | ALA | A | 326 | −7.357 | −34.006 | 23.315 | 1.00 | 51.00 | A |
| ATOM | 2606 | O | ALA | A | 326 | −7.475 | −34.962 | 22.552 | 1.00 | 51.00 | A |
| ATOM | 2607 | N | LYS | A | 327 | −6.677 | −32.903 | 23.009 | 1.00 | 88.79 | A |
| ATOM | 2608 | CA | LYS | A | 327 | −6.062 | −32.688 | 21.704 | 1.00 | 88.79 | A |
| ATOM | 2609 | CB | LYS | A | 327 | −4.759 | −31.907 | 21.838 | 1.00 | 167.55 | A |
| ATOM | 2610 | CG | LYS | A | 327 | −4.275 | −31.390 | 20.499 | 1.00 | 85.84 | A |
| ATOM | 2611 | CD | LYS | A | 327 | −3.066 | −30.491 | 20.632 | 1.00 | 85.84 | A |
| ATOM | 2612 | CE | LYS | A | 327 | −2.663 | −29.912 | 19.276 | 1.00 | 85.84 | A |
| ATOM | 2613 | NZ | LYS | A | 327 | −1.446 | −29.046 | 19.357 | 1.00 | 85.84 | A |
| ATOM | 2614 | C | LYS | A | 327 | −5.813 | −33.930 | 20.862 | 1.00 | 88.79 | A |
| ATOM | 2615 | O | LYS | A | 327 | −6.450 | −34.127 | 19.824 | 1.00 | 88.79 | A |
| ATOM | 2616 | N | GLY | A | 328 | −4.897 | −34.781 | 21.298 | 1.00 | 57.80 | A |
| ATOM | 2617 | CA | GLY | A | 328 | −4.626 | −35.963 | 20.506 | 1.00 | 57.80 | A |
| ATOM | 2618 | C | GLY | A | 328 | −5.488 | −37.196 | 20.725 | 1.00 | 57.80 | A |
| ATOM | 2619 | O | GLY | A | 328 | −5.633 | −38.004 | 19.815 | 1.00 | 57.80 | A |
| ATOM | 2620 | N | LYS | A | 329 | −6.087 | −37.346 | 21.902 | 1.00 | 54.27 | A |
| ATOM | 2621 | CA | LYS | A | 329 | −6.863 | −38.546 | 22.177 | 1.00 | 54.27 | A |
| ATOM | 2622 | CB | LYS | A | 329 | −6.694 | −38.925 | 23.648 | 1.00 | 187.60 | A |
| ATOM | 2623 | CG | LYS | A | 329 | −5.268 | −39.307 | 24.015 | 1.00 | 187.60 | A |
| ATOM | 2624 | CD | LYS | A | 329 | −5.240 | −40.070 | 25.321 | 1.00 | 187.60 | A |
| ATOM | 2625 | CE | LYS | A | 329 | −3.842 | −40.545 | 25.673 | 1.00 | 77.36 | A |
| ATOM | 2626 | NZ | LYS | A | 329 | −3.863 | −41.325 | 26.949 | 1.00 | 77.36 | A |
| ATOM | 2627 | C | LYS | A | 329 | −8.338 | −38.669 | 21.792 | 1.00 | 54.27 | A |
| ATOM | 2628 | O | LYS | A | 329 | −9.020 | −39.565 | 22.296 | 1.00 | 54.27 | A |
| ATOM | 2629 | N | ILE | A | 330 | −8.846 | −37.809 | 20.912 | 1.00 | 42.16 | A |
| ATOM | 2630 | CA | ILE | A | 330 | −10.256 | −37.946 | 20.500 | 1.00 | 42.16 | A |
| ATOM | 2631 | CB | ILE | A | 330 | −11.190 | −36.972 | 21.277 | 1.00 | 49.41 | A |
| ATOM | 2632 | CG2 | ILE | A | 330 | −10.995 | −37.144 | 22.783 | 1.00 | 50.33 | A |
| ATOM | 2633 | CG1 | ILE | A | 330 | −10.910 | −35.527 | 20.870 | 1.00 | 50.33 | A |
| ATOM | 2634 | CD1 | ILE | A | 330 | −12.004 | −34.572 | 21.269 | 1.00 | 50.33 | A |
| ATOM | 2635 | C | ILE | A | 330 | −10.418 | −37.707 | 19.006 | 1.00 | 42.16 | A |
| ATOM | 2636 | O | ILE | A | 330 | −9.482 | −37.268 | 18.352 | 1.00 | 42.16 | A |
| ATOM | 2637 | N | PRO | A | 331 | −11.608 | −37.983 | 18.449 | 1.00 | 42.62 | A |
| ATOM | 2638 | CD | PRO | A | 331 | −12.838 | −38.443 | 19.105 | 1.00 | 26.64 | A |
| ATOM | 2639 | CA | PRO | A | 331 | −11.848 | −37.782 | 17.015 | 1.00 | 42.62 | A |
| ATOM | 2640 | CB | PRO | A | 331 | −13.361 | −37.881 | 16.899 | 1.00 | 170.62 | A |
| ATOM | 2641 | CG | PRO | A | 331 | −13.688 | −38.870 | 17.910 | 1.00 | 26.64 | A |
| ATOM | 2642 | C | PRO | A | 331 | −11.331 | −36.438 | 16.539 | 1.00 | 42.62 | A |
| ATOM | 2643 | O | PRO | A | 331 | −11.516 | −35.427 | 17.218 | 1.00 | 42.62 | A |
| ATOM | 2644 | N | LYS | A | 332 | −10.671 | −36.428 | 15.381 | 1.00 | 54.24 | A |
| ATOM | 2645 | CA | LYS | A | 332 | −10.138 | −35.194 | 14.820 | 1.00 | 54.86 | A |
| ATOM | 2646 | CB | LYS | A | 332 | −9.168 | −35.511 | 13.688 | 1.00 | 119.49 | A |
| ATOM | 2647 | CG | LYS | A | 332 | −9.830 | −36.263 | 12.551 | 1.00 | 91.03 | A |
| ATOM | 2648 | CD | LYS | A | 332 | −9.038 | −36.138 | 11.267 | 1.00 | 91.03 | A |
| ATOM | 2649 | CE | LYS | A | 332 | −9.818 | −36.696 | 10.087 | 1.00 | 91.03 | A |
| ATOM | 2650 | NZ | LYS | A | 332 | −9.109 | −36.451 | 8.799 | 1.00 | 91.03 | A |
| ATOM | 2651 | C | LYS | A | 332 | −11.286 | −34.330 | 14.279 | 1.00 | 53.93 | A |
| ATOM | 2652 | O | LYS | A | 332 | −12.370 | −34.826 | 13.967 | 1.00 | 52.02 | A |
| ATOM | 2653 | N | ASP | A | 333 | −11.047 | −33.031 | 14.174 | 1.00 | 64.68 | A |
| ATOM | 2654 | CA | ASP | A | 333 | −12.061 | −32.132 | 13.648 | 1.00 | 64.14 | A |
| ATOM | 2655 | CB | ASP | A | 333 | −11.564 | −30.686 | 13.706 | 1.00 | 142.70 | A |
| ATOM | 2656 | CG | ASP | A | 333 | −11.903 | −30.007 | 15.015 | 1.00 | 110.29 | A |
| ATOM | 2657 | OD1 | ASP | A | 333 | −11.850 | −30.670 | 16.068 | 1.00 | 110.29 | A |
| ATOM | 2658 | OD2 | ASP | A | 333 | −12.214 | −28.803 | 14.996 | 1.00 | 110.29 | A |
| ATOM | 2659 | C | ASP | A | 333 | −12.367 | −32.518 | 12.208 | 1.00 | 67.99 | A |
| ATOM | 2660 | O | ASP | A | 333 | −11.474 | −32.894 | 11.443 | 1.00 | 67.64 | A |
| ATOM | 2661 | N | THR | A | 334 | −13.637 | −32.435 | 11.842 | 1.00 | 32.26 | A |
| ATOM | 2662 | CA | THR | A | 334 | −14.050 | −32.761 | 10.484 | 1.00 | 31.98 | A |
| ATOM | 2663 | CB | THR | A | 334 | −14.640 | −34.167 | 10.421 | 1.00 | 64.41 | A |
| ATOM | 2664 | OG1 | THR | A | 334 | −13.572 | −35.113 | 10.548 | 1.00 | 50.31 | A |
| ATOM | 2665 | CG2 | THR | A | 334 | −15.394 | −34.391 | 9.111 | 1.00 | 50.31 | A |
| ATOM | 2666 | C | THR | A | 334 | −15.062 | −31.755 | 9.951 | 1.00 | 38.42 | A |
| ATOM | 2667 | O | THR | A | 334 | −16.150 | −31.591 | 10.507 | 1.00 | 35.78 | A |
| ATOM | 2668 | N | THR | A | 335 | −14.689 | −31.085 | 8.860 | 1.00 | 52.82 | A |
| ATOM | 2669 | CA | THR | A | 335 | −15.545 | −30.077 | 8.254 | 1.00 | 52.61 | A |
| ATOM | 2670 | CB | THR | A | 335 | −14.755 | −29.200 | 7.319 | 1.00 | 60.38 | A |
| ATOM | 2671 | OG1 | THR | A | 335 | −13.542 | −28.801 | 7.970 | 1.00 | 49.74 | A |
| ATOM | 2672 | CG2 | THR | A | 335 | −15.569 | −27.978 | 6.961 | 1.00 | 49.74 | A |
| ATOM | 2673 | C | THR | A | 335 | −16.708 | −30.672 | 7.496 | 1.00 | 53.47 | A |
| ATOM | 2674 | O | THR | A | 335 | −16.548 | −31.582 | 6.708 | 1.00 | 54.82 | A |
| ATOM | 2675 | N | PRO | A | 336 | −17.906 | −30.146 | 7.719 | 1.00 | 48.72 | A |
| ATOM | 2676 | CD | PRO | A | 336 | −18.260 | −29.063 | 8.653 | 1.00 | 40.71 | A |
| ATOM | 2677 | CA | PRO | A | 336 | −19.098 | −30.650 | 7.048 | 1.00 | 48.30 | A |
| ATOM | 2678 | CB | PRO | A | 336 | −20.205 | −30.215 | 7.989 | 1.00 | 49.57 | A |
| ATOM | 2679 | CG | PRO | A | 336 | −19.740 | −28.841 | 8.359 | 1.00 | 40.71 | A |
| ATOM | 2680 | C | PRO | A | 336 | −19.319 | −30.100 | 5.645 | 1.00 | 48.34 | A |
| ATOM | 2681 | O | PRO | A | 336 | −18.612 | −29.202 | 5.193 | 1.00 | 48.97 | A |
| ATOM | 2682 | N | THR | A | 337 | −20.331 | −30.659 | 4.989 | 1.00 | 47.72 | A |
| ATOM | 2683 | CA | THR | A | 337 | −20.751 | −30.275 | 3.655 | 1.00 | 51.18 | A |
| ATOM | 2684 | CB | THR | A | 337 | −20.890 | −31.494 | 2.754 | 1.00 | 111.85 | A |
| ATOM | 2685 | OG1 | THR | A | 337 | −19.593 | −32.000 | 2.442 | 1.00 | 62.74 | A |
| ATOM | 2686 | CG2 | THR | A | 337 | −21.622 | −31.139 | 1.483 | 1.00 | 62.74 | A |
| ATOM | 2687 | C | THR | A | 337 | −22.130 | −29.694 | 3.871 | 1.00 | 58.75 | A |
| ATOM | 2688 | O | THR | A | 337 | −22.953 | −30.314 | 4.535 | 1.00 | 50.13 | A |
| ATOM | 2689 | N | VAL | A | 338 | −22.397 | −28.517 | 3.325 | 1.00 | 61.11 | A |
| ATOM | 2690 | CA | VAL | A | 338 | −23.705 | −27.923 | 3.529 | 1.00 | 67.97 | A |
| ATOM | 2691 | CB | VAL | A | 338 | −23.602 | −26.576 | 4.283 | 1.00 | 41.43 | A |
| ATOM | 2692 | CG1 | VAL | A | 338 | −24.988 | −26.094 | 4.702 | 1.00 | 41.05 | A |
| ATOM | 2693 | CG2 | VAL | A | 338 | −22.686 | −26.724 | 5.481 | 1.00 | 41.05 | A |
| ATOM | 2694 | C | VAL | A | 338 | −24.418 | −27.694 | 2.217 | 1.00 | 72.44 | A |
| ATOM | 2695 | O | VAL | A | 338 | −23.792 | −27.421 | 1.187 | 1.00 | 72.27 | A |
| ATOM | 2696 | N | LYS | A | 339 | −25.736 | −27.825 | 2.269 | 1.00 | 44.06 | A |
| ATOM | 2697 | CA | LYS | A | 339 | −26.598 | −27.616 | 1.114 | 1.00 | 41.94 | A |
| ATOM | 2698 | CB | LYS | A | 339 | −26.729 | −28.909 | 0.311 | 1.00 | 175.42 | A |
| ATOM | 2699 | CG | LYS | A | 339 | −25.419 | −29.370 | −0.311 | 1.00 | 143.73 | A |
| ATOM | 2700 | CD | LYS | A | 339 | −25.329 | −30.883 | −0.385 | 1.00 | 143.73 | A |
| ATOM | 2701 | CE | LYS | A | 339 | −23.948 | −31.323 | −0.851 | 1.00 | 143.73 | A |
| ATOM | 2702 | NZ | LYS | A | 339 | −23.758 | −32.793 | −0.716 | 1.00 | 143.73 | A |
| ATOM | 2703 | C | LYS | A | 339 | −27.952 | −27.187 | 1.657 | 1.00 | 50.93 | A |
| ATOM | 2704 | O | LYS | A | 339 | −28.451 | −27.788 | 2.605 | 1.00 | 43.59 | A |
| ATOM | 2705 | N | VAL | A | 340 | −28.525 | −26.132 | 1.088 | 1.00 | 61.42 | A |
| ATOM | 2706 | CA | VAL | A | 340 | −29.830 | −25.654 | 1.531 | 1.00 | 58.85 | A |
| ATOM | 2707 | CB | VAL | A | 340 | −29.849 | −24.137 | 1.754 | 1.00 | 60.62 | A |
| ATOM | 2708 | CG1 | VAL | A | 340 | −31.237 | −23.712 | 2.201 | 1.00 | 46.08 | A |
| ATOM | 2709 | CG2 | VAL | A | 340 | −28.810 | −23.738 | 2.788 | 1.00 | 46.08 | A |
| ATOM | 2710 | C | VAL | A | 340 | −30.832 | −25.964 | 0.433 | 1.00 | 61.42 | A |
| ATOM | 2711 | O | VAL | A | 340 | −30.653 | −25.539 | −0.693 | 1.00 | 58.27 | A |
| ATOM | 2712 | N | THR | A | 341 | −31.893 | −26.692 | 0.752 | 1.00 | 64.19 | A |
| ATOM | 2713 | CA | THR | A | 341 | −32.886 | −27.037 | −0.257 | 1.00 | 60.09 | A |
| ATOM | 2714 | CB | THR | A | 341 | −33.136 | −28.535 | −0.285 | 1.00 | 79.90 | A |
| ATOM | 2715 | OG1 | THR | A | 341 | −31.916 | −29.215 | −0.600 | 1.00 | 61.26 | A |
| ATOM | 2716 | CG2 | THR | A | 341 | −34.175 | −28.865 | −1.330 | 1.00 | 61.26 | A |
| ATOM | 2717 | C | THR | A | 341 | −34.215 | −26.336 | −0.052 | 1.00 | 62.24 | A |
| ATOM | 2718 | O | THR | A | 341 | −34.651 | −26.121 | 1.078 | 1.00 | 63.84 | A |
| ATOM | 2719 | N | GLY | A | 342 | −34.860 | −25.977 | −1.155 | 1.00 | 114.98 | A |
| ATOM | 2720 | CA | GLY | A | 342 | −36.132 | −25.294 | −1.059 | 1.00 | 119.15 | A |
| ATOM | 2721 | C | GLY | A | 342 | −37.307 | −26.159 | −1.459 | 1.00 | 118.12 | A |
| ATOM | 2722 | O | GLY | A | 342 | −37.978 | −25.873 | −2.449 | 1.00 | 112.39 | A |
| ATOM | 2723 | N | ASN | A | 343 | −37.559 | −27.217 | −0.694 | 1.00 | 99.59 | A |
| ATOM | 2724 | CA | ASN | A | 343 | −38.678 | −28.118 | −0.975 | 1.00 | 106.00 | A |
| ATOM | 2725 | CB | ASN | A | 343 | −38.617 | −29.350 | −0.067 | 1.00 | 176.57 | A |
| ATOM | 2726 | CG | ASN | A | 343 | −37.275 | −30.048 | −0.117 | 1.00 | 156.37 | A |
| ATOM | 2727 | OD1 | ASN | A | 343 | −36.857 | −30.541 | −1.165 | 1.00 | 156.37 | A |
| ATOM | 2728 | ND2 | ASN | A | 343 | −36.589 | −30.091 | 1.021 | 1.00 | 156.37 | A |
| ATOM | 2729 | C | ASN | A | 343 | −39.967 | −27.361 | −0.699 | 1.00 | 104.06 | A |
| ATOM | 2730 | O | ASN | A | 343 | −39.960 | −26.139 | −0.569 | 1.00 | 92.69 | A |
| ATOM | 2731 | N | GLU | A | 344 | −41.075 | −28.086 | −0.611 | 1.00 | 174.22 | A |
| ATOM | 2732 | CA | GLU | A | 344 | −42.364 | −27.466 | −0.325 | 1.00 | 174.22 | A |
| ATOM | 2733 | CB | GLU | A | 344 | −42.891 | −26.728 | −1.560 | 1.00 | 138.88 | A |
| ATOM | 2734 | CG | GLU | A | 344 | −42.410 | −25.287 | −1.613 | 1.00 | 118.78 | A |
| ATOM | 2735 | CD | GLU | A | 344 | −42.761 | −24.574 | −2.900 | 1.00 | 118.78 | A |
| ATOM | 2736 | OE1 | GLU | A | 344 | −42.123 | −24.862 | −3.935 | 1.00 | 118.78 | A |
| ATOM | 2737 | OE2 | GLU | A | 344 | −43.674 | −23.723 | −2.874 | 1.00 | 118.78 | A |
| ATOM | 2738 | C | GLU | A | 344 | −43.364 | −28.497 | 0.176 | 1.00 | 174.22 | A |
| ATOM | 2739 | O | GLU | A | 344 | −43.840 | −29.345 | −0.580 | 1.00 | 174.22 | A |
| ATOM | 2740 | N | LEU | A | 345 | −43.673 | −28.403 | 1.469 | 1.00 | 200.84 | A |
| ATOM | 2741 | CA | LEU | A | 345 | −44.580 | −29.326 | 2.152 | 1.00 | 200.84 | A |
| ATOM | 2742 | CB | LEU | A | 345 | −44.381 | −29.210 | 3.675 | 1.00 | 176.20 | A |
| ATOM | 2743 | CG | LEU | A | 345 | −42.987 | −29.532 | 4.243 | 1.00 | 128.99 | A |
| ATOM | 2744 | CD1 | LEU | A | 345 | −43.002 | −29.411 | 5.762 | 1.00 | 128.99 | A |
| ATOM | 2745 | CD2 | LEU | A | 345 | −42.574 | −30.935 | 3.836 | 1.00 | 128.99 | A |
| ATOM | 2746 | C | LEU | A | 345 | −46.078 | −29.245 | 1.816 | 1.00 | 200.84 | A |
| ATOM | 2747 | O | LEU | A | 345 | −46.926 | −29.474 | 2.679 | 1.00 | 200.84 | A |
| ATOM | 2748 | N | GLU | A | 346 | −46.390 | −28.918 | 0.565 | 1.00 | 187.27 | A |
| ATOM | 2749 | CA | GLU | A | 346 | 47.769 | −28.859 | 0.066 | 1.00 | 187.27 | A |
| ATOM | 2750 | CB | GLU | A | 346 | 48.390 | −30.256 | 0.173 | 1.00 | 157.73 | A |
| ATOM | 2751 | CG | GLU | A | 346 | −47.424 | −31.356 | −0.261 | 1.00 | 143.16 | A |
| ATOM | 2752 | CD | GLU | A | 346 | −48.110 | −32.660 | −0.618 | 1.00 | 143.16 | A |
| ATOM | 2753 | OE1 | GLU | A | 346 | −48.855 | −32.682 | −1.622 | 1.00 | 143.16 | A |
| ATOM | 2754 | OE2 | GLU | A | 346 | −47.901 | −33.662 | 0.102 | 1.00 | 143.16 | A |
| ATOM | 2755 | C | GLU | A | 346 | −48.750 | −27.813 | 0.619 | 1.00 | 187.27 | A |
| ATOM | 2756 | O | GLU | A | 346 | −49.069 | −26.841 | −0.072 | 1.00 | 187.27 | A |
| ATOM | 2757 | N | ASP | A | 347 | −49.243 | −28.009 | 1.840 | 1.00 | 192.07 | A |
| ATOM | 2758 | CA | ASP | A | 347 | −50.204 | −27.066 | 2.418 | 1.00 | 192.07 | A |
| ATOM | 2759 | CB | ASP | A | 347 | −50.710 | −27.565 | 3.782 | 1.00 | 174.30 | A |
| ATOM | 2760 | CG | ASP | A | 347 | −49.764 | −27.234 | 4.920 | 1.00 | 174.30 | A |
| ATOM | 2761 | OD1 | ASP | A | 347 | −48.608 | −27.705 | 4.900 | 1.00 | 174.30 | A |
| ATOM | 2762 | OD2 | ASP | A | 347 | −50.184 | −26.499 | 5.840 | 1.00 | 174.30 | A |
| ATOM | 2763 | C | ASP | A | 347 | −49.629 | −25.659 | 2.561 | 1.00 | 192.07 | A |
| ATOM | 2764 | O | ASP | A | 347 | −50.332 | −24.668 | 2.351 | 1.00 | 192.07 | A |
| ATOM | 2765 | N | GLY | A | 348 | −48.349 | −25.577 | 2.915 | 1.00 | 190.33 | A |
| ATOM | 2766 | CA | GLY | A | 348 | −47.700 | −24.287 | 3.073 | 1.00 | 187.64 | A |
| ATOM | 2767 | C | GLY | A | 348 | −46.908 | −23.911 | 1.836 | 1.00 | 184.60 | A |
| ATOM | 2768 | O | GLY | A | 348 | −46.383 | −24.781 | 1.141 | 1.00 | 179.61 | A |
| ATOM | 2769 | N | ASN | A | 349 | −46.812 | −22.615 | 1.558 | 1.00 | 200.72 | A |
| ATOM | 2770 | CA | ASN | A | 349 | −46.085 | −22.152 | 0.385 | 1.00 | 200.62 | A |
| ATOM | 2771 | CB | ASN | A | 349 | −45.987 | −20.620 | 0.393 | 1.00 | 119.15 | A |
| ATOM | 2772 | CG | ASN | A | 349 | −45.821 | −20.047 | 1.786 | 1.00 | 104.33 | A |
| ATOM | 2773 | OD1 | ASN | A | 349 | −46.693 | −20.191 | 2.645 | 1.00 | 104.33 | A |
| ATOM | 2774 | ND2 | ASN | A | 349 | −44.698 | −19.387 | 2.016 | 1.00 | 104.33 | A |
| ATOM | 2775 | C | ASN | A | 349 | −44.700 | −22.783 | 0.210 | 1.00 | 200.72 | A |
| ATOM | 2776 | O | ASN | A | 349 | −44.565 | −23.770 | −0.516 | 1.00 | 200.72 | A |
| ATOM | 2777 | N | MET | A | 350 | −43.681 | −22.239 | 0.874 | 1.00 | 90.70 | A |
| ATOM | 2778 | CA | MET | A | 350 | −42.324 | −22.771 | 0.735 | 1.00 | 86.79 | A |
| ATOM | 2779 | CB | MET | A | 350 | −41.432 | −21.713 | 0.083 | 1.00 | 165.92 | A |
| ATOM | 2780 | CG | MET | A | 350 | −39.993 | −22.142 | −0.109 | 1.00 | 120.60 | A |
| ATOM | 2781 | SD | MET | A | 350 | −39.026 | −20.915 | −0.987 | 1.00 | 120.60 | A |
| ATOM | 2782 | CE | MET | A | 350 | −38.974 | −21.657 | −2.652 | 1.00 | 120.60 | A |
| ATOM | 2783 | C | MET | A | 350 | −41.667 | −23.271 | 2.030 | 1.00 | 83.93 | A |
| ATOM | 2784 | O | MET | A | 350 | −42.023 | −22.848 | 3.131 | 1.00 | 80.36 | A |
| ATOM | 2785 | N | THR | A | 351 | −40.702 | −24.181 | 1.878 | 1.00 | 98.62 | A |
| ATOM | 2786 | CA | THR | A | 351 | −39.972 | −24.749 | 3.011 | 1.00 | 96.58 | A |
| ATOM | 2787 | CB | THR | A | 351 | −40.475 | −26.185 | 3.348 | 1.00 | 100.87 | A |
| ATOM | 2788 | OG1 | THR | A | 351 | −40.413 | −26.385 | 4.760 | 1.00 | 67.99 | A |
| ATOM | 2789 | CG2 | THR | A | 351 | −39.611 | −27.261 | 2.669 | 1.00 | 67.99 | A |
| ATOM | 2790 | C | THR | A | 351 | −38.470 | −24.805 | 2.710 | 1.00 | 90.81 | A |
| ATOM | 2791 | O | THR | A | 351 | −38.052 | −25.220 | 1.626 | 1.00 | 82.40 | A |
| ATOM | 2792 | N | LEU | A | 352 | −37.657 | −24.379 | 3.670 | 1.00 | 61.55 | A |
| ATOM | 2793 | CA | LEU | A | 352 | −36.214 | −24.400 | 3.489 | 1.00 | 64.57 | A |
| ATOM | 2794 | CB | LEU | A | 352 | −35.625 | −23.021 | 3.768 | 1.00 | 81.16 | A |
| ATOM | 2795 | CG | LEU | A | 352 | −35.819 | −22.080 | 2.582 | 1.00 | 62.22 | A |
| ATOM | 2796 | CD1 | LEU | A | 352 | −35.500 | −20.660 | 2.972 | 1.00 | 62.22 | A |
| ATOM | 2797 | CD2 | LEU | A | 352 | −34.927 | −22.545 | 1.444 | 1.00 | 62.22 | A |
| ATOM | 2798 | C | LEU | A | 352 | −35.580 | −25.444 | 4.387 | 1.00 | 61.25 | A |
| ATOM | 2799 | O | LEU | A | 352 | −35.941 | −25.580 | 5.556 | 1.00 | 56.87 | A |
| ATOM | 2800 | N | GLU | A | 353 | −34.640 | −26.192 | 3.827 | 1.00 | 67.27 | A |
| ATOM | 2801 | CA | GLU | A | 353 | −33.971 | −27.239 | 4.566 | 1.00 | 63.52 | A |
| ATOM | 2802 | CB | GLU | A | 353 | −34.405 | −28.596 | 4.035 | 1.00 | 164.60 | A |
| ATOM | 2803 | CG | GLU | A | 353 | −33.778 | −29.754 | 4.758 | 1.00 | 123.15 | A |
| ATOM | 2804 | CD | GLU | A | 353 | −34.169 | −31.073 | 4.146 | 1.00 | 123.15 | A |
| ATOM | 2805 | OE1 | GLU | A | 353 | −35.385 | −31.339 | 4.052 | 1.00 | 123.15 | A |
| ATOM | 2806 | OE2 | GLU | A | 353 | −33.264 | −31.843 | 3.756 | 1.00 | 123.15 | A |
| ATOM | 2807 | C | GLU | A | 353 | −32.466 | −27.114 | 4.450 | 1.00 | 66.77 | A |
| ATOM | 2808 | O | GLU | A | 353 | −31.899 | −27.265 | 3.363 | 1.00 | 67.55 | A |
| ATOM | 2809 | N | CYS | A | 354 | −31.825 | −26.848 | 5.585 | 1.00 | 64.81 | A |
| ATOM | 2810 | CA | CYS | A | 354 | −30.375 | −26.708 | 5.659 | 1.00 | 65.09 | A |
| ATOM | 2811 | C | CYS | A | 354 | −29.843 | −28.043 | 6.145 | 1.00 | 66.18 | A |
| ATOM | 2812 | O | CYS | A | 354 | −30.235 | −28.501 | 7.207 | 1.00 | 64.88 | A |
| ATOM | 2813 | CB | CYS | A | 354 | −30.019 | −25.616 | 6.666 | 1.00 | 107.51 | A |
| ATOM | 2814 | SG | CYS | A | 354 | −28.312 | −24.997 | 6.614 | 1.00 | 84.70 | A |
| ATOM | 2815 | N | THR | A | 355 | −28.979 | −28.689 | 5.370 | 1.00 | 75.13 | A |
| ATOM | 2816 | CA | THR | A | 355 | −28.433 | −29.962 | 5.816 | 1.00 | 64.49 | A |
| ATOM | 2817 | CB | THR | A | 355 | −28.833 | −31.125 | 4.903 | 1.00 | 116.37 | A |
| ATOM | 2818 | OG1 | THR | A | 355 | −27.751 | −31.418 | 4.016 | 1.00 | 65.52 | A |
| ATOM | 2819 | CG2 | THR | A | 355 | −30.068 | −30.777 | 4.099 | 1.00 | 65.52 | A |
| ATOM | 2820 | C | THR | A | 355 | −26.917 | −29.893 | 5.870 | 1.00 | 77.98 | A |
| ATOM | 2821 | O | THR | A | 355 | −26.249 | −29.529 | 4.892 | 1.00 | 70.78 | A |
| ATOM | 2822 | N | VAL | A | 356 | −26.389 | −30.246 | 7.034 | 1.00 | 44.17 | A |
| ATOM | 2823 | CA | VAL | A | 356 | −24.956 | −30.236 | 7.283 | 1.00 | 39.89 | A |
| ATOM | 2824 | CB | VAL | A | 356 | −24.656 | −29.473 | 8.599 | 1.00 | 50.96 | A |
| ATOM | 2825 | CG1 | VAL | A | 356 | −23.160 | −29.221 | 8.735 | 1.00 | 35.49 | A |
| ATOM | 2826 | CG2 | VAL | A | 356 | −25.429 | −28.163 | 8.613 | 1.00 | 35.49 | A |
| ATOM | 2827 | C | VAL | A | 356 | −24.492 | −31.690 | 7.367 | 1.00 | 48.73 | A |
| ATOM | 2828 | O | VAL | A | 356 | −24.952 | −32.444 | 8.210 | 1.00 | 49.84 | A |
| ATOM | 2829 | N | ASN | A | 357 | −23.571 | −32.069 | 6.488 | 1.00 | 55.95 | A |
| ATOM | 2830 | CA | ASN | A | 357 | −23.102 | −33.443 | 6.426 | 1.00 | 52.17 | A |
| ATOM | 2831 | CB | ASN | A | 357 | −23.134 | −33.924 | 4.984 | 1.00 | 135.75 | A |
| ATOM | 2832 | CG | ASN | A | 357 | −24.526 | −33.958 | 4.434 | 1.00 | 89.67 | A |
| ATOM | 2833 | OD1 | ASN | A | 357 | −25.415 | −34.565 | 5.030 | 1.00 | 89.67 | A |
| ATOM | 2834 | ND2 | ASN | A | 357 | −24.736 | −33.298 | 3.297 | 1.00 | 89.67 | A |
| ATOM | 2835 | C | ASN | A | 357 | −21.752 | −33.772 | 7.009 | 1.00 | 56.33 | A |
| ATOM | 2836 | O | ASN | A | 357 | −20.726 | −33.162 | 6.674 | 1.00 | 59.21 | A |
| ATOM | 2837 | N | SER | A | 358 | −21.791 | −34.791 | 7.864 | 1.00 | 38.36 | A |
| ATOM | 2838 | CA | SER | A | 358 | −20.647 | −35.340 | 8.576 | 1.00 | 45.66 | A |
| ATOM | 2839 | CB | SER | A | 358 | −20.020 | −36.475 | 7.758 | 1.00 | 63.65 | A |
| ATOM | 2840 | OG | SER | A | 358 | −19.433 | −35.985 | 6.576 | 1.00 | 61.99 | A |
| ATOM | 2841 | C | SER | A | 358 | −19.579 | −34.337 | 9.020 | 1.00 | 50.38 | A |
| ATOM | 2842 | O | SER | A | 358 | −18.615 | −34.053 | 8.304 | 1.00 | 45.93 | A |
| ATOM | 2843 | N | PHE | A | 359 | −19.783 | −33.822 | 10.234 | 1.00 | 34.62 | A |
| ATOM | 2844 | CA | PHE | A | 359 | −18.890 | −32.859 | 10.901 | 1.00 | 28.93 | A |
| ATOM | 2845 | CB | PHE | A | 359 | −19.654 | −31.568 | 11.161 | 1.00 | 51.07 | A |
| ATOM | 2846 | CG | PHE | A | 359 | −21.009 | −31.785 | 11.754 | 1.00 | 45.67 | A |
| ATOM | 2847 | CD1 | PHE | A | 359 | −21.163 | −31.951 | 13.128 | 1.00 | 45.67 | A |
| ATOM | 2848 | CD2 | PHE | A | 359 | −22.144 | −31.781 | 10.943 | 1.00 | 45.67 | A |
| ATOM | 2849 | CE1 | PHE | A | 359 | −22.436 | −32.103 | 13.696 | 1.00 | 45.67 | A |
| ATOM | 2850 | CE2 | PHE | A | 359 | −23.425 | −31.931 | 11.492 | 1.00 | 45.67 | A |
| ATOM | 2851 | CZ | PHE | A | 359 | −23.572 | −32.090 | 12.873 | 1.00 | 45.67 | A |
| ATOM | 2852 | C | PHE | A | 359 | −18.455 | −33.493 | 12.227 | 1.00 | 35.52 | A |
| ATOM | 2853 | O | PHE | A | 359 | −19.260 | −34.203 | 12.835 | 1.00 | 33.07 | A |
| ATOM | 2854 | N | TYR | A | 360 | −17.219 | −33.274 | 12.692 | 1.00 | 90.31 | A |
| ATOM | 2855 | CA | TYR | A | 360 | −16.905 | −33.946 | 13.931 | 1.00 | 90.09 | A |
| ATOM | 2856 | CB | TYR | A | 360 | −15.431 | −34.250 | 14.180 | 1.00 | 157.30 | A |
| ATOM | 2857 | CG | TYR | A | 360 | −15.324 | −34.757 | 15.620 | 1.00 | 44.44 | A |
| ATOM | 2858 | CD1 | TYR | A | 360 | −16.237 | −35.721 | 16.108 | 1.00 | 44.44 | A |
| ATOM | 2859 | CE1 | TYR | A | 360 | −16.286 | −36.071 | 17.470 | 1.00 | 44.44 | A |
| ATOM | 2860 | CD2 | TYR | A | 360 | −14.444 | −34.170 | 16.540 | 1.00 | 44.44 | A |
| ATOM | 2861 | CE2 | TYR | A | 360 | −14.485 | −34.523 | 17.913 | 1.00 | 44.44 | A |
| ATOM | 2862 | CZ | TYR | A | 360 | −15.411 | −35.470 | 18.363 | 1.00 | 44.44 | A |
| ATOM | 2863 | OH | TYR | A | 360 | −15.454 | −35.811 | 19.698 | 1.00 | 44.44 | A |
| ATOM | 2864 | C | TYR | A | 360 | −17.417 | −33.368 | 15.210 | 1.00 | 91.18 | A |
| ATOM | 2865 | O | TYR | A | 360 | −18.431 | −33.836 | 15.697 | 1.00 | 97.11 | A |
| ATOM | 2866 | N | PRO | A | 361 | −16.721 | −32.380 | 15.802 | 1.00 | 61.94 | A |
| ATOM | 2867 | CD | PRO | A | 361 | −15.850 | −31.365 | 15.189 | 1.00 | 31.16 | A |
| ATOM | 2868 | CA | PRO | A | 361 | −17.322 | −31.913 | 17.067 | 1.00 | 57.53 | A |
| ATOM | 2869 | CB | PRO | A | 361 | −16.603 | −30.603 | 17.342 | 1.00 | 30.40 | A |
| ATOM | 2870 | CG | PRO | A | 361 | −16.230 | −30.116 | 15.972 | 1.00 | 31.16 | A |
| ATOM | 2871 | C | PRO | A | 361 | −18.821 | −31.817 | 16.793 | 1.00 | 64.93 | A |
| ATOM | 2872 | O | PRO | A | 361 | −19.267 | −31.039 | 15.958 | 1.00 | 62.10 | A |
| ATOM | 2873 | N | PRO | A | 362 | −19.610 | −32.683 | 17.453 | 1.00 | 45.90 | A |
| ATOM | 2874 | CD | PRO | A | 362 | −19.100 | −33.574 | 18.513 | 1.00 | 35.37 | A |
| ATOM | 2875 | CA | PRO | A | 362 | −21.070 | −32.794 | 17.324 | 1.00 | 39.61 | A |
| ATOM | 2876 | CB | PRO | A | 362 | −21.416 | −33.969 | 18.239 | 1.00 | 35.37 | A |
| ATOM | 2877 | CG | PRO | A | 362 | −20.346 | −33.917 | 19.272 | 1.00 | 35.37 | A |
| ATOM | 2878 | C | PRO | A | 362 | −21.936 | −31.583 | 17.567 | 1.00 | 42.21 | A |
| ATOM | 2879 | O | PRO | A | 362 | −23.109 | −31.577 | 17.185 | 1.00 | 46.97 | A |
| ATOM | 2880 | N | ASP | A | 363 | −21.372 | −30.560 | 18.199 | 1.00 | 40.85 | A |
| ATOM | 2881 | CA | ASP | A | 363 | −22.116 | −29.336 | 18.453 | 1.00 | 40.70 | A |
| ATOM | 2882 | CB | ASP | A | 363 | −21.479 | −28.533 | 19.604 | 1.00 | 70.34 | A |
| ATOM | 2883 | CG | ASP | A | 363 | −19.959 | −28.380 | 19.470 | 1.00 | 64.55 | A |
| ATOM | 2884 | OD1 | ASP | A | 363 | −19.248 | −29.410 | 19.399 | 1.00 | 64.55 | A |
| ATOM | 2885 | OD2 | ASP | A | 363 | −19.473 | −27.225 | 19.458 | 1.00 | 64.55 | A |
| ATOM | 2886 | C | ASP | A | 363 | −22.188 | −28.484 | 17.191 | 1.00 | 44.24 | A |
| ATOM | 2887 | O | ASP | A | 363 | −21.167 | −28.128 | 16.592 | 1.00 | 40.74 | A |
| ATOM | 2888 | N | VAL | A | 364 | −23.407 | −28.174 | 16.775 | 1.00 | 41.77 | A |
| ATOM | 2889 | CA | VAL | A | 364 | −23.619 | −27.351 | 15.597 | 1.00 | 44.09 | A |
| ATOM | 2890 | CB | VAL | A | 364 | −23.912 | −28.189 | 14.335 | 1.00 | 61.02 | A |
| ATOM | 2891 | CG1 | VAL | A | 364 | −24.592 | −27.338 | 13.290 | 1.00 | 39.41 | A |
| ATOM | 2892 | CG2 | VAL | A | 364 | −22.623 | −28.706 | 13.763 | 1.00 | 39.41 | A |
| ATOM | 2893 | C | VAL | A | 364 | −24.814 | −26.490 | 15.879 | 1.00 | 51.43 | A |
| ATOM | 2894 | O | VAL | A | 364 | −25.766 | −26.925 | 16.519 | 1.00 | 43.17 | A |
| ATOM | 2895 | N | ILE | A | 365 | −24.750 | −25.251 | 15.426 | 1.00 | 53.02 | A |
| ATOM | 2896 | CA | ILE | A | 365 | −25.863 | −24.354 | 15.611 | 1.00 | 47.07 | A |
| ATOM | 2897 | CB | ILE | A | 365 | −25.601 | −23.312 | 16.700 | 1.00 | 73.93 | A |
| ATOM | 2898 | CG2 | ILE | A | 365 | −26.725 | −22.297 | 16.717 | 1.00 | 50.37 | A |
| ATOM | 2899 | CG1 | ILE | A | 365 | −25.527 | −24.014 | 18.059 | 1.00 | 50.37 | A |
| ATOM | 2900 | CD1 | ILE | A | 365 | −25.264 | −23.099 | 19.222 | 1.00 | 50.37 | A |
| ATOM | 2901 | C | ILE | A | 365 | −26.145 | −23.687 | 14.296 | 1.00 | 53.83 | A |
| ATOM | 2902 | O | ILE | A | 365 | −25.242 | −23.391 | 13.514 | 1.00 | 54.60 | A |
| ATOM | 2903 | N | THR | A | 366 | −27.423 | −23.451 | 14.070 | 1.00 | 36.52 | A |
| ATOM | 2904 | CA | THR | A | 366 | −27.890 | −22.880 | 12.838 | 1.00 | 44.80 | A |
| ATOM | 2905 | CB | THR | A | 366 | −28.689 | −23.953 | 12.087 | 1.00 | 57.45 | A |
| ATOM | 2906 | OG1 | THR | A | 366 | −28.095 | −24.168 | 10.816 | 1.00 | 45.13 | A |
| ATOM | 2907 | CG2 | THR | A | 366 | −30.131 | −23.555 | 11.915 | 1.00 | 45.13 | A |
| ATOM | 2908 | C | THR | A | 366 | −28.747 | −21.646 | 13.059 | 1.00 | 53.78 | A |
| ATOM | 2909 | O | THR | A | 366 | −29.617 | −21.623 | 13.942 | 1.00 | 53.02 | A |
| ATOM | 2910 | N | LYS | A | 367 | −28.487 | −20.623 | 12.247 | 1.00 | 46.12 | A |
| ATOM | 2911 | CA | LYS | A | 367 | −29.249 | −19.373 | 12.295 | 1.00 | 49.32 | A |
| ATOM | 2912 | CB | LYS | A | 367 | −28.372 | −18.198 | 12.754 | 1.00 | 57.37 | A |
| ATOM | 2913 | CG | LYS | A | 367 | −28.012 | −18.227 | 14.210 | 1.00 | 48.38 | A |
| ATOM | 2914 | CD | LYS | A | 367 | −27.383 | −16.938 | 14.673 | 1.00 | 48.38 | A |
| ATOM | 2915 | CE | LYS | A | 367 | −27.231 | −16.964 | 16.188 | 1.00 | 48.38 | A |
| ATOM | 2916 | NZ | LYS | A | 367 | −26.923 | −15.621 | 16.766 | 1.00 | 48.38 | A |
| ATOM | 2917 | C | LYS | A | 367 | −29.828 | −19.045 | 10.925 | 1.00 | 52.02 | A |
| ATOM | 2918 | O | LYS | A | 367 | −29.123 | −19.072 | 9.917 | 1.00 | 52.33 | A |
| ATOM | 2919 | N | TRP | A | 368 | −31.121 | −18.751 | 10.902 | 1.00 | 49.37 | A |
| ATOM | 2920 | CA | TRP | A | 368 | −31.795 | −18.371 | 9.665 | 1.00 | 49.43 | A |
| ATOM | 2921 | CB | TRP | A | 368 | −33.150 | −19.074 | 9.534 | 1.00 | 78.05 | A |
| ATOM | 2922 | CG | TRP | A | 368 | −33.012 | −20.530 | 9.290 | 1.00 | 57.97 | A |
| ATOM | 2923 | CD2 | TRP | A | 368 | −32.851 | −21.176 | 8.020 | 1.00 | 57.97 | A |
| ATOM | 2924 | CE2 | TRP | A | 368 | −32.702 | −22.556 | 8.270 | 1.00 | 57.97 | A |
| ATOM | 2925 | CE3 | TRP | A | 368 | −32.816 | −20.723 | 6.696 | 1.00 | 57.97 | A |
| ATOM | 2926 | CD1 | TRP | A | 368 | −32.956 | −21.510 | 10.229 | 1.00 | 57.97 | A |
| ATOM | 2927 | NE1 | TRP | A | 368 | −32.771 | −22.732 | 9.627 | 1.00 | 57.97 | A |
| ATOM | 2928 | CZ2 | TRP | A | 368 | −32.523 | −23.492 | 7.241 | 1.00 | 57.97 | A |
| ATOM | 2929 | CZ3 | TRP | A | 368 | −32.637 | −21.654 | 5.674 | 1.00 | 57.97 | A |
| ATOM | 2930 | CH2 | TRP | A | 368 | −32.493 | −23.023 | 5.955 | 1.00 | 57.97 | A |
| ATOM | 2931 | C | TRP | A | 368 | −31.984 | −16.858 | 9.654 | 1.00 | 51.38 | A |
| ATOM | 2932 | O | TRP | A | 368 | −32.695 | −16.302 | 10.495 | 1.00 | 51.57 | A |
| ATOM | 2933 | N | ILE | A | 369 | −31.337 | −16.203 | 8.694 | 1.00 | 58.14 | A |
| ATOM | 2934 | CA | ILE | A | 369 | −31.403 | −14.752 | 8.577 | 1.00 | 59.51 | A |
| ATOM | 2935 | CB | ILE | A | 369 | −30.031 | −14.142 | 8.879 | 1.00 | 58.11 | A |
| ATOM | 2936 | CG2 | ILE | A | 369 | −30.113 | −12.649 | 8.829 | 1.00 | 35.79 | A |
| ATOM | 2937 | CG1 | ILE | A | 369 | −29.551 | −14.606 | 10.257 | 1.00 | 35.79 | A |
| ATOM | 2938 | CD1 | ILE | A | 369 | −28.145 | −14.203 | 10.577 | 1.00 | 35.79 | A |
| ATOM | 2939 | C | ILE | A | 369 | −31.840 | −14.304 | 7.187 | 1.00 | 61.33 | A |
| ATOM | 2940 | O | ILE | A | 369 | −31.345 | −14.815 | 6.181 | 1.00 | 58.13 | A |
| ATOM | 2941 | N | GLU | A | 370 | −32.768 | −13.354 | 7.127 | 1.00 | 57.39 | A |
| ATOM | 2942 | CA | GLU | A | 370 | −33.234 | −12.856 | 5.841 | 1.00 | 60.04 | A |
| ATOM | 2943 | CB | GLU | A | 370 | −34.318 | −11.805 | 6.056 | 1.00 | 119.15 | A |
| ATOM | 2944 | CG | GLU | A | 370 | −35.654 | −12.433 | 6.409 | 1.00 | 97.79 | A |
| ATOM | 2945 | CD | GLU | A | 370 | −36.680 | −11.427 | 6.882 | 1.00 | 97.79 | A |
| ATOM | 2946 | OE1 | GLU | A | 370 | −36.853 | −10.386 | 6.211 | 1.00 | 97.79 | A |
| ATOM | 2947 | OE2 | GLU | A | 370 | −37.325 | −11.682 | 7.923 | 1.00 | 97.79 | A |
| ATOM | 2948 | C | GLU | A | 370 | −32.032 | −12.288 | 5.090 | 1.00 | 60.22 | A |
| ATOM | 2949 | O | GLU | A | 370 | −31.247 | −11.528 | 5.657 | 1.00 | 59.07 | A |
| ATOM | 2950 | N | SER | A | 371 | −31.884 | −12.669 | 3.822 | 1.00 | 67.72 | A |
| ATOM | 2951 | CA | SER | A | 371 | −30.749 | −12.228 | 3.005 | 1.00 | 68.26 | A |
| ATOM | 2952 | CB | SER | A | 371 | −30.831 | −12.857 | 1.616 | 1.00 | 73.06 | A |
| ATOM | 2953 | OG | SER | A | 371 | −32.040 | −12.499 | 0.982 | 1.00 | 62.14 | A |
| ATOM | 2954 | C | SER | A | 371 | −30.547 | −10.718 | 2.862 | 1.00 | 67.53 | A |
| ATOM | 2955 | O | SER | A | 371 | −29.433 | −10.264 | 2.581 | 1.00 | 68.47 | A |
| ATOM | 2956 | N | GLU | A | 372 | −31.612 | −9.942 | 3.040 | 1.00 | 57.96 | A |
| ATOM | 2957 | CA | GLU | A | 372 | −31.503 | −8.490 | 2.930 | 1.00 | 56.51 | A |
| ATOM | 2958 | CB | GLU | A | 372 | −32.838 | −7.824 | 3.264 | 1.00 | 105.79 | A |
| ATOM | 2959 | CG | GLU | A | 372 | −33.351 | −8.161 | 4.647 | 1.00 | 105.79 | A |
| ATOM | 2960 | CD | GLU | A | 372 | −34.483 | −7.256 | 5.091 | 1.00 | 105.79 | A |
| ATOM | 2961 | OE1 | GLU | A | 372 | −34.240 | −6.038 | 5.227 | 1.00 | 105.79 | A |
| ATOM | 2962 | OE2 | GLU | A | 372 | −35.611 | −7.760 | 5.308 | 1.00 | 105.79 | A |
| ATOM | 2963 | C | GLU | A | 372 | −30.426 | −7.969 | 3.883 | 1.00 | 61.12 | A |
| ATOM | 2964 | O | GLU | A | 372 | −29.654 | −7.071 | 3.545 | 1.00 | 62.19 | A |
| ATOM | 2965 | N | HIS | A | 373 | −30.373 | −8.554 | 5.072 | 1.00 | 60.93 | A |
| ATOM | 2966 | CA | HIS | A | 373 | −29.417 | −8.149 | 6.090 | 1.00 | 64.42 | A |
| ATOM | 2967 | CB | HIS | A | 373 | −29.733 | −8.862 | 7.392 | 1.00 | 97.69 | A |
| ATOM | 2968 | CG | HIS | A | 373 | −31.083 | −8.531 | 7.928 | 1.00 | 67.04 | A |
| ATOM | 2969 | CD2 | HIS | A | 373 | −32.124 | −9.322 | 8.278 | 1.00 | 67.04 | A |
| ATOM | 2970 | ND1 | HIS | A | 373 | −31.486 | −7.235 | 8.163 | 1.00 | 67.04 | A |
| ATOM | 2971 | CE1 | HIS | A | 373 | −32.719 | −7.241 | 8.638 | 1.00 | 67.04 | A |
| ATOM | 2972 | NE2 | HIS | A | 373 | −33.130 | −8.495 | 8.717 | 1.00 | 67.04 | A |
| ATOM | 2973 | C | HIS | A | 373 | −27.950 | −8.356 | 5.744 | 1.00 | 60.19 | A |
| ATOM | 2974 | O | HIS | A | 373 | −27.072 | −7.904 | 6.476 | 1.00 | 56.03 | A |
| ATOM | 2975 | N | PHE | A | 374 | −27.669 | −9.041 | 4.647 | 1.00 | 52.21 | A |
| ATOM | 2976 | CA | PHE | A | 374 | −26.279 | −9.240 | 4.278 | 1.00 | 52.98 | A |
| ATOM | 2977 | CB | PHE | A | 374 | −26.054 | −10.665 | 3.780 | 1.00 | 76.24 | A |
| ATOM | 2978 | CG | PHE | A | 374 | −26.087 | −11.680 | 4.873 | 1.00 | 56.42 | A |
| ATOM | 2979 | CD1 | PHE | A | 374 | −27.296 | −12.173 | 5.350 | 1.00 | 56.42 | A |
| ATOM | 2980 | CD2 | PHE | A | 374 | −24.909 | −12.090 | 5.486 | 1.00 | 56.42 | A |
| ATOM | 2981 | CE1 | PHE | A | 374 | −27.334 | −13.055 | 6.424 | 1.00 | 56.42 | A |
| ATOM | 2982 | CE2 | PHE | A | 374 | −24.935 | −12.965 | 6.557 | 1.00 | 56.42 | A |
| ATOM | 2983 | CZ | PHE | A | 374 | −26.154 | −13.449 | 7.027 | 1.00 | 56.42 | A |
| ATOM | 2984 | C | PHE | A | 374 | −25.827 | −8.228 | 3.235 | 1.00 | 49.06 | A |
| ATOM | 2985 | O | PHE | A | 374 | −24.734 | −8.345 | 2.676 | 1.00 | 47.30 | A |
| ATOM | 2986 | N | LYS | A | 375 | −26.671 | −7.226 | 2.998 | 1.00 | 71.42 | A |
| ATOM | 2987 | CA | LYS | A | 375 | −26.391 | −6.175 | 2.027 | 1.00 | 74.05 | A |
| ATOM | 2988 | CB | LYS | A | 375 | −25.816 | −4.945 | 2.734 | 1.00 | 121.77 | A |
| ATOM | 2989 | CG | LYS | A | 375 | −26.842 | −4.047 | 3.412 | 1.00 | 95.61 | A |
| ATOM | 2990 | CD | LYS | A | 375 | −27.460 | −4.687 | 4.643 | 1.00 | 95.61 | A |
| ATOM | 2991 | CE | LYS | A | 375 | −28.411 | −3.715 | 5.355 | 1.00 | 95.61 | A |
| ATOM | 2992 | NZ | LYS | A | 375 | −29.075 | −4.316 | 6.554 | 1.00 | 95.61 | A |
| ATOM | 2993 | C | LYS | A | 375 | −25.450 | −6.591 | 0.892 | 1.00 | 79.07 | A |
| ATOM | 2994 | O | LYS | A | 375 | −24.417 | −5.961 | 0.679 | 1.00 | 81.33 | A |
| ATOM | 2995 | N | GLY | A | 376 | −25.794 | −7.657 | 0.174 | 1.00 | 67.39 | A |
| ATOM | 2996 | CA | GLY | A | 376 | −24.962 | −8.088 | −0.937 | 1.00 | 67.39 | A |
| ATOM | 2997 | C | GLY | A | 376 | −23.766 | −8.979 | −0.653 | 1.00 | 67.39 | A |
| ATOM | 2998 | O | GLY | A | 376 | −23.393 | −9.780 | −1.507 | 1.00 | 67.39 | A |
| ATOM | 2999 | N | GLU | A | 377 | −23.147 | −8.843 | 0.517 | 1.00 | 79.80 | A |
| ATOM | 3000 | CA | GLU | A | 377 | −21.994 | −9.671 | 0.859 | 1.00 | 79.80 | A |
| ATOM | 3001 | CB | GLU | A | 377 | −20.909 | −8.827 | 1.523 | 1.00 | 102.70 | A |
| ATOM | 3002 | CG | GLU | A | 377 | −19.584 | −9.546 | 1.673 | 1.00 | 102.70 | A |
| ATOM | 3003 | CD | GLU | A | 377 | −18.513 | −8.668 | 2.290 | 1.00 | 102.70 | A |
| ATOM | 3004 | OE1 | GLU | A | 377 | −17.343 | −9.109 | 2.322 | 1.00 | 102.70 | A |
| ATOM | 3005 | OE2 | GLU | A | 377 | −18.842 | −7.544 | 2.743 | 1.00 | 102.70 | A |
| ATOM | 3006 | C | GLU | A | 377 | −22.437 | −10.771 | 1.809 | 1.00 | 79.80 | A |
| ATOM | 3007 | O | GLU | A | 377 | −22.900 | −10.491 | 2.922 | 1.00 | 79.80 | A |
| ATOM | 3008 | N | TYR | A | 376 | −22.300 | −12.022 | 1.373 | 1.00 | 55.39 | A |
| ATOM | 3009 | CA | TYR | A | 378 | −22.703 | −13.162 | 2.204 | 1.00 | 55.39 | A |
| ATOM | 3010 | CB | TYR | A | 378 | −23.253 | −14.275 | 1.319 | 1.00 | 77.89 | A |
| ATOM | 3011 | CG | TYR | A | 378 | −24.570 | −13.897 | 0.701 | 1.00 | 77.89 | A |
| ATOM | 3012 | CD1 | TYR | A | 378 | −24.631 | −13.320 | −0.566 | 1.00 | 77.89 | A |
| ATOM | 3013 | CE1 | TYR | A | 378 | −25.847 | −12.889 | −1.102 | 1.00 | 77.89 | A |
| ATOM | 3014 | CD2 | TYR | A | 378 | −25.754 | −14.040 | 1.416 | 1.00 | 77.89 | A |
| ATOM | 3015 | CE2 | TYR | A | 378 | −26.968 | −13.614 | 0.893 | 1.00 | 77.89 | A |
| ATOM | 3016 | CZ | TYR | A | 378 | −27.010 | −13.037 | −0.362 | 1.00 | 77.89 | A |
| ATOM | 3017 | OH | TYR | A | 378 | −28.206 | −12.577 | −0.859 | 1.00 | 77.89 | A |
| ATOM | 3018 | C | TYR | A | 378 | −21.647 | −13.730 | 3.159 | 1.00 | 55.39 | A |
| ATOM | 3019 | O | TYR | A | 378 | −21.184 | −14.862 | 3.002 | 1.00 | 55.39 | A |
| ATOM | 3020 | N | LYS | A | 379 | −21.276 | −12.910 | 4.140 | 1.00 | 51.86 | A |
| ATOM | 3021 | CA | LYS | A | 379 | −20.315 | −13.253 | 5.179 | 1.00 | 51.86 | A |
| ATOM | 3022 | CB | LYS | A | 379 | −18.986 | −12.518 | 4.982 | 1.00 | 79.55 | A |
| ATOM | 3023 | CG | LYS | A | 379 | −18.199 | −12.955 | 3.761 | 1.00 | 79.55 | A |
| ATOM | 3024 | CD | LYS | A | 379 | −16.913 | −12.143 | 3.631 | 1.00 | 79.55 | A |
| ATOM | 3025 | CE | LYS | A | 379 | −16.187 | −12.413 | 2.309 | 1.00 | 79.55 | A |
| ATOM | 3026 | NZ | LYS | A | 379 | −15.028 | −11.485 | 2.126 | 1.00 | 79.55 | A |
| ATOM | 3027 | C | LYS | A | 379 | −20.964 | −12.802 | 6.478 | 1.00 | 51.86 | A |
| ATOM | 3028 | O | LYS | A | 379 | −21.595 | −11.746 | 6.534 | 1.00 | 51.86 | A |
| ATOM | 3029 | N | TYR | A | 380 | −20.851 | −13.623 | 7.511 | 1.00 | 41.61 | A |
| ATOM | 3030 | CA | TYR | A | 380 | −21.412 | −13.279 | 8.812 | 1.00 | 41.61 | A |
| ATOM | 3031 | CB | TYR | A | 380 | −21.767 | −14.555 | 9.566 | 1.00 | 45.19 | A |
| ATOM | 3032 | CG | TYR | A | 380 | −22.458 | −14.322 | 10.875 | 1.00 | 45.19 | A |
| ATOM | 3033 | CD1 | TYR | A | 380 | −23.840 | −14.150 | 10.929 | 1.00 | 45.19 | A |
| ATOM | 3034 | CE1 | TYR | A | 380 | −24.493 | −13.930 | 12.154 | 1.00 | 45.19 | A |
| ATOM | 3035 | CD2 | TYR | A | 380 | −21.731 | −14.265 | 12.070 | 1.00 | 45.19 | A |
| ATOM | 3036 | CE2 | TYR | A | 380 | −22.365 | −14.042 | 13.294 | 1.00 | 45.19 | A |
| ATOM | 3037 | CZ | TYR | A | 380 | −23.746 | −13.878 | 13.332 | 1.00 | 45.19 | A |
| ATOM | 3038 | OH | TYR | A | 380 | −24.386 | −13.691 | 14.537 | 1.00 | 45.19 | A |
| ATOM | 3039 | C | TYR | A | 380 | −20.321 | −12.503 | 9.569 | 1.00 | 41.61 | A |
| ATOM | 3040 | O | TYR | A | 380 | −19.291 | −13.073 | 9.914 | 1.00 | 41.61 | A |
| ATOM | 3041 | N | VAL | A | 381 | −20.534 | −11.209 | 9.800 | 1.00 | 48.85 | A |
| ATOM | 3042 | CA | VAL | A | 381 | −19.547 | −10.391 | 10.502 | 1.00 | 48.85 | A |
| ATOM | 3043 | CB | VAL | A | 381 | −19.708 | −8.910 | 10.134 | 1.00 | 58.85 | A |
| ATOM | 3044 | CG1 | VAL | A | 381 | −18.612 | −8.090 | 10.784 | 1.00 | 58.85 | A |
| ATOM | 3045 | CG2 | VAL | A | 381 | −19.668 | −8.755 | 8.628 | 1.00 | 58.85 | A |
| ATOM | 3046 | C | VAL | A | 381 | −19.688 | −10.558 | 12.014 | 1.00 | 48.85 | A |
| ATOM | 3047 | O | VAL | A | 381 | −20.750 | −10.278 | 12.582 | 1.00 | 48.85 | A |
| ATOM | 3048 | N | ASN | A | 382 | −18.605 | −10.987 | 12.654 | 1.00 | 82.89 | A |
| ATOM | 3049 | CA | ASN | A | 382 | −18.609 | −11.247 | 14.082 | 1.00 | 82.89 | A |
| ATOM | 3050 | CB | ASN | A | 382 | −17.193 | −11.275 | 14.627 | 1.00 | 52.29 | A |
| ATOM | 3051 | CG | ASN | A | 382 | −17.074 | −12.132 | 15.892 | 1.00 | 52.29 | A |
| ATOM | 3052 | OD1 | ASN | A | 382 | −18.008 | −12.220 | 16.703 | 1.00 | 52.29 | A |
| ATOM | 3053 | ND2 | ASN | A | 382 | −15.918 | −12.754 | 16.069 | 1.00 | 52.29 | A |
| ATOM | 3054 | C | ASN | A | 382 | −19.442 | −10.320 | 14.955 | 1.00 | 82.89 | A |
| ATOM | 3055 | O | ASN | A | 382 | −20.564 | −10.666 | 15.361 | 1.00 | 82.89 | A |
| ATOM | 3056 | N | GLY | A | 383 | −18.913 | −9.144 | 15.256 | 1.00 | 40.80 | A |
| ATOM | 3057 | CA | GLY | A | 383 | −19.667 | −8.261 | 16.130 | 1.00 | 40.80 | A |
| ATOM | 3058 | C | GLY | A | 383 | −20.929 | −7.605 | 15.588 | 1.00 | 40.80 | A |
| ATOM | 3059 | O | GLY | A | 383 | −21.559 | −6.819 | 16.293 | 1.00 | 40.80 | A |
| ATOM | 3060 | N | ARG | A | 384 | −21.320 | −7.913 | 14.358 | 1.00 | 44.79 | A |
| ATOM | 3061 | CA | ARG | A | 384 | −22.498 | −7.279 | 13.780 | 1.00 | 44.79 | A |
| ATOM | 3062 | CB | ARG | A | 384 | −22.444 | −7.408 | 12.268 | 1.00 | 58.07 | A |
| ATOM | 3063 | CG | ARG | A | 384 | −23.501 | −6.609 | 11.541 | 1.00 | 58.07 | A |
| ATOM | 3064 | CD | ARG | A | 384 | −23.134 | −6.510 | 10.077 | 1.00 | 58.07 | A |
| ATOM | 3065 | NE | ARG | A | 384 | −21.847 | −5.850 | 9.920 | 1.00 | 58.07 | A |
| ATOM | 3066 | CZ | ARG | A | 384 | −21.187 | −5.751 | 8.770 | 1.00 | 58.07 | A |
| ATOM | 3067 | NH1 | ARG | A | 384 | −21.691 | −6.276 | 7.668 | 1.00 | 58.07 | A |
| ATOM | 3068 | NH2 | ARG | A | 384 | −20.021 | −5.117 | 8.714 | 1.00 | 58.07 | A |
| ATOM | 3069 | C | ARG | A | 384 | −23.818 | −7.807 | 14.285 | 1.00 | 44.79 | A |
| ATOM | 3070 | O | ARG | A | 384 | −23.924 | −8.984 | 14.619 | 1.00 | 44.79 | A |
| ATOM | 3071 | N | TYR | A | 385 | −24.823 | −6.939 | 14.347 | 1.00 | 56.08 | A |
| ATOM | 3072 | CA | TYR | A | 385 | −26.146 | −7.343 | 14.801 | 1.00 | 56.08 | A |
| ATOM | 3073 | CB | TYR | A | 385 | −26.897 | −6.147 | 15.380 | 1.00 | 63.99 | A |
| ATOM | 3074 | CG | TYR | A | 385 | −28.339 | −6.465 | 15.676 | 1.00 | 63.99 | A |
| ATOM | 3075 | CD1 | TYR | A | 385 | −28.692 | −7.230 | 16.788 | 1.00 | 63.99 | A |
| ATOM | 3076 | CE1 | TYR | A | 385 | −30.013 | −7.592 | 17.022 | 1.00 | 63.99 | A |
| ATOM | 3077 | CD2 | TYR | A | 385 | −29.346 | −6.066 | 14.806 | 1.00 | 63.99 | A |
| ATOM | 3078 | CE2 | TYR | A | 385 | −30.669 | −6.421 | 15.024 | 1.00 | 63.99 | A |
| ATOM | 3079 | CZ | TYR | A | 385 | −30.999 | −7.182 | 16.130 | 1.00 | 63.99 | A |
| ATOM | 3080 | OH | TYR | A | 385 | −32.318 | −7.532 | 16.337 | 1.00 | 63.99 | A |
| ATOM | 3081 | C | TYR | A | 385 | −26.966 | −7.930 | 13.644 | 1.00 | 56.08 | A |
| ATOM | 3082 | O | TYR | A | 385 | −26.867 | −7.448 | 12.516 | 1.00 | 56.08 | A |
| ATOM | 3083 | N | TYR | A | 386 | −27.770 | −8.961 | 13.919 | 1.00 | 67.96 | A |
| ATOM | 3084 | CA | TYR | A | 386 | −28.618 | −9.570 | 12.891 | 1.00 | 67.96 | A |
| ATOM | 3085 | CB | TYR | A | 386 | −27.959 | −10.791 | 12.241 | 1.00 | 44.87 | A |
| ATOM | 3086 | CG | TYR | A | 386 | −26.677 | −10.567 | 11.478 | 1.00 | 44.87 | A |
| ATOM | 3087 | CD1 | TYR | A | 386 | −25.435 | −10.776 | 12.084 | 1.00 | 44.87 | A |
| ATOM | 3088 | CE1 | TYR | A | 386 | −24.244 | −10.620 | 11.366 | 1.00 | 44.87 | A |
| ATOM | 3089 | CD2 | TYR | A | 386 | −26.699 | −10.190 | 10.138 | 1.00 | 44.87 | A |
| ATOM | 3090 | CE2 | TYR | A | 386 | −25.515 | −10.022 | 9.414 | 1.00 | 44.87 | A |
| ATOM | 3091 | CZ | TYR | A | 386 | −24.291 | −10.243 | 10.035 | 1.00 | 44.87 | A |
| ATOM | 3092 | OH | TYR | A | 386 | −23.120 | −10.087 | 9.335 | 1.00 | 44.87 | A |
| ATOM | 3093 | C | TYR | A | 386 | −29.939 | −10.079 | 13.444 | 1.00 | 67.96 | A |
| ATOM | 3094 | O | TYR | A | 386 | −29.951 | −10.876 | 14.378 | 1.00 | 67.96 | A |
| ATOM | 3095 | N | PRO | A | 387 | −31.072 | −9.626 | 12.887 | 1.00 | 60.30 | A |
| ATOM | 3096 | CD | PRO | A | 387 | −31.253 | −8.503 | 11.954 | 1.00 | 77.61 | A |
| ATOM | 3097 | CA | PRO | A | 387 | −32.367 | −10.122 | 13.379 | 1.00 | 60.30 | A |
| ATOM | 3098 | CB | PRO | A | 387 | −33.366 | −9.325 | 12.555 | 1.00 | 77.61 | A |
| ATOM | 3099 | CG | PRO | A | 387 | −32.636 | −8.032 | 12.305 | 1.00 | 77.61 | A |
| ATOM | 3100 | C | PRO | A | 387 | −32.309 | −11.597 | 12.950 | 1.00 | 60.30 | A |
| ATOM | 3101 | O | PRO | A | 387 | −31.731 | −11.885 | 11.892 | 1.00 | 60.30 | A |
| ATOM | 3102 | N | GLU | A | 388 | −32.884 | −12.538 | 13.696 | 1.00 | 90.02 | A |
| ATOM | 3103 | CA | GLU | A | 388 | −32.691 | −13.907 | 13.244 | 1.00 | 90.02 | A |
| ATOM | 3104 | CB | GLU | A | 388 | −31.507 | −14.505 | 14.002 | 1.00 | 145.71 | A |
| ATOM | 3105 | CG | GLU | A | 388 | −31.766 | −14.748 | 15.469 | 1.00 | 145.71 | A |
| ATOM | 3106 | CD | GLU | A | 388 | −30.683 | −15.590 | 16.103 | 1.00 | 145.71 | A |
| ATOM | 3107 | OE1 | GLU | A | 388 | −30.463 | −16.723 | 15.624 | 1.00 | 145.71 | A |
| ATOM | 3108 | OE2 | GLU | A | 388 | −30.053 | −15.123 | 17.076 | 1.00 | 145.71 | A |
| ATOM | 3109 | C | GLU | A | 388 | −33.755 | −14.993 | 13.140 | 1.00 | 90.02 | A |
| ATOM | 3110 | O | GLU | A | 388 | −33.392 | −16.164 | 13.088 | 1.00 | 90.02 | A |
| ATOM | 3111 | N | TRP | A | 389 | −35.040 | −14.672 | 13.092 | 1.00 | 86.89 | A |
| ATOM | 3112 | CA | TRP | A | 389 | −36.025 | −15.753 | 12.960 | 1.00 | 86.89 | A |
| ATOM | 3113 | CB | TRP | A | 389 | −35.894 | −16.421 | 11.584 | 1.00 | 120.30 | A |
| ATOM | 3114 | CG | TRP | A | 389 | −36.839 | −15.943 | 10.529 | 1.00 | 120.30 | A |
| ATOM | 3115 | CD2 | TRP | A | 389 | −37.961 | −15.063 | 10.700 | 1.00 | 120.30 | A |
| ATOM | 3116 | CE2 | TRP | A | 389 | −38.579 | −14.928 | 9.436 | 1.00 | 120.30 | A |
| ATOM | 3117 | CE3 | TRP | A | 389 | −38.508 | −14.377 | 11.798 | 1.00 | 120.30 | A |
| ATOM | 3118 | CD1 | TRP | A | 389 | −36.825 | −16.292 | 9.207 | 1.00 | 120.30 | A |
| ATOM | 3119 | NE1 | TRP | A | 389 | −37.865 | −15.688 | 8.545 | 1.00 | 120.30 | A |
| ATOM | 3120 | CZ2 | TRP | A | 389 | −39.718 | −14.133 | 9.239 | 1.00 | 120.30 | A |
| ATOM | 3121 | CZ3 | TRP | A | 389 | −39.640 | −13.588 | 11.602 | 1.00 | 120.30 | A |
| ATOM | 3122 | CH2 | TRP | A | 389 | −40.231 | −13.474 | 10.331 | 1.00 | 120.30 | A |
| ATOM | 3123 | C | TRP | A | 389 | −35.830 | −16.834 | 14.025 | 1.00 | 86.89 | A |
| ATOM | 3124 | O | TRP | A | 389 | −35.380 | −17.935 | 13.708 | 1.00 | 86.89 | A |
| ATOM | 3125 | N | GLY | A | 390 | −36.168 | −16.535 | 15.276 | 1.00 | 67.69 | A |
| ATOM | 3126 | CA | GLY | A | 390 | −35.996 | −17.517 | 16.332 | 1.00 | 67.69 | A |
| ATOM | 3127 | C | GLY | A | 390 | −36.844 | −18.769 | 16.173 | 1.00 | 67.69 | A |
| ATOM | 3128 | O | GLY | A | 390 | −37.884 | −18.738 | 15.515 | 1.00 | 67.69 | A |
| ATOM | 3129 | N | ARG | A | 391 | −36.388 | −19.876 | 16.761 | 1.00 | 80.78 | A |
| ATOM | 3130 | CA | ARG | A | 391 | −37.113 | −21.146 | 16.708 | 1.00 | 80.78 | A |
| ATOM | 3131 | CB | ARG | A | 391 | −36.124 | −22.323 | 16.715 | 1.00 | 92.91 | A |
| ATOM | 3132 | CG | ARG | A | 391 | −35.131 | −22.323 | 15.543 | 1.00 | 92.91 | A |
| ATOM | 3133 | CD | ARG | A | 391 | −35.577 | −23.210 | 14.362 | 1.00 | 92.91 | A |
| ATOM | 3134 | NE | ARG | A | 391 | −35.085 | −24.590 | 14.458 | 1.00 | 92.91 | A |
| ATOM | 3135 | CZ | ARG | A | 391 | −35.592 | −25.535 | 15.256 | 1.00 | 92.91 | A |
| ATOM | 3136 | NH1 | ARG | A | 391 | −36.628 | −25.269 | 16.045 | 1.00 | 92.91 | A |
| ATOM | 3137 | NH2 | ARG | A | 391 | −35.054 | −26.750 | 15.271 | 1.00 | 92.91 | A |
| ATOM | 3138 | C | ARG | A | 391 | −37.996 | −21.177 | 17.955 | 1.00 | 80.78 | A |
| ATOM | 3139 | O | ARG | A | 391 | −37.832 | −20.346 | 18.844 | 1.00 | 80.78 | A |
| ATOM | 3140 | N | LYS | A | 392 | −38.938 | −22.111 | 18.022 | 1.00 | 85.60 | A |
| ATOM | 3141 | CA | LYS | A | 392 | −39.815 | −22.193 | 19.191 | 1.00 | 85.60 | A |
| ATOM | 3142 | CB | LYS | A | 392 | −41.287 | −22.188 | 18.765 | 1.00 | 115.61 | A |
| ATOM | 3143 | CG | LYS | A | 392 | −42.256 | −22.271 | 19.932 | 1.00 | 115.61 | A |
| ATOM | 3144 | CD | LYS | A | 392 | −43.683 | −22.497 | 19.467 | 1.00 | 115.61 | A |
| ATOM | 3145 | CE | LYS | A | 392 | −44.609 | −22.743 | 20.656 | 1.00 | 115.61 | A |
| ATOM | 3146 | NZ | LYS | A | 392 | −45.984 | −23.146 | 20.237 | 1.00 | 115.61 | A |
| ATOM | 3147 | C | LYS | A | 392 | −39.528 | −23.449 | 20.012 | 1.00 | 85.60 | A |
| ATOM | 3148 | O | LYS | A | 392 | −39.740 | −24.568 | 19.543 | 1.00 | 85.60 | A |
| ATOM | 3149 | N | SER | A | 393 | −39.047 | −23.257 | 21.238 | 1.00 | 52.25 | A |
| ATOM | 3150 | CA | SER | A | 393 | −38.724 | −24.374 | 22.124 | 1.00 | 52.25 | A |
| ATOM | 3151 | CB | SER | A | 393 | −37.406 | −25.035 | 21.693 | 1.00 | 62.14 | A |
| ATOM | 3152 | OG | SER | A | 393 | −37.045 | −26.085 | 22.570 | 1.00 | 62.14 | A |
| ATOM | 3153 | C | SER | A | 393 | −38.597 | −23.873 | 23.547 | 1.00 | 52.25 | A |
| ATOM | 3154 | O | SER | A | 393 | −38.370 | −22.692 | 23.758 | 1.00 | 52.25 | A |
| ATOM | 3155 | N | ASN | A | 394 | −38.763 | −24.760 | 24.523 | 1.00 | 68.20 | A |
| ATOM | 3156 | CA | ASN | A | 394 | −38.628 | −24.359 | 25.918 | 1.00 | 68.20 | A |
| ATOM | 3157 | CB | ASN | A | 394 | −39.246 | −25.398 | 26.840 | 1.00 | 71.51 | A |
| ATOM | 3158 | CG | ASN | A | 394 | −40.739 | −25.409 | 26.760 | 1.00 | 71.51 | A |
| ATOM | 3159 | OD1 | ASN | A | 394 | −41.377 | −24.363 | 26.856 | 1.00 | 71.51 | A |
| ATOM | 3160 | ND2 | ASN | A | 394 | −41.316 | −26.594 | 26.590 | 1.00 | 71.51 | A |
| ATOM | 3161 | C | ASN | A | 394 | −37.152 | −24.199 | 26.246 | 1.00 | 68.20 | A |
| ATOM | 3162 | O | ASN | A | 394 | −36.779 | −23.571 | 27.237 | 1.00 | 68.20 | A |
| ATOM | 3163 | N | TYR | A | 395 | −36.317 | −24.780 | 25.398 | 1.00 | 42.39 | A |
| ATOM | 3164 | CA | TYR | A | 395 | −34.885 | −24.705 | 25.569 | 1.00 | 42.39 | A |
| ATOM | 3165 | CB | TYR | A | 395 | −34.239 | −26.044 | 25.190 | 1.00 | 51.81 | A |
| ATOM | 3166 | CG | TYR | A | 395 | −34.626 | −27.196 | 26.075 | 1.00 | 51.81 | A |
| ATOM | 3167 | CD1 | TYR | A | 395 | −35.676 | −28.036 | 25.734 | 1.00 | 51.81 | A |
| ATOM | 3168 | CE1 | TYR | A | 395 | −36.052 | −29.098 | 26.567 | 1.00 | 51.81 | A |
| ATOM | 3169 | CD2 | TYR | A | 395 | −33.951 | −27.436 | 27.272 | 1.00 | 51.81 | A |
| ATOM | 3170 | CE2 | TYR | A | 395 | −34.318 | −28.489 | 28.111 | 1.00 | 51.81 | A |
| ATOM | 3171 | CZ | TYR | A | 395 | −35.372 | −29.314 | 27.748 | 1.00 | 51.81 | A |
| ATOM | 3172 | OH | TYR | A | 395 | −35.750 | −30.342 | 28.573 | 1.00 | 51.81 | A |
| ATOM | 3173 | C | TYR | A | 395 | −34.333 | −23.607 | 24.663 | 1.00 | 42.39 | A |
| ATOM | 3174 | O | TYR | A | 395 | −34.947 | −23.271 | 23.652 | 1.00 | 42.39 | A |
| ATOM | 3175 | N | GLU | A | 396 | −33.177 | −23.054 | 25.029 | 1.00 | 49.24 | A |
| ATOM | 3176 | CA | GLU | A | 396 | −32.517 | −22.025 | 24.232 | 1.00 | 49.24 | A |
| ATOM | 3177 | CB | GLU | A | 396 | −31.944 | −20.941 | 25.134 | 1.00 | 93.62 | A |
| ATOM | 3178 | CG | GLU | A | 396 | −32.908 | −20.415 | 26.165 | 1.00 | 93.62 | A |
| ATOM | 3179 | CD | GLU | A | 396 | −32.219 | −19.539 | 27.195 | 1.00 | 93.62 | A |
| ATOM | 3180 | OE1 | GLU | A | 396 | −31.219 | −19.998 | 27.796 | 1.00 | 93.62 | A |
| ATOM | 3181 | OE2 | GLU | A | 396 | −32.675 | −18.396 | 27.411 | 1.00 | 93.62 | A |
| ATOM | 3182 | C | GLU | A | 396 | −31.369 | −22.725 | 23.513 | 1.00 | 49.24 | A |
| ATOM | 3183 | O | GLU | A | 396 | −30.709 | −23.573 | 24.103 | 1.00 | 49.24 | A |
| ATOM | 3184 | N | PRO | A | 397 | −31.120 | −22.396 | 22.234 | 1.00 | 70.01 | A |
| ATOM | 3185 | CD | PRO | A | 397 | −31.727 | −21.359 | 21.387 | 1.00 | 42.82 | A |
| ATOM | 3186 | CA | PRO | A | 397 | −30.014 | −23.060 | 21.531 | 1.00 | 70.01 | A |
| ATOM | 3187 | CB | PRO | A | 397 | −29.940 | −22.310 | 20.205 | 1.00 | 42.82 | A |
| ATOM | 3188 | CG | PRO | A | 397 | −30.575 | −20.985 | 20.511 | 1.00 | 42.82 | A |
| ATOM | 3189 | C | PRO | A | 397 | −28.731 | −22.965 | 22.350 | 1.00 | 70.01 | A |
| ATOM | 3190 | O | PRO | A | 397 | −28.394 | −21.898 | 22.868 | 1.00 | 70.01 | A |
| ATOM | 3191 | N | GLY | A | 398 | −28.022 | −24.085 | 22.468 | 1.00 | 45.61 | A |
| ATOM | 3192 | CA | GLY | A | 398 | −26.820 | −24.107 | 23.280 | 1.00 | 45.61 | A |
| ATOM | 3193 | C | GLY | A | 398 | −27.024 | −25.100 | 24.425 | 1.00 | 45.61 | A |
| ATOM | 3194 | O | GLY | A | 398 | −26.104 | −25.842 | 24.795 | 1.00 | 45.61 | A |
| ATOM | 3195 | N | GLU | A | 399 | −28.231 | −25.113 | 24.990 | 1.00 | 45.24 | A |
| ATOM | 3196 | CA | GLU | A | 399 | −28.570 | −26.045 | 26.067 | 1.00 | 45.24 | A |
| ATOM | 3197 | CB | GLU | A | 399 | −29.898 | −25.648 | 26.739 | 1.00 | 41.49 | A |
| ATOM | 3198 | CG | GLU | A | 399 | −29.951 | −24.212 | 27.228 | 1.00 | 41.49 | A |
| ATOM | 3199 | CD | GLU | A | 399 | −31.250 | −23.839 | 27.959 | 1.00 | 41.49 | A |
| ATOM | 3200 | OE1 | GLU | A | 399 | −32.337 | −24.196 | 27.471 | 1.00 | 41.49 | A |
| ATOM | 3201 | OE2 | GLU | A | 399 | −31.196 | −23.165 | 29.016 | 1.00 | 41.49 | A |
| ATOM | 3202 | C | GLU | A | 399 | −28.726 | −27.413 | 25.402 | 1.00 | 45.24 | A |
| ATOM | 3203 | O | GLU | A | 399 | −29.330 | −27.514 | 24.333 | 1.00 | 45.24 | A |
| ATOM | 3204 | N | PRO | A | 400 | −28.174 | −28.481 | 26.006 | 1.00 | 54.38 | A |
| ATOM | 3205 | CD | PRO | A | 400 | −27.347 | −28.596 | 27.219 | 1.00 | 41.05 | A |
| ATOM | 3206 | CA | PRO | A | 400 | −28.334 | −29.785 | 25.356 | 1.00 | 49.46 | A |
| ATOM | 3207 | CB | PRO | A | 400 | −27.629 | −30.754 | 26.316 | 1.00 | 89.66 | A |
| ATOM | 3208 | CG | PRO | A | 400 | −27.585 | −30.031 | 27.616 | 1.00 | 41.05 | A |
| ATOM | 3209 | C | PRO | A | 400 | −29.784 | −30.167 | 25.054 | 1.00 | 40.21 | A |
| ATOM | 3210 | O | PRO | A | 400 | −30.047 | −30.994 | 24.173 | 1.00 | 40.42 | A |
| ATOM | 3211 | N | GLY | A | 401 | −30.727 | −29.536 | 25.746 | 1.00 | 58.91 | A |
| ATOM | 3212 | CA | GLY | A | 401 | −32.126 | −29.844 | 25.517 | 1.00 | 58.47 | A |
| ATOM | 3213 | C | GLY | A | 401 | −32.723 | −29.315 | 24.222 | 1.00 | 58.26 | A |
| ATOM | 3214 | O | GLY | A | 401 | −33.846 | −29.675 | 23.870 | 1.00 | 58.97 | A |
| ATOM | 3215 | N | PHE | A | 402 | −31.996 | −28.464 | 23.505 | 1.00 | 54.00 | A |
| ATOM | 3216 | CA | PHE | A | 402 | −32.513 | −27.915 | 22.264 | 1.00 | 54.00 | A |
| ATOM | 3217 | CB | PHE | A | 402 | −31.627 | −26.784 | 21.771 | 1.00 | 155.44 | A |
| ATOM | 3218 | CG | PHE | A | 402 | −32.323 | −25.866 | 20.833 | 1.00 | 53.77 | A |
| ATOM | 3219 | CD1 | PHE | A | 402 | −33.249 | −24.944 | 21.308 | 1.00 | 53.77 | A |
| ATOM | 3220 | CD2 | PHE | A | 402 | −32.114 | −25.960 | 19.467 | 1.00 | 53.77 | A |
| ATOM | 3221 | CE1 | PHE | A | 402 | −33.962 | −24.130 | 20.432 | 1.00 | 53.77 | A |
| ATOM | 3222 | CE2 | PHE | A | 402 | −32.823 | −25.148 | 18.581 | 1.00 | 53.77 | A |
| ATOM | 3223 | CZ | PHE | A | 402 | −33.750 | −24.233 | 19.070 | 1.00 | 53.77 | A |
| ATOM | 3224 | C | PHE | A | 402 | −32.602 | −28.997 | 21.191 | 1.00 | 54.00 | A |
| ATOM | 3225 | O | PHE | A | 402 | −31.628 | −29.711 | 20.936 | 1.00 | 54.00 | A |
| ATOM | 3226 | N | PRO | A | 403 | −33.770 | −29.120 | 20.537 | 1.00 | 72.10 | A |
| ATOM | 3227 | CD | PRO | A | 403 | −34.949 | −28.281 | 20.783 | 1.00 | 43.72 | A |
| ATOM | 3228 | CA | PRO | A | 403 | −34.057 | −30.099 | 19.481 | 1.00 | 72.10 | A |
| ATOM | 3229 | CB | PRO | A | 403 | −35.561 | −29.952 | 19.257 | 1.00 | 148.55 | A |
| ATOM | 3230 | CG | PRO | A | 403 | −36.046 | −29.243 | 20.471 | 1.00 | 43.72 | A |
| ATOM | 3231 | C | PRO | A | 403 | −33.302 | −29.854 | 18.181 | 1.00 | 72.10 | A |
| ATOM | 3232 | O | PRO | A | 403 | −33.675 | −28.982 | 17.398 | 1.00 | 72.10 | A |
| ATOM | 3233 | N | TRP | A | 404 | −32.238 | −30.609 | 17.951 | 1.00 | 60.41 | A |
| ATOM | 3234 | CA | TRP | A | 404 | −31.483 | −30.483 | 16.713 | 1.00 | 60.41 | A |
| ATOM | 3235 | CB | TRP | A | 404 | −30.011 | −30.131 | 16.978 | 1.00 | 55.04 | A |
| ATOM | 3236 | CG | TRP | A | 404 | −29.746 | −28.703 | 17.376 | 1.00 | 48.28 | A |
| ATOM | 3237 | CD2 | TRP | A | 404 | −30.001 | −27.522 | 16.600 | 1.00 | 48.28 | A |
| ATOM | 3238 | CE2 | TRP | A | 404 | −29.553 | −26.422 | 17.360 | 1.00 | 48.28 | A |
| ATOM | 3239 | CE3 | TRP | A | 404 | −30.565 | −27.288 | 15.339 | 1.00 | 48.28 | A |
| ATOM | 3240 | CD1 | TRP | A | 404 | −29.177 | −28.278 | 18.534 | 1.00 | 48.28 | A |
| ATOM | 3241 | NE1 | TRP | A | 404 | −29.056 | −26.915 | 18.535 | 1.00 | 48.28 | A |
| ATOM | 3242 | CZ2 | TRP | A | 404 | −29.649 | −25.100 | 16.903 | 1.00 | 48.28 | A |
| ATOM | 3243 | CZ3 | TRP | A | 404 | −30.664 | −25.965 | 14.883 | 1.00 | 48.28 | A |
| ATOM | 3244 | CH2 | TRP | A | 404 | −30.207 | −24.892 | 15.667 | 1.00 | 48.28 | A |
| ATOM | 3245 | C | TRP | A | 404 | −31.575 | −31.857 | 16.069 | 1.00 | 60.41 | A |
| ATOM | 3246 | O | TRP | A | 404 | −31.326 | −32.867 | 16.723 | 1.00 | 60.41 | A |
| ATOM | 3247 | N | ASN | A | 405 | −31.939 | −31.906 | 14.794 | 1.00 | 73.15 | A |
| ATOM | 3248 | CA | ASN | A | 405 | −32.045 | −33.186 | 14.122 | 1.00 | 73.18 | A |
| ATOM | 3249 | CB | ASN | A | 405 | −32.924 | −33.048 | 12.888 | 1.00 | 77.59 | A |
| ATOM | 3250 | CG | ASN | A | 405 | −33.515 | −34.359 | 12.461 | 1.00 | 77.59 | A |
| ATOM | 3251 | OD1 | ASN | A | 405 | −32.817 | −35.246 | 11.972 | 1.00 | 77.59 | A |
| ATOM | 3252 | ND2 | ASN | A | 405 | −34.811 | −34.500 | 12.655 | 1.00 | 77.59 | A |
| ATOM | 3253 | C | ASN | A | 405 | −30.653 | −33.682 | 13.745 | 1.00 | 82.12 | A |
| ATOM | 3254 | O | ASN | A | 405 | −30.229 | −33.604 | 12.583 | 1.00 | 75.18 | A |
| ATOM | 3255 | N | ILE | A | 406 | −29.935 | −34.176 | 14.750 | 1.00 | 59.66 | A |
| ATOM | 3256 | CA | ILE | A | 406 | −28.589 | −34.700 | 14.547 | 1.00 | 51.86 | A |
| ATOM | 3257 | CB | ILE | A | 406 | −27.582 | −34.072 | 15.535 | 1.00 | 57.77 | A |
| ATOM | 3258 | CG2 | ILE | A | 406 | −26.163 | −34.520 | 15.214 | 1.00 | 45.39 | A |
| ATOM | 3259 | CG1 | ILE | A | 406 | −27.646 | −32.558 | 15.427 | 1.00 | 45.39 | A |
| ATOM | 3260 | CD1 | ILE | A | 406 | −26.615 | −31.843 | 16.275 | 1.00 | 45.39 | A |
| ATOM | 3261 | C | ILE | A | 406 | −28.588 | −36.208 | 14.761 | 1.00 | 58.73 | A |
| ATOM | 3262 | O | ILE | A | 406 | −29.267 | −36.711 | 15.646 | 1.00 | 53.83 | A |
| ATOM | 3263 | N | LYS | A | 407 | −27.837 | −36.924 | 13.936 | 1.00 | 51.98 | A |
| ATOM | 3264 | CA | LYS | A | 407 | −27.724 | −38.368 | 14.053 | 1.00 | 47.58 | A |
| ATOM | 3265 | CB | LYS | A | 407 | −28.586 | −39.075 | 13.007 | 1.00 | 139.14 | A |
| ATOM | 3266 | CG | LYS | A | 407 | −30.074 | −38.831 | 13.142 | 1.00 | 125.24 | A |
| ATOM | 3267 | CD | LYS | A | 407 | −30.858 | −39.750 | 12.211 | 1.00 | 125.24 | A |
| ATOM | 3268 | CE | LYS | A | 407 | −32.348 | −39.435 | 12.227 | 1.00 | 125.24 | A |
| ATOM | 3269 | NZ | LYS | A | 407 | −32.636 | −38.085 | 11.663 | 1.00 | 125.24 | A |
| ATOM | 3270 | C | LYS | A | 407 | −26.269 | −38.660 | 13.774 | 1.00 | 56.89 | A |
| ATOM | 3271 | O | LYS | A | 407 | −25.653 | −38.002 | 12.929 | 1.00 | 55.83 | A |
| ATOM | 3272 | N | LYS | A | 408 | −25.699 | −39.616 | 14.493 | 1.00 | 59.27 | A |
| ATOM | 3273 | CA | LYS | A | 408 | −24.312 | −39.971 | 14.242 | 1.00 | 58.86 | A |
| ATOM | 3274 | CB | LYS | A | 408 | −23.758 | −40.797 | 15.397 | 1.00 | 62.76 | A |
| ATOM | 3275 | CG | LYS | A | 408 | −22.283 | −41.059 | 15.270 | 1.00 | 61.56 | A |
| ATOM | 3276 | CD | LYS | A | 408 | −21.716 | −41.624 | 16.545 | 1.00 | 61.56 | A |
| ATOM | 3277 | CE | LYS | A | 408 | −20.214 | −41.818 | 16.432 | 1.00 | 61.56 | A |
| ATOM | 3278 | NZ | LYS | A | 408 | −19.634 | −42.273 | 17.724 | 1.00 | 61.56 | A |
| ATOM | 3279 | C | LYS | A | 408 | −24.349 | −40.799 | 12.958 | 1.00 | 58.99 | A |
| ATOM | 3280 | O | LYS | A | 408 | −24.823 | −41.926 | 12.959 | 1.00 | 57.56 | A |
| ATOM | 3281 | N | ASP | A | 409 | −23.871 | −40.245 | 11.853 | 1.00 | 90.73 | A |
| ATOM | 3282 | CA | ASP | A | 409 | −23.946 | −41.000 | 10.621 | 1.00 | 90.91 | A |
| ATOM | 3283 | CB | ASP | A | 409 | −23.548 | −40.127 | 9.407 | 1.00 | 95.50 | A |
| ATOM | 3284 | CG | ASP | A | 409 | −22.283 | −39.321 | 9.633 | 1.00 | 88.32 | A |
| ATOM | 3285 | OD1 | ASP | A | 409 | −21.286 | −39.894 | 10.113 | 1.00 | 88.32 | A |
| ATOM | 3256 | OD2 | ASP | A | 409 | −22.281 | −38.117 | 9.309 | 1.00 | 88.32 | A |
| ATOM | 3287 | C | ASP | A | 409 | −23.170 | −42.318 | 10.653 | 1.00 | 90.82 | A |
| ATOM | 3288 | O | ASP | A | 409 | −22.346 | −42.554 | 11.541 | 1.00 | 94.55 | A |
| ATOM | 3289 | N | LYS | A | 410 | −23.496 | −43.173 | 9.683 | 1.00 | 142.87 | A |
| ATOM | 3290 | CA | LYS | A | 410 | −22.906 | −44.494 | 9.492 | 1.00 | 149.31 | A |
| ATOM | 3291 | CB | LYS | A | 410 | −22.853 | −44.816 | 7.987 | 1.00 | 178.05 | A |
| ATOM | 3292 | CG | LYS | A | 410 | −21.866 | −43.979 | 7.167 | 1.00 | 117.50 | A |
| ATOM | 3293 | CD | LYS | A | 410 | −22.341 | −42.544 | 6.975 | 1.00 | 117.50 | A |
| ATOM | 3294 | CE | LYS | A | 410 | −21.316 | −41.718 | 6.204 | 1.00 | 117.50 | A |
| ATOM | 3295 | NZ | LYS | A | 410 | −21.761 | −40.309 | 6.013 | 1.00 | 117.50 | A |
| ATOM | 3296 | C | LYS | A | 410 | −21.516 | −44.686 | 10.096 | 1.00 | 148.13 | A |
| ATOM | 3297 | O | LYS | A | 410 | −20.765 | −43.730 | 10.248 | 1.00 | 147.66 | A |
| ATOM | 3298 | N | ASP | A | 411 | −21.186 | −45.934 | 10.426 | 1.00 | 63.58 | A |
| ATOM | 3299 | CA | ASP | A | 411 | −19.892 | −46.304 | 11.003 | 1.00 | 61.09 | A |
| ATOM | 3300 | CB | ASP | A | 411 | −19.407 | −47.634 | 10.425 | 1.00 | 157.67 | A |
| ATOM | 3301 | CG | ASP | A | 411 | −19.899 | −48.815 | 11.219 | 1.00 | 157.67 | A |
| ATOM | 3302 | OD1 | ASP | A | 411 | −19.735 | −48.804 | 12.459 | 1.00 | 157.67 | A |
| ATOM | 3303 | OD2 | ASP | A | 411 | −20.443 | −49.756 | 10.611 | 1.00 | 157.67 | A |
| ATOM | 3304 | C | ASP | A | 411 | −18.791 | −45.277 | 10.833 | 1.00 | 62.70 | A |
| ATOM | 3305 | O | ASP | A | 411 | −17.944 | −45.397 | 9.940 | 1.00 | 66.88 | A |
| ATOM | 3306 | N | ALA | A | 412 | −18.809 | −44.272 | 11.707 | 1.00 | 78.94 | A |
| ATOM | 3307 | CA | ALA | A | 412 | −17.822 | −43.193 | 11.696 | 1.00 | 91.96 | A |
| ATOM | 3308 | CB | ALA | A | 412 | −18.022 | −42.299 | 10.477 | 1.00 | 95.81 | A |
| ATOM | 3309 | C | ALA | A | 412 | −17.945 | −42.367 | 12.968 | 1.00 | 86.66 | A |
| ATOM | 3310 | O | ALA | A | 412 | −18.876 | −42.548 | 13.748 | 1.00 | 81.71 | A |
| ATOM | 3311 | N | ASN | A | 413 | −16.999 | −41.461 | 13.174 | 1.00 | 60.96 | A |
| ATOM | 3312 | CA | ASN | A | 413 | −17.004 | −40.607 | 14.356 | 1.00 | 53.77 | A |
| ATOM | 3313 | CB | ASN | A | 413 | −15.570 | −40.238 | 14.743 | 1.00 | 71.39 | A |
| ATOM | 3314 | CG | ASN | A | 413 | −14.741 | −41.438 | 15.077 | 1.00 | 71.39 | A |
| ATOM | 3315 | OD1 | ASN | A | 413 | −15.209 | −42.347 | 15.760 | 1.00 | 71.39 | A |
| ATOM | 3316 | ND2 | ASN | A | 413 | −13.496 | −41.452 | 14.614 | 1.00 | 71.39 | A |
| ATOM | 3317 | C | ASN | A | 413 | −17.780 | −39.317 | 14.113 | 1.00 | 58.35 | A |
| ATOM | 3318 | O | ASN | A | 413 | −18.113 | −38.588 | 15.041 | 1.00 | 58.04 | A |
| ATOM | 3319 | N | THR | A | 414 | −18.060 | −39.050 | 12.848 | 1.00 | 51.95 | A |
| ATOM | 3320 | CA | THR | A | 414 | −18.733 | −37.842 | 12.435 | 1.00 | 54.99 | A |
| ATOM | 3321 | CB | THR | A | 414 | −18.362 | −37.556 | 10.972 | 1.00 | 107.11 | A |
| ATOM | 3322 | OG1 | THR | A | 414 | −19.031 | −36.377 | 10.542 | 1.00 | 77.60 | A |
| ATOM | 3323 | CG2 | THR | A | 414 | −18.737 | −38.718 | 10.074 | 1.00 | 77.60 | A |
| ATOM | 3324 | C | THR | A | 414 | −20.255 | −37.848 | 12.647 | 1.00 | 54.45 | A |
| ATOM | 3325 | O | THR | A | 414 | −20.841 | −38.892 | 12.928 | 1.00 | 55.52 | A |
| ATOM | 3326 | N | TYR | A | 415 | −20.875 | −36.668 | 12.557 | 1.00 | 42.73 | A |
| ATOM | 3327 | CA | TYR | A | 415 | −22.327 | −36.507 | 12.733 | 1.00 | 43.76 | A |
| ATOM | 3328 | CB | TYR | A | 415 | −22.658 | −35.755 | 14.021 | 1.00 | 75.45 | A |
| ATOM | 3329 | CG | TYR | A | 415 | −22.299 | −36.470 | 15.288 | 1.00 | 42.39 | A |
| ATOM | 3330 | CD1 | TYR | A | 415 | −20.966 | −36.667 | 15.646 | 1.00 | 42.39 | A |
| ATOM | 3331 | CE1 | TYR | A | 415 | −20.624 | −37.358 | 16.804 | 1.00 | 42.39 | A |
| ATOM | 3332 | CD2 | TYR | A | 415 | −23.286 | −36.976 | 16.120 | 1.00 | 42.39 | A |
| ATOM | 3333 | CE2 | TYR | A | 415 | −22.959 | −37.668 | 17.281 | 1.00 | 42.39 | A |
| ATOM | 3334 | CZ | TYR | A | 415 | −21.629 | −37.856 | 17.616 | 1.00 | 42.39 | A |
| ATOM | 3335 | OH | TYR | A | 415 | −21.303 | −38.542 | 18.768 | 1.00 | 42.39 | A |
| ATOM | 3336 | C | TYR | A | 415 | −22.947 | −35.718 | 11.586 | 1.00 | 51.32 | A |
| ATOM | 3337 | O | TYR | A | 415 | −22.247 | −35.023 | 10.843 | 1.00 | 45.97 | A |
| ATOM | 3338 | N | SER | A | 416 | −24.268 | −35.812 | 11.458 | 1.00 | 52.16 | A |
| ATOM | 3339 | CA | SER | A | 416 | −24.987 | −35.104 | 10.405 | 1.00 | 50.11 | A |
| ATOM | 3340 | CB | SER | A | 416 | −25.387 | −36.058 | 9.275 | 1.00 | 79.92 | A |
| ATOM | 3341 | OG | SER | A | 416 | −24.272 | −36.386 | 8.469 | 1.00 | 56.30 | A |
| ATOM | 3342 | C | SER | A | 416 | −26.221 | −34.427 | 10.977 | 1.00 | 56.30 | A |
| ATOM | 3343 | O | SER | A | 416 | −26.807 | −34.899 | 11.950 | 1.00 | 54.67 | A |
| ATOM | 3344 | N | LEU | A | 417 | −26.615 | −33.316 | 10.363 | 1.00 | 52.66 | A |
| ATOM | 3345 | CA | LEU | A | 417 | −27.761 | −32.545 | 10.820 | 1.00 | 51.56 | A |
| ATOM | 3346 | CB | LEU | A | 417 | −27.256 | −31.317 | 11.566 | 1.00 | 96.95 | A |
| ATOM | 3347 | CG | LEU | A | 417 | −28.191 | −30.110 | 11.561 | 1.00 | 55.06 | A |
| ATOM | 3348 | CD1 | LEU | A | 417 | −29.305 | −30.329 | 12.570 | 1.00 | 55.06 | A |
| ATOM | 3349 | CD2 | LEU | A | 417 | −27.413 | −28.858 | 11.884 | 1.00 | 55.06 | A |
| ATOM | 3350 | C | LEU | A | 417 | −28.679 | −32.092 | 9.690 | 1.00 | 63.07 | A |
| ATOM | 3351 | O | LEU | A | 417 | −28.239 | −31.876 | 8.552 | 1.00 | 58.17 | A |
| ATOM | 3352 | N | THR | A | 418 | −29.956 | −31.936 | 10.020 | 1.00 | 55.85 | A |
| ATOM | 3353 | CA | THR | A | 418 | −30.950 | −31.480 | 9.060 | 1.00 | 48.91 | A |
| ATOM | 3354 | CB | THR | A | 418 | −31.812 | −32.638 | 8.545 | 1.00 | 88.31 | A |
| ATOM | 3355 | OG1 | THR | A | 418 | −31.029 | −33.456 | 7.673 | 1.00 | 56.72 | A |
| ATOM | 3356 | CG2 | THR | A | 418 | −33.017 | −32.117 | 7.790 | 1.00 | 56.72 | A |
| ATOM | 3357 | C | THR | A | 418 | −31.862 | −30.482 | 9.739 | 1.00 | 64.10 | A |
| ATOM | 3358 | O | THR | A | 418 | −32.590 | −30.855 | 10.644 | 1.00 | 57.57 | A |
| ATOM | 3359 | N | ASP | A | 419 | −31.827 | −29.222 | 9.313 | 1.00 | 62.40 | A |
| ATOM | 3360 | CA | ASP | A | 419 | −32.677 | −28.207 | 9.923 | 1.00 | 67.51 | A |
| ATOM | 3361 | CB | ASP | A | 419 | −31.828 | −27.073 | 10.494 | 1.00 | 102.78 | A |
| ATOM | 3362 | CG | ASP | A | 419 | −32.613 | −26.192 | 11.440 | 1.00 | 80.37 | A |
| ATOM | 3363 | OD1 | ASP | A | 419 | −33.290 | −26.740 | 12.332 | 1.00 | 80.37 | A |
| ATOM | 3364 | OD2 | ASP | A | 419 | −32.559 | −24.956 | 11.305 | 1.00 | 80.37 | A |
| ATOM | 3365 | C | ASP | A | 419 | −33.677 | −27.656 | 8.920 | 1.00 | 71.60 | A |
| ATOM | 3366 | O | ASP | A | 419 | −33.297 | −27.158 | 7.863 | 1.00 | 66.49 | A |
| ATOM | 3367 | N | LEU | A | 420 | −34.958 | −27.740 | 9.262 | 1.00 | 63.03 | A |
| ATOM | 3368 | CA | LEU | A | 420 | −36.033 | −27.281 | 8.381 | 1.00 | 67.72 | A |
| ATOM | 3369 | CB | LEU | A | 420 | −37.122 | −28.356 | 8.317 | 1.00 | 124.20 | A |
| ATOM | 3370 | CG | LEU | A | 420 | −38.081 | −28.334 | 7.127 | 1.00 | 83.15 | A |
| ATOM | 3371 | CD1 | LEU | A | 420 | −37.295 | −28.632 | 5.857 | 1.00 | 83.15 | A |
| ATOM | 3372 | CD2 | LEU | A | 420 | −39.182 | −29.360 | 7.325 | 1.00 | 83.15 | A |
| ATOM | 3373 | C | LEU | A | 420 | −36.646 | −25.952 | 8.834 | 1.00 | 70.29 | A |
| ATOM | 3374 | O | LEU | A | 420 | −36.613 | −25.623 | 10.015 | 1.00 | 69.61 | A |
| ATOM | 3375 | N | VAL | A | 421 | −37.211 | −25.194 | 7.897 | 1.00 | 79.01 | A |
| ATOM | 3376 | CA | VAL | A | 421 | −37.820 | −23.906 | 8.222 | 1.00 | 77.52 | A |
| ATOM | 3377 | CB | VAL | A | 421 | −36.820 | −22.750 | 8.020 | 1.00 | 142.04 | A |
| ATOM | 3378 | CG1 | VAL | A | 421 | −37.510 | −21.411 | 8.210 | 1.00 | 97.47 | A |
| ATOM | 3379 | CG2 | VAL | A | 421 | −35.700 | −22.880 | 9.008 | 1.00 | 97.47 | A |
| ATOM | 3380 | C | VAL | A | 421 | −39.058 | −23.618 | 7.383 | 1.00 | 79.78 | A |
| ATOM | 3381 | O | VAL | A | 421 | −39.064 | −23.824 | 6.166 | 1.00 | 75.38 | A |
| ATOM | 3382 | N | ARG | A | 422 | −40.100 | −23.127 | 8.046 | 1.00 | 75.54 | A |
| ATOM | 3383 | CA | ARG | A | 422 | −41.365 | −22.795 | 7.394 | 1.00 | 77.67 | A |
| ATOM | 3384 | CB | ARG | A | 422 | −42.487 | −22.809 | 8.433 | 1.00 | 180.95 | A |
| ATOM | 3385 | CG | ARG | A | 422 | −43.653 | −23.718 | 8.107 | 1.00 | 141.54 | A |
| ATOM | 3386 | CD | ARG | A | 422 | −44.588 | −23.096 | 7.093 | 1.00 | 141.54 | A |
| ATOM | 3387 | NE | ARG | A | 422 | −45.780 | −23.918 | 6.908 | 1.00 | 141.54 | A |
| ATOM | 3388 | CZ | ARG | A | 422 | −46.839 | −23.558 | 6.190 | 1.00 | 141.54 | A |
| ATOM | 3389 | NH1 | ARG | A | 422 | −46.860 | −22.379 | 5.579 | 1.00 | 141.54 | A |
| ATOM | 3390 | NH2 | ARG | A | 422 | −47.880 | −24.374 | 6.085 | 1.00 | 141.54 | A |
| ATOM | 3391 | C | ARG | A | 422 | −41.273 | −21.409 | 6.759 | 1.00 | 74.90 | A |
| ATOM | 3392 | O | ARG | A | 422 | −41.396 | −20.402 | 7.451 | 1.00 | 65.57 | A |
| ATOM | 3393 | N | THR | A | 423 | −41.065 | −21.359 | 5.446 | 1.00 | 113.19 | A |
| ATOM | 3394 | CA | THR | A | 423 | −40.949 | −20.084 | 4.739 | 1.00 | 122.84 | A |
| ATOM | 3395 | CB | THR | A | 423 | −40.407 | −20.277 | 3.294 | 1.00 | 200.84 | A |
| ATOM | 3396 | OG1 | THR | A | 423 | −39.152 | −20.966 | 3.337 | 1.00 | 164.51 | A |
| ATOM | 3397 | CG2 | THR | A | 423 | −40.197 | −18.926 | 2.609 | 1.00 | 164.51 | A |
| ATOM | 3398 | C | THR | A | 423 | −42.282 | −19.351 | 4.648 | 1.00 | 121.06 | A |
| ATOM | 3399 | O | THR | A | 423 | −42.799 | −19.146 | 3.555 | 1.00 | 119.55 | A |
| ATOM | 3400 | N | THR | A | 424 | −42.841 | −18.958 | 5.790 | 1.00 | 166.52 | A |
| ATOM | 3401 | CA | THR | A | 424 | −44.109 | −18.237 | 5.790 | 1.00 | 166.52 | A |
| ATOM | 3402 | CB | THR | A | 424 | −44.612 | −17.976 | 7.221 | 1.00 | 180.67 | A |
| ATOM | 3403 | OG1 | THR | A | 424 | −44.737 | −19.225 | 7.913 | 1.00 | 126.48 | A |
| ATOM | 3404 | CG2 | THR | A | 424 | −45.971 | −17.279 | 7.194 | 1.00 | 126.48 | A |
| ATOM | 3405 | C | THR | A | 424 | −43.913 | −16.905 | 5.075 | 1.00 | 166.52 | A |
| ATOM | 3406 | O | THR | A | 424 | −42.797 | −16.388 | 5.008 | 1.00 | 166.52 | A |
| ATOM | 3407 | N | SER | A | 425 | −44.996 | −16.350 | 4.544 | 1.00 | 139.15 | A |
| ATOM | 3408 | CA | SER | A | 425 | −44.922 | −15.091 | 3.815 | 1.00 | 139.15 | A |
| ATOM | 3409 | CB | SER | A | 425 | −46.191 | −14.892 | 2.989 | 1.00 | 183.53 | A |
| ATOM | 3410 | OG | SER | A | 425 | −46.081 | −13.732 | 2.181 | 1.00 | 79.47 | A |
| ATOM | 3411 | C | SER | A | 425 | −44.698 | −13.870 | 4.701 | 1.00 | 139.15 | A |
| ATOM | 3412 | O | SER | A | 425 | −44.599 | −12.749 | 4.204 | 1.00 | 139.15 | A |
| ATOM | 3413 | N | LYS | A | 426 | −44.617 | −14.086 | 6.010 | 1.00 | 169.46 | A |
| ATOM | 3414 | CA | LYS | A | 426 | −44.403 | −12.991 | 6.953 | 1.00 | 169.46 | A |
| ATOM | 3415 | CB | LYS | A | 426 | −44.306 | −13.536 | 8.377 | 1.00 | 184.02 | A |
| ATOM | 3416 | CG | LYS | A | 426 | −45.448 | −14.443 | 8.779 | 1.00 | 128.32 | A |
| ATOM | 3417 | CD | LYS | A | 426 | −45.174 | −15.072 | 10.128 | 1.00 | 128.32 | A |
| ATOM | 3418 | CE | LYS | A | 426 | −46.235 | −16.092 | 10.485 | 1.00 | 128.32 | A |
| ATOM | 3419 | NZ | LYS | A | 426 | −45.936 | −16.742 | 11.790 | 1.00 | 128.32 | A |
| ATOM | 3420 | C | LYS | A | 426 | −43.121 | −12.225 | 6.632 | 1.00 | 169.46 | A |
| ATOM | 3421 | O | LYS | A | 426 | −43.075 | −10.998 | 6.735 | 1.00 | 169.46 | A |
| ATOM | 3422 | N | MET | A | 427 | −42.082 | −12.963 | 6.247 | 1.00 | 171.16 | A |
| ATOM | 3423 | CA | MET | A | 427 | −40.785 | −12.380 | 5.921 | 1.00 | 171.16 | A |
| ATOM | 3424 | CB | MET | A | 427 | −39.751 | −13.488 | 5.725 | 1.00 | 168.17 | A |
| ATOM | 3425 | CG | MET | A | 427 | −40.177 | −14.559 | 4.743 | 1.00 | 168.17 | A |
| ATOM | 3426 | SD | MET | A | 427 | −38.937 | −15.846 | 4.601 | 1.00 | 168.17 | A |
| ATOM | 3427 | CE | MET | A | 427 | −39.258 | −16.799 | 6.088 | 1.00 | 168.17 | A |
| ATOM | 3428 | C | MET | A | 427 | −40.816 | −11.491 | 4.686 | 1.00 | 171.16 | A |
| ATOM | 3429 | O | MET | A | 427 | −41.879 | −11.043 | 4.257 | 1.00 | 171.16 | A |
| ATOM | 3430 | N | SER | A | 428 | −39.640 | −11.240 | 4.117 | 1.00 | 132.92 | A |
| ATOM | 3431 | CA | SER | A | 428 | −39.533 | −10.395 | 2.935 | 1.00 | 134.57 | A |
| ATOM | 3432 | CB | SER | A | 428 | −39.875 | −8.947 | 3.299 | 1.00 | 98.17 | A |
| ATOM | 3433 | OG | SER | A | 428 | −38.959 | −8.432 | 4.253 | 1.00 | 94.88 | A |
| ATOM | 3434 | C | SER | A | 428 | −38.146 | −10.433 | 2.298 | 1.00 | 130.52 | A |
| ATOM | 3435 | O | SER | A | 428 | −37.714 | −9.445 | 1.702 | 1.00 | 130.40 | A |
| ATOM | 3436 | N | SER | A | 429 | −37.450 | −11.562 | 2.418 | 1.00 | 145.26 | A |
| ATOM | 3437 | CA | SER | A | 429 | −36.111 | −11.689 | 1.841 | 1.00 | 143.93 | A |
| ATOM | 3438 | CB | SER | A | 429 | −35.075 | −10.983 | 2.723 | 1.00 | 79.27 | A |
| ATOM | 3439 | OG | SER | A | 429 | −35.305 | −9.591 | 2.786 | 1.00 | 79.27 | A |
| ATOM | 3440 | C | SER | A | 429 | −35.672 | −13.132 | 1.655 | 1.00 | 142.31 | A |
| ATOM | 3441 | O | SER | A | 429 | −34.645 | −13.400 | 1.035 | 1.00 | 131.01 | A |
| ATOM | 3442 | N | GLN | A | 430 | −36.452 | −14.061 | 2.186 | 1.00 | 80.04 | A |
| ATOM | 3443 | CA | GLN | A | 430 | −36.108 | −15.474 | 2.100 | 1.00 | 80.04 | A |
| ATOM | 3444 | CB | GLN | A | 430 | −35.880 | −15.917 | 0.658 | 1.00 | 184.25 | A |
| ATOM | 3445 | CG | GLN | A | 430 | −35.577 | −17.397 | 0.563 | 1.00 | 113.24 | A |
| ATOM | 3446 | CD | GLN | A | 430 | −35.303 | −17.852 | −0.847 | 1.00 | 113.24 | A |
| ATOM | 3447 | OE1 | GLN | A | 430 | −34.276 | −17.510 | −1.437 | 1.00 | 113.24 | A |
| ATOM | 3448 | NE2 | GLN | A | 430 | −36.225 | −18.628 | −1.403 | 1.00 | 113.24 | A |
| ATOM | 3449 | C | GLN | A | 430 | −34.832 | −15.683 | 2.894 | 1.00 | 80.04 | A |
| ATOM | 3450 | O | GLN | A | 430 | −33.762 | −15.220 | 2.502 | 1.00 | 80.04 | A |
| ATOM | 3451 | N | PRO | A | 431 | −34.929 | −16.403 | 4.019 | 1.00 | 65.69 | A |
| ATOM | 3452 | CD | PRO | A | 431 | −36.045 | −17.259 | 4.457 | 1.00 | 68.90 | A |
| ATOM | 3453 | CA | PRO | A | 431 | −33.753 | −16.642 | 4.846 | 1.00 | 65.68 | A |
| ATOM | 3454 | CB | PRO | A | 431 | −34.316 | −17.451 | 6.006 | 1.00 | 167.86 | A |
| ATOM | 3455 | CG | PRO | A | 431 | −35.347 | −18.276 | 5.335 | 1.00 | 138.42 | A |
| ATOM | 3456 | C | PRO | A | 431 | −32.631 | −17.372 | 4.134 | 1.00 | 62.30 | A |
| ATOM | 3457 | O | PRO | A | 431 | −32.838 | −18.058 | 3.127 | 1.00 | 62.18 | A |
| ATOM | 3458 | N | VAL | A | 432 | −31.438 | −17.179 | 4.678 | 1.00 | 56.06 | A |
| ATOM | 3459 | CA | VAL | A | 432 | −30.227 | −17.807 | 4.206 | 1.00 | 58.44 | A |
| ATOM | 3460 | CB | VAL | A | 432 | −29.194 | −16.754 | 3.812 | 1.00 | 88.16 | A |
| ATOM | 3461 | CG1 | VAL | A | 432 | −28.845 | −15.885 | 4.997 | 1.00 | 60.24 | A |
| ATOM | 3462 | CG2 | VAL | A | 432 | −27.978 | −17.427 | 3.287 | 1.00 | 60.24 | A |
| ATOM | 3463 | C | VAL | A | 432 | −29.772 | −18.599 | 5.442 | 1.00 | 56.44 | A |
| ATOM | 3464 | O | VAL | A | 432 | −30.049 | −18.207 | 6.578 | 1.00 | 46.80 | A |
| ATOM | 3465 | N | CYS | A | 433 | −29.105 | −19.723 | 5.241 | 1.00 | 64.22 | A |
| ATOM | 3466 | CA | CYS | A | 433 | −28.689 | −20.523 | 6.379 | 1.00 | 60.10 | A |
| ATOM | 3467 | C | CYS | A | 433 | −27.267 | −20.205 | 6.832 | 1.00 | 57.74 | A |
| ATOM | 3468 | O | CYS | A | 433 | −26.324 | −20.287 | 6.042 | 1.00 | 58.48 | A |
| ATOM | 3469 | CB | CYS | A | 433 | −28.800 | −22.013 | 6.032 | 1.00 | 153.66 | A |
| ATOM | 3470 | SG | CYS | A | 433 | −28.469 | −23.135 | 7.429 | 1.00 | 100.77 | A |
| ATOM | 3471 | N | VAL | A | 434 | −27.118 | −19.826 | 8.103 | 1.00 | 46.80 | A |
| ATOM | 3472 | CA | VAL | A | 434 | −25.800 | −19.544 | 8.669 | 1.00 | 42.79 | A |
| ATOM | 3473 | CB | VAL | A | 434 | −25.765 | −18.233 | 9.468 | 1.00 | 54.96 | A |
| ATOM | 3474 | CG1 | VAL | A | 434 | −24.331 | −17.905 | 9.827 | 1.00 | 36.74 | A |
| ATOM | 3475 | CG2 | VAL | A | 434 | −26.375 | −17.097 | 8.653 | 1.00 | 36.74 | A |
| ATOM | 3476 | C | VAL | A | 434 | −25.457 | −20.693 | 9.613 | 1.00 | 53.19 | A |
| ATOM | 3477 | O | VAL | A | 434 | −26.154 | −20.921 | 10.600 | 1.00 | 44.25 | A |
| ATOM | 3478 | N | VAL | A | 435 | −24.382 | −21.409 | 9.298 | 1.00 | 40.99 | A |
| ATOM | 3479 | CA | VAL | A | 435 | −23.939 | −22.558 | 10.082 | 1.00 | 32.73 | A |
| ATOM | 3480 | CB | VAL | A | 435 | −23.565 | −23.710 | 9.146 | 1.00 | 58.12 | A |
| ATOM | 3481 | CG1 | VAL | A | 435 | −22.868 | −24.809 | 9.909 | 1.00 | 37.45 | A |
| ATOM | 3482 | CG2 | VAL | A | 435 | −24.816 | −24.229 | 8.465 | 1.00 | 37.45 | A |
| ATOM | 3483 | C | VAL | A | 435 | −22.753 | −22.279 | 10.985 | 1.00 | 40.65 | A |
| ATOM | 3484 | O | VAL | A | 435 | −21.691 | −21.882 | 10.506 | 1.00 | 41.24 | A |
| ATOM | 3485 | N | PHE | A | 436 | −22.937 | −22.497 | 12.289 | 1.00 | 40.37 | A |
| ATOM | 3486 | CA | PHE | A | 436 | −21.868 | −22.282 | 13.259 | 1.00 | 36.57 | A |
| ATOM | 3487 | CB | PHE | A | 436 | −22.389 | −21.531 | 14.469 | 1.00 | 64.77 | A |
| ATOM | 3488 | CG | PHE | A | 436 | −22.863 | −20.154 | 14.159 | 1.00 | 39.54 | A |
| ATOM | 3489 | CD1 | PHE | A | 436 | −24.172 | −19.929 | 13.764 | 1.00 | 39.54 | A |
| ATOM | 3490 | CD2 | PHE | A | 436 | −21.996 | −19.065 | 14.268 | 1.00 | 39.54 | A |
| ATOM | 3491 | CE1 | PHE | A | 436 | −24.618 | −18.638 | 13.483 | 1.00 | 39.54 | A |
| ATOM | 3492 | CE2 | PHE | A | 436 | −22.433 | −17.760 | 13.987 | 1.00 | 39.54 | A |
| ATOM | 3493 | CZ | PHE | A | 436 | −23.746 | −17.548 | 13.595 | 1.00 | 39.54 | A |
| ATOM | 3494 | C | PHE | A | 436 | −21.279 | −23.608 | 13.698 | 1.00 | 44.40 | A |
| ATOM | 3495 | O | PHE | A | 436 | −21.970 | −24.448 | 14.259 | 1.00 | 43.78 | A |
| ATOM | 3496 | N | HIS | A | 437 | −19.994 | −23.791 | 13.430 | 1.00 | 38.97 | A |
| ATOM | 3497 | CA | HIS | A | 437 | −19.295 | −25.015 | 13.797 | 1.00 | 40.04 | A |
| ATOM | 3498 | CB | HIS | A | 437 | −19.457 | −26.040 | 12.688 | 1.00 | 66.20 | A |
| ATOM | 3499 | CG | HIS | A | 437 | −18.805 | −27.345 | 12.983 | 1.00 | 42.57 | A |
| ATOM | 3500 | CD2 | HIS | A | 437 | −17.609 | −27.847 | 12.604 | 1.00 | 42.57 | A |
| ATOM | 3501 | ND1 | HIS | A | 437 | −19.376 | −28.291 | 13.800 | 1.00 | 42.57 | A |
| ATOM | 3502 | CE1 | HIS | A | 437 | −18.560 | −29.321 | 13.914 | 1.00 | 42.57 | A |
| ATOM | 3503 | NE2 | HIS | A | 437 | −17.480 | −29.078 | 13.198 | 1.00 | 42.57 | A |
| ATOM | 3504 | C | HIS | A | 437 | −17.807 | −24.722 | 14.024 | 1.00 | 44.35 | A |
| ATOM | 3505 | O | HIS | A | 437 | −17.302 | −23.702 | 13.563 | 1.00 | 46.57 | A |
| ATOM | 3506 | N | ASP | A | 438 | −17.107 | −25.603 | 14.733 | 1.00 | 49.89 | A |
| ATOM | 3507 | CA | ASP | A | 438 | −15.686 | −25.393 | 15.008 | 1.00 | 51.80 | A |
| ATOM | 3508 | CB | ASP | A | 438 | −15.130 | −26.561 | 15.820 | 1.00 | 99.38 | A |
| ATOM | 3509 | CG | ASP | A | 438 | −15.370 | −26.393 | 17.314 | 1.00 | 70.34 | A |
| ATOM | 3510 | OD1 | ASP | A | 438 | −16.297 | −25.632 | 17.681 | 1.00 | 70.34 | A |
| ATOM | 3511 | OD2 | ASP | A | 438 | −14.635 | −27.026 | 18.113 | 1.00 | 70.34 | A |
| ATOM | 3512 | C | ASP | A | 438 | −14.886 | −25.178 | 13.731 | 1.00 | 53.14 | A |
| ATOM | 3513 | O | ASP | A | 438 | −14.058 | −24.273 | 13.652 | 1.00 | 56.70 | A |
| ATOM | 3514 | N | THR | A | 439 | −15.121 | −26.016 | 12.733 | 1.00 | 68.28 | A |
| ATOM | 3515 | CA | THR | A | 439 | −14.460 | −25.849 | 11.448 | 1.00 | 65.63 | A |
| ATOM | 3516 | CB | THR | A | 439 | −14.530 | −27.152 | 10.627 | 1.00 | 45.93 | A |
| ATOM | 3517 | OG1 | THR | A | 439 | −15.892 | −27.475 | 10.324 | 1.00 | 31.74 | A |
| ATOM | 3518 | CG2 | THR | A | 439 | −13.928 | −28.304 | 11.436 | 1.00 | 31.74 | A |
| ATOM | 3519 | C | THR | A | 439 | −15.350 | −24.770 | 10.846 | 1.00 | 71.40 | A |
| ATOM | 3520 | O | THR | A | 439 | −16.542 | −24.765 | 11.116 | 1.00 | 71.74 | A |
| ATOM | 3521 | N | LEU | A | 440 | −14.805 | −23.858 | 10.052 | 1.00 | 58.91 | A |
| ATOM | 3522 | CA | LEU | A | 440 | −15.608 | −22.751 | 9.498 | 1.00 | 60.93 | A |
| ATOM | 3523 | CB | LEU | A | 440 | −16.609 | −23.236 | 8.412 | 1.00 | 82.18 | A |
| ATOM | 3524 | CG | LEU | A | 440 | −17.824 | −24.212 | 8.366 | 1.00 | 62.18 | A |
| ATOM | 3525 | CD1 | LEU | A | 440 | −17.339 | −25.622 | 8.529 | 1.00 | 62.18 | A |
| ATOM | 3526 | CD2 | LEU | A | 440 | −18.898 | −23.887 | 9.379 | 1.00 | 62.18 | A |
| ATOM | 3527 | C | LEU | A | 440 | −16.365 | −21.970 | 10.576 | 1.00 | 70.88 | A |
| ATOM | 3528 | O | LEU | A | 440 | −17.567 | −22.124 | 10.711 | 1.00 | 69.10 | A |
| ATOM | 3529 | N | GLU | A | 441 | −15.660 | −21.127 | 11.334 | 1.00 | 68.07 | A |
| ATOM | 3530 | CA | GLU | A | 441 | −16.267 | −20.330 | 12.414 | 1.00 | 68.07 | A |
| ATOM | 3531 | CB | GLU | A | 441 | −15.457 | −19.051 | 12.643 | 1.00 | 197.78 | A |
| ATOM | 3532 | CG | GLU | A | 441 | −14.041 | −19.303 | 13.148 | 1.00 | 121.14 | A |
| ATOM | 3533 | CD | GLU | A | 441 | −13.312 | −18.018 | 13.522 | 1.00 | 121.14 | A |
| ATOM | 3534 | OE1 | GLU | A | 441 | −13.175 | −17.129 | 12.651 | 1.00 | 121.14 | A |
| ATOM | 3535 | OE2 | GLU | A | 441 | −12.875 | −17.898 | 14.689 | 1.00 | 121.14 | A |
| ATOM | 3536 | C | GLU | A | 441 | −17.750 | −19.984 | 12.201 | 1.00 | 68.07 | A |
| ATOM | 3537 | O | GLU | A | 441 | −18.567 | −20.106 | 13.112 | 1.00 | 68.07 | A |
| ATOM | 3538 | N | ALA | A | 442 | −18.097 | −19.555 | 10.997 | 1.00 | 68.16 | A |
| ATOM | 3539 | CA | ALA | A | 442 | −19.484 | −19.237 | 10.682 | 1.00 | 66.38 | A |
| ATOM | 3540 | CB | ALA | A | 442 | −19.948 | −18.044 | 11.482 | 1.00 | 35.77 | A |
| ATOM | 3541 | C | ALA | A | 442 | −19.624 | −18.955 | 9.193 | 1.00 | 62.37 | A |
| ATOM | 3542 | O | ALA | A | 442 | −19.168 | −17.920 | 8.707 | 1.00 | 59.78 | A |
| ATOM | 3543 | N | GLN | A | 443 | −20.244 | −19.885 | 8.465 | 1.00 | 34.04 | A |
| ATOM | 3544 | CA | GLN | A | 443 | −20.446 | −19.730 | 7.025 | 1.00 | 37.32 | A |
| ATOM | 3545 | CB | GLN | A | 443 | −19.868 | −20.907 | 6.268 | 1.00 | 76.57 | A |
| ATOM | 3546 | CG | GLN | A | 443 | −18.440 | −20.707 | 5.866 | 1.00 | 59.80 | A |
| ATOM | 3547 | CD | GLN | A | 443 | −18.043 | −21.629 | 4.730 | 1.00 | 59.80 | A |
| ATOM | 3548 | OE1 | GLN | A | 443 | −18.742 | −21.709 | 3.709 | 1.00 | 59.80 | A |
| ATOM | 3549 | NE2 | GLN | A | 443 | −16.917 | −22.331 | 4.891 | 1.00 | 59.80 | A |
| ATOM | 3550 | C | GLN | A | 443 | −21.906 | −19.562 | 6.625 | 1.00 | 39.49 | A |
| ATOM | 3551 | O | GLN | A | 443 | −22.807 | −20.112 | 7.260 | 1.00 | 40.55 | A |
| ATOM | 3552 | N | VAL | A | 444 | −22.134 | −18.791 | 5.563 | 1.00 | 53.18 | A |
| ATOM | 3553 | CA | VAL | A | 444 | −23.486 | −18.546 | 5.082 | 1.00 | 50.33 | A |
| ATOM | 3554 | CB | VAL | A | 444 | −23.680 | −17.067 | 4.729 | 1.00 | 67.80 | A |
| ATOM | 3555 | CG1 | VAL | A | 444 | −25.156 | −16.753 | 4.625 | 1.00 | 60.10 | A |
| ATOM | 3556 | CG2 | VAL | A | 444 | −23.024 | −16.192 | 5.782 | 1.00 | 60.10 | A |
| ATOM | 3557 | C | VAL | A | 444 | −23.760 | −19.411 | 3.853 | 1.00 | 54.15 | A |
| ATOM | 3558 | O | VAL | A | 444 | −22.875 | −19.623 | 3.018 | 1.00 | 56.41 | A |
| ATOM | 3559 | N | TYR | A | 445 | −24.979 | −19.940 | 3.763 | 1.00 | 44.77 | A |
| ATOM | 3560 | CA | TYR | A | 445 | −25.375 | −20.798 | 2.642 | 1.00 | 43.84 | A |
| ATOM | 3561 | CB | TYR | A | 445 | −25.375 | −22.261 | 3.068 | 1.00 | 78.99 | A |
| ATOM | 3562 | CG | TYR | A | 445 | −24.033 | −22.737 | 3.562 | 1.00 | 51.41 | A |
| ATOM | 3563 | CD1 | TYR | A | 445 | −23.690 | −22.654 | 4.915 | 1.00 | 51.41 | A |
| ATOM | 3564 | CE1 | TYR | A | 445 | −22.437 | −23.100 | 5.380 | 1.00 | 51.41 | A |
| ATOM | 3565 | CD2 | TYR | A | 445 | −23.095 | −23.269 | 2.680 | 1.00 | 51.41 | A |
| ATOM | 3566 | CE2 | TYR | A | 445 | −21.845 | −23.711 | 3.128 | 1.00 | 51.41 | A |
| ATOM | 3567 | CZ | TYR | A | 445 | −21.526 | −23.627 | 4.479 | 1.00 | 51.41 | A |
| ATOM | 3568 | OH | TYR | A | 445 | −20.316 | −24.095 | 4.924 | 1.00 | 51.41 | A |
| ATOM | 3569 | C | TYR | A | 445 | −26.753 | −20.400 | 2.131 | 1.00 | 45.30 | A |
| ATOM | 3570 | O | TYR | A | 445 | −27.671 | −20.145 | 2.913 | 1.00 | 48.16 | A |
| ATOM | 3571 | N | THR | A | 446 | −26.907 | −20.378 | 0.811 | 1.00 | 39.37 | A |
| ATOM | 3572 | CA | THR | A | 446 | −28.167 | −19.931 | 0.219 | 1.00 | 46.35 | A |
| ATOM | 3573 | CB | THR | A | 446 | −27.921 | −18.690 | −0.660 | 1.00 | 65.48 | A |
| ATOM | 3574 | OG1 | THR | A | 446 | −26.950 | −19.015 | −1.661 | 1.00 | 53.63 | A |
| ATOM | 3575 | CG2 | THR | A | 446 | −27.402 | −17.530 | 0.167 | 1.00 | 53.63 | A |
| ATOM | 3576 | C | THR | A | 446 | −28.962 | −20.913 | −0.626 | 1.00 | 56.15 | A |
| ATOM | 3577 | O | THR | A | 446 | −28.851 | −22.129 | −0.488 | 1.00 | 59.82 | A |
| ATOM | 3578 | N | CYS | A | 447 | −29.771 | −20.310 | −1.497 | 1.00 | 83.35 | A |
| ATOM | 3579 | CA | CYS | A | 447 | −30.663 | −20.928 | −2.490 | 1.00 | 84.33 | A |
| ATOM | 3580 | C | CYS | A | 447 | −31.835 | −21.800 | −2.062 | 1.00 | 84.36 | A |
| ATOM | 3581 | O | CYS | A | 447 | −31.756 | −22.585 | −1.126 | 1.00 | 81.22 | A |
| ATOM | 3582 | CB | CYS | A | 447 | −29.854 | −21.658 | −3.576 | 1.00 | 126.13 | A |
| ATOM | 3583 | SG | CYS | A | 447 | −28.797 | −23.014 | −3.007 | 1.00 | 111.66 | A |
| ATOM | 3584 | N | SER | A | 448 | −32.939 | −21.617 | −2.785 | 1.00 | 200.84 | A |
| ATOM | 3585 | CA | SER | A | 448 | −34.181 | −22.343 | −2.559 | 1.00 | 200.84 | A |
| ATOM | 3586 | CB | SER | A | 448 | −35.265 | −21.393 | −2.049 | 1.00 | 200.84 | A |
| ATOM | 3587 | OG | SER | A | 448 | −34.863 | −20.775 | −0.840 | 1.00 | 200.84 | A |
| ATOM | 3588 | C | SER | A | 448 | −34.632 | −22.990 | −3.867 | 1.00 | 200.84 | A |
| ATOM | 3589 | O | SER | A | 448 | −33.805 | −23.252 | −4.742 | 1.00 | 200.84 | A |
| ATOM | 3590 | N | GLU | A | 449 | −35.939 | −23.221 | −4.009 | 1.00 | 96.88 | A |
| ATOM | 3591 | CA | GLU | A | 449 | −36.475 | −23.880 | −5.202 | 1.00 | 96.88 | A |
| ATOM | 3592 | CB | GLU | A | 449 | −35.970 | −23.198 | −6.482 | 1.00 | 121.21 | A |
| ATOM | 3593 | CG | GLU | A | 449 | −36.712 | −21.927 | −6.856 | 1.00 | 121.21 | A |
| ATOM | 3594 | CD | GLU | A | 449 | −36.910 | −21.000 | −5.673 | 1.00 | 121.21 | A |
| ATOM | 3595 | OE1 | GLU | A | 449 | −35.908 | −20.645 | −5.019 | 1.00 | 121.21 | A |
| ATOM | 3596 | OE2 | GLU | A | 449 | −38.069 | −20.626 | −5.398 | 1.00 | 121.21 | A |
| ATOM | 3597 | C | GLU | A | 449 | −35.976 | −25.320 | −5.160 | 1.00 | 96.88 | A |
| ATOM | 3598 | O | GLU | A | 449 | −36.711 | −26.259 | −5.432 | 1.00 | 96.88 | A |
| ATOM | 3599 | N | GLY | A | 450 | −34.711 | −25.467 | −4.789 | 1.00 | 61.63 | A |
| ATOM | 3600 | CA | GLY | A | 450 | −34.063 | −26.756 | −4.695 | 1.00 | 61.63 | A |
| ATOM | 3601 | C | GLY | A | 450 | −32.618 | −26.504 | −5.060 | 1.00 | 61.63 | A |
| ATOM | 3602 | O | GLY | A | 450 | −31.843 | −27.436 | −5.266 | 1.00 | 61.63 | A |
| ATOM | 3603 | N | CYS | A | 451 | −32.272 | −25.221 | −5.133 | 1.00 | 198.68 | A |
| ATOM | 3604 | CA | CYS | A | 451 | −30.934 | −24.791 | −5.497 | 1.00 | 198.68 | A |
| ATOM | 3605 | CB | CYS | A | 451 | −29.900 | −25.348 | −4.550 | 1.00 | 136.57 | A |
| ATOM | 3606 | SG | CYS | A | 451 | −30.053 | −24.597 | −2.924 | 1.00 | 136.57 | A |
| ATOM | 3607 | C | CYS | A | 451 | −30.650 | −25.309 | −6.856 | 1.00 | 198.68 | A |
| ATOM | 3608 | O | CYS | A | 451 | −29.637 | −26.042 | −6.985 | 1.00 | 198.68 | A |
| ATOM | 3609 | OXT | CYS | A | 451 | −31.454 | −24.969 | −7.751 | 1.00 | 108.94 | A |
| ATOM | 3610 | CB | ARG | B | 1 | −12.181 | 20.412 | 42.960 | 1.00 | 144.65 | B |
| ATOM | 3611 | CG | ARG | B | 1 | −11.150 | 19.470 | 43.601 | 1.00 | 144.65 | B |
| ATOM | 3612 | CD | ARG | B | 1 | −11.061 | 19.589 | 45.123 | 1.00 | 144.65 | B |
| ATOM | 3613 | NE | ARG | B | 1 | −11.936 | 18.648 | 45.827 | 1.00 | 144.65 | B |
| ATOM | 3614 | CZ | ARG | B | 1 | −11.808 | 17.321 | 45.795 | 1.00 | 144.65 | B |
| ATOM | 3615 | NH1 | ARG | B | 1 | −10.838 | 16.754 | 45.091 | 1.00 | 144.65 | B |
| ATOM | 3616 | NH2 | ARG | B | 1 | −12.656 | 16.556 | 46.468 | 1.00 | 144.65 | B |
| ATOM | 3617 | C | ARG | B | 1 | −14.068 | 18.754 | 43.190 | 1.00 | 197.14 | B |
| ATOM | 3618 | O | ARG | B | 1 | −13.658 | 17.869 | 43.940 | 1.00 | 197.14 | B |
| ATOM | 3619 | N | ARG | B | 1 | −13.815 | 20.630 | 44.809 | 1.00 | 197.14 | B |
| ATOM | 3620 | CA | ARG | B | 1 | −13.643 | 20.212 | 43.390 | 1.00 | 197.14 | B |
| ATOM | 3621 | N | THR | B | 2 | −14.898 | 18.510 | 42.180 | 1.00 | 199.64 | B |
| ATOM | 3622 | CA | THR | B | 2 | −15.383 | 17.161 | 41.889 | 1.00 | 199.64 | B |
| ATOM | 3623 | CB | THR | B | 2 | −16.639 | 17.199 | 40.977 | 1.00 | 121.84 | B |
| ATOM | 3624 | OG1 | THR | B | 2 | −17.657 | 18.006 | 41.582 | 1.00 | 120.58 | B |
| ATOM | 3625 | CG2 | THR | B | 2 | −17.179 | 15.786 | 40.747 | 1.00 | 123.73 | B |
| ATOM | 3626 | C | THR | B | 2 | −14.327 | 16.327 | 41.170 | 1.00 | 199.64 | B |
| ATOM | 3627 | O | THR | B | 2 | −14.014 | 16.604 | 40.011 | 1.00 | 199.64 | B |
| ATOM | 3628 | N | PRO | B | 3 | −13.750 | 15.306 | 41.841 | 1.00 | 200.84 | B |
| ATOM | 3629 | CD | PRO | B | 3 | −13.820 | 14.857 | 43.246 | 1.00 | 80.10 | B |
| ATOM | 3630 | CA | PRO | B | 3 | −12.753 | 14.533 | 41.096 | 1.00 | 200.84 | B |
| ATOM | 3631 | CB | PRO | B | 3 | −12.528 | 13.310 | 41.989 | 1.00 | 78.34 | B |
| ATOM | 3632 | CG | PRO | B | 3 | −12.606 | 13.914 | 43.362 | 1.00 | 78.21 | B |
| ATOM | 3633 | C | PRO | B | 3 | −13.337 | 14.197 | 39.720 | 1.00 | 200.84 | B |
| ATOM | 3634 | O | PRO | B | 3 | −12.612 | 14.165 | 38.723 | 1.00 | 200.84 | B |
| ATOM | 3635 | N | SER | B | 4 | −14.655 | 13.977 | 39.682 | 1.00 | 135.14 | B |
| ATOM | 3636 | CA | SER | B | 4 | −15.386 | 13.689 | 38.444 | 1.00 | 131.71 | B |
| ATOM | 3637 | CB | SER | B | 4 | −14.551 | 12.832 | 37.483 | 1.00 | 158.37 | B |
| ATOM | 3638 | OG | SER | B | 4 | −14.634 | 11.457 | 37.815 | 1.00 | 160.32 | B |
| ATOM | 3639 | C | SER | B | 4 | −16.727 | 12.994 | 38.649 | 1.00 | 132.05 | B |
| ATOM | 3640 | O | SER | B | 4 | −17.650 | 13.211 | 37.865 | 1.00 | 132.20 | B |
| ATOM | 3641 | N | ASP | B | 5 | −16.822 | 12.169 | 39.695 | 1.00 | 59.17 | B |
| ATOM | 3642 | CA | ASP | B | 5 | −18.032 | 11.390 | 40.012 | 1.00 | 59.28 | B |
| ATOM | 3643 | CB | ASP | B | 5 | −19.232 | 11.931 | 39.216 | 1.00 | 140.25 | B |
| ATOM | 3644 | CG | ASP | B | 5 | −20.499 | 11.140 | 39.439 | 1.00 | 140.25 | B |
| ATOM | 3645 | OD1 | ASP | B | 5 | −21.484 | 11.386 | 38.707 | 1.00 | 140.25 | B |
| ATOM | 3646 | OD2 | ASP | B | 5 | −20.519 | 10.280 | 40.342 | 1.00 | 138.81 | B |
| ATOM | 3647 | C | ASP | B | 5 | −17.782 | 9.904 | 39.663 | 1.00 | 56.78 | B |
| ATOM | 3648 | O | ASP | B | 5 | −18.417 | 9.005 | 40.206 | 1.00 | 60.29 | B |
| ATOM | 3649 | N | LYS | B | 6 | −16.833 | 9.662 | 38.762 | 1.00 | 55.52 | B |
| ATOM | 3650 | CA | LYS | B | 6 | −16.483 | 8.314 | 38.312 | 1.00 | 51.90 | B |
| ATOM | 3651 | CB | LYS | B | 6 | −15.607 | 8.372 | 37.053 | 1.00 | 48.08 | B |
| ATOM | 3652 | CG | LYS | B | 6 | −16.228 | 8.822 | 35.760 | 1.00 | 54.49 | B |
| ATOM | 3653 | CD | LYS | B | 6 | −15.123 | 8.942 | 34.711 | 1.00 | 58.10 | B |
| ATOM | 3654 | CE | LYS | B | 6 | −15.679 | 9.163 | 33.325 | 1.00 | 61.37 | B |
| ATOM | 3655 | NZ | LYS | B | 6 | −16.498 | 7.986 | 32.898 | 1.00 | 62.56 | B |
| ATOM | 3656 | C | LYS | B | 6 | −15.663 | 7.554 | 39.331 | 1.00 | 47.23 | B |
| ATOM | 3657 | O | LYS | B | 6 | −14.705 | 8.102 | 39.873 | 1.00 | 51.11 | B |
| ATOM | 3658 | N | PRO | B | 7 | −16.015 | 6.290 | 39.613 | 1.00 | 37.62 | B |
| ATOM | 3659 | CD | PRO | B | 7 | −17.210 | 5.510 | 39.293 | 1.00 | 29.09 | B |
| ATOM | 3660 | CA | PRO | B | 7 | −15.188 | 5.567 | 40.580 | 1.00 | 35.66 | B |
| ATOM | 3661 | CB | PRO | B | 7 | −15.873 | 4.203 | 40.694 | 1.00 | 31.09 | B |
| ATOM | 3662 | CG | PRO | B | 7 | −16.687 | 4.102 | 39.479 | 1.00 | 29.96 | B |
| ATOM | 3663 | C | PRO | B | 7 | −13.793 | 5.473 | 39.964 | 1.00 | 33.72 | B |
| ATOM | 3664 | O | PRO | B | 7 | −13.652 | 5.165 | 38.774 | 1.00 | 34.73 | B |
| ATOM | 3665 | N | VAL | B | 8 | −12.771 | 5.768 | 40.765 | 1.00 | 26.72 | B |
| ATOM | 3666 | CA | VAL | B | 8 | −11.402 | 5.748 | 40.280 | 1.00 | 27.88 | B |
| ATOM | 3667 | CB | VAL | B | 8 | −11.050 | 7.107 | 39.625 | 1.00 | 25.59 | B |
| ATOM | 3668 | CG1 | VAL | B | 8 | −11.258 | 8.232 | 40.622 | 1.00 | 26.17 | B |
| ATOM | 3669 | CG2 | VAL | B | 8 | −9.614 | 7.108 | 39.126 | 1.00 | 28.29 | B |
| ATOM | 3670 | C | VAL | B | 8 | −10.424 | 5.478 | 41.416 | 1.00 | 30.38 | B |
| ATOM | 3671 | O | VAL | B | 8 | −10.738 | 5.660 | 42.598 | 1.00 | 29.62 | B |
| ATOM | 3672 | N | ALA | B | 9 | −9.227 | 5.043 | 41.059 | 1.00 | 29.74 | B |
| ATOM | 3673 | CA | ALA | B | 9 | −8.221 | 4.799 | 42.062 | 1.00 | 35.98 | B |
| ATOM | 3674 | CB | ALA | B | 9 | −8.564 | 3.573 | 42.847 | 1.00 | 29.93 | B |
| ATOM | 3675 | C | ALA | B | 9 | −6.866 | 4.627 | 41.420 | 1.00 | 36.96 | B |
| ATOM | 3676 | O | ALA | B | 9 | −6.732 | 3.983 | 40.377 | 1.00 | 34.94 | B |
| ATOM | 3677 | N | HIS | B | 10 | −5.866 | 5.238 | 42.035 | 1.00 | 33.63 | B |
| ATOM | 3678 | CA | HIS | B | 10 | −4.497 | 5.117 | 41.580 | 1.00 | 34.50 | B |
| ATOM | 3679 | CB | HIS | B | 10 | −4.050 | 6.361 | 40.817 | 1.00 | 30.60 | B |
| ATOM | 3680 | CG | HIS | B | 10 | −2.651 | 6.266 | 40.293 | 1.00 | 32.09 | B |
| ATOM | 3681 | CD2 | HIS | B | 10 | −1.481 | 6.727 | 40.793 | 1.00 | 29.82 | B |
| ATOM | 3682 | ND1 | HIS | B | 10 | −2.328 | 5.564 | 39.153 | 1.00 | 32.23 | B |
| ATOM | 3683 | CE1 | HIS | B | 10 | −1.021 | 5.592 | 38.973 | 1.00 | 32.58 | B |
| ATOM | 3684 | NE2 | HIS | B | 10 | −0.484 | 6.291 | 39.956 | 1.00 | 30.06 | B |
| ATOM | 3685 | C | HIS | B | 10 | −3.709 | 4.991 | 42.872 | 1.00 | 32.71 | B |
| ATOM | 3686 | O | HIS | B | 10 | −3.425 | 5.994 | 43.543 | 1.00 | 30.87 | B |
| ATOM | 3687 | N | VAL | B | 11 | −3.397 | 3.760 | 43.250 | 1.00 | 39.61 | B |
| ATOM | 3688 | CA | VAL | B | 11 | −2.652 | 3.531 | 44.478 | 1.00 | 42.19 | B |
| ATOM | 3689 | CB | VAL | B | 11 | −3.237 | 2.356 | 45.285 | 1.00 | 32.32 | B |
| ATOM | 3690 | CG1 | VAL | B | 11 | −4.664 | 2.693 | 45.669 | 1.00 | 28.38 | B |
| ATOM | 3691 | CG2 | VAL | B | 11 | −3.204 | 1.062 | 44.474 | 1.00 | 34.30 | B |
| ATOM | 3692 | C | VAL | B | 11 | −1.205 | 3.275 | 44.149 | 1.00 | 40.80 | B |
| ATOM | 3693 | O | VAL | B | 11 | −0.872 | 2.824 | 43.055 | 1.00 | 37.81 | B |
| ATOM | 3694 | N | VAL | B | 12 | −0.339 | 3.568 | 45.100 | 1.00 | 44.54 | B |
| ATOM | 3695 | CA | VAL | B | 12 | 1.078 | 3.408 | 44.874 | 1.00 | 42.71 | B |
| ATOM | 3696 | CB | VAL | B | 12 | 1.718 | 4.790 | 44.850 | 1.00 | 46.95 | B |
| ATOM | 3697 | CG1 | VAL | B | 12 | 1.764 | 5.358 | 46.258 | 1.00 | 40.75 | B |
| ATOM | 3698 | CG2 | VAL | B | 12 | 3.084 | 4.717 | 44.221 | 1.00 | 50.42 | B |
| ATOM | 3699 | C | VAL | B | 12 | 1.692 | 2.532 | 45.962 | 1.00 | 40.67 | B |
| ATOM | 3700 | O | VAL | B | 12 | 1.097 | 2.365 | 47.034 | 1.00 | 40.51 | B |
| ATOM | 3701 | N | ALA | B | 13 | 2.873 | 1.979 | 45.693 | 1.00 | 56.19 | B |
| ATOM | 3702 | CA | ALA | B | 13 | 3.550 | 1.097 | 46.656 | 1.00 | 55.86 | B |
| ATOM | 3703 | CB | ALA | B | 13 | 4.666 | 0.319 | 45.971 | 1.00 | 19.80 | B |
| ATOM | 3704 | C | ALA | B | 13 | 4.119 | 1.812 | 47.862 | 1.00 | 59.84 | B |
| ATOM | 3705 | O | ALA | B | 13 | 4.676 | 2.897 | 47.736 | 1.00 | 57.35 | B |
| ATOM | 3706 | N | ASN | B | 14 | 3.988 | 1.185 | 49.028 | 1.00 | 49.54 | B |
| ATOM | 3707 | CA | ASN | B | 14 | 4.494 | 1.737 | 50.284 | 1.00 | 50.66 | B |
| ATOM | 3708 | CB | ASN | B | 14 | 3.733 | 1.132 | 51.455 | 1.00 | 57.90 | B |
| ATOM | 3709 | CG | ASN | B | 14 | 4.210 | 1.653 | 52.793 | 1.00 | 59.89 | B |
| ATOM | 3710 | OD1 | ASN | B | 14 | 3.745 | 1.205 | 53.843 | 1.00 | 59.47 | B |
| ATOM | 3711 | ND2 | ASN | B | 14 | 5.140 | 2.606 | 52.766 | 1.00 | 62.01 | B |
| ATOM | 3712 | C | ASN | B | 14 | 5.974 | 1.399 | 50.412 | 1.00 | 53.54 | B |
| ATOM | 3713 | O | ASN | B | 14 | 6.335 | 0.226 | 50.504 | 1.00 | 54.06 | B |
| ATOM | 3714 | N | PRO | B | 15 | 6.852 | 2.419 | 50.434 | 1.00 | 62.22 | B |
| ATOM | 3715 | CD | PRO | B | 15 | 6.601 | 3.863 | 50.316 | 1.00 | 38.30 | B |
| ATOM | 3716 | CA | PRO | B | 15 | 8.291 | 2.150 | 50.543 | 1.00 | 65.82 | B |
| ATOM | 3717 | CB | PRO | B | 15 | 8.936 | 3.470 | 50.098 | 1.00 | 42.64 | B |
| ATOM | 3718 | CG | PRO | B | 15 | 7.794 | 4.292 | 49.515 | 1.00 | 40.91 | B |
| ATOM | 3719 | C | PRO | B | 15 | 8.709 | 1.757 | 51.956 | 1.00 | 67.03 | B |
| ATOM | 3720 | O | PRO | B | 15 | 9.766 | 1.158 | 52.160 | 1.00 | 69.06 | B |
| ATOM | 3721 | N | GLN | B | 16 | 7.866 | 2.095 | 52.925 | 1.00 | 89.15 | B |
| ATOM | 3722 | CA | GLN | B | 16 | 8.141 | 1.802 | 54.327 | 1.00 | 88.10 | B |
| ATOM | 3723 | CB | GLN | B | 16 | 7.324 | 2.750 | 55.214 | 1.00 | 104.52 | B |
| ATOM | 3724 | CG | GLN | B | 16 | 7.832 | 2.914 | 56.641 | 1.00 | 107.45 | B |
| ATOM | 3725 | CD | GLN | B | 16 | 9.253 | 3.451 | 56.703 | 1.00 | 109.24 | B |
| ATOM | 3726 | OE1 | GLN | B | 16 | 9.610 | 4.380 | 55.975 | 1.00 | 111.36 | B |
| ATOM | 3727 | NE2 | GLN | B | 16 | 10.068 | 2.875 | 57.584 | 1.00 | 107.89 | B |
| ATOM | 3728 | C | GLN | B | 16 | 7.829 | 0.345 | 54.682 | 1.00 | 84.77 | B |
| ATOM | 3729 | O | GLN | B | 16 | 7.958 | −0.060 | 55.840 | 1.00 | 85.34 | B |
| ATOM | 3730 | N | ALA | B | 17 | 7.422 | −0.444 | 53.688 | 1.00 | 76.70 | B |
| ATOM | 3731 | CA | ALA | B | 17 | 7.104 | −1.852 | 53.923 | 1.00 | 80.46 | B |
| ATOM | 3732 | CB | ALA | B | 17 | 5.651 | −2.131 | 53.568 | 1.00 | 40.98 | B |
| ATOM | 3733 | C | ALA | B | 17 | 8.024 | −2.759 | 53.117 | 1.00 | 84.96 | B |
| ATOM | 3734 | O | ALA | B | 17 | 7.580 | −3.497 | 52.243 | 1.00 | 86.60 | B |
| ATOM | 3735 | N | GLU | B | 18 | 9.314 | −2.699 | 53.429 | 1.00 | 94.79 | B |
| ATOM | 3736 | CA | GLU | B | 18 | 10.318 | −3.501 | 52.744 | 1.00 | 96.79 | B |
| ATOM | 3737 | CB | GLU | B | 18 | 11.662 | −3.387 | 53.466 | 1.00 | 98.21 | B |
| ATOM | 3738 | CG | GLU | B | 18 | 11.630 | −3.885 | 54.902 | 1.00 | 98.21 | B |
| ATOM | 3739 | CD | GLU | B | 18 | 13.000 | −3.885 | 55.545 | 1.00 | 98.21 | B |
| ATOM | 3740 | OE1 | GLU | B | 18 | 13.893 | −4.591 | 55.028 | 1.00 | 98.21 | B |
| ATOM | 3741 | OE2 | GLU | B | 18 | 13.187 | −3.183 | 56.562 | 1.00 | 98.21 | B |
| ATOM | 3742 | C | GLU | B | 18 | 9.930 | −4.971 | 52.648 | 1.00 | 96.45 | B |
| ATOM | 3743 | O | GLU | B | 18 | 9.246 | −5.509 | 53.523 | 1.00 | 96.06 | B |
| ATOM | 3744 | N | GLY | B | 19 | 10.373 | −5.606 | 51.569 | 1.00 | 77.61 | B |
| ATOM | 3745 | CA | GLY | B | 19 | 10.099 | −7.015 | 51.366 | 1.00 | 75.10 | B |
| ATOM | 3746 | C | GLY | B | 19 | 8.719 | −7.384 | 50.862 | 1.00 | 73.60 | B |
| ATOM | 3747 | O | GLY | B | 19 | 8.480 | −8.542 | 50.517 | 1.00 | 73.61 | B |
| ATOM | 3748 | N | GLN | B | 20 | 7.804 | −6.423 | 50.807 | 1.00 | 101.28 | B |
| ATOM | 3749 | CA | GLN | B | 20 | 6.456 | −6.714 | 50.336 | 1.00 | 96.85 | B |
| ATOM | 3750 | CB | GLN | B | 20 | 5.543 | −6.970 | 51.530 | 1.00 | 103.39 | B |
| ATOM | 3751 | CG | GLN | B | 20 | 5.577 | −5.857 | 52.551 | 1.00 | 104.37 | B |
| ATOM | 3752 | CD | GLN | B | 20 | 4.728 | −6.157 | 53.762 | 1.00 | 104.13 | B |
| ATOM | 3753 | OE1 | GLN | B | 20 | 4.952 | −7.148 | 54.456 | 1.00 | 103.78 | B |
| ATOM | 3754 | NE2 | GLN | B | 20 | 3.743 | −5.301 | 54.025 | 1.00 | 103.58 | B |
| ATOM | 3755 | C | GLN | B | 20 | 5.878 | −5.600 | 49.468 | 1.00 | 91.59 | B |
| ATOM | 3756 | O | GLN | B | 20 | 6.448 | −4.514 | 49.364 | 1.00 | 90.30 | B |
| ATOM | 3757 | N | LEU | B | 21 | 4.742 | −5.879 | 48.839 | 1.00 | 60.98 | B |
| ATOM | 3758 | CA | LEU | B | 21 | 4.090 | −4.902 | 47.981 | 1.00 | 57.44 | B |
| ATOM | 3759 | CB | LEU | B | 21 | 3.877 | −5.482 | 46.582 | 1.00 | 47.74 | B |
| ATOM | 3760 | CG | LEU | B | 21 | 3.138 | −4.621 | 45.550 | 1.00 | 46.52 | B |
| ATOM | 3761 | CD1 | LEU | B | 21 | 3.722 | −3.215 | 45.520 | 1.00 | 43.82 | B |
| ATOM | 3762 | CD2 | LEU | B | 21 | 3.247 | −5.282 | 44.180 | 1.00 | 48.56 | B |
| ATOM | 3763 | C | LEU | B | 21 | 2.755 | −4.501 | 48.575 | 1.00 | 56.05 | B |
| ATOM | 3764 | O | LEU | B | 21 | 1.742 | −5.149 | 48.339 | 1.00 | 51.37 | B |
| ATOM | 3765 | N | GLN | B | 22 | 2.759 | −3.424 | 49.349 | 1.00 | 59.30 | B |
| ATOM | 3766 | CA | GLN | B | 22 | 1.545 | −2.935 | 49.993 | 1.00 | 63.90 | B |
| ATOM | 3767 | CB | GLN | B | 22 | 1.834 | −2.728 | 51.480 | 1.00 | 55.04 | B |
| ATOM | 3768 | CG | GLN | B | 22 | 0.737 | −2.058 | 52.266 | 1.00 | 61.81 | B |
| ATOM | 3769 | CD | GLN | B | 22 | 1.212 | −1.604 | 53.637 | 1.00 | 68.03 | B |
| ATOM | 3770 | OE1 | GLN | B | 22 | 0.489 | −0.927 | 54.371 | 1.00 | 73.26 | B |
| ATOM | 3771 | NE2 | GLN | B | 22 | 2.438 | −1.977 | 53.986 | 1.00 | 68.79 | B |
| ATOM | 3772 | C | GLN | B | 22 | 1.104 | −1.627 | 49.331 | 1.00 | 61.29 | B |
| ATOM | 3773 | O | GLN | B | 22 | 1.878 | −0.691 | 49.235 | 1.00 | 60.24 | B |
| ATOM | 3774 | N | TRP | B | 23 | −0.141 | −1.569 | 48.876 | 1.00 | 40.96 | B |
| ATOM | 3775 | CA | TRP | B | 23 | −0.669 | −0.377 | 48.205 | 1.00 | 34.84 | B |
| ATOM | 3776 | CB | TRP | B | 23 | −1.766 | −0.761 | 47.207 | 1.00 | 32.27 | B |
| ATOM | 3777 | CG | TRP | B | 23 | −1.375 | −1.775 | 46.196 | 1.00 | 32.79 | B |
| ATOM | 3778 | CD2 | TRP | B | 23 | −0.444 | −1.606 | 45.114 | 1.00 | 30.90 | B |
| ATOM | 3779 | CE2 | TRP | B | 23 | −0.384 | −2.836 | 44.420 | 1.00 | 32.52 | B |
| ATOM | 3780 | CE3 | TRP | B | 23 | 0.347 | −0.537 | 44.666 | 1.00 | 30.59 | B |
| ATOM | 3781 | CD1 | TRP | B | 23 | −1.828 | −3.049 | 46.116 | 1.00 | 32.26 | B |
| ATOM | 3782 | NE1 | TRP | B | 23 | −1.240 | −3.696 | 45.051 | 1.00 | 31.35 | B |
| ATOM | 3783 | CZ2 | TRP | B | 23 | 0.436 | −3.030 | 43.301 | 1.00 | 34.58 | B |
| ATOM | 3784 | CZ3 | TRP | B | 23 | 1.160 | −0.730 | 43.553 | 1.00 | 35.03 | B |
| ATOM | 3785 | CH2 | TRP | B | 23 | 1.196 | −1.970 | 42.886 | 1.00 | 32.84 | B |
| ATOM | 3786 | C | TRP | B | 23 | −1.262 | 0.663 | 49.138 | 1.00 | 35.34 | B |
| ATOM | 3787 | O | TRP | B | 23 | −1.986 | 0.323 | 50.074 | 1.00 | 36.62 | B |
| ATOM | 3788 | N | LEU | B | 24 | −0.962 | 1.933 | 48.881 | 1.00 | 39.24 | B |
| ATOM | 3789 | CA | LEU | B | 24 | −1.529 | 3.013 | 49.692 | 1.00 | 36.93 | B |
| ATOM | 3790 | CB | LEU | B | 24 | −0.507 | 3.685 | 50.625 | 1.00 | 48.14 | B |
| ATOM | 3791 | CG | LEU | B | 24 | 0.827 | 3.044 | 50.956 | 1.00 | 48.87 | B |
| ATOM | 3792 | CD1 | LEU | B | 24 | 1.704 | 3.142 | 49.726 | 1.00 | 50.49 | B |
| ATOM | 3793 | CD2 | LEU | B | 24 | 1.476 | 3.762 | 52.110 | 1.00 | 43.98 | B |
| ATOM | 3794 | C | LEU | B | 24 | −2.098 | 4.075 | 48.783 | 1.00 | 43.79 | B |
| ATOM | 3795 | O | LEU | B | 24 | −1.725 | 4.185 | 47.613 | 1.00 | 40.77 | B |
| ATOM | 3796 | N | ASN | B | 25 | −3.005 | 4.860 | 49.342 | 1.00 | 34.94 | B |
| ATOM | 3797 | CA | ASN | B | 25 | −3.642 | 5.929 | 48.605 | 1.00 | 36.38 | B |
| ATOM | 3798 | CB | ASN | B | 25 | −5.134 | 5.668 | 48.514 | 1.00 | 43.12 | B |
| ATOM | 3799 | CG | ASN | B | 25 | −5.823 | 5.907 | 49.827 | 1.00 | 44.69 | B |
| ATOM | 3600 | OD1 | ASN | B | 25 | −6.623 | 6.829 | 49.958 | 1.00 | 48.68 | B |
| ATOM | 3801 | ND2 | ASN | B | 25 | −5.499 | 5.088 | 50.823 | 1.00 | 41.09 | B |
| ATOM | 3802 | C | ASN | B | 25 | −3.419 | 7.257 | 49.321 | 1.00 | 36.91 | B |
| ATOM | 3803 | O | ASN | B | 25 | −3.834 | 8.302 | 48.835 | 1.00 | 39.27 | B |
| ATOM | 3804 | N | ARG | B | 26 | −2.769 | 7.214 | 50.476 | 1.00 | 42.06 | B |
| ATOM | 3805 | CA | ARG | B | 26 | −2.520 | 8.428 | 51.247 | 1.00 | 43.34 | B |
| ATOM | 3806 | CB | ARG | B | 26 | −2.493 | 8.092 | 52.744 | 1.00 | 48.75 | B |
| ATOM | 3807 | CG | ARG | B | 26 | −1.480 | 7.026 | 53.138 | 1.00 | 49.66 | B |
| ATOM | 3808 | CD | ARG | B | 26 | −1.310 | 6.985 | 54.646 | 1.00 | 53.72 | B |
| ATOM | 3809 | NE | ARG | B | 26 | −0.358 | 5.961 | 55.060 | 1.00 | 54.86 | B |
| ATOM | 3810 | CZ | ARG | B | 26 | −0.661 | 4.678 | 55.206 | 1.00 | 55.42 | B |
| ATOM | 3811 | NH1 | ARG | B | 26 | −1.896 | 4.257 | 54.976 | 1.00 | 55.90 | B |
| ATOM | 3812 | NH2 | ARG | B | 26 | 0.269 | 3.817 | 55.582 | 1.00 | 54.45 | B |
| ATOM | 3813 | C | ARG | B | 26 | −1.230 | 9.176 | 50.870 | 1.00 | 41.40 | B |
| ATOM | 3814 | O | ARG | B | 26 | −0.459 | 9.584 | 51.740 | 1.00 | 41.11 | B |
| ATOM | 3815 | N | ARG | B | 27 | −1.000 | 9.365 | 49.575 | 1.00 | 40.77 | B |
| ATOM | 3816 | CA | ARG | B | 27 | 0.196 | 10.052 | 49.126 | 1.00 | 41.70 | B |
| ATOM | 3817 | CB | ARG | B | 27 | 1.206 | 9.051 | 48.577 | 1.00 | 35.01 | B |
| ATOM | 3818 | CG | ARG | B | 27 | 1.770 | 8.148 | 49.631 | 1.00 | 36.43 | B |
| ATOM | 3819 | CD | ARG | B | 27 | 2.301 | 8.960 | 50.798 | 1.00 | 38.80 | B |
| ATOM | 3820 | NE | ARG | B | 27 | 2.756 | 8.138 | 51.911 | 1.00 | 42.19 | B |
| ATOM | 3821 | CZ | ARG | B | 27 | 3.764 | 7.276 | 51.834 | 1.00 | 45.80 | B |
| ATOM | 3822 | NH1 | ARG | B | 27 | 4.101 | 6.574 | 52.910 | 1.00 | 46.67 | B |
| ATOM | 3823 | NH2 | ARG | B | 27 | 4.433 | 7.116 | 50.685 | 1.00 | 44.03 | B |
| ATOM | 3824 | C | ARG | B | 27 | −0.050 | 11.136 | 48.097 | 1.00 | 42.16 | B |
| ATOM | 3825 | O | ARG | B | 27 | −1.170 | 11.337 | 47.625 | 1.00 | 42.30 | B |
| ATOM | 3826 | N | ALA | B | 28 | 1.036 | 11.820 | 47.766 | 1.00 | 76.46 | B |
| ATOM | 3827 | CA | ALA | B | 28 | 1.061 | 12.915 | 46.816 | 1.00 | 75.36 | B |
| ATOM | 3828 | CB | ALA | B | 28 | 2.443 | 13.012 | 46.197 | 1.00 | 125.57 | B |
| ATOM | 3829 | C | ALA | B | 28 | 0.021 | 12.873 | 45.720 | 1.00 | 75.02 | B |
| ATOM | 3830 | O | ALA | B | 28 | −1.050 | 13.461 | 45.845 | 1.00 | 76.04 | B |
| ATOM | 3831 | N | ASN | B | 29 | 0.328 | 12.185 | 44.633 | 1.00 | 56.89 | B |
| ATOM | 3832 | CA | ASN | B | 29 | −0.614 | 12.144 | 43.521 | 1.00 | 56.06 | B |
| ATOM | 3833 | CB | ASN | B | 29 | 0.113 | 12.535 | 42.234 | 1.00 | 40.41 | B |
| ATOM | 3834 | CG | ASN | B | 29 | 0.643 | 13.958 | 42.281 | 1.00 | 40.47 | B |
| ATOM | 3835 | OD1 | ASN | B | 29 | −0.091 | 14.913 | 42.035 | 1.00 | 46.33 | B |
| ATOM | 3836 | ND2 | ASN | B | 29 | 1.920 | 14.105 | 42.625 | 1.00 | 37.48 | B |
| ATOM | 3837 | C | ASN | B | 29 | −1.284 | 10.793 | 43.364 | 1.00 | 54.40 | B |
| ATOM | 3838 | O | ASN | B | 29 | −1.163 | 10.148 | 42.324 | 1.00 | 52.12 | B |
| ATOM | 3839 | N | ALA | B | 30 | −1.993 | 10.369 | 44.405 | 1.00 | 46.66 | B |
| ATOM | 3840 | CA | ALA | B | 30 | −2.683 | 9.089 | 44.393 | 1.00 | 41.48 | B |
| ATOM | 3841 | CB | ALA | B | 30 | −2.212 | 8.249 | 45.552 | 1.00 | 33.13 | B |
| ATOM | 3842 | C | ALA | B | 30 | −4.190 | 9.321 | 44.475 | 1.00 | 42.88 | B |
| ATOM | 3843 | O | ALA | B | 30 | −4.642 | 10.440 | 44.746 | 1.00 | 47.03 | B |
| ATOM | 3844 | N | LEU | B | 31 | −4.966 | 8.270 | 44.238 | 1.00 | 42.00 | B |
| ATOM | 3845 | CA | LEU | B | 31 | −6.416 | 8.395 | 44.269 | 1.00 | 41.47 | B |
| ATOM | 3846 | CB | LEU | B | 31 | −6.952 | 8.668 | 42.873 | 1.00 | 34.85 | B |
| ATOM | 3847 | CG | LEU | B | 31 | −6.745 | 10.003 | 42.201 | 1.00 | 37.73 | B |
| ATOM | 3848 | CD1 | LEU | B | 31 | −7.047 | 9.870 | 40.716 | 1.00 | 35.35 | B |
| ATOM | 3849 | CD2 | LEU | B | 31 | −7.645 | 11.021 | 42.853 | 1.00 | 37.20 | B |
| ATOM | 3850 | C | LEU | B | 31 | −7.128 | 7.153 | 44.784 | 1.00 | 40.73 | B |
| ATOM | 3851 | O | LEU | B | 31 | −6.665 | 6.023 | 44.604 | 1.00 | 41.54 | B |
| ATOM | 3852 | N | LEU | B | 32 | −8.277 | 7.387 | 45.409 | 1.00 | 39.38 | B |
| ATOM | 3853 | CA | LEU | B | 32 | −9.143 | 6.334 | 45.931 | 1.00 | 39.17 | B |
| ATOM | 3854 | CB | LEU | B | 32 | −8.588 | 5.758 | 47.228 | 1.00 | 25.67 | B |
| ATOM | 3855 | CG | LEU | B | 32 | −9.438 | 4.575 | 47.717 | 1.00 | 24.25 | B |
| ATOM | 3856 | CD1 | LEU | B | 32 | −9.355 | 3.402 | 46.721 | 1.00 | 23.23 | B |
| ATOM | 3857 | CD2 | LEU | B | 32 | −8.963 | 4.157 | 49.110 | 1.00 | 24.57 | B |
| ATOM | 3858 | C | LEU | B | 32 | −10.472 | 7.030 | 46.194 | 1.00 | 38.81 | B |
| ATOM | 3859 | O | LEU | B | 32 | −10.362 | 7.428 | 47.318 | 1.00 | 44.68 | B |
| ATOM | 3860 | N | ALA | B | 33 | −11.280 | 7.173 | 45.150 | 1.00 | 39.73 | B |
| ATOM | 3861 | CA | ALA | B | 33 | −12.534 | 7.888 | 45.291 | 1.00 | 40.25 | B |
| ATOM | 3862 | CB | ALA | B | 33 | −12.350 | 9.313 | 44.808 | 1.00 | 40.67 | B |
| ATOM | 3863 | C | ALA | B | 33 | −13.764 | 7.311 | 44.629 | 1.00 | 42.04 | B |
| ATOM | 3864 | O | ALA | B | 33 | −13.711 | 6.344 | 43.873 | 1.00 | 46.22 | B |
| ATOM | 3865 | N | ASN | B | 34 | −14.879 | 7.965 | 44.931 | 1.00 | 42.37 | B |
| ATOM | 3866 | CA | ASN | B | 34 | −16.184 | 7.625 | 44.404 | 1.00 | 39.56 | B |
| ATOM | 3867 | CB | ASN | B | 34 | −16.256 | 8.047 | 42.951 | 1.00 | 42.30 | B |
| ATOM | 3868 | CG | ASN | B | 34 | −16.033 | 9.514 | 42.788 | 1.00 | 46.22 | B |
| ATOM | 3869 | OD1 | ASN | B | 34 | −16.670 | 10.316 | 43.464 | 1.00 | 48.70 | B |
| ATOM | 3870 | ND2 | ASN | B | 34 | −15.126 | 9.886 | 41.899 | 1.00 | 41.63 | B |
| ATOM | 3871 | C | ASN | B | 34 | −16.621 | 6.187 | 44.548 | 1.00 | 40.31 | B |
| ATOM | 3872 | O | ASN | B | 34 | −17.198 | 5.620 | 43.635 | 1.00 | 42.85 | B |
| ATOM | 3873 | N | GLY | B | 35 | −16.345 | 5.591 | 45.693 | 1.00 | 42.99 | B |
| ATOM | 3874 | CA | GLY | B | 35 | −16.794 | 4.241 | 45.892 | 1.00 | 37.58 | B |
| ATOM | 3875 | C | GLY | B | 35 | −15.779 | 3.130 | 45.911 | 1.00 | 37.93 | B |
| ATOM | 3876 | O | GLY | B | 35 | −15.967 | 2.168 | 46.652 | 1.00 | 39.42 | B |
| ATOM | 3877 | N | VAL | B | 36 | −14.718 | 3.202 | 45.120 | 1.00 | 35.94 | B |
| ATOM | 3878 | CA | VAL | B | 36 | −13.791 | 2.084 | 45.167 | 1.00 | 37.27 | B |
| ATOM | 3879 | CB | VAL | B | 36 | −12.804 | 2.088 | 43.950 | 1.00 | 37.76 | B |
| ATOM | 3880 | CG1 | VAL | B | 36 | −12.938 | 3.368 | 43.193 | 1.00 | 35.79 | B |
| ATOM | 3881 | CG2 | VAL | B | 36 | −11.379 | 1.836 | 44.395 | 1.00 | 38.16 | B |
| ATOM | 3882 | C | VAL | B | 36 | −13.085 | 2.062 | 46.513 | 1.00 | 41.42 | B |
| ATOM | 3883 | O | VAL | B | 36 | −12.752 | 3.101 | 47.076 | 1.00 | 42.38 | B |
| ATOM | 3884 | N | GLU | B | 37 | −12.909 | 0.862 | 47.048 | 1.00 | 42.35 | B |
| ATOM | 3885 | CA | GLU | B | 37 | −12.272 | 0.689 | 48.340 | 1.00 | 44.95 | B |
| ATOM | 3886 | CB | GLU | B | 37 | −13.225 | −0.028 | 49.292 | 1.00 | 78.55 | B |
| ATOM | 3887 | CG | GLU | B | 37 | −14.669 | 0.388 | 49.105 | 1.00 | 85.58 | B |
| ATOM | 3888 | CD | GLU | B | 37 | −15.663 | −0.555 | 49.767 | 1.00 | 88.55 | B |
| ATOM | 3889 | OE1 | GLU | B | 37 | −15.367 | −1.768 | 49.875 | 1.00 | 91.98 | B |
| ATOM | 3890 | OE2 | GLU | B | 37 | −16.754 | −0.084 | 50.159 | 1.00 | 89.11 | B |
| ATOM | 3891 | C | GLU | B | 37 | −10.992 | −0.124 | 48.201 | 1.00 | 43.84 | B |
| ATOM | 3892 | O | GLU | B | 37 | −10.827 | −0.920 | 47.270 | 1.00 | 46.30 | B |
| ATOM | 3893 | N | LEU | B | 38 | −10.075 | 0.111 | 49.126 | 1.00 | 51.43 | B |
| ATOM | 3894 | CA | LEU | B | 38 | −8.821 | −0.609 | 49.153 | 1.00 | 53.47 | B |
| ATOM | 3895 | CB | LEU | B | 38 | −7.654 | 0.372 | 49.296 | 1.00 | 29.47 | B |
| ATOM | 3896 | CG | LEU | B | 38 | −6.257 | −0.246 | 49.265 | 1.00 | 27.38 | B |
| ATOM | 3897 | CD1 | LEU | B | 38 | −6.132 | −1.195 | 48.088 | 1.00 | 25.76 | B |
| ATOM | 3898 | CD2 | LEU | B | 38 | −5.218 | 0.867 | 49.201 | 1.00 | 24.25 | B |
| ATOM | 3899 | C | LEU | B | 38 | −8.979 | −1.489 | 50.386 | 1.00 | 53.74 | B |
| ATOM | 3900 | O | LEU | B | 38 | −8.874 | −1.026 | 51.526 | 1.00 | 47.35 | B |
| ATOM | 3901 | N | ARG | B | 39 | −9.262 | −2.762 | 50.141 | 1.00 | 41.67 | B |
| ATOM | 3902 | CA | ARG | B | 39 | −9.502 | −3.729 | 51.207 | 1.00 | 48.20 | B |
| ATOM | 3903 | CB | ARG | B | 39 | −10.963 | −4.167 | 51.128 | 1.00 | 65.95 | B |
| ATOM | 3904 | CG | ARG | B | 39 | −11.461 | −4.949 | 52.291 | 1.00 | 73.65 | B |
| ATOM | 3905 | CD | ARG | B | 39 | −12.967 | −5.058 | 52.210 | 1.00 | 82.26 | B |
| ATOM | 3906 | NE | ARG | B | 39 | −13.526 | −5.567 | 53.461 | 1.00 | 88.79 | B |
| ATOM | 3907 | CZ | ARG | B | 39 | −14.822 | −5.543 | 53.772 | 1.00 | 89.59 | B |
| ATOM | 3908 | NH1 | ARG | B | 39 | −15.698 | −5.035 | 52.914 | 1.00 | 88.07 | B |
| ATOM | 3909 | NH2 | ARG | B | 39 | −15.240 | −6.008 | 54.947 | 1.00 | 91.89 | B |
| ATOM | 3910 | C | ARG | B | 39 | −8.582 | −4.935 | 51.083 | 1.00 | 50.25 | B |
| ATOM | 3911 | O | ARG | B | 39 | −8.462 | −5.527 | 50.009 | 1.00 | 53.48 | B |
| ATOM | 3912 | N | ASP | B | 40 | −7.932 | −5.298 | 52.181 | 1.00 | 51.47 | B |
| ATOM | 3913 | CA | ASP | B | 40 | −7.018 | −6.435 | 52.184 | 1.00 | 49.42 | B |
| ATOM | 3914 | CB | ASP | B | 40 | −7.806 | −7.743 | 52.124 | 1.00 | 113.09 | B |
| ATOM | 3915 | CG | ASP | B | 40 | −8.836 | −7.853 | 53.236 | 1.00 | 115.23 | B |
| ATOM | 3916 | OD1 | ASP | B | 40 | −8.455 | −7.696 | 54.417 | 1.00 | 112.11 | B |
| ATOM | 3917 | OD2 | ASP | B | 40 | −10.025 | −8.098 | 52.932 | 1.00 | 115.35 | B |
| ATOM | 3918 | C | ASP | B | 40 | −6.034 | −6.369 | 51.022 | 1.00 | 45.55 | B |
| ATOM | 3919 | O | ASP | B | 40 | −5.690 | −7.392 | 50.442 | 1.00 | 44.78 | B |
| ATOM | 3920 | N | ASN | B | 41 | −5.596 | −5.155 | 50.685 | 1.00 | 49.92 | B |
| ATOM | 3921 | CA | ASN | B | 41 | −4.640 | −4.899 | 49.609 | 1.00 | 45.67 | B |
| ATOM | 3922 | CB | ASN | B | 41 | −3.381 | −5.738 | 49.804 | 1.00 | 53.67 | B |
| ATOM | 3923 | CG | ASN | B | 41 | −2.161 | −5.094 | 49.177 | 1.00 | 48.36 | B |
| ATOM | 3924 | OD1 | ASN | B | 41 | −1.835 | −3.934 | 49.461 | 1.00 | 49.70 | B |
| ATOM | 3925 | ND2 | ASN | B | 41 | −1.480 | −5.839 | 48.318 | 1.00 | 44.46 | B |
| ATOM | 3926 | C | ASN | B | 41 | −5.185 | −5.125 | 48.203 | 1.00 | 48.98 | B |
| ATOM | 3927 | O | ASN | B | 41 | −4.427 | −5.387 | 47.262 | 1.00 | 50.49 | B |
| ATOM | 3928 | N | GLN | B | 42 | −6.498 | −5.010 | 48.057 | 1.00 | 39.71 | B |
| ATOM | 3929 | CA | GLN | B | 42 | −7.132 | −5.196 | 46.763 | 1.00 | 37.63 | B |
| ATOM | 3930 | CB | GLN | B | 42 | −7.883 | −6.520 | 46.724 | 1.00 | 48.06 | B |
| ATOM | 3931 | CG | GLN | B | 42 | −7.072 | −7.692 | 47.218 | 1.00 | 50.15 | B |
| ATOM | 3932 | CD | GLN | B | 42 | −7.862 | −8.983 | 47.231 | 1.00 | 49.61 | B |
| ATOM | 3933 | OE1 | GLN | B | 42 | −8.997 | −9.029 | 47.715 | 1.00 | 49.04 | B |
| ATOM | 3934 | NE2 | GLN | B | 42 | −7.258 | −10.047 | 46.710 | 1.00 | 51.37 | B |
| ATOM | 3935 | C | GLN | B | 42 | −8.115 | −4.068 | 46.539 | 1.00 | 36.52 | B |
| ATOM | 3936 | O | GLN | B | 42 | −8.692 | −3.535 | 47.495 | 1.00 | 36.86 | B |
| ATOM | 3937 | N | LEU | B | 43 | −8.300 | −3.690 | 45.278 | 1.00 | 38.81 | B |
| ATOM | 3938 | CA | LEU | B | 43 | −9.243 | −2.625 | 44.951 | 1.00 | 37.39 | B |
| ATOM | 3939 | CB | LEU | B | 43 | −8.822 | −1.915 | 43.653 | 1.00 | 22.90 | B |
| ATOM | 3940 | CG | LEU | B | 43 | −7.490 | −1.174 | 43.785 | 1.00 | 29.06 | B |
| ATOM | 3941 | CD1 | LEU | B | 43 | −7.039 | −0.683 | 42.433 | 1.00 | 29.15 | B |
| ATOM | 3942 | CD2 | LEU | B | 43 | −7.635 | −0.028 | 44.762 | 1.00 | 25.45 | B |
| ATOM | 3943 | C | LEU | B | 43 | −10.600 | −3.286 | 44.792 | 1.00 | 39.41 | B |
| ATOM | 3944 | O | LEU | B | 43 | −10.699 | −4.353 | 44.221 | 1.00 | 36.03 | B |
| ATOM | 3945 | N | VAL | B | 44 | −11.644 | −2.660 | 45.307 | 1.00 | 34.05 | B |
| ATOM | 3946 | CA | VAL | B | 44 | −12.970 | −3.245 | 45.196 | 1.00 | 33.73 | B |
| ATOM | 3947 | CB | VAL | B | 44 | −13.580 | −3.538 | 46.581 | 1.00 | 27.24 | B |
| ATOM | 3948 | CG1 | VAL | B | 44 | −14.868 | −4.298 | 46.405 | 1.00 | 28.43 | B |
| ATOM | 3949 | CG2 | VAL | B | 44 | −12.593 | −4.335 | 47.432 | 1.00 | 21.85 | B |
| ATOM | 3950 | C | VAL | B | 44 | −13.907 | −2.332 | 44.435 | 1.00 | 32.74 | B |
| ATOM | 3951 | O | VAL | B | 44 | −14.174 | −1.199 | 44.854 | 1.00 | 33.78 | B |
| ATOM | 3952 | N | VAL | B | 45 | −14.404 | −2.839 | 43.314 | 1.00 | 29.40 | B |
| ATOM | 3953 | CA | VAL | B | 45 | −15.306 | −2.095 | 42.446 | 1.00 | 35.14 | B |
| ATOM | 3954 | CB | VAL | B | 45 | −15.444 | −2.828 | 41.113 | 1.00 | 41.66 | B |
| ATOM | 3955 | CG1 | VAL | B | 45 | −16.582 | −2.246 | 40.311 | 1.00 | 44.23 | B |
| ATOM | 3956 | CG2 | VAL | B | 45 | −14.143 | −2.714 | 40.350 | 1.00 | 45.06 | B |
| ATOM | 3957 | C | VAL | B | 45 | −16.689 | −1.862 | 43.077 | 1.00 | 36.93 | B |
| ATOM | 3958 | O | VAL | B | 45 | −17.315 | −2.775 | 43.595 | 1.00 | 36.38 | B |
| ATOM | 3959 | N | PRO | B | 46 | −17.182 | −0.624 | 43.029 | 1.00 | 41.90 | B |
| ATOM | 3960 | CD | PRO | B | 46 | −16.435 | 0.594 | 42.659 | 1.00 | 49.12 | B |
| ATOM | 3961 | CA | PRO | B | 46 | −18.485 | −0.285 | 43.609 | 1.00 | 40.75 | B |
| ATOM | 3962 | CB | PRO | B | 46 | −18.274 | 1.148 | 44.072 | 1.00 | 48.48 | B |
| ATOM | 3963 | CG | PRO | B | 46 | −17.450 | 1.707 | 42.938 | 1.00 | 49.18 | B |
| ATOM | 3964 | C | PRO | B | 46 | −19.703 | −0.392 | 42.693 | 1.00 | 44.19 | B |
| ATOM | 3965 | O | PRO | B | 46 | −20.825 | −0.194 | 43.146 | 1.00 | 46.65 | B |
| ATOM | 3966 | N | SER | B | 47 | −19.499 | −0.683 | 41.412 | 1.00 | 64.81 | B |
| ATOM | 3967 | CA | SER | B | 47 | −20.635 | −0.768 | 40.499 | 1.00 | 61.93 | B |
| ATOM | 3968 | CB | SER | B | 47 | −21.141 | 0.639 | 40.193 | 1.00 | 58.46 | B |
| ATOM | 3969 | OG | SER | B | 47 | −20.134 | 1.390 | 39.540 | 1.00 | 59.67 | B |
| ATOM | 3970 | C | SER | B | 47 | −20.355 | −1.493 | 39.185 | 1.00 | 58.62 | B |
| ATOM | 3971 | O | SER | B | 47 | −19.212 | −1.585 | 38.751 | 1.00 | 55.76 | B |
| ATOM | 3972 | N | GLU | B | 48 | −21.420 | −2.003 | 38.563 | 1.00 | 42.09 | B |
| ATOM | 3973 | CA | GLU | B | 48 | −21.335 | −2.710 | 37.288 | 1.00 | 46.58 | B |
| ATOM | 3974 | CB | GLU | B | 48 | −22.737 | −3.030 | 36.754 | 1.00 | 150.28 | B |
| ATOM | 3975 | CG | GLU | B | 48 | −23.640 | −3.829 | 37.679 | 1.00 | 155.02 | B |
| ATOM | 3976 | CD | GLU | B | 48 | −23.380 | −5.321 | 37.617 | 1.00 | 159.37 | B |
| ATOM | 3977 | OE1 | GLU | B | 48 | −22.304 | −5.763 | 38.075 | 1.00 | 161.11 | B |
| ATOM | 3978 | OE2 | GLU | B | 48 | −24.255 | −6.053 | 37.105 | 1.00 | 157.29 | B |
| ATOM | 3979 | C | GLU | B | 48 | −20.677 | −1.734 | 36.331 | 1.00 | 45.17 | B |
| ATOM | 3980 | O | GLU | B | 48 | −20.830 | −0.519 | 36.486 | 1.00 | 50.96 | B |
| ATOM | 3981 | N | GLY | B | 49 | −19.940 | −2.244 | 35.348 | 1.00 | 42.47 | B |
| ATOM | 3982 | CA | GLY | B | 49 | −19.333 | −1.344 | 34.388 | 1.00 | 40.13 | B |
| ATOM | 3983 | C | GLY | B | 49 | −18.058 | −1.779 | 33.712 | 1.00 | 42.14 | B |
| ATOM | 3984 | O | GLY | B | 49 | −17.456 | −2.797 | 34.047 | 1.00 | 44.98 | B |
| ATOM | 3985 | N | LEU | B | 50 | −17.668 | −0.972 | 32.734 | 1.00 | 42.51 | B |
| ATOM | 3986 | CA | LEU | B | 50 | −16.452 | −1.166 | 31.973 | 1.00 | 43.71 | B |
| ATOM | 3987 | CB | LEU | B | 50 | −16.643 | −0.561 | 30.588 | 1.00 | 32.85 | B |
| ATOM | 3988 | CG | LEU | B | 50 | −16.625 | −1.468 | 29.359 | 1.00 | 33.98 | B |
| ATOM | 3989 | CD1 | LEU | B | 50 | −17.129 | −2.856 | 29.722 | 1.00 | 30.79 | B |
| ATOM | 3990 | CD2 | LEU | B | 50 | −17.473 | −0.833 | 28.266 | 1.00 | 34.37 | B |
| ATOM | 3991 | C | LEU | B | 50 | −15.357 | −0.409 | 32.729 | 1.00 | 40.09 | B |
| ATOM | 3992 | O | LEU | B | 50 | −15.542 | 0.755 | 33.081 | 1.00 | 38.81 | B |
| ATOM | 3993 | N | TYR | B | 51 | −14.230 | −1.057 | 33.000 | 1.00 | 41.31 | B |
| ATOM | 3994 | CA | TYR | B | 51 | −13.149 | −0.393 | 33.723 | 1.00 | 39.96 | B |
| ATOM | 3995 | CB | TYR | B | 51 | −12.985 | −0.958 | 35.132 | 1.00 | 42.17 | B |
| ATOM | 3996 | CG | TYR | B | 51 | −14.057 | −0.602 | 36.124 | 1.00 | 42.27 | B |
| ATOM | 3997 | CD1 | TYR | B | 51 | −15.320 | −1.181 | 36.064 | 1.00 | 43.13 | B |
| ATOM | 3998 | CE1 | TYR | B | 51 | −16.298 | −0.858 | 36.997 | 1.00 | 42.69 | B |
| ATOM | 3999 | CD2 | TYR | B | 51 | −13.799 | 0.305 | 37.144 | 1.00 | 43.83 | B |
| ATOM | 4000 | CE2 | TYR | B | 51 | −14.769 | 0.634 | 38.088 | 1.00 | 42.10 | B |
| ATOM | 4001 | CZ | TYR | B | 51 | −16.015 | 0.053 | 38.009 | 1.00 | 41.80 | B |
| ATOM | 4002 | OH | TYR | B | 51 | −16.983 | 0.387 | 38.930 | 1.00 | 42.03 | B |
| ATOM | 4003 | C | TYR | B | 51 | −11.806 | −0.540 | 33.044 | 1.00 | 40.14 | B |
| ATOM | 4004 | O | TYR | B | 51 | −11.498 | −1.592 | 32.453 | 1.00 | 41.03 | B |
| ATOM | 4005 | N | LEU | B | 52 | −11.009 | 0.523 | 33.125 | 1.00 | 33.63 | B |
| ATOM | 4006 | CA | LEU | B | 52 | −9.657 | 0.489 | 32.597 | 1.00 | 35.28 | B |
| ATOM | 4007 | CB | LEU | B | 52 | −9.249 | 1.854 | 32.039 | 1.00 | 49.87 | B |
| ATOM | 4008 | CG | LEU | B | 52 | −7.758 | 1.992 | 31.694 | 1.00 | 45.76 | B |
| ATOM | 4009 | CD1 | LEU | B | 52 | −7.298 | 0.855 | 30.802 | 1.00 | 47.79 | B |
| ATOM | 4010 | CD2 | LEU | B | 52 | −7.524 | 3.315 | 31.009 | 1.00 | 40.09 | B |
| ATOM | 4011 | C | LEU | B | 52 | −8.821 | 0.124 | 33.834 | 1.00 | 36.81 | B |
| ATOM | 4012 | O | LEU | B | 52 | −8.887 | 0.806 | 34.863 | 1.00 | 37.38 | B |
| ATOM | 4013 | N | ILE | B | 53 | −8.082 | −0.976 | 33.745 | 1.00 | 37.74 | B |
| ATOM | 4014 | CA | ILE | B | 53 | −7.258 | −1.448 | 34.851 | 1.00 | 34.34 | B |
| ATOM | 4015 | CB | ILE | B | 53 | −7.606 | −2.895 | 35.218 | 1.00 | 39.93 | B |
| ATOM | 4016 | CG2 | ILE | B | 53 | −6.777 | −3.345 | 36.383 | 1.00 | 40.57 | B |
| ATOM | 4017 | CG1 | ILE | B | 53 | −9.087 | −3.013 | 35.545 | 1.00 | 38.82 | B |
| ATOM | 4018 | CD1 | ILE | B | 53 | −9.503 | −4.422 | 35.901 | 1.00 | 34.64 | B |
| ATOM | 4019 | C | ILE | B | 53 | −5.812 | −1.426 | 34.399 | 1.00 | 34.40 | B |
| ATOM | 4020 | O | ILE | B | 53 | −5.501 | −1.812 | 33.258 | 1.00 | 37.48 | B |
| ATOM | 4021 | N | TYR | B | 54 | −4.921 | −0.993 | 35.279 | 1.00 | 36.86 | B |
| ATOM | 4022 | CA | TYR | B | 54 | −3.511 | −0.944 | 34.905 | 1.00 | 33.99 | B |
| ATOM | 4023 | CB | TYR | B | 54 | −3.235 | 0.324 | 34.096 | 1.00 | 27.66 | B |
| ATOM | 4024 | CG | TYR | B | 54 | −3.452 | 1.593 | 34.880 | 1.00 | 27.66 | B |
| ATOM | 4025 | CD1 | TYR | B | 54 | −2.434 | 2.126 | 35.667 | 1.00 | 27.66 | B |
| ATOM | 4026 | CE1 | TYR | B | 54 | −2.647 | 3.252 | 36.461 | 1.00 | 27.66 | B |
| ATOM | 4027 | CD2 | TYR | B | 54 | −4.698 | 2.219 | 34.899 | 1.00 | 27.66 | B |
| ATOM | 4028 | CE2 | TYR | B | 54 | −4.920 | 3.355 | 35.698 | 1.00 | 27.66 | B |
| ATOM | 4029 | CZ | TYR | B | 54 | −3.886 | 3.860 | 36.470 | 1.00 | 27.66 | B |
| ATOM | 4030 | OH | TYR | B | 54 | −4.086 | 4.983 | 37.227 | 1.00 | 27.66 | B |
| ATOM | 4031 | C | TYR | B | 54 | −2.586 | −1.001 | 36.111 | 1.00 | 32.58 | B |
| ATOM | 4032 | O | TYR | B | 54 | −2.995 | −0.723 | 37.248 | 1.00 | 31.43 | B |
| ATOM | 4033 | N | SER | B | 55 | −1.331 | −1.348 | 35.858 | 1.00 | 36.55 | B |
| ATOM | 4034 | CA | SER | B | 55 | −0.359 | −1.444 | 36.932 | 1.00 | 36.33 | B |
| ATOM | 4035 | CB | SER | B | 55 | −0.620 | −2.690 | 37.769 | 1.00 | 34.68 | B |
| ATOM | 4036 | OG | SER | B | 55 | 0.414 | −2.891 | 38.709 | 1.00 | 41.24 | B |
| ATOM | 4037 | C | SER | B | 55 | 1.047 | −1.517 | 36.397 | 1.00 | 39.31 | B |
| ATOM | 4038 | O | SER | B | 55 | 1.271 | −2.006 | 35.290 | 1.00 | 36.45 | B |
| ATOM | 4039 | N | GLN | B | 56 | 1.993 | −1.020 | 37.186 | 1.00 | 49.77 | B |
| ATOM | 4040 | CA | GLN | B | 56 | 3.393 | −1.072 | 36.812 | 1.00 | 50.47 | B |
| ATOM | 4041 | CB | GLN | B | 56 | 3.880 | 0.233 | 36.211 | 1.00 | 60.88 | B |
| ATOM | 4042 | CG | GLN | B | 56 | 5.299 | 0.076 | 35.708 | 1.00 | 62.48 | B |
| ATOM | 4043 | CD | GLN | B | 56 | 5.972 | 1.377 | 35.389 | 1.00 | 65.60 | B |
| ATOM | 4044 | OE1 | GLN | B | 56 | 6.443 | 2.077 | 36.290 | 1.00 | 65.13 | B |
| ATOM | 4045 | NE2 | GLN | B | 56 | 6.026 | 1.720 | 34.101 | 1.00 | 68.71 | B |
| ATOM | 4046 | C | GLN | B | 56 | 4.260 | −1.358 | 38.012 | 1.00 | 52.95 | B |
| ATOM | 4047 | O | GLN | B | 56 | 4.073 | −0.770 | 39.081 | 1.00 | 51.03 | B |
| ATOM | 4048 | N | VAL | B | 57 | 5.210 | −2.267 | 37.836 | 1.00 | 46.84 | B |
| ATOM | 4049 | CA | VAL | B | 57 | 6.145 | −2.589 | 38.903 | 1.00 | 41.21 | B |
| ATOM | 4050 | CB | VAL | B | 57 | 5.996 | −4.036 | 39.404 | 1.00 | 43.52 | B |
| ATOM | 4051 | CG1 | VAL | B | 57 | 4.583 | −4.263 | 39.921 | 1.00 | 44.72 | B |
| ATOM | 4052 | CG2 | VAL | B | 57 | 6.333 | −4.997 | 38.291 | 1.00 | 40.45 | B |
| ATOM | 4053 | C | VAL | B | 57 | 7.526 | −2.417 | 38.327 | 1.00 | 43.82 | B |
| ATOM | 4054 | O | VAL | B | 57 | 7.720 | −2.566 | 37.133 | 1.00 | 44.93 | B |
| ATOM | 4055 | N | LEU | B | 58 | 8.482 | −2.080 | 39.178 | 1.00 | 48.98 | B |
| ATOM | 4056 | CA | LEU | B | 58 | 9.851 | −1.903 | 38.732 | 1.00 | 47.43 | B |
| ATOM | 4057 | CB | LEU | B | 58 | 10.305 | −0.460 | 38.945 | 1.00 | 57.06 | B |
| ATOM | 4058 | CG | LEU | B | 58 | 11.394 | 0.068 | 38.009 | 1.00 | 59.08 | B |
| ATOM | 4059 | CD1 | LEU | B | 58 | 12.081 | 1.247 | 38.675 | 1.00 | 53.10 | B |
| ATOM | 4060 | CD2 | LEU | B | 58 | 12.399 | −1.019 | 37.691 | 1.00 | 56.86 | B |
| ATOM | 4061 | C | LEU | B | 58 | 10.716 | −2.820 | 39.568 | 1.00 | 50.40 | B |
| ATOM | 4062 | O | LEU | B | 58 | 10.791 | −2.661 | 40.785 | 1.00 | 52.03 | B |
| ATOM | 4063 | N | PHE | B | 59 | 11.352 | −3.789 | 38.918 | 1.00 | 47.65 | B |
| ATOM | 4064 | CA | PHE | B | 59 | 12.229 | −4.717 | 39.619 | 1.00 | 48.55 | B |
| ATOM | 4065 | CB | PHE | B | 59 | 12.131 | −6.113 | 39.023 | 1.00 | 47.79 | B |
| ATOM | 4066 | CG | PHE | B | 59 | 10.796 | −6.763 | 39.209 | 1.00 | 44.28 | B |
| ATOM | 4067 | CD1 | PHE | B | 59 | 9.897 | −6.850 | 38.148 | 1.00 | 42.72 | B |
| ATOM | 4068 | CD2 | PHE | B | 59 | 10.441 | −7.312 | 40.434 | 1.00 | 46.24 | B |
| ATOM | 4069 | CE1 | PHE | B | 59 | 8.664 | −7.479 | 38.310 | 1.00 | 48.64 | B |
| ATOM | 4070 | CE2 | PHE | B | 59 | 9.212 | −7.940 | 40.603 | 1.00 | 49.35 | B |
| ATOM | 4071 | CZ | PHE | B | 59 | 8.325 | −8.023 | 39.543 | 1.00 | 47.95 | B |
| ATOM | 4072 | C | PHE | B | 59 | 13.673 | −4.234 | 39.507 | 1.00 | 52.66 | B |
| ATOM | 4073 | O | PHE | B | 59 | 14.072 | −3.658 | 38.493 | 1.00 | 52.35 | B |
| ATOM | 4074 | N | LYS | B | 60 | 14.447 | −4.466 | 40.561 | 1.00 | 68.54 | B |
| ATOM | 4075 | CA | LYS | B | 60 | 15.847 | −4.076 | 40.600 | 1.00 | 69.89 | B |
| ATOM | 4076 | CB | LYS | B | 60 | 16.009 | −2.724 | 41.289 | 1.00 | 86.85 | B |
| ATOM | 4077 | CG | LYS | B | 60 | 17.453 | −2.296 | 41.466 | 1.00 | 93.04 | B |
| ATOM | 4078 | CD | LYS | B | 60 | 17.530 | −0.991 | 42.229 | 1.00 | 101.25 | B |
| ATOM | 4079 | CE | LYS | B | 60 | 18.964 | −0.551 | 42.441 | 1.00 | 104.00 | B |
| ATOM | 4080 | NZ | LYS | B | 60 | 19.009 | 0.770 | 43.132 | 1.00 | 104.34 | B |
| ATOM | 4081 | C | LYS | B | 60 | 16.635 | −5.131 | 41.362 | 1.00 | 69.40 | B |
| ATOM | 4082 | O | LYS | B | 60 | 16.193 | −5.626 | 42.399 | 1.00 | 72.15 | B |
| ATOM | 4083 | N | GLY | B | 61 | 17.804 | −5.477 | 40.840 | 1.00 | 81.86 | B |
| ATOM | 4084 | CA | GLY | B | 61 | 18.625 | −6.468 | 41.500 | 1.00 | 84.42 | B |
| ATOM | 4085 | C | GLY | B | 61 | 20.089 | −6.121 | 41.396 | 1.00 | 86.12 | B |
| ATOM | 4086 | O | GLY | B | 61 | 20.527 | −5.584 | 40.379 | 1.00 | 89.29 | B |
| ATOM | 4087 | N | GLN | B | 62 | 20.839 | −6.411 | 42.454 | 1.00 | 108.42 | B |
| ATOM | 4088 | CA | GLN | B | 62 | 22.272 | −6.146 | 42.475 | 1.00 | 108.73 | B |
| ATOM | 4089 | CB | GLN | B | 62 | 22.752 | −5.897 | 43.906 | 1.00 | 115.30 | B |
| ATOM | 4090 | CG | GLN | B | 62 | 22.039 | −4.755 | 44.619 | 1.00 | 119.43 | B |
| ATOM | 4091 | CD | GLN | B | 62 | 22.302 | −3.404 | 43.980 | 1.00 | 125.37 | B |
| ATOM | 4092 | OE1 | GLN | B | 62 | 21.992 | −3.183 | 42.810 | 1.00 | 124.97 | B |
| ATOM | 4093 | NE2 | GLN | B | 62 | 22.875 | −2.489 | 44.752 | 1.00 | 125.54 | B |
| ATOM | 4094 | C | GLN | B | 62 | 22.940 | −7.389 | 41.916 | 1.00 | 106.67 | B |
| ATOM | 4095 | O | GLN | B | 62 | 22.764 | −8.480 | 42.451 | 1.00 | 108.18 | B |
| ATOM | 4096 | N | GLY | B | 63 | 23.690 | −7.230 | 40.831 | 1.00 | 89.45 | B |
| ATOM | 4097 | CA | GLY | B | 63 | 24.361 | −8.370 | 40.232 | 1.00 | 87.44 | B |
| ATOM | 4098 | C | GLY | B | 63 | 23.380 | −9.480 | 39.928 | 1.00 | 85.52 | B |
| ATOM | 4099 | O | GLY | B | 63 | 22.174 | −9.274 | 40.009 | 1.00 | 83.56 | B |
| ATOM | 4100 | N | CYS | B | 64 | 23.885 | −10.663 | 39.599 | 1.00 | 144.85 | B |
| ATOM | 4101 | CA | CYS | B | 64 | 23.007 | −11.778 | 39.274 | 1.00 | 149.09 | B |
| ATOM | 4102 | C | CYS | B | 64 | 23.003 | −12.971 | 40.221 | 1.00 | 155.18 | B |
| ATOM | 4103 | O | CYS | B | 64 | 24.041 | −13.373 | 40.746 | 1.00 | 158.09 | B |
| ATOM | 4104 | CB | CYS | B | 64 | 23.295 | −12.260 | 37.859 | 1.00 | 144.61 | B |
| ATOM | 4105 | SG | CYS | B | 64 | 22.485 | −11.211 | 36.622 | 1.00 | 145.82 | B |
| ATOM | 4106 | N | PRO | B | 65 | 21.812 | −13.550 | 40.453 | 1.00 | 124.01 | B |
| ATOM | 4107 | CD | PRO | B | 65 | 20.505 | −12.956 | 40.129 | 1.00 | 147.67 | B |
| ATOM | 4108 | CA | PRO | B | 65 | 21.641 | −14.707 | 41.334 | 1.00 | 126.35 | B |
| ATOM | 4109 | CB | PRO | B | 65 | 20.126 | −14.766 | 41.541 | 1.00 | 150.20 | B |
| ATOM | 4110 | CG | PRO | B | 65 | 19.688 | −13.350 | 41.325 | 1.00 | 149.93 | B |
| ATOM | 4111 | C | PRO | B | 65 | 22.170 | −15.980 | 40.687 | 1.00 | 129.36 | B |
| ATOM | 4112 | O | PRO | B | 65 | 23.075 | −15.942 | 39.852 | 1.00 | 129.63 | B |
| ATOM | 4113 | N | SER | B | 66 | 21.586 | −17.106 | 41.075 | 1.00 | 171.09 | B |
| ATOM | 4114 | CA | SER | B | 66 | 21.982 | −18.400 | 40.544 | 1.00 | 174.69 | B |
| ATOM | 4115 | CB | SER | B | 66 | 21.950 | −19.452 | 41.657 | 1.00 | 158.34 | B |
| ATOM | 4116 | OG | SER | B | 66 | 22.304 | −20.734 | 41.167 | 1.00 | 155.50 | B |
| ATOM | 4117 | C | SER | B | 66 | 21.046 | −18.812 | 39.416 | 1.00 | 175.24 | B |
| ATOM | 4118 | O | SER | B | 66 | 20.081 | −18.110 | 39.110 | 1.00 | 168.66 | B |
| ATOM | 4119 | N | THR | B | 67 | 21.349 | −19.954 | 38.807 | 1.00 | 200.84 | B |
| ATOM | 4120 | CA | THR | B | 67 | 20.565 | −20.518 | 37.711 | 1.00 | 200.84 | B |
| ATOM | 4121 | CB | THR | B | 67 | 19.554 | −21.571 | 38.233 | 1.00 | 176.00 | B |
| ATOM | 4122 | OG1 | THR | B | 67 | 20.243 | −22.545 | 39.027 | 1.00 | 176.00 | B |
| ATOM | 4123 | CG2 | THR | B | 67 | 18.866 | −22.276 | 37.066 | 1.00 | 176.00 | B |
| ATOM | 4124 | C | THR | B | 67 | 19.798 | −19.484 | 36.891 | 1.00 | 200.84 | B |
| ATOM | 4125 | O | THR | B | 67 | 20.357 | −18.839 | 36.004 | 1.00 | 200.84 | B |
| ATOM | 4126 | N | HIS | B | 68 | 18.514 | −19.328 | 37.199 | 1.00 | 158.58 | B |
| ATOM | 4127 | CA | HIS | B | 68 | 17.668 | −18.395 | 36.474 | 1.00 | 153.70 | B |
| ATOM | 4128 | CB | HIS | B | 68 | 17.039 | −19.114 | 35.279 | 1.00 | 184.11 | B |
| ATOM | 4129 | CG | HIS | B | 68 | 16.589 | −18.199 | 34.185 | 1.00 | 184.11 | B |
| ATOM | 4130 | CD2 | HIS | B | 68 | 15.367 | −17.992 | 33.641 | 1.00 | 184.11 | B |
| ATOM | 4131 | ND1 | HIS | B | 68 | 17.456 | −17.375 | 33.502 | 1.00 | 184.11 | B |
| ATOM | 4132 | CE1 | HIS | B | 68 | 16.789 | −16.700 | 32.584 | 1.00 | 184.11 | B |
| ATOM | 4133 | NE2 | HIS | B | 68 | 15.519 | −17.056 | 32.647 | 1.00 | 184.11 | B |
| ATOM | 4134 | C | HIS | B | 68 | 16.573 | −17.846 | 37.386 | 1.00 | 149.53 | B |
| ATOM | 4135 | O | HIS | B | 68 | 16.037 | −18.565 | 38.229 | 1.00 | 148.98 | B |
| ATOM | 4136 | N | VAL | B | 69 | 16.248 | −16.569 | 37.214 | 1.00 | 111.57 | B |
| ATOM | 4137 | CA | VAL | B | 69 | 15.211 | −15.920 | 38.012 | 1.00 | 111.30 | B |
| ATOM | 4138 | CB | VAL | B | 69 | 15.784 | −14.738 | 38.825 | 1.00 | 101.65 | B |
| ATOM | 4139 | CG1 | VAL | B | 69 | 14.694 | −14.113 | 39.672 | 1.00 | 101.41 | B |
| ATOM | 4140 | CG2 | VAL | B | 69 | 16.924 | −15.217 | 39.707 | 1.00 | 99.91 | B |
| ATOM | 4141 | C | VAL | B | 69 | 14.104 | −15.392 | 37.105 | 1.00 | 110.89 | B |
| ATOM | 4142 | O | VAL | B | 69 | 14.375 | −14.767 | 36.079 | 1.00 | 114.45 | B |
| ATOM | 4143 | N | LEU | B | 70 | 12.858 | −15.659 | 37.482 | 1.00 | 77.62 | B |
| ATOM | 4144 | CA | LEU | B | 70 | 11.711 | −15.196 | 36.708 | 1.00 | 72.84 | B |
| ATOM | 4145 | CB | LEU | B | 70 | 10.774 | −16.354 | 36.357 | 1.00 | 62.30 | B |
| ATOM | 4146 | CG | LEU | B | 70 | 11.287 | −17.404 | 35.366 | 1.00 | 64.00 | B |
| ATOM | 4147 | CD1 | LEU | B | 70 | 10.183 | −18.387 | 35.077 | 1.00 | 59.04 | B |
| ATOM | 4148 | CD2 | LEU | B | 70 | 11.749 | −16.745 | 34.072 | 1.00 | 64.60 | B |
| ATOM | 4149 | C | LEU | B | 70 | 10.925 | −14.155 | 37.478 | 1.00 | 73.88 | B |
| ATOM | 4150 | O | LEU | B | 70 | 10.660 | −14.317 | 38.666 | 1.00 | 73.99 | B |
| ATOM | 4151 | N | LEU | B | 71 | 10.559 | −13.083 | 36.789 | 1.00 | 64.03 | B |
| ATOM | 4152 | CA | LEU | B | 71 | 9.787 | −12.009 | 37.396 | 1.00 | 63.97 | B |
| ATOM | 4153 | CB | LEU | B | 71 | 10.473 | −10.659 | 37.156 | 1.00 | 51.73 | B |
| ATOM | 4154 | CG | LEU | B | 71 | 11.891 | −10.517 | 37.710 | 1.00 | 49.40 | B |
| ATOM | 4155 | CD1 | LEU | B | 71 | 12.460 | −9.179 | 37.298 | 1.00 | 45.55 | B |
| ATOM | 4156 | CD2 | LEU | B | 71 | 11.866 | −10.657 | 39.220 | 1.00 | 49.52 | B |
| ATOM | 4157 | C | LEU | B | 71 | 8.407 | −12.006 | 36.752 | 1.00 | 68.00 | B |
| ATOM | 4158 | O | LEU | B | 71 | 8.288 | −11.979 | 35.524 | 1.00 | 67.49 | B |
| ATOM | 4159 | N | THR | B | 72 | 7.367 | −12.058 | 37.577 | 1.00 | 41.22 | B |
| ATOM | 4160 | CA | THR | B | 72 | 6.008 | −12.049 | 37.051 | 1.00 | 38.65 | B |
| ATOM | 4161 | CB | THR | B | 72 | 5.281 | −13.402 | 37.254 | 1.00 | 55.66 | B |
| ATOM | 4162 | OG1 | THR | B | 72 | 4.853 | −13.526 | 38.614 | 1.00 | 58.19 | B |
| ATOM | 4163 | CG2 | THR | B | 72 | 6.197 | −14.554 | 36.917 | 1.00 | 53.38 | B |
| ATOM | 4164 | C | THR | B | 72 | 5.170 | −10.962 | 37.701 | 1.00 | 41.28 | B |
| ATOM | 4165 | O | THR | B | 72 | 5.292 | −10.672 | 38.900 | 1.00 | 42.32 | B |
| ATOM | 4166 | N | HIS | B | 73 | 4.326 | −10.348 | 36.886 | 1.00 | 38.86 | B |
| ATOM | 4167 | CA | HIS | B | 73 | 3.432 | −9.304 | 37.348 | 1.00 | 39.31 | B |
| ATOM | 4168 | CB | HIS | B | 73 | 3.823 | −7.965 | 36.744 | 1.00 | 33.00 | B |
| ATOM | 4169 | CG | HIS | B | 73 | 3.023 | −6.820 | 37.269 | 1.00 | 35.87 | B |
| ATOM | 4170 | CD2 | HIS | B | 73 | 2.265 | −6.698 | 38.386 | 1.00 | 37.63 | B |
| ATOM | 4171 | ND1 | HIS | B | 73 | 2.966 | −5.602 | 36.632 | 1.00 | 40.48 | B |
| ATOM | 4172 | CE1 | HIS | B | 73 | 2.207 | −4.778 | 37.332 | 1.00 | 39.02 | B |
| ATOM | 4173 | NE2 | HIS | B | 73 | 1.770 | −5.418 | 38.401 | 1.00 | 39.19 | B |
| ATOM | 4174 | C | HIS | B | 73 | 2.076 | −9.722 | 36.817 | 1.00 | 38.39 | B |
| ATOM | 4175 | O | HIS | B | 73 | 1.958 | −10.110 | 35.656 | 1.00 | 34.25 | B |
| ATOM | 4176 | N | THR | B | 74 | 1.048 | −9.668 | 37.645 | 1.00 | 44.19 | B |
| ATOM | 4177 | CA | THR | B | 74 | −0.247 | −10.086 | 37.152 | 1.00 | 50.78 | B |
| ATOM | 4178 | CB | THR | B | 74 | −0.333 | −11.650 | 37.132 | 1.00 | 50.78 | B |
| ATOM | 4179 | OG1 | THR | B | 74 | −1.572 | −12.081 | 37.704 | 1.00 | 53.29 | B |
| ATOM | 4180 | CG2 | THR | B | 74 | 0.822 | −12.271 | 37.900 | 1.00 | 55.61 | B |
| ATOM | 4181 | C | THR | B | 74 | −1.421 | −9.479 | 37.919 | 1.00 | 49.63 | B |
| ATOM | 4182 | O | THR | B | 74 | −1.383 | −9.352 | 39.140 | 1.00 | 45.21 | B |
| ATOM | 4183 | N | ILE | B | 75 | −2.456 | −9.079 | 37.186 | 1.00 | 35.07 | B |
| ATOM | 4184 | CA | ILE | B | 75 | −3.651 | −8.502 | 37.790 | 1.00 | 36.37 | B |
| ATOM | 4185 | CB | ILE | B | 75 | −4.109 | −7.235 | 37.037 | 1.00 | 41.98 | B |
| ATOM | 4186 | CG2 | ILE | B | 75 | −5.425 | −6.723 | 37.616 | 1.00 | 41.47 | B |
| ATOM | 4187 | CG1 | ILE | B | 75 | −3.020 | −6.164 | 37.129 | 1.00 | 39.14 | B |
| ATOM | 4188 | CD1 | ILE | B | 75 | −3.377 | −4.859 | 36.455 | 1.00 | 42.43 | B |
| ATOM | 4189 | C | ILE | B | 75 | −4.748 | −9.552 | 37.723 | 1.00 | 39.51 | B |
| ATOM | 4190 | O | ILE | B | 75 | −5.087 | −10.037 | 36.645 | 1.00 | 36.28 | B |
| ATOM | 4191 | N | SER | B | 76 | −5.291 | −9.902 | 38.882 | 1.00 | 44.59 | B |
| ATOM | 4192 | CA | SER | B | 76 | −6.331 | −10.915 | 38.978 | 1.00 | 45.67 | B |
| ATOM | 4193 | CB | SER | B | 76 | −5.987 | −11.943 | 40.052 | 1.00 | 59.42 | B |
| ATOM | 4194 | OG | SER | B | 76 | −4.751 | −12.566 | 39.800 | 1.00 | 68.71 | B |
| ATOM | 4195 | C | SER | B | 76 | −7.658 | −10.328 | 39.346 | 1.00 | 41.67 | B |
| ATOM | 4196 | O | SER | B | 76 | −7.734 | −9.277 | 39.972 | 1.00 | 37.82 | B |
| ATOM | 4197 | N | ARG | B | 77 | −8.713 | −11.027 | 38.958 | 1.00 | 37.42 | B |
| ATOM | 4198 | CA | ARG | B | 77 | −10.064 | −10.620 | 39.314 | 1.00 | 42.60 | B |
| ATOM | 4199 | CB | ARG | B | 77 | −10.932 | −10.363 | 38.071 | 1.00 | 35.77 | B |
| ATOM | 4200 | CG | ARG | B | 77 | −12.426 | −10.491 | 38.373 | 1.00 | 37.95 | B |
| ATOM | 4201 | CD | ARG | B | 77 | −13.299 | −9.728 | 37.415 | 1.00 | 38.82 | B |
| ATOM | 4202 | NE | ARG | B | 77 | −14.047 | −10.577 | 36.489 | 1.00 | 41.26 | B |
| ATOM | 4203 | CZ | ARG | B | 77 | −15.374 | −10.539 | 36.353 | 1.00 | 44.69 | B |
| ATOM | 4204 | NH1 | ARG | B | 77 | −15.986 | −11.330 | 35.483 | 1.00 | 44.78 | B |
| ATOM | 4205 | NH2 | ARG | B | 77 | −16.098 | −9.720 | 37.102 | 1.00 | 44.55 | B |
| ATOM | 4206 | C | ARG | B | 77 | −10.678 | −11.760 | 40.116 | 1.00 | 40.94 | B |
| ATOM | 4207 | O | ARG | B | 77 | −10.547 | −12.928 | 39.751 | 1.00 | 46.69 | B |
| ATOM | 4208 | N | ILE | B | 78 | −11.296 | −11.435 | 41.236 | 1.00 | 39.41 | B |
| ATOM | 4209 | CA | ILE | B | 78 | −11.978 | −12.456 | 41.999 | 1.00 | 38.10 | B |
| ATOM | 4210 | CB | ILE | B | 78 | −11.369 | −12.661 | 43.449 | 1.00 | 50.84 | B |
| ATOM | 4211 | CG2 | ILE | B | 78 | −10.017 | −11.990 | 43.569 | 1.00 | 51.34 | B |
| ATOM | 4212 | CG1 | ILE | B | 78 | −12.321 | −12.167 | 44.521 | 1.00 | 52.51 | B |
| ATOM | 4213 | CD1 | ILE | B | 78 | −13.529 | −13.055 | 44.711 | 1.00 | 52.83 | B |
| ATOM | 4214 | C | ILE | B | 78 | −13.403 | −11.883 | 41.969 | 1.00 | 40.27 | B |
| ATOM | 4215 | O | ILE | B | 78 | −13.737 | −10.910 | 42.664 | 1.00 | 38.82 | B |
| ATOM | 4216 | N | ALA | B | 79 | −14.222 | −12.460 | 41.090 | 1.00 | 39.45 | B |
| ATOM | 4217 | CA | ALA | B | 79 | −15.587 | −12.004 | 40.906 | 1.00 | 36.05 | B |
| ATOM | 4218 | CB | ALA | B | 79 | −16.126 | −12.543 | 39.588 | 1.00 | 72.90 | B |
| ATOM | 4219 | C | ALA | B | 79 | −16.540 | −12.356 | 42.045 | 1.00 | 37.25 | B |
| ATOM | 4220 | O | ALA | B | 79 | −16.370 | −13.362 | 42.738 | 1.00 | 37.45 | B |
| ATOM | 4221 | N | VAL | B | 80 | −17.555 | −11.516 | 42.229 | 1.00 | 56.02 | B |
| ATOM | 4222 | CA | VAL | B | 80 | −18.550 | −11.750 | 43.268 | 1.00 | 58.51 | B |
| ATOM | 4223 | CB | VAL | B | 80 | −19.562 | −10.594 | 43.354 | 1.00 | 57.33 | B |
| ATOM | 4224 | CG1 | VAL | B | 80 | −20.602 | −10.896 | 44.412 | 1.00 | 55.73 | B |
| ATOM | 4225 | CG2 | VAL | B | 80 | −18.860 | −9.312 | 43.678 | 1.00 | 59.03 | B |
| ATOM | 4226 | C | VAL | B | 80 | −19.316 | −13.029 | 42.947 | 1.00 | 63.06 | B |
| ATOM | 4227 | O | VAL | B | 80 | −19.732 | −13.750 | 43.838 | 1.00 | 61.65 | B |
| ATOM | 4228 | N | SER | B | 81 | −19.502 | −13.307 | 41.666 | 1.00 | 66.61 | B |
| ATOM | 4229 | CA | SER | B | 81 | −20.222 | −14.502 | 41.253 | 1.00 | 69.47 | B |
| ATOM | 4230 | CB | SER | B | 81 | −21.038 | −14.208 | 39.995 | 1.00 | 50.24 | B |
| ATOM | 4231 | OG | SER | B | 81 | −22.027 | −13.243 | 40.274 | 1.00 | 53.28 | B |
| ATOM | 4232 | C | SER | B | 81 | −19.330 | −15.729 | 41.013 | 1.00 | 73.65 | B |
| ATOM | 4233 | O | SER | B | 81 | −19.813 | −16.757 | 40.532 | 1.00 | 74.17 | B |
| ATOM | 4234 | N | TYR | B | 82 | −18.042 | −15.624 | 41.349 | 1.00 | 43.47 | B |
| ATOM | 4235 | CA | TYR | B | 82 | −17.098 | −16.732 | 41.176 | 1.00 | 45.51 | B |
| ATOM | 4236 | CB | TYR | B | 82 | −16.778 | −16.931 | 39.694 | 1.00 | 34.66 | B |
| ATOM | 4237 | CG | TYR | B | 82 | −16.008 | −18.201 | 39.403 | 1.00 | 37.86 | B |
| ATOM | 4238 | CD1 | TYR | B | 82 | −14.765 | −18.159 | 38.779 | 1.00 | 40.15 | B |
| ATOM | 4239 | CE1 | TYR | B | 82 | −14.036 | −19.325 | 38.551 | 1.00 | 41.94 | B |
| ATOM | 4240 | CD2 | TYR | B | 82 | −16.511 | −19.446 | 39.787 | 1.00 | 39.95 | B |
| ATOM | 4241 | CE2 | TYR | B | 82 | −15.788 | −20.616 | 39.562 | 1.00 | 41.42 | B |
| ATOM | 4242 | CZ | TYR | B | 82 | −14.549 | −20.546 | 38.947 | 1.00 | 42.11 | B |
| ATOM | 4243 | OH | TYR | B | 82 | −13.809 | −21.688 | 38.750 | 1.00 | 40.53 | B |
| ATOM | 4244 | C | TYR | B | 82 | −15.813 | −16.443 | 41.942 | 1.00 | 44.94 | B |
| ATOM | 4245 | O | TYR | B | 82 | −14.785 | −16.133 | 41.351 | 1.00 | 45.12 | B |
| ATOM | 4246 | N | GLN | B | 83 | −15.895 | −16.551 | 43.265 | 1.00 | 59.45 | B |
| ATOM | 4247 | CA | GLN | B | 83 | −14.775 | −16.283 | 44.167 | 1.00 | 59.34 | B |
| ATOM | 4248 | CB | GLN | B | 83 | −15.176 | −16.575 | 45.612 | 1.00 | 73.46 | B |
| ATOM | 4249 | CG | GLN | B | 83 | −15.591 | −15.373 | 46.434 | 1.00 | 77.73 | B |
| ATOM | 4250 | CD | GLN | B | 83 | −16.985 | −14.895 | 46.107 | 1.00 | 78.77 | B |
| ATOM | 4251 | OE1 | GLN | B | 83 | −17.567 | −14.107 | 46.846 | 1.00 | 77.79 | B |
| ATOM | 4252 | NE2 | GLN | B | 83 | −17.530 | −15.367 | 44.993 | 1.00 | 76.27 | B |
| ATOM | 4253 | C | GLN | B | 83 | −13.480 | −17.029 | 43.906 | 1.00 | 57.52 | B |
| ATOM | 4254 | O | GLN | B | 83 | −13.129 | −17.917 | 44.664 | 1.00 | 54.97 | B |
| ATOM | 4255 | N | THR | B | 84 | −12.751 | −16.673 | 42.862 | 1.00 | 56.77 | B |
| ATOM | 4256 | CA | THR | B | 84 | −11.488 | −17.346 | 42.590 | 1.00 | 49.14 | B |
| ATOM | 4257 | CB | THR | B | 84 | −11.669 | −18.646 | 41.784 | 1.00 | 57.77 | B |
| ATOM | 4258 | OG1 | THR | B | 84 | −12.646 | −19.478 | 42.415 | 1.00 | 58.32 | B |
| ATOM | 4259 | CG2 | THR | B | 84 | −10.355 | −19.393 | 41.700 | 1.00 | 60.52 | B |
| ATOM | 4260 | C | THR | B | 84 | −10.648 | −16.429 | 41.748 | 1.00 | 45.65 | B |
| ATOM | 4261 | O | THR | B | 84 | −11.151 | −15.791 | 40.825 | 1.00 | 46.96 | B |
| ATOM | 4262 | N | LYS | B | 85 | −9.366 | −16.355 | 42.057 | 1.00 | 36.28 | B |
| ATOM | 4263 | CA | LYS | B | 85 | −8.485 | −15.515 | 41.279 | 1.00 | 36.14 | B |
| ATOM | 4264 | CB | LYS | B | 85 | −7.075 | −15.542 | 41.859 | 1.00 | 69.83 | B |
| ATOM | 4265 | CG | LYS | B | 85 | −6.770 | −14.463 | 42.866 | 1.00 | 66.77 | B |
| ATOM | 4266 | CD | LYS | B | 85 | −5.281 | −14.492 | 43.182 | 1.00 | 65.17 | B |
| ATOM | 4267 | CE | LYS | B | 85 | −4.859 | −13.316 | 44.051 | 1.00 | 63.47 | B |
| ATOM | 4268 | NZ | LYS | B | 85 | −5.543 | −13.319 | 45.374 | 1.00 | 61.72 | B |
| ATOM | 4269 | C | LYS | B | 85 | −8.426 | −16.027 | 39.843 | 1.00 | 35.94 | B |
| ATOM | 4270 | O | LYS | B | 85 | −8.146 | −17.199 | 39.618 | 1.00 | 36.39 | B |
| ATOM | 4271 | N | VAL | B | 86 | −8.733 | −15.166 | 38.880 | 1.00 | 33.30 | B |
| ATOM | 4272 | CA | VAL | B | 86 | −8.613 | −15.527 | 37.473 | 1.00 | 36.19 | B |
| ATOM | 4273 | CB | VAL | B | 86 | −9.986 | −15.643 | 36.734 | 1.00 | 35.45 | B |
| ATOM | 4274 | CG1 | VAL | B | 86 | −10.801 | −16.756 | 37.332 | 1.00 | 42.19 | B |
| ATOM | 4275 | CG2 | VAL | B | 86 | −10.757 | −14.355 | 36.830 | 1.00 | 36.72 | B |
| ATOM | 4276 | C | VAL | B | 86 | −7.774 | −14.380 | 36.908 | 1.00 | 36.86 | B |
| ATOM | 4277 | O | VAL | B | 86 | −8.048 | −13.207 | 37.163 | 1.00 | 41.55 | B |
| ATOM | 4278 | N | ASN | B | 87 | −6.723 | −14.713 | 36.179 | 1.00 | 56.55 | B |
| ATOM | 4279 | CA | ASN | B | 87 | −5.858 | −13.686 | 35.630 | 1.00 | 53.23 | B |
| ATOM | 4280 | CB | ASN | B | 87 | −4.552 | −14.319 | 35.161 | 1.00 | 65.66 | B |
| ATOM | 4281 | CG | ASN | B | 87 | −3.726 | −14.851 | 36.305 | 1.00 | 65.96 | B |
| ATOM | 4282 | OD1 | ASN | B | 87 | −4.049 | −14.629 | 37.475 | 1.00 | 68.57 | B |
| ATOM | 4283 | ND2 | ASN | B | 87 | −2.646 | −15.547 | 35.979 | 1.00 | 62.92 | B |
| ATOM | 4284 | C | ASN | B | 87 | −6.463 | −12.874 | 34.490 | 1.00 | 52.05 | B |
| ATOM | 4285 | O | ASN | B | 87 | −6.989 | −13.443 | 33.532 | 1.00 | 50.63 | B |
| ATOM | 4286 | N | LEU | B | 88 | −6.376 | −11.544 | 34.588 | 1.00 | 43.46 | B |
| ATOM | 4287 | CA | LEU | B | 88 | −6.888 | −10.664 | 33.536 | 1.00 | 42.45 | B |
| ATOM | 4288 | CB | LEU | B | 88 | −7.559 | −9.441 | 34.125 | 1.00 | 35.70 | B |
| ATOM | 4289 | CG | LEU | B | 88 | −8.777 | −9.670 | 34.995 | 1.00 | 32.63 | B |
| ATOM | 4290 | CD1 | LEU | B | 88 | −9.326 | −8.326 | 35.404 | 1.00 | 33.40 | B |
| ATOM | 4291 | CD2 | LEU | B | 88 | −9.814 | −10.465 | 34.228 | 1.00 | 31.20 | B |
| ATOM | 4292 | C | LEU | B | 88 | −5.738 | −10.187 | 32.671 | 1.00 | 41.69 | B |
| ATOM | 4293 | O | LEU | B | 88 | −5.845 | −10.146 | 31.447 | 1.00 | 40.16 | B |
| ATOM | 4294 | N | LEU | B | 89 | −4.641 | −9.816 | 33.331 | 1.00 | 47.60 | B |
| ATOM | 4295 | CA | LEU | B | 89 | −3.442 | −9.327 | 32.657 | 1.00 | 46.52 | B |
| ATOM | 4296 | CB | LEU | B | 89 | −3.380 | −7.807 | 32.755 | 1.00 | 34.01 | B |
| ATOM | 4297 | CG | LEU | B | 89 | −4.634 | −7.065 | 32.294 | 1.00 | 37.99 | B |
| ATOM | 4298 | CD1 | LEU | B | 89 | −4.634 | −5.683 | 32.893 | 1.00 | 37.72 | B |
| ATOM | 4299 | CD2 | LEU | B | 89 | −4.676 | −6.978 | 30.784 | 1.00 | 37.11 | B |
| ATOM | 4300 | C | LEU | B | 89 | −2.233 | −9.929 | 33.348 | 1.00 | 48.19 | B |
| ATOM | 4301 | O | LEU | B | 89 | −2.204 | −10.027 | 34.579 | 1.00 | 48.91 | B |
| ATOM | 4302 | N | SER | B | 90 | −1.236 | −10.324 | 32.565 | 1.00 | 50.76 | B |
| ATOM | 4303 | CA | SER | B | 90 | −0.043 | −10.931 | 33.133 | 1.00 | 50.10 | B |
| ATOM | 4304 | CB | SER | B | 90 | −0.325 | −12.385 | 33.498 | 1.00 | 61.74 | B |
| ATOM | 4305 | OG | SER | B | 90 | 0.838 | −13.006 | 34.007 | 1.00 | 65.96 | B |
| ATOM | 4306 | C | SER | B | 90 | 1.142 | −10.897 | 32.194 | 1.00 | 48.73 | B |
| ATOM | 4307 | O | SER | B | 90 | 0.989 | −11.039 | 30.983 | 1.00 | 48.55 | B |
| ATOM | 4308 | N | ALA | B | 91 | 2.330 | −10.727 | 32.760 | 1.00 | 44.50 | B |
| ATOM | 4309 | CA | ALA | B | 91 | 3.546 | −10.701 | 31.968 | 1.00 | 42.59 | B |
| ATOM | 4310 | CB | ALA | B | 91 | 3.886 | −9.282 | 31.595 | 1.00 | 38.37 | B |
| ATOM | 4311 | C | ALA | B | 91 | 4.695 | −11.339 | 32.744 | 1.00 | 46.56 | B |
| ATOM | 4312 | O | ALA | B | 91 | 4.664 | −11.420 | 33.974 | 1.00 | 48.43 | B |
| ATOM | 4313 | N | ILE | B | 92 | 5.706 | −11.797 | 32.018 | 1.00 | 57.88 | B |
| ATOM | 4314 | CA | ILE | B | 92 | 6.859 | −12.439 | 32.632 | 1.00 | 57.86 | B |
| ATOM | 4315 | CB | ILE | B | 92 | 6.882 | −13.944 | 32.316 | 1.00 | 45.50 | B |
| ATOM | 4316 | CG2 | ILE | B | 92 | 8.016 | −14.608 | 33.071 | 1.00 | 51.57 | B |
| ATOM | 4317 | CG1 | ILE | B | 92 | 5.542 | −14.578 | 32.700 | 1.00 | 43.61 | B |
| ATOM | 4318 | CD1 | ILE | B | 92 | 5.415 | −16.022 | 32.312 | 1.00 | 49.48 | B |
| ATOM | 4319 | C | ILE | B | 92 | 8.113 | −11.816 | 32.060 | 1.00 | 57.69 | B |
| ATOM | 4320 | O | ILE | B | 92 | 8.127 | −11.427 | 30.900 | 1.00 | 58.77 | B |
| ATOM | 4321 | N | LYS | B | 93 | 9.164 | −11.724 | 32.866 | 1.00 | 70.11 | B |
| ATOM | 4322 | CA | LYS | B | 93 | 10.416 | −11.137 | 32.403 | 1.00 | 70.76 | B |
| ATOM | 4323 | CB | LYS | B | 93 | 10.452 | −9.644 | 32.744 | 1.00 | 80.23 | B |
| ATOM | 4324 | CG | LYS | B | 93 | 11.240 | −8.797 | 31.759 | 1.00 | 83.92 | B |
| ATOM | 4325 | CD | LYS | B | 93 | 10.763 | −7.343 | 31.779 | 1.00 | 83.69 | B |
| ATOM | 4326 | CE | LYS | B | 93 | 11.432 | −6.504 | 30.683 | 1.00 | 83.59 | B |
| ATOM | 4327 | NZ | LYS | B | 93 | 10.958 | −5.081 | 30.642 | 1.00 | 86.16 | B |
| ATOM | 4328 | C | LYS | B | 93 | 11.595 | −11.862 | 33.042 | 1.00 | 72.46 | B |
| ATOM | 4329 | O | LYS | B | 93 | 11.581 | −12.151 | 34.238 | 1.00 | 70.38 | B |
| ATOM | 4330 | N | SER | B | 94 | 12.606 | −12.163 | 32.231 | 1.00 | 85.70 | B |
| ATOM | 4331 | CA | SER | B | 94 | 13.805 | −12.869 | 32.687 | 1.00 | 88.15 | B |
| ATOM | 4332 | CB | SER | B | 94 | 14.124 | −14.027 | 31.736 | 1.00 | 94.33 | B |
| ATOM | 4333 | OG | SER | B | 94 | 14.321 | −13.566 | 30.407 | 1.00 | 96.76 | B |
| ATOM | 4334 | C | SER | B | 94 | 14.994 | −11.925 | 32.742 | 1.00 | 90.74 | B |
| ATOM | 4335 | O | SER | B | 94 | 15.666 | −11.706 | 31.737 | 1.00 | 92.01 | B |
| ATOM | 4336 | N | PRO | B | 95 | 15.287 | −11.375 | 33.929 | 1.00 | 75.32 | B |
| ATOM | 4337 | CD | PRO | B | 95 | 14.698 | −11.756 | 35.226 | 1.00 | 106.72 | B |
| ATOM | 4338 | CA | PRO | B | 95 | 16.401 | −10.441 | 34.120 | 1.00 | 79.26 | B |
| ATOM | 4339 | CB | PRO | B | 95 | 16.229 | −10.004 | 35.569 | 1.00 | 109.66 | B |
| ATOM | 4340 | CG | PRO | B | 95 | 15.731 | −11.258 | 36.217 | 1.00 | 107.11 | B |
| ATOM | 4341 | C | PRO | B | 95 | 17.762 | −11.070 | 33.863 | 1.00 | 83.69 | B |
| ATOM | 4342 | O | PRO | B | 95 | 18.386 | −10.842 | 32.824 | 1.00 | 87.12 | B |
| ATOM | 4343 | N | CYS | B | 96 | 18.223 | −11.858 | 34.824 | 1.00 | 178.76 | B |
| ATOM | 4344 | CA | CYS | B | 96 | 19.504 | −12.521 | 34.700 | 1.00 | 178.55 | B |
| ATOM | 4345 | C | CYS | B | 96 | 19.368 | −13.755 | 33.831 | 1.00 | 179.65 | B |
| ATOM | 4346 | O | CYS | B | 96 | 18.780 | −14.757 | 34.240 | 1.00 | 179.95 | B |
| ATOM | 4347 | CB | CYS | B | 96 | 20.032 | −12.902 | 36.078 | 1.00 | 189.31 | B |
| ATOM | 4348 | SG | CYS | B | 96 | 20.521 | −11.460 | 37.072 | 1.00 | 189.31 | B |
| ATOM | 4349 | N | GLN | B | 97 | 19.907 | −13.661 | 32.621 | 1.00 | 200.84 | B |
| ATOM | 4350 | CA | GLN | B | 97 | 19.872 | −14.758 | 31.666 | 1.00 | 200.84 | B |
| ATOM | 4351 | CB | GLN | B | 97 | 20.150 | −14.223 | 30.256 | 1.00 | 148.28 | B |
| ATOM | 4352 | CG | GLN | B | 97 | 19.257 | −13.058 | 29.854 | 1.00 | 148.28 | B |
| ATOM | 4353 | CD | GLN | B | 97 | 19.803 | −12.282 | 28.668 | 1.00 | 148.28 | B |
| ATOM | 4354 | OE1 | GLN | B | 97 | 20.006 | −12.836 | 27.588 | 1.00 | 148.28 | B |
| ATOM | 4355 | NE2 | GLN | B | 97 | 20.043 | −10.991 | 28.867 | 1.00 | 148.28 | B |
| ATOM | 4356 | C | GLN | B | 97 | 20.939 | −15.779 | 32.062 | 1.00 | 200.84 | B |
| ATOM | 4357 | O | GLN | B | 97 | 21.675 | −16.279 | 31.210 | 1.00 | 200.84 | B |
| ATOM | 4358 | N | ARG | B | 98 | 21.017 | −16.070 | 33.361 | 1.00 | 200.81 | B |
| ATOM | 4359 | CA | ARG | B | 98 | 21.983 | −17.022 | 33.909 | 1.00 | 200.81 | B |
| ATOM | 4360 | CB | ARG | B | 98 | 21.939 | −18.333 | 33.106 | 1.00 | 199.41 | B |
| ATOM | 4361 | CG | ARG | B | 98 | 23.014 | −19.351 | 33.463 | 1.00 | 199.41 | B |
| ATOM | 4362 | CD | ARG | B | 98 | 22.889 | −19.850 | 34.893 | 1.00 | 199.41 | B |
| ATOM | 4363 | NE | ARG | B | 98 | 24.047 | −20.651 | 35.284 | 1.00 | 199.41 | B |
| ATOM | 4364 | CZ | ARG | B | 98 | 24.464 | −21.735 | 34.637 | 1.00 | 199.41 | B |
| ATOM | 4365 | NH1 | ARG | B | 98 | 25.529 | −22.396 | 35.070 | 1.00 | 199.41 | B |
| ATOM | 4366 | NH2 | ARG | B | 98 | 23.820 | −22.161 | 33.558 | 1.00 | 199.41 | B |
| ATOM | 4367 | C | ARG | B | 98 | 23.409 | −16.454 | 33.935 | 1.00 | 200.81 | B |
| ATOM | 4368 | O | ARG | B | 98 | 24.351 | −17.136 | 34.342 | 1.00 | 200.81 | B |
| ATOM | 4369 | N | GLU | B | 99 | 23.559 | −15.199 | 33.516 | 1.00 | 193.03 | B |
| ATOM | 4370 | CA | GLU | B | 99 | 24.865 | −14.539 | 33.492 | 1.00 | 193.03 | B |
| ATOM | 4371 | CB | GLU | B | 99 | 24.703 | −13.017 | 33.363 | 1.00 | 165.68 | B |
| ATOM | 4372 | CG | GLU | B | 99 | 23.269 | −12.510 | 33.469 | 1.00 | 165.68 | B |
| ATOM | 4373 | CD | GLU | B | 99 | 22.534 | −12.553 | 32.143 | 1.00 | 165.68 | B |
| ATOM | 4374 | OE1 | GLU | B | 99 | 22.656 | −13.571 | 31.430 | 1.00 | 165.68 | B |
| ATOM | 4375 | OE2 | GLU | B | 99 | 21.829 | −11.572 | 31.818 | 1.00 | 165.68 | B |
| ATOM | 4376 | C | GLU | B | 99 | 25.690 | −14.849 | 34.740 | 1.00 | 193.03 | B |
| ATOM | 4377 | O | GLU | B | 99 | 26.850 | −15.245 | 34.636 | 1.00 | 193.03 | B |
| ATOM | 4378 | N | THR | B | 100 | 25.082 | −14.666 | 35.910 | 1.00 | 200.84 | B |
| ATOM | 4379 | CA | THR | B | 100 | 25.736 | −14.912 | 37.195 | 1.00 | 200.84 | B |
| ATOM | 4380 | CB | THR | B | 100 | 25.852 | −16.429 | 37.488 | 1.00 | 166.34 | B |
| ATOM | 4381 | OG1 | THR | B | 100 | 26.536 | −17.077 | 36.410 | 1.00 | 166.62 | B |
| ATOM | 4382 | CG2 | THR | B | 100 | 24.473 | −17.051 | 37.649 | 1.00 | 167.51 | B |
| ATOM | 4383 | C | THR | B | 100 | 27.128 | −14.278 | 37.275 | 1.00 | 200.84 | B |
| ATOM | 4384 | O | THR | B | 100 | 28.041 | −14.657 | 36.541 | 1.00 | 200.84 | B |
| ATOM | 4385 | N | PRO | B | 101 | 27.304 | −13.301 | 38.179 | 1.00 | 200.84 | B |
| ATOM | 4386 | CD | PRO | B | 101 | 26.311 | −12.832 | 39.164 | 1.00 | 106.21 | B |
| ATOM | 4387 | CA | PRO | B | 101 | 28.579 | −12.604 | 38.364 | 1.00 | 200.84 | B |
| ATOM | 4388 | CB | PRO | B | 101 | 28.395 | −11.915 | 39.712 | 1.00 | 107.16 | B |
| ATOM | 4389 | CG | PRO | B | 101 | 26.944 | −11.559 | 39.686 | 1.00 | 105.19 | B |
| ATOM | 4390 | C | PRO | B | 101 | 29.791 | −13.533 | 38.342 | 1.00 | 200.84 | B |
| ATOM | 4391 | O | PRO | B | 101 | 29.985 | −14.342 | 39.250 | 1.00 | 200.84 | B |
| ATOM | 4392 | N | GLU | B | 102 | 30.596 | −13.405 | 37.292 | 1.00 | 200.84 | B |
| ATOM | 4393 | CA | GLU | B | 102 | 31.800 | −14.211 | 37.117 | 1.00 | 200.84 | B |
| ATOM | 4394 | CB | GLU | B | 102 | 32.644 | −13.618 | 35.979 | 1.00 | 123.28 | B |
| ATOM | 4395 | CG | GLU | B | 102 | 33.982 | −14.304 | 35.728 | 1.00 | 124.48 | B |
| ATOM | 4396 | CD | GLU | B | 102 | 35.020 | −13.991 | 36.795 | 1.00 | 125.79 | B |
| ATOM | 4397 | OE1 | GLU | B | 102 | 35.347 | −12.798 | 36.987 | 1.00 | 126.60 | B |
| ATOM | 4398 | OE2 | GLU | B | 102 | 35.509 | −14.941 | 37.440 | 1.00 | 124.91 | B |
| ATOM | 4399 | C | GLU | B | 102 | 32.624 | −14.303 | 38.406 | 1.00 | 200.84 | B |
| ATOM | 4400 | O | GLU | B | 102 | 33.169 | −15.360 | 38.728 | 1.00 | 200.12 | B |
| ATOM | 4401 | N | GLY | B | 103 | 32.706 | −13.197 | 39.140 | 1.00 | 173.10 | B |
| ATOM | 4402 | CA | GLY | B | 103 | 33.466 | −13.174 | 40.379 | 1.00 | 170.42 | B |
| ATOM | 4403 | C | GLY | B | 103 | 34.053 | −11.801 | 40.649 | 1.00 | 168.29 | B |
| ATOM | 4404 | O | GLY | B | 103 | 34.547 | −11.527 | 41.744 | 1.00 | 162.78 | B |
| ATOM | 4405 | N | ALA | B | 104 | 33.997 | −10.935 | 39.640 | 1.00 | 187.49 | B |
| ATOM | 4406 | CA | ALA | B | 104 | 34.516 | −9.577 | 39.746 | 1.00 | 187.49 | B |
| ATOM | 4407 | CB | ALA | B | 104 | 35.877 | −9.485 | 39.058 | 1.00 | 96.77 | B |
| ATOM | 4408 | C | ALA | B | 104 | 33.537 | −8.592 | 39.110 | 1.00 | 187.49 | B |
| ATOM | 4409 | O | ALA | B | 104 | 33.300 | −8.645 | 37.903 | 1.00 | 187.49 | B |
| ATOM | 4410 | N | GLU | B | 105 | 32.975 | −7.708 | 39.937 | 1.00 | 181.54 | B |
| ATOM | 4411 | CA | GLU | B | 105 | 32.014 | −6.678 | 39.520 | 1.00 | 181.54 | B |
| ATOM | 4412 | CB | GLU | B | 105 | 32.177 | −6.332 | 38.033 | 1.00 | 199.72 | B |
| ATOM | 4413 | CG | GLU | B | 105 | 33.481 | −5.635 | 37.689 | 1.00 | 199.72 | B |
| ATOM | 4414 | CD | GLU | B | 105 | 33.639 | −4.316 | 38.415 | 1.00 | 199.72 | B |
| ATOM | 4415 | OE1 | GLU | B | 105 | 33.727 | −4.330 | 39.661 | 1.00 | 199.72 | B |
| ATOM | 4416 | OE2 | GLU | B | 105 | 33.671 | −3.264 | 37.741 | 1.00 | 198.49 | B |
| ATOM | 4417 | C | GLU | B | 105 | 30.553 | −7.036 | 39.793 | 1.00 | 181.54 | B |
| ATOM | 4418 | O | GLU | B | 105 | 30.171 | −7.271 | 40.940 | 1.00 | 181.54 | B |
| ATOM | 4419 | N | ALA | B | 106 | 29.749 | −7.057 | 38.729 | 1.00 | 162.64 | B |
| ATOM | 4420 | CA | ALA | B | 106 | 28.318 | −7.368 | 38.785 | 1.00 | 162.64 | B |
| ATOM | 4421 | CB | ALA | B | 106 | 28.037 | −8.443 | 39.844 | 1.00 | 45.35 | B |
| ATOM | 4422 | C | ALA | B | 106 | 27.474 | −6.119 | 39.056 | 1.00 | 162.64 | B |
| ATOM | 4423 | O | ALA | B | 106 | 27.079 | −5.853 | 40.191 | 1.00 | 162.64 | B |
| ATOM | 4424 | N | LYS | B | 107 | 27.202 | −5.359 | 37.997 | 1.00 | 138.43 | B |
| ATOM | 4425 | CA | LYS | B | 107 | 26.405 | −4.135 | 38.089 | 1.00 | 132.55 | B |
| ATOM | 4426 | CB | LYS | B | 107 | 26.462 | −3.361 | 36.765 | 1.00 | 190.21 | B |
| ATOM | 4427 | CG | LYS | B | 107 | 27.755 | −2.601 | 36.497 | 1.00 | 190.16 | B |
| ATOM | 4428 | CD | LYS | B | 107 | 28.949 | −3.524 | 36.334 | 1.00 | 191.59 | B |
| ATOM | 4429 | CE | LYS | B | 107 | 30.173 | −2.745 | 35.874 | 1.00 | 187.63 | B |
| ATOM | 4430 | NZ | LYS | B | 107 | 30.490 | −1.601 | 36.774 | 1.00 | 187.89 | B |
| ATOM | 4431 | C | LYS | B | 107 | 24.943 | −4.435 | 38.420 | 1.00 | 128.03 | B |
| ATOM | 4432 | O | LYS | B | 107 | 24.511 | −5.585 | 38.368 | 1.00 | 127.16 | B |
| ATOM | 4433 | N | PRO | B | 108 | 24.163 | −3.397 | 38.770 | 1.00 | 98.29 | B |
| ATOM | 4434 | CD | PRO | B | 108 | 24.581 | −2.013 | 39.066 | 1.00 | 75.18 | B |
| ATOM | 4435 | CA | PRO | B | 108 | 22.749 | −3.585 | 39.099 | 1.00 | 97.03 | B |
| ATOM | 4436 | CB | PRO | B | 108 | 22.441 | −2.363 | 39.965 | 1.00 | 74.33 | B |
| ATOM | 4437 | CG | PRO | B | 108 | 23.254 | −1.307 | 39.316 | 1.00 | 71.71 | B |
| ATOM | 4438 | C | PRO | B | 108 | 21.899 | −3.631 | 37.828 | 1.00 | 96.26 | B |
| ATOM | 4439 | O | PRO | B | 108 | 22.330 | −3.165 | 36.773 | 1.00 | 94.44 | B |
| ATOM | 4440 | N | TRP | B | 109 | 20.703 | −4.208 | 37.928 | 1.00 | 66.80 | B |
| ATOM | 4441 | CA | TRP | B | 109 | 19.804 | −4.285 | 36.783 | 1.00 | 64.25 | B |
| ATOM | 4442 | CB | TRP | B | 109 | 19.728 | −5.710 | 36.230 | 1.00 | 77.73 | B |
| ATOM | 4443 | CG | TRP | B | 109 | 19.348 | −6.743 | 37.239 | 1.00 | 77.42 | B |
| ATOM | 4444 | CD2 | TRP | B | 109 | 18.025 | −7.092 | 37.663 | 1.00 | 76.43 | B |
| ATOM | 4445 | CE2 | TRP | B | 109 | 18.150 | −8.134 | 38.606 | 1.00 | 75.68 | B |
| ATOM | 4446 | CE3 | TRP | B | 109 | 16.746 | −6.627 | 37.338 | 1.00 | 76.53 | B |
| ATOM | 4447 | CD1 | TRP | B | 109 | 20.192 | −7.556 | 37.925 | 1.00 | 74.52 | B |
| ATOM | 4448 | NE1 | TRP | B | 109 | 19.484 | −8.398 | 38.747 | 1.00 | 72.84 | B |
| ATOM | 4449 | CZ2 | TRP | B | 109 | 17.047 | −8.722 | 39.229 | 1.00 | 72.13 | B |
| ATOM | 4450 | CZ3 | TRP | B | 109 | 15.643 | −7.212 | 37.961 | 1.00 | 76.29 | B |
| ATOM | 4451 | CH2 | TRP | B | 109 | 15.805 | −8.250 | 38.896 | 1.00 | 71.93 | B |
| ATOM | 4452 | C | TRP | B | 109 | 18.402 | −3.817 | 37.132 | 1.00 | 65.86 | B |
| ATOM | 4453 | O | TRP | B | 109 | 17.922 | −4.023 | 38.248 | 1.00 | 67.47 | B |
| ATOM | 4454 | N | TYR | B | 110 | 17.757 | −3.184 | 36.159 | 1.00 | 68.59 | B |
| ATOM | 4455 | CA | TYR | B | 110 | 16.404 | −2.687 | 36.327 | 1.00 | 67.23 | B |
| ATOM | 4456 | CB | TYR | B | 110 | 16.372 | −1.165 | 36.209 | 1.00 | 69.62 | B |
| ATOM | 4457 | CG | TYR | B | 110 | 17.153 | −0.438 | 37.276 | 1.00 | 73.17 | B |
| ATOM | 4458 | CD1 | TYR | B | 110 | 18.548 | −0.429 | 37.273 | 1.00 | 77.26 | B |
| ATOM | 4459 | CE1 | TYR | B | 110 | 19.263 | 0.285 | 38.234 | 1.00 | 77.58 | B |
| ATOM | 4460 | CD2 | TYR | B | 110 | 16.494 | 0.275 | 38.268 | 1.00 | 73.40 | B |
| ATOM | 4461 | CE2 | TYR | B | 110 | 17.197 | 0.988 | 39.233 | 1.00 | 78.77 | B |
| ATOM | 4462 | CZ | TYR | B | 110 | 18.575 | 0.993 | 39.211 | 1.00 | 79.06 | B |
| ATOM | 4463 | OH | TYR | B | 110 | 19.247 | 1.723 | 40.163 | 1.00 | 80.26 | B |
| ATOM | 4464 | C | TYR | B | 110 | 15.518 | −3.296 | 35.256 | 1.00 | 65.74 | B |
| ATOM | 4465 | O | TYR | B | 110 | 15.916 | −3.402 | 34.098 | 1.00 | 66.93 | B |
| ATOM | 4466 | N | GLU | B | 111 | 14.313 | −3.695 | 35.649 | 1.00 | 62.77 | B |
| ATOM | 4467 | CA | GLU | B | 111 | 13.366 | −4.298 | 34.721 | 1.00 | 60.79 | B |
| ATOM | 4468 | CB | GLU | B | 111 | 13.503 | −5.818 | 34.771 | 1.00 | 76.65 | B |
| ATOM | 4469 | CG | GLU | B | 111 | 13.431 | −6.482 | 33.426 | 1.00 | 83.57 | B |
| ATOM | 4470 | CD | GLU | B | 111 | 14.534 | −6.020 | 32.505 | 1.00 | 88.54 | B |
| ATOM | 4471 | OE1 | GLU | B | 111 | 15.712 | −6.209 | 32.866 | 1.00 | 88.75 | B |
| ATOM | 4472 | OE2 | GLU | B | 111 | 14.226 | −5.466 | 31.424 | 1.00 | 95.70 | B |
| ATOM | 4473 | C | GLU | B | 111 | 11.934 | −3.892 | 35.080 | 1.00 | 61.37 | B |
| ATOM | 4474 | O | GLU | B | 111 | 11.399 | −4.309 | 36.110 | 1.00 | 66.73 | B |
| ATOM | 4475 | N | PRO | B | 112 | 11.305 | −3.042 | 34.250 | 1.00 | 53.83 | B |
| ATOM | 4476 | CD | PRO | B | 112 | 11.940 | −2.192 | 33.226 | 1.00 | 40.62 | B |
| ATOM | 4477 | CA | PRO | B | 112 | 9.930 | −2.595 | 34.494 | 1.00 | 53.06 | B |
| ATOM | 4478 | CB | PRO | B | 112 | 9.950 | −1.168 | 33.978 | 1.00 | 36.69 | B |
| ATOM | 4479 | CG | PRO | B | 112 | 10.780 | −1.303 | 32.764 | 1.00 | 37.82 | B |
| ATOM | 4480 | C | PRO | B | 112 | 8.917 | −3.450 | 33.727 | 1.00 | 50.09 | B |
| ATOM | 4481 | O | PRO | B | 112 | 9.254 | −4.076 | 32.717 | 1.00 | 50.22 | B |
| ATOM | 4482 | N | ILE | B | 113 | 7.684 | −3.488 | 34.216 | 1.00 | 41.31 | B |
| ATOM | 4483 | CA | ILE | B | 113 | 6.623 | −4.238 | 33.545 | 1.00 | 43.28 | B |
| ATOM | 4484 | CB | ILE | B | 113 | 6.375 | −5.611 | 34.184 | 1.00 | 53.81 | B |
| ATOM | 4485 | CG2 | ILE | B | 113 | 5.246 | −6.318 | 33.453 | 1.00 | 49.83 | B |
| ATOM | 4486 | CG1 | ILE | B | 113 | 7.635 | −6.462 | 34.115 | 1.00 | 50.87 | B |
| ATOM | 4487 | CD1 | ILE | B | 113 | 7.420 | −7.882 | 34.616 | 1.00 | 51.35 | B |
| ATOM | 4488 | C | ILE | B | 113 | 5.320 | −3.459 | 33.635 | 1.00 | 47.50 | B |
| ATOM | 4489 | O | ILE | B | 113 | 4.910 | −3.058 | 34.727 | 1.00 | 50.90 | B |
| ATOM | 4490 | N | TYR | B | 114 | 4.676 | −3.235 | 32.493 | 1.00 | 36.63 | B |
| ATOM | 4491 | CA | TYR | B | 114 | 3.402 | −2.519 | 32.486 | 1.00 | 34.91 | B |
| ATOM | 4492 | CB | TYR | B | 114 | 3.454 | −1.329 | 31.534 | 1.00 | 39.03 | B |
| ATOM | 4493 | CG | TYR | B | 114 | 2.251 | −0.421 | 31.640 | 1.00 | 36.89 | B |
| ATOM | 4494 | CD1 | TYR | B | 114 | 2.198 | 0.582 | 32.605 | 1.00 | 41.18 | B |
| ATOM | 4495 | CE1 | TYR | B | 114 | 1.086 | 1.418 | 32.716 | 1.00 | 40.75 | B |
| ATOM | 4496 | CD2 | TYR | B | 114 | 1.154 | −0.571 | 30.779 | 1.00 | 37.06 | B |
| ATOM | 4497 | CE2 | TYR | B | 114 | 0.040 | 0.257 | 30.884 | 1.00 | 41.39 | B |
| ATOM | 4498 | CZ | TYR | B | 114 | 0.019 | 1.253 | 31.859 | 1.00 | 41.47 | B |
| ATOM | 4499 | OH | TYR | B | 114 | −1.061 | 2.096 | 31.983 | 1.00 | 40.97 | B |
| ATOM | 4500 | C | TYR | B | 114 | 2.276 | −3.460 | 32.059 | 1.00 | 34.75 | B |
| ATOM | 4501 | O | TYR | B | 114 | 2.460 | −4.320 | 31.206 | 1.00 | 37.66 | B |
| ATOM | 4502 | N | LEU | B | 115 | 1.114 | −3.284 | 32.666 | 1.00 | 37.95 | B |
| ATOM | 4503 | CA | LEU | B | 115 | −0.057 | −4.100 | 32.374 | 1.00 | 36.99 | B |
| ATOM | 4504 | CB | LEU | B | 115 | −0.258 | −5.115 | 33.496 | 1.00 | 50.09 | B |
| ATOM | 4505 | CG | LEU | B | 115 | 0.013 | −6.603 | 33.275 | 1.00 | 53.34 | B |
| ATOM | 4506 | CD1 | LEU | B | 115 | 1.210 | −6.821 | 32.371 | 1.00 | 51.72 | B |
| ATOM | 4507 | CD2 | LEU | B | 115 | 0.225 | −7.251 | 34.644 | 1.00 | 56.64 | B |
| ATOM | 4508 | C | LEU | B | 115 | −1.286 | −3.192 | 32.289 | 1.00 | 39.43 | B |
| ATOM | 4509 | O | LEU | B | 115 | −1.431 | −2.252 | 33.087 | 1.00 | 41.96 | B |
| ATOM | 4510 | N | GLY | B | 116 | −2.168 | −3.457 | 31.328 | 1.00 | 35.21 | B |
| ATOM | 4511 | CA | GLY | B | 116 | −3.356 | −2.629 | 31.221 | 1.00 | 29.55 | B |
| ATOM | 4512 | C | GLY | B | 116 | −4.337 | −3.085 | 30.175 | 1.00 | 33.40 | B |
| ATOM | 4513 | O | GLY | B | 116 | −3.931 | −3.534 | 29.117 | 1.00 | 32.28 | B |
| ATOM | 4514 | N | GLY | B | 117 | −5.628 | −2.948 | 30.475 | 1.00 | 31.11 | B |
| ATOM | 4515 | CA | GLY | B | 117 | −6.681 | −3.362 | 29.558 | 1.00 | 34.11 | B |
| ATOM | 4516 | C | GLY | B | 117 | −8.042 | −2.977 | 30.103 | 1.00 | 35.42 | B |
| ATOM | 4517 | O | GLY | B | 117 | −8.158 | −2.539 | 31.259 | 1.00 | 36.80 | B |
| ATOM | 4518 | N | VAL | B | 118 | −9.076 | −3.132 | 29.277 | 1.00 | 38.74 | B |
| ATOM | 4519 | CA | VAL | B | 118 | −10.449 | −2.786 | 29.670 | 1.00 | 41.52 | B |
| ATOM | 4520 | CB | VAL | B | 118 | −11.138 | −1.979 | 28.562 | 1.00 | 29.39 | B |
| ATOM | 4521 | CG1 | VAL | B | 118 | −12.576 | −1.677 | 28.937 | 1.00 | 30.79 | B |
| ATOM | 4522 | CG2 | VAL | B | 118 | −10.371 | −0.692 | 28.341 | 1.00 | 29.45 | B |
| ATOM | 4523 | C | VAL | B | 118 | −11.248 | −4.047 | 29.974 | 1.00 | 39.43 | B |
| ATOM | 4524 | O | VAL | B | 118 | −11.160 | −5.024 | 29.238 | 1.00 | 43.38 | B |
| ATOM | 4525 | N | PHE | B | 119 | −12.015 | −4.017 | 31.068 | 1.00 | 38.53 | B |
| ATOM | 4526 | CA | PHE | B | 119 | −12.798 | −5.178 | 31.496 | 1.00 | 39.84 | B |
| ATOM | 4527 | CB | PHE | B | 119 | −12.015 | −5.950 | 32.566 | 1.00 | 38.55 | B |
| ATOM | 4528 | CG | PHE | B | 119 | −10.701 | −6.494 | 32.087 | 1.00 | 36.15 | B |
| ATOM | 4529 | CD1 | PHE | B | 119 | −9.560 | −5.701 | 32.087 | 1.00 | 34.34 | B |
| ATOM | 4530 | CD2 | PHE | B | 119 | −10.616 | −7.792 | 31.575 | 1.00 | 34.83 | B |
| ATOM | 4531 | CE1 | PHE | B | 119 | −8.341 | −6.193 | 31.571 | 1.00 | 35.41 | B |
| ATOM | 4532 | CE2 | PHE | B | 119 | −9.411 | −8.290 | 31.060 | 1.00 | 35.64 | B |
| ATOM | 4533 | CZ | PHE | B | 119 | −8.273 | −7.489 | 31.056 | 1.00 | 34.73 | B |
| ATOM | 4534 | C | PHE | B | 119 | −14.197 | −4.880 | 32.046 | 1.00 | 40.75 | B |
| ATOM | 4535 | O | PHE | B | 119 | −14.442 | −3.820 | 32.620 | 1.00 | 36.85 | B |
| ATOM | 4536 | N | GLN | B | 120 | −15.107 | −5.836 | 31.868 | 1.00 | 42.95 | B |
| ATOM | 4537 | CA | GLN | B | 120 | −16.480 | −5.738 | 32.374 | 1.00 | 46.32 | B |
| ATOM | 4538 | CB | GLN | B | 120 | −17.401 | −6.655 | 31.586 | 1.00 | 61.74 | B |
| ATOM | 4539 | CG | GLN | B | 120 | −18.556 | −5.974 | 30.894 | 1.00 | 61.89 | B |
| ATOM | 4540 | CD | GLN | B | 120 | −19.397 | −5.148 | 31.830 | 1.00 | 61.49 | B |
| ATOM | 4541 | OE1 | GLN | B | 120 | −19.683 | −5.564 | 32.946 | 1.00 | 61.05 | B |
| ATOM | 4542 | NE2 | GLN | B | 120 | −19.818 | −3.969 | 31.373 | 1.00 | 64.32 | B |
| ATOM | 4543 | C | GLN | B | 120 | −16.407 | −6.251 | 33.806 | 1.00 | 45.46 | B |
| ATOM | 4544 | O | GLN | B | 120 | −15.990 | −7.382 | 34.013 | 1.00 | 48.16 | B |
| ATOM | 4545 | N | LEU | B | 121 | −16.805 | −5.439 | 34.784 | 1.00 | 36.79 | B |
| ATOM | 4546 | CA | LEU | B | 121 | −16.745 | −5.829 | 36.190 | 1.00 | 38.45 | B |
| ATOM | 4547 | CB | LEU | B | 121 | −15.736 | −4.948 | 36.930 | 1.00 | 39.59 | B |
| ATOM | 4548 | CG | LEU | B | 121 | −14.286 | −5.417 | 37.055 | 1.00 | 37.86 | B |
| ATOM | 4549 | CD1 | LEU | B | 121 | −13.885 | −6.279 | 35.881 | 1.00 | 39.25 | B |
| ATOM | 4550 | CD2 | LEU | B | 121 | −13.390 | −4.184 | 37.182 | 1.00 | 33.35 | B |
| ATOM | 4551 | C | LEU | B | 121 | −18.090 | −5.723 | 36.891 | 1.00 | 40.49 | B |
| ATOM | 4552 | O | LEU | B | 121 | −18.920 | −4.880 | 36.525 | 1.00 | 45.76 | B |
| ATOM | 4553 | N | GLU | B | 122 | −18.292 | −6.577 | 37.901 | 1.00 | 43.64 | B |
| ATOM | 4554 | CA | GLU | B | 122 | −19.524 | −6.604 | 38.698 | 1.00 | 45.18 | B |
| ATOM | 4555 | CB | GLU | B | 122 | −19.888 | −8.022 | 39.136 | 1.00 | 96.93 | B |
| ATOM | 4556 | CG | GLU | B | 122 | −20.142 | −9.051 | 38.067 | 1.00 | 99.97 | B |
| ATOM | 4557 | CD | GLU | B | 122 | −20.369 | −10.438 | 38.678 | 1.00 | 105.25 | B |
| ATOM | 4558 | OE1 | GLU | B | 122 | −19.435 | −10.963 | 39.331 | 1.00 | 106.75 | B |
| ATOM | 4559 | OE2 | GLU | B | 122 | −21.480 | −10.996 | 38.517 | 1.00 | 105.34 | B |
| ATOM | 4560 | C | GLU | B | 122 | −19.317 | −5.816 | 39.984 | 1.00 | 43.98 | B |
| ATOM | 4561 | O | GLU | B | 122 | −18.201 | −5.738 | 40.489 | 1.00 | 45.17 | B |
| ATOM | 4562 | N | LYS | B | 123 | −20.392 | −5.252 | 40.525 | 1.00 | 45.69 | B |
| ATOM | 4563 | CA | LYS | B | 123 | −20.288 | −4.530 | 41.781 | 1.00 | 46.54 | B |
| ATOM | 4564 | CB | LYS | B | 123 | −21.647 | −3.982 | 42.208 | 1.00 | 62.86 | B |
| ATOM | 4565 | CG | LYS | B | 123 | −21.653 | −3.421 | 43.607 | 1.00 | 59.02 | B |
| ATOM | 4566 | CD | LYS | B | 123 | −23.036 | −2.985 | 44.015 | 1.00 | 56.69 | B |
| ATOM | 4567 | CE | LYS | B | 123 | −23.038 | −2.497 | 45.453 | 1.00 | 58.42 | B |
| ATOM | 4568 | NZ | LYS | B | 123 | −24.406 | −2.122 | 45.920 | 1.00 | 62.43 | B |
| ATOM | 4569 | C | LYS | B | 123 | −19.813 | −5.554 | 42.798 | 1.00 | 47.36 | B |
| ATOM | 4570 | O | LYS | B | 123 | −20.403 | −6.625 | 42.933 | 1.00 | 52.12 | B |
| ATOM | 4571 | N | GLY | B | 124 | −18.731 | −5.238 | 43.495 | 1.00 | 41.61 | B |
| ATOM | 4572 | CA | GLY | B | 124 | −18.208 | −6.154 | 44.486 | 1.00 | 42.72 | B |
| ATOM | 4573 | C | GLY | B | 124 | −16.964 | −6.926 | 44.080 | 1.00 | 44.13 | B |
| ATOM | 4574 | O | GLY | B | 124 | −16.357 | −7.591 | 44.925 | 1.00 | 44.93 | B |
| ATOM | 4575 | N | ASP | B | 125 | −16.578 | −6.864 | 42.807 | 1.00 | 42.70 | B |
| ATOM | 4576 | CA | ASP | B | 125 | −15.387 | −7.579 | 42.358 | 1.00 | 41.49 | B |
| ATOM | 4577 | CB | ASP | B | 125 | −15.226 | −7.453 | 40.847 | 1.00 | 61.29 | B |
| ATOM | 4578 | CG | ASP | B | 125 | −16.254 | −8.254 | 40.075 | 1.00 | 66.75 | B |
| ATOM | 4579 | OD1 | ASP | B | 125 | −16.970 | −9.072 | 40.688 | 1.00 | 68.40 | B |
| ATOM | 4580 | OD2 | ASP | B | 125 | −16.334 | −8.072 | 38.844 | 1.00 | 65.41 | B |
| ATOM | 4581 | C | ASP | B | 125 | −14.119 | −7.062 | 43.045 | 1.00 | 40.54 | B |
| ATOM | 4582 | O | ASP | B | 125 | −14.022 | −5.876 | 43.386 | 1.00 | 37.45 | B |
| ATOM | 4583 | N | ARG | B | 126 | −13.155 | −7.956 | 43.248 | 1.00 | 51.79 | B |
| ATOM | 4584 | CA | ARG | B | 126 | −11.897 | −7.613 | 43.895 | 1.00 | 47.27 | B |
| ATOM | 4585 | CB | ARG | B | 126 | −11.636 | −8.552 | 45.071 | 1.00 | 82.06 | B |
| ATOM | 4586 | CG | ARG | B | 126 | −12.551 | −8.379 | 46.266 | 1.00 | 80.55 | B |
| ATOM | 4587 | CD | ARG | B | 126 | −12.349 | −9.522 | 47.256 | 1.00 | 85.47 | B |
| ATOM | 4588 | NE | ARG | B | 126 | −12.931 | −9.214 | 48.556 | 1.00 | 89.02 | B |
| ATOM | 4589 | CZ | ARG | B | 126 | −12.370 | −8.410 | 49.462 | 1.00 | 92.25 | B |
| ATOM | 4590 | NH1 | ARG | B | 126 | −12.979 | −8.180 | 50.625 | 1.00 | 91.92 | B |
| ATOM | 4591 | NH2 | ARG | B | 126 | −11.189 | −7.850 | 49.221 | 1.00 | 90.44 | B |
| ATOM | 4592 | C | ARG | B | 126 | −10.755 | −7.746 | 42.900 | 1.00 | 48.11 | B |
| ATOM | 4593 | O | ARG | B | 126 | −10.578 | −8.798 | 42.297 | 1.00 | 45.33 | B |
| ATOM | 4594 | N | LEU | B | 127 | −9.967 | −6.692 | 42.732 | 1.00 | 38.62 | B |
| ATOM | 4595 | CA | LEU | B | 127 | −8.844 | −6.729 | 41.800 | 1.00 | 35.00 | B |
| ATOM | 4596 | CB | LEU | B | 127 | −8.891 | −5.509 | 40.887 | 1.00 | 34.87 | B |
| ATOM | 4597 | CG | LEU | B | 127 | −10.172 | −5.431 | 40.052 | 1.00 | 33.85 | B |
| ATOM | 4598 | CD1 | LEU | B | 127 | −10.291 | −4.070 | 39.393 | 1.00 | 34.43 | B |
| ATOM | 4599 | CD2 | LEU | B | 127 | −10.153 | −6.525 | 38.999 | 1.00 | 34.51 | B |
| ATOM | 4600 | C | LEU | B | 127 | −7.535 | −6.766 | 42.563 | 1.00 | 34.75 | B |
| ATOM | 4601 | O | LEU | B | 127 | −7.367 | −6.040 | 43.536 | 1.00 | 30.95 | B |
| ATOM | 4602 | N | SER | B | 128 | −6.611 | −7.618 | 42.120 | 1.00 | 43.34 | B |
| ATOM | 4603 | CA | SER | B | 128 | −5.306 | −7.771 | 42.772 | 1.00 | 43.01 | B |
| ATOM | 4604 | CB | SER | B | 128 | −5.140 | −9.182 | 43.332 | 1.00 | 73.11 | B |
| ATOM | 4605 | OG | SER | B | 128 | −6.335 | −9.650 | 43.924 | 1.00 | 78.96 | B |
| ATOM | 4606 | C | SER | B | 128 | −4.179 | −7.529 | 41.789 | 1.00 | 42.19 | B |
| ATOM | 4607 | O | SER | B | 128 | −4.252 | −7.941 | 40.639 | 1.00 | 43.47 | B |
| ATOM | 4608 | N | ALA | B | 129 | −3.138 | −6.851 | 42.252 | 1.00 | 50.95 | B |
| ATOM | 4609 | CA | ALA | B | 129 | −1.975 | −6.572 | 41.431 | 1.00 | 51.92 | B |
| ATOM | 4610 | CB | ALA | B | 129 | −1.812 | −5.067 | 41.249 | 1.00 | 26.61 | B |
| ATOM | 4611 | C | ALA | B | 129 | −0.816 | −7.160 | 42.228 | 1.00 | 50.69 | B |
| ATOM | 4612 | O | ALA | B | 129 | −0.322 | −6.545 | 43.167 | 1.00 | 51.57 | B |
| ATOM | 4613 | N | GLU | B | 130 | −0.390 | −8.362 | 41.860 | 1.00 | 43.43 | B |
| ATOM | 4614 | CA | GLU | B | 130 | 0.672 | −9.039 | 42.588 | 1.00 | 44.40 | B |
| ATOM | 4615 | CB | GLU | B | 130 | 0.107 | −10.328 | 43.189 | 1.00 | 89.61 | B |
| ATOM | 4616 | CG | GLU | B | 130 | −1.059 | −10.083 | 44.132 | 1.00 | 89.15 | B |
| ATOM | 4617 | CD | GLU | B | 130 | −1.948 | −11.301 | 44.326 | 1.00 | 94.64 | B |
| ATOM | 4618 | OE1 | GLU | B | 130 | −2.903 | −11.212 | 45.133 | 1.00 | 96.01 | B |
| ATOM | 4619 | OE2 | GLU | B | 130 | −1.704 | −12.338 | 43.674 | 1.00 | 94.45 | B |
| ATOM | 4620 | C | GLU | B | 130 | 1.927 | −9.352 | 41.777 | 1.00 | 40.79 | B |
| ATOM | 4621 | O | GLU | B | 130 | 1.919 | −9.317 | 40.540 | 1.00 | 40.71 | B |
| ATOM | 4622 | N | ILE | B | 131 | 3.015 | −9.637 | 42.490 | 1.00 | 49.62 | B |
| ATOM | 4623 | CA | ILE | B | 131 | 4.286 | −9.995 | 41.866 | 1.00 | 52.16 | B |
| ATOM | 4624 | CB | ILE | B | 131 | 5.341 | −8.879 | 41.996 | 1.00 | 53.36 | B |
| ATOM | 4625 | CG2 | ILE | B | 131 | 4.920 | −7.674 | 41.186 | 1.00 | 53.59 | B |
| ATOM | 4626 | CG1 | ILE | B | 131 | 5.538 | −8.525 | 43.468 | 1.00 | 49.55 | B |
| ATOM | 4627 | CD1 | ILE | B | 131 | 6.610 | −7.491 | 43.703 | 1.00 | 48.16 | B |
| ATOM | 4628 | C | ILE | B | 131 | 4.792 | −11.218 | 42.604 | 1.00 | 54.05 | B |
| ATOM | 4629 | O | ILE | B | 131 | 4.335 | −11.508 | 43.707 | 1.00 | 54.77 | B |
| ATOM | 4630 | N | ASN | B | 132 | 5.744 | −11.926 | 42.011 | 1.00 | 68.42 | B |
| ATOM | 4631 | CA | ASN | B | 132 | 6.284 | −13.124 | 42.641 | 1.00 | 65.42 | B |
| ATOM | 4632 | CB | ASN | B | 132 | 6.543 | −14.169 | 41.571 | 1.00 | 52.92 | B |
| ATOM | 4633 | CG | ASN | B | 132 | 7.632 | −13.751 | 40.622 | 1.00 | 51.62 | B |
| ATOM | 4634 | OD1 | ASN | B | 132 | 7.694 | −12.597 | 40.203 | 1.00 | 53.31 | B |
| ATOM | 4635 | ND2 | ASN | B | 132 | 8.503 | −14.687 | 40.272 | 1.00 | 49.45 | B |
| ATOM | 4636 | C | ASN | B | 132 | 7.569 | −12.890 | 43.454 | 1.00 | 63.07 | B |
| ATOM | 4637 | O | ASN | B | 132 | 7.842 | −13.627 | 44.398 | 1.00 | 66.24 | B |
| ATOM | 4638 | N | ARG | B | 133 | 8.351 | −11.870 | 43.097 | 1.00 | 64.67 | B |
| ATOM | 4639 | CA | ARG | B | 133 | 9.599 | −11.580 | 43.804 | 1.00 | 60.26 | B |
| ATOM | 4640 | CB | ARG | B | 133 | 10.785 | −11.727 | 42.846 | 1.00 | 67.24 | B |
| ATOM | 4641 | CG | ARG | B | 133 | 10.955 | −13.124 | 42.282 | 1.00 | 69.82 | B |
| ATOM | 4642 | CD | ARG | B | 133 | 11.108 | −14.145 | 43.393 | 1.00 | 75.06 | B |
| ATOM | 4643 | NE | ARG | B | 133 | 12.257 | −13.874 | 44.254 | 1.00 | 72.92 | B |
| ATOM | 4644 | CZ | ARG | B | 133 | 13.531 | −14.037 | 43.893 | 1.00 | 74.02 | B |
| ATOM | 4645 | NH1 | ARG | B | 133 | 14.508 | −13.763 | 44.752 | 1.00 | 76.70 | B |
| ATOM | 4646 | NH2 | ARG | B | 133 | 13.836 | −14.478 | 42.678 | 1.00 | 73.31 | B |
| ATOM | 4647 | C | ARG | B | 133 | 9.632 | −10.194 | 44.444 | 1.00 | 64.85 | B |
| ATOM | 4648 | O | ARG | B | 133 | 10.403 | −9.331 | 44.037 | 1.00 | 67.21 | B |
| ATOM | 4649 | N | PRO | B | 134 | 8.811 | −9.973 | 45.477 | 1.00 | 50.03 | B |
| ATOM | 4650 | CD | PRO | B | 134 | 7.978 | −10.957 | 46.183 | 1.00 | 37.20 | B |
| ATOM | 4651 | CA | PRO | B | 134 | 8.758 | −8.684 | 46.163 | 1.00 | 53.75 | B |
| ATOM | 4652 | CB | PRO | B | 134 | 7.705 | −8.915 | 47.244 | 1.00 | 39.92 | B |
| ATOM | 4653 | CG | PRO | B | 134 | 7.876 | −10.344 | 47.559 | 1.00 | 41.92 | B |
| ATOM | 4654 | C | PRO | B | 134 | 10.092 | −8.257 | 46.739 | 1.00 | 58.93 | B |
| ATOM | 4655 | O | PRO | B | 134 | 10.259 | −7.107 | 47.145 | 1.00 | 61.97 | B |
| ATOM | 4656 | N | ASP | B | 135 | 11.045 | −9.179 | 46.781 | 1.00 | 72.46 | B |
| ATOM | 4657 | CA | ASP | B | 135 | 12.360 | −8.850 | 47.311 | 1.00 | 68.92 | B |
| ATOM | 4658 | CB | ASP | B | 135 | 13.095 | −10.131 | 47.711 | 1.00 | 92.13 | B |
| ATOM | 4659 | CG | ASP | B | 135 | 12.931 | −11.232 | 46.695 | 1.00 | 91.60 | B |
| ATOM | 4660 | OD1 | ASP | B | 135 | 13.576 | −11.154 | 45.633 | 1.00 | 87.54 | B |
| ATOM | 4661 | OD2 | ASP | B | 135 | 12.148 | −12.170 | 46.950 | 1.00 | 93.18 | B |
| ATOM | 4662 | C | ASP | B | 135 | 13.179 | −8.028 | 46.310 | 1.00 | 67.63 | B |
| ATOM | 4663 | O | ASP | B | 135 | 14.198 | −7.443 | 46.670 | 1.00 | 74.32 | B |
| ATOM | 4664 | N | TYR | B | 136 | 12.715 | −7.975 | 45.060 | 1.00 | 59.47 | B |
| ATOM | 4665 | CA | TYR | B | 136 | 13.384 | −7.214 | 44.015 | 1.00 | 60.68 | B |
| ATOM | 4666 | CB | TYR | B | 136 | 13.493 | −8.030 | 42.738 | 1.00 | 80.06 | B |
| ATOM | 4667 | CG | TYR | B | 136 | 14.527 | −9.121 | 42.773 | 1.00 | 79.67 | B |
| ATOM | 4668 | CD1 | TYR | B | 136 | 14.162 | −10.453 | 42.581 | 1.00 | 79.27 | B |
| ATOM | 4669 | CE1 | TYR | B | 136 | 15.114 | −11.470 | 42.547 | 1.00 | 81.82 | B |
| ATOM | 4670 | CD2 | TYR | B | 136 | 15.875 | −8.828 | 42.941 | 1.00 | 81.84 | B |
| ATOM | 4671 | CE2 | TYR | B | 136 | 16.840 | −9.841 | 42.909 | 1.00 | 81.31 | B |
| ATOM | 4672 | CZ | TYR | B | 136 | 16.449 | −11.160 | 42.708 | 1.00 | 82.61 | B |
| ATOM | 4673 | OH | TYR | B | 136 | 17.386 | −12.171 | 42.650 | 1.00 | 86.45 | B |
| ATOM | 4674 | C | TYR | B | 136 | 12.616 | −5.935 | 43.703 | 1.00 | 62.59 | B |
| ATOM | 4675 | O | TYR | B | 136 | 12.956 | −5.204 | 42.778 | 1.00 | 65.47 | B |
| ATOM | 4676 | N | LEU | B | 137 | 11.580 | −5.658 | 44.479 | 1.00 | 72.08 | B |
| ATOM | 4677 | CA | LEU | B | 137 | 10.772 | −4.470 | 44.258 | 1.00 | 68.79 | B |
| ATOM | 4678 | CB | LEU | B | 137 | 9.558 | −4.507 | 45.177 | 1.00 | 68.28 | B |
| ATOM | 4679 | CG | LEU | B | 137 | 8.185 | −4.497 | 44.515 | 1.00 | 66.01 | B |
| ATOM | 4680 | CD1 | LEU | B | 137 | 7.138 | −4.256 | 45.588 | 1.00 | 68.15 | B |
| ATOM | 4681 | CD2 | LEU | B | 137 | 8.107 | −3.412 | 43.462 | 1.00 | 69.77 | B |
| ATOM | 4682 | C | LEU | B | 137 | 11.511 | −3.151 | 44.480 | 1.00 | 68.16 | B |
| ATOM | 4683 | O | LEU | B | 137 | 11.946 | −2.873 | 45.589 | 1.00 | 67.01 | B |
| ATOM | 4684 | N | LEU | B | 138 | 11.648 | −2.334 | 43.439 | 1.00 | 72.27 | B |
| ATOM | 4685 | CA | LEU | B | 138 | 12.298 | −1.034 | 43.603 | 1.00 | 70.07 | B |
| ATOM | 4686 | CB | LEU | B | 138 | 13.247 | −0.696 | 42.442 | 1.00 | 64.75 | B |
| ATOM | 4687 | CG | LEU | B | 138 | 13.695 | 0.784 | 42.460 | 1.00 | 60.87 | B |
| ATOM | 4688 | CD1 | LEU | B | 138 | 14.164 | 1.157 | 43.854 | 1.00 | 62.44 | B |
| ATOM | 4689 | CD2 | LEU | B | 138 | 14.791 | 1.043 | 41.450 | 1.00 | 63.79 | B |
| ATOM | 4690 | C | LEU | B | 138 | 11.262 | 0.069 | 43.699 | 1.00 | 73.42 | B |
| ATOM | 4691 | O | LEU | B | 138 | 10.765 | 0.554 | 42.685 | 1.00 | 72.57 | B |
| ATOM | 4692 | N | PHE | B | 139 | 10.935 | 0.465 | 44.919 | 1.00 | 57.55 | B |
| ATOM | 4693 | CA | PHE | B | 139 | 9.963 | 1.529 | 45.125 | 1.00 | 58.93 | B |
| ATOM | 4694 | CB | PHE | B | 139 | 8.763 | 1.009 | 45.937 | 1.00 | 88.64 | B |
| ATOM | 4695 | CG | PHE | B | 139 | 9.137 | 0.162 | 47.125 | 1.00 | 87.79 | B |
| ATOM | 4696 | CD1 | PHE | B | 139 | 8.269 | −0.822 | 47.581 | 1.00 | 87.32 | B |
| ATOM | 4697 | CD2 | PHE | B | 139 | 10.345 | 0.347 | 47.794 | 1.00 | 90.40 | B |
| ATOM | 4698 | CE1 | PHE | B | 139 | 8.593 | −1.609 | 48.679 | 1.00 | 91.96 | B |
| ATOM | 4699 | CE2 | PHE | B | 139 | 10.679 | −0.434 | 48.894 | 1.00 | 87.94 | B |
| ATOM | 4700 | CZ | PHE | B | 139 | 9.801 | −1.415 | 49.338 | 1.00 | 91.88 | B |
| ATOM | 4701 | C | PHE | B | 139 | 10.657 | 2.682 | 45.834 | 1.00 | 62.96 | B |
| ATOM | 4702 | O | PHE | B | 139 | 10.087 | 3.332 | 46.710 | 1.00 | 63.06 | B |
| ATOM | 4703 | N | ALA | B | 140 | 11.899 | 2.930 | 45.428 | 1.00 | 60.83 | B |
| ATOM | 4704 | CA | ALA | B | 140 | 12.721 | 3.983 | 46.018 | 1.00 | 62.13 | B |
| ATOM | 4705 | CB | ALA | B | 140 | 14.206 | 3.697 | 45.757 | 1.00 | 54.63 | B |
| ATOM | 4706 | C | ALA | B | 140 | 12.376 | 5.368 | 45.512 | 1.00 | 62.52 | B |
| ATOM | 4707 | O | ALA | B | 140 | 12.253 | 6.302 | 46.302 | 1.00 | 64.41 | B |
| ATOM | 4708 | N | GLU | B | 141 | 12.222 | 5.497 | 44.196 | 1.00 | 79.55 | B |
| ATOM | 4709 | CA | GLU | B | 141 | 11.925 | 6.785 | 43.594 | 1.00 | 82.73 | B |
| ATOM | 4710 | CB | GLU | B | 141 | 12.138 | 6.736 | 42.077 | 1.00 | 200.43 | B |
| ATOM | 4711 | CG | GLU | B | 141 | 11.983 | 8.090 | 41.381 | 1.00 | 200.43 | B |
| ATOM | 4712 | CD | GLU | B | 141 | 10.539 | 8.435 | 41.043 | 1.00 | 200.43 | B |
| ATOM | 4713 | OE1 | GLU | B | 141 | 10.221 | 9.638 | 40.942 | 1.00 | 200.43 | B |
| ATOM | 4714 | OE2 | GLU | B | 141 | 9.724 | 7.508 | 40.863 | 1.00 | 200.43 | B |
| ATOM | 4715 | C | GLU | B | 141 | 10.535 | 7.309 | 43.899 | 1.00 | 84.83 | B |
| ATOM | 4716 | O | GLU | B | 141 | 10.379 | 8.132 | 44.794 | 1.00 | 88.51 | B |
| ATOM | 4717 | N | SER | B | 142 | 9.521 | 6.847 | 43.175 | 1.00 | 108.81 | B |
| ATOM | 4718 | CA | SER | B | 142 | 8.182 | 7.365 | 43.416 | 1.00 | 111.80 | B |
| ATOM | 4719 | CB | SER | B | 142 | 8.218 | 8.883 | 43.268 | 1.00 | 47.90 | B |
| ATOM | 4720 | OG | SER | B | 142 | 7.078 | 9.486 | 43.839 | 1.00 | 48.78 | B |
| ATOM | 4721 | C | SER | B | 142 | 7.106 | 6.784 | 42.495 | 1.00 | 113.12 | B |
| ATOM | 4722 | O | SER | B | 142 | 6.333 | 5.905 | 42.895 | 1.00 | 118.21 | B |
| ATOM | 4723 | N | GLY | B | 143 | 7.038 | 7.287 | 41.268 | 1.00 | 54.55 | B |
| ATOM | 4724 | CA | GLY | B | 143 | 6.040 | 6.770 | 40.354 | 1.00 | 52.02 | B |
| ATOM | 4725 | C | GLY | B | 143 | 6.527 | 5.530 | 39.625 | 1.00 | 50.09 | B |
| ATOM | 4726 | O | GLY | B | 143 | 6.232 | 5.353 | 38.447 | 1.00 | 52.08 | B |
| ATOM | 4727 | N | GLN | B | 144 | 7.263 | 4.670 | 40.324 | 1.00 | 50.92 | B |
| ATOM | 4728 | CA | GLN | B | 144 | 7.813 | 3.447 | 39.740 | 1.00 | 45.45 | B |
| ATOM | 4729 | CB | GLN | B | 144 | 9.191 | 3.162 | 40.332 | 1.00 | 75.76 | B |
| ATOM | 4730 | CG | GLN | B | 144 | 10.110 | 4.367 | 40.365 | 1.00 | 75.04 | B |
| ATOM | 4731 | CD | GLN | B | 144 | 11.515 | 4.026 | 40.832 | 1.00 | 75.01 | B |
| ATOM | 4732 | OE1 | GLN | B | 144 | 11.698 | 3.362 | 41.852 | 1.00 | 80.58 | B |
| ATOM | 4733 | NE2 | GLN | B | 144 | 12.517 | 4.495 | 40.093 | 1.00 | 73.55 | B |
| ATOM | 4734 | C | GLN | B | 144 | 6.936 | 2.223 | 39.954 | 1.00 | 47.25 | B |
| ATOM | 4735 | O | GLN | B | 144 | 7.052 | 1.245 | 39.232 | 1.00 | 49.56 | B |
| ATOM | 4736 | N | VAL | B | 145 | 6.071 | 2.268 | 40.960 | 1.00 | 47.92 | B |
| ATOM | 4737 | CA | VAL | B | 145 | 5.189 | 1.150 | 41.255 | 1.00 | 46.17 | B |
| ATOM | 4738 | CB | VAL | B | 145 | 5.719 | 0.352 | 42.462 | 1.00 | 46.27 | B |
| ATOM | 4739 | CG1 | VAL | B | 145 | 4.827 | −0.846 | 42.745 | 1.00 | 44.43 | B |
| ATOM | 4740 | CG2 | VAL | B | 145 | 7.124 | −0.104 | 42.181 | 1.00 | 43.35 | B |
| ATOM | 4741 | C | VAL | B | 145 | 3.789 | 1.669 | 41.556 | 1.00 | 47.28 | B |
| ATOM | 4742 | O | VAL | B | 145 | 3.581 | 2.367 | 42.546 | 1.00 | 49.96 | B |
| ATOM | 4743 | N | TYR | B | 146 | 2.832 | 1.332 | 40.697 | 1.00 | 48.65 | B |
| ATOM | 4744 | CA | TYR | B | 146 | 1.461 | 1.787 | 40.884 | 1.00 | 46.86 | B |
| ATOM | 4745 | CB | TYR | B | 146 | 1.242 | 3.165 | 40.226 | 1.00 | 38.71 | B |
| ATOM | 4746 | CG | TYR | B | 146 | 1.737 | 3.259 | 38.799 | 1.00 | 38.74 | B |
| ATOM | 4747 | CD1 | TYR | B | 146 | 3.067 | 3.564 | 38.522 | 1.00 | 38.13 | B |
| ATOM | 4748 | CE1 | TYR | B | 146 | 3.539 | 3.618 | 37.220 | 1.00 | 36.92 | B |
| ATOM | 4749 | CD2 | TYR | B | 146 | 0.888 | 3.010 | 37.734 | 1.00 | 34.33 | B |
| ATOM | 4750 | CE2 | TYR | B | 146 | 1.346 | 3.059 | 36.420 | 1.00 | 36.38 | B |
| ATOM | 4751 | CZ | TYR | B | 146 | 2.673 | 3.363 | 36.168 | 1.00 | 36.42 | B |
| ATOM | 4752 | OH | TYR | B | 146 | 3.128 | 3.400 | 34.871 | 1.00 | 37.40 | B |
| ATOM | 4753 | C | TYR | B | 146 | 0.440 | 0.793 | 40.354 | 1.00 | 45.17 | B |
| ATOM | 4754 | O | TYR | B | 146 | 0.763 | −0.127 | 39.599 | 1.00 | 43.34 | B |
| ATOM | 4755 | N | PHE | B | 147 | −0.806 | 1.011 | 40.752 | 1.00 | 36.22 | B |
| ATOM | 4756 | CA | PHE | B | 147 | −1.910 | 0.144 | 40.387 | 1.00 | 35.74 | B |
| ATOM | 4757 | CB | PHE | B | 147 | −1.966 | −0.987 | 41.418 | 1.00 | 37.66 | B |
| ATOM | 4758 | CG | PHE | B | 147 | −3.190 | −1.850 | 41.347 | 1.00 | 36.71 | B |
| ATOM | 4759 | CD1 | PHE | B | 147 | −3.770 | −2.182 | 40.131 | 1.00 | 37.20 | B |
| ATOM | 4760 | CD2 | PHE | B | 147 | −3.719 | −2.407 | 42.518 | 1.00 | 39.04 | B |
| ATOM | 4761 | CE1 | PHE | B | 147 | −4.856 | −3.061 | 40.083 | 1.00 | 37.53 | B |
| ATOM | 4762 | CE2 | PHE | B | 147 | −4.801 | −3.285 | 42.479 | 1.00 | 37.61 | B |
| ATOM | 4763 | CZ | PHE | B | 147 | −5.369 | −3.615 | 41.267 | 1.00 | 38.45 | B |
| ATOM | 4764 | C | PHE | B | 147 | −3.150 | 1.028 | 40.432 | 1.00 | 37.28 | B |
| ATOM | 4765 | O | PHE | B | 147 | −3.373 | 1.740 | 41.407 | 1.00 | 34.76 | B |
| ATOM | 4766 | N | GLY | B | 148 | −3.942 | 1.010 | 39.365 | 1.00 | 30.29 | B |
| ATOM | 4767 | CA | GLY | B | 148 | −5.127 | 1.842 | 39.351 | 1.00 | 34.77 | B |
| ATOM | 4768 | C | GLY | B | 148 | −6.219 | 1.364 | 38.423 | 1.00 | 36.99 | B |
| ATOM | 4769 | O | GLY | B | 148 | −6.010 | 0.462 | 37.605 | 1.00 | 39.60 | B |
| ATOM | 4770 | N | ILE | B | 149 | −7.393 | 1.970 | 38.565 | 1.00 | 35.51 | B |
| ATOM | 4771 | CA | ILE | B | 149 | −8.543 | 1.649 | 37.733 | 1.00 | 33.92 | B |
| ATOM | 4772 | CB | ILE | B | 149 | −9.384 | 0.483 | 38.307 | 1.00 | 45.17 | B |
| ATOM | 4773 | CG2 | ILE | B | 149 | −8.500 | −0.693 | 38.651 | 1.00 | 45.72 | B |
| ATOM | 4774 | CG1 | ILE | B | 149 | −10.093 | 0.915 | 39.575 | 1.00 | 41.21 | B |
| ATOM | 4775 | CD1 | ILE | B | 149 | −11.056 | −0.132 | 40.036 | 1.00 | 44.46 | B |
| ATOM | 4776 | C | ILE | B | 149 | −9.429 | 2.874 | 37.638 | 1.00 | 35.96 | B |
| ATOM | 4777 | O | ILE | B | 149 | −9.509 | 3.683 | 38.564 | 1.00 | 41.13 | B |
| ATOM | 4778 | N | ILE | B | 150 | −10.097 | 3.000 | 36.509 | 1.00 | 34.81 | B |
| ATOM | 4779 | CA | ILE | B | 150 | −10.984 | 4.126 | 36.273 | 1.00 | 35.32 | B |
| ATOM | 4780 | CB | ILE | B | 150 | −10.257 | 5.209 | 35.407 | 1.00 | 34.61 | B |
| ATOM | 4781 | CG2 | ILE | B | 150 | −9.717 | 4.594 | 34.139 | 1.00 | 37.91 | B |
| ATOM | 4782 | CG1 | ILE | B | 150 | −11.212 | 6.339 | 35.060 | 1.00 | 32.60 | B |
| ATOM | 4783 | CD1 | ILE | B | 150 | −11.575 | 7.211 | 36.241 | 1.00 | 30.61 | B |
| ATOM | 4784 | C | ILE | B | 150 | −12.213 | 3.567 | 35.546 | 1.00 | 36.66 | B |
| ATOM | 4785 | O | ILE | B | 150 | −12.097 | 2.737 | 34.652 | 1.00 | 35.77 | B |
| ATOM | 4786 | N | ALA | B | 151 | −13.393 | 4.006 | 35.950 | 1.00 | 55.71 | B |
| ATOM | 4787 | CA | ALA | B | 151 | −14.618 | 3.537 | 35.325 | 1.00 | 54.45 | B |
| ATOM | 4788 | CB | ALA | B | 151 | −15.781 | 3.732 | 36.280 | 1.00 | 17.71 | B |
| ATOM | 4789 | C | ALA | B | 151 | −14.868 | 4.303 | 34.030 | 1.00 | 55.41 | B |
| ATOM | 4790 | O | ALA | B | 151 | −14.947 | 5.528 | 34.029 | 1.00 | 57.79 | B |
| ATOM | 4791 | N | LEU | B | 152 | −14.992 | 3.579 | 32.929 | 1.00 | 67.10 | B |
| ATOM | 4792 | CA | LEU | B | 152 | −15.234 | 4.208 | 31.646 | 1.00 | 71.08 | B |
| ATOM | 4793 | CB | LEU | B | 152 | −14.331 | 3.591 | 30.585 | 1.00 | 53.53 | B |
| ATOM | 4794 | CG | LEU | B | 152 | −12.844 | 3.766 | 30.866 | 1.00 | 54.78 | B |
| ATOM | 4795 | CD1 | LEU | B | 152 | −12.027 | 3.083 | 29.794 | 1.00 | 52.47 | B |
| ATOM | 4796 | CD2 | LEU | B | 152 | −12.513 | 5.221 | 30.920 | 1.00 | 53.98 | B |
| ATOM | 4797 | C | LEU | B | 152 | −16.693 | 4.051 | 31.243 | 1.00 | 73.93 | B |
| ATOM | 4798 | O | LEU | B | 152 | −17.448 | 5.025 | 31.233 | 1.00 | 79.79 | B |
| ATOM | 4799 | N | ILE | B | 153 | 2.463 | 9.916 | 61.583 | 1.00 | 39.10 | B |
| ATOM | 4800 | CA | ILE | B | 153 | 3.519 | 10.924 | 61.567 | 1.00 | 39.10 | B |
| ATOM | 4801 | CB | ILE | B | 153 | 4.580 | 10.624 | 60.490 | 1.00 | 27.77 | B |
| ATOM | 4802 | CG2 | ILE | B | 153 | 5.355 | 9.382 | 60.856 | 1.00 | 24.55 | B |
| ATOM | 4803 | CG1 | ILE | B | 153 | 3.907 | 10.457 | 59.131 | 1.00 | 24.55 | B |
| ATOM | 4804 | CD1 | ILE | B | 153 | 4.881 | 10.158 | 58.010 | 1.00 | 24.55 | B |
| ATOM | 4805 | C | ILE | B | 153 | 2.965 | 12.330 | 61.347 | 1.00 | 39.10 | B |
| ATOM | 4806 | O | ILE | B | 153 | 1.879 | 12.521 | 60.804 | 1.00 | 39.10 | B |
| ATOM | 4807 | N | THR | B | 154 | 3.750 | 13.310 | 61.774 | 1.00 | 43.97 | B |
| ATOM | 4808 | CA | THR | B | 154 | 3.364 | 14.705 | 61.713 | 1.00 | 43.97 | B |
| ATOM | 4809 | CB | THR | B | 154 | 2.787 | 15.103 | 63.093 | 1.00 | 43.69 | B |
| ATOM | 4810 | OG1 | THR | B | 154 | 1.360 | 15.135 | 63.011 | 1.00 | 53.97 | B |
| ATOM | 4811 | CG2 | THR | B | 154 | 3.352 | 16.458 | 63.579 | 1.00 | 53.97 | B |
| ATOM | 4812 | C | THR | B | 154 | 4.494 | 15.665 | 61.349 | 1.00 | 43.97 | B |
| ATOM | 4813 | O | THR | B | 154 | 5.649 | 15.458 | 61.729 | 1.00 | 43.97 | B |
| ATOM | 4814 | N | LEU | B | 155 | 4.149 | 16.710 | 60.604 | 1.00 | 40.89 | B |
| ATOM | 4815 | CA | LEU | B | 155 | 5.104 | 17.757 | 60.242 | 1.00 | 40.89 | B |
| ATOM | 4816 | CB | LEU | B | 155 | 5.530 | 17.662 | 58.773 | 1.00 | 41.04 | B |
| ATOM | 4817 | CG | LEU | B | 155 | 6.594 | 18.694 | 58.351 | 1.00 | 37.62 | B |
| ATOM | 4818 | CD1 | LEU | B | 155 | 7.798 | 18.596 | 59.273 | 1.00 | 37.62 | B |
| ATOM | 4819 | CD2 | LEU | B | 155 | 7.014 | 18.457 | 56.910 | 1.00 | 37.62 | B |
| ATOM | 4820 | C | LEU | B | 155 | 4.411 | 19.103 | 60.496 | 1.00 | 40.89 | B |
| ATOM | 4821 | O | LEU | B | 155 | 3.403 | 19.424 | 59.857 | 1.00 | 40.89 | B |
| ATOM | 4822 | N | LYS | B | 156 | 4.939 | 19.882 | 61.437 | 1.00 | 40.91 | B |
| ATOM | 4823 | CA | LYS | B | 156 | 4.329 | 21.157 | 61.773 | 1.00 | 40.91 | B |
| ATOM | 4824 | CB | LYS | B | 156 | 3.725 | 21.084 | 63.176 | 1.00 | 51.07 | B |
| ATOM | 4825 | CG | LYS | B | 156 | 2.951 | 22.338 | 63.563 | 1.00 | 53.26 | B |
| ATOM | 4826 | CD | LYS | B | 156 | 2.344 | 22.267 | 64.966 | 1.00 | 53.26 | B |
| ATOM | 4827 | CE | LYS | B | 156 | 3.395 | 22.303 | 66.081 | 1.00 | 53.26 | B |
| ATOM | 4828 | NZ | LYS | B | 156 | 2.782 | 22.405 | 67.441 | 1.00 | 53.26 | B |
| ATOM | 4829 | C | LYS | B | 156 | 5.291 | 22.338 | 61.706 | 1.00 | 40.91 | B |
| ATOM | 4830 | O | LYS | B | 156 | 6.402 | 22.266 | 62.226 | 1.00 | 40.91 | B |
| ATOM | 4831 | N | TYR | B | 157 | 4.850 | 23.417 | 61.062 | 1.00 | 39.57 | B |
| ATOM | 4832 | CA | TYR | B | 157 | 5.625 | 24.650 | 60.934 | 1.00 | 39.57 | B |
| ATOM | 4833 | CB | TYR | B | 157 | 5.747 | 25.076 | 59.468 | 1.00 | 47.66 | B |
| ATOM | 4834 | CG | TYR | B | 157 | 6.736 | 24.272 | 58.654 | 1.00 | 30.00 | B |
| ATOM | 4835 | CD1 | TYR | B | 157 | 7.370 | 23.158 | 59.188 | 1.00 | 30.00 | B |
| ATOM | 4836 | CE1 | TYR | B | 157 | 8.263 | 22.415 | 58.431 | 1.00 | 30.00 | B |
| ATOM | 4837 | CD2 | TYR | B | 157 | 7.028 | 24.621 | 57.328 | 1.00 | 30.00 | B |
| ATOM | 4838 | CE2 | TYR | B | 157 | 7.927 | 23.878 | 56.562 | 1.00 | 30.00 | B |
| ATOM | 4839 | CZ | TYR | B | 157 | 8.537 | 22.782 | 57.124 | 1.00 | 30.00 | B |
| ATOM | 4840 | OH | TYR | B | 157 | 9.433 | 22.051 | 56.376 | 1.00 | 30.00 | B |
| ATOM | 4841 | C | TYR | B | 157 | 4.880 | 25.733 | 61.719 | 1.00 | 39.57 | B |
| ATOM | 4842 | O | TYR | B | 157 | 3.677 | 25.930 | 61.531 | 1.00 | 39.57 | B |
| ATOM | 4843 | N | ASN | B | 158 | 5.594 | 26.433 | 62.594 | 1.00 | 40.67 | B |
| ATOM | 4844 | CA | ASN | B | 158 | 4.993 | 27.480 | 63.417 | 1.00 | 40.67 | B |
| ATOM | 4845 | CB | ASN | B | 158 | 4.948 | 27.002 | 64.880 | 1.00 | 26.81 | B |
| ATOM | 4846 | CG | ASN | B | 158 | 4.308 | 28.004 | 65.832 | 1.00 | 37.51 | B |
| ATOM | 4847 | OD1 | ASN | B | 158 | 3.932 | 29.114 | 65.449 | 1.00 | 37.51 | B |
| ATOM | 4848 | ND2 | ASN | B | 158 | 4.192 | 27.594 | 67.092 | 1.00 | 37.51 | B |
| ATOM | 4849 | C | ASN | B | 158 | 5.796 | 28.774 | 63.315 | 1.00 | 40.67 | B |
| ATOM | 4850 | O | ASN | B | 158 | 6.955 | 28.837 | 63.749 | 1.00 | 40.67 | B |
| ATOM | 4851 | N | TYR | B | 159 | 5.192 | 29.797 | 62.717 | 1.00 | 31.83 | B |
| ATOM | 4852 | CA | TYR | B | 159 | 5.846 | 31.100 | 62.616 | 1.00 | 31.83 | B |
| ATOM | 4853 | CB | TYR | B | 159 | 5.708 | 31.700 | 61.220 | 1.00 | 31.53 | B |
| ATOM | 4854 | CG | TYR | B | 159 | 6.373 | 30.929 | 60.115 | 1.00 | 35.09 | B |
| ATOM | 4855 | CD1 | TYR | B | 159 | 5.741 | 29.841 | 59.526 | 1.00 | 35.09 | B |
| ATOM | 4856 | CE1 | TYR | B | 159 | 6.301 | 29.187 | 58.444 | 1.00 | 35.09 | B |
| ATOM | 4857 | CD2 | TYR | B | 159 | 7.600 | 31.337 | 59.597 | 1.00 | 35.09 | B |
| ATOM | 4858 | CE2 | TYR | B | 159 | 8.171 | 30.687 | 58.515 | 1.00 | 35.09 | B |
| ATOM | 4859 | CZ | TYR | B | 159 | 7.510 | 29.614 | 57.941 | 1.00 | 35.09 | B |
| ATOM | 4860 | OH | TYR | B | 159 | 8.048 | 28.987 | 56.837 | 1.00 | 35.09 | B |
| ATOM | 4861 | C | TYR | B | 159 | 5.144 | 32.030 | 63.586 | 1.00 | 31.83 | B |
| ATOM | 4862 | O | TYR | B | 159 | 3.914 | 32.146 | 63.538 | 1.00 | 31.83 | B |
| ATOM | 4863 | N | THR | B | 160 | 5.901 | 32.706 | 64.448 | 1.00 | 43.56 | B |
| ATOM | 4864 | CA | THR | B | 160 | 5.267 | 33.609 | 65.403 | 1.00 | 43.56 | B |
| ATOM | 4865 | CB | THR | B | 160 | 5.096 | 32.916 | 66.771 | 1.00 | 52.25 | B |
| ATOM | 4866 | OG1 | THR | B | 160 | 4.406 | 31.668 | 66.605 | 1.00 | 39.59 | B |
| ATOM | 4867 | CG2 | THR | B | 160 | 4.309 | 33.793 | 67.701 | 1.00 | 39.59 | B |
| ATOM | 4868 | C | THR | B | 160 | 5.971 | 34.947 | 65.645 | 1.00 | 43.56 | B |
| ATOM | 4869 | O | THR | B | 160 | 7.176 | 34.976 | 65.947 | 1.00 | 43.56 | B |
| ATOM | 4870 | N | VAL | B | 161 | 5.239 | 36.055 | 65.484 | 1.00 | 42.79 | B |
| ATOM | 4871 | CA | VAL | B | 161 | 5.812 | 37.372 | 65.798 | 1.00 | 42.79 | B |
| ATOM | 4872 | CB | VAL | B | 161 | 5.712 | 38.382 | 64.646 | 1.00 | 56.67 | B |
| ATOM | 4873 | CG1 | VAL | B | 161 | 6.238 | 37.765 | 63.378 | 1.00 | 41.18 | B |
| ATOM | 4874 | CG2 | VAL | B | 161 | 4.309 | 38.841 | 64.481 | 1.00 | 41.18 | B |
| ATOM | 4875 | C | VAL | B | 161 | 5.031 | 37.892 | 67.009 | 1.00 | 42.79 | B |
| ATOM | 4876 | O | VAL | B | 161 | 3.805 | 37.768 | 67.072 | 1.00 | 42.79 | B |
| ATOM | 4877 | N | THR | B | 162 | 5.747 | 38.447 | 67.979 | 1.00 | 49.90 | B |
| ATOM | 4878 | CA | THR | B | 162 | 5.121 | 38.941 | 69.199 | 1.00 | 49.90 | B |
| ATOM | 4879 | CB | THR | B | 162 | 5.582 | 38.112 | 70.416 | 1.00 | 83.68 | B |
| ATOM | 4880 | OG1 | THR | B | 162 | 5.743 | 36.748 | 70.026 | 1.00 | 55.01 | B |
| ATOM | 4881 | CG2 | THR | B | 162 | 4.559 | 38.167 | 71.522 | 1.00 | 55.01 | B |
| ATOM | 4882 | C | THR | B | 162 | 5.495 | 40.389 | 69.453 | 1.00 | 49.90 | B |
| ATOM | 4883 | O | THR | B | 162 | 6.580 | 40.829 | 69.061 | 1.00 | 49.90 | B |
| ATOM | 4884 | N | LEU | B | 163 | 4.606 | 41.133 | 70.110 | 1.00 | 71.09 | B |
| ATOM | 4885 | CA | LEU | B | 163 | 4.894 | 42.524 | 70.423 | 1.00 | 71.09 | B |
| ATOM | 4886 | CB | LEU | B | 163 | 3.614 | 43.301 | 70.681 | 1.00 | 109.26 | B |
| ATOM | 4887 | CG | LEU | B | 163 | 3.482 | 44.542 | 69.802 | 1.00 | 57.35 | B |
| ATOM | 4888 | CD1 | LEU | B | 163 | 2.302 | 45.352 | 70.287 | 1.00 | 57.35 | B |
| ATOM | 4889 | CD2 | LEU | B | 163 | 4.741 | 45.389 | 69.842 | 1.00 | 57.35 | B |
| ATOM | 4890 | C | LEU | B | 163 | 5.787 | 42.587 | 71.656 | 1.00 | 71.09 | B |
| ATOM | 4891 | O | LEU | B | 163 | 6.298 | 43.648 | 72.029 | 1.00 | 71.09 | B |
| ATOM | 4892 | N | LYS | B | 164 | 5.994 | 41.436 | 72.281 | 1.00 | 178.73 | B |
| ATOM | 4893 | CA | LYS | B | 164 | 6.829 | 41.384 | 73.467 | 1.00 | 178.73 | B |
| ATOM | 4894 | CB | LYS | B | 164 | 8.303 | 41.538 | 73.079 | 1.00 | 52.39 | B |
| ATOM | 4895 | CG | LYS | B | 164 | 9.063 | 40.225 | 73.097 | 1.00 | 52.14 | B |
| ATOM | 4896 | CD | LYS | B | 164 | 9.038 | 39.593 | 74.485 | 1.00 | 52.14 | B |
| ATOM | 4897 | CE | LYS | B | 164 | 9.827 | 38.285 | 74.552 | 1.00 | 52.14 | B |
| ATOM | 4898 | NZ | LYS | B | 164 | 9.961 | 37.758 | 75.971 | 1.00 | 52.14 | B |
| ATOM | 4899 | C | LYS | B | 164 | 6.411 | 42.510 | 74.396 | 1.00 | 178.73 | B |
| ATOM | 4900 | O | LYS | B | 164 | 5.313 | 43.050 | 74.257 | 1.00 | 178.73 | B |
| ATOM | 4901 | N | ASP | B | 165 | 7.271 | 42.861 | 75.348 | 1.00 | 149.49 | B |
| ATOM | 4902 | CA | ASP | B | 165 | 6.954 | 43.945 | 76.263 | 1.00 | 149.49 | B |
| ATOM | 4903 | CB | ASP | B | 165 | 8.053 | 44.100 | 77.322 | 1.00 | 163.18 | B |
| ATOM | 4904 | CG | ASP | B | 165 | 9.447 | 44.130 | 76.721 | 1.00 | 163.18 | B |
| ATOM | 4905 | OD1 | ASP | B | 165 | 9.754 | 45.082 | 75.966 | 1.00 | 163.18 | B |
| ATOM | 4906 | OD2 | ASP | B | 165 | 10.232 | 43.195 | 77.007 | 1.00 | 163.18 | B |
| ATOM | 4907 | C | ASP | B | 165 | 6.831 | 45.204 | 75.417 | 1.00 | 149.49 | B |
| ATOM | 4908 | O | ASP | B | 165 | 6.187 | 45.192 | 74.363 | 1.00 | 149.49 | B |
| ATOM | 4909 | N | ASP | B | 166 | 7.453 | 46.291 | 75.851 | 1.00 | 65.56 | B |
| ATOM | 4910 | CA | ASP | B | 166 | 7.352 | 47.509 | 75.067 | 1.00 | 65.56 | B |
| ATOM | 4911 | CB | ASP | B | 166 | 7.243 | 48.725 | 75.993 | 1.00 | 200.84 | B |
| ATOM | 4912 | CG | ASP | B | 166 | 5.959 | 48.719 | 76.808 | 1.00 | 200.84 | B |
| ATOM | 4913 | OD1 | ASP | B | 166 | 5.754 | 47.766 | 77.590 | 1.00 | 200.84 | B |
| ATOM | 4914 | OD2 | ASP | B | 166 | 5.155 | 49.665 | 76.664 | 1.00 | 200.84 | B |
| ATOM | 4915 | C | ASP | B | 166 | 8.484 | 47.693 | 74.052 | 1.00 | 65.56 | B |
| ATOM | 4916 | O | ASP | B | 166 | 9.664 | 47.727 | 74.407 | 1.00 | 65.56 | B |
| ATOM | 4917 | N | GLY | B | 167 | 8.104 | 47.770 | 72.777 | 1.00 | 60.37 | B |
| ATOM | 4918 | CA | GLY | B | 167 | 9.086 | 47.979 | 71.744 | 1.00 | 60.37 | B |
| ATOM | 4919 | C | GLY | B | 167 | 8.958 | 47.240 | 70.424 | 1.00 | 60.37 | B |
| ATOM | 4920 | O | GLY | B | 167 | 7.989 | 47.380 | 69.660 | 1.00 | 60.37 | B |
| ATOM | 4921 | N | LEU | B | 168 | 9.996 | 46.455 | 70.167 | 1.00 | 49.81 | B |
| ATOM | 4922 | CA | LEU | B | 168 | 10.144 | 45.680 | 68.955 | 1.00 | 49.81 | B |
| ATOM | 4923 | CB | LEU | B | 168 | 11.603 | 45.223 | 68.873 | 1.00 | 51.56 | B |
| ATOM | 4924 | CG | LEU | B | 168 | 12.576 | 46.206 | 69.551 | 1.00 | 51.56 | B |
| ATOM | 4925 | CD1 | LEU | B | 168 | 13.979 | 45.635 | 69.559 | 1.00 | 51.56 | B |
| ATOM | 4926 | CD2 | LEU | B | 168 | 12.547 | 47.541 | 68.839 | 1.00 | 51.56 | B |
| ATOM | 4927 | C | LEU | B | 168 | 9.207 | 44.478 | 68.856 | 1.00 | 49.81 | B |
| ATOM | 4928 | O | LEU | B | 168 | 8.479 | 44.137 | 69.792 | 1.00 | 49.81 | B |
| ATOM | 4929 | N | TYR | B | 169 | 9.222 | 43.851 | 67.688 | 1.00 | 32.54 | B |
| ATOM | 4930 | CA | TYR | B | 169 | 8.430 | 42.657 | 67.442 | 1.00 | 32.54 | B |
| ATOM | 4931 | CB | TYR | B | 169 | 7.649 | 42.775 | 66.138 | 1.00 | 57.17 | B |
| ATOM | 4932 | CG | TYR | B | 169 | 6.543 | 43.799 | 66.160 | 1.00 | 57.17 | B |
| ATOM | 4933 | CD1 | TYR | B | 169 | 6.820 | 45.167 | 66.052 | 1.00 | 57.17 | B |
| ATOM | 4934 | CE1 | TYR | B | 169 | 5.798 | 46.108 | 66.072 | 1.00 | 57.17 | B |
| ATOM | 4935 | CD2 | TYR | B | 169 | 5.214 | 43.403 | 66.289 | 1.00 | 57.17 | B |
| ATOM | 4936 | CE2 | TYR | B | 169 | 4.188 | 44.333 | 66.310 | 1.00 | 57.17 | B |
| ATOM | 4937 | CZ | TYR | B | 169 | 4.487 | 45.681 | 66.201 | 1.00 | 57.17 | B |
| ATOM | 4938 | OH | TYR | B | 169 | 3.468 | 46.600 | 66.224 | 1.00 | 57.17 | B |
| ATOM | 4939 | C | TYR | B | 169 | 9.400 | 41.484 | 67.321 | 1.00 | 32.54 | B |
| ATOM | 4940 | O | TYR | B | 169 | 10.307 | 41.503 | 66.484 | 1.00 | 32.54 | B |
| ATOM | 4941 | N | ASP | B | 170 | 9.224 | 40.476 | 68.168 | 1.00 | 46.78 | B |
| ATOM | 4942 | CA | ASP | B | 170 | 10.082 | 39.314 | 68.105 | 1.00 | 46.78 | B |
| ATOM | 4943 | CB | ASP | B | 170 | 10.209 | 38.651 | 69.480 | 1.00 | 72.79 | B |
| ATOM | 4944 | CG | ASP | B | 170 | 11.411 | 39.155 | 70.249 | 1.00 | 72.79 | B |
| ATOM | 4945 | OD1 | ASP | B | 170 | 12.442 | 39.415 | 69.597 | 1.00 | 72.79 | B |
| ATOM | 4946 | OD2 | ASP | B | 170 | 11.340 | 39.277 | 71.489 | 1.00 | 72.79 | B |
| ATOM | 4947 | C | ASP | B | 170 | 9.523 | 38.330 | 67.100 | 1.00 | 46.78 | B |
| ATOM | 4948 | O | ASP | B | 170 | 8.331 | 38.325 | 66.820 | 1.00 | 46.78 | B |
| ATOM | 4949 | N | GLY | B | 171 | 10.393 | 37.509 | 66.535 | 1.00 | 36.17 | B |
| ATOM | 4950 | CA | GLY | B | 171 | 9.939 | 36.510 | 65.589 | 1.00 | 36.17 | B |
| ATOM | 4951 | C | GLY | B | 171 | 10.642 | 35.182 | 65.813 | 1.00 | 36.17 | B |
| ATOM | 4952 | O | GLY | B | 171 | 11.855 | 35.147 | 66.022 | 1.00 | 36.17 | B |
| ATOM | 4953 | N | VAL | B | 172 | 9.869 | 34.099 | 65.807 | 1.00 | 39.26 | B |
| ATOM | 4954 | CA | VAL | B | 172 | 10.416 | 32.765 | 65.950 | 1.00 | 39.26 | B |
| ATOM | 4955 | CB | VAL | B | 172 | 10.215 | 32.151 | 67.355 | 1.00 | 32.02 | B |
| ATOM | 4956 | CG1 | VAL | B | 172 | 11.316 | 32.557 | 68.253 | 1.00 | 24.03 | B |
| ATOM | 4957 | CG2 | VAL | B | 172 | 8.854 | 32.531 | 67.909 | 1.00 | 24.03 | B |
| ATOM | 4958 | C | VAL | B | 172 | 9.730 | 31.828 | 64.984 | 1.00 | 39.26 | B |
| ATOM | 4959 | O | VAL | B | 172 | 8.595 | 32.060 | 64.553 | 1.00 | 39.26 | B |
| ATOM | 4960 | N | PHE | B | 173 | 10.427 | 30.742 | 64.677 | 1.00 | 37.69 | B |
| ATOM | 4961 | CA | PHE | B | 173 | 9.923 | 29.715 | 63.798 | 1.00 | 37.69 | B |
| ATOM | 4962 | CB | PHE | B | 173 | 10.499 | 29.883 | 62.407 | 1.00 | 53.31 | B |
| ATOM | 4963 | CG | PHE | B | 173 | 10.262 | 28.703 | 61.538 | 1.00 | 35.13 | B |
| ATOM | 4964 | CD1 | PHE | B | 173 | 9.015 | 28.498 | 60.965 | 1.00 | 35.13 | B |
| ATOM | 4965 | CD2 | PHE | B | 173 | 11.260 | 27.747 | 61.364 | 1.00 | 35.13 | B |
| ATOM | 4966 | CE1 | PHE | B | 173 | 8.757 | 27.359 | 60.237 | 1.00 | 35.13 | B |
| ATOM | 4967 | CE2 | PHE | B | 173 | 11.017 | 26.606 | 60.641 | 1.00 | 35.13 | B |
| ATOM | 4968 | CZ | PHE | B | 173 | 9.755 | 26.404 | 60.072 | 1.00 | 35.13 | B |
| ATOM | 4969 | C | PHE | B | 173 | 10.345 | 28.353 | 64.345 | 1.00 | 37.69 | B |
| ATOM | 4970 | O | PHE | B | 173 | 11.486 | 28.169 | 64.762 | 1.00 | 37.69 | B |
| ATOM | 4971 | N | TYR | B | 174 | 9.418 | 27.403 | 64.347 | 1.00 | 37.35 | B |
| ATOM | 4972 | CA | TYR | B | 174 | 9.721 | 26.066 | 64.812 | 1.00 | 37.35 | B |
| ATOM | 4973 | CB | TYR | B | 174 | 8.997 | 25.751 | 66.110 | 1.00 | 57.09 | B |
| ATOM | 4974 | CG | TYR | B | 174 | 9.461 | 26.552 | 67.281 | 1.00 | 43.13 | B |
| ATOM | 4975 | CD1 | TYR | B | 174 | 8.679 | 27.592 | 67.787 | 1.00 | 43.13 | B |
| ATOM | 4976 | CE1 | TYR | B | 174 | 9.096 | 28.335 | 68.898 | 1.00 | 43.13 | B |
| ATOM | 4977 | CD2 | TYR | B | 174 | 10.676 | 26.271 | 67.906 | 1.00 | 43.13 | B |
| ATOM | 4978 | CE2 | TYR | B | 174 | 11.102 | 27.006 | 69.014 | 1.00 | 43.13 | B |
| ATOM | 4979 | CZ | TYR | B | 174 | 10.307 | 28.034 | 69.504 | 1.00 | 43.13 | B |
| ATOM | 4980 | OH | TYR | B | 174 | 10.709 | 28.763 | 70.599 | 1.00 | 43.13 | B |
| ATOM | 4981 | C | TYR | B | 174 | 9.281 | 25.062 | 63.774 | 1.00 | 37.35 | B |
| ATOM | 4982 | O | TYR | B | 174 | 8.194 | 25.191 | 63.213 | 1.00 | 37.35 | B |
| ATOM | 4983 | N | ASP | B | 175 | 10.134 | 24.059 | 63.542 | 1.00 | 41.75 | B |
| ATOM | 4984 | CA | ASP | B | 175 | 9.889 | 22.965 | 62.600 | 1.00 | 41.75 | B |
| ATOM | 4985 | CB | ASP | B | 175 | 11.046 | 22.863 | 61.604 | 1.00 | 56.86 | B |
| ATOM | 4986 | CG | ASP | B | 175 | 10.810 | 21.823 | 60.533 | 1.00 | 45.07 | B |
| ATOM | 4987 | OD1 | ASP | B | 175 | 10.140 | 20.809 | 60.825 | 1.00 | 45.07 | B |
| ATOM | 4988 | OD2 | ASP | B | 175 | 11.309 | 22.008 | 59.403 | 1.00 | 45.07 | B |
| ATOM | 4989 | C | ASP | B | 175 | 9.843 | 21.711 | 63.468 | 1.00 | 41.75 | B |
| ATOM | 4990 | O | ASP | B | 175 | 10.846 | 21.324 | 64.070 | 1.00 | 41.75 | B |
| ATOM | 4991 | N | HIS | B | 176 | 8.681 | 21.082 | 63.551 | 1.00 | 42.80 | B |
| ATOM | 4992 | CA | HIS | B | 176 | 8.538 | 19.879 | 64.368 | 1.00 | 42.80 | B |
| ATOM | 4993 | CB | HIS | B | 176 | 7.508 | 20.105 | 65.471 | 1.00 | 84.22 | B |
| ATOM | 4994 | CG | HIS | B | 176 | 7.997 | 20.939 | 66.611 | 1.00 | 53.35 | B |
| ATOM | 4995 | CD2 | HIS | B | 176 | 7.667 | 22.193 | 67.003 | 1.00 | 53.35 | B |
| ATOM | 4996 | ND1 | HIS | B | 176 | 8.902 | 20.475 | 67.539 | 1.00 | 53.35 | B |
| ATOM | 4997 | CE1 | HIS | B | 176 | 9.105 | 21.404 | 68.457 | 1.00 | 53.35 | B |
| ATOM | 4998 | NE2 | HIS | B | 176 | 8.366 | 22.455 | 68.155 | 1.00 | 53.35 | B |
| ATOM | 4999 | C | HIS | B | 176 | 8.094 | 18.664 | 63.562 | 1.00 | 42.80 | B |
| ATOM | 5000 | O | HIS | B | 176 | 7.097 | 18.715 | 62.838 | 1.00 | 42.80 | B |
| ATOM | 5001 | N | TYR | B | 177 | 8.849 | 17.577 | 63.679 | 1.00 | 42.41 | B |
| ATOM | 5002 | CA | TYR | B | 177 | 8.494 | 16.322 | 63.024 | 1.00 | 42.41 | B |
| ATOM | 5003 | CB | TYR | B | 177 | 9.713 | 15.650 | 62.410 | 1.00 | 54.84 | B |
| ATOM | 5004 | CG | TYR | B | 177 | 9.357 | 14.311 | 61.841 | 1.00 | 47.85 | B |
| ATOM | 5005 | CD1 | TYR | B | 177 | 8.525 | 14.213 | 60.724 | 1.00 | 47.85 | B |
| ATOM | 5006 | CE1 | TYR | B | 177 | 8.143 | 12.978 | 60.211 | 1.00 | 47.85 | B |
| ATOM | 5007 | CD2 | TYR | B | 177 | 9.804 | 13.135 | 62.437 | 1.00 | 47.85 | B |
| ATOM | 5008 | CE2 | TYR | B | 177 | 9.430 | 11.897 | 61.936 | 1.00 | 47.85 | B |
| ATOM | 5009 | CZ | TYR | B | 177 | 8.597 | 11.824 | 60.821 | 1.00 | 47.85 | B |
| ATOM | 5010 | OH | TYR | B | 177 | 8.207 | 10.604 | 60.309 | 1.00 | 47.85 | B |
| ATOM | 5011 | C | TYR | B | 177 | 8.003 | 15.501 | 64.206 | 1.00 | 42.41 | B |
| ATOM | 5012 | O | TYR | B | 177 | 8.782 | 15.191 | 65.090 | 1.00 | 42.41 | B |
| ATOM | 5013 | N | ASN | B | 178 | 6.725 | 15.146 | 64.236 | 1.00 | 40.24 | B |
| ATOM | 5014 | CA | ASN | B | 178 | 6.191 | 14.425 | 65.394 | 1.00 | 40.24 | B |
| ATOM | 5015 | CB | ASN | B | 178 | 6.925 | 13.102 | 65.658 | 1.00 | 55.50 | B |
| ATOM | 5016 | CG | ASN | B | 178 | 6.533 | 12.001 | 64.679 | 1.00 | 47.97 | B |
| ATOM | 5017 | OD1 | ASN | B | 178 | 5.572 | 12.137 | 63.920 | 1.00 | 47.97 | B |
| ATOM | 5018 | ND2 | ASN | B | 178 | 7.271 | 10.897 | 64.705 | 1.00 | 47.97 | B |
| ATOM | 5019 | C | ASN | B | 178 | 6.464 | 15.387 | 66.540 | 1.00 | 40.24 | B |
| ATOM | 5020 | O | ASN | B | 178 | 6.244 | 16.583 | 66.395 | 1.00 | 40.24 | B |
| ATOM | 5021 | N | ASP | B | 179 | 6.952 | 14.889 | 67.667 | 1.00 | 41.89 | B |
| ATOM | 5022 | CA | ASP | B | 179 | 7.245 | 15.785 | 68.780 | 1.00 | 41.89 | B |
| ATOM | 5023 | CB | ASP | B | 179 | 6.839 | 15.146 | 70.125 | 1.00 | 42.10 | B |
| ATOM | 5024 | CG | ASP | B | 179 | 7.472 | 13.782 | 70.356 | 1.00 | 46.05 | B |
| ATOM | 5025 | OD1 | ASP | B | 179 | 8.253 | 13.323 | 69.493 | 1.00 | 46.05 | B |
| ATOM | 5026 | OD2 | ASP | B | 179 | 7.182 | 13.165 | 71.410 | 1.00 | 46.05 | B |
| ATOM | 5027 | C | ASP | B | 179 | 8.720 | 16.183 | 68.804 | 1.00 | 41.89 | B |
| ATOM | 5028 | O | ASP | B | 179 | 9.195 | 16.733 | 69.787 | 1.00 | 41.89 | B |
| ATOM | 5029 | N | GLN | B | 180 | 9.430 | 15.921 | 67.710 | 1.00 | 38.51 | B |
| ATOM | 5030 | CA | GLN | B | 180 | 10.855 | 16.231 | 67.603 | 1.00 | 38.51 | B |
| ATOM | 5031 | CB | GLN | B | 180 | 11.562 | 15.167 | 66.768 | 1.00 | 58.73 | B |
| ATOM | 5032 | CG | GLN | B | 180 | 11.363 | 13.762 | 67.242 | 1.00 | 50.36 | B |
| ATOM | 5033 | CD | GLN | B | 180 | 11.835 | 13.575 | 68.656 | 1.00 | 50.36 | B |
| ATOM | 5034 | OE1 | GLN | B | 180 | 11.035 | 13.555 | 69.592 | 1.00 | 50.36 | B |
| ATOM | 5035 | NE2 | GLN | B | 180 | 13.150 | 13.452 | 68.827 | 1.00 | 50.36 | B |
| ATOM | 5036 | C | GLN | B | 180 | 11.169 | 17.588 | 66.973 | 1.00 | 38.51 | B |
| ATOM | 5037 | O | GLN | B | 180 | 10.811 | 17.838 | 65.822 | 1.00 | 38.51 | B |
| ATOM | 5038 | N | LEU | B | 181 | 11.852 | 18.457 | 67.714 | 1.00 | 37.88 | B |
| ATOM | 5039 | CA | LEU | B | 181 | 12.234 | 19.755 | 67.181 | 1.00 | 37.88 | B |
| ATOM | 5040 | CB | LEU | B | 181 | 12.824 | 20.632 | 68.278 | 1.00 | 45.98 | B |
| ATOM | 5041 | CG | LEU | B | 181 | 13.364 | 21.962 | 67.755 | 1.00 | 33.41 | B |
| ATOM | 5042 | CD1 | LEU | B | 181 | 12.236 | 22.771 | 67.113 | 1.00 | 33.41 | B |
| ATOM | 5043 | CD2 | LEU | B | 181 | 14.003 | 22.732 | 68.889 | 1.00 | 33.41 | B |
| ATOM | 5044 | C | LEU | B | 181 | 13.277 | 19.516 | 66.087 | 1.00 | 37.88 | B |
| ATOM | 5045 | O | LEU | B | 181 | 14.245 | 18.788 | 66.285 | 1.00 | 37.88 | B |
| ATOM | 5046 | N | VAL | B | 182 | 13.073 | 20.118 | 64.924 | 1.00 | 40.21 | B |
| ATOM | 5047 | CA | VAL | B | 182 | 13.999 | 19.945 | 63.823 | 1.00 | 40.21 | B |
| ATOM | 5048 | CB | VAL | B | 182 | 13.253 | 19.562 | 62.543 | 1.00 | 46.95 | B |
| ATOM | 5049 | CG1 | VAL | B | 182 | 14.234 | 19.445 | 61.389 | 1.00 | 25.43 | B |
| ATOM | 5050 | CG2 | VAL | B | 182 | 12.503 | 18.269 | 62.752 | 1.00 | 25.43 | B |
| ATOM | 5051 | C | VAL | B | 182 | 14.771 | 21.227 | 63.560 | 1.00 | 40.21 | B |
| ATOM | 5052 | O | VAL | B | 182 | 15.975 | 21.199 | 63.283 | 1.00 | 40.21 | B |
| ATOM | 5053 | N | THR | B | 183 | 14.071 | 22.353 | 63.644 | 1.00 | 40.06 | B |
| ATOM | 5054 | CA | THR | B | 183 | 14.687 | 23.643 | 63.389 | 1.00 | 40.06 | B |
| ATOM | 5055 | CB | THR | B | 183 | 14.549 | 24.042 | 61.903 | 1.00 | 56.68 | B |
| ATOM | 5056 | OG1 | THR | B | 183 | 15.291 | 23.132 | 61.086 | 1.00 | 43.27 | B |
| ATOM | 5057 | CG2 | THR | B | 183 | 15.050 | 25.460 | 61.681 | 1.00 | 43.27 | B |
| ATOM | 5058 | C | THR | B | 183 | 14.065 | 24.763 | 64.194 | 1.00 | 40.06 | B |
| ATOM | 5059 | O | THR | B | 183 | 12.849 | 24.795 | 64.376 | 1.00 | 40.06 | B |
| ATOM | 5060 | N | LYS | B | 184 | 14.906 | 25.667 | 64.690 | 1.00 | 38.10 | B |
| ATOM | 5061 | CA | LYS | B | 184 | 14.423 | 26.834 | 65.402 | 1.00 | 38.10 | B |
| ATOM | 5062 | CB | LYS | B | 184 | 14.765 | 26.810 | 66.874 | 1.00 | 42.37 | B |
| ATOM | 5063 | CG | LYS | B | 184 | 14.306 | 28.088 | 67.553 | 1.00 | 34.21 | B |
| ATOM | 5064 | CD | LYS | B | 184 | 14.651 | 28.133 | 69.028 | 1.00 | 34.21 | B |
| ATOM | 5065 | CE | LYS | B | 184 | 14.194 | 29.447 | 69.654 | 1.00 | 34.21 | B |
| ATOM | 5066 | NZ | LYS | B | 184 | 14.659 | 29.592 | 71.064 | 1.00 | 34.21 | B |
| ATOM | 5067 | C | LYS | B | 184 | 15.113 | 28.027 | 64.772 | 1.00 | 38.10 | B |
| ATOM | 5068 | O | LYS | B | 184 | 16.331 | 28.007 | 64.526 | 1.00 | 38.10 | B |
| ATOM | 5069 | N | ILE | B | 185 | 14.327 | 29.057 | 64.479 | 1.00 | 42.04 | B |
| ATOM | 5070 | CA | ILE | B | 185 | 14.854 | 30.268 | 63.878 | 1.00 | 42.04 | B |
| ATOM | 5071 | CB | ILE | B | 185 | 14.487 | 30.353 | 62.383 | 1.00 | 56.32 | B |
| ATOM | 5072 | CG2 | ILE | B | 185 | 14.808 | 31.710 | 61.840 | 1.00 | 25.24 | B |
| ATOM | 5073 | CG1 | ILE | B | 185 | 15.294 | 29.333 | 61.595 | 1.00 | 25.24 | B |
| ATOM | 5074 | CD1 | ILE | B | 185 | 14.871 | 29.222 | 60.142 | 1.00 | 25.24 | B |
| ATOM | 5075 | C | ILE | B | 185 | 14.267 | 31.452 | 64.614 | 1.00 | 42.04 | B |
| ATOM | 5076 | O | ILE | B | 185 | 13.098 | 31.441 | 64.990 | 1.00 | 42.04 | B |
| ATOM | 5077 | N | SER | B | 186 | 15.087 | 32.466 | 64.859 | 1.00 | 45.52 | B |
| ATOM | 5078 | CA | SER | B | 186 | 14.597 | 33.660 | 65.531 | 1.00 | 45.52 | B |
| ATOM | 5079 | CB | SER | B | 186 | 14.940 | 33.637 | 67.017 | 1.00 | 72.85 | B |
| ATOM | 5080 | OG | SER | B | 186 | 16.272 | 33.250 | 67.218 | 1.00 | 61.96 | B |
| ATOM | 5081 | C | SER | B | 186 | 15.141 | 34.910 | 64.865 | 1.00 | 45.52 | B |
| ATOM | 5082 | O | SER | B | 186 | 16.218 | 34.885 | 64.254 | 1.00 | 45.52 | B |
| ATOM | 5083 | N | TYR | B | 187 | 14.367 | 35.991 | 64.959 | 1.00 | 36.43 | B |
| ATOM | 5084 | CA | TYR | B | 187 | 14.733 | 37.248 | 64.345 | 1.00 | 36.43 | B |
| ATOM | 5085 | CB | TYR | B | 187 | 13.509 | 38.136 | 64.210 | 1.00 | 77.63 | B |
| ATOM | 5086 | CG | TYR | B | 187 | 13.789 | 39.401 | 63.447 | 1.00 | 31.84 | B |
| ATOM | 5087 | CD1 | TYR | B | 187 | 14.011 | 39.374 | 62.073 | 1.00 | 31.84 | B |
| ATOM | 5088 | CE1 | TYR | B | 187 | 14.269 | 40.537 | 61.364 | 1.00 | 31.84 | B |
| ATOM | 5089 | CD2 | TYR | B | 187 | 13.841 | 40.630 | 64.096 | 1.00 | 31.84 | B |
| ATOM | 5090 | CE2 | TYR | B | 187 | 14.106 | 41.806 | 63.400 | 1.00 | 31.84 | B |
| ATOM | 5091 | CZ | TYR | B | 187 | 14.319 | 41.754 | 62.038 | 1.00 | 31.84 | B |
| ATOM | 5092 | OH | TYR | B | 187 | 14.613 | 42.908 | 61.354 | 1.00 | 31.84 | B |
| ATOM | 5093 | C | TYR | B | 187 | 15.799 | 37.966 | 65.154 | 1.00 | 36.43 | B |
| ATOM | 5094 | O | TYR | B | 187 | 15.601 | 38.276 | 66.323 | 1.00 | 36.43 | B |
| ATOM | 5095 | N | ASN | B | 188 | 16.935 | 38.221 | 64.514 | 1.00 | 37.49 | B |
| ATOM | 5096 | CA | ASN | B | 188 | 18.047 | 38.915 | 65.135 | 1.00 | 37.49 | B |
| ATOM | 5097 | CB | ASN | B | 188 | 19.342 | 38.494 | 64.452 | 1.00 | 178.91 | B |
| ATOM | 5098 | CG | ASN | B | 188 | 20.544 | 39.134 | 65.076 | 1.00 | 39.94 | B |
| ATOM | 5099 | OD1 | ASN | B | 188 | 20.607 | 40.343 | 65.217 | 1.00 | 39.94 | B |
| ATOM | 5100 | ND2 | ASN | B | 188 | 21.509 | 38.331 | 65.452 | 1.00 | 39.94 | B |
| ATOM | 5101 | C | ASN | B | 188 | 17.839 | 40.427 | 64.953 | 1.00 | 37.49 | B |
| ATOM | 5102 | O | ASN | B | 188 | 17.920 | 40.946 | 63.835 | 1.00 | 37.49 | B |
| ATOM | 5103 | N | HIS | B | 189 | 17.574 | 41.140 | 66.042 | 1.00 | 36.02 | B |
| ATOM | 5104 | CA | HIS | B | 189 | 17.363 | 42.589 | 65.965 | 1.00 | 36.02 | B |
| ATOM | 5105 | CB | HIS | B | 189 | 16.811 | 43.083 | 67.279 | 1.00 | 87.04 | B |
| ATOM | 5106 | CG | HIS | B | 189 | 15.397 | 42.675 | 67.496 | 1.00 | 47.58 | B |
| ATOM | 5107 | CD2 | HIS | B | 189 | 14.866 | 41.642 | 68.189 | 1.00 | 47.58 | B |
| ATOM | 5108 | ND1 | HIS | B | 189 | 14.337 | 43.309 | 66.885 | 1.00 | 47.58 | B |
| ATOM | 5109 | CE1 | HIS | B | 189 | 13.213 | 42.682 | 67.194 | 1.00 | 47.58 | B |
| ATOM | 5110 | NE2 | HIS | B | 189 | 13.506 | 41.665 | 67.984 | 1.00 | 47.58 | B |
| ATOM | 5111 | C | HIS | B | 189 | 18.584 | 43.424 | 65.570 | 1.00 | 36.02 | B |
| ATOM | 5112 | O | HIS | B | 189 | 18.437 | 44.496 | 64.998 | 1.00 | 36.02 | B |
| ATOM | 5113 | N | GLU | B | 190 | 19.784 | 42.928 | 65.866 | 1.00 | 44.32 | B |
| ATOM | 5114 | CA | GLU | B | 190 | 21.013 | 43.626 | 65.526 | 1.00 | 44.32 | B |
| ATOM | 5115 | CB | GLU | B | 190 | 22.182 | 42.954 | 66.229 | 1.00 | 104.31 | B |
| ATOM | 5116 | CG | GLU | B | 190 | 23.395 | 43.821 | 66.441 | 1.00 | 78.73 | B |
| ATOM | 5117 | CD | GLU | B | 190 | 24.470 | 43.096 | 67.245 | 1.00 | 78.73 | B |
| ATOM | 5118 | OE1 | GLU | B | 190 | 25.016 | 42.091 | 66.742 | 1.00 | 78.73 | B |
| ATOM | 5119 | OE2 | GLU | B | 190 | 24.764 | 43.517 | 68.384 | 1.00 | 78.73 | B |
| ATOM | 5120 | C | GLU | B | 190 | 21.185 | 43.537 | 64.010 | 1.00 | 44.32 | B |
| ATOM | 5121 | O | GLU | B | 190 | 21.304 | 44.556 | 63.325 | 1.00 | 44.32 | B |
| ATOM | 5122 | N | THR | B | 191 | 21.166 | 42.307 | 63.494 | 1.00 | 37.82 | B |
| ATOM | 5123 | CA | THR | B | 191 | 21.334 | 42.033 | 62.074 | 1.00 | 37.82 | B |
| ATOM | 5124 | CB | THR | B | 191 | 21.705 | 40.576 | 61.870 | 1.00 | 56.57 | B |
| ATOM | 5125 | OG1 | THR | B | 191 | 22.949 | 40.326 | 62.522 | 1.00 | 56.57 | B |
| ATOM | 5126 | CG2 | THR | B | 191 | 21.839 | 40.262 | 60.387 | 1.00 | 56.57 | B |
| ATOM | 5127 | C | THR | B | 191 | 20.132 | 42.340 | 61.192 | 1.00 | 37.82 | B |
| ATOM | 5128 | O | THR | B | 191 | 20.300 | 42.652 | 60.025 | 1.00 | 37.82 | B |
| ATOM | 5129 | N | ARG | B | 192 | 18.929 | 42.219 | 61.744 | 1.00 | 59.03 | B |
| ATOM | 5130 | CA | ARG | B | 192 | 17.694 | 42.476 | 61.008 | 1.00 | 59.03 | B |
| ATOM | 5131 | CB | ARG | B | 192 | 17.793 | 43.790 | 60.231 | 1.00 | 87.90 | B |
| ATOM | 5132 | CG | ARG | B | 192 | 17.883 | 45.007 | 61.117 | 1.00 | 87.90 | B |
| ATOM | 5133 | CD | ARG | B | 192 | 16.690 | 45.071 | 62.054 | 1.00 | 87.90 | B |
| ATOM | 5134 | NE | ARG | B | 192 | 16.836 | 46.105 | 63.077 | 1.00 | 87.90 | B |
| ATOM | 5135 | CZ | ARG | B | 192 | 16.893 | 47.408 | 62.824 | 1.00 | 87.90 | B |
| ATOM | 5136 | NH1 | ARG | B | 192 | 16.814 | 47.842 | 61.573 | 1.00 | 87.90 | B |
| ATOM | 5137 | NH2 | ARG | B | 192 | 17.026 | 48.275 | 63.822 | 1.00 | 87.90 | B |
| ATOM | 5138 | C | ARG | B | 192 | 17.273 | 41.349 | 60.070 | 1.00 | 59.03 | B |
| ATOM | 5139 | O | ARG | B | 192 | 16.642 | 41.596 | 59.045 | 1.00 | 59.03 | B |
| ATOM | 5140 | N | HIS | B | 193 | 17.624 | 40.115 | 60.419 | 1.00 | 47.20 | B |
| ATOM | 5141 | CA | HIS | B | 193 | 17.236 | 38.951 | 59.633 | 1.00 | 47.20 | B |
| ATOM | 5142 | CB | HIS | B | 193 | 18.179 | 38.768 | 58.443 | 1.00 | 68.35 | B |
| ATOM | 5143 | CG | HIS | B | 193 | 19.540 | 38.268 | 58.806 | 1.00 | 68.35 | B |
| ATOM | 5144 | CD2 | HIS | B | 193 | 20.000 | 37.629 | 59.905 | 1.00 | 68.35 | B |
| ATOM | 5145 | ND1 | HIS | B | 193 | 20.605 | 38.346 | 57.939 | 1.00 | 68.35 | B |
| ATOM | 5146 | CE1 | HIS | B | 193 | 21.663 | 37.774 | 58.485 | 1.00 | 68.35 | B |
| ATOM | 5147 | NE2 | HIS | B | 193 | 21.322 | 37.331 | 59.679 | 1.00 | 68.35 | B |
| ATOM | 5148 | C | HIS | B | 193 | 17.239 | 37.737 | 60.570 | 1.00 | 47.20 | B |
| ATOM | 5149 | O | HIS | B | 193 | 17.708 | 37.834 | 61.704 | 1.00 | 47.20 | B |
| ATOM | 5150 | N | GLY | B | 194 | 16.715 | 36.603 | 60.120 | 1.00 | 49.06 | B |
| ATOM | 5151 | CA | GLY | B | 194 | 16.657 | 35.443 | 60.996 | 1.00 | 49.06 | B |
| ATOM | 5152 | C | GLY | B | 194 | 17.948 | 34.673 | 61.135 | 1.00 | 49.06 | B |
| ATOM | 5153 | O | GLY | B | 194 | 18.846 | 34.830 | 60.320 | 1.00 | 49.06 | B |
| ATOM | 5154 | N | ASN | B | 195 | 18.044 | 33.849 | 62.178 | 1.00 | 45.65 | B |
| ATOM | 5155 | CA | ASN | B | 195 | 19.231 | 33.023 | 62.426 | 1.00 | 45.65 | B |
| ATOM | 5156 | CB | ASN | B | 195 | 20.112 | 33.587 | 63.545 | 1.00 | 57.59 | B |
| ATOM | 5157 | CG | ASN | B | 195 | 20.809 | 34.856 | 63.159 | 1.00 | 57.59 | B |
| ATOM | 5158 | OD1 | ASN | B | 195 | 21.341 | 34.983 | 62.055 | 1.00 | 57.59 | B |
| ATOM | 5159 | ND2 | ASN | B | 195 | 20.832 | 35.804 | 64.076 | 1.00 | 57.59 | B |
| ATOM | 5160 | C | ASN | B | 195 | 18.762 | 31.660 | 62.880 | 1.00 | 45.65 | B |
| ATOM | 5161 | O | ASN | B | 195 | 17.801 | 31.565 | 63.651 | 1.00 | 45.65 | B |
| ATOM | 5162 | N | VAL | B | 196 | 19.430 | 30.606 | 62.413 | 1.00 | 39.21 | B |
| ATOM | 5163 | CA | VAL | B | 196 | 19.052 | 29.270 | 62.836 | 1.00 | 39.21 | B |
| ATOM | 5164 | CB | VAL | B | 196 | 19.555 | 28.199 | 61.875 | 1.00 | 34.11 | B |
| ATOM | 5165 | CG1 | VAL | B | 196 | 19.177 | 26.817 | 62.405 | 1.00 | 34.11 | B |
| ATOM | 5166 | CG2 | VAL | B | 196 | 18.952 | 28.419 | 60.510 | 1.00 | 34.11 | B |
| ATOM | 5167 | C | VAL | B | 196 | 19.699 | 29.050 | 64.185 | 1.00 | 39.21 | B |
| ATOM | 5168 | O | VAL | B | 196 | 20.918 | 29.077 | 64.291 | 1.00 | 39.21 | B |
| ATOM | 5169 | N | ASN | B | 197 | 18.884 | 28.848 | 65.213 | 1.00 | 55.18 | B |
| ATOM | 5170 | CA | ASN | B | 197 | 19.406 | 28.634 | 66.554 | 1.00 | 55.18 | B |
| ATOM | 5171 | CB | ASN | B | 197 | 18.457 | 29.202 | 67.612 | 1.00 | 62.15 | B |
| ATOM | 5172 | CG | ASN | B | 197 | 17.999 | 30.592 | 67.294 | 1.00 | 62.15 | B |
| ATOM | 5173 | OD1 | ASN | B | 197 | 18.801 | 31.457 | 66.960 | 1.00 | 62.15 | B |
| ATOM | 5174 | ND2 | ASN | B | 197 | 16.697 | 30.822 | 67.404 | 1.00 | 62.15 | B |
| ATOM | 5175 | C | ASN | B | 197 | 19.580 | 27.152 | 66.830 | 1.00 | 55.18 | B |
| ATOM | 5176 | O | ASN | B | 197 | 20.469 | 26.753 | 67.579 | 1.00 | 55.18 | B |
| ATOM | 5177 | N | PHE | B | 198 | 18.726 | 26.336 | 66.228 | 1.00 | 35.44 | B |
| ATOM | 5178 | CA | PHE | B | 198 | 18.776 | 24.907 | 66.452 | 1.00 | 35.44 | B |
| ATOM | 5179 | CB | PHE | B | 198 | 17.722 | 24.522 | 67.490 | 1.00 | 33.11 | B |
| ATOM | 5180 | CG | PHE | B | 198 | 17.714 | 23.074 | 67.828 | 1.00 | 33.11 | B |
| ATOM | 5181 | CD1 | PHE | B | 198 | 18.484 | 22.589 | 68.875 | 1.00 | 33.11 | B |
| ATOM | 5182 | CD2 | PHE | B | 198 | 16.994 | 22.175 | 67.051 | 1.00 | 33.11 | B |
| ATOM | 5183 | CE1 | PHE | B | 198 | 18.548 | 21.216 | 69.145 | 1.00 | 33.11 | B |
| ATOM | 5184 | CE2 | PHE | B | 198 | 17.048 | 20.801 | 67.310 | 1.00 | 33.11 | B |
| ATOM | 5185 | CZ | PHE | B | 198 | 17.831 | 20.321 | 68.361 | 1.00 | 33.11 | B |
| ATOM | 5186 | C | PHE | B | 198 | 18.530 | 24.110 | 65.186 | 1.00 | 35.44 | B |
| ATOM | 5187 | O | PHE | B | 198 | 17.748 | 24.506 | 64.325 | 1.00 | 35.44 | B |
| ATOM | 5188 | N | ARG | B | 199 | 19.203 | 22.979 | 65.079 | 1.00 | 54.29 | B |
| ATOM | 5189 | CA | ARG | B | 199 | 19.027 | 22.105 | 63.939 | 1.00 | 54.29 | B |
| ATOM | 5190 | CB | ARG | B | 199 | 19.997 | 22.443 | 62.819 | 1.00 | 54.30 | B |
| ATOM | 5191 | CG | ARG | B | 199 | 19.840 | 21.495 | 61.658 | 1.00 | 54.30 | B |
| ATOM | 5192 | CD | ARG | B | 199 | 18.590 | 21.815 | 60.864 | 1.00 | 54.30 | B |
| ATOM | 5193 | NE | ARG | B | 199 | 18.933 | 22.633 | 59.706 | 1.00 | 54.30 | B |
| ATOM | 5194 | CZ | ARG | B | 199 | 18.183 | 23.618 | 59.232 | 1.00 | 54.30 | B |
| ATOM | 5195 | NH1 | ARG | B | 199 | 17.042 | 23.919 | 59.814 | 1.00 | 54.30 | B |
| ATOM | 5196 | NH2 | ARG | B | 199 | 18.578 | 24.307 | 58.174 | 1.00 | 54.30 | B |
| ATOM | 5197 | C | ARG | B | 199 | 19.291 | 20.687 | 64.404 | 1.00 | 54.29 | B |
| ATOM | 5198 | O | ARG | B | 199 | 20.391 | 20.369 | 64.852 | 1.00 | 54.29 | B |
| ATOM | 5199 | N | ALA | B | 200 | 18.285 | 19.831 | 64.309 | 1.00 | 41.53 | B |
| ATOM | 5200 | CA | ALA | B | 200 | 18.448 | 18.447 | 64.730 | 1.00 | 41.53 | B |
| ATOM | 5201 | CB | ALA | B | 200 | 17.162 | 17.681 | 64.493 | 1.00 | 44.85 | B |
| ATOM | 5202 | C | ALA | B | 200 | 19.605 | 17.786 | 63.983 | 1.00 | 41.53 | B |
| ATOM | 5203 | O | ALA | B | 200 | 19.748 | 17.941 | 62.769 | 1.00 | 41.53 | B |
| ATOM | 5204 | N | ASP | B | 201 | 20.426 | 17.045 | 64.718 | 1.00 | 58.31 | B |
| ATOM | 5205 | CA | ASP | B | 201 | 21.585 | 16.369 | 64.149 | 1.00 | 58.31 | B |
| ATOM | 5206 | CB | ASP | B | 201 | 22.345 | 15.657 | 65.258 | 1.00 | 134.34 | B |
| ATOM | 5207 | CG | ASP | B | 201 | 21.433 | 14.820 | 66.121 | 1.00 | 134.34 | B |
| ATOM | 5208 | OD1 | ASP | B | 201 | 20.592 | 15.412 | 66.836 | 1.00 | 134.34 | B |
| ATOM | 5209 | OD2 | ASP | B | 201 | 21.546 | 13.575 | 66.073 | 1.00 | 134.34 | B |
| ATOM | 5210 | C | ASP | B | 201 | 21.244 | 15.363 | 63.053 | 1.00 | 58.31 | B |
| ATOM | 5211 | O | ASP | B | 201 | 22.054 | 15.120 | 62.157 | 1.00 | 58.31 | B |
| ATOM | 5212 | N | TRP | B | 202 | 20.054 | 14.775 | 63.115 | 1.00 | 64.17 | B |
| ATOM | 5213 | CA | TRP | B | 202 | 19.656 | 13.778 | 62.123 | 1.00 | 64.17 | B |
| ATOM | 5214 | CB | TRP | B | 202 | 18.683 | 12.776 | 62.733 | 1.00 | 42.54 | B |
| ATOM | 5215 | CG | TRP | B | 202 | 17.481 | 13.384 | 63.378 | 1.00 | 42.54 | B |
| ATOM | 5216 | CD2 | TRP | B | 202 | 16.229 | 13.696 | 62.748 | 1.00 | 42.54 | B |
| ATOM | 5217 | CE2 | TRP | B | 202 | 15.372 | 14.209 | 63.751 | 1.00 | 42.54 | B |
| ATOM | 5218 | CE3 | TRP | B | 202 | 15.746 | 13.589 | 61.436 | 1.00 | 42.54 | B |
| ATOM | 5219 | CD1 | TRP | B | 202 | 17.337 | 13.718 | 64.696 | 1.00 | 42.54 | B |
| ATOM | 5220 | NE1 | TRP | B | 202 | 16.072 | 14.211 | 64.929 | 1.00 | 42.54 | B |
| ATOM | 5221 | CZ2 | TRP | B | 202 | 14.060 | 14.612 | 63.480 | 1.00 | 42.54 | B |
| ATOM | 5222 | CZ3 | TRP | B | 202 | 14.442 | 13.989 | 61.169 | 1.00 | 42.54 | B |
| ATOM | 5223 | CH2 | TRP | B | 202 | 13.616 | 14.494 | 62.189 | 1.00 | 42.54 | B |
| ATOM | 5224 | C | TRP | B | 202 | 19.037 | 14.302 | 60.837 | 1.00 | 64.17 | B |
| ATOM | 5225 | O | TRP | B | 202 | 18.819 | 13.526 | 59.901 | 1.00 | 64.17 | B |
| ATOM | 5226 | N | PHE | B | 203 | 18.764 | 15.604 | 60.782 | 1.00 | 47.65 | B |
| ATOM | 5227 | CA | PHE | B | 203 | 18.136 | 16.200 | 59.614 | 1.00 | 47.65 | B |
| ATOM | 5228 | CB | PHE | B | 203 | 17.072 | 17.193 | 60.078 | 1.00 | 39.15 | B |
| ATOM | 5229 | CG | PHE | B | 203 | 16.210 | 17.724 | 58.971 | 1.00 | 39.15 | B |
| ATOM | 5230 | CD1 | PHE | B | 203 | 15.297 | 16.896 | 58.324 | 1.00 | 39.15 | B |
| ATOM | 5231 | CD2 | PHE | B | 203 | 16.303 | 19.054 | 58.573 | 1.00 | 39.15 | B |
| ATOM | 5232 | CE1 | PHE | B | 203 | 14.484 | 17.394 | 57.290 | 1.00 | 39.15 | B |
| ATOM | 5233 | CE2 | PHE | B | 203 | 15.504 | 19.555 | 57.552 | 1.00 | 39.15 | B |
| ATOM | 5234 | CZ | PHE | B | 203 | 14.594 | 18.729 | 56.910 | 1.00 | 39.15 | B |
| ATOM | 5235 | C | PHE | B | 203 | 19.116 | 16.897 | 58.689 | 1.00 | 47.65 | B |
| ATOM | 5236 | O | PHE | B | 203 | 19.641 | 17.955 | 59.032 | 1.00 | 47.65 | B |
| ATOM | 5237 | N | ASN | B | 204 | 19.376 | 16.293 | 57.528 | 1.00 | 50.31 | B |
| ATOM | 5238 | CA | ASN | B | 204 | 20.258 | 16.895 | 56.531 | 1.00 | 50.31 | B |
| ATOM | 5239 | CB | ASN | B | 204 | 21.065 | 15.834 | 55.758 | 1.00 | 90.15 | B |
| ATOM | 5240 | CG | ASN | B | 204 | 21.770 | 16.409 | 54.528 | 1.00 | 90.15 | B |
| ATOM | 5241 | OD1 | ASN | B | 204 | 21.116 | 16.891 | 53.611 | 1.00 | 90.15 | B |
| ATOM | 5242 | ND2 | ASN | B | 204 | 23.096 | 16.363 | 54.495 | 1.00 | 90.15 | B |
| ATOM | 5243 | C | ASN | B | 204 | 19.295 | 17.617 | 55.598 | 1.00 | 50.31 | B |
| ATOM | 5244 | O | ASN | B | 204 | 18.685 | 16.995 | 54.736 | 1.00 | 50.31 | B |
| ATOM | 5245 | N | ILE | B | 205 | 19.152 | 18.928 | 55.780 | 1.00 | 57.35 | B |
| ATOM | 5246 | CA | ILE | B | 205 | 18.227 | 19.707 | 54.969 | 1.00 | 57.35 | B |
| ATOM | 5247 | CB | ILE | B | 205 | 18.246 | 21.200 | 55.377 | 1.00 | 56.24 | B |
| ATOM | 5248 | CG2 | ILE | B | 205 | 19.411 | 21.914 | 54.735 | 1.00 | 56.24 | B |
| ATOM | 5249 | CG1 | ILE | B | 205 | 16.936 | 21.864 | 54.954 | 1.00 | 56.24 | B |
| ATOM | 5250 | CD1 | ILE | B | 205 | 16.657 | 23.158 | 55.685 | 1.00 | 56.24 | B |
| ATOM | 5251 | C | ILE | B | 205 | 18.454 | 19.589 | 53.466 | 1.00 | 57.35 | B |
| ATOM | 5252 | O | ILE | B | 205 | 17.564 | 19.900 | 52.680 | 1.00 | 57.35 | B |
| ATOM | 5253 | N | SER | B | 206 | 19.633 | 19.133 | 53.063 | 1.00 | 49.65 | B |
| ATOM | 5254 | CA | SER | B | 206 | 19.921 | 18.993 | 51.642 | 1.00 | 49.65 | B |
| ATOM | 5255 | CB | SER | B | 206 | 21.418 | 18.836 | 51.398 | 1.00 | 52.52 | B |
| ATOM | 5256 | OG | SER | B | 206 | 22.095 | 20.031 | 51.716 | 1.00 | 52.52 | B |
| ATOM | 5257 | C | SER | B | 206 | 19.201 | 17.788 | 51.081 | 1.00 | 49.65 | B |
| ATOM | 5258 | O | SER | B | 206 | 18.993 | 17.691 | 49.870 | 1.00 | 49.65 | B |
| ATOM | 5259 | N | ARG | B | 207 | 18.825 | 16.867 | 51.961 | 1.00 | 49.08 | B |
| ATOM | 5260 | CA | ARG | B | 207 | 18.124 | 15.673 | 51.535 | 1.00 | 49.08 | B |
| ATOM | 5261 | CB | ARG | B | 207 | 18.536 | 14.474 | 52.389 | 1.00 | 69.41 | B |
| ATOM | 5262 | CG | ARG | B | 207 | 19.905 | 13.945 | 52.046 | 1.00 | 69.41 | B |
| ATOM | 5263 | CD | ARG | B | 207 | 19.996 | 12.458 | 52.299 | 1.00 | 69.41 | B |
| ATOM | 5264 | NE | ARG | B | 207 | 20.131 | 12.122 | 53.715 | 1.00 | 69.41 | B |
| ATOM | 5265 | CZ | ARG | B | 207 | 19.742 | 10.962 | 54.246 | 1.00 | 69.41 | B |
| ATOM | 5266 | NH1 | ARG | B | 207 | 19.187 | 10.034 | 53.473 | 1.00 | 69.41 | B |
| ATOM | 5267 | NH2 | ARG | B | 207 | 19.913 | 10.725 | 55.546 | 1.00 | 69.41 | B |
| ATOM | 5268 | C | ARG | B | 207 | 16.616 | 15.862 | 51.594 | 1.00 | 49.08 | B |
| ATOM | 5269 | O | ARG | B | 207 | 15.868 | 14.906 | 51.433 | 1.00 | 49.08 | B |
| ATOM | 5270 | N | SER | B | 208 | 16.168 | 17.095 | 51.831 | 1.00 | 46.55 | B |
| ATOM | 5271 | CA | SER | B | 208 | 14.739 | 17.382 | 51.901 | 1.00 | 46.55 | B |
| ATOM | 5272 | CB | SER | B | 208 | 14.334 | 17.738 | 53.320 | 1.00 | 44.08 | B |
| ATOM | 5273 | OG | SER | B | 208 | 12.932 | 17.918 | 53.392 | 1.00 | 44.08 | B |
| ATOM | 5274 | C | SER | B | 208 | 14.372 | 18.523 | 50.963 | 1.00 | 46.55 | B |
| ATOM | 5275 | O | SER | B | 208 | 14.050 | 19.632 | 51.392 | 1.00 | 46.55 | B |
| ATOM | 5276 | N | PRO | B | 209 | 14.406 | 18.249 | 49.654 | 1.00 | 38.87 | B |
| ATOM | 5277 | CD | PRO | B | 209 | 14.657 | 16.892 | 49.138 | 1.00 | 47.88 | B |
| ATOM | 5278 | CA | PRO | B | 209 | 14.105 | 19.168 | 48.552 | 1.00 | 38.87 | B |
| ATOM | 5279 | CB | PRO | B | 209 | 13.992 | 18.235 | 47.350 | 1.00 | 47.88 | B |
| ATOM | 5280 | CG | PRO | B | 209 | 14.952 | 17.161 | 47.678 | 1.00 | 47.88 | B |
| ATOM | 5281 | C | PRO | B | 209 | 12.838 | 19.979 | 48.729 | 1.00 | 38.87 | B |
| ATOM | 5282 | O | PRO | B | 209 | 12.723 | 21.068 | 48.197 | 1.00 | 38.87 | B |
| ATOM | 5283 | N | HIS | B | 210 | 11.886 | 19.439 | 49.474 | 1.00 | 40.48 | B |
| ATOM | 5284 | CA | HIS | B | 210 | 10.623 | 20.115 | 49.649 | 1.00 | 40.48 | B |
| ATOM | 5285 | CB | HIS | B | 210 | 9.496 | 19.107 | 49.480 | 1.00 | 45.25 | B |
| ATOM | 5286 | CG | HIS | B | 210 | 9.448 | 18.506 | 48.112 | 1.00 | 45.25 | B |
| ATOM | 5287 | CD2 | HIS | B | 210 | 9.794 | 17.274 | 47.668 | 1.00 | 45.25 | B |
| ATOM | 5288 | ND1 | HIS | B | 210 | 9.065 | 19.224 | 46.998 | 1.00 | 45.25 | B |
| ATOM | 5289 | CE1 | HIS | B | 210 | 9.178 | 18.461 | 45.927 | 1.00 | 45.25 | B |
| ATOM | 5290 | NE2 | HIS | B | 210 | 9.620 | 17.274 | 46.306 | 1.00 | 45.25 | B |
| ATOM | 5291 | C | HIS | B | 210 | 10.462 | 20.927 | 50.927 | 1.00 | 40.48 | B |
| ATOM | 5292 | O | HIS | B | 210 | 9.369 | 21.426 | 51.232 | 1.00 | 40.48 | B |
| ATOM | 5293 | N | THR | B | 211 | 11.546 | 21.062 | 51.680 | 1.00 | 41.33 | B |
| ATOM | 5294 | CA | THR | B | 211 | 11.512 | 21.898 | 52.865 | 1.00 | 41.33 | B |
| ATOM | 5295 | CB | THR | B | 211 | 12.577 | 21.494 | 53.870 | 1.00 | 59.75 | B |
| ATOM | 5296 | OG1 | THR | B | 211 | 12.329 | 20.157 | 54.318 | 1.00 | 59.75 | B |
| ATOM | 5297 | CG2 | THR | B | 211 | 12.547 | 22.429 | 55.060 | 1.00 | 59.75 | B |
| ATOM | 5298 | C | THR | B | 211 | 11.866 | 23.267 | 52.280 | 1.00 | 41.33 | B |
| ATOM | 5299 | O | THR | B | 211 | 12.883 | 23.406 | 51.596 | 1.00 | 41.33 | B |
| ATOM | 5300 | N | PRO | B | 212 | 11.031 | 24.289 | 52.518 | 1.00 | 40.60 | B |
| ATOM | 5301 | CD | PRO | B | 212 | 9.869 | 24.251 | 53.417 | 1.00 | 41.99 | B |
| ATOM | 5302 | CA | PRO | B | 212 | 11.253 | 25.646 | 52.001 | 1.00 | 40.60 | B |
| ATOM | 5303 | CB | PRO | B | 212 | 10.249 | 26.474 | 52.786 | 1.00 | 41.99 | B |
| ATOM | 5304 | CG | PRO | B | 212 | 9.135 | 25.491 | 53.018 | 1.00 | 41.99 | B |
| ATOM | 5305 | C | PRO | B | 212 | 12.683 | 26.185 | 52.135 | 1.00 | 40.60 | B |
| ATOM | 5306 | O | PRO | B | 212 | 13.347 | 25.954 | 53.142 | 1.00 | 40.60 | B |
| ATOM | 5307 | N | GLY | B | 213 | 13.151 | 26.907 | 51.119 | 1.00 | 45.88 | B |
| ATOM | 5308 | CA | GLY | B | 213 | 14.492 | 27.469 | 51.153 | 1.00 | 45.88 | B |
| ATOM | 5309 | C | GLY | B | 213 | 14.535 | 28.813 | 51.849 | 1.00 | 45.88 | B |
| ATOM | 5310 | O | GLY | B | 213 | 13.502 | 29.298 | 52.310 | 1.00 | 45.88 | B |
| ATOM | 5311 | N | ASN | B | 214 | 15.715 | 29.428 | 51.917 | 1.00 | 56.30 | B |
| ATOM | 5312 | CA | ASN | B | 214 | 15.903 | 30.720 | 52.591 | 1.00 | 56.30 | B |
| ATOM | 5313 | CB | ASN | B | 214 | 15.076 | 31.827 | 51.952 | 1.00 | 82.78 | B |
| ATOM | 5314 | CG | ASN | B | 214 | 15.023 | 31.722 | 50.466 | 1.00 | 76.95 | B |
| ATOM | 5315 | OD1 | ASN | B | 214 | 15.838 | 31.044 | 49.853 | 1.00 | 76.95 | B |
| ATOM | 5316 | ND2 | ASN | B | 214 | 14.059 | 32.406 | 49.864 | 1.00 | 76.95 | B |
| ATOM | 5317 | C | ASN | B | 214 | 15.434 | 30.566 | 54.019 | 1.00 | 56.30 | B |
| ATOM | 5318 | O | ASN | B | 214 | 14.613 | 31.352 | 54.497 | 1.00 | 56.30 | B |
| ATOM | 5319 | N | ASP | B | 215 | 15.946 | 29.549 | 54.701 | 1.00 | 39.70 | B |
| ATOM | 5320 | CA | ASP | B | 215 | 15.550 | 29.289 | 56.074 | 1.00 | 39.70 | B |
| ATOM | 5321 | CB | ASP | B | 215 | 16.249 | 30.253 | 57.010 | 1.00 | 151.11 | B |
| ATOM | 5322 | CG | ASP | B | 215 | 17.738 | 30.206 | 56.852 | 1.00 | 72.49 | B |
| ATOM | 5323 | OD1 | ASP | B | 215 | 18.318 | 29.107 | 56.994 | 1.00 | 72.49 | B |
| ATOM | 5324 | OD2 | ASP | B | 215 | 18.333 | 31.266 | 56.578 | 1.00 | 72.49 | B |
| ATOM | 5325 | C | ASP | B | 215 | 14.046 | 29.425 | 56.198 | 1.00 | 39.70 | B |
| ATOM | 5326 | O | ASP | B | 215 | 13.557 | 30.311 | 56.889 | 1.00 | 39.70 | B |
| ATOM | 5327 | N | TYR | B | 216 | 13.330 | 28.557 | 55.488 | 1.00 | 32.61 | B |
| ATOM | 5328 | CA | TYR | B | 216 | 11.882 | 28.521 | 55.515 | 1.00 | 32.61 | B |
| ATOM | 5329 | CB | TYR | B | 216 | 11.390 | 28.029 | 56.881 | 1.00 | 56.70 | B |
| ATOM | 5330 | CG | TYR | B | 216 | 12.017 | 26.722 | 57.322 | 1.00 | 35.45 | B |
| ATOM | 5331 | CD1 | TYR | B | 216 | 13.366 | 26.657 | 57.655 | 1.00 | 35.45 | B |
| ATOM | 5332 | CE1 | TYR | B | 216 | 13.943 | 25.478 | 58.061 | 1.00 | 35.45 | B |
| ATOM | 5333 | CD2 | TYR | B | 216 | 11.258 | 25.551 | 57.412 | 1.00 | 35.45 | B |
| ATOM | 5334 | CE2 | TYR | B | 216 | 11.831 | 24.363 | 57.826 | 1.00 | 35.45 | B |
| ATOM | 5335 | CZ | TYR | B | 216 | 13.175 | 24.338 | 58.148 | 1.00 | 35.45 | B |
| ATOM | 5336 | OH | TYR | B | 216 | 13.763 | 23.175 | 58.566 | 1.00 | 35.45 | B |
| ATOM | 5337 | C | TYR | B | 216 | 11.223 | 29.853 | 55.195 | 1.00 | 32.61 | B |
| ATOM | 5338 | O | TYR | B | 216 | 10.159 | 30.168 | 55.748 | 1.00 | 32.61 | B |
| ATOM | 5339 | N | ASN | B | 217 | 11.832 | 30.621 | 54.292 | 1.00 | 34.55 | B |
| ATOM | 5340 | CA | ASN | B | 217 | 11.283 | 31.913 | 53.917 | 1.00 | 34.55 | B |
| ATOM | 5341 | CB | ASN | B | 217 | 9.980 | 31.735 | 53.132 | 1.00 | 39.38 | B |
| ATOM | 5342 | CG | ASN | B | 217 | 10.182 | 31.001 | 51.829 | 1.00 | 34.11 | B |
| ATOM | 5343 | OD1 | ASN | B | 217 | 9.521 | 29.999 | 51.576 | 1.00 | 34.11 | B |
| ATOM | 5344 | ND2 | ASN | B | 217 | 11.094 | 31.490 | 50.994 | 1.00 | 34.11 | B |
| ATOM | 5345 | C | ASN | B | 217 | 10.998 | 32.720 | 55.177 | 1.00 | 34.55 | B |
| ATOM | 5346 | O | ASN | B | 217 | 10.003 | 33.451 | 55.244 | 1.00 | 34.55 | B |
| ATOM | 5347 | N | PHE | B | 218 | 11.874 | 32.602 | 56.172 | 1.00 | 28.07 | B |
| ATOM | 5348 | CA | PHE | B | 218 | 11.657 | 33.298 | 57.428 | 1.00 | 28.07 | B |
| ATOM | 5349 | CB | PHE | B | 218 | 12.727 | 32.929 | 58.443 | 1.00 | 39.29 | B |
| ATOM | 5350 | CG | PHE | B | 218 | 12.500 | 33.549 | 59.782 | 1.00 | 35.81 | B |
| ATOM | 5351 | CD1 | PHE | B | 218 | 11.646 | 32.951 | 60.699 | 1.00 | 35.81 | B |
| ATOM | 5352 | CD2 | PHE | B | 218 | 13.098 | 34.760 | 60.107 | 1.00 | 35.81 | B |
| ATOM | 5353 | CE1 | PHE | B | 218 | 11.387 | 33.548 | 61.924 | 1.00 | 35.81 | B |
| ATOM | 5354 | CE2 | PHE | B | 218 | 12.847 | 35.368 | 61.323 | 1.00 | 35.81 | B |
| ATOM | 5355 | CZ | PHE | B | 218 | 11.986 | 34.758 | 62.239 | 1.00 | 35.81 | B |
| ATOM | 5356 | C | PHE | B | 218 | 11.594 | 34.819 | 57.325 | 1.00 | 28.07 | B |
| ATOM | 5357 | O | PHE | B | 218 | 10.656 | 35.444 | 57.830 | 1.00 | 28.07 | B |
| ATOM | 5358 | N | ASN | B | 219 | 12.594 | 35.418 | 56.693 | 1.00 | 37.00 | B |
| ATOM | 5359 | CA | ASN | B | 219 | 12.590 | 36.863 | 56.565 | 1.00 | 37.00 | B |
| ATOM | 5360 | CB | ASN | B | 219 | 13.786 | 37.351 | 55.753 | 1.00 | 40.42 | B |
| ATOM | 5361 | CG | ASN | B | 219 | 15.102 | 36.913 | 56.340 | 1.00 | 32.05 | B |
| ATOM | 5362 | OD1 | ASN | B | 219 | 15.266 | 36.854 | 57.558 | 1.00 | 32.05 | B |
| ATOM | 5363 | ND2 | ASN | B | 219 | 16.057 | 36.616 | 55.475 | 1.00 | 32.05 | B |
| ATOM | 5364 | C | ASN | B | 219 | 11.297 | 37.317 | 55.903 | 1.00 | 37.00 | B |
| ATOM | 5365 | O | ASN | B | 219 | 10.689 | 38.295 | 56.338 | 1.00 | 37.00 | B |
| ATOM | 5366 | N | PHE | B | 220 | 10.870 | 36.613 | 54.856 | 1.00 | 42.13 | B |
| ATOM | 5367 | CA | PHE | B | 220 | 9.630 | 36.961 | 54.172 | 1.00 | 42.13 | B |
| ATOM | 5368 | CB | PHE | B | 220 | 9.316 | 35.960 | 53.058 | 1.00 | 45.65 | B |
| ATOM | 5369 | CG | PHE | B | 220 | 7.908 | 36.061 | 52.536 | 1.00 | 38.63 | B |
| ATOM | 5370 | CD1 | PHE | B | 220 | 7.580 | 36.976 | 51.559 | 1.00 | 38.63 | B |
| ATOM | 5371 | CD2 | PHE | B | 220 | 6.889 | 35.270 | 53.059 | 1.00 | 38.63 | B |
| ATOM | 5372 | CE1 | PHE | B | 220 | 6.270 | 37.128 | 51.094 | 1.00 | 38.63 | B |
| ATOM | 5373 | CE2 | PHE | B | 220 | 5.566 | 35.412 | 52.603 | 1.00 | 38.63 | B |
| ATOM | 5374 | CZ | PHE | B | 220 | 5.269 | 36.354 | 51.612 | 1.00 | 38.63 | B |
| ATOM | 5375 | C | PHE | B | 220 | 8.460 | 36.979 | 55.146 | 1.00 | 42.13 | B |
| ATOM | 5376 | O | PHE | B | 220 | 7.774 | 37.988 | 55.283 | 1.00 | 42.13 | B |
| ATOM | 5377 | N | TRP | B | 221 | 8.234 | 35.856 | 55.819 | 1.00 | 39.06 | B |
| ATOM | 5378 | CA | TRP | B | 221 | 7.130 | 35.747 | 56.762 | 1.00 | 39.06 | B |
| ATOM | 5379 | CB | TRP | B | 221 | 6.994 | 34.299 | 57.246 | 1.00 | 42.05 | B |
| ATOM | 5380 | CG | TRP | B | 221 | 6.443 | 33.383 | 56.205 | 1.00 | 35.20 | B |
| ATOM | 5381 | CD2 | TRP | B | 221 | 5.156 | 33.480 | 55.578 | 1.00 | 35.20 | B |
| ATOM | 5382 | CE2 | TRP | B | 221 | 5.076 | 32.441 | 54.624 | 1.00 | 35.20 | B |
| ATOM | 5383 | CE3 | TRP | B | 221 | 4.061 | 34.346 | 55.731 | 1.00 | 35.20 | B |
| ATOM | 5384 | CD1 | TRP | B | 221 | 7.073 | 32.312 | 55.626 | 1.00 | 35.20 | B |
| ATOM | 5385 | NE1 | TRP | B | 221 | 6.256 | 31.744 | 54.676 | 1.00 | 35.20 | B |
| ATOM | 5386 | CZ2 | TRP | B | 221 | 3.947 | 32.245 | 53.823 | 1.00 | 35.20 | B |
| ATOM | 5387 | CZ3 | TRP | B | 221 | 2.936 | 34.149 | 54.933 | 1.00 | 35.20 | B |
| ATOM | 5388 | CH2 | TRP | B | 221 | 2.894 | 33.104 | 53.989 | 1.00 | 35.20 | B |
| ATOM | 5389 | C | TRP | B | 221 | 7.241 | 36.696 | 57.954 | 1.00 | 39.06 | B |
| ATOM | 5390 | O | TRP | B | 221 | 6.271 | 37.367 | 58.317 | 1.00 | 39.06 | B |
| ATOM | 5391 | N | TYR | B | 222 | 8.413 | 36.755 | 58.572 | 1.00 | 46.48 | B |
| ATOM | 5392 | CA | TYR | B | 222 | 8.574 | 37.643 | 59.710 | 1.00 | 46.48 | B |
| ATOM | 5393 | CB | TYR | B | 222 | 10.033 | 37.738 | 60.123 | 1.00 | 30.81 | B |
| ATOM | 5394 | CG | TYR | B | 222 | 10.243 | 38.824 | 61.141 | 1.00 | 29.38 | B |
| ATOM | 5395 | CD1 | TYR | B | 222 | 9.886 | 38.631 | 62.477 | 1.00 | 29.38 | B |
| ATOM | 5396 | CE1 | TYR | B | 222 | 10.021 | 39.652 | 63.402 | 1.00 | 29.38 | B |
| ATOM | 5397 | CD2 | TYR | B | 222 | 10.740 | 40.066 | 60.762 | 1.00 | 29.38 | B |
| ATOM | 5398 | CE2 | TYR | B | 222 | 10.877 | 41.094 | 61.674 | 1.00 | 29.38 | B |
| ATOM | 5399 | CZ | TYR | B | 222 | 10.513 | 40.884 | 62.991 | 1.00 | 29.38 | B |
| ATOM | 5400 | OH | TYR | B | 222 | 10.613 | 41.922 | 63.891 | 1.00 | 29.38 | B |
| ATOM | 5401 | C | TYR | B | 222 | 8.088 | 39.049 | 59.359 | 1.00 | 46.48 | B |
| ATOM | 5402 | O | TYR | B | 222 | 7.274 | 39.657 | 60.054 | 1.00 | 46.48 | B |
| ATOM | 5403 | N | SER | B | 223 | 8.614 | 39.558 | 58.263 | 1.00 | 34.83 | B |
| ATOM | 5404 | CA | SER | B | 223 | 8.264 | 40.877 | 57.812 | 1.00 | 34.83 | B |
| ATOM | 5405 | CB | SER | B | 223 | 9.092 | 41.206 | 56.596 | 1.00 | 35.31 | B |
| ATOM | 5406 | OG | SER | B | 223 | 8.658 | 42.435 | 56.067 | 1.00 | 33.93 | B |
| ATOM | 5407 | C | SER | B | 223 | 6.779 | 41.077 | 57.489 | 1.00 | 34.83 | B |
| ATOM | 5408 | O | SER | B | 223 | 6.156 | 42.043 | 57.933 | 1.00 | 34.83 | B |
| ATOM | 5409 | N | LEU | B | 224 | 6.215 | 40.164 | 56.705 | 1.00 | 41.87 | B |
| ATOM | 5410 | CA | LEU | B | 224 | 4.818 | 40.256 | 56.321 | 1.00 | 41.87 | B |
| ATOM | 5411 | CB | LEU | B | 224 | 4.505 | 39.231 | 55.241 | 1.00 | 58.24 | B |
| ATOM | 5412 | CG | LEU | B | 224 | 3.060 | 39.279 | 54.755 | 1.00 | 35.48 | B |
| ATOM | 5413 | CD1 | LEU | B | 224 | 2.677 | 40.680 | 54.321 | 1.00 | 35.48 | B |
| ATOM | 5414 | CD2 | LEU | B | 224 | 2.906 | 38.315 | 53.613 | 1.00 | 35.48 | B |
| ATOM | 5415 | C | LEU | B | 224 | 3.890 | 40.062 | 57.510 | 1.00 | 41.87 | B |
| ATOM | 5416 | O | LEU | B | 224 | 2.810 | 40.649 | 57.558 | 1.00 | 41.87 | B |
| ATOM | 5417 | N | MET | B | 225 | 4.306 | 39.238 | 58.469 | 1.00 | 51.73 | B |
| ATOM | 5418 | CA | MET | B | 225 | 3.489 | 39.001 | 59.645 | 1.00 | 51.73 | B |
| ATOM | 5419 | CB | MET | B | 225 | 3.949 | 37.750 | 60.370 | 1.00 | 73.83 | B |
| ATOM | 5420 | CG | MET | B | 225 | 3.675 | 36.501 | 59.588 | 1.00 | 42.55 | B |
| ATOM | 5421 | SD | MET | B | 225 | 4.035 | 35.041 | 60.525 | 1.00 | 42.55 | B |
| ATOM | 5422 | CE | MET | B | 225 | 2.839 | 35.210 | 61.865 | 1.00 | 42.55 | B |
| ATOM | 5423 | C | MET | B | 225 | 3.588 | 40.198 | 60.561 | 1.00 | 51.73 | B |
| ATOM | 5424 | O | MET | B | 225 | 2.629 | 40.556 | 61.238 | 1.00 | 51.73 | B |
| ATOM | 5425 | N | LYS | B | 226 | 4.759 | 40.822 | 60.582 | 1.00 | 51.23 | B |
| ATOM | 5426 | CA | LYS | B | 226 | 4.959 | 41.995 | 61.410 | 1.00 | 51.23 | B |
| ATOM | 5427 | CB | LYS | B | 226 | 6.396 | 42.493 | 61.292 | 1.00 | 95.84 | B |
| ATOM | 5428 | CG | LYS | B | 226 | 6.683 | 43.734 | 62.112 | 1.00 | 47.30 | B |
| ATOM | 5429 | CD | LYS | B | 226 | 8.066 | 44.241 | 61.811 | 1.00 | 47.30 | B |
| ATOM | 5430 | CE | LYS | B | 226 | 8.271 | 45.595 | 62.407 | 1.00 | 47.30 | B |
| ATOM | 5431 | NZ | LYS | B | 226 | 9.618 | 46.118 | 62.042 | 1.00 | 47.30 | B |
| ATOM | 5432 | C | LYS | B | 226 | 3.994 | 43.048 | 60.897 | 1.00 | 51.23 | B |
| ATOM | 5433 | O | LYS | B | 226 | 3.301 | 43.696 | 61.677 | 1.00 | 51.23 | B |
| ATOM | 5434 | N | GLU | B | 227 | 3.940 | 43.195 | 59.575 | 1.00 | 49.99 | B |
| ATOM | 5435 | CA | GLU | B | 227 | 3.045 | 44.159 | 58.936 | 1.00 | 49.99 | B |
| ATOM | 5436 | CB | GLU | B | 227 | 3.185 | 44.094 | 57.421 | 1.00 | 125.36 | B |
| ATOM | 5437 | CG | GLU | B | 227 | 2.381 | 45.156 | 56.707 | 1.00 | 101.16 | B |
| ATOM | 5438 | CD | GLU | B | 227 | 2.238 | 44.893 | 55.221 | 1.00 | 101.16 | B |
| ATOM | 5439 | OE1 | GLU | B | 227 | 3.270 | 44.679 | 54.545 | 1.00 | 101.16 | B |
| ATOM | 5440 | OE2 | GLU | B | 227 | 1.088 | 44.907 | 54.726 | 1.00 | 101.16 | B |
| ATOM | 5441 | C | GLU | B | 227 | 1.602 | 43.848 | 59.317 | 1.00 | 49.99 | B |
| ATOM | 5442 | O | GLU | B | 227 | 0.850 | 44.722 | 59.729 | 1.00 | 49.99 | B |
| ATOM | 5443 | N | THR | B | 228 | 1.223 | 42.585 | 59.185 | 1.00 | 47.68 | B |
| ATOM | 5444 | CA | THR | B | 228 | −0.127 | 42.157 | 59.518 | 1.00 | 47.68 | B |
| ATOM | 5445 | CB | THR | B | 228 | −0.314 | 40.657 | 59.250 | 1.00 | 53.19 | B |
| ATOM | 5446 | OG1 | THR | B | 228 | −0.017 | 40.374 | 57.882 | 1.00 | 39.18 | B |
| ATOM | 5447 | CG2 | THR | B | 228 | −1.740 | 40.239 | 59.540 | 1.00 | 39.18 | B |
| ATOM | 5448 | C | THR | B | 228 | −0.482 | 42.413 | 60.974 | 1.00 | 47.68 | B |
| ATOM | 5449 | O | THR | B | 228 | −1.601 | 42.787 | 61.277 | 1.00 | 47.68 | B |
| ATOM | 5450 | N | LEU | B | 229 | 0.462 | 42.195 | 61.877 | 1.00 | 54.06 | B |
| ATOM | 5451 | CA | LEU | B | 229 | 0.184 | 42.397 | 63.283 | 1.00 | 54.06 | B |
| ATOM | 5452 | CB | LEU | B | 229 | 1.331 | 41.861 | 64.135 | 1.00 | 67.78 | B |
| ATOM | 5453 | CG | LEU | B | 229 | 1.154 | 41.961 | 65.654 | 1.00 | 39.66 | B |
| ATOM | 5454 | CD1 | LEU | B | 229 | −0.251 | 41.538 | 66.079 | 1.00 | 39.66 | B |
| ATOM | 5455 | CD2 | LEU | B | 229 | 2.205 | 41.094 | 66.324 | 1.00 | 39.66 | B |
| ATOM | 5456 | C | LEU | B | 229 | −0.050 | 43.870 | 63.569 | 1.00 | 54.06 | B |
| ATOM | 5457 | O | LEU | B | 229 | −0.925 | 44.218 | 64.361 | 1.00 | 54.06 | B |
| ATOM | 5458 | N | GLU | B | 230 | 0.719 | 44.738 | 62.920 | 1.00 | 63.43 | B |
| ATOM | 5459 | CA | GLU | B | 230 | 0.560 | 46.179 | 63.113 | 1.00 | 63.43 | B |
| ATOM | 5460 | CB | GLU | B | 230 | 1.644 | 46.938 | 62.355 | 1.00 | 135.98 | B |
| ATOM | 5461 | CG | GLU | B | 230 | 3.027 | 46.762 | 62.958 | 1.00 | 100.44 | B |
| ATOM | 5462 | CD | GLU | B | 230 | 4.093 | 47.547 | 62.223 | 1.00 | 100.44 | B |
| ATOM | 5463 | OE1 | GLU | B | 230 | 3.888 | 48.758 | 62.013 | 1.00 | 100.44 | B |
| ATOM | 5464 | OE2 | GLU | B | 230 | 5.138 | 46.962 | 61.862 | 1.00 | 100.44 | B |
| ATOM | 5465 | C | GLU | B | 230 | −0.824 | 46.642 | 62.663 | 1.00 | 63.43 | B |
| ATOM | 5466 | O | GLU | B | 230 | −1.495 | 47.373 | 63.367 | 1.00 | 63.43 | B |
| ATOM | 5467 | N | GLU | B | 231 | −1.256 | 46.202 | 61.494 | 1.00 | 59.76 | B |
| ATOM | 5468 | CA | GLU | B | 231 | −2.570 | 46.568 | 60.979 | 1.00 | 59.76 | B |
| ATOM | 5469 | CB | GLU | B | 231 | −2.780 | 45.957 | 59.594 | 1.00 | 145.72 | B |
| ATOM | 5470 | CG | GLU | B | 231 | −1.697 | 46.302 | 58.592 | 1.00 | 121.96 | B |
| ATOM | 5471 | CD | GLU | B | 231 | −1.688 | 47.768 | 58.219 | 1.00 | 121.96 | B |
| ATOM | 5472 | OE1 | GLU | B | 231 | −1.608 | 48.617 | 59.132 | 1.00 | 121.96 | B |
| ATOM | 5473 | OE2 | GLU | B | 231 | −1.756 | 48.069 | 57.008 | 1.00 | 121.96 | B |
| ATOM | 5474 | C | GLU | B | 231 | −3.661 | 46.062 | 61.923 | 1.00 | 59.76 | B |
| ATOM | 5475 | O | GLU | B | 231 | −4.611 | 46.778 | 62.239 | 1.00 | 59.76 | B |
| ATOM | 5476 | N | ILE | B | 232 | −3.524 | 44.821 | 62.366 | 1.00 | 59.19 | B |
| ATOM | 5477 | CA | ILE | B | 232 | −4.498 | 44.222 | 63.263 | 1.00 | 59.19 | B |
| ATOM | 5478 | CB | ILE | B | 232 | −4.108 | 42.769 | 63.620 | 1.00 | 80.54 | B |
| ATOM | 5479 | CG2 | ILE | B | 232 | −4.906 | 42.277 | 64.816 | 1.00 | 50.59 | B |
| ATOM | 5480 | CG1 | ILE | B | 232 | −4.357 | 41.857 | 62.426 | 1.00 | 50.59 | B |
| ATOM | 5481 | CD1 | ILE | B | 232 | −3.976 | 40.411 | 62.691 | 1.00 | 50.59 | B |
| ATOM | 5482 | C | ILE | B | 232 | −4.640 | 45.020 | 64.555 | 1.00 | 59.19 | B |
| ATOM | 5483 | O | ILE | B | 232 | −5.722 | 45.074 | 65.142 | 1.00 | 59.19 | B |
| ATOM | 5484 | N | ASN | B | 233 | −3.558 | 45.644 | 64.999 | 1.00 | 80.45 | B |
| ATOM | 5485 | CA | ASN | B | 233 | −3.607 | 46.402 | 66.237 | 1.00 | 80.45 | B |
| ATOM | 5486 | CB | ASN | B | 233 | −2.224 | 46.451 | 66.886 | 1.00 | 79.11 | B |
| ATOM | 5487 | CG | ASN | B | 233 | −1.803 | 45.108 | 67.463 | 1.00 | 68.03 | B |
| ATOM | 5488 | OD1 | ASN | B | 233 | −2.639 | 44.322 | 67.916 | 1.00 | 68.03 | B |
| ATOM | 5489 | ND2 | ASN | B | 233 | −0.501 | 44.847 | 67.467 | 1.00 | 68.03 | B |
| ATOM | 5490 | C | ASN | B | 233 | −4.169 | 47.815 | 66.127 | 1.00 | 80.45 | B |
| ATOM | 5491 | O | ASN | B | 233 | −4.433 | 48.456 | 67.148 | 1.00 | 80.45 | B |
| ATOM | 5492 | N | LYS | B | 234 | −4.363 | 48.311 | 64.908 | 1.00 | 90.85 | B |
| ATOM | 5493 | CA | LYS | B | 234 | −4.914 | 49.653 | 64.746 | 1.00 | 90.85 | B |
| ATOM | 5494 | CB | LYS | B | 234 | −4.802 | 50.110 | 63.294 | 1.00 | 63.67 | B |
| ATOM | 5495 | CG | LYS | B | 234 | −3.373 | 50.460 | 62.920 | 1.00 | 63.67 | B |
| ATOM | 5496 | CD | LYS | B | 234 | −3.246 | 50.940 | 61.491 | 1.00 | 63.67 | B |
| ATOM | 5497 | CE | LYS | B | 234 | −1.801 | 51.315 | 61.192 | 1.00 | 63.67 | B |
| ATOM | 5498 | NZ | LYS | B | 234 | −1.579 | 51.662 | 59.755 | 1.00 | 63.67 | B |
| ATOM | 5499 | C | LYS | B | 234 | −6.356 | 49.720 | 65.243 | 1.00 | 90.85 | B |
| ATOM | 5500 | O | LYS | B | 234 | −6.714 | 50.660 | 65.945 | 1.00 | 90.85 | B |
| ATOM | 5501 | N | ASN | B | 235 | −7.186 | 48.743 | 64.882 | 1.00 | 92.11 | B |
| ATOM | 5502 | CA | ASN | B | 235 | −8.553 | 48.719 | 65.394 | 1.00 | 92.11 | B |
| ATOM | 5503 | CB | ASN | B | 235 | −9.447 | 47.748 | 64.601 | 1.00 | 149.06 | B |
| ATOM | 5504 | CG | ASN | B | 235 | −10.842 | 47.597 | 65.219 | 1.00 | 149.06 | B |
| ATOM | 5505 | OD1 | ASN | B | 235 | −10.961 | 47.479 | 66.438 | 1.00 | 149.06 | B |
| ATOM | 5506 | ND2 | ASN | B | 235 | −11.898 | 47.589 | 64.403 | 1.00 | 149.06 | B |
| ATOM | 5507 | C | ASN | B | 235 | −8.340 | 48.199 | 66.819 | 1.00 | 92.11 | B |
| ATOM | 5508 | O | ASN | B | 235 | −8.014 | 47.030 | 67.018 | 1.00 | 92.11 | B |
| ATOM | 5509 | N | ASP | B | 236 | −8.510 | 49.069 | 67.806 | 1.00 | 95.64 | B |
| ATOM | 5510 | CA | ASP | B | 236 | −8.287 | 48.699 | 69.197 | 1.00 | 95.64 | B |
| ATOM | 5511 | CB | ASP | B | 236 | −8.478 | 49.935 | 70.082 | 1.00 | 136.32 | B |
| ATOM | 5512 | CG | ASP | B | 236 | −7.709 | 49.845 | 71.386 | 1.00 | 136.32 | B |
| ATOM | 5513 | OD1 | ASP | B | 236 | −8.088 | 49.033 | 72.255 | 1.00 | 136.32 | B |
| ATOM | 5514 | OD2 | ASP | B | 236 | −6.717 | 50.586 | 71.538 | 1.00 | 136.32 | B |
| ATOM | 5515 | C | ASP | B | 236 | −9.124 | 47.531 | 69.739 | 1.00 | 95.64 | B |
| ATOM | 5516 | O | ASP | B | 236 | −8.888 | 47.069 | 70.857 | 1.00 | 95.64 | B |
| ATOM | 5517 | N | SER | B | 237 | −10.088 | 47.043 | 68.963 | 1.00 | 94.13 | B |
| ATOM | 5518 | CA | SER | B | 237 | −10.919 | 45.927 | 69.421 | 1.00 | 94.13 | B |
| ATOM | 5519 | CB | SER | B | 237 | −12.354 | 46.079 | 68.914 | 1.00 | 111.52 | B |
| ATOM | 5520 | OG | SER | B | 237 | −12.445 | 45.757 | 67.537 | 1.00 | 111.52 | B |
| ATOM | 5521 | C | SER | B | 237 | −10.366 | 44.578 | 68.961 | 1.00 | 94.13 | B |
| ATOM | 5522 | O | SER | B | 237 | −10.979 | 43.534 | 69.190 | 1.00 | 94.13 | B |
| ATOM | 5523 | N | THR | B | 238 | −9.208 | 44.609 | 68.306 | 1.00 | 98.32 | B |
| ATOM | 5524 | CA | THR | B | 238 | −8.560 | 43.394 | 67.816 | 1.00 | 98.32 | B |
| ATOM | 5525 | CB | THR | B | 238 | −8.397 | 43.416 | 66.282 | 1.00 | 95.39 | B |
| ATOM | 5526 | OG1 | THR | B | 238 | −9.608 | 43.871 | 65.676 | 1.00 | 95.39 | B |
| ATOM | 5527 | CG2 | THR | B | 238 | −8.081 | 42.017 | 65.761 | 1.00 | 95.39 | B |
| ATOM | 5528 | C | THR | B | 238 | −7.167 | 43.261 | 68.429 | 1.00 | 98.32 | B |
| ATOM | 5529 | O | THR | B | 238 | −6.635 | 42.160 | 68.542 | 1.00 | 98.32 | B |
| ATOM | 5530 | N | LYS | B | 239 | −6.592 | 44.398 | 68.812 | 1.00 | 94.58 | B |
| ATOM | 5531 | CA | LYS | B | 239 | −5.263 | 44.457 | 69.409 | 1.00 | 94.58 | B |
| ATOM | 5532 | CB | LYS | B | 239 | −5.219 | 45.567 | 70.463 | 1.00 | 102.41 | B |
| ATOM | 5533 | CG | LYS | B | 239 | −3.851 | 45.772 | 71.101 | 1.00 | 102.41 | B |
| ATOM | 5534 | CD | LYS | B | 239 | −3.816 | 47.040 | 71.934 | 1.00 | 102.41 | B |
| ATOM | 5535 | CE | LYS | B | 239 | −4.039 | 48.269 | 71.065 | 1.00 | 102.41 | B |
| ATOM | 5536 | NZ | LYS | B | 239 | −4.010 | 49.520 | 71.871 | 1.00 | 102.41 | B |
| ATOM | 5537 | C | LYS | B | 239 | −4.852 | 43.125 | 70.028 | 1.00 | 94.58 | B |
| ATOM | 5538 | O | LYS | B | 239 | −5.546 | 42.595 | 70.895 | 1.00 | 94.58 | B |
| ATOM | 5539 | N | THR | B | 240 | −3.722 | 42.589 | 69.568 | 1.00 | 59.35 | B |
| ATOM | 5540 | CA | THR | B | 240 | −3.213 | 41.309 | 70.053 | 1.00 | 59.35 | B |
| ATOM | 5541 | CB | THR | B | 240 | −3.427 | 40.190 | 69.024 | 1.00 | 77.87 | B |
| ATOM | 5542 | OG1 | THR | B | 240 | −4.803 | 40.153 | 68.638 | 1.00 | 77.87 | B |
| ATOM | 5543 | CG2 | THR | B | 240 | −3.052 | 38.851 | 69.617 | 1.00 | 77.87 | B |
| ATOM | 5544 | C | THR | B | 240 | −1.725 | 41.354 | 70.363 | 1.00 | 59.35 | B |
| ATOM | 5545 | O | THR | B | 240 | −0.978 | 42.145 | 69.796 | 1.00 | 59.35 | B |
| ATOM | 5546 | N | THR | B | 241 | −1.312 | 40.478 | 71.268 | 1.00 | 64.60 | B |
| ATOM | 5547 | CA | THR | B | 241 | 0.069 | 40.365 | 71.688 | 1.00 | 64.60 | B |
| ATOM | 5548 | CB | THR | B | 241 | 0.183 | 39.408 | 72.876 | 1.00 | 112.33 | B |
| ATOM | 5549 | OG1 | THR | B | 241 | −0.780 | 39.771 | 73.863 | 1.00 | 72.03 | B |
| ATOM | 5550 | CG2 | THR | B | 241 | 1.567 | 39.460 | 73.480 | 1.00 | 72.03 | B |
| ATOM | 5551 | C | THR | B | 241 | 0.911 | 39.786 | 70.557 | 1.00 | 64.60 | B |
| ATOM | 5552 | O | THR | B | 241 | 2.006 | 40.270 | 70.276 | 1.00 | 64.60 | B |
| ATOM | 5553 | N | SER | B | 242 | 0.392 | 38.748 | 69.907 | 1.00 | 56.39 | B |
| ATOM | 5554 | CA | SER | B | 242 | 1.129 | 38.076 | 68.849 | 1.00 | 56.39 | B |
| ATOM | 5555 | CB | SER | B | 242 | 1.953 | 36.940 | 69.450 | 1.00 | 96.71 | B |
| ATOM | 5556 | OG | SER | B | 242 | 1.093 | 35.991 | 70.054 | 1.00 | 69.23 | B |
| ATOM | 5557 | C | SER | B | 242 | 0.266 | 37.503 | 67.733 | 1.00 | 56.39 | B |
| ATOM | 5558 | O | SER | B | 242 | −0.964 | 37.453 | 67.822 | 1.00 | 56.39 | B |
| ATOM | 5559 | N | LEU | B | 243 | 0.950 | 37.055 | 66.685 | 1.00 | 53.64 | B |
| ATOM | 5560 | CA | LEU | B | 243 | 0.322 | 36.463 | 65.518 | 1.00 | 53.64 | B |
| ATOM | 5561 | CB | LEU | B | 243 | 0.342 | 37.470 | 64.381 | 1.00 | 61.90 | B |
| ATOM | 5562 | CG | LEU | B | 243 | −0.509 | 37.129 | 63.163 | 1.00 | 38.03 | B |
| ATOM | 5563 | CD1 | LEU | B | 243 | −1.949 | 36.968 | 63.606 | 1.00 | 38.03 | B |
| ATOM | 5564 | CD2 | LEU | B | 243 | −0.403 | 38.224 | 62.123 | 1.00 | 38.03 | B |
| ATOM | 5565 | C | LEU | B | 243 | 1.105 | 35.202 | 65.123 | 1.00 | 53.64 | B |
| ATOM | 5566 | O | LEU | B | 243 | 2.339 | 35.191 | 65.158 | 1.00 | 53.64 | B |
| ATOM | 5567 | N | SER | B | 244 | 0.394 | 34.140 | 64.759 | 1.00 | 50.57 | B |
| ATOM | 5568 | CA | SER | B | 244 | 1.053 | 32.901 | 64.374 | 1.00 | 50.57 | B |
| ATOM | 5569 | CB | SER | B | 244 | 0.879 | 31.836 | 65.458 | 1.00 | 44.31 | B |
| ATOM | 5570 | OG | SER | B | 244 | 1.513 | 32.196 | 66.668 | 1.00 | 46.46 | B |
| ATOM | 5571 | C | SER | B | 244 | 0.553 | 32.323 | 63.058 | 1.00 | 50.57 | B |
| ATOM | 5572 | O | SER | B | 244 | −0.635 | 32.397 | 62.739 | 1.00 | 50.57 | B |
| ATOM | 5573 | N | LEU | B | 245 | 1.477 | 31.755 | 62.290 | 1.00 | 44.59 | B |
| ATOM | 5574 | CA | LEU | B | 245 | 1.141 | 31.098 | 61.035 | 1.00 | 44.59 | B |
| ATOM | 5575 | CB | LEU | B | 245 | 2.028 | 31.604 | 59.910 | 1.00 | 42.30 | B |
| ATOM | 5576 | CG | LEU | B | 245 | 1.782 | 30.881 | 58.592 | 1.00 | 26.29 | B |
| ATOM | 5577 | CD1 | LEU | B | 245 | 0.308 | 31.012 | 58.177 | 1.00 | 26.29 | B |
| ATOM | 5578 | CD2 | LEU | B | 245 | 2.699 | 31.450 | 57.530 | 1.00 | 26.29 | B |
| ATOM | 5579 | C | LEU | B | 245 | 1.387 | 29.597 | 61.267 | 1.00 | 44.59 | B |
| ATOM | 5580 | O | LEU | B | 245 | 2.528 | 29.159 | 61.377 | 1.00 | 44.59 | B |
| ATOM | 5581 | N | ILE | B | 246 | 0.310 | 28.825 | 61.374 | 1.00 | 39.61 | B |
| ATOM | 5582 | CA | ILE | B | 246 | 0.406 | 27.393 | 61.614 | 1.00 | 39.61 | B |
| ATOM | 5583 | CB | ILE | B | 246 | −0.610 | 26.946 | 62.677 | 1.00 | 48.92 | B |
| ATOM | 5584 | CG2 | ILE | B | 246 | −0.449 | 25.470 | 62.979 | 1.00 | 31.96 | B |
| ATOM | 5585 | CG1 | ILE | B | 246 | −0.410 | 27.767 | 63.954 | 1.00 | 31.96 | B |
| ATOM | 5586 | CD1 | ILE | B | 246 | 0.991 | 27.695 | 64.519 | 1.00 | 31.96 | B |
| ATOM | 5587 | C | ILE | B | 246 | 0.124 | 26.641 | 60.333 | 1.00 | 39.61 | B |
| ATOM | 5588 | O | ILE | B | 246 | −0.965 | 26.743 | 59.780 | 1.00 | 39.61 | B |
| ATOM | 5589 | N | THR | B | 247 | 1.100 | 25.886 | 59.850 | 1.00 | 38.19 | B |
| ATOM | 5590 | CA | THR | B | 247 | 0.908 | 25.134 | 58.626 | 1.00 | 38.19 | B |
| ATOM | 5591 | CB | THR | B | 247 | 1.407 | 25.922 | 57.421 | 1.00 | 39.28 | B |
| ATOM | 5592 | OG1 | THR | B | 247 | 0.976 | 25.264 | 56.230 | 1.00 | 36.52 | B |
| ATOM | 5593 | CG2 | THR | B | 247 | 2.928 | 26.024 | 57.428 | 1.00 | 36.52 | B |
| ATOM | 5594 | C | THR | B | 247 | 1.663 | 23.821 | 58.733 | 1.00 | 38.19 | B |
| ATOM | 5595 | O | THR | B | 247 | 2.683 | 23.748 | 59.419 | 1.00 | 38.19 | B |
| ATOM | 5596 | N | GLY | B | 248 | 1.154 | 22.783 | 58.073 | 1.00 | 34.37 | B |
| ATOM | 5597 | CA | GLY | B | 248 | 1.794 | 21.477 | 58.136 | 1.00 | 34.37 | B |
| ATOM | 5598 | C | GLY | B | 248 | 0.877 | 20.381 | 57.631 | 1.00 | 34.37 | B |
| ATOM | 5599 | O | GLY | B | 248 | −0.180 | 20.671 | 57.063 | 1.00 | 34.37 | B |
| ATOM | 5600 | N | CYS | B | 249 | 1.264 | 19.125 | 57.842 | 1.00 | 36.28 | B |
| ATOM | 5601 | CA | CYS | B | 249 | 0.447 | 18.019 | 57.371 | 1.00 | 36.28 | B |
| ATOM | 5602 | CB | CYS | B | 249 | 0.871 | 17.612 | 55.962 | 1.00 | 37.70 | B |
| ATOM | 5603 | SG | CYS | B | 249 | 2.438 | 16.763 | 55.945 | 1.00 | 56.47 | B |
| ATOM | 5604 | C | CYS | B | 249 | 0.554 | 16.819 | 58.281 | 1.00 | 36.28 | B |
| ATOM | 5605 | O | CYS | B | 249 | 1.285 | 16.847 | 59.262 | 1.00 | 36.28 | B |
| ATOM | 5606 | N | TYR | B | 250 | −0.163 | 15.753 | 57.949 | 1.00 | 42.71 | B |
| ATOM | 5607 | CA | TYR | B | 250 | −0.142 | 14.560 | 58.775 | 1.00 | 42.71 | B |
| ATOM | 5608 | CB | TYR | B | 250 | −1.089 | 14.741 | 59.941 | 1.00 | 41.47 | B |
| ATOM | 5609 | CG | TYR | B | 250 | −2.520 | 14.924 | 59.492 | 1.00 | 31.82 | B |
| ATOM | 5610 | CD1 | TYR | B | 250 | −3.374 | 13.829 | 59.340 | 1.00 | 31.82 | B |
| ATOM | 5611 | CE1 | TYR | B | 250 | −4.681 | 13.994 | 58.892 | 1.00 | 31.82 | B |
| ATOM | 5612 | CD2 | TYR | B | 250 | −3.012 | 16.187 | 59.187 | 1.00 | 31.82 | B |
| ATOM | 5613 | CE2 | TYR | B | 250 | −4.310 | 16.367 | 58.738 | 1.00 | 31.82 | B |
| ATOM | 5614 | CZ | TYR | B | 250 | −5.145 | 15.271 | 58.588 | 1.00 | 31.82 | B |
| ATOM | 5615 | OH | TYR | B | 250 | −6.430 | 15.461 | 58.108 | 1.00 | 31.82 | B |
| ATOM | 5616 | C | TYR | B | 250 | −0.554 | 13.302 | 58.032 | 1.00 | 42.71 | B |
| ATOM | 5617 | O | TYR | B | 250 | −1.270 | 13.359 | 57.033 | 1.00 | 42.71 | B |
| ATOM | 5618 | N | GLU | B | 251 | −0.082 | 12.173 | 58.546 | 1.00 | 54.93 | B |
| ATOM | 5619 | CA | GLU | B | 251 | −0.397 | 10.858 | 58.033 | 1.00 | 54.93 | B |
| ATOM | 5620 | CB | GLU | B | 251 | 0.768 | 10.280 | 57.241 | 1.00 | 102.62 | B |
| ATOM | 5621 | CG | GLU | B | 251 | 0.447 | 10.141 | 55.776 | 1.00 | 56.24 | B |
| ATOM | 5622 | CD | GLU | B | 251 | 1.520 | 9.406 | 54.990 | 1.00 | 56.24 | B |
| ATOM | 5623 | OE1 | GLU | B | 251 | 1.854 | 8.258 | 55.367 | 1.00 | 56.24 | B |
| ATOM | 5624 | OE2 | GLU | B | 251 | 2.024 | 9.971 | 53.986 | 1.00 | 56.24 | B |
| ATOM | 5625 | C | GLU | B | 251 | −0.636 | 10.050 | 59.305 | 1.00 | 54.93 | B |
| ATOM | 5626 | O | GLU | B | 251 | 0.258 | 9.353 | 59.799 | 1.00 | 54.93 | B |
| ATOM | 5627 | N | THR | B | 252 | −1.842 | 10.190 | 59.852 | 1.00 | 46.01 | B |
| ATOM | 5628 | CA | THR | B | 252 | −2.248 | 9.505 | 61.076 | 1.00 | 46.01 | B |
| ATOM | 5629 | CB | THR | B | 252 | −3.164 | 10.415 | 61.928 | 1.00 | 53.02 | B |
| ATOM | 5630 | OG1 | THR | B | 252 | −2.526 | 11.680 | 62.139 | 1.00 | 53.02 | B |
| ATOM | 5631 | CG2 | THR | B | 252 | −3.459 | 9.782 | 63.260 | 1.00 | 53.02 | B |
| ATOM | 5632 | C | THR | B | 252 | −3.026 | 8.261 | 60.662 | 1.00 | 46.01 | B |
| ATOM | 5633 | O | THR | B | 252 | −4.212 | 8.339 | 60.360 | 1.00 | 46.01 | B |
| ATOM | 5634 | N | GLY | B | 253 | −2.357 | 7.115 | 60.646 | 1.00 | 61.07 | B |
| ATOM | 5635 | CA | GLY | B | 253 | −3.031 | 5.901 | 60.234 | 1.00 | 61.07 | B |
| ATOM | 5636 | C | GLY | B | 253 | −3.498 | 6.060 | 58.797 | 1.00 | 61.07 | B |
| ATOM | 5637 | O | GLY | B | 253 | −2.727 | 6.459 | 57.926 | 1.00 | 61.07 | B |
| ATOM | 5638 | N | LEU | B | 254 | −4.763 | 5.758 | 58.542 | 1.00 | 56.64 | B |
| ATOM | 5639 | CA | LEU | B | 254 | −5.313 | 5.890 | 57.201 | 1.00 | 56.64 | B |
| ATOM | 5640 | CB | LEU | B | 254 | −6.631 | 5.119 | 57.059 | 1.00 | 48.07 | B |
| ATOM | 5641 | CG | LEU | B | 254 | −6.599 | 3.599 | 57.091 | 1.00 | 48.07 | B |
| ATOM | 5642 | CD1 | LEU | B | 254 | −7.987 | 3.096 | 56.758 | 1.00 | 48.07 | B |
| ATOM | 5643 | CD2 | LEU | B | 254 | −5.576 | 3.071 | 56.109 | 1.00 | 48.07 | B |
| ATOM | 5644 | C | LEU | B | 254 | −5.588 | 7.337 | 56.851 | 1.00 | 56.64 | B |
| ATOM | 5645 | O | LEU | B | 254 | −5.583 | 7.697 | 55.676 | 1.00 | 56.64 | B |
| ATOM | 5646 | N | LEU | B | 255 | −5.853 | 8.158 | 57.864 | 1.00 | 43.85 | B |
| ATOM | 5647 | CA | LEU | B | 255 | −6.160 | 9.568 | 57.636 | 1.00 | 43.85 | B |
| ATOM | 5648 | CB | LEU | B | 255 | −6.757 | 10.187 | 58.898 | 1.00 | 54.86 | B |
| ATOM | 5649 | CG | LEU | B | 255 | −7.953 | 9.457 | 59.510 | 1.00 | 54.86 | B |
| ATOM | 5650 | CD1 | LEU | B | 255 | −8.469 | 10.251 | 60.705 | 1.00 | 54.86 | B |
| ATOM | 5651 | CD2 | LEU | B | 255 | −9.049 | 9.291 | 58.476 | 1.00 | 54.86 | B |
| ATOM | 5652 | C | LEU | B | 255 | −4.934 | 10.368 | 57.212 | 1.00 | 43.85 | B |
| ATOM | 5653 | O | LEU | B | 255 | −3.805 | 10.048 | 57.579 | 1.00 | 43.85 | B |
| ATOM | 5654 | N | PHE | B | 256 | −5.161 | 11.405 | 56.423 | 1.00 | 37.72 | B |
| ATOM | 5655 | CA | PHE | B | 256 | −4.069 | 12.244 | 55.969 | 1.00 | 37.72 | B |
| ATOM | 5656 | CB | PHE | B | 256 | −3.274 | 11.525 | 54.875 | 1.00 | 46.41 | B |
| ATOM | 5657 | CG | PHE | B | 256 | −4.040 | 11.305 | 53.602 | 1.00 | 43.42 | B |
| ATOM | 5658 | CD1 | PHE | B | 256 | −3.931 | 12.193 | 52.539 | 1.00 | 43.42 | B |
| ATOM | 5659 | CD2 | PHE | B | 256 | −4.869 | 10.196 | 53.456 | 1.00 | 43.42 | B |
| ATOM | 5660 | CE1 | PHE | B | 256 | −4.631 | 11.975 | 51.355 | 1.00 | 43.42 | B |
| ATOM | 5661 | CE2 | PHE | B | 256 | −5.578 | 9.976 | 52.262 | 1.00 | 43.42 | B |
| ATOM | 5662 | CZ | PHE | B | 256 | −5.456 | 10.862 | 51.220 | 1.00 | 43.42 | B |
| ATOM | 5663 | C | PHE | B | 256 | −4.615 | 13.570 | 55.457 | 1.00 | 37.72 | B |
| ATOM | 5664 | O | PHE | B | 256 | −5.692 | 13.613 | 54.871 | 1.00 | 37.72 | B |
| ATOM | 5665 | N | GLY | B | 257 | −3.881 | 14.651 | 55.700 | 1.00 | 42.86 | B |
| ATOM | 5666 | CA | GLY | B | 257 | −4.315 | 15.958 | 55.242 | 1.00 | 42.86 | B |
| ATOM | 5667 | C | GLY | B | 257 | −3.277 | 17.019 | 55.536 | 1.00 | 42.86 | B |
| ATOM | 5668 | O | GLY | B | 257 | −2.215 | 16.712 | 56.080 | 1.00 | 42.86 | B |
| ATOM | 5669 | N | SER | B | 258 | −3.579 | 18.265 | 55.177 | 1.00 | 42.61 | B |
| ATOM | 5670 | CA | SER | B | 258 | −2.661 | 19.366 | 55.414 | 1.00 | 42.61 | B |
| ATOM | 5671 | CB | SER | B | 258 | −1.914 | 19.720 | 54.136 | 1.00 | 39.72 | B |
| ATOM | 5672 | OG | SER | B | 258 | −2.804 | 20.269 | 53.187 | 1.00 | 49.84 | B |
| ATOM | 5673 | C | SER | B | 258 | −3.441 | 20.577 | 55.901 | 1.00 | 42.61 | B |
| ATOM | 5674 | O | SER | B | 258 | −4.656 | 20.644 | 55.750 | 1.00 | 42.61 | B |
| ATOM | 5675 | N | TYR | B | 259 | −2.746 | 21.541 | 56.483 | 1.00 | 35.88 | B |
| ATOM | 5676 | CA | TYR | B | 259 | −3.423 | 22.717 | 56.983 | 1.00 | 35.88 | B |
| ATOM | 5677 | CB | TYR | B | 259 | −3.878 | 22.473 | 58.426 | 1.00 | 47.90 | B |
| ATOM | 5678 | CG | TYR | B | 259 | −2.752 | 22.017 | 59.313 | 1.00 | 43.65 | B |
| ATOM | 5679 | CD1 | TYR | B | 259 | −1.840 | 22.935 | 59.848 | 1.00 | 43.65 | B |
| ATOM | 5680 | CE1 | TYR | B | 259 | −0.735 | 22.514 | 60.572 | 1.00 | 43.65 | B |
| ATOM | 5681 | CD2 | TYR | B | 259 | −2.529 | 20.664 | 59.537 | 1.00 | 43.65 | B |
| ATOM | 5682 | CE2 | TYR | B | 259 | −1.426 | 20.234 | 60.256 | 1.00 | 43.65 | B |
| ATOM | 5683 | CZ | TYR | B | 259 | −0.530 | 21.162 | 60.767 | 1.00 | 43.65 | B |
| ATOM | 5684 | OH | TYR | B | 259 | 0.591 | 20.726 | 61.436 | 1.00 | 43.65 | B |
| ATOM | 5685 | C | TYR | B | 259 | −2.547 | 23.957 | 56.922 | 1.00 | 35.88 | B |
| ATOM | 5686 | O | TYR | B | 259 | −1.322 | 23.883 | 56.782 | 1.00 | 35.88 | B |
| ATOM | 5687 | N | GLY | B | 260 | −3.201 | 25.102 | 57.035 | 1.00 | 40.11 | B |
| ATOM | 5688 | CA | GLY | B | 260 | −2.503 | 26.367 | 57.001 | 1.00 | 40.11 | B |
| ATOM | 5689 | C | GLY | B | 260 | −3.474 | 27.426 | 57.486 | 1.00 | 40.11 | B |
| ATOM | 5690 | O | GLY | B | 260 | −4.399 | 27.818 | 56.771 | 1.00 | 40.11 | B |
| ATOM | 5691 | N | TYR | B | 261 | −3.281 | 27.881 | 58.715 | 1.00 | 36.98 | B |
| ATOM | 5692 | CA | TYR | B | 261 | −4.156 | 28.891 | 59.261 | 1.00 | 36.98 | B |
| ATOM | 5693 | CB | TYR | B | 261 | −5.252 | 28.237 | 60.085 | 1.00 | 53.26 | B |
| ATOM | 5694 | CG | TYR | B | 261 | −4.758 | 27.438 | 61.261 | 1.00 | 37.05 | B |
| ATOM | 5695 | CD1 | TYR | B | 261 | −4.520 | 28.043 | 62.490 | 1.00 | 37.05 | B |
| ATOM | 5696 | CE1 | TYR | B | 261 | −4.106 | 27.300 | 63.593 | 1.00 | 37.05 | B |
| ATOM | 5697 | CD2 | TYR | B | 261 | −4.561 | 26.072 | 61.158 | 1.00 | 37.05 | B |
| ATOM | 5698 | CE2 | TYR | B | 261 | −4.147 | 25.318 | 62.245 | 1.00 | 37.05 | B |
| ATOM | 5699 | CZ | TYR | B | 261 | −3.923 | 25.932 | 63.463 | 1.00 | 37.05 | B |
| ATOM | 5700 | OH | TYR | B | 261 | −3.536 | 25.168 | 64.555 | 1.00 | 37.05 | B |
| ATOM | 5701 | C | TYR | B | 261 | −3.389 | 29.893 | 60.098 | 1.00 | 36.98 | B |
| ATOM | 5702 | O | TYR | B | 261 | −2.234 | 29.663 | 60.479 | 1.00 | 36.98 | B |
| ATOM | 5703 | N | VAL | B | 262 | −4.040 | 31.022 | 60.360 | 1.00 | 45.03 | B |
| ATOM | 5704 | CA | VAL | B | 262 | −3.449 | 32.093 | 61.142 | 1.00 | 45.03 | B |
| ATOM | 5705 | CB | VAL | B | 262 | −3.496 | 33.417 | 60.371 | 1.00 | 41.87 | B |
| ATOM | 5706 | CG1 | VAL | B | 262 | −3.125 | 34.558 | 61.290 | 1.00 | 33.36 | B |
| ATOM | 5707 | CG2 | VAL | B | 262 | −2.537 | 33.368 | 59.201 | 1.00 | 33.36 | B |
| ATOM | 5708 | C | VAL | B | 262 | −4.219 | 32.265 | 62.432 | 1.00 | 45.03 | B |
| ATOM | 5709 | O | VAL | B | 262 | −5.418 | 32.472 | 62.398 | 1.00 | 45.03 | B |
| ATOM | 5710 | N | GLU | B | 263 | −3.542 | 32.160 | 63.571 | 1.00 | 55.07 | B |
| ATOM | 5711 | CA | GLU | B | 263 | −4.209 | 32.350 | 64.852 | 1.00 | 55.07 | B |
| ATOM | 5712 | CB | GLU | B | 263 | −4.159 | 31.096 | 65.723 | 1.00 | 86.76 | B |
| ATOM | 5713 | CG | GLU | B | 263 | −2.792 | 30.538 | 65.954 | 1.00 | 73.36 | B |
| ATOM | 5714 | CD | GLU | B | 263 | −2.823 | 29.299 | 66.831 | 1.00 | 73.36 | B |
| ATOM | 5715 | OE1 | GLU | B | 263 | −3.737 | 28.454 | 66.656 | 1.00 | 73.36 | B |
| ATOM | 5716 | OE2 | GLU | B | 263 | −1.919 | 29.165 | 67.687 | 1.00 | 73.36 | B |
| ATOM | 5717 | C | GLU | B | 263 | −3.593 | 33.514 | 65.586 | 1.00 | 55.07 | B |
| ATOM | 5718 | O | GLU | B | 263 | −2.471 | 33.934 | 65.301 | 1.00 | 55.07 | B |
| ATOM | 5719 | N | THR | B | 264 | −4.358 | 34.036 | 66.531 | 1.00 | 59.77 | B |
| ATOM | 5720 | CA | THR | B | 264 | −3.979 | 35.191 | 67.324 | 1.00 | 59.77 | B |
| ATOM | 5721 | CB | THR | B | 264 | −5.047 | 36.285 | 67.123 | 1.00 | 79.64 | B |
| ATOM | 5722 | OG1 | THR | B | 264 | −4.564 | 37.238 | 66.175 | 1.00 | 58.57 | B |
| ATOM | 5723 | CG2 | THR | B | 264 | −5.404 | 36.967 | 68.405 | 1.00 | 58.57 | B |
| ATOM | 5724 | C | THR | B | 264 | −3.888 | 34.779 | 68.778 | 1.00 | 59.77 | B |
| ATOM | 5725 | O | THR | B | 264 | −4.321 | 33.685 | 69.134 | 1.00 | 59.77 | B |
| ATOM | 5726 | N | ALA | B | 265 | −3.309 | 35.628 | 69.620 | 1.00 | 71.63 | B |
| ATOM | 5727 | CA | ALA | B | 265 | −3.217 | 35.301 | 71.037 | 1.00 | 71.63 | B |
| ATOM | 5728 | CB | ALA | B | 265 | −2.477 | 36.395 | 71.784 | 1.00 | 89.03 | B |
| ATOM | 5729 | C | ALA | B | 265 | −4.636 | 35.146 | 71.586 | 1.00 | 71.63 | B |
| ATOM | 5730 | O | ALA | B | 265 | −4.851 | 34.487 | 72.600 | 1.00 | 71.63 | B |
| ATOM | 5731 | N | ASN | B | 266 | −5.599 | 35.749 | 70.893 | 1.00 | 92.80 | B |
| ATOM | 5732 | CA | ASN | B | 266 | −7.003 | 35.691 | 71.281 | 1.00 | 92.80 | B |
| ATOM | 5733 | CB | ASN | B | 266 | −7.742 | 36.928 | 70.786 | 1.00 | 144.08 | B |
| ATOM | 5734 | CG | ASN | B | 266 | −7.089 | 38.201 | 71.242 | 1.00 | 85.93 | B |
| ATOM | 5735 | OD1 | ASN | B | 266 | −6.825 | 38.378 | 72.431 | 1.00 | 85.93 | B |
| ATOM | 5736 | ND2 | ASN | B | 266 | −6.821 | 39.102 | 70.302 | 1.00 | 85.93 | B |
| ATOM | 5737 | C | ASN | B | 266 | −7.704 | 34.453 | 70.747 | 1.00 | 92.80 | B |
| ATOM | 5738 | O | ASN | B | 266 | −8.804 | 34.139 | 71.181 | 1.00 | 92.80 | B |
| ATOM | 5739 | N | GLY | B | 267 | −7.077 | 33.763 | 69.798 | 1.00 | 53.93 | B |
| ATOM | 5740 | CA | GLY | B | 267 | −7.665 | 32.555 | 69.244 | 1.00 | 53.93 | B |
| ATOM | 5741 | C | GLY | B | 267 | −7.614 | 32.473 | 67.737 | 1.00 | 53.93 | B |
| ATOM | 5742 | O | GLY | B | 267 | −7.225 | 33.434 | 67.087 | 1.00 | 53.93 | B |
| ATOM | 5743 | N | PRO | B | 268 | −8.033 | 31.340 | 67.150 | 1.00 | 55.79 | B |
| ATOM | 5744 | CD | PRO | B | 268 | −8.797 | 30.267 | 67.807 | 1.00 | 42.67 | B |
| ATOM | 5745 | CA | PRO | B | 268 | −8.023 | 31.146 | 65.696 | 1.00 | 55.79 | B |
| ATOM | 5746 | CB | PRO | B | 268 | −8.826 | 29.862 | 65.508 | 1.00 | 58.56 | B |
| ATOM | 5747 | CG | PRO | B | 268 | −9.719 | 29.832 | 66.701 | 1.00 | 42.67 | B |
| ATOM | 5748 | C | PRO | B | 268 | −8.612 | 32.316 | 64.945 | 1.00 | 55.79 | B |
| ATOM | 5749 | O | PRO | B | 268 | −9.639 | 32.852 | 65.335 | 1.00 | 55.79 | B |
| ATOM | 5750 | N | LEU | B | 269 | −7.949 | 32.708 | 63.866 | 1.00 | 61.57 | B |
| ATOM | 5751 | CA | LEU | B | 269 | −8.389 | 33.830 | 63.063 | 1.00 | 61.57 | B |
| ATOM | 5752 | CB | LEU | B | 269 | −7.237 | 34.825 | 62.919 | 1.00 | 91.30 | B |
| ATOM | 5753 | CG | LEU | B | 269 | −7.572 | 36.238 | 62.447 | 1.00 | 56.64 | B |
| ATOM | 5754 | CD1 | LEU | B | 269 | −8.635 | 36.828 | 63.351 | 1.00 | 56.64 | B |
| ATOM | 5755 | CD2 | LEU | B | 269 | −6.322 | 37.107 | 62.465 | 1.00 | 56.64 | B |
| ATOM | 5756 | C | LEU | B | 269 | −8.904 | 33.398 | 61.686 | 1.00 | 61.57 | B |
| ATOM | 5757 | O | LEU | B | 269 | −10.110 | 33.412 | 61.438 | 1.00 | 61.57 | B |
| ATOM | 5758 | N | ALA | B | 270 | −8.000 | 33.005 | 60.795 | 1.00 | 51.91 | B |
| ATOM | 5759 | CA | ALA | B | 270 | −8.390 | 32.600 | 59.444 | 1.00 | 51.91 | B |
| ATOM | 5760 | CB | ALA | B | 270 | −8.185 | 33.770 | 58.466 | 1.00 | 25.82 | B |
| ATOM | 5761 | C | ALA | B | 270 | −7.656 | 31.363 | 58.929 | 1.00 | 51.91 | B |
| ATOM | 5762 | O | ALA | B | 270 | −6.467 | 31.154 | 59.192 | 1.00 | 51.91 | B |
| ATOM | 5763 | N | ARG | B | 271 | −8.380 | 30.570 | 58.154 | 1.00 | 52.73 | B |
| ATOM | 5764 | CA | ARG | B | 271 | −7.875 | 29.329 | 57.584 | 1.00 | 52.73 | B |
| ATOM | 5765 | CB | ARG | B | 271 | −8.876 | 28.224 | 57.923 | 1.00 | 51.88 | B |
| ATOM | 5766 | CG | ARG | B | 271 | −8.678 | 26.939 | 57.215 | 1.00 | 45.00 | B |
| ATOM | 5767 | CD | ARG | B | 271 | −9.784 | 25.983 | 57.575 | 1.00 | 45.00 | B |
| ATOM | 5768 | NE | ARG | B | 271 | −9.694 | 25.519 | 58.955 | 1.00 | 45.00 | B |
| ATOM | 5769 | CZ | ARG | B | 271 | −10.507 | 25.893 | 59.934 | 1.00 | 45.00 | B |
| ATOM | 5770 | NH1 | ARG | B | 271 | −11.487 | 26.750 | 59.696 | 1.00 | 45.00 | B |
| ATOM | 5771 | NH2 | ARG | B | 271 | −10.346 | 25.394 | 61.148 | 1.00 | 45.00 | B |
| ATOM | 5772 | C | ARG | B | 271 | −7.709 | 29.490 | 56.073 | 1.00 | 52.73 | B |
| ATOM | 5773 | O | ARG | B | 271 | −8.568 | 30.060 | 55.412 | 1.00 | 52.73 | B |
| ATOM | 5774 | N | TYR | B | 272 | −6.602 | 29.003 | 55.527 | 1.00 | 44.31 | B |
| ATOM | 5775 | CA | TYR | B | 272 | −6.355 | 29.134 | 54.090 | 1.00 | 44.31 | B |
| ATOM | 5776 | CB | TYR | B | 272 | −4.858 | 29.287 | 53.812 | 1.00 | 44.63 | B |
| ATOM | 5777 | CG | TYR | B | 272 | −4.541 | 29.474 | 52.342 | 1.00 | 42.10 | B |
| ATOM | 5778 | CD1 | TYR | B | 272 | −4.643 | 30.727 | 51.739 | 1.00 | 42.10 | B |
| ATOM | 5779 | CE1 | TYR | B | 272 | −4.379 | 30.897 | 50.381 | 1.00 | 42.10 | B |
| ATOM | 5780 | CD2 | TYR | B | 272 | −4.163 | 28.394 | 51.547 | 1.00 | 42.10 | B |
| ATOM | 5781 | CE2 | TYR | B | 272 | −3.895 | 28.557 | 50.190 | 1.00 | 42.10 | B |
| ATOM | 5782 | CZ | TYR | B | 272 | −4.007 | 29.809 | 49.614 | 1.00 | 42.10 | B |
| ATOM | 5783 | OH | TYR | B | 272 | −3.767 | 29.987 | 48.268 | 1.00 | 42.10 | B |
| ATOM | 5784 | C | TYR | B | 272 | −6.864 | 27.935 | 53.320 | 1.00 | 44.31 | B |
| ATOM | 5785 | O | TYR | B | 272 | −6.451 | 26.814 | 53.580 | 1.00 | 44.31 | B |
| ATOM | 5786 | N | HIS | B | 273 | −7.750 | 28.161 | 52.361 | 1.00 | 59.19 | B |
| ATOM | 5787 | CA | HIS | B | 273 | −8.282 | 27.057 | 51.576 | 1.00 | 59.19 | B |
| ATOM | 5788 | CB | HIS | B | 273 | −9.793 | 27.193 | 51.406 | 1.00 | 141.23 | B |
| ATOM | 5789 | CG | HIS | B | 273 | −10.558 | 26.850 | 52.643 | 1.00 | 121.96 | B |
| ATOM | 5790 | CD2 | HIS | B | 273 | −11.395 | 27.586 | 53.411 | 1.00 | 121.96 | B |
| ATOM | 5791 | ND1 | HIS | B | 273 | −10.471 | 25.615 | 53.248 | 1.00 | 121.96 | B |
| ATOM | 5792 | CE1 | HIS | B | 273 | −11.221 | 25.605 | 54.336 | 1.00 | 121.96 | B |
| ATOM | 5793 | NE2 | HIS | B | 273 | −11.792 | 26.790 | 54.458 | 1.00 | 121.96 | B |
| ATOM | 5794 | C | HIS | B | 273 | −7.613 | 26.920 | 50.227 | 1.00 | 59.19 | B |
| ATOM | 5795 | O | HIS | B | 273 | −7.636 | 27.838 | 49.410 | 1.00 | 59.19 | B |
| ATOM | 5796 | N | THR | B | 274 | −6.992 | 25.762 | 50.022 | 1.00 | 44.66 | B |
| ATOM | 5797 | CA | THR | B | 274 | −6.303 | 25.430 | 48.782 | 1.00 | 44.66 | B |
| ATOM | 5798 | CB | THR | B | 274 | −5.754 | 23.997 | 48.877 | 1.00 | 74.54 | B |
| ATOM | 5799 | OG1 | THR | B | 274 | −4.377 | 24.056 | 49.259 | 1.00 | 67.07 | B |
| ATOM | 5800 | CG2 | THR | B | 274 | −5.915 | 23.247 | 47.562 | 1.00 | 67.07 | B |
| ATOM | 5801 | C | THR | B | 274 | −7.221 | 25.572 | 47.562 | 1.00 | 44.66 | B |
| ATOM | 5802 | O | THR | B | 274 | −6.796 | 26.029 | 46.504 | 1.00 | 44.66 | B |
| ATOM | 5803 | N | GLY | B | 275 | −8.481 | 25.184 | 47.717 | 1.00 | 61.42 | B |
| ATOM | 5804 | CA | GLY | B | 275 | −9.424 | 25.288 | 46.620 | 1.00 | 61.42 | B |
| ATOM | 5805 | C | GLY | B | 275 | −9.718 | 26.717 | 46.191 | 1.00 | 61.42 | B |
| ATOM | 5806 | O | GLY | B | 275 | −9.398 | 27.110 | 45.072 | 1.00 | 61.42 | B |
| ATOM | 5807 | N | ASP | B | 276 | −10.322 | 27.494 | 47.087 | 1.00 | 57.53 | B |
| ATOM | 5808 | CA | ASP | B | 276 | −10.685 | 28.880 | 46.812 | 1.00 | 57.53 | B |
| ATOM | 5809 | CB | ASP | B | 276 | −11.575 | 29.409 | 47.937 | 1.00 | 119.51 | B |
| ATOM | 5810 | CG | ASP | B | 276 | −12.714 | 28.470 | 48.263 | 1.00 | 98.51 | B |
| ATOM | 5811 | OD1 | ASP | B | 276 | −13.623 | 28.319 | 47.421 | 1.00 | 98.51 | B |
| ATOM | 5812 | OD2 | ASP | B | 276 | −12.693 | 27.873 | 49.361 | 1.00 | 98.51 | B |
| ATOM | 5813 | C | ASP | B | 276 | −9.491 | 29.819 | 46.647 | 1.00 | 57.53 | B |
| ATOM | 5814 | O | ASP | B | 276 | −9.642 | 30.914 | 46.107 | 1.00 | 57.53 | B |
| ATOM | 5815 | N | LYS | B | 277 | −8.315 | 29.400 | 47.115 | 1.00 | 66.28 | B |
| ATOM | 5816 | CA | LYS | B | 277 | −7.101 | 30.221 | 47.041 | 1.00 | 66.28 | B |
| ATOM | 5817 | CB | LYS | B | 277 | −6.720 | 30.467 | 45.579 | 1.00 | 82.20 | B |
| ATOM | 5818 | CG | LYS | B | 277 | −6.276 | 29.208 | 44.851 | 1.00 | 67.41 | B |
| ATOM | 5819 | CD | LYS | B | 277 | −6.220 | 29.425 | 43.351 | 1.00 | 67.41 | B |
| ATOM | 5820 | CE | LYS | B | 277 | −5.728 | 28.187 | 42.616 | 1.00 | 67.41 | B |
| ATOM | 5821 | NZ | LYS | B | 277 | −4.255 | 27.992 | 42.773 | 1.00 | 67.41 | B |
| ATOM | 5822 | C | LYS | B | 277 | −7.293 | 31.552 | 47.772 | 1.00 | 66.28 | B |
| ATOM | 5823 | O | LYS | B | 277 | −6.998 | 32.618 | 47.237 | 1.00 | 66.28 | B |
| ATOM | 5824 | N | ARG | B | 278 | −7.794 | 31.467 | 49.002 | 1.00 | 47.39 | B |
| ATOM | 5825 | CA | ARG | B | 278 | −8.057 | 32.639 | 49.832 | 1.00 | 47.39 | B |
| ATOM | 5826 | CB | ARG | B | 278 | −9.308 | 33.351 | 49.334 | 1.00 | 104.52 | B |
| ATOM | 5827 | CG | ARG | B | 278 | −10.572 | 32.575 | 49.615 | 1.00 | 89.15 | B |
| ATOM | 5828 | CD | ARG | B | 278 | −11.760 | 33.163 | 48.896 | 1.00 | 89.15 | B |
| ATOM | 5829 | NE | ARG | B | 278 | −12.998 | 32.476 | 49.258 | 1.00 | 89.15 | B |
| ATOM | 5830 | CZ | ARG | B | 278 | −14.138 | 32.579 | 48.581 | 1.00 | 89.15 | B |
| ATOM | 5831 | NH1 | ARG | B | 278 | −14.199 | 33.344 | 47.497 | 1.00 | 89.15 | B |
| ATOM | 5832 | NH2 | ARG | B | 278 | −15.217 | 31.917 | 48.985 | 1.00 | 89.15 | B |
| ATOM | 5833 | C | ARG | B | 278 | −8.280 | 32.192 | 51.283 | 1.00 | 47.39 | B |
| ATOM | 5834 | O | ARG | B | 278 | −8.436 | 31.002 | 51.560 | 1.00 | 47.39 | B |
| ATOM | 5835 | N | PHE | B | 279 | −8.294 | 33.139 | 52.211 | 1.00 | 53.81 | B |
| ATOM | 5836 | CA | PHE | B | 279 | −8.512 | 32.784 | 53.606 | 1.00 | 53.81 | B |
| ATOM | 5837 | CB | PHE | B | 279 | −7.813 | 33.768 | 54.544 | 1.00 | 53.49 | B |
| ATOM | 5838 | CG | PHE | B | 279 | −6.350 | 33.518 | 54.703 | 1.00 | 37.26 | B |
| ATOM | 5839 | CD1 | PHE | B | 279 | −5.424 | 34.164 | 53.891 | 1.00 | 37.26 | B |
| ATOM | 5840 | CD2 | PHE | B | 279 | −5.889 | 32.641 | 55.674 | 1.00 | 37.26 | B |
| ATOM | 5841 | CE1 | PHE | B | 279 | −4.056 | 33.940 | 54.050 | 1.00 | 37.26 | B |
| ATOM | 5842 | CE2 | PHE | B | 279 | −4.524 | 32.411 | 55.837 | 1.00 | 37.26 | B |
| ATOM | 5843 | CZ | PHE | B | 279 | −3.608 | 33.062 | 55.025 | 1.00 | 37.26 | B |
| ATOM | 5844 | C | PHE | B | 279 | −9.996 | 32.778 | 53.927 | 1.00 | 53.81 | B |
| ATOM | 5845 | O | PHE | B | 279 | −10.780 | 33.468 | 53.279 | 1.00 | 53.81 | B |
| ATOM | 5846 | N | THR | B | 280 | −10.375 | 32.002 | 54.937 | 1.00 | 63.30 | B |
| ATOM | 5847 | CA | THR | B | 280 | −11.764 | 31.921 | 55.358 | 1.00 | 63.30 | B |
| ATOM | 5848 | CB | THR | B | 280 | −12.409 | 30.619 | 54.907 | 1.00 | 105.89 | B |
| ATOM | 5849 | OG1 | THR | B | 280 | −12.183 | 30.439 | 53.507 | 1.00 | 86.41 | B |
| ATOM | 5850 | CG2 | THR | B | 280 | −13.900 | 30.658 | 55.172 | 1.00 | 86.41 | B |
| ATOM | 5851 | C | THR | B | 280 | −11.842 | 31.976 | 56.873 | 1.00 | 63.30 | B |
| ATOM | 5852 | O | THR | B | 280 | −11.036 | 31.355 | 57.561 | 1.00 | 63.30 | B |
| ATOM | 5853 | N | LYS | B | 281 | −12.815 | 32.723 | 57.383 | 1.00 | 50.98 | B |
| ATOM | 5854 | CA | LYS | B | 281 | −13.023 | 32.864 | 58.818 | 1.00 | 50.98 | B |
| ATOM | 5855 | CB | LYS | B | 281 | −14.334 | 33.612 | 59.066 | 1.00 | 110.08 | B |
| ATOM | 5856 | CG | LYS | B | 281 | −15.431 | 33.249 | 58.067 | 1.00 | 86.02 | B |
| ATOM | 5857 | CD | LYS | B | 281 | −16.752 | 32.890 | 58.745 | 1.00 | 86.02 | B |
| ATOM | 5858 | CE | LYS | B | 281 | −17.376 | 34.075 | 59.466 | 1.00 | 86.02 | B |
| ATOM | 5859 | NZ | LYS | B | 281 | −18.691 | 33.715 | 60.074 | 1.00 | 86.02 | B |
| ATOM | 5860 | C | LYS | B | 281 | −13.060 | 31.505 | 59.530 | 1.00 | 50.98 | B |
| ATOM | 5861 | O | LYS | B | 281 | −13.598 | 30.530 | 59.005 | 1.00 | 50.98 | B |
| ATOM | 5862 | N | MET | B | 282 | −12.468 | 31.441 | 60.720 | 1.00 | 53.53 | B |
| ATOM | 5863 | CA | MET | B | 282 | −12.460 | 30.214 | 61.511 | 1.00 | 53.53 | B |
| ATOM | 5864 | CB | MET | B | 282 | −11.076 | 29.936 | 62.092 | 1.00 | 66.15 | B |
| ATOM | 5865 | CG | MET | B | 282 | −10.032 | 29.505 | 61.084 | 1.00 | 66.15 | B |
| ATOM | 5866 | SD | MET | B | 282 | −8.582 | 28.753 | 61.888 | 1.00 | 66.15 | B |
| ATOM | 5867 | CE | MET | B | 282 | −7.605 | 30.165 | 62.126 | 1.00 | 66.15 | B |
| ATOM | 5868 | C | MET | B | 282 | −13.432 | 30.380 | 62.663 | 1.00 | 53.53 | B |
| ATOM | 5869 | O | MET | B | 282 | −13.717 | 29.428 | 63.376 | 1.00 | 53.53 | B |
| ATOM | 5870 | N | THR | B | 283 | −13.922 | 31.605 | 62.836 | 1.00 | 68.75 | B |
| ATOM | 5871 | CA | THR | B | 283 | −14.856 | 31.955 | 63.897 | 1.00 | 68.75 | B |
| ATOM | 5872 | CB | THR | B | 283 | −14.143 | 32.744 | 65.004 | 1.00 | 83.27 | B |
| ATOM | 5873 | OG1 | THR | B | 283 | −13.158 | 31.911 | 65.617 | 1.00 | 83.27 | B |
| ATOM | 5874 | CG2 | THR | B | 283 | −15.123 | 33.206 | 66.059 | 1.00 | 83.27 | B |
| ATOM | 5875 | C | THR | B | 283 | −15.956 | 32.836 | 63.318 | 1.00 | 68.75 | B |
| ATOM | 5876 | O | THR | B | 283 | −15.773 | 33.465 | 62.274 | 1.00 | 68.75 | B |
| ATOM | 5877 | N | HIS | B | 284 | −17.101 | 32.877 | 63.995 | 1.00 | 86.80 | B |
| ATOM | 5878 | CA | HIS | B | 284 | −18.226 | 33.702 | 63.560 | 1.00 | 86.80 | B |
| ATOM | 5879 | CB | HIS | B | 284 | −19.375 | 33.585 | 64.569 | 1.00 | 143.17 | B |
| ATOM | 5880 | CG | HIS | B | 284 | −18.984 | 33.937 | 65.974 | 1.00 | 143.17 | B |
| ATOM | 5881 | CD2 | HIS | B | 284 | −19.381 | 34.948 | 66.783 | 1.00 | 143.17 | B |
| ATOM | 5882 | ND1 | HIS | B | 284 | −18.055 | 33.215 | 66.694 | 1.00 | 143.17 | B |
| ATOM | 5883 | CE1 | HIS | B | 284 | −17.896 | 33.766 | 67.884 | 1.00 | 143.17 | B |
| ATOM | 5884 | NE2 | HIS | B | 284 | −18.689 | 34.820 | 67.964 | 1.00 | 143.17 | B |
| ATOM | 5885 | C | HIS | B | 284 | −17.752 | 35.152 | 63.483 | 1.00 | 86.80 | B |
| ATOM | 5886 | O | HIS | B | 284 | −18.139 | 35.903 | 62.592 | 1.00 | 86.80 | B |
| ATOM | 5887 | N | LYS | B | 285 | −16.889 | 35.513 | 64.428 | 1.00 | 70.99 | B |
| ATOM | 5888 | CA | LYS | B | 285 | −16.326 | 36.851 | 64.552 | 1.00 | 70.99 | B |
| ATOM | 5889 | CB | LYS | B | 285 | −15.223 | 36.842 | 65.613 | 1.00 | 109.80 | B |
| ATOM | 5890 | CG | LYS | B | 285 | −15.357 | 37.921 | 66.670 | 1.00 | 109.80 | B |
| ATOM | 5891 | CD | LYS | B | 285 | −16.604 | 37.717 | 67.518 | 1.00 | 109.80 | B |
| ATOM | 5892 | CE | LYS | B | 285 | −16.733 | 38.792 | 68.592 | 1.00 | 109.80 | B |
| ATOM | 5893 | NZ | LYS | B | 285 | −17.940 | 38.595 | 69.441 | 1.00 | 109.80 | B |
| ATOM | 5894 | C | LYS | B | 285 | −15.766 | 37.426 | 63.257 | 1.00 | 70.99 | B |
| ATOM | 5895 | O | LYS | B | 285 | −15.802 | 38.638 | 63.056 | 1.00 | 70.99 | B |
| ATOM | 5896 | N | GLY | B | 286 | −15.248 | 36.564 | 62.386 | 1.00 | 79.92 | B |
| ATOM | 5897 | CA | GLY | B | 286 | −14.664 | 37.032 | 61.137 | 1.00 | 79.92 | B |
| ATOM | 5898 | C | GLY | B | 286 | −13.210 | 37.421 | 61.351 | 1.00 | 79.92 | B |
| ATOM | 5899 | O | GLY | B | 286 | −12.679 | 37.247 | 62.450 | 1.00 | 79.92 | B |
| ATOM | 5900 | N | PHE | B | 287 | −12.555 | 37.938 | 60.314 | 1.00 | 70.66 | B |
| ATOM | 5901 | CA | PHE | B | 287 | −11.153 | 38.353 | 60.428 | 1.00 | 70.66 | B |
| ATOM | 5902 | CB | PHE | B | 287 | −10.216 | 37.220 | 59.998 | 1.00 | 41.30 | B |
| ATOM | 5903 | CG | PHE | B | 287 | −10.252 | 36.914 | 58.524 | 1.00 | 41.30 | B |
| ATOM | 5904 | CD1 | PHE | B | 287 | −9.468 | 37.642 | 57.628 | 1.00 | 41.30 | B |
| ATOM | 5905 | CD2 | PHE | B | 287 | −11.081 | 35.898 | 58.023 | 1.00 | 41.30 | B |
| ATOM | 5906 | CE1 | PHE | B | 287 | −9.511 | 37.360 | 56.241 | 1.00 | 41.30 | B |
| ATOM | 5907 | CE2 | PHE | B | 287 | −11.128 | 35.613 | 56.647 | 1.00 | 41.30 | B |
| ATOM | 5908 | CZ | PHE | B | 287 | −10.346 | 36.342 | 55.755 | 1.00 | 41.30 | B |
| ATOM | 5909 | C | PHE | B | 287 | −10.866 | 39.598 | 59.600 | 1.00 | 70.66 | B |
| ATOM | 5910 | O | PHE | B | 287 | −11.373 | 39.751 | 58.491 | 1.00 | 70.66 | B |
| ATOM | 5911 | N | PRO | B | 288 | −10.036 | 40.507 | 60.130 | 1.00 | 56.08 | B |
| ATOM | 5912 | CD | PRO | B | 288 | −9.306 | 40.386 | 61.407 | 1.00 | 66.61 | B |
| ATOM | 5913 | CA | PRO | B | 288 | −9.681 | 41.753 | 59.437 | 1.00 | 56.08 | B |
| ATOM | 5914 | CB | PRO | B | 288 | −8.860 | 42.506 | 60.478 | 1.00 | 66.61 | B |
| ATOM | 5915 | CG | PRO | B | 288 | −8.189 | 41.396 | 61.234 | 1.00 | 66.61 | B |
| ATOM | 5916 | C | PRO | B | 288 | −8.912 | 41.515 | 58.156 | 1.00 | 56.08 | B |
| ATOM | 5917 | O | PRO | B | 288 | −7.885 | 40.859 | 58.161 | 1.00 | 56.08 | B |
| ATOM | 5918 | N | LYS | B | 289 | −9.417 | 42.042 | 57.051 | 1.00 | 65.90 | B |
| ATOM | 5919 | CA | LYS | B | 289 | −8.750 | 41.869 | 55.770 | 1.00 | 65.90 | B |
| ATOM | 5920 | CB | LYS | B | 289 | −9.755 | 41.990 | 54.620 | 1.00 | 100.30 | B |
| ATOM | 5921 | CG | LYS | B | 289 | −10.603 | 40.733 | 54.455 | 1.00 | 100.30 | B |
| ATOM | 5922 | CD | LYS | B | 289 | −11.604 | 40.860 | 53.326 | 1.00 | 100.30 | B |
| ATOM | 5923 | CE | LYS | B | 289 | −12.727 | 41.831 | 53.683 | 1.00 | 100.30 | B |
| ATOM | 5924 | NZ | LYS | B | 289 | −13.752 | 41.951 | 52.596 | 1.00 | 100.30 | B |
| ATOM | 5925 | C | LYS | B | 289 | −7.615 | 42.865 | 55.617 | 1.00 | 65.90 | B |
| ATOM | 5926 | O | LYS | B | 289 | −7.736 | 43.872 | 54.927 | 1.00 | 65.90 | B |
| ATOM | 5927 | N | VAL | B | 290 | −6.502 | 42.550 | 56.273 | 1.00 | 54.57 | B |
| ATOM | 5928 | CA | VAL | B | 290 | −5.300 | 43.373 | 56.262 | 1.00 | 54.57 | B |
| ATOM | 5929 | CB | VAL | B | 290 | −5.171 | 44.171 | 57.574 | 1.00 | 52.01 | B |
| ATOM | 5930 | CG1 | VAL | B | 290 | −6.461 | 44.909 | 57.850 | 1.00 | 52.01 | B |
| ATOM | 5931 | CG2 | VAL | B | 290 | −4.849 | 43.232 | 58.730 | 1.00 | 52.01 | B |
| ATOM | 5932 | C | VAL | B | 290 | −4.064 | 42.490 | 56.124 | 1.00 | 54.57 | B |
| ATOM | 5933 | O | VAL | B | 290 | −4.138 | 41.266 | 56.269 | 1.00 | 54.57 | B |
| ATOM | 5934 | N | GLY | B | 291 | −2.928 | 43.119 | 55.848 | 1.00 | 43.80 | B |
| ATOM | 5935 | CA | GLY | B | 291 | −1.682 | 42.384 | 55.711 | 1.00 | 43.80 | B |
| ATOM | 5936 | C | GLY | B | 291 | −1.712 | 41.162 | 54.809 | 1.00 | 43.80 | B |
| ATOM | 5937 | O | GLY | B | 291 | −2.198 | 41.217 | 53.684 | 1.00 | 43.80 | B |
| ATOM | 5938 | N | MET | B | 292 | −1.177 | 40.057 | 55.308 | 1.00 | 61.56 | B |
| ATOM | 5939 | CA | MET | B | 292 | −1.111 | 38.812 | 54.558 | 1.00 | 61.56 | B |
| ATOM | 5940 | CB | MET | B | 292 | −0.201 | 37.829 | 55.277 | 1.00 | 60.79 | B |
| ATOM | 5941 | CG | MET | B | 292 | −0.812 | 37.361 | 56.587 | 1.00 | 60.79 | B |
| ATOM | 5942 | SD | MET | B | 292 | −0.067 | 35.869 | 57.248 | 1.00 | 60.79 | B |
| ATOM | 5943 | CE | MET | B | 292 | 0.627 | 36.472 | 58.729 | 1.00 | 60.79 | B |
| ATOM | 5944 | C | MET | B | 292 | −2.479 | 38.158 | 54.415 | 1.00 | 61.56 | B |
| ATOM | 5945 | O | MET | B | 292 | −2.591 | 37.085 | 53.818 | 1.00 | 61.56 | B |
| ATOM | 5946 | N | LEU | B | 293 | −3.515 | 38.787 | 54.961 | 1.00 | 59.68 | B |
| ATOM | 5947 | CA | LEU | B | 293 | −4.846 | 38.206 | 54.899 | 1.00 | 59.68 | B |
| ATOM | 5948 | CB | LEU | B | 293 | −5.559 | 38.425 | 56.231 | 1.00 | 49.18 | B |
| ATOM | 5949 | CG | LEU | B | 293 | −4.922 | 37.725 | 57.430 | 1.00 | 49.18 | B |
| ATOM | 5950 | CD1 | LEU | B | 293 | −5.628 | 38.142 | 58.707 | 1.00 | 49.18 | B |
| ATOM | 5951 | CD2 | LEU | B | 293 | −5.006 | 36.218 | 57.235 | 1.00 | 49.18 | B |
| ATOM | 5952 | C | LEU | B | 293 | −5.758 | 38.649 | 53.756 | 1.00 | 59.68 | B |
| ATOM | 5953 | O | LEU | B | 293 | −6.859 | 38.126 | 53.626 | 1.00 | 59.68 | B |
| ATOM | 5954 | N | THR | B | 294 | −5.329 | 39.598 | 52.932 | 1.00 | 67.38 | B |
| ATOM | 5955 | CA | THR | B | 294 | −6.172 | 40.042 | 51.825 | 1.00 | 67.38 | B |
| ATOM | 5956 | CB | THR | B | 294 | −6.626 | 41.504 | 51.960 | 1.00 | 103.47 | B |
| ATOM | 5957 | OG1 | THR | B | 294 | −6.944 | 41.788 | 53.322 | 1.00 | 103.47 | B |
| ATOM | 5958 | CG2 | THR | B | 294 | −7.874 | 41.743 | 51.103 | 1.00 | 103.47 | B |
| ATOM | 5959 | C | THR | B | 294 | −5.482 | 39.954 | 50.483 | 1.00 | 67.38 | B |
| ATOM | 5960 | O | THR | B | 294 | −4.264 | 40.089 | 50.384 | 1.00 | 67.38 | B |
| ATOM | 5961 | N | VAL | B | 295 | −6.279 | 39.750 | 49.441 | 1.00 | 58.87 | B |
| ATOM | 5962 | CA | VAL | B | 295 | −5.747 | 39.668 | 48.095 | 1.00 | 58.87 | B |
| ATOM | 5963 | CB | VAL | B | 295 | −6.835 | 39.223 | 47.117 | 1.00 | 71.22 | B |
| ATOM | 5964 | CG1 | VAL | B | 295 | −6.239 | 39.046 | 45.726 | 1.00 | 71.22 | B |
| ATOM | 5965 | CG2 | VAL | B | 295 | −7.463 | 37.924 | 47.614 | 1.00 | 71.22 | B |
| ATOM | 5966 | C | VAL | B | 295 | −5.248 | 41.053 | 47.713 | 1.00 | 58.87 | B |
| ATOM | 5967 | O | VAL | B | 295 | −4.423 | 41.207 | 46.815 | 1.00 | 58.87 | B |
| ATOM | 5968 | N | LYS | B | 296 | −5.750 | 42.055 | 48.427 | 1.00 | 55.74 | B |
| ATOM | 5969 | CA | LYS | B | 296 | −5.385 | 43.450 | 48.189 | 1.00 | 55.74 | B |
| ATOM | 5970 | CB | LYS | B | 296 | −6.135 | 44.371 | 49.161 | 1.00 | 92.95 | B |
| ATOM | 5971 | CG | LYS | B | 296 | −7.649 | 44.311 | 49.068 | 1.00 | 92.95 | B |
| ATOM | 5972 | CD | LYS | B | 296 | −8.170 | 45.006 | 47.823 | 1.00 | 92.95 | B |
| ATOM | 5973 | CE | LYS | B | 296 | −7.982 | 46.523 | 47.909 | 1.00 | 92.95 | B |
| ATOM | 5974 | NZ | LYS | B | 296 | −8.466 | 47.251 | 46.689 | 1.00 | 92.95 | B |
| ATOM | 5975 | C | LYS | B | 296 | −3.889 | 43.666 | 48.365 | 1.00 | 55.74 | B |
| ATOM | 5976 | O | LYS | B | 296 | −3.301 | 44.509 | 47.694 | 1.00 | 55.74 | B |
| ATOM | 5977 | N | ASN | B | 297 | −3.281 | 42.892 | 49.264 | 1.00 | 57.39 | B |
| ATOM | 5978 | CA | ASN | B | 297 | −1.856 | 43.021 | 49.562 | 1.00 | 57.39 | B |
| ATOM | 5979 | CB | ASN | B | 297 | −1.463 | 42.097 | 50.716 | 1.00 | 69.42 | B |
| ATOM | 5980 | CG | ASN | B | 297 | −0.065 | 42.388 | 51.242 | 1.00 | 69.42 | B |
| ATOM | 5981 | OD1 | ASN | B | 297 | 0.926 | 42.269 | 50.522 | 1.00 | 69.42 | B |
| ATOM | 5982 | ND2 | ASN | B | 297 | 0.016 | 42.777 | 52.506 | 1.00 | 69.42 | B |
| ATOM | 5983 | C | ASN | B | 297 | −0.941 | 42.762 | 48.376 | 1.00 | 57.39 | B |
| ATOM | 5984 | O | ASN | B | 297 | −1.119 | 41.814 | 47.614 | 1.00 | 57.39 | B |
| ATOM | 5985 | N | THR | B | 298 | 0.055 | 43.620 | 48.238 | 1.00 | 58.64 | B |
| ATOM | 5986 | CA | THR | B | 298 | 1.005 | 43.514 | 47.150 | 1.00 | 58.64 | B |
| ATOM | 5987 | CB | THR | B | 298 | 1.983 | 44.713 | 47.197 | 1.00 | 62.16 | B |
| ATOM | 5988 | OG1 | THR | B | 298 | 3.172 | 44.397 | 46.465 | 1.00 | 62.16 | B |
| ATOM | 5989 | CG2 | THR | B | 298 | 2.339 | 45.052 | 48.632 | 1.00 | 62.16 | B |
| ATOM | 5990 | C | THR | B | 298 | 1.783 | 42.191 | 47.130 | 1.00 | 58.64 | B |
| ATOM | 5991 | O | THR | B | 298 | 2.230 | 41.744 | 46.078 | 1.00 | 58.64 | B |
| ATOM | 5992 | N | LEU | B | 299 | 1.930 | 41.553 | 48.284 | 1.00 | 46.95 | B |
| ATOM | 5993 | CA | LEU | B | 299 | 2.673 | 40.292 | 48.364 | 1.00 | 46.95 | B |
| ATOM | 5994 | CB | LEU | B | 299 | 3.545 | 40.279 | 49.614 | 1.00 | 46.04 | B |
| ATOM | 5995 | CG | LEU | B | 299 | 4.640 | 41.338 | 49.626 | 1.00 | 46.04 | B |
| ATOM | 5996 | CD1 | LEU | B | 299 | 5.308 | 41.353 | 50.974 | 1.00 | 46.04 | B |
| ATOM | 5997 | CD2 | LEU | B | 299 | 5.622 | 41.043 | 48.519 | 1.00 | 46.04 | B |
| ATOM | 5998 | C | LEU | B | 299 | 1.798 | 39.042 | 48.365 | 1.00 | 46.95 | B |
| ATOM | 5999 | O | LEU | B | 299 | 2.295 | 37.935 | 48.575 | 1.00 | 46.95 | B |
| ATOM | 6000 | N | TRP | B | 300 | 0.501 | 39.226 | 48.132 | 1.00 | 46.77 | B |
| ATOM | 6001 | CA | TRP | B | 300 | −0.444 | 38.121 | 48.099 | 1.00 | 46.77 | B |
| ATOM | 6002 | CB | TRP | B | 300 | −1.794 | 38.591 | 47.574 | 1.00 | 46.03 | B |
| ATOM | 6003 | CG | TRP | B | 300 | −2.804 | 37.514 | 47.553 | 1.00 | 46.03 | B |
| ATOM | 6004 | CD2 | TRP | B | 300 | −3.278 | 36.763 | 48.677 | 1.00 | 46.03 | B |
| ATOM | 6005 | CE2 | TRP | B | 300 | −4.222 | 35.836 | 48.190 | 1.00 | 46.03 | B |
| ATOM | 6006 | CE3 | TRP | B | 300 | −2.996 | 36.785 | 50.047 | 1.00 | 46.03 | B |
| ATOM | 6007 | CD1 | TRP | B | 300 | −3.458 | 37.029 | 46.465 | 1.00 | 46.03 | B |
| ATOM | 6008 | NE1 | TRP | B | 300 | −4.313 | 36.019 | 46.837 | 1.00 | 46.03 | B |
| ATOM | 6009 | CZ2 | TRP | B | 300 | −4.890 | 34.937 | 49.026 | 1.00 | 46.03 | B |
| ATOM | 6010 | CZ3 | TRP | B | 300 | −3.658 | 35.893 | 50.880 | 1.00 | 46.03 | B |
| ATOM | 6011 | CH2 | TRP | B | 300 | −4.598 | 34.978 | 50.364 | 1.00 | 46.03 | B |
| ATOM | 6012 | C | TRP | B | 300 | 0.047 | 36.977 | 47.231 | 1.00 | 46.77 | B |
| ATOM | 6013 | O | TRP | B | 300 | −0.092 | 35.808 | 47.612 | 1.00 | 46.77 | B |
| ATOM | 6014 | N | LYS | B | 301 | 0.618 | 37.308 | 46.071 | 1.00 | 36.79 | B |
| ATOM | 6015 | CA | LYS | B | 301 | 1.118 | 36.280 | 45.159 | 1.00 | 36.79 | B |
| ATOM | 6016 | CB | LYS | B | 301 | 1.733 | 36.911 | 43.904 | 1.00 | 96.13 | B |
| ATOM | 6017 | CG | LYS | B | 301 | 0.744 | 37.753 | 43.097 | 1.00 | 96.13 | B |
| ATOM | 6018 | CD | LYS | B | 301 | −0.592 | 37.023 | 42.865 | 1.00 | 96.13 | B |
| ATOM | 6019 | CE | LYS | B | 301 | −1.699 | 37.991 | 42.452 | 1.00 | 96.13 | B |
| ATOM | 6020 | NZ | LYS | B | 301 | −3.054 | 37.397 | 42.598 | 1.00 | 96.13 | B |
| ATOM | 6021 | C | LYS | B | 301 | 2.134 | 35.378 | 45.844 | 1.00 | 36.79 | B |
| ATOM | 6022 | O | LYS | B | 301 | 2.171 | 34.174 | 45.584 | 1.00 | 36.79 | B |
| ATOM | 6023 | N | ASP | B | 302 | 2.956 | 35.951 | 46.725 | 1.00 | 53.59 | B |
| ATOM | 6024 | CA | ASP | B | 302 | 3.948 | 35.162 | 47.442 | 1.00 | 53.59 | B |
| ATOM | 6025 | CB | ASP | B | 302 | 4.997 | 36.064 | 48.071 | 1.00 | 67.89 | B |
| ATOM | 6026 | CG | ASP | B | 302 | 5.927 | 36.665 | 47.041 | 1.00 | 67.89 | B |
| ATOM | 6027 | OD1 | ASP | B | 302 | 6.445 | 35.902 | 46.207 | 1.00 | 67.89 | B |
| ATOM | 6028 | OD2 | ASP | B | 302 | 6.149 | 37.892 | 47.064 | 1.00 | 67.89 | B |
| ATOM | 6029 | C | ASP | B | 302 | 3.275 | 34.304 | 48.502 | 1.00 | 53.59 | B |
| ATOM | 6030 | O | ASP | B | 302 | 3.680 | 33.168 | 48.745 | 1.00 | 53.59 | B |
| ATOM | 6031 | N | VAL | B | 303 | 2.238 | 34.841 | 49.126 | 1.00 | 39.74 | B |
| ATOM | 6032 | CA | VAL | B | 303 | 1.507 | 34.084 | 50.129 | 1.00 | 39.74 | B |
| ATOM | 6033 | CB | VAL | B | 303 | 0.363 | 34.910 | 50.730 | 1.00 | 38.02 | B |
| ATOM | 6034 | CG1 | VAL | B | 303 | −0.488 | 34.037 | 51.655 | 1.00 | 27.33 | B |
| ATOM | 6035 | CG2 | VAL | B | 303 | 0.943 | 36.101 | 51.495 | 1.00 | 27.33 | B |
| ATOM | 6036 | C | VAL | B | 303 | 0.926 | 32.832 | 49.474 | 1.00 | 39.74 | B |
| ATOM | 6037 | O | VAL | B | 303 | 1.125 | 31.715 | 49.961 | 1.00 | 39.74 | B |
| ATOM | 6038 | N | LYS | B | 304 | 0.211 | 33.022 | 48.368 | 1.00 | 43.65 | B |
| ATOM | 6039 | CA | LYS | B | 304 | −0.378 | 31.902 | 47.635 | 1.00 | 43.65 | B |
| ATOM | 6040 | CB | LYS | B | 304 | −0.958 | 32.373 | 46.306 | 1.00 | 86.72 | B |
| ATOM | 6041 | CG | LYS | B | 304 | −2.344 | 32.970 | 46.364 | 1.00 | 68.64 | B |
| ATOM | 6042 | CD | LYS | B | 304 | −2.753 | 33.440 | 44.972 | 1.00 | 68.64 | B |
| ATOM | 6043 | CE | LYS | B | 304 | −4.187 | 33.930 | 44.942 | 1.00 | 68.64 | B |
| ATOM | 6044 | NZ | LYS | B | 304 | −4.569 | 34.505 | 43.617 | 1.00 | 68.64 | B |
| ATOM | 6045 | C | LYS | B | 304 | 0.677 | 30.839 | 47.343 | 1.00 | 43.65 | B |
| ATOM | 6046 | O | LYS | B | 304 | 0.460 | 29.640 | 47.541 | 1.00 | 43.65 | B |
| ATOM | 6047 | N | ALA | B | 305 | 1.831 | 31.288 | 46.873 | 1.00 | 43.49 | B |
| ATOM | 6048 | CA | ALA | B | 305 | 2.920 | 30.389 | 46.522 | 1.00 | 43.49 | B |
| ATOM | 6049 | CB | ALA | B | 305 | 3.948 | 31.141 | 45.724 | 1.00 | 18.56 | B |
| ATOM | 6050 | C | ALA | B | 305 | 3.588 | 29.687 | 47.688 | 1.00 | 43.49 | B |
| ATOM | 6051 | O | ALA | B | 305 | 3.751 | 28.471 | 47.670 | 1.00 | 43.49 | B |
| ATOM | 6052 | N | TYR | B | 306 | 3.987 | 30.444 | 48.701 | 1.00 | 47.05 | B |
| ATOM | 6053 | CA | TYR | B | 306 | 4.647 | 29.847 | 49.859 | 1.00 | 47.05 | B |
| ATOM | 6054 | CB | TYR | B | 306 | 5.340 | 30.921 | 50.691 | 1.00 | 51.37 | B |
| ATOM | 6055 | CG | TYR | B | 306 | 6.491 | 31.575 | 49.982 | 1.00 | 46.06 | B |
| ATOM | 6056 | CD1 | TYR | B | 306 | 6.839 | 32.899 | 50.248 | 1.00 | 46.06 | B |
| ATOM | 6057 | CE1 | TYR | B | 306 | 7.889 | 33.516 | 49.581 | 1.00 | 46.06 | B |
| ATOM | 6058 | CD2 | TYR | B | 306 | 7.227 | 30.882 | 49.035 | 1.00 | 46.06 | B |
| ATOM | 6059 | CE2 | TYR | B | 306 | 8.278 | 31.485 | 48.362 | 1.00 | 46.06 | B |
| ATOM | 6060 | CZ | TYR | B | 306 | 8.607 | 32.800 | 48.636 | 1.00 | 46.06 | B |
| ATOM | 6061 | OH | TYR | B | 306 | 9.656 | 33.388 | 47.968 | 1.00 | 46.06 | B |
| ATOM | 6062 | C | TYR | B | 306 | 3.712 | 29.049 | 50.754 | 1.00 | 47.05 | B |
| ATOM | 6063 | O | TYR | B | 306 | 3.994 | 27.906 | 51.068 | 1.00 | 47.05 | B |
| ATOM | 6064 | N | LEU | B | 307 | 2.605 | 29.649 | 51.171 | 1.00 | 35.43 | B |
| ATOM | 6065 | CA | LEU | B | 307 | 1.659 | 28.968 | 52.040 | 1.00 | 35.43 | B |
| ATOM | 6066 | CB | LEU | B | 307 | 0.797 | 29.991 | 52.766 | 1.00 | 65.60 | B |
| ATOM | 6067 | CG | LEU | B | 307 | −0.373 | 29.417 | 53.563 | 1.00 | 33.73 | B |
| ATOM | 6068 | CD1 | LEU | B | 307 | 0.122 | 28.471 | 54.650 | 1.00 | 33.73 | B |
| ATOM | 6069 | CD2 | LEU | B | 307 | −1.150 | 30.563 | 54.177 | 1.00 | 33.73 | B |
| ATOM | 6070 | C | LEU | B | 307 | 0.762 | 28.014 | 51.263 | 1.00 | 35.43 | B |
| ATOM | 6071 | O | LEU | B | 307 | 0.634 | 26.847 | 51.620 | 1.00 | 35.43 | B |
| ATOM | 6072 | N | GLY | B | 308 | 0.133 | 28.517 | 50.205 | 1.00 | 38.82 | B |
| ATOM | 6073 | CA | GLY | B | 308 | −0.743 | 27.687 | 49.413 | 1.00 | 38.82 | B |
| ATOM | 6074 | C | GLY | B | 308 | −0.016 | 26.495 | 48.839 | 1.00 | 38.82 | B |
| ATOM | 6075 | O | GLY | B | 308 | −0.500 | 25.372 | 48.935 | 1.00 | 38.82 | B |
| ATOM | 6076 | N | GLY | B | 309 | 1.148 | 26.725 | 48.244 | 1.00 | 37.32 | B |
| ATOM | 6077 | CA | GLY | B | 309 | 1.896 | 25.623 | 47.657 | 1.00 | 37.32 | B |
| ATOM | 6078 | C | GLY | B | 309 | 2.259 | 24.514 | 48.634 | 1.00 | 37.32 | B |
| ATOM | 6079 | O | GLY | B | 309 | 2.057 | 23.323 | 48.361 | 1.00 | 37.32 | B |
| ATOM | 6080 | N | PHE | B | 310 | 2.798 | 24.915 | 49.783 | 1.00 | 43.11 | B |
| ATOM | 6081 | CA | PHE | B | 310 | 3.212 | 23.986 | 50.816 | 1.00 | 43.11 | B |
| ATOM | 6082 | CB | PHE | B | 310 | 3.763 | 24.772 | 51.998 | 1.00 | 40.30 | B |
| ATOM | 6083 | CG | PHE | B | 310 | 4.448 | 23.926 | 53.032 | 1.00 | 36.13 | B |
| ATOM | 6084 | CD1 | PHE | B | 310 | 5.573 | 23.179 | 52.702 | 1.00 | 36.13 | B |
| ATOM | 6085 | CD2 | PHE | B | 310 | 3.977 | 23.886 | 54.350 | 1.00 | 36.13 | B |
| ATOM | 6086 | CE1 | PHE | B | 310 | 6.226 | 22.402 | 53.664 | 1.00 | 36.13 | B |
| ATOM | 6087 | CE2 | PHE | B | 310 | 4.627 | 23.110 | 55.323 | 1.00 | 36.13 | B |
| ATOM | 6088 | CZ | PHE | B | 310 | 5.751 | 22.369 | 54.974 | 1.00 | 36.13 | B |
| ATOM | 6089 | C | PHE | B | 310 | 2.019 | 23.150 | 51.253 | 1.00 | 43.11 | B |
| ATOM | 6090 | O | PHE | B | 310 | 2.112 | 21.925 | 51.405 | 1.00 | 43.11 | B |
| ATOM | 6091 | N | GLU | B | 311 | 0.896 | 23.827 | 51.455 | 1.00 | 44.80 | B |
| ATOM | 6092 | CA | GLU | B | 311 | −0.320 | 23.171 | 51.893 | 1.00 | 44.80 | B |
| ATOM | 6093 | CB | GLU | B | 311 | −1.353 | 24.206 | 52.332 | 1.00 | 80.04 | B |
| ATOM | 6094 | CG | GLU | B | 311 | −2.394 | 23.646 | 53.276 | 1.00 | 64.27 | B |
| ATOM | 6095 | CD | GLU | B | 311 | −3.517 | 24.620 | 53.565 | 1.00 | 64.27 | B |
| ATOM | 6096 | OE1 | GLU | B | 311 | −3.252 | 25.833 | 53.682 | 1.00 | 64.27 | B |
| ATOM | 6097 | OE2 | GLU | B | 311 | −4.671 | 24.174 | 53.692 | 1.00 | 64.27 | B |
| ATOM | 6098 | C | GLU | B | 311 | −0.909 | 22.314 | 50.790 | 1.00 | 44.80 | B |
| ATOM | 6099 | O | GLU | B | 311 | −1.479 | 21.269 | 51.055 | 1.00 | 44.80 | B |
| ATOM | 6100 | N | TYR | B | 312 | −0.770 | 22.753 | 49.548 | 1.00 | 39.84 | B |
| ATOM | 6101 | CA | TYR | B | 312 | −1.324 | 21.999 | 48.437 | 1.00 | 39.84 | B |
| ATOM | 6102 | CB | TYR | B | 312 | −1.284 | 22.811 | 47.151 | 1.00 | 52.75 | B |
| ATOM | 6103 | CG | TYR | B | 312 | −1.927 | 22.084 | 45.998 | 1.00 | 47.45 | B |
| ATOM | 6104 | CD1 | TYR | B | 312 | −3.319 | 21.936 | 45.928 | 1.00 | 47.45 | B |
| ATOM | 6105 | CE1 | TYR | B | 312 | −3.925 | 21.232 | 44.876 | 1.00 | 47.45 | B |
| ATOM | 6106 | CD2 | TYR | B | 312 | −1.153 | 21.509 | 44.987 | 1.00 | 47.45 | B |
| ATOM | 6107 | CE2 | TYR | B | 312 | −1.748 | 20.798 | 43.932 | 1.00 | 47.45 | B |
| ATOM | 6108 | CZ | TYR | B | 312 | −3.135 | 20.663 | 43.882 | 1.00 | 47.45 | B |
| ATOM | 6109 | OH | TYR | B | 312 | −3.736 | 19.966 | 42.850 | 1.00 | 47.45 | B |
| ATOM | 6110 | C | TYR | B | 312 | −0.621 | 20.683 | 48.184 | 1.00 | 39.84 | B |
| ATOM | 6111 | O | TYR | B | 312 | −1.266 | 19.710 | 47.823 | 1.00 | 39.84 | B |
| ATOM | 6112 | N | MET | B | 313 | 0.696 | 20.642 | 48.361 | 1.00 | 42.19 | B |
| ATOM | 6113 | CA | MET | B | 313 | 1.422 | 19.409 | 48.101 | 1.00 | 42.19 | B |
| ATOM | 6114 | CB | MET | B | 313 | 1.525 | 19.182 | 46.594 | 1.00 | 64.59 | B |
| ATOM | 6115 | CG | MET | B | 313 | 2.204 | 17.894 | 46.220 | 1.00 | 64.44 | B |
| ATOM | 6116 | SD | MET | B | 313 | 2.388 | 17.757 | 44.452 | 1.00 | 64.44 | B |
| ATOM | 6117 | CE | MET | B | 313 | 0.696 | 17.888 | 43.913 | 1.00 | 64.44 | B |
| ATOM | 6118 | C | MET | B | 313 | 2.814 | 19.381 | 48.696 | 1.00 | 42.19 | B |
| ATOM | 6119 | O | MET | B | 313 | 3.300 | 18.326 | 49.108 | 1.00 | 42.19 | B |
| ATOM | 6120 | N | GLY | B | 314 | 3.461 | 20.541 | 48.726 | 1.00 | 42.13 | B |
| ATOM | 6121 | CA | GLY | B | 314 | 4.809 | 20.622 | 49.253 | 1.00 | 42.13 | B |
| ATOM | 6122 | C | GLY | B | 314 | 5.028 | 19.852 | 50.536 | 1.00 | 42.13 | B |
| ATOM | 6123 | O | GLY | B | 314 | 5.912 | 18.990 | 50.627 | 1.00 | 42.13 | B |
| ATOM | 6124 | N | CYS | B | 315 | 4.216 | 20.167 | 51.536 | 1.00 | 46.13 | B |
| ATOM | 6125 | CA | CYS | B | 315 | 4.342 | 19.516 | 52.820 | 1.00 | 46.13 | B |
| ATOM | 6126 | CB | CYS | B | 315 | 3.303 | 20.076 | 53.777 | 1.00 | 56.37 | B |
| ATOM | 6127 | SG | CYS | B | 315 | 3.661 | 19.641 | 55.475 | 1.00 | 61.74 | B |
| ATOM | 6128 | C | CYS | B | 315 | 4.226 | 17.986 | 52.753 | 1.00 | 46.13 | B |
| ATOM | 6129 | O | CYS | B | 315 | 5.106 | 17.270 | 53.245 | 1.00 | 46.13 | B |
| ATOM | 6130 | N | SER | B | 316 | 3.147 | 17.482 | 52.154 | 1.00 | 43.91 | B |
| ATOM | 6131 | CA | SER | B | 316 | 2.952 | 16.035 | 52.046 | 1.00 | 43.91 | B |
| ATOM | 6132 | CB | SER | B | 316 | 1.707 | 15.722 | 51.230 | 1.00 | 46.87 | B |
| ATOM | 6133 | OG | SER | B | 316 | 1.893 | 16.081 | 49.876 | 1.00 | 52.37 | B |
| ATOM | 6134 | C | SER | B | 316 | 4.145 | 15.316 | 51.413 | 1.00 | 43.91 | B |
| ATOM | 6135 | O | SER | B | 316 | 4.381 | 14.144 | 51.696 | 1.00 | 43.91 | B |
| ATOM | 6136 | N | LEU | B | 317 | 4.880 | 16.001 | 50.537 | 1.00 | 40.03 | B |
| ATOM | 6137 | CA | LEU | B | 317 | 6.056 | 15.408 | 49.920 | 1.00 | 40.03 | B |
| ATOM | 6138 | CB | LEU | B | 317 | 6.402 | 16.142 | 48.630 | 1.00 | 44.23 | B |
| ATOM | 6139 | CG | LEU | B | 317 | 5.427 | 15.943 | 47.469 | 1.00 | 36.74 | B |
| ATOM | 6140 | CD1 | LEU | B | 317 | 5.796 | 16.853 | 46.303 | 1.00 | 36.74 | B |
| ATOM | 6141 | CD2 | LEU | B | 317 | 5.453 | 14.487 | 47.040 | 1.00 | 36.74 | B |
| ATOM | 6142 | C | LEU | B | 317 | 7.240 | 15.495 | 50.895 | 1.00 | 40.03 | B |
| ATOM | 6143 | O | LEU | B | 317 | 7.986 | 14.538 | 51.084 | 1.00 | 40.03 | B |
| ATOM | 6144 | N | ALA | B | 318 | 7.393 | 16.645 | 51.535 | 1.00 | 37.43 | B |
| ATOM | 6145 | CA | ALA | B | 318 | 8.490 | 16.851 | 52.461 | 1.00 | 37.43 | B |
| ATOM | 6146 | CB | ALA | B | 318 | 8.484 | 18.293 | 52.949 | 1.00 | 37.40 | B |
| ATOM | 6147 | C | ALA | B | 318 | 8.439 | 15.899 | 53.647 | 1.00 | 37.43 | B |
| ATOM | 6148 | O | ALA | B | 318 | 9.451 | 15.288 | 54.013 | 1.00 | 37.43 | B |
| ATOM | 6149 | N | ILE | B | 319 | 7.263 | 15.771 | 54.253 | 1.00 | 37.68 | B |
| ATOM | 6150 | CA | ILE | B | 319 | 7.127 | 14.903 | 55.417 | 1.00 | 37.68 | B |
| ATOM | 6151 | CB | ILE | B | 319 | 5.650 | 14.720 | 55.822 | 1.00 | 49.36 | B |
| ATOM | 6152 | CG2 | ILE | B | 319 | 4.945 | 13.851 | 54.828 | 1.00 | 23.92 | B |
| ATOM | 6153 | CG1 | ILE | B | 319 | 5.573 | 14.082 | 57.217 | 1.00 | 23.92 | B |
| ATOM | 6154 | CD1 | ILE | B | 319 | 4.164 | 14.019 | 57.810 | 1.00 | 23.92 | B |
| ATOM | 6155 | C | ILE | B | 319 | 7.765 | 13.527 | 55.216 | 1.00 | 37.68 | B |
| ATOM | 6156 | O | ILE | B | 319 | 8.270 | 12.935 | 56.160 | 1.00 | 37.68 | B |
| ATOM | 6157 | N | LEU | B | 320 | 7.749 | 13.025 | 53.986 | 1.00 | 41.23 | B |
| ATOM | 6158 | CA | LEU | B | 320 | 8.340 | 11.719 | 53.698 | 1.00 | 41.23 | B |
| ATOM | 6159 | CB | LEU | B | 320 | 7.889 | 11.235 | 52.318 | 1.00 | 66.95 | B |
| ATOM | 6160 | CG | LEU | B | 320 | 6.381 | 10.970 | 52.170 | 1.00 | 44.79 | B |
| ATOM | 6161 | CD1 | LEU | B | 320 | 6.073 | 10.480 | 50.761 | 1.00 | 44.79 | B |
| ATOM | 6162 | CD2 | LEU | B | 320 | 5.934 | 9.934 | 53.192 | 1.00 | 44.79 | B |
| ATOM | 6163 | C | LEU | B | 320 | 9.878 | 11.732 | 53.793 | 1.00 | 41.23 | B |
| ATOM | 6164 | O | LEU | B | 320 | 10.490 | 10.728 | 54.151 | 1.00 | 41.23 | B |
| ATOM | 6165 | N | ASP | B | 321 | 10.501 | 12.870 | 53.481 | 1.00 | 53.93 | B |
| ATOM | 6166 | CA | ASP | B | 321 | 11.943 | 12.973 | 53.570 | 1.00 | 53.93 | B |
| ATOM | 6167 | CB | ASP | B | 321 | 12.436 | 14.235 | 52.872 | 1.00 | 90.40 | B |
| ATOM | 6168 | CG | ASP | B | 321 | 12.308 | 14.151 | 51.363 | 1.00 | 69.40 | B |
| ATOM | 6169 | OD1 | ASP | B | 321 | 12.647 | 13.088 | 50.812 | 1.00 | 69.40 | B |
| ATOM | 6170 | OD2 | ASP | B | 321 | 11.886 | 15.151 | 50.727 | 1.00 | 69.40 | B |
| ATOM | 6171 | C | ASP | B | 321 | 12.335 | 12.996 | 55.044 | 1.00 | 53.93 | B |
| ATOM | 6172 | O | ASP | B | 321 | 13.345 | 12.396 | 55.434 | 1.00 | 53.93 | B |
| ATOM | 6173 | N | TYR | B | 322 | 11.540 | 13.681 | 55.866 | 1.00 | 56.60 | B |
| ATOM | 6174 | CA | TYR | B | 322 | 11.831 | 13.746 | 57.295 | 1.00 | 56.60 | B |
| ATOM | 6175 | CB | TYR | B | 322 | 10.810 | 14.607 | 58.039 | 1.00 | 42.87 | B |
| ATOM | 6176 | CG | TYR | B | 322 | 10.977 | 16.095 | 57.864 | 1.00 | 33.58 | B |
| ATOM | 6177 | CD1 | TYR | B | 322 | 10.675 | 16.709 | 56.655 | 1.00 | 33.58 | B |
| ATOM | 6178 | CE1 | TYR | B | 322 | 10.776 | 18.091 | 56.494 | 1.00 | 33.58 | B |
| ATOM | 6179 | CD2 | TYR | B | 322 | 11.397 | 16.898 | 58.920 | 1.00 | 33.58 | B |
| ATOM | 6180 | CE2 | TYR | B | 322 | 11.499 | 18.274 | 58.776 | 1.00 | 33.58 | B |
| ATOM | 6181 | CZ | TYR | B | 322 | 11.182 | 18.870 | 57.555 | 1.00 | 33.58 | B |
| ATOM | 6182 | OH | TYR | B | 322 | 11.238 | 20.242 | 57.394 | 1.00 | 33.58 | B |
| ATOM | 6183 | C | TYR | B | 322 | 11.791 | 12.339 | 57.870 | 1.00 | 56.60 | B |
| ATOM | 6184 | O | TYR | B | 322 | 12.690 | 11.914 | 58.602 | 1.00 | 56.60 | B |
| ATOM | 6185 | N | GLN | B | 323 | 10.732 | 11.617 | 57.531 | 1.00 | 49.55 | B |
| ATOM | 6186 | CA | GLN | B | 323 | 10.563 | 10.258 | 58.006 | 1.00 | 49.55 | B |
| ATOM | 6187 | CB | GLN | B | 323 | 9.337 | 9.636 | 57.368 | 1.00 | 71.56 | B |
| ATOM | 6188 | CG | GLN | B | 323 | 9.098 | 8.228 | 57.823 | 1.00 | 45.95 | B |
| ATOM | 6189 | CD | GLN | B | 323 | 7.812 | 7.666 | 57.283 | 1.00 | 45.95 | B |
| ATOM | 6190 | OE1 | GLN | B | 323 | 7.532 | 7.755 | 56.075 | 1.00 | 45.95 | B |
| ATOM | 6191 | NE2 | GLN | B | 323 | 7.014 | 7.077 | 58.166 | 1.00 | 45.95 | B |
| ATOM | 6192 | C | GLN | B | 323 | 11.786 | 9.397 | 57.710 | 1.00 | 49.55 | B |
| ATOM | 6193 | O | GLN | B | 323 | 12.258 | 8.670 | 58.582 | 1.00 | 49.55 | B |
| ATOM | 6194 | N | LYS | B | 324 | 12.299 | 9.471 | 56.483 | 1.00 | 53.99 | B |
| ATOM | 6195 | CA | LYS | B | 324 | 13.484 | 8.702 | 56.113 | 1.00 | 53.99 | B |
| ATOM | 6196 | CB | LYS | B | 324 | 13.911 | 9.015 | 54.680 | 1.00 | 73.94 | B |
| ATOM | 6197 | CG | LYS | B | 324 | 13.089 | 8.362 | 53.607 | 1.00 | 63.37 | B |
| ATOM | 6198 | CD | LYS | B | 324 | 13.768 | 8.516 | 52.251 | 1.00 | 63.37 | B |
| ATOM | 6199 | CE | LYS | B | 324 | 12.994 | 7.800 | 51.148 | 1.00 | 63.37 | B |
| ATOM | 6200 | NZ | LYS | B | 324 | 13.721 | 7.813 | 49.839 | 1.00 | 63.37 | B |
| ATOM | 6201 | C | LYS | B | 324 | 14.670 | 9.001 | 57.032 | 1.00 | 53.99 | B |
| ATOM | 6202 | O | LYS | B | 324 | 15.285 | 8.092 | 57.582 | 1.00 | 53.99 | B |
| ATOM | 6203 | N | MET | B | 325 | 14.995 | 10.280 | 57.193 | 1.00 | 49.43 | B |
| ATOM | 6204 | CA | MET | B | 325 | 16.129 | 10.662 | 58.013 | 1.00 | 49.43 | B |
| ATOM | 6205 | CB | MET | B | 325 | 16.519 | 12.103 | 57.719 | 1.00 | 63.99 | B |
| ATOM | 6206 | CG | MET | B | 325 | 16.977 | 12.305 | 56.312 | 1.00 | 52.55 | B |
| ATOM | 6207 | SD | MET | B | 325 | 17.403 | 13.991 | 55.996 | 1.00 | 52.55 | B |
| ATOM | 6208 | CE | MET | B | 325 | 15.957 | 14.576 | 55.191 | 1.00 | 52.55 | B |
| ATOM | 6209 | C | MET | B | 325 | 15.888 | 10.494 | 59.495 | 1.00 | 49.43 | B |
| ATOM | 6210 | O | MET | B | 325 | 16.828 | 10.519 | 60.291 | 1.00 | 49.43 | B |
| ATOM | 6211 | N | ALA | B | 326 | 14.636 | 10.317 | 59.882 | 1.00 | 49.61 | B |
| ATOM | 6212 | CA | ALA | B | 326 | 14.335 | 10.157 | 61.301 | 1.00 | 49.61 | B |
| ATOM | 6213 | CB | ALA | B | 326 | 12.972 | 10.812 | 61.641 | 1.00 | 29.77 | B |
| ATOM | 6214 | C | ALA | B | 326 | 14.323 | 8.694 | 61.722 | 1.00 | 49.61 | B |
| ATOM | 6215 | O | ALA | B | 326 | 14.596 | 8.383 | 62.875 | 1.00 | 49.61 | B |
| ATOM | 6216 | N | LYS | B | 327 | 14.021 | 7.811 | 60.772 | 1.00 | 74.93 | B |
| ATOM | 6217 | CA | LYS | B | 327 | 13.917 | 6.382 | 61.030 | 1.00 | 74.93 | B |
| ATOM | 6218 | CB | LYS | B | 327 | 14.427 | 5.569 | 59.841 | 1.00 | 153.80 | B |
| ATOM | 6219 | CG | LYS | B | 327 | 14.025 | 4.106 | 59.946 | 1.00 | 72.09 | B |
| ATOM | 6220 | CD | LYS | B | 327 | 14.407 | 3.309 | 58.712 | 1.00 | 72.09 | B |
| ATOM | 6221 | CE | LYS | B | 327 | 13.906 | 1.863 | 58.806 | 1.00 | 72.09 | B |
| ATOM | 6222 | NZ | LYS | B | 327 | 14.300 | 1.051 | 57.615 | 1.00 | 72.09 | B |
| ATOM | 6223 | C | LYS | B | 327 | 14.587 | 5.875 | 62.302 | 1.00 | 74.93 | B |
| ATOM | 6224 | O | LYS | B | 327 | 13.912 | 5.453 | 63.243 | 1.00 | 74.93 | B |
| ATOM | 6225 | N | GLY | B | 328 | 15.906 | 5.918 | 62.362 | 1.00 | 48.57 | B |
| ATOM | 6226 | CA | GLY | B | 328 | 16.539 | 5.406 | 63.561 | 1.00 | 48.57 | B |
| ATOM | 6227 | C | GLY | B | 328 | 16.747 | 6.345 | 64.745 | 1.00 | 48.57 | B |
| ATOM | 6228 | O | GLY | B | 328 | 16.841 | 5.879 | 65.880 | 1.00 | 48.57 | B |
| ATOM | 6229 | N | LYS | B | 329 | 16.798 | 7.659 | 64.504 | 1.00 | 61.07 | B |
| ATOM | 6230 | CA | LYS | B | 329 | 17.061 | 8.600 | 65.575 | 1.00 | 61.07 | B |
| ATOM | 6231 | CB | LYS | B | 329 | 17.884 | 9.764 | 65.020 | 1.00 | 190.58 | B |
| ATOM | 6232 | CG | LYS | B | 329 | 19.267 | 9.350 | 64.542 | 1.00 | 190.58 | B |
| ATOM | 6233 | CD | LYS | B | 329 | 20.178 | 10.556 | 64.440 | 1.00 | 190.58 | B |
| ATOM | 6234 | CE | LYS | B | 329 | 21.600 | 10.179 | 64.063 | 1.00 | 80.34 | B |
| ATOM | 6235 | NZ | LYS | B | 329 | 22.466 | 11.401 | 64.021 | 1.00 | 80.34 | B |
| ATOM | 6236 | C | LYS | B | 329 | 15.935 | 9.147 | 66.448 | 1.00 | 61.07 | B |
| ATOM | 6237 | O | LYS | B | 329 | 16.144 | 10.137 | 67.134 | 1.00 | 61.07 | B |
| ATOM | 6238 | N | ILE | B | 330 | 14.754 | 8.538 | 66.456 | 1.00 | 48.24 | B |
| ATOM | 6239 | CA | ILE | B | 330 | 13.682 | 9.049 | 67.325 | 1.00 | 48.24 | B |
| ATOM | 6240 | CB | ILE | B | 330 | 12.668 | 9.957 | 66.557 | 1.00 | 41.26 | B |
| ATOM | 6241 | CG2 | ILE | B | 330 | 13.404 | 11.102 | 65.864 | 1.00 | 42.18 | B |
| ATOM | 6242 | CG1 | ILE | B | 330 | 11.871 | 9.134 | 65.539 | 1.00 | 42.18 | B |
| ATOM | 6243 | CD1 | ILE | B | 330 | 10.647 | 9.828 | 65.047 | 1.00 | 42.18 | B |
| ATOM | 6244 | C | ILE | B | 330 | 12.918 | 7.895 | 67.962 | 1.00 | 48.24 | B |
| ATOM | 6245 | O | ILE | B | 330 | 13.102 | 6.747 | 67.566 | 1.00 | 48.24 | B |
| ATOM | 6246 | N | PRO | B | 331 | 12.047 | 8.177 | 68.955 | 1.00 | 48.99 | B |
| ATOM | 6247 | CD | PRO | B | 331 | 11.670 | 9.497 | 69.477 | 1.00 | 40.27 | B |
| ATOM | 6248 | CA | PRO | B | 331 | 11.264 | 7.133 | 69.630 | 1.00 | 48.99 | B |
| ATOM | 6249 | CB | PRO | B | 331 | 10.191 | 7.925 | 70.354 | 1.00 | 184.25 | B |
| ATOM | 6250 | CG | PRO | B | 331 | 10.907 | 9.116 | 70.733 | 1.00 | 40.27 | B |
| ATOM | 6251 | C | PRO | B | 331 | 10.663 | 6.156 | 68.635 | 1.00 | 48.99 | B |
| ATOM | 6252 | O | PRO | B | 331 | 10.129 | 6.564 | 67.602 | 1.00 | 48.99 | B |
| ATOM | 6253 | N | LYS | B | 332 | 10.761 | 4.868 | 68.944 | 1.00 | 52.71 | B |
| ATOM | 6254 | CA | LYS | B | 332 | 10.214 | 3.829 | 68.081 | 1.00 | 53.33 | B |
| ATOM | 6255 | CB | LYS | B | 332 | 10.725 | 2.463 | 68.521 | 1.00 | 120.91 | B |
| ATOM | 6256 | CG | LYS | B | 332 | 10.311 | 2.117 | 69.930 | 1.00 | 92.45 | B |
| ATOM | 6257 | CD | LYS | B | 332 | 10.396 | 0.627 | 70.183 | 1.00 | 92.45 | B |
| ATOM | 6258 | CE | LYS | B | 332 | 9.775 | 0.269 | 71.524 | 1.00 | 92.45 | B |
| ATOM | 6259 | NZ | LYS | B | 332 | 9.750 | −1.200 | 71.738 | 1.00 | 92.45 | B |
| ATOM | 6260 | C | LYS | B | 332 | 8.692 | 3.842 | 68.170 | 1.00 | 52.40 | B |
| ATOM | 6261 | O | LYS | B | 332 | 8.123 | 4.328 | 69.152 | 1.00 | 50.49 | B |
| ATOM | 6262 | N | ASP | B | 333 | 8.033 | 3.316 | 67.140 | 1.00 | 54.99 | B |
| ATOM | 6263 | CA | ASP | B | 333 | 6.573 | 3.247 | 67.127 | 1.00 | 54.45 | B |
| ATOM | 6264 | CB | ASP | B | 333 | 6.080 | 2.668 | 65.800 | 1.00 | 147.91 | B |
| ATOM | 6265 | CG | ASP | B | 333 | 5.855 | 3.732 | 64.748 | 1.00 | 115.50 | B |
| ATOM | 6266 | OD1 | ASP | B | 333 | 6.648 | 4.696 | 64.696 | 1.00 | 115.50 | B |
| ATOM | 6267 | OD2 | ASP | B | 333 | 4.890 | 3.601 | 63.965 | 1.00 | 115.50 | B |
| ATOM | 6268 | C | ASP | B | 333 | 6.099 | 2.366 | 68.278 | 1.00 | 58.30 | B |
| ATOM | 6269 | O | ASP | B | 333 | 6.717 | 1.355 | 68.602 | 1.00 | 57.95 | B |
| ATOM | 6270 | N | THR | B | 334 | 5.003 | 2.763 | 68.903 | 1.00 | 52.93 | B |
| ATOM | 6271 | CA | THR | B | 334 | 4.468 | 1.999 | 70.010 | 1.00 | 52.65 | B |
| ATOM | 6272 | CB | THR | B | 334 | 4.867 | 2.635 | 71.348 | 1.00 | 73.63 | B |
| ATOM | 6273 | OG1 | THR | B | 334 | 6.251 | 2.357 | 71.598 | 1.00 | 59.53 | B |
| ATOM | 6274 | CG2 | THR | B | 334 | 4.021 | 2.090 | 72.483 | 1.00 | 59.53 | B |
| ATOM | 6275 | C | THR | B | 334 | 2.958 | 1.912 | 69.912 | 1.00 | 59.09 | B |
| ATOM | 6276 | O | THR | B | 334 | 2.262 | 2.934 | 69.916 | 1.00 | 56.45 | B |
| ATOM | 6277 | N | THR | B | 335 | 2.458 | 0.681 | 69.825 | 1.00 | 44.32 | B |
| ATOM | 6278 | CA | THR | B | 335 | 1.021 | 0.438 | 69.716 | 1.00 | 44.11 | B |
| ATOM | 6279 | CB | THR | B | 335 | 0.750 | −0.965 | 69.222 | 1.00 | 57.94 | B |
| ATOM | 6280 | OG1 | THR | B | 335 | 1.603 | −1.251 | 68.106 | 1.00 | 47.30 | B |
| ATOM | 6281 | CG2 | THR | B | 335 | −0.691 | −1.082 | 68.796 | 1.00 | 47.30 | B |
| ATOM | 6282 | C | THR | B | 335 | 0.278 | 0.630 | 71.027 | 1.00 | 44.97 | B |
| ATOM | 6283 | O | THR | B | 335 | 0.673 | 0.115 | 72.062 | 1.00 | 46.32 | B |
| ATOM | 6284 | N | PRO | B | 336 | −0.834 | 1.357 | 70.991 | 1.00 | 57.80 | B |
| ATOM | 6285 | CD | PRO | B | 336 | −1.464 | 1.994 | 69.823 | 1.00 | 42.82 | B |
| ATOM | 6286 | CA | PRO | B | 336 | −1.612 | 1.608 | 72.200 | 1.00 | 57.38 | B |
| ATOM | 6287 | CB | PRO | B | 336 | −2.375 | 2.862 | 71.840 | 1.00 | 51.68 | B |
| ATOM | 6288 | CG | PRO | B | 336 | −2.763 | 2.549 | 70.411 | 1.00 | 42.82 | B |
| ATOM | 6289 | C | PRO | B | 336 | −2.562 | 0.479 | 72.598 | 1.00 | 57.42 | B |
| ATOM | 6290 | O | PRO | B | 336 | −2.719 | −0.525 | 71.894 | 1.00 | 58.05 | B |
| ATOM | 6291 | N | THR | B | 337 | −3.189 | 0.681 | 73.749 | 1.00 | 62.94 | B |
| ATOM | 6292 | CA | THR | B | 337 | −4.165 | −0.227 | 74.304 | 1.00 | 66.40 | B |
| ATOM | 6293 | CB | THR | B | 337 | −3.812 | −0.570 | 75.745 | 1.00 | 131.09 | B |
| ATOM | 6294 | OG1 | THR | B | 337 | −2.675 | −1.432 | 75.747 | 1.00 | 81.98 | B |
| ATOM | 6295 | CG2 | THR | B | 337 | −4.975 | −1.241 | 76.446 | 1.00 | 81.98 | B |
| ATOM | 6296 | C | THR | B | 337 | −5.447 | 0.588 | 74.290 | 1.00 | 73.97 | B |
| ATOM | 6297 | O | THR | B | 337 | −5.454 | 1.744 | 74.715 | 1.00 | 65.35 | B |
| ATOM | 6298 | N | VAL | B | 338 | −6.531 | 0.012 | 73.790 | 1.00 | 57.88 | B |
| ATOM | 6299 | CA | VAL | B | 338 | −7.779 | 0.758 | 73.753 | 1.00 | 64.74 | B |
| ATOM | 6300 | CB | VAL | B | 338 | −8.266 | 0.977 | 72.295 | 1.00 | 80.93 | B |
| ATOM | 6301 | CG1 | VAL | B | 338 | −9.421 | 1.983 | 72.275 | 1.00 | 80.55 | B |
| ATOM | 6302 | CG2 | VAL | B | 338 | −7.118 | 1.460 | 71.427 | 1.00 | 80.55 | B |
| ATOM | 6303 | C | VAL | B | 338 | −8.879 | 0.065 | 74.546 | 1.00 | 69.21 | B |
| ATOM | 6304 | O | VAL | B | 338 | −8.928 | −1.162 | 74.635 | 1.00 | 69.04 | B |
| ATOM | 6305 | N | LYS | B | 339 | −9.758 | 0.875 | 75.123 | 1.00 | 50.43 | B |
| ATOM | 6306 | CA | LYS | B | 339 | −10.878 | 0.397 | 75.912 | 1.00 | 48.31 | B |
| ATOM | 6307 | CB | LYS | B | 339 | −10.439 | 0.170 | 77.360 | 1.00 | 142.02 | B |
| ATOM | 6308 | CG | LYS | B | 339 | −9.407 | −0.932 | 77.516 | 1.00 | 110.33 | B |
| ATOM | 6309 | CD | LYS | B | 339 | −8.448 | −0.660 | 78.669 | 1.00 | 110.33 | B |
| ATOM | 6310 | CE | LYS | B | 339 | −7.331 | −1.697 | 78.709 | 1.00 | 110.33 | B |
| ATOM | 6311 | NZ | LYS | B | 339 | −6.267 | −1.344 | 79.690 | 1.00 | 110.33 | B |
| ATOM | 6312 | C | LYS | B | 339 | −11.925 | 1.491 | 75.853 | 1.00 | 57.30 | B |
| ATOM | 6313 | O | LYS | B | 339 | −11.615 | 2.668 | 76.042 | 1.00 | 49.96 | B |
| ATOM | 6314 | N | VAL | B | 340 | −13.162 | 1.111 | 75.569 | 1.00 | 84.19 | B |
| ATOM | 6315 | CA | VAL | B | 340 | −14.245 | 2.085 | 75.508 | 1.00 | 81.62 | B |
| ATOM | 6316 | CB | VAL | B | 340 | −15.105 | 1.898 | 74.251 | 1.00 | 58.49 | B |
| ATOM | 6317 | CG1 | VAL | B | 340 | −16.219 | 2.929 | 74.235 | 1.00 | 43.95 | B |
| ATOM | 6318 | CG2 | VAL | B | 340 | −14.248 | 2.021 | 73.010 | 1.00 | 43.95 | B |
| ATOM | 6319 | C | VAL | B | 340 | −15.137 | 1.866 | 76.720 | 1.00 | 84.19 | B |
| ATOM | 6320 | O | VAL | B | 340 | −15.619 | 0.765 | 76.921 | 1.00 | 81.04 | B |
| ATOM | 6321 | N | THR | B | 341 | −15.364 | 2.900 | 77.525 | 1.00 | 85.17 | B |
| ATOM | 6322 | CA | THR | B | 341 | −16.204 | 2.746 | 78.707 | 1.00 | 81.07 | B |
| ATOM | 6323 | CB | THR | B | 341 | −15.474 | 3.230 | 79.969 | 1.00 | 101.04 | B |
| ATOM | 6324 | OG1 | THR | B | 341 | −14.275 | 2.468 | 80.143 | 1.00 | 82.40 | B |
| ATOM | 6325 | CG2 | THR | B | 341 | −16.359 | 3.057 | 81.198 | 1.00 | 82.40 | B |
| ATOM | 6326 | C | THR | B | 341 | −17.521 | 3.497 | 78.589 | 1.00 | 83.22 | B |
| ATOM | 6327 | O | THR | B | 341 | −17.583 | 4.583 | 78.014 | 1.00 | 84.82 | B |
| ATOM | 6328 | N | GLY | B | 342 | −18.572 | 2.913 | 79.150 | 1.00 | 147.83 | B |
| ATOM | 6329 | CA | GLY | B | 342 | −19.873 | 3.546 | 79.094 | 1.00 | 152.00 | B |
| ATOM | 6330 | C | GLY | B | 342 | −20.324 | 4.094 | 80.432 | 1.00 | 150.97 | B |
| ATOM | 6331 | O | GLY | B | 342 | −21.305 | 3.615 | 81.000 | 1.00 | 145.24 | B |
| ATOM | 6332 | N | ASN | B | 343 | −19.609 | 5.092 | 80.942 | 1.00 | 87.50 | B |
| ATOM | 6333 | CA | ASN | B | 343 | −19.963 | 5.714 | 82.216 | 1.00 | 93.91 | B |
| ATOM | 6334 | CB | ASN | B | 343 | −18.874 | 6.688 | 82.661 | 1.00 | 176.87 | B |
| ATOM | 6335 | CG | ASN | B | 343 | −17.499 | 6.058 | 82.681 | 1.00 | 156.67 | B |
| ATOM | 6336 | OD1 | ASN | B | 343 | −17.246 | 5.109 | 83.423 | 1.00 | 156.67 | B |
| ATOM | 6337 | ND2 | ASN | B | 343 | −16.598 | 6.587 | 81.861 | 1.00 | 156.67 | B |
| ATOM | 6338 | C | ASN | B | 343 | −21.259 | 6.486 | 82.014 | 1.00 | 91.97 | B |
| ATOM | 6339 | O | ASN | B | 343 | −21.932 | 6.303 | 81.007 | 1.00 | 80.60 | B |
| ATOM | 6340 | N | GLU | B | 344 | −21.605 | 7.348 | 82.966 | 1.00 | 168.21 | B |
| ATOM | 6341 | CA | GLU | B | 344 | −22.818 | 8.156 | 82.860 | 1.00 | 168.21 | B |
| ATOM | 6342 | CB | GLU | B | 344 | −24.058 | 7.299 | 83.143 | 1.00 | 200.10 | B |
| ATOM | 6343 | CG | GLU | B | 344 | −24.604 | 6.633 | 81.881 | 1.00 | 180.00 | B |
| ATOM | 6344 | CD | GLU | B | 344 | −25.718 | 5.643 | 82.148 | 1.00 | 180.00 | B |
| ATOM | 6345 | OE1 | GLU | B | 344 | −25.429 | 4.538 | 82.651 | 1.00 | 180.00 | B |
| ATOM | 6346 | OE2 | GLU | B | 344 | −26.886 | 5.971 | 81.855 | 1.00 | 180.00 | B |
| ATOM | 6347 | C | GLU | B | 344 | −22.757 | 9.367 | 83.785 | 1.00 | 168.21 | B |
| ATOM | 6348 | O | GLU | B | 344 | −22.844 | 9.245 | 85.006 | 1.00 | 168.21 | B |
| ATOM | 6349 | N | LEU | B | 345 | −22.608 | 10.538 | 83.170 | 1.00 | 200.84 | B |
| ATOM | 6350 | CA | LEU | B | 345 | −22.481 | 11.806 | 83.882 | 1.00 | 200.84 | B |
| ATOM | 6351 | CB | LEU | B | 345 | −21.903 | 12.869 | 82.934 | 1.00 | 151.36 | B |
| ATOM | 6352 | CG | LEU | B | 345 | −20.510 | 12.605 | 82.337 | 1.00 | 104.15 | B |
| ATOM | 6353 | CD1 | LEU | B | 345 | −20.080 | 13.787 | 81.477 | 1.00 | 104.15 | B |
| ATOM | 6354 | CD2 | LEU | B | 345 | −19.497 | 12.381 | 83.447 | 1.00 | 104.15 | B |
| ATOM | 6355 | C | LEU | B | 345 | −23.723 | 12.363 | 84.591 | 1.00 | 200.84 | B |
| ATOM | 6356 | O | LEU | B | 345 | −23.913 | 13.578 | 84.660 | 1.00 | 200.84 | B |
| ATOM | 6357 | N | GLU | B | 346 | −24.560 | 11.471 | 85.112 | 1.00 | 200.84 | B |
| ATOM | 6358 | CA | GLU | B | 346 | −25.757 | 11.842 | 85.873 | 1.00 | 200.84 | B |
| ATOM | 6359 | CB | GLU | B | 346 | −25.327 | 12.611 | 87.124 | 1.00 | 168.29 | B |
| ATOM | 6360 | CG | GLU | B | 346 | −24.112 | 11.985 | 87.793 | 1.00 | 153.72 | B |
| ATOM | 6361 | CD | GLU | B | 346 | −23.943 | 12.399 | 89.236 | 1.00 | 153.72 | B |
| ATOM | 6362 | OE1 | GLU | B | 346 | −24.806 | 12.029 | 90.061 | 1.00 | 153.72 | B |
| ATOM | 6363 | OE2 | GLU | B | 346 | −22.948 | 13.091 | 89.543 | 1.00 | 153.72 | B |
| ATOM | 6364 | C | GLU | B | 346 | −26.910 | 12.585 | 85.186 | 1.00 | 200.84 | B |
| ATOM | 6365 | O | GLU | B | 346 | −27.958 | 11.989 | 84.928 | 1.00 | 200.84 | B |
| ATOM | 6366 | N | ASP | B | 347 | −26.741 | 13.876 | 84.905 | 1.00 | 200.84 | B |
| ATOM | 6367 | CA | ASP | B | 347 | −27.813 | 14.647 | 84.273 | 1.00 | 200.84 | B |
| ATOM | 6368 | CB | ASP | B | 347 | −27.425 | 16.128 | 84.153 | 1.00 | 196.76 | B |
| ATOM | 6369 | CG | ASP | B | 347 | −26.568 | 16.416 | 82.933 | 1.00 | 196.76 | B |
| ATOM | 6370 | OD1 | ASP | B | 347 | −25.456 | 15.857 | 82.839 | 1.00 | 196.76 | B |
| ATOM | 6371 | OD2 | ASP | B | 347 | −27.007 | 17.204 | 82.066 | 1.00 | 196.76 | B |
| ATOM | 6372 | C | ASP | B | 347 | −28.194 | 14.100 | 82.897 | 1.00 | 200.84 | B |
| ATOM | 6373 | O | ASP | B | 347 | −29.371 | 14.094 | 82.531 | 1.00 | 200.84 | B |
| ATOM | 6374 | N | GLY | B | 348 | −27.198 | 13.642 | 82.142 | 1.00 | 156.14 | B |
| ATOM | 6375 | CA | GLY | B | 348 | −27.456 | 13.096 | 80.821 | 1.00 | 153.45 | B |
| ATOM | 6376 | C | GLY | B | 348 | −27.524 | 11.583 | 80.854 | 1.00 | 150.41 | B |
| ATOM | 6377 | O | GLY | B | 348 | −26.841 | 10.952 | 81.660 | 1.00 | 145.42 | B |
| ATOM | 6378 | N | ASN | B | 349 | −28.338 | 10.997 | 79.979 | 1.00 | 200.84 | B |
| ATOM | 6379 | CA | ASN | B | 349 | −28.486 | 9.548 | 79.944 | 1.00 | 200.84 | B |
| ATOM | 6380 | CB | ASN | B | 349 | −29.342 | 9.124 | 78.739 | 1.00 | 196.27 | B |
| ATOM | 6381 | CG | ASN | B | 349 | −29.118 | 9.995 | 77.517 | 1.00 | 181.45 | B |
| ATOM | 6382 | OD1 | ASN | B | 349 | −29.374 | 11.200 | 77.540 | 1.00 | 181.45 | B |
| ATOM | 6383 | ND2 | ASN | B | 349 | −28.645 | 9.385 | 76.439 | 1.00 | 181.45 | B |
| ATOM | 6384 | C | ASN | B | 349 | −27.162 | 8.778 | 79.967 | 1.00 | 200.84 | B |
| ATOM | 6385 | O | ASN | B | 349 | −26.713 | 8.362 | 81.034 | 1.00 | 200.84 | B |
| ATOM | 6386 | N | MET | B | 350 | −26.526 | 8.603 | 78.813 | 1.00 | 161.36 | B |
| ATOM | 6387 | CA | MET | B | 350 | −25.272 | 7.851 | 78.758 | 1.00 | 157.45 | B |
| ATOM | 6388 | CB | MET | B | 350 | −25.485 | 6.578 | 77.928 | 1.00 | 200.84 | B |
| ATOM | 6389 | CG | MET | B | 350 | −24.255 | 5.698 | 77.781 | 1.00 | 165.92 | B |
| ATOM | 6390 | SD | MET | B | 350 | −24.603 | 4.149 | 76.920 | 1.00 | 165.92 | B |
| ATOM | 6391 | CE | MET | B | 350 | −24.674 | 3.002 | 78.299 | 1.00 | 165.92 | B |
| ATOM | 6392 | C | MET | B | 350 | −24.067 | 8.629 | 78.216 | 1.00 | 154.59 | B |
| ATOM | 6393 | O | MET | B | 350 | −24.217 | 9.608 | 77.487 | 1.00 | 151.02 | B |
| ATOM | 6394 | N | THR | B | 351 | −22.871 | 8.179 | 78.590 | 1.00 | 105.28 | B |
| ATOM | 6395 | CA | THR | B | 351 | −21.625 | 8.800 | 78.141 | 1.00 | 103.24 | B |
| ATOM | 6396 | CB | THR | B | 351 | −20.999 | 9.687 | 79.252 | 1.00 | 112.68 | B |
| ATOM | 6397 | OG1 | THR | B | 351 | −20.324 | 10.796 | 78.652 | 1.00 | 79.80 | B |
| ATOM | 6398 | CG2 | THR | B | 351 | −19.972 | 8.907 | 80.065 | 1.00 | 79.80 | B |
| ATOM | 6399 | C | THR | B | 351 | −20.605 | 7.727 | 77.735 | 1.00 | 97.47 | B |
| ATOM | 6400 | O | THR | B | 351 | −20.411 | 6.733 | 78.443 | 1.00 | 89.06 | B |
| ATOM | 6401 | N | LEU | B | 352 | −19.961 | 7.925 | 76.590 | 1.00 | 88.85 | B |
| ATOM | 6402 | CA | LEU | B | 352 | −18.962 | 6.975 | 76.121 | 1.00 | 91.87 | B |
| ATOM | 6403 | CB | LEU | B | 352 | −19.281 | 6.528 | 74.695 | 1.00 | 94.05 | B |
| ATOM | 6404 | CG | LEU | B | 352 | −20.369 | 5.455 | 74.658 | 1.00 | 75.11 | B |
| ATOM | 6405 | CD1 | LEU | B | 352 | −20.858 | 5.242 | 73.235 | 1.00 | 75.11 | B |
| ATOM | 6406 | CD2 | LEU | B | 352 | −19.813 | 4.169 | 75.244 | 1.00 | 75.11 | B |
| ATOM | 6407 | C | LEU | B | 352 | −17.566 | 7.572 | 76.194 | 1.00 | 88.55 | B |
| ATOM | 6408 | O | LEU | B | 352 | −17.345 | 8.731 | 75.834 | 1.00 | 84.17 | B |
| ATOM | 6409 | N | GLU | B | 353 | −16.628 | 6.767 | 76.676 | 1.00 | 82.35 | B |
| ATOM | 6410 | CA | GLU | B | 353 | −15.258 | 7.209 | 76.824 | 1.00 | 78.60 | B |
| ATOM | 6411 | CB | GLU | B | 353 | −14.929 | 7.371 | 78.302 | 1.00 | 200.84 | B |
| ATOM | 6412 | CG | GLU | B | 353 | −13.532 | 7.872 | 78.565 | 1.00 | 168.91 | B |
| ATOM | 6413 | CD | GLU | B | 353 | −13.225 | 7.928 | 80.038 | 1.00 | 168.91 | B |
| ATOM | 6414 | OE1 | GLU | B | 353 | −13.972 | 8.609 | 80.772 | 1.00 | 168.91 | B |
| ATOM | 6415 | OE2 | GLU | B | 353 | −12.239 | 7.290 | 80.462 | 1.00 | 168.91 | B |
| ATOM | 6416 | C | GLU | B | 353 | −14.276 | 6.237 | 76.186 | 1.00 | 81.85 | B |
| ATOM | 6417 | O | GLU | B | 353 | −14.133 | 5.093 | 76.625 | 1.00 | 82.63 | B |
| ATOM | 6418 | N | CYS | B | 354 | −13.601 | 6.711 | 75.143 | 1.00 | 82.50 | B |
| ATOM | 6419 | CA | CYS | B | 354 | −12.616 | 5.921 | 74.422 | 1.00 | 82.78 | B |
| ATOM | 6420 | C | CYS | B | 354 | −11.265 | 6.337 | 74.970 | 1.00 | 83.87 | B |
| ATOM | 6421 | O | CYS | B | 354 | −10.921 | 7.517 | 74.929 | 1.00 | 82.57 | B |
| ATOM | 6422 | CB | CYS | B | 354 | −12.684 | 6.249 | 72.932 | 1.00 | 116.09 | B |
| ATOM | 6423 | SG | CYS | B | 354 | −11.841 | 5.084 | 71.810 | 1.00 | 93.28 | B |
| ATOM | 6424 | N | THR | B | 355 | −10.506 | 5.391 | 75.510 | 1.00 | 53.31 | B |
| ATOM | 6425 | CA | THR | B | 355 | −9.194 | 5.742 | 76.031 | 1.00 | 42.67 | B |
| ATOM | 6426 | CB | THR | B | 355 | −9.080 | 5.537 | 77.554 | 1.00 | 92.36 | B |
| ATOM | 6427 | OG1 | THR | B | 355 | −8.380 | 4.320 | 77.808 | 1.00 | 41.51 | B |
| ATOM | 6428 | CG2 | THR | B | 355 | −10.453 | 5.483 | 78.208 | 1.00 | 41.51 | B |
| ATOM | 6429 | C | THR | B | 355 | −8.118 | 4.918 | 75.344 | 1.00 | 56.16 | B |
| ATOM | 6430 | O | THR | B | 355 | −8.186 | 3.685 | 75.292 | 1.00 | 48.96 | B |
| ATOM | 6431 | N | VAL | B | 356 | −7.127 | 5.621 | 74.807 | 1.00 | 62.23 | B |
| ATOM | 6432 | CA | VAL | B | 356 | −6.023 | 4.991 | 74.098 | 1.00 | 57.95 | B |
| ATOM | 6433 | CB | VAL | B | 356 | −5.835 | 5.666 | 72.723 | 1.00 | 64.17 | B |
| ATOM | 6434 | CG1 | VAL | B | 356 | −4.868 | 4.878 | 71.865 | 1.00 | 48.70 | B |
| ATOM | 6435 | CG2 | VAL | B | 356 | −7.187 | 5.772 | 72.029 | 1.00 | 48.70 | B |
| ATOM | 6436 | C | VAL | B | 356 | −4.794 | 5.157 | 74.970 | 1.00 | 66.79 | B |
| ATOM | 6437 | O | VAL | B | 356 | −4.404 | 6.274 | 75.302 | 1.00 | 67.90 | B |
| ATOM | 6438 | N | ASN | B | 357 | −4.186 | 4.036 | 75.334 | 1.00 | 51.54 | B |
| ATOM | 6439 | CA | ASN | B | 357 | −3.036 | 4.052 | 76.227 | 1.00 | 47.76 | B |
| ATOM | 6440 | CB | ASN | B | 357 | −3.245 | 3.057 | 77.362 | 1.00 | 130.73 | B |
| ATOM | 6441 | CG | ASN | B | 357 | −4.413 | 3.427 | 78.234 | 1.00 | 84.65 | B |
| ATOM | 6442 | OD1 | ASN | B | 357 | −4.491 | 4.544 | 78.744 | 1.00 | 84.65 | B |
| ATOM | 6443 | ND2 | ASN | B | 357 | −5.337 | 2.492 | 78.409 | 1.00 | 84.65 | B |
| ATOM | 6444 | C | ASN | B | 357 | −1.666 | 3.832 | 75.654 | 1.00 | 51.92 | B |
| ATOM | 6445 | O | ASN | B | 357 | −1.404 | 2.836 | 74.973 | 1.00 | 54.80 | B |
| ATOM | 6446 | N | SER | B | 358 | −0.793 | 4.778 | 75.998 | 1.00 | 55.92 | B |
| ATOM | 6447 | CA | SER | B | 358 | 0.605 | 4.822 | 75.589 | 1.00 | 63.22 | B |
| ATOM | 6448 | CB | SER | B | 358 | 1.475 | 4.093 | 76.621 | 1.00 | 55.35 | B |
| ATOM | 6449 | OG | SER | B | 358 | 1.231 | 2.711 | 76.574 | 1.00 | 53.69 | B |
| ATOM | 6450 | C | SER | B | 358 | 0.929 | 4.327 | 74.172 | 1.00 | 67.94 | B |
| ATOM | 6451 | O | SER | B | 358 | 1.240 | 3.152 | 73.953 | 1.00 | 63.49 | B |
| ATOM | 6452 | N | PHE | B | 359 | 0.866 | 5.271 | 73.232 | 1.00 | 44.00 | B |
| ATOM | 6453 | CA | PHE | B | 359 | 1.154 | 5.060 | 71.805 | 1.00 | 38.31 | B |
| ATOM | 6454 | CB | PHE | B | 359 | −0.094 | 5.401 | 70.991 | 1.00 | 40.69 | B |
| ATOM | 6455 | CG | PHE | B | 359 | −0.754 | 6.699 | 71.394 | 1.00 | 35.29 | B |
| ATOM | 6456 | CD1 | PHE | B | 359 | −0.296 | 7.911 | 70.903 | 1.00 | 35.29 | B |
| ATOM | 6457 | CD2 | PHE | B | 359 | −1.860 | 6.697 | 72.243 | 1.00 | 35.29 | B |
| ATOM | 6458 | CE1 | PHE | B | 359 | −0.926 | 9.100 | 71.243 | 1.00 | 35.29 | B |
| ATOM | 6459 | CE2 | PHE | B | 359 | −2.503 | 7.884 | 72.592 | 1.00 | 35.29 | B |
| ATOM | 6460 | CZ | PHE | B | 359 | −2.036 | 9.085 | 72.090 | 1.00 | 35.29 | B |
| ATOM | 6461 | C | PHE | B | 359 | 2.298 | 6.011 | 71.445 | 1.00 | 44.90 | B |
| ATOM | 6462 | O | PHE | B | 359 | 2.349 | 7.104 | 72.007 | 1.00 | 42.45 | B |
| ATOM | 6463 | N | TYR | B | 360 | 3.216 | 5.651 | 70.546 | 1.00 | 127.29 | B |
| ATOM | 6464 | CA | TYR | B | 360 | 4.258 | 6.637 | 70.305 | 1.00 | 127.07 | B |
| ATOM | 6465 | CB | TYR | B | 360 | 5.596 | 6.088 | 69.824 | 1.00 | 156.52 | B |
| ATOM | 6466 | CG | TYR | B | 360 | 6.450 | 7.292 | 69.427 | 1.00 | 43.66 | B |
| ATOM | 6467 | CD1 | TYR | B | 360 | 6.535 | 8.416 | 70.270 | 1.00 | 43.66 | B |
| ATOM | 6468 | CE1 | TYR | B | 360 | 7.172 | 9.599 | 69.860 | 1.00 | 43.66 | B |
| ATOM | 6469 | CD2 | TYR | B | 360 | 7.046 | 7.383 | 68.163 | 1.00 | 43.66 | B |
| ATOM | 6470 | CE2 | TYR | B | 360 | 7.697 | 8.564 | 67.749 | 1.00 | 43.66 | B |
| ATOM | 6471 | CZ | TYR | B | 360 | 7.747 | 9.659 | 68.603 | 1.00 | 43.66 | B |
| ATOM | 6472 | OH | TYR | B | 360 | 8.367 | 10.806 | 68.200 | 1.00 | 43.66 | B |
| ATOM | 6473 | C | TYR | B | 360 | 3.957 | 7.805 | 69.403 | 1.00 | 128.16 | B |
| ATOM | 6474 | O | TYR | B | 360 | 3.669 | 8.884 | 69.902 | 1.00 | 134.09 | B |
| ATOM | 6475 | N | PRO | B | 361 | 4.064 | 7.633 | 68.072 | 1.00 | 69.06 | B |
| ATOM | 6476 | CD | PRO | B | 361 | 3.906 | 6.412 | 67.257 | 1.00 | 40.46 | B |
| ATOM | 6477 | CA | PRO | B | 361 | 3.757 | 8.860 | 67.313 | 1.00 | 64.65 | B |
| ATOM | 6478 | CB | PRO | B | 361 | 3.553 | 8.360 | 65.880 | 1.00 | 39.70 | B |
| ATOM | 6479 | CG | PRO | B | 361 | 3.115 | 6.928 | 66.053 | 1.00 | 40.46 | B |
| ATOM | 6480 | C | PRO | B | 361 | 2.525 | 9.474 | 67.984 | 1.00 | 72.05 | B |
| ATOM | 6481 | O | PRO | B | 361 | 1.443 | 8.901 | 67.982 | 1.00 | 69.22 | B |
| ATOM | 6482 | N | PRO | B | 362 | 2.711 | 10.630 | 68.631 | 1.00 | 42.48 | B |
| ATOM | 6483 | CD | PRO | B | 362 | 3.969 | 11.394 | 68.584 | 1.00 | 51.93 | B |
| ATOM | 6484 | CA | PRO | B | 362 | 1.683 | 11.372 | 69.358 | 1.00 | 36.19 | B |
| ATOM | 6485 | CB | PRO | B | 362 | 2.452 | 12.577 | 69.904 | 1.00 | 51.93 | B |
| ATOM | 6486 | CG | PRO | B | 362 | 3.510 | 12.781 | 68.910 | 1.00 | 51.93 | B |
| ATOM | 6487 | C | PRO | B | 362 | 0.398 | 11.756 | 68.652 | 1.00 | 38.79 | B |
| ATOM | 6488 | O | PRO | B | 362 | −0.589 | 12.110 | 69.311 | 1.00 | 43.55 | B |
| ATOM | 6489 | N | ASP | B | 363 | 0.401 | 11.694 | 67.324 | 1.00 | 39.87 | B |
| ATOM | 6490 | CA | ASP | B | 363 | −0.794 | 12.031 | 66.557 | 1.00 | 39.72 | B |
| ATOM | 6491 | CB | ASP | B | 363 | −0.440 | 12.403 | 65.100 | 1.00 | 74.56 | B |
| ATOM | 6492 | CG | ASP | B | 363 | 0.516 | 11.410 | 64.433 | 1.00 | 68.77 | B |
| ATOM | 6493 | OD1 | ASP | B | 363 | 1.630 | 11.202 | 64.959 | 1.00 | 68.77 | B |
| ATOM | 6494 | OD2 | ASP | B | 363 | 0.165 | 10.848 | 63.369 | 1.00 | 68.77 | B |
| ATOM | 6495 | C | ASP | B | 363 | −1.781 | 10.867 | 66.596 | 1.00 | 43.26 | B |
| ATOM | 6496 | O | ASP | B | 363 | −1.447 | 9.728 | 66.255 | 1.00 | 39.76 | B |
| ATOM | 6497 | N | VAL | B | 364 | −2.992 | 11.154 | 67.055 | 1.00 | 47.90 | B |
| ATOM | 6498 | CA | VAL | B | 364 | −4.035 | 10.148 | 67.127 | 1.00 | 50.22 | B |
| ATOM | 6499 | CB | VAL | B | 364 | −4.149 | 9.519 | 68.525 | 1.00 | 73.65 | B |
| ATOM | 6500 | CG1 | VAL | B | 364 | −5.507 | 8.887 | 68.705 | 1.00 | 52.04 | B |
| ATOM | 6501 | CG2 | VAL | B | 364 | −3.111 | 8.449 | 68.686 | 1.00 | 52.04 | B |
| ATOM | 6502 | C | VAL | B | 364 | −5.332 | 10.835 | 66.807 | 1.00 | 57.56 | B |
| ATOM | 6503 | O | VAL | B | 364 | −5.551 | 11.972 | 67.210 | 1.00 | 49.30 | B |
| ATOM | 6504 | N | ILE | B | 365 | −6.183 | 10.156 | 66.055 | 1.00 | 54.18 | B |
| ATOM | 6505 | CA | ILE | B | 365 | −7.470 | 10.718 | 65.716 | 1.00 | 48.23 | B |
| ATOM | 6506 | CB | ILE | B | 365 | −7.535 | 11.168 | 64.256 | 1.00 | 67.79 | B |
| ATOM | 6507 | CG2 | ILE | B | 365 | −8.954 | 11.575 | 63.892 | 1.00 | 44.23 | B |
| ATOM | 6508 | CG1 | ILE | B | 365 | −6.583 | 12.347 | 64.069 | 1.00 | 44.23 | B |
| ATOM | 6509 | CD1 | ILE | B | 365 | −6.600 | 12.957 | 62.679 | 1.00 | 44.23 | B |
| ATOM | 6510 | C | ILE | B | 365 | −8.509 | 9.671 | 66.008 | 1.00 | 54.99 | B |
| ATOM | 6511 | O | ILE | B | 365 | −8.277 | 8.476 | 65.847 | 1.00 | 55.76 | B |
| ATOM | 6512 | N | THR | B | 366 | −9.661 | 10.147 | 66.451 | 1.00 | 47.72 | B |
| ATOM | 6513 | CA | THR | B | 366 | −10.754 | 9.292 | 66.840 | 1.00 | 56.00 | B |
| ATOM | 6514 | CB | THR | B | 366 | −10.929 | 9.415 | 68.357 | 1.00 | 58.57 | B |
| ATOM | 6515 | OG1 | THR | B | 366 | −10.805 | 8.125 | 68.952 | 1.00 | 46.25 | B |
| ATOM | 6516 | CG2 | THR | B | 366 | −12.253 | 10.037 | 68.709 | 1.00 | 46.25 | B |
| ATOM | 6517 | C | THR | B | 366 | −12.046 | 9.651 | 66.130 | 1.00 | 64.98 | B |
| ATOM | 6518 | O | THR | B | 366 | −12.392 | 10.823 | 66.007 | 1.00 | 64.22 | B |
| ATOM | 6519 | N | LYS | B | 367 | −12.748 | 8.628 | 65.660 | 1.00 | 59.00 | B |
| ATOM | 6520 | CA | LYS | B | 367 | −14.033 | 8.810 | 64.988 | 1.00 | 62.20 | B |
| ATOM | 6521 | CB | LYS | B | 367 | −13.961 | 8.402 | 63.515 | 1.00 | 71.41 | B |
| ATOM | 6522 | CG | LYS | B | 367 | −13.187 | 9.363 | 62.654 | 1.00 | 62.42 | B |
| ATOM | 6523 | CD | LYS | B | 367 | −13.349 | 9.070 | 61.178 | 1.00 | 62.42 | B |
| ATOM | 6524 | CE | LYS | B | 367 | −12.743 | 10.200 | 60.360 | 1.00 | 62.42 | B |
| ATOM | 6525 | NZ | LYS | B | 367 | −13.138 | 10.144 | 58.928 | 1.00 | 62.42 | B |
| ATOM | 6526 | C | LYS | B | 367 | −15.097 | 7.970 | 65.671 | 1.00 | 64.90 | B |
| ATOM | 6527 | O | LYS | B | 367 | −14.889 | 6.780 | 65.922 | 1.00 | 65.21 | B |
| ATOM | 6528 | N | TRP | B | 368 | −16.226 | 8.599 | 65.983 | 1.00 | 67.77 | B |
| ATOM | 6529 | CA | TRP | B | 368 | −17.342 | 7.904 | 66.610 | 1.00 | 67.83 | B |
| ATOM | 6530 | CB | TRP | B | 368 | −17.933 | 8.740 | 67.742 | 1.00 | 84.09 | B |
| ATOM | 6531 | CG | TRP | B | 368 | −17.040 | 8.825 | 68.917 | 1.00 | 64.01 | B |
| ATOM | 6532 | CD2 | TRP | B | 368 | −16.950 | 7.882 | 69.990 | 1.00 | 64.01 | B |
| ATOM | 6533 | CE2 | TRP | B | 368 | −15.935 | 8.333 | 70.859 | 1.00 | 64.01 | B |
| ATOM | 6534 | CE3 | TRP | B | 368 | −17.628 | 6.696 | 70.302 | 1.00 | 64.01 | B |
| ATOM | 6535 | CD1 | TRP | B | 368 | −16.107 | 9.783 | 69.170 | 1.00 | 64.01 | B |
| ATOM | 6536 | NE1 | TRP | B | 368 | −15.436 | 9.496 | 70.337 | 1.00 | 64.01 | B |
| ATOM | 6537 | CZ2 | TRP | B | 368 | −15.582 | 7.641 | 72.019 | 1.00 | 64.01 | B |
| ATOM | 6538 | CZ3 | TRP | B | 368 | −17.275 | 6.008 | 71.455 | 1.00 | 64.01 | B |
| ATOM | 6539 | CH2 | TRP | B | 368 | −16.262 | 6.482 | 72.299 | 1.00 | 64.01 | B |
| ATOM | 6540 | C | TRP | B | 368 | −18.399 | 7.651 | 65.544 | 1.00 | 69.78 | B |
| ATOM | 6541 | O | TRP | B | 368 | −18.962 | 8.590 | 64.975 | 1.00 | 69.97 | B |
| ATOM | 6542 | N | ILE | B | 369 | −18.663 | 6.376 | 65.280 | 1.00 | 77.52 | B |
| ATOM | 6543 | CA | ILE | B | 369 | −19.632 | 5.994 | 64.264 | 1.00 | 78.89 | B |
| ATOM | 6544 | CB | ILE | B | 369 | −18.915 | 5.316 | 63.084 | 1.00 | 86.97 | B |
| ATOM | 6545 | CG2 | ILE | B | 369 | −19.906 | 5.009 | 61.981 | 1.00 | 64.65 | B |
| ATOM | 6546 | CG1 | ILE | B | 369 | −17.803 | 6.230 | 62.567 | 1.00 | 64.65 | B |
| ATOM | 6547 | CD1 | ILE | B | 369 | −16.924 | 5.594 | 61.533 | 1.00 | 64.65 | B |
| ATOM | 6548 | C | ILE | B | 369 | −20.674 | 5.031 | 64.821 | 1.00 | 80.71 | B |
| ATOM | 6549 | O | ILE | B | 369 | −20.329 | 4.085 | 65.531 | 1.00 | 77.51 | B |
| ATOM | 6550 | N | GLU | B | 370 | −21.944 | 5.274 | 64.507 | 1.00 | 59.64 | B |
| ATOM | 6551 | CA | GLU | B | 370 | −23.010 | 4.391 | 64.966 | 1.00 | 62.29 | B |
| ATOM | 6552 | CB | GLU | B | 370 | −24.357 | 4.920 | 64.483 | 1.00 | 115.77 | B |
| ATOM | 6553 | CG | GLU | B | 370 | −24.815 | 6.113 | 65.300 | 1.00 | 94.41 | B |
| ATOM | 6554 | CD | GLU | B | 370 | −26.010 | 6.821 | 64.708 | 1.00 | 94.41 | B |
| ATOM | 6555 | OE1 | GLU | B | 370 | −26.990 | 6.136 | 64.340 | 1.00 | 94.41 | B |
| ATOM | 6556 | OE2 | GLU | B | 370 | −25.974 | 8.067 | 64.623 | 1.00 | 94.41 | B |
| ATOM | 6557 | C | GLU | B | 370 | −22.739 | 2.984 | 64.436 | 1.00 | 62.47 | B |
| ATOM | 6558 | O | GLU | B | 370 | −22.460 | 2.804 | 63.254 | 1.00 | 61.32 | B |
| ATOM | 6559 | N | SER | B | 371 | −22.812 | 1.991 | 65.317 | 1.00 | 76.68 | B |
| ATOM | 6560 | CA | SER | B | 371 | −22.530 | 0.604 | 64.948 | 1.00 | 77.22 | B |
| ATOM | 6561 | CB | SER | B | 371 | −22.675 | −0.295 | 66.175 | 1.00 | 113.66 | B |
| ATOM | 6562 | OG | SER | B | 371 | −23.979 | −0.193 | 66.712 | 1.00 | 102.74 | B |
| ATOM | 6563 | C | SER | B | 371 | −23.350 | 0.016 | 63.794 | 1.00 | 76.49 | B |
| ATOM | 6564 | O | SER | B | 371 | −22.912 | −0.948 | 63.152 | 1.00 | 77.43 | B |
| ATOM | 6565 | N | GLU | B | 372 | −24.533 | 0.573 | 63.538 | 1.00 | 67.40 | B |
| ATOM | 6566 | CA | GLU | B | 372 | −25.366 | 0.074 | 62.456 | 1.00 | 65.95 | B |
| ATOM | 6567 | CB | GLU | B | 372 | −26.623 | 0.930 | 62.312 | 1.00 | 116.02 | B |
| ATOM | 6568 | CG | GLU | B | 372 | −26.339 | 2.396 | 62.066 | 1.00 | 116.02 | B |
| ATOM | 6569 | CD | GLU | B | 372 | −27.565 | 3.161 | 61.599 | 1.00 | 116.02 | B |
| ATOM | 6570 | OE1 | GLU | B | 372 | −28.091 | 2.832 | 60.513 | 1.00 | 116.02 | B |
| ATOM | 6571 | OE2 | GLU | B | 372 | −28.003 | 4.090 | 62.315 | 1.00 | 116.02 | B |
| ATOM | 6572 | C | GLU | B | 372 | −24.578 | 0.091 | 61.141 | 1.00 | 70.56 | B |
| ATOM | 6573 | O | GLU | B | 372 | −24.665 | −0.843 | 60.334 | 1.00 | 71.63 | B |
| ATOM | 6574 | N | HIS | B | 373 | −23.794 | 1.147 | 60.946 | 1.00 | 74.84 | B |
| ATOM | 6575 | CA | HIS | B | 373 | −23.006 | 1.315 | 59.737 | 1.00 | 78.33 | B |
| ATOM | 6576 | CB | HIS | B | 373 | −22.368 | 2.694 | 59.730 | 1.00 | 101.96 | B |
| ATOM | 6577 | CG | HIS | B | 373 | −23.363 | 3.806 | 59.735 | 1.00 | 71.31 | B |
| ATOM | 6578 | CD2 | HIS | B | 373 | −23.522 | 4.859 | 60.571 | 1.00 | 71.31 | B |
| ATOM | 6579 | ND1 | HIS | B | 373 | −24.355 | 3.917 | 58.786 | 1.00 | 71.31 | B |
| ATOM | 6580 | CE1 | HIS | B | 373 | −25.081 | 4.992 | 59.035 | 1.00 | 71.31 | B |
| ATOM | 6581 | NE2 | HIS | B | 373 | −24.597 | 5.583 | 60.113 | 1.00 | 71.31 | B |
| ATOM | 6582 | C | HIS | B | 373 | −21.940 | 0.266 | 59.492 | 1.00 | 74.10 | B |
| ATOM | 6583 | O | HIS | B | 373 | −21.334 | 0.246 | 58.424 | 1.00 | 69.94 | B |
| ATOM | 6584 | N | PHE | B | 374 | −21.695 | −0.608 | 60.459 | 1.00 | 68.01 | B |
| ATOM | 6585 | CA | PHE | B | 374 | −20.689 | −1.637 | 60.249 | 1.00 | 68.78 | B |
| ATOM | 6586 | CB | PHE | B | 374 | −19.813 | −1.817 | 61.484 | 1.00 | 86.00 | B |
| ATOM | 6587 | CG | PHE | B | 374 | −18.865 | −0.692 | 61.698 | 1.00 | 66.18 | B |
| ATOM | 6588 | CD1 | PHE | B | 374 | −19.292 | 0.491 | 62.303 | 1.00 | 66.18 | B |
| ATOM | 6589 | CD2 | PHE | B | 374 | −17.562 | −0.778 | 61.225 | 1.00 | 66.18 | B |
| ATOM | 6590 | CE1 | PHE | B | 374 | −18.435 | 1.576 | 62.426 | 1.00 | 66.18 | B |
| ATOM | 6591 | CE2 | PHE | B | 374 | −16.692 | 0.300 | 61.340 | 1.00 | 66.18 | B |
| ATOM | 6592 | CZ | PHE | B | 374 | −17.129 | 1.482 | 61.942 | 1.00 | 66.18 | B |
| ATOM | 6593 | C | PHE | B | 374 | −21.314 | −2.957 | 59.861 | 1.00 | 64.86 | B |
| ATOM | 6594 | O | PHE | B | 374 | −20.631 | −3.989 | 59.836 | 1.00 | 63.10 | B |
| ATOM | 6595 | N | LYS | B | 375 | −22.610 | −2.909 | 59.546 | 1.00 | 91.94 | B |
| ATOM | 6596 | CA | LYS | B | 375 | −23.379 | −4.082 | 59.131 | 1.00 | 94.57 | B |
| ATOM | 6597 | CB | LYS | B | 375 | −23.474 | −4.139 | 57.598 | 1.00 | 117.62 | B |
| ATOM | 6598 | CG | LYS | B | 375 | −24.526 | −3.225 | 56.962 | 1.00 | 91.46 | B |
| ATOM | 6599 | CD | LYS | B | 375 | −24.165 | −1.748 | 57.045 | 1.00 | 91.46 | B |
| ATOM | 6600 | CE | LYS | B | 375 | −25.200 | −0.887 | 56.320 | 1.00 | 91.46 | B |
| ATOM | 6601 | NZ | LYS | B | 375 | −24.923 | 0.573 | 56.441 | 1.00 | 91.46 | B |
| ATOM | 6602 | C | LYS | B | 375 | −22.826 | −5.412 | 59.643 | 1.00 | 99.59 | B |
| ATOM | 6603 | O | LYS | B | 375 | −22.551 | −6.318 | 58.856 | 1.00 | 101.85 | B |
| ATOM | 6604 | N | GLY | B | 376 | −22.650 | −5.527 | 60.956 | 1.00 | 75.73 | B |
| ATOM | 6605 | CA | GLY | B | 376 | −22.151 | −6.773 | 61.518 | 1.00 | 75.73 | B |
| ATOM | 6606 | C | GLY | B | 376 | −20.645 | −7.005 | 61.568 | 1.00 | 75.73 | B |
| ATOM | 6607 | O | GLY | B | 376 | −20.155 | −7.688 | 62.471 | 1.00 | 75.73 | B |
| ATOM | 6608 | N | GLU | B | 377 | −19.903 | −6.466 | 60.604 | 1.00 | 103.12 | B |
| ATOM | 6609 | CA | GLU | B | 377 | −18.454 | −6.639 | 60.592 | 1.00 | 103.12 | B |
| ATOM | 6610 | CB | GLU | B | 377 | −17.962 | −6.928 | 59.176 | 1.00 | 131.99 | B |
| ATOM | 6611 | CG | GLU | B | 377 | −16.511 | −7.369 | 59.114 | 1.00 | 131.99 | B |
| ATOM | 6612 | CD | GLU | B | 377 | −16.055 | −7.689 | 57.702 | 1.00 | 131.99 | B |
| ATOM | 6613 | OE1 | GLU | B | 377 | −14.933 | −8.220 | 57.544 | 1.00 | 131.99 | B |
| ATOM | 6614 | OE2 | GLU | B | 377 | −16.817 | −7.406 | 56.751 | 1.00 | 131.99 | B |
| ATOM | 6615 | C | GLU | B | 377 | −17.793 | −5.373 | 61.119 | 1.00 | 103.12 | B |
| ATOM | 6616 | O | GLU | B | 377 | −17.946 | −4.295 | 60.538 | 1.00 | 103.12 | B |
| ATOM | 6617 | N | TYR | B | 378 | −17.062 | −5.505 | 62.224 | 1.00 | 82.73 | B |
| ATOM | 6618 | CA | TYR | B | 378 | −16.398 | −4.355 | 62.831 | 1.00 | 82.73 | B |
| ATOM | 6619 | CB | TYR | B | 378 | −16.424 | −4.470 | 64.355 | 1.00 | 86.28 | B |
| ATOM | 6620 | CG | TYR | B | 378 | −17.816 | −4.300 | 64.912 | 1.00 | 86.28 | B |
| ATOM | 6621 | CD1 | TYR | B | 378 | −18.629 | −5.405 | 65.167 | 1.00 | 86.28 | B |
| ATOM | 6622 | CE1 | TYR | B | 378 | −19.952 | −5.242 | 65.587 | 1.00 | 86.28 | B |
| ATOM | 6623 | CD2 | TYR | B | 378 | −18.355 | −3.027 | 65.098 | 1.00 | 86.28 | B |
| ATOM | 6624 | CE2 | TYR | B | 378 | −19.675 | −2.853 | 65.516 | 1.00 | 86.28 | B |
| ATOM | 6625 | CZ | TYR | B | 378 | −20.467 | −3.962 | 65.755 | 1.00 | 86.28 | B |
| ATOM | 6626 | OH | TYR | B | 378 | −21.774 | −3.780 | 66.137 | 1.00 | 86.28 | B |
| ATOM | 6627 | C | TYR | B | 378 | −14.983 | −4.074 | 62.351 | 1.00 | 82.73 | B |
| ATOM | 6628 | O | TYR | B | 378 | −14.016 | −4.191 | 63.102 | 1.00 | 82.73 | B |
| ATOM | 6629 | N | LYS | B | 379 | −14.894 | −3.704 | 61.076 | 1.00 | 64.12 | B |
| ATOM | 6630 | CA | LYS | B | 379 | −13.649 | −3.335 | 60.421 | 1.00 | 64.12 | B |
| ATOM | 6631 | CB | LYS | B | 379 | −13.205 | −4.417 | 59.443 | 1.00 | 79.93 | B |
| ATOM | 6632 | CG | LYS | B | 379 | −12.772 | −5.708 | 60.090 | 1.00 | 79.93 | B |
| ATOM | 6633 | CD | LYS | B | 379 | −12.363 | −6.715 | 59.024 | 1.00 | 79.93 | B |
| ATOM | 6634 | CE | LYS | B | 379 | −12.115 | −8.105 | 59.606 | 1.00 | 79.93 | B |
| ATOM | 6635 | NZ | LYS | B | 379 | −11.891 | −9.128 | 58.530 | 1.00 | 79.93 | B |
| ATOM | 6636 | C | LYS | B | 379 | −13.948 | −2.044 | 59.659 | 1.00 | 64.12 | B |
| ATOM | 6637 | O | LYS | B | 379 | −15.009 | −1.910 | 59.044 | 1.00 | 64.12 | B |
| ATOM | 6638 | N | TYR | B | 380 | −13.029 | −1.088 | 59.723 | 1.00 | 56.89 | B |
| ATOM | 6639 | CA | TYR | B | 380 | −13.215 | 0.172 | 59.014 | 1.00 | 56.89 | B |
| ATOM | 6640 | CB | TYR | B | 380 | −12.456 | 1.282 | 59.729 | 1.00 | 55.69 | B |
| ATOM | 6641 | CG | TYR | B | 380 | −12.668 | 2.651 | 59.145 | 1.00 | 55.69 | B |
| ATOM | 6642 | CD1 | TYR | B | 380 | −13.741 | 3.437 | 59.547 | 1.00 | 55.69 | B |
| ATOM | 6643 | CE1 | TYR | B | 380 | −13.926 | 4.707 | 59.037 | 1.00 | 55.69 | B |
| ATOM | 6644 | CD2 | TYR | B | 380 | −11.780 | 3.169 | 58.202 | 1.00 | 55.69 | B |
| ATOM | 6645 | CE2 | TYR | B | 380 | −11.955 | 4.438 | 57.673 | 1.00 | 55.69 | B |
| ATOM | 6646 | CZ | TYR | B | 380 | −13.029 | 5.209 | 58.097 | 1.00 | 55.69 | B |
| ATOM | 6647 | OH | TYR | B | 380 | −13.192 | 6.491 | 57.605 | 1.00 | 55.69 | B |
| ATOM | 6648 | C | TYR | B | 380 | −12.647 | −0.018 | 57.607 | 1.00 | 56.89 | B |
| ATOM | 6649 | O | TYR | B | 380 | −11.441 | −0.189 | 57.446 | 1.00 | 56.89 | B |
| ATOM | 6650 | N | VAL | B | 381 | −13.510 | −0.003 | 56.596 | 1.00 | 55.30 | B |
| ATOM | 6651 | CA | VAL | B | 381 | −13.067 | −0.196 | 55.215 | 1.00 | 55.30 | B |
| ATOM | 6652 | CB | VAL | B | 381 | −14.216 | −0.745 | 54.342 | 1.00 | 43.51 | B |
| ATOM | 6653 | CG1 | VAL | B | 381 | −13.711 | −1.034 | 52.953 | 1.00 | 43.51 | B |
| ATOM | 6654 | CG2 | VAL | B | 381 | −14.771 | −2.008 | 54.959 | 1.00 | 43.51 | B |
| ATOM | 6655 | C | VAL | B | 381 | −12.550 | 1.113 | 54.622 | 1.00 | 55.30 | B |
| ATOM | 6656 | O | VAL | B | 381 | −13.289 | 2.094 | 54.533 | 1.00 | 55.30 | B |
| ATOM | 6657 | N | ASN | B | 382 | −11.291 | 1.110 | 54.195 | 1.00 | 62.53 | B |
| ATOM | 6658 | CA | ASN | B | 382 | −10.658 | 2.302 | 53.668 | 1.00 | 62.53 | B |
| ATOM | 6659 | CB | ASN | B | 382 | −9.443 | 1.943 | 52.836 | 1.00 | 40.47 | B |
| ATOM | 6660 | CG | ASN | B | 382 | −8.429 | 3.091 | 52.757 | 1.00 | 40.47 | B |
| ATOM | 6661 | OD1 | ASN | B | 382 | −8.789 | 4.283 | 52.791 | 1.00 | 40.47 | B |
| ATOM | 6662 | ND2 | ASN | B | 382 | −7.152 | 2.730 | 52.631 | 1.00 | 40.47 | B |
| ATOM | 6663 | C | ASN | B | 382 | −11.529 | 3.246 | 52.853 | 1.00 | 62.53 | B |
| ATOM | 6664 | O | ASN | B | 382 | −12.002 | 4.272 | 53.362 | 1.00 | 62.53 | B |
| ATOM | 6665 | N | GLY | B | 383 | −11.751 | 2.925 | 51.586 | 1.00 | 32.25 | B |
| ATOM | 6666 | CA | GLY | B | 383 | −12.546 | 3.849 | 50.788 | 1.00 | 32.25 | B |
| ATOM | 6667 | C | GLY | B | 383 | −14.038 | 3.976 | 51.059 | 1.00 | 32.25 | B |
| ATOM | 6668 | O | GLY | B | 383 | −14.720 | 4.712 | 50.354 | 1.00 | 32.25 | B |
| ATOM | 6669 | N | ARG | B | 384 | −14.557 | 3.278 | 52.063 | 1.00 | 53.48 | B |
| ATOM | 6670 | CA | ARG | B | 384 | −15.988 | 3.320 | 52.339 | 1.00 | 53.48 | B |
| ATOM | 6671 | CB | ARG | B | 384 | −16.384 | 2.120 | 53.194 | 1.00 | 52.95 | B |
| ATOM | 6672 | CG | ARG | B | 384 | −17.881 | 1.948 | 53.376 | 1.00 | 52.95 | B |
| ATOM | 6673 | CD | ARG | B | 384 | −18.183 | 0.549 | 53.887 | 1.00 | 52.95 | B |
| ATOM | 6674 | NE | ARG | B | 384 | −17.688 | −0.461 | 52.957 | 1.00 | 52.95 | B |
| ATOM | 6675 | CZ | ARG | B | 384 | −17.612 | −1.762 | 53.220 | 1.00 | 52.95 | B |
| ATOM | 6676 | NH1 | ARG | B | 384 | −17.998 | −2.246 | 54.394 | 1.00 | 52.95 | B |
| ATOM | 6677 | NH2 | ARG | B | 384 | −17.137 | −2.585 | 52.300 | 1.00 | 52.95 | B |
| ATOM | 6678 | C | ARG | B | 384 | −16.438 | 4.595 | 53.015 | 1.00 | 53.48 | B |
| ATOM | 6679 | O | ARG | B | 384 | −15.683 | 5.212 | 53.755 | 1.00 | 53.48 | B |
| ATOM | 6680 | N | TYR | B | 385 | −17.676 | 4.988 | 52.748 | 1.00 | 59.57 | B |
| ATOM | 6681 | CA | TYR | B | 385 | −18.237 | 6.186 | 53.351 | 1.00 | 59.57 | B |
| ATOM | 6682 | CB | TYR | B | 385 | −19.312 | 6.771 | 52.446 | 1.00 | 74.84 | B |
| ATOM | 6683 | CG | TYR | B | 385 | −20.065 | 7.891 | 53.103 | 1.00 | 74.84 | B |
| ATOM | 6684 | CD1 | TYR | B | 385 | −19.488 | 9.150 | 53.256 | 1.00 | 74.84 | B |
| ATOM | 6685 | CE1 | TYR | B | 385 | −20.145 | 10.163 | 53.941 | 1.00 | 74.84 | B |
| ATOM | 6686 | CD2 | TYR | B | 385 | −21.326 | 7.675 | 53.646 | 1.00 | 74.84 | B |
| ATOM | 6687 | CE2 | TYR | B | 385 | −21.993 | 8.680 | 54.335 | 1.00 | 74.84 | B |
| ATOM | 6688 | CZ | TYR | B | 385 | −21.396 | 9.920 | 54.481 | 1.00 | 74.84 | B |
| ATOM | 6689 | OH | TYR | B | 385 | −22.050 | 10.906 | 55.179 | 1.00 | 74.84 | B |
| ATOM | 6690 | C | TYR | B | 385 | −18.859 | 5.878 | 54.721 | 1.00 | 59.57 | B |
| ATOM | 6691 | O | TYR | B | 385 | −19.464 | 4.822 | 54.912 | 1.00 | 59.57 | B |
| ATOM | 6692 | N | TYR | B | 386 | −18.711 | 6.790 | 55.677 | 1.00 | 57.04 | B |
| ATOM | 6693 | CA | TYR | B | 386 | −19.294 | 6.586 | 57.001 | 1.00 | 57.04 | B |
| ATOM | 6694 | CB | TYR | B | 386 | −18.291 | 5.988 | 57.994 | 1.00 | 48.70 | B |
| ATOM | 6695 | CG | TYR | B | 386 | −17.765 | 4.610 | 57.710 | 1.00 | 48.70 | B |
| ATOM | 6696 | CD1 | TYR | B | 386 | −16.555 | 4.433 | 57.053 | 1.00 | 48.70 | B |
| ATOM | 6697 | CE1 | TYR | B | 386 | −16.033 | 3.161 | 56.824 | 1.00 | 48.70 | B |
| ATOM | 6698 | CD2 | TYR | B | 386 | −18.454 | 3.473 | 58.136 | 1.00 | 48.70 | B |
| ATOM | 6699 | CE2 | TYR | B | 386 | −17.945 | 2.189 | 57.915 | 1.00 | 48.70 | B |
| ATOM | 6700 | CZ | TYR | B | 386 | −16.732 | 2.043 | 57.257 | 1.00 | 48.70 | B |
| ATOM | 6701 | OH | TYR | B | 386 | −16.215 | 0.787 | 57.021 | 1.00 | 48.70 | B |
| ATOM | 6702 | C | TYR | B | 386 | −19.765 | 7.892 | 57.634 | 1.00 | 57.04 | B |
| ATOM | 6703 | O | TYR | B | 386 | −18.976 | 8.834 | 57.761 | 1.00 | 57.04 | B |
| ATOM | 6704 | N | PRO | B | 387 | −21.054 | 7.979 | 58.029 | 1.00 | 65.54 | B |
| ATOM | 6705 | CD | PRO | B | 387 | −22.176 | 7.078 | 57.716 | 1.00 | 123.15 | B |
| ATOM | 6706 | CA | PRO | B | 387 | −21.534 | 9.214 | 58.667 | 1.00 | 65.54 | B |
| ATOM | 6707 | CB | PRO | B | 387 | −23.014 | 8.933 | 58.872 | 1.00 | 123.15 | B |
| ATOM | 6708 | CG | PRO | B | 387 | −23.345 | 8.025 | 57.718 | 1.00 | 123.15 | B |
| ATOM | 6709 | C | PRO | B | 387 | −20.742 | 9.193 | 59.980 | 1.00 | 65.54 | B |
| ATOM | 6710 | O | PRO | B | 387 | −20.498 | 8.106 | 60.507 | 1.00 | 65.54 | B |
| ATOM | 6711 | N | GLU | B | 388 | −20.336 | 10.332 | 60.540 | 1.00 | 115.41 | B |
| ATOM | 6712 | CA | GLU | B | 388 | −19.512 | 10.193 | 61.735 | 1.00 | 115.41 | B |
| ATOM | 6713 | CB | GLU | B | 388 | −18.043 | 10.269 | 61.326 | 1.00 | 103.13 | B |
| ATOM | 6714 | CG | GLU | B | 388 | −17.600 | 11.635 | 60.860 | 1.00 | 103.13 | B |
| ATOM | 6715 | CD | GLU | B | 388 | −16.099 | 11.724 | 60.738 | 1.00 | 103.13 | B |
| ATOM | 6716 | OE1 | GLU | B | 388 | −15.411 | 11.478 | 61.753 | 1.00 | 103.13 | B |
| ATOM | 6717 | OE2 | GLU | B | 388 | −15.605 | 12.035 | 59.635 | 1.00 | 103.13 | B |
| ATOM | 6718 | C | GLU | B | 388 | −19.655 | 10.975 | 63.032 | 1.00 | 115.41 | B |
| ATOM | 6719 | O | GLU | B | 388 | −18.695 | 11.017 | 63.796 | 1.00 | 115.41 | B |
| ATOM | 6720 | N | TRP | B | 389 | −20.799 | 11.580 | 63.326 | 1.00 | 91.26 | B |
| ATOM | 6721 | CA | TRP | B | 389 | −20.903 | 12.290 | 64.607 | 1.00 | 91.26 | B |
| ATOM | 6722 | CB | TRP | B | 389 | −20.870 | 11.285 | 65.770 | 1.00 | 113.13 | B |
| ATOM | 6723 | CG | TRP | B | 389 | −22.195 | 10.873 | 66.332 | 1.00 | 113.13 | B |
| ATOM | 6724 | CD2 | TRP | B | 389 | −23.482 | 11.427 | 66.023 | 1.00 | 113.13 | B |
| ATOM | 6725 | CE2 | TRP | B | 389 | −24.428 | 10.745 | 66.817 | 1.00 | 113.13 | B |
| ATOM | 6726 | CE3 | TRP | B | 389 | −23.929 | 12.435 | 65.154 | 1.00 | 113.13 | B |
| ATOM | 6727 | CD1 | TRP | B | 389 | −22.411 | 9.907 | 67.271 | 1.00 | 113.13 | B |
| ATOM | 6728 | NE1 | TRP | B | 389 | −23.746 | 9.823 | 67.568 | 1.00 | 113.13 | B |
| ATOM | 6729 | CZ2 | TRP | B | 389 | −25.798 | 11.037 | 66.768 | 1.00 | 113.13 | B |
| ATOM | 6730 | CZ3 | TRP | B | 389 | −25.291 | 12.726 | 65.106 | 1.00 | 113.13 | B |
| ATOM | 6731 | CH2 | TRP | B | 389 | −26.208 | 12.028 | 65.910 | 1.00 | 113.13 | B |
| ATOM | 6732 | C | TRP | B | 389 | −19.755 | 13.282 | 64.816 | 1.00 | 91.26 | B |
| ATOM | 6733 | O | TRP | B | 389 | −18.872 | 13.044 | 65.645 | 1.00 | 91.26 | B |
| ATOM | 6734 | N | GLY | B | 390 | −19.767 | 14.391 | 64.083 | 1.00 | 80.15 | B |
| ATOM | 6735 | CA | GLY | B | 390 | −18.703 | 15.369 | 64.226 | 1.00 | 80.15 | B |
| ATOM | 6736 | C | GLY | B | 390 | −18.615 | 16.025 | 65.594 | 1.00 | 80.15 | B |
| ATOM | 6737 | O | GLY | B | 390 | −19.608 | 16.114 | 66.317 | 1.00 | 80.15 | B |
| ATOM | 6738 | N | ARG | B | 391 | −17.413 | 16.472 | 65.955 | 1.00 | 104.00 | B |
| ATOM | 6739 | CA | ARG | B | 391 | −17.184 | 17.153 | 67.228 | 1.00 | 104.00 | B |
| ATOM | 6740 | CB | ARG | B | 391 | −15.759 | 16.887 | 67.721 | 1.00 | 86.99 | B |
| ATOM | 6741 | CG | ARG | B | 391 | −15.436 | 15.407 | 67.917 | 1.00 | 86.99 | B |
| ATOM | 6742 | CD | ARG | B | 391 | −15.615 | 14.943 | 69.369 | 1.00 | 86.99 | B |
| ATOM | 6743 | NE | ARG | B | 391 | −14.386 | 15.065 | 70.169 | 1.00 | 86.99 | B |
| ATOM | 6744 | CZ | ARG | B | 391 | −13.911 | 16.203 | 70.679 | 1.00 | 86.99 | B |
| ATOM | 6745 | NH1 | ARG | B | 391 | −14.560 | 17.350 | 70.486 | 1.00 | 86.99 | B |
| ATOM | 6746 | NH2 | ARG | B | 391 | −12.780 | 16.195 | 71.379 | 1.00 | 86.99 | B |
| ATOM | 6747 | C | ARG | B | 391 | −17.382 | 18.645 | 66.954 | 1.00 | 104.00 | B |
| ATOM | 6748 | O | ARG | B | 391 | −17.457 | 19.059 | 65.799 | 1.00 | 104.00 | B |
| ATOM | 6749 | N | LYS | B | 392 | −17.481 | 19.456 | 68.000 | 1.00 | 69.99 | B |
| ATOM | 6750 | CA | LYS | B | 392 | −17.676 | 20.889 | 67.805 | 1.00 | 69.99 | B |
| ATOM | 6751 | CB | LYS | B | 392 | −18.893 | 21.379 | 68.599 | 1.00 | 106.30 | B |
| ATOM | 6752 | CG | LYS | B | 392 | −19.159 | 22.876 | 68.457 | 1.00 | 106.30 | B |
| ATOM | 6753 | CD | LYS | B | 392 | −20.211 | 23.359 | 69.445 | 1.00 | 106.30 | B |
| ATOM | 6754 | CE | LYS | B | 392 | −20.329 | 24.874 | 69.415 | 1.00 | 106.30 | B |
| ATOM | 6755 | NZ | LYS | B | 392 | −21.213 | 25.391 | 70.498 | 1.00 | 106.30 | B |
| ATOM | 6756 | C | LYS | B | 392 | −16.439 | 21.679 | 68.229 | 1.00 | 69.99 | B |
| ATOM | 6757 | O | LYS | B | 392 | −16.072 | 21.694 | 69.408 | 1.00 | 69.99 | B |
| ATOM | 6758 | N | SER | B | 393 | −15.798 | 22.336 | 67.265 | 1.00 | 52.64 | B |
| ATOM | 6759 | CA | SER | B | 393 | −14.598 | 23.128 | 67.530 | 1.00 | 52.64 | B |
| ATOM | 6760 | CB | SER | B | 393 | −13.384 | 22.208 | 67.726 | 1.00 | 70.94 | B |
| ATOM | 6761 | OG | SER | B | 393 | −12.184 | 22.944 | 67.901 | 1.00 | 70.94 | B |
| ATOM | 6762 | C | SER | B | 393 | −14.337 | 24.067 | 66.370 | 1.00 | 52.64 | B |
| ATOM | 6763 | O | SER | B | 393 | −14.818 | 23.833 | 65.264 | 1.00 | 52.64 | B |
| ATOM | 6764 | N | ASN | B | 394 | −13.590 | 25.139 | 66.616 | 1.00 | 53.04 | B |
| ATOM | 6765 | CA | ASN | B | 394 | −13.272 | 26.068 | 65.541 | 1.00 | 53.04 | B |
| ATOM | 6766 | CB | ASN | B | 394 | −12.779 | 27.388 | 66.100 | 1.00 | 59.87 | B |
| ATOM | 6767 | CG | ASN | B | 394 | −13.882 | 28.184 | 66.735 | 1.00 | 59.87 | B |
| ATOM | 6768 | OD1 | ASN | B | 394 | −14.950 | 28.366 | 66.143 | 1.00 | 59.87 | B |
| ATOM | 6769 | ND2 | ASN | B | 394 | −13.634 | 28.675 | 67.949 | 1.00 | 59.87 | B |
| ATOM | 6770 | C | ASN | B | 394 | −12.203 | 25.456 | 64.653 | 1.00 | 53.04 | B |
| ATOM | 6771 | O | ASN | B | 394 | −12.010 | 25.873 | 63.511 | 1.00 | 53.04 | B |
| ATOM | 6772 | N | TYR | B | 395 | −11.515 | 24.463 | 65.196 | 1.00 | 60.10 | B |
| ATOM | 6773 | CA | TYR | B | 395 | −10.477 | 23.767 | 64.475 | 1.00 | 60.10 | B |
| ATOM | 6774 | CB | TYR | B | 395 | −9.331 | 23.424 | 65.423 | 1.00 | 48.11 | B |
| ATOM | 6775 | CG | TYR | B | 395 | −8.622 | 24.617 | 65.997 | 1.00 | 48.11 | B |
| ATOM | 6776 | CD1 | TYR | B | 395 | −8.970 | 25.126 | 67.238 | 1.00 | 48.11 | B |
| ATOM | 6777 | CE1 | TYR | B | 395 | −8.312 | 26.234 | 67.774 | 1.00 | 48.11 | B |
| ATOM | 6778 | CD2 | TYR | B | 395 | −7.595 | 25.244 | 65.295 | 1.00 | 48.11 | B |
| ATOM | 6779 | CE2 | TYR | B | 395 | −6.934 | 26.349 | 65.817 | 1.00 | 48.11 | B |
| ATOM | 6780 | CZ | TYR | B | 395 | −7.299 | 26.839 | 67.059 | 1.00 | 48.11 | B |
| ATOM | 6781 | OH | TYR | B | 395 | −6.659 | 27.945 | 67.579 | 1.00 | 48.11 | B |
| ATOM | 6782 | C | TYR | B | 395 | −11.043 | 22.475 | 63.882 | 1.00 | 60.10 | B |
| ATOM | 6783 | O | TYR | B | 395 | −12.028 | 21.937 | 64.379 | 1.00 | 60.10 | B |
| ATOM | 6784 | N | GLU | B | 396 | −10.422 | 21.983 | 62.816 | 1.00 | 59.53 | B |
| ATOM | 6785 | CA | GLU | B | 396 | −10.831 | 20.731 | 62.187 | 1.00 | 59.53 | B |
| ATOM | 6786 | CB | GLU | B | 396 | −10.779 | 20.856 | 60.670 | 1.00 | 114.69 | B |
| ATOM | 6787 | CG | GLU | B | 396 | −11.438 | 22.095 | 60.121 | 1.00 | 114.69 | B |
| ATOM | 6788 | CD | GLU | B | 396 | −11.130 | 22.295 | 58.650 | 1.00 | 114.69 | B |
| ATOM | 6789 | OE1 | GLU | B | 396 | −9.931 | 22.311 | 58.292 | 1.00 | 114.69 | B |
| ATOM | 6790 | OE2 | GLU | B | 396 | −12.080 | 22.439 | 57.854 | 1.00 | 114.69 | B |
| ATOM | 6791 | C | GLU | B | 396 | −9.811 | 19.675 | 62.628 | 1.00 | 59.53 | B |
| ATOM | 6792 | O | GLU | B | 396 | −8.609 | 19.958 | 62.709 | 1.00 | 59.53 | B |
| ATOM | 6793 | N | PRO | B | 397 | −10.265 | 18.448 | 62.930 | 1.00 | 52.19 | B |
| ATOM | 6794 | CD | PRO | B | 397 | −11.608 | 17.867 | 62.818 | 1.00 | 49.76 | B |
| ATOM | 6795 | CA | PRO | B | 397 | −9.292 | 17.432 | 63.347 | 1.00 | 52.19 | B |
| ATOM | 6796 | CB | PRO | B | 397 | −10.131 | 16.154 | 63.424 | 1.00 | 49.76 | B |
| ATOM | 6797 | CG | PRO | B | 397 | −11.287 | 16.433 | 62.518 | 1.00 | 49.76 | B |
| ATOM | 6798 | C | PRO | B | 397 | −8.149 | 17.352 | 62.340 | 1.00 | 52.19 | B |
| ATOM | 6799 | O | PRO | B | 397 | −8.369 | 17.334 | 61.128 | 1.00 | 52.19 | B |
| ATOM | 6800 | N | GLY | B | 398 | −6.926 | 17.320 | 62.851 | 1.00 | 39.52 | B |
| ATOM | 6801 | CA | GLY | B | 398 | −5.772 | 17.287 | 61.975 | 1.00 | 39.52 | B |
| ATOM | 6802 | C | GLY | B | 398 | −4.944 | 18.532 | 62.247 | 1.00 | 39.52 | B |
| ATOM | 6803 | O | GLY | B | 398 | −3.718 | 18.465 | 62.277 | 1.00 | 39.52 | B |
| ATOM | 6804 | N | GLU | B | 399 | −5.617 | 19.667 | 62.447 | 1.00 | 44.99 | B |
| ATOM | 6805 | CA | GLU | B | 399 | −4.944 | 20.931 | 62.738 | 1.00 | 44.99 | B |
| ATOM | 6806 | CB | GLU | B | 399 | −5.921 | 22.106 | 62.632 | 1.00 | 61.78 | B |
| ATOM | 6807 | CG | GLU | B | 399 | −6.636 | 22.184 | 61.293 | 1.00 | 61.78 | B |
| ATOM | 6808 | CD | GLU | B | 399 | −7.567 | 23.382 | 61.162 | 1.00 | 61.78 | B |
| ATOM | 6809 | OE1 | GLU | B | 399 | −8.292 | 23.690 | 62.128 | 1.00 | 61.78 | B |
| ATOM | 6810 | OE2 | GLU | B | 399 | −7.588 | 24.008 | 60.081 | 1.00 | 61.78 | B |
| ATOM | 6811 | C | GLU | B | 399 | −4.445 | 20.819 | 64.162 | 1.00 | 44.99 | B |
| ATOM | 6812 | O | GLU | B | 399 | −5.163 | 20.327 | 65.022 | 1.00 | 44.99 | B |
| ATOM | 6813 | N | PRO | B | 400 | −3.206 | 21.258 | 64.441 | 1.00 | 59.04 | B |
| ATOM | 6814 | CD | PRO | B | 400 | −2.156 | 21.823 | 63.584 | 1.00 | 34.85 | B |
| ATOM | 6815 | CA | PRO | B | 400 | −2.741 | 21.135 | 65.824 | 1.00 | 54.12 | B |
| ATOM | 6816 | CB | PRO | B | 400 | −1.328 | 21.723 | 65.778 | 1.00 | 83.46 | B |
| ATOM | 6817 | CG | PRO | B | 400 | −1.338 | 22.583 | 64.576 | 1.00 | 34.85 | B |
| ATOM | 6818 | C | PRO | B | 400 | −3.644 | 21.804 | 66.847 | 1.00 | 44.87 | B |
| ATOM | 6819 | O | PRO | B | 400 | −3.603 | 21.470 | 68.029 | 1.00 | 45.08 | B |
| ATOM | 6820 | N | GLY | B | 401 | −4.485 | 22.723 | 66.393 | 1.00 | 55.79 | B |
| ATOM | 6821 | CA | GLY | B | 401 | −5.385 | 23.398 | 67.313 | 1.00 | 55.35 | B |
| ATOM | 6822 | C | GLY | B | 401 | −6.571 | 22.581 | 67.812 | 1.00 | 55.14 | B |
| ATOM | 6823 | O | GLY | B | 401 | −7.279 | 23.011 | 68.717 | 1.00 | 55.85 | B |
| ATOM | 6824 | N | PHE | B | 402 | −6.804 | 21.408 | 67.235 | 1.00 | 66.03 | B |
| ATOM | 6825 | CA | PHE | B | 402 | −7.928 | 20.583 | 67.662 | 1.00 | 66.03 | B |
| ATOM | 6826 | CB | PHE | B | 402 | −8.132 | 19.423 | 66.706 | 1.00 | 159.04 | B |
| ATOM | 6827 | CG | PHE | B | 402 | −9.500 | 18.853 | 66.773 | 1.00 | 57.37 | B |
| ATOM | 6828 | CD1 | PHE | B | 402 | −10.568 | 19.532 | 66.207 | 1.00 | 57.37 | B |
| ATOM | 6829 | CD2 | PHE | B | 402 | −9.744 | 17.669 | 67.454 | 1.00 | 57.37 | B |
| ATOM | 6830 | CE1 | PHE | B | 402 | −11.867 | 19.041 | 66.321 | 1.00 | 57.37 | B |
| ATOM | 6831 | CE2 | PHE | B | 402 | −11.035 | 17.171 | 67.576 | 1.00 | 57.37 | B |
| ATOM | 6832 | CZ | PHE | B | 402 | −12.100 | 17.861 | 67.008 | 1.00 | 57.37 | B |
| ATOM | 6833 | C | PHE | B | 402 | −7.691 | 20.039 | 69.067 | 1.00 | 66.03 | B |
| ATOM | 6834 | O | PHE | B | 402 | −6.634 | 19.469 | 69.343 | 1.00 | 66.03 | B |
| ATOM | 6835 | N | PRO | B | 403 | −8.682 | 20.197 | 69.971 | 1.00 | 65.57 | B |
| ATOM | 6836 | CD | PRO | B | 403 | −9.953 | 20.872 | 69.689 | 1.00 | 39.09 | B |
| ATOM | 6837 | CA | PRO | B | 403 | −8.650 | 19.754 | 71.374 | 1.00 | 65.57 | B |
| ATOM | 6838 | CB | PRO | B | 403 | −9.902 | 20.386 | 71.980 | 1.00 | 143.92 | B |
| ATOM | 6839 | CG | PRO | B | 403 | −10.275 | 21.451 | 71.034 | 1.00 | 39.09 | B |
| ATOM | 6840 | C | PRO | B | 403 | −8.677 | 18.250 | 71.552 | 1.00 | 65.57 | B |
| ATOM | 6841 | O | PRO | B | 403 | −9.727 | 17.635 | 71.433 | 1.00 | 65.57 | B |
| ATOM | 6842 | N | TRP | B | 404 | −7.525 | 17.652 | 71.815 | 1.00 | 72.55 | B |
| ATOM | 6843 | CA | TRP | B | 404 | −7.462 | 16.214 | 72.052 | 1.00 | 72.55 | B |
| ATOM | 6844 | CB | TRP | B | 404 | −6.518 | 15.509 | 71.068 | 1.00 | 75.70 | B |
| ATOM | 6845 | CG | TRP | B | 404 | −7.053 | 15.330 | 69.671 | 1.00 | 68.94 | B |
| ATOM | 6846 | CD2 | TRP | B | 404 | −8.210 | 14.575 | 69.273 | 1.00 | 68.94 | B |
| ATOM | 6847 | CE2 | TRP | B | 404 | −8.293 | 14.653 | 67.866 | 1.00 | 68.94 | B |
| ATOM | 6848 | CE3 | TRP | B | 404 | −9.183 | 13.840 | 69.968 | 1.00 | 68.94 | B |
| ATOM | 6849 | CD1 | TRP | B | 404 | −6.505 | 15.818 | 68.522 | 1.00 | 68.94 | B |
| ATOM | 6850 | NE1 | TRP | B | 404 | −7.242 | 15.417 | 67.436 | 1.00 | 68.94 | B |
| ATOM | 6851 | CZ2 | TRP | B | 404 | −9.309 | 14.026 | 67.137 | 1.00 | 68.94 | B |
| ATOM | 6852 | CZ3 | TRP | B | 404 | −10.196 | 13.213 | 69.242 | 1.00 | 68.94 | B |
| ATOM | 6853 | CH2 | TRP | B | 404 | −10.248 | 13.312 | 67.840 | 1.00 | 68.94 | B |
| ATOM | 6854 | C | TRP | B | 404 | −6.910 | 16.081 | 73.458 | 1.00 | 72.55 | B |
| ATOM | 6855 | O | TRP | B | 404 | −5.904 | 16.702 | 73.787 | 1.00 | 72.55 | B |
| ATOM | 6856 | N | ASN | B | 405 | −7.564 | 15.286 | 74.293 | 1.00 | 63.60 | B |
| ATOM | 6857 | CA | ASN | B | 405 | −7.097 | 15.114 | 75.657 | 1.00 | 63.63 | B |
| ATOM | 6858 | CB | ASN | B | 405 | −8.227 | 14.598 | 76.535 | 1.00 | 76.15 | B |
| ATOM | 6859 | CG | ASN | B | 405 | −7.996 | 14.891 | 77.986 | 1.00 | 76.15 | B |
| ATOM | 6860 | OD1 | ASN | B | 405 | −7.133 | 14.293 | 78.622 | 1.00 | 76.15 | B |
| ATOM | 6861 | ND2 | ASN | B | 405 | −8.758 | 15.836 | 78.523 | 1.00 | 76.15 | B |
| ATOM | 6862 | C | ASN | B | 405 | −5.917 | 14.149 | 75.693 | 1.00 | 72.57 | B |
| ATOM | 6863 | O | ASN | B | 405 | −6.050 | 12.983 | 76.092 | 1.00 | 65.63 | B |
| ATOM | 6864 | N | ILE | B | 406 | −4.763 | 14.647 | 75.259 | 1.00 | 69.69 | B |
| ATOM | 6865 | CA | ILE | B | 406 | −3.550 | 13.850 | 75.232 | 1.00 | 61.89 | B |
| ATOM | 6866 | CB | ILE | B | 406 | −2.875 | 13.909 | 73.851 | 1.00 | 64.56 | B |
| ATOM | 6867 | CG2 | ILE | B | 406 | −1.672 | 12.977 | 73.812 | 1.00 | 52.18 | B |
| ATOM | 6868 | CG1 | ILE | B | 406 | −3.880 | 13.517 | 72.770 | 1.00 | 52.18 | B |
| ATOM | 6869 | CD1 | ILE | B | 406 | −3.296 | 13.494 | 71.364 | 1.00 | 52.18 | B |
| ATOM | 6870 | C | ILE | B | 406 | −2.572 | 14.384 | 76.259 | 1.00 | 68.76 | B |
| ATOM | 6871 | O | ILE | B | 406 | −2.467 | 15.588 | 76.457 | 1.00 | 63.86 | B |
| ATOM | 6872 | N | LYS | B | 407 | −1.868 | 13.480 | 76.921 | 1.00 | 46.85 | B |
| ATOM | 6873 | CA | LYS | B | 407 | −0.867 | 13.851 | 77.913 | 1.00 | 42.45 | B |
| ATOM | 6874 | CB | LYS | B | 407 | −1.407 | 13.700 | 79.336 | 1.00 | 107.76 | B |
| ATOM | 6875 | CG | LYS | B | 407 | −2.577 | 14.601 | 79.675 | 1.00 | 93.86 | B |
| ATOM | 6876 | CD | LYS | B | 407 | −2.895 | 14.524 | 81.162 | 1.00 | 93.86 | B |
| ATOM | 6877 | CE | LYS | B | 407 | −4.161 | 15.295 | 81.518 | 1.00 | 93.86 | B |
| ATOM | 6878 | NZ | LYS | B | 407 | −5.373 | 14.685 | 80.906 | 1.00 | 93.86 | B |
| ATOM | 6879 | C | LYS | B | 407 | 0.270 | 12.877 | 77.718 | 1.00 | 51.76 | B |
| ATOM | 6880 | O | LYS | B | 407 | 0.033 | 11.698 | 77.447 | 1.00 | 50.70 | B |
| ATOM | 6881 | N | LYS | B | 408 | 1.503 | 13.358 | 77.824 | 1.00 | 55.97 | B |
| ATOM | 6882 | CA | LYS | B | 408 | 2.638 | 12.463 | 77.670 | 1.00 | 55.56 | B |
| ATOM | 6883 | CB | LYS | B | 408 | 3.927 | 13.250 | 77.485 | 1.00 | 60.30 | B |
| ATOM | 6884 | CG | LYS | B | 408 | 5.115 | 12.372 | 77.158 | 1.00 | 59.10 | B |
| ATOM | 6885 | CD | LYS | B | 408 | 6.295 | 13.206 | 76.729 | 1.00 | 59.10 | B |
| ATOM | 6886 | CE | LYS | B | 408 | 7.456 | 12.336 | 76.328 | 1.00 | 59.10 | B |
| ATOM | 6887 | NZ | LYS | B | 408 | 8.574 | 13.154 | 75.770 | 1.00 | 59.10 | B |
| ATOM | 6888 | C | LYS | B | 408 | 2.677 | 11.686 | 78.967 | 1.00 | 55.69 | B |
| ATOM | 6889 | O | LYS | B | 408 | 2.992 | 12.239 | 80.011 | 1.00 | 54.26 | B |
| ATOM | 6890 | N | ASP | B | 409 | 2.337 | 10.408 | 78.927 | 1.00 | 73.13 | B |
| ATOM | 6891 | CA | ASP | B | 409 | 2.331 | 9.660 | 80.169 | 1.00 | 73.31 | B |
| ATOM | 6892 | CB | ASP | B | 409 | 1.688 | 8.271 | 79.977 | 1.00 | 95.09 | B |
| ATOM | 6893 | CG | ASP | B | 409 | 2.204 | 7.534 | 78.762 | 1.00 | 87.91 | B |
| ATOM | 6894 | OD1 | ASP | B | 409 | 3.435 | 7.484 | 78.569 | 1.00 | 87.91 | B |
| ATOM | 6895 | OD2 | ASP | B | 409 | 1.371 | 6.986 | 78.011 | 1.00 | 87.91 | B |
| ATOM | 6896 | C | ASP | B | 409 | 3.700 | 9.551 | 80.844 | 1.00 | 73.22 | B |
| ATOM | 6897 | O | ASP | B | 409 | 4.733 | 9.804 | 80.222 | 1.00 | 76.95 | B |
| ATOM | 6898 | N | LYS | B | 410 | 3.661 | 9.205 | 82.134 | 1.00 | 94.50 | B |
| ATOM | 6899 | CA | LYS | B | 410 | 4.825 | 9.037 | 83.008 | 1.00 | 100.94 | B |
| ATOM | 6900 | CB | LYS | B | 410 | 4.566 | 7.901 | 84.008 | 1.00 | 173.56 | B |
| ATOM | 6901 | CG | LYS | B | 410 | 4.493 | 6.492 | 83.393 | 1.00 | 113.01 | B |
| ATOM | 6902 | CD | LYS | B | 410 | 3.214 | 6.259 | 82.585 | 1.00 | 113.01 | B |
| ATOM | 6903 | CE | LYS | B | 410 | 3.199 | 4.873 | 81.946 | 1.00 | 113.01 | B |
| ATOM | 6904 | NZ | LYS | B | 410 | 1.969 | 4.633 | 81.140 | 1.00 | 113.01 | B |
| ATOM | 6905 | C | LYS | B | 410 | 6.133 | 8.762 | 82.287 | 1.00 | 99.76 | B |
| ATOM | 6906 | O | LYS | B | 410 | 6.139 | 8.230 | 81.183 | 1.00 | 99.29 | B |
| ATOM | 6907 | N | ASP | B | 411 | 7.238 | 9.109 | 82.941 | 1.00 | 75.44 | B |
| ATOM | 6908 | CA | ASP | B | 411 | 8.586 | 8.927 | 82.393 | 1.00 | 72.95 | B |
| ATOM | 6909 | CB | ASP | B | 411 | 9.569 | 8.538 | 83.502 | 1.00 | 179.79 | B |
| ATOM | 6910 | CG | ASP | B | 411 | 10.186 | 9.746 | 84.186 | 1.00 | 179.79 | B |
| ATOM | 6911 | OD1 | ASP | B | 411 | 10.714 | 10.628 | 83.475 | 1.00 | 179.79 | B |
| ATOM | 6912 | OD2 | ASP | B | 411 | 10.152 | 9.812 | 85.431 | 1.00 | 179.79 | B |
| ATOM | 6913 | C | ASP | B | 411 | 8.704 | 7.925 | 81.253 | 1.00 | 74.56 | B |
| ATOM | 6914 | O | ASP | B | 411 | 9.055 | 6.762 | 81.465 | 1.00 | 78.74 | B |
| ATOM | 6915 | N | ALA | B | 412 | 8.421 | 8.400 | 80.041 | 1.00 | 98.93 | B |
| ATOM | 6916 | CA | ALA | B | 412 | 8.473 | 7.580 | 78.837 | 1.00 | 111.95 | B |
| ATOM | 6917 | CB | ALA | B | 412 | 7.375 | 6.520 | 78.873 | 1.00 | 77.71 | B |
| ATOM | 6918 | C | ALA | B | 412 | 8.302 | 8.460 | 77.605 | 1.00 | 106.65 | B |
| ATOM | 6919 | O | ALA | B | 412 | 8.002 | 9.648 | 77.715 | 1.00 | 101.70 | B |
| ATOM | 6920 | N | ASN | B | 413 | 8.485 | 7.866 | 76.432 | 1.00 | 63.52 | B |
| ATOM | 6921 | CA | ASN | B | 413 | 8.356 | 8.598 | 75.179 | 1.00 | 56.33 | B |
| ATOM | 6922 | CB | ASN | B | 413 | 9.305 | 8.032 | 74.138 | 1.00 | 62.96 | B |
| ATOM | 6923 | CG | ASN | B | 413 | 10.746 | 8.149 | 74.554 | 1.00 | 62.96 | B |
| ATOM | 6924 | OD1 | ASN | B | 413 | 11.163 | 9.179 | 75.084 | 1.00 | 62.96 | B |
| ATOM | 6925 | ND2 | ASN | B | 413 | 11.525 | 7.099 | 74.302 | 1.00 | 62.96 | B |
| ATOM | 6926 | C | ASN | B | 413 | 6.955 | 8.521 | 74.630 | 1.00 | 60.91 | B |
| ATOM | 6927 | O | ASN | B | 413 | 6.594 | 9.259 | 73.724 | 1.00 | 60.60 | B |
| ATOM | 6928 | N | THR | B | 414 | 6.169 | 7.627 | 75.204 | 1.00 | 40.68 | B |
| ATOM | 6929 | CA | THR | B | 414 | 4.805 | 7.396 | 74.772 | 1.00 | 43.72 | B |
| ATOM | 6930 | CB | THR | B | 414 | 4.413 | 5.967 | 75.147 | 1.00 | 108.47 | B |
| ATOM | 6931 | OG1 | THR | B | 414 | 3.081 | 5.725 | 74.720 | 1.00 | 78.96 | B |
| ATOM | 6932 | CG2 | THR | B | 414 | 4.516 | 5.750 | 76.640 | 1.00 | 78.96 | B |
| ATOM | 6933 | C | THR | B | 414 | 3.762 | 8.410 | 75.281 | 1.00 | 43.18 | B |
| ATOM | 6934 | O | THR | B | 414 | 4.042 | 9.192 | 76.199 | 1.00 | 44.25 | B |
| ATOM | 6935 | N | TYR | B | 415 | 2.581 | 8.417 | 74.656 | 1.00 | 48.38 | B |
| ATOM | 6936 | CA | TYR | B | 415 | 1.491 | 9.328 | 75.027 | 1.00 | 49.41 | B |
| ATOM | 6937 | CB | TYR | B | 415 | 1.243 | 10.359 | 73.930 | 1.00 | 77.23 | B |
| ATOM | 6938 | CG | TYR | B | 415 | 2.373 | 11.322 | 73.717 | 1.00 | 44.17 | B |
| ATOM | 6939 | CD1 | TYR | B | 415 | 3.572 | 10.897 | 73.160 | 1.00 | 44.17 | B |
| ATOM | 6940 | CE1 | TYR | B | 415 | 4.623 | 11.791 | 72.962 | 1.00 | 44.17 | B |
| ATOM | 6941 | CD2 | TYR | B | 415 | 2.245 | 12.673 | 74.078 | 1.00 | 44.17 | B |
| ATOM | 6942 | CE2 | TYR | B | 415 | 3.287 | 13.575 | 73.887 | 1.00 | 44.17 | B |
| ATOM | 6943 | CZ | TYR | B | 415 | 4.473 | 13.125 | 73.328 | 1.00 | 44.17 | B |
| ATOM | 6944 | OH | TYR | B | 415 | 5.520 | 13.992 | 73.128 | 1.00 | 44.17 | B |
| ATOM | 6945 | C | TYR | B | 415 | 0.179 | 8.589 | 75.260 | 1.00 | 56.97 | B |
| ATOM | 6946 | O | TYR | B | 415 | 0.009 | 7.451 | 74.812 | 1.00 | 51.62 | B |
| ATOM | 6947 | N | SER | B | 416 | −0.756 | 9.248 | 75.943 | 1.00 | 71.09 | B |
| ATOM | 6948 | CA | SER | B | 416 | −2.060 | 8.654 | 76.215 | 1.00 | 69.04 | B |
| ATOM | 6949 | CB | SER | B | 416 | −2.153 | 8.203 | 77.668 | 1.00 | 94.22 | B |
| ATOM | 6950 | OG | SER | B | 416 | −1.393 | 7.024 | 77.870 | 1.00 | 70.60 | B |
| ATOM | 6951 | C | SER | B | 416 | −3.172 | 9.633 | 75.906 | 1.00 | 75.23 | B |
| ATOM | 6952 | O | SER | B | 416 | −2.991 | 10.845 | 76.011 | 1.00 | 73.60 | B |
| ATOM | 6953 | N | LEU | B | 417 | −4.327 | 9.095 | 75.530 | 1.00 | 56.22 | B |
| ATOM | 6954 | CA | LEU | B | 417 | −5.477 | 9.914 | 75.164 | 1.00 | 55.12 | B |
| ATOM | 6955 | CB | LEU | B | 417 | −5.605 | 9.936 | 73.644 | 1.00 | 89.62 | B |
| ATOM | 6956 | CG | LEU | B | 417 | −7.015 | 10.181 | 73.112 | 1.00 | 47.73 | B |
| ATOM | 6957 | CD1 | LEU | B | 417 | −7.353 | 11.660 | 73.198 | 1.00 | 47.73 | B |
| ATOM | 6958 | CD2 | LEU | B | 417 | −7.102 | 9.702 | 71.682 | 1.00 | 47.73 | B |
| ATOM | 6959 | C | LEU | B | 417 | −6.796 | 9.431 | 75.767 | 1.00 | 66.63 | B |
| ATOM | 6960 | O | LEU | B | 417 | −6.982 | 8.237 | 76.019 | 1.00 | 61.73 | B |
| ATOM | 6961 | N | THR | B | 418 | −7.710 | 10.374 | 75.977 | 1.00 | 62.97 | B |
| ATOM | 6962 | CA | THR | B | 418 | −9.027 | 10.074 | 76.521 | 1.00 | 56.03 | B |
| ATOM | 6963 | CB | THR | B | 418 | −9.115 | 10.420 | 78.016 | 1.00 | 107.83 | B |
| ATOM | 6964 | OG1 | THR | B | 418 | −8.344 | 9.475 | 78.769 | 1.00 | 76.24 | B |
| ATOM | 6965 | CG2 | THR | B | 418 | −10.562 | 10.389 | 78.486 | 1.00 | 76.24 | B |
| ATOM | 6966 | C | THR | B | 418 | −10.063 | 10.897 | 75.782 | 1.00 | 71.22 | B |
| ATOM | 6967 | O | THR | B | 418 | −10.072 | 12.118 | 75.889 | 1.00 | 64.69 | B |
| ATOM | 6968 | N | ASP | B | 419 | −10.936 | 10.238 | 75.029 | 1.00 | 63.11 | B |
| ATOM | 6969 | CA | ASP | B | 419 | −11.959 | 10.966 | 74.289 | 1.00 | 68.22 | B |
| ATOM | 6970 | CB | ASP | B | 419 | −11.832 | 10.677 | 72.794 | 1.00 | 96.97 | B |
| ATOM | 6971 | CG | ASP | B | 419 | −12.611 | 11.658 | 71.955 | 1.00 | 74.56 | B |
| ATOM | 6972 | OD1 | ASP | B | 419 | −12.465 | 12.875 | 72.188 | 1.00 | 74.56 | B |
| ATOM | 6973 | OD2 | ASP | B | 419 | −13.368 | 11.221 | 71.064 | 1.00 | 74.56 | B |
| ATOM | 6974 | C | ASP | B | 419 | −13.353 | 10.601 | 74.791 | 1.00 | 72.31 | B |
| ATOM | 6975 | O | ASP | B | 419 | −13.726 | 9.429 | 74.814 | 1.00 | 67.20 | B |
| ATOM | 6976 | N | LEU | B | 420 | −14.118 | 11.613 | 75.189 | 1.00 | 89.62 | B |
| ATOM | 6977 | CA | LEU | B | 420 | −15.460 | 11.407 | 75.723 | 1.00 | 94.31 | B |
| ATOM | 6978 | CB | LEU | B | 420 | −15.620 | 12.242 | 76.996 | 1.00 | 149.06 | B |
| ATOM | 6979 | CG | LEU | B | 420 | −16.715 | 11.837 | 77.979 | 1.00 | 108.01 | B |
| ATOM | 6980 | CD1 | LEU | B | 420 | −16.391 | 10.464 | 78.552 | 1.00 | 108.01 | B |
| ATOM | 6981 | CD2 | LEU | B | 420 | −16.818 | 12.870 | 79.093 | 1.00 | 108.01 | B |
| ATOM | 6982 | C | LEU | B | 420 | −16.545 | 11.787 | 74.715 | 1.00 | 96.88 | B |
| ATOM | 6983 | O | LEU | B | 420 | −16.315 | 12.613 | 73.837 | 1.00 | 96.20 | B |
| ATOM | 6984 | N | VAL | B | 421 | −17.726 | 11.183 | 74.840 | 1.00 | 84.12 | B |
| ATOM | 6985 | CA | VAL | B | 421 | −18.843 | 11.480 | 73.940 | 1.00 | 82.63 | B |
| ATOM | 6986 | CB | VAL | B | 421 | −18.890 | 10.485 | 72.776 | 1.00 | 110.16 | B |
| ATOM | 6987 | CG1 | VAL | B | 421 | −20.124 | 10.715 | 71.929 | 1.00 | 65.59 | B |
| ATOM | 6988 | CG2 | VAL | B | 421 | −17.671 | 10.659 | 71.932 | 1.00 | 65.59 | B |
| ATOM | 6989 | C | VAL | B | 421 | −20.193 | 11.458 | 74.644 | 1.00 | 84.89 | B |
| ATOM | 6990 | O | VAL | B | 421 | −20.481 | 10.549 | 75.422 | 1.00 | 80.49 | B |
| ATOM | 6991 | N | ARG | B | 422 | −21.019 | 12.459 | 74.356 | 1.00 | 105.63 | B |
| ATOM | 6992 | CA | ARG | B | 422 | −22.349 | 12.577 | 74.949 | 1.00 | 107.76 | B |
| ATOM | 6993 | CB | ARG | B | 422 | −22.808 | 14.038 | 74.874 | 1.00 | 189.06 | B |
| ATOM | 6994 | CG | ARG | B | 422 | −23.213 | 14.663 | 76.198 | 1.00 | 149.65 | B |
| ATOM | 6995 | CD | ARG | B | 422 | −24.602 | 14.228 | 76.632 | 1.00 | 149.65 | B |
| ATOM | 6996 | NE | ARG | B | 422 | −25.032 | 14.949 | 77.826 | 1.00 | 149.65 | B |
| ATOM | 6997 | CZ | ARG | B | 422 | −26.255 | 14.885 | 78.343 | 1.00 | 149.65 | B |
| ATOM | 6998 | NH1 | ARG | B | 422 | −27.181 | 14.126 | 77.770 | 1.00 | 149.65 | B |
| ATOM | 6999 | NH2 | ARG | B | 422 | −26.552 | 15.587 | 79.430 | 1.00 | 149.65 | B |
| ATOM | 7000 | C | ARG | B | 422 | −23.336 | 11.686 | 74.189 | 1.00 | 104.99 | B |
| ATOM | 7001 | O | ARG | B | 422 | −23.808 | 12.061 | 73.119 | 1.00 | 95.66 | B |
| ATOM | 7002 | N | THR | B | 423 | −23.648 | 10.513 | 74.733 | 1.00 | 76.06 | B |
| ATOM | 7003 | CA | THR | B | 423 | −24.577 | 9.594 | 74.079 | 1.00 | 85.71 | B |
| ATOM | 7004 | CB | THR | B | 423 | −24.548 | 8.196 | 74.743 | 1.00 | 131.51 | B |
| ATOM | 7005 | OG1 | THR | B | 423 | −23.210 | 7.693 | 74.730 | 1.00 | 87.30 | B |
| ATOM | 7006 | CG2 | THR | B | 423 | −25.440 | 7.215 | 73.989 | 1.00 | 87.30 | B |
| ATOM | 7007 | C | THR | B | 423 | −26.018 | 10.099 | 74.100 | 1.00 | 83.93 | B |
| ATOM | 7008 | O | THR | B | 423 | −26.881 | 9.474 | 74.699 | 1.00 | 82.42 | B |
| ATOM | 7009 | N | THR | B | 424 | −26.283 | 11.225 | 73.446 | 1.00 | 132.80 | B |
| ATOM | 7010 | CA | THR | B | 424 | −27.639 | 11.763 | 73.408 | 1.00 | 132.80 | B |
| ATOM | 7011 | CB | THR | B | 424 | −27.689 | 13.115 | 72.674 | 1.00 | 198.02 | B |
| ATOM | 7012 | OG1 | THR | B | 424 | −26.801 | 14.036 | 73.318 | 1.00 | 143.83 | B |
| ATOM | 7013 | CG2 | THR | B | 424 | −29.102 | 13.688 | 72.699 | 1.00 | 143.83 | B |
| ATOM | 7014 | C | THR | B | 424 | −28.539 | 10.767 | 72.688 | 1.00 | 132.80 | B |
| ATOM | 7015 | O | THR | B | 424 | −28.064 | 9.955 | 71.893 | 1.00 | 132.80 | B |
| ATOM | 7016 | N | SER | B | 425 | −29.836 | 10.831 | 72.969 | 1.00 | 133.08 | B |
| ATOM | 7017 | CA | SER | B | 425 | −30.797 | 9.918 | 72.361 | 1.00 | 133.08 | B |
| ATOM | 7018 | CB | SER | B | 425 | −32.103 | 9.954 | 73.146 | 1.00 | 173.00 | B |
| ATOM | 7019 | OG | SER | B | 425 | −33.015 | 9.017 | 72.621 | 1.00 | 68.94 | B |
| ATOM | 7020 | C | SER | B | 425 | −31.083 | 10.195 | 70.885 | 1.00 | 133.08 | B |
| ATOM | 7021 | O | SER | B | 425 | −31.854 | 9.475 | 70.250 | 1.00 | 133.08 | B |
| ATOM | 7022 | N | LYS | B | 426 | −30.458 | 11.233 | 70.342 | 1.00 | 157.81 | B |
| ATOM | 7023 | CA | LYS | B | 426 | −30.648 | 11.593 | 68.941 | 1.00 | 157.81 | B |
| ATOM | 7024 | CB | LYS | B | 426 | −29.774 | 12.795 | 68.583 | 1.00 | 200.84 | B |
| ATOM | 7025 | CG | LYS | B | 426 | −29.916 | 13.977 | 69.517 | 1.00 | 153.66 | B |
| ATOM | 7026 | CD | LYS | B | 426 | −28.880 | 15.036 | 69.195 | 1.00 | 153.66 | B |
| ATOM | 7027 | CE | LYS | B | 426 | −28.911 | 16.160 | 70.210 | 1.00 | 153.66 | B |
| ATOM | 7028 | NZ | LYS | B | 426 | −27.865 | 17.176 | 69.915 | 1.00 | 153.66 | B |
| ATOM | 7029 | C | LYS | B | 426 | −30.284 | 10.430 | 68.024 | 1.00 | 157.81 | B |
| ATOM | 7030 | O | LYS | B | 426 | −30.951 | 10.187 | 67.029 | 1.00 | 157.81 | B |
| ATOM | 7031 | N | MET | B | 427 | −29.218 | 9.716 | 68.371 | 1.00 | 119.58 | B |
| ATOM | 7032 | CA | MET | B | 427 | −28.740 | 8.590 | 67.575 | 1.00 | 119.58 | B |
| ATOM | 7033 | CB | MET | B | 427 | −27.386 | 8.114 | 68.106 | 1.00 | 121.41 | B |
| ATOM | 7034 | CG | MET | B | 427 | −27.369 | 7.836 | 69.599 | 1.00 | 121.41 | B |
| ATOM | 7035 | SD | MET | B | 427 | −25.743 | 7.338 | 70.179 | 1.00 | 121.41 | B |
| ATOM | 7036 | CE | MET | B | 427 | −24.896 | 8.924 | 70.232 | 1.00 | 121.41 | B |
| ATOM | 7037 | C | MET | B | 427 | −29.710 | 7.420 | 67.537 | 1.00 | 119.58 | B |
| ATOM | 7038 | O | MET | B | 427 | −30.894 | 7.576 | 67.834 | 1.00 | 119.58 | B |
| ATOM | 7039 | N | SER | B | 428 | −29.197 | 6.247 | 67.168 | 1.00 | 108.22 | B |
| ATOM | 7040 | CA | SER | B | 428 | −30.019 | 5.049 | 67.083 | 1.00 | 109.87 | B |
| ATOM | 7041 | CB | SER | B | 428 | −31.022 | 5.185 | 65.932 | 1.00 | 107.01 | B |
| ATOM | 7042 | OG | SER | B | 428 | −30.361 | 5.289 | 64.680 | 1.00 | 103.72 | B |
| ATOM | 7043 | C | SER | B | 428 | −29.200 | 3.778 | 66.883 | 1.00 | 105.82 | B |
| ATOM | 7044 | O | SER | B | 428 | −29.677 | 2.834 | 66.262 | 1.00 | 105.70 | B |
| ATOM | 7045 | N | SER | B | 429 | −27.976 | 3.748 | 67.404 | 1.00 | 136.30 | B |
| ATOM | 7046 | CA | SER | B | 429 | −27.126 | 2.566 | 67.261 | 1.00 | 134.97 | B |
| ATOM | 7047 | CB | SER | B | 429 | −26.509 | 2.512 | 65.860 | 1.00 | 124.74 | B |
| ATOM | 7048 | OG | SER | B | 429 | −27.497 | 2.378 | 64.857 | 1.00 | 124.74 | B |
| ATOM | 7049 | C | SER | B | 429 | −25.997 | 2.516 | 68.282 | 1.00 | 133.35 | B |
| ATOM | 7050 | O | SER | B | 429 | −25.301 | 1.507 | 68.393 | 1.00 | 122.05 | B |
| ATOM | 7051 | N | GLN | B | 430 | −25.819 | 3.600 | 69.028 | 1.00 | 110.66 | B |
| ATOM | 7052 | CA | GLN | B | 430 | −24.745 | 3.681 | 70.012 | 1.00 | 110.66 | B |
| ATOM | 7053 | CB | GLN | B | 430 | −24.795 | 2.520 | 70.998 | 1.00 | 194.45 | B |
| ATOM | 7054 | CG | GLN | B | 430 | −23.718 | 2.631 | 72.053 | 1.00 | 123.44 | B |
| ATOM | 7055 | CD | GLN | B | 430 | −23.712 | 1.469 | 73.008 | 1.00 | 123.44 | B |
| ATOM | 7056 | OE1 | GLN | B | 430 | −23.371 | 0.342 | 72.636 | 1.00 | 123.44 | B |
| ATOM | 7057 | NE2 | GLN | B | 430 | −24.097 | 1.731 | 74.253 | 1.00 | 123.44 | B |
| ATOM | 7058 | C | GLN | B | 430 | −23.424 | 3.639 | 69.256 | 1.00 | 110.66 | B |
| ATOM | 7059 | O | GLN | B | 430 | −23.051 | 2.607 | 68.687 | 1.00 | 110.66 | B |
| ATOM | 7060 | N | PRO | B | 431 | −22.682 | 4.757 | 69.266 | 1.00 | 78.77 | B |
| ATOM | 7061 | CD | PRO | B | 431 | −22.818 | 5.929 | 70.148 | 1.00 | 62.22 | B |
| ATOM | 7062 | CA | PRO | B | 431 | −21.411 | 4.808 | 68.556 | 1.00 | 78.76 | B |
| ATOM | 7063 | CB | PRO | B | 431 | −20.946 | 6.234 | 68.800 | 1.00 | 161.18 | B |
| ATOM | 7064 | CG | PRO | B | 431 | −21.411 | 6.477 | 70.178 | 1.00 | 131.74 | B |
| ATOM | 7065 | C | PRO | B | 431 | −20.398 | 3.790 | 69.013 | 1.00 | 75.38 | B |
| ATOM | 7066 | O | PRO | B | 431 | −20.468 | 3.265 | 70.121 | 1.00 | 75.26 | B |
| ATOM | 7067 | N | VAL | B | 432 | −19.468 | 3.511 | 68.112 | 1.00 | 68.48 | B |
| ATOM | 7068 | CA | VAL | B | 432 | −18.375 | 2.597 | 68.355 | 1.00 | 70.86 | B |
| ATOM | 7069 | CB | VAL | B | 432 | −18.395 | 1.455 | 67.345 | 1.00 | 91.60 | B |
| ATOM | 7070 | CG1 | VAL | B | 432 | −18.260 | 2.000 | 65.949 | 1.00 | 63.68 | B |
| ATOM | 7071 | CG2 | VAL | B | 432 | −17.295 | 0.498 | 67.643 | 1.00 | 63.68 | B |
| ATOM | 7072 | C | VAL | B | 432 | −17.164 | 3.504 | 68.134 | 1.00 | 68.86 | B |
| ATOM | 7073 | O | VAL | B | 432 | −17.237 | 4.458 | 67.362 | 1.00 | 59.22 | B |
| ATOM | 7074 | N | CYS | B | 433 | −16.064 | 3.240 | 68.821 | 1.00 | 66.16 | B |
| ATOM | 7075 | CA | CYS | B | 433 | −14.894 | 4.094 | 68.676 | 1.00 | 62.04 | B |
| ATOM | 7076 | C | CYS | B | 433 | −13.911 | 3.573 | 67.634 | 1.00 | 59.68 | B |
| ATOM | 7077 | O | CYS | B | 433 | −13.441 | 2.435 | 67.729 | 1.00 | 60.42 | B |
| ATOM | 7078 | CB | CYS | B | 433 | −14.189 | 4.237 | 70.029 | 1.00 | 151.95 | B |
| ATOM | 7079 | SG | CYS | B | 433 | −12.789 | 5.401 | 70.030 | 1.00 | 99.06 | B |
| ATOM | 7080 | N | VAL | B | 434 | −13.610 | 4.401 | 66.629 | 1.00 | 64.71 | B |
| ATOM | 7081 | CA | VAL | B | 434 | −12.644 | 4.030 | 65.590 | 1.00 | 60.70 | B |
| ATOM | 7082 | CB | VAL | B | 434 | −13.149 | 4.349 | 64.188 | 1.00 | 65.15 | B |
| ATOM | 7083 | CG1 | VAL | B | 434 | −12.205 | 3.752 | 63.163 | 1.00 | 46.93 | B |
| ATOM | 7084 | CG2 | VAL | B | 434 | −14.529 | 3.795 | 64.000 | 1.00 | 46.93 | B |
| ATOM | 7085 | C | VAL | B | 434 | −11.378 | 4.840 | 65.831 | 1.00 | 71.10 | B |
| ATOM | 7086 | O | VAL | B | 434 | −11.404 | 6.071 | 65.768 | 1.00 | 62.16 | B |
| ATOM | 7087 | N | VAL | B | 435 | −10.278 | 4.142 | 66.101 | 1.00 | 50.72 | B |
| ATOM | 7088 | CA | VAL | B | 435 | −9.005 | 4.786 | 66.399 | 1.00 | 42.46 | B |
| ATOM | 7089 | CB | VAL | B | 435 | −8.341 | 4.103 | 67.616 | 1.00 | 53.98 | B |
| ATOM | 7090 | CG1 | VAL | B | 435 | −6.917 | 4.591 | 67.803 | 1.00 | 33.31 | B |
| ATOM | 7091 | CG2 | VAL | B | 435 | −9.160 | 4.384 | 68.850 | 1.00 | 33.31 | B |
| ATOM | 7092 | C | VAL | B | 435 | −8.017 | 4.790 | 65.238 | 1.00 | 50.38 | B |
| ATOM | 7093 | O | VAL | B | 435 | −7.650 | 3.729 | 64.724 | 1.00 | 50.97 | B |
| ATOM | 7094 | N | PHE | B | 436 | −7.581 | 5.981 | 64.830 | 1.00 | 49.71 | B |
| ATOM | 7095 | CA | PHE | B | 436 | −6.613 | 6.108 | 63.746 | 1.00 | 45.91 | B |
| ATOM | 7096 | CB | PHE | B | 436 | −7.041 | 7.190 | 62.764 | 1.00 | 64.46 | B |
| ATOM | 7097 | CG | PHE | B | 436 | −8.322 | 6.889 | 62.061 | 1.00 | 39.23 | B |
| ATOM | 7098 | CD1 | PHE | B | 436 | −9.536 | 7.256 | 62.619 | 1.00 | 39.23 | B |
| ATOM | 7099 | CD2 | PEE | B | 436 | −8.318 | 6.228 | 60.834 | 1.00 | 39.23 | B |
| ATOM | 7100 | CE1 | PHE | B | 436 | −10.739 | 6.974 | 61.963 | 1.00 | 39.23 | B |
| ATOM | 7101 | CE2 | PHE | B | 436 | −9.511 | 5.937 | 60.168 | 1.00 | 39.23 | B |
| ATOM | 7102 | CZ | PHE | B | 436 | −10.723 | 6.310 | 60.732 | 1.00 | 39.23 | B |
| ATOM | 7103 | C | PHE | B | 436 | −5.232 | 6.439 | 64.298 | 1.00 | 53.74 | B |
| ATOM | 7104 | O | PHE | B | 436 | −5.039 | 7.480 | 64.921 | 1.00 | 53.12 | B |
| ATOM | 7105 | N | HIS | B | 437 | −4.281 | 5.537 | 64.074 | 1.00 | 37.45 | B |
| ATOM | 7106 | CA | HIS | B | 437 | −2.918 | 5.727 | 64.543 | 1.00 | 38.52 | B |
| ATOM | 7107 | CB | HIS | B | 437 | −2.772 | 5.186 | 65.949 | 1.00 | 66.86 | B |
| ATOM | 7108 | CG | HIS | B | 437 | −1.403 | 5.371 | 66.519 | 1.00 | 43.23 | B |
| ATOM | 7109 | CD2 | HIS | B | 437 | −0.354 | 4.520 | 66.607 | 1.00 | 43.23 | B |
| ATOM | 7110 | ND1 | HIS | B | 437 | −0.974 | 6.566 | 67.065 | 1.00 | 43.23 | B |
| ATOM | 7111 | CE1 | HIS | B | 437 | 0.277 | 6.441 | 67.464 | 1.00 | 43.23 | B |
| ATOM | 7112 | NE2 | HIS | B | 437 | 0.677 | 5.211 | 67.196 | 1.00 | 43.23 | B |
| ATOM | 7113 | C | HIS | B | 437 | −1.958 | 4.992 | 63.614 | 1.00 | 42.83 | B |
| ATOM | 7114 | O | HIS | B | 437 | −2.374 | 4.078 | 62.900 | 1.00 | 45.05 | B |
| ATOM | 7115 | N | ASP | B | 438 | −0.683 | 5.385 | 63.614 | 1.00 | 34.51 | B |
| ATOM | 7116 | CA | ASP | B | 438 | 0.315 | 4.755 | 62.746 | 1.00 | 36.42 | B |
| ATOM | 7117 | CB | ASP | B | 438 | 1.700 | 5.364 | 62.998 | 1.00 | 101.96 | B |
| ATOM | 7118 | CG | ASP | B | 438 | 1.932 | 6.637 | 62.189 | 1.00 | 72.92 | B |
| ATOM | 7119 | OD1 | ASP | B | 438 | 0.928 | 7.278 | 61.797 | 1.00 | 72.92 | B |
| ATOM | 7120 | OD2 | ASP | B | 438 | 3.114 | 6.995 | 61.953 | 1.00 | 72.92 | B |
| ATOM | 7121 | C | ASP | B | 438 | 0.340 | 3.247 | 62.918 | 1.00 | 37.76 | B |
| ATOM | 7122 | O | ASP | B | 438 | 0.360 | 2.511 | 61.938 | 1.00 | 41.32 | B |
| ATOM | 7123 | N | THR | B | 439 | 0.356 | 2.783 | 64.160 | 1.00 | 76.30 | B |
| ATOM | 7124 | CA | THR | B | 439 | 0.312 | 1.352 | 64.420 | 1.00 | 73.65 | B |
| ATOM | 7125 | CB | THR | B | 439 | 0.783 | 1.048 | 65.845 | 1.00 | 62.50 | B |
| ATOM | 7126 | OG1 | THR | B | 439 | −0.081 | 1.694 | 66.784 | 1.00 | 48.31 | B |
| ATOM | 7127 | CG2 | THR | B | 439 | 2.186 | 1.587 | 66.058 | 1.00 | 48.31 | B |
| ATOM | 7128 | C | THR | B | 439 | −1.189 | 1.127 | 64.272 | 1.00 | 79.42 | B |
| ATOM | 7129 | O | THR | B | 439 | −1.966 | 2.016 | 64.598 | 1.00 | 79.76 | B |
| ATOM | 7130 | N | LEU | B | 440 | −1.614 | −0.026 | 63.775 | 1.00 | 79.65 | B |
| ATOM | 7131 | CA | LEU | B | 440 | −3.047 | −0.272 | 63.549 | 1.00 | 81.67 | B |
| ATOM | 7132 | CB | LEU | B | 440 | −3.834 | −0.462 | 64.877 | 1.00 | 85.42 | B |
| ATOM | 7133 | CG | LEU | B | 440 | −4.141 | 0.414 | 66.125 | 1.00 | 65.42 | B |
| ATOM | 7134 | CD1 | LEU | B | 440 | −2.891 | 0.624 | 66.918 | 1.00 | 65.42 | B |
| ATOM | 7135 | CD2 | LEU | B | 440 | −4.793 | 1.733 | 65.775 | 1.00 | 65.42 | B |
| ATOM | 7136 | C | LEU | B | 440 | −3.708 | 0.831 | 62.706 | 1.00 | 91.62 | B |
| ATOM | 7137 | O | LEU | B | 440 | −4.429 | 1.664 | 63.239 | 1.00 | 89.84 | B |
| ATOM | 7138 | N | GLU | B | 441 | −3.469 | 0.820 | 61.391 | 1.00 | 62.37 | B |
| ATOM | 7139 | CA | GLU | B | 441 | −4.023 | 1.823 | 60.474 | 1.00 | 62.37 | B |
| ATOM | 7140 | CB | GLU | B | 441 | −4.139 | 1.242 | 59.064 | 1.00 | 200.84 | B |
| ATOM | 7141 | CG | GLU | B | 441 | −2.799 | 0.908 | 58.434 | 1.00 | 140.84 | B |
| ATOM | 7142 | CD | GLU | B | 441 | −2.923 | 0.493 | 56.979 | 1.00 | 140.84 | B |
| ATOM | 7143 | OE1 | GLU | B | 441 | −3.651 | −0.483 | 56.702 | 1.00 | 140.84 | B |
| ATOM | 7144 | OE2 | GLU | B | 441 | −2.292 | 1.140 | 56.113 | 1.00 | 140.84 | B |
| ATOM | 7145 | C | GLU | B | 441 | −5.373 | 2.407 | 60.901 | 1.00 | 62.37 | B |
| ATOM | 7146 | O | GLU | B | 441 | −5.584 | 3.624 | 60.844 | 1.00 | 62.37 | B |
| ATOM | 7147 | N | ALA | B | 442 | −6.288 | 1.544 | 61.329 | 1.00 | 71.33 | B |
| ATOM | 7148 | CA | ALA | B | 442 | −7.595 | 1.999 | 61.779 | 1.00 | 69.55 | B |
| ATOM | 7149 | CB | ALA | B | 442 | −8.371 | 2.578 | 60.608 | 1.00 | 33.99 | B |
| ATOM | 7150 | C | ALA | B | 442 | −8.363 | 0.846 | 62.396 | 1.00 | 65.54 | B |
| ATOM | 7151 | O | ALA | B | 442 | −8.819 | −0.031 | 61.678 | 1.00 | 62.95 | B |
| ATOM | 7152 | N | GLN | B | 443 | −8.500 | 0.842 | 63.723 | 1.00 | 61.28 | B |
| ATOM | 7153 | CA | GLN | B | 443 | −9.222 | −0.227 | 64.413 | 1.00 | 64.56 | B |
| ATOM | 7154 | CB | GLN | B | 443 | −8.339 | −0.881 | 65.472 | 1.00 | 90.32 | B |
| ATOM | 7155 | CG | GLN | B | 443 | −7.534 | −2.055 | 64.964 | 1.00 | 73.55 | B |
| ATOM | 7156 | CD | GLN | B | 443 | −7.067 | −2.953 | 66.100 | 1.00 | 73.55 | B |
| ATOM | 7157 | OE1 | GLN | B | 443 | −7.863 | −3.361 | 66.952 | 1.00 | 73.55 | B |
| ATOM | 7158 | NE2 | GLN | B | 443 | −5.776 | −3.269 | 66.117 | 1.00 | 73.55 | B |
| ATOM | 7159 | C | GLN | B | 443 | −10.509 | 0.240 | 65.068 | 1.00 | 66.73 | B |
| ATOM | 7160 | O | GLN | B | 443 | −10.626 | 1.392 | 65.500 | 1.00 | 67.79 | B |
| ATOM | 7161 | N | VAL | B | 444 | −11.476 | −0.667 | 65.138 | 1.00 | 60.59 | B |
| ATOM | 7162 | CA | VAL | B | 444 | −12.761 | −0.355 | 65.748 | 1.00 | 57.74 | B |
| ATOM | 7163 | CB | VAL | B | 444 | −13.908 | −0.880 | 64.882 | 1.00 | 74.38 | B |
| ATOM | 7164 | CG1 | VAL | B | 444 | −15.219 | −0.225 | 65.293 | 1.00 | 66.68 | B |
| ATOM | 7165 | CG2 | VAL | B | 444 | −13.604 | −0.603 | 63.425 | 1.00 | 66.68 | B |
| ATOM | 7166 | C | VAL | B | 444 | −12.847 | −0.971 | 67.151 | 1.00 | 61.56 | B |
| ATOM | 7167 | O | VAL | B | 444 | −12.361 | −2.085 | 67.376 | 1.00 | 63.82 | B |
| ATOM | 7168 | N | TYR | B | 445 | −13.441 | −0.232 | 68.091 | 1.00 | 61.03 | B |
| ATOM | 7169 | CA | TYR | B | 445 | −13.579 | −0.689 | 69.474 | 1.00 | 60.10 | B |
| ATOM | 7170 | CB | TYR | B | 445 | −12.553 | 0.008 | 70.358 | 1.00 | 82.74 | B |
| ATOM | 7171 | CG | TYR | B | 445 | −11.134 | −0.245 | 69.922 | 1.00 | 55.16 | B |
| ATOM | 7172 | CD1 | TYR | B | 445 | −10.489 | 0.609 | 69.020 | 1.00 | 55.16 | B |
| ATOM | 7173 | CE1 | TYR | B | 445 | −9.169 | 0.360 | 68.602 | 1.00 | 55.16 | B |
| ATOM | 7174 | CD2 | TYR | B | 445 | −10.432 | −1.352 | 70.395 | 1.00 | 55.16 | B |
| ATOM | 7175 | CE2 | TYR | B | 445 | −9.115 | −1.611 | 69.985 | 1.00 | 55.16 | B |
| ATOM | 7176 | CZ | TYR | B | 445 | −8.491 | −0.754 | 69.093 | 1.00 | 55.16 | B |
| ATOM | 7177 | OH | TYR | B | 445 | −7.193 | −1.014 | 68.717 | 1.00 | 55.16 | B |
| ATOM | 7178 | C | TYR | B | 445 | −14.985 | −0.410 | 69.982 | 1.00 | 61.56 | B |
| ATOM | 7179 | O | TYR | B | 445 | −15.533 | 0.678 | 69.761 | 1.00 | 64.42 | B |
| ATOM | 7180 | N | THR | B | 446 | −15.557 | −1.378 | 70.696 | 1.00 | 62.55 | B |
| ATOM | 7181 | CA | THR | B | 446 | −16.929 | −1.237 | 71.171 | 1.00 | 69.53 | B |
| ATOM | 7182 | CB | THR | B | 446 | −17.781 | −2.358 | 70.579 | 1.00 | 74.77 | B |
| ATOM | 7183 | OG1 | THR | B | 446 | −17.183 | −3.615 | 70.912 | 1.00 | 62.92 | B |
| ATOM | 7184 | CG2 | THR | B | 446 | −17.853 | −2.244 | 69.084 | 1.00 | 62.92 | B |
| ATOM | 7185 | C | THR | B | 446 | −17.193 | −1.222 | 72.679 | 1.00 | 79.33 | B |
| ATOM | 7186 | O | THR | B | 446 | −16.329 | −0.882 | 73.487 | 1.00 | 83.00 | B |
| ATOM | 7187 | N | CYS | B | 447 | −18.436 | −1.592 | 72.999 | 1.00 | 90.79 | B |
| ATOM | 7188 | CA | CYS | B | 447 | −19.040 | −1.724 | 74.334 | 1.00 | 91.77 | B |
| ATOM | 7189 | C | CYS | B | 447 | −19.220 | −0.516 | 75.242 | 1.00 | 91.80 | B |
| ATOM | 7190 | O | CYS | B | 447 | −18.380 | 0.371 | 75.316 | 1.00 | 88.66 | B |
| ATOM | 7191 | CB | CYS | B | 447 | −18.376 | −2.866 | 75.124 | 1.00 | 136.24 | B |
| ATOM | 7192 | SG | CYS | B | 447 | −16.599 | −2.717 | 75.446 | 1.00 | 121.77 | B |
| ATOM | 7193 | N | SER | B | 448 | −20.363 | −0.512 | 75.923 | 1.00 | 189.30 | B |
| ATOM | 7194 | CA | SER | B | 448 | −20.743 | 0.542 | 76.853 | 1.00 | 189.30 | B |
| ATOM | 7195 | CB | SER | B | 448 | −21.930 | 1.334 | 76.305 | 1.00 | 200.84 | B |
| ATOM | 7196 | OG | SER | B | 448 | −21.611 | 1.920 | 75.056 | 1.00 | 200.84 | B |
| ATOM | 7197 | C | SER | B | 448 | −21.114 | −0.086 | 78.191 | 1.00 | 189.30 | B |
| ATOM | 7198 | O | SER | B | 448 | −20.654 | −1.185 | 78.508 | 1.00 | 189.30 | B |
| ATOM | 7199 | N | GLU | B | 449 | −21.964 | 0.594 | 78.961 | 1.00 | 152.92 | B |
| ATOM | 7200 | CA | GLU | B | 449 | −22.357 | 0.110 | 80.283 | 1.00 | 152.92 | B |
| ATOM | 7201 | CB | GLU | B | 449 | −22.828 | −1.349 | 80.220 | 1.00 | 134.25 | B |
| ATOM | 7202 | CG | GLU | B | 449 | −24.262 | −1.530 | 79.753 | 1.00 | 134.25 | B |
| ATOM | 7203 | CD | GLU | B | 449 | −24.577 | −0.707 | 78.525 | 1.00 | 134.25 | B |
| ATOM | 7204 | OE1 | GLU | B | 449 | −23.842 | −0.838 | 77.524 | 1.00 | 134.25 | B |
| ATOM | 7205 | OE2 | GLU | B | 449 | −25.556 | 0.070 | 78.560 | 1.00 | 134.25 | B |
| ATOM | 7206 | C | GLU | B | 449 | −21.114 | 0.207 | 81.154 | 1.00 | 152.92 | B |
| ATOM | 7207 | O | GLU | B | 449 | −21.165 | 0.644 | 82.301 | 1.00 | 152.92 | B |
| ATOM | 7208 | N | GLY | B | 450 | −19.993 | −0.196 | 80.569 | 1.00 | 81.26 | B |
| ATOM | 7209 | CA | GLY | B | 450 | −18.711 | −0.176 | 81.241 | 1.00 | 81.26 | B |
| ATOM | 7210 | C | GLY | B | 450 | −17.949 | −1.353 | 80.681 | 1.00 | 81.26 | B |
| ATOM | 7211 | O | GLY | B | 450 | −16.900 | −1.740 | 81.198 | 1.00 | 81.26 | B |
| ATOM | 7212 | N | CYS | B | 451 | −18.491 | −1.917 | 79.605 | 1.00 | 167.43 | B |
| ATOM | 7213 | CA | CYS | B | 451 | −17.901 | −3.075 | 78.953 | 1.00 | 167.43 | B |
| ATOM | 7214 | CB | CYS | B | 451 | −16.505 | −2.758 | 78.451 | 1.00 | 161.96 | B |
| ATOM | 7215 | SG | CYS | B | 451 | −16.519 | −1.558 | 77.101 | 1.00 | 161.96 | B |
| ATOM | 7216 | C | CYS | B | 451 | −17.824 | −4.216 | 79.938 | 1.00 | 167.43 | B |
| ATOM | 7217 | O | CYS | B | 451 | −16.703 | −4.728 | 80.143 | 1.00 | 167.43 | B |
| ATOM | 7218 | OXT | CYS | B | 451 | −18.886 | −4.574 | 80.488 | 1.00 | 134.33 | B |
| ATOM | 7219 | CB | ARG | C | 1 | −15.415 | 15.094 | 12.139 | 1.00 | 133.76 | C |
| ATOM | 7220 | CG | ARG | C | 1 | −13.880 | 15.204 | 12.120 | 1.00 | 133.76 | C |
| ATOM | 7221 | CD | ARG | C | 1 | −13.343 | 16.332 | 11.234 | 1.00 | 133.76 | C |
| ATOM | 7222 | NE | ARG | C | 1 | −13.137 | 17.588 | 11.962 | 1.00 | 133.76 | C |
| ATOM | 7223 | CZ | ARG | C | 1 | −12.253 | 17.761 | 12.944 | 1.00 | 133.76 | C |
| ATOM | 7224 | NH1 | ARG | C | 1 | −11.476 | 16.759 | 13.333 | 1.00 | 133.76 | C |
| ATOM | 7225 | NH2 | ARG | C | 1 | −12.147 | 18.942 | 13.542 | 1.00 | 133.76 | C |
| ATOM | 7226 | C | ARG | C | 1 | −15.665 | 16.702 | 14.064 | 1.00 | 200.84 | C |
| ATOM | 7227 | O | ARG | C | 1 | −14.566 | 17.237 | 14.205 | 1.00 | 200.84 | C |
| ATOM | 7228 | N | ARG | C | 1 | −16.048 | 17.452 | 11.715 | 1.00 | 200.84 | C |
| ATOM | 7229 | CA | ARG | C | 1 | −16.178 | 16.317 | 12.672 | 1.00 | 200.84 | C |
| ATOM | 7230 | N | THR | C | 2 | −16.468 | 16.432 | 15.090 | 1.00 | 200.07 | C |
| ATOM | 7231 | CA | THR | C | 2 | −16.096 | 16.756 | 16.467 | 1.00 | 200.07 | C |
| ATOM | 7232 | CB | THR | C | 2 | −17.335 | 16.750 | 17.401 | 1.00 | 133.35 | C |
| ATOM | 7233 | OG1 | THR | C | 2 | −18.325 | 17.655 | 16.900 | 1.00 | 132.09 | C |
| ATOM | 7234 | CG2 | THR | C | 2 | −16.943 | 17.164 | 18.813 | 1.00 | 135.24 | C |
| ATOM | 7235 | C | THR | C | 2 | −15.101 | 15.748 | 17.031 | 1.00 | 200.07 | C |
| ATOM | 7236 | O | THR | C | 2 | −15.464 | 14.595 | 17.265 | 1.00 | 200.07 | C |
| ATOM | 7237 | N | PRO | C | 3 | −13.833 | 16.154 | 17.247 | 1.00 | 195.99 | C |
| ATOM | 7238 | CD | PRO | C | 3 | −13.112 | 17.401 | 16.935 | 1.00 | 82.77 | C |
| ATOM | 7239 | CA | PRO | C | 3 | −12.922 | 15.150 | 17.800 | 1.00 | 195.99 | C |
| ATOM | 7240 | CB | PRO | C | 3 | −11.708 | 15.981 | 18.213 | 1.00 | 81.01 | C |
| ATOM | 7241 | CG | PRO | C | 3 | −11.642 | 16.983 | 17.103 | 1.00 | 80.88 | C |
| ATOM | 7242 | C | PRO | C | 3 | −13.623 | 14.462 | 18.972 | 1.00 | 195.99 | C |
| ATOM | 7243 | O | PRO | C | 3 | −13.444 | 13.265 | 19.192 | 1.00 | 195.99 | C |
| ATOM | 7244 | N | SER | C | 4 | −14.440 | 15.233 | 19.696 | 1.00 | 127.11 | C |
| ATOM | 7245 | CA | SER | C | 4 | −15.226 | 14.731 | 20.825 | 1.00 | 123.68 | C |
| ATOM | 7246 | CB | SER | C | 4 | −14.460 | 13.658 | 21.611 | 1.00 | 119.49 | C |
| ATOM | 7247 | OG | SER | C | 4 | −13.558 | 14.236 | 22.535 | 1.00 | 121.44 | C |
| ATOM | 7248 | C | SER | C | 4 | −15.678 | 15.808 | 21.809 | 1.00 | 124.02 | C |
| ATOM | 7249 | O | SER | C | 4 | −16.748 | 15.678 | 22.402 | 1.00 | 124.17 | C |
| ATOM | 7250 | N | ASP | C | 5 | −14.867 | 16.856 | 21.975 | 1.00 | 61.06 | C |
| ATOM | 7251 | CA | ASP | C | 5 | −15.139 | 17.957 | 22.918 | 1.00 | 61.17 | C |
| ATOM | 7252 | CB | ASP | C | 5 | −16.604 | 17.918 | 23.376 | 1.00 | 145.50 | C |
| ATOM | 7253 | CG | ASP | C | 5 | −16.934 | 18.987 | 24.389 | 1.00 | 145.50 | C |
| ATOM | 7254 | OD1 | ASP | C | 5 | −18.045 | 18.929 | 24.956 | 1.00 | 145.50 | C |
| ATOM | 7255 | OD2 | ASP | C | 5 | −16.096 | 19.883 | 24.615 | 1.00 | 144.06 | C |
| ATOM | 7256 | C | ASP | C | 5 | −14.199 | 17.850 | 24.146 | 1.00 | 58.67 | C |
| ATOM | 7257 | O | ASP | C | 5 | −13.945 | 18.831 | 24.845 | 1.00 | 62.18 | C |
| ATOM | 7258 | N | LYS | C | 6 | −13.684 | 16.645 | 24.387 | 1.00 | 64.12 | C |
| ATOM | 7259 | CA | LYS | C | 6 | −12.773 | 16.357 | 25.498 | 1.00 | 60.50 | C |
| ATOM | 7260 | CB | LYS | C | 6 | −12.629 | 14.852 | 25.712 | 1.00 | 53.66 | C |
| ATOM | 7261 | CG | LYS | C | 6 | −13.806 | 14.094 | 26.271 | 1.00 | 60.07 | C |
| ATOM | 7262 | CD | LYS | C | 6 | −13.489 | 12.591 | 26.211 | 1.00 | 63.68 | C |
| ATOM | 7263 | CE | LYS | C | 6 | −14.520 | 11.759 | 26.948 | 1.00 | 66.95 | C |
| ATOM | 7264 | NZ | LYS | C | 6 | −14.544 | 12.094 | 28.400 | 1.00 | 68.14 | C |
| ATOM | 7265 | C | LYS | C | 6 | −11.362 | 16.849 | 25.234 | 1.00 | 55.83 | C |
| ATOM | 7266 | O | LYS | C | 6 | −10.818 | 16.621 | 24.153 | 1.00 | 59.71 | C |
| ATOM | 7267 | N | PRO | C | 7 | −10.741 | 17.530 | 26.207 | 1.00 | 53.63 | C |
| ATOM | 7268 | CD | PRO | C | 7 | −11.241 | 18.146 | 27.441 | 1.00 | 34.57 | C |
| ATOM | 7269 | CA | PRO | C | 7 | −9.371 | 17.969 | 25.929 | 1.00 | 51.67 | C |
| ATOM | 7270 | CB | PRO | C | 7 | −8.978 | 18.749 | 27.190 | 1.00 | 36.57 | C |
| ATOM | 7271 | CG | PRO | C | 7 | −9.961 | 18.319 | 28.218 | 1.00 | 35.44 | C |
| ATOM | 7272 | C | PRO | C | 7 | −8.541 | 16.706 | 25.730 | 1.00 | 49.73 | C |
| ATOM | 7273 | O | PRO | C | 7 | −8.666 | 15.748 | 26.488 | 1.00 | 50.74 | C |
| ATOM | 7274 | N | VAL | C | 8 | −7.712 | 16.696 | 24.699 | 1.00 | 28.08 | C |
| ATOM | 7275 | CA | VAL | C | 8 | −6.909 | 15.525 | 24.403 | 1.00 | 29.24 | C |
| ATOM | 7276 | CB | VAL | C | 8 | −7.725 | 14.526 | 23.504 | 1.00 | 30.42 | C |
| ATOM | 7277 | CG1 | VAL | C | 8 | −8.163 | 15.224 | 22.219 | 1.00 | 31.00 | C |
| ATOM | 7278 | CG2 | VAL | C | 8 | −6.900 | 13.300 | 23.161 | 1.00 | 33.12 | C |
| ATOM | 7279 | C | VAL | C | 8 | −5.637 | 15.922 | 23.663 | 1.00 | 31.74 | C |
| ATOM | 7280 | O | VAL | C | 8 | −5.541 | 17.011 | 23.097 | 1.00 | 30.98 | C |
| ATOM | 7281 | N | ALA | C | 9 | −4.660 | 15.026 | 23.661 | 1.00 | 38.53 | C |
| ATOM | 7282 | CA | ALA | C | 9 | −3.434 | 15.293 | 22.943 | 1.00 | 44.77 | C |
| ATOM | 7283 | CB | ALA | C | 9 | −2.642 | 16.376 | 23.640 | 1.00 | 46.11 | C |
| ATOM | 7284 | C | ALA | C | 9 | −2.602 | 14.041 | 22.828 | 1.00 | 45.75 | C |
| ATOM | 7285 | O | ALA | C | 9 | −2.485 | 13.266 | 23.781 | 1.00 | 43.73 | C |
| ATOM | 7286 | N | HIS | C | 10 | −2.051 | 13.836 | 21.641 | 1.00 | 39.78 | C |
| ATOM | 7287 | CA | HIS | C | 10 | −1.166 | 12.707 | 21.388 | 1.00 | 40.65 | C |
| ATOM | 7288 | CB | HIS | C | 10 | −1.874 | 11.593 | 20.618 | 1.00 | 37.61 | C |
| ATOM | 7289 | CG | HIS | C | 10 | −1.012 | 10.389 | 20.380 | 1.00 | 39.10 | C |
| ATOM | 7290 | CD2 | HIS | C | 10 | −0.301 | 10.004 | 19.294 | 1.00 | 36.83 | C |
| ATOM | 7291 | ND1 | HIS | C | 10 | −0.759 | 9.446 | 21.355 | 1.00 | 39.24 | C |
| ATOM | 7292 | CE1 | HIS | C | 10 | 0.074 | 8.536 | 20.881 | 1.00 | 39.59 | C |
| ATOM | 7293 | NE2 | HIS | C | 10 | 0.368 | 8.852 | 19.631 | 1.00 | 37.07 | C |
| ATOM | 7294 | C | HIS | C | 10 | −0.063 | 13.290 | 20.531 | 1.00 | 38.86 | C |
| ATOM | 7295 | O | HIS | C | 10 | −0.217 | 13.430 | 19.319 | 1.00 | 37.02 | C |
| ATOM | 7296 | N | VAL | C | 11 | 1.037 | 13.670 | 21.160 | 1.00 | 49.46 | C |
| ATOM | 7297 | CA | VAL | C | 11 | 2.141 | 14.260 | 20.423 | 1.00 | 52.04 | C |
| ATOM | 7298 | CB | VAL | C | 11 | 2.713 | 15.470 | 21.155 | 1.00 | 36.64 | C |
| ATOM | 7299 | CG1 | VAL | C | 11 | 1.629 | 16.523 | 21.290 | 1.00 | 32.70 | C |
| ATOM | 7300 | CG2 | VAL | C | 11 | 3.240 | 15.055 | 22.538 | 1.00 | 38.62 | C |
| ATOM | 7301 | C | VAL | C | 11 | 3.216 | 13.226 | 20.211 | 1.00 | 50.65 | C |
| ATOM | 7302 | O | VAL | C | 11 | 3.367 | 12.285 | 20.992 | 1.00 | 47.66 | C |
| ATOM | 7303 | N | VAL | C | 12 | 3.966 | 13.407 | 19.141 | 1.00 | 48.56 | C |
| ATOM | 7304 | CA | VAL | C | 12 | 4.998 | 12.464 | 18.793 | 1.00 | 46.73 | C |
| ATOM | 7305 | CB | VAL | C | 12 | 4.605 | 11.790 | 17.476 | 1.00 | 58.63 | C |
| ATOM | 7306 | CG1 | VAL | C | 12 | 4.788 | 12.754 | 16.340 | 1.00 | 52.43 | C |
| ATOM | 7307 | CG2 | VAL | C | 12 | 5.392 | 10.518 | 17.273 | 1.00 | 62.10 | C |
| ATOM | 7308 | C | VAL | C | 12 | 6.359 | 13.169 | 18.690 | 1.00 | 44.69 | C |
| ATOM | 7309 | O | VAL | C | 12 | 6.431 | 14.397 | 18.578 | 1.00 | 44.53 | C |
| ATOM | 7310 | N | ALA | C | 13 | 7.434 | 12.391 | 18.750 | 1.00 | 52.14 | C |
| ATOM | 7311 | CA | ALA | C | 13 | 8.788 | 12.929 | 18.692 | 1.00 | 51.81 | C |
| ATOM | 7312 | CB | ALA | C | 13 | 9.775 | 11.889 | 19.191 | 1.00 | 37.22 | C |
| ATOM | 7313 | C | ALA | C | 13 | 9.200 | 13.390 | 17.306 | 1.00 | 55.79 | C |
| ATOM | 7314 | O | ALA | C | 13 | 8.909 | 12.727 | 16.309 | 1.00 | 53.30 | C |
| ATOM | 7315 | N | ASN | C | 14 | 9.896 | 14.525 | 17.262 | 1.00 | 48.38 | C |
| ATOM | 7316 | CA | ASN | C | 14 | 10.386 | 15.114 | 16.019 | 1.00 | 49.50 | C |
| ATOM | 7317 | CB | ASN | C | 14 | 10.631 | 16.605 | 16.224 | 1.00 | 74.27 | C |
| ATOM | 7318 | CG | ASN | C | 14 | 11.137 | 17.291 | 14.974 | 1.00 | 76.26 | C |
| ATOM | 7319 | OD1 | ASN | C | 14 | 11.447 | 18.483 | 14.997 | 1.00 | 75.84 | C |
| ATOM | 7320 | ND2 | ASN | C | 14 | 11.218 | 16.546 | 13.872 | 1.00 | 78.38 | C |
| ATOM | 7321 | C | ASN | C | 14 | 11.690 | 14.441 | 15.618 | 1.00 | 52.38 | C |
| ATOM | 7322 | O | ASN | C | 14 | 12.696 | 14.564 | 16.323 | 1.00 | 52.90 | C |
| ATOM | 7323 | N | PRO | C | 15 | 11.699 | 13.731 | 14.474 | 1.00 | 67.74 | C |
| ATOM | 7324 | CD | PRO | C | 15 | 10.597 | 13.499 | 13.525 | 1.00 | 34.13 | C |
| ATOM | 7325 | CA | PRO | C | 15 | 12.920 | 13.049 | 14.023 | 1.00 | 71.34 | C |
| ATOM | 7326 | CB | PRO | C | 15 | 12.418 | 12.098 | 12.928 | 1.00 | 38.47 | C |
| ATOM | 7327 | CG | PRO | C | 15 | 10.901 | 12.108 | 13.061 | 1.00 | 36.74 | C |
| ATOM | 7328 | C | PRO | C | 15 | 13.961 | 14.020 | 13.476 | 1.00 | 72.55 | C |
| ATOM | 7329 | O | PRO | C | 15 | 15.145 | 13.715 | 13.433 | 1.00 | 74.58 | C |
| ATOM | 7330 | N | GLN | C | 16 | 13.500 | 15.191 | 13.061 | 1.00 | 97.37 | C |
| ATOM | 7331 | CA | GLN | C | 16 | 14.373 | 16.201 | 12.487 | 1.00 | 96.32 | C |
| ATOM | 7332 | CB | GLN | C | 16 | 13.532 | 17.183 | 11.659 | 1.00 | 124.05 | C |
| ATOM | 7333 | CG | GLN | C | 16 | 14.307 | 17.990 | 10.623 | 1.00 | 126.98 | C |
| ATOM | 7334 | CD | GLN | C | 16 | 15.011 | 17.110 | 9.602 | 1.00 | 128.77 | C |
| ATOM | 7335 | OE1 | GLN | C | 16 | 14.441 | 16.136 | 9.111 | 1.00 | 130.89 | C |
| ATOM | 7336 | NE2 | GLN | C | 16 | 16.252 | 17.459 | 9.269 | 1.00 | 127.42 | C |
| ATOM | 7337 | C | GLN | C | 16 | 15.166 | 16.955 | 13.557 | 1.00 | 92.99 | C |
| ATOM | 7338 | O | GLN | C | 16 | 15.925 | 17.876 | 13.248 | 1.00 | 93.56 | C |
| ATOM | 7339 | N | ALA | C | 17 | 14.996 | 16.565 | 14.815 | 1.00 | 89.06 | C |
| ATOM | 7340 | CA | ALA | C | 17 | 15.704 | 17.229 | 15.906 | 1.00 | 92.82 | C |
| ATOM | 7341 | CB | ALA | C | 17 | 14.706 | 17.828 | 16.888 | 1.00 | 39.49 | C |
| ATOM | 7342 | C | ALA | C | 17 | 16.623 | 16.253 | 16.623 | 1.00 | 97.32 | C |
| ATOM | 7343 | O | ALA | C | 17 | 16.448 | 15.968 | 17.807 | 1.00 | 98.96 | C |
| ATOM | 7344 | N | GLU | C | 18 | 17.608 | 15.747 | 15.892 | 1.00 | 89.80 | C |
| ATOM | 7345 | CA | GLU | C | 18 | 18.561 | 14.793 | 16.440 | 1.00 | 91.80 | C |
| ATOM | 7346 | CB | GLU | C | 18 | 19.701 | 14.566 | 15.446 | 1.00 | 125.96 | C |
| ATOM | 7347 | CG | GLU | C | 18 | 20.502 | 15.820 | 15.134 | 1.00 | 125.96 | C |
| ATOM | 7348 | CD | GLU | C | 13 | 21.705 | 15.533 | 14.260 | 1.00 | 125.96 | C |
| ATOM | 7349 | OE1 | GLU | C | 18 | 22.584 | 14.761 | 14.696 | 1.00 | 125.96 | C |
| ATOM | 7350 | OE2 | GLU | C | 18 | 21.771 | 16.077 | 13.138 | 1.00 | 125.96 | C |
| ATOM | 7351 | C | GLU | C | 18 | 19.148 | 15.241 | 17.776 | 1.00 | 91.46 | C |
| ATOM | 7352 | O | GLU | C | 18 | 19.309 | 16.437 | 18.036 | 1.00 | 91.07 | C |
| ATOM | 7353 | N | GLY | C | 19 | 19.459 | 14.261 | 18.619 | 1.00 | 93.98 | C |
| ATOM | 7354 | CA | GLY | C | 19 | 20.054 | 14.542 | 19.911 | 1.00 | 91.47 | C |
| ATOM | 7355 | C | GLY | C | 19 | 19.118 | 15.009 | 21.003 | 1.00 | 89.97 | C |
| ATOM | 7356 | O | GLY | C | 19 | 19.527 | 15.108 | 22.155 | 1.00 | 89.98 | C |
| ATOM | 7357 | N | GLN | C | 20 | 17.867 | 15.297 | 20.663 | 1.00 | 94.73 | C |
| ATOM | 7358 | CA | GLN | C | 20 | 16.916 | 15.758 | 21.667 | 1.00 | 90.30 | C |
| ATOM | 7359 | CB | GLN | C | 20 | 16.853 | 17.280 | 21.648 | 1.00 | 114.59 | C |
| ATOM | 7360 | CG | GLN | C | 20 | 16.556 | 17.846 | 20.281 | 1.00 | 115.57 | C |
| ATOM | 7361 | CD | GLN | C | 20 | 16.571 | 19.360 | 20.269 | 1.00 | 115.33 | C |
| ATOM | 7362 | OE1 | GLN | C | 20 | 17.584 | 19.981 | 20.593 | 1.00 | 114.98 | C |
| ATOM | 7363 | NE2 | GLN | C | 20 | 15.445 | 19.966 | 19.897 | 1.00 | 114.78 | C |
| ATOM | 7364 | C | GLN | C | 20 | 15.519 | 15.179 | 21.476 | 1.00 | 85.04 | C |
| ATOM | 7365 | O | GLN | C | 20 | 15.225 | 14.553 | 20.453 | 1.00 | 83.75 | C |
| ATOM | 7366 | N | LEU | C | 21 | 14.662 | 15.391 | 22.471 | 1.00 | 59.03 | C |
| ATOM | 7367 | CA | LEU | C | 21 | 13.296 | 14.887 | 22.420 | 1.00 | 55.49 | C |
| ATOM | 7368 | CB | LEU | C | 21 | 13.006 | 14.017 | 23.643 | 1.00 | 53.15 | C |
| ATOM | 7369 | CG | LEU | C | 21 | 11.597 | 13.447 | 23.804 | 1.00 | 51.93 | C |
| ATOM | 7370 | CD1 | LEU | C | 21 | 11.141 | 12.792 | 22.502 | 1.00 | 49.23 | C |
| ATOM | 7371 | CD2 | LEU | C | 21 | 11.589 | 12.447 | 24.956 | 1.00 | 53.97 | C |
| ATOM | 7372 | C | LEU | C | 21 | 12.318 | 16.039 | 22.366 | 1.00 | 54.10 | C |
| ATOM | 7373 | O | LEU | C | 21 | 11.900 | 16.553 | 23.398 | 1.00 | 49.42 | C |
| ATOM | 7374 | N | GLN | C | 22 | 11.954 | 16.442 | 21.156 | 1.00 | 65.98 | C |
| ATOM | 7375 | CA | GLN | C | 22 | 11.026 | 17.546 | 20.961 | 1.00 | 70.58 | C |
| ATOM | 7376 | CB | GLN | C | 22 | 11.634 | 18.516 | 19.957 | 1.00 | 74.86 | C |
| ATOM | 7377 | CG | GLN | C | 22 | 10.721 | 19.620 | 19.497 | 1.00 | 81.63 | C |
| ATOM | 7378 | CD | GLN | C | 22 | 11.273 | 20.334 | 18.279 | 1.00 | 87.85 | C |
| ATOM | 7379 | OE1 | GLN | C | 22 | 10.606 | 21.185 | 17.690 | 1.00 | 93.08 | C |
| ATOM | 7380 | NE2 | GLN | C | 22 | 12.499 | 19.990 | 17.893 | 1.00 | 88.61 | C |
| ATOM | 7381 | C | GLN | C | 22 | 9.678 | 17.010 | 20.460 | 1.00 | 67.97 | C |
| ATOM | 7382 | O | GLN | C | 22 | 9.621 | 16.294 | 19.464 | 1.00 | 66.92 | C |
| ATOM | 7383 | N | TRP | C | 23 | 8.596 | 17.361 | 21.151 | 1.00 | 46.70 | C |
| ATOM | 7384 | CA | TRP | C | 23 | 7.263 | 16.887 | 20.786 | 1.00 | 40.58 | C |
| ATOM | 7385 | CB | TRP | C | 23 | 6.360 | 16.801 | 22.024 | 1.00 | 42.59 | C |
| ATOM | 7386 | CG | TRP | C | 23 | 6.893 | 15.977 | 23.149 | 1.00 | 43.11 | C |
| ATOM | 7387 | CD2 | TRP | C | 23 | 7.070 | 14.553 | 23.168 | 1.00 | 41.22 | C |
| ATOM | 7388 | CE2 | TRP | C | 23 | 7.604 | 14.216 | 24.437 | 1.00 | 42.84 | C |
| ATOM | 7389 | CE3 | TRP | C | 23 | 6.832 | 13.528 | 22.238 | 1.00 | 40.91 | C |
| ATOM | 7390 | CD1 | TRP | C | 23 | 7.312 | 16.431 | 24.364 | 1.00 | 42.58 | C |
| ATOM | 7391 | NE1 | TRP | C | 23 | 7.740 | 15.379 | 25.147 | 1.00 | 41.67 | C |
| ATOM | 7392 | CZ2 | TRP | C | 23 | 7.903 | 12.897 | 24.798 | 1.00 | 44.90 | C |
| ATOM | 7393 | CZ3 | TRP | C | 23 | 7.129 | 12.213 | 22.601 | 1.00 | 45.35 | C |
| ATOM | 7394 | CH2 | TRP | C | 23 | 7.659 | 11.914 | 23.870 | 1.00 | 43.16 | C |
| ATOM | 7395 | C | TRP | C | 23 | 6.543 | 17.754 | 19.765 | 1.00 | 41.08 | C |
| ATOM | 7396 | O | TRP | C | 23 | 6.570 | 18.982 | 19.857 | 1.00 | 42.36 | C |
| ATOM | 7397 | N | LEU | C | 24 | 5.893 | 17.115 | 18.796 | 1.00 | 38.90 | C |
| ATOM | 7398 | CA | LEU | C | 24 | 5.130 | 17.868 | 17.816 | 1.00 | 36.59 | C |
| ATOM | 7399 | CB | LEU | C | 24 | 5.792 | 17.895 | 16.430 | 1.00 | 62.51 | C |
| ATOM | 7400 | CG | LEU | C | 24 | 7.235 | 17.493 | 16.208 | 1.00 | 63.24 | C |
| ATOM | 7401 | CD1 | LEU | C | 24 | 7.341 | 15.995 | 16.353 | 1.00 | 64.86 | C |
| ATOM | 7402 | CD2 | LEU | C | 24 | 7.665 | 17.902 | 14.824 | 1.00 | 58.35 | C |
| ATOM | 7403 | C | LEU | C | 24 | 3.757 | 17.254 | 17.675 | 1.00 | 43.45 | C |
| ATOM | 7404 | O | LEU | C | 24 | 3.542 | 16.088 | 18.007 | 1.00 | 40.43 | C |
| ATOM | 7405 | N | ASN | C | 25 | 2.830 | 18.054 | 17.167 | 1.00 | 48.15 | C |
| ATOM | 7406 | CA | ASN | C | 25 | 1.473 | 17.604 | 16.961 | 1.00 | 49.59 | C |
| ATOM | 7407 | CB | ASN | C | 25 | 0.537 | 18.439 | 17.821 | 1.00 | 44.81 | C |
| ATOM | 7408 | CG | ASN | C | 25 | 0.376 | 19.839 | 17.294 | 1.00 | 46.38 | C |
| ATOM | 7409 | OD1 | ASN | C | 25 | −0.713 | 20.226 | 16.858 | 1.00 | 50.37 | C |
| ATOM | 7410 | ND2 | ASN | C | 25 | 1.464 | 20.611 | 17.317 | 1.00 | 42.78 | C |
| ATOM | 7411 | C | ASN | C | 25 | 1.086 | 17.766 | 15.495 | 1.00 | 50.12 | C |
| ATOM | 7412 | O | ASN | C | 25 | −0.016 | 17.399 | 15.100 | 1.00 | 52.48 | C |
| ATOM | 7413 | N | ARG | C | 26 | 1.986 | 18.317 | 14.688 | 1.00 | 57.30 | C |
| ATOM | 7414 | CA | ARG | C | 26 | 1.692 | 18.547 | 13.279 | 1.00 | 58.58 | C |
| ATOM | 7415 | CB | ARG | C | 26 | 2.452 | 19.776 | 12.776 | 1.00 | 43.45 | C |
| ATOM | 7416 | CG | ARG | C | 26 | 3.944 | 19.722 | 13.000 | 1.00 | 44.36 | C |
| ATOM | 7417 | CD | ARG | C | 26 | 4.630 | 20.821 | 12.230 | 1.00 | 48.42 | C |
| ATOM | 7418 | NE | ARG | C | 26 | 6.075 | 20.817 | 12.424 | 1.00 | 49.56 | C |
| ATOM | 7419 | CZ | ARG | C | 26 | 6.689 | 21.378 | 13.461 | 1.00 | 50.12 | C |
| ATOM | 7420 | NH1 | ARG | C | 26 | 5.987 | 21.995 | 14.402 | 1.00 | 50.60 | C |
| ATOM | 7421 | NH2 | ARG | C | 26 | 8.010 | 21.317 | 13.559 | 1.00 | 49.15 | C |
| ATOM | 7422 | C | ARG | C | 26 | 2.009 | 17.365 | 12.384 | 1.00 | 56.64 | C |
| ATOM | 7423 | O | ARG | C | 26 | 2.615 | 17.530 | 11.324 | 1.00 | 56.35 | C |
| ATOM | 7424 | N | ARG | C | 27 | 1.595 | 16.172 | 12.797 | 1.00 | 43.67 | C |
| ATOM | 7425 | CA | ARG | C | 27 | 1.859 | 14.988 | 11.996 | 1.00 | 44.60 | C |
| ATOM | 7426 | CB | ARG | C | 27 | 2.995 | 14.173 | 12.611 | 1.00 | 48.71 | C |
| ATOM | 7427 | CG | ARG | C | 27 | 4.325 | 14.870 | 12.553 | 1.00 | 50.13 | C |
| ATOM | 7428 | CD | ARG | C | 27 | 4.616 | 15.325 | 11.140 | 1.00 | 52.50 | C |
| ATOM | 7429 | NE | ARG | C | 27 | 5.844 | 16.112 | 11.036 | 1.00 | 55.89 | C |
| ATOM | 7430 | CZ | ARG | C | 27 | 7.061 | 15.647 | 11.315 | 1.00 | 59.50 | C |
| ATOM | 7431 | NH1 | ARG | C | 27 | 8.119 | 16.444 | 11.188 | 1.00 | 60.37 | C |
| ATOM | 7432 | NH2 | ARG | C | 27 | 7.223 | 14.385 | 11.722 | 1.00 | 57.73 | C |
| ATOM | 7433 | C | ARG | C | 27 | 0.649 | 14.096 | 11.769 | 1.00 | 45.06 | C |
| ATOM | 7434 | O | ARG | C | 27 | −0.434 | 14.332 | 12.311 | 1.00 | 45.20 | C |
| ATOM | 7435 | N | ALA | C | 28 | 0.879 | 13.074 | 10.948 | 1.00 | 79.27 | C |
| ATOM | 7436 | CA | ALA | C | 28 | −0.104 | 12.079 | 10.561 | 1.00 | 78.17 | C |
| ATOM | 7437 | CB | ALA | C | 28 | 0.601 | 10.773 | 10.235 | 1.00 | 131.19 | C |
| ATOM | 7438 | C | ALA | C | 28 | −1.212 | 11.814 | 11.559 | 1.00 | 77.83 | C |
| ATOM | 7439 | O | ALA | C | 28 | −2.294 | 12.390 | 11.470 | 1.00 | 78.85 | C |
| ATOM | 7440 | N | ASN | C | 29 | −0.955 | 10.934 | 12.513 | 1.00 | 46.48 | C |
| ATOM | 7441 | CA | ASN | C | 29 | −2.000 | 10.598 | 13.469 | 1.00 | 45.65 | C |
| ATOM | 7442 | CB | ASN | C | 29 | −2.184 | 9.083 | 13.498 | 1.00 | 50.09 | C |
| ATOM | 7443 | CG | ASN | C | 29 | −2.648 | 8.529 | 12.162 | 1.00 | 50.15 | C |
| ATOM | 7444 | OD1 | ASN | C | 29 | −3.836 | 8.588 | 11.825 | 1.00 | 56.01 | C |
| ATOM | 7445 | ND2 | ASN | C | 29 | −1.704 | 8.000 | 11.387 | 1.00 | 47.16 | C |
| ATOM | 7446 | C | ASN | C | 29 | −1.705 | 11.125 | 14.858 | 1.00 | 43.99 | C |
| ATOM | 7447 | O | ASN | C | 29 | −1.602 | 10.361 | 15.809 | 1.00 | 41.71 | C |
| ATOM | 7448 | N | ALA | C | 30 | −1.572 | 12.441 | 14.964 | 1.00 | 37.32 | C |
| ATOM | 7449 | CA | ALA | C | 30 | −1.283 | 13.089 | 16.231 | 1.00 | 32.14 | C |
| ATOM | 7450 | CB | ALA | C | 30 | −0.016 | 13.893 | 16.119 | 1.00 | 33.14 | C |
| ATOM | 7451 | C | ALA | C | 30 | −2.450 | 13.981 | 16.621 | 1.00 | 33.54 | C |
| ATOM | 7452 | O | ALA | C | 30 | −3.334 | 14.253 | 15.798 | 1.00 | 37.69 | C |
| ATOM | 7453 | N | LEU | C | 31 | −2.456 | 14.429 | 17.874 | 1.00 | 40.27 | C |
| ATOM | 7454 | CA | LEU | C | 31 | −3.544 | 15.258 | 18.365 | 1.00 | 39.74 | C |
| ATOM | 7455 | CB | LEU | C | 31 | −4.582 | 14.388 | 19.060 | 1.00 | 39.42 | C |
| ATOM | 7456 | CG | LEU | C | 31 | −5.500 | 13.485 | 18.257 | 1.00 | 42.30 | C |
| ATOM | 7457 | CD1 | LEU | C | 31 | −6.184 | 12.516 | 19.197 | 1.00 | 39.92 | C |
| ATOM | 7458 | CD2 | LEU | C | 31 | −6.511 | 14.322 | 17.523 | 1.00 | 41.77 | C |
| ATOM | 7459 | C | LEU | C | 31 | −3.124 | 16.328 | 19.352 | 1.00 | 39.00 | C |
| ATOM | 7460 | O | LEU | C | 31 | −2.193 | 16.139 | 20.130 | 1.00 | 39.81 | C |
| ATOM | 7461 | N | LEU | C | 32 | −3.855 | 17.436 | 19.330 | 1.00 | 51.05 | C |
| ATOM | 7462 | CA | LEU | C | 32 | −3.647 | 18.557 | 20.233 | 1.00 | 50.84 | C |
| ATOM | 7463 | CB | LEU | C | 32 | −2.442 | 19.395 | 19.810 | 1.00 | 43.32 | C |
| ATOM | 7464 | CG | LEU | C | 32 | −2.159 | 20.519 | 20.807 | 1.00 | 41.90 | C |
| ATOM | 7465 | CD1 | LEU | C | 32 | −1.721 | 19.895 | 22.131 | 1.00 | 40.88 | C |
| ATOM | 7466 | CD2 | LEU | C | 32 | −1.087 | 21.436 | 20.274 | 1.00 | 42.22 | C |
| ATOM | 7467 | C | LEU | C | 32 | −4.914 | 19.376 | 20.101 | 1.00 | 50.48 | C |
| ATOM | 7468 | O | LEU | C | 32 | −4.959 | 20.340 | 19.345 | 1.00 | 56.35 | C |
| ATOM | 7469 | N | ALA | C | 33 | −5.946 | 18.997 | 20.842 | 1.00 | 29.32 | C |
| ATOM | 7470 | CA | ALA | C | 33 | −7.223 | 19.686 | 20.734 | 1.00 | 29.84 | C |
| ATOM | 7471 | CB | ALA | C | 33 | −8.116 | 18.911 | 19.814 | 1.00 | 31.53 | C |
| ATOM | 7472 | C | ALA | C | 33 | −7.995 | 20.004 | 21.997 | 1.00 | 31.63 | C |
| ATOM | 7473 | O | ALA | C | 33 | −7.655 | 19.594 | 23.105 | 1.00 | 35.81 | C |
| ATOM | 7474 | N | ASN | C | 34 | −9.064 | 20.755 | 21.787 | 1.00 | 47.19 | C |
| ATOM | 7475 | CA | ASN | C | 34 | −9.960 | 21.158 | 22.846 | 1.00 | 44.38 | C |
| ATOM | 7476 | CB | ASN | C | 34 | −10.797 | 19.967 | 23.272 | 1.00 | 57.33 | C |
| ATOM | 7477 | CG | ASN | C | 34 | −11.591 | 19.415 | 22.133 | 1.00 | 61.25 | C |
| ATOM | 7478 | OD1 | ASN | C | 34 | −12.275 | 20.159 | 21.430 | 1.00 | 63.73 | C |
| ATOM | 7479 | ND2 | ASN | C | 34 | −11.508 | 18.107 | 21.930 | 1.00 | 56.66 | C |
| ATOM | 7480 | C | ASN | C | 34 | −9.342 | 21.810 | 24.064 | 1.00 | 45.13 | C |
| ATOM | 7481 | O | ASN | C | 34 | −9.741 | 21.525 | 25.195 | 1.00 | 47.67 | C |
| ATOM | 7482 | N | GLY | C | 35 | −8.371 | 22.686 | 23.835 | 1.00 | 49.66 | C |
| ATOM | 7483 | CA | GLY | C | 35 | −7.773 | 23.390 | 24.947 | 1.00 | 44.25 | C |
| ATOM | 7484 | C | GLY | C | 35 | −6.376 | 23.046 | 25.402 | 1.00 | 44.60 | C |
| ATOM | 7485 | O | GLY | C | 35 | −5.659 | 23.931 | 25.857 | 1.00 | 46.09 | C |
| ATOM | 7486 | N | VAL | C | 36 | −5.966 | 21.792 | 25.323 | 1.00 | 36.54 | C |
| ATOM | 7487 | CA | VAL | C | 36 | −4.620 | 21.506 | 25.784 | 1.00 | 37.87 | C |
| ATOM | 7488 | CB | VAL | C | 36 | −4.367 | 19.968 | 25.958 | 1.00 | 38.93 | C |
| ATOM | 7489 | CG1 | VAL | C | 36 | −5.512 | 19.184 | 25.390 | 1.00 | 36.96 | C |
| ATOM | 7490 | CG2 | VAL | C | 36 | −3.060 | 19.561 | 25.327 | 1.00 | 39.33 | C |
| ATOM | 7491 | C | VAL | C | 36 | −3.616 | 22.163 | 24.841 | 1.00 | 42.02 | C |
| ATOM | 7492 | O | VAL | C | 36 | −3.791 | 22.173 | 23.625 | 1.00 | 42.98 | C |
| ATOM | 7493 | N | GLU | C | 37 | −2.575 | 22.743 | 25.426 | 1.00 | 51.77 | C |
| ATOM | 7494 | CA | GLU | C | 37 | −1.563 | 23.432 | 24.656 | 1.00 | 54.37 | C |
| ATOM | 7495 | CB | GLU | C | 37 | −1.459 | 24.878 | 25.132 | 1.00 | 68.20 | C |
| ATOM | 7496 | CG | GLU | C | 37 | −2.802 | 25.484 | 25.475 | 1.00 | 75.23 | C |
| ATOM | 7497 | CD | GLU | C | 37 | −2.689 | 26.777 | 26.269 | 1.00 | 78.20 | C |
| ATOM | 7498 | OE1 | GLU | C | 37 | −1.698 | 26.938 | 27.024 | 1.00 | 81.63 | C |
| ATOM | 7499 | OE2 | GLU | C | 37 | −3.606 | 27.625 | 26.155 | 1.00 | 78.76 | C |
| ATOM | 7500 | C | GLU | C | 37 | −0.220 | 22.758 | 24.807 | 1.00 | 53.26 | C |
| ATOM | 7501 | O | GLU | C | 37 | 0.045 | 22.105 | 25.807 | 1.00 | 55.72 | C |
| ATOM | 7502 | N | LEU | C | 38 | 0.619 | 22.909 | 23.791 | 1.00 | 61.71 | C |
| ATOM | 7503 | CA | LEU | C | 38 | 1.958 | 22.352 | 23.822 | 1.00 | 63.75 | C |
| ATOM | 7504 | CB | LEU | C | 38 | 2.262 | 21.609 | 22.520 | 1.00 | 35.53 | C |
| ATOM | 7505 | CG | LEU | C | 38 | 3.609 | 20.892 | 22.459 | 1.00 | 33.44 | C |
| ATOM | 7506 | CD1 | LEU | C | 38 | 3.802 | 20.073 | 23.727 | 1.00 | 31.82 | C |
| ATOM | 7507 | CD2 | LEU | C | 38 | 3.671 | 20.013 | 21.219 | 1.00 | 30.31 | C |
| ATOM | 7508 | C | LEU | C | 38 | 2.816 | 23.599 | 23.954 | 1.00 | 64.02 | C |
| ATOM | 7509 | O | LEU | C | 38 | 3.002 | 24.338 | 22.991 | 1.00 | 57.63 | C |
| ATOM | 7510 | N | ARG | C | 39 | 3.307 | 23.835 | 25.165 | 1.00 | 44.58 | C |
| ATOM | 7511 | CA | ARG | C | 39 | 4.105 | 25.013 | 25.479 | 1.00 | 51.11 | C |
| ATOM | 7512 | CB | ARG | C | 39 | 3.304 | 25.883 | 26.443 | 1.00 | 57.55 | C |
| ATOM | 7513 | CG | ARG | C | 39 | 3.847 | 27.244 | 26.692 | 1.00 | 65.25 | C |
| ATOM | 7514 | CD | ARG | C | 39 | 2.816 | 28.070 | 27.434 | 1.00 | 73.86 | C |
| ATOM | 7515 | NE | ARG | C | 39 | 3.174 | 29.484 | 27.416 | 1.00 | 80.39 | C |
| ATOM | 7516 | CZ | ARG | C | 39 | 2.351 | 30.469 | 27.763 | 1.00 | 81.19 | C |
| ATOM | 7517 | NH1 | ARG | C | 39 | 1.116 | 30.191 | 28.159 | 1.00 | 79.67 | C |
| ATOM | 7518 | NH2 | ARG | C | 39 | 2.756 | 31.734 | 27.704 | 1.00 | 83.49 | C |
| ATOM | 7519 | C | ARG | C | 39 | 5.446 | 24.618 | 26.098 | 1.00 | 53.16 | C |
| ATOM | 7520 | O | ARG | C | 39 | 5.506 | 23.807 | 27.022 | 1.00 | 56.39 | C |
| ATOM | 7521 | N | ASP | C | 40 | 6.521 | 25.198 | 25.584 | 1.00 | 51.02 | C |
| ATOM | 7522 | CA | ASP | C | 40 | 7.862 | 24.903 | 26.076 | 1.00 | 48.97 | C |
| ATOM | 7523 | CB | ASP | C | 40 | 8.085 | 25.581 | 27.425 | 1.00 | 90.16 | C |
| ATOM | 7524 | CG | ASP | C | 40 | 7.826 | 27.078 | 27.373 | 1.00 | 92.30 | C |
| ATOM | 7525 | OD1 | ASP | C | 40 | 8.417 | 27.755 | 26.504 | 1.00 | 89.18 | C |
| ATOM | 7526 | OD2 | ASP | C | 40 | 7.031 | 27.582 | 28.198 | 1.00 | 92.42 | C |
| ATOM | 7527 | C | ASP | C | 40 | 8.088 | 23.395 | 26.194 | 1.00 | 45.10 | C |
| ATOM | 7528 | O | ASP | C | 40 | 8.716 | 22.933 | 27.136 | 1.00 | 44.33 | C |
| ATOM | 7529 | N | ASN | C | 41 | 7.557 | 22.637 | 25.235 | 1.00 | 43.89 | C |
| ATOM | 7530 | CA | ASN | C | 41 | 7.696 | 21.182 | 25.175 | 1.00 | 39.64 | C |
| ATOM | 7531 | CB | ASN | C | 41 | 9.173 | 20.786 | 25.220 | 1.00 | 60.33 | C |
| ATOM | 7532 | CG | ASN | C | 41 | 9.443 | 19.456 | 24.535 | 1.00 | 55.02 | C |
| ATOM | 7533 | OD1 | ASN | C | 41 | 9.098 | 19.264 | 23.368 | 1.00 | 56.36 | C |
| ATOM | 7534 | ND2 | ASN | C | 41 | 10.068 | 18.536 | 25.253 | 1.00 | 51.12 | C |
| ATOM | 7535 | C | ASN | C | 41 | 6.937 | 20.432 | 26.261 | 1.00 | 42.95 | C |
| ATOM | 7536 | O | ASN | C | 41 | 7.285 | 19.302 | 26.611 | 1.00 | 44.46 | C |
| ATOM | 7537 | N | GLN | C | 42 | 5.892 | 21.056 | 26.789 | 1.00 | 49.84 | C |
| ATOM | 7538 | CA | GLN | C | 42 | 5.096 | 20.429 | 27.830 | 1.00 | 47.76 | C |
| ATOM | 7539 | CB | GLN | C | 42 | 5.359 | 21.087 | 29.183 | 1.00 | 40.52 | C |
| ATOM | 7540 | CG | GLN | C | 42 | 6.816 | 21.249 | 29.505 | 1.00 | 42.61 | C |
| ATOM | 7541 | CD | GLN | C | 42 | 7.048 | 21.980 | 30.816 | 1.00 | 42.07 | C |
| ATOM | 7542 | OE1 | GLN | C | 42 | 6.447 | 23.030 | 31.084 | 1.00 | 41.50 | C |
| ATOM | 7543 | NE2 | GLN | C | 42 | 7.940 | 21.435 | 31.635 | 1.00 | 43.83 | C |
| ATOM | 7544 | C | GLN | C | 42 | 3.637 | 20.595 | 27.481 | 1.00 | 46.65 | C |
| ATOM | 7545 | O | GLN | C | 42 | 3.250 | 21.586 | 26.861 | 1.00 | 46.99 | C |
| ATOM | 7546 | N | LEU | C | 43 | 2.826 | 19.623 | 27.875 | 1.00 | 39.15 | C |
| ATOM | 7547 | CA | LEU | C | 43 | 1.400 | 19.698 | 27.615 | 1.00 | 37.73 | C |
| ATOM | 7548 | CB | LEU | C | 43 | 0.808 | 18.281 | 27.533 | 1.00 | 30.38 | C |
| ATOM | 7549 | CG | LEU | C | 43 | 1.326 | 17.440 | 26.373 | 1.00 | 36.54 | C |
| ATOM | 7550 | CD1 | LEU | C | 43 | 0.848 | 16.021 | 26.494 | 1.00 | 36.63 | C |
| ATOM | 7551 | CD2 | LEU | C | 43 | 0.863 | 18.067 | 25.071 | 1.00 | 32.93 | C |
| ATOM | 7552 | C | LEU | C | 43 | 0.776 | 20.495 | 28.766 | 1.00 | 39.75 | C |
| ATOM | 7553 | O | LEU | C | 43 | 1.138 | 20.309 | 29.930 | 1.00 | 36.37 | C |
| ATOM | 7554 | N | VAL | C | 44 | −0.150 | 21.389 | 28.450 | 1.00 | 33.75 | C |
| ATOM | 7555 | CA | VAL | C | 44 | −0.784 | 22.176 | 29.494 | 1.00 | 33.43 | C |
| ATOM | 7556 | CB | VAL | C | 44 | −0.540 | 23.659 | 29.280 | 1.00 | 32.17 | C |
| ATOM | 7557 | CG1 | VAL | C | 44 | −1.061 | 24.425 | 30.460 | 1.00 | 33.36 | C |
| ATOM | 7558 | CG2 | VAL | C | 44 | 0.943 | 23.912 | 29.062 | 1.00 | 26.78 | C |
| ATOM | 7559 | C | VAL | C | 44 | −2.286 | 21.919 | 29.550 | 1.00 | 32.44 | C |
| ATOM | 7560 | O | VAL | C | 44 | −3.010 | 22.149 | 28.581 | 1.00 | 33.48 | C |
| ATOM | 7561 | N | VAL | C | 45 | −2.739 | 21.433 | 30.703 | 1.00 | 38.65 | C |
| ATOM | 7562 | CA | VAL | C | 45 | −4.135 | 21.094 | 30.925 | 1.00 | 44.39 | C |
| ATOM | 7563 | CB | VAL | C | 45 | −4.274 | 20.260 | 32.212 | 1.00 | 52.22 | C |
| ATOM | 7564 | CG1 | VAL | C | 45 | −5.721 | 20.136 | 32.603 | 1.00 | 54.79 | C |
| ATOM | 7565 | CG2 | VAL | C | 45 | −3.697 | 18.881 | 31.986 | 1.00 | 55.62 | C |
| ATOM | 7566 | C | VAL | C | 45 | −5.012 | 22.336 | 31.002 | 1.00 | 46.18 | C |
| ATOM | 7567 | O | VAL | C | 45 | −4.687 | 23.290 | 31.692 | 1.00 | 45.63 | C |
| ATOM | 7568 | N | PRO | C | 46 | −6.147 | 22.329 | 30.285 | 1.00 | 40.92 | C |
| ATOM | 7569 | CD | PRO | C | 46 | −6.475 | 21.359 | 29.219 | 1.00 | 28.72 | C |
| ATOM | 7570 | CA | PRO | C | 46 | −7.082 | 23.461 | 30.264 | 1.00 | 39.77 | C |
| ATOM | 7571 | CB | PRO | C | 46 | −7.645 | 23.408 | 28.856 | 1.00 | 28.08 | C |
| ATOM | 7572 | CG | PRO | C | 46 | −7.773 | 21.927 | 28.636 | 1.00 | 28.78 | C |
| ATOM | 7573 | C | PRO | C | 46 | −8.198 | 23.453 | 31.286 | 1.00 | 43.21 | C |
| ATOM | 7574 | O | PRO | C | 46 | −8.947 | 24.415 | 31.358 | 1.00 | 45.67 | C |
| ATOM | 7575 | N | SER | C | 47 | −8.341 | 22.384 | 32.062 | 1.00 | 46.17 | C |
| ATOM | 7576 | CA | SER | C | 47 | −9.421 | 22.337 | 33.049 | 1.00 | 43.29 | C |
| ATOM | 7577 | CB | SER | C | 47 | −10.747 | 22.081 | 32.340 | 1.00 | 60.97 | C |
| ATOM | 7578 | OG | SER | C | 47 | −10.710 | 20.833 | 31.664 | 1.00 | 62.18 | C |
| ATOM | 7579 | C | SER | C | 47 | −9.247 | 21.290 | 34.148 | 1.00 | 39.98 | C |
| ATOM | 7580 | O | SER | C | 47 | −8.516 | 20.314 | 33.966 | 1.00 | 37.12 | C |
| ATOM | 7581 | N | GLU | C | 48 | −9.921 | 21.513 | 35.284 | 1.00 | 28.83 | C |
| ATOM | 7582 | CA | GLU | C | 48 | −9.897 | 20.593 | 36.422 | 1.00 | 33.32 | C |
| ATOM | 7583 | CB | GLU | C | 48 | −10.882 | 21.042 | 37.502 | 1.00 | 162.61 | C |
| ATOM | 7584 | CG | GLU | C | 48 | −10.690 | 22.454 | 38.025 | 1.00 | 167.35 | C |
| ATOM | 7585 | CD | GLU | C | 48 | −9.618 | 22.551 | 39.094 | 1.00 | 171.70 | C |
| ATOM | 7586 | OE1 | GLU | C | 48 | −8.425 | 22.382 | 38.766 | 1.00 | 173.44 | C |
| ATOM | 7587 | OE2 | GLU | C | 48 | −9.971 | 22.792 | 40.269 | 1.00 | 169.62 | C |
| ATOM | 7588 | C | GLU | C | 48 | −10.373 | 19.239 | 35.878 | 1.00 | 31.91 | C |
| ATOM | 7589 | O | GLU | C | 48 | −11.146 | 19.191 | 34.916 | 1.00 | 37.70 | C |
| ATOM | 7590 | N | GLY | C | 49 | −9.907 | 18.141 | 36.467 | 1.00 | 35.47 | C |
| ATOM | 7591 | CA | GLY | C | 49 | −10.364 | 16.853 | 36.002 | 1.00 | 33.13 | C |
| ATOM | 7592 | C | GLY | C | 49 | −9.426 | 15.683 | 36.139 | 1.00 | 35.14 | C |
| ATOM | 7593 | O | GLY | C | 49 | −8.271 | 15.812 | 36.521 | 1.00 | 37.98 | C |
| ATOM | 7594 | N | LEU | C | 50 | −9.969 | 14.520 | 35.826 | 1.00 | 47.61 | C |
| ATOM | 7595 | CA | LEU | C | 50 | −9.248 | 13.262 | 35.854 | 1.00 | 48.81 | C |
| ATOM | 7596 | CB | LEU | C | 50 | −10.238 | 12.141 | 36.179 | 1.00 | 38.33 | C |
| ATOM | 7597 | CG | LEU | C | 50 | −10.142 | 11.365 | 37.493 | 1.00 | 39.46 | C |
| ATOM | 7598 | CD1 | LEU | C | 50 | −9.557 | 12.217 | 38.567 | 1.00 | 36.27 | C |
| ATOM | 7599 | CD2 | LEU | C | 50 | −11.509 | 10.870 | 37.889 | 1.00 | 39.85 | C |
| ATOM | 7600 | C | LEU | C | 50 | −8.671 | 13.077 | 34.445 | 1.00 | 45.19 | C |
| ATOM | 7601 | O | LEU | C | 50 | −9.383 | 13.213 | 33.455 | 1.00 | 43.91 | C |
| ATOM | 7602 | N | TYR | C | 51 | −7.382 | 12.780 | 34.351 | 1.00 | 38.45 | C |
| ATOM | 7603 | CA | TYR | C | 51 | −6.744 | 12.589 | 33.049 | 1.00 | 37.10 | C |
| ATOM | 7604 | CB | TYR | C | 51 | −5.774 | 13.735 | 32.738 | 1.00 | 33.61 | C |
| ATOM | 7605 | CG | TYR | C | 51 | −6.411 | 15.058 | 32.403 | 1.00 | 33.71 | C |
| ATOM | 7606 | CD1 | TYR | C | 51 | −7.007 | 15.839 | 33.381 | 1.00 | 34.57 | C |
| ATOM | 7607 | CE1 | TYR | C | 51 | −7.571 | 17.058 | 33.075 | 1.00 | 34.13 | C |
| ATOM | 7608 | CD2 | TYR | C | 51 | −6.400 | 15.531 | 31.101 | 1.00 | 35.27 | C |
| ATOM | 7609 | CE2 | TYR | C | 51 | −6.968 | 16.753 | 30.777 | 1.00 | 33.54 | C |
| ATOM | 7610 | CZ | TYR | C | 51 | −7.554 | 17.512 | 31.768 | 1.00 | 33.24 | C |
| ATOM | 7611 | OH | TYR | C | 51 | −8.138 | 18.718 | 31.437 | 1.00 | 33.47 | C |
| ATOM | 7612 | C | TYR | C | 51 | −5.948 | 11.301 | 32.949 | 1.00 | 37.28 | C |
| ATOM | 7613 | O | TYR | C | 51 | −5.324 | 10.851 | 33.920 | 1.00 | 38.17 | C |
| ATOM | 7614 | N | LEU | C | 52 | −5.972 | 10.706 | 31.763 | 1.00 | 39.66 | C |
| ATOM | 7615 | CA | LEU | C | 52 | −5.200 | 9.501 | 31.516 | 1.00 | 41.31 | C |
| ATOM | 7616 | CB | LEU | C | 52 | −5.935 | 8.552 | 30.571 | 1.00 | 40.80 | C |
| ATOM | 7617 | CG | LEU | C | 52 | −5.077 | 7.406 | 30.019 | 1.00 | 36.69 | C |
| ATOM | 7618 | CD1 | LEU | C | 52 | −4.382 | 6.662 | 31.143 | 1.00 | 38.72 | C |
| ATOM | 7619 | CD2 | LEU | C | 52 | −5.961 | 6.477 | 29.204 | 1.00 | 31.02 | C |
| ATOM | 7620 | C | LEU | C | 52 | −3.966 | 10.058 | 30.846 | 1.00 | 42.84 | C |
| ATOM | 7621 | O | LEU | C | 52 | −4.075 | 10.734 | 29.837 | 1.00 | 43.41 | C |
| ATOM | 7622 | N | ILE | C | 53 | −2.805 | 9.812 | 31.439 | 1.00 | 45.57 | C |
| ATOM | 7623 | CA | ILE | C | 53 | −1.536 | 10.295 | 30.917 | 1.00 | 42.17 | C |
| ATOM | 7624 | CB | ILE | C | 53 | −0.774 | 11.078 | 31.975 | 1.00 | 53.82 | C |
| ATOM | 7625 | CG2 | ILE | C | 53 | 0.494 | 11.639 | 31.383 | 1.00 | 54.46 | C |
| ATOM | 7626 | CG1 | ILE | C | 53 | −1.630 | 12.219 | 32.497 | 1.00 | 52.71 | C |
| ATOM | 7627 | CD1 | ILE | C | 53 | −0.948 | 13.036 | 33.575 | 1.00 | 48.53 | C |
| ATOM | 7628 | C | ILE | C | 53 | −0.679 | 9.110 | 30.526 | 1.00 | 42.23 | C |
| ATOM | 7629 | O | ILE | C | 53 | −0.598 | 8.125 | 31.263 | 1.00 | 45.31 | C |
| ATOM | 7630 | N | TYR | C | 54 | −0.024 | 9.192 | 29.377 | 1.00 | 44.54 | C |
| ATOM | 7631 | CA | TYR | C | 54 | 0.827 | 8.086 | 28.941 | 1.00 | 41.67 | C |
| ATOM | 7632 | CB | TYR | C | 54 | −0.016 | 7.014 | 28.233 | 1.00 | 34.38 | C |
| ATOM | 7633 | CG | TYR | C | 54 | −0.659 | 7.514 | 26.967 | 1.00 | 34.38 | C |
| ATOM | 7634 | CD1 | TYR | C | 54 | 0.002 | 7.430 | 25.755 | 1.00 | 34.38 | C |
| ATOM | 7635 | CE1 | TYR | C | 54 | −0.546 | 7.959 | 24.594 | 1.00 | 34.38 | C |
| ATOM | 7636 | CD2 | TYR | C | 54 | −1.903 | 8.140 | 26.991 | 1.00 | 34.38 | C |
| ATOM | 7637 | CE2 | TYR | C | 54 | −2.465 | 8.671 | 25.826 | 1.00 | 34.38 | C |
| ATOM | 7638 | CZ | TYR | C | 54 | −1.779 | 8.577 | 24.636 | 1.00 | 34.38 | C |
| ATOM | 7639 | OH | TYR | C | 54 | −2.327 | 9.115 | 23.492 | 1.00 | 34.38 | C |
| ATOM | 7640 | C | TYR | C | 54 | 1.967 | 8.541 | 28.025 | 1.00 | 40.26 | C |
| ATOM | 7641 | O | TYR | C | 54 | 1.951 | 9.631 | 27.452 | 1.00 | 39.11 | C |
| ATOM | 7642 | N | SER | C | 55 | 2.963 | 7.683 | 27.898 | 1.00 | 45.13 | C |
| ATOM | 7643 | CA | SER | C | 55 | 4.100 | 7.991 | 27.062 | 1.00 | 44.91 | C |
| ATOM | 7644 | CB | SER | C | 55 | 4.963 | 9.061 | 27.722 | 1.00 | 39.61 | C |
| ATOM | 7645 | OG | SER | C | 55 | 6.139 | 9.278 | 26.963 | 1.00 | 46.17 | C |
| ATOM | 7646 | C | SER | C | 55 | 4.955 | 6.761 | 26.797 | 1.00 | 47.89 | C |
| ATOM | 7647 | O | SER | C | 55 | 5.022 | 5.844 | 27.618 | 1.00 | 45.03 | C |
| ATOM | 7648 | N | GLN | C | 56 | 5.597 | 6.746 | 25.634 | 1.00 | 52.83 | C |
| ATOM | 7649 | CA | GLN | C | 56 | 6.481 | 5.656 | 25.284 | 1.00 | 53.53 | C |
| ATOM | 7650 | CB | GLN | C | 56 | 5.805 | 4.625 | 24.395 | 1.00 | 45.51 | C |
| ATOM | 7651 | CG | GLN | C | 56 | 6.722 | 3.439 | 24.176 | 1.00 | 47.11 | C |
| ATOM | 7652 | CD | GLN | C | 56 | 6.294 | 2.537 | 23.051 | 1.00 | 50.23 | C |
| ATOM | 7653 | OE1 | GLN | C | 56 | 6.548 | 2.821 | 21.881 | 1.00 | 49.76 | C |
| ATOM | 7654 | NE2 | GLN | C | 56 | 5.635 | 1.437 | 23.396 | 1.00 | 53.34 | C |
| ATOM | 7655 | C | GLN | C | 56 | 7.695 | 6.170 | 24.548 | 1.00 | 56.01 | C |
| ATOM | 7656 | O | GLN | C | 56 | 7.587 | 7.004 | 23.645 | 1.00 | 54.09 | C |
| ATOM | 7657 | N | VAL | C | 57 | 8.858 | 5.674 | 24.945 | 1.00 | 53.90 | C |
| ATOM | 7658 | CA | VAL | C | 57 | 10.095 | 6.050 | 24.287 | 1.00 | 48.27 | C |
| ATOM | 7659 | CB | VAL | C | 57 | 11.033 | 6.831 | 25.216 | 1.00 | 52.02 | C |
| ATOM | 7660 | CG1 | VAL | C | 57 | 10.338 | 8.079 | 25.704 | 1.00 | 53.22 | C |
| ATOM | 7661 | CG2 | VAL | C | 57 | 11.474 | 5.955 | 26.376 | 1.00 | 48.95 | C |
| ATOM | 7662 | C | VAL | C | 57 | 10.766 | 4.750 | 23.883 | 1.00 | 50.88 | C |
| ATOM | 7663 | O | VAL | C | 57 | 10.565 | 3.714 | 24.520 | 1.00 | 51.99 | C |
| ATOM | 7664 | N | LEU | C | 58 | 11.546 | 4.806 | 22.811 | 1.00 | 64.70 | C |
| ATOM | 7665 | CA | LEU | C | 58 | 12.250 | 3.633 | 22.333 | 1.00 | 63.15 | C |
| ATOM | 7666 | CB | LEU | C | 58 | 11.762 | 3.251 | 20.940 | 1.00 | 56.52 | C |
| ATOM | 7667 | CG | LEU | C | 58 | 11.880 | 1.783 | 20.545 | 1.00 | 58.54 | C |
| ATOM | 7668 | CD1 | LEU | C | 58 | 11.882 | 1.691 | 19.020 | 1.00 | 52.56 | C |
| ATOM | 7669 | CD2 | LEU | C | 58 | 13.144 | 1.171 | 21.124 | 1.00 | 56.32 | C |
| ATOM | 7670 | C | LEU | C | 58 | 13.730 | 3.978 | 22.270 | 1.00 | 66.12 | C |
| ATOM | 7671 | O | LEU | C | 58 | 14.138 | 4.856 | 21.509 | 1.00 | 67.75 | C |
| ATOM | 7672 | N | PHE | C | 59 | 14.529 | 3.298 | 23.085 | 1.00 | 60.02 | C |
| ATOM | 7673 | CA | PHE | C | 59 | 15.964 | 3.527 | 23.092 | 1.00 | 60.92 | C |
| ATOM | 7674 | CB | PHE | C | 59 | 16.525 | 3.391 | 24.503 | 1.00 | 59.88 | C |
| ATOM | 7675 | CG | PHE | C | 59 | 16.038 | 4.435 | 25.451 | 1.00 | 56.37 | C |
| ATOM | 7676 | CD1 | PHE | C | 59 | 15.072 | 4.130 | 26.405 | 1.00 | 54.81 | C |
| ATOM | 7677 | CD2 | PHE | C | 59 | 16.564 | 5.725 | 25.414 | 1.00 | 58.33 | C |
| ATOM | 7678 | CE1 | PHE | C | 59 | 14.639 | 5.095 | 27.315 | 1.00 | 60.73 | C |
| ATOM | 7679 | CE2 | PHE | C | 59 | 16.139 | 6.700 | 26.316 | 1.00 | 61.44 | C |
| ATOM | 7680 | CZ | PHE | C | 59 | 15.176 | 6.385 | 27.269 | 1.00 | 60.04 | C |
| ATOM | 7681 | C | PHE | C | 59 | 16.657 | 2.516 | 22.182 | 1.00 | 65.03 | C |
| ATOM | 7682 | O | PHE | C | 59 | 16.235 | 1.366 | 22.080 | 1.00 | 64.72 | C |
| ATOM | 7683 | N | LYS | C | 60 | 17.719 | 2.960 | 21.520 | 1.00 | 74.56 | C |
| ATOM | 7684 | CA | LYS | C | 60 | 18.488 | 2.105 | 20.628 | 1.00 | 75.91 | C |
| ATOM | 7685 | CB | LYS | C | 60 | 18.024 | 2.276 | 19.186 | 1.00 | 72.43 | C |
| ATOM | 7686 | CG | LYS | C | 60 | 18.852 | 1.499 | 18.182 | 1.00 | 78.62 | C |
| ATOM | 7687 | CD | LYS | C | 60 | 18.389 | 1.798 | 16.765 | 1.00 | 86.83 | C |
| ATOM | 7688 | CE | LYS | C | 60 | 19.213 | 1.047 | 15.730 | 1.00 | 89.58 | C |
| ATOM | 7689 | NZ | LYS | C | 60 | 18.704 | 1.303 | 14.354 | 1.00 | 89.92 | C |
| ATOM | 7690 | C | LYS | C | 60 | 19.956 | 2.474 | 20.728 | 1.00 | 75.42 | C |
| ATOM | 7691 | O | LYS | C | 60 | 20.312 | 3.653 | 20.762 | 1.00 | 78.17 | C |
| ATOM | 7692 | N | GLY | C | 61 | 20.808 | 1.459 | 20.778 | 1.00 | 87.81 | C |
| ATOM | 7693 | CA | GLY | C | 61 | 22.228 | 1.711 | 20.875 | 1.00 | 90.37 | C |
| ATOM | 7694 | C | GLY | C | 61 | 23.014 | 0.711 | 20.064 | 1.00 | 92.07 | C |
| ATOM | 7695 | O | GLY | C | 61 | 22.631 | −0.458 | 19.975 | 1.00 | 95.24 | C |
| ATOM | 7696 | N | GLN | C | 62 | 24.103 | 1.177 | 19.459 | 1.00 | 115.10 | C |
| ATOM | 7697 | CA | GLN | C | 62 | 24.965 | 0.315 | 18.660 | 1.00 | 115.41 | C |
| ATOM | 7698 | CB | GLN | C | 62 | 25.665 | 1.124 | 17.567 | 1.00 | 120.08 | C |
| ATOM | 7699 | CG | GLN | C | 62 | 24.723 | 1.867 | 16.631 | 1.00 | 124.21 | C |
| ATOM | 7700 | CD | GLN | C | 62 | 23.857 | 0.936 | 15.806 | 1.00 | 130.15 | C |
| ATOM | 7701 | OE1 | GLN | C | 62 | 23.089 | 0.141 | 16.344 | 1.00 | 129.75 | C |
| ATOM | 7702 | NE2 | GLN | C | 62 | 23.977 | 1.034 | 14.490 | 1.00 | 130.32 | C |
| ATOM | 7703 | C | GLN | C | 62 | 25.997 | −0.261 | 19.616 | 1.00 | 113.35 | C |
| ATOM | 7704 | O | GLN | C | 62 | 26.739 | 0.486 | 20.255 | 1.00 | 114.86 | C |
| ATOM | 7705 | N | GLY | C | 63 | 26.028 | −1.585 | 19.728 | 1.00 | 89.13 | C |
| ATOM | 7706 | CA | GLY | C | 63 | 26.977 | −2.219 | 20.625 | 1.00 | 87.12 | C |
| ATOM | 7707 | C | GLY | C | 63 | 26.857 | −1.659 | 22.029 | 1.00 | 85.20 | C |
| ATOM | 7708 | O | GLY | C | 63 | 25.911 | −0.936 | 22.325 | 1.00 | 83.24 | C |
| ATOM | 7709 | N | CYS | C | 64 | 27.821 | −1.968 | 22.890 | 1.00 | 118.85 | C |
| ATOM | 7710 | CA | CYS | C | 64 | 27.774 | −1.491 | 24.267 | 1.00 | 123.09 | C |
| ATOM | 7711 | C | CYS | C | 64 | 28.842 | −0.496 | 24.714 | 1.00 | 129.18 | C |
| ATOM | 7712 | O | CYS | C | 64 | 30.013 | −0.597 | 24.342 | 1.00 | 132.09 | C |
| ATOM | 7713 | CB | CYS | C | 64 | 27.762 | −2.681 | 25.223 | 1.00 | 173.51 | C |
| ATOM | 7714 | SG | CYS | C | 64 | 26.109 | −3.421 | 25.371 | 1.00 | 174.72 | C |
| ATOM | 7715 | N | PRO | C | 65 | 28.432 | 0.492 | 25.525 | 1.00 | 138.20 | C |
| ATOM | 7716 | CD | PRO | C | 65 | 27.027 | 0.862 | 25.768 | 1.00 | 163.63 | C |
| ATOM | 7717 | CA | PRO | C | 65 | 29.328 | 1.526 | 26.047 | 1.00 | 140.54 | C |
| ATOM | 7718 | CB | PRO | C | 65 | 28.363 | 2.569 | 26.615 | 1.00 | 166.16 | C |
| ATOM | 7719 | CG | PRO | C | 65 | 27.111 | 2.360 | 25.818 | 1.00 | 165.89 | C |
| ATOM | 7720 | C | PRO | C | 65 | 30.249 | 0.968 | 27.121 | 1.00 | 143.55 | C |
| ATOM | 7721 | O | PRO | C | 65 | 30.579 | −0.218 | 27.122 | 1.00 | 143.82 | C |
| ATOM | 7722 | N | SER | C | 66 | 30.650 | 1.838 | 28.039 | 1.00 | 139.54 | C |
| ATOM | 7723 | CA | SER | C | 66 | 31.527 | 1.453 | 29.130 | 1.00 | 143.14 | C |
| ATOM | 7724 | CB | SER | C | 66 | 32.541 | 2.567 | 29.397 | 1.00 | 148.32 | C |
| ATOM | 7725 | OG | SER | C | 66 | 33.394 | 2.238 | 30.478 | 1.00 | 145.48 | C |
| ATOM | 7726 | C | SER | C | 66 | 30.707 | 1.187 | 30.388 | 1.00 | 143.69 | C |
| ATOM | 7727 | O | SER | C | 66 | 29.487 | 1.363 | 30.396 | 1.00 | 137.11 | C |
| ATOM | 7728 | N | THR | C | 67 | 31.398 | 0.760 | 31.441 | 1.00 | 200.84 | C |
| ATOM | 7729 | CA | THR | C | 67 | 30.792 | 0.457 | 32.737 | 1.00 | 200.84 | C |
| ATOM | 7730 | CB | THR | C | 67 | 30.905 | 1.664 | 33.702 | 1.00 | 181.27 | C |
| ATOM | 7731 | OG1 | THR | C | 67 | 32.271 | 2.089 | 33.778 | 1.00 | 181.27 | C |
| ATOM | 7732 | CG2 | THR | C | 67 | 30.427 | 1.279 | 35.103 | 1.00 | 181.27 | C |
| ATOM | 7733 | C | THR | C | 67 | 29.326 | 0.038 | 32.662 | 1.00 | 200.84 | C |
| ATOM | 7734 | O | THR | C | 67 | 29.013 | −1.119 | 32.377 | 1.00 | 200.84 | C |
| ATOM | 7735 | N | HIS | C | 68 | 28.433 | 0.991 | 32.913 | 1.00 | 145.19 | C |
| ATOM | 7736 | CA | HIS | C | 68 | 27.005 | 0.714 | 32.902 | 1.00 | 140.31 | C |
| ATOM | 7737 | CB | HIS | C | 68 | 26.570 | 0.275 | 34.305 | 1.00 | 200.84 | C |
| ATOM | 7738 | CG | HIS | C | 68 | 25.303 | −0.521 | 34.329 | 1.00 | 200.84 | C |
| ATOM | 7739 | CD2 | HIS | C | 68 | 24.115 | −0.290 | 34.937 | 1.00 | 200.84 | C |
| ATOM | 7740 | ND1 | HIS | C | 68 | 25.173 | −1.730 | 33.681 | 1.00 | 200.84 | C |
| ATOM | 7741 | CE1 | HIS | C | 68 | 23.960 | −2.210 | 33.889 | 1.00 | 200.84 | C |
| ATOM | 7742 | NE2 | HIS | C | 68 | 23.298 | −1.355 | 34.648 | 1.00 | 200.84 | C |
| ATOM | 7743 | C | HIS | C | 68 | 26.223 | 1.954 | 32.475 | 1.00 | 136.14 | C |
| ATOM | 7744 | O | HIS | C | 68 | 26.584 | 3.078 | 32.821 | 1.00 | 135.59 | C |
| ATOM | 7745 | N | VAL | C | 69 | 25.155 | 1.742 | 31.715 | 1.00 | 86.75 | C |
| ATOM | 7746 | CA | VAL | C | 69 | 24.315 | 2.841 | 31.252 | 1.00 | 86.48 | C |
| ATOM | 7747 | CB | VAL | C | 69 | 24.293 | 2.924 | 29.708 | 1.00 | 87.23 | C |
| ATOM | 7748 | CG1 | VAL | C | 69 | 23.449 | 4.102 | 29.267 | 1.00 | 86.99 | C |
| ATOM | 7749 | CG2 | VAL | C | 69 | 25.703 | 3.062 | 29.167 | 1.00 | 85.49 | C |
| ATOM | 7750 | C | VAL | C | 69 | 22.884 | 2.649 | 31.750 | 1.00 | 86.07 | C |
| ATOM | 7751 | O | VAL | C | 69 | 22.331 | 1.552 | 31.678 | 1.00 | 89.63 | C |
| ATOM | 7752 | N | LEU | C | 70 | 22.293 | 3.719 | 32.267 | 1.00 | 81.07 | C |
| ATOM | 7753 | CA | LEU | C | 70 | 20.924 | 3.659 | 32.763 | 1.00 | 76.29 | C |
| ATOM | 7754 | CB | LEU | C | 70 | 20.839 | 4.142 | 34.213 | 1.00 | 76.29 | C |
| ATOM | 7755 | CG | LEU | C | 70 | 21.478 | 3.275 | 35.298 | 1.00 | 77.99 | C |
| ATOM | 7756 | CD1 | LEU | C | 70 | 21.188 | 3.886 | 36.657 | 1.00 | 73.03 | C |
| ATOM | 7757 | CD2 | LEU | C | 70 | 20.933 | 1.860 | 35.228 | 1.00 | 78.59 | C |
| ATOM | 7758 | C | LEU | C | 70 | 20.011 | 4.517 | 31.910 | 1.00 | 77.33 | C |
| ATOM | 7759 | O | LEU | C | 70 | 20.352 | 5.648 | 31.563 | 1.00 | 77.44 | C |
| ATOM | 7760 | N | LEU | C | 71 | 18.852 | 3.966 | 31.573 | 1.00 | 71.04 | C |
| ATOM | 7761 | CA | LEU | C | 71 | 17.867 | 4.679 | 30.772 | 1.00 | 70.98 | C |
| ATOM | 7762 | CB | LEU | C | 71 | 17.432 | 3.828 | 29.581 | 1.00 | 59.38 | C |
| ATOM | 7763 | CG | LEU | C | 71 | 18.545 | 3.410 | 28.625 | 1.00 | 57.05 | C |
| ATOM | 7764 | CD1 | LEU | C | 71 | 17.995 | 2.464 | 27.567 | 1.00 | 53.20 | C |
| ATOM | 7765 | CD2 | LEU | C | 71 | 19.139 | 4.643 | 27.986 | 1.00 | 57.17 | C |
| ATOM | 7766 | C | LEU | C | 71 | 16.669 | 4.961 | 31.660 | 1.00 | 75.01 | C |
| ATOM | 7767 | O | LEU | C | 71 | 16.147 | 4.055 | 32.308 | 1.00 | 74.50 | C |
| ATOM | 7768 | N | THR | C | 72 | 16.246 | 6.217 | 31.711 | 1.00 | 53.96 | C |
| ATOM | 7769 | CA | THR | C | 72 | 15.095 | 6.575 | 32.525 | 1.00 | 51.39 | C |
| ATOM | 7770 | CB | THR | C | 72 | 15.477 | 7.434 | 33.753 | 1.00 | 58.38 | C |
| ATOM | 7771 | OG1 | THR | C | 72 | 15.746 | 8.781 | 33.348 | 1.00 | 60.91 | C |
| ATOM | 7772 | CG2 | THR | C | 72 | 16.697 | 6.864 | 34.432 | 1.00 | 56.10 | C |
| ATOM | 7773 | C | THR | C | 72 | 14.077 | 7.351 | 31.714 | 1.00 | 54.02 | C |
| ATOM | 7774 | O | THR | C | 72 | 14.423 | 8.170 | 30.861 | 1.00 | 55.06 | C |
| ATOM | 7775 | N | HIS | C | 73 | 12.810 | 7.077 | 31.984 | 1.00 | 50.67 | C |
| ATOM | 7776 | CA | HIS | C | 73 | 11.711 | 7.750 | 31.312 | 1.00 | 51.12 | C |
| ATOM | 7777 | CB | HIS | C | 73 | 10.979 | 6.776 | 30.406 | 1.00 | 47.39 | C |
| ATOM | 7778 | CG | HIS | C | 73 | 9.900 | 7.410 | 29.595 | 1.00 | 50.26 | C |
| ATOM | 7779 | CD2 | HIS | C | 73 | 9.673 | 8.702 | 29.275 | 1.00 | 52.02 | C |
| ATOM | 7780 | ND1 | HIS | C | 73 | 8.879 | 6.684 | 29.022 | 1.00 | 54.87 | C |
| ATOM | 7781 | CE1 | HIS | C | 73 | 8.065 | 7.503 | 28.387 | 1.00 | 53.41 | C |
| ATOM | 7782 | NE2 | HIS | C | 73 | 8.525 | 8.733 | 28.525 | 1.00 | 53.58 | C |
| ATOM | 7783 | C | HIS | C | 73 | 10.808 | 8.175 | 32.445 | 1.00 | 50.20 | C |
| ATOM | 7784 | O | HIS | C | 73 | 10.535 | 7.383 | 33.346 | 1.00 | 46.06 | C |
| ATOM | 7785 | N | THR | C | 74 | 10.348 | 9.417 | 32.431 | 1.00 | 43.34 | C |
| ATOM | 7786 | CA | THR | C | 74 | 9.499 | 9.863 | 33.527 | 1.00 | 49.93 | C |
| ATOM | 7787 | CB | THR | C | 74 | 10.383 | 10.239 | 34.786 | 1.00 | 45.90 | C |
| ATOM | 7788 | OG1 | THR | C | 74 | 9.963 | 11.497 | 35.334 | 1.00 | 48.41 | C |
| ATOM | 7789 | CG2 | THR | C | 74 | 11.858 | 10.306 | 34.419 | 1.00 | 50.73 | C |
| ATOM | 7790 | C | THR | C | 74 | 8.562 | 11.009 | 33.162 | 1.00 | 48.78 | C |
| ATOM | 7791 | O | THR | C | 74 | 8.939 | 11.923 | 32.444 | 1.00 | 44.36 | C |
| ATOM | 7792 | N | ILE | C | 75 | 7.326 | 10.935 | 33.644 | 1.00 | 36.92 | C |
| ATOM | 7793 | CA | ILE | C | 75 | 6.346 | 11.983 | 33.386 | 1.00 | 38.22 | C |
| ATOM | 7794 | CB | ILE | C | 75 | 4.980 | 11.399 | 32.980 | 1.00 | 41.61 | C |
| ATOM | 7795 | CG2 | ILE | C | 75 | 3.945 | 12.506 | 32.807 | 1.00 | 41.10 | C |
| ATOM | 7796 | CG1 | ILE | C | 75 | 5.133 | 10.632 | 31.670 | 1.00 | 38.77 | C |
| ATOM | 7797 | CD1 | ILE | C | 75 | 3.838 | 10.068 | 31.152 | 1.00 | 42.06 | C |
| ATOM | 7798 | C | ILE | C | 75 | 6.185 | 12.783 | 34.668 | 1.00 | 41.36 | C |
| ATOM | 7799 | O | ILE | C | 75 | 5.850 | 12.229 | 35.716 | 1.00 | 38.13 | C |
| ATOM | 7800 | N | SER | C | 76 | 6.434 | 14.087 | 34.577 | 1.00 | 50.38 | C |
| ATOM | 7801 | CA | SER | C | 76 | 6.342 | 14.969 | 35.727 | 1.00 | 51.46 | C |
| ATOM | 7802 | CB | SER | C | 76 | 7.605 | 15.822 | 35.851 | 1.00 | 62.08 | C |
| ATOM | 7803 | OG | SER | C | 76 | 8.754 | 15.013 | 35.929 | 1.00 | 71.37 | C |
| ATOM | 7804 | C | SER | C | 76 | 5.167 | 15.908 | 35.627 | 1.00 | 47.46 | C |
| ATOM | 7805 | O | SER | C | 76 | 4.682 | 16.209 | 34.536 | 1.00 | 43.61 | C |
| ATOM | 7806 | N | ARG | C | 77 | 4.719 | 16.371 | 36.785 | 1.00 | 40.67 | C |
| ATOM | 7807 | CA | ARG | C | 77 | 3.645 | 17.334 | 36.850 | 1.00 | 45.85 | C |
| ATOM | 7808 | CB | ARG | C | 77 | 2.430 | 16.782 | 37.604 | 1.00 | 25.54 | C |
| ATOM | 7809 | CG | ARG | C | 77 | 1.536 | 17.898 | 38.159 | 1.00 | 27.72 | C |
| ATOM | 7810 | CD | ARG | C | 77 | 0.107 | 17.454 | 38.436 | 1.00 | 28.59 | C |
| ATOM | 7811 | NE | ARG | C | 77 | −0.223 | 17.347 | 39.852 | 1.00 | 31.03 | C |
| ATOM | 7812 | CZ | ARG | C | 77 | −1.234 | 17.987 | 40.437 | 1.00 | 34.46 | C |
| ATOM | 7813 | NH1 | ARG | C | 77 | −1.467 | 17.814 | 41.726 | 1.00 | 34.55 | C |
| ATOM | 7814 | NH2 | ARG | C | 77 | −2.000 | 18.813 | 39.750 | 1.00 | 34.32 | C |
| ATOM | 7815 | C | ARG | C | 77 | 4.188 | 18.537 | 37.594 | 1.00 | 44.19 | C |
| ATOM | 7816 | O | ARG | C | 77 | 4.850 | 18.396 | 38.615 | 1.00 | 49.94 | C |
| ATOM | 7817 | N | ILE | C | 78 | 3.949 | 19.720 | 37.057 | 1.00 | 43.36 | C |
| ATOM | 7818 | CA | ILE | C | 78 | 4.357 | 20.928 | 37.740 | 1.00 | 42.05 | C |
| ATOM | 7819 | CB | ILE | C | 78 | 5.437 | 21.773 | 36.925 | 1.00 | 48.24 | C |
| ATOM | 7820 | CG2 | ILE | C | 78 | 6.019 | 20.958 | 35.795 | 1.00 | 48.74 | C |
| ATOM | 7821 | CG1 | ILE | C | 78 | 4.851 | 23.067 | 36.401 | 1.00 | 49.91 | C |
| ATOM | 7822 | CD1 | ILE | C | 78 | 4.617 | 24.088 | 37.469 | 1.00 | 50.23 | C |
| ATOM | 7823 | C | ILE | C | 78 | 2.996 | 21.600 | 37.892 | 1.00 | 44.22 | C |
| ATOM | 7824 | O | ILE | C | 78 | 2.432 | 22.138 | 36.940 | 1.00 | 42.77 | C |
| ATOM | 7825 | N | ALA | C | 79 | 2.450 | 21.495 | 39.100 | 1.00 | 48.65 | C |
| ATOM | 7826 | CA | ALA | C | 79 | 1.134 | 22.035 | 39.410 | 1.00 | 45.25 | C |
| ATOM | 7827 | CB | ALA | C | 79 | 0.622 | 21.418 | 40.700 | 1.00 | 55.06 | C |
| ATOM | 7828 | C | ALA | C | 79 | 1.077 | 23.547 | 39.514 | 1.00 | 46.45 | C |
| ATOM | 7829 | O | ALA | C | 79 | 2.053 | 24.183 | 39.899 | 1.00 | 46.65 | C |
| ATOM | 7830 | N | VAL | C | 80 | −0.083 | 24.112 | 39.181 | 1.00 | 47.54 | C |
| ATOM | 7831 | CA | VAL | C | 80 | −0.274 | 25.552 | 39.251 | 1.00 | 50.03 | C |
| ATOM | 7832 | CB | VAL | C | 80 | −1.663 | 25.976 | 38.729 | 1.00 | 51.00 | C |
| ATOM | 7833 | CG1 | VAL | C | 80 | −1.832 | 27.471 | 38.856 | 1.00 | 49.40 | C |
| ATOM | 7834 | CG2 | VAL | C | 80 | −1.828 | 25.577 | 37.296 | 1.00 | 52.70 | C |
| ATOM | 7835 | C | VAL | C | 80 | −0.157 | 25.996 | 40.700 | 1.00 | 54.58 | C |
| ATOM | 7836 | O | VAL | C | 80 | 0.304 | 27.096 | 40.979 | 1.00 | 53.17 | C |
| ATOM | 7837 | N | SER | C | 81 | −0.566 | 25.137 | 41.624 | 1.00 | 68.88 | C |
| ATOM | 7838 | CA | SER | C | 81 | −0.502 | 25.482 | 43.034 | 1.00 | 71.74 | C |
| ATOM | 7839 | CB | SER | C | 81 | −1.706 | 24.899 | 43.763 | 1.00 | 63.03 | C |
| ATOM | 7840 | OG | SER | C | 81 | −2.906 | 25.485 | 43.290 | 1.00 | 66.07 | C |
| ATOM | 7841 | C | SER | C | 81 | 0.778 | 25.038 | 43.726 | 1.00 | 75.92 | C |
| ATOM | 7842 | O | SER | C | 81 | 0.875 | 25.136 | 44.947 | 1.00 | 76.44 | C |
| ATOM | 7843 | N | TYR | C | 82 | 1.755 | 24.554 | 42.957 | 1.00 | 45.06 | C |
| ATOM | 7844 | CA | TYR | C | 82 | 3.039 | 24.095 | 43.515 | 1.00 | 47.10 | C |
| ATOM | 7845 | CB | TYR | C | 82 | 2.859 | 22.780 | 44.277 | 1.00 | 37.01 | C |
| ATOM | 7846 | CG | TYR | C | 82 | 4.073 | 22.373 | 45.077 | 1.00 | 40.21 | C |
| ATOM | 7847 | CD1 | TYR | C | 82 | 4.716 | 21.157 | 44.839 | 1.00 | 42.50 | C |
| ATOM | 7848 | CE1 | TYR | C | 82 | 5.854 | 20.793 | 45.551 | 1.00 | 44.29 | C |
| ATOM | 7849 | CD2 | TYR | C | 82 | 4.600 | 23.216 | 46.056 | 1.00 | 42.30 | C |
| ATOM | 7850 | CE2 | TYR | C | 82 | 5.744 | 22.860 | 46.770 | 1.00 | 43.77 | C |
| ATOM | 7851 | CZ | TYR | C | 82 | 6.365 | 21.650 | 46.511 | 1.00 | 44.46 | C |
| ATOM | 7852 | OH | TYR | C | 82 | 7.513 | 21.311 | 47.194 | 1.00 | 42.88 | C |
| ATOM | 7853 | C | TYR | C | 82 | 4.048 | 23.892 | 42.389 | 1.00 | 46.53 | C |
| ATOM | 7854 | O | TYR | C | 82 | 4.373 | 22.760 | 42.032 | 1.00 | 46.71 | C |
| ATOM | 7855 | N | GLN | C | 83 | 4.533 | 25.005 | 41.841 | 1.00 | 62.90 | C |
| ATOM | 7856 | CA | GLN | C | 83 | 5.489 | 25.023 | 40.735 | 1.00 | 62.79 | C |
| ATOM | 7857 | CB | GLN | C | 83 | 5.895 | 26.460 | 40.409 | 1.00 | 74.11 | C |
| ATOM | 7858 | CG | GLN | C | 83 | 5.172 | 27.114 | 39.247 | 1.00 | 78.38 | C |
| ATOM | 7859 | CD | GLN | C | 83 | 3.791 | 27.572 | 39.614 | 1.00 | 79.42 | C |
| ATOM | 7860 | OE1 | GLN | C | 83 | 3.173 | 28.346 | 38.888 | 1.00 | 78.44 | C |
| ATOM | 7861 | NE2 | GLN | C | 83 | 3.291 | 27.098 | 40.752 | 1.00 | 76.92 | C |
| ATOM | 7862 | C | GLN | C | 83 | 6.770 | 24.224 | 40.915 | 1.00 | 60.97 | C |
| ATOM | 7863 | O | GLN | C | 83 | 7.836 | 24.803 | 41.074 | 1.00 | 58.42 | C |
| ATOM | 7864 | N | THR | C | 84 | 6.691 | 22.904 | 40.873 | 1.00 | 69.42 | C |
| ATOM | 7865 | CA | THR | C | 84 | 7.894 | 22.098 | 41.009 | 1.00 | 61.79 | C |
| ATOM | 7866 | CB | THR | C | 84 | 8.267 | 21.829 | 42.456 | 1.00 | 78.50 | C |
| ATOM | 7867 | OG1 | THR | C | 84 | 8.307 | 23.059 | 43.182 | 1.00 | 79.05 | C |
| ATOM | 7868 | CG2 | THR | C | 84 | 9.629 | 21.162 | 42.519 | 1.00 | 81.25 | C |
| ATOM | 7869 | C | THR | C | 84 | 7.629 | 20.765 | 40.390 | 1.00 | 58.30 | C |
| ATOM | 7870 | O | THR | C | 84 | 6.560 | 20.186 | 40.579 | 1.00 | 59.61 | C |
| ATOM | 7871 | N | LYS | C | 85 | 8.601 | 20.269 | 39.646 | 1.00 | 42.96 | C |
| ATOM | 7872 | CA | LYS | C | 85 | 8.447 | 18.980 | 39.023 | 1.00 | 42.82 | C |
| ATOM | 7873 | CB | LYS | C | 85 | 9.677 | 18.644 | 38.178 | 1.00 | 53.99 | C |
| ATOM | 7874 | CG | LYS | C | 85 | 9.599 | 19.027 | 36.717 | 1.00 | 50.93 | C |
| ATOM | 7875 | CD | LYS | C | 85 | 10.786 | 18.429 | 35.981 | 1.00 | 49.33 | C |
| ATOM | 7876 | CE | LYS | C | 85 | 10.688 | 18.628 | 34.472 | 1.00 | 47.63 | C |
| ATOM | 7877 | NZ | LYS | C | 85 | 10.660 | 20.056 | 34.077 | 1.00 | 45.88 | C |
| ATOM | 7878 | C | LYS | C | 85 | 8.293 | 17.917 | 40.103 | 1.00 | 42.62 | C |
| ATOM | 7879 | O | LYS | C | 85 | 9.132 | 17.821 | 40.996 | 1.00 | 43.07 | C |
| ATOM | 7880 | N | VAL | C | 86 | 7.205 | 17.151 | 40.041 | 1.00 | 44.57 | C |
| ATOM | 7881 | CA | VAL | C | 86 | 6.991 | 16.033 | 40.956 | 1.00 | 47.46 | C |
| ATOM | 7882 | CB | VAL | C | 86 | 5.824 | 16.262 | 41.965 | 1.00 | 37.55 | C |
| ATOM | 7883 | CG1 | VAL | C | 86 | 6.129 | 17.469 | 42.843 | 1.00 | 44.29 | C |
| ATOM | 7884 | CG2 | VAL | C | 86 | 4.528 | 16.475 | 41.246 | 1.00 | 38.82 | C |
| ATOM | 7885 | C | VAL | C | 86 | 6.680 | 14.878 | 40.017 | 1.00 | 48.13 | C |
| ATOM | 7886 | O | VAL | C | 86 | 5.847 | 14.996 | 39.121 | 1.00 | 52.82 | C |
| ATOM | 7887 | N | ASN | C | 87 | 7.382 | 13.772 | 40.189 | 1.00 | 59.45 | C |
| ATOM | 7888 | CA | ASN | C | 87 | 7.168 | 12.622 | 39.329 | 1.00 | 56.13 | C |
| ATOM | 7889 | CB | ASN | C | 87 | 8.312 | 11.632 | 39.522 | 1.00 | 61.20 | C |
| ATOM | 7890 | CG | ASN | C | 87 | 9.631 | 12.182 | 39.053 | 1.00 | 61.50 | C |
| ATOM | 7891 | OD1 | ASN | C | 87 | 9.692 | 13.249 | 38.438 | 1.00 | 64.11 | C |
| ATOM | 7892 | ND2 | ASN | C | 87 | 10.701 | 11.449 | 39.328 | 1.00 | 58.46 | C |
| ATOM | 7893 | C | ASN | C | 87 | 5.837 | 11.897 | 39.524 | 1.00 | 54.95 | C |
| ATOM | 7894 | O | ASN | C | 87 | 5.457 | 11.566 | 40.652 | 1.00 | 53.53 | C |
| ATOM | 7895 | N | LEU | C | 88 | 5.139 | 11.641 | 38.418 | 1.00 | 50.75 | C |
| ATOM | 7896 | CA | LEU | C | 88 | 3.866 | 10.922 | 38.453 | 1.00 | 49.74 | C |
| ATOM | 7897 | CB | LEU | C | 88 | 2.873 | 11.526 | 37.475 | 1.00 | 28.42 | C |
| ATOM | 7898 | CG | LEU | C | 88 | 2.468 | 12.964 | 37.728 | 1.00 | 25.35 | C |
| ATOM | 7899 | CD1 | LEU | C | 88 | 1.404 | 13.342 | 36.726 | 1.00 | 26.12 | C |
| ATOM | 7900 | CD2 | LEU | C | 88 | 1.971 | 13.119 | 39.181 | 1.00 | 23.92 | C |
| ATOM | 7901 | C | LEU | C | 88 | 4.091 | 9.483 | 38.054 | 1.00 | 48.98 | C |
| ATOM | 7902 | O | LEU | C | 88 | 3.568 | 8.570 | 38.667 | 1.00 | 47.45 | C |
| ATOM | 7903 | N | LEU | C | 89 | 4.869 | 9.297 | 37.004 | 1.00 | 47.06 | C |
| ATOM | 7904 | CA | LEU | C | 89 | 5.168 | 7.975 | 36.489 | 1.00 | 45.98 | C |
| ATOM | 7905 | CB | LEU | C | 89 | 4.316 | 7.708 | 35.254 | 1.00 | 45.63 | C |
| ATOM | 7906 | CG | LEU | C | 89 | 2.812 | 7.905 | 35.394 | 1.00 | 49.61 | C |
| ATOM | 7907 | CD1 | LEU | C | 89 | 2.207 | 8.050 | 34.026 | 1.00 | 49.34 | C |
| ATOM | 7908 | CD2 | LEU | C | 89 | 2.197 | 6.743 | 36.101 | 1.00 | 48.73 | C |
| ATOM | 7909 | C | LEU | C | 89 | 6.639 | 7.946 | 36.105 | 1.00 | 47.65 | C |
| ATOM | 7910 | O | LEU | C | 89 | 7.157 | 8.908 | 35.531 | 1.00 | 48.37 | C |
| ATOM | 7911 | N | SER | C | 90 | 7.310 | 6.845 | 36.404 | 1.00 | 39.39 | C |
| ATOM | 7912 | CA | SER | C | 90 | 8.723 | 6.734 | 36.096 | 1.00 | 38.73 | C |
| ATOM | 7913 | CB | SER | C | 90 | 9.534 | 7.499 | 37.137 | 1.00 | 58.48 | C |
| ATOM | 7914 | OG | SER | C | 90 | 10.919 | 7.337 | 36.909 | 1.00 | 62.70 | C |
| ATOM | 7915 | C | SER | C | 90 | 9.223 | 5.297 | 36.040 | 1.00 | 37.36 | C |
| ATOM | 7916 | O | SER | C | 90 | 8.785 | 4.438 | 36.811 | 1.00 | 37.18 | C |
| ATOM | 7917 | N | ALA | C | 91 | 10.147 | 5.034 | 35.123 | 1.00 | 38.93 | C |
| ATOM | 7918 | CA | ALA | C | 91 | 10.714 | 3.702 | 35.006 | 1.00 | 37.02 | C |
| ATOM | 7919 | CB | ALA | C | 91 | 9.952 | 2.892 | 33.963 | 1.00 | 34.90 | C |
| ATOM | 7920 | C | ALA | C | 91 | 12.193 | 3.791 | 34.651 | 1.00 | 40.99 | C |
| ATOM | 7921 | O | ALA | C | 91 | 12.664 | 4.812 | 34.152 | 1.00 | 42.86 | C |
| ATOM | 7922 | N | ILE | C | 92 | 12.920 | 2.718 | 34.928 | 1.00 | 48.45 | C |
| ATOM | 7923 | CA | ILE | C | 92 | 14.347 | 2.656 | 34.654 | 1.00 | 48.43 | C |
| ATOM | 7924 | CB | ILE | C | 92 | 15.157 | 2.697 | 35.961 | 1.00 | 46.43 | C |
| ATOM | 7925 | CG2 | ILE | C | 92 | 16.629 | 2.779 | 35.648 | 1.00 | 52.50 | C |
| ATOM | 7926 | CG1 | ILE | C | 92 | 14.742 | 3.909 | 36.793 | 1.00 | 44.54 | C |
| ATOM | 7927 | CD1 | ILE | C | 92 | 15.366 | 3.958 | 38.154 | 1.00 | 50.41 | C |
| ATOM | 7928 | C | ILE | C | 92 | 14.647 | 1.347 | 33.935 | 1.00 | 48.26 | C |
| ATOM | 7929 | O | ILE | C | 92 | 13.989 | 0.331 | 34.182 | 1.00 | 49.34 | C |
| ATOM | 7930 | N | LYS | C | 93 | 15.633 | 1.364 | 33.047 | 1.00 | 67.38 | C |
| ATOM | 7931 | CA | LYS | C | 93 | 15.987 | 0.159 | 32.317 | 1.00 | 68.03 | C |
| ATOM | 7932 | CB | LYS | C | 93 | 15.233 | 0.116 | 30.989 | 1.00 | 79.25 | C |
| ATOM | 7933 | CG | LYS | C | 93 | 14.928 | −1.288 | 30.501 | 1.00 | 82.94 | C |
| ATOM | 7934 | CD | LYS | C | 93 | 13.709 | −1.295 | 29.579 | 1.00 | 82.71 | C |
| ATOM | 7935 | CE | LYS | C | 93 | 13.288 | −2.717 | 29.208 | 1.00 | 82.61 | C |
| ATOM | 7936 | NZ | LYS | C | 93 | 12.068 | −2.757 | 28.335 | 1.00 | 85.18 | C |
| ATOM | 7937 | C | LYS | C | 93 | 17.487 | 0.136 | 32.077 | 1.00 | 69.73 | C |
| ATOM | 7938 | O | LYS | C | 93 | 18.075 | 1.158 | 31.711 | 1.00 | 67.65 | C |
| ATOM | 7939 | N | SER | C | 94 | 18.102 | −1.027 | 32.298 | 1.00 | 84.12 | C |
| ATOM | 7940 | CA | SER | C | 94 | 19.547 | −1.205 | 32.121 | 1.00 | 86.57 | C |
| ATOM | 7941 | CB | SER | C | 94 | 20.143 | −1.899 | 33.344 | 1.00 | 97.00 | C |
| ATOM | 7942 | OG | SER | C | 94 | 19.540 | −3.168 | 33.548 | 1.00 | 99.43 | C |
| ATOM | 7943 | C | SER | C | 94 | 19.830 | −2.041 | 30.877 | 1.00 | 89.16 | C |
| ATOM | 7944 | O | SER | C | 94 | 19.796 | −3.270 | 30.929 | 1.00 | 90.43 | C |
| ATOM | 7945 | N | PRO | C | 95 | 20.138 | −1.381 | 29.748 | 1.00 | 84.54 | C |
| ATOM | 7946 | CD | PRO | C | 95 | 20.425 | 0.062 | 29.641 | 1.00 | 88.99 | C |
| ATOM | 7947 | CA | PRO | C | 95 | 20.422 | −2.059 | 28.480 | 1.00 | 88.48 | C |
| ATOM | 7948 | CB | PRO | C | 95 | 20.548 | −0.900 | 27.499 | 1.00 | 91.93 | C |
| ATOM | 7949 | CG | PRO | C | 95 | 21.197 | 0.151 | 28.342 | 1.00 | 89.38 | C |
| ATOM | 7950 | C | PRO | C | 95 | 21.677 | −2.914 | 28.537 | 1.00 | 92.91 | C |
| ATOM | 7951 | O | PRO | C | 95 | 21.608 | −4.140 | 28.624 | 1.00 | 96.34 | C |
| ATOM | 7952 | N | CYS | C | 96 | 22.827 | −2.257 | 28.480 | 1.00 | 170.00 | C |
| ATOM | 7953 | CA | CYS | C | 96 | 24.094 | −2.956 | 28.530 | 1.00 | 169.79 | C |
| ATOM | 7954 | C | CYS | C | 96 | 24.437 | −3.312 | 29.962 | 1.00 | 170.89 | C |
| ATOM | 7955 | O | CYS | C | 96 | 24.779 | −2.446 | 30.767 | 1.00 | 171.19 | C |
| ATOM | 7956 | CB | CYS | C | 96 | 25.193 | −2.091 | 27.925 | 1.00 | 200.84 | C |
| ATOM | 7957 | SG | CYS | C | 96 | 25.017 | −1.886 | 26.127 | 1.00 | 200.84 | C |
| ATOM | 7958 | N | GLN | C | 97 | 24.329 | −4.599 | 30.270 | 1.00 | 200.84 | C |
| ATOM | 7959 | CA | GLN | C | 97 | 24.631 | −5.108 | 31.599 | 1.00 | 200.84 | C |
| ATOM | 7960 | CB | GLN | C | 97 | 23.999 | −6.491 | 31.774 | 1.00 | 161.99 | C |
| ATOM | 7961 | CG | GLN | C | 97 | 22.515 | −6.535 | 31.440 | 1.00 | 161.99 | C |
| ATOM | 7962 | CD | GLN | C | 97 | 22.006 | −7.950 | 31.212 | 1.00 | 161.99 | C |
| ATOM | 7963 | OE1 | GLN | C | 97 | 22.104 | −8.808 | 32.089 | 1.00 | 161.99 | C |
| ATOM | 7964 | NE2 | GLN | C | 97 | 21.459 | −8.197 | 30.026 | 1.00 | 161.99 | C |
| ATOM | 7965 | C | GLN | C | 97 | 26.149 | −5.202 | 31.751 | 1.00 | 200.84 | C |
| ATOM | 7966 | O | GLN | C | 97 | 26.669 | −6.199 | 32.254 | 1.00 | 200.84 | C |
| ATOM | 7967 | N | ARG | C | 98 | 26.846 | −4.158 | 31.301 | 1.00 | 200.84 | C |
| ATOM | 7968 | CA | ARG | C | 98 | 28.306 | −4.087 | 31.364 | 1.00 | 200.84 | C |
| ATOM | 7969 | CB | ARG | C | 98 | 28.787 | −4.429 | 32.784 | 1.00 | 190.81 | C |
| ATOM | 7970 | CG | ARG | C | 98 | 30.297 | −4.557 | 32.945 | 1.00 | 190.81 | C |
| ATOM | 7971 | CD | ARG | C | 98 | 31.028 | −3.249 | 32.669 | 1.00 | 190.81 | C |
| ATOM | 7972 | NE | ARG | C | 98 | 32.477 | −3.442 | 32.616 | 1.00 | 190.81 | C |
| ATOM | 7973 | CZ | ARG | C | 98 | 33.202 | −3.971 | 33.598 | 1.00 | 190.81 | C |
| ATOM | 7974 | NH1 | ARG | C | 98 | 34.513 | −4.107 | 33.454 | 1.00 | 190.81 | C |
| ATOM | 7975 | NH2 | ARG | C | 98 | 32.622 | −4.365 | 34.724 | 1.00 | 190.81 | C |
| ATOM | 7976 | C | ARG | C | 98 | 28.981 | −5.002 | 30.330 | 1.00 | 200.84 | C |
| ATOM | 7977 | O | ARG | C | 98 | 30.210 | −5.082 | 30.269 | 1.00 | 200.84 | C |
| ATOM | 7978 | N | GLU | C | 99 | 28.175 | −5.676 | 29.509 | 1.00 | 200.84 | C |
| ATOM | 7979 | CA | GLU | C | 99 | 28.692 | −6.579 | 28.478 | 1.00 | 200.84 | C |
| ATOM | 7980 | CB | GLU | C | 99 | 27.605 | −6.900 | 27.441 | 1.00 | 173.61 | C |
| ATOM | 7981 | CG | GLU | C | 99 | 26.322 | −6.094 | 27.587 | 1.00 | 173.61 | C |
| ATOM | 7982 | CD | GLU | C | 99 | 25.355 | −6.708 | 28.580 | 1.00 | 173.61 | C |
| ATOM | 7983 | OE1 | GLU | C | 99 | 25.808 | −7.140 | 29.662 | 1.00 | 173.61 | C |
| ATOM | 7984 | OE2 | GLU | C | 99 | 24.142 | −6.752 | 28.280 | 1.00 | 173.61 | C |
| ATOM | 7985 | C | GLU | C | 99 | 29.911 | −6.003 | 27.759 | 1.00 | 200.84 | C |
| ATOM | 7986 | O | GLU | C | 99 | 30.934 | −6.673 | 27.637 | 1.00 | 200.84 | C |
| ATOM | 7987 | N | THR | C | 100 | 29.790 | −4.762 | 27.289 | 1.00 | 198.64 | C |
| ATOM | 7988 | CA | THR | C | 100 | 30.865 | −4.070 | 26.575 | 1.00 | 198.64 | C |
| ATOM | 7989 | CB | THR | C | 100 | 31.980 | −3.594 | 27.544 | 1.00 | 165.95 | C |
| ATOM | 7990 | OG1 | THR | C | 100 | 32.477 | −4.707 | 28.294 | 1.00 | 166.23 | C |
| ATOM | 7991 | CG2 | THR | C | 100 | 31.443 | −2.544 | 28.507 | 1.00 | 167.12 | C |
| ATOM | 7992 | C | THR | C | 100 | 31.492 | −4.937 | 25.482 | 1.00 | 198.64 | C |
| ATOM | 7993 | O | THR | C | 100 | 32.106 | −5.965 | 25.762 | 1.00 | 198.64 | C |
| ATOM | 7994 | N | PRO | C | 101 | 31.349 | −4.522 | 24.214 | 1.00 | 197.90 | C |
| ATOM | 7995 | CD | PRO | C | 101 | 30.725 | −3.262 | 23.774 | 1.00 | 114.34 | C |
| ATOM | 7996 | CA | PRO | C | 101 | 31.896 | −5.253 | 23.068 | 1.00 | 197.90 | C |
| ATOM | 7997 | CB | PRO | C | 101 | 31.830 | −4.221 | 21.948 | 1.00 | 115.29 | C |
| ATOM | 7998 | CG | PRO | C | 101 | 30.583 | −3.476 | 22.280 | 1.00 | 113.32 | C |
| ATOM | 7999 | C | PRO | C | 101 | 33.311 | −5.778 | 23.296 | 1.00 | 197.90 | C |
| ATOM | 8000 | O | PRO | C | 101 | 34.266 | −5.006 | 23.391 | 1.00 | 197.90 | C |
| ATOM | 8001 | N | GLU | C | 102 | 33.428 | −7.099 | 23.382 | 1.00 | 200.84 | C |
| ATOM | 8002 | CA | GLU | C | 102 | 34.708 | −7.766 | 23.597 | 1.00 | 200.84 | C |
| ATOM | 8003 | CB | GLU | C | 102 | 34.541 | −9.272 | 23.355 | 1.00 | 133.92 | C |
| ATOM | 8004 | CG | GLU | C | 102 | 35.816 | −10.098 | 23.465 | 1.00 | 135.12 | C |
| ATOM | 8005 | CD | GLU | C | 102 | 36.744 | −9.914 | 22.278 | 1.00 | 136.43 | C |
| ATOM | 8006 | OE1 | GLU | C | 102 | 36.316 | −10.189 | 21.134 | 1.00 | 137.24 | C |
| ATOM | 8007 | OE2 | GLU | C | 102 | 37.902 | −9.493 | 22.490 | 1.00 | 135.55 | C |
| ATOM | 8008 | C | GLU | C | 102 | 35.809 | −7.200 | 22.698 | 1.00 | 199.36 | C |
| ATOM | 8009 | O | GLU | C | 102 | 36.954 | −7.042 | 23.127 | 1.00 | 196.42 | C |
| ATOM | 8010 | N | GLY | C | 103 | 35.456 | −6.893 | 21.454 | 1.00 | 139.43 | C |
| ATOM | 8011 | CA | GLY | C | 103 | 36.424 | −6.352 | 20.516 | 1.00 | 136.75 | C |
| ATOM | 8012 | C | GLY | C | 103 | 36.101 | −6.759 | 19.092 | 1.00 | 134.62 | C |
| ATOM | 8013 | O | GLY | C | 103 | 36.676 | −6.236 | 18.137 | 1.00 | 129.11 | C |
| ATOM | 8014 | N | ALA | C | 104 | 35.174 | −7.703 | 18.953 | 1.00 | 198.10 | C |
| ATOM | 8015 | CA | ALA | C | 104 | 34.755 | −8.192 | 17.646 | 1.00 | 198.10 | C |
| ATOM | 8016 | CB | ALA | C | 104 | 35.423 | −9.541 | 17.354 | 1.00 | 97.10 | C |
| ATOM | 8017 | C | ALA | C | 104 | 33.232 | −8.335 | 17.606 | 1.00 | 198.10 | C |
| ATOM | 8018 | O | ALA | C | 104 | 32.666 | −9.140 | 18.345 | 1.00 | 198.10 | C |
| ATOM | 8019 | N | GLU | C | 105 | 32.592 | −7.538 | 16.747 | 1.00 | 175.47 | C |
| ATOM | 8020 | CA | GLU | C | 105 | 31.135 | −7.523 | 16.552 | 1.00 | 175.47 | C |
| ATOM | 8021 | CB | GLU | C | 105 | 30.505 | −8.864 | 16.953 | 1.00 | 200.84 | C |
| ATOM | 8022 | CG | GLU | C | 105 | 30.883 | −10.029 | 16.054 | 1.00 | 200.84 | C |
| ATOM | 8023 | CD | GLU | C | 105 | 30.458 | −9.816 | 14.615 | 1.00 | 200.84 | C |
| ATOM | 8024 | OE1 | GLU | C | 105 | 30.973 | −8.877 | 13.972 | 1.00 | 200.84 | C |
| ATOM | 8025 | OE2 | GLU | C | 105 | 29.605 | −10.587 | 14.126 | 1.00 | 200.77 | C |
| ATOM | 8026 | C | GLU | C | 105 | 30.413 | −6.392 | 17.286 | 1.00 | 175.47 | C |
| ATOM | 8027 | O | GLU | C | 105 | 30.692 | −5.216 | 17.051 | 1.00 | 175.47 | C |
| ATOM | 8028 | N | ALA | C | 106 | 29.478 | −6.768 | 18.159 | 1.00 | 176.37 | C |
| ATOM | 8029 | CA | ALA | C | 106 | 28.672 | −5.836 | 18.951 | 1.00 | 176.37 | C |
| ATOM | 8030 | CB | ALA | C | 106 | 29.500 | −4.614 | 19.357 | 1.00 | 73.32 | C |
| ATOM | 8031 | C | ALA | C | 106 | 27.413 | −5.392 | 18.206 | 1.00 | 176.37 | C |
| ATOM | 8032 | O | ALA | C | 106 | 27.381 | −4.317 | 17.605 | 1.00 | 176.37 | C |
| ATOM | 8033 | N | LYS | C | 107 | 26.378 | −6.230 | 18.255 | 1.00 | 146.42 | C |
| ATOM | 8034 | CA | LYS | C | 107 | 25.100 | −5.945 | 17.599 | 1.00 | 140.54 | C |
| ATOM | 8035 | CB | LYS | C | 107 | 24.195 | −7.184 | 17.631 | 1.00 | 180.18 | C |
| ATOM | 8036 | CG | LYS | C | 107 | 24.557 | −8.294 | 16.651 | 1.00 | 180.13 | C |
| ATOM | 8037 | CD | LYS | C | 107 | 25.906 | −8.924 | 16.954 | 1.00 | 181.56 | C |
| ATOM | 8038 | CE | LYS | C | 107 | 26.131 | −10.155 | 16.088 | 1.00 | 177.60 | C |
| ATOM | 8039 | NZ | LYS | C | 107 | 25.976 | −9.856 | 14.638 | 1.00 | 177.86 | C |
| ATOM | 8040 | C | LYS | C | 107 | 24.365 | −4.781 | 18.265 | 1.00 | 136.02 | C |
| ATOM | 8041 | O | LYS | C | 107 | 24.743 | −4.332 | 19.348 | 1.00 | 135.15 | C |
| ATOM | 8042 | N | PRO | C | 108 | 23.301 | −4.274 | 17.616 | 1.00 | 102.95 | C |
| ATOM | 8043 | CD | PRO | C | 108 | 22.857 | −4.575 | 16.242 | 1.00 | 74.78 | C |
| ATOM | 8044 | CA | PRO | C | 108 | 22.528 | −3.161 | 18.174 | 1.00 | 101.69 | C |
| ATOM | 8045 | CB | PRO | C | 108 | 21.880 | −2.548 | 16.939 | 1.00 | 73.93 | C |
| ATOM | 8046 | CG | PRO | C | 108 | 21.578 | −3.754 | 16.115 | 1.00 | 71.31 | C |
| ATOM | 8047 | C | PRO | C | 108 | 21.496 | −3.668 | 19.176 | 1.00 | 100.92 | C |
| ATOM | 8048 | O | PRO | C | 108 | 21.133 | −4.845 | 19.154 | 1.00 | 99.10 | C |
| ATOM | 8049 | N | TRP | C | 109 | 21.035 | −2.784 | 20.057 | 1.00 | 86.14 | C |
| ATOM | 8050 | CA | TRP | C | 109 | 20.036 | −3.158 | 21.051 | 1.00 | 83.59 | C |
| ATOM | 8051 | CB | TRP | C | 109 | 20.654 | −3.261 | 22.448 | 1.00 | 81.46 | C |
| ATOM | 8052 | CG | TRP | C | 109 | 21.381 | −2.036 | 22.903 | 1.00 | 81.15 | C |
| ATOM | 8053 | CD2 | TRP | C | 109 | 20.808 | −0.865 | 23.498 | 1.00 | 80.16 | C |
| ATOM | 8054 | CE2 | TRP | C | 109 | 21.870 | 0.025 | 23.775 | 1.00 | 79.41 | C |
| ATOM | 8055 | CE3 | TRP | C | 109 | 19.501 | −0.481 | 23.823 | 1.00 | 80.26 | C |
| ATOM | 8056 | CD1 | TRP | C | 109 | 22.724 | −1.814 | 22.843 | 1.00 | 78.25 | C |
| ATOM | 8057 | NE1 | TRP | C | 109 | 23.028 | −0.579 | 23.368 | 1.00 | 76.57 | C |
| ATOM | 8058 | CZ2 | TRP | C | 109 | 21.666 | 1.275 | 24.360 | 1.00 | 75.86 | C |
| ATOM | 8059 | CZ3 | TRP | C | 109 | 19.299 | 0.762 | 24.406 | 1.00 | 80.02 | C |
| ATOM | 8060 | CH2 | TRP | C | 109 | 20.378 | 1.625 | 24.668 | 1.00 | 75.66 | C |
| ATOM | 8061 | C | TRP | C | 109 | 18.884 | −2.170 | 21.092 | 1.00 | 85.20 | C |
| ATOM | 8062 | O | TRP | C | 109 | 19.068 | −0.967 | 20.890 | 1.00 | 86.81 | C |
| ATOM | 8063 | N | TYR | C | 110 | 17.691 | −2.696 | 21.348 | 1.00 | 69.13 | C |
| ATOM | 8064 | CA | TYR | C | 110 | 16.480 | −1.888 | 21.438 | 1.00 | 67.77 | C |
| ATOM | 8065 | CB | TYR | C | 110 | 15.493 | −2.277 | 20.347 | 1.00 | 66.55 | C |
| ATOM | 8066 | CG | TYR | C | 110 | 15.981 | −2.030 | 18.936 | 1.00 | 70.10 | C |
| ATOM | 8067 | CD1 | TYR | C | 110 | 16.965 | −2.832 | 18.360 | 1.00 | 74.19 | C |
| ATOM | 8068 | CE1 | TYR | C | 110 | 17.381 | −2.625 | 17.046 | 1.00 | 74.51 | C |
| ATOM | 8069 | CD2 | TYR | C | 110 | 15.430 | −1.014 | 18.165 | 1.00 | 70.33 | C |
| ATOM | 8070 | CE2 | TYR | C | 110 | 15.838 | −0.799 | 16.858 | 1.00 | 75.70 | C |
| ATOM | 8071 | CZ | TYR | C | 110 | 16.809 | −1.603 | 16.303 | 1.00 | 75.99 | C |
| ATOM | 8072 | OH | TYR | C | 110 | 17.195 | −1.372 | 15.002 | 1.00 | 77.19 | C |
| ATOM | 8073 | C | TYR | C | 110 | 15.832 | −2.109 | 22.796 | 1.00 | 66.28 | C |
| ATOM | 8074 | O | TYR | C | 110 | 15.739 | −3.242 | 23.270 | 1.00 | 67.47 | C |
| ATOM | 8075 | N | GLU | C | 111 | 15.383 | −1.023 | 23.415 | 1.00 | 57.34 | C |
| ATOM | 8076 | CA | GLU | C | 111 | 14.757 | −1.088 | 24.732 | 1.00 | 55.36 | C |
| ATOM | 8077 | CB | GLU | C | 111 | 15.800 | −0.814 | 25.806 | 1.00 | 80.70 | C |
| ATOM | 8078 | CG | GLU | C | 111 | 15.661 | −1.696 | 27.006 | 1.00 | 87.62 | C |
| ATOM | 8079 | CD | GLU | C | 111 | 15.827 | −3.159 | 26.656 | 1.00 | 92.59 | C |
| ATOM | 8080 | OE1 | GLU | C | 111 | 16.899 | −3.515 | 26.127 | 1.00 | 92.80 | C |
| ATOM | 8081 | OE2 | GLU | C | 111 | 14.892 | −3.954 | 26.904 | 1.00 | 99.75 | C |
| ATOM | 8082 | C | GLU | C | 111 | 13.623 | −0.066 | 24.846 | 1.00 | 55.94 | C |
| ATOM | 8083 | O | GLU | C | 111 | 13.867 | 1.136 | 24.925 | 1.00 | 61.30 | C |
| ATOM | 8084 | N | PRO | C | 112 | 12.363 | −0.530 | 24.835 | 1.00 | 50.88 | C |
| ATOM | 8085 | CD | PRO | C | 112 | 11.917 | −1.879 | 24.430 | 1.00 | 46.13 | C |
| ATOM | 8086 | CA | PRO | C | 112 | 11.217 | 0.374 | 24.941 | 1.00 | 50.11 | C |
| ATOM | 8087 | CB | PRO | C | 112 | 10.171 | −0.324 | 24.084 | 1.00 | 42.20 | C |
| ATOM | 8088 | CG | PRO | C | 112 | 10.397 | −1.759 | 24.441 | 1.00 | 43.33 | C |
| ATOM | 8089 | C | PRO | C | 112 | 10.754 | 0.524 | 26.390 | 1.00 | 47.14 | C |
| ATOM | 8090 | O | PRO | C | 112 | 11.017 | −0.342 | 27.227 | 1.00 | 47.27 | C |
| ATOM | 8091 | N | ILE | C | 113 | 10.071 | 1.626 | 26.682 | 1.00 | 43.89 | C |
| ATOM | 8092 | CA | ILE | C | 113 | 9.531 | 1.856 | 28.018 | 1.00 | 45.86 | C |
| ATOM | 8093 | CB | ILE | C | 113 | 10.419 | 2.782 | 28.856 | 1.00 | 60.80 | C |
| ATOM | 8094 | CG2 | ILE | C | 113 | 9.769 | 3.017 | 30.211 | 1.00 | 56.82 | C |
| ATOM | 8095 | CG1 | ILE | C | 113 | 11.798 | 2.166 | 29.052 | 1.00 | 57.86 | C |
| ATOM | 8096 | CD1 | ILE | C | 113 | 12.681 | 2.976 | 29.994 | 1.00 | 58.34 | C |
| ATOM | 8097 | C | ILE | C | 113 | 8.163 | 2.522 | 27.909 | 1.00 | 50.08 | C |
| ATOM | 8098 | O | ILE | C | 113 | 8.020 | 3.541 | 27.241 | 1.00 | 53.48 | C |
| ATOM | 8099 | N | TYR | C | 114 | 7.162 | 1.951 | 28.566 | 1.00 | 38.91 | C |
| ATOM | 8100 | CA | TYR | C | 114 | 5.829 | 2.532 | 28.526 | 1.00 | 37.19 | C |
| ATOM | 8101 | CB | TYR | C | 114 | 4.799 | 1.506 | 28.051 | 1.00 | 38.06 | C |
| ATOM | 8102 | CG | TYR | C | 114 | 3.431 | 2.089 | 27.796 | 1.00 | 35.92 | C |
| ATOM | 8103 | CD1 | TYR | C | 114 | 3.125 | 2.679 | 26.575 | 1.00 | 40.21 | C |
| ATOM | 8104 | CE1 | TYR | C | 114 | 1.859 | 3.239 | 26.338 | 1.00 | 39.78 | C |
| ATOM | 8105 | CD2 | TYR | C | 114 | 2.442 | 2.068 | 28.789 | 1.00 | 36.09 | C |
| ATOM | 8106 | CE2 | TYR | C | 114 | 1.177 | 2.622 | 28.567 | 1.00 | 40.42 | C |
| ATOM | 8107 | CZ | TYR | C | 114 | 0.892 | 3.207 | 27.338 | 1.00 | 40.50 | C |
| ATOM | 8108 | OH | TYR | C | 114 | −0.354 | 3.762 | 27.119 | 1.00 | 40.00 | C |
| ATOM | 8109 | C | TYR | C | 114 | 5.456 | 3.031 | 29.917 | 1.00 | 37.03 | C |
| ATOM | 8110 | O | TYR | C | 114 | 5.813 | 2.420 | 30.920 | 1.00 | 39.94 | C |
| ATOM | 8111 | N | LEU | C | 115 | 4.726 | 4.141 | 29.959 | 1.00 | 46.66 | C |
| ATOM | 8112 | CA | LEU | C | 115 | 4.295 | 4.756 | 31.205 | 1.00 | 45.70 | C |
| ATOM | 8113 | CB | LEU | C | 115 | 5.181 | 5.960 | 31.496 | 1.00 | 56.80 | C |
| ATOM | 8114 | CG | LEU | C | 115 | 6.191 | 5.936 | 32.645 | 1.00 | 60.05 | C |
| ATOM | 8115 | CD1 | LEU | C | 115 | 6.856 | 4.583 | 32.782 | 1.00 | 58.43 | C |
| ATOM | 8116 | CD2 | LEU | C | 115 | 7.215 | 7.025 | 32.393 | 1.00 | 63.35 | C |
| ATOM | 8117 | C | LEU | C | 115 | 2.849 | 5.214 | 31.058 | 1.00 | 48.14 | C |
| ATOM | 8118 | O | LEU | C | 115 | 2.470 | 5.744 | 30.010 | 1.00 | 50.67 | C |
| ATOM | 8119 | N | GLY | C | 116 | 2.046 | 5.023 | 32.102 | 1.00 | 38.12 | C |
| ATOM | 8120 | CA | GLY | C | 116 | 0.660 | 5.449 | 32.024 | 1.00 | 32.46 | C |
| ATOM | 8121 | C | GLY | C | 116 | −0.140 | 5.278 | 33.296 | 1.00 | 36.31 | C |
| ATOM | 8122 | O | GLY | C | 116 | 0.005 | 4.286 | 34.016 | 1.00 | 35.19 | C |
| ATOM | 8123 | N | GLY | C | 117 | −1.000 | 6.253 | 33.563 | 1.00 | 41.02 | C |
| ATOM | 8124 | CA | GLY | C | 117 | −1.833 | 6.209 | 34.751 | 1.00 | 44.02 | C |
| ATOM | 8125 | C | GLY | C | 117 | −2.851 | 7.330 | 34.767 | 1.00 | 45.33 | C |
| ATOM | 8126 | O | GLY | C | 117 | −2.776 | 8.239 | 33.948 | 1.00 | 46.71 | C |
| ATOM | 8127 | N | VAL | C | 118 | −3.803 | 7.271 | 35.694 | 1.00 | 29.05 | C |
| ATOM | 8128 | CA | VAL | C | 118 | −4.831 | 8.304 | 35.786 | 1.00 | 31.83 | C |
| ATOM | 8129 | CB | VAL | C | 118 | −6.220 | 7.657 | 35.995 | 1.00 | 31.59 | C |
| ATOM | 8130 | CG1 | VAL | C | 118 | −7.291 | 8.727 | 36.165 | 1.00 | 32.99 | C |
| ATOM | 8131 | CG2 | VAL | C | 118 | −6.539 | 6.771 | 34.806 | 1.00 | 31.65 | C |
| ATOM | 8132 | C | VAL | C | 118 | −4.512 | 9.277 | 36.921 | 1.00 | 29.74 | C |
| ATOM | 8133 | O | VAL | C | 118 | −4.100 | 8.852 | 38.000 | 1.00 | 33.69 | C |
| ATOM | 8134 | N | PHE | C | 119 | −4.706 | 10.573 | 36.670 | 1.00 | 32.16 | C |
| ATOM | 8135 | CA | PHE | C | 119 | −4.395 | 11.606 | 37.660 | 1.00 | 33.47 | C |
| ATOM | 8136 | CB | PHE | C | 119 | −2.988 | 12.173 | 37.408 | 1.00 | 27.78 | C |
| ATOM | 8137 | CG | PHE | C | 119 | −1.885 | 11.152 | 37.536 | 1.00 | 25.38 | C |
| ATOM | 8138 | CD1 | PHE | C | 119 | −1.554 | 10.326 | 36.469 | 1.00 | 23.57 | C |
| ATOM | 8139 | CD2 | PHE | C | 119 | −1.213 | 10.970 | 38.747 | 1.00 | 24.06 | C |
| ATOM | 8140 | CE1 | PHE | C | 119 | −0.570 | 9.328 | 36.603 | 1.00 | 24.64 | C |
| ATOM | 8141 | CE2 | PHE | C | 119 | −0.240 | 9.985 | 38.886 | 1.00 | 24.87 | C |
| ATOM | 8142 | CZ | PHE | C | 119 | 0.083 | 9.159 | 37.806 | 1.00 | 23.96 | C |
| ATOM | 8143 | C | PHE | C | 119 | −5.378 | 12.780 | 37.727 | 1.00 | 34.38 | C |
| ATOM | 8144 | O | PHE | C | 119 | −6.014 | 13.137 | 36.727 | 1.00 | 30.48 | C |
| ATOM | 8145 | N | GLN | C | 120 | −5.488 | 13.370 | 38.922 | 1.00 | 38.70 | C |
| ATOM | 8146 | CA | GLN | C | 120 | −6.344 | 14.525 | 39.171 | 1.00 | 42.07 | C |
| ATOM | 8147 | CB | GLN | C | 120 | −6.725 | 14.594 | 40.647 | 1.00 | 69.48 | C |
| ATOM | 8148 | CG | GLN | C | 120 | −8.211 | 14.588 | 40.943 | 1.00 | 69.63 | C |
| ATOM | 8149 | CD | GLN | C | 120 | −8.971 | 15.649 | 40.180 | 1.00 | 69.23 | C |
| ATOM | 8150 | OE1 | GLN | C | 120 | −8.506 | 16.773 | 40.040 | 1.00 | 68.79 | C |
| ATOM | 8151 | NE2 | GLN | C | 120 | −10.155 | 15.300 | 39.694 | 1.00 | 72.06 | C |
| ATOM | 8152 | C | GLN | C | 120 | −5.459 | 15.723 | 38.830 | 1.00 | 41.21 | C |
| ATOM | 8153 | O | GLN | C | 120 | −4.389 | 15.883 | 39.416 | 1.00 | 43.91 | C |
| ATOM | 8154 | N | LEU | C | 121 | −5.900 | 16.562 | 37.894 | 1.00 | 32.45 | C |
| ATOM | 8155 | CA | LEU | C | 121 | −5.123 | 17.727 | 37.465 | 1.00 | 34.11 | C |
| ATOM | 8156 | CB | LEU | C | 121 | −4.678 | 17.545 | 36.009 | 1.00 | 30.55 | C |
| ATOM | 8157 | CG | LEU | C | 121 | −3.322 | 16.900 | 35.711 | 1.00 | 28.82 | C |
| ATOM | 8158 | CD1 | LEU | C | 121 | −2.912 | 15.933 | 36.781 | 1.00 | 30.21 | C |
| ATOM | 8159 | CD2 | LEU | C | 121 | −3.405 | 16.218 | 34.363 | 1.00 | 24.31 | C |
| ATOM | 8160 | C | LEU | C | 121 | −5.902 | 19.016 | 37.582 | 1.00 | 36.15 | C |
| ATOM | 8161 | O | LEU | C | 121 | −7.125 | 19.022 | 37.444 | 1.00 | 41.42 | C |
| ATOM | 8162 | N | GLU | C | 122 | −5.175 | 20.106 | 37.826 | 1.00 | 44.38 | C |
| ATOM | 8163 | CA | GLU | C | 122 | −5.748 | 21.451 | 37.959 | 1.00 | 45.92 | C |
| ATOM | 8164 | CB | GLU | C | 122 | −4.976 | 22.297 | 38.982 | 1.00 | 113.28 | C |
| ATOM | 8165 | CG | GLU | C | 122 | −4.902 | 21.809 | 40.410 | 1.00 | 116.32 | C |
| ATOM | 8166 | CD | GLU | C | 122 | −4.003 | 22.710 | 41.259 | 1.00 | 121.60 | C |
| ATOM | 8167 | OE1 | GLU | C | 122 | −2.790 | 22.803 | 40.957 | 1.00 | 123.10 | C |
| ATOM | 8168 | OE2 | GLU | C | 122 | −4.508 | 23.334 | 42.219 | 1.00 | 121.69 | C |
| ATOM | 8169 | C | GLU | C | 122 | −5.623 | 22.185 | 36.623 | 1.00 | 44.72 | C |
| ATOM | 8170 | O | GLU | C | 122 | −4.712 | 21.918 | 35.844 | 1.00 | 45.91 | C |
| ATOM | 8171 | N | LYS | C | 123 | −6.525 | 23.125 | 36.368 | 1.00 | 46.18 | C |
| ATOM | 8172 | CA | LYS | C | 123 | −6.434 | 23.922 | 35.156 | 1.00 | 47.03 | C |
| ATOM | 8173 | CB | LYS | C | 123 | −7.579 | 24.927 | 35.091 | 1.00 | 73.83 | C |
| ATOM | 8174 | CG | LYS | C | 123 | −7.419 | 25.923 | 33.975 | 1.00 | 69.99 | C |
| ATOM | 8175 | CD | LYS | C | 123 | −8.509 | 26.959 | 34.007 | 1.00 | 67.66 | C |
| ATOM | 8176 | CE | LYS | C | 123 | −8.291 | 27.991 | 32.912 | 1.00 | 69.39 | C |
| ATOM | 8177 | NZ | LYS | C | 123 | −9.309 | 29.089 | 32.969 | 1.00 | 73.40 | C |
| ATOM | 8178 | C | LYS | C | 123 | −5.096 | 24.676 | 35.237 | 1.00 | 47.85 | C |
| ATOM | 8179 | O | LYS | C | 123 | −4.811 | 25.359 | 36.228 | 1.00 | 52.61 | C |
| ATOM | 8180 | N | GLY | C | 124 | −4.269 | 24.539 | 34.207 | 1.00 | 38.64 | C |
| ATOM | 8181 | CA | GLY | C | 124 | −2.986 | 25.214 | 34.209 | 1.00 | 39.75 | C |
| ATOM | 8182 | C | GLY | C | 124 | −1.784 | 24.322 | 34.473 | 1.00 | 41.16 | C |
| ATOM | 8183 | O | GLY | C | 124 | −0.652 | 24.765 | 34.284 | 1.00 | 41.96 | C |
| ATOM | 8184 | N | ASP | C | 125 | −2.004 | 23.083 | 34.912 | 1.00 | 41.18 | C |
| ATOM | 8185 | CA | ASP | C | 125 | −0.888 | 22.178 | 35.189 | 1.00 | 39.97 | C |
| ATOM | 8186 | CB | ASP | C | 125 | −1.386 | 20.858 | 35.778 | 1.00 | 68.88 | C |
| ATOM | 8187 | CG | ASP | C | 125 | −1.909 | 21.004 | 37.199 | 1.00 | 74.34 | C |
| ATOM | 8188 | OD1 | ASP | C | 125 | −1.701 | 22.070 | 37.820 | 1.00 | 75.99 | C |
| ATOM | 8189 | OD2 | ASP | C | 125 | −2.522 | 20.037 | 37.697 | 1.00 | 73.00 | C |
| ATOM | 8190 | C | ASP | C | 125 | −0.062 | 21.894 | 33.941 | 1.00 | 39.02 | C |
| ATOM | 8191 | O | ASP | C | 125 | −0.577 | 21.893 | 32.821 | 1.00 | 35.93 | C |
| ATOM | 8192 | N | ARG | C | 126 | 1.233 | 21.670 | 34.141 | 1.00 | 56.42 | C |
| ATOM | 8193 | CA | ARG | C | 126 | 2.151 | 21.389 | 33.044 | 1.00 | 51.90 | C |
| ATOM | 8194 | CB | ARG | C | 126 | 3.328 | 22.368 | 33.075 | 1.00 | 82.47 | C |
| ATOM | 8195 | CG | ARG | C | 126 | 3.001 | 23.799 | 32.737 | 1.00 | 80.96 | C |
| ATOM | 8196 | CD | ARG | C | 126 | 4.207 | 24.694 | 33.025 | 1.00 | 85.88 | C |
| ATOM | 8197 | NE | ARG | C | 126 | 4.077 | 26.004 | 32.384 | 1.00 | 89.43 | C |
| ATOM | 8198 | CZ | ARG | C | 126 | 4.311 | 26.241 | 31.090 | 1.00 | 92.66 | C |
| ATOM | 8199 | NH1 | ARG | C | 126 | 4.153 | 27.466 | 30.588 | 1.00 | 92.33 | C |
| ATOM | 8200 | NH2 | ARG | C | 126 | 4.725 | 25.258 | 30.293 | 1.00 | 90.85 | C |
| ATOM | 8201 | C | ARG | C | 126 | 2.684 | 19.965 | 33.168 | 1.00 | 52.74 | C |
| ATOM | 8202 | O | ARG | C | 126 | 3.229 | 19.593 | 34.193 | 1.00 | 49.96 | C |
| ATOM | 8203 | N | LEU | C | 127 | 2.537 | 19.168 | 32.124 | 1.00 | 42.78 | C |
| ATOM | 8204 | CA | LEU | C | 127 | 3.028 | 17.794 | 32.162 | 1.00 | 39.16 | C |
| ATOM | 8205 | CB | LEU | C | 127 | 1.933 | 16.829 | 31.708 | 1.00 | 38.37 | C |
| ATOM | 8206 | CG | LEU | C | 127 | 0.679 | 16.907 | 32.581 | 1.00 | 37.35 | C |
| ATOM | 8207 | CD1 | LEU | C | 127 | −0.468 | 16.195 | 31.914 | 1.00 | 37.93 | C |
| ATOM | 8208 | CD2 | LEU | C | 127 | 0.970 | 16.298 | 33.923 | 1.00 | 38.01 | C |
| ATOM | 8209 | C | LEU | C | 127 | 4.250 | 17.659 | 31.263 | 1.00 | 38.91 | C |
| ATOM | 8210 | O | LEU | C | 127 | 4.266 | 18.184 | 30.152 | 1.00 | 35.11 | C |
| ATOM | 8211 | N | SER | C | 128 | 5.268 | 16.955 | 31.757 | 1.00 | 42.40 | C |
| ATOM | 8212 | CA | SER | C | 128 | 6.507 | 16.749 | 31.017 | 1.00 | 42.07 | C |
| ATOM | 8213 | CB | SER | C | 128 | 7.678 | 17.398 | 31.754 | 1.00 | 79.99 | C |
| ATOM | 8214 | OG | SER | C | 128 | 7.308 | 18.647 | 32.307 | 1.00 | 85.84 | C |
| ATOM | 8215 | C | SER | C | 128 | 6.806 | 15.260 | 30.860 | 1.00 | 41.25 | C |
| ATOM | 8216 | O | SER | C | 128 | 6.596 | 14.468 | 31.779 | 1.00 | 42.53 | C |
| ATOM | 8217 | N | ALA | C | 129 | 7.295 | 14.889 | 29.688 | 1.00 | 49.88 | C |
| ATOM | 8218 | CA | ALA | C | 129 | 7.662 | 13.514 | 29.414 | 1.00 | 50.85 | C |
| ATOM | 8219 | CB | ALA | C | 129 | 6.806 | 12.960 | 28.289 | 1.00 | 64.91 | C |
| ATOM | 8220 | C | ALA | C | 129 | 9.129 | 13.594 | 28.995 | 1.00 | 49.62 | C |
| ATOM | 8221 | O | ALA | C | 129 | 9.434 | 13.894 | 27.845 | 1.00 | 50.50 | C |
| ATOM | 8222 | N | GLU | C | 130 | 10.033 | 13.328 | 29.930 | 1.00 | 52.71 | C |
| ATOM | 8223 | CA | GLU | C | 130 | 11.454 | 13.423 | 29.657 | 1.00 | 53.68 | C |
| ATOM | 8224 | CB | GLU | C | 130 | 12.051 | 14.481 | 30.582 | 1.00 | 97.40 | C |
| ATOM | 8225 | CG | GLU | C | 130 | 11.411 | 15.845 | 30.404 | 1.00 | 96.94 | C |
| ATOM | 8226 | CD | GLU | C | 130 | 11.604 | 16.763 | 31.600 | 1.00 | 102.43 | C |
| ATOM | 8227 | OE1 | GLU | C | 130 | 11.171 | 17.936 | 31.523 | 1.00 | 103.80 | C |
| ATOM | 8228 | OE2 | GLU | C | 130 | 12.179 | 16.318 | 32.618 | 1.00 | 102.24 | C |
| ATOM | 8229 | C | GLU | C | 130 | 12.237 | 12.121 | 29.780 | 1.00 | 50.07 | C |
| ATOM | 8230 | O | GLU | C | 130 | 11.764 | 11.152 | 30.364 | 1.00 | 49.99 | C |
| ATOM | 8231 | N | ILE | C | 131 | 13.433 | 12.108 | 29.194 | 1.00 | 36.29 | C |
| ATOM | 8232 | CA | ILE | C | 131 | 14.346 | 10.969 | 29.250 | 1.00 | 38.83 | C |
| ATOM | 8233 | CB | ILE | C | 131 | 14.458 | 10.249 | 27.901 | 1.00 | 54.50 | C |
| ATOM | 8234 | CG2 | ILE | C | 131 | 13.131 | 9.596 | 27.554 | 1.00 | 54.73 | C |
| ATOM | 8235 | CG1 | ILE | C | 131 | 14.903 | 11.239 | 26.826 | 1.00 | 50.69 | C |
| ATOM | 8236 | CD1 | ILE | C | 131 | 15.129 | 10.600 | 25.478 | 1.00 | 49.30 | C |
| ATOM | 8237 | C | ILE | C | 131 | 15.726 | 11.516 | 29.616 | 1.00 | 40.72 | C |
| ATOM | 8238 | O | ILE | C | 131 | 15.980 | 12.711 | 29.470 | 1.00 | 41.44 | C |
| ATOM | 8239 | N | ASN | C | 132 | 16.618 | 10.647 | 30.076 | 1.00 | 62.23 | C |
| ATOM | 8240 | CA | ASN | C | 132 | 17.952 | 11.075 | 30.473 | 1.00 | 59.23 | C |
| ATOM | 8241 | CB | ASN | C | 132 | 18.393 | 10.286 | 31.693 | 1.00 | 56.93 | C |
| ATOM | 8242 | CG | ASN | C | 132 | 18.574 | 8.824 | 31.386 | 1.00 | 55.63 | C |
| ATOM | 8243 | OD1 | ASN | C | 132 | 17.779 | 8.228 | 30.661 | 1.00 | 57.32 | C |
| ATOM | 8244 | ND2 | ASN | C | 132 | 19.620 | 8.227 | 31.939 | 1.00 | 53.46 | C |
| ATOM | 8245 | C | ASN | C | 132 | 19.003 | 10.932 | 29.372 | 1.00 | 56.88 | C |
| ATOM | 8246 | O | ASN | C | 132 | 19.976 | 11.676 | 29.365 | 1.00 | 60.05 | C |
| ATOM | 8247 | N | ARG | C | 133 | 18.816 | 9.985 | 28.451 | 1.00 | 70.42 | C |
| ATOM | 8248 | CA | ARG | C | 133 | 19.775 | 9.768 | 27.357 | 1.00 | 66.01 | C |
| ATOM | 8249 | CB | ARG | C | 133 | 20.372 | 8.360 | 27.447 | 1.00 | 87.13 | C |
| ATOM | 8250 | CG | ARG | C | 133 | 21.136 | 8.088 | 28.727 | 1.00 | 89.71 | C |
| ATOM | 8251 | CD | ARG | C | 133 | 22.265 | 9.083 | 28.913 | 1.00 | 94.95 | C |
| ATOM | 8252 | NE | ARG | C | 133 | 23.223 | 9.050 | 27.810 | 1.00 | 92.81 | C |
| ATOM | 8253 | CZ | ARG | C | 133 | 24.089 | 8.065 | 27.592 | 1.00 | 93.91 | C |
| ATOM | 8254 | NH1 | ARG | C | 133 | 24.920 | 8.128 | 26.559 | 1.00 | 96.59 | C |
| ATOM | 8255 | NH2 | ARG | C | 133 | 24.131 | 7.019 | 28.410 | 1.00 | 93.20 | C |
| ATOM | 8256 | C | ARG | C | 133 | 19.181 | 9.971 | 25.959 | 1.00 | 70.60 | C |
| ATOM | 8257 | O | ARG | C | 133 | 19.049 | 9.024 | 25.187 | 1.00 | 72.96 | C |
| ATOM | 8258 | N | PRO | C | 134 | 18.836 | 11.219 | 25.611 | 1.00 | 58.27 | C |
| ATOM | 8259 | CD | PRO | C | 134 | 19.069 | 12.455 | 26.372 | 1.00 | 56.01 | C |
| ATOM | 8260 | CA | PRO | C | 134 | 18.259 | 11.530 | 24.300 | 1.00 | 61.99 | C |
| ATOM | 8261 | CB | PRO | C | 134 | 18.031 | 13.039 | 24.368 | 1.00 | 58.73 | C |
| ATOM | 8262 | CG | PRO | C | 134 | 19.114 | 13.487 | 25.269 | 1.00 | 60.73 | C |
| ATOM | 8263 | C | PRO | C | 134 | 19.154 | 11.140 | 23.141 | 1.00 | 67.17 | C |
| ATOM | 8264 | O | PRO | C | 134 | 18.723 | 11.146 | 21.993 | 1.00 | 70.21 | C |
| ATOM | 8265 | N | ASP | C | 135 | 20.401 | 10.806 | 23.439 | 1.00 | 71.52 | C |
| ATOM | 8266 | CA | ASP | C | 135 | 21.322 | 10.406 | 22.392 | 1.00 | 67.98 | C |
| ATOM | 8267 | CB | ASP | C | 135 | 22.766 | 10.559 | 22.875 | 1.00 | 91.25 | C |
| ATOM | 8268 | CG | ASP | C | 135 | 22.963 | 10.070 | 24.290 | 1.00 | 90.72 | C |
| ATOM | 8269 | OD1 | ASP | C | 135 | 22.999 | 8.845 | 24.495 | 1.00 | 86.66 | C |
| ATOM | 8270 | OD2 | ASP | C | 135 | 23.074 | 10.912 | 25.205 | 1.00 | 92.30 | C |
| ATOM | 8271 | C | ASP | C | 135 | 21.045 | 8.975 | 21.929 | 1.00 | 66.69 | C |
| ATOM | 8272 | O | ASP | C | 135 | 21.547 | 8.545 | 20.894 | 1.00 | 73.38 | C |
| ATOM | 8273 | N | TYR | C | 136 | 20.229 | 8.245 | 22.684 | 1.00 | 60.90 | C |
| ATOM | 8274 | CA | TYR | C | 136 | 19.883 | 6.869 | 22.333 | 1.00 | 62.11 | C |
| ATOM | 8275 | CB | TYR | C | 136 | 19.997 | 5.965 | 23.555 | 1.00 | 78.49 | C |
| ATOM | 8276 | CG | TYR | C | 136 | 21.402 | 5.610 | 23.967 | 1.00 | 78.10 | C |
| ATOM | 8277 | CD1 | TYR | C | 136 | 21.878 | 5.937 | 25.236 | 1.00 | 77.70 | C |
| ATOM | 8278 | CE1 | TYR | C | 136 | 23.155 | 5.565 | 25.646 | 1.00 | 80.25 | C |
| ATOM | 8279 | CD2 | TYR | C | 136 | 22.239 | 4.902 | 23.112 | 1.00 | 80.27 | C |
| ATOM | 8280 | CE2 | TYR | C | 136 | 23.518 | 4.522 | 23.513 | 1.00 | 79.74 | C |
| ATOM | 8281 | CZ | TYR | C | 136 | 23.967 | 4.858 | 24.782 | 1.00 | 81.04 | C |
| ATOM | 8282 | OH | TYR | C | 136 | 25.222 | 4.480 | 25.192 | 1.00 | 84.88 | C |
| ATOM | 8283 | C | TYR | C | 136 | 18.454 | 6.788 | 21.801 | 1.00 | 64.02 | C |
| ATOM | 8284 | O | TYR | C | 136 | 17.940 | 5.702 | 21.528 | 1.00 | 66.90 | C |
| ATOM | 8285 | N | LEU | C | 137 | 17.815 | 7.941 | 21.654 | 1.00 | 71.75 | C |
| ATOM | 8286 | CA | LEU | C | 137 | 16.442 | 7.997 | 21.184 | 1.00 | 68.46 | C |
| ATOM | 8287 | CB | LEU | C | 137 | 15.933 | 9.430 | 21.250 | 1.00 | 67.39 | C |
| ATOM | 8288 | CG | LEU | C | 137 | 14.730 | 9.696 | 22.144 | 1.00 | 65.12 | C |
| ATOM | 8289 | CD1 | LEU | C | 137 | 14.239 | 11.106 | 21.872 | 1.00 | 67.26 | C |
| ATOM | 8290 | CD2 | LEU | C | 137 | 13.626 | 8.689 | 21.866 | 1.00 | 68.88 | C |
| ATOM | 8291 | C | LEU | C | 137 | 16.246 | 7.494 | 19.767 | 1.00 | 67.83 | C |
| ATOM | 8292 | O | LEU | C | 137 | 16.780 | 8.086 | 18.830 | 1.00 | 66.68 | C |
| ATOM | 8293 | N | LEU | C | 138 | 15.483 | 6.410 | 19.603 | 1.00 | 61.12 | C |
| ATOM | 8294 | CA | LEU | C | 138 | 15.192 | 5.897 | 18.262 | 1.00 | 58.92 | C |
| ATOM | 8295 | CB | LEU | C | 138 | 15.253 | 4.366 | 18.192 | 1.00 | 67.88 | C |
| ATOM | 8296 | CG | LEU | C | 138 | 14.679 | 3.829 | 16.861 | 1.00 | 64.00 | C |
| ATOM | 8297 | CD1 | LEU | C | 138 | 15.275 | 4.597 | 15.688 | 1.00 | 65.57 | C |
| ATOM | 8298 | CD2 | LEU | C | 138 | 14.952 | 2.346 | 16.712 | 1.00 | 66.92 | C |
| ATOM | 8299 | C | LEU | C | 138 | 13.811 | 6.346 | 17.800 | 1.00 | 62.27 | C |
| ATOM | 8300 | O | LEU | C | 138 | 12.799 | 5.740 | 18.145 | 1.00 | 61.42 | C |
| ATOM | 8301 | N | PHE | C | 139 | 13.778 | 7.419 | 17.024 | 1.00 | 64.25 | C |
| ATOM | 8302 | CA | PHE | C | 139 | 12.527 | 7.929 | 16.497 | 1.00 | 65.63 | C |
| ATOM | 8303 | CB | PHE | C | 139 | 12.287 | 9.366 | 16.988 | 1.00 | 92.97 | C |
| ATOM | 8304 | CG | PHE | C | 139 | 13.493 | 10.261 | 16.899 | 1.00 | 92.12 | C |
| ATOM | 8305 | CD1 | PHE | C | 139 | 13.638 | 11.324 | 17.782 | 1.00 | 91.65 | C |
| ATOM | 8306 | CD2 | PHE | C | 139 | 14.470 | 10.062 | 15.930 | 1.00 | 94.73 | C |
| ATOM | 8307 | CE1 | PHE | C | 139 | 14.738 | 12.174 | 17.703 | 1.00 | 96.29 | C |
| ATOM | 8308 | CE2 | PHE | C | 139 | 15.572 | 10.907 | 15.843 | 1.00 | 92.27 | C |
| ATOM | 8309 | CZ | PHE | C | 139 | 15.705 | 11.965 | 16.732 | 1.00 | 96.21 | C |
| ATOM | 8310 | C | PHE | C | 139 | 12.583 | 7.857 | 14.973 | 1.00 | 69.66 | C |
| ATOM | 8311 | O | PHE | C | 139 | 12.102 | 8.748 | 14.269 | 1.00 | 69.76 | C |
| ATOM | 8312 | N | ALA | C | 140 | 13.177 | 6.774 | 14.476 | 1.00 | 68.80 | C |
| ATOM | 8313 | CA | ALA | C | 140 | 13.326 | 6.564 | 13.041 | 1.00 | 70.10 | C |
| ATOM | 8314 | CB | ALA | C | 140 | 14.445 | 5.574 | 12.779 | 1.00 | 73.35 | C |
| ATOM | 8315 | C | ALA | C | 140 | 12.051 | 6.075 | 12.371 | 1.00 | 70.49 | C |
| ATOM | 8316 | O | ALA | C | 140 | 11.670 | 6.584 | 11.314 | 1.00 | 72.38 | C |
| ATOM | 8317 | N | GLU | C | 141 | 11.398 | 5.088 | 12.984 | 1.00 | 86.20 | C |
| ATOM | 8318 | CA | GLU | C | 141 | 10.177 | 4.528 | 12.421 | 1.00 | 89.38 | C |
| ATOM | 8319 | CB | GLU | C | 141 | 9.812 | 3.210 | 13.117 | 1.00 | 200.84 | C |
| ATOM | 8320 | CG | GLU | C | 141 | 8.628 | 2.470 | 12.483 | 1.00 | 200.84 | C |
| ATOM | 8321 | CD | GLU | C | 141 | 7.272 | 2.959 | 12.978 | 1.00 | 200.84 | C |
| ATOM | 8322 | OE1 | GLU | C | 141 | 6.277 | 2.812 | 12.238 | 1.00 | 200.84 | C |
| ATOM | 8323 | OE2 | GLU | C | 141 | 7.194 | 3.476 | 14.113 | 1.00 | 200.84 | C |
| ATOM | 8324 | C | GLU | C | 141 | 8.979 | 5.474 | 12.444 | 1.00 | 91.48 | C |
| ATOM | 8325 | O | GLU | C | 141 | 8.701 | 6.134 | 11.447 | 1.00 | 95.16 | C |
| ATOM | 8326 | N | SER | C | 142 | 8.272 | 5.559 | 13.564 | 1.00 | 130.57 | C |
| ATOM | 8327 | CA | SER | C | 142 | 7.095 | 6.415 | 13.594 | 1.00 | 133.56 | C |
| ATOM | 8328 | CB | SER | C | 142 | 6.133 | 5.968 | 12.491 | 1.00 | 46.70 | C |
| ATOM | 8329 | OG | SER | C | 142 | 5.149 | 6.948 | 12.234 | 1.00 | 47.58 | C |
| ATOM | 8330 | C | SER | C | 142 | 6.360 | 6.421 | 14.934 | 1.00 | 134.88 | C |
| ATOM | 8331 | O | SER | C | 142 | 6.499 | 7.361 | 15.725 | 1.00 | 139.97 | C |
| ATOM | 8332 | N | GLY | C | 143 | 5.558 | 5.387 | 15.181 | 1.00 | 46.99 | C |
| ATOM | 8333 | CA | GLY | C | 143 | 4.835 | 5.338 | 16.440 | 1.00 | 44.46 | C |
| ATOM | 8334 | C | GLY | C | 143 | 5.677 | 4.737 | 17.557 | 1.00 | 42.53 | C |
| ATOM | 8335 | O | GLY | C | 143 | 5.152 | 4.002 | 18.394 | 1.00 | 44.52 | C |
| ATOM | 8336 | N | GLN | C | 144 | 6.973 | 5.056 | 17.573 | 1.00 | 50.15 | C |
| ATOM | 8337 | CA | GLN | C | 144 | 7.904 | 4.525 | 18.565 | 1.00 | 44.68 | C |
| ATOM | 8338 | CB | GLN | C | 144 | 9.265 | 4.254 | 17.921 | 1.00 | 68.35 | C |
| ATOM | 8339 | CG | GLN | C | 144 | 9.195 | 3.502 | 16.611 | 1.00 | 67.63 | C |
| ATOM | 8340 | CD | GLN | C | 144 | 10.566 | 3.131 | 16.072 | 1.00 | 67.60 | C |
| ATOM | 8341 | OE1 | GLN | C | 144 | 11.464 | 3.971 | 15.983 | 1.00 | 73.17 | C |
| ATOM | 8342 | NE2 | GLN | C | 144 | 10.729 | 1.867 | 15.696 | 1.00 | 66.14 | C |
| ATOM | 8343 | C | GLN | C | 144 | 8.108 | 5.442 | 19.760 | 1.00 | 46.48 | C |
| ATOM | 8344 | O | GLN | C | 144 | 8.538 | 4.999 | 20.826 | 1.00 | 48.79 | C |
| ATOM | 8345 | N | VAL | C | 145 | 7.813 | 6.720 | 19.582 | 1.00 | 50.23 | C |
| ATOM | 8346 | CA | VAL | C | 145 | 7.980 | 7.679 | 20.661 | 1.00 | 48.48 | C |
| ATOM | 8347 | CB | VAL | C | 145 | 9.281 | 8.499 | 20.469 | 1.00 | 42.36 | C |
| ATOM | 8348 | CG1 | VAL | C | 145 | 9.468 | 9.482 | 21.614 | 1.00 | 40.52 | C |
| ATOM | 8349 | CG2 | VAL | C | 145 | 10.465 | 7.568 | 20.384 | 1.00 | 39.44 | C |
| ATOM | 8350 | C | VAL | C | 145 | 6.775 | 8.619 | 20.677 | 1.00 | 49.59 | C |
| ATOM | 8351 | O | VAL | C | 145 | 6.551 | 9.380 | 19.736 | 1.00 | 52.27 | C |
| ATOM | 8352 | N | TYR | C | 146 | 5.985 | 8.549 | 21.742 | 1.00 | 41.59 | C |
| ATOM | 8353 | CA | TYR | C | 146 | 4.810 | 9.401 | 21.864 | 1.00 | 39.80 | C |
| ATOM | 8354 | CB | TYR | C | 146 | 3.588 | 8.734 | 21.221 | 1.00 | 46.97 | C |
| ATOM | 8355 | CG | TYR | C | 146 | 3.370 | 7.299 | 21.655 | 1.00 | 47.00 | C |
| ATOM | 8356 | CD1 | TYR | C | 146 | 4.016 | 6.245 | 21.003 | 1.00 | 46.39 | C |
| ATOM | 8357 | CE1 | TYR | C | 146 | 3.848 | 4.931 | 21.426 | 1.00 | 45.18 | C |
| ATOM | 8358 | CD2 | TYR | C | 146 | 2.544 | 6.993 | 22.746 | 1.00 | 42.59 | C |
| ATOM | 8359 | CE2 | TYR | C | 146 | 2.373 | 5.679 | 23.174 | 1.00 | 44.64 | C |
| ATOM | 8360 | CZ | TYR | C | 146 | 3.024 | 4.657 | 22.514 | 1.00 | 44.68 | C |
| ATOM | 8361 | OH | TYR | C | 146 | 2.855 | 3.367 | 22.956 | 1.00 | 45.66 | C |
| ATOM | 8362 | C | TYR | C | 146 | 4.510 | 9.771 | 23.318 | 1.00 | 38.11 | C |
| ATOM | 8363 | O | TYR | C | 146 | 5.047 | 9.192 | 24.267 | 1.00 | 36.28 | C |
| ATOM | 8364 | N | PHE | C | 147 | 3.634 | 10.752 | 23.468 | 1.00 | 33.61 | C |
| ATOM | 8365 | CA | PHE | C | 147 | 3.251 | 11.277 | 24.762 | 1.00 | 33.13 | C |
| ATOM | 8366 | CB | PHE | C | 147 | 4.270 | 12.355 | 25.140 | 1.00 | 31.34 | C |
| ATOM | 8367 | CG | PHE | C | 147 | 3.903 | 13.183 | 26.330 | 1.00 | 30.39 | C |
| ATOM | 8368 | CD1 | PHE | C | 147 | 3.261 | 12.627 | 27.423 | 1.00 | 30.88 | C |
| ATOM | 8369 | CD2 | PHE | C | 147 | 4.292 | 14.528 | 26.391 | 1.00 | 32.72 | C |
| ATOM | 8370 | CE1 | PHE | C | 147 | 3.017 | 13.394 | 28.567 | 1.00 | 31.21 | C |
| ATOM | 8371 | CE2 | PHE | C | 147 | 4.051 | 15.302 | 27.530 | 1.00 | 31.29 | C |
| ATOM | 8372 | CZ | PHE | C | 147 | 3.414 | 14.729 | 28.621 | 1.00 | 32.13 | C |
| ATOM | 8373 | C | PHE | C | 147 | 1.857 | 11.855 | 24.572 | 1.00 | 34.67 | C |
| ATOM | 8374 | O | PHE | C | 147 | 1.614 | 12.614 | 23.643 | 1.00 | 32.15 | C |
| ATOM | 8375 | N | GLY | C | 148 | 0.924 | 11.467 | 25.423 | 1.00 | 33.63 | C |
| ATOM | 8376 | CA | GLY | C | 148 | −0.420 | 11.990 | 25.284 | 1.00 | 38.11 | C |
| ATOM | 8377 | C | GLY | C | 148 | −1.238 | 11.976 | 26.564 | 1.00 | 40.33 | C |
| ATOM | 8378 | O | GLY | C | 148 | −0.846 | 11.398 | 27.576 | 1.00 | 42.94 | C |
| ATOM | 8379 | N | ILE | C | 149 | −2.384 | 12.643 | 26.506 | 1.00 | 35.68 | C |
| ATOM | 8380 | CA | ILE | C | 149 | −3.305 | 12.705 | 27.620 | 1.00 | 34.09 | C |
| ATOM | 8381 | CB | ILE | C | 149 | −2.983 | 13.869 | 28.571 | 1.00 | 43.66 | C |
| ATOM | 8382 | CG2 | ILE | C | 149 | −1.511 | 13.839 | 28.952 | 1.00 | 44.21 | C |
| ATOM | 8383 | CG1 | ILE | C | 149 | −3.305 | 15.202 | 27.903 | 1.00 | 39.70 | C |
| ATOM | 8384 | CD1 | ILE | C | 149 | −3.165 | 16.363 | 28.849 | 1.00 | 42.95 | C |
| ATOM | 8385 | C | ILE | C | 149 | −4.720 | 12.880 | 27.084 | 1.00 | 36.13 | C |
| ATOM | 8386 | O | ILE | C | 149 | −4.939 | 13.483 | 26.036 | 1.00 | 41.30 | C |
| ATOM | 8387 | N | ILE | C | 150 | −5.678 | 12.338 | 27.812 | 1.00 | 48.87 | C |
| ATOM | 8388 | CA | ILE | C | 150 | −7.074 | 12.427 | 27.433 | 1.00 | 49.38 | C |
| ATOM | 8389 | CB | ILE | C | 150 | −7.538 | 11.104 | 26.752 | 1.00 | 37.00 | C |
| ATOM | 8390 | CG2 | ILE | C | 150 | −7.234 | 9.915 | 27.644 | 1.00 | 40.30 | C |
| ATOM | 8391 | CG1 | ILE | C | 150 | −9.032 | 11.155 | 26.442 | 1.00 | 34.99 | C |
| ATOM | 8392 | CD1 | ILE | C | 150 | −9.395 | 12.096 | 25.336 | 1.00 | 33.00 | C |
| ATOM | 8393 | C | ILE | C | 150 | −7.859 | 12.670 | 28.723 | 1.00 | 50.72 | C |
| ATOM | 8394 | O | ILE | C | 150 | −7.601 | 12.035 | 29.746 | 1.00 | 49.83 | C |
| ATOM | 8395 | N | ALA | C | 151 | −8.794 | 13.609 | 28.687 | 1.00 | 46.42 | C |
| ATOM | 8396 | CA | ALA | C | 151 | −9.598 | 13.900 | 29.861 | 1.00 | 45.16 | C |
| ATOM | 8397 | CB | ALA | C | 151 | −10.171 | 15.289 | 29.753 | 1.00 | 23.47 | C |
| ATOM | 8398 | C | ALA | C | 151 | −10.724 | 12.864 | 30.007 | 1.00 | 46.12 | C |
| ATOM | 8399 | O | ALA | C | 151 | −11.526 | 12.656 | 29.097 | 1.00 | 48.50 | C |
| ATOM | 8400 | N | LEU | C | 152 | −10.769 | 12.205 | 31.157 | 1.00 | 73.18 | C |
| ATOM | 8401 | CA | LEU | C | 152 | −11.786 | 11.201 | 31.402 | 1.00 | 77.16 | C |
| ATOM | 8402 | CB | LEU | C | 152 | −11.150 | 9.965 | 32.021 | 1.00 | 54.01 | C |
| ATOM | 8403 | CG | LEU | C | 152 | −10.100 | 9.296 | 31.146 | 1.00 | 55.26 | C |
| ATOM | 8404 | CD1 | LEU | C | 152 | −9.494 | 8.118 | 31.885 | 1.00 | 52.95 | C |
| ATOM | 8405 | CD2 | LEU | C | 152 | −10.736 | 8.840 | 29.856 | 1.00 | 54.46 | C |
| ATOM | 8406 | C | LEU | C | 152 | −12.870 | 11.739 | 32.319 | 1.00 | 80.01 | C |
| ATOM | 8407 | O | LEU | C | 152 | −13.998 | 11.963 | 31.886 | 1.00 | 85.87 | C |
| ATOM | 8408 | N | ILE | C | 153 | 8.364 | 23.567 | 5.314 | 1.00 | 47.98 | C |
| ATOM | 8409 | CA | ILE | C | 153 | 8.537 | 22.788 | 4.095 | 1.00 | 47.98 | C |
| ATOM | 8410 | CB | ILE | C | 153 | 9.039 | 21.361 | 4.396 | 1.00 | 52.12 | C |
| ATOM | 8411 | CG2 | ILE | C | 153 | 10.480 | 21.411 | 4.853 | 1.00 | 48.90 | C |
| ATOM | 8412 | CG1 | ILE | C | 153 | 8.159 | 20.711 | 5.462 | 1.00 | 48.90 | C |
| ATOM | 8413 | CD1 | ILE | C | 153 | 8.588 | 19.309 | 5.810 | 1.00 | 48.90 | C |
| ATOM | 8414 | C | ILE | C | 153 | 7.244 | 22.701 | 3.307 | 1.00 | 47.98 | C |
| ATOM | 8415 | O | ILE | C | 153 | 6.152 | 22.907 | 3.842 | 1.00 | 47.98 | C |
| ATOM | 8416 | N | THR | C | 154 | 7.382 | 22.369 | 2.030 | 1.00 | 57.40 | C |
| ATOM | 8417 | CA | THR | C | 154 | 6.248 | 22.290 | 1.127 | 1.00 | 57.40 | C |
| ATOM | 8418 | CB | THR | C | 154 | 6.100 | 23.630 | 0.398 | 1.00 | 50.74 | C |
| ATOM | 8419 | OG1 | THR | C | 154 | 5.024 | 24.360 | 0.991 | 1.00 | 61.02 | C |
| ATOM | 8420 | CG2 | THR | C | 154 | 5.865 | 23.433 | −1.111 | 1.00 | 61.02 | C |
| ATOM | 8421 | C | THR | C | 154 | 6.331 | 21.176 | 0.088 | 1.00 | 57.40 | C |
| ATOM | 8422 | O | THR | C | 154 | 7.410 | 20.853 | −0.407 | 1.00 | 57.40 | C |
| ATOM | 8423 | N | LEU | C | 155 | 5.180 | 20.586 | −0.227 | 1.00 | 63.14 | C |
| ATOM | 8424 | CA | LEU | C | 155 | 5.105 | 19.549 | −1.250 | 1.00 | 63.14 | C |
| ATOM | 8425 | CB | LEU | C | 155 | 4.898 | 18.161 | −0.642 | 1.00 | 61.37 | C |
| ATOM | 8426 | CG | LEU | C | 155 | 4.910 | 17.027 | −1.681 | 1.00 | 57.95 | C |
| ATOM | 8427 | CD1 | LEU | C | 155 | 6.190 | 17.077 | −2.503 | 1.00 | 57.95 | C |
| ATOM | 8428 | CD2 | LEU | C | 155 | 4.799 | 15.691 | −0.990 | 1.00 | 57.95 | C |
| ATOM | 8429 | C | LEU | C | 155 | 3.920 | 19.918 | −2.134 | 1.00 | 63.14 | C |
| ATOM | 8430 | O | LEU | C | 155 | 2.782 | 19.964 | −1.662 | 1.00 | 63.14 | C |
| ATOM | 8431 | N | LYS | C | 156 | 4.190 | 20.204 | −3.409 | 1.00 | 66.44 | C |
| ATOM | 8432 | CA | LYS | C | 156 | 3.136 | 20.593 | −4.337 | 1.00 | 66.44 | C |
| ATOM | 8433 | CB | LYS | C | 156 | 3.287 | 22.068 | −4.710 | 1.00 | 70.82 | C |
| ATOM | 8434 | CG | LYS | C | 156 | 2.143 | 22.586 | −5.565 | 1.00 | 73.01 | C |
| ATOM | 8435 | CD | LYS | C | 156 | 2.282 | 24.067 | −5.933 | 1.00 | 73.01 | C |
| ATOM | 8436 | CE | LYS | C | 156 | 3.430 | 24.318 | −6.910 | 1.00 | 73.01 | C |
| ATOM | 8437 | NZ | LYS | C | 156 | 3.472 | 25.732 | −7.383 | 1.00 | 73.01 | C |
| ATOM | 8438 | C | LYS | C | 156 | 3.090 | 19.761 | −5.612 | 1.00 | 66.44 | C |
| ATOM | 8439 | O | LYS | C | 156 | 4.121 | 19.535 | −6.249 | 1.00 | 66.44 | C |
| ATOM | 8440 | N | TYR | C | 157 | 1.885 | 19.307 | −5.968 | 1.00 | 53.42 | C |
| ATOM | 8441 | CA | TYR | C | 157 | 1.642 | 18.526 | −7.181 | 1.00 | 53.42 | C |
| ATOM | 8442 | CB | TYR | C | 157 | 0.922 | 17.219 | −6.848 | 1.00 | 66.52 | C |
| ATOM | 8443 | CG | TYR | C | 157 | 1.801 | 16.149 | −6.234 | 1.00 | 48.86 | C |
| ATOM | 8444 | CD1 | TYR | C | 157 | 3.123 | 16.418 | −5.882 | 1.00 | 48.86 | C |
| ATOM | 8445 | CE1 | TYR | C | 157 | 3.930 | 15.435 | −5.308 | 1.00 | 48.86 | C |
| ATOM | 8446 | CD2 | TYR | C | 157 | 1.305 | 14.866 | −5.999 | 1.00 | 48.86 | C |
| ATOM | 8447 | CE2 | TYR | C | 157 | 2.105 | 13.874 | −5.426 | 1.00 | 48.86 | C |
| ATOM | 8448 | CZ | TYR | C | 157 | 3.416 | 14.164 | −5.082 | 1.00 | 48.86 | C |
| ATOM | 8449 | OH | TYR | C | 157 | 4.219 | 13.202 | −4.509 | 1.00 | 48.86 | C |
| ATOM | 8450 | C | TYR | C | 157 | 0.765 | 19.377 | −8.105 | 1.00 | 53.42 | C |
| ATOM | 8451 | O | TYR | C | 157 | −0.287 | 19.883 | −7.682 | 1.00 | 53.42 | C |
| ATOM | 8452 | N | ASN | C | 158 | 1.205 | 19.537 | −9.356 | 1.00 | 65.75 | C |
| ATOM | 8453 | CA | ASN | C | 158 | 0.485 | 20.347 | −10.345 | 1.00 | 65.75 | C |
| ATOM | 8454 | CB | ASN | C | 158 | 1.294 | 21.627 | −10.648 | 1.00 | 57.21 | C |
| ATOM | 8455 | CG | ASN | C | 158 | 0.586 | 22.581 | −11.611 | 1.00 | 67.91 | C |
| ATOM | 8456 | OD1 | ASN | C | 158 | −0.495 | 22.279 | −12.123 | 1.00 | 67.91 | C |
| ATOM | 8457 | ND2 | ASN | C | 158 | 1.215 | 23.737 | −11.852 | 1.00 | 67.91 | C |
| ATOM | 8458 | C | ASN | C | 158 | 0.254 | 19.558 | −11.635 | 1.00 | 65.75 | C |
| ATOM | 8459 | O | ASN | C | 158 | 1.203 | 19.198 | −12.332 | 1.00 | 65.75 | C |
| ATOM | 8460 | N | TYR | C | 159 | −1.009 | 19.271 | −11.932 | 1.00 | 53.68 | C |
| ATOM | 8461 | CA | TYR | C | 159 | −1.358 | 18.558 | −13.152 | 1.00 | 53.68 | C |
| ATOM | 8462 | CB | TYR | C | 159 | −2.337 | 17.412 | −12.894 | 1.00 | 64.95 | C |
| ATOM | 8463 | CG | TYR | C | 159 | −1.844 | 16.285 | −12.024 | 1.00 | 68.51 | C |
| ATOM | 8464 | CD1 | TYR | C | 159 | −1.901 | 16.378 | −10.634 | 1.00 | 68.51 | C |
| ATOM | 8465 | CE1 | TYR | C | 159 | −1.528 | 15.311 | −9.826 | 1.00 | 68.51 | C |
| ATOM | 8466 | CD2 | TYR | C | 159 | −1.392 | 15.094 | −12.589 | 1.00 | 68.51 | C |
| ATOM | 8467 | CE2 | TYR | C | 159 | −1.015 | 14.019 | −11.790 | 1.00 | 68.51 | C |
| ATOM | 8468 | CZ | TYR | C | 159 | −1.088 | 14.133 | −10.405 | 1.00 | 68.51 | C |
| ATOM | 8469 | OH | TYR | C | 159 | −0.732 | 13.070 | −9.603 | 1.00 | 68.51 | C |
| ATOM | 8470 | C | TYR | C | 159 | −2.057 | 19.576 | −14.041 | 1.00 | 53.68 | C |
| ATOM | 8471 | O | TYR | C | 159 | −2.995 | 20.263 | −13.597 | 1.00 | 53.68 | C |
| ATOM | 8472 | N | THR | C | 160 | −1.618 | 19.677 | −15.293 | 1.00 | 80.04 | C |
| ATOM | 8473 | CA | THR | C | 160 | −2.227 | 20.638 | −16.204 | 1.00 | 80.04 | C |
| ATOM | 8474 | CB | THR | C | 160 | −1.422 | 21.948 | −16.219 | 1.00 | 95.91 | C |
| ATOM | 8475 | OG1 | THR | C | 160 | −1.256 | 22.425 | −14.877 | 1.00 | 83.25 | C |
| ATOM | 8476 | CG2 | THR | C | 160 | −2.149 | 23.001 | −17.036 | 1.00 | 83.25 | C |
| ATOM | 8477 | C | THR | C | 160 | −2.392 | 20.178 | −17.649 | 1.00 | 80.04 | C |
| ATOM | 8478 | O | THR | C | 160 | −1.431 | 19.727 | −18.278 | 1.00 | 80.04 | C |
| ATOM | 8479 | N | VAL | C | 161 | −3.619 | 20.275 | −18.162 | 1.00 | 77.22 | C |
| ATOM | 8480 | CA | VAL | C | 161 | −3.882 | 19.934 | −19.558 | 1.00 | 77.22 | C |
| ATOM | 8481 | CB | VAL | C | 161 | −5.011 | 18.884 | −19.744 | 1.00 | 101.00 | C |
| ATOM | 8482 | CG1 | VAL | C | 161 | −4.752 | 17.689 | −18.863 | 1.00 | 85.51 | C |
| ATOM | 8483 | CG2 | VAL | C | 161 | −6.362 | 19.493 | −19.457 | 1.00 | 85.51 | C |
| ATOM | 8484 | C | VAL | C | 161 | −4.294 | 21.241 | −20.223 | 1.00 | 77.22 | C |
| ATOM | 8485 | O | VAL | C | 161 | −5.084 | 22.015 | −19.662 | 1.00 | 77.22 | C |
| ATOM | 8486 | N | THR | C | 162 | −3.744 | 21.493 | −21.407 | 1.00 | 124.39 | C |
| ATOM | 8487 | CA | THR | C | 162 | −4.023 | 22.726 | −22.136 | 1.00 | 124.39 | C |
| ATOM | 8488 | CB | THR | C | 162 | −2.756 | 23.588 | −22.246 | 1.00 | 109.03 | C |
| ATOM | 8489 | OG1 | THR | C | 162 | −1.960 | 23.408 | −21.070 | 1.00 | 80.36 | C |
| ATOM | 8490 | CG2 | THR | C | 162 | −3.118 | 25.059 | −22.382 | 1.00 | 80.36 | C |
| ATOM | 8491 | C | THR | C | 162 | −4.515 | 22.445 | −23.546 | 1.00 | 124.39 | C |
| ATOM | 8492 | O | THR | C | 162 | −4.138 | 21.443 | −24.154 | 1.00 | 124.39 | C |
| ATOM | 8493 | N | LEU | C | 163 | −5.354 | 23.332 | −24.069 | 1.00 | 109.84 | C |
| ATOM | 8494 | CA | LEU | C | 163 | −5.859 | 23.160 | −25.418 | 1.00 | 109.84 | C |
| ATOM | 8495 | CB | LEU | C | 163 | −7.138 | 23.967 | −25.630 | 1.00 | 131.26 | C |
| ATOM | 8496 | CG | LEU | C | 163 | −8.321 | 23.124 | −26.117 | 1.00 | 79.35 | C |
| ATOM | 8497 | CD1 | LEU | C | 163 | −9.476 | 24.029 | −26.496 | 1.00 | 79.35 | C |
| ATOM | 8498 | CD2 | LEU | C | 163 | −7.898 | 22.269 | −27.311 | 1.00 | 79.35 | C |
| ATOM | 8499 | C | LEU | C | 163 | −4.800 | 23.615 | −26.408 | 1.00 | 109.84 | C |
| ATOM | 8500 | O | LEU | C | 163 | −4.946 | 23.423 | −27.614 | 1.00 | 109.84 | C |
| ATOM | 8501 | N | LYS | C | 164 | −3.726 | 24.208 | −25.892 | 1.00 | 200.27 | C |
| ATOM | 8502 | CA | LYS | C | 164 | −2.644 | 24.697 | −26.742 | 1.00 | 200.27 | C |
| ATOM | 8503 | CB | LYS | C | 164 | −1.817 | 23.529 | −27.284 | 1.00 | 112.13 | C |
| ATOM | 8504 | CG | LYS | C | 164 | −0.488 | 23.330 | −26.576 | 1.00 | 111.88 | C |
| ATOM | 8505 | CD | LYS | C | 164 | 0.408 | 24.546 | −26.754 | 1.00 | 111.88 | C |
| ATOM | 8506 | CE | LYS | C | 164 | 1.757 | 24.362 | −26.074 | 1.00 | 111.88 | C |
| ATOM | 8507 | NZ | LYS | C | 164 | 2.678 | 25.504 | −26.357 | 1.00 | 111.88 | C |
| ATOM | 8508 | C | LYS | C | 164 | −3.254 | 25.469 | −27.898 | 1.00 | 200.27 | C |
| ATOM | 8509 | O | LYS | C | 164 | −4.403 | 25.904 | −27.812 | 1.00 | 200.27 | C |
| ATOM | 8510 | N | ASP | C | 165 | −2.492 | 25.650 | −28.972 | 1.00 | 197.57 | C |
| ATOM | 8511 | CA | ASP | C | 165 | −3.013 | 26.356 | −30.135 | 1.00 | 197.57 | C |
| ATOM | 8512 | CB | ASP | C | 165 | −1.922 | 26.521 | −31.202 | 1.00 | 129.33 | C |
| ATOM | 8513 | CG | ASP | C | 165 | −1.188 | 25.223 | −31.504 | 1.00 | 129.33 | C |
| ATOM | 8514 | OD1 | ASP | C | 165 | −1.823 | 24.274 | −32.016 | 1.00 | 129.33 | C |
| ATOM | 8515 | OD2 | ASP | C | 165 | 0.030 | 25.152 | −31.229 | 1.00 | 129.33 | C |
| ATOM | 8516 | C | ASP | C | 165 | −4.180 | 25.533 | −30.670 | 1.00 | 197.57 | C |
| ATOM | 8517 | O | ASP | C | 165 | −5.034 | 25.088 | −29.902 | 1.00 | 197.57 | C |
| ATOM | 8518 | N | ASP | C | 166 | −4.230 | 25.314 | −31.975 | 1.00 | 86.28 | C |
| ATOM | 8519 | CA | ASP | C | 166 | −5.325 | 24.530 | −32.513 | 1.00 | 86.28 | C |
| ATOM | 8520 | CB | ASP | C | 166 | −5.755 | 25.096 | −33.859 | 1.00 | 200.84 | C |
| ATOM | 8521 | CG | ASP | C | 166 | −6.345 | 26.483 | −33.730 | 1.00 | 200.84 | C |
| ATOM | 8522 | OD1 | ASP | C | 166 | −5.627 | 27.392 | −33.262 | 1.00 | 200.84 | C |
| ATOM | 8523 | OD2 | ASP | C | 166 | −7.527 | 26.663 | −34.085 | 1.00 | 200.84 | C |
| ATOM | 8524 | C | ASP | C | 166 | −5.004 | 23.046 | −32.632 | 1.00 | 86.28 | C |
| ATOM | 8525 | O | ASP | C | 166 | −4.056 | 22.651 | −33.319 | 1.00 | 86.28 | C |
| ATOM | 8526 | N | GLY | C | 167 | −5.788 | 22.229 | −31.928 | 1.00 | 96.04 | C |
| ATOM | 8527 | CA | GLY | C | 167 | −5.590 | 20.797 | −31.996 | 1.00 | 96.04 | C |
| ATOM | 8528 | C | GLY | C | 167 | −5.730 | 19.956 | −30.745 | 1.00 | 96.04 | C |
| ATOM | 8529 | O | GLY | C | 167 | −6.779 | 19.899 | −30.098 | 1.00 | 96.04 | C |
| ATOM | 8530 | N | LEU | C | 168 | −4.630 | 19.287 | −30.431 | 1.00 | 134.70 | C |
| ATOM | 8531 | CA | LEU | C | 168 | −4.533 | 18.379 | −29.308 | 1.00 | 134.70 | C |
| ATOM | 8532 | CB | LEU | C | 168 | −3.269 | 17.538 | −29.482 | 1.00 | 107.96 | C |
| ATOM | 8533 | CG | LEU | C | 168 | −2.880 | 17.331 | −30.950 | 1.00 | 107.96 | C |
| ATOM | 8534 | CD1 | LEU | C | 168 | −1.534 | 16.636 | −31.039 | 1.00 | 107.96 | C |
| ATOM | 8535 | CD2 | LEU | C | 168 | −3.958 | 16.525 | −31.658 | 1.00 | 107.96 | C |
| ATOM | 8536 | C | LEU | C | 168 | −4.512 | 19.070 | −27.951 | 1.00 | 134.70 | C |
| ATOM | 8537 | O | LEU | C | 168 | −4.453 | 20.296 | −27.852 | 1.00 | 134.70 | C |
| ATOM | 8538 | N | TYR | C | 169 | −4.570 | 18.256 | −26.903 | 1.00 | 93.30 | C |
| ATOM | 8539 | CA | TYR | C | 169 | −4.534 | 18.746 | −25.540 | 1.00 | 93.30 | C |
| ATOM | 8540 | CB | TYR | C | 169 | −5.663 | 18.131 | −24.716 | 1.00 | 70.75 | C |
| ATOM | 8541 | CG | TYR | C | 169 | −7.044 | 18.601 | −25.083 | 1.00 | 70.75 | C |
| ATOM | 8542 | CD1 | TYR | C | 169 | −7.687 | 18.122 | −26.215 | 1.00 | 70.75 | C |
| ATOM | 8543 | CE1 | TYR | C | 169 | −8.968 | 18.553 | −26.547 | 1.00 | 70.75 | C |
| ATOM | 8544 | CD2 | TYR | C | 169 | −7.712 | 19.526 | −24.286 | 1.00 | 70.75 | C |
| ATOM | 8545 | CE2 | TYR | C | 169 | −8.989 | 19.966 | −24.603 | 1.00 | 70.75 | C |
| ATOM | 8546 | CZ | TYR | C | 169 | −9.613 | 19.477 | −25.733 | 1.00 | 70.75 | C |
| ATOM | 8547 | OH | TYR | C | 169 | −10.878 | 19.920 | −26.045 | 1.00 | 70.75 | C |
| ATOM | 8548 | C | TYR | C | 169 | −3.206 | 18.318 | −24.945 | 1.00 | 93.30 | C |
| ATOM | 8549 | O | TYR | C | 169 | −2.897 | 17.126 | −24.908 | 1.00 | 93.30 | C |
| ATOM | 8550 | N | ASP | C | 170 | −2.411 | 19.281 | −24.494 | 1.00 | 76.82 | C |
| ATOM | 8551 | CA | ASP | C | 170 | −1.132 | 18.943 | −23.881 | 1.00 | 76.82 | C |
| ATOM | 8552 | CB | ASP | C | 170 | −0.111 | 20.064 | −24.066 | 1.00 | 107.20 | C |
| ATOM | 8553 | CG | ASP | C | 170 | 0.734 | 19.876 | −25.305 | 1.00 | 107.20 | C |
| ATOM | 8554 | OD1 | ASP | C | 170 | 1.098 | 18.716 | −25.598 | 1.00 | 107.20 | C |
| ATOM | 8555 | OD2 | ASP | C | 170 | 1.047 | 20.881 | −25.975 | 1.00 | 107.20 | C |
| ATOM | 8556 | C | ASP | C | 170 | −1.316 | 18.674 | −22.399 | 1.00 | 76.82 | C |
| ATOM | 8557 | O | ASP | C | 170 | −2.261 | 19.164 | −21.780 | 1.00 | 76.82 | C |
| ATOM | 8558 | N | GLY | C | 171 | −0.412 | 17.883 | −21.838 | 1.00 | 65.00 | C |
| ATOM | 8559 | CA | GLY | C | 171 | −0.500 | 17.571 | −20.432 | 1.00 | 65.00 | C |
| ATOM | 8560 | C | GLY | C | 171 | 0.862 | 17.578 | −19.774 | 1.00 | 65.00 | C |
| ATOM | 8561 | O | GLY | C | 171 | 1.837 | 17.047 | −20.318 | 1.00 | 65.00 | C |
| ATOM | 8562 | N | VAL | C | 172 | 0.938 | 18.208 | −18.607 | 1.00 | 73.07 | C |
| ATOM | 8563 | CA | VAL | C | 172 | 2.178 | 18.246 | −17.844 | 1.00 | 73.07 | C |
| ATOM | 8564 | CB | VAL | C | 172 | 2.927 | 19.586 | −17.946 | 1.00 | 71.73 | C |
| ATOM | 8565 | CG1 | VAL | C | 172 | 3.813 | 19.581 | −19.151 | 1.00 | 63.74 | C |
| ATOM | 8566 | CG2 | VAL | C | 172 | 1.937 | 20.745 | −17.979 | 1.00 | 63.74 | C |
| ATOM | 8567 | C | VAL | C | 172 | 1.883 | 18.041 | −16.383 | 1.00 | 73.07 | C |
| ATOM | 8568 | O | VAL | C | 172 | 0.773 | 18.286 | −15.909 | 1.00 | 73.07 | C |
| ATOM | 8569 | N | PHE | C | 173 | 2.905 | 17.593 | −15.674 | 1.00 | 68.65 | C |
| ATOM | 8570 | CA | PHE | C | 173 | 2.806 | 17.373 | −14.259 | 1.00 | 68.65 | C |
| ATOM | 8571 | CB | PHE | C | 173 | 2.595 | 15.902 | −13.956 | 1.00 | 79.77 | C |
| ATOM | 8572 | CG | PHE | C | 173 | 2.797 | 15.577 | −12.517 | 1.00 | 61.59 | C |
| ATOM | 8573 | CD1 | PHE | C | 173 | 1.827 | 15.916 | −11.581 | 1.00 | 61.59 | C |
| ATOM | 8574 | CD2 | PHE | C | 173 | 3.996 | 15.016 | −12.082 | 1.00 | 61.59 | C |
| ATOM | 8575 | CE1 | PHE | C | 173 | 2.047 | 15.706 | −10.238 | 1.00 | 61.59 | C |
| ATOM | 8576 | CE2 | PHE | C | 173 | 4.229 | 14.801 | −10.740 | 1.00 | 61.59 | C |
| ATOM | 8577 | CZ | PHE | C | 173 | 3.253 | 15.147 | −9.812 | 1.00 | 61.59 | C |
| ATOM | 8578 | C | PHE | C | 173 | 4.107 | 17.814 | −13.609 | 1.00 | 68.65 | C |
| ATOM | 8579 | O | PHE | C | 173 | 5.190 | 17.509 | −14.110 | 1.00 | 68.65 | C |
| ATOM | 8580 | N | TYR | C | 174 | 4.004 | 18.530 | −12.496 | 1.00 | 64.96 | C |
| ATOM | 8581 | CA | TYR | C | 174 | 5.191 | 18.967 | −11.778 | 1.00 | 64.96 | C |
| ATOM | 8582 | CB | TYR | C | 174 | 5.368 | 20.483 | −11.864 | 1.00 | 98.43 | C |
| ATOM | 8583 | CG | TYR | C | 174 | 5.673 | 20.999 | −13.245 | 1.00 | 84.47 | C |
| ATOM | 8584 | CD1 | TYR | C | 174 | 4.696 | 21.658 | −13.993 | 1.00 | 84.47 | C |
| ATOM | 8585 | CE1 | TYR | C | 174 | 4.974 | 22.156 | −15.267 | 1.00 | 84.47 | C |
| ATOM | 8586 | CD2 | TYR | C | 174 | 6.940 | 20.844 | −13.805 | 1.00 | 84.47 | C |
| ATOM | 8587 | CE2 | TYR | C | 174 | 7.228 | 21.336 | −15.081 | 1.00 | 84.47 | C |
| ATOM | 8588 | CZ | TYR | C | 174 | 6.241 | 21.992 | −15.805 | 1.00 | 84.47 | C |
| ATOM | 8589 | OH | TYR | C | 174 | 6.520 | 22.493 | −17.059 | 1.00 | 84.47 | C |
| ATOM | 8590 | C | TYR | C | 174 | 5.099 | 18.576 | −10.313 | 1.00 | 64.96 | C |
| ATOM | 8591 | O | TYR | C | 174 | 4.046 | 18.717 | −9.689 | 1.00 | 64.96 | C |
| ATOM | 8592 | N | ASP | C | 175 | 6.213 | 18.082 | −9.783 | 1.00 | 62.60 | C |
| ATOM | 8593 | CA | ASP | C | 175 | 6.326 | 17.684 | −8.390 | 1.00 | 62.60 | C |
| ATOM | 8594 | CB | ASP | C | 175 | 6.826 | 16.239 | −8.290 | 1.00 | 110.92 | C |
| ATOM | 8595 | CG | ASP | C | 175 | 6.850 | 15.722 | −6.861 | 1.00 | 99.13 | C |
| ATOM | 8596 | OD1 | ASP | C | 175 | 7.082 | 16.523 | −5.936 | 1.00 | 99.13 | C |
| ATOM | 8597 | OD2 | ASP | C | 175 | 6.651 | 14.509 | −6.659 | 1.00 | 99.13 | C |
| ATOM | 8598 | C | ASP | C | 175 | 7.368 | 18.630 | −7.794 | 1.00 | 62.60 | C |
| ATOM | 8599 | O | ASP | C | 175 | 8.548 | 18.569 | −8.162 | 1.00 | 62.60 | C |
| ATOM | 8600 | N | HIS | C | 176 | 6.938 | 19.504 | −6.886 | 1.00 | 60.45 | C |
| ATOM | 8601 | CA | HIS | C | 176 | 7.853 | 20.452 | −6.253 | 1.00 | 60.45 | C |
| ATOM | 8602 | CB | HIS | C | 176 | 7.406 | 21.881 | −6.539 | 1.00 | 101.28 | C |
| ATOM | 8603 | CG | HIS | C | 176 | 7.693 | 22.343 | −7.930 | 1.00 | 70.41 | C |
| ATOM | 8604 | CD2 | HIS | C | 176 | 6.865 | 22.599 | −8.969 | 1.00 | 70.41 | C |
| ATOM | 8605 | ND1 | HIS | C | 176 | 8.969 | 22.627 | −8.371 | 1.00 | 70.41 | C |
| ATOM | 8606 | CE1 | HIS | C | 176 | 8.913 | 23.043 | −9.622 | 1.00 | 70.41 | C |
| ATOM | 8607 | NE2 | HIS | C | 176 | 7.648 | 23.035 | −10.009 | 1.00 | 70.41 | C |
| ATOM | 8608 | C | HIS | C | 176 | 7.964 | 20.292 | −4.743 | 1.00 | 60.45 | C |
| ATOM | 8609 | O | HIS | C | 176 | 6.955 | 20.285 | −4.039 | 1.00 | 60.45 | C |
| ATOM | 8610 | N | TYR | C | 177 | 9.190 | 20.149 | −4.253 | 1.00 | 65.78 | C |
| ATOM | 8611 | CA | TYR | C | 177 | 9.437 | 20.065 | −2.818 | 1.00 | 65.78 | C |
| ATOM | 8612 | CB | TYR | C | 177 | 10.463 | 18.990 | −2.484 | 1.00 | 65.17 | C |
| ATOM | 8613 | CG | TYR | C | 177 | 10.787 | 18.977 | −1.020 | 1.00 | 58.18 | C |
| ATOM | 8614 | CD1 | TYR | C | 177 | 9.826 | 18.601 | −0.081 | 1.00 | 58.18 | C |
| ATOM | 8615 | CE1 | TYR | C | 177 | 10.097 | 18.645 | 1.286 | 1.00 | 58.18 | C |
| ATOM | 8616 | CD2 | TYR | C | 177 | 12.031 | 19.392 | −0.561 | 1.00 | 58.18 | C |
| ATOM | 8617 | CE2 | TYR | C | 177 | 12.314 | 19.440 | 0.800 | 1.00 | 58.18 | C |
| ATOM | 8618 | CZ | TYR | C | 177 | 11.341 | 19.066 | 1.718 | 1.00 | 58.18 | C |
| ATOM | 8619 | OH | TYR | C | 177 | 11.608 | 19.132 | 3.068 | 1.00 | 58.18 | C |
| ATOM | 8620 | C | TYR | C | 177 | 10.033 | 21.434 | −2.533 | 1.00 | 65.78 | C |
| ATOM | 8621 | O | TYR | C | 177 | 11.127 | 21.727 | −2.999 | 1.00 | 65.78 | C |
| ATOM | 8622 | N | ASN | C | 178 | 9.337 | 22.266 | −1.763 | 1.00 | 67.47 | C |
| ATOM | 8623 | CA | ASN | C | 178 | 9.810 | 23.627 | −1.521 | 1.00 | 67.47 | C |
| ATOM | 8624 | CB | ASN | C | 178 | 11.210 | 23.647 | −0.911 | 1.00 | 70.72 | C |
| ATOM | 8625 | CG | ASN | C | 178 | 11.210 | 23.282 | 0.540 | 1.00 | 63.19 | C |
| ATOM | 8626 | OD1 | ASN | C | 178 | 10.155 | 23.212 | 1.170 | 1.00 | 63.19 | C |
| ATOM | 8627 | ND2 | ASN | C | 178 | 12.400 | 23.057 | 1.097 | 1.00 | 63.19 | C |
| ATOM | 8628 | C | ASN | C | 178 | 9.853 | 24.205 | −2.929 | 1.00 | 67.47 | C |
| ATOM | 8629 | O | ASN | C | 178 | 8.914 | 24.012 | −3.702 | 1.00 | 67.47 | C |
| ATOM | 8630 | N | ASP | C | 179 | 10.934 | 24.892 | −3.278 | 1.00 | 73.98 | C |
| ATOM | 8631 | CA | ASP | C | 179 | 11.044 | 25.449 | −4.617 | 1.00 | 73.98 | C |
| ATOM | 8632 | CB | ASP | C | 179 | 11.639 | 26.868 | −4.570 | 1.00 | 65.69 | C |
| ATOM | 8633 | CG | ASP | C | 179 | 12.991 | 26.932 | −3.862 | 1.00 | 69.64 | C |
| ATOM | 8634 | OD1 | ASP | C | 179 | 13.504 | 25.871 | −3.417 | 1.00 | 69.64 | C |
| ATOM | 8635 | OD2 | ASP | C | 179 | 13.536 | 28.060 | −3.758 | 1.00 | 69.64 | C |
| ATOM | 8636 | C | ASP | C | 179 | 11.874 | 24.546 | −5.528 | 1.00 | 73.98 | C |
| ATOM | 8637 | O | ASP | C | 179 | 12.258 | 24.951 | −6.618 | 1.00 | 73.98 | C |
| ATOM | 8638 | N | GLN | C | 180 | 12.128 | 23.315 | −5.085 | 1.00 | 62.75 | C |
| ATOM | 8639 | CA | GLN | C | 180 | 12.911 | 22.343 | −5.854 | 1.00 | 62.75 | C |
| ATOM | 8640 | CB | GLN | C | 180 | 13.724 | 21.466 | −4.911 | 1.00 | 74.34 | C |
| ATOM | 8641 | CG | GLN | C | 180 | 14.584 | 22.217 | −3.948 | 1.00 | 65.97 | C |
| ATOM | 8642 | CD | GLN | C | 180 | 15.570 | 23.105 | −4.644 | 1.00 | 65.97 | C |
| ATOM | 8643 | OE1 | GLN | C | 180 | 15.372 | 24.314 | −4.734 | 1.00 | 65.97 | C |
| ATOM | 8644 | NE2 | GLN | C | 180 | 16.645 | 22.509 | −5.154 | 1.00 | 65.97 | C |
| ATOM | 8645 | C | GLN | C | 180 | 12.089 | 21.409 | −6.749 | 1.00 | 62.75 | C |
| ATOM | 8646 | O | GLN | C | 180 | 11.254 | 20.645 | −6.266 | 1.00 | 62.75 | C |
| ATOM | 8647 | N | LEU | C | 181 | 12.336 | 21.454 | −8.051 | 1.00 | 66.00 | C |
| ATOM | 8648 | CA | LEU | C | 181 | 11.630 | 20.567 | −8.961 | 1.00 | 66.00 | C |
| ATOM | 8649 | CB | LEU | C | 181 | 11.942 | 20.930 | −10.410 | 1.00 | 87.35 | C |
| ATOM | 8650 | CG | LEU | C | 181 | 11.336 | 19.972 | −11.435 | 1.00 | 74.78 | C |
| ATOM | 8651 | CD1 | LEU | C | 181 | 9.815 | 19.996 | −11.314 | 1.00 | 74.78 | C |
| ATOM | 8652 | CD2 | LEU | C | 181 | 11.774 | 20.358 | −12.837 | 1.00 | 74.78 | C |
| ATOM | 8653 | C | LEU | C | 181 | 12.098 | 19.137 | −8.673 | 1.00 | 66.00 | C |
| ATOM | 8654 | O | LEU | C | 181 | 13.302 | 18.870 | −8.590 | 1.00 | 66.00 | C |
| ATOM | 8655 | N | VAL | C | 182 | 11.149 | 18.222 | −8.509 | 1.00 | 55.70 | C |
| ATOM | 8656 | CA | VAL | C | 182 | 11.492 | 16.836 | −8.229 | 1.00 | 55.70 | C |
| ATOM | 8657 | CB | VAL | C | 182 | 10.722 | 16.317 | −7.010 | 1.00 | 77.72 | C |
| ATOM | 8658 | CG1 | VAL | C | 182 | 11.061 | 14.852 | −6.759 | 1.00 | 56.20 | C |
| ATOM | 8659 | CG2 | VAL | C | 182 | 11.066 | 17.157 | −5.800 | 1.00 | 56.20 | C |
| ATOM | 8660 | C | VAL | C | 182 | 11.176 | 15.937 | −9.415 | 1.00 | 55.70 | C |
| ATOM | 8661 | O | VAL | C | 182 | 11.937 | 15.020 | −9.737 | 1.00 | 55.70 | C |
| ATOM | 8662 | N | THR | C | 183 | 10.051 | 16.211 | −10.066 | 1.00 | 55.09 | C |
| ATOM | 8663 | CA | THR | C | 183 | 9.627 | 15.411 | −11.200 | 1.00 | 55.09 | C |
| ATOM | 8664 | CB | THR | C | 183 | 8.741 | 14.231 | −10.743 | 1.00 | 81.24 | C |
| ATOM | 8665 | OG1 | THR | C | 183 | 9.518 | 13.325 | −9.956 | 1.00 | 67.83 | C |
| ATOM | 8666 | CG2 | THR | C | 183 | 8.155 | 13.495 | −11.941 | 1.00 | 67.83 | C |
| ATOM | 8667 | C | THR | C | 183 | 8.816 | 16.192 | −12.214 | 1.00 | 55.09 | C |
| ATOM | 8668 | O | THR | C | 183 | 7.965 | 17.012 | −11.856 | 1.00 | 55.09 | C |
| ATOM | 8669 | N | LYS | C | 184 | 9.087 | 15.936 | −13.488 | 1.00 | 70.41 | C |
| ATOM | 8670 | CA | LYS | C | 184 | 8.317 | 16.564 | −14.544 | 1.00 | 70.41 | C |
| ATOM | 8671 | CB | LYS | C | 184 | 9.135 | 17.558 | −15.362 | 1.00 | 83.88 | C |
| ATOM | 8672 | CG | LYS | C | 184 | 8.296 | 18.160 | −16.484 | 1.00 | 75.72 | C |
| ATOM | 8673 | CD | LYS | C | 184 | 9.087 | 19.070 | −17.387 | 1.00 | 75.72 | C |
| ATOM | 8674 | CE | LYS | C | 184 | 8.220 | 19.585 | −18.523 | 1.00 | 75.72 | C |
| ATOM | 8675 | NZ | LYS | C | 184 | 9.015 | 20.380 | −19.506 | 1.00 | 75.72 | C |
| ATOM | 8676 | C | LYS | C | 184 | 7.849 | 15.445 | −15.453 | 1.00 | 70.41 | C |
| ATOM | 8677 | O | LYS | C | 184 | 8.621 | 14.556 | −15.804 | 1.00 | 70.41 | C |
| ATOM | 8678 | N | ILE | C | 185 | 6.576 | 15.484 | −15.815 | 1.00 | 67.64 | C |
| ATOM | 8679 | CA | ILE | C | 185 | 6.010 | 14.475 | −16.685 | 1.00 | 67.64 | C |
| ATOM | 8680 | CB | ILE | C | 185 | 5.136 | 13.475 | −15.890 | 1.00 | 87.32 | C |
| ATOM | 8681 | CG2 | ILE | C | 185 | 4.313 | 12.613 | −16.841 | 1.00 | 56.24 | C |
| ATOM | 8682 | CG1 | ILE | C | 185 | 6.025 | 12.584 | −15.022 | 1.00 | 56.24 | C |
| ATOM | 8683 | CD1 | ILE | C | 185 | 5.248 | 11.707 | −14.071 | 1.00 | 56.24 | C |
| ATOM | 8684 | C | ILE | C | 185 | 5.150 | 15.174 | −17.712 | 1.00 | 67.64 | C |
| ATOM | 8685 | O | ILE | C | 185 | 4.432 | 16.123 | −17.387 | 1.00 | 67.64 | C |
| ATOM | 8686 | N | SER | C | 186 | 5.236 | 14.722 | −18.959 | 1.00 | 73.84 | C |
| ATOM | 8687 | CA | SER | C | 186 | 4.428 | 15.311 | −20.018 | 1.00 | 73.84 | C |
| ATOM | 8688 | CB | SER | C | 186 | 5.265 | 16.285 | −20.851 | 1.00 | 101.50 | C |
| ATOM | 8689 | OG | SER | C | 186 | 6.534 | 15.739 | −21.146 | 1.00 | 90.61 | C |
| ATOM | 8690 | C | SER | C | 186 | 3.815 | 14.225 | −20.888 | 1.00 | 73.84 | C |
| ATOM | 8691 | O | SER | C | 186 | 4.353 | 13.119 | −20.997 | 1.00 | 73.84 | C |
| ATOM | 8692 | N | TYR | C | 187 | 2.666 | 14.546 | −21.472 | 1.00 | 71.24 | C |
| ATOM | 8693 | CA | TYR | C | 187 | 1.943 | 13.617 | −22.322 | 1.00 | 71.24 | C |
| ATOM | 8694 | CB | TYR | C | 187 | 0.494 | 14.068 | −22.473 | 1.00 | 132.32 | C |
| ATOM | 8695 | CG | TYR | C | 187 | −0.354 | 13.074 | −23.218 | 1.00 | 86.53 | C |
| ATOM | 8696 | CD1 | TYR | C | 187 | −0.686 | 11.850 | −22.642 | 1.00 | 86.53 | C |
| ATOM | 8697 | CE1 | TYR | C | 187 | −1.454 | 10.918 | −23.327 | 1.00 | 86.53 | C |
| ATOM | 8698 | CD2 | TYR | C | 187 | −0.811 | 13.343 | −24.505 | 1.00 | 86.53 | C |
| ATOM | 8699 | CE2 | TYR | C | 187 | −1.579 | 12.417 | −25.202 | 1.00 | 86.53 | C |
| ATOM | 8700 | CZ | TYR | C | 187 | −1.898 | 11.208 | −24.607 | 1.00 | 86.53 | C |
| ATOM | 8701 | OH | TYR | C | 187 | −2.667 | 10.287 | −25.278 | 1.00 | 86.53 | C |
| ATOM | 8702 | C | TYR | C | 187 | 2.573 | 13.491 | −23.711 | 1.00 | 71.24 | C |
| ATOM | 8703 | O | TYR | C | 187 | 2.670 | 14.465 | −24.455 | 1.00 | 71.24 | C |
| ATOM | 8704 | N | ASN | C | 188 | 2.995 | 12.278 | −24.047 | 1.00 | 114.91 | C |
| ATOM | 8705 | CA | ASN | C | 188 | 3.610 | 11.994 | −25.334 | 1.00 | 114.91 | C |
| ATOM | 8706 | CB | ASN | C | 188 | 4.529 | 10.787 | −25.193 | 1.00 | 200.84 | C |
| ATOM | 8707 | CG | ASN | C | 188 | 5.243 | 10.458 | −26.471 | 1.00 | 82.71 | C |
| ATOM | 8708 | OD1 | ASN | C | 188 | 4.620 | 10.300 | −27.518 | 1.00 | 82.71 | C |
| ATOM | 8709 | ND2 | ASN | C | 188 | 6.559 | 10.347 | −26.398 | 1.00 | 82.71 | C |
| ATOM | 8710 | C | ASN | C | 188 | 2.512 | 11.694 | −26.358 | 1.00 | 114.91 | C |
| ATOM | 8711 | O | ASN | C | 188 | 1.832 | 10.672 | −26.263 | 1.00 | 114.91 | C |
| ATOM | 8712 | N | HIS | C | 189 | 2.342 | 12.575 | −27.339 | 1.00 | 98.57 | C |
| ATOM | 8713 | CA | HIS | C | 189 | 1.303 | 12.391 | −28.350 | 1.00 | 98.57 | C |
| ATOM | 8714 | CB | HIS | C | 189 | 1.139 | 13.677 | −29.143 | 1.00 | 135.71 | C |
| ATOM | 8715 | CG | HIS | C | 189 | 0.468 | 14.756 | −28.366 | 1.00 | 96.25 | C |
| ATOM | 8716 | CD2 | HIS | C | 189 | 0.973 | 15.813 | −27.691 | 1.00 | 96.25 | C |
| ATOM | 8717 | ND1 | HIS | C | 189 | −0.892 | 14.763 | −28.136 | 1.00 | 96.25 | C |
| ATOM | 8718 | CE1 | HIS | C | 189 | −1.195 | 15.779 | −27.349 | 1.00 | 96.25 | C |
| ATOM | 8719 | NE2 | HIS | C | 189 | −0.081 | 16.432 | −27.064 | 1.00 | 96.25 | C |
| ATOM | 8720 | C | HIS | C | 189 | 1.510 | 11.218 | −29.293 | 1.00 | 98.57 | C |
| ATOM | 8721 | O | HIS | C | 189 | 0.549 | 10.647 | −29.812 | 1.00 | 98.57 | C |
| ATOM | 8722 | N | GLU | C | 190 | 2.765 | 10.860 | −29.509 | 1.00 | 117.70 | C |
| ATOM | 8723 | CA | GLU | C | 190 | 3.092 | 9.750 | −30.380 | 1.00 | 117.70 | C |
| ATOM | 8724 | CB | GLU | C | 190 | 4.594 | 9.754 | −30.656 | 1.00 | 162.77 | C |
| ATOM | 8725 | CG | GLU | C | 190 | 5.017 | 9.054 | −31.928 | 1.00 | 137.19 | C |
| ATOM | 8726 | CD | GLU | C | 190 | 6.505 | 9.204 | −32.187 | 1.00 | 137.19 | C |
| ATOM | 8727 | OE1 | GLU | C | 190 | 7.307 | 8.679 | −31.385 | 1.00 | 137.19 | C |
| ATOM | 8728 | OE2 | GLU | C | 190 | 6.878 | 9.853 | −33.187 | 1.00 | 137.19 | C |
| ATOM | 8729 | C | GLU | C | 190 | 2.685 | 8.464 | −29.665 | 1.00 | 117.70 | C |
| ATOM | 8730 | O | GLU | C | 190 | 1.899 | 7.672 | −30.184 | 1.00 | 117.70 | C |
| ATOM | 8731 | N | THR | C | 191 | 3.204 | 8.288 | −28.453 | 1.00 | 103.47 | C |
| ATOM | 8732 | CA | THR | C | 191 | 2.942 | 7.106 | −27.636 | 1.00 | 103.47 | C |
| ATOM | 8733 | CB | THR | C | 191 | 3.999 | 6.973 | −26.536 | 1.00 | 89.25 | C |
| ATOM | 8734 | OG1 | THR | C | 191 | 5.289 | 6.810 | −27.131 | 1.00 | 89.25 | C |
| ATOM | 8735 | CG2 | THR | C | 191 | 3.706 | 5.782 | −25.663 | 1.00 | 89.25 | C |
| ATOM | 8736 | C | THR | C | 191 | 1.568 | 7.056 | −26.973 | 1.00 | 103.47 | C |
| ATOM | 8737 | O | THR | C | 191 | 1.039 | 5.974 | −26.724 | 1.00 | 103.47 | C |
| ATOM | 8738 | N | ARG | C | 192 | 1.005 | 8.221 | −26.670 | 1.00 | 88.03 | C |
| ATOM | 8739 | CA | ARG | C | 192 | −0.306 | 8.312 | −26.029 | 1.00 | 88.03 | C |
| ATOM | 8740 | CB | ARG | C | 192 | −1.311 | 7.395 | −26.735 | 1.00 | 155.17 | C |
| ATOM | 8741 | CG | ARG | C | 192 | −1.617 | 7.828 | −28.152 | 1.00 | 155.17 | C |
| ATOM | 8742 | CD | ARG | C | 192 | −2.140 | 9.254 | −28.168 | 1.00 | 155.17 | C |
| ATOM | 8743 | NE | ARG | C | 192 | −2.269 | 9.777 | −29.524 | 1.00 | 155.17 | C |
| ATOM | 8744 | CZ | ARG | C | 192 | −3.108 | 9.298 | −30.437 | 1.00 | 155.17 | C |
| ATOM | 8745 | NH1 | ARG | C | 192 | −3.905 | 8.278 | −30.144 | 1.00 | 155.17 | C |
| ATOM | 8746 | NH2 | ARG | C | 192 | −3.149 | 9.839 | −31.648 | 1.00 | 155.17 | C |
| ATOM | 8747 | C | ARG | C | 192 | −0.308 | 8.021 | −24.526 | 1.00 | 88.03 | C |
| ATOM | 8748 | O | ARG | C | 192 | −1.298 | 7.533 | −23.981 | 1.00 | 88.03 | C |
| ATOM | 8749 | N | HIS | C | 193 | 0.805 | 8.317 | −23.865 | 1.00 | 106.35 | C |
| ATOM | 8750 | CA | HIS | C | 193 | 0.921 | 8.128 | −22.425 | 1.00 | 106.35 | C |
| ATOM | 8751 | CB | HIS | C | 193 | 1.252 | 6.664 | −22.089 | 1.00 | 119.25 | C |
| ATOM | 8752 | CG | HIS | C | 193 | 2.654 | 6.249 | −22.414 | 1.00 | 119.25 | C |
| ATOM | 8753 | CD2 | HIS | C | 193 | 3.775 | 6.971 | −22.653 | 1.00 | 119.25 | C |
| ATOM | 8754 | ND1 | HIS | C | 193 | 3.037 | 4.926 | −22.458 | 1.00 | 119.25 | C |
| ATOM | 8755 | CE1 | HIS | C | 193 | 4.332 | 4.850 | −22.708 | 1.00 | 119.25 | C |
| ATOM | 8756 | NE2 | HIS | C | 193 | 4.803 | 6.077 | −22.830 | 1.00 | 119.25 | C |
| ATOM | 8757 | C | HIS | C | 193 | 1.996 | 9.091 | −21.926 | 1.00 | 106.35 | C |
| ATOM | 8758 | O | HIS | C | 193 | 2.699 | 9.694 | −22.731 | 1.00 | 106.35 | C |
| ATOM | 8759 | N | GLY | C | 194 | 2.134 | 9.244 | −20.613 | 1.00 | 72.04 | C |
| ATOM | 8760 | CA | GLY | C | 194 | 3.123 | 10.180 | −20.106 | 1.00 | 72.04 | C |
| ATOM | 8761 | C | GLY | C | 194 | 4.554 | 9.680 | −20.084 | 1.00 | 72.04 | C |
| ATOM | 8762 | O | GLY | C | 194 | 4.797 | 8.478 | −20.160 | 1.00 | 72.04 | C |
| ATOM | 8763 | N | ASN | C | 195 | 5.502 | 10.610 | −19.990 | 1.00 | 77.90 | C |
| ATOM | 8764 | CA | ASN | C | 195 | 6.924 | 10.276 | −19.917 | 1.00 | 77.90 | C |
| ATOM | 8765 | CB | ASN | C | 195 | 7.639 | 10.538 | −21.241 | 1.00 | 140.84 | C |
| ATOM | 8766 | CG | ASN | C | 195 | 7.226 | 9.589 | −22.325 | 1.00 | 140.84 | C |
| ATOM | 8767 | OD1 | ASN | C | 195 | 7.132 | 8.380 | −22.107 | 1.00 | 140.84 | C |
| ATOM | 8768 | ND2 | ASN | C | 195 | 6.993 | 10.125 | −23.517 | 1.00 | 140.84 | C |
| ATOM | 8769 | C | ASN | C | 195 | 7.576 | 11.161 | −18.873 | 1.00 | 77.90 | C |
| ATOM | 8770 | O | ASN | C | 195 | 7.261 | 12.350 | −18.780 | 1.00 | 77.90 | C |
| ATOM | 8771 | N | VAL | C | 196 | 8.485 | 10.594 | −18.089 | 1.00 | 74.71 | C |
| ATOM | 8772 | CA | VAL | C | 196 | 9.175 | 11.391 | −17.090 | 1.00 | 74.71 | C |
| ATOM | 8773 | CB | VAL | C | 196 | 9.816 | 10.524 | −15.998 | 1.00 | 104.19 | C |
| ATOM | 8774 | CG1 | VAL | C | 196 | 10.596 | 11.407 | −15.030 | 1.00 | 104.19 | C |
| ATOM | 8775 | CG2 | VAL | C | 196 | 8.742 | 9.744 | −15.263 | 1.00 | 104.19 | C |
| ATOM | 8776 | C | VAL | C | 196 | 10.279 | 12.131 | −17.820 | 1.00 | 74.71 | C |
| ATOM | 8777 | O | VAL | C | 196 | 11.183 | 11.503 | −18.370 | 1.00 | 74.71 | C |
| ATOM | 8778 | N | ASN | C | 197 | 10.203 | 13.459 | −17.834 | 1.00 | 87.35 | C |
| ATOM | 8779 | CA | ASN | C | 197 | 11.213 | 14.269 | −18.507 | 1.00 | 87.35 | C |
| ATOM | 8780 | CB | ASN | C | 197 | 10.596 | 15.549 | −19.065 | 1.00 | 94.61 | C |
| ATOM | 8781 | CG | ASN | C | 197 | 9.311 | 15.299 | −19.814 | 1.00 | 94.61 | C |
| ATOM | 8782 | OD1 | ASN | C | 197 | 9.230 | 14.399 | −20.650 | 1.00 | 94.61 | C |
| ATOM | 8783 | ND2 | ASN | C | 197 | 8.294 | 16.104 | −19.526 | 1.00 | 94.61 | C |
| ATOM | 8784 | C | ASN | C | 197 | 12.323 | 14.652 | −17.548 | 1.00 | 87.35 | C |
| ATOM | 8785 | O | ASN | C | 197 | 13.479 | 14.783 | −17.941 | 1.00 | 87.35 | C |
| ATOM | 8786 | N | PHE | C | 198 | 11.964 | 14.832 | −16.285 | 1.00 | 82.18 | C |
| ATOM | 8787 | CA | PHE | C | 198 | 12.934 | 15.227 | −15.286 | 1.00 | 82.18 | C |
| ATOM | 8788 | CB | PHE | C | 198 | 12.832 | 16.732 | −15.046 | 1.00 | 79.50 | C |
| ATOM | 8789 | CG | PHE | C | 198 | 13.827 | 17.248 | −14.063 | 1.00 | 79.50 | C |
| ATOM | 8790 | CD1 | PHE | C | 198 | 15.068 | 17.694 | −14.485 | 1.00 | 79.50 | C |
| ATOM | 8791 | CD2 | PHE | C | 198 | 13.544 | 17.240 | −12.706 | 1.00 | 79.50 | C |
| ATOM | 8792 | CE1 | PHE | C | 198 | 16.017 | 18.124 | −13.567 | 1.00 | 79.50 | C |
| ATOM | 8793 | CE2 | PHE | C | 198 | 14.485 | 17.666 | −11.779 | 1.00 | 79.50 | C |
| ATOM | 8794 | CZ | PHE | C | 198 | 15.726 | 18.110 | −12.211 | 1.00 | 79.50 | C |
| ATOM | 8795 | C | PHE | C | 198 | 12.746 | 14.506 | −13.961 | 1.00 | 82.18 | C |
| ATOM | 8796 | O | PHE | C | 198 | 11.626 | 14.205 | −13.552 | 1.00 | 82.18 | C |
| ATOM | 8797 | N | ARG | C | 199 | 13.860 | 14.240 | −13.294 | 1.00 | 76.37 | C |
| ATOM | 8798 | CA | ARG | C | 199 | 13.833 | 13.598 | −11.996 | 1.00 | 76.37 | C |
| ATOM | 8799 | CB | ARG | C | 199 | 13.894 | 12.082 | −12.116 | 1.00 | 89.98 | C |
| ATOM | 8800 | CG | ARG | C | 199 | 13.909 | 11.419 | −10.754 | 1.00 | 89.98 | C |
| ATOM | 8801 | CD | ARG | C | 199 | 12.535 | 11.462 | −10.122 | 1.00 | 89.98 | C |
| ATOM | 8802 | NE | ARG | C | 199 | 11.843 | 10.198 | −10.342 | 1.00 | 89.98 | C |
| ATOM | 8803 | CZ | ARG | C | 199 | 10.544 | 10.078 | −10.594 | 1.00 | 89.98 | C |
| ATOM | 8804 | NH1 | ARG | C | 199 | 9.774 | 11.153 | −10.667 | 1.00 | 89.98 | C |
| ATOM | 8805 | NH2 | ARG | C | 199 | 10.014 | 8.876 | −10.772 | 1.00 | 89.98 | C |
| ATOM | 8806 | C | ARG | C | 199 | 15.037 | 14.072 | −11.205 | 1.00 | 76.37 | C |
| ATOM | 8807 | O | ARG | C | 199 | 16.171 | 13.832 | −11.599 | 1.00 | 76.37 | C |
| ATOM | 8808 | N | ALA | C | 200 | 14.792 | 14.749 | −10.091 | 1.00 | 75.19 | C |
| ATOM | 8809 | CA | ALA | C | 200 | 15.883 | 15.242 | −9.269 | 1.00 | 75.19 | C |
| ATOM | 8810 | CB | ALA | C | 200 | 15.330 | 15.941 | −8.031 | 1.00 | 63.11 | C |
| ATOM | 8811 | C | ALA | C | 200 | 16.803 | 14.088 | −8.872 | 1.00 | 75.19 | C |
| ATOM | 8812 | O | ALA | C | 200 | 16.350 | 13.001 | −8.516 | 1.00 | 75.19 | C |
| ATOM | 8813 | N | ASP | C | 201 | 18.102 | 14.338 | −8.949 | 1.00 | 96.72 | C |
| ATOM | 8814 | CA | ASP | C | 201 | 19.112 | 13.347 | −8.615 | 1.00 | 96.72 | C |
| ATOM | 8815 | CB | ASP | C | 201 | 20.499 | 13.925 | −8.887 | 1.00 | 120.74 | C |
| ATOM | 8816 | CG | ASP | C | 201 | 20.681 | 15.300 | −8.273 | 1.00 | 120.74 | C |
| ATOM | 8817 | OD1 | ASP | C | 201 | 20.015 | 16.249 | −8.741 | 1.00 | 120.74 | C |
| ATOM | 8818 | OD2 | ASP | C | 201 | 21.480 | 15.429 | −7.321 | 1.00 | 120.74 | C |
| ATOM | 8819 | C | ASP | C | 201 | 19.053 | 12.857 | −7.172 | 1.00 | 96.72 | C |
| ATOM | 8820 | O | ASP | C | 201 | 19.431 | 11.721 | −6.890 | 1.00 | 96.72 | C |
| ATOM | 8821 | N | TRP | C | 202 | 18.582 | 13.703 | −6.260 | 1.00 | 102.79 | C |
| ATOM | 8822 | CA | TRP | C | 202 | 18.515 | 13.329 | −4.843 | 1.00 | 102.79 | C |
| ATOM | 8823 | CB | TRP | C | 202 | 18.651 | 14.570 | −3.954 | 1.00 | 83.13 | C |
| ATOM | 8824 | CG | TRP | C | 202 | 17.677 | 15.677 | −4.256 | 1.00 | 83.13 | C |
| ATOM | 8825 | CD2 | TRP | C | 202 | 16.362 | 15.839 | −3.712 | 1.00 | 83.13 | C |
| ATOM | 8826 | CE2 | TRP | C | 202 | 15.828 | 17.031 | −4.255 | 1.00 | 83.13 | C |
| ATOM | 8827 | CE3 | TRP | C | 202 | 15.583 | 15.094 | −2.819 | 1.00 | 83.13 | C |
| ATOM | 8828 | CD1 | TRP | C | 202 | 17.879 | 16.743 | −5.086 | 1.00 | 83.13 | C |
| ATOM | 8829 | NE1 | TRP | C | 202 | 16.774 | 17.563 | −5.088 | 1.00 | 83.13 | C |
| ATOM | 8830 | CZ2 | TRP | C | 202 | 14.552 | 17.494 | −3.933 | 1.00 | 83.13 | C |
| ATOM | 8831 | CZ3 | TRP | C | 202 | 14.316 | 15.555 | −2.498 | 1.00 | 83.13 | C |
| ATOM | 8832 | CH2 | TRP | C | 202 | 13.813 | 16.747 | −3.055 | 1.00 | 83.13 | C |
| ATOM | 8833 | C | TRP | C | 202 | 17.271 | 12.564 | −4.401 | 1.00 | 102.79 | C |
| ATOM | 8834 | O | TRP | C | 202 | 17.208 | 12.077 | −3.271 | 1.00 | 102.79 | C |
| ATOM | 8835 | N | PHE | C | 203 | 16.294 | 12.443 | −5.288 | 1.00 | 76.28 | C |
| ATOM | 8836 | CA | PHE | C | 203 | 15.051 | 11.772 | −4.948 | 1.00 | 76.28 | C |
| ATOM | 8837 | CB | PHE | C | 203 | 13.879 | 12.579 | −5.506 | 1.00 | 65.61 | C |
| ATOM | 8838 | CG | PHE | C | 203 | 12.538 | 12.095 | −5.055 | 1.00 | 65.61 | C |
| ATOM | 8839 | CD1 | PHE | C | 203 | 12.136 | 12.258 | −3.731 | 1.00 | 65.61 | C |
| ATOM | 8840 | CD2 | PHE | C | 203 | 11.667 | 11.490 | −5.954 | 1.00 | 65.61 | C |
| ATOM | 8841 | CE1 | PHE | C | 203 | 10.879 | 11.826 | −3.308 | 1.00 | 65.61 | C |
| ATOM | 8842 | CE2 | PHE | C | 203 | 10.407 | 11.052 | −5.543 | 1.00 | 65.61 | C |
| ATOM | 8843 | CZ | PHE | C | 203 | 10.009 | 11.221 | −4.216 | 1.00 | 65.61 | C |
| ATOM | 8844 | C | PHE | C | 203 | 14.957 | 10.342 | −5.451 | 1.00 | 76.28 | C |
| ATOM | 8845 | O | PHE | C | 203 | 14.768 | 10.118 | −6.644 | 1.00 | 76.28 | C |
| ATOM | 8846 | N | ASN | C | 204 | 15.101 | 9.376 | −4.548 | 1.00 | 73.86 | C |
| ATOM | 8847 | CA | ASN | C | 204 | 14.977 | 7.964 | −4.914 | 1.00 | 73.86 | C |
| ATOM | 8848 | CB | ASN | C | 204 | 15.886 | 7.081 | −4.039 | 1.00 | 111.25 | C |
| ATOM | 8849 | CG | ASN | C | 204 | 15.590 | 5.593 | −4.200 | 1.00 | 111.25 | C |
| ATOM | 8850 | OD1 | ASN | C | 204 | 14.511 | 5.137 | −3.835 | 1.00 | 111.25 | C |
| ATOM | 8851 | ND2 | ASN | C | 204 | 16.524 | 4.840 | −4.775 | 1.00 | 111.25 | C |
| ATOM | 8852 | C | ASN | C | 204 | 13.510 | 7.654 | −4.644 | 1.00 | 73.86 | C |
| ATOM | 8853 | O | ASN | C | 204 | 13.117 | 7.436 | −3.501 | 1.00 | 73.86 | C |
| ATOM | 8854 | N | ILE | C | 205 | 12.697 | 7.655 | −5.694 | 1.00 | 67.17 | C |
| ATOM | 8855 | CA | ILE | C | 205 | 11.263 | 7.411 | −5.544 | 1.00 | 67.17 | C |
| ATOM | 8856 | CB | ILE | C | 205 | 10.554 | 7.441 | −6.905 | 1.00 | 52.23 | C |
| ATOM | 8857 | CG2 | ILE | C | 205 | 10.703 | 6.091 | −7.598 | 1.00 | 52.23 | C |
| ATOM | 8858 | CG1 | ILE | C | 205 | 9.075 | 7.764 | −6.703 | 1.00 | 52.23 | C |
| ATOM | 8859 | CD1 | ILE | C | 205 | 8.380 | 8.276 | −7.958 | 1.00 | 52.23 | C |
| ATOM | 8860 | C | ILE | C | 205 | 10.905 | 6.105 | −4.831 | 1.00 | 67.17 | C |
| ATOM | 8861 | O | ILE | C | 205 | 9.783 | 5.941 | −4.359 | 1.00 | 67.17 | C |
| ATOM | 8862 | N | SER | C | 206 | 11.855 | 5.179 | −4.754 | 1.00 | 83.68 | C |
| ATOM | 8863 | CA | SER | C | 206 | 11.612 | 3.910 | −4.080 | 1.00 | 83.68 | C |
| ATOM | 8864 | CB | SER | C | 206 | 12.687 | 2.896 | −4.460 | 1.00 | 89.38 | C |
| ATOM | 8865 | OG | SER | C | 206 | 12.606 | 2.590 | −5.837 | 1.00 | 89.38 | C |
| ATOM | 8866 | C | SER | C | 206 | 11.602 | 4.105 | −2.569 | 1.00 | 83.68 | C |
| ATOM | 8867 | O | SER | C | 206 | 11.046 | 3.296 | −1.831 | 1.00 | 83.68 | C |
| ATOM | 8868 | N | ARG | C | 207 | 12.224 | 5.185 | −2.111 | 1.00 | 58.41 | C |
| ATOM | 8869 | CA | ARG | C | 207 | 12.274 | 5.475 | −0.692 | 1.00 | 58.41 | C |
| ATOM | 8870 | CB | ARG | C | 207 | 13.608 | 6.127 | −0.326 | 1.00 | 83.72 | C |
| ATOM | 8871 | CG | ARG | C | 207 | 14.769 | 5.152 | −0.305 | 1.00 | 83.72 | C |
| ATOM | 8872 | CD | ARG | C | 207 | 15.828 | 5.578 | 0.700 | 1.00 | 83.72 | C |
| ATOM | 8873 | NE | ARG | C | 207 | 16.640 | 6.701 | 0.238 | 1.00 | 83.72 | C |
| ATOM | 8874 | CZ | ARG | C | 207 | 17.263 | 7.552 | 1.051 | 1.00 | 83.72 | C |
| ATOM | 8875 | NH1 | ARG | C | 207 | 17.159 | 7.406 | 2.370 | 1.00 | 83.72 | C |
| ATOM | 8876 | NH2 | ARG | C | 207 | 17.992 | 8.544 | 0.548 | 1.00 | 83.72 | C |
| ATOM | 8877 | C | ARG | C | 207 | 11.118 | 6.372 | −0.265 | 1.00 | 58.41 | C |
| ATOM | 8878 | O | ARG | C | 207 | 11.103 | 6.875 | 0.857 | 1.00 | 58.41 | C |
| ATOM | 8879 | N | SER | C | 208 | 10.152 | 6.577 | −1.158 | 1.00 | 60.28 | C |
| ATOM | 8880 | CA | SER | C | 208 | 8.997 | 7.416 | −0.848 | 1.00 | 60.28 | C |
| ATOM | 8881 | CB | SER | C | 208 | 9.033 | 8.717 | −1.644 | 1.00 | 86.74 | C |
| ATOM | 8882 | OG | SER | C | 208 | 7.956 | 9.549 | −1.255 | 1.00 | 85.74 | C |
| ATOM | 8883 | C | SER | C | 208 | 7.710 | 6.670 | −1.161 | 1.00 | 60.28 | C |
| ATOM | 8884 | O | SER | C | 208 | 6.999 | 6.989 | −2.122 | 1.00 | 60.28 | C |
| ATOM | 8885 | N | PRO | C | 209 | 7.394 | 5.662 | −0.336 | 1.00 | 60.44 | C |
| ATOM | 8886 | CD | PRO | C | 209 | 8.186 | 5.352 | 0.870 | 1.00 | 55.08 | C |
| ATOM | 8887 | CA | PRO | C | 209 | 6.223 | 4.785 | −0.414 | 1.00 | 60.44 | C |
| ATOM | 8888 | CB | PRO | C | 209 | 6.236 | 4.077 | 0.937 | 1.00 | 55.08 | C |
| ATOM | 8889 | CG | PRO | C | 209 | 7.681 | 3.979 | 1.241 | 1.00 | 55.08 | C |
| ATOM | 8890 | C | PRO | C | 209 | 4.911 | 5.508 | −0.636 | 1.00 | 60.44 | C |
| ATOM | 8891 | O | PRO | C | 209 | 3.982 | 4.951 | −1.223 | 1.00 | 60.44 | C |
| ATOM | 8892 | N | HIS | C | 210 | 4.832 | 6.751 | −0.170 | 1.00 | 61.45 | C |
| ATOM | 8893 | CA | HIS | C | 210 | 3.592 | 7.498 | −0.284 | 1.00 | 61.45 | C |
| ATOM | 8894 | CB | HIS | C | 210 | 3.327 | 8.204 | 1.037 | 1.00 | 63.74 | C |
| ATOM | 8895 | CG | HIS | C | 210 | 3.140 | 7.254 | 2.177 | 1.00 | 63.74 | C |
| ATOM | 8896 | CD2 | HIS | C | 210 | 3.955 | 6.923 | 3.206 | 1.00 | 63.74 | C |
| ATOM | 8897 | ND1 | HIS | C | 210 | 2.020 | 6.460 | 2.301 | 1.00 | 63.74 | C |
| ATOM | 8898 | CE1 | HIS | C | 210 | 2.153 | 5.680 | 3.359 | 1.00 | 63.74 | C |
| ATOM | 8899 | NE2 | HIS | C | 210 | 3.318 | 5.941 | 3.925 | 1.00 | 63.74 | C |
| ATOM | 8900 | C | HIS | C | 210 | 3.471 | 8.446 | −1.455 | 1.00 | 61.45 | C |
| ATOM | 8901 | O | HIS | C | 210 | 2.519 | 9.223 | −1.536 | 1.00 | 61.45 | C |
| ATOM | 8902 | N | THR | C | 211 | 4.442 | 8.391 | −2.359 | 1.00 | 72.09 | C |
| ATOM | 8903 | CA | THR | C | 211 | 4.382 | 9.204 | −3.563 | 1.00 | 72.09 | C |
| ATOM | 8904 | CB | THR | C | 211 | 5.779 | 9.470 | −4.154 | 1.00 | 74.07 | C |
| ATOM | 8905 | OG1 | THR | C | 211 | 6.568 | 10.221 | −3.221 | 1.00 | 74.07 | C |
| ATOM | 8906 | CG2 | THR | C | 211 | 5.652 | 10.254 | −5.448 | 1.00 | 74.07 | C |
| ATOM | 8907 | C | THR | C | 211 | 3.603 | 8.284 | −4.498 | 1.00 | 72.09 | C |
| ATOM | 8908 | O | THR | C | 211 | 3.980 | 7.129 | −4.686 | 1.00 | 72.09 | C |
| ATOM | 8909 | N | PRO | C | 212 | 2.502 | 8.774 | −5.081 | 1.00 | 83.23 | C |
| ATOM | 8910 | CD | PRO | C | 212 | 2.029 | 10.166 | −4.986 | 1.00 | 67.57 | C |
| ATOM | 8911 | CA | PRO | C | 212 | 1.657 | 7.990 | −5.994 | 1.00 | 83.23 | C |
| ATOM | 8912 | CB | PRO | C | 212 | 0.751 | 9.046 | −6.615 | 1.00 | 67.57 | C |
| ATOM | 8913 | CG | PRO | C | 212 | 0.608 | 10.057 | −5.500 | 1.00 | 67.57 | C |
| ATOM | 8914 | C | PRO | C | 212 | 2.408 | 7.178 | −7.052 | 1.00 | 83.23 | C |
| ATOM | 8915 | O | PRO | C | 212 | 3.394 | 7.648 | −7.622 | 1.00 | 83.23 | C |
| ATOM | 8916 | N | GLY | C | 213 | 1.931 | 5.961 | −7.313 | 1.00 | 78.37 | C |
| ATOM | 8917 | CA | GLY | C | 213 | 2.555 | 5.098 | −8.309 | 1.00 | 78.37 | C |
| ATOM | 8918 | C | GLY | C | 213 | 2.063 | 5.380 | −9.718 | 1.00 | 78.37 | C |
| ATOM | 8919 | O | GLY | C | 213 | 1.220 | 6.258 | −9.915 | 1.00 | 78.37 | C |
| ATOM | 8920 | N | ASN | C | 214 | 2.571 | 4.635 | −10.698 | 1.00 | 75.62 | C |
| ATOM | 8921 | CA | ASN | C | 214 | 2.192 | 4.828 | −12.101 | 1.00 | 75.62 | C |
| ATOM | 8922 | CB | ASN | C | 214 | 0.697 | 4.604 | −12.325 | 1.00 | 103.20 | C |
| ATOM | 8923 | CG | ASN | C | 214 | 0.155 | 3.467 | −11.522 | 1.00 | 97.37 | C |
| ATOM | 8924 | OD1 | ASN | C | 214 | 0.909 | 2.640 | −11.016 | 1.00 | 97.37 | C |
| ATOM | 8925 | ND2 | ASN | C | 214 | −1.167 | 3.407 | −11.401 | 1.00 | 97.37 | C |
| ATOM | 8926 | C | ASN | C | 214 | 2.499 | 6.262 | −12.479 | 1.00 | 75.62 | C |
| ATOM | 8927 | O | ASN | C | 214 | 1.643 | 6.967 | −13.026 | 1.00 | 75.62 | C |
| ATOM | 8928 | N | ASP | C | 215 | 3.718 | 6.696 | −12.179 | 1.00 | 89.35 | C |
| ATOM | 8929 | CA | ASP | C | 215 | 4.121 | 8.059 | −12.476 | 1.00 | 89.35 | C |
| ATOM | 8930 | CB | ASP | C | 215 | 4.398 | 8.210 | −13.964 | 1.00 | 180.94 | C |
| ATOM | 8931 | CG | ASP | C | 215 | 5.432 | 7.229 | −14.451 | 1.00 | 102.32 | C |
| ATOM | 8932 | OD1 | ASP | C | 215 | 6.550 | 7.214 | −13.894 | 1.00 | 102.32 | C |
| ATOM | 8933 | OD2 | ASP | C | 215 | 5.128 | 6.467 | −15.385 | 1.00 | 102.32 | C |
| ATOM | 8934 | C | ASP | C | 215 | 3.017 | 9.010 | −12.050 | 1.00 | 89.35 | C |
| ATOM | 8935 | O | ASP | C | 215 | 2.404 | 9.687 | −12.882 | 1.00 | 89.35 | C |
| ATOM | 8936 | N | TYR | C | 216 | 2.755 | 9.024 | −10.745 | 1.00 | 57.38 | C |
| ATOM | 8937 | CA | TYR | C | 216 | 1.748 | 9.896 | −10.155 | 1.00 | 57.38 | C |
| ATOM | 8938 | CB | TYR | C | 216 | 2.209 | 11.358 | −10.242 | 1.00 | 75.92 | C |
| ATOM | 8939 | CG | TYR | C | 216 | 3.594 | 11.596 | −9.694 | 1.00 | 54.67 | C |
| ATOM | 8940 | CD1 | TYR | C | 216 | 4.714 | 11.081 | −10.340 | 1.00 | 54.67 | C |
| ATOM | 8941 | CE1 | TYR | C | 216 | 5.993 | 11.269 | −9.827 | 1.00 | 54.67 | C |
| ATOM | 8942 | CD2 | TYR | C | 216 | 3.787 | 12.316 | −8.511 | 1.00 | 54.67 | C |
| ATOM | 8943 | CE2 | TYR | C | 216 | 5.060 | 12.516 | −7.987 | 1.00 | 54.67 | C |
| ATOM | 8944 | CZ | TYR | C | 216 | 6.160 | 11.989 | −8.653 | 1.00 | 54.67 | C |
| ATOM | 8945 | OH | TYR | C | 216 | 7.430 | 12.179 | −8.157 | 1.00 | 54.67 | C |
| ATOM | 8946 | C | TYR | C | 216 | 0.356 | 9.769 | −10.773 | 1.00 | 57.38 | C |
| ATOM | 8947 | O | TYR | C | 216 | −0.391 | 10.750 | −10.830 | 1.00 | 57.38 | C |
| ATOM | 8948 | N | ASN | C | 217 | −0.001 | 8.565 | −11.214 | 1.00 | 65.08 | C |
| ATOM | 8949 | CA | ASN | C | 217 | −1.309 | 8.338 | −11.824 | 1.00 | 65.08 | C |
| ATOM | 8950 | CB | ASN | C | 217 | −2.427 | 8.499 | −10.784 | 1.00 | 77.48 | C |
| ATOM | 8951 | CG | ASN | C | 217 | −2.329 | 7.491 | −9.651 | 1.00 | 72.21 | C |
| ATOM | 8952 | OD1 | ASN | C | 217 | −2.309 | 7.863 | −8.477 | 1.00 | 72.21 | C |
| ATOM | 8953 | ND2 | ASN | C | 217 | −2.276 | 6.211 | −9.997 | 1.00 | 72.21 | C |
| ATOM | 8954 | C | ASN | C | 217 | −1.512 | 9.354 | −12.940 | 1.00 | 65.08 | C |
| ATOM | 8955 | O | ASN | C | 217 | −2.627 | 9.842 | −13.160 | 1.00 | 65.08 | C |
| ATOM | 8956 | N | PHE | C | 218 | −0.434 | 9.668 | −13.651 | 1.00 | 69.52 | C |
| ATOM | 8957 | CA | PHE | C | 218 | −0.512 | 10.656 | −14.721 | 1.00 | 69.52 | C |
| ATOM | 8958 | CB | PHE | C | 218 | 0.851 | 10.890 | −15.353 | 1.00 | 89.43 | C |
| ATOM | 8959 | CG | PHE | C | 218 | 0.836 | 11.969 | −16.382 | 1.00 | 85.95 | C |
| ATOM | 8960 | CD1 | PHE | C | 218 | 0.925 | 13.303 | −16.009 | 1.00 | 85.95 | C |
| ATOM | 8961 | CD2 | PHE | C | 218 | 0.665 | 11.661 | −17.722 | 1.00 | 85.95 | C |
| ATOM | 8962 | CE1 | PHE | C | 218 | 0.840 | 14.319 | −16.958 | 1.00 | 85.95 | C |
| ATOM | 8963 | CE2 | PHE | C | 218 | 0.579 | 12.668 | −18.678 | 1.00 | 85.95 | C |
| ATOM | 8964 | CZ | PHE | C | 218 | 0.667 | 13.999 | −18.294 | 1.00 | 85.95 | C |
| ATOM | 8965 | C | PHE | C | 218 | −1.499 | 10.332 | −15.831 | 1.00 | 69.52 | C |
| ATOM | 8966 | O | PHE | C | 218 | −2.339 | 11.164 | −16.178 | 1.00 | 69.52 | C |
| ATOM | 8967 | N | ASN | C | 219 | −1.387 | 9.136 | −16.404 | 1.00 | 69.12 | C |
| ATOM | 8968 | CA | ASN | C | 219 | −2.296 | 8.741 | −17.472 | 1.00 | 69.12 | C |
| ATOM | 8969 | CB | ASN | C | 219 | −2.036 | 7.299 | −17.908 | 1.00 | 88.57 | C |
| ATOM | 8970 | CG | ASN | C | 219 | −0.618 | 7.085 | −18.382 | 1.00 | 80.20 | C |
| ATOM | 8971 | OD1 | ASN | C | 219 | −0.016 | 7.968 | −18.988 | 1.00 | 80.20 | C |
| ATOM | 8972 | ND2 | ASN | C | 219 | −0.077 | 5.903 | −18.121 | 1.00 | 80.20 | C |
| ATOM | 8973 | C | ASN | C | 219 | −3.740 | 8.887 | −17.002 | 1.00 | 69.12 | C |
| ATOM | 8974 | O | ASN | C | 219 | −4.590 | 9.392 | −17.737 | 1.00 | 69.12 | C |
| ATOM | 8975 | N | PHE | C | 220 | −4.016 | 8.458 | −15.772 | 1.00 | 67.46 | C |
| ATOM | 8976 | CA | PHE | C | 220 | −5.365 | 8.564 | −15.235 | 1.00 | 67.46 | C |
| ATOM | 8977 | CB | PHE | C | 220 | −5.426 | 8.034 | −13.802 | 1.00 | 64.94 | C |
| ATOM | 8978 | CG | PHE | C | 220 | −6.699 | 8.400 | −13.078 | 1.00 | 57.92 | C |
| ATOM | 8979 | CD1 | PHE | C | 220 | −7.858 | 7.649 | −13.253 | 1.00 | 57.92 | C |
| ATOM | 8980 | CD2 | PHE | C | 220 | −6.757 | 9.540 | −12.275 | 1.00 | 57.92 | C |
| ATOM | 8981 | CE1 | PHE | C | 220 | −9.060 | 8.029 | −12.640 | 1.00 | 57.92 | C |
| ATOM | 8982 | CE2 | PHE | C | 220 | −7.949 | 9.930 | −11.662 | 1.00 | 57.92 | C |
| ATOM | 8983 | CZ | PHE | C | 220 | −9.103 | 9.173 | −11.845 | 1.00 | 57.92 | C |
| ATOM | 8984 | C | PHE | C | 220 | −5.841 | 10.009 | −15.238 | 1.00 | 67.46 | C |
| ATOM | 8985 | O | PHE | C | 220 | −6.896 | 10.324 | −15.794 | 1.00 | 67.46 | C |
| ATOM | 8986 | N | TRP | C | 221 | −5.061 | 10.884 | −14.607 | 1.00 | 55.74 | C |
| ATOM | 8987 | CA | TRP | C | 221 | −5.430 | 12.291 | −14.518 | 1.00 | 55.74 | C |
| ATOM | 8988 | CB | TRP | C | 221 | −4.484 | 13.021 | −13.558 | 1.00 | 54.66 | C |
| ATOM | 8989 | CG | TRP | C | 221 | −4.734 | 12.684 | −12.116 | 1.00 | 47.81 | C |
| ATOM | 8990 | CD2 | TRP | C | 221 | −5.945 | 12.917 | −11.384 | 1.00 | 47.81 | C |
| ATOM | 8991 | CE2 | TRP | C | 221 | −5.764 | 12.370 | −10.091 | 1.00 | 47.81 | C |
| ATOM | 8992 | CE3 | TRP | C | 221 | −7.169 | 13.529 | −11.697 | 1.00 | 47.81 | C |
| ATOM | 8993 | CD1 | TRP | C | 221 | −3.884 | 12.030 | −11.258 | 1.00 | 47.81 | C |
| ATOM | 8994 | NE1 | TRP | C | 221 | −4.501 | 11.836 | −10.039 | 1.00 | 47.81 | C |
| ATOM | 8995 | CZ2 | TRP | C | 221 | −6.759 | 12.417 | −9.113 | 1.00 | 47.81 | C |
| ATOM | 8996 | CZ3 | TRP | C | 221 | −8.167 | 13.573 | −10.720 | 1.00 | 47.81 | C |
| ATOM | 8997 | CH2 | TRP | C | 221 | −7.952 | 13.019 | −9.443 | 1.00 | 47.81 | C |
| ATOM | 8998 | C | TRP | C | 221 | −5.472 | 13.009 | −15.864 | 1.00 | 55.74 | C |
| ATOM | 8999 | O | TRP | C | 221 | −6.426 | 13.733 | −16.166 | 1.00 | 55.74 | C |
| ATOM | 9000 | N | TYR | C | 222 | −4.442 | 12.814 | −16.676 | 1.00 | 68.50 | C |
| ATOM | 9001 | CA | TYR | C | 222 | −4.415 | 13.466 | −17.976 | 1.00 | 68.50 | C |
| ATOM | 9002 | CB | TYR | C | 222 | −3.273 | 12.927 | −18.830 | 1.00 | 96.07 | C |
| ATOM | 9003 | CG | TYR | C | 222 | −3.370 | 13.410 | −20.255 | 1.00 | 94.64 | C |
| ATOM | 9004 | CD1 | TYR | C | 222 | −2.989 | 14.707 | −20.603 | 1.00 | 94.64 | C |
| ATOM | 9005 | CE1 | TYR | C | 222 | −3.153 | 15.178 | −21.899 | 1.00 | 94.64 | C |
| ATOM | 9006 | CD2 | TYR | C | 222 | −3.915 | 12.595 | −21.246 | 1.00 | 94.64 | C |
| ATOM | 9007 | CE2 | TYR | C | 222 | −4.085 | 13.057 | −22.544 | 1.00 | 94.64 | C |
| ATOM | 9008 | CZ | TYR | C | 222 | −3.705 | 14.345 | −22.862 | 1.00 | 94.64 | C |
| ATOM | 9009 | OH | TYR | C | 222 | −3.894 | 14.797 | −24.142 | 1.00 | 94.64 | C |
| ATOM | 9010 | C | TYR | C | 222 | −5.724 | 13.217 | −18.711 | 1.00 | 68.50 | C |
| ATOM | 9011 | O | TYR | C | 222 | −6.399 | 14.144 | −19.168 | 1.00 | 68.50 | C |
| ATOM | 9012 | N | SER | C | 223 | −6.071 | 11.942 | −18.815 | 1.00 | 81.71 | C |
| ATOM | 9013 | CA | SER | C | 223 | −7.276 | 11.527 | −19.501 | 1.00 | 81.71 | C |
| ATOM | 9014 | CB | SER | C | 223 | −7.346 | 10.007 | −19.518 | 1.00 | 85.37 | C |
| ATOM | 9015 | OG | SER | C | 223 | −8.573 | 9.582 | −20.067 | 1.00 | 83.99 | C |
| ATOM | 9016 | C | SER | C | 223 | −8.553 | 12.095 | −18.898 | 1.00 | 81.71 | C |
| ATOM | 9017 | O | SER | C | 223 | −9.383 | 12.651 | −19.615 | 1.00 | 81.71 | C |
| ATOM | 9018 | N | LEU | C | 224 | −8.711 | 11.957 | −17.585 | 1.00 | 69.80 | C |
| ATOM | 9019 | CA | LEU | C | 224 | −9.911 | 12.447 | −16.917 | 1.00 | 69.80 | C |
| ATOM | 9020 | CB | LEU | C | 224 | −9.959 | 11.944 | −15.475 | 1.00 | 93.80 | C |
| ATOM | 9021 | CG | LEU | C | 224 | −11.188 | 12.409 | −14.696 | 1.00 | 71.04 | C |
| ATOM | 9022 | CD1 | LEU | C | 224 | −12.465 | 12.039 | −15.436 | 1.00 | 71.04 | C |
| ATOM | 9023 | CD2 | LEU | C | 224 | −11.154 | 11.782 | −13.321 | 1.00 | 71.04 | C |
| ATOM | 9024 | C | LEU | C | 224 | −10.007 | 13.968 | −16.942 | 1.00 | 69.80 | C |
| ATOM | 9025 | O | LEU | C | 224 | −11.103 | 14.527 | −16.960 | 1.00 | 69.80 | C |
| ATOM | 9026 | N | MET | C | 225 | −8.859 | 14.637 | −16.938 | 1.00 | 97.64 | C |
| ATOM | 9027 | CA | MET | C | 225 | −8.850 | 16.090 | −16.980 | 1.00 | 97.64 | C |
| ATOM | 9028 | CB | MET | C | 225 | −7.501 | 16.624 | −16.536 | 1.00 | 106.79 | C |
| ATOM | 9029 | CG | MET | C | 225 | −7.250 | 16.397 | −15.075 | 1.00 | 75.51 | C |
| ATOM | 9030 | SD | MET | C | 225 | −5.760 | 17.204 | −14.537 | 1.00 | 75.51 | C |
| ATOM | 9031 | CE | MET | C | 225 | −6.213 | 18.936 | −14.787 | 1.00 | 75.51 | C |
| ATOM | 9032 | C | MET | C | 225 | −9.149 | 16.543 | −18.392 | 1.00 | 97.64 | C |
| ATOM | 9033 | O | MET | C | 225 | −9.813 | 17.558 | −18.606 | 1.00 | 97.64 | C |
| ATOM | 9034 | N | LYS | C | 226 | −8.654 | 15.782 | −19.361 | 1.00 | 89.18 | C |
| ATOM | 9035 | CA | LYS | C | 226 | −8.903 | 16.102 | −20.755 | 1.00 | 89.18 | C |
| ATOM | 9036 | CB | LYS | C | 226 | −8.226 | 15.076 | −21.658 | 1.00 | 136.93 | C |
| ATOM | 9037 | CG | LYS | C | 226 | −8.440 | 15.327 | −23.130 | 1.00 | 88.39 | C |
| ATOM | 9038 | CD | LYS | C | 226 | −7.867 | 14.193 | −23.956 | 1.00 | 88.39 | C |
| ATOM | 9039 | CE | LYS | C | 226 | −8.291 | 14.309 | −25.400 | 1.00 | 88.39 | C |
| ATOM | 9040 | NZ | LYS | C | 226 | −7.761 | 13.175 | −26.188 | 1.00 | 88.39 | C |
| ATOM | 9041 | C | LYS | C | 226 | −10.416 | 16.059 | −20.943 | 1.00 | 89.18 | C |
| ATOM | 9042 | O | LYS | C | 226 | −11.002 | 16.965 | −21.531 | 1.00 | 89.18 | C |
| ATOM | 9043 | N | GLU | C | 227 | −11.043 | 15.008 | −20.418 | 1.00 | 67.66 | C |
| ATOM | 9044 | CA | GLU | C | 227 | −12.491 | 14.843 | −20.512 | 1.00 | 67.66 | C |
| ATOM | 9045 | CB | GLU | C | 227 | −12.934 | 13.563 | −19.799 | 1.00 | 132.41 | C |
| ATOM | 9046 | CG | GLU | C | 227 | −14.417 | 13.262 | −19.957 | 1.00 | 108.21 | C |
| ATOM | 9047 | CD | GLU | C | 227 | −14.922 | 12.206 | −18.983 | 1.00 | 108.21 | C |
| ATOM | 9048 | OE1 | GLU | C | 227 | −14.320 | 11.112 | −18.915 | 1.00 | 108.21 | C |
| ATOM | 9049 | OE2 | GLU | C | 227 | −15.931 | 12.474 | −18.291 | 1.00 | 108.21 | C |
| ATOM | 9050 | C | GLU | C | 227 | −13.187 | 16.035 | −19.869 | 1.00 | 67.66 | C |
| ATOM | 9051 | O | GLU | C | 227 | −14.094 | 16.621 | −20.447 | 1.00 | 67.66 | C |
| ATOM | 9052 | N | THR | C | 228 | −12.748 | 16.394 | −18.669 | 1.00 | 102.40 | C |
| ATOM | 9053 | CA | THR | C | 228 | −13.336 | 17.510 | −17.941 | 1.00 | 102.40 | C |
| ATOM | 9054 | CB | THR | C | 228 | −12.692 | 17.666 | −16.559 | 1.00 | 102.51 | C |
| ATOM | 9055 | OG1 | THR | C | 228 | −12.848 | 16.447 | −15.824 | 1.00 | 88.50 | C |
| ATOM | 9056 | CG2 | THR | C | 228 | −13.349 | 18.813 | −15.793 | 1.00 | 88.50 | C |
| ATOM | 9057 | C | THR | C | 228 | −13.197 | 18.833 | −18.679 | 1.00 | 102.40 | C |
| ATOM | 9058 | O | THR | C | 228 | −14.110 | 19.661 | −18.665 | 1.00 | 102.40 | C |
| ATOM | 9059 | N | LEU | C | 229 | −12.051 | 19.039 | −19.313 | 1.00 | 111.37 | C |
| ATOM | 9060 | CA | LEU | C | 229 | −11.837 | 20.276 | −20.037 | 1.00 | 111.37 | C |
| ATOM | 9061 | CB | LEU | C | 229 | −10.380 | 20.382 | −20.489 | 1.00 | 77.64 | C |
| ATOM | 9062 | CG | LEU | C | 229 | −10.000 | 21.662 | −21.246 | 1.00 | 49.52 | C |
| ATOM | 9063 | CD1 | LEU | C | 229 | −10.586 | 22.894 | −20.570 | 1.00 | 49.52 | C |
| ATOM | 9064 | CD2 | LEU | C | 229 | −8.478 | 21.745 | −21.316 | 1.00 | 49.52 | C |
| ATOM | 9065 | C | LEU | C | 229 | −12.779 | 20.366 | −21.232 | 1.00 | 111.37 | C |
| ATOM | 9066 | O | LEU | C | 229 | −13.330 | 21.430 | −21.511 | 1.00 | 111.37 | C |
| ATOM | 9067 | N | GLU | C | 230 | −12.975 | 19.248 | −21.928 | 1.00 | 82.96 | C |
| ATOM | 9068 | CA | GLU | C | 230 | −13.863 | 19.225 | −23.083 | 1.00 | 82.96 | C |
| ATOM | 9069 | CB | GLU | C | 230 | −13.836 | 17.850 | −23.748 | 1.00 | 142.41 | C |
| ATOM | 9070 | CG | GLU | C | 230 | −12.529 | 17.552 | −24.455 | 1.00 | 106.87 | C |
| ATOM | 9071 | CD | GLU | C | 230 | −12.528 | 16.200 | −25.145 | 1.00 | 106.87 | C |
| ATOM | 9072 | OE1 | GLU | C | 230 | −13.470 | 15.937 | −25.922 | 1.00 | 106.87 | C |
| ATOM | 9073 | OE2 | GLU | C | 230 | −11.586 | 15.405 | −24.919 | 1.00 | 106.87 | C |
| ATOM | 9074 | C | GLU | C | 230 | −15.288 | 19.587 | −22.683 | 1.00 | 82.96 | C |
| ATOM | 9075 | O | GLU | C | 230 | −15.932 | 20.410 | −23.337 | 1.00 | 82.96 | C |
| ATOM | 9076 | N | GLU | C | 231 | −15.776 | 18.981 | −21.607 | 1.00 | 88.07 | C |
| ATOM | 9077 | CA | GLU | C | 231 | −17.126 | 19.257 | −21.118 | 1.00 | 88.07 | C |
| ATOM | 9078 | CB | GLU | C | 231 | −17.438 | 18.390 | −19.896 | 1.00 | 174.77 | C |
| ATOM | 9079 | CG | GLU | C | 231 | −17.265 | 16.899 | −20.122 | 1.00 | 151.01 | C |
| ATOM | 9080 | CD | GLU | C | 231 | −18.277 | 16.330 | −21.094 | 1.00 | 151.01 | C |
| ATOM | 9081 | OE1 | GLU | C | 231 | −18.380 | 16.854 | −22.225 | 1.00 | 151.01 | C |
| ATOM | 9082 | OE2 | GLU | C | 231 | −18.966 | 15.353 | −20.727 | 1.00 | 151.01 | C |
| ATOM | 9083 | C | GLU | C | 231 | −17.246 | 20.729 | −20.734 | 1.00 | 88.07 | C |
| ATOM | 9084 | O | GLU | C | 231 | −18.224 | 21.390 | −21.075 | 1.00 | 88.07 | C |
| ATOM | 9085 | N | ILE | C | 232 | −16.246 | 21.235 | −20.020 | 1.00 | 101.52 | C |
| ATOM | 9086 | CA | ILE | C | 232 | −16.246 | 22.625 | −19.586 | 1.00 | 101.52 | C |
| ATOM | 9087 | CB | ILE | C | 232 | −14.969 | 22.950 | −18.773 | 1.00 | 119.85 | C |
| ATOM | 9088 | CG2 | ILE | C | 232 | −14.803 | 24.453 | −18.627 | 1.00 | 89.90 | C |
| ATOM | 9089 | CG1 | ILE | C | 232 | −15.049 | 22.298 | −17.393 | 1.00 | 89.90 | C |
| ATOM | 9090 | CD1 | ILE | C | 232 | −13.835 | 22.550 | −16.530 | 1.00 | 89.90 | C |
| ATOM | 9091 | C | ILE | C | 232 | −16.342 | 23.595 | −20.761 | 1.00 | 101.52 | C |
| ATOM | 9092 | O | ILE | C | 232 | −16.945 | 24.666 | −20.645 | 1.00 | 101.52 | C |
| ATOM | 9093 | N | ASN | C | 233 | −15.757 | 23.219 | −21.892 | 1.00 | 107.36 | C |
| ATOM | 9094 | CA | ASN | C | 233 | −15.774 | 24.087 | −23.060 | 1.00 | 107.36 | C |
| ATOM | 9095 | CB | ASN | C | 233 | −14.579 | 23.782 | −23.968 | 1.00 | 110.55 | C |
| ATOM | 9096 | CG | ASN | C | 233 | −13.261 | 24.229 | −23.365 | 1.00 | 99.47 | C |
| ATOM | 9097 | OD1 | ASN | C | 233 | −13.204 | 25.221 | −22.638 | 1.00 | 99.47 | C |
| ATOM | 9098 | ND2 | ASN | C | 233 | −12.189 | 23.508 | −23.678 | 1.00 | 99.47 | C |
| ATOM | 9099 | C | ASN | C | 233 | −17.058 | 24.066 | −23.881 | 1.00 | 107.36 | C |
| ATOM | 9100 | O | ASN | C | 233 | −17.247 | 24.923 | −24.744 | 1.00 | 107.36 | C |
| ATOM | 9101 | N | LYS | C | 234 | −17.942 | 23.106 | −23.626 | 1.00 | 94.85 | C |
| ATOM | 9102 | CA | LYS | C | 234 | −19.193 | 23.041 | −24.375 | 1.00 | 94.85 | C |
| ATOM | 9103 | CB | LYS | C | 234 | −19.934 | 21.739 | −24.073 | 1.00 | 82.61 | C |
| ATOM | 9104 | CG | LYS | C | 234 | −19.263 | 20.545 | −24.740 | 1.00 | 82.61 | C |
| ATOM | 9105 | CD | LYS | C | 234 | −20.010 | 19.240 | −24.507 | 1.00 | 82.61 | C |
| ATOM | 9106 | CE | LYS | C | 234 | −19.318 | 18.097 | −25.241 | 1.00 | 82.61 | C |
| ATOM | 9107 | NZ | LYS | C | 234 | −19.922 | 16.775 | −24.944 | 1.00 | 82.61 | C |
| ATOM | 9108 | C | LYS | C | 234 | −20.069 | 24.257 | −24.097 | 1.00 | 94.85 | C |
| ATOM | 9109 | O | LYS | C | 234 | −20.617 | 24.842 | −25.027 | 1.00 | 94.85 | C |
| ATOM | 9110 | N | ASN | C | 235 | −20.215 | 24.636 | −22.830 | 1.00 | 103.85 | C |
| ATOM | 9111 | CA | ASN | C | 235 | −20.981 | 25.839 | −22.510 | 1.00 | 103.85 | C |
| ATOM | 9112 | CB | ASN | C | 235 | −21.343 | 25.907 | −21.018 | 1.00 | 112.24 | C |
| ATOM | 9113 | CG | ASN | C | 235 | −22.029 | 27.222 | −20.641 | 1.00 | 112.24 | C |
| ATOM | 9114 | OD1 | ASN | C | 235 | −21.584 | 28.286 | −21.071 | 1.00 | 112.24 | C |
| ATOM | 9115 | ND2 | ASN | C | 235 | −23.095 | 27.161 | −19.838 | 1.00 | 112.24 | C |
| ATOM | 9116 | C | ASN | C | 235 | −19.974 | 26.933 | −22.856 | 1.00 | 103.85 | C |
| ATOM | 9117 | O | ASN | C | 235 | −18.970 | 27.102 | −22.166 | 1.00 | 103.85 | C |
| ATOM | 9118 | N | ASP | C | 236 | −20.235 | 27.659 | −23.935 | 1.00 | 159.02 | C |
| ATOM | 9119 | CA | ASP | C | 236 | −19.330 | 28.702 | −24.402 | 1.00 | 159.02 | C |
| ATOM | 9120 | CB | ASP | C | 236 | −19.864 | 29.283 | −25.714 | 1.00 | 133.63 | C |
| ATOM | 9121 | CG | ASP | C | 236 | −18.772 | 29.888 | −26.570 | 1.00 | 133.63 | C |
| ATOM | 9122 | OD1 | ASP | C | 236 | −18.235 | 30.950 | −26.197 | 1.00 | 133.63 | C |
| ATOM | 9123 | OD2 | ASP | C | 236 | −18.447 | 29.292 | −27.617 | 1.00 | 133.63 | C |
| ATOM | 9124 | C | ASP | C | 236 | −19.029 | 29.833 | −23.412 | 1.00 | 159.02 | C |
| ATOM | 9125 | O | ASP | C | 236 | −18.167 | 30.669 | −23.680 | 1.00 | 159.02 | C |
| ATOM | 9126 | N | SER | C | 237 | −19.721 | 29.868 | −22.276 | 1.00 | 108.13 | C |
| ATOM | 9127 | CA | SER | C | 237 | −19.475 | 30.920 | −21.285 | 1.00 | 108.13 | C |
| ATOM | 9128 | CB | SER | C | 237 | −20.777 | 31.328 | −20.589 | 1.00 | 103.74 | C |
| ATOM | 9129 | OG | SER | C | 237 | −21.185 | 30.351 | −19.647 | 1.00 | 103.74 | C |
| ATOM | 9130 | C | SER | C | 237 | −18.453 | 30.480 | −20.231 | 1.00 | 108.13 | C |
| ATOM | 9131 | O | SER | C | 237 | −18.182 | 31.213 | −19.279 | 1.00 | 108.13 | C |
| ATOM | 9132 | N | THR | C | 238 | −17.894 | 29.284 | −20.407 | 1.00 | 100.21 | C |
| ATOM | 9133 | CA | THR | C | 238 | −16.902 | 28.743 | −19.480 | 1.00 | 100.21 | C |
| ATOM | 9134 | CB | THR | C | 238 | −17.380 | 27.427 | −18.840 | 1.00 | 96.52 | C |
| ATOM | 9135 | OG1 | THR | C | 238 | −18.742 | 27.564 | −18.424 | 1.00 | 96.52 | C |
| ATOM | 9136 | CG2 | THR | C | 238 | −16.516 | 27.085 | −17.627 | 1.00 | 96.52 | C |
| ATOM | 9137 | C | THR | C | 238 | −15.604 | 28.450 | −20.222 | 1.00 | 100.21 | C |
| ATOM | 9138 | O | THR | C | 238 | −14.526 | 28.437 | −19.629 | 1.00 | 100.21 | C |
| ATOM | 9139 | N | LYS | C | 239 | −15.728 | 28.208 | −21.524 | 1.00 | 110.91 | C |
| ATOM | 9140 | CA | LYS | C | 239 | −14.591 | 27.904 | −22.385 | 1.00 | 110.91 | C |
| ATOM | 9141 | CB | LYS | C | 239 | −14.818 | 28.509 | −23.778 | 1.00 | 119.38 | C |
| ATOM | 9142 | CG | LYS | C | 239 | −13.727 | 28.184 | −24.792 | 1.00 | 119.38 | C |
| ATOM | 9143 | CD | LYS | C | 239 | −14.132 | 28.589 | −26.201 | 1.00 | 119.38 | C |
| ATOM | 9144 | CE | LYS | C | 239 | −15.346 | 27.801 | −26.667 | 1.00 | 119.38 | C |
| ATOM | 9145 | NZ | LYS | C | 239 | −15.753 | 28.176 | −28.046 | 1.00 | 119.38 | C |
| ATOM | 9146 | C | LYS | C | 239 | −13.276 | 28.405 | −21.794 | 1.00 | 110.91 | C |
| ATOM | 9147 | O | LYS | C | 239 | −13.125 | 29.588 | −21.496 | 1.00 | 110.91 | C |
| ATOM | 9148 | N | THR | C | 240 | −12.331 | 27.488 | −21.620 | 1.00 | 76.06 | C |
| ATOM | 9149 | CA | THR | C | 240 | −11.035 | 27.826 | −21.052 | 1.00 | 76.06 | C |
| ATOM | 9150 | CB | THR | C | 240 | −10.923 | 27.346 | −19.597 | 1.00 | 113.02 | C |
| ATOM | 9151 | OG1 | THR | C | 240 | −12.022 | 27.858 | −18.838 | 1.00 | 113.02 | C |
| ATOM | 9152 | CG2 | THR | C | 240 | −9.621 | 27.824 | −18.981 | 1.00 | 113.02 | C |
| ATOM | 9153 | C | THR | C | 240 | −9.884 | 27.204 | −21.822 | 1.00 | 76.06 | C |
| ATOM | 9154 | O | THR | C | 240 | −10.033 | 26.169 | −22.476 | 1.00 | 76.06 | C |
| ATOM | 9155 | N | THR | C | 241 | −8.727 | 27.845 | −21.717 | 1.00 | 78.68 | C |
| ATOM | 9156 | CA | THR | C | 241 | −7.515 | 27.390 | −22.377 | 1.00 | 78.68 | C |
| ATOM | 9157 | CB | THR | C | 241 | −6.410 | 28.447 | −22.252 | 1.00 | 109.14 | C |
| ATOM | 9158 | OG1 | THR | C | 241 | −6.940 | 29.732 | −22.599 | 1.00 | 68.84 | C |
| ATOM | 9159 | CG2 | THR | C | 241 | −5.226 | 28.097 | −23.156 | 1.00 | 68.84 | C |
| ATOM | 9160 | C | THR | C | 241 | −6.991 | 26.106 | −21.737 | 1.00 | 78.68 | C |
| ATOM | 9161 | O | THR | C | 241 | −6.610 | 25.160 | −22.429 | 1.00 | 78.68 | C |
| ATOM | 9162 | N | SER | C | 242 | −6.978 | 26.089 | −20.406 | 1.00 | 85.12 | C |
| ATOM | 9163 | CA | SER | C | 242 | −6.458 | 24.947 | −19.669 | 1.00 | 85.12 | C |
| ATOM | 9164 | CB | SER | C | 242 | −4.972 | 25.160 | −19.396 | 1.00 | 131.02 | C |
| ATOM | 9165 | OG | SER | C | 242 | −4.781 | 26.312 | −18.589 | 1.00 | 103.54 | C |
| ATOM | 9166 | C | SER | C | 242 | −7.154 | 24.664 | −18.343 | 1.00 | 85.12 | C |
| ATOM | 9167 | O | SER | C | 242 | −7.961 | 25.456 | −17.849 | 1.00 | 85.12 | C |
| ATOM | 9168 | N | LEU | C | 243 | −6.798 | 23.519 | −17.770 | 1.00 | 67.36 | C |
| ATOM | 9169 | CA | LEU | C | 243 | −7.344 | 23.069 | −16.503 | 1.00 | 67.36 | C |
| ATOM | 9170 | CB | LEU | C | 243 | −8.361 | 21.961 | −16.758 | 1.00 | 119.22 | C |
| ATOM | 9171 | CG | LEU | C | 243 | −9.236 | 21.563 | −15.577 | 1.00 | 95.35 | C |
| ATOM | 9172 | CD1 | LEU | C | 243 | −10.005 | 22.779 | −15.090 | 1.00 | 95.35 | C |
| ATOM | 9173 | CD2 | LEU | C | 243 | −10.189 | 20.454 | −16.003 | 1.00 | 95.35 | C |
| ATOM | 9174 | C | LEU | C | 243 | −6.188 | 22.537 | −15.648 | 1.00 | 67.36 | C |
| ATOM | 9175 | O | LEU | C | 243 | −5.280 | 21.868 | −16.157 | 1.00 | 67.36 | C |
| ATOM | 9176 | N | SER | C | 244 | −6.213 | 22.841 | −14.353 | 1.00 | 62.79 | C |
| ATOM | 9177 | CA | SER | C | 244 | −5.158 | 22.384 | −13.457 | 1.00 | 62.79 | C |
| ATOM | 9178 | CB | SER | C | 244 | −4.243 | 23.545 | −13.073 | 1.00 | 74.79 | C |
| ATOM | 9179 | OG | SER | C | 244 | −3.489 | 23.996 | −14.173 | 1.00 | 76.94 | C |
| ATOM | 9180 | C | SER | C | 244 | −5.665 | 21.746 | −12.172 | 1.00 | 62.79 | C |
| ATOM | 9181 | O | SER | C | 244 | −6.659 | 22.190 | −11.571 | 1.00 | 62.79 | C |
| ATOM | 9182 | N | LEU | C | 245 | −4.965 | 20.699 | −11.752 | 1.00 | 60.79 | C |
| ATOM | 9183 | CA | LEU | C | 245 | −5.293 | 20.022 | −10.509 | 1.00 | 60.79 | C |
| ATOM | 9184 | CB | LEU | C | 245 | −5.389 | 18.509 | −10.708 | 1.00 | 76.51 | C |
| ATOM | 9185 | CG | LEU | C | 245 | −5.643 | 17.743 | −9.404 | 1.00 | 60.50 | C |
| ATOM | 9186 | CD1 | LEU | C | 245 | −6.916 | 18.257 | −8.746 | 1.00 | 60.50 | C |
| ATOM | 9187 | CD2 | LEU | C | 245 | −5.741 | 16.256 | −9.693 | 1.00 | 60.50 | C |
| ATOM | 9188 | C | LEU | C | 245 | −4.164 | 20.338 | −9.539 | 1.00 | 60.79 | C |
| ATOM | 9189 | O | LEU | C | 245 | −3.052 | 19.827 | −9.683 | 1.00 | 60.79 | C |
| ATOM | 9190 | N | ILE | C | 246 | −4.447 | 21.197 | −8.565 | 1.00 | 54.76 | C |
| ATOM | 9191 | CA | ILE | C | 246 | −3.446 | 21.584 | −7.577 | 1.00 | 54.76 | C |
| ATOM | 9192 | CB | ILE | C | 246 | −3.477 | 23.112 | −7.322 | 1.00 | 68.26 | C |
| ATOM | 9193 | CG2 | ILE | C | 246 | −2.348 | 23.517 | −6.381 | 1.00 | 51.30 | C |
| ATOM | 9194 | CG1 | ILE | C | 246 | −3.340 | 23.854 | −8.649 | 1.00 | 51.30 | C |
| ATOM | 9195 | CD1 | ILE | C | 246 | −2.090 | 23.475 | −9.428 | 1.00 | 51.30 | C |
| ATOM | 9196 | C | ILE | C | 246 | −3.684 | 20.861 | −6.254 | 1.00 | 54.76 | C |
| ATOM | 9197 | O | ILE | C | 246 | −4.731 | 21.030 | −5.617 | 1.00 | 54.76 | C |
| ATOM | 9198 | N | THR | C | 247 | −2.714 | 20.052 | −5.846 | 1.00 | 49.03 | C |
| ATOM | 9199 | CA | THR | C | 247 | −2.848 | 19.324 | −4.603 | 1.00 | 49.03 | C |
| ATOM | 9200 | CB | THR | C | 247 | −3.428 | 17.930 | −4.859 | 1.00 | 65.84 | C |
| ATOM | 9201 | OG1 | THR | C | 247 | −3.806 | 17.340 | −3.615 | 1.00 | 63.08 | C |
| ATOM | 9202 | CG2 | THR | C | 247 | −2.401 | 17.041 | −5.545 | 1.00 | 63.08 | C |
| ATOM | 9203 | C | THR | C | 247 | −1.479 | 19.214 | −3.928 | 1.00 | 49.03 | C |
| ATOM | 9204 | O | THR | C | 247 | −0.450 | 19.160 | −4.604 | 1.00 | 49.03 | C |
| ATOM | 9205 | N | GLY | C | 248 | −1.479 | 19.198 | −2.597 | 1.00 | 51.61 | C |
| ATOM | 9206 | CA | GLY | C | 248 | −0.237 | 19.113 | −1.850 | 1.00 | 51.61 | C |
| ATOM | 9207 | C | GLY | C | 248 | −0.438 | 19.459 | −0.381 | 1.00 | 51.61 | C |
| ATOM | 9208 | O | GLY | C | 248 | −1.573 | 19.596 | 0.091 | 1.00 | 51.61 | C |
| ATOM | 9209 | N | CYS | C | 249 | 0.657 | 19.602 | 0.357 | 1.00 | 57.84 | C |
| ATOM | 9210 | CA | CYS | C | 249 | 0.555 | 19.921 | 1.774 | 1.00 | 57.84 | C |
| ATOM | 9211 | CB | CYS | C | 249 | 0.539 | 18.632 | 2.607 | 1.00 | 40.23 | C |
| ATOM | 9212 | SG | CYS | C | 249 | 2.131 | 17.774 | 2.665 | 1.00 | 59.00 | C |
| ATOM | 9213 | C | CYS | C | 249 | 1.709 | 20.806 | 2.225 | 1.00 | 57.84 | C |
| ATOM | 9214 | O | CYS | C | 249 | 2.605 | 21.140 | 1.440 | 1.00 | 57.84 | C |
| ATOM | 9215 | N | TYR | C | 250 | 1.695 | 21.166 | 3.503 | 1.00 | 51.74 | C |
| ATOM | 9216 | CA | TYR | C | 250 | 2.731 | 22.030 | 4.042 | 1.00 | 51.74 | C |
| ATOM | 9217 | CB | TYR | C | 250 | 2.391 | 23.470 | 3.724 | 1.00 | 35.90 | C |
| ATOM | 9218 | CG | TYR | C | 250 | 1.095 | 23.903 | 4.371 | 1.00 | 26.25 | C |
| ATOM | 9219 | CD1 | TYR | C | 250 | 1.082 | 24.471 | 5.640 | 1.00 | 26.25 | C |
| ATOM | 9220 | CE1 | TYR | C | 250 | −0.117 | 24.870 | 6.241 | 1.00 | 26.25 | C |
| ATOM | 9221 | CD2 | TYR | C | 250 | −0.126 | 23.735 | 3.712 | 1.00 | 26.25 | C |
| ATOM | 9222 | CE2 | TYR | C | 250 | −1.327 | 24.127 | 4.302 | 1.00 | 26.25 | C |
| ATOM | 9223 | CZ | TYR | C | 250 | −1.312 | 24.696 | 5.564 | 1.00 | 26.25 | C |
| ATOM | 9224 | OH | TYR | C | 250 | −2.483 | 25.114 | 6.158 | 1.00 | 26.25 | C |
| ATOM | 9225 | C | TYR | C | 250 | 2.896 | 21.928 | 5.542 | 1.00 | 51.74 | C |
| ATOM | 9226 | O | TYR | C | 250 | 1.960 | 21.591 | 6.265 | 1.00 | 51.74 | C |
| ATOM | 9227 | N | GLU | C | 251 | 4.102 | 22.248 | 5.990 | 1.00 | 69.66 | C |
| ATOM | 9228 | CA | GLU | C | 251 | 4.449 | 22.272 | 7.396 | 1.00 | 69.66 | C |
| ATOM | 9229 | CB | GLU | C | 251 | 5.322 | 21.075 | 7.772 | 1.00 | 136.16 | C |
| ATOM | 9230 | CG | GLU | C | 251 | 4.627 | 20.111 | 8.719 | 1.00 | 89.78 | C |
| ATOM | 9231 | CD | GLU | C | 251 | 5.542 | 19.007 | 9.232 | 1.00 | 89.78 | C |
| ATOM | 9232 | OE1 | GLU | C | 251 | 6.601 | 19.328 | 9.820 | 1.00 | 89.78 | C |
| ATOM | 9233 | OE2 | GLU | C | 251 | 5.196 | 17.815 | 9.055 | 1.00 | 89.78 | C |
| ATOM | 9234 | C | GLU | C | 251 | 5.230 | 23.564 | 7.521 | 1.00 | 69.66 | C |
| ATOM | 9235 | O | GLU | C | 251 | 6.458 | 23.567 | 7.478 | 1.00 | 69.66 | C |
| ATOM | 9236 | N | THR | C | 252 | 4.495 | 24.666 | 7.629 | 1.00 | 55.04 | C |
| ATOM | 9237 | CA | THR | C | 252 | 5.070 | 26.004 | 7.755 | 1.00 | 55.04 | C |
| ATOM | 9238 | CB | THR | C | 252 | 4.193 | 27.029 | 7.007 | 1.00 | 65.80 | C |
| ATOM | 9239 | OG1 | THR | C | 252 | 3.984 | 26.588 | 5.662 | 1.00 | 65.80 | C |
| ATOM | 9240 | CG2 | THR | C | 252 | 4.860 | 28.389 | 6.984 | 1.00 | 65.80 | C |
| ATOM | 9241 | C | THR | C | 252 | 5.125 | 26.383 | 9.239 | 1.00 | 55.04 | C |
| ATOM | 9242 | O | THR | C | 252 | 4.144 | 26.876 | 9.799 | 1.00 | 55.04 | C |
| ATOM | 9243 | N | GLY | C | 253 | 6.269 | 26.165 | 9.876 | 1.00 | 58.47 | C |
| ATOM | 9244 | CA | GLY | C | 253 | 6.356 | 26.466 | 11.292 | 1.00 | 58.47 | C |
| ATOM | 9245 | C | GLY | C | 253 | 5.389 | 25.569 | 12.052 | 1.00 | 58.47 | C |
| ATOM | 9246 | O | GLY | C | 253 | 5.393 | 24.350 | 11.869 | 1.00 | 58.47 | C |
| ATOM | 9247 | N | LEU | C | 254 | 4.556 | 26.159 | 12.901 | 1.00 | 61.58 | C |
| ATOM | 9248 | CA | LEU | C | 254 | 3.584 | 25.387 | 13.661 | 1.00 | 61.58 | C |
| ATOM | 9249 | CB | LEU | C | 254 | 3.048 | 26.184 | 14.845 | 1.00 | 36.25 | C |
| ATOM | 9250 | CG | LEU | C | 254 | 3.978 | 26.449 | 16.014 | 1.00 | 36.25 | C |
| ATOM | 9251 | CD1 | LEU | C | 254 | 3.161 | 27.060 | 17.123 | 1.00 | 36.25 | C |
| ATOM | 9252 | CD2 | LEU | C | 254 | 4.636 | 25.175 | 16.467 | 1.00 | 36.25 | C |
| ATOM | 9253 | C | LEU | C | 254 | 2.398 | 24.989 | 12.802 | 1.00 | 61.58 | C |
| ATOM | 9254 | O | LEU | C | 254 | 1.762 | 23.970 | 13.066 | 1.00 | 61.58 | C |
| ATOM | 9255 | N | LEU | C | 255 | 2.089 | 25.801 | 11.793 | 1.00 | 48.24 | C |
| ATOM | 9256 | CA | LEU | C | 255 | 0.962 | 25.533 | 10.909 | 1.00 | 48.24 | C |
| ATOM | 9257 | CB | LEU | C | 255 | 0.648 | 26.761 | 10.065 | 1.00 | 42.35 | C |
| ATOM | 9258 | CG | LEU | C | 255 | 0.459 | 28.072 | 10.830 | 1.00 | 42.35 | C |
| ATOM | 9259 | CD1 | LEU | C | 255 | 0.070 | 29.166 | 9.856 | 1.00 | 42.35 | C |
| ATOM | 9260 | CD2 | LEU | C | 255 | −0.605 | 27.917 | 11.900 | 1.00 | 42.35 | C |
| ATOM | 9261 | C | LEU | C | 255 | 1.211 | 24.343 | 9.987 | 1.00 | 48.24 | C |
| ATOM | 9262 | O | LEU | C | 255 | 2.353 | 24.051 | 9.613 | 1.00 | 48.24 | C |
| ATOM | 9263 | N | PHE | C | 256 | 0.132 | 23.651 | 9.630 | 1.00 | 49.21 | C |
| ATOM | 9264 | CA | PHE | C | 256 | 0.225 | 22.493 | 8.755 | 1.00 | 49.21 | C |
| ATOM | 9265 | CB | PHE | C | 256 | 0.794 | 21.290 | 9.518 | 1.00 | 47.20 | C |
| ATOM | 9266 | CG | PHE | C | 256 | −0.102 | 20.769 | 10.613 | 1.00 | 44.21 | C |
| ATOM | 9267 | CD1 | PHE | C | 256 | −0.944 | 19.693 | 10.387 | 1.00 | 44.21 | C |
| ATOM | 9268 | CD2 | PHE | C | 256 | −0.095 | 21.350 | 11.884 | 1.00 | 44.21 | C |
| ATOM | 9269 | CE1 | PHE | C | 256 | −1.757 | 19.206 | 11.403 | 1.00 | 44.21 | C |
| ATOM | 9270 | CE2 | PHE | C | 256 | −0.915 | 20.860 | 12.909 | 1.00 | 44.21 | C |
| ATOM | 9271 | CZ | PHE | C | 256 | −1.741 | 19.793 | 12.665 | 1.00 | 44.21 | C |
| ATOM | 9272 | C | PHE | C | 256 | −1.144 | 22.161 | 8.181 | 1.00 | 49.21 | C |
| ATOM | 9273 | O | PHE | C | 256 | −2.163 | 22.319 | 8.850 | 1.00 | 49.21 | C |
| ATOM | 9274 | N | GLY | C | 257 | −1.164 | 21.714 | 6.929 | 1.00 | 41.97 | C |
| ATOM | 9275 | CA | GLY | C | 257 | −2.417 | 21.370 | 6.286 | 1.00 | 41.97 | C |
| ATOM | 9276 | C | GLY | C | 257 | −2.187 | 20.770 | 4.911 | 1.00 | 41.97 | C |
| ATOM | 9277 | O | GLY | C | 257 | −1.037 | 20.590 | 4.483 | 1.00 | 41.97 | C |
| ATOM | 9278 | N | SER | C | 258 | −3.276 | 20.450 | 4.218 | 1.00 | 32.22 | C |
| ATOM | 9279 | CA | SER | C | 258 | −3.185 | 19.883 | 2.875 | 1.00 | 32.22 | C |
| ATOM | 9280 | CB | SER | C | 258 | −3.357 | 18.377 | 2.921 | 1.00 | 34.80 | C |
| ATOM | 9281 | OG | SER | C | 258 | −4.689 | 18.047 | 3.277 | 1.00 | 44.92 | C |
| ATOM | 9282 | C | SER | C | 258 | −4.293 | 20.494 | 2.029 | 1.00 | 32.22 | C |
| ATOM | 9283 | O | SER | C | 258 | −5.267 | 21.029 | 2.562 | 1.00 | 32.22 | C |
| ATOM | 9284 | N | TYR | C | 259 | −4.154 | 20.409 | 0.715 | 1.00 | 48.87 | C |
| ATOM | 9285 | CA | TYR | C | 259 | −5.150 | 20.994 | −0.175 | 1.00 | 48.87 | C |
| ATOM | 9286 | CB | TYR | C | 259 | −4.775 | 22.450 | −0.499 | 1.00 | 49.67 | C |
| ATOM | 9287 | CG | TYR | C | 259 | −3.364 | 22.584 | −1.018 | 1.00 | 45.42 | C |
| ATOM | 9288 | CD1 | TYR | C | 259 | −3.056 | 22.303 | −2.353 | 1.00 | 45.42 | C |
| ATOM | 9289 | CE1 | TYR | C | 259 | −1.744 | 22.304 | −2.798 | 1.00 | 45.42 | C |
| ATOM | 9290 | CD2 | TYR | C | 259 | −2.317 | 22.878 | −0.152 | 1.00 | 45.42 | C |
| ATOM | 9291 | CE2 | TYR | C | 259 | −1.006 | 22.878 | −0.592 | 1.00 | 45.42 | C |
| ATOM | 9292 | CZ | TYR | C | 259 | −0.726 | 22.587 | −1.909 | 1.00 | 45.42 | C |
| ATOM | 9293 | OH | TYR | C | 259 | 0.585 | 22.554 | −2.313 | 1.00 | 45.42 | C |
| ATOM | 9294 | C | TYR | C | 259 | −5.271 | 20.209 | −1.462 | 1.00 | 48.87 | C |
| ATOM | 9295 | O | TYR | C | 259 | −4.397 | 19.410 | −1.818 | 1.00 | 48.87 | C |
| ATOM | 9296 | N | GLY | C | 260 | −6.370 | 20.455 | −2.159 | 1.00 | 45.95 | C |
| ATOM | 9297 | CA | GLY | C | 260 | −6.626 | 19.791 | −3.419 | 1.00 | 45.95 | C |
| ATOM | 9298 | C | GLY | C | 260 | −7.749 | 20.542 | −4.099 | 1.00 | 45.95 | C |
| ATOM | 9299 | O | GLY | C | 260 | −8.910 | 20.451 | −3.698 | 1.00 | 45.95 | C |
| ATOM | 9300 | N | TYR | C | 261 | −7.406 | 21.316 | −5.116 | 1.00 | 49.08 | C |
| ATOM | 9301 | CA | TYR | C | 261 | −8.425 | 22.072 | −5.821 | 1.00 | 49.08 | C |
| ATOM | 9302 | CB | TYR | C | 261 | −8.522 | 23.490 | −5.254 | 1.00 | 77.59 | C |
| ATOM | 9303 | CG | TYR | C | 261 | −7.256 | 24.300 | −5.379 | 1.00 | 61.38 | C |
| ATOM | 9304 | CD1 | TYR | C | 261 | −6.982 | 25.035 | −6.526 | 1.00 | 61.38 | C |
| ATOM | 9305 | CE1 | TYR | C | 261 | −5.824 | 25.799 | −6.631 | 1.00 | 61.38 | C |
| ATOM | 9306 | CD2 | TYR | C | 261 | −6.337 | 24.345 | −4.340 | 1.00 | 61.38 | C |
| ATOM | 9307 | CE2 | TYR | C | 261 | −5.176 | 25.105 | −4.431 | 1.00 | 61.38 | C |
| ATOM | 9308 | CZ | TYR | C | 261 | −4.922 | 25.833 | −5.575 | 1.00 | 61.38 | C |
| ATOM | 9309 | OH | TYR | C | 261 | −3.778 | 26.604 | −5.640 | 1.00 | 61.38 | C |
| ATOM | 9310 | C | TYR | C | 261 | −8.161 | 22.106 | −7.321 | 1.00 | 49.08 | C |
| ATOM | 9311 | O | TYR | C | 261 | −7.061 | 21.782 | −7.794 | 1.00 | 49.08 | C |
| ATOM | 9312 | N | VAL | C | 262 | −9.193 | 22.480 | −8.062 | 1.00 | 60.83 | C |
| ATOM | 9313 | CA | VAL | C | 262 | −9.108 | 22.556 | −9.506 | 1.00 | 60.83 | C |
| ATOM | 9314 | CB | VAL | C | 262 | −10.222 | 21.727 | −10.160 | 1.00 | 69.54 | C |
| ATOM | 9315 | CG1 | VAL | C | 262 | −10.291 | 22.024 | −11.638 | 1.00 | 61.03 | C |
| ATOM | 9316 | CG2 | VAL | C | 262 | −9.968 | 20.254 | −9.941 | 1.00 | 61.03 | C |
| ATOM | 9317 | C | VAL | C | 262 | −9.277 | 23.998 | −9.943 | 1.00 | 60.83 | C |
| ATOM | 9318 | O | VAL | C | 262 | −10.278 | 24.635 | −9.610 | 1.00 | 60.83 | C |
| ATOM | 9319 | N | GLU | C | 263 | −8.298 | 24.524 | −10.673 | 1.00 | 85.01 | C |
| ATOM | 9320 | CA | GLU | C | 263 | −8.402 | 25.890 | −11.160 | 1.00 | 85.01 | C |
| ATOM | 9321 | CB | GLU | C | 263 | −7.290 | 26.785 | −10.601 | 1.00 | 104.41 | C |
| ATOM | 9322 | CG | GLU | C | 263 | −5.887 | 26.280 | −10.817 | 1.00 | 91.01 | C |
| ATOM | 9323 | CD | GLU | C | 263 | −4.842 | 27.234 | −10.257 | 1.00 | 91.01 | C |
| ATOM | 9324 | OE1 | GLU | C | 263 | −5.060 | 27.785 | −9.149 | 1.00 | 91.01 | C |
| ATOM | 9325 | OE2 | GLU | C | 263 | −3.796 | 27.421 | −10.920 | 1.00 | 91.01 | C |
| ATOM | 9326 | C | GLU | C | 263 | −8.384 | 25.905 | −12.677 | 1.00 | 85.01 | C |
| ATOM | 9327 | O | GLU | C | 263 | −7.942 | 24.955 | −13.330 | 1.00 | 85.01 | C |
| ATOM | 9328 | N | THR | C | 264 | −8.886 | 27.003 | −13.220 | 1.00 | 101.12 | C |
| ATOM | 9329 | CA | THR | C | 264 | −8.991 | 27.211 | −14.650 | 1.00 | 101.12 | C |
| ATOM | 9330 | CB | THR | C | 264 | −10.472 | 27.464 | −14.993 | 1.00 | 93.50 | C |
| ATOM | 9331 | OG1 | THR | C | 264 | −11.051 | 26.252 | −15.483 | 1.00 | 72.43 | C |
| ATOM | 9332 | CG2 | THR | C | 264 | −10.636 | 28.567 | −15.999 | 1.00 | 72.43 | C |
| ATOM | 9333 | C | THR | C | 264 | −8.130 | 28.399 | −15.047 | 1.00 | 101.12 | C |
| ATOM | 9334 | O | THR | C | 264 | −7.676 | 29.147 | −14.189 | 1.00 | 101.12 | C |
| ATOM | 9335 | N | ALA | C | 265 | −7.886 | 28.569 | −16.340 | 1.00 | 76.82 | C |
| ATOM | 9336 | CA | ALA | C | 265 | −7.088 | 29.703 | −16.790 | 1.00 | 76.82 | C |
| ATOM | 9337 | CB | ALA | C | 265 | −6.917 | 29.663 | −18.310 | 1.00 | 58.52 | C |
| ATOM | 9338 | C | ALA | C | 265 | −7.806 | 30.987 | −16.360 | 1.00 | 76.82 | C |
| ATOM | 9339 | O | ALA | C | 265 | −7.193 | 32.043 | −16.229 | 1.00 | 76.82 | C |
| ATOM | 9340 | N | ASN | C | 266 | −9.108 | 30.877 | −16.119 | 1.00 | 85.95 | C |
| ATOM | 9341 | CA | ASN | C | 266 | −9.922 | 32.015 | −15.706 | 1.00 | 85.95 | C |
| ATOM | 9342 | CB | ASN | C | 266 | −11.373 | 31.819 | −16.150 | 1.00 | 165.17 | C |
| ATOM | 9343 | CG | ASN | C | 266 | −11.495 | 31.560 | −17.637 | 1.00 | 107.02 | C |
| ATOM | 9344 | OD1 | ASN | C | 266 | −10.973 | 32.316 | −18.454 | 1.00 | 107.02 | C |
| ATOM | 9345 | ND2 | ASN | C | 266 | −12.191 | 30.487 | −17.996 | 1.00 | 107.02 | C |
| ATOM | 9346 | C | ASN | C | 266 | −9.891 | 32.227 | −14.199 | 1.00 | 85.95 | C |
| ATOM | 9347 | O | ASN | C | 266 | −10.330 | 33.266 | −13.707 | 1.00 | 85.95 | C |
| ATOM | 9348 | N | GLY | C | 267 | −9.386 | 31.238 | −13.467 | 1.00 | 111.63 | C |
| ATOM | 9349 | CA | GLY | C | 267 | −9.310 | 31.361 | −12.021 | 1.00 | 111.63 | C |
| ATOM | 9350 | C | GLY | C | 267 | −9.798 | 30.144 | −11.260 | 1.00 | 111.63 | C |
| ATOM | 9351 | O | GLY | C | 267 | −10.327 | 29.205 | −11.858 | 1.00 | 111.63 | C |
| ATOM | 9352 | N | PRO | C | 268 | −9.643 | 30.140 | −9.926 | 1.00 | 81.13 | C |
| ATOM | 9353 | CD | PRO | C | 268 | −9.287 | 31.293 | −9.087 | 1.00 | 81.08 | C |
| ATOM | 9354 | CA | PRO | C | 268 | −10.075 | 29.018 | −9.091 | 1.00 | 81.13 | C |
| ATOM | 9355 | CB | PRO | C | 268 | −9.945 | 29.571 | −7.671 | 1.00 | 96.97 | C |
| ATOM | 9356 | CG | PRO | C | 268 | −10.114 | 31.036 | −7.857 | 1.00 | 81.08 | C |
| ATOM | 9357 | C | PRO | C | 268 | −11.488 | 28.550 | −9.414 | 1.00 | 81.13 | C |
| ATOM | 9358 | O | PRO | C | 268 | −12.398 | 29.357 | −9.589 | 1.00 | 81.13 | C |
| ATOM | 9359 | N | LEU | C | 269 | −11.657 | 27.234 | −9.477 | 1.00 | 64.68 | C |
| ATOM | 9360 | CA | LEU | C | 269 | −12.941 | 26.636 | −9.796 | 1.00 | 64.68 | C |
| ATOM | 9361 | CB | LEU | C | 269 | −12.755 | 25.662 | −10.959 | 1.00 | 97.46 | C |
| ATOM | 9362 | CG | LEU | C | 269 | −14.013 | 25.228 | −11.696 | 1.00 | 62.80 | C |
| ATOM | 9363 | CD1 | LEU | C | 269 | −14.790 | 26.459 | −12.142 | 1.00 | 62.80 | C |
| ATOM | 9364 | CD2 | LEU | C | 269 | −13.621 | 24.357 | −12.884 | 1.00 | 62.80 | C |
| ATOM | 9365 | C | LEU | C | 269 | −13.582 | 25.925 | −8.603 | 1.00 | 64.68 | C |
| ATOM | 9366 | O | LEU | C | 269 | −14.549 | 26.426 | −8.024 | 1.00 | 64.68 | C |
| ATOM | 9367 | N | ALA | C | 270 | −13.039 | 24.767 | −8.231 | 1.00 | 63.31 | C |
| ATOM | 9368 | CA | ALA | C | 270 | −13.577 | 23.990 | −7.113 | 1.00 | 63.31 | C |
| ATOM | 9369 | CB | ALA | C | 270 | −14.492 | 22.885 | −7.637 | 1.00 | 83.13 | C |
| ATOM | 9370 | C | ALA | C | 270 | −12.511 | 23.382 | −6.205 | 1.00 | 63.31 | C |
| ATOM | 9371 | O | ALA | C | 270 | −11.445 | 22.948 | −6.655 | 1.00 | 63.31 | C |
| ATOM | 9372 | N | ARG | C | 271 | −12.841 | 23.337 | −4.921 | 1.00 | 63.18 | C |
| ATOM | 9373 | CA | ARG | C | 271 | −11.964 | 22.818 | −3.885 | 1.00 | 63.18 | C |
| ATOM | 9374 | CB | ARG | C | 271 | −11.881 | 23.871 | −2.782 | 1.00 | 61.94 | C |
| ATOM | 9375 | CG | ARG | C | 271 | −11.246 | 23.434 | −1.500 | 1.00 | 55.06 | C |
| ATOM | 9376 | CD | ARG | C | 271 | −11.324 | 24.547 | −0.454 | 1.00 | 55.06 | C |
| ATOM | 9377 | NE | ARG | C | 271 | −10.471 | 25.688 | −0.784 | 1.00 | 55.06 | C |
| ATOM | 9378 | CZ | ARG | C | 271 | −10.914 | 26.861 | −1.234 | 1.00 | 55.06 | C |
| ATOM | 9379 | NH1 | ARG | C | 271 | −12.210 | 27.064 | −1.417 | 1.00 | 55.06 | C |
| ATOM | 9380 | NH2 | ARG | C | 271 | −10.060 | 27.842 | −1.481 | 1.00 | 55.06 | C |
| ATOM | 9381 | C | ARG | C | 271 | −12.525 | 21.495 | −3.345 | 1.00 | 63.18 | C |
| ATOM | 9382 | O | ARG | C | 271 | −13.727 | 21.382 | −3.086 | 1.00 | 63.18 | C |
| ATOM | 9383 | N | TYR | C | 272 | −11.663 | 20.492 | −3.185 | 1.00 | 50.25 | C |
| ATOM | 9384 | CA | TYR | C | 272 | −12.096 | 19.190 | −2.680 | 1.00 | 50.25 | C |
| ATOM | 9385 | CB | TYR | C | 272 | −11.228 | 18.077 | −3.260 | 1.00 | 68.10 | C |
| ATOM | 9386 | CG | TYR | C | 272 | −11.677 | 16.697 | −2.831 | 1.00 | 65.57 | C |
| ATOM | 9387 | CD1 | TYR | C | 272 | −12.746 | 16.060 | −3.462 | 1.00 | 65.57 | C |
| ATOM | 9388 | CE1 | TYR | C | 272 | −13.183 | 14.799 | −3.049 | 1.00 | 65.57 | C |
| ATOM | 9389 | CD2 | TYR | C | 272 | −11.055 | 16.037 | −1.773 | 1.00 | 65.57 | C |
| ATOM | 9390 | CE2 | TYR | C | 272 | −11.486 | 14.777 | −1.354 | 1.00 | 65.57 | C |
| ATOM | 9391 | CZ | TYR | C | 272 | −12.548 | 14.168 | −1.999 | 1.00 | 65.57 | C |
| ATOM | 9392 | OH | TYR | C | 272 | −12.971 | 12.926 | −1.606 | 1.00 | 65.57 | C |
| ATOM | 9393 | C | TYR | C | 272 | −12.040 | 19.102 | −1.155 | 1.00 | 50.25 | C |
| ATOM | 9394 | O | TYR | C | 272 | −10.979 | 19.281 | −0.553 | 1.00 | 50.25 | C |
| ATOM | 9395 | N | HIS | C | 273 | −13.173 | 18.817 | −0.524 | 1.00 | 69.22 | C |
| ATOM | 9396 | CA | HIS | C | 273 | −13.196 | 18.714 | 0.929 | 1.00 | 69.22 | C |
| ATOM | 9397 | CB | HIS | C | 273 | −14.415 | 19.430 | 1.498 | 1.00 | 114.55 | C |
| ATOM | 9398 | CG | HIS | C | 273 | −14.282 | 20.919 | 1.490 | 1.00 | 95.28 | C |
| ATOM | 9399 | CD2 | HIS | C | 273 | −15.021 | 21.879 | 0.889 | 1.00 | 95.28 | C |
| ATOM | 9400 | ND1 | HIS | C | 273 | −13.261 | 21.575 | 2.144 | 1.00 | 95.28 | C |
| ATOM | 9401 | CE1 | HIS | C | 273 | −13.376 | 22.876 | 1.945 | 1.00 | 95.28 | C |
| ATOM | 9402 | NE2 | HIS | C | 273 | −14.436 | 23.087 | 1.186 | 1.00 | 95.28 | C |
| ATOM | 9403 | C | HIS | C | 273 | −13.159 | 17.281 | 1.416 | 1.00 | 69.22 | C |
| ATOM | 9404 | O | HIS | C | 273 | −14.027 | 16.475 | 1.096 | 1.00 | 69.22 | C |
| ATOM | 9405 | N | THR | C | 274 | −12.122 | 16.981 | 2.185 | 1.00 | 62.23 | C |
| ATOM | 9406 | CA | THR | C | 274 | −11.902 | 15.663 | 2.757 | 1.00 | 62.23 | C |
| ATOM | 9407 | CB | THR | C | 274 | −10.615 | 15.681 | 3.613 | 1.00 | 75.75 | C |
| ATOM | 9408 | OG1 | THR | C | 274 | −9.520 | 15.183 | 2.828 | 1.00 | 68.28 | C |
| ATOM | 9409 | CG2 | THR | C | 274 | −10.786 | 14.866 | 4.889 | 1.00 | 68.28 | C |
| ATOM | 9410 | C | THR | C | 274 | −13.083 | 15.190 | 3.601 | 1.00 | 62.23 | C |
| ATOM | 9411 | O | THR | C | 274 | −13.411 | 13.999 | 3.619 | 1.00 | 62.23 | C |
| ATOM | 9412 | N | GLY | C | 275 | −13.713 | 16.126 | 4.301 | 1.00 | 60.19 | C |
| ATOM | 9413 | CA | GLY | C | 275 | −14.851 | 15.783 | 5.131 | 1.00 | 60.19 | C |
| ATOM | 9414 | C | GLY | C | 275 | −16.066 | 15.346 | 4.334 | 1.00 | 60.19 | C |
| ATOM | 9415 | O | GLY | C | 275 | −16.492 | 14.200 | 4.428 | 1.00 | 60.19 | C |
| ATOM | 9416 | N | ASP | C | 276 | −16.613 | 16.253 | 3.535 | 1.00 | 66.63 | C |
| ATOM | 9417 | CA | ASP | C | 276 | −17.795 | 15.980 | 2.721 | 1.00 | 66.63 | C |
| ATOM | 9418 | CB | ASP | C | 276 | −18.323 | 17.292 | 2.134 | 1.00 | 174.71 | C |
| ATOM | 9419 | CG | ASP | C | 276 | −18.461 | 18.381 | 3.178 | 1.00 | 153.71 | C |
| ATOM | 9420 | OD1 | ASP | C | 276 | −19.338 | 18.261 | 4.066 | 1.00 | 153.71 | C |
| ATOM | 9421 | OD2 | ASP | C | 276 | −17.680 | 19.356 | 3.114 | 1.00 | 153.71 | C |
| ATOM | 9422 | C | ASP | C | 276 | −17.562 | 14.981 | 1.585 | 1.00 | 66.63 | C |
| ATOM | 9423 | O | ASP | C | 276 | −18.518 | 14.419 | 1.046 | 1.00 | 66.63 | C |
| ATOM | 9424 | N | LYS | C | 277 | −16.300 | 14.761 | 1.223 | 1.00 | 70.79 | C |
| ATOM | 9425 | CA | LYS | C | 277 | −15.949 | 13.849 | 0.139 | 1.00 | 70.79 | C |
| ATOM | 9426 | CB | LYS | C | 277 | −16.381 | 12.429 | 0.491 | 1.00 | 107.51 | C |
| ATOM | 9427 | CG | LYS | C | 277 | −15.596 | 11.831 | 1.647 | 1.00 | 92.72 | C |
| ATOM | 9428 | CD | LYS | C | 277 | −16.270 | 10.572 | 2.176 | 1.00 | 92.72 | C |
| ATOM | 9429 | CE | LYS | C | 277 | −15.466 | 9.902 | 3.286 | 1.00 | 92.72 | C |
| ATOM | 9430 | NZ | LYS | C | 277 | −14.257 | 9.195 | 2.768 | 1.00 | 92.72 | C |
| ATOM | 9431 | C | LYS | C | 277 | −16.596 | 14.290 | −1.171 | 1.00 | 70.79 | C |
| ATOM | 9432 | O | LYS | C | 277 | −17.212 | 13.485 | −1.873 | 1.00 | 70.79 | C |
| ATOM | 9433 | N | ARG | C | 278 | −16.442 | 15.575 | −1.489 | 1.00 | 69.75 | C |
| ATOM | 9434 | CA | ARG | C | 278 | −17.000 | 16.169 | −2.703 | 1.00 | 69.75 | C |
| ATOM | 9435 | CB | ARG | C | 278 | −18.505 | 16.371 | −2.535 | 1.00 | 170.83 | C |
| ATOM | 9436 | CG | ARG | C | 278 | −18.838 | 17.467 | −1.555 | 1.00 | 155.46 | C |
| ATOM | 9437 | CD | ARG | C | 278 | −20.301 | 17.474 | −1.198 | 1.00 | 155.46 | C |
| ATOM | 9438 | NE | ARG | C | 278 | −20.626 | 18.600 | −0.327 | 1.00 | 155.46 | C |
| ATOM | 9439 | CZ | ARG | C | 278 | −21.738 | 18.688 | 0.395 | 1.00 | 155.46 | C |
| ATOM | 9440 | NH1 | ARG | C | 278 | −22.634 | 17.709 | 0.352 | 1.00 | 155.46 | C |
| ATOM | 9441 | NH2 | ARG | C | 278 | −21.967 | 19.764 | 1.138 | 1.00 | 155.46 | C |
| ATOM | 9442 | C | ARG | C | 278 | −16.326 | 17.526 | −2.945 | 1.00 | 69.75 | C |
| ATOM | 9443 | O | ARG | C | 278 | −15.618 | 18.039 | −2.081 | 1.00 | 69.75 | C |
| ATOM | 9444 | N | PHE | C | 279 | −16.535 | 18.103 | −4.120 | 1.00 | 59.88 | C |
| ATOM | 9445 | CA | PHE | C | 279 | −15.958 | 19.404 | −4.420 | 1.00 | 59.88 | C |
| ATOM | 9446 | CB | PHE | C | 279 | −15.695 | 19.567 | −5.911 | 1.00 | 64.92 | C |
| ATOM | 9447 | CG | PHE | C | 279 | −14.453 | 18.893 | −6.385 | 1.00 | 48.69 | C |
| ATOM | 9448 | CD1 | PHE | C | 279 | −14.485 | 17.591 | −6.857 | 1.00 | 48.69 | C |
| ATOM | 9449 | CD2 | PHE | C | 279 | −13.244 | 19.562 | −6.365 | 1.00 | 48.69 | C |
| ATOM | 9450 | CE1 | PHE | C | 279 | −13.320 | 16.959 | −7.310 | 1.00 | 48.69 | C |
| ATOM | 9451 | CE2 | PHE | C | 279 | −12.076 | 18.944 | −6.811 | 1.00 | 48.69 | C |
| ATOM | 9452 | CZ | PHE | C | 279 | −12.114 | 17.637 | −7.285 | 1.00 | 48.69 | C |
| ATOM | 9453 | C | PHE | C | 279 | −16.898 | 20.512 | −3.981 | 1.00 | 59.88 | C |
| ATOM | 9454 | O | PHE | C | 279 | −18.121 | 20.320 | −3.902 | 1.00 | 59.88 | C |
| ATOM | 9455 | N | THR | C | 280 | −16.324 | 21.677 | −3.697 | 1.00 | 59.07 | C |
| ATOM | 9456 | CA | THR | C | 280 | −17.110 | 22.830 | −3.289 | 1.00 | 59.07 | C |
| ATOM | 9457 | CB | THR | C | 280 | −16.990 | 23.087 | −1.796 | 1.00 | 79.48 | C |
| ATOM | 9458 | OG1 | THR | C | 280 | −17.248 | 21.872 | −1.086 | 1.00 | 60.00 | C |
| ATOM | 9459 | CG2 | THR | C | 280 | −17.995 | 24.143 | −1.370 | 1.00 | 60.00 | C |
| ATOM | 9460 | C | THR | C | 280 | −16.623 | 24.068 | −4.029 | 1.00 | 59.07 | C |
| ATOM | 9461 | O | THR | C | 280 | −15.420 | 24.257 | −4.216 | 1.00 | 59.07 | C |
| ATOM | 9462 | N | LYS | C | 281 | −17.566 | 24.899 | −4.458 | 1.00 | 62.32 | C |
| ATOM | 9463 | CA | LYS | C | 281 | −17.251 | 26.130 | −5.172 | 1.00 | 62.32 | C |
| ATOM | 9464 | CB | LYS | C | 281 | −18.530 | 26.952 | −5.349 | 1.00 | 110.69 | C |
| ATOM | 9465 | CG | LYS | C | 281 | −19.471 | 26.863 | −4.145 | 1.00 | 86.63 | C |
| ATOM | 9466 | CD | LYS | C | 281 | −19.942 | 28.232 | −3.660 | 1.00 | 86.63 | C |
| ATOM | 9467 | CE | LYS | C | 281 | −20.809 | 28.946 | −4.685 | 1.00 | 86.63 | C |
| ATOM | 9468 | NZ | LYS | C | 281 | −21.313 | 30.251 | −4.166 | 1.00 | 86.63 | C |
| ATOM | 9469 | C | LYS | C | 281 | −16.207 | 26.966 | −4.425 | 1.00 | 62.32 | C |
| ATOM | 9470 | O | LYS | C | 281 | −16.224 | 27.050 | −3.195 | 1.00 | 62.32 | C |
| ATOM | 9471 | N | MET | C | 282 | −15.289 | 27.572 | −5.171 | 1.00 | 71.11 | C |
| ATOM | 9472 | CA | MET | C | 282 | −14.269 | 28.427 | −4.578 | 1.00 | 71.11 | C |
| ATOM | 9473 | CB | MET | C | 282 | −12.896 | 28.138 | −5.170 | 1.00 | 67.76 | C |
| ATOM | 9474 | CG | MET | C | 282 | −12.308 | 26.821 | −4.773 | 1.00 | 67.76 | C |
| ATOM | 9475 | SD | MET | C | 282 | −10.546 | 26.795 | −5.136 | 1.00 | 67.76 | C |
| ATOM | 9476 | CE | MET | C | 282 | −10.535 | 26.107 | −6.783 | 1.00 | 67.76 | C |
| ATOM | 9477 | C | MET | C | 282 | −14.624 | 29.877 | −4.868 | 1.00 | 71.11 | C |
| ATOM | 9478 | O | MET | C | 282 | −14.005 | 30.792 | −4.330 | 1.00 | 71.11 | C |
| ATOM | 9479 | N | THR | C | 283 | −15.623 | 30.071 | −5.726 | 1.00 | 92.59 | C |
| ATOM | 9480 | CA | THR | C | 283 | −16.084 | 31.395 | −6.131 | 1.00 | 92.59 | C |
| ATOM | 9481 | CB | THR | C | 283 | −15.625 | 31.718 | −7.571 | 1.00 | 80.53 | C |
| ATOM | 9482 | OG1 | THR | C | 283 | −14.196 | 31.779 | −7.619 | 1.00 | 80.53 | C |
| ATOM | 9483 | CG2 | THR | C | 283 | −16.192 | 33.043 | −8.035 | 1.00 | 80.53 | C |
| ATOM | 9484 | C | THR | C | 283 | −17.605 | 31.413 | −6.100 | 1.00 | 92.59 | C |
| ATOM | 9485 | O | THR | C | 283 | −18.236 | 30.359 | −6.149 | 1.00 | 92.59 | C |
| ATOM | 9486 | N | HIS | C | 284 | −18.187 | 32.608 | −6.010 | 1.00 | 86.39 | C |
| ATOM | 9487 | CA | HIS | C | 284 | −19.644 | 32.764 | −5.994 | 1.00 | 86.39 | C |
| ATOM | 9488 | CB | HIS | C | 284 | −20.008 | 34.251 | −5.914 | 1.00 | 163.30 | C |
| ATOM | 9489 | CG | HIS | C | 284 | −19.408 | 35.076 | −7.012 | 1.00 | 163.30 | C |
| ATOM | 9490 | CD2 | HIS | C | 284 | −19.983 | 35.767 | −8.026 | 1.00 | 163.30 | C |
| ATOM | 9491 | ND1 | HIS | C | 284 | −18.047 | 35.236 | −7.166 | 1.00 | 163.30 | C |
| ATOM | 9492 | CE1 | HIS | C | 284 | −17.809 | 35.987 | −8.227 | 1.00 | 163.30 | C |
| ATOM | 9493 | NE2 | HIS | C | 284 | −18.967 | 36.322 | −8.766 | 1.00 | 163.30 | C |
| ATOM | 9494 | C | HIS | C | 284 | −20.207 | 32.167 | −7.287 | 1.00 | 86.39 | C |
| ATOM | 9495 | O | HIS | C | 284 | −21.284 | 31.569 | −7.303 | 1.00 | 86.39 | C |
| ATOM | 9496 | N | LYS | C | 285 | −19.437 | 32.331 | −8.360 | 1.00 | 110.69 | C |
| ATOM | 9497 | CA | LYS | C | 285 | −19.787 | 31.855 | −9.693 | 1.00 | 110.69 | C |
| ATOM | 9498 | CB | LYS | C | 285 | −18.593 | 32.054 | −10.635 | 1.00 | 105.97 | C |
| ATOM | 9499 | CG | LYS | C | 285 | −18.926 | 32.774 | −11.931 | 1.00 | 105.97 | C |
| ATOM | 9500 | CD | LYS | C | 285 | −19.374 | 34.212 | −11.671 | 1.00 | 105.97 | C |
| ATOM | 9501 | CE | LYS | C | 285 | −19.695 | 34.946 | −12.972 | 1.00 | 105.97 | C |
| ATOM | 9502 | NZ | LYS | C | 285 | −20.113 | 36.355 | −12.736 | 1.00 | 105.97 | C |
| ATOM | 9503 | C | LYS | C | 285 | −20.230 | 30.396 | −9.760 | 1.00 | 110.69 | C |
| ATOM | 9504 | O | LYS | C | 285 | −21.049 | 30.036 | −10.604 | 1.00 | 110.69 | C |
| ATOM | 9505 | N | GLY | C | 286 | −19.690 | 29.562 | −8.876 | 1.00 | 77.06 | C |
| ATOM | 9506 | CA | GLY | C | 286 | −20.029 | 28.146 | −8.880 | 1.00 | 77.06 | C |
| ATOM | 9507 | C | GLY | C | 286 | −19.145 | 27.400 | −9.865 | 1.00 | 77.06 | C |
| ATOM | 9508 | O | GLY | C | 286 | −18.247 | 27.993 | −10.466 | 1.00 | 77.06 | C |
| ATOM | 9509 | N | PHE | C | 287 | −19.377 | 26.104 | −10.035 | 1.00 | 83.65 | C |
| ATOM | 9510 | CA | PHE | C | 287 | −18.577 | 25.323 | −10.974 | 1.00 | 83.65 | C |
| ATOM | 9511 | CB | PHE | C | 287 | −17.403 | 24.644 | −10.255 | 1.00 | 67.85 | C |
| ATOM | 9512 | CG | PHE | C | 287 | −17.814 | 23.552 | −9.307 | 1.00 | 67.85 | C |
| ATOM | 9513 | CD1 | PHE | C | 287 | −18.013 | 22.249 | −9.765 | 1.00 | 67.85 | C |
| ATOM | 9514 | CD2 | PHE | C | 287 | −18.009 | 23.826 | −7.951 | 1.00 | 67.85 | C |
| ATOM | 9515 | CE1 | PHE | C | 287 | −18.403 | 21.226 | −8.885 | 1.00 | 67.85 | C |
| ATOM | 9516 | CE2 | PHE | C | 287 | −18.397 | 22.818 | −7.059 | 1.00 | 67.85 | C |
| ATOM | 9517 | CZ | PHE | C | 287 | −18.594 | 21.513 | −7.528 | 1.00 | 67.85 | C |
| ATOM | 9518 | C | PHE | C | 287 | −19.434 | 24.279 | −11.673 | 1.00 | 83.65 | C |
| ATOM | 9519 | O | PHE | C | 287 | −20.327 | 23.688 | −11.062 | 1.00 | 83.65 | C |
| ATOM | 9520 | N | PRO | C | 288 | −19.176 | 24.042 | −12.973 | 1.00 | 83.71 | C |
| ATOM | 9521 | CD | PRO | C | 288 | −18.098 | 24.648 | −13.777 | 1.00 | 83.47 | C |
| ATOM | 9522 | CA | PRO | C | 288 | −19.926 | 23.063 | −13.768 | 1.00 | 83.71 | C |
| ATOM | 9523 | CB | PRO | C | 288 | −19.393 | 23.289 | −15.178 | 1.00 | 83.47 | C |
| ATOM | 9524 | CG | PRO | C | 288 | −17.971 | 23.676 | −14.931 | 1.00 | 83.47 | C |
| ATOM | 9525 | C | PRO | C | 288 | −19.713 | 21.629 | −13.291 | 1.00 | 83.71 | C |
| ATOM | 9526 | O | PRO | C | 288 | −18.583 | 21.141 | −13.234 | 1.00 | 83.71 | C |
| ATOM | 9527 | N | LYS | C | 289 | −20.805 | 20.955 | −12.947 | 1.00 | 83.75 | C |
| ATOM | 9528 | CA | LYS | C | 289 | −20.713 | 19.583 | −12.477 | 1.00 | 83.75 | C |
| ATOM | 9529 | CB | LYS | C | 289 | −21.943 | 19.232 | −11.629 | 1.00 | 115.54 | C |
| ATOM | 9530 | CG | LYS | C | 289 | −21.865 | 19.818 | −10.222 | 1.00 | 115.54 | C |
| ATOM | 9531 | CD | LYS | C | 289 | −23.080 | 19.479 | −9.389 | 1.00 | 115.54 | C |
| ATOM | 9532 | CE | LYS | C | 289 | −24.301 | 20.231 | −9.886 | 1.00 | 115.54 | C |
| ATOM | 9533 | NZ | LYS | C | 289 | −25.513 | 19.938 | −9.067 | 1.00 | 115.54 | C |
| ATOM | 9534 | C | LYS | C | 289 | −20.545 | 18.619 | −13.639 | 1.00 | 83.75 | C |
| ATOM | 9535 | O | LYS | C | 289 | −21.493 | 17.955 | −14.061 | 1.00 | 83.75 | C |
| ATOM | 9536 | N | VAL | C | 290 | −19.314 | 18.551 | −14.140 | 1.00 | 73.24 | C |
| ATOM | 9537 | CA | VAL | C | 290 | −18.969 | 17.690 | −15.265 | 1.00 | 73.24 | C |
| ATOM | 9538 | CB | VAL | C | 290 | −18.854 | 18.516 | −16.567 | 1.00 | 96.18 | C |
| ATOM | 9539 | CG1 | VAL | C | 290 | −20.108 | 19.350 | −16.769 | 1.00 | 96.18 | C |
| ATOM | 9540 | CG2 | VAL | C | 290 | −17.624 | 19.419 | −16.504 | 1.00 | 96.18 | C |
| ATOM | 9541 | C | VAL | C | 290 | −17.625 | 17.013 | −15.008 | 1.00 | 73.24 | C |
| ATOM | 9542 | O | VAL | C | 290 | −16.914 | 17.372 | −14.076 | 1.00 | 73.24 | C |
| ATOM | 9543 | N | GLY | C | 291 | −17.281 | 16.040 | −15.843 | 1.00 | 75.93 | C |
| ATOM | 9544 | CA | GLY | C | 291 | −16.012 | 15.344 | −15.700 | 1.00 | 75.93 | C |
| ATOM | 9545 | C | GLY | C | 291 | −15.665 | 14.877 | −14.298 | 1.00 | 75.93 | C |
| ATOM | 9546 | O | GLY | C | 291 | −16.489 | 14.286 | −13.601 | 1.00 | 75.93 | C |
| ATOM | 9547 | N | MET | C | 292 | −14.431 | 15.143 | −13.884 | 1.00 | 75.26 | C |
| ATOM | 9548 | CA | MET | C | 292 | −13.941 | 14.748 | −12.565 | 1.00 | 75.26 | C |
| ATOM | 9549 | CB | MET | C | 292 | −12.444 | 14.975 | −12.492 | 1.00 | 91.77 | C |
| ATOM | 9550 | CG | MET | C | 292 | −12.110 | 16.455 | −12.492 | 1.00 | 91.77 | C |
| ATOM | 9551 | SD | MET | C | 292 | −10.434 | 16.803 | −11.939 | 1.00 | 91.77 | C |
| ATOM | 9552 | CE | MET | C | 292 | −9.726 | 17.466 | −13.413 | 1.00 | 91.77 | C |
| ATOM | 9553 | C | MET | C | 292 | −14.583 | 15.548 | −11.433 | 1.00 | 75.26 | C |
| ATOM | 9554 | O | MET | C | 292 | −14.269 | 15.337 | −10.267 | 1.00 | 75.26 | C |
| ATOM | 9555 | N | LEU | C | 293 | −15.475 | 16.468 | −11.767 | 1.00 | 72.28 | C |
| ATOM | 9556 | CA | LEU | C | 293 | −16.096 | 17.294 | −10.744 | 1.00 | 72.28 | C |
| ATOM | 9557 | CB | LEU | C | 293 | −16.230 | 18.730 | −11.262 | 1.00 | 79.11 | C |
| ATOM | 9558 | CG | LEU | C | 293 | −14.910 | 19.444 | −11.555 | 1.00 | 79.11 | C |
| ATOM | 9559 | CD1 | LEU | C | 293 | −15.166 | 20.802 | −12.201 | 1.00 | 79.11 | C |
| ATOM | 9560 | CD2 | LEU | C | 293 | −14.149 | 19.599 | −10.255 | 1.00 | 79.11 | C |
| ATOM | 9561 | C | LEU | C | 293 | −17.438 | 16.835 | −10.183 | 1.00 | 72.28 | C |
| ATOM | 9562 | O | LEU | C | 293 | −17.957 | 17.469 | −9.270 | 1.00 | 72.28 | C |
| ATOM | 9563 | N | THR | C | 294 | −18.012 | 15.756 | −10.708 | 1.00 | 88.44 | C |
| ATOM | 9564 | CA | THR | C | 294 | −19.301 | 15.291 | −10.188 | 1.00 | 88.44 | C |
| ATOM | 9565 | CB | THR | C | 294 | −20.441 | 15.386 | −11.217 | 1.00 | 106.22 | C |
| ATOM | 9566 | OG1 | THR | C | 294 | −20.321 | 16.599 | −11.963 | 1.00 | 106.22 | C |
| ATOM | 9567 | CG2 | THR | C | 294 | −21.790 | 15.374 | −10.502 | 1.00 | 106.22 | C |
| ATOM | 9568 | C | THR | C | 294 | −19.261 | 13.847 | −9.759 | 1.00 | 88.44 | C |
| ATOM | 9569 | O | THR | C | 294 | −18.496 | 13.043 | −10.289 | 1.00 | 88.44 | C |
| ATOM | 9570 | N | VAL | C | 295 | −20.110 | 13.520 | −8.799 | 1.00 | 85.62 | C |
| ATOM | 9571 | CA | VAL | C | 295 | −20.196 | 12.156 | −8.316 | 1.00 | 85.62 | C |
| ATOM | 9572 | CB | VAL | C | 295 | −21.076 | 12.088 | −7.060 | 1.00 | 104.14 | C |
| ATOM | 9573 | CG1 | VAL | C | 295 | −21.087 | 10.665 | −6.504 | 1.00 | 104.14 | C |
| ATOM | 9574 | CG2 | VAL | C | 295 | −20.563 | 13.087 | −6.024 | 1.00 | 104.14 | C |
| ATOM | 9575 | C | VAL | C | 295 | −20.812 | 11.302 | −9.426 | 1.00 | 85.62 | C |
| ATOM | 9576 | O | VAL | C | 295 | −20.655 | 10.080 | −9.447 | 1.00 | 85.62 | C |
| ATOM | 9577 | N | LYS | C | 296 | −21.504 | 11.969 | −10.348 | 1.00 | 92.07 | C |
| ATOM | 9578 | CA | LYS | C | 296 | −22.156 | 11.314 | −11.476 | 1.00 | 92.07 | C |
| ATOM | 9579 | CB | LYS | C | 296 | −22.863 | 12.353 | −12.351 | 1.00 | 95.69 | C |
| ATOM | 9580 | CG | LYS | C | 296 | −23.928 | 13.170 | −11.636 | 1.00 | 95.69 | C |
| ATOM | 9581 | CD | LYS | C | 296 | −25.183 | 12.360 | −11.369 | 1.00 | 95.69 | C |
| ATOM | 9582 | CE | LYS | C | 296 | −25.900 | 12.021 | −12.666 | 1.00 | 95.69 | C |
| ATOM | 9583 | NZ | LYS | C | 296 | −27.130 | 11.181 | −12.495 | 1.00 | 95.69 | C |
| ATOM | 9584 | C | LYS | C | 296 | −21.152 | 10.548 | −12.332 | 1.00 | 92.07 | C |
| ATOM | 9585 | O | LYS | C | 296 | −21.485 | 9.511 | −12.913 | 1.00 | 92.07 | C |
| ATOM | 9586 | N | ASN | C | 297 | −19.925 | 11.059 | −12.401 | 1.00 | 64.89 | C |
| ATOM | 9587 | CA | ASN | C | 297 | −18.874 | 10.442 | −13.207 | 1.00 | 64.89 | C |
| ATOM | 9588 | CB | ASN | C | 297 | −17.606 | 11.302 | −13.183 | 1.00 | 75.86 | C |
| ATOM | 9589 | CG | ASN | C | 297 | −16.571 | 10.856 | −14.211 | 1.00 | 75.86 | C |
| ATOM | 9590 | OD1 | ASN | C | 297 | −16.055 | 9.740 | −14.155 | 1.00 | 75.86 | C |
| ATOM | 9591 | ND2 | ASN | C | 297 | −16.266 | 11.734 | −15.158 | 1.00 | 75.86 | C |
| ATOM | 9592 | C | ASN | C | 297 | −18.526 | 9.015 | −12.793 | 1.00 | 64.89 | C |
| ATOM | 9593 | O | ASN | C | 297 | −18.387 | 8.695 | −11.612 | 1.00 | 64.89 | C |
| ATOM | 9594 | N | THR | C | 298 | −18.383 | 8.160 | −13.793 | 1.00 | 68.02 | C |
| ATOM | 9595 | CA | THR | C | 298 | −18.053 | 6.766 | −13.581 | 1.00 | 68.02 | C |
| ATOM | 9596 | CB | THR | C | 298 | −18.040 | 6.023 | −14.935 | 1.00 | 120.22 | C |
| ATOM | 9597 | OG1 | THR | C | 298 | −17.297 | 4.808 | −14.809 | 1.00 | 120.22 | C |
| ATOM | 9598 | CG2 | THR | C | 298 | −17.420 | 6.892 | −16.020 | 1.00 | 120.22 | C |
| ATOM | 9599 | C | THR | C | 298 | −16.713 | 6.546 | −12.854 | 1.00 | 68.02 | C |
| ATOM | 9600 | O | THR | C | 298 | −16.520 | 5.524 | −12.192 | 1.00 | 68.02 | C |
| ATOM | 9601 | N | LEU | C | 299 | −15.794 | 7.502 | −12.961 | 1.00 | 65.19 | C |
| ATOM | 9602 | CA | LEU | C | 299 | −14.489 | 7.369 | −12.313 | 1.00 | 65.19 | C |
| ATOM | 9603 | CB | LEU | C | 299 | −13.392 | 7.871 | −13.244 | 1.00 | 65.29 | C |
| ATOM | 9604 | CG | LEU | C | 299 | −13.211 | 7.041 | −14.503 | 1.00 | 65.29 | C |
| ATOM | 9605 | CD1 | LEU | C | 299 | −12.201 | 7.708 | −15.405 | 1.00 | 65.29 | C |
| ATOM | 9606 | CD2 | LEU | C | 299 | −12.761 | 5.653 | −14.125 | 1.00 | 65.29 | C |
| ATOM | 9607 | C | LEU | C | 299 | −14.378 | 8.093 | −10.969 | 1.00 | 65.19 | C |
| ATOM | 9608 | O | LEU | C | 299 | −13.299 | 8.151 | −10.370 | 1.00 | 65.19 | C |
| ATOM | 9609 | N | TRP | C | 300 | −15.494 | 8.642 | −10.499 | 1.00 | 51.07 | C |
| ATOM | 9610 | CA | TRP | C | 300 | −15.516 | 9.351 | −9.235 | 1.00 | 51.07 | C |
| ATOM | 9611 | CB | TRP | C | 300 | −16.954 | 9.632 | −8.808 | 1.00 | 75.13 | C |
| ATOM | 9612 | CG | TRP | C | 300 | −17.032 | 10.398 | −7.541 | 1.00 | 75.13 | C |
| ATOM | 9613 | CD2 | TRP | C | 300 | −16.494 | 11.702 | −7.297 | 1.00 | 75.13 | C |
| ATOM | 9614 | CE2 | TRP | C | 300 | −16.813 | 12.041 | −5.966 | 1.00 | 75.13 | C |
| ATOM | 9615 | CE3 | TRP | C | 300 | −15.773 | 12.620 | −8.075 | 1.00 | 75.13 | C |
| ATOM | 9616 | CD1 | TRP | C | 300 | −17.633 | 10.007 | −6.387 | 1.00 | 75.13 | C |
| ATOM | 9617 | NE1 | TRP | C | 300 | −17.508 | 10.987 | −5.434 | 1.00 | 75.13 | C |
| ATOM | 9618 | CZ2 | TRP | C | 300 | −16.438 | 13.264 | −5.390 | 1.00 | 75.13 | C |
| ATOM | 9619 | CZ3 | TRP | C | 300 | −15.398 | 13.836 | −7.505 | 1.00 | 75.13 | C |
| ATOM | 9620 | CH2 | TRP | C | 300 | −15.733 | 14.146 | −6.174 | 1.00 | 75.13 | C |
| ATOM | 9621 | C | TRP | C | 300 | −14.813 | 8.577 | −8.133 | 1.00 | 51.07 | C |
| ATOM | 9622 | O | TRP | C | 300 | −14.060 | 9.156 | −7.359 | 1.00 | 51.07 | C |
| ATOM | 9623 | N | LYS | C | 301 | −15.054 | 7.273 | −8.058 | 1.00 | 75.80 | C |
| ATOM | 9624 | CA | LYS | C | 301 | −14.431 | 6.464 | −7.018 | 1.00 | 75.80 | C |
| ATOM | 9625 | CB | LYS | C | 301 | −14.849 | 4.995 | −7.142 | 1.00 | 96.96 | C |
| ATOM | 9626 | CG | LYS | C | 301 | −16.359 | 4.775 | −7.029 | 1.00 | 96.96 | C |
| ATOM | 9627 | CD | LYS | C | 301 | −16.956 | 5.497 | −5.814 | 1.00 | 96.96 | C |
| ATOM | 9628 | CE | LYS | C | 301 | −18.474 | 5.631 | −5.927 | 1.00 | 96.96 | C |
| ATOM | 9629 | NZ | LYS | C | 301 | −19.037 | 6.650 | −4.991 | 1.00 | 96.96 | C |
| ATOM | 9630 | C | LYS | C | 301 | −12.914 | 6.586 | −7.046 | 1.00 | 75.80 | C |
| ATOM | 9631 | O | LYS | C | 301 | −12.265 | 6.593 | −5.997 | 1.00 | 75.80 | C |
| ATOM | 9632 | N | ASP | C | 302 | −12.342 | 6.692 | −8.240 | 1.00 | 70.07 | C |
| ATOM | 9633 | CA | ASP | C | 302 | −10.892 | 6.832 | −8.354 | 1.00 | 70.07 | C |
| ATOM | 9634 | CB | ASP | C | 302 | −10.439 | 6.565 | −9.789 | 1.00 | 95.43 | C |
| ATOM | 9635 | CG | ASP | C | 302 | −10.535 | 5.106 | −10.159 | 1.00 | 95.43 | C |
| ATOM | 9636 | OD1 | ASP | C | 302 | −10.015 | 4.276 | −9.383 | 1.00 | 95.43 | C |
| ATOM | 9637 | OD2 | ASP | C | 302 | −11.119 | 4.789 | −11.221 | 1.00 | 95.43 | C |
| ATOM | 9638 | C | ASP | C | 302 | −10.448 | 8.228 | −7.914 | 1.00 | 70.07 | C |
| ATOM | 9639 | O | ASP | C | 302 | −9.380 | 8.391 | −7.318 | 1.00 | 70.07 | C |
| ATOM | 9640 | N | VAL | C | 303 | −11.270 | 9.233 | −8.214 | 1.00 | 55.06 | C |
| ATOM | 9641 | CA | VAL | C | 303 | −10.963 | 10.600 | −7.817 | 1.00 | 55.06 | C |
| ATOM | 9642 | CB | VAL | C | 303 | −12.045 | 11.583 | −8.294 | 1.00 | 68.87 | C |
| ATOM | 9643 | CG1 | VAL | C | 303 | −11.784 | 12.962 | −7.731 | 1.00 | 58.18 | C |
| ATOM | 9644 | CG2 | VAL | C | 303 | −12.053 | 11.637 | −9.806 | 1.00 | 58.18 | C |
| ATOM | 9645 | C | VAL | C | 303 | −10.883 | 10.635 | −6.297 | 1.00 | 55.06 | C |
| ATOM | 9646 | O | VAL | C | 303 | −9.882 | 11.083 | −5.743 | 1.00 | 55.06 | C |
| ATOM | 9647 | N | LYS | C | 304 | −11.931 | 10.148 | −5.631 | 1.00 | 67.86 | C |
| ATOM | 9648 | CA | LYS | C | 304 | −11.972 | 10.102 | −4.167 | 1.00 | 67.86 | C |
| ATOM | 9649 | CB | LYS | C | 304 | −13.170 | 9.292 | −3.681 | 1.00 | 92.69 | C |
| ATOM | 9650 | CG | LYS | C | 304 | −14.472 | 10.042 | −3.593 | 1.00 | 74.61 | C |
| ATOM | 9651 | CD | LYS | C | 304 | −15.566 | 9.100 | −3.117 | 1.00 | 74.61 | C |
| ATOM | 9652 | CE | LYS | C | 304 | −16.891 | 9.824 | −2.913 | 1.00 | 74.61 | C |
| ATOM | 9653 | NZ | LYS | C | 304 | −18.005 | 8.888 | −2.595 | 1.00 | 74.61 | C |
| ATOM | 9654 | C | LYS | C | 304 | −10.716 | 9.456 | −3.602 | 1.00 | 67.86 | C |
| ATOM | 9655 | O | LYS | C | 304 | −10.116 | 9.958 | −2.652 | 1.00 | 67.86 | C |
| ATOM | 9656 | N | ALA | C | 305 | −10.329 | 8.333 | −4.194 | 1.00 | 65.11 | C |
| ATOM | 9657 | CA | ALA | C | 305 | −9.155 | 7.594 | −3.748 | 1.00 | 65.11 | C |
| ATOM | 9658 | CB | ALA | C | 305 | −9.174 | 6.191 | −4.358 | 1.00 | 37.36 | C |
| ATOM | 9659 | C | ALA | C | 305 | −7.812 | 8.275 | −4.034 | 1.00 | 65.11 | C |
| ATOM | 9660 | O | ALA | C | 305 | −6.983 | 8.410 | −3.141 | 1.00 | 65.11 | C |
| ATOM | 9661 | N | TYR | C | 306 | −7.592 | 8.702 | −5.271 | 1.00 | 51.00 | C |
| ATOM | 9662 | CA | TYR | C | 306 | −6.328 | 9.344 | −5.629 | 1.00 | 51.00 | C |
| ATOM | 9663 | CB | TYR | C | 306 | −6.177 | 9.401 | −7.144 | 1.00 | 83.58 | C |
| ATOM | 9664 | CG | TYR | C | 306 | −6.017 | 8.046 | −7.779 | 1.00 | 78.27 | C |
| ATOM | 9665 | CD1 | TYR | C | 306 | −6.468 | 7.807 | −9.074 | 1.00 | 78.27 | C |
| ATOM | 9666 | CE1 | TYR | C | 306 | −6.328 | 6.558 | −9.661 | 1.00 | 78.27 | C |
| ATOM | 9667 | CD2 | TYR | C | 306 | −5.416 | 7.000 | −7.087 | 1.00 | 78.27 | C |
| ATOM | 9668 | CE2 | TYR | C | 306 | −5.271 | 5.755 | −7.665 | 1.00 | 78.27 | C |
| ATOM | 9669 | CZ | TYR | C | 306 | −5.728 | 5.538 | −8.950 | 1.00 | 78.27 | C |
| ATOM | 9670 | OH | TYR | C | 306 | −5.578 | 4.301 | −9.527 | 1.00 | 78.27 | C |
| ATOM | 9671 | C | TYR | C | 306 | −6.162 | 10.738 | −5.070 | 1.00 | 51.00 | C |
| ATOM | 9672 | O | TYR | C | 306 | −5.127 | 11.041 | −4.491 | 1.00 | 51.00 | C |
| ATOM | 9673 | N | LEU | C | 307 | −7.173 | 11.585 | −5.256 | 1.00 | 48.75 | C |
| ATOM | 9674 | CA | LEU | C | 307 | −7.118 | 12.958 | −4.767 | 1.00 | 48.75 | C |
| ATOM | 9675 | CB | LEU | C | 307 | −8.055 | 13.853 | −5.580 | 1.00 | 71.28 | C |
| ATOM | 9676 | CG | LEU | C | 307 | −8.211 | 15.291 | −5.070 | 1.00 | 39.41 | C |
| ATOM | 9677 | CD1 | LEU | C | 307 | −6.864 | 16.014 | −5.066 | 1.00 | 39.41 | C |
| ATOM | 9678 | CD2 | LEU | C | 307 | −9.216 | 16.022 | −5.945 | 1.00 | 39.41 | C |
| ATOM | 9679 | C | LEU | C | 307 | −7.477 | 13.050 | −3.288 | 1.00 | 48.75 | C |
| ATOM | 9680 | O | LEU | C | 307 | −6.723 | 13.617 | −2.491 | 1.00 | 48.75 | C |
| ATOM | 9681 | N | GLY | C | 308 | −8.627 | 12.493 | −2.926 | 1.00 | 65.44 | C |
| ATOM | 9682 | CA | GLY | C | 308 | −9.052 | 12.522 | −1.539 | 1.00 | 65.44 | C |
| ATOM | 9683 | C | GLY | C | 308 | −8.048 | 11.857 | −0.611 | 1.00 | 65.44 | C |
| ATOM | 9684 | O | GLY | C | 308 | −7.702 | 12.401 | 0.433 | 1.00 | 65.44 | C |
| ATOM | 9685 | N | GLY | C | 309 | −7.574 | 10.678 | −0.989 | 1.00 | 57.47 | C |
| ATOM | 9686 | CA | GLY | C | 309 | −6.622 | 9.972 | −0.153 | 1.00 | 57.47 | C |
| ATOM | 9687 | C | GLY | C | 309 | −5.341 | 10.742 | 0.095 | 1.00 | 57.47 | C |
| ATOM | 9688 | O | GLY | C | 309 | −4.877 | 10.867 | 1.236 | 1.00 | 57.47 | C |
| ATOM | 9689 | N | PHE | C | 310 | −4.763 | 11.257 | −0.984 | 1.00 | 53.55 | C |
| ATOM | 9690 | CA | PHE | C | 310 | −3.523 | 12.012 | −0.906 | 1.00 | 53.55 | C |
| ATOM | 9691 | CB | PHE | C | 310 | −3.140 | 12.494 | −2.306 | 1.00 | 47.57 | C |
| ATOM | 9692 | CG | PHE | C | 310 | −1.755 | 13.070 | −2.398 | 1.00 | 43.40 | C |
| ATOM | 9693 | CD1 | PHE | C | 310 | −0.648 | 12.286 | −2.122 | 1.00 | 43.40 | C |
| ATOM | 9694 | CD2 | PHE | C | 310 | −1.562 | 14.399 | −2.784 | 1.00 | 43.40 | C |
| ATOM | 9695 | CE1 | PHE | C | 310 | 0.633 | 12.810 | −2.230 | 1.00 | 43.40 | C |
| ATOM | 9696 | CE2 | PHE | C | 310 | −0.287 | 14.933 | −2.895 | 1.00 | 43.40 | C |
| ATOM | 9697 | CZ | PHE | C | 310 | 0.815 | 14.135 | −2.617 | 1.00 | 43.40 | C |
| ATOM | 9698 | C | PHE | C | 310 | −3.710 | 13.202 | 0.028 | 1.00 | 53.55 | C |
| ATOM | 9699 | O | PHE | C | 310 | −2.870 | 13.477 | 0.889 | 1.00 | 53.55 | C |
| ATOM | 9700 | N | GLU | C | 311 | −4.828 | 13.896 | −0.151 | 1.00 | 44.24 | C |
| ATOM | 9701 | CA | GLU | C | 311 | −5.144 | 15.069 | 0.642 | 1.00 | 44.24 | C |
| ATOM | 9702 | CB | GLU | C | 311 | −6.325 | 15.817 | 0.032 | 1.00 | 85.30 | C |
| ATOM | 9703 | CG | GLU | C | 311 | −6.373 | 17.268 | 0.438 | 1.00 | 69.53 | C |
| ATOM | 9704 | CD | GLU | C | 311 | −7.626 | 17.977 | −0.030 | 1.00 | 69.53 | C |
| ATOM | 9705 | OE1 | GLU | C | 311 | −8.087 | 17.704 | −1.155 | 1.00 | 69.53 | C |
| ATOM | 9706 | OE2 | GLU | C | 311 | −8.141 | 18.825 | 0.725 | 1.00 | 69.53 | C |
| ATOM | 9707 | C | GLU | C | 311 | −5.474 | 14.703 | 2.080 | 1.00 | 44.24 | C |
| ATOM | 9708 | O | GLU | C | 311 | −5.176 | 15.450 | 3.001 | 1.00 | 44.24 | C |
| ATOM | 9709 | N | TYR | C | 312 | −6.093 | 13.548 | 2.276 | 1.00 | 47.09 | C |
| ATOM | 9710 | CA | TYR | C | 312 | −6.459 | 13.115 | 3.613 | 1.00 | 47.09 | C |
| ATOM | 9711 | CB | TYR | C | 312 | −7.407 | 11.917 | 3.557 | 1.00 | 52.61 | C |
| ATOM | 9712 | CG | TYR | C | 312 | −7.883 | 11.503 | 4.928 | 1.00 | 47.31 | C |
| ATOM | 9713 | CD1 | TYR | C | 312 | −8.802 | 12.281 | 5.632 | 1.00 | 47.31 | C |
| ATOM | 9714 | CE1 | TYR | C | 312 | −9.207 | 11.931 | 6.926 | 1.00 | 47.31 | C |
| ATOM | 9715 | CD2 | TYR | C | 312 | −7.379 | 10.359 | 5.549 | 1.00 | 47.31 | C |
| ATOM | 9716 | CE2 | TYR | C | 312 | −7.775 | 9.998 | 6.842 | 1.00 | 47.31 | C |
| ATOM | 9717 | CZ | TYR | C | 312 | −8.687 | 10.786 | 7.523 | 1.00 | 47.31 | C |
| ATOM | 9718 | OH | TYR | C | 312 | −9.070 | 10.443 | 8.801 | 1.00 | 47.31 | C |
| ATOM | 9719 | C | TYR | C | 312 | −5.270 | 12.746 | 4.484 | 1.00 | 47.09 | C |
| ATOM | 9720 | O | TYR | C | 312 | −5.272 | 13.019 | 5.676 | 1.00 | 47.09 | C |
| ATOM | 9721 | N | MET | C | 313 | −4.257 | 12.118 | 3.904 | 1.00 | 42.18 | C |
| ATOM | 9722 | CA | MET | C | 313 | −3.113 | 11.727 | 4.697 | 1.00 | 42.18 | C |
| ATOM | 9723 | CB | MET | C | 313 | −3.483 | 10.525 | 5.554 | 1.00 | 72.15 | C |
| ATOM | 9724 | CG | MET | C | 313 | −2.370 | 10.061 | 6.459 | 1.00 | 72.00 | C |
| ATOM | 9725 | SD | MET | C | 313 | −2.807 | 8.576 | 7.352 | 1.00 | 72.00 | C |
| ATOM | 9726 | CE | MET | C | 313 | −4.305 | 9.121 | 8.174 | 1.00 | 72.00 | C |
| ATOM | 9727 | C | MET | C | 313 | −1.881 | 11.393 | 3.869 | 1.00 | 42.18 | C |
| ATOM | 9728 | O | MET | C | 313 | −0.747 | 11.636 | 4.302 | 1.00 | 42.18 | C |
| ATOM | 9729 | N | GLY | C | 314 | −2.096 | 10.830 | 2.683 | 1.00 | 47.28 | C |
| ATOM | 9730 | CA | GLY | C | 314 | −0.977 | 10.453 | 1.835 | 1.00 | 47.28 | C |
| ATOM | 9731 | C | GLY | C | 314 | 0.124 | 11.489 | 1.718 | 1.00 | 47.28 | C |
| ATOM | 9732 | O | GLY | C | 314 | 1.301 | 11.199 | 1.945 | 1.00 | 47.28 | C |
| ATOM | 9733 | N | CYS | C | 315 | −0.265 | 12.707 | 1.370 | 1.00 | 53.88 | C |
| ATOM | 9734 | CA | CYS | C | 315 | 0.694 | 13.777 | 1.207 | 1.00 | 53.88 | C |
| ATOM | 9735 | CB | CYS | C | 315 | −0.019 | 15.040 | 0.738 | 1.00 | 72.48 | C |
| ATOM | 9736 | SG | CYS | C | 315 | 1.109 | 16.278 | 0.092 | 1.00 | 77.85 | C |
| ATOM | 9737 | C | CYS | C | 315 | 1.498 | 14.072 | 2.475 | 1.00 | 53.88 | C |
| ATOM | 9738 | O | CYS | C | 315 | 2.734 | 14.085 | 2.444 | 1.00 | 53.88 | C |
| ATOM | 9739 | N | SER | C | 316 | 0.805 | 14.304 | 3.587 | 1.00 | 53.13 | C |
| ATOM | 9740 | CA | SER | C | 316 | 1.483 | 14.604 | 4.847 | 1.00 | 53.13 | C |
| ATOM | 9741 | CB | SER | C | 316 | 0.469 | 14.754 | 5.971 | 1.00 | 46.10 | C |
| ATOM | 9742 | OG | SER | C | 316 | −0.116 | 13.500 | 6.272 | 1.00 | 51.60 | C |
| ATOM | 9743 | C | SER | C | 316 | 2.506 | 13.535 | 5.242 | 1.00 | 53.13 | C |
| ATOM | 9744 | O | SER | C | 316 | 3.458 | 13.821 | 5.966 | 1.00 | 53.13 | C |
| ATOM | 9745 | N | LEU | C | 317 | 2.303 | 12.301 | 4.791 | 1.00 | 52.52 | C |
| ATOM | 9746 | CA | LEU | C | 317 | 3.246 | 11.233 | 5.094 | 1.00 | 52.52 | C |
| ATOM | 9747 | CB | LEU | C | 317 | 2.562 | 9.869 | 4.987 | 1.00 | 60.26 | C |
| ATOM | 9748 | CG | LEU | C | 317 | 1.558 | 9.534 | 6.092 | 1.00 | 52.77 | C |
| ATOM | 9749 | CD1 | LEU | C | 317 | 0.845 | 8.231 | 5.776 | 1.00 | 52.77 | C |
| ATOM | 9750 | CD2 | LEU | C | 317 | 2.290 | 9.434 | 7.416 | 1.00 | 52.77 | C |
| ATOM | 9751 | C | LEU | C | 317 | 4.408 | 11.297 | 4.115 | 1.00 | 52.52 | C |
| ATOM | 9752 | O | LEU | C | 317 | 5.573 | 11.172 | 4.497 | 1.00 | 52.52 | C |
| ATOM | 9753 | N | ALA | C | 318 | 4.080 | 11.503 | 2.846 | 1.00 | 60.76 | C |
| ATOM | 9754 | CA | ALA | C | 318 | 5.084 | 11.572 | 1.804 | 1.00 | 60.76 | C |
| ATOM | 9755 | CB | ALA | C | 318 | 4.405 | 11.706 | 0.462 | 1.00 | 42.38 | C |
| ATOM | 9756 | C | ALA | C | 318 | 6.067 | 12.717 | 2.002 | 1.00 | 60.76 | C |
| ATOM | 9757 | O | ALA | C | 318 | 7.282 | 12.527 | 1.893 | 1.00 | 60.76 | C |
| ATOM | 9758 | N | ILE | C | 319 | 5.544 | 13.905 | 2.297 | 1.00 | 52.41 | C |
| ATOM | 9759 | CA | ILE | C | 319 | 6.400 | 15.072 | 2.465 | 1.00 | 52.41 | C |
| ATOM | 9760 | CB | ILE | C | 319 | 5.610 | 16.285 | 2.992 | 1.00 | 69.59 | C |
| ATOM | 9761 | CG2 | ILE | C | 319 | 5.252 | 16.078 | 4.439 | 1.00 | 44.15 | C |
| ATOM | 9762 | CG1 | ILE | C | 319 | 6.459 | 17.548 | 2.854 | 1.00 | 44.15 | C |
| ATOM | 9763 | CD1 | ILE | C | 319 | 5.736 | 18.838 | 3.205 | 1.00 | 44.15 | C |
| ATOM | 9764 | C | ILE | C | 319 | 7.577 | 14.798 | 3.390 | 1.00 | 52.41 | C |
| ATOM | 9765 | O | ILE | C | 319 | 8.641 | 15.388 | 3.230 | 1.00 | 52.41 | C |
| ATOM | 9766 | N | LEU | C | 320 | 7.398 | 13.897 | 4.350 | 1.00 | 62.98 | C |
| ATOM | 9767 | CA | LEU | C | 320 | 8.475 | 13.573 | 5.278 | 1.00 | 62.98 | C |
| ATOM | 9768 | CB | LEU | C | 320 | 7.922 | 12.823 | 6.490 | 1.00 | 75.20 | C |
| ATOM | 9769 | CG | LEU | C | 320 | 6.975 | 13.641 | 7.373 | 1.00 | 53.04 | C |
| ATOM | 9770 | CD1 | LEU | C | 320 | 6.545 | 12.804 | 8.557 | 1.00 | 53.04 | C |
| ATOM | 9771 | CD2 | LEU | C | 320 | 7.664 | 14.919 | 7.846 | 1.00 | 53.04 | C |
| ATOM | 9772 | C | LEU | C | 320 | 9.599 | 12.768 | 4.615 | 1.00 | 62.98 | C |
| ATOM | 9773 | O | LEU | C | 320 | 10.765 | 12.885 | 5.000 | 1.00 | 62.98 | C |
| ATOM | 9774 | N | ASP | C | 321 | 9.256 | 11.955 | 3.620 | 1.00 | 71.04 | C |
| ATOM | 9775 | CA | ASP | C | 321 | 10.264 | 11.167 | 2.913 | 1.00 | 71.04 | C |
| ATOM | 9776 | CB | ASP | C | 321 | 9.610 | 10.084 | 2.049 | 1.00 | 112.33 | C |
| ATOM | 9777 | CG | ASP | C | 321 | 8.998 | 8.964 | 2.875 | 1.00 | 91.33 | C |
| ATOM | 9778 | OD1 | ASP | C | 321 | 9.659 | 8.506 | 3.831 | 1.00 | 91.33 | C |
| ATOM | 9779 | OD2 | ASP | C | 321 | 7.866 | 8.533 | 2.560 | 1.00 | 91.33 | C |
| ATOM | 9780 | C | ASP | C | 321 | 11.096 | 12.093 | 2.037 | 1.00 | 71.04 | C |
| ATOM | 9781 | O | ASP | C | 321 | 12.307 | 11.915 | 1.921 | 1.00 | 71.04 | C |
| ATOM | 9782 | N | TYR | C | 322 | 10.446 | 13.079 | 1.421 | 1.00 | 69.39 | C |
| ATOM | 9783 | CA | TYR | C | 322 | 11.155 | 14.038 | 0.579 | 1.00 | 69.39 | C |
| ATOM | 9784 | CB | TYR | C | 322 | 10.199 | 15.066 | −0.029 | 1.00 | 68.89 | C |
| ATOM | 9785 | CG | TYR | C | 322 | 9.352 | 14.563 | −1.174 | 1.00 | 59.60 | C |
| ATOM | 9786 | CD1 | TYR | C | 322 | 8.301 | 13.680 | −0.953 | 1.00 | 59.60 | C |
| ATOM | 9787 | CE1 | TYR | C | 322 | 7.489 | 13.249 | −2.005 | 1.00 | 59.60 | C |
| ATOM | 9788 | CD2 | TYR | C | 322 | 9.580 | 15.002 | −2.479 | 1.00 | 59.60 | C |
| ATOM | 9789 | CE2 | TYR | C | 322 | 8.775 | 14.577 | −3.534 | 1.00 | 59.60 | C |
| ATOM | 9790 | CZ | TYR | C | 322 | 7.729 | 13.701 | −3.287 | 1.00 | 59.60 | C |
| ATOM | 9791 | OH | TYR | C | 322 | 6.906 | 13.286 | −4.310 | 1.00 | 59.60 | C |
| ATOM | 9792 | C | TYR | C | 322 | 12.167 | 14.778 | 1.437 | 1.00 | 69.39 | C |
| ATOM | 9793 | O | TYR | C | 322 | 13.328 | 14.918 | 1.064 | 1.00 | 69.39 | C |
| ATOM | 9794 | N | GLN | C | 323 | 11.711 | 15.250 | 2.591 | 1.00 | 59.76 | C |
| ATOM | 9795 | CA | GLN | C | 323 | 12.564 | 15.980 | 3.504 | 1.00 | 59.76 | C |
| ATOM | 9796 | CB | GLN | C | 323 | 11.812 | 16.295 | 4.785 | 1.00 | 88.66 | C |
| ATOM | 9797 | CG | GLN | C | 323 | 12.642 | 17.050 | 5.775 | 1.00 | 63.05 | C |
| ATOM | 9798 | CD | GLN | C | 323 | 11.851 | 17.461 | 6.995 | 1.00 | 63.05 | C |
| ATOM | 9799 | OE1 | GLN | C | 323 | 11.186 | 16.640 | 7.620 | 1.00 | 63.05 | C |
| ATOM | 9800 | NE2 | GLN | C | 323 | 11.924 | 18.738 | 7.346 | 1.00 | 63.05 | C |
| ATOM | 9801 | C | GLN | C | 323 | 13.827 | 15.201 | 3.821 | 1.00 | 59.76 | C |
| ATOM | 9802 | O | GLN | C | 323 | 14.920 | 15.762 | 3.791 | 1.00 | 59.76 | C |
| ATOM | 9803 | N | LYS | C | 324 | 13.685 | 13.913 | 4.121 | 1.00 | 63.12 | C |
| ATOM | 9804 | CA | LYS | C | 324 | 14.840 | 13.073 | 4.429 | 1.00 | 63.12 | C |
| ATOM | 9805 | CB | LYS | C | 324 | 14.413 | 11.631 | 4.702 | 1.00 | 100.10 | C |
| ATOM | 9806 | CG | LYS | C | 324 | 13.835 | 11.383 | 6.066 | 1.00 | 89.53 | C |
| ATOM | 9807 | CD | LYS | C | 324 | 13.711 | 9.887 | 6.314 | 1.00 | 89.53 | C |
| ATOM | 9808 | CE | LYS | C | 324 | 13.166 | 9.595 | 7.711 | 1.00 | 89.53 | C |
| ATOM | 9809 | NZ | LYS | C | 324 | 13.169 | 8.139 | 8.045 | 1.00 | 89.53 | C |
| ATOM | 9810 | C | LYS | C | 324 | 15.859 | 13.049 | 3.295 | 1.00 | 63.12 | C |
| ATOM | 9811 | O | LYS | C | 324 | 17.050 | 13.270 | 3.502 | 1.00 | 63.12 | C |
| ATOM | 9812 | N | MET | C | 325 | 15.388 | 12.767 | 2.091 | 1.00 | 75.86 | C |
| ATOM | 9813 | CA | MET | C | 325 | 16.281 | 12.693 | 0.948 | 1.00 | 75.86 | C |
| ATOM | 9814 | CB | MET | C | 325 | 15.587 | 11.967 | −0.201 | 1.00 | 91.27 | C |
| ATOM | 9815 | CG | MET | C | 325 | 15.281 | 10.527 | 0.113 | 1.00 | 79.83 | C |
| ATOM | 9816 | SD | MET | C | 325 | 14.464 | 9.691 | −1.238 | 1.00 | 79.83 | C |
| ATOM | 9817 | CE | MET | C | 325 | 12.784 | 9.830 | −0.746 | 1.00 | 79.83 | C |
| ATOM | 9818 | C | MET | C | 325 | 16.783 | 14.043 | 0.470 | 1.00 | 75.86 | C |
| ATOM | 9819 | O | MET | C | 325 | 17.732 | 14.117 | −0.308 | 1.00 | 75.86 | C |
| ATOM | 9820 | N | ALA | C | 326 | 16.156 | 15.113 | 0.932 | 1.00 | 58.65 | C |
| ATOM | 9821 | CA | ALA | C | 326 | 16.573 | 16.436 | 0.506 | 1.00 | 58.65 | C |
| ATOM | 9822 | CB | ALA | C | 326 | 15.350 | 17.348 | 0.362 | 1.00 | 60.09 | C |
| ATOM | 9823 | C | ALA | C | 326 | 17.580 | 17.050 | 1.470 | 1.00 | 58.65 | C |
| ATOM | 9824 | O | ALA | C | 326 | 18.402 | 17.866 | 1.067 | 1.00 | 58.65 | C |
| ATOM | 9825 | N | LYS | C | 327 | 17.529 | 16.630 | 2.729 | 1.00 | 129.75 | C |
| ATOM | 9826 | CA | LYS | C | 327 | 18.398 | 17.169 | 3.768 | 1.00 | 129.75 | C |
| ATOM | 9827 | CB | LYS | C | 327 | 18.803 | 16.086 | 4.763 | 1.00 | 157.86 | C |
| ATOM | 9828 | CG | LYS | C | 327 | 19.442 | 16.675 | 6.012 | 1.00 | 76.15 | C |
| ATOM | 9829 | CD | LYS | C | 327 | 19.723 | 15.618 | 7.062 | 1.00 | 76.15 | C |
| ATOM | 9830 | CE | LYS | C | 327 | 20.225 | 16.247 | 8.360 | 1.00 | 76.15 | C |
| ATOM | 9831 | NZ | LYS | C | 327 | 20.527 | 15.223 | 9.410 | 1.00 | 76.15 | C |
| ATOM | 9832 | C | LYS | C | 327 | 19.651 | 17.890 | 3.297 | 1.00 | 129.75 | C |
| ATOM | 9833 | O | LYS | C | 327 | 19.771 | 19.103 | 3.469 | 1.00 | 129.75 | C |
| ATOM | 9834 | N | GLY | C | 328 | 20.582 | 17.162 | 2.695 | 1.00 | 73.50 | C |
| ATOM | 9835 | CA | GLY | C | 328 | 21.804 | 17.816 | 2.261 | 1.00 | 73.50 | C |
| ATOM | 9836 | C | GLY | C | 328 | 21.846 | 18.449 | 0.881 | 1.00 | 73.50 | C |
| ATOM | 9837 | O | GLY | C | 328 | 22.632 | 19.362 | 0.656 | 1.00 | 73.50 | C |
| ATOM | 9838 | N | LYS | C | 329 | 21.001 | 17.999 | −0.039 | 1.00 | 68.45 | C |
| ATOM | 9839 | CA | LYS | C | 329 | 21.039 | 18.530 | −1.392 | 1.00 | 68.45 | C |
| ATOM | 9840 | CB | LYS | C | 329 | 20.729 | 17.403 | −2.375 | 1.00 | 198.57 | C |
| ATOM | 9841 | CG | LYS | C | 329 | 21.775 | 16.304 | −2.373 | 1.00 | 198.57 | C |
| ATOM | 9842 | CD | LYS | C | 329 | 21.671 | 15.474 | −3.634 | 1.00 | 198.57 | C |
| ATOM | 9843 | CE | LYS | C | 329 | 22.751 | 14.410 | −3.715 | 1.00 | 88.33 | C |
| ATOM | 9844 | NZ | LYS | C | 329 | 22.634 | 13.643 | −4.995 | 1.00 | 88.33 | C |
| ATOM | 9845 | C | LYS | C | 329 | 20.248 | 19.783 | −1.801 | 1.00 | 68.45 | C |
| ATOM | 9846 | O | LYS | C | 329 | 20.073 | 20.019 | −2.997 | 1.00 | 68.45 | C |
| ATOM | 9847 | N | ILE | C | 330 | 19.777 | 20.593 | −0.853 | 1.00 | 69.95 | C |
| ATOM | 9848 | CA | ILE | C | 330 | 19.045 | 21.811 | −1.224 | 1.00 | 69.95 | C |
| ATOM | 9849 | CB | ILE | C | 330 | 17.499 | 21.622 | −1.158 | 1.00 | 63.51 | C |
| ATOM | 9850 | CG2 | ILE | C | 330 | 17.074 | 20.439 | −2.020 | 1.00 | 64.43 | C |
| ATOM | 9851 | CG1 | ILE | C | 330 | 17.042 | 21.440 | 0.291 | 1.00 | 64.43 | C |
| ATOM | 9852 | CD1 | ILE | C | 330 | 15.551 | 21.623 | 0.474 | 1.00 | 64.43 | C |
| ATOM | 9853 | C | ILE | C | 330 | 19.437 | 22.975 | −0.315 | 1.00 | 69.95 | C |
| ATOM | 9854 | O | ILE | C | 330 | 20.120 | 22.775 | 0.692 | 1.00 | 69.95 | C |
| ATOM | 9855 | N | PRO | C | 331 | 19.011 | 24.207 | −0.656 | 1.00 | 63.43 | C |
| ATOM | 9856 | CD | PRO | C | 331 | 18.158 | 24.594 | −1.792 | 1.00 | 63.75 | C |
| ATOM | 9857 | CA | PRO | C | 331 | 19.341 | 25.382 | 0.156 | 1.00 | 63.43 | C |
| ATOM | 9858 | CB | PRO | C | 331 | 18.394 | 26.444 | −0.380 | 1.00 | 200.84 | C |
| ATOM | 9859 | CG | PRO | C | 331 | 18.314 | 26.101 | −1.802 | 1.00 | 63.75 | C |
| ATOM | 9860 | C | PRO | C | 331 | 19.128 | 25.127 | 1.635 | 1.00 | 63.43 | C |
| ATOM | 9861 | O | PRO | C | 331 | 18.123 | 24.533 | 2.025 | 1.00 | 63.43 | C |
| ATOM | 9862 | N | LYS | C | 332 | 20.077 | 25.565 | 2.454 | 1.00 | 52.99 | C |
| ATOM | 9863 | CA | LYS | C | 332 | 19.972 | 25.390 | 3.896 | 1.00 | 53.61 | C |
| ATOM | 9864 | CB | LYS | C | 332 | 21.312 | 25.693 | 4.555 | 1.00 | 122.66 | C |
| ATOM | 9865 | CG | LYS | C | 332 | 21.772 | 27.113 | 4.314 | 1.00 | 94.20 | C |
| ATOM | 9866 | CD | LYS | C | 332 | 22.808 | 27.537 | 5.329 | 1.00 | 94.20 | C |
| ATOM | 9867 | CE | LYS | C | 332 | 23.094 | 29.024 | 5.221 | 1.00 | 94.20 | C |
| ATOM | 9868 | NZ | LYS | C | 332 | 24.014 | 29.486 | 6.300 | 1.00 | 94.20 | C |
| ATOM | 9869 | C | LYS | C | 332 | 18.905 | 26.343 | 4.458 | 1.00 | 52.68 | C |
| ATOM | 9870 | O | LYS | C | 332 | 18.573 | 27.359 | 3.840 | 1.00 | 50.77 | C |
| ATOM | 9871 | N | ASP | C | 333 | 18.368 | 26.009 | 5.627 | 1.00 | 60.44 | C |
| ATOM | 9872 | CA | ASP | C | 333 | 17.361 | 26.846 | 6.264 | 1.00 | 59.90 | C |
| ATOM | 9873 | CB | ASP | C | 333 | 16.857 | 26.186 | 7.544 | 1.00 | 136.89 | C |
| ATOM | 9874 | CG | ASP | C | 333 | 15.673 | 25.282 | 7.302 | 1.00 | 104.48 | C |
| ATOM | 9875 | OD1 | ASP | C | 333 | 15.641 | 24.608 | 6.255 | 1.00 | 104.48 | C |
| ATOM | 9876 | OD2 | ASP | C | 333 | 14.775 | 25.240 | 8.165 | 1.00 | 104.48 | C |
| ATOM | 9877 | C | ASP | C | 333 | 17.982 | 28.185 | 6.593 | 1.00 | 63.75 | C |
| ATOM | 9878 | O | ASP | C | 333 | 19.144 | 28.259 | 6.983 | 1.00 | 63.40 | C |
| ATOM | 9879 | N | THR | C | 334 | 17.209 | 29.249 | 6.430 | 1.00 | 69.18 | C |
| ATOM | 9880 | CA | THR | C | 334 | 17.705 | 30.589 | 6.717 | 1.00 | 68.90 | C |
| ATOM | 9881 | CB | THR | C | 334 | 18.118 | 31.322 | 5.415 | 1.00 | 98.22 | C |
| ATOM | 9882 | OG1 | THR | C | 334 | 19.342 | 30.762 | 4.918 | 1.00 | 84.12 | C |
| ATOM | 9883 | CG2 | THR | C | 334 | 18.306 | 32.814 | 5.667 | 1.00 | 84.12 | C |
| ATOM | 9884 | C | THR | C | 334 | 16.645 | 31.392 | 7.441 | 1.00 | 75.34 | C |
| ATOM | 9885 | O | THR | C | 334 | 15.543 | 31.591 | 6.930 | 1.00 | 72.70 | C |
| ATOM | 9886 | N | THR | C | 335 | 16.992 | 31.849 | 8.638 | 1.00 | 52.32 | C |
| ATOM | 9887 | CA | THR | C | 335 | 16.076 | 32.632 | 9.458 | 1.00 | 52.11 | C |
| ATOM | 9888 | CB | THR | C | 335 | 16.523 | 32.648 | 10.898 | 1.00 | 59.42 | C |
| ATOM | 9889 | OG1 | THR | C | 335 | 16.858 | 31.315 | 11.302 | 1.00 | 48.78 | C |
| ATOM | 9890 | CG2 | THR | C | 335 | 15.406 | 33.168 | 11.769 | 1.00 | 48.78 | C |
| ATOM | 9891 | C | THR | C | 335 | 15.934 | 34.072 | 8.992 | 1.00 | 52.97 | C |
| ATOM | 9892 | O | THR | C | 335 | 16.918 | 34.768 | 8.771 | 1.00 | 54.32 | C |
| ATOM | 9893 | N | PRO | C | 336 | 14.695 | 34.548 | 8.871 | 1.00 | 49.92 | C |
| ATOM | 9894 | CD | PRO | C | 336 | 13.422 | 33.860 | 9.149 | 1.00 | 45.64 | C |
| ATOM | 9895 | CA | PRO | C | 336 | 14.446 | 35.909 | 8.418 | 1.00 | 49.50 | C |
| ATOM | 9896 | CB | PRO | C | 336 | 13.019 | 35.820 | 7.907 | 1.00 | 54.50 | C |
| ATOM | 9897 | CG | PRO | C | 336 | 12.397 | 34.974 | 8.971 | 1.00 | 45.64 | C |
| ATOM | 9898 | C | PRO | C | 336 | 14.585 | 36.968 | 9.493 | 1.00 | 49.54 | C |
| ATOM | 9899 | O | PRO | C | 336 | 14.781 | 36.662 | 10.666 | 1.00 | 50.17 | C |
| ATOM | 9900 | N | THR | C | 337 | 14.467 | 38.215 | 9.048 | 1.00 | 65.01 | C |
| ATOM | 9901 | CA | THR | C | 337 | 14.527 | 39.400 | 9.886 | 1.00 | 68.47 | C |
| ATOM | 9902 | CB | THR | C | 337 | 15.537 | 40.405 | 9.333 | 1.00 | 114.14 | C |
| ATOM | 9903 | OG1 | THR | C | 337 | 16.860 | 39.915 | 9.564 | 1.00 | 65.03 | C |
| ATOM | 9904 | CG2 | THR | C | 337 | 15.365 | 41.755 | 9.989 | 1.00 | 65.03 | C |
| ATOM | 9905 | C | THR | C | 337 | 13.137 | 39.987 | 9.761 | 1.00 | 76.04 | C |
| ATOM | 9906 | O | THR | C | 337 | 12.628 | 40.134 | 8.654 | 1.00 | 67.42 | C |
| ATOM | 9907 | N | VAL | C | 338 | 12.513 | 40.310 | 10.882 | 1.00 | 47.45 | C |
| ATOM | 9908 | CA | VAL | C | 338 | 11.170 | 40.874 | 10.824 | 1.00 | 54.31 | C |
| ATOM | 9909 | CB | VAL | C | 338 | 10.147 | 39.969 | 11.542 | 1.00 | 42.27 | C |
| ATOM | 9910 | CG1 | VAL | C | 338 | 8.731 | 40.444 | 11.249 | 1.00 | 41.89 | C |
| ATOM | 9911 | CG2 | VAL | C | 338 | 10.330 | 38.535 | 11.094 | 1.00 | 41.89 | C |
| ATOM | 9912 | C | VAL | C | 338 | 11.098 | 42.264 | 11.430 | 1.00 | 58.78 | C |
| ATOM | 9913 | O | VAL | C | 338 | 11.814 | 42.576 | 12.379 | 1.00 | 58.61 | C |
| ATOM | 9914 | N | LYS | C | 339 | 10.225 | 43.090 | 10.859 | 1.00 | 59.26 | C |
| ATOM | 9915 | CA | LYS | C | 339 | 10.012 | 44.459 | 11.307 | 1.00 | 57.14 | C |
| ATOM | 9916 | CB | LYS | C | 339 | 11.005 | 45.400 | 10.617 | 1.00 | 161.77 | C |
| ATOM | 9917 | CG | LYS | C | 339 | 12.454 | 45.136 | 10.992 | 1.00 | 130.08 | C |
| ATOM | 9918 | CD | LYS | C | 339 | 13.414 | 45.447 | 9.849 | 1.00 | 130.08 | C |
| ATOM | 9919 | CE | LYS | C | 339 | 14.837 | 45.019 | 10.200 | 1.00 | 130.08 | C |
| ATOM | 9920 | NZ | LYS | C | 339 | 15.760 | 45.103 | 9.035 | 1.00 | 130.08 | C |
| ATOM | 9921 | C | LYS | C | 339 | 8.596 | 44.806 | 10.901 | 1.00 | 66.13 | C |
| ATOM | 9922 | O | LYS | C | 339 | 8.185 | 44.529 | 9.778 | 1.00 | 58.79 | C |
| ATOM | 9923 | N | VAL | C | 340 | 7.836 | 45.380 | 11.819 | 1.00 | 65.63 | C |
| ATOM | 9924 | CA | VAL | C | 340 | 6.464 | 45.775 | 11.517 | 1.00 | 63.06 | C |
| ATOM | 9925 | CB | VAL | C | 340 | 5.492 | 45.310 | 12.604 | 1.00 | 69.41 | C |
| ATOM | 9926 | CG1 | VAL | C | 340 | 4.092 | 45.783 | 12.275 | 1.00 | 54.87 | C |
| ATOM | 9927 | CG2 | VAL | C | 340 | 5.545 | 43.801 | 12.732 | 1.00 | 54.87 | C |
| ATOM | 9928 | C | VAL | C | 340 | 6.419 | 47.295 | 11.470 | 1.00 | 65.63 | C |
| ATOM | 9929 | O | VAL | C | 340 | 6.799 | 47.938 | 12.431 | 1.00 | 62.48 | C |
| ATOM | 9930 | N | THR | C | 341 | 5.955 | 47.875 | 10.369 | 1.00 | 91.39 | C |
| ATOM | 9931 | CA | THR | C | 341 | 5.897 | 49.328 | 10.269 | 1.00 | 87.29 | C |
| ATOM | 9932 | CB | THR | C | 341 | 6.600 | 49.820 | 9.003 | 1.00 | 98.94 | C |
| ATOM | 9933 | OG1 | THR | C | 341 | 7.970 | 49.408 | 9.038 | 1.00 | 80.30 | C |
| ATOM | 9934 | CG2 | THR | C | 341 | 6.530 | 51.338 | 8.909 | 1.00 | 80.30 | C |
| ATOM | 9935 | C | THR | C | 341 | 4.472 | 49.856 | 10.270 | 1.00 | 89.44 | C |
| ATOM | 9936 | O | THR | C | 341 | 3.560 | 49.225 | 9.734 | 1.00 | 91.04 | C |
| ATOM | 9937 | N | GLY | C | 342 | 4.289 | 51.023 | 10.875 | 1.00 | 95.13 | C |
| ATOM | 9938 | CA | GLY | C | 342 | 2.971 | 51.615 | 10.935 | 1.00 | 99.30 | C |
| ATOM | 9939 | C | GLY | C | 342 | 2.836 | 52.836 | 10.051 | 1.00 | 98.27 | C |
| ATOM | 9940 | O | GLY | C | 342 | 2.638 | 53.940 | 10.548 | 1.00 | 92.54 | C |
| ATOM | 9941 | N | ASN | C | 343 | 2.950 | 52.646 | 8.741 | 1.00 | 100.57 | C |
| ATOM | 9942 | CA | ASN | C | 343 | 2.810 | 53.752 | 7.796 | 1.00 | 106.98 | C |
| ATOM | 9943 | CB | ASN | C | 343 | 3.170 | 53.294 | 6.382 | 1.00 | 174.08 | C |
| ATOM | 9944 | CG | ASN | C | 343 | 4.523 | 52.627 | 6.312 | 1.00 | 153.88 | C |
| ATOM | 9945 | OD1 | ASN | C | 343 | 5.549 | 53.244 | 6.605 | 1.00 | 153.88 | C |
| ATOM | 9946 | ND2 | ASN | C | 343 | 4.536 | 51.357 | 5.924 | 1.00 | 153.88 | C |
| ATOM | 9947 | C | ASN | C | 343 | 1.355 | 54.204 | 7.810 | 1.00 | 105.04 | C |
| ATOM | 9948 | O | ASN | C | 343 | 0.596 | 53.857 | 8.712 | 1.00 | 93.67 | C |
| ATOM | 9949 | N | GLU | C | 344 | 0.967 | 54.976 | 6.806 | 1.00 | 160.05 | C |
| ATOM | 9950 | CA | GLU | C | 344 | −0.408 | 55.434 | 6.708 | 1.00 | 160.05 | C |
| ATOM | 9951 | CB | GLU | C | 344 | −0.673 | 56.509 | 7.761 | 1.00 | 200.84 | C |
| ATOM | 9952 | CG | GLU | C | 344 | −1.166 | 55.957 | 9.097 | 1.00 | 190.23 | C |
| ATOM | 9953 | CD | GLU | C | 344 | −1.279 | 56.994 | 10.205 | 1.00 | 190.23 | C |
| ATOM | 9954 | OE1 | GLU | C | 344 | −0.239 | 57.393 | 10.758 | 1.00 | 190.23 | C |
| ATOM | 9955 | OE2 | GLU | C | 344 | −2.411 | 57.407 | 10.535 | 1.00 | 190.23 | C |
| ATOM | 9956 | C | GLU | C | 344 | −0.729 | 55.921 | 5.294 | 1.00 | 160.05 | C |
| ATOM | 9957 | O | GLU | C | 344 | −0.264 | 56.966 | 4.850 | 1.00 | 160.05 | C |
| ATOM | 9958 | N | LEU | C | 345 | −1.553 | 55.136 | 4.602 | 1.00 | 200.84 | C |
| ATOM | 9959 | CA | LEU | C | 345 | −1.951 | 55.402 | 3.221 | 1.00 | 200.84 | C |
| ATOM | 9960 | CB | LEU | C | 345 | −2.536 | 54.116 | 2.613 | 1.00 | 200.84 | C |
| ATOM | 9961 | CG | LEU | C | 345 | −1.624 | 52.879 | 2.554 | 1.00 | 166.21 | C |
| ATOM | 9962 | CD1 | LEU | C | 345 | −2.353 | 51.728 | 1.866 | 1.00 | 166.21 | C |
| ATOM | 9963 | CD2 | LEU | C | 345 | −0.343 | 53.213 | 1.799 | 1.00 | 166.21 | C |
| ATOM | 9964 | C | LEU | C | 345 | −2.897 | 56.587 | 2.940 | 1.00 | 200.84 | C |
| ATOM | 9965 | O | LEU | C | 345 | −3.731 | 56.521 | 2.036 | 1.00 | 200.84 | C |
| ATOM | 9966 | N | GLU | C | 346 | −2.759 | 57.665 | 3.707 | 1.00 | 154.12 | C |
| ATOM | 9967 | CA | GLU | C | 346 | −3.551 | 58.890 | 3.536 | 1.00 | 154.12 | C |
| ATOM | 9968 | CB | GLU | C | 346 | −3.228 | 59.488 | 2.174 | 1.00 | 167.08 | C |
| ATOM | 9969 | CG | GLU | C | 346 | −1.742 | 59.423 | 1.874 | 1.00 | 152.51 | C |
| ATOM | 9970 | CD | GLU | C | 346 | −1.311 | 60.390 | 0.802 | 1.00 | 152.51 | C |
| ATOM | 9971 | OE1 | GLU | C | 346 | −1.386 | 61.611 | 1.048 | 1.00 | 152.51 | C |
| ATOM | 9972 | OE2 | GLU | C | 346 | −0.898 | 59.931 | −0.283 | 1.00 | 152.51 | C |
| ATOM | 9973 | C | GLU | C | 346 | −5.075 | 58.878 | 3.750 | 1.00 | 154.12 | C |
| ATOM | 9974 | O | GLU | C | 346 | −5.567 | 59.405 | 4.751 | 1.00 | 154.12 | C |
| ATOM | 9975 | N | ASP | C | 347 | −5.827 | 58.304 | 2.817 | 1.00 | 176.33 | C |
| ATOM | 9976 | CA | ASP | C | 347 | −7.284 | 58.278 | 2.952 | 1.00 | 176.33 | C |
| ATOM | 9977 | CB | ASP | C | 347 | −7.933 | 57.686 | 1.693 | 1.00 | 169.88 | C |
| ATOM | 9978 | CG | ASP | C | 347 | −7.960 | 56.169 | 1.702 | 1.00 | 169.88 | C |
| ATOM | 9979 | OD1 | ASP | C | 347 | −6.876 | 55.552 | 1.726 | 1.00 | 169.88 | C |
| ATOM | 9980 | OD2 | ASP | C | 347 | −9.071 | 55.595 | 1.685 | 1.00 | 169.88 | C |
| ATOM | 9981 | C | ASP | C | 347 | −7.763 | 57.511 | 4.189 | 1.00 | 176.33 | C |
| ATOM | 9982 | O | ASP | C | 347 | −8.735 | 57.907 | 4.835 | 1.00 | 176.33 | C |
| ATOM | 9983 | N | GLY | C | 348 | −7.083 | 56.416 | 4.516 | 1.00 | 156.15 | C |
| ATOM | 9984 | CA | GLY | C | 348 | −7.463 | 55.625 | 5.677 | 1.00 | 153.46 | C |
| ATOM | 9985 | C | GLY | C | 348 | −6.629 | 55.973 | 6.894 | 1.00 | 150.42 | C |
| ATOM | 9986 | O | GLY | C | 348 | −5.509 | 56.370 | 6.767 | 1.00 | 145.43 | C |
| ATOM | 9987 | N | ASN | C | 349 | −7.170 | 55.831 | 8.091 | 1.00 | 200.84 | C |
| ATOM | 9988 | CA | ASN | C | 349 | −6.402 | 56.167 | 9.285 | 1.00 | 200.84 | C |
| ATOM | 9989 | CB | ASN | C | 349 | −7.200 | 55.785 | 10.534 | 1.00 | 183.75 | C |
| ATOM | 9990 | CG | ASN | C | 349 | −8.020 | 54.525 | 10.339 | 1.00 | 168.93 | C |
| ATOM | 9991 | OD1 | ASN | C | 349 | −8.910 | 54.470 | 9.488 | 1.00 | 168.93 | C |
| ATOM | 9992 | ND2 | ASN | C | 349 | −7.726 | 53.506 | 11.131 | 1.00 | 168.93 | C |
| ATOM | 9993 | C | ASN | C | 349 | −4.989 | 55.565 | 9.342 | 1.00 | 200.84 | C |
| ATOM | 9994 | O | ASN | C | 349 | −4.021 | 56.236 | 8.989 | 1.00 | 200.84 | C |
| ATOM | 9995 | N | MET | C | 350 | −4.865 | 54.310 | 9.766 | 1.00 | 116.77 | C |
| ATOM | 9996 | CA | MET | C | 350 | −3.549 | 53.680 | 9.877 | 1.00 | 112.86 | C |
| ATOM | 9997 | CB | MET | C | 350 | −3.260 | 53.380 | 11.354 | 1.00 | 187.20 | C |
| ATOM | 9998 | CG | MET | C | 350 | −1.925 | 52.708 | 11.621 | 1.00 | 141.88 | C |
| ATOM | 9999 | SD | MET | C | 350 | −1.602 | 52.511 | 13.380 | 1.00 | 141.88 | C |
| ATOM | 10000 | CE | MET | C | 350 | −0.435 | 53.857 | 13.651 | 1.00 | 141.88 | C |
| ATOM | 10001 | C | MET | C | 350 | −3.360 | 52.406 | 9.045 | 1.00 | 110.00 | C |
| ATOM | 10002 | O | MET | C | 350 | −4.325 | 51.733 | 8.687 | 1.00 | 106.43 | C |
| ATOM | 10003 | N | THR | C | 351 | −2.102 | 52.090 | 8.741 | 1.00 | 98.32 | C |
| ATOM | 10004 | CA | THR | C | 351 | −1.758 | 50.895 | 7.971 | 1.00 | 96.28 | C |
| ATOM | 10005 | CB | THR | C | 351 | −1.421 | 51.246 | 6.496 | 1.00 | 93.91 | C |
| ATOM | 10006 | OG1 | THR | C | 351 | −1.818 | 50.166 | 5.653 | 1.00 | 61.03 | C |
| ATOM | 10007 | CG2 | THR | C | 351 | 0.078 | 51.446 | 6.298 | 1.00 | 61.03 | C |
| ATOM | 10008 | C | THR | C | 351 | −0.549 | 50.187 | 8.606 | 1.00 | 90.51 | C |
| ATOM | 10009 | O | THR | C | 351 | 0.431 | 50.827 | 8.986 | 1.00 | 82.10 | C |
| ATOM | 10010 | N | LEU | C | 352 | −0.624 | 48.866 | 8.735 | 1.00 | 50.29 | C |
| ATOM | 10011 | CA | LEU | C | 352 | 0.476 | 48.097 | 9.311 | 1.00 | 53.31 | C |
| ATOM | 10012 | CB | LEU | C | 352 | −0.027 | 47.231 | 10.458 | 1.00 | 92.49 | C |
| ATOM | 10013 | CG | LEU | C | 352 | −0.196 | 48.045 | 11.740 | 1.00 | 73.55 | C |
| ATOM | 10014 | CD1 | LEU | C | 352 | −0.959 | 47.257 | 12.789 | 1.00 | 73.55 | C |
| ATOM | 10015 | CD2 | LEU | C | 352 | 1.177 | 48.433 | 12.243 | 1.00 | 73.55 | C |
| ATOM | 10016 | C | LEU | C | 352 | 1.132 | 47.241 | 8.251 | 1.00 | 49.99 | C |
| ATOM | 10017 | O | LEU | C | 352 | 0.461 | 46.620 | 7.427 | 1.00 | 45.61 | C |
| ATOM | 10018 | N | GLU | C | 353 | 2.455 | 47.224 | 8.265 | 1.00 | 77.85 | C |
| ATOM | 10019 | CA | GLU | C | 353 | 3.206 | 46.460 | 7.285 | 1.00 | 74.10 | C |
| ATOM | 10020 | CB | GLU | C | 353 | 3.907 | 47.408 | 6.326 | 1.00 | 142.84 | C |
| ATOM | 10021 | CG | GLU | C | 353 | 4.702 | 46.712 | 5.256 | 1.00 | 101.39 | C |
| ATOM | 10022 | CD | GLU | C | 353 | 5.449 | 47.692 | 4.380 | 1.00 | 101.39 | C |
| ATOM | 10023 | OE1 | GLU | C | 353 | 4.796 | 48.585 | 3.802 | 1.00 | 101.39 | C |
| ATOM | 10024 | OE2 | GLU | C | 353 | 6.688 | 47.573 | 4.270 | 1.00 | 101.39 | C |
| ATOM | 10025 | C | GLU | C | 353 | 4.232 | 45.562 | 7.956 | 1.00 | 77.35 | C |
| ATOM | 10026 | O | GLU | C | 353 | 5.166 | 46.036 | 8.595 | 1.00 | 78.13 | C |
| ATOM | 10027 | N | CYS | C | 354 | 4.040 | 44.259 | 7.807 | 1.00 | 71.50 | C |
| ATOM | 10028 | CA | CYS | C | 354 | 4.932 | 43.264 | 8.378 | 1.00 | 71.78 | C |
| ATOM | 10029 | C | CYS | C | 354 | 5.857 | 42.832 | 7.254 | 1.00 | 72.87 | C |
| ATOM | 10030 | O | CYS | C | 354 | 5.393 | 42.373 | 6.213 | 1.00 | 71.57 | C |
| ATOM | 10031 | CB | CYS | C | 354 | 4.117 | 42.064 | 8.853 | 1.00 | 121.98 | C |
| ATOM | 10032 | SG | CYS | C | 354 | 4.968 | 40.890 | 9.953 | 1.00 | 99.17 | C |
| ATOM | 10033 | N | THR | C | 355 | 7.160 | 42.992 | 7.436 | 1.00 | 60.18 | C |
| ATOM | 10034 | CA | THR | C | 355 | 8.077 | 42.579 | 6.384 | 1.00 | 49.54 | C |
| ATOM | 10035 | CB | THR | C | 355 | 8.852 | 43.768 | 5.769 | 1.00 | 128.19 | C |
| ATOM | 10036 | OG1 | THR | C | 355 | 10.172 | 43.817 | 6.314 | 1.00 | 77.34 | C |
| ATOM | 10037 | CG2 | THR | C | 355 | 8.145 | 45.076 | 6.054 | 1.00 | 77.34 | C |
| ATOM | 10038 | C | THR | C | 355 | 9.068 | 41.569 | 6.938 | 1.00 | 63.03 | C |
| ATOM | 10039 | O | THR | C | 355 | 9.711 | 41.798 | 7.966 | 1.00 | 55.83 | C |
| ATOM | 10040 | N | VAL | C | 356 | 9.168 | 40.444 | 6.242 | 1.00 | 47.18 | C |
| ATOM | 10041 | CA | VAL | C | 356 | 10.044 | 39.353 | 6.620 | 1.00 | 42.90 | C |
| ATOM | 10042 | CB | VAL | C | 356 | 9.251 | 38.052 | 6.649 | 1.00 | 62.19 | C |
| ATOM | 10043 | CG1 | VAL | C | 356 | 10.083 | 36.944 | 7.253 | 1.00 | 46.72 | C |
| ATOM | 10044 | CG2 | VAL | C | 356 | 7.980 | 38.272 | 7.438 | 1.00 | 46.72 | C |
| ATOM | 10045 | C | VAL | C | 356 | 11.154 | 39.290 | 5.578 | 1.00 | 51.74 | C |
| ATOM | 10046 | O | VAL | C | 356 | 10.901 | 39.084 | 4.392 | 1.00 | 52.85 | C |
| ATOM | 10047 | N | ASN | C | 357 | 12.388 | 39.463 | 6.037 | 1.00 | 51.17 | C |
| ATOM | 10048 | CA | ASN | C | 357 | 13.537 | 39.490 | 5.143 | 1.00 | 47.39 | C |
| ATOM | 10049 | CB | ASN | C | 357 | 14.412 | 40.689 | 5.479 | 1.00 | 118.85 | C |
| ATOM | 10050 | CG | ASN | C | 357 | 13.696 | 41.985 | 5.254 | 1.00 | 72.77 | C |
| ATOM | 10051 | OD1 | ASN | C | 357 | 13.166 | 42.226 | 4.171 | 1.00 | 72.77 | C |
| ATOM | 10052 | ND2 | ASN | C | 357 | 13.663 | 42.831 | 6.275 | 1.00 | 72.77 | C |
| ATOM | 10053 | C | ASN | C | 357 | 14.407 | 38.264 | 5.036 | 1.00 | 51.55 | C |
| ATOM | 10054 | O | ASN | C | 357 | 14.893 | 37.720 | 6.032 | 1.00 | 54.43 | C |
| ATOM | 10055 | N | SER | C | 358 | 14.618 | 37.876 | 3.785 | 1.00 | 48.88 | C |
| ATOM | 10056 | CA | SER | C | 358 | 15.418 | 36.725 | 3.392 | 1.00 | 56.18 | C |
| ATOM | 10057 | CB | SER | C | 358 | 16.863 | 37.163 | 3.117 | 1.00 | 69.78 | C |
| ATOM | 10058 | OG | SER | C | 358 | 17.497 | 37.597 | 4.296 | 1.00 | 68.12 | C |
| ATOM | 10059 | C | SER | C | 358 | 15.390 | 35.505 | 4.325 | 1.00 | 60.90 | C |
| ATOM | 10060 | O | SER | C | 358 | 16.210 | 35.369 | 5.237 | 1.00 | 56.45 | C |
| ATOM | 10061 | N | PHE | C | 359 | 14.433 | 34.618 | 4.055 | 1.00 | 55.44 | C |
| ATOM | 10062 | CA | PHE | C | 359 | 14.225 | 33.373 | 4.795 | 1.00 | 49.75 | C |
| ATOM | 10063 | CB | PHE | C | 359 | 12.829 | 33.390 | 5.422 | 1.00 | 50.82 | C |
| ATOM | 10064 | CG | PHE | C | 359 | 11.736 | 33.843 | 4.483 | 1.00 | 45.42 | C |
| ATOM | 10065 | CD1 | PHE | C | 359 | 11.150 | 32.956 | 3.577 | 1.00 | 45.42 | C |
| ATOM | 10066 | CD2 | PHE | C | 359 | 11.276 | 35.161 | 4.518 | 1.00 | 45.42 | C |
| ATOM | 10067 | CE1 | PHE | C | 359 | 10.120 | 33.372 | 2.718 | 1.00 | 45.42 | C |
| ATOM | 10068 | CE2 | PHE | C | 359 | 10.245 | 35.590 | 3.665 | 1.00 | 45.42 | C |
| ATOM | 10069 | CZ | PHE | C | 359 | 9.667 | 34.690 | 2.764 | 1.00 | 45.42 | C |
| ATOM | 10070 | C | PHE | C | 359 | 14.335 | 32.253 | 3.765 | 1.00 | 56.34 | C |
| ATOM | 10071 | O | PHE | C | 359 | 13.946 | 32.474 | 2.619 | 1.00 | 53.89 | C |
| ATOM | 10072 | N | TYR | C | 360 | 14.850 | 31.067 | 4.108 | 1.00 | 127.62 | C |
| ATOM | 10073 | CA | TYR | C | 360 | 14.918 | 30.086 | 3.032 | 1.00 | 127.40 | C |
| ATOM | 10074 | CB | TYR | C | 360 | 16.015 | 29.026 | 3.157 | 1.00 | 186.99 | C |
| ATOM | 10075 | CG | TYR | C | 360 | 15.769 | 27.984 | 2.058 | 1.00 | 74.13 | C |
| ATOM | 10076 | CD1 | TYR | C | 360 | 15.489 | 28.391 | 0.742 | 1.00 | 74.13 | C |
| ATOM | 10077 | CE1 | TYR | C | 360 | 15.077 | 27.482 | −0.238 | 1.00 | 74.13 | C |
| ATOM | 10078 | CD2 | TYR | C | 360 | 15.646 | 26.625 | 2.353 | 1.00 | 74.13 | C |
| ATOM | 10079 | CE2 | TYR | C | 360 | 15.240 | 25.700 | 1.368 | 1.00 | 74.13 | C |
| ATOM | 10080 | CZ | TYR | C | 360 | 14.953 | 26.137 | 0.080 | 1.00 | 74.13 | C |
| ATOM | 10081 | OH | TYR | C | 360 | 14.540 | 25.237 | −0.883 | 1.00 | 74.13 | C |
| ATOM | 10082 | C | TYR | C | 360 | 13.671 | 29.330 | 2.659 | 1.00 | 128.49 | C |
| ATOM | 10083 | O | TYR | C | 360 | 13.022 | 29.691 | 1.686 | 1.00 | 134.42 | C |
| ATOM | 10084 | N | PRO | C | 361 | 13.334 | 28.254 | 3.390 | 1.00 | 57.94 | C |
| ATOM | 10085 | CD | PRO | C | 361 | 13.630 | 27.927 | 4.798 | 1.00 | 38.70 | C |
| ATOM | 10086 | CA | PRO | C | 361 | 12.108 | 27.596 | 2.907 | 1.00 | 53.53 | C |
| ATOM | 10087 | CB | PRO | C | 361 | 11.729 | 26.650 | 4.051 | 1.00 | 37.94 | C |
| ATOM | 10088 | CG | PRO | C | 361 | 12.323 | 27.319 | 5.269 | 1.00 | 38.70 | C |
| ATOM | 10089 | C | PRO | C | 361 | 11.127 | 28.726 | 2.607 | 1.00 | 60.93 | C |
| ATOM | 10090 | O | PRO | C | 361 | 10.698 | 29.455 | 3.495 | 1.00 | 58.10 | C |
| ATOM | 10091 | N | PRO | C | 362 | 10.821 | 28.921 | 1.315 | 1.00 | 63.79 | C |
| ATOM | 10092 | CD | PRO | C | 362 | 11.231 | 27.988 | 0.249 | 1.00 | 39.87 | C |
| ATOM | 10093 | CA | PRO | C | 362 | 9.923 | 29.963 | 0.793 | 1.00 | 57.50 | C |
| ATOM | 10094 | CB | PRO | C | 362 | 9.956 | 29.733 | −0.718 | 1.00 | 39.87 | C |
| ATOM | 10095 | CG | PRO | C | 362 | 10.212 | 28.264 | −0.819 | 1.00 | 39.87 | C |
| ATOM | 10096 | C | PRO | C | 362 | 8.512 | 30.064 | 1.339 | 1.00 | 60.10 | C |
| ATOM | 10097 | O | PRO | C | 362 | 7.844 | 31.080 | 1.141 | 1.00 | 64.86 | C |
| ATOM | 10098 | N | ASP | C | 363 | 8.054 | 29.024 | 2.025 | 1.00 | 47.72 | C |
| ATOM | 10099 | CA | ASP | C | 363 | 6.714 | 29.048 | 2.587 | 1.00 | 47.57 | C |
| ATOM | 10100 | CB | ASP | C | 363 | 6.184 | 27.618 | 2.845 | 1.00 | 83.50 | C |
| ATOM | 10101 | CG | ASP | C | 363 | 7.195 | 26.710 | 3.545 | 1.00 | 77.71 | C |
| ATOM | 10102 | OD1 | ASP | C | 363 | 8.308 | 26.512 | 2.999 | 1.00 | 77.71 | C |
| ATOM | 10103 | OD2 | ASP | C | 363 | 6.862 | 26.180 | 4.633 | 1.00 | 77.71 | C |
| ATOM | 10104 | C | ASP | C | 363 | 6.720 | 29.855 | 3.866 | 1.00 | 51.11 | C |
| ATOM | 10105 | O | ASP | C | 363 | 7.504 | 29.595 | 4.770 | 1.00 | 47.61 | C |
| ATOM | 10106 | N | VAL | C | 364 | 5.868 | 30.869 | 3.912 | 1.00 | 46.77 | C |
| ATOM | 10107 | CA | VAL | C | 364 | 5.739 | 31.714 | 5.088 | 1.00 | 49.09 | C |
| ATOM | 10108 | CB | VAL | C | 364 | 6.566 | 33.016 | 4.976 | 1.00 | 58.92 | C |
| ATOM | 10109 | CG1 | VAL | C | 364 | 6.052 | 34.038 | 5.947 | 1.00 | 37.31 | C |
| ATOM | 10110 | CG2 | VAL | C | 364 | 7.999 | 32.744 | 5.318 | 1.00 | 37.31 | C |
| ATOM | 10111 | C | VAL | C | 364 | 4.280 | 32.087 | 5.227 | 1.00 | 56.43 | C |
| ATOM | 10112 | O | VAL | C | 364 | 3.590 | 32.334 | 4.241 | 1.00 | 48.17 | C |
| ATOM | 10113 | N | ILE | C | 365 | 3.801 | 32.103 | 6.457 | 1.00 | 59.16 | C |
| ATOM | 10114 | CA | ILE | C | 365 | 2.431 | 32.487 | 6.689 | 1.00 | 53.21 | C |
| ATOM | 10115 | CB | ILE | C | 365 | 1.554 | 31.285 | 7.049 | 1.00 | 80.41 | C |
| ATOM | 10116 | CG2 | ILE | C | 365 | 0.169 | 31.761 | 7.454 | 1.00 | 56.85 | C |
| ATOM | 10117 | CG1 | ILE | C | 365 | 1.460 | 30.359 | 5.834 | 1.00 | 56.85 | C |
| ATOM | 10118 | CD1 | ILE | C | 365 | 0.571 | 29.146 | 6.023 | 1.00 | 56.85 | C |
| ATOM | 10119 | C | ILE | C | 365 | 2.419 | 33.522 | 7.789 | 1.00 | 59.97 | C |
| ATOM | 10120 | O | ILE | C | 365 | 3.234 | 33.494 | 8.711 | 1.00 | 60.74 | C |
| ATOM | 10121 | N | THR | C | 366 | 1.477 | 34.440 | 7.673 | 1.00 | 38.14 | C |
| ATOM | 10122 | CA | THR | C | 366 | 1.363 | 35.532 | 8.596 | 1.00 | 46.42 | C |
| ATOM | 10123 | CB | THR | C | 366 | 1.749 | 36.823 | 7.858 | 1.00 | 72.48 | C |
| ATOM | 10124 | OG1 | THR | C | 366 | 2.831 | 37.444 | 8.543 | 1.00 | 60.16 | C |
| ATOM | 10125 | CG2 | THR | C | 366 | 0.578 | 37.782 | 7.762 | 1.00 | 60.16 | C |
| ATOM | 10126 | C | THR | C | 366 | −0.034 | 35.651 | 9.170 | 1.00 | 55.40 | C |
| ATOM | 10127 | O | THR | C | 366 | −1.024 | 35.530 | 8.449 | 1.00 | 54.64 | C |
| ATOM | 10128 | N | LYS | C | 367 | −0.103 | 35.887 | 10.475 | 1.00 | 44.19 | C |
| ATOM | 10129 | CA | LYS | C | 367 | −1.374 | 36.062 | 11.168 | 1.00 | 47.39 | C |
| ATOM | 10130 | CB | LYS | C | 367 | −1.626 | 34.922 | 12.153 | 1.00 | 71.49 | C |
| ATOM | 10131 | CG | LYS | C | 367 | −1.972 | 33.616 | 11.496 | 1.00 | 62.50 | C |
| ATOM | 10132 | CD | LYS | C | 367 | −2.511 | 32.607 | 12.499 | 1.00 | 62.50 | C |
| ATOM | 10133 | CE | LYS | C | 367 | −3.048 | 31.385 | 11.773 | 1.00 | 62.50 | C |
| ATOM | 10134 | NZ | LYS | C | 367 | −3.843 | 30.509 | 12.670 | 1.00 | 62.50 | C |
| ATOM | 10135 | C | LYS | C | 367 | −1.383 | 37.374 | 11.932 | 1.00 | 50.09 | C |
| ATOM | 10136 | O | LYS | C | 367 | −0.447 | 37.669 | 12.679 | 1.00 | 50.40 | C |
| ATOM | 10137 | N | TRP | C | 368 | −2.439 | 38.159 | 11.744 | 1.00 | 48.65 | C |
| ATOM | 10138 | CA | TRP | C | 368 | −2.577 | 39.437 | 12.434 | 1.00 | 48.71 | C |
| ATOM | 10139 | CB | TRP | C | 368 | −3.063 | 40.534 | 11.486 | 1.00 | 84.38 | C |
| ATOM | 10140 | CG | TRP | C | 368 | −2.027 | 40.941 | 10.495 | 1.00 | 64.30 | C |
| ATOM | 10141 | CD2 | TRP | C | 368 | −0.996 | 41.916 | 10.688 | 1.00 | 64.30 | C |
| ATOM | 10142 | CE2 | TRP | C | 368 | −0.197 | 41.916 | 9.523 | 1.00 | 64.30 | C |
| ATOM | 10143 | CE3 | TRP | C | 368 | −0.669 | 42.788 | 11.731 | 1.00 | 64.30 | C |
| ATOM | 10144 | CD1 | TRP | C | 368 | −1.822 | 40.407 | 9.254 | 1.00 | 64.30 | C |
| ATOM | 10145 | NE1 | TRP | C | 368 | −0.724 | 40.986 | 8.663 | 1.00 | 64.30 | C |
| ATOM | 10146 | CZ2 | TRP | C | 368 | 0.911 | 42.757 | 9.374 | 1.00 | 64.30 | C |
| ATOM | 10147 | CZ3 | TRP | C | 368 | 0.431 | 43.621 | 11.581 | 1.00 | 64.30 | C |
| ATOM | 10148 | CH2 | TRP | C | 368 | 1.207 | 43.598 | 10.410 | 1.00 | 64.30 | C |
| ATOM | 10149 | C | TRP | C | 368 | −3.572 | 39.263 | 13.551 | 1.00 | 50.66 | C |
| ATOM | 10150 | O | TRP | C | 368 | −4.741 | 38.986 | 13.299 | 1.00 | 50.85 | C |
| ATOM | 10151 | N | ILE | C | 369 | −3.106 | 39.437 | 14.784 | 1.00 | 66.16 | C |
| ATOM | 10152 | CA | ILE | C | 369 | −3.951 | 39.265 | 15.955 | 1.00 | 67.53 | C |
| ATOM | 10153 | CB | ILE | C | 369 | −3.482 | 38.045 | 16.755 | 1.00 | 61.03 | C |
| ATOM | 10154 | CG2 | ILE | C | 369 | −4.398 | 37.815 | 17.939 | 1.00 | 38.71 | C |
| ATOM | 10155 | CG1 | ILE | C | 369 | −3.416 | 36.827 | 15.833 | 1.00 | 38.71 | C |
| ATOM | 10156 | CD1 | ILE | C | 369 | −2.812 | 35.617 | 16.475 | 1.00 | 38.71 | C |
| ATOM | 10157 | C | ILE | C | 369 | −3.910 | 40.487 | 16.859 | 1.00 | 69.35 | C |
| ATOM | 10158 | O | ILE | C | 369 | −2.840 | 41.037 | 17.109 | 1.00 | 66.15 | C |
| ATOM | 10159 | N | GLU | C | 370 | −5.073 | 40.917 | 17.344 | 1.00 | 67.72 | C |
| ATOM | 10160 | CA | GLU | C | 370 | −5.132 | 42.067 | 18.238 | 1.00 | 70.37 | C |
| ATOM | 10161 | CB | GLU | C | 370 | −6.581 | 42.375 | 18.593 | 1.00 | 115.11 | C |
| ATOM | 10162 | CG | GLU | C | 370 | −7.301 | 43.073 | 17.457 | 1.00 | 93.75 | C |
| ATOM | 10163 | CD | GLU | C | 370 | −8.794 | 43.158 | 17.661 | 1.00 | 93.75 | C |
| ATOM | 10164 | OE1 | GLU | C | 370 | −9.219 | 43.564 | 18.763 | 1.00 | 93.75 | C |
| ATOM | 10165 | OE2 | GLU | C | 370 | −9.542 | 42.826 | 16.715 | 1.00 | 93.75 | C |
| ATOM | 10166 | C | GLU | C | 370 | −4.309 | 41.745 | 19.478 | 1.00 | 70.55 | C |
| ATOM | 10167 | O | GLU | C | 370 | −4.440 | 40.667 | 20.052 | 1.00 | 69.40 | C |
| ATOM | 10168 | N | SER | C | 371 | −3.445 | 42.674 | 19.878 | 1.00 | 69.33 | C |
| ATOM | 10169 | CA | SER | C | 371 | −2.556 | 42.477 | 21.026 | 1.00 | 69.87 | C |
| ATOM | 10170 | CB | SER | C | 371 | −1.660 | 43.701 | 21.212 | 1.00 | 86.18 | C |
| ATOM | 10171 | OG | SER | C | 371 | −2.436 | 44.856 | 21.466 | 1.00 | 75.26 | C |
| ATOM | 10172 | C | SER | C | 371 | −3.221 | 42.146 | 22.356 | 1.00 | 69.14 | C |
| ATOM | 10173 | O | SER | C | 371 | −2.584 | 41.566 | 23.234 | 1.00 | 70.08 | C |
| ATOM | 10174 | N | GLU | C | 372 | −4.485 | 42.520 | 22.518 | 1.00 | 71.12 | C |
| ATOM | 10175 | CA | GLU | C | 372 | −5.193 | 42.229 | 23.760 | 1.00 | 69.67 | C |
| ATOM | 10176 | CB | GLU | C | 372 | −6.652 | 42.684 | 23.659 | 1.00 | 126.54 | C |
| ATOM | 10177 | CG | GLU | C | 372 | −7.407 | 42.054 | 22.502 | 1.00 | 126.54 | C |
| ATOM | 10178 | CD | GLU | C | 372 | −8.905 | 42.245 | 22.604 | 1.00 | 126.54 | C |
| ATOM | 10179 | OE1 | GLU | C | 372 | −9.500 | 41.740 | 23.580 | 1.00 | 126.54 | C |
| ATOM | 10180 | OE2 | GLU | C | 372 | −9.489 | 42.895 | 21.708 | 1.00 | 126.54 | C |
| ATOM | 10181 | C | GLU | C | 372 | −5.145 | 40.726 | 24.054 | 1.00 | 74.28 | C |
| ATOM | 10182 | O | GLU | C | 372 | −4.959 | 40.307 | 25.199 | 1.00 | 75.35 | C |
| ATOM | 10183 | N | HIS | C | 373 | −5.292 | 39.922 | 23.005 | 1.00 | 85.31 | C |
| ATOM | 10184 | CA | HIS | C | 373 | −5.298 | 38.474 | 23.132 | 1.00 | 88.80 | C |
| ATOM | 10185 | CB | HIS | C | 373 | −5.661 | 37.842 | 21.795 | 1.00 | 95.89 | C |
| ATOM | 10186 | CG | HIS | C | 373 | −7.026 | 38.210 | 21.318 | 1.00 | 65.24 | C |
| ATOM | 10187 | CD2 | HIS | C | 373 | −7.447 | 38.765 | 20.156 | 1.00 | 65.24 | C |
| ATOM | 10188 | ND1 | HIS | C | 373 | −8.153 | 38.024 | 22.086 | 1.00 | 65.24 | C |
| ATOM | 10189 | CE1 | HIS | C | 373 | −9.212 | 38.450 | 21.418 | 1.00 | 65.24 | C |
| ATOM | 10190 | NE2 | HIS | C | 373 | −8.810 | 38.905 | 20.244 | 1.00 | 65.24 | C |
| ATOM | 10191 | C | HIS | C | 373 | −4.011 | 37.859 | 23.649 | 1.00 | 84.57 | C |
| ATOM | 10192 | O | HIS | C | 373 | −3.974 | 36.665 | 23.939 | 1.00 | 80.41 | C |
| ATOM | 10193 | N | PHE | C | 374 | −2.953 | 38.651 | 23.766 | 1.00 | 91.88 | C |
| ATOM | 10194 | CA | PHE | C | 374 | −1.704 | 38.104 | 24.271 | 1.00 | 92.65 | C |
| ATOM | 10195 | CB | PHE | C | 374 | −0.515 | 38.618 | 23.467 | 1.00 | 85.43 | C |
| ATOM | 10196 | CG | PHE | C | 374 | −0.426 | 38.018 | 22.105 | 1.00 | 65.61 | C |
| ATOM | 10197 | CD1 | PHE | C | 374 | −1.198 | 38.514 | 21.062 | 1.00 | 65.61 | C |
| ATOM | 10198 | CD2 | PHE | C | 374 | 0.373 | 36.901 | 21.875 | 1.00 | 65.61 | C |
| ATOM | 10199 | CE1 | PHE | C | 374 | −1.182 | 37.907 | 19.815 | 1.00 | 65.61 | C |
| ATOM | 10200 | CE2 | PHE | C | 374 | 0.395 | 36.285 | 20.624 | 1.00 | 65.61 | C |
| ATOM | 10201 | CZ | PHE | C | 374 | −0.385 | 36.790 | 19.595 | 1.00 | 65.61 | C |
| ATOM | 10202 | C | PHE | C | 374 | −1.513 | 38.408 | 25.741 | 1.00 | 88.73 | C |
| ATOM | 10203 | O | PHE | C | 374 | −0.426 | 38.204 | 26.291 | 1.00 | 86.97 | C |
| ATOM | 10204 | N | LYS | C | 375 | −2.587 | 38.881 | 26.369 | 1.00 | 128.27 | C |
| ATOM | 10205 | CA | LYS | C | 375 | −2.595 | 39.213 | 27.790 | 1.00 | 130.90 | C |
| ATOM | 10206 | CB | LYS | C | 375 | −3.213 | 38.063 | 28.600 | 1.00 | 138.04 | C |
| ATOM | 10207 | CG | LYS | C | 375 | −4.741 | 38.005 | 28.612 | 1.00 | 111.88 | C |
| ATOM | 10208 | CD | LYS | C | 375 | −5.327 | 37.599 | 27.268 | 1.00 | 111.88 | C |
| ATOM | 10209 | CE | LYS | C | 375 | −6.850 | 37.455 | 27.350 | 1.00 | 111.88 | C |
| ATOM | 10210 | NZ | LYS | C | 375 | −7.484 | 37.104 | 26.036 | 1.00 | 111.88 | C |
| ATOM | 10211 | C | LYS | C | 375 | −1.219 | 39.544 | 28.365 | 1.00 | 135.92 | C |
| ATOM | 10212 | O | LYS | C | 375 | −0.787 | 38.926 | 29.336 | 1.00 | 138.18 | C |
| ATOM | 10213 | N | GLY | C | 376 | −0.527 | 40.504 | 27.761 | 1.00 | 87.98 | C |
| ATOM | 10214 | CA | GLY | C | 376 | 0.779 | 40.895 | 28.271 | 1.00 | 87.98 | C |
| ATOM | 10215 | C | GLY | C | 376 | 2.003 | 40.098 | 27.839 | 1.00 | 87.98 | C |
| ATOM | 10216 | O | GLY | C | 376 | 3.108 | 40.646 | 27.783 | 1.00 | 87.98 | C |
| ATOM | 10217 | N | GLU | C | 377 | 1.834 | 38.809 | 27.555 | 1.00 | 111.16 | C |
| ATOM | 10218 | CA | GLU | C | 377 | 2.962 | 37.990 | 27.125 | 1.00 | 111.16 | C |
| ATOM | 10219 | CB | GLU | C | 377 | 2.946 | 36.635 | 27.836 | 1.00 | 104.19 | C |
| ATOM | 10220 | CG | GLU | C | 377 | 4.225 | 35.832 | 27.640 | 1.00 | 104.19 | C |
| ATOM | 10221 | CD | GLU | C | 377 | 4.205 | 34.506 | 28.381 | 1.00 | 104.19 | C |
| ATOM | 10222 | OE1 | GLU | C | 377 | 5.254 | 33.823 | 28.419 | 1.00 | 104.19 | C |
| ATOM | 10223 | OE2 | GLU | C | 377 | 3.135 | 34.148 | 28.922 | 1.00 | 104.19 | C |
| ATOM | 10224 | C | GLU | C | 377 | 2.891 | 37.787 | 25.613 | 1.00 | 111.16 | C |
| ATOM | 10225 | O | GLU | C | 377 | 1.934 | 37.198 | 25.103 | 1.00 | 111.16 | C |
| ATOM | 10226 | N | TYR | C | 378 | 3.905 | 38.278 | 24.901 | 1.00 | 63.91 | C |
| ATOM | 10227 | CA | TYR | C | 378 | 3.935 | 38.162 | 23.444 | 1.00 | 63.91 | C |
| ATOM | 10228 | CB | TYR | C | 378 | 4.569 | 39.409 | 22.834 | 1.00 | 78.86 | C |
| ATOM | 10229 | CG | TYR | C | 378 | 3.684 | 40.617 | 23.002 | 1.00 | 78.86 | C |
| ATOM | 10230 | CD1 | TYR | C | 378 | 3.848 | 41.486 | 24.080 | 1.00 | 78.86 | C |
| ATOM | 10231 | CE1 | TYR | C | 378 | 2.972 | 42.557 | 24.287 | 1.00 | 78.86 | C |
| ATOM | 10232 | CD2 | TYR | C | 378 | 2.625 | 40.850 | 22.127 | 1.00 | 78.86 | C |
| ATOM | 10233 | CE2 | TYR | C | 378 | 1.745 | 41.914 | 22.325 | 1.00 | 78.86 | C |
| ATOM | 10234 | CZ | TYR | C | 378 | 1.924 | 42.762 | 23.408 | 1.00 | 78.86 | C |
| ATOM | 10235 | OH | TYR | C | 378 | 1.044 | 43.799 | 23.615 | 1.00 | 78.86 | C |
| ATOM | 10236 | C | TYR | C | 378 | 4.585 | 36.906 | 22.868 | 1.00 | 63.91 | C |
| ATOM | 10237 | O | TYR | C | 378 | 5.622 | 36.968 | 22.188 | 1.00 | 63.91 | C |
| ATOM | 10238 | N | LYS | C | 379 | 3.944 | 35.771 | 23.156 | 1.00 | 62.49 | C |
| ATOM | 10239 | CA | LYS | C | 379 | 4.361 | 34.456 | 22.678 | 1.00 | 62.49 | C |
| ATOM | 10240 | CB | LYS | C | 379 | 4.957 | 33.616 | 23.808 | 1.00 | 74.16 | C |
| ATOM | 10241 | CG | LYS | C | 379 | 6.272 | 34.141 | 24.365 | 1.00 | 74.16 | C |
| ATOM | 10242 | CD | LYS | C | 379 | 6.754 | 33.251 | 25.504 | 1.00 | 74.16 | C |
| ATOM | 10243 | CE | LYS | C | 379 | 7.985 | 33.815 | 26.215 | 1.00 | 74.16 | C |
| ATOM | 10244 | NZ | LYS | C | 379 | 8.325 | 33.024 | 27.438 | 1.00 | 74.16 | C |
| ATOM | 10245 | C | LYS | C | 379 | 3.088 | 33.799 | 22.165 | 1.00 | 62.49 | C |
| ATOM | 10246 | O | LYS | C | 379 | 2.021 | 33.928 | 22.768 | 1.00 | 62.49 | C |
| ATOM | 10247 | N | TYR | C | 380 | 3.191 | 33.123 | 21.033 | 1.00 | 43.47 | C |
| ATOM | 10248 | CA | TYR | C | 380 | 2.037 | 32.453 | 20.449 | 1.00 | 43.47 | C |
| ATOM | 10249 | CB | TYR | C | 380 | 2.198 | 32.393 | 18.934 | 1.00 | 54.94 | C |
| ATOM | 10250 | CG | TYR | C | 380 | 1.024 | 31.795 | 18.213 | 1.00 | 54.94 | C |
| ATOM | 10251 | CD1 | TYR | C | 380 | −0.058 | 32.584 | 17.845 | 1.00 | 54.94 | C |
| ATOM | 10252 | CE1 | TYR | C | 380 | −1.140 | 32.040 | 17.166 | 1.00 | 54.94 | C |
| ATOM | 10253 | CD2 | TYR | C | 380 | 0.997 | 30.436 | 17.889 | 1.00 | 54.94 | C |
| ATOM | 10254 | CE2 | TYR | C | 380 | −0.084 | 29.881 | 17.216 | 1.00 | 54.94 | C |
| ATOM | 10255 | CZ | TYR | C | 380 | −1.147 | 30.687 | 16.855 | 1.00 | 54.94 | C |
| ATOM | 10256 | OH | TYR | C | 380 | −2.214 | 30.142 | 16.177 | 1.00 | 54.94 | C |
| ATOM | 10257 | C | TYR | C | 380 | 2.030 | 31.049 | 21.025 | 1.00 | 43.47 | C |
| ATOM | 10258 | O | TYR | C | 380 | 2.932 | 30.260 | 20.735 | 1.00 | 43.47 | C |
| ATOM | 10259 | N | VAL | C | 381 | 1.029 | 30.742 | 21.845 | 1.00 | 49.05 | C |
| ATOM | 10260 | CA | VAL | C | 381 | 0.935 | 29.417 | 22.471 | 1.00 | 49.05 | C |
| ATOM | 10261 | CB | VAL | C | 381 | 0.114 | 29.477 | 23.779 | 1.00 | 53.25 | C |
| ATOM | 10262 | CG1 | VAL | C | 381 | 0.130 | 28.122 | 24.459 | 1.00 | 53.25 | C |
| ATOM | 10263 | CG2 | VAL | C | 381 | 0.688 | 30.535 | 24.702 | 1.00 | 53.25 | C |
| ATOM | 10264 | C | VAL | C | 381 | 0.296 | 28.409 | 21.515 | 1.00 | 49.05 | C |
| ATOM | 10265 | O | VAL | C | 381 | −0.840 | 28.595 | 21.087 | 1.00 | 49.05 | C |
| ATOM | 10266 | N | ASN | C | 382 | 1.029 | 27.340 | 21.210 | 1.00 | 71.19 | C |
| ATOM | 10267 | CA | ASN | C | 382 | 0.572 | 26.328 | 20.270 | 1.00 | 71.19 | C |
| ATOM | 10268 | CB | ASN | C | 382 | 1.335 | 25.019 | 20.459 | 1.00 | 51.24 | C |
| ATOM | 10269 | CG | ASN | C | 382 | 1.355 | 24.152 | 19.179 | 1.00 | 51.24 | C |
| ATOM | 10270 | OD1 | ASN | C | 382 | 0.418 | 24.166 | 18.380 | 1.00 | 51.24 | C |
| ATOM | 10271 | ND2 | ASN | C | 382 | 2.423 | 23.381 | 19.006 | 1.00 | 51.24 | C |
| ATOM | 10272 | C | ASN | C | 382 | −0.917 | 26.014 | 20.270 | 1.00 | 71.19 | C |
| ATOM | 10273 | O | ASN | C | 382 | −1.667 | 26.487 | 19.408 | 1.00 | 71.19 | C |
| ATOM | 10274 | N | GLY | C | 383 | −1.365 | 25.214 | 21.225 | 1.00 | 39.39 | C |
| ATOM | 10275 | CA | GLY | C | 383 | −2.776 | 24.861 | 21.211 | 1.00 | 39.39 | C |
| ATOM | 10276 | C | GLY | C | 383 | −3.802 | 25.923 | 21.573 | 1.00 | 39.39 | C |
| ATOM | 10277 | O | GLY | C | 383 | −4.992 | 25.649 | 21.588 | 1.00 | 39.39 | C |
| ATOM | 10278 | N | ARG | C | 384 | −3.366 | 27.139 | 21.858 | 1.00 | 46.21 | C |
| ATOM | 10279 | CA | ARG | C | 384 | −4.302 | 28.182 | 22.256 | 1.00 | 46.21 | C |
| ATOM | 10280 | CB | ARG | C | 384 | −3.540 | 29.317 | 22.942 | 1.00 | 52.28 | C |
| ATOM | 10281 | CG | ARG | C | 384 | −4.419 | 30.379 | 23.572 | 1.00 | 52.28 | C |
| ATOM | 10282 | CD | ARG | C | 384 | −3.604 | 31.210 | 24.541 | 1.00 | 52.28 | C |
| ATOM | 10283 | NE | ARG | C | 384 | −3.036 | 30.372 | 25.585 | 1.00 | 52.28 | C |
| ATOM | 10284 | CZ | ARG | C | 384 | −2.133 | 30.776 | 26.471 | 1.00 | 52.28 | C |
| ATOM | 10285 | NH1 | ARG | C | 384 | −1.690 | 32.016 | 26.443 | 1.00 | 52.28 | C |
| ATOM | 10286 | NH2 | ARG | C | 384 | −1.667 | 29.931 | 27.386 | 1.00 | 52.28 | C |
| ATOM | 10287 | C | ARG | C | 384 | −5.123 | 28.739 | 21.115 | 1.00 | 46.21 | C |
| ATOM | 10288 | O | ARG | C | 384 | −4.670 | 28.784 | 19.972 | 1.00 | 46.21 | C |
| ATOM | 10289 | N | TYR | C | 385 | −6.337 | 29.165 | 21.435 | 1.00 | 52.85 | C |
| ATOM | 10290 | CA | TYR | C | 385 | −7.223 | 29.742 | 20.432 | 1.00 | 52.85 | C |
| ATOM | 10291 | CB | TYR | C | 385 | −8.686 | 29.522 | 20.823 | 1.00 | 56.05 | C |
| ATOM | 10292 | CG | TYR | C | 385 | −9.636 | 30.281 | 19.937 | 1.00 | 56.05 | C |
| ATOM | 10293 | CD1 | TYR | C | 385 | −9.902 | 29.842 | 18.638 | 1.00 | 56.05 | C |
| ATOM | 10294 | CE1 | TYR | C | 385 | −10.706 | 30.581 | 17.779 | 1.00 | 56.05 | C |
| ATOM | 10295 | CD2 | TYR | C | 385 | −10.207 | 31.484 | 20.362 | 1.00 | 56.05 | C |
| ATOM | 10296 | CE2 | TYR | C | 385 | −11.012 | 32.238 | 19.514 | 1.00 | 56.05 | C |
| ATOM | 10297 | CZ | TYR | C | 385 | −11.257 | 31.779 | 18.217 | 1.00 | 56.05 | C |
| ATOM | 10298 | OH | TYR | C | 385 | −12.037 | 32.519 | 17.350 | 1.00 | 56.05 | C |
| ATOM | 10299 | C | TYR | C | 385 | −6.971 | 31.252 | 20.285 | 1.00 | 52.85 | C |
| ATOM | 10300 | O | TYR | C | 385 | −6.717 | 31.942 | 21.276 | 1.00 | 52.85 | C |
| ATOM | 10301 | N | TYR | C | 386 | −7.043 | 31.764 | 19.057 | 1.00 | 51.34 | C |
| ATOM | 10302 | CA | TYR | C | 386 | −6.839 | 33.196 | 18.814 | 1.00 | 51.34 | C |
| ATOM | 10303 | CB | TYR | C | 386 | −5.390 | 33.519 | 18.410 | 1.00 | 44.13 | C |
| ATOM | 10304 | CG | TYR | C | 386 | −4.316 | 33.232 | 19.423 | 1.00 | 44.13 | C |
| ATOM | 10305 | CD1 | TYR | C | 386 | −3.591 | 32.044 | 19.372 | 1.00 | 44.13 | C |
| ATOM | 10306 | CE1 | TYR | C | 386 | −2.555 | 31.794 | 20.273 | 1.00 | 44.13 | C |
| ATOM | 10307 | CD2 | TYR | C | 386 | −3.986 | 34.166 | 20.405 | 1.00 | 44.13 | C |
| ATOM | 10308 | CE2 | TYR | C | 386 | −2.953 | 33.930 | 21.308 | 1.00 | 44.13 | C |
| ATOM | 10309 | CZ | TYR | C | 386 | −2.237 | 32.744 | 21.237 | 1.00 | 44.13 | C |
| ATOM | 10310 | OH | TYR | C | 386 | −1.179 | 32.509 | 22.094 | 1.00 | 44.13 | C |
| ATOM | 10311 | C | TYR | C | 386 | −7.707 | 33.720 | 17.677 | 1.00 | 51.34 | C |
| ATOM | 10312 | O | TYR | C | 386 | −7.675 | 33.181 | 16.575 | 1.00 | 51.34 | C |
| ATOM | 10313 | N | PRO | C | 387 | −8.507 | 34.766 | 17.931 | 1.00 | 64.40 | C |
| ATOM | 10314 | CD | PRO | C | 387 | −8.883 | 35.338 | 19.234 | 1.00 | 82.91 | C |
| ATOM | 10315 | CA | PRO | C | 387 | −9.338 | 35.317 | 16.851 | 1.00 | 64.40 | C |
| ATOM | 10316 | CB | PRO | C | 387 | −10.142 | 36.393 | 17.562 | 1.00 | 82.91 | C |
| ATOM | 10317 | CG | PRO | C | 387 | −10.271 | 35.841 | 18.957 | 1.00 | 82.91 | C |
| ATOM | 10318 | C | PRO | C | 387 | −8.269 | 35.906 | 15.921 | 1.00 | 64.40 | C |
| ATOM | 10319 | O | PRO | C | 387 | −7.257 | 36.397 | 16.423 | 1.00 | 64.40 | C |
| ATOM | 10320 | N | GLU | C | 388 | −8.445 | 35.897 | 14.601 | 1.00 | 79.00 | C |
| ATOM | 10321 | CA | GLU | C | 388 | −7.329 | 36.394 | 13.814 | 1.00 | 79.00 | C |
| ATOM | 10322 | CB | GLU | C | 388 | −6.483 | 35.208 | 13.361 | 1.00 | 108.73 | C |
| ATOM | 10323 | CG | GLU | C | 388 | −7.147 | 34.332 | 12.324 | 1.00 | 108.73 | C |
| ATOM | 10324 | CD | GLU | C | 388 | −6.176 | 33.346 | 11.705 | 1.00 | 108.73 | C |
| ATOM | 10325 | OE1 | GLU | C | 388 | −5.161 | 33.798 | 11.138 | 1.00 | 108.73 | C |
| ATOM | 10326 | OE2 | GLU | C | 388 | −6.423 | 32.124 | 11.784 | 1.00 | 108.73 | C |
| ATOM | 10327 | C | GLU | C | 388 | −7.400 | 37.361 | 12.646 | 1.00 | 79.00 | C |
| ATOM | 10328 | O | GLU | C | 388 | −6.450 | 37.396 | 11.861 | 1.00 | 79.00 | C |
| ATOM | 10329 | N | TRP | C | 389 | −8.456 | 38.149 | 12.492 | 1.00 | 63.56 | C |
| ATOM | 10330 | CA | TRP | C | 389 | −8.455 | 39.102 | 11.369 | 1.00 | 63.56 | C |
| ATOM | 10331 | CB | TRP | C | 389 | −7.391 | 40.186 | 11.593 | 1.00 | 116.25 | C |
| ATOM | 10332 | CG | TRP | C | 389 | −7.875 | 41.473 | 12.179 | 1.00 | 116.25 | C |
| ATOM | 10333 | CD2 | TRP | C | 389 | −9.238 | 41.869 | 12.410 | 1.00 | 116.25 | C |
| ATOM | 10334 | CE2 | TRP | C | 389 | −9.205 | 43.174 | 12.950 | 1.00 | 116.25 | C |
| ATOM | 10335 | CE3 | TRP | C | 389 | −10.485 | 41.250 | 12.211 | 1.00 | 116.25 | C |
| ATOM | 10336 | CD1 | TRP | C | 389 | −7.099 | 42.524 | 12.579 | 1.00 | 116.25 | C |
| ATOM | 10337 | NE1 | TRP | C | 389 | −7.888 | 43.548 | 13.042 | 1.00 | 116.25 | C |
| ATOM | 10338 | CZ2 | TRP | C | 389 | −10.371 | 43.873 | 13.296 | 1.00 | 116.25 | C |
| ATOM | 10339 | CZ3 | TRP | C | 389 | −11.644 | 41.946 | 12.554 | 1.00 | 116.25 | C |
| ATOM | 10340 | CH2 | TRP | C | 389 | −11.576 | 43.244 | 13.090 | 1.00 | 116.25 | C |
| ATOM | 10341 | C | TRP | C | 389 | −8.143 | 38.424 | 10.036 | 1.00 | 63.56 | C |
| ATOM | 10342 | O | TRP | C | 389 | −7.056 | 38.618 | 9.479 | 1.00 | 63.56 | C |
| ATOM | 10343 | N | GLY | C | 390 | −9.085 | 37.642 | 9.520 | 1.00 | 75.39 | C |
| ATOM | 10344 | CA | GLY | C | 390 | −8.845 | 36.961 | 8.262 | 1.00 | 75.39 | C |
| ATOM | 10345 | C | GLY | C | 390 | −8.639 | 37.879 | 7.067 | 1.00 | 75.39 | C |
| ATOM | 10346 | O | GLY | C | 390 | −9.092 | 39.021 | 7.067 | 1.00 | 75.39 | C |
| ATOM | 10347 | N | ARG | C | 391 | −7.936 | 37.377 | 6.053 | 1.00 | 89.36 | C |
| ATOM | 10348 | CA | ARG | C | 391 | −7.694 | 38.126 | 4.824 | 1.00 | 89.36 | C |
| ATOM | 10349 | CB | ARG | C | 391 | −6.335 | 37.738 | 4.233 | 1.00 | 104.79 | C |
| ATOM | 10350 | CG | ARG | C | 391 | −5.152 | 37.981 | 5.171 | 1.00 | 104.79 | C |
| ATOM | 10351 | CD | ARG | C | 391 | −4.449 | 39.326 | 4.921 | 1.00 | 104.79 | C |
| ATOM | 10352 | NE | ARG | C | 391 | −3.356 | 39.227 | 3.947 | 1.00 | 104.79 | C |
| ATOM | 10353 | CZ | ARG | C | 391 | −3.513 | 39.149 | 2.624 | 1.00 | 104.79 | C |
| ATOM | 10354 | NH1 | ARG | C | 391 | −4.728 | 39.163 | 2.090 | 1.00 | 104.79 | C |
| ATOM | 10355 | NH2 | ARG | C | 391 | −2.448 | 39.046 | 1.833 | 1.00 | 104.79 | C |
| ATOM | 10356 | C | ARG | C | 391 | −8.825 | 37.742 | 3.860 | 1.00 | 89.36 | C |
| ATOM | 10357 | O | ARG | C | 391 | −9.551 | 36.779 | 4.110 | 1.00 | 89.36 | C |
| ATOM | 10358 | N | LYS | C | 392 | −8.987 | 38.488 | 2.771 | 1.00 | 73.58 | C |
| ATOM | 10359 | CA | LYS | C | 392 | −10.046 | 38.188 | 1.806 | 1.00 | 73.58 | C |
| ATOM | 10360 | CB | LYS | C | 392 | −10.892 | 39.434 | 1.532 | 1.00 | 94.02 | C |
| ATOM | 10361 | CG | LYS | C | 392 | −12.021 | 39.191 | 0.551 | 1.00 | 94.02 | C |
| ATOM | 10362 | CD | LYS | C | 392 | −12.685 | 40.483 | 0.124 | 1.00 | 94.02 | C |
| ATOM | 10363 | CE | LYS | C | 392 | −13.687 | 40.237 | −0.998 | 1.00 | 94.02 | C |
| ATOM | 10364 | NZ | LYS | C | 392 | −14.209 | 41.513 | −1.571 | 1.00 | 94.02 | C |
| ATOM | 10365 | C | LYS | C | 392 | −9.472 | 37.661 | 0.493 | 1.00 | 73.58 | C |
| ATOM | 10366 | O | LYS | C | 392 | −8.763 | 38.374 | −0.213 | 1.00 | 73.58 | C |
| ATOM | 10367 | N | SER | C | 393 | −9.781 | 36.406 | 0.177 | 1.00 | 65.87 | C |
| ATOM | 10368 | CA | SER | C | 393 | −9.297 | 35.774 | −1.045 | 1.00 | 65.87 | C |
| ATOM | 10369 | CB | SER | C | 393 | −7.815 | 35.394 | −0.899 | 1.00 | 73.44 | C |
| ATOM | 10370 | OG | SER | C | 393 | −7.342 | 34.706 | −2.052 | 1.00 | 73.44 | C |
| ATOM | 10371 | C | SER | C | 393 | −10.114 | 34.523 | −1.330 | 1.00 | 65.87 | C |
| ATOM | 10372 | O | SER | C | 393 | −10.742 | 33.977 | −0.429 | 1.00 | 65.87 | C |
| ATOM | 10373 | N | ASN | C | 394 | −10.114 | 34.073 | −2.580 | 1.00 | 65.79 | C |
| ATOM | 10374 | CA | ASN | C | 394 | −10.848 | 32.866 | −2.928 | 1.00 | 65.79 | C |
| ATOM | 10375 | CB | ASN | C | 394 | −11.073 | 32.778 | −4.427 | 1.00 | 71.81 | C |
| ATOM | 10376 | CG | ASN | C | 394 | −12.084 | 33.783 | −4.909 | 1.00 | 71.81 | C |
| ATOM | 10377 | OD1 | ASN | C | 394 | −13.175 | 33.903 | −4.343 | 1.00 | 71.81 | C |
| ATOM | 10378 | ND2 | ASN | C | 394 | −11.734 | 34.514 | −5.963 | 1.00 | 71.81 | C |
| ATOM | 10379 | C | ASN | C | 394 | −10.060 | 31.661 | −2.464 | 1.00 | 65.79 | C |
| ATOM | 10380 | O | ASN | C | 394 | −10.595 | 30.554 | −2.344 | 1.00 | 65.79 | C |
| ATOM | 10381 | N | TYR | C | 395 | −8.777 | 31.892 | −2.207 | 1.00 | 53.78 | C |
| ATOM | 10382 | CA | TYR | C | 395 | −7.889 | 30.847 | −1.734 | 1.00 | 53.78 | C |
| ATOM | 10383 | CB | TYR | C | 395 | −6.515 | 30.997 | −2.379 | 1.00 | 60.39 | C |
| ATOM | 10384 | CG | TYR | C | 395 | −6.518 | 30.818 | −3.875 | 1.00 | 60.39 | C |
| ATOM | 10385 | CD1 | TYR | C | 395 | −6.629 | 31.911 | −4.725 | 1.00 | 60.39 | C |
| ATOM | 10386 | CE1 | TYR | C | 395 | −6.614 | 31.747 | −6.112 | 1.00 | 60.39 | C |
| ATOM | 10387 | CD2 | TYR | C | 395 | −6.397 | 29.544 | −4.445 | 1.00 | 60.39 | C |
| ATOM | 10388 | CE2 | TYR | C | 395 | −6.380 | 29.371 | −5.825 | 1.00 | 60.39 | C |
| ATOM | 10389 | CZ | TYR | C | 395 | −6.489 | 30.481 | −6.651 | 1.00 | 60.39 | C |
| ATOM | 10390 | OH | TYR | C | 395 | −6.481 | 30.317 | −8.012 | 1.00 | 60.39 | C |
| ATOM | 10391 | C | TYR | C | 395 | −7.753 | 30.943 | −0.224 | 1.00 | 53.78 | C |
| ATOM | 10392 | O | TYR | C | 395 | −7.971 | 32.008 | 0.352 | 1.00 | 53.78 | C |
| ATOM | 10393 | N | GLU | C | 396 | −7.409 | 29.825 | 0.412 | 1.00 | 66.90 | C |
| ATOM | 10394 | CA | GLU | C | 396 | −7.210 | 29.778 | 1.860 | 1.00 | 66.90 | C |
| ATOM | 10395 | CB | GLU | C | 396 | −7.828 | 28.512 | 2.451 | 1.00 | 94.83 | C |
| ATOM | 10396 | CG | GLU | C | 396 | −9.250 | 28.229 | 2.006 | 1.00 | 94.83 | C |
| ATOM | 10397 | CD | GLU | C | 396 | −9.712 | 26.838 | 2.418 | 1.00 | 94.83 | C |
| ATOM | 10398 | OE1 | GLU | C | 396 | −9.000 | 25.861 | 2.092 | 1.00 | 94.83 | C |
| ATOM | 10399 | OE2 | GLU | C | 396 | −10.781 | 26.720 | 3.062 | 1.00 | 94.83 | C |
| ATOM | 10400 | C | GLU | C | 396 | −5.702 | 29.730 | 2.068 | 1.00 | 66.90 | C |
| ATOM | 10401 | O | GLU | C | 396 | −5.002 | 29.038 | 1.327 | 1.00 | 66.90 | C |
| ATOM | 10402 | N | PRO | C | 397 | −5.176 | 30.467 | 3.064 | 1.00 | 55.43 | C |
| ATOM | 10403 | CD | PRO | C | 397 | −5.845 | 31.251 | 4.115 | 1.00 | 63.83 | C |
| ATOM | 10404 | CA | PRO | C | 397 | −3.723 | 30.431 | 3.286 | 1.00 | 55.43 | C |
| ATOM | 10405 | CB | PRO | C | 397 | −3.548 | 31.213 | 4.586 | 1.00 | 63.83 | C |
| ATOM | 10406 | CG | PRO | C | 397 | −4.889 | 31.096 | 5.262 | 1.00 | 63.83 | C |
| ATOM | 10407 | C | PRO | C | 397 | −3.234 | 28.995 | 3.383 | 1.00 | 55.43 | C |
| ATOM | 10408 | O | PRO | C | 397 | −3.823 | 28.171 | 4.080 | 1.00 | 55.43 | C |
| ATOM | 10409 | N | GLY | C | 398 | −2.158 | 28.699 | 2.672 | 1.00 | 47.53 | C |
| ATOM | 10410 | CA | GLY | C | 398 | −1.649 | 27.343 | 2.654 | 1.00 | 47.53 | C |
| ATOM | 10411 | C | GLY | C | 398 | −1.703 | 26.852 | 1.217 | 1.00 | 47.53 | C |
| ATOM | 10412 | O | GLY | C | 398 | −0.763 | 26.182 | 0.757 | 1.00 | 47.53 | C |
| ATOM | 10413 | N | GLU | C | 399 | −2.794 | 27.181 | 0.509 | 1.00 | 48.60 | C |
| ATOM | 10414 | CA | GLU | C | 399 | −2.953 | 26.784 | −0.900 | 1.00 | 48.60 | C |
| ATOM | 10415 | CB | GLU | C | 399 | −4.389 | 27.026 | −1.383 | 1.00 | 67.07 | C |
| ATOM | 10416 | CG | GLU | C | 399 | −5.446 | 26.365 | −0.523 | 1.00 | 67.07 | C |
| ATOM | 10417 | CD | GLU | C | 399 | −6.863 | 26.574 | −1.033 | 1.00 | 67.07 | C |
| ATOM | 10418 | OE1 | GLU | C | 399 | −7.188 | 27.710 | −1.431 | 1.00 | 67.07 | C |
| ATOM | 10419 | OE2 | GLU | C | 399 | −7.664 | 25.612 | −1.017 | 1.00 | 67.07 | C |
| ATOM | 10420 | C | GLU | C | 399 | −1.981 | 27.649 | −1.709 | 1.00 | 48.60 | C |
| ATOM | 10421 | O | GLU | C | 399 | −1.869 | 28.846 | −1.460 | 1.00 | 48.60 | C |
| ATOM | 10422 | N | PRO | C | 400 | −1.254 | 27.061 | −2.673 | 1.00 | 75.85 | C |
| ATOM | 10423 | CD | PRO | C | 400 | −1.163 | 25.666 | −3.123 | 1.00 | 47.83 | C |
| ATOM | 10424 | CA | PRO | C | 400 | −0.330 | 27.907 | −3.423 | 1.00 | 70.93 | C |
| ATOM | 10425 | CB | PRO | C | 400 | 0.307 | 26.940 | −4.420 | 1.00 | 96.44 | C |
| ATOM | 10426 | CG | PRO | C | 400 | −0.667 | 25.835 | −4.520 | 1.00 | 47.83 | C |
| ATOM | 10427 | C | PRO | C | 400 | −0.979 | 29.114 | −4.081 | 1.00 | 61.68 | C |
| ATOM | 10428 | O | PRO | C | 400 | −0.300 | 30.084 | −4.417 | 1.00 | 61.89 | C |
| ATOM | 10429 | N | GLY | C | 401 | −2.294 | 29.078 | −4.243 | 1.00 | 50.15 | C |
| ATOM | 10430 | CA | GLY | C | 401 | −2.974 | 30.211 | −4.855 | 1.00 | 49.71 | C |
| ATOM | 10431 | C | GLY | C | 401 | −3.151 | 31.437 | −3.962 | 1.00 | 49.50 | C |
| ATOM | 10432 | O | GLY | C | 401 | −3.590 | 32.487 | −4.429 | 1.00 | 50.21 | C |
| ATOM | 10433 | N | PHE | C | 402 | −2.821 | 31.326 | −2.678 | 1.00 | 59.61 | C |
| ATOM | 10434 | CA | PHE | C | 402 | −2.978 | 32.466 | −1.788 | 1.00 | 59.61 | C |
| ATOM | 10435 | CB | PHE | C | 402 | −2.811 | 32.047 | −0.337 | 1.00 | 156.44 | C |
| ATOM | 10436 | CG | PHE | C | 402 | −3.425 | 33.009 | 0.624 | 1.00 | 54.77 | C |
| ATOM | 10437 | CD1 | PHE | C | 402 | −4.804 | 33.036 | 0.805 | 1.00 | 54.77 | C |
| ATOM | 10438 | CD2 | PHE | C | 402 | −2.635 | 33.923 | 1.314 | 1.00 | 54.77 | C |
| ATOM | 10439 | CE1 | PHE | C | 402 | −5.395 | 33.961 | 1.658 | 1.00 | 54.77 | C |
| ATOM | 10440 | CE2 | PHE | C | 402 | −3.209 | 34.852 | 2.167 | 1.00 | 54.77 | C |
| ATOM | 10441 | CZ | PHE | C | 402 | −4.596 | 34.873 | 2.340 | 1.00 | 54.77 | C |
| ATOM | 10442 | C | PHE | C | 402 | −1.956 | 33.550 | −2.128 | 1.00 | 59.61 | C |
| ATOM | 10443 | O | PHE | C | 402 | −0.762 | 33.267 | −2.257 | 1.00 | 59.61 | C |
| ATOM | 10444 | N | PRO | C | 403 | −2.418 | 34.809 | −2.273 | 1.00 | 93.77 | C |
| ATOM | 10445 | CD | PRO | C | 403 | −3.828 | 35.200 | −2.141 | 1.00 | 49.25 | C |
| ATOM | 10446 | CA | PRO | C | 403 | −1.602 | 35.983 | −2.602 | 1.00 | 93.77 | C |
| ATOM | 10447 | CB | PRO | C | 403 | −2.637 | 37.074 | −2.884 | 1.00 | 154.08 | C |
| ATOM | 10448 | CG | PRO | C | 403 | −3.905 | 36.344 | −3.097 | 1.00 | 49.25 | C |
| ATOM | 10449 | C | PRO | C | 403 | −0.667 | 36.425 | −1.488 | 1.00 | 93.77 | C |
| ATOM | 10450 | O | PRO | C | 403 | −1.099 | 37.070 | −0.530 | 1.00 | 93.77 | C |
| ATOM | 10451 | N | TRP | C | 404 | 0.607 | 36.076 | −1.606 | 1.00 | 73.97 | C |
| ATOM | 10452 | CA | TRP | C | 404 | 1.597 | 36.490 | −0.619 | 1.00 | 73.97 | C |
| ATOM | 10453 | CB | TRP | C | 404 | 2.314 | 35.290 | 0.016 | 1.00 | 63.24 | C |
| ATOM | 10454 | CG | TRP | C | 404 | 1.516 | 34.526 | 1.040 | 1.00 | 56.48 | C |
| ATOM | 10455 | CD2 | TRP | C | 404 | 0.985 | 35.022 | 2.284 | 1.00 | 56.48 | C |
| ATOM | 10456 | CE2 | TRP | C | 404 | 0.343 | 33.943 | 2.921 | 1.00 | 56.48 | C |
| ATOM | 10457 | CE3 | TRP | C | 404 | 0.992 | 36.271 | 2.918 | 1.00 | 56.48 | C |
| ATOM | 10458 | CD1 | TRP | C | 404 | 1.179 | 33.211 | 0.984 | 1.00 | 56.48 | C |
| ATOM | 10459 | NE1 | TRP | C | 404 | 0.477 | 32.851 | 2.106 | 1.00 | 56.48 | C |
| ATOM | 10460 | CZ2 | TRP | C | 404 | −0.288 | 34.070 | 4.164 | 1.00 | 56.48 | C |
| ATOM | 10461 | CZ3 | TRP | C | 404 | 0.362 | 36.398 | 4.162 | 1.00 | 56.48 | C |
| ATOM | 10462 | CH2 | TRP | C | 404 | −0.267 | 35.301 | 4.766 | 1.00 | 56.48 | C |
| ATOM | 10463 | C | TRP | C | 404 | 2.600 | 37.307 | −1.407 | 1.00 | 73.97 | C |
| ATOM | 10464 | O | TRP | C | 404 | 3.072 | 36.861 | −2.452 | 1.00 | 73.97 | C |
| ATOM | 10465 | N | ASN | C | 405 | 2.923 | 38.501 | −0.922 | 1.00 | 62.25 | C |
| ATOM | 10466 | CA | ASN | C | 405 | 3.879 | 39.347 | −1.624 | 1.00 | 62.28 | C |
| ATOM | 10467 | CB | ASN | C | 405 | 3.718 | 40.799 | −1.185 | 1.00 | 84.75 | C |
| ATOM | 10468 | CG | ASN | C | 405 | 4.265 | 41.767 | −2.202 | 1.00 | 84.75 | C |
| ATOM | 10469 | OD1 | ASN | C | 405 | 5.476 | 41.871 | −2.397 | 1.00 | 84.75 | C |
| ATOM | 10470 | ND2 | ASN | C | 405 | 3.369 | 42.474 | −2.875 | 1.00 | 84.75 | C |
| ATOM | 10471 | C | ASN | C | 405 | 5.303 | 38.866 | −1.359 | 1.00 | 71.22 | C |
| ATOM | 10472 | O | ASN | C | 405 | 6.056 | 39.467 | −0.588 | 1.00 | 64.28 | C |
| ATOM | 10473 | N | ILE | C | 406 | 5.657 | 37.762 | −2.000 | 1.00 | 61.33 | C |
| ATOM | 10474 | CA | ILE | C | 406 | 6.974 | 37.184 | −1.841 | 1.00 | 53.53 | C |
| ATOM | 10475 | CB | ILE | C | 406 | 6.885 | 35.685 | −1.508 | 1.00 | 73.34 | C |
| ATOM | 10476 | CG2 | ILE | C | 406 | 8.278 | 35.120 | −1.223 | 1.00 | 60.96 | C |
| ATOM | 10477 | CG1 | ILE | C | 406 | 5.988 | 35.486 | −0.295 | 1.00 | 60.96 | C |
| ATOM | 10478 | CD1 | ILE | C | 406 | 5.911 | 34.043 | 0.179 | 1.00 | 60.96 | C |
| ATOM | 10479 | C | ILE | C | 406 | 7.750 | 37.339 | −3.133 | 1.00 | 60.40 | C |
| ATOM | 10480 | O | ILE | C | 406 | 7.184 | 37.215 | −4.217 | 1.00 | 55.50 | C |
| ATOM | 10481 | N | LYS | C | 407 | 9.041 | 37.624 | −3.016 | 1.00 | 74.12 | C |
| ATOM | 10482 | CA | LYS | C | 407 | 9.906 | 37.762 | −4.179 | 1.00 | 69.72 | C |
| ATOM | 10483 | CB | LYS | C | 407 | 10.156 | 39.234 | −4.521 | 1.00 | 122.47 | C |
| ATOM | 10484 | CG | LYS | C | 407 | 8.921 | 40.017 | −4.920 | 1.00 | 108.57 | C |
| ATOM | 10485 | CD | LYS | C | 407 | 9.301 | 41.392 | −5.448 | 1.00 | 108.57 | C |
| ATOM | 10486 | CE | LYS | C | 407 | 8.077 | 42.259 | −5.707 | 1.00 | 108.57 | C |
| ATOM | 10487 | NZ | LYS | C | 407 | 7.344 | 42.585 | −4.449 | 1.00 | 108.57 | C |
| ATOM | 10488 | C | LYS | C | 407 | 11.213 | 37.114 | −3.785 | 1.00 | 79.03 | C |
| ATOM | 10489 | O | LYS | C | 407 | 11.637 | 37.235 | −2.635 | 1.00 | 77.97 | C |
| ATOM | 10490 | N | LYS | C | 408 | 11.844 | 36.405 | −4.714 | 1.00 | 65.63 | C |
| ATOM | 10491 | CA | LYS | C | 408 | 13.117 | 35.784 | −4.392 | 1.00 | 65.22 | C |
| ATOM | 10492 | CB | LYS | C | 408 | 13.486 | 34.741 | −5.438 | 1.00 | 73.93 | C |
| ATOM | 10493 | CG | LYS | C | 408 | 14.725 | 33.960 | −5.076 | 1.00 | 72.73 | C |
| ATOM | 10494 | CD | LYS | C | 408 | 14.902 | 32.783 | −5.995 | 1.00 | 72.73 | C |
| ATOM | 10495 | CE | LYS | C | 408 | 16.095 | 31.944 | −5.578 | 1.00 | 72.73 | C |
| ATOM | 10496 | NZ | LYS | C | 408 | 16.184 | 30.696 | −6.394 | 1.00 | 72.73 | C |
| ATOM | 10497 | C | LYS | C | 408 | 14.105 | 36.934 | −4.412 | 1.00 | 65.35 | C |
| ATOM | 10498 | O | LYS | C | 408 | 14.403 | 37.462 | −5.469 | 1.00 | 63.92 | C |
| ATOM | 10499 | N | ASP | C | 409 | 14.602 | 37.347 | −3.254 | 1.00 | 101.26 | C |
| ATOM | 10500 | CA | ASP | C | 409 | 15.514 | 38.479 | −3.253 | 1.00 | 101.44 | C |
| ATOM | 10501 | CB | ASP | C | 409 | 15.805 | 38.956 | −1.814 | 1.00 | 74.41 | C |
| ATOM | 10502 | CG | ASP | C | 409 | 16.125 | 37.827 | −0.864 | 1.00 | 67.23 | C |
| ATOM | 10503 | OD1 | ASP | C | 409 | 16.943 | 36.961 | −1.227 | 1.00 | 67.23 | C |
| ATOM | 10504 | OD2 | ASP | C | 409 | 15.569 | 37.823 | 0.251 | 1.00 | 67.23 | C |
| ATOM | 10505 | C | ASP | C | 409 | 16.808 | 38.241 | −4.033 | 1.00 | 101.35 | C |
| ATOM | 10506 | O | ASP | C | 409 | 17.151 | 37.102 | −4.358 | 1.00 | 105.08 | C |
| ATOM | 10507 | N | LYS | C | 410 | 17.484 | 39.350 | −4.351 | 1.00 | 105.37 | C |
| ATOM | 10508 | CA | LYS | C | 410 | 18.742 | 39.393 | −5.103 | 1.00 | 111.81 | C |
| ATOM | 10509 | CB | LYS | C | 410 | 19.609 | 40.545 | −4.590 | 1.00 | 175.34 | C |
| ATOM | 10510 | CG | LYS | C | 410 | 20.148 | 40.353 | −3.168 | 1.00 | 114.79 | C |
| ATOM | 10511 | CD | LYS | C | 410 | 19.065 | 40.492 | −2.094 | 1.00 | 114.79 | C |
| ATOM | 10512 | CE | LYS | C | 410 | 19.635 | 40.258 | −0.694 | 1.00 | 114.79 | C |
| ATOM | 10513 | NZ | LYS | C | 410 | 18.601 | 40.384 | 0.370 | 1.00 | 114.79 | C |
| ATOM | 10514 | C | LYS | C | 410 | 19.562 | 38.111 | −5.073 | 1.00 | 110.63 | C |
| ATOM | 10515 | O | LYS | C | 410 | 19.470 | 37.329 | −4.133 | 1.00 | 110.16 | C |
| ATOM | 10516 | N | ASP | C | 411 | 20.382 | 37.924 | −6.103 | 1.00 | 67.92 | C |
| ATOM | 10517 | CA | ASP | C | 411 | 21.238 | 36.745 | −6.240 | 1.00 | 65.43 | C |
| ATOM | 10518 | CB | ASP | C | 411 | 22.586 | 37.127 | −6.849 | 1.00 | 199.97 | C |
| ATOM | 10519 | CG | ASP | C | 411 | 22.573 | 37.071 | −8.361 | 1.00 | 199.97 | C |
| ATOM | 10520 | OD1 | ASP | C | 411 | 22.139 | 36.041 | −8.910 | 1.00 | 199.97 | C |
| ATOM | 10521 | OD2 | ASP | C | 411 | 23.003 | 38.041 | −9.008 | 1.00 | 199.97 | C |
| ATOM | 10522 | C | ASP | C | 411 | 21.479 | 35.958 | −4.964 | 1.00 | 67.04 | C |
| ATOM | 10523 | O | ASP | C | 411 | 22.505 | 36.119 | −4.307 | 1.00 | 71.22 | C |
| ATOM | 10524 | N | ALA | C | 412 | 20.526 | 35.090 | −4.637 | 1.00 | 80.03 | C |
| ATOM | 10525 | CA | ALA | C | 412 | 20.592 | 34.252 | −3.446 | 1.00 | 93.05 | C |
| ATOM | 10526 | CB | ALA | C | 412 | 20.458 | 35.105 | −2.197 | 1.00 | 75.55 | C |
| ATOM | 10527 | C | ALA | C | 412 | 19.482 | 33.206 | −3.481 | 1.00 | 87.75 | C |
| ATOM | 10528 | O | ALA | C | 412 | 18.608 | 33.240 | −4.348 | 1.00 | 82.80 | C |
| ATOM | 10529 | N | ASN | C | 413 | 19.517 | 32.282 | −2.527 | 1.00 | 80.56 | C |
| ATOM | 10530 | CA | ASN | C | 413 | 18.521 | 31.220 | −2.460 | 1.00 | 73.37 | C |
| ATOM | 10531 | CB | ASN | C | 413 | 19.137 | 29.947 | −1.884 | 1.00 | 63.43 | C |
| ATOM | 10532 | CG | ASN | C | 413 | 20.266 | 29.412 | −2.734 | 1.00 | 63.43 | C |
| ATOM | 10533 | OD1 | ASN | C | 413 | 20.181 | 29.393 | −3.965 | 1.00 | 63.43 | C |
| ATOM | 10534 | ND2 | ASN | C | 413 | 21.328 | 28.958 | −2.081 | 1.00 | 63.43 | C |
| ATOM | 10535 | C | ASN | C | 413 | 17.353 | 31.621 | −1.594 | 1.00 | 77.95 | C |
| ATOM | 10536 | O | ASN | C | 413 | 16.298 | 30.997 | −1.631 | 1.00 | 77.64 | C |
| ATOM | 10537 | N | THR | C | 414 | 17.553 | 32.680 | −0.827 | 1.00 | 53.90 | C |
| ATOM | 10538 | CA | THR | C | 414 | 16.557 | 33.166 | 0.104 | 1.00 | 56.94 | C |
| ATOM | 10539 | CB | THR | C | 414 | 17.269 | 33.962 | 1.199 | 1.00 | 102.49 | C |
| ATOM | 10540 | OG1 | THR | C | 414 | 16.306 | 34.456 | 2.119 | 1.00 | 72.98 | C |
| ATOM | 10541 | CG2 | THR | C | 414 | 18.055 | 35.114 | 0.613 | 1.00 | 72.98 | C |
| ATOM | 10542 | C | THR | C | 414 | 15.415 | 33.981 | −0.519 | 1.00 | 56.40 | C |
| ATOM | 10543 | O | THR | C | 414 | 15.505 | 34.397 | −1.672 | 1.00 | 57.47 | C |
| ATOM | 10544 | N | TYR | C | 415 | 14.332 | 34.179 | 0.236 | 1.00 | 44.77 | C |
| ATOM | 10545 | CA | TYR | C | 415 | 13.155 | 34.933 | −0.231 | 1.00 | 45.80 | C |
| ATOM | 10546 | CB | TYR | C | 415 | 11.942 | 34.020 | −0.414 | 1.00 | 80.90 | C |
| ATOM | 10547 | CG | TYR | C | 415 | 12.082 | 33.004 | −1.508 | 1.00 | 47.84 | C |
| ATOM | 10548 | CD1 | TYR | C | 415 | 12.977 | 31.944 | −1.390 | 1.00 | 47.84 | C |
| ATOM | 10549 | CE1 | TYR | C | 415 | 13.126 | 31.011 | −2.408 | 1.00 | 47.84 | C |
| ATOM | 10550 | CD2 | TYR | C | 415 | 11.332 | 33.108 | −2.673 | 1.00 | 47.84 | C |
| ATOM | 10551 | CE2 | TYR | C | 415 | 11.469 | 32.181 | −3.703 | 1.00 | 47.84 | C |
| ATOM | 10552 | CZ | TYR | C | 415 | 12.370 | 31.132 | −3.564 | 1.00 | 47.84 | C |
| ATOM | 10553 | OH | TYR | C | 415 | 12.520 | 30.193 | −4.567 | 1.00 | 47.84 | C |
| ATOM | 10554 | C | TYR | C | 415 | 12.753 | 36.015 | 0.764 | 1.00 | 53.36 | C |
| ATOM | 10555 | O | TYR | C | 415 | 13.160 | 35.977 | 1.931 | 1.00 | 48.01 | C |
| ATOM | 10556 | N | SER | C | 416 | 11.936 | 36.965 | 0.307 | 1.00 | 44.13 | C |
| ATOM | 10557 | CA | SER | C | 416 | 11.481 | 38.053 | 1.168 | 1.00 | 42.08 | C |
| ATOM | 10558 | CB | SER | C | 416 | 12.246 | 39.346 | 0.867 | 1.00 | 76.72 | C |
| ATOM | 10559 | OG | SER | C | 416 | 13.574 | 39.282 | 1.369 | 1.00 | 53.10 | C |
| ATOM | 10560 | C | SER | C | 416 | 9.993 | 38.266 | 0.984 | 1.00 | 48.27 | C |
| ATOM | 10561 | O | SER | C | 416 | 9.445 | 37.990 | −0.084 | 1.00 | 46.64 | C |
| ATOM | 10562 | N | LEU | C | 417 | 9.350 | 38.768 | 2.034 | 1.00 | 46.65 | C |
| ATOM | 10563 | CA | LEU | C | 417 | 7.914 | 38.985 | 2.027 | 1.00 | 45.55 | C |
| ATOM | 10564 | CB | LEU | C | 417 | 7.236 | 37.845 | 2.793 | 1.00 | 100.23 | C |
| ATOM | 10565 | CG | LEU | C | 417 | 5.890 | 38.189 | 3.435 | 1.00 | 58.34 | C |
| ATOM | 10566 | CD1 | LEU | C | 417 | 4.792 | 38.190 | 2.386 | 1.00 | 58.34 | C |
| ATOM | 10567 | CD2 | LEU | C | 417 | 5.582 | 37.200 | 4.506 | 1.00 | 58.34 | C |
| ATOM | 10568 | C | LEU | C | 417 | 7.496 | 40.313 | 2.644 | 1.00 | 57.06 | C |
| ATOM | 10569 | O | LEU | C | 417 | 8.167 | 40.842 | 3.521 | 1.00 | 52.16 | C |
| ATOM | 10570 | N | THR | C | 418 | 6.363 | 40.832 | 2.191 | 1.00 | 58.77 | C |
| ATOM | 10571 | CA | THR | C | 418 | 5.826 | 42.079 | 2.712 | 1.00 | 51.83 | C |
| ATOM | 10572 | CB | THR | C | 418 | 6.117 | 43.268 | 1.767 | 1.00 | 84.49 | C |
| ATOM | 10573 | OG1 | THR | C | 418 | 7.505 | 43.600 | 1.826 | 1.00 | 52.90 | C |
| ATOM | 10574 | CG2 | THR | C | 418 | 5.308 | 44.486 | 2.169 | 1.00 | 52.90 | C |
| ATOM | 10575 | C | THR | C | 418 | 4.322 | 41.930 | 2.824 | 1.00 | 67.02 | C |
| ATOM | 10576 | O | THR | C | 418 | 3.640 | 41.768 | 1.816 | 1.00 | 60.49 | C |
| ATOM | 10577 | N | ASP | C | 419 | 3.799 | 41.985 | 4.041 | 1.00 | 44.56 | C |
| ATOM | 10578 | CA | ASP | C | 419 | 2.364 | 41.852 | 4.224 | 1.00 | 49.67 | C |
| ATOM | 10579 | CB | ASP | C | 419 | 2.050 | 40.651 | 5.120 | 1.00 | 124.28 | C |
| ATOM | 10580 | CG | ASP | C | 419 | 0.589 | 40.251 | 5.059 | 1.00 | 101.87 | C |
| ATOM | 10581 | OD1 | ASP | C | 419 | 0.061 | 40.125 | 3.935 | 1.00 | 101.87 | C |
| ATOM | 10582 | OD2 | ASP | C | 419 | −0.032 | 40.058 | 6.125 | 1.00 | 101.87 | C |
| ATOM | 10583 | C | ASP | C | 419 | 1.783 | 43.125 | 4.820 | 1.00 | 53.76 | C |
| ATOM | 10584 | O | ASP | C | 419 | 2.221 | 43.580 | 5.872 | 1.00 | 48.65 | C |
| ATOM | 10585 | N | LEU | C | 420 | 0.787 | 43.692 | 4.145 | 1.00 | 74.57 | C |
| ATOM | 10586 | CA | LEU | C | 420 | 0.153 | 44.930 | 4.590 | 1.00 | 79.26 | C |
| ATOM | 10587 | CB | LEU | C | 420 | 0.031 | 45.881 | 3.401 | 1.00 | 130.74 | C |
| ATOM | 10588 | CG | LEU | C | 420 | −0.140 | 47.366 | 3.702 | 1.00 | 89.69 | C |
| ATOM | 10589 | CD1 | LEU | C | 420 | 1.117 | 47.883 | 4.391 | 1.00 | 89.69 | C |
| ATOM | 10590 | CD2 | LEU | C | 420 | −0.394 | 48.129 | 2.403 | 1.00 | 89.69 | C |
| ATOM | 10591 | C | LEU | C | 420 | −1.228 | 44.684 | 5.204 | 1.00 | 81.83 | C |
| ATOM | 10592 | O | LEU | C | 420 | −1.878 | 43.691 | 4.888 | 1.00 | 81.15 | C |
| ATOM | 10593 | N | VAL | C | 421 | −1.671 | 45.585 | 6.080 | 1.00 | 65.55 | C |
| ATOM | 10594 | CA | VAL | C | 421 | −2.979 | 45.460 | 6.728 | 1.00 | 64.06 | C |
| ATOM | 10595 | CB | VAL | C | 421 | −2.858 | 44.742 | 8.087 | 1.00 | 109.24 | C |
| ATOM | 10596 | CG1 | VAL | C | 421 | −4.186 | 44.768 | 8.830 | 1.00 | 64.67 | C |
| ATOM | 10597 | CG2 | VAL | C | 421 | −2.444 | 43.325 | 7.857 | 1.00 | 64.67 | C |
| ATOM | 10598 | C | VAL | C | 421 | −3.657 | 46.808 | 6.950 | 1.00 | 66.32 | C |
| ATOM | 10599 | O | VAL | C | 421 | −3.030 | 47.764 | 7.393 | 1.00 | 61.92 | C |
| ATOM | 10600 | N | ARG | C | 422 | −4.945 | 46.871 | 6.649 | 1.00 | 83.02 | C |
| ATOM | 10601 | CA | ARG | C | 422 | −5.724 | 48.095 | 6.812 | 1.00 | 85.15 | C |
| ATOM | 10602 | CB | ARG | C | 422 | −6.944 | 48.032 | 5.893 | 1.00 | 159.06 | C |
| ATOM | 10603 | CG | ARG | C | 422 | −7.094 | 49.197 | 4.938 | 1.00 | 119.65 | C |
| ATOM | 10604 | CD | ARG | C | 422 | −7.682 | 50.419 | 5.623 | 1.00 | 119.65 | C |
| ATOM | 10605 | NE | ARG | C | 422 | −7.960 | 51.488 | 4.661 | 1.00 | 119.65 | C |
| ATOM | 10606 | CZ | ARG | C | 422 | −8.587 | 52.626 | 4.956 | 1.00 | 119.65 | C |
| ATOM | 10607 | NH1 | ARG | C | 422 | −9.010 | 52.855 | 6.193 | 1.00 | 119.65 | C |
| ATOM | 10608 | NH2 | ARG | C | 422 | −8.793 | 53.537 | 4.013 | 1.00 | 119.65 | C |
| ATOM | 10609 | C | ARG | C | 422 | −6.177 | 48.233 | 8.267 | 1.00 | 82.38 | C |
| ATOM | 10610 | O | ARG | C | 422 | −7.130 | 47.582 | 8.682 | 1.00 | 73.05 | C |
| ATOM | 10611 | N | THR | C | 423 | −5.506 | 49.076 | 9.043 | 1.00 | 113.45 | C |
| ATOM | 10612 | CA | THR | C | 423 | −5.866 | 49.255 | 10.449 | 1.00 | 123.10 | C |
| ATOM | 10613 | CB | THR | C | 423 | −4.765 | 50.026 | 11.226 | 1.00 | 171.80 | C |
| ATOM | 10614 | OG1 | THR | C | 423 | −3.515 | 49.346 | 11.089 | 1.00 | 127.59 | C |
| ATOM | 10615 | CG2 | THR | C | 423 | −5.108 | 50.115 | 12.713 | 1.00 | 127.59 | C |
| ATOM | 10616 | C | THR | C | 423 | −7.178 | 50.010 | 10.618 | 1.00 | 121.32 | C |
| ATOM | 10617 | O | THR | C | 423 | −7.192 | 51.109 | 11.162 | 1.00 | 119.81 | C |
| ATOM | 10618 | N | THR | C | 424 | −8.282 | 49.429 | 10.157 | 1.00 | 164.75 | C |
| ATOM | 10619 | CA | THR | C | 424 | −9.579 | 50.085 | 10.295 | 1.00 | 164.75 | C |
| ATOM | 10620 | CB | THR | C | 424 | −10.695 | 49.282 | 9.603 | 1.00 | 186.34 | C |
| ATOM | 10621 | OG1 | THR | C | 424 | −10.362 | 49.105 | 8.221 | 1.00 | 132.15 | C |
| ATOM | 10622 | CG2 | THR | C | 424 | −12.028 | 50.019 | 9.705 | 1.00 | 132.15 | C |
| ATOM | 10623 | C | THR | C | 424 | −9.903 | 50.219 | 11.778 | 1.00 | 164.75 | C |
| ATOM | 10624 | O | THR | C | 424 | −9.394 | 49.457 | 12.601 | 1.00 | 164.75 | C |
| ATOM | 10625 | N | SER | C | 425 | −10.748 | 51.186 | 12.116 | 1.00 | 153.38 | C |
| ATOM | 10626 | CA | SER | C | 425 | −11.114 | 51.422 | 13.507 | 1.00 | 153.38 | C |
| ATOM | 10627 | CB | SER | C | 425 | −11.758 | 52.800 | 13.647 | 1.00 | 183.67 | C |
| ATOM | 10628 | OG | SER | C | 425 | −11.996 | 53.102 | 15.009 | 1.00 | 79.61 | C |
| ATOM | 10629 | C | SER | C | 425 | −12.043 | 50.361 | 14.100 | 1.00 | 153.38 | C |
| ATOM | 10630 | O | SER | C | 425 | −12.408 | 50.437 | 15.273 | 1.00 | 153.38 | C |
| ATOM | 10631 | N | LYS | C | 426 | −12.422 | 49.374 | 13.294 | 1.00 | 176.45 | C |
| ATOM | 10632 | CA | LYS | C | 426 | −13.308 | 48.305 | 13.754 | 1.00 | 176.45 | C |
| ATOM | 10633 | CB | LYS | C | 426 | −13.540 | 47.296 | 12.631 | 1.00 | 182.96 | C |
| ATOM | 10634 | CG | LYS | C | 426 | −13.982 | 47.910 | 11.321 | 1.00 | 127.26 | C |
| ATOM | 10635 | CD | LYS | C | 426 | −14.001 | 46.861 | 10.229 | 1.00 | 127.26 | C |
| ATOM | 10636 | CE | LYS | C | 426 | −14.303 | 47.472 | 8.876 | 1.00 | 127.26 | C |
| ATOM | 10637 | NZ | LYS | C | 426 | −14.282 | 46.439 | 7.803 | 1.00 | 127.26 | C |
| ATOM | 10638 | C | LYS | C | 426 | −12.720 | 47.575 | 14.959 | 1.00 | 176.45 | C |
| ATOM | 10639 | O | LYS | C | 426 | −13.438 | 47.215 | 15.891 | 1.00 | 176.45 | C |
| ATOM | 10640 | N | MET | C | 427 | −11.408 | 47.359 | 14.927 | 1.00 | 166.25 | C |
| ATOM | 10641 | CA | MET | C | 427 | −10.700 | 46.663 | 15.999 | 1.00 | 166.25 | C |
| ATOM | 10642 | CB | MET | C | 427 | −9.256 | 46.386 | 15.574 | 1.00 | 146.92 | C |
| ATOM | 10643 | CG | MET | C | 427 | −8.510 | 47.614 | 15.080 | 1.00 | 146.92 | C |
| ATOM | 10644 | SD | MET | C | 427 | −6.842 | 47.217 | 14.540 | 1.00 | 146.92 | C |
| ATOM | 10645 | CE | MET | C | 427 | −7.159 | 46.496 | 12.927 | 1.00 | 146.92 | C |
| ATOM | 10646 | C | MET | C | 427 | −10.704 | 47.423 | 17.320 | 1.00 | 166.25 | C |
| ATOM | 10647 | O | MET | C | 427 | −11.519 | 48.320 | 17.530 | 1.00 | 166.25 | C |
| ATOM | 10648 | N | SER | C | 428 | −9.786 | 47.054 | 18.208 | 1.00 | 125.19 | C |
| ATOM | 10649 | CA | SER | C | 428 | −9.692 | 47.694 | 19.512 | 1.00 | 126.84 | C |
| ATOM | 10650 | CB | SER | C | 428 | −10.922 | 47.343 | 20.350 | 1.00 | 119.10 | C |
| ATOM | 10651 | OG | SER | C | 428 | −10.991 | 45.947 | 20.581 | 1.00 | 115.81 | C |
| ATOM | 10652 | C | SER | C | 428 | −8.433 | 47.294 | 20.280 | 1.00 | 122.79 | C |
| ATOM | 10653 | O | SER | C | 428 | −8.448 | 47.252 | 21.509 | 1.00 | 122.67 | C |
| ATOM | 10654 | N | SER | C | 429 | −7.348 | 47.002 | 19.568 | 1.00 | 137.25 | C |
| ATOM | 10655 | CA | SER | C | 429 | −6.100 | 46.611 | 20.223 | 1.00 | 135.92 | C |
| ATOM | 10656 | CB | SER | C | 429 | −6.165 | 45.148 | 20.679 | 1.00 | 117.80 | C |
| ATOM | 10657 | OG | SER | C | 429 | −7.156 | 44.949 | 21.667 | 1.00 | 117.80 | C |
| ATOM | 10658 | C | SER | C | 429 | −4.880 | 46.776 | 19.330 | 1.00 | 134.30 | C |
| ATOM | 10659 | O | SER | C | 429 | −3.751 | 46.650 | 19.793 | 1.00 | 123.00 | C |
| ATOM | 10660 | N | GLN | C | 430 | −5.105 | 47.064 | 18.056 | 1.00 | 69.91 | C |
| ATOM | 10661 | CA | GLN | C | 430 | −4.012 | 47.201 | 17.096 | 1.00 | 69.91 | C |
| ATOM | 10662 | CB | GLN | C | 430 | −2.987 | 48.231 | 17.553 | 1.00 | 178.23 | C |
| ATOM | 10663 | CG | GLN | C | 430 | −1.887 | 48.424 | 16.534 | 1.00 | 107.22 | C |
| ATOM | 10664 | CD | GLN | C | 430 | −0.832 | 49.402 | 16.986 | 1.00 | 107.22 | C |
| ATOM | 10665 | OE1 | GLN | C | 430 | −0.064 | 49.125 | 17.906 | 1.00 | 107.22 | C |
| ATOM | 10666 | NE2 | GLN | C | 430 | −0.789 | 50.561 | 16.342 | 1.00 | 107.22 | C |
| ATOM | 10667 | C | GLN | C | 430 | −3.336 | 45.841 | 16.951 | 1.00 | 69.91 | C |
| ATOM | 10668 | O | GLN | C | 430 | −2.661 | 45.362 | 17.861 | 1.00 | 69.91 | C |
| ATOM | 10669 | N | PRO | C | 431 | −3.491 | 45.210 | 15.784 | 1.00 | 75.11 | C |
| ATOM | 10670 | CD | PRO | C | 431 | −3.948 | 45.770 | 14.502 | 1.00 | 58.79 | C |
| ATOM | 10671 | CA | PRO | C | 431 | −2.886 | 43.901 | 15.579 | 1.00 | 75.10 | C |
| ATOM | 10672 | CB | PRO | C | 431 | −3.300 | 43.558 | 14.154 | 1.00 | 157.75 | C |
| ATOM | 10673 | CG | PRO | C | 431 | −3.260 | 44.876 | 13.492 | 1.00 | 128.31 | C |
| ATOM | 10674 | C | PRO | C | 431 | −1.387 | 43.872 | 15.765 | 1.00 | 71.72 | C |
| ATOM | 10675 | O | PRO | C | 431 | −0.706 | 44.897 | 15.701 | 1.00 | 71.60 | C |
| ATOM | 10676 | N | VAL | C | 432 | −0.897 | 42.670 | 16.020 | 1.00 | 62.25 | C |
| ATOM | 10677 | CA | VAL | C | 432 | 0.512 | 42.409 | 16.181 | 1.00 | 64.63 | C |
| ATOM | 10678 | CB | VAL | C | 432 | 0.797 | 41.829 | 17.564 | 1.00 | 86.55 | C |
| ATOM | 10679 | CG1 | VAL | C | 432 | 0.053 | 40.537 | 17.755 | 1.00 | 58.63 | C |
| ATOM | 10680 | CG2 | VAL | C | 432 | 2.251 | 41.607 | 17.719 | 1.00 | 58.63 | C |
| ATOM | 10681 | C | VAL | C | 432 | 0.761 | 41.372 | 15.087 | 1.00 | 62.63 | C |
| ATOM | 10682 | O | VAL | C | 432 | −0.147 | 40.622 | 14.723 | 1.00 | 52.99 | C |
| ATOM | 10683 | N | CYS | C | 433 | 1.964 | 41.327 | 14.536 | 1.00 | 66.85 | C |
| ATOM | 10684 | CA | CYS | C | 433 | 2.226 | 40.376 | 13.470 | 1.00 | 62.73 | C |
| ATOM | 10685 | C | CYS | C | 433 | 2.825 | 39.062 | 13.974 | 1.00 | 60.37 | C |
| ATOM | 10686 | O | CYS | C | 433 | 3.862 | 39.067 | 14.639 | 1.00 | 61.11 | C |
| ATOM | 10687 | CB | CYS | C | 433 | 3.154 | 41.012 | 12.426 | 1.00 | 128.59 | C |
| ATOM | 10688 | SG | CYS | C | 433 | 3.418 | 40.001 | 10.932 | 1.00 | 75.70 | C |
| ATOM | 10689 | N | VAL | C | 434 | 2.162 | 37.941 | 13.673 | 1.00 | 58.06 | C |
| ATOM | 10690 | CA | VAL | C | 434 | 2.670 | 36.626 | 14.056 | 1.00 | 54.05 | C |
| ATOM | 10691 | CB | VAL | C | 434 | 1.590 | 35.745 | 14.685 | 1.00 | 64.63 | C |
| ATOM | 10692 | CG1 | VAL | C | 434 | 2.236 | 34.486 | 15.247 | 1.00 | 46.41 | C |
| ATOM | 10693 | CG2 | VAL | C | 434 | 0.869 | 36.504 | 15.781 | 1.00 | 46.41 | C |
| ATOM | 10694 | C | VAL | C | 434 | 3.182 | 35.927 | 12.798 | 1.00 | 64.45 | C |
| ATOM | 10695 | O | VAL | C | 434 | 2.415 | 35.650 | 11.881 | 1.00 | 55.51 | C |
| ATOM | 10696 | N | VAL | C | 435 | 4.476 | 35.635 | 12.768 | 1.00 | 49.92 | C |
| ATOM | 10697 | CA | VAL | C | 435 | 5.107 | 35.012 | 11.615 | 1.00 | 41.66 | C |
| ATOM | 10698 | CB | VAL | C | 435 | 6.441 | 35.723 | 11.305 | 1.00 | 56.35 | C |
| ATOM | 10699 | CG1 | VAL | C | 435 | 7.230 | 34.960 | 10.259 | 1.00 | 35.68 | C |
| ATOM | 10700 | CG2 | VAL | C | 435 | 6.160 | 37.128 | 10.840 | 1.00 | 35.68 | C |
| ATOM | 10701 | C | VAL | C | 435 | 5.375 | 33.518 | 11.770 | 1.00 | 49.58 | C |
| ATOM | 10702 | O | VAL | C | 435 | 6.077 | 33.098 | 12.684 | 1.00 | 50.17 | C |
| ATOM | 10703 | N | PHE | C | 436 | 4.827 | 32.719 | 10.861 | 1.00 | 59.43 | C |
| ATOM | 10704 | CA | PHE | C | 436 | 5.026 | 31.281 | 10.899 | 1.00 | 55.63 | C |
| ATOM | 10705 | CB | PHE | C | 436 | 3.704 | 30.551 | 10.684 | 1.00 | 62.47 | C |
| ATOM | 10706 | CG | PHE | C | 436 | 2.697 | 30.794 | 11.763 | 1.00 | 37.24 | C |
| ATOM | 10707 | CD1 | PHE | C | 436 | 1.868 | 31.905 | 11.726 | 1.00 | 37.24 | C |
| ATOM | 10708 | CD2 | PHE | C | 436 | 2.591 | 29.917 | 12.841 | 1.00 | 37.24 | C |
| ATOM | 10709 | CE1 | PHE | C | 436 | 0.941 | 32.141 | 12.749 | 1.00 | 37.24 | C |
| ATOM | 10710 | CE2 | PHE | C | 436 | 1.668 | 30.147 | 13.869 | 1.00 | 37.24 | C |
| ATOM | 10711 | CZ | PHE | C | 436 | 0.843 | 31.256 | 13.823 | 1.00 | 37.24 | C |
| ATOM | 10712 | C | PHE | C | 436 | 6.019 | 30.854 | 9.830 | 1.00 | 63.46 | C |
| ATOM | 10713 | O | PHE | C | 436 | 5.792 | 31.067 | 8.639 | 1.00 | 62.84 | C |
| ATOM | 10714 | N | HIS | C | 437 | 7.121 | 30.246 | 10.258 | 1.00 | 41.66 | C |
| ATOM | 10715 | CA | HIS | C | 437 | 8.157 | 29.798 | 9.339 | 1.00 | 42.73 | C |
| ATOM | 10716 | CB | HIS | C | 437 | 9.114 | 30.950 | 9.033 | 1.00 | 72.91 | C |
| ATOM | 10717 | CG | HIS | C | 437 | 10.202 | 30.588 | 8.076 | 1.00 | 49.28 | C |
| ATOM | 10718 | CD2 | HIS | C | 437 | 11.486 | 30.214 | 8.286 | 1.00 | 49.28 | C |
| ATOM | 10719 | ND1 | HIS | C | 437 | 10.006 | 30.536 | 6.714 | 1.00 | 49.28 | C |
| ATOM | 10720 | CE1 | HIS | C | 437 | 11.123 | 30.147 | 6.125 | 1.00 | 49.28 | C |
| ATOM | 10721 | NE2 | HIS | C | 437 | 12.036 | 29.942 | 7.057 | 1.00 | 49.28 | C |
| ATOM | 10722 | C | HIS | C | 437 | 8.926 | 28.643 | 9.978 | 1.00 | 47.04 | C |
| ATOM | 10723 | O | HIS | C | 437 | 8.905 | 28.485 | 11.195 | 1.00 | 49.26 | C |
| ATOM | 10724 | N | ASP | C | 438 | 9.601 | 27.836 | 9.163 | 1.00 | 51.67 | C |
| ATOM | 10725 | CA | ASP | C | 438 | 10.363 | 26.700 | 9.672 | 1.00 | 53.58 | C |
| ATOM | 10726 | CB | ASP | C | 438 | 11.085 | 25.996 | 8.529 | 1.00 | 105.11 | C |
| ATOM | 10727 | CG | ASP | C | 438 | 10.189 | 25.007 | 7.815 | 1.00 | 76.07 | C |
| ATOM | 10728 | OD1 | ASP | C | 438 | 8.948 | 25.171 | 7.903 | 1.00 | 76.07 | C |
| ATOM | 10729 | OD2 | ASP | C | 438 | 10.719 | 24.074 | 7.166 | 1.00 | 76.07 | C |
| ATOM | 10730 | C | ASP | C | 438 | 11.337 | 27.117 | 10.760 | 1.00 | 54.92 | C |
| ATOM | 10731 | O | ASP | C | 438 | 11.405 | 26.481 | 11.804 | 1.00 | 58.48 | C |
| ATOM | 10732 | N | THR | C | 439 | 12.104 | 28.173 | 10.520 | 1.00 | 77.01 | C |
| ATOM | 10733 | CA | THR | C | 439 | 13.015 | 28.682 | 11.537 | 1.00 | 74.36 | C |
| ATOM | 10734 | CB | THR | C | 439 | 14.077 | 29.600 | 10.899 | 1.00 | 64.56 | C |
| ATOM | 10735 | OG1 | THR | C | 439 | 13.445 | 30.738 | 10.299 | 1.00 | 50.37 | C |
| ATOM | 10736 | CG2 | THR | C | 439 | 14.825 | 28.847 | 9.804 | 1.00 | 50.37 | C |
| ATOM | 10737 | C | THR | C | 439 | 12.027 | 29.473 | 12.393 | 1.00 | 80.13 | C |
| ATOM | 10738 | O | THR | C | 439 | 11.072 | 30.020 | 11.859 | 1.00 | 80.47 | C |
| ATOM | 10739 | N | LEU | C | 440 | 12.222 | 29.533 | 13.702 | 1.00 | 70.88 | C |
| ATOM | 10740 | CA | LEU | C | 440 | 11.267 | 30.231 | 14.583 | 1.00 | 72.90 | C |
| ATOM | 10741 | CB | LEU | C | 440 | 11.334 | 31.778 | 14.420 | 1.00 | 68.94 | C |
| ATOM | 10742 | CG | LEU | C | 440 | 11.069 | 32.776 | 13.254 | 1.00 | 48.94 | C |
| ATOM | 10743 | CD1 | LEU | C | 440 | 12.135 | 32.637 | 12.210 | 1.00 | 48.94 | C |
| ATOM | 10744 | CD2 | LEU | C | 440 | 9.691 | 32.622 | 12.635 | 1.00 | 48.94 | C |
| ATOM | 10745 | C | LEU | C | 440 | 9.821 | 29.747 | 14.379 | 1.00 | 82.85 | C |
| ATOM | 10746 | O | LEU | C | 440 | 9.016 | 30.437 | 13.763 | 1.00 | 81.07 | C |
| ATOM | 10747 | N | GLU | C | 441 | 9.498 | 28.561 | 14.905 | 1.00 | 58.36 | C |
| ATOM | 10748 | CA | GLU | C | 441 | 8.156 | 27.964 | 14.779 | 1.00 | 58.36 | C |
| ATOM | 10749 | CB | GLU | C | 441 | 7.886 | 27.021 | 15.949 | 1.00 | 187.94 | C |
| ATOM | 10750 | CG | GLU | C | 441 | 8.797 | 25.809 | 15.975 | 1.00 | 111.30 | C |
| ATOM | 10751 | CD | GLU | C | 441 | 8.400 | 24.805 | 17.043 | 1.00 | 111.30 | C |
| ATOM | 10752 | OE1 | GLU | C | 441 | 8.354 | 25.189 | 18.234 | 1.00 | 111.30 | C |
| ATOM | 10753 | OE2 | GLU | C | 441 | 8.132 | 23.630 | 16.692 | 1.00 | 111.30 | C |
| ATOM | 10754 | C | GLU | C | 441 | 7.015 | 28.975 | 14.665 | 1.00 | 58.36 | C |
| ATOM | 10755 | O | GLU | C | 441 | 6.115 | 28.819 | 13.839 | 1.00 | 58.36 | C |
| ATOM | 10756 | N | ALA | C | 442 | 7.050 | 30.011 | 15.493 | 1.00 | 72.33 | C |
| ATOM | 10757 | CA | ALA | C | 442 | 6.029 | 31.050 | 15.437 | 1.00 | 70.55 | C |
| ATOM | 10758 | CB | ALA | C | 442 | 4.673 | 30.477 | 15.810 | 1.00 | 23.09 | C |
| ATOM | 10759 | C | ALA | C | 442 | 6.397 | 32.192 | 16.371 | 1.00 | 66.54 | C |
| ATOM | 10760 | O | ALA | C | 442 | 6.315 | 32.044 | 17.588 | 1.00 | 63.95 | C |
| ATOM | 10761 | N | GLN | C | 443 | 6.816 | 33.322 | 15.799 | 1.00 | 50.40 | C |
| ATOM | 10762 | CA | GLN | C | 443 | 7.193 | 34.499 | 16.585 | 1.00 | 53.68 | C |
| ATOM | 10763 | CB | GLN | C | 443 | 8.610 | 34.952 | 16.244 | 1.00 | 99.89 | C |
| ATOM | 10764 | CG | GLN | C | 443 | 9.669 | 34.301 | 17.091 | 1.00 | 83.12 | C |
| ATOM | 10765 | CD | GLN | C | 443 | 10.956 | 35.090 | 17.090 | 1.00 | 83.12 | C |
| ATOM | 10766 | OE1 | GLN | C | 443 | 10.952 | 36.300 | 17.332 | 1.00 | 83.12 | C |
| ATOM | 10767 | NE2 | GLN | C | 443 | 12.073 | 34.412 | 16.823 | 1.00 | 83.12 | C |
| ATOM | 10768 | C | GLN | C | 443 | 6.241 | 35.677 | 16.421 | 1.00 | 55.85 | C |
| ATOM | 10769 | O | GLN | C | 443 | 5.629 | 35.860 | 15.374 | 1.00 | 56.91 | C |
| ATOM | 10770 | N | VAL | C | 444 | 6.120 | 36.473 | 17.475 | 1.00 | 68.96 | C |
| ATOM | 10771 | CA | VAL | C | 444 | 5.244 | 37.635 | 17.456 | 1.00 | 66.11 | C |
| ATOM | 10772 | CB | VAL | C | 444 | 4.411 | 37.715 | 18.754 | 1.00 | 63.75 | C |
| ATOM | 10773 | CG1 | VAL | C | 444 | 3.261 | 38.670 | 18.568 | 1.00 | 56.05 | C |
| ATOM | 10774 | CG2 | VAL | C | 444 | 3.893 | 36.340 | 19.127 | 1.00 | 56.05 | C |
| ATOM | 10775 | C | VAL | C | 444 | 6.085 | 38.905 | 17.307 | 1.00 | 69.93 | C |
| ATOM | 10776 | O | VAL | C | 444 | 7.172 | 39.011 | 17.876 | 1.00 | 72.19 | C |
| ATOM | 10777 | N | TYR | C | 445 | 5.590 | 39.857 | 16.522 | 1.00 | 48.49 | C |
| ATOM | 10778 | CA | TYR | C | 445 | 6.291 | 41.117 | 16.286 | 1.00 | 47.56 | C |
| ATOM | 10779 | CB | TYR | C | 445 | 6.939 | 41.097 | 14.914 | 1.00 | 78.24 | C |
| ATOM | 10780 | CG | TYR | C | 445 | 7.928 | 39.976 | 14.758 | 1.00 | 50.66 | C |
| ATOM | 10781 | CD1 | TYR | C | 445 | 7.540 | 38.736 | 14.256 | 1.00 | 50.66 | C |
| ATOM | 10782 | CE1 | TYR | C | 445 | 8.459 | 37.699 | 14.106 | 1.00 | 50.66 | C |
| ATOM | 10783 | CD2 | TYR | C | 445 | 9.258 | 40.150 | 15.112 | 1.00 | 50.66 | C |
| ATOM | 10784 | CE2 | TYR | C | 445 | 10.181 | 39.122 | 14.968 | 1.00 | 50.66 | C |
| ATOM | 10785 | CZ | TYR | C | 445 | 9.780 | 37.897 | 14.463 | 1.00 | 50.66 | C |
| ATOM | 10786 | OH | TYR | C | 445 | 10.711 | 36.882 | 14.302 | 1.00 | 50.66 | C |
| ATOM | 10787 | C | TYR | C | 445 | 5.314 | 42.280 | 16.396 | 1.00 | 49.02 | C |
| ATOM | 10788 | O | TYR | C | 445 | 4.195 | 42.210 | 15.884 | 1.00 | 51.88 | C |
| ATOM | 10789 | N | THR | C | 446 | 5.745 | 43.359 | 17.048 | 1.00 | 49.85 | C |
| ATOM | 10790 | CA | THR | C | 446 | 4.871 | 44.512 | 17.266 | 1.00 | 56.83 | C |
| ATOM | 10791 | CB | THR | C | 446 | 4.700 | 44.750 | 18.767 | 1.00 | 73.69 | C |
| ATOM | 10792 | OG1 | THR | C | 446 | 5.994 | 44.939 | 19.355 | 1.00 | 61.84 | C |
| ATOM | 10793 | CG2 | THR | C | 446 | 4.031 | 43.560 | 19.424 | 1.00 | 61.84 | C |
| ATOM | 10794 | C | THR | C | 446 | 5.254 | 45.860 | 16.652 | 1.00 | 66.63 | C |
| ATOM | 10795 | O | THR | C | 446 | 5.980 | 45.943 | 15.661 | 1.00 | 70.30 | C |
| ATOM | 10796 | N | CYS | C | 447 | 4.713 | 46.898 | 17.292 | 1.00 | 87.16 | C |
| ATOM | 10797 | CA | CYS | C | 447 | 4.879 | 48.328 | 16.997 | 1.00 | 88.14 | C |
| ATOM | 10798 | C | CYS | C | 447 | 4.380 | 48.932 | 15.681 | 1.00 | 88.17 | C |
| ATOM | 10799 | O | CYS | C | 447 | 4.495 | 48.346 | 14.604 | 1.00 | 85.03 | C |
| ATOM | 10800 | CB | CYS | C | 447 | 6.333 | 48.771 | 17.247 | 1.00 | 135.66 | C |
| ATOM | 10801 | SG | CYS | C | 447 | 7.637 | 47.967 | 16.267 | 1.00 | 121.19 | C |
| ATOM | 10802 | N | SER | C | 448 | 3.811 | 50.129 | 15.813 | 1.00 | 200.84 | C |
| ATOM | 10803 | CA | SER | C | 448 | 3.271 | 50.896 | 14.696 | 1.00 | 200.84 | C |
| ATOM | 10804 | CB | SER | C | 448 | 1.751 | 51.002 | 14.812 | 1.00 | 200.84 | C |
| ATOM | 10805 | OG | SER | C | 448 | 1.155 | 49.718 | 14.819 | 1.00 | 200.84 | C |
| ATOM | 10806 | C | SER | C | 448 | 3.890 | 52.294 | 14.702 | 1.00 | 200.84 | C |
| ATOM | 10807 | O | SER | C | 448 | 4.988 | 52.481 | 15.226 | 1.00 | 200.84 | C |
| ATOM | 10808 | N | GLU | C | 449 | 3.179 | 53.275 | 14.145 | 1.00 | 122.03 | C |
| ATOM | 10809 | CA | GLU | C | 449 | 3.692 | 54.640 | 14.057 | 1.00 | 122.03 | C |
| ATOM | 10810 | CB | GLU | C | 449 | 4.200 | 55.129 | 15.416 | 1.00 | 117.72 | C |
| ATOM | 10811 | CG | GLU | C | 449 | 3.117 | 55.617 | 16.353 | 1.00 | 117.72 | C |
| ATOM | 10812 | CD | GLU | C | 449 | 1.941 | 54.668 | 16.419 | 1.00 | 117.72 | C |
| ATOM | 10813 | OE1 | GLU | C | 449 | 2.160 | 53.475 | 16.708 | 1.00 | 117.72 | C |
| ATOM | 10814 | OE2 | GLU | C | 449 | 0.797 | 55.113 | 16.181 | 1.00 | 117.72 | C |
| ATOM | 10815 | C | GLU | C | 449 | 4.847 | 54.596 | 13.075 | 1.00 | 122.03 | C |
| ATOM | 10816 | O | GLU | C | 449 | 4.989 | 55.457 | 12.212 | 1.00 | 122.03 | C |
| ATOM | 10817 | N | GLY | C | 450 | 5.661 | 53.561 | 13.221 | 1.00 | 62.64 | C |
| ATOM | 10818 | CA | GLY | C | 450 | 6.817 | 53.348 | 12.376 | 1.00 | 62.64 | C |
| ATOM | 10819 | C | GLY | C | 450 | 7.845 | 52.665 | 13.247 | 1.00 | 62.64 | C |
| ATOM | 10820 | O | GLY | C | 450 | 9.013 | 52.534 | 12.877 | 1.00 | 62.64 | C |
| ATOM | 10821 | N | CYS | C | 451 | 7.388 | 52.225 | 14.416 | 1.00 | 185.56 | C |
| ATOM | 10822 | CA | CYS | C | 451 | 8.240 | 51.571 | 15.395 | 1.00 | 185.56 | C |
| ATOM | 10823 | CB | CYS | C | 451 | 8.862 | 50.311 | 14.821 | 1.00 | 190.59 | C |
| ATOM | 10824 | SG | CYS | C | 451 | 7.643 | 49.011 | 14.532 | 1.00 | 190.59 | C |
| ATOM | 10825 | C | CYS | C | 451 | 9.336 | 52.521 | 15.788 | 1.00 | 185.56 | C |
| ATOM | 10826 | O | CYS | C | 451 | 10.513 | 52.134 | 15.640 | 1.00 | 185.56 | C |
| ATOM | 10827 | OXT | CYS | C | 451 | 8.992 | 53.637 | 16.230 | 1.00 | 162.96 | C |
| ATOM | 10828 | CB | ARG | D | 1 | 65.351 | 23.727 | −44.290 | 1.00 | 145.45 | D |
| ATOM | 10829 | CG | ARG | D | 1 | 64.219 | 22.825 | −44.813 | 1.00 | 145.45 | D |
| ATOM | 10830 | CD | ARG | D | 1 | 63.966 | 22.951 | −46.321 | 1.00 | 145.45 | D |
| ATOM | 10831 | NE | ARG | D | 1 | 64.717 | 21.977 | −47.118 | 1.00 | 145.45 | D |
| ATOM | 10832 | CZ | ARG | D | 1 | 64.538 | 20.658 | −47.067 | 1.00 | 145.45 | D |
| ATOM | 10833 | NH1 | ARG | D | 1 | 63.630 | 20.132 | −46.255 | 1.00 | 145.45 | D |
| ATOM | 10834 | NH2 | ARG | D | 1 | 65.275 | 19.858 | −47.827 | 1.00 | 145.45 | D |
| ATOM | 10835 | C | ARG | D | 1 | 67.140 | 21.998 | −44.719 | 1.00 | 200.84 | D |
| ATOM | 10836 | O | ARG | D | I | 66.615 | 21.129 | −45.416 | 1.00 | 200.84 | D |
| ATOM | 10837 | N | ARG | D | 1 | 66.780 | 23.889 | −46.310 | 1.00 | 200.84 | D |
| ATOM | 10838 | CA | ARG | D | 1 | 66.750 | 23.474 | −44.878 | 1.00 | 200.84 | D |
| ATOM | 10839 | N | THR | D | 2 | 68.069 | 21.722 | −43.807 | 1.00 | 200.84 | D |
| ATOM | 10840 | CA | THE | D | 2 | 68.533 | 20.353 | −43.562 | 1.00 | 200.84 | D |
| ATOM | 10841 | CB | THR | D | 2 | 69.887 | 20.339 | −42.792 | 1.00 | 116.36 | D |
| ATOM | 10842 | OG1 | THR | D | 2 | 70.865 | 21.099 | −43.511 | 1.00 | 115.10 | D |
| ATOM | 10843 | CG2 | THR | D | 2 | 70.390 | 18.908 | −42.614 | 1.00 | 118.25 | D |
| ATOM | 10844 | C | THR | D | 2 | 67.529 | 19.565 | −42.723 | 1.00 | 200.84 | D |
| ATOM | 10845 | O | THR | D | 2 | 67.354 | 19.855 | −41.538 | 1.00 | 200.84 | D |
| ATOM | 10846 | N | PRO | D | 3 | 66.845 | 18.568 | −43.319 | 1.00 | 200.84 | D |
| ATOM | 10847 | CD | PRO | D | 3 | 66.759 | 18.106 | −44.718 | 1.00 | 92.56 | D |
| ATOM | 10848 | CA | PRO | D | 3 | 65.905 | 17.836 | −42.469 | 1.00 | 200.84 | D |
| ATOM | 10849 | CB | PRO | D | 3 | 65.552 | 16.621 | −43.323 | 1.00 | 90.80 | D |
| ATOM | 10850 | CG | PRO | D | 3 | 65.507 | 17.219 | −44.696 | 1.00 | 90.67 | D |
| ATOM | 10851 | C | PRO | D | 3 | 66.618 | 17.484 | −41.161 | 1.00 | 200.84 | D |
| ATOM | 10852 | O | PRO | D | 3 | 66.003 | 17.483 | −40.092 | 1.00 | 200.84 | D |
| ATOM | 10853 | N | SER | D | 4 | 67.923 | 17.214 | −41.265 | 1.00 | 123.46 | D |
| ATOM | 10854 | CA | SER | D | 4 | 68.770 | 16.900 | −40.110 | 1.00 | 120.03 | D |
| ATOM | 10855 | CB | SER | D | 4 | 68.010 | 16.078 | −39.059 | 1.00 | 143.98 | D |
| ATOM | 10856 | OG | SER | D | 4 | 68.001 | 14.698 | −39.382 | 1.00 | 145.93 | D |
| ATOM | 10857 | C | SER | D | 4 | 70.053 | 16.154 | −40.459 | 1.00 | 120.37 | D |
| ATOM | 10858 | O | SER | D | 4 | 71.067 | 16.342 | −39.789 | 1.00 | 120.52 | D |
| ATOM | 10859 | N | ASP | D | 5 | 70.000 | 15.319 | −41.499 | 1.00 | 64.91 | D |
| ATOM | 10860 | CA | ASP | D | 5 | 71.136 | 14.492 | −41.941 | 1.00 | 65.02 | D |
| ATOM | 10861 | CB | ASP | D | 5 | 72.435 | 14.984 | −41.293 | 1.00 | 171.08 | D |
| ATOM | 10862 | CG | ASP | D | 5 | 73.630 | 14.141 | −41.656 | 1.00 | 171.08 | D |
| ATOM | 10863 | OD1 | ASP | D | 5 | 74.698 | 14.350 | −41.044 | 1.00 | 171.08 | D |
| ATOM | 10864 | OD2 | ASP | D | 5 | 73.512 | 13.278 | −42.549 | 1.00 | 169.64 | D |
| ATOM | 10865 | C | ASP | D | 5 | 70.877 | 13.014 | −41.560 | 1.00 | 62.52 | D |
| ATOM | 10866 | O | ASP | D | 5 | 71.426 | 12.092 | −42.172 | 1.00 | 66.03 | D |
| ATOM | 10867 | N | LYS | D | 6 | 70.026 | 12.809 | −40.552 | 1.00 | 58.25 | D |
| ATOM | 10868 | CA | LYS | D | 6 | 69.657 | 11.480 | −40.062 | 1.00 | 54.63 | D |
| ATOM | 10869 | CB | LYS | D | 6 | 68.923 | 11.584 | −38.728 | 1.00 | 55.50 | D |
| ATOM | 10870 | CG | LYS | D | 6 | 69.704 | 12.000 | −37.516 | 1.00 | 61.91 | D |
| ATOM | 10871 | CD | LYS | D | 6 | 68.727 | 12.153 | −36.350 | 1.00 | 65.52 | D |
| ATOM | 10872 | CE | LYS | D | 6 | 69.443 | 12.365 | −35.037 | 1.00 | 68.79 | D |
| ATOM | 10873 | NZ | LYS | D | 6 | 70.252 | 11.165 | −34.670 | 1.00 | 69.98 | D |
| ATOM | 10874 | C | LYS | D | 6 | 68.698 | 10.738 | −40.991 | 1.00 | 49.96 | D |
| ATOM | 10875 | O | LYS | D | 6 | 67.702 | 11.311 | −41.451 | 1.00 | 53.84 | D |
| ATOM | 10876 | N | PRO | D | 7 | 68.978 | 9.459 | −41.291 | 1.00 | 44.27 | D |
| ATOM | 10877 | CD | PRO | D | 7 | 70.189 | 8.650 | −41.119 | 1.00 | 34.17 | D |
| ATOM | 10878 | CA | PRO | D | 7 | 68.016 | 8.772 | −42.161 | 1.00 | 42.31 | D |
| ATOM | 10879 | CB | PRO | D | 7 | 68.622 | 7.376 | −42.348 | 1.00 | 36.17 | D |
| ATOM | 10880 | CG | PRO | D | 7 | 69.621 | 7.255 | −41.228 | 1.00 | 35.04 | D |
| ATOM | 10881 | C | PRO | D | 7 | 66.696 | 8.730 | −41.398 | 1.00 | 40.37 | D |
| ATOM | 10882 | O | PRO | D | 7 | 66.666 | 8.417 | −40.202 | 1.00 | 41.38 | D |
| ATOM | 10883 | N | VAL | D | 8 | 65.610 | 9.060 | −42.092 | 1.00 | 31.08 | D |
| ATOM | 10884 | CA | VAL | D | 8 | 64.304 | 9.107 | −41.459 | 1.00 | 32.24 | D |
| ATOM | 10885 | CB | VAL | D | 8 | 64.085 | 10.512 | −40.770 | 1.00 | 28.08 | D |
| ATOM | 10886 | CG1 | VAL | D | 8 | 64.222 | 11.625 | −41.787 | 1.00 | 28.66 | D |
| ATOM | 10887 | CG2 | VAL | D | 8 | 62.726 | 10.579 | −40.108 | 1.00 | 30.78 | D |
| ATOM | 10888 | C | VAL | D | 8 | 63.209 | 8.880 | −42.479 | 1.00 | 34.74 | D |
| ATOM | 10889 | O | VAL | D | 8 | 63.418 | 9.074 | −43.680 | 1.00 | 33.98 | D |
| ATOM | 10890 | N | ALA | D | 9 | 62.038 | 8.485 | −41.994 | 1.00 | 34.59 | D |
| ATOM | 10891 | CA | ALA | D | 9 | 60.909 | 8.279 | −42.873 | 1.00 | 40.83 | D |
| ATOM | 10892 | CB | ALA | D | 9 | 61.101 | 7.027 | −43.683 | 1.00 | 32.49 | D |
| ATOM | 10893 | C | ALA | D | 9 | 59.635 | 8.183 | −42.089 | 1.00 | 41.81 | D |
| ATOM | 10894 | O | ALA | D | 9 | 59.598 | 7.568 | −41.027 | 1.00 | 39.79 | D |
| ATOM | 10895 | N | HIS | D | 10 | 58.595 | 8.821 | −42.606 | 1.00 | 35.76 | D |
| ATOM | 10896 | CA | HIS | D | 10 | 57.274 | 8.758 | −41.998 | 1.00 | 36.63 | D |
| ATOM | 10897 | CB | HIS | D | 10 | 56.955 | 10.011 | −41.197 | 1.00 | 35.19 | D |
| ATOM | 10898 | CG | HIS | D | 10 | 55.617 | 9.959 | −40.527 | 1.00 | 36.68 | D |
| ATOM | 10899 | CD2 | HIS | D | 10 | 54.421 | 10.482 | −40.885 | 1.00 | 34.41 | D |
| ATOM | 10900 | ND1 | HIS | D | 10 | 55.391 | 9.254 | −39.366 | 1.00 | 36.82 | D |
| ATOM | 10901 | CE1 | HIS | D | 10 | 54.113 | 9.344 | −39.037 | 1.00 | 37.17 | D |
| ATOM | 10902 | NE2 | HIS | D | 10 | 53.503 | 10.084 | −39.943 | 1.00 | 34.65 | D |
| ATOM | 10903 | C | HIS | D | 10 | 56.330 | 8.655 | −43.181 | 1.00 | 34.84 | D |
| ATOM | 10904 | O | HIS | D | 10 | 55.984 | 9.651 | −43.807 | 1.00 | 33.00 | D |
| ATOM | 10905 | N | VAL | D | 11 | 55.950 | 7.437 | −43.518 | 1.00 | 36.99 | D |
| ATOM | 10906 | CA | VAL | D | 11 | 55.057 | 7.234 | −44.646 | 1.00 | 39.57 | D |
| ATOM | 10907 | CB | VAL | D | 11 | 55.516 | 6.047 | −45.513 | 1.00 | 31.92 | D |
| ATOM | 10908 | CG1 | VAL | D | 11 | 56.892 | 6.352 | −46.060 | 1.00 | 27.98 | D |
| ATOM | 10909 | CG2 | VAL | D | 11 | 55.522 | 4.759 | −44.695 | 1.00 | 33.90 | D |
| ATOM | 10910 | C | VAL | D | 11 | 53.634 | 7.018 | −44.158 | 1.00 | 38.18 | D |
| ATOM | 10911 | O | VAL | D | 11 | 53.394 | 6.554 | −43.040 | 1.00 | 35.19 | D |
| ATOM | 10912 | N | VAL | D | 12 | 52.689 | 7.360 | −45.013 | 1.00 | 37.62 | D |
| ATOM | 10913 | CA | VAL | D | 12 | 51.304 | 7.262 | −44.641 | 1.00 | 35.79 | D |
| ATOM | 10914 | CB | VAL | D | 12 | 50.733 | 8.668 | −44.550 | 1.00 | 48.43 | D |
| ATOM | 10915 | CG1 | VAL | D | 12 | 50.554 | 9.239 | −45.947 | 1.00 | 42.23 | D |
| ATOM | 10916 | CG2 | VAL | D | 12 | 49.444 | 8.651 | −43.770 | 1.00 | 51.90 | D |
| ATOM | 10917 | C | VAL | D | 12 | 50.552 | 6.417 | −45.660 | 1.00 | 33.75 | D |
| ATOM | 10918 | O | VAL | D | 12 | 51.033 | 6.217 | −46.780 | 1.00 | 33.59 | D |
| ATOM | 10919 | N | ALA | D | 13 | 49.381 | 5.916 | −45.266 | 1.00 | 44.10 | D |
| ATOM | 10920 | CA | ALA | D | 13 | 48.574 | 5.056 | −46.141 | 1.00 | 43.77 | D |
| ATOM | 10921 | CB | ALA | D | 13 | 47.522 | 4.308 | −45.319 | 1.00 | 24.47 | D |
| ATOM | 10922 | C | ALA | D | 13 | 47.896 | 5.791 | −47.277 | 1.00 | 47.75 | D |
| ATOM | 10923 | O | ALA | D | 13 | 47.396 | 6.891 | −47.102 | 1.00 | 45.26 | D |
| ATOM | 10924 | N | ASN | D | 14 | 47.883 | 5.161 | −48.441 | 1.00 | 45.32 | D |
| ATOM | 10925 | CA | ASN | D | 14 | 47.252 | 5.719 | −49.632 | 1.00 | 46.44 | D |
| ATOM | 10926 | CB | ASN | D | 14 | 47.849 | 5.066 | −50.869 | 1.00 | 42.59 | D |
| ATOM | 10927 | CG | ASN | D | 14 | 47.245 | 5.586 | −52.159 | 1.00 | 44.58 | D |
| ATOM | 10928 | OD1 | ASN | D | 14 | 47.578 | 5.089 | −53.242 | 1.00 | 44.16 | D |
| ATOM | 10929 | ND2 | ASN | D | 14 | 46.364 | 6.590 | −52.059 | 1.00 | 46.70 | D |
| ATOM | 10930 | C | ASN | D | 14 | 45.750 | 5.438 | −49.607 | 1.00 | 49.32 | D |
| ATOM | 10931 | O | ASN | D | 14 | 45.343 | 4.285 | −49.693 | 1.00 | 49.84 | D |
| ATOM | 10932 | N | PRO | D | 15 | 44.909 | 6.484 | −49.517 | 1.00 | 59.70 | D |
| ATOM | 10933 | CD | PRO | D | 15 | 45.227 | 7.918 | −49.448 | 1.00 | 38.07 | D |
| ATOM | 10934 | CA | PRO | D | 15 | 43.458 | 6.287 | −49.483 | 1.00 | 63.30 | D |
| ATOM | 10935 | CB | PRO | D | 15 | 42.927 | 7.647 | −49.024 | 1.00 | 42.41 | D |
| ATOM | 10936 | CG | PRO | D | 15 | 44.145 | 8.414 | −48.554 | 1.00 | 40.68 | D |
| ATOM | 10937 | C | PRO | D | 15 | 42.887 | 5.905 | −50.846 | 1.00 | 64.51 | D |
| ATOM | 10938 | O | PRO | D | 15 | 41.805 | 5.335 | −50.938 | 1.00 | 66.54 | D |
| ATOM | 10939 | N | GLN | D | 16 | 43.621 | 6.227 | −51.901 | 1.00 | 84.35 | D |
| ATOM | 10940 | CA | GLN | D | 16 | 43.186 | 5.936 | −53.255 | 1.00 | 83.30 | D |
| ATOM | 10941 | CB | GLN | D | 16 | 43.941 | 6.843 | −54.230 | 1.00 | 114.94 | D |
| ATOM | 10942 | CG | GLN | D | 16 | 43.289 | 7.019 | −55.591 | 1.00 | 117.87 | D |
| ATOM | 10943 | CD | GLN | D | 16 | 41.896 | 7.615 | −55.493 | 1.00 | 119.66 | D |
| ATOM | 10944 | OE1 | GLN | D | 16 | 41.667 | 8.550 | −54.728 | 1.00 | 121.78 | D |
| ATOM | 10945 | NE2 | GLN | D | 16 | 40.960 | 7.080 | −56.277 | 1.00 | 118.31 | D |
| ATOM | 10946 | C | GLN | D | 16 | 43.403 | 4.461 | −53.631 | 1.00 | 79.97 | D |
| ATOM | 10947 | O | GLN | D | 16 | 43.140 | 4.056 | −54.767 | 1.00 | 80.54 | D |
| ATOM | 10948 | N | ALA | D | 17 | 43.882 | 3.654 | −52.688 | 1.00 | 73.04 | D |
| ATOM | 10949 | CA | ALA | D | 17 | 44.119 | 2.237 | −52.960 | 1.00 | 76.80 | D |
| ATOM | 10950 | CB | ALA | D | 17 | 45.590 | 1.898 | −52.760 | 1.00 | 52.04 | D |
| ATOM | 10951 | C | ALA | D | 17 | 43.258 | 1.370 | −52.057 | 1.00 | 81.30 | D |
| ATOM | 10952 | O | ALA | D | 17 | 43.771 | 0.618 | −51.229 | 1.00 | 82.94 | D |
| ATOM | 10953 | N | GLU | D | 18 | 41.944 | 1.481 | −52.227 | 1.00 | 79.32 | D |
| ATOM | 10954 | CA | GLU | D | 18 | 40.993 | 0.719 | −51.428 | 1.00 | 81.32 | D |
| ATOM | 10955 | CB | GLU | D | 18 | 39.584 | 0.887 | −51.992 | 1.00 | 122.42 | D |
| ATOM | 10956 | CG | GLU | D | 18 | 39.437 | 0.386 | −53.421 | 1.00 | 122.42 | D |
| ATOM | 10957 | CD | GLU | D | 18 | 38.001 | 0.430 | −53.912 | 1.00 | 122.42 | D |
| ATOM | 10958 | OE1 | GLU | D | 18 | 37.146 | −0.246 | −53.302 | 1.00 | 122.42 | D |
| ATOM | 10959 | OE2 | GLU | D | 18 | 37.726 | 1.138 | −54.906 | 1.00 | 122.42 | D |
| ATOM | 10960 | C | GLU | D | 18 | 41.331 | −0.768 | −51.361 | 1.00 | 80.98 | D |
| ATOM | 10961 | O | GLU | D | 18 | 41.896 | −1.337 | −52.301 | 1.00 | 80.59 | D |
| ATOM | 10962 | N | GLY | D | 19 | 40.986 | −1.381 | −50.233 | 1.00 | 74.79 | D |
| ATOM | 10963 | CA | GLY | D | 19 | 41.227 | −2.797 | −50.050 | 1.00 | 72.28 | D |
| ATOM | 10964 | C | GLY | D | 19 | 42.643 | −3.223 | −49.708 | 1.00 | 70.78 | D |
| ATOM | 10965 | O | GLY | D | 19 | 42.871 | −4.391 | −49.398 | 1.00 | 70.79 | D |
| ATOM | 10966 | N | GLN | D | 20 | 43.599 | −2.302 | −49.759 | 1.00 | 70.44 | D |
| ATOM | 10967 | CA | GLN | D | 20 | 44.982 | −2.646 | −49.443 | 1.00 | 66.01 | D |
| ATOM | 10968 | CB | GLN | D | 20 | 45.741 | −2.940 | −50.725 | 1.00 | 89.33 | D |
| ATOM | 10969 | CG | GLN | D | 20 | 45.638 | −1.827 | −51.738 | 1.00 | 90.31 | D |
| ATOM | 10970 | CD | GLN | D | 20 | 46.340 | −2.165 | −53.029 | 1.00 | 90.07 | D |
| ATOM | 10971 | OE1 | GLN | D | 20 | 46.001 | −3.145 | −53.687 | 1.00 | 89.72 | D |
| ATOM | 10972 | NE2 | GLN | D | 20 | 47.328 | −1.356 | −53.400 | 1.00 | 89.52 | D |
| ATOM | 10973 | C | GLN | D | 20 | 45.694 | −1.553 | −48.656 | 1.00 | 60.75 | D |
| ATOM | 10974 | O | GLN | D | 20 | 45.182 | −0.447 | −48.503 | 1.00 | 59.46 | D |
| ATOM | 10975 | N | LEU | D | 21 | 46.877 | −1.871 | −48.150 | 1.00 | 51.20 | D |
| ATOM | 10976 | CA | LEU | D | 21 | 47.648 | −0.912 | −47.367 | 1.00 | 47.66 | D |
| ATOM | 10977 | CB | LEU | D | 21 | 47.969 | −1.497 | −45.985 | 1.00 | 46.13 | D |
| ATOM | 10978 | CG | LEU | D | 21 | 48.855 | −0.686 | −45.036 | 1.00 | 44.91 | D |
| ATOM | 10979 | CD1 | LEU | D | 21 | 48.365 | 0.734 | −44.960 | 1.00 | 42.21 | D |
| ATOM | 10980 | CD2 | LEU | D | 21 | 48.850 | −1.337 | −43.654 | 1.00 | 46.95 | D |
| ATOM | 10981 | C | LEU | D | 21 | 48.929 | −0.560 | −48.093 | 1.00 | 46.27 | D |
| ATOM | 10982 | O | LEU | D | 21 | 49.936 | −1.234 | −47.959 | 1.00 | 41.59 | D |
| ATOM | 10983 | N | GLN | D | 22 | 48.887 | 0.505 | −48.869 | 1.00 | 37.53 | D |
| ATOM | 10984 | CA | GLN | D | 22 | 50.044 | 0.938 | −49.642 | 1.00 | 42.13 | D |
| ATOM | 10985 | CB | GLN | D | 22 | 49.607 | 1.152 | −51.094 | 1.00 | 69.41 | D |
| ATOM | 10986 | CG | GLN | D | 22 | 50.637 | 1.773 | −52.000 | 1.00 | 76.18 | D |
| ATOM | 10987 | CD | GLN | D | 22 | 50.031 | 2.243 | −53.301 | 1.00 | 82.40 | D |
| ATOM | 10988 | OE1 | GLN | D | 22 | 50.693 | 2.888 | −54.108 | 1.00 | 87.63 | D |
| ATOM | 10989 | NE2 | GLN | D | 22 | 48.759 | 1.921 | −53.511 | 1.00 | 83.16 | D |
| ATOM | 10990 | C | GLN | D | 22 | 50.616 | 2.226 | −49.048 | 1.00 | 39.52 | D |
| ATOM | 10991 | O | GLN | D | 22 | 49.901 | 3.216 | −48.876 | 1.00 | 38.47 | D |
| ATOM | 10992 | N | TRP | D | 23 | 51.903 | 2.223 | −48.740 | 1.00 | 44.05 | D |
| ATOM | 10993 | CA | TRP | D | 23 | 52.529 | 3.397 | −48.143 | 1.00 | 37.93 | D |
| ATOM | 10994 | CB | TRP | D | 23 | 53.708 | 2.976 | −47.256 | 1.00 | 37.93 | D |
| ATOM | 10995 | CG | TRP | D | 23 | 53.372 | 1.968 | −46.212 | 1.00 | 38.45 | D |
| ATOM | 10996 | CD2 | TRP | D | 23 | 52.571 | 2.177 | −45.036 | 1.00 | 36.56 | D |
| ATOM | 10997 | CE2 | TRP | D | 23 | 52.555 | 0.959 | −44.324 | 1.00 | 38.18 | D |
| ATOM | 10998 | CE3 | TRP | D | 23 | 51.868 | 3.275 | −44.516 | 1.00 | 36.25 | D |
| ATOM | 10999 | CD1 | TRP | D | 23 | 53.787 | 0.681 | −46.169 | 1.00 | 37.92 | D |
| ATOM | 11000 | NE1 | TRP | D | 23 | 53.304 | 0.063 | −45.036 | 1.00 | 37.01 | D |
| ATOM | 11001 | CZ2 | TRP | D | 23 | 51.870 | 0.806 | −43.123 | 1.00 | 40.24 | D |
| ATOM | 11002 | CZ3 | TRP | D | 23 | 51.186 | 3.123 | −43.319 | 1.00 | 40.69 | D |
| ATOM | 11003 | CH2 | TRP | D | 23 | 51.195 | 1.896 | −42.637 | 1.00 | 38.50 | D |
| ATOM | 11004 | C | TRP | D | 23 | 53.050 | 4.416 | −49.147 | 1.00 | 38.43 | D |
| ATOM | 11005 | O | TRP | D | 23 | 53.653 | 4.044 | −50.153 | 1.00 | 39.71 | D |
| ATOM | 11006 | N | LEU | D | 24 | 52.823 | 5.700 | −48.874 | 1.00 | 37.51 | D |
| ATOM | 11007 | CA | LEU | D | 24 | 53.342 | 6.748 | −49.744 | 1.00 | 35.20 | D |
| ATOM | 11008 | CB | LEU | D | 24 | 52.250 | 7.457 | −50.540 | 1.00 | 57.62 | D |
| ATOM | 11009 | CG | LEU | D | 24 | 50.869 | 6.865 | −50.709 | 1.00 | 58.35 | D |
| ATOM | 11010 | CD1 | LEU | D | 24 | 50.141 | 7.006 | −49.401 | 1.00 | 59.97 | D |
| ATOM | 11011 | CD2 | LEU | D | 24 | 50.120 | 7.609 | −51.791 | 1.00 | 53.46 | D |
| ATOM | 11012 | C | LEU | D | 24 | 54.049 | 7.789 | −48.914 | 1.00 | 42.06 | D |
| ATOM | 11013 | O | LEU | D | 24 | 53.798 | 7.919 | −47.723 | 1.00 | 39.04 | D |
| ATOM | 11014 | N | ASN | D | 25 | 54.927 | 8.539 | −49.559 | 1.00 | 35.31 | D |
| ATOM | 11015 | CA | ASN | D | 25 | 55.670 | 9.585 | −48.885 | 1.00 | 36.75 | D |
| ATOM | 11016 | CB | ASN | D | 25 | 57.161 | 9.271 | −48.939 | 1.00 | 36.28 | D |
| ATOM | 11017 | CG | ASN | D | 25 | 57.721 | 9.444 | −50.316 | 1.00 | 37.85 | D |
| ATOM | 11018 | OD1 | ASN | D | 25 | 58.529 | 10.338 | −50.555 | 1.00 | 41.84 | D |
| ATOM | 11019 | ND2 | ASN | D | 25 | 57.275 | 8.601 | −51.249 | 1.00 | 34.25 | D |
| ATOM | 11020 | C | ASN | D | 25 | 55.417 | 10.918 | −49.580 | 1.00 | 37.28 | D |
| ATOM | 11021 | O | ASN | D | 25 | 55.911 | 11.951 | −49.145 | 1.00 | 39.64 | D |
| ATOM | 11022 | N | ARG | D | 26 | 54.648 | 10.900 | −50.660 | 1.00 | 38.45 | D |
| ATOM | 11023 | CA | ARG | D | 26 | 54.375 | 12.122 | −51.404 | 1.00 | 39.73 | D |
| ATOM | 11024 | CB | ARG | D | 26 | 54.153 | 11.792 | −52.879 | 1.00 | 39.13 | D |
| ATOM | 11025 | CG | ARG | D | 26 | 53.065 | 10.768 | −53.136 | 1.00 | 40.04 | D |
| ATOM | 11026 | CD | ARG | D | 26 | 52.735 | 10.716 | −54.618 | 1.00 | 44.10 | D |
| ATOM | 11027 | NE | ARG | D | 26 | 51.705 | 9.731 | −54.938 | 1.00 | 45.24 | D |
| ATOM | 11028 | CZ | ARG | D | 26 | 51.942 | 8.441 | −55.105 | 1.00 | 45.80 | D |
| ATOM | 11029 | NH1 | ARG | D | 26 | 53.176 | 7.976 | −54.983 | 1.00 | 46.28 | D |
| ATOM | 11030 | NH2 | ARG | D | 26 | 50.949 | 7.626 | −55.403 | 1.00 | 44.83 | D |
| ATOM | 11031 | C | ARG | D | 26 | 53.183 | 12.928 | −50.888 | 1.00 | 37.79 | D |
| ATOM | 11032 | O | ARG | D | 26 | 52.355 | 13.391 | −51.673 | 1.00 | 37.50 | D |
| ATOM | 11033 | N | ARG | D | 27 | 53.104 | 13.113 | −49.575 | 1.00 | 37.88 | D |
| ATOM | 11034 | CA | ARG | D | 27 | 51.997 | 13.842 | −49.001 | 1.00 | 38.81 | D |
| ATOM | 11035 | CB | ARG | D | 27 | 51.026 | 12.865 | −48.342 | 1.00 | 33.70 | D |
| ATOM | 11036 | CG | ARG | D | 27 | 50.303 | 11.985 | −49.335 | 1.00 | 35.12 | D |
| ATOM | 11037 | CD | ARG | D | 27 | 49.671 | 12.831 | −50.428 | 1.00 | 37.49 | D |
| ATOM | 11038 | NE | ARG | D | 27 | 49.074 | 12.036 | −51.489 | 1.00 | 40.88 | D |
| ATOM | 11039 | CZ | ARG | D | 27 | 48.044 | 11.226 | −51.297 | 1.00 | 44.49 | D |
| ATOM | 11040 | NH1 | ARG | D | 27 | 47.552 | 10.533 | −52.323 | 1.00 | 45.36 | D |
| ATOM | 11041 | NH2 | ARG | D | 27 | 47.509 | 11.117 | −50.072 | 1.00 | 42.72 | D |
| ATOM | 11042 | C | ARG | D | 27 | 52.398 | 14.920 | −48.008 | 1.00 | 39.27 | D |
| ATOM | 11043 | O | ARG | D | 27 | 53.568 | 15.057 | −47.645 | 1.00 | 39.41 | D |
| ATOM | 11044 | N | ALA | D | 28 | 51.386 | 15.676 | −47.589 | 1.00 | 78.51 | D |
| ATOM | 11045 | CA | ALA | D | 28 | 51.504 | 16.775 | −46.644 | 1.00 | 77.41 | D |
| ATOM | 11046 | CB | ALA | D | 28 | 50.195 | 16.926 | −45.880 | 1.00 | 128.01 | D |
| ATOM | 11047 | C | ALA | D | 28 | 52.658 | 16.688 | −45.656 | 1.00 | 77.07 | D |
| ATOM | 11048 | O | ALA | D | 28 | 53.732 | 17.235 | −45.891 | 1.00 | 78.09 | D |
| ATOM | 11049 | N | ASN | D | 29 | 52.447 | 16.009 | −44.539 | 1.00 | 42.67 | D |
| ATOM | 11050 | CA | ASN | D | 29 | 53.495 | 15.940 | −43.531 | 1.00 | 41.84 | D |
| ATOM | 11051 | CB | ASN | D | 29 | 52.912 | 16.343 | −42.180 | 1.00 | 42.60 | D |
| ATOM | 11052 | CG | ASN | D | 29 | 52.431 | 17.783 | −42.166 | 1.00 | 42.66 | D |
| ATOM | 11053 | OD1 | ASN | D | 29 | 53.216 | 18.713 | −42.006 | 1.00 | 48.52 | D |
| ATOM | 11054 | ND2 | ASN | D | 29 | 51.134 | 17.972 | −42.348 | 1.00 | 39.67 | D |
| ATOM | 11055 | C | ASN | D | 29 | 54.122 | 14.561 | −43.441 | 1.00 | 40.18 | D |
| ATOM | 11056 | O | ASN | D | 29 | 54.073 | 13.904 | −42.387 | 1.00 | 37.90 | D |
| ATOM | 11057 | N | ALA | D | 30 | 54.709 | 14.121 | −44.550 | 1.00 | 39.97 | D |
| ATOM | 11058 | CA | ALA | D | 30 | 55.338 | 12.809 | −44.606 | 1.00 | 34.79 | D |
| ATOM | 11059 | CB | ALA | D | 30 | 54.702 | 11.963 | −45.700 | 1.00 | 27.80 | D |
| ATOM | 11060 | C | ALA | D | 30 | 56.833 | 12.973 | −44.852 | 1.00 | 36.19 | D |
| ATOM | 11061 | O | ALA | D | 30 | 57.298 | 14.064 | −45.160 | 1.00 | 40.34 | D |
| ATOM | 11062 | N | LEU | D | 31 | 57.581 | 11.884 | −44.702 | 1.00 | 42.44 | D |
| ATOM | 11063 | CA | LEU | D | 31 | 59.019 | 11.935 | −44.879 | 1.00 | 41.91 | D |
| ATOM | 11064 | CB | LEU | D | 31 | 59.704 | 12.181 | −43.543 | 1.00 | 31.20 | D |
| ATOM | 11065 | CG | LEU | D | 31 | 59.618 | 13.534 | −42.882 | 1.00 | 34.08 | D |
| ATOM | 11066 | CD1 | LEU | D | 31 | 60.109 | 13.405 | −41.449 | 1.00 | 31.70 | D |
| ATOM | 11067 | CD2 | LEU | D | 31 | 60.444 | 14.513 | −43.659 | 1.00 | 33.55 | D |
| ATOM | 11068 | C | LEU | D | 31 | 59.609 | 10.669 | −45.447 | 1.00 | 41.17 | D |
| ATOM | 11069 | O | LEU | D | 31 | 59.131 | 9.578 | −45.175 | 1.00 | 41.98 | D |
| ATOM | 11070 | N | LEU | D | 32 | 60.684 | 10.847 | −46.208 | 1.00 | 39.52 | D |
| ATOM | 11071 | CA | LEU | D | 32 | 61.450 | 9.767 | −46.821 | 1.00 | 39.31 | D |
| ATOM | 11072 | CB | LEU | D | 32 | 60.734 | 9.220 | −48.052 | 1.00 | 34.01 | D |
| ATOM | 11073 | CG | LEU | D | 32 | 61.471 | 8.009 | −48.617 | 1.00 | 32.59 | D |
| ATOM | 11074 | CD1 | LEU | D | 32 | 61.405 | 6.858 | −47.608 | 1.00 | 31.57 | D |
| ATOM | 11075 | CD2 | LEU | D | 32 | 60.857 | 7.604 | −49.937 | 1.00 | 32.91 | D |
| ATOM | 11076 | C | LEU | D | 32 | 62.779 | 10.415 | −47.223 | 1.00 | 38.95 | D |
| ATOM | 11077 | O | LEU | D | 32 | 62.968 | 10.811 | −48.377 | 1.00 | 44.82 | D |
| ATOM | 11078 | N | ALA | D | 33 | 63.701 | 10.528 | −46.269 | 1.00 | 40.46 | D |
| ATOM | 11079 | CA | ALA | D | 33 | 64.962 | 11.194 | −46.556 | 1.00 | 40.98 | D |
| ATOM | 11080 | CB | ALA | D | 33 | 64.901 | 12.618 | −46.059 | 1.00 | 40.57 | D |
| ATOM | 11081 | C | ALA | D | 33 | 66.241 | 10.579 | −46.051 | 1.00 | 42.77 | D |
| ATOM | 11082 | O | ALA | D | 33 | 66.254 | 9.619 | −45.282 | 1.00 | 46.95 | D |
| ATOM | 11083 | N | ASN | D | 34 | 67.331 | 11.189 | −46.499 | 1.00 | 45.40 | D |
| ATOM | 11084 | CA | ASN | D | 34 | 68.670 | 10.798 | −46.118 | 1.00 | 42.59 | D |
| ATOM | 11085 | CB | ASN | D | 34 | 68.918 | 11.222 | −44.684 | 1.00 | 44.05 | D |
| ATOM | 11086 | CG | ASN | D | 34 | 68.764 | 12.699 | −44.513 | 1.00 | 47.97 | D |
| ATOM | 11087 | OD1 | ASN | D | 34 | 69.325 | 13.467 | −45.293 | 1.00 | 50.45 | D |
| ATOM | 11088 | ND2 | ASN | D | 34 | 67.995 | 13.117 | −43.506 | 1.00 | 43.38 | D |
| ATOM | 11089 | C | ASN | D | 34 | 69.015 | 9.339 | −46.298 | 1.00 | 43.34 | D |
| ATOM | 11090 | O | ASN | D | 34 | 69.656 | 8.731 | −45.441 | 1.00 | 45.88 | D |
| ATOM | 11091 | N | GLY | D | 35 | 68.587 | 8.772 | −47.417 | 1.00 | 45.94 | D |
| ATOM | 11092 | CA | GLY | D | 35 | 68.937 | 7.399 | −47.691 | 1.00 | 40.53 | D |
| ATOM | 11093 | C | GLY | D | 35 | 67.890 | 6.320 | −47.565 | 1.00 | 40.88 | D |
| ATOM | 11094 | O | GLY | D | 35 | 67.940 | 5.358 | −48.307 | 1.00 | 42.37 | D |
| ATOM | 11095 | N | VAL | D | 36 | 66.950 | 6.431 | −46.643 | 1.00 | 29.89 | D |
| ATOM | 11096 | CA | VAL | D | 36 | 65.993 | 5.351 | −46.561 | 1.00 | 31.22 | D |
| ATOM | 11097 | CB | VAL | D | 36 | 65.148 | 5.410 | −45.240 | 1.00 | 28.38 | D |
| ATOM | 11098 | CG1 | VAL | D | 36 | 65.430 | 6.664 | −44.512 | 1.00 | 26.41 | D |
| ATOM | 11099 | CG2 | VAL | D | 36 | 63.681 | 5.247 | −45.524 | 1.00 | 28.78 | D |
| ATOM | 11100 | C | VAL | D | 36 | 65.137 | 5.357 | −47.824 | 1.00 | 35.37 | D |
| ATOM | 11101 | O | VAL | D | 36 | 64.779 | 6.410 | −48.347 | 1.00 | 36.33 | D |
| ATOM | 11102 | N | GLU | D | 37 | 64.848 | 4.162 | −48.329 | 1.00 | 41.60 | D |
| ATOM | 11103 | CA | GLU | D | 37 | 64.068 | 4.015 | −49.543 | 1.00 | 44.20 | D |
| ATOM | 11104 | CB | GLU | D | 37 | 64.875 | 3.258 | −50.598 | 1.00 | 63.74 | D |
| ATOM | 11105 | CG | GLU | D | 37 | 66.346 | 3.634 | −50.603 | 1.00 | 70.77 | D |
| ATOM | 11106 | CD | GLU | D | 37 | 67.223 | 2.639 | −51.356 | 1.00 | 73.74 | D |
| ATOM | 11107 | OE1 | GLU | D | 37 | 66.861 | 1.434 | −51.413 | 1.00 | 77.17 | D |
| ATOM | 11108 | OE2 | GLU | D | 37 | 68.287 | 3.061 | −51.870 | 1.00 | 74.30 | D |
| ATOM | 11109 | C | GLU | D | 37 | 62.789 | 3.260 | −49.251 | 1.00 | 43.09 | D |
| ATOM | 11110 | O | GLU | D | 37 | 62.699 | 2.489 | −48.292 | 1.00 | 45.55 | D |
| ATOM | 11111 | N | LEU | D | 38 | 61.788 | 3.516 | −50.077 | 1.00 | 50.21 | D |
| ATOM | 11112 | CA | LEU | D | 38 | 60.505 | 2.846 | −49.957 | 1.00 | 52.25 | D |
| ATOM | 11113 | CB | LEU | D | 38 | 59.375 | 3.868 | −49.999 | 1.00 | 38.58 | D |
| ATOM | 11114 | CG | LEU | D | 38 | 57.973 | 3.316 | −49.791 | 1.00 | 36.49 | D |
| ATOM | 11115 | CD1 | LEU | D | 38 | 57.963 | 2.389 | −48.597 | 1.00 | 34.87 | D |
| ATOM | 11116 | CD2 | LEU | D | 38 | 56.985 | 4.482 | −49.610 | 1.00 | 33.36 | D |
| ATOM | 11117 | C | LEU | D | 38 | 60.490 | 1.951 | −51.184 | 1.00 | 52.52 | D |
| ATOM | 11118 | O | LEU | D | 38 | 60.279 | 2.404 | −52.300 | 1.00 | 46.13 | D |
| ATOM | 11119 | N | ARG | D | 39 | 60.753 | 0.673 | −50.969 | 1.00 | 43.83 | D |
| ATOM | 11120 | CA | ARG | D | 39 | 60.834 | −0.292 | −52.057 | 1.00 | 50.36 | D |
| ATOM | 11121 | CB | ARG | D | 39 | 62.277 | −0.783 | −52.153 | 1.00 | 55.68 | D |
| ATOM | 11122 | CG | ARG | D | 39 | 62.608 | −1.601 | −53.351 | 1.00 | 63.38 | D |
| ATOM | 11123 | CD | ARG | D | 39 | 64.107 | −1.788 | −53.433 | 1.00 | 71.99 | D |
| ATOM | 11124 | NE | ARG | D | 39 | 64.499 | −2.300 | −54.742 | 1.00 | 78.52 | D |
| ATOM | 11125 | CZ | ARG | D | 39 | 65.753 | −2.332 | −55.190 | 1.00 | 79.32 | D |
| ATOM | 11126 | NH1 | ARG | D | 39 | 66.742 | −1.881 | −54.425 | 1.00 | 77.80 | D |
| ATOM | 11127 | NH2 | ARG | D | 39 | 66.021 | −2.795 | −56.411 | 1.00 | 81.62 | D |
| ATOM | 11128 | C | ARG | D | 39 | 59.885 | −1.459 | −51.809 | 1.00 | 52.41 | D |
| ATOM | 11129 | O | ARG | D | 39 | 59.844 | −2.025 | −50.705 | 1.00 | 55.64 | D |
| ATOM | 11130 | N | ASP | D | 40 | 59.112 | −1.809 | −52.834 | 1.00 | 52.72 | D |
| ATOM | 11131 | CA | ASP | D | 40 | 58.151 | −2.911 | −52.736 | 1.00 | 50.67 | D |
| ATOM | 11132 | CB | ASP | D | 40 | 58.888 | −4.257 | −52.759 | 1.00 | 78.61 | D |
| ATOM | 11133 | CG | ASP | D | 40 | 59.795 | −4.411 | −53.978 | 1.00 | 80.75 | D |
| ATOM | 11134 | OD1 | ASP | D | 40 | 59.300 | −4.239 | −55.112 | 1.00 | 77.63 | D |
| ATOM | 11135 | OD2 | ASP | D | 40 | 61.000 | −4.708 | −53.804 | 1.00 | 80.87 | D |
| ATOM | 11136 | C | ASP | D | 40 | 57.304 | −2.797 | −51.469 | 1.00 | 46.80 | D |
| ATOM | 11137 | O | ASP | D | 40 | 56.989 | −3.793 | −50.828 | 1.00 | 46.03 | D |
| ATOM | 11138 | N | ASN | D | 41 | 56.951 | −1.565 | −51.115 | 1.00 | 36.84 | D |
| ATOM | 11139 | CA | ASN | D | 41 | 56.132 | −1.276 | −49.940 | 1.00 | 32.59 | D |
| ATOM | 11140 | CB | ASN | D | 41 | 54.836 | −2.075 | −49.978 | 1.00 | 45.67 | D |
| ATOM | 11141 | CG | ASN | D | 41 | 53.717 | −1.381 | −49.235 | 1.00 | 40.36 | D |
| ATOM | 11142 | OD1 | ASN | D | 41 | 53.415 | −0.216 | −49.504 | 1.00 | 41.70 | D |
| ATOM | 11143 | ND2 | ASN | D | 41 | 53.094 | −2.087 | −48.298 | 1.00 | 36.46 | D |
| ATOM | 11144 | C | ASN | D | 41 | 56.825 | −1.517 | −48.603 | 1.00 | 35.90 | D |
| ATOM | 11145 | O | ASN | D | 41 | 56.165 | −1.757 | −47.585 | 1.00 | 37.41 | D |
| ATOM | 11146 | N | GLN | D | 42 | 58.153 | −1.448 | −48.607 | 1.00 | 40.92 | D |
| ATOM | 11147 | CA | GLN | D | 42 | 58.907 | −1.653 | −47.389 | 1.00 | 38.84 | D |
| ATOM | 11148 | CB | GLN | D | 42 | 59.606 | −3.009 | −47.418 | 1.00 | 52.42 | D |
| ATOM | 11149 | CG | GLN | D | 42 | 58.708 | −4.152 | −47.829 | 1.00 | 54.51 | D |
| ATOM | 11150 | CD | GLN | D | 42 | 59.440 | −5.474 | −47.883 | 1.00 | 53.97 | D |
| ATOM | 11151 | OE1 | GLN | D | 42 | 60.514 | −5.581 | −48.472 | 1.00 | 53.40 | D |
| ATOM | 11152 | NE2 | GLN | D | 42 | 58.852 | −6.495 | −47.278 | 1.00 | 55.73 | D |
| ATOM | 11153 | C | GLN | D | 42 | 59.940 | −0.567 | −47.279 | 1.00 | 37.73 | D |
| ATOM | 11154 | O | GLN | D | 42 | 60.422 | −0.065 | −48.299 | 1.00 | 38.07 | D |
| ATOM | 11155 | N | LEU | D | 43 | 60.272 | −0.189 | −46.045 | 1.00 | 31.33 | D |
| ATOM | 11156 | CA | LEU | D | 43 | 61.298 | 0.827 | −45.834 | 1.00 | 29.91 | D |
| ATOM | 11157 | CB | LEU | D | 43 | 61.054 | 1.554 | −44.509 | 1.00 | 19.89 | D |
| ATOM | 11158 | CG | LEU | D | 43 | 59.753 | 2.373 | −44.474 | 1.00 | 26.05 | D |
| ATOM | 11159 | CD1 | LEU | D | 43 | 59.502 | 2.872 | −43.069 | 1.00 | 26.14 | D |
| ATOM | 11160 | CD2 | LEU | D | 43 | 59.826 | 3.516 | −45.464 | 1.00 | 22.44 | D |
| ATOM | 11161 | C | LEU | D | 43 | 62.644 | 0.099 | −45.812 | 1.00 | 31.93 | D |
| ATOM | 11162 | O | LEU | D | 43 | 62.762 | −0.981 | −45.249 | 1.00 | 28.55 | D |
| ATOM | 11163 | N | VAL | D | 44 | 63.649 | 0.679 | −46.445 | 1.00 | 33.61 | D |
| ATOM | 11164 | CA | VAL | D | 44 | 64.956 | 0.048 | −46.473 | 1.00 | 33.29 | D |
| ATOM | 11165 | CB | VAL | D | 44 | 65.376 | −0.268 | −47.915 | 1.00 | 22.48 | D |
| ATOM | 11166 | CG1 | VAL | D | 44 | 66.654 | −1.081 | −47.911 | 1.00 | 23.67 | D |
| ATOM | 11167 | CG2 | VAL | D | 44 | 64.242 | −1.016 | −48.628 | 1.00 | 17.09 | D |
| ATOM | 11168 | C | VAL | D | 44 | 66.006 | 0.936 | −45.821 | 1.00 | 32.30 | D |
| ATOM | 11169 | O | VAL | D | 44 | 66.268 | 2.050 | −46.271 | 1.00 | 33.34 | D |
| ATOM | 11170 | N | VAL | D | 45 | 66.605 | 0.422 | −44.758 | 1.00 | 40.63 | D |
| ATOM | 11171 | CA | VAL | D | 45 | 67.622 | 1.134 | −43.996 | 1.00 | 46.37 | D |
| ATOM | 11172 | CB | VAL | D | 45 | 67.900 | 0.375 | −42.677 | 1.00 | 50.88 | D |
| ATOM | 11173 | CG1 | VAL | D | 45 | 69.128 | 0.918 | −42.000 | 1.00 | 53.45 | D |
| ATOM | 11174 | CG2 | VAL | D | 45 | 66.711 | 0.511 | −41.762 | 1.00 | 54.28 | D |
| ATOM | 11175 | C | VAL | D | 45 | 68.918 | 1.308 | −44.790 | 1.00 | 48.16 | D |
| ATOM | 11176 | O | VAL | D | 45 | 69.418 | 0.369 | −45.396 | 1.00 | 47.61 | D |
| ATOM | 11177 | N | PRO | D | 46 | 69.480 | 2.522 | −44.788 | 1.00 | 48.48 | D |
| ATOM | 11178 | CD | PRO | D | 46 | 68.818 | 3.766 | −44.350 | 1.00 | 37.84 | D |
| ATOM | 11179 | CA | PRO | D | 46 | 70.720 | 2.814 | −45.515 | 1.00 | 47.33 | D |
| ATOM | 11180 | CB | PRO | D | 46 | 70.504 | 4.250 | −45.972 | 1.00 | 37.20 | D |
| ATOM | 11181 | CG | PRO | D | 46 | 69.815 | 4.848 | −44.754 | 1.00 | 37.90 | D |
| ATOM | 11182 | C | PRO | D | 46 | 72.029 | 2.665 | −44.731 | 1.00 | 50.77 | D |
| ATOM | 11183 | O | PRO | D | 46 | 73.102 | 2.820 | −45.306 | 1.00 | 53.23 | D |
| ATOM | 11184 | N | SER | D | 47 | 71.958 | 2.382 | −43.435 | 1.00 | 60.24 | D |
| ATOM | 11185 | CA | SER | D | 47 | 73.187 | 2.249 | −42.662 | 1.00 | 57.36 | D |
| ATOM | 11186 | CB | SER | D | 47 | 73.786 | 3.634 | −42.424 | 1.00 | 56.45 | D |
| ATOM | 11187 | OG | SER | D | 47 | 72.890 | 4.447 | −41.688 | 1.00 | 57.66 | D |
| ATOM | 11188 | C | SER | D | 47 | 73.029 | 1.532 | −41.319 | 1.00 | 54.05 | D |
| ATOM | 11189 | O | SER | D | 47 | 71.929 | 1.479 | −40.766 | 1.00 | 51.19 | D |
| ATOM | 11190 | N | GLU | D | 48 | 74.140 | 0.988 | −40.806 | 1.00 | 46.90 | D |
| ATOM | 11191 | CA | GLU | D | 48 | 74.169 | 0.288 | −39.520 | 1.00 | 51.39 | D |
| ATOM | 11192 | CB | GLU | D | 48 | 75.606 | −0.098 | −39.145 | 1.00 | 165.58 | D |
| ATOM | 11193 | CG | GLU | D | 48 | 76.364 | −0.939 | −40.163 | 1.00 | 170.32 | D |
| ATOM | 11194 | CD | GLU | D | 48 | 76.054 | −2.420 | −40.057 | 1.00 | 174.67 | D |
| ATOM | 11195 | OE1 | GLU | D | 48 | 74.919 | −2.821 | −40.391 | 1.00 | 176.41 | D |
| ATOM | 11196 | OE2 | GLU | D | 48 | 76.949 | −3.183 | −39.635 | 1.00 | 172.59 | D |
| ATOM | 11197 | C | GLU | D | 48 | 73.665 | 1.288 | −38.496 | 1.00 | 49.98 | D |
| ATOM | 11198 | O | GLU | D | 48 | 73.857 | 2.492 | −38.661 | 1.00 | 55.77 | D |
| ATOM | 11199 | N | GLY | D | 49 | 73.017 | 0.808 | −37.444 | 1.00 | 39.48 | D |
| ATOM | 11200 | CA | GLY | D | 49 | 72.556 | 1.729 | −36.427 | 1.00 | 37.14 | D |
| ATOM | 11201 | C | GLY | D | 49 | 71.340 | 1.357 | −35.619 | 1.00 | 39.15 | D |
| ATOM | 11202 | O | GLY | D | 49 | 70.656 | 0.368 | −35.880 | 1.00 | 41.99 | D |
| ATOM | 11203 | N | LEU | D | 50 | 71.089 | 2.182 | −34.614 | 1.00 | 47.78 | D |
| ATOM | 11204 | CA | LEU | D | 50 | 69.954 | 2.032 | −33.722 | 1.00 | 48.98 | D |
| ATOM | 11205 | CB | LEU | D | 50 | 70.331 | 2.605 | −32.357 | 1.00 | 27.34 | D |
| ATOM | 11206 | CG | LEU | D | 50 | 70.410 | 1.685 | −31.127 | 1.00 | 28.47 | D |
| ATOM | 11207 | CD1 | LEU | D | 50 | 70.788 | 0.280 | −31.525 | 1.00 | 25.28 | D |
| ATOM | 11208 | CD2 | LEU | D | 50 | 71.400 | 2.267 | −30.131 | 1.00 | 28.86 | D |
| ATOM | 11209 | C | LEU | D | 50 | 68.808 | 2.829 | −34.346 | 1.00 | 45.36 | D |
| ATOM | 11210 | O | LEU | D | 50 | 68.986 | 3.986 | −34.720 | 1.00 | 44.08 | D |
| ATOM | 11211 | N | TYR | D | 51 | 67.638 | 2.218 | −34.478 | 1.00 | 42.37 | D |
| ATOM | 11212 | CA | TYR | D | 51 | 66.512 | 2.915 | −35.089 | 1.00 | 41.02 | D |
| ATOM | 11213 | CB | TYR | D | 51 | 66.181 | 2.333 | −36.476 | 1.00 | 37.87 | D |
| ATOM | 11214 | CG | TYR | D | 51 | 67.141 | 2.654 | −37.590 | 1.00 | 37.97 | D |
| ATOM | 11215 | CD1 | TYR | D | 51 | 68.387 | 2.057 | −37.657 | 1.00 | 38.83 | D |
| ATOM | 11216 | CE1 | TYR | D | 51 | 69.266 | 2.350 | −38.693 | 1.00 | 38.39 | D |
| ATOM | 11217 | CD2 | TYR | D | 51 | 66.790 | 3.555 | −38.587 | 1.00 | 39.53 | D |
| ATOM | 11218 | CE2 | TYR | D | 51 | 67.660 | 3.859 | −39.633 | 1.00 | 37.80 | D |
| ATOM | 11219 | CZ | TYR | D | 51 | 68.892 | 3.259 | −39.681 | 1.00 | 37.50 | D |
| ATOM | 11220 | OH | TYR | D | 51 | 69.746 | 3.580 | −40.710 | 1.00 | 37.73 | D |
| ATOM | 11221 | C | TYR | D | 51 | 65.244 | 2.834 | −34.265 | 1.00 | 41.20 | D |
| ATOM | 11222 | O | TYR | D | 51 | 64.958 | 1.811 | −33.640 | 1.00 | 42.09 | D |
| ATOM | 11223 | N | LEU | D | 52 | 64.483 | 3.920 | −34.268 | 1.00 | 35.65 | D |
| ATOM | 11224 | CA | LEU | D | 52 | 63.200 | 3.931 | −33.589 | 1.00 | 37.30 | D |
| ATOM | 11225 | CB | LEU | D | 52 | 62.896 | 5.314 | −33.011 | 1.00 | 45.12 | D |
| ATOM | 11226 | CG | LEU | D | 52 | 61.457 | 5.509 | −32.517 | 1.00 | 41.01 | D |
| ATOM | 11227 | CD1 | LEU | D | 52 | 61.065 | 4.407 | −31.562 | 1.00 | 43.04 | D |
| ATOM | 11228 | CD2 | LEU | D | 52 | 61.332 | 6.861 | −31.847 | 1.00 | 35.34 | D |
| ATOM | 11229 | C | LEU | D | 52 | 62.221 | 3.587 | −34.722 | 1.00 | 38.83 | D |
| ATOM | 11230 | O | LEU | D | 52 | 62.207 | 4.260 | −35.759 | 1.00 | 39.40 | D |
| ATOM | 11231 | N | ILE | D | 53 | 61.449 | 2.514 | −34.540 | 1.00 | 34.18 | D |
| ATOM | 11232 | CA | ILE | D | 53 | 60.493 | 2.067 | −35.547 | 1.00 | 30.78 | D |
| ATOM | 11233 | CB | ILE | D | 53 | 60.738 | 0.620 | −35.950 | 1.00 | 53.15 | D |
| ATOM | 11234 | CG2 | ILE | D | 53 | 59.765 | 0.213 | −37.027 | 1.00 | 53.79 | D |
| ATOM | 11235 | CG1 | ILE | D | 53 | 62.160 | 0.463 | −36.461 | 1.00 | 52.04 | D |
| ATOM | 11236 | CD1 | ILE | D | 53 | 62.481 | −0.957 | −36.858 | 1.00 | 47.86 | D |
| ATOM | 11237 | C | ILE | D | 53 | 59.113 | 2.143 | −34.952 | 1.00 | 30.84 | D |
| ATOM | 11238 | O | ILE | D | 53 | 58.927 | 1.766 | −33.804 | 1.00 | 33.92 | D |
| ATOM | 11239 | N | TYR | D | 54 | 58.143 | 2.620 | −35.724 | 1.00 | 39.99 | D |
| ATOM | 11240 | CA | TYR | D | 54 | 56.787 | 2.730 | −35.205 | 1.00 | 37.12 | D |
| ATOM | 11241 | CB | TYR | D | 54 | 56.642 | 4.021 | −34.387 | 1.00 | 37.92 | D |
| ATOM | 11242 | CG | TYR | D | 54 | 56.816 | 5.276 | −35.213 | 1.00 | 37.92 | D |
| ATOM | 11243 | CD1 | TYR | D | 54 | 55.734 | 5.859 | −35.870 | 1.00 | 37.92 | D |
| ATOM | 11244 | CE1 | TYR | D | 54 | 55.901 | 6.968 | −36.683 | 1.00 | 37.92 | D |
| ATOM | 11245 | CD2 | TYR | D | 54 | 58.077 | 5.846 | −35.394 | 1.00 | 37.92 | D |
| ATOM | 11246 | CE2 | TYR | D | 54 | 58.252 | 6.965 | −36.216 | 1.00 | 37.92 | D |
| ATOM | 11247 | CZ | TYR | D | 54 | 57.157 | 7.516 | −36.854 | 1.00 | 37.92 | D |
| ATOM | 11248 | OH | TYR | D | 54 | 57.314 | 8.614 | −37.664 | 1.00 | 37.92 | D |
| ATOM | 11249 | C | TYR | D | 54 | 55.754 | 2.699 | −36.320 | 1.00 | 35.71 | D |
| ATOM | 11250 | O | TYR | D | 54 | 56.068 | 2.932 | −37.489 | 1.00 | 34.56 | D |
| ATOM | 11251 | N | SER | D | 55 | 54.513 | 2.419 | −35.940 | 1.00 | 40.90 | D |
| ATOM | 11252 | CA | SER | D | 55 | 53.417 | 2.349 | −36.889 | 1.00 | 40.68 | D |
| ATOM | 11253 | CB | SER | D | 55 | 53.535 | 1.077 | −37.714 | 1.00 | 44.11 | D |
| ATOM | 11254 | OG | SER | D | 55 | 52.376 | 0.899 | −38.506 | 1.00 | 50.67 | D |
| ATOM | 11255 | C | SER | D | 55 | 52.066 | 2.343 | −36.204 | 1.00 | 43.66 | D |
| ATOM | 11256 | O | SER | D | 55 | 51.926 | 1.860 | −35.078 | 1.00 | 40.80 | D |
| ATOM | 11257 | N | GLN | D | 56 | 51.069 | 2.887 | −36.887 | 1.00 | 44.40 | D |
| ATOM | 11258 | CA | GLN | D | 56 | 49.715 | 2.888 | −36.360 | 1.00 | 45.10 | D |
| ATOM | 11259 | CB | GLN | D | 56 | 49.346 | 4.205 | −35.700 | 1.00 | 44.12 | D |
| ATOM | 11260 | CG | GLN | D | 56 | 47.980 | 4.111 | −35.060 | 1.00 | 45.72 | D |
| ATOM | 11261 | CD | GLN | D | 56 | 47.390 | 5.444 | −34.688 | 1.00 | 48.84 | D |
| ATOM | 11262 | OE1 | GLN | D | 56 | 46.840 | 6.154 | −35.536 | 1.00 | 48.37 | D |
| ATOM | 11263 | NE2 | GLN | D | 56 | 47.495 | 5.798 | −33.406 | 1.00 | 51.95 | D |
| ATOM | 11264 | C | GLN | D | 56 | 48.709 | 2.649 | −37.462 | 1.00 | 47.58 | D |
| ATOM | 11265 | O | GLN | D | 56 | 48.797 | 3.247 | −38.535 | 1.00 | 45.66 | D |
| ATOM | 11266 | N | VAL | D | 57 | 47.754 | 1.770 | −37.193 | 1.00 | 64.29 | D |
| ATOM | 11267 | CA | VAL | D | 57 | 46.697 | 1.484 | −38.146 | 1.00 | 58.66 | D |
| ATOM | 11268 | CB | VAL | D | 57 | 46.744 | 0.032 | −38.647 | 1.00 | 49.10 | D |
| ATOM | 11269 | CG1 | VAL | D | 57 | 48.086 | −0.239 | −39.286 | 1.00 | 50.30 | D |
| ATOM | 11270 | CG2 | VAL | D | 57 | 46.492 | −0.932 | −37.504 | 1.00 | 46.03 | D |
| ATOM | 11271 | C | VAL | D | 57 | 45.386 | 1.712 | −37.417 | 1.00 | 61.27 | D |
| ATOM | 11272 | O | VAL | D | 57 | 45.309 | 1.565 | −36.195 | 1.00 | 62.38 | D |
| ATOM | 11273 | N | LEU | D | 58 | 44.361 | 2.096 | −38.161 | 1.00 | 58.16 | D |
| ATOM | 11274 | CA | LEU | D | 58 | 43.060 | 2.322 | −37.566 | 1.00 | 56.61 | D |
| ATOM | 11275 | CB | LEU | D | 58 | 42.653 | 3.780 | −37.730 | 1.00 | 62.65 | D |
| ATOM | 11276 | CG | LEU | D | 58 | 41.699 | 4.349 | −36.681 | 1.00 | 64.67 | D |
| ATOM | 11277 | CD1 | LEU | D | 58 | 40.986 | 5.555 | −37.276 | 1.00 | 58.69 | D |
| ATOM | 11278 | CD2 | LEU | D | 58 | 40.692 | 3.299 | −36.250 | 1.00 | 62.45 | D |
| ATOM | 11279 | C | LEU | D | 58 | 42.060 | 1.433 | −38.285 | 1.00 | 59.58 | D |
| ATOM | 11280 | O | LEU | D | 58 | 41.831 | 1.599 | −39.477 | 1.00 | 61.21 | D |
| ATOM | 11281 | N | PHE | D | 59 | 41.481 | 0.478 | −37.569 | 1.00 | 60.22 | D |
| ATOM | 11282 | CA | PHE | D | 59 | 40.490 | −0.415 | −38.165 | 1.00 | 61.12 | D |
| ATOM | 11283 | CB | PHE | D | 59 | 40.594 | −1.817 | −37.574 | 1.00 | 49.30 | D |
| ATOM | 11284 | CG | PHE | D | 59 | 41.878 | −2.522 | −37.894 | 1.00 | 45.79 | D |
| ATOM | 11285 | CD1 | PHE | D | 59 | 42.889 | −2.631 | −36.944 | 1.00 | 44.23 | D |
| ATOM | 11286 | CD2 | PHE | D | 59 | 42.066 | −3.105 | −39.137 | 1.00 | 47.75 | D |
| ATOM | 11287 | CE1 | PHE | D | 59 | 44.063 | −3.310 | −37.223 | 1.00 | 50.15 | D |
| ATOM | 11288 | CE2 | PHE | D | 59 | 43.240 | −3.787 | −39.428 | 1.00 | 50.86 | D |
| ATOM | 11289 | CZ | PHE | D | 59 | 44.240 | −3.890 | −38.469 | 1.00 | 49.46 | D |
| ATOM | 11290 | C | PHE | D | 59 | 39.086 | 0.120 | −37.906 | 1.00 | 65.23 | D |
| ATOM | 11291 | O | PHE | D | 59 | 38.819 | 0.714 | −36.860 | 1.00 | 64.92 | D |
| ATOM | 11292 | N | LYS | D | 60 | 38.195 | −0.091 | −38.863 | 1.00 | 83.95 | D |
| ATOM | 11293 | CA | LYS | D | 60 | 36.814 | 0.362 | −38.745 | 1.00 | 85.30 | D |
| ATOM | 11294 | CB | LYS | D | 60 | 36.627 | 1.720 | −39.419 | 1.00 | 68.67 | D |
| ATOM | 11295 | CG | LYS | D | 60 | 35.194 | 2.206 | −39.436 | 1.00 | 74.86 | D |
| ATOM | 11296 | CD | LYS | D | 60 | 35.084 | 3.523 | −40.177 | 1.00 | 83.07 | D |
| ATOM | 11297 | CE | LYS | D | 60 | 33.651 | 4.021 | −40.232 | 1.00 | 85.82 | D |
| ATOM | 11298 | NZ | LYS | D | 60 | 33.586 | 5.339 | −40.926 | 1.00 | 86.16 | D |
| ATOM | 11299 | C | LYS | D | 60 | 35.901 | −0.647 | −39.411 | 1.00 | 84.81 | D |
| ATOM | 11300 | O | LYS | D | 60 | 36.198 | −1.149 | −40.497 | 1.00 | 87.56 | D |
| ATOM | 11301 | N | GLY | D | 61 | 34.788 | −0.946 | −38.758 | 1.00 | 86.26 | D |
| ATOM | 11302 | CA | GLY | D | 61 | 33.862 | −1.899 | −39.322 | 1.00 | 88.82 | D |
| ATOM | 11303 | C | GLY | D | 61 | 32.438 | −1.487 | −39.054 | 1.00 | 90.52 | D |
| ATOM | 11304 | O | GLY | D | 61 | 32.140 | −0.924 | −38.002 | 1.00 | 93.69 | D |
| ATOM | 11305 | N | GLN | D | 62 | 31.559 | −1.752 | −40.018 | 1.00 | 98.59 | D |
| ATOM | 11306 | CA | GLN | D | 62 | 30.141 | −1.433 | −39.883 | 1.00 | 98.90 | D |
| ATOM | 11307 | CB | GLN | D | 62 | 29.520 | −1.171 | −41.257 | 1.00 | 118.93 | D |
| ATOM | 11308 | CG | GLN | D | 62 | 30.194 | −0.061 | −42.051 | 1.00 | 123.06 | D |
| ATOM | 11309 | CD | GLN | D | 62 | 30.058 | 1.302 | −41.395 | 1.00 | 129.00 | D |
| ATOM | 11310 | OE1 | GLN | D | 62 | 30.500 | 1.507 | −40.266 | 1.00 | 128.60 | D |
| ATOM | 11311 | NE2 | GLN | D | 62 | 29.444 | 2.242 | −42.104 | 1.00 | 129.17 | D |
| ATOM | 11312 | C | GLN | D | 62 | 29.485 | −2.648 | −39.243 | 1.00 | 96.84 | D |
| ATOM | 11313 | O | GLN | D | 62 | 29.544 | −3.750 | −39.791 | 1.00 | 98.35 | D |
| ATOM | 11314 | N | GLY | D | 63 | 28.876 | −2.451 | −38.080 | 1.00 | 93.14 | D |
| ATOM | 11315 | CA | GLY | D | 63 | 28.236 | −3.559 | −37.396 | 1.00 | 91.13 | D |
| ATOM | 11316 | C | GLY | D | 63 | 29.202 | −4.714 | −37.209 | 1.00 | 89.21 | D |
| ATOM | 11317 | O | GLY | D | 63 | 30.401 | −4.558 | −37.432 | 1.00 | 87.25 | D |
| ATOM | 11318 | N | CYS | D | 64 | 28.691 | −5.881 | −36.823 | 1.00 | 136.58 | D |
| ATOM | 11319 | CA | CYS | D | 64 | 29.550 | −7.038 | −36.598 | 1.00 | 140.82 | D |
| ATOM | 11320 | C | CYS | D | 64 | 29.400 | −8.232 | −37.537 | 1.00 | 146.91 | D |
| ATOM | 11321 | O | CYS | D | 64 | 28.297 | −8.592 | −37.946 | 1.00 | 149.82 | D |
| ATOM | 11322 | CB | CYS | D | 64 | 29.402 | −7.512 | −35.156 | 1.00 | 165.36 | D |
| ATOM | 11323 | SG | CYS | D | 64 | 30.379 | −6.503 | −34.009 | 1.00 | 166.57 | D |
| ATOM | 11324 | N | PRO | D | 65 | 30.532 | −8.859 | −37.894 | 1.00 | 109.37 | D |
| ATOM | 11325 | CD | PRO | D | 65 | 31.889 | −8.319 | −37.714 | 1.00 | 165.57 | D |
| ATOM | 11326 | CA | PRO | D | 65 | 30.561 | −10.022 | −38.783 | 1.00 | 111.71 | D |
| ATOM | 11327 | CB | PRO | D | 65 | 32.040 | −10.142 | −39.151 | 1.00 | 168.10 | D |
| ATOM | 11328 | CG | PRO | D | 65 | 32.556 | −8.746 | −38.988 | 1.00 | 167.83 | D |
| ATOM | 11329 | C | PRO | D | 65 | 30.058 | −11.272 | −38.072 | 1.00 | 114.72 | D |
| ATOM | 11330 | O | PRO | D | 65 | 29.254 | −11.196 | −37.145 | 1.00 | 114.99 | D |
| ATOM | 11331 | N | SER | D | 66 | 30.553 | −12.422 | −38.514 | 1.00 | 130.97 | D |
| ATOM | 11332 | CA | SER | D | 66 | 30.168 | −13.697 | −37.936 | 1.00 | 134.57 | D |
| ATOM | 11333 | CB | SER | D | 66 | 30.034 | −14.748 | −39.043 | 1.00 | 140.06 | D |
| ATOM | 11334 | OG | SER | D | 66 | 29.686 | −16.014 | −38.513 | 1.00 | 137.22 | D |
| ATOM | 11335 | C | SER | D | 66 | 31.208 | −14.145 | −36.918 | 1.00 | 135.12 | D |
| ATOM | 11336 | O | SER | D | 66 | 32.230 | −13.484 | −36.727 | 1.00 | 128.54 | D |
| ATOM | 11337 | N | THR | D | 67 | 30.928 | −15.272 | −36.271 | 1.00 | 200.84 | D |
| ATOM | 11338 | CA | THR | D | 67 | 31.805 | −15.865 | −35.266 | 1.00 | 200.84 | D |
| ATOM | 11339 | CB | THR | D | 67 | 32.710 | −16.954 | −35.890 | 1.00 | 175.25 | D |
| ATOM | 11340 | OG1 | THR | D | 67 | 31.901 | −17.901 | −36.598 | 1.00 | 175.25 | D |
| ATOM | 11341 | CG2 | THR | D | 67 | 33.496 | −17.685 | −34.803 | 1.00 | 175.25 | D |
| ATOM | 11342 | C | THR | D | 67 | 32.696 | −14.863 | −34.538 | 1.00 | 200.84 | D |
| ATOM | 11343 | O | THR | D | 67 | 32.262 | −14.198 | −33.596 | 1.00 | 200.84 | D |
| ATOM | 11344 | N | HIS | D | 68 | 33.943 | −14.759 | −34.985 | 1.00 | 145.05 | D |
| ATOM | 11345 | CA | HIS | D | 68 | 34.898 | −13.858 | −34.359 | 1.00 | 140.17 | D |
| ATOM | 11346 | CB | HIS | D | 68 | 35.626 | −14.600 | −33.235 | 1.00 | 200.84 | D |
| ATOM | 11347 | CG | HIS | D | 68 | 36.226 | −13.701 | −32.200 | 1.00 | 200.84 | D |
| ATOM | 11348 | CD2 | HIS | D | 68 | 37.507 | −13.540 | −31.794 | 1.00 | 200.84 | D |
| ATOM | 11349 | ND1 | HIS | D | 68 | 35.469 | −12.845 | −31.430 | 1.00 | 200.84 | D |
| ATOM | 11350 | CE1 | HIS | D | 68 | 36.258 | −12.194 | −30.594 | 1.00 | 200.84 | D |
| ATOM | 11351 | NE2 | HIS | D | 68 | 37.499 | −12.598 | −30.794 | 1.00 | 200.84 | D |
| ATOM | 11352 | C | HIS | D | 68 | 35.904 | −13.354 | −35.388 | 1.00 | 136.00 | D |
| ATOM | 11353 | O | HIS | D | 68 | 36.309 | −14.096 | −36.281 | 1.00 | 135.45 | D |
| ATOM | 11354 | N | VAL | D | 69 | 36.297 | −12.089 | −35.259 | 1.00 | 95.05 | D |
| ATOM | 11355 | CA | VAL | D | 69 | 37.265 | −11.478 | −36.167 | 1.00 | 94.78 | D |
| ATOM | 11356 | CB | VAL | D | 69 | 36.656 | −10.277 | −36.918 | 1.00 | 122.97 | D |
| ATOM | 11357 | CG1 | VAL | D | 69 | 37.672 | −9.710 | −37.891 | 1.00 | 122.73 | D |
| ATOM | 11358 | CG2 | VAL | D | 69 | 35.400 | −10.704 | −37.657 | 1.00 | 121.23 | D |
| ATOM | 11359 | C | VAL | D | 69 | 38.486 | −10.990 | −35.395 | 1.00 | 94.37 | D |
| ATOM | 11360 | O | VAL | D | 69 | 38.357 | −10.343 | −34.356 | 1.00 | 97.93 | D |
| ATOM | 11361 | N | LEU | D | 70 | 39.670 | −11.310 | −35.906 | 1.00 | 90.01 | D |
| ATOM | 11362 | CA | LEU | D | 70 | 40.912 | −10.898 | −35.268 | 1.00 | 85.23 | D |
| ATOM | 11363 | CB | LEU | D | 70 | 41.829 | −12.100 | −35.014 | 1.00 | 67.89 | D |
| ATOM | 11364 | CG | LEU | D | 70 | 41.392 | −13.119 | −33.956 | 1.00 | 69.59 | D |
| ATOM | 11365 | CD1 | LEU | D | 70 | 42.492 | −14.146 | −33.779 | 1.00 | 64.63 | D |
| ATOM | 11366 | CD2 | LEU | D | 70 | 41.103 | −12.430 | −32.628 | 1.00 | 70.19 | D |
| ATOM | 11367 | C | LEU | D | 70 | 41.651 | −9.893 | −36.129 | 1.00 | 86.27 | D |
| ATOM | 11368 | O | LEU | D | 70 | 41.783 | −10.076 | −37.337 | 1.00 | 86.38 | D |
| ATOM | 11369 | N | LEU | D | 71 | 42.127 | −8.828 | −35.495 | 1.00 | 57.40 | D |
| ATOM | 11370 | CA | LEU | D | 71 | 42.872 | −7.789 | −36.185 | 1.00 | 57.34 | D |
| ATOM | 11371 | CB | LEU | D | 71 | 42.274 | −6.418 | −35.859 | 1.00 | 59.37 | D |
| ATOM | 11372 | CG | LEU | D | 71 | 40.809 | −6.213 | −36.251 | 1.00 | 57.04 | D |
| ATOM | 11373 | CD1 | LEU | D | 71 | 40.340 | −4.842 | −35.795 | 1.00 | 53.19 | D |
| ATOM | 11374 | CD2 | LEU | D | 71 | 40.662 | −6.346 | −37.756 | 1.00 | 57.16 | D |
| ATOM | 11375 | C | LEU | D | 71 | 44.312 | −7.851 | −35.704 | 1.00 | 61.37 | D |
| ATOM | 11376 | O | LEU | D | 71 | 44.566 | −7.844 | −34.502 | 1.00 | 60.86 | D |
| ATOM | 11377 | N | THR | D | 72 | 45.254 | −7.936 | −36.635 | 1.00 | 57.13 | D |
| ATOM | 11378 | CA | THR | D | 72 | 46.663 | −7.979 | −36.254 | 1.00 | 54.56 | D |
| ATOM | 11379 | CB | THR | D | 72 | 47.317 | −9.361 | −36.525 | 1.00 | 59.02 | D |
| ATOM | 11380 | OG1 | THR | D | 72 | 47.605 | −9.504 | −37.921 | 1.00 | 61.55 | D |
| ATOM | 11381 | CG2 | THR | D | 72 | 46.394 | −10.477 | −36.088 | 1.00 | 56.74 | D |
| ATOM | 11382 | C | THR | D | 72 | 47.462 | −6.923 | −37.002 | 1.00 | 57.19 | D |
| ATOM | 11383 | O | THR | D | 72 | 47.202 | −6.617 | −38.162 | 1.00 | 58.23 | D |
| ATOM | 11384 | N | HIS | D | 73 | 48.436 | −6.359 | −36.311 | 1.00 | 50.32 | D |
| ATOM | 11385 | CA | HIS | D | 73 | 49.301 | −5.345 | −36.874 | 1.00 | 50.77 | D |
| ATOM | 11386 | CB | HIS | D | 73 | 49.013 | −3.988 | −36.240 | 1.00 | 55.17 | D |
| ATOM | 11387 | CG | HIS | D | 73 | 49.805 | −2.869 | −36.835 | 1.00 | 58.04 | D |
| ATOM | 11388 | CD2 | HIS | D | 73 | 50.462 | −2.774 | −38.015 | 1.00 | 59.80 | D |
| ATOM | 11389 | ND1 | HIS | D | 73 | 49.981 | −1.658 | −36.200 | 1.00 | 62.65 | D |
| ATOM | 11390 | CE1 | HIS | D | 73 | 50.712 | −0.865 | −36.963 | 1.00 | 61.19 | D |
| ATOM | 11391 | NE2 | HIS | D | 73 | 51.016 | −1.518 | −38.070 | 1.00 | 61.36 | D |
| ATOM | 11392 | C | HIS | D | 73 | 50.675 | −5.815 | −36.472 | 1.00 | 49.85 | D |
| ATOM | 11393 | O | HIS | D | 73 | 50.895 | −6.181 | −35.318 | 1.00 | 45.71 | D |
| ATOM | 11394 | N | THR | D | 74 | 51.607 | −5.834 | −37.408 | 1.00 | 45.42 | D |
| ATOM | 11395 | CA | THR | D | 74 | 52.942 | −6.292 | −37.056 | 1.00 | 52.01 | D |
| ATOM | 11396 | CB | THR | D | 74 | 52.983 | −7.848 | −37.043 | 1.00 | 51.50 | D |
| ATOM | 11397 | OG1 | THR | D | 74 | 54.135 | −8.312 | −37.750 | 1.00 | 54.01 | D |
| ATOM | 11398 | CG2 | THR | D | 74 | 51.729 | −8.428 | −37.681 | 1.00 | 56.33 | D |
| ATOM | 11399 | C | THR | D | 74 | 54.055 | −5.726 | −37.941 | 1.00 | 50.86 | D |
| ATOM | 11400 | O | THR | D | 74 | 53.890 | −5.597 | −39.149 | 1.00 | 46.44 | D |
| ATOM | 11401 | N | ILE | D | 75 | 55.176 | −5.365 | −37.321 | 1.00 | 45.10 | D |
| ATOM | 11402 | CA | ILE | D | 75 | 56.317 | −4.826 | −38.048 | 1.00 | 46.40 | D |
| ATOM | 11403 | CB | ILE | D | 75 | 56.911 | −3.579 | −37.354 | 1.00 | 40.17 | D |
| ATOM | 11404 | CG2 | ILE | D | 75 | 58.164 | −3.124 | −38.072 | 1.00 | 39.66 | D |
| ATOM | 11405 | CG1 | ILE | D | 75 | 55.876 | −2.454 | −37.346 | 1.00 | 37.33 | D |
| ATOM | 11406 | CD1 | ILE | D | 75 | 56.359 | −1.163 | −36.717 | 1.00 | 40.62 | D |
| ATOM | 11407 | C | ILE | D | 75 | 57.378 | −5.909 | −38.099 | 1.00 | 49.54 | D |
| ATOM | 11408 | O | ILE | D | 75 | 57.839 | −6.394 | −37.061 | 1.00 | 46.31 | D |
| ATOM | 11409 | N | SER | D | 76 | 57.762 | −6.285 | −39.314 | 1.00 | 44.54 | D |
| ATOM | 11410 | CA | SER | D | 76 | 58.758 | −7.322 | −39.516 | 1.00 | 45.62 | D |
| ATOM | 11411 | CB | SER | D | 76 | 58.255 | −8.352 | −40.524 | 1.00 | 53.52 | D |
| ATOM | 11412 | OG | SER | D | 76 | 57.030 | −8.917 | −40.119 | 1.00 | 62.81 | D |
| ATOM | 11413 | C | SER | D | 76 | 60.068 | −6.773 | −40.036 | 1.00 | 41.62 | D |
| ATOM | 11414 | O | SER | D | 76 | 60.129 | −5.710 | −40.650 | 1.00 | 37.77 | D |
| ATOM | 11415 | N | ARG | D | 77 | 61.125 | −7.522 | −39.777 | 1.00 | 44.41 | D |
| ATOM | 11416 | CA | ARG | D | 77 | 62.440 | −7.182 | −40.273 | 1.00 | 49.59 | D |
| ATOM | 11417 | CB | ARG | D | 77 | 63.442 | −6.965 | −39.128 | 1.00 | 27.87 | D |
| ATOM | 11418 | CG | ARG | D | 77 | 64.891 | −7.146 | −39.593 | 1.00 | 30.05 | D |
| ATOM | 11419 | CD | ARG | D | 77 | 65.892 | −6.414 | −38.746 | 1.00 | 30.92 | D |
| ATOM | 11420 | NE | ARG | D | 77 | 66.704 | −7.287 | −37.904 | 1.00 | 33.36 | D |
| ATOM | 11421 | CZ | ARG | D | 77 | 68.039 | −7.290 | −37.898 | 1.00 | 36.79 | D |
| ATOM | 11422 | NH1 | ARG | D | 77 | 68.708 | −8.109 | −37.088 | 1.00 | 36.88 | D |
| ATOM | 11423 | NH2 | ARG | D | 77 | 68.711 | −6.496 | −38.718 | 1.00 | 36.65 | D |
| ATOM | 11424 | C | ARG | D | 77 | 62.911 | −8.356 | −41.129 | 1.00 | 47.93 | D |
| ATOM | 11425 | O | ARG | D | 77 | 62.771 | −9.518 | −40.747 | 1.00 | 53.68 | D |
| ATOM | 11426 | N | ILE | D | 78 | 63.417 | −8.061 | −42.312 | 1.00 | 52.10 | D |
| ATOM | 11427 | CA | ILE | D | 78 | 63.969 | −9.112 | −43.135 | 1.00 | 50.79 | D |
| ATOM | 11428 | CB | ILE | D | 78 | 63.199 | −9.289 | −44.506 | 1.00 | 72.09 | D |
| ATOM | 11429 | CG2 | ILE | D | 78 | 61.876 | −8.553 | −44.478 | 1.00 | 72.59 | D |
| ATOM | 11430 | CG1 | ILE | D | 78 | 64.045 | −8.830 | −45.682 | 1.00 | 73.76 | D |
| ATOM | 11431 | CD1 | ILE | D | 78 | 65.181 | −9.770 | −46.014 | 1.00 | 74.08 | D |
| ATOM | 11432 | C | ILE | D | 78 | 65.402 | −8.591 | −43.259 | 1.00 | 52.96 | D |
| ATOM | 11433 | O | ILE | D | 78 | 65.676 | −7.633 | −43.986 | 1.00 | 51.51 | D |
| ATOM | 11434 | N | ALA | D | 79 | 66.298 | −9.193 | −42.476 | 1.00 | 49.06 | D |
| ATOM | 11435 | CA | ALA | D | 79 | 67.700 | −8.785 | −42.434 | 1.00 | 45.66 | D |
| ATOM | 11436 | CB | ALA | D | 79 | 68.361 | −9.353 | −41.191 | 1.00 | 63.01 | D |
| ATOM | 11437 | C | ALA | D | 79 | 68.507 | −9.165 | −43.658 | 1.00 | 46.86 | D |
| ATOM | 11438 | O | ALA | D | 79 | 68.220 | −10.154 | −44.316 | 1.00 | 47.06 | D |
| ATOM | 11439 | N | VAL | D | 80 | 69.525 | −8.371 | −43.961 | 1.00 | 51.01 | D |
| ATOM | 11440 | CA | VAL | D | 80 | 70.382 | −8.647 | −45.107 | 1.00 | 53.50 | D |
| ATOM | 11441 | CB | VAL | D | 80 | 71.431 | −7.534 | −45.312 | 1.00 | 58.82 | D |
| ATOM | 11442 | CG1 | VAL | D | 80 | 72.341 | −7.875 | −46.473 | 1.00 | 57.22 | D |
| ATOM | 11443 | CG2 | VAL | D | 80 | 70.746 | −6.230 | −45.573 | 1.00 | 60.52 | D |
| ATOM | 11444 | C | VAL | D | 80 | 71.128 | −9.953 | −44.867 | 1.00 | 58.05 | D |
| ATOM | 11445 | O | VAL | D | 80 | 71.422 | −10.681 | −45.805 | 1.00 | 56.64 | D |
| ATOM | 11446 | N | SER | D | 81 | 71.437 | −10.241 | −43.607 | 1.00 | 46.39 | D |
| ATOM | 11447 | CA | SER | D | 81 | 72.155 | −11.454 | −43.265 | 1.00 | 49.25 | D |
| ATOM | 11448 | CB | SER | D | 81 | 73.101 | −11.182 | −42.101 | 1.00 | 35.39 | D |
| ATOM | 11449 | OG | SER | D | 81 | 74.099 | −10.260 | −42.478 | 1.00 | 38.43 | D |
| ATOM | 11450 | C | SER | D | 81 | 71.254 | −12.635 | −42.922 | 1.00 | 53.43 | D |
| ATOM | 11451 | O | SER | D | 81 | 71.744 | −13.671 | −42.480 | 1.00 | 53.95 | D |
| ATOM | 11452 | N | TYR | D | 82 | 69.946 | −12.480 | −43.118 | 1.00 | 51.77 | D |
| ATOM | 11453 | CA | TYR | D | 82 | 68.992 | −13.550 | −42.839 | 1.00 | 53.81 | D |
| ATOM | 11454 | CB | TYR | D | 82 | 68.833 | −13.742 | −41.325 | 1.00 | 31.73 | D |
| ATOM | 11455 | CG | TYR | D | 82 | 68.058 | −14.992 | −40.937 | 1.00 | 34.93 | D |
| ATOM | 11456 | CD1 | TYR | D | 82 | 66.894 | −14.905 | −40.175 | 1.00 | 37.22 | D |
| ATOM | 11457 | CE1 | TYR | D | 82 | 66.159 | −16.035 | −39.861 | 1.00 | 39.01 | D |
| ATOM | 11458 | CD2 | TYR | D | 82 | 68.470 | −16.251 | −41.368 | 1.00 | 37.02 | D |
| ATOM | 11459 | CE2 | TYR | D | 82 | 67.743 | −17.389 | −41.061 | 1.00 | 38.49 | D |
| ATOM | 11460 | CZ | TYR | D | 82 | 66.587 | −17.281 | −40.312 | 1.00 | 39.18 | D |
| ATOM | 11461 | OH | TYR | D | 82 | 65.849 | −18.416 | −40.036 | 1.00 | 37.60 | D |
| ATOM | 11462 | C | TYR | D | 82 | 67.644 | −13.211 | −43.469 | 1.00 | 53.24 | D |
| ATOM | 11463 | O | TYR | D | 82 | 66.698 | −12.855 | −42.768 | 1.00 | 53.42 | D |
| ATOM | 11464 | N | GLN | D | 83 | 67.572 | −13.326 | −44.797 | 1.00 | 56.59 | D |
| ATOM | 11465 | CA | GLN | D | 83 | 66.356 | −13.030 | −45.572 | 1.00 | 56.48 | D |
| ATOM | 11466 | CB | GLN | D | 83 | 66.575 | −13.336 | −47.053 | 1.00 | 59.99 | D |
| ATOM | 11467 | CG | GLN | D | 83 | 66.948 | −12.157 | −47.928 | 1.00 | 64.26 | D |
| ATOM | 11468 | CD | GLN | D | 83 | 68.391 | −11.756 | −47.770 | 1.00 | 65.30 | D |
| ATOM | 11469 | OE1 | GLN | D | 83 | 68.931 | −11.006 | −48.587 | 1.00 | 64.32 | D |
| ATOM | 11470 | NE2 | GLN | D | 83 | 69.033 | −12.254 | −46.714 | 1.00 | 62.80 | D |
| ATOM | 11471 | C | GLN | D | 83 | 65.067 | −13.740 | −45.158 | 1.00 | 54.66 | D |
| ATOM | 11472 | O | GLN | D | 83 | 64.592 | −14.615 | −45.871 | 1.00 | 52.11 | D |
| ATOM | 11473 | N | THR | D | 84 | 64.487 | −13.363 | −44.029 | 1.00 | 55.82 | D |
| ATOM | 11474 | CA | THR | D | 84 | 63.245 | −13.980 | −43.605 | 1.00 | 48.19 | D |
| ATOM | 11475 | CB | THR | D | 84 | 63.459 | −15.263 | −42.810 | 1.00 | 57.11 | D |
| ATOM | 11476 | OG1 | THR | D | 84 | 64.359 | −16.115 | −43.510 | 1.00 | 57.66 | D |
| ATOM | 11477 | CG2 | THR | D | 84 | 62.128 | −15.979 | −42.610 | 1.00 | 59.86 | D |
| ATOM | 11478 | C | THR | D | 84 | 62.539 | −13.030 | −42.682 | 1.00 | 44.70 | D |
| ATOM | 11479 | O | THR | D | 84 | 63.168 | −12.432 | −41.810 | 1.00 | 46.01 | D |
| ATOM | 11480 | N | LYS | D | 85 | 61.233 | −12.897 | −42.860 | 1.00 | 51.17 | D |
| ATOM | 11481 | CA | LYS | D | 85 | 60.458 | −12.025 | −42.007 | 1.00 | 51.03 | D |
| ATOM | 11482 | CB | LYS | D | 85 | 58.993 | −12.004 | −42.440 | 1.00 | 62.70 | D |
| ATOM | 11483 | CG | LYS | D | 85 | 58.610 | −10.905 | −43.401 | 1.00 | 59.64 | D |
| ATOM | 11484 | CD | LYS | D | 85 | 57.099 | −10.883 | −43.544 | 1.00 | 58.04 | D |
| ATOM | 11485 | CE | LYS | D | 85 | 56.623 | −9.709 | −44.379 | 1.00 | 56.34 | D |
| ATOM | 11486 | NZ | LYS | D | 85 | 57.140 | −9.758 | −45.782 | 1.00 | 54.59 | D |
| ATOM | 11487 | C | LYS | D | 85 | 60.525 | −12.549 | −40.582 | 1.00 | 50.83 | D |
| ATOM | 11488 | O | LYS | D | 85 | 60.211 | −13.713 | −40.339 | 1.00 | 51.28 | D |
| ATOM | 11489 | N | VAL | D | 86 | 60.972 | −11.703 | −39.657 | 1.00 | 46.74 | D |
| ATOM | 11490 | CA | VAL | D | 86 | 61.008 | −12.046 | −38.237 | 1.00 | 49.63 | D |
| ATOM | 11491 | CB | VAL | D | 86 | 62.463 | −12.202 | −37.663 | 1.00 | 48.24 | D |
| ATOM | 11492 | CG1 | VAL | D | 86 | 63.174 | −13.342 | −38.355 | 1.00 | 54.98 | D |
| ATOM | 11493 | CG2 | VAL | D | 86 | 63.259 | −10.933 | −37.845 | 1.00 | 49.51 | D |
| ATOM | 11494 | C | VAL | D | 86 | 60.293 | −10.868 | −37.592 | 1.00 | 50.30 | D |
| ATOM | 11495 | O | VAL | D | 86 | 60.613 | −9.709 | −37.870 | 1.00 | 54.99 | D |
| ATOM | 11496 | N | ASN | D | 87 | 59.298 | −11.153 | −36.764 | 1.00 | 56.57 | D |
| ATOM | 11497 | CA | ASN | D | 87 | 58.538 | −10.083 | −36.130 | 1.00 | 53.25 | D |
| ATOM | 11498 | CB | ASN | D | 87 | 57.262 | −10.651 | −35.521 | 1.00 | 51.64 | D |
| ATOM | 11499 | CG | ASN | D | 87 | 56.314 | −11.188 | −36.565 | 1.00 | 51.94 | D |
| ATOM | 11500 | OD1 | ASN | D | 87 | 56.526 | −11.008 | −37.761 | 1.00 | 54.55 | D |
| ATOM | 11501 | ND2 | ASN | D | 87 | 55.252 | −11.841 | −36.120 | 1.00 | 48.90 | D |
| ATOM | 11502 | C | ASN | D | 87 | 59.295 | −9.289 | −35.068 | 1.00 | 52.07 | D |
| ATOM | 11503 | O | ASN | D | 87 | 59.917 | −9.869 | −34.172 | 1.00 | 50.65 | D |
| ATOM | 11504 | N | LEU | D | 88 | 59.237 | −7.961 | −35.163 | 1.00 | 43.08 | D |
| ATOM | 11505 | CA | LEU | D | 88 | 59.895 | −7.110 | −34.183 | 1.00 | 42.07 | D |
| ATOM | 11506 | CB | LEU | D | 88 | 60.552 | −5.919 | −34.855 | 1.00 | 37.81 | D |
| ATOM | 11507 | CG | LEU | D | 88 | 61.658 | −6.211 | −35.857 | 1.00 | 34.74 | D |
| ATOM | 11508 | CD1 | LEU | D | 88 | 62.222 | −4.891 | −36.335 | 1.00 | 35.51 | D |
| ATOM | 11509 | CD2 | LEU | D | 88 | 62.743 | −7.067 | −35.204 | 1.00 | 33.31 | D |
| ATOM | 11510 | C | LEU | D | 88 | 58.868 | −6.597 | −33.200 | 1.00 | 41.31 | D |
| ATOM | 11511 | O | LEU | D | 88 | 59.103 | −6.575 | −32.000 | 1.00 | 39.78 | D |
| ATOM | 11512 | N | LEU | D | 89 | 57.723 | −6.186 | −33.728 | 1.00 | 46.95 | D |
| ATOM | 11513 | CA | LEU | D | 89 | 56.639 | −5.650 | −32.921 | 1.00 | 45.87 | D |
| ATOM | 11514 | CB | LEU | D | 89 | 56.619 | −4.122 | −33.016 | 1.00 | 34.38 | D |
| ATOM | 11515 | CG | LEU | D | 89 | 57.933 | −3.408 | −32.701 | 1.00 | 38.36 | D |
| ATOM | 11516 | CD1 | LEU | D | 89 | 57.893 | −2.031 | −33.309 | 1.00 | 38.09 | D |
| ATOM | 11517 | CD2 | LEU | D | 89 | 58.164 | −3.338 | −31.199 | 1.00 | 37.48 | D |
| ATOM | 11518 | C | LEU | D | 89 | 55.345 | −6.207 | −33.484 | 1.00 | 47.54 | D |
| ATOM | 11519 | O | LEU | D | 89 | 55.189 | −6.319 | −34.703 | 1.00 | 48.26 | D |
| ATOM | 11520 | N | SER | D | 90 | 54.412 | −6.550 | −32.603 | 1.00 | 47.16 | D |
| ATOM | 11521 | CA | SER | D | 90 | 53.143 | −7.105 | −33.038 | 1.00 | 46.50 | D |
| ATOM | 11522 | CB | SER | D | 90 | 53.324 | −8.563 | −33.421 | 1.00 | 58.61 | D |
| ATOM | 11523 | OG | SER | D | 90 | 52.080 | −9.124 | −33.786 | 1.00 | 62.83 | D |
| ATOM | 11524 | C | SER | D | 90 | 52.073 | −7.018 | −31.974 | 1.00 | 45.13 | D |
| ATOM | 11525 | O | SER | D | 90 | 52.355 | −7.142 | −30.786 | 1.00 | 44.95 | D |
| ATOM | 11526 | N | ALA | D | 91 | 50.837 | −6.816 | −32.406 | 1.00 | 46.23 | D |
| ATOM | 11527 | CA | ALA | D | 91 | 49.721 | −6.737 | −31.475 | 1.00 | 44.32 | D |
| ATOM | 11528 | CB | ALA | D | 91 | 49.475 | −5.294 | −31.068 | 1.00 | 26.27 | D |
| ATOM | 11529 | C | ALA | D | 91 | 48.473 | −7.328 | −32.115 | 1.00 | 48.29 | D |
| ATOM | 11530 | O | ALA | D | 91 | 48.372 | −7.420 | −33.336 | 1.00 | 50.16 | D |
| ATOM | 11531 | N | ILE | D | 92 | 47.528 | −7.738 | −31.281 | 1.00 | 57.76 | D |
| ATOM | 11532 | CA | ILE | D | 92 | 46.287 | −8.324 | −31.760 | 1.00 | 57.74 | D |
| ATOM | 11533 | CB | ILE | D | 92 | 46.236 | −9.823 | −31.444 | 1.00 | 58.57 | D |
| ATOM | 11534 | CG2 | ILE | D | 92 | 45.000 | −10.437 | −32.061 | 1.00 | 64.64 | D |
| ATOM | 11535 | CG1 | ILE | D | 92 | 47.492 | −10.507 | −31.980 | 1.00 | 56.68 | D |
| ATOM | 11536 | CD1 | ILE | D | 92 | 47.615 | −11.961 | −31.584 | 1.00 | 62.55 | D |
| ATOM | 11537 | C | ILE | D | 92 | 45.127 | −7.640 | −31.054 | 1.00 | 57.57 | D |
| ATOM | 11538 | O | ILE | D | 92 | 45.255 | −7.232 | −29.899 | 1.00 | 58.65 | D |
| ATOM | 11539 | N | LYS | D | 93 | 43.999 | −7.511 | −31.742 | 1.00 | 69.99 | D |
| ATOM | 11540 | CA | LYS | D | 93 | 42.834 | −6.874 | −31.150 | 1.00 | 70.64 | D |
| ATOM | 11541 | CB | LYS | D | 93 | 42.829 | −5.387 | −31.498 | 1.00 | 92.51 | D |
| ATOM | 11542 | CG | LYS | D | 93 | 42.194 | −4.508 | −30.430 | 1.00 | 96.20 | D |
| ATOM | 11543 | CD | LYS | D | 93 | 42.724 | −3.075 | −30.505 | 1.00 | 95.97 | D |
| ATOM | 11544 | CE | LYS | D | 93 | 42.197 | −2.217 | −29.356 | 1.00 | 95.87 | D |
| ATOM | 11545 | NZ | LYS | D | 93 | 42.739 | −0.823 | −29.369 | 1.00 | 98.44 | D |
| ATOM | 11546 | C | LYS | D | 93 | 41.559 | −7.550 | −31.645 | 1.00 | 72.34 | D |
| ATOM | 11547 | O | LYS | D | 93 | 41.423 | −7.836 | −32.833 | 1.00 | 70.26 | D |
| ATOM | 11548 | N | SER | D | 94 | 40.634 | −7.819 | −30.723 | 1.00 | 86.32 | D |
| ATOM | 11549 | CA | SER | D | 94 | 39.366 | −8.478 | −31.047 | 1.00 | 88.77 | D |
| ATOM | 11550 | CB | SER | D | 94 | 39.108 | −9.631 | −30.070 | 1.00 | 95.93 | D |
| ATOM | 11551 | OG | SER | D | 94 | 39.083 | −9.177 | −28.726 | 1.00 | 98.36 | D |
| ATOM | 11552 | C | SER | D | 94 | 38.216 | −7.483 | −30.978 | 1.00 | 91.36 | D |
| ATOM | 11553 | O | SER | D | 94 | 37.671 | −7.228 | −29.910 | 1.00 | 92.63 | D |
| ATOM | 11554 | N | PRO | D | 95 | 37.816 | −6.926 | −32.129 | 1.00 | 88.18 | D |
| ATOM | 11555 | CD | PRO | D | 95 | 38.235 | −7.343 | −33.480 | 1.00 | 119.26 | D |
| ATOM | 11556 | CA | PRO | D | 95 | 36.727 | −5.948 | −32.203 | 1.00 | 92.12 | D |
| ATOM | 11557 | CB | PRO | D | 95 | 36.747 | −5.530 | −33.665 | 1.00 | 122.20 | D |
| ATOM | 11558 | CG | PRO | D | 95 | 37.114 | −6.812 | −34.349 | 1.00 | 119.65 | D |
| ATOM | 11559 | C | PRO | D | 95 | 35.381 | −6.522 | −31.792 | 1.00 | 96.55 | D |
| ATOM | 11560 | O | PRO | D | 95 | 34.887 | −6.265 | −30.693 | 1.00 | 99.98 | D |
| ATOM | 11561 | N | CYS | D | 96 | 34.789 | −7.296 | −32.691 | 1.00 | 166.77 | D |
| ATOM | 11562 | CA | CYS | D | 96 | 33.505 | −7.911 | −32.422 | 1.00 | 166.56 | D |
| ATOM | 11563 | C | CYS | D | 96 | 33.689 | −9.150 | −31.568 | 1.00 | 167.66 | D |
| ATOM | 11564 | O | CYS | D | 96 | 34.189 | −10.175 | −32.033 | 1.00 | 167.96 | D |
| ATOM | 11565 | CB | CYS | D | 96 | 32.813 | −8.273 | −33.731 | 1.00 | 200.84 | D |
| ATOM | 11566 | SG | CYS | D | 96 | 32.273 | −6.814 | −34.670 | 1.00 | 200.84 | D |
| ATOM | 11567 | N | GLN | D | 97 | 33.289 | −9.036 | −30.307 | 1.00 | 200.84 | D |
| ATOM | 11568 | CA | GLN | D | 97 | 33.388 | −10.135 | −29.359 | 1.00 | 200.84 | D |
| ATOM | 11569 | CB | GLN | D | 97 | 33.292 | −9.590 | −27.931 | 1.00 | 172.22 | D |
| ATOM | 11570 | CG | GLN | D | 97 | 34.266 | −8.457 | −27.637 | 1.00 | 172.22 | D |
| ATOM | 11571 | CD | GLN | D | 97 | 33.882 | −7.661 | −26.403 | 1.00 | 172.22 | D |
| ATOM | 11572 | OE1 | GLN | D | 97 | 33.779 | −8.206 | −25.304 | 1.00 | 172.22 | D |
| ATOM | 11573 | NE2 | GLN | D | 97 | 33.666 | −6.361 | −26.582 | 1.00 | 172.22 | D |
| ATOM | 11574 | C | GLN | D | 97 | 32.245 | −11.114 | −29.627 | 1.00 | 200.84 | D |
| ATOM | 11575 | O | GLN | D | 97 | 31.590 | −11.584 | −28.697 | 1.00 | 200.84 | D |
| ATOM | 11576 | N | ARG | D | 98 | 32.012 | −11.402 | −30.908 | 1.00 | 200.84 | D |
| ATOM | 11577 | CA | ARG | D | 98 | 30.953 | −12.314 | −31.343 | 1.00 | 200.84 | D |
| ATOM | 11578 | CB | ARG | D | 98 | 31.034 | −13.627 | −30.546 | 1.00 | 190.78 | D |
| ATOM | 11579 | CG | ARG | D | 98 | 29.885 | −14.600 | −30.775 | 1.00 | 190.78 | D |
| ATOM | 11580 | CD | ARG | D | 98 | 29.827 | −15.104 | −32.205 | 1.00 | 190.78 | D |
| ATOM | 11581 | NE | ARG | D | 98 | 28.601 | −15.857 | −32.460 | 1.00 | 190.78 | D |
| ATOM | 11582 | CZ | ARG | D | 98 | 28.213 | −16.923 | −31.765 | 1.00 | 190.78 | D |
| ATOM | 11583 | NH1 | ARG | D | 98 | 27.080 | −17.539 | −32.074 | 1.00 | 190.78 | D |
| ATOM | 11584 | NH2 | ARG | D | 98 | 28.956 | −17.376 | −30.763 | 1.00 | 190.78 | D |
| ATOM | 11585 | C | ARG | D | 98 | 29.555 | −11.690 | −31.218 | 1.00 | 200.84 | D |
| ATOM | 11586 | O | ARG | D | 98 | 28.548 | −12.337 | −31.518 | 1.00 | 200.84 | D |
| ATOM | 11587 | N | GLU | D | 99 | 29.500 | −10.428 | −30.793 | 1.00 | 193.32 | D |
| ATOM | 11588 | CA | GLU | D | 99 | 28.231 | −9.716 | −30.631 | 1.00 | 193.32 | D |
| ATOM | 11589 | CB | GLU | D | 99 | 28.466 | −8.202 | −30.528 | 1.00 | 168.61 | D |
| ATOM | 11590 | CG | GLU | D | 99 | 29.898 | −7.754 | −30.793 | 1.00 | 168.61 | D |
| ATOM | 11591 | CD | GLU | D | 99 | 30.769 | −7.823 | −29.555 | 1.00 | 168.61 | D |
| ATOM | 11592 | OE1 | GLU | D | 99 | 30.684 | −8.833 | −28.828 | 1.00 | 168.61 | D |
| ATOM | 11593 | OE2 | GLU | D | 99 | 31.543 | −6.870 | −29.314 | 1.00 | 168.61 | D |
| ATOM | 11594 | C | GLU | D | 99 | 27.259 | −9.994 | −31.777 | 1.00 | 193.32 | D |
| ATOM | 11595 | O | GLU | D | 99 | 26.103 | −10.343 | −31.543 | 1.00 | 193.32 | D |
| ATOM | 11596 | N | THR | D | 100 | 27.741 | −9.837 | −33.008 | 1.00 | 200.84 | D |
| ATOM | 11597 | CA | THR | D | 100 | 26.938 | −10.060 | −34.210 | 1.00 | 200.84 | D |
| ATOM | 11598 | CB | THR | D | 100 | 26.728 | −11.573 | −34.479 | 1.00 | 183.51 | D |
| ATOM | 11599 | OG1 | THR | D | 100 | 26.135 | −12.193 | −33.332 | 1.00 | 183.79 | D |
| ATOM | 11600 | CG2 | THR | D | 100 | 28.057 | −12.252 | −34.786 | 1.00 | 184.68 | D |
| ATOM | 11601 | C | THR | D | 100 | 25.573 | −9.372 | −34.141 | 1.00 | 200.84 | D |
| ATOM | 11602 | O | THR | D | 100 | 24.730 | −9.716 | −33.311 | 1.00 | 200.84 | D |
| ATOM | 11603 | N | PRO | D | 101 | 25.337 | −8.388 | −35.026 | 1.00 | 200.84 | D |
| ATOM | 11604 | CD | PRO | D | 101 | 26.230 | −7.965 | −36.120 | 1.00 | 136.24 | D |
| ATOM | 11605 | CA | PRO | D | 101 | 24.077 | −7.641 | −35.074 | 1.00 | 200.84 | D |
| ATOM | 11606 | CB | PRO | D | 101 | 24.140 | −6.963 | −36.439 | 1.00 | 137.19 | D |
| ATOM | 11607 | CG | PRO | D | 101 | 25.595 | −6.667 | −36.577 | 1.00 | 135.22 | D |
| ATOM | 11608 | C | PRO | D | 101 | 22.840 | −8.521 | −34.912 | 1.00 | 200.84 | D |
| ATOM | 11609 | O | PRO | D | 101 | 22.516 | −9.327 | −35.786 | 1.00 | 200.84 | D |
| ATOM | 11610 | N | GLU | D | 102 | 22.159 | −8.355 | −33.782 | 1.00 | 182.51 | D |
| ATOM | 11611 | CA | GLU | D | 102 | 20.954 | −9.113 | −33.471 | 1.00 | 182.51 | D |
| ATOM | 11612 | CB | GLU | D | 102 | 20.262 | −8.486 | −32.255 | 1.00 | 191.27 | D |
| ATOM | 11613 | CG | GLU | D | 102 | 18.936 | −9.122 | −31.856 | 1.00 | 192.47 | D |
| ATOM | 11614 | CD | GLU | D | 102 | 17.802 | −8.768 | −32.802 | 1.00 | 193.78 | D |
| ATOM | 11615 | OE1 | GLU | D | 102 | 17.505 | −7.564 | −32.957 | 1.00 | 194.59 | D |
| ATOM | 11616 | OE2 | GLU | D | 102 | 17.206 | −9.696 | −33.390 | 1.00 | 192.90 | D |
| ATOM | 11617 | C | GLU | D | 102 | 19.992 | −9.173 | −34.660 | 1.00 | 179.07 | D |
| ATOM | 11618 | O | GLU | D | 102 | 19.375 | −10.208 | −34.917 | 1.00 | 176.13 | D |
| ATOM | 11619 | N | GLY | D | 103 | 19.872 | −8.063 | −35.382 | 1.00 | 140.32 | D |
| ATOM | 11620 | CA | GLY | D | 103 | 18.981 | −8.016 | −36.530 | 1.00 | 137.64 | D |
| ATOM | 11621 | C | GLY | D | 103 | 18.421 | −6.622 | −36.741 | 1.00 | 135.51 | D |
| ATOM | 11622 | O | GLY | D | 103 | 17.817 | −6.327 | −37.774 | 1.00 | 130.00 | D |
| ATOM | 11623 | N | ALA | D | 104 | 18.625 | −5.761 | −35.750 | 1.00 | 191.84 | D |
| ATOM | 11624 | CA | ALA | D | 104 | 18.151 | −4.384 | −35.810 | 1.00 | 191.84 | D |
| ATOM | 11625 | CB | ALA | D | 104 | 16.871 | −4.234 | −34.983 | 1.00 | 102.77 | D |
| ATOM | 11626 | C | ALA | D | 104 | 19.232 | −3.438 | −35.288 | 1.00 | 191.84 | D |
| ATOM | 11627 | O | ALA | D | 104 | 19.598 | −3.501 | −34.115 | 1.00 | 191.84 | D |
| ATOM | 11628 | N | GLU | D | 105 | 19.735 | −2.578 | −36.176 | 1.00 | 161.16 | D |
| ATOM | 11629 | CA | GLU | D | 105 | 20.776 | −1.586 | −35.876 | 1.00 | 161.16 | D |
| ATOM | 11630 | CB | GLU | D | 105 | 20.792 | −1.230 | −34.384 | 1.00 | 197.58 | D |
| ATOM | 11631 | CG | GLU | D | 105 | 19.560 | −0.481 | −33.906 | 1.00 | 197.58 | D |
| ATOM | 11632 | CD | GLU | D | 105 | 19.374 | 0.844 | −34.620 | 1.00 | 197.58 | D |
| ATOM | 11633 | OE1 | GLU | D | 105 | 19.152 | 0.834 | −35.849 | 1.00 | 197.58 | D |
| ATOM | 11634 | OE2 | GLU | D | 105 | 19.451 | 1.898 | −33.953 | 1.00 | 196.35 | D |
| ATOM | 11635 | C | GLU | D | 105 | 22.184 | −2.001 | −36.310 | 1.00 | 161.16 | D |
| ATOM | 11636 | O | GLU | D | 105 | 22.428 | −2.250 | −37.492 | 1.00 | 161.16 | D |
| ATOM | 11637 | N | ALA | D | 106 | 23.100 | −2.054 | −35.342 | 1.00 | 173.84 | D |
| ATOM | 11638 | CA | ALA | D | 106 | 24.502 | −2.422 | −35.560 | 1.00 | 173.84 | D |
| ATOM | 11639 | CB | ALA | D | 106 | 24.615 | −3.492 | −36.647 | 1.00 | 86.40 | D |
| ATOM | 11640 | C | ALA | D | 106 | 25.362 | −1.211 | −35.922 | 1.00 | 173.84 | D |
| ATOM | 11641 | O | ALA | D | 106 | 25.645 | −0.965 | −37.095 | 1.00 | 173.84 | D |
| ATOM | 11642 | N | LYS | D | 107 | 25.776 | −0.462 | −34.904 | 1.00 | 175.20 | D |
| ATOM | 11643 | CA | LYS | D | 107 | 26.604 | 0.729 | −35.093 | 1.00 | 169.32 | D |
| ATOM | 11644 | CB | LYS | D | 107 | 26.722 | 1.511 | −33.775 | 1.00 | 175.29 | D |
| ATOM | 11645 | CG | LYS | D | 107 | 25.495 | 2.324 | −33.373 | 1.00 | 175.24 | D |
| ATOM | 11646 | CD | LYS | D | 107 | 24.291 | 1.450 | −33.073 | 1.00 | 176.67 | D |
| ATOM | 11647 | CE | LYS | D | 107 | 23.157 | 2.278 | −32.492 | 1.00 | 172.71 | D |
| ATOM | 11648 | NZ | LYS | D | 107 | 22.796 | 3.433 | −33.358 | 1.00 | 172.97 | D |
| ATOM | 11649 | C | LYS | D | 107 | 28.006 | 0.367 | −35.584 | 1.00 | 164.80 | D |
| ATOM | 11650 | O | LYS | D | 107 | 28.391 | −0.803 | −35.583 | 1.00 | 163.93 | D |
| ATOM | 11651 | N | PRO | D | 108 | 28.784 | 1.372 | −36.022 | 1.00 | 99.29 | D |
| ATOM | 11652 | CD | PRO | D | 108 | 28.389 | 2.772 | −36.272 | 1.00 | 98.21 | D |
| ATOM | 11653 | CA | PRO | D | 108 | 30.143 | 1.128 | −36.505 | 1.00 | 98.03 | D |
| ATOM | 11654 | CB | PRO | D | 108 | 30.399 | 2.331 | −37.399 | 1.00 | 97.36 | D |
| ATOM | 11655 | CG | PRO | D | 108 | 29.702 | 3.424 | −36.658 | 1.00 | 94.74 | D |
| ATOM | 11656 | C | PRO | D | 108 | 31.125 | 1.062 | −35.333 | 1.00 | 97.26 | D |
| ATOM | 11657 | O | PRO | D | 108 | 30.834 | 1.559 | −34.244 | 1.00 | 95.44 | D |
| ATOM | 11658 | N | TRP | D | 109 | 32.281 | 0.442 | −35.557 | 1.00 | 81.24 | D |
| ATOM | 11659 | CA | TRP | D | 109 | 33.300 | 0.335 | −34.517 | 1.00 | 78.69 | D |
| ATOM | 11660 | CB | TRP | D | 109 | 33.378 | −1.090 | −33.965 | 1.00 | 96.45 | D |
| ATOM | 11661 | CG | TRP | D | 109 | 33.603 | −2.142 | −35.004 | 1.00 | 96.14 | D |
| ATOM | 11662 | CD2 | TRP | D | 109 | 34.859 | −2.543 | −35.568 | 1.00 | 95.15 | D |
| ATOM | 11663 | CE2 | TRP | D | 109 | 34.589 | −3.576 | −36.493 | 1.00 | 94.40 | D |
| ATOM | 11664 | CE3 | TRP | D | 109 | 36.185 | −2.131 | −35.382 | 1.00 | 95.25 | D |
| ATOM | 11665 | CD1 | TRP | D | 109 | 32.653 | −2.922 | −35.596 | 1.00 | 93.24 | D |
| ATOM | 11666 | NE1 | TRP | D | 109 | 33.236 | −3.787 | −36.490 | 1.00 | 91.56 | D |
| ATOM | 11667 | CZ2 | TRP | D | 109 | 35.596 | −4.203 | −37.232 | 1.00 | 90.85 | D |
| ATOM | 11668 | CZ3 | TRP | D | 109 | 37.187 | −2.756 | −36.118 | 1.00 | 95.01 | D |
| ATOM | 11669 | CH2 | TRP | D | 109 | 36.885 | −3.781 | −37.031 | 1.00 | 90.65 | D |
| ATOM | 11670 | C | TRP | D | 109 | 34.674 | 0.747 | −35.019 | 1.00 | 80.30 | D |
| ATOM | 11671 | O | TRP | D | 109 | 35.026 | 0.525 | −36.176 | 1.00 | 81.91 | D |
| ATOM | 11672 | N | TYR | D | 110 | 35.443 | 1.356 | −34.127 | 1.00 | 77.28 | D |
| ATOM | 11673 | CA | TYR | D | 110 | 36.791 | 1.801 | −34.439 | 1.00 | 75.92 | D |
| ATOM | 11674 | CB | TYR | D | 110 | 36.888 | 3.318 | −34.334 | 1.00 | 68.37 | D |
| ATOM | 11675 | CG | TYR | D | 110 | 36.027 | 4.071 | −35.314 | 1.00 | 71.92 | D |
| ATOM | 11676 | CD1 | TYR | D | 110 | 34.646 | 4.131 | −35.161 | 1.00 | 76.01 | D |
| ATOM | 11677 | CE1 | TYR | D | 110 | 33.858 | 4.864 | −36.048 | 1.00 | 76.33 | D |
| ATOM | 11678 | CD2 | TYR | D | 110 | 36.600 | 4.755 | −36.381 | 1.00 | 72.15 | D |
| ATOM | 11679 | CE2 | TYR | D | 110 | 35.828 | 5.487 | −37.271 | 1.00 | 77.52 | D |
| ATOM | 11680 | CZ | TYR | D | 110 | 34.461 | 5.541 | −37.101 | 1.00 | 77.81 | D |
| ATOM | 11681 | OH | TYR | D | 110 | 33.705 | 6.286 | −37.979 | 1.00 | 79.01 | D |
| ATOM | 11682 | C | TYR | D | 110 | 37.772 | 1.158 | −33.461 | 1.00 | 74.43 | D |
| ATOM | 11683 | O | TYR | D | 110 | 37.514 | 1.087 | −32.257 | 1.00 | 75.62 | D |
| ATOM | 11684 | N | GLU | D | 111 | 38.899 | 0.696 | −33.988 | 1.00 | 66.63 | D |
| ATOM | 11685 | CA | GLU | D | 111 | 39.917 | 0.056 | −33.165 | 1.00 | 64.65 | D |
| ATOM | 11686 | CB | GLU | D | 111 | 39.715 | −1.458 | −33.193 | 1.00 | 86.18 | D |
| ATOM | 11687 | CG | GLU | D | 111 | 39.913 | −2.114 | −31.850 | 1.00 | 93.10 | D |
| ATOM | 11688 | CD | GLU | D | 111 | 38.935 | −1.601 | −30.811 | 1.00 | 98.07 | D |
| ATOM | 11689 | OE1 | GLU | D | 111 | 37.715 | −1.734 | −31.037 | 1.00 | 98.28 | D |
| ATOM | 11690 | OE2 | GLU | D | 111 | 39.380 | −1.066 | −29.772 | 1.00 | 105.23 | D |
| ATOM | 11691 | C | GLU | D | 111 | 41.320 | 0.402 | −33.673 | 1.00 | 65.23 | D |
| ATOM | 11692 | O | GLU | D | 111 | 41.724 | −0.049 | −34.739 | 1.00 | 70.59 | D |
| ATOM | 11693 | N | PRO | D | 112 | 42.073 | 1.227 | −32.922 | 1.00 | 58.80 | D |
| ATOM | 11694 | CD | PRO | D | 112 | 41.594 | 2.103 | −31.836 | 1.00 | 55.96 | D |
| ATOM | 11695 | CA | PRO | D | 112 | 43.426 | 1.616 | −33.322 | 1.00 | 58.03 | D |
| ATOM | 11696 | CB | PRO | D | 112 | 43.511 | 3.046 | −32.829 | 1.00 | 52.03 | D |
| ATOM | 11697 | CG | PRO | D | 112 | 42.822 | 2.942 | −31.516 | 1.00 | 53.16 | D |
| ATOM | 11698 | C | PRO | D | 112 | 44.474 | 0.735 | −32.667 | 1.00 | 55.06 | D |
| ATOM | 11699 | O | PRO | D | 112 | 44.219 | 0.136 | −31.629 | 1.00 | 55.19 | D |
| ATOM | 11700 | N | ILE | D | 113 | 45.648 | 0.648 | −33.280 | 1.00 | 54.92 | D |
| ATOM | 11701 | CA | ILE | D | 113 | 46.748 | −0.142 | −32.731 | 1.00 | 56.89 | D |
| ATOM | 11702 | CB | ILE | D | 113 | 46.866 | −1.523 | −33.379 | 1.00 | 44.72 | D |
| ATOM | 11703 | CG2 | ILE | D | 113 | 48.050 | −2.250 | −32.775 | 1.00 | 40.74 | D |
| ATOM | 11704 | CG1 | ILE | D | 113 | 45.598 | −2.342 | −33.159 | 1.00 | 41.78 | D |
| ATOM | 11705 | CD1 | ILE | D | 113 | 45.709 | −3.780 | −33.704 | 1.00 | 42.26 | D |
| ATOM | 11706 | C | ILE | D | 113 | 48.067 | 0.582 | −32.985 | 1.00 | 61.11 | D |
| ATOM | 11707 | O | ILE | D | 113 | 48.360 | 0.966 | −34.115 | 1.00 | 64.51 | D |
| ATOM | 11708 | N | TYR | D | 114 | 48.858 | 0.770 | −31.935 | 1.00 | 53.61 | D |
| ATOM | 11709 | CA | TYR | D | 114 | 50.143 | 1.449 | −32.064 | 1.00 | 51.89 | D |
| ATOM | 11710 | CB | TYR | D | 114 | 50.230 | 2.636 | −31.113 | 1.00 | 41.06 | D |
| ATOM | 11711 | CG | TYR | D | 114 | 51.448 | 3.498 | −31.341 | 1.00 | 38.92 | D |
| ATOM | 11712 | CD1 | TYR | D | 114 | 51.433 | 4.513 | −32.298 | 1.00 | 43.21 | D |
| ATOM | 11713 | CE1 | TYR | D | 114 | 52.559 | 5.306 | −32.531 | 1.00 | 42.78 | D |
| ATOM | 11714 | CD2 | TYR | D | 114 | 52.632 | 3.291 | −30.611 | 1.00 | 39.09 | D |
| ATOM | 11715 | CE2 | TYR | D | 114 | 53.766 | 4.074 | −30.832 | 1.00 | 43.42 | D |
| ATOM | 11716 | CZ | TYR | D | 114 | 53.723 | 5.080 | −31.796 | 1.00 | 43.50 | D |
| ATOM | 11717 | OH | TYR | D | 114 | 54.847 | 5.849 | −32.039 | 1.00 | 43.00 | D |
| ATOM | 11718 | C | TYR | D | 114 | 51.263 | 0.470 | −31.751 | 1.00 | 51.73 | D |
| ATOM | 11719 | O | TYR | D | 114 | 51.135 | −0.388 | −30.881 | 1.00 | 54.64 | D |
| ATOM | 11720 | N | LEU | D | 115 | 52.365 | 0.612 | −32.466 | 1.00 | 49.20 | D |
| ATOM | 11721 | CA | LEU | D | 115 | 53.513 | −0.263 | −32.298 | 1.00 | 48.24 | D |
| ATOM | 11722 | CB | LEU | D | 115 | 53.537 | −1.281 | −33.436 | 1.00 | 49.22 | D |
| ATOM | 11723 | CG | LEU | D | 115 | 53.227 | −2.757 | −33.194 | 1.00 | 52.47 | D |
| ATOM | 11724 | CD1 | LEU | D | 115 | 52.120 | −2.922 | −32.166 | 1.00 | 50.85 | D |
| ATOM | 11725 | CD2 | LEU | D | 115 | 52.850 | −3.395 | −34.537 | 1.00 | 55.77 | D |
| ATOM | 11726 | C | LEU | D | 115 | 54.775 | 0.584 | −32.349 | 1.00 | 50.68 | D |
| ATOM | 11727 | O | LEU | D | 115 | 54.873 | 1.508 | −33.156 | 1.00 | 53.21 | D |
| ATOM | 11728 | N | GLY | D | 116 | 55.740 | 0.280 | −31.492 | 1.00 | 40.97 | D |
| ATOM | 11729 | CA | GLY | D | 116 | 56.965 | 1.048 | −31.516 | 1.00 | 35.31 | D |
| ATOM | 11730 | C | GLY | D | 116 | 58.060 | 0.548 | −30.599 | 1.00 | 39.16 | D |
| ATOM | 11731 | O | GLY | D | 116 | 57.784 | 0.127 | −29.484 | 1.00 | 38.04 | D |
| ATOM | 11732 | N | GLY | D | 117 | 59.304 | 0.606 | −31.067 | 1.00 | 32.94 | D |
| ATOM | 11733 | CA | GLY | D | 117 | 60.429 | 0.159 | −30.269 | 1.00 | 35.94 | D |
| ATOM | 11734 | C | GLY | D | 117 | 61.748 | 0.507 | −30.933 | 1.00 | 37.25 | D |
| ATOM | 11735 | O | GLY | D | 117 | 61.764 | 0.935 | −32.094 | 1.00 | 38.63 | D |
| ATOM | 11736 | N | VAL | D | 118 | 62.853 | 0.335 | −30.206 | 1.00 | 38.09 | D |
| ATOM | 11737 | CA | VAL | D | 118 | 64.179 | 0.631 | −30.747 | 1.00 | 40.87 | D |
| ATOM | 11738 | CB | VAL | D | 118 | 65.004 | 1.414 | −29.725 | 1.00 | 41.23 | D |
| ATOM | 11739 | CG1 | VAL | D | 118 | 66.409 | 1.660 | −30.251 | 1.00 | 42.63 | D |
| ATOM | 11740 | CG2 | VAL | D | 118 | 64.321 | 2.736 | −29.448 | 1.00 | 41.29 | D |
| ATOM | 11741 | C | VAL | D | 118 | 64.902 | −0.658 | −31.150 | 1.00 | 38.78 | D |
| ATOM | 11742 | O | VAL | D | 118 | 64.867 | −1.630 | −30.425 | 1.00 | 42.73 | D |
| ATOM | 11743 | N | PHE | D | 119 | 65.550 | −0.659 | −32.313 | 1.00 | 35.34 | D |
| ATOM | 11744 | CA | PHE | D | 119 | 66.227 | −1.852 | −32.815 | 1.00 | 36.65 | D |
| ATOM | 11745 | CB | PHE | D | 119 | 65.311 | −2.603 | −33.786 | 1.00 | 39.00 | D |
| ATOM | 11746 | CG | PHE | D | 119 | 64.041 | −3.082 | −33.169 | 1.00 | 36.60 | D |
| ATOM | 11747 | CD1 | PHE | D | 119 | 62.953 | −2.236 | −33.030 | 1.00 | 34.79 | D |
| ATOM | 11748 | CD2 | PHE | D | 119 | 63.940 | −4.381 | −32.686 | 1.00 | 35.28 | D |
| ATOM | 11749 | CE1 | PHE | D | 119 | 61.774 | −2.684 | −32.410 | 1.00 | 35.86 | D |
| ATOM | 11750 | CE2 | PHE | D | 119 | 62.776 | −4.842 | −32.067 | 1.00 | 36.09 | D |
| ATOM | 11751 | CZ | PHE | D | 119 | 61.690 | −3.996 | −31.927 | 1.00 | 35.18 | D |
| ATOM | 11752 | C | PHE | D | 119 | 67.561 | −1.594 | −33.522 | 1.00 | 37.56 | D |
| ATOM | 11753 | O | PHE | D | 119 | 67.783 | −0.527 | −34.112 | 1.00 | 33.66 | D |
| ATOM | 11754 | N | GLN | D | 120 | 68.445 | −2.589 | −33.454 | 1.00 | 42.38 | D |
| ATOM | 11755 | CA | GLN | D | 120 | 69.754 | −2.543 | −34.102 | 1.00 | 45.75 | D |
| ATOM | 11756 | CB | GLN | D | 120 | 70.722 | −3.497 | −33.407 | 1.00 | 68.90 | D |
| ATOM | 11757 | CG | GLN | D | 120 | 71.977 | −2.863 | −32.846 | 1.00 | 69.05 | D |
| ATOM | 11758 | CD | GLN | D | 120 | 72.741 | −2.072 | −33.879 | 1.00 | 68.65 | D |
| ATOM | 11759 | OE1 | GLN | D | 120 | 72.877 | −2.502 | −35.021 | 1.00 | 68.21 | D |
| ATOM | 11760 | NE2 | GLN | D | 120 | 73.260 | −0.912 | −33.482 | 1.00 | 71.48 | D |
| ATOM | 11761 | C | GLN | D | 120 | 69.501 | −3.050 | −35.513 | 1.00 | 44.89 | D |
| ATOM | 11762 | O | GLN | D | 120 | 69.021 | −4.172 | −35.674 | 1.00 | 47.59 | D |
| ATOM | 11763 | N | LEU | D | 121 | 69.811 | −2.238 | −36.522 | 1.00 | 32.65 | D |
| ATOM | 11764 | CA | LEU | D | 121 | 69.597 | −2.622 | −37.916 | 1.00 | 34.31 | D |
| ATOM | 11765 | CB | LEU | D | 121 | 68.544 | −1.711 | −38.546 | 1.00 | 31.99 | D |
| ATOM | 11766 | CG | LEU | D | 121 | 67.068 | −2.140 | −38.483 | 1.00 | 30.26 | D |
| ATOM | 11767 | CD1 | LEU | D | 121 | 66.778 | −2.964 | −37.261 | 1.00 | 31.65 | D |
| ATOM | 11768 | CD2 | LEU | D | 121 | 66.186 | −0.893 | −38.505 | 1.00 | 25.75 | D |
| ATOM | 11769 | C | LEU | D | 121 | 70.866 | −2.579 | −38.762 | 1.00 | 36.35 | D |
| ATOM | 11770 | O | LEU | D | 121 | 71.758 | −1.776 | −38.492 | 1.00 | 41.62 | D |
| ATOM | 11771 | N | GLU | D | 122 | 70.934 | −3.446 | −39.779 | 1.00 | 48.11 | D |
| ATOM | 11772 | CA | GLU | D | 122 | 72.071 | −3.533 | −40.705 | 1.00 | 49.65 | D |
| ATOM | 11773 | CB | GLU | D | 122 | 72.319 | −4.971 | −41.174 | 1.00 | 104.93 | D |
| ATOM | 11774 | CG | GLU | D | 122 | 72.642 | −6.018 | −40.135 | 1.00 | 107.97 | D |
| ATOM | 11775 | CD | GLU | D | 122 | 72.742 | −7.416 | −40.765 | 1.00 | 113.25 | D |
| ATOM | 11776 | OE1 | GLU | D | 122 | 71.730 | −7.906 | −41.318 | 1.00 | 114.75 | D |
| ATOM | 11777 | OE2 | GLU | D | 122 | 73.835 | −8.023 | −40.717 | 1.00 | 113.34 | D |
| ATOM | 11778 | C | GLU | D | 122 | 71.755 | −2.739 | −41.967 | 1.00 | 48.45 | D |
| ATOM | 11779 | O | GLU | D | 122 | 70.601 | −2.615 | −42.363 | 1.00 | 49.64 | D |
| ATOM | 11780 | N | LYS | D | 123 | 72.783 | −2.220 | −42.619 | 1.00 | 54.35 | D |
| ATOM | 11781 | CA | LYS | D | 123 | 72.560 | −1.505 | −43.865 | 1.00 | 55.20 | D |
| ATOM | 11782 | CB | LYS | D | 123 | 73.883 | −1.010 | −44.446 | 1.00 | 62.54 | D |
| ATOM | 11783 | CG | LYS | D | 123 | 73.751 | −0.457 | −45.840 | 1.00 | 58.70 | D |
| ATOM | 11784 | CD | LYS | D | 123 | 75.093 | −0.049 | −46.395 | 1.00 | 56.37 | D |
| ATOM | 11785 | CE | LYS | D | 123 | 74.950 | 0.434 | −47.827 | 1.00 | 58.10 | D |
| ATOM | 11786 | NZ | LYS | D | 123 | 76.273 | 0.713 | −48.462 | 1.00 | 62.11 | D |
| ATOM | 11787 | C | LYS | D | 123 | 71.925 | −2.511 | −44.825 | 1.00 | 56.02 | D |
| ATOM | 11788 | O | LYS | D | 123 | 72.448 | −3.606 | −45.025 | 1.00 | 60.78 | D |
| ATOM | 11789 | N | GLY | D | 124 | 70.783 | −2.149 | −45.393 | 1.00 | 39.30 | D |
| ATOM | 11790 | CA | GLY | D | 124 | 70.120 | −3.041 | −46.323 | 1.00 | 40.41 | D |
| ATOM | 11791 | C | GLY | D | 124 | 68.888 | −3.745 | −45.785 | 1.00 | 41.82 | D |
| ATOM | 11792 | O | GLY | D | 124 | 68.132 | −4.335 | −46.555 | 1.00 | 42.62 | D |
| ATOM | 11793 | N | ASP | D | 125 | 68.673 | −3.700 | −44.473 | 1.00 | 47.24 | D |
| ATOM | 11794 | CA | ASP | D | 125 | 67.505 | −4.358 | −43.895 | 1.00 | 46.03 | D |
| ATOM | 11795 | CB | ASP | D | 125 | 67.512 | −4.227 | −42.373 | 1.00 | 68.14 | D |
| ATOM | 11796 | CG | ASP | D | 125 | 68.592 | −5.064 | −41.714 | 1.00 | 73.60 | D |
| ATOM | 11797 | OD1 | ASP | D | 125 | 69.223 | −5.895 | −42.403 | 1.00 | 75.25 | D |
| ATOM | 11798 | OD2 | ASP | D | 125 | 68.796 | −4.893 | −40.495 | 1.00 | 72.26 | D |
| ATOM | 11799 | C | ASP | D | 125 | 66.194 | −3.787 | −44.442 | 1.00 | 45.08 | D |
| ATOM | 11800 | O | ASP | D | 125 | 66.105 | −2.599 | −44.771 | 1.00 | 41.99 | D |
| ATOM | 11801 | N | ARG | D | 126 | 65.183 | −4.645 | −44.533 | 1.00 | 56.90 | D |
| ATOM | 11802 | CA | ARG | D | 126 | 63.870 | −4.258 | −45.035 | 1.00 | 52.38 | D |
| ATOM | 11803 | CB | ARG | D | 126 | 63.441 | −5.190 | −46.171 | 1.00 | 74.96 | D |
| ATOM | 11804 | CG | ARG | D | 126 | 64.223 | −5.065 | −47.451 | 1.00 | 73.45 | D |
| ATOM | 11805 | CD | ARG | D | 126 | 63.856 | −6.199 | −48.415 | 1.00 | 78.37 | D |
| ATOM | 11806 | NE | ARG | D | 126 | 64.301 | −5.921 | −49.782 | 1.00 | 81.92 | D |
| ATOM | 11807 | CZ | ARG | D | 126 | 63.681 | −5.095 | −50.634 | 1.00 | 85.15 | D |
| ATOM | 11808 | NH1 | ARG | D | 126 | 64.168 | −4.897 | −51.858 | 1.00 | 84.82 | D |
| ATOM | 11809 | NH2 | ARG | D | 126 | 62.559 | −4.479 | −50.276 | 1.00 | 83.34 | D |
| ATOM | 11810 | C | ARG | D | 126 | 62.834 | −4.345 | −43.916 | 1.00 | 53.22 | D |
| ATOM | 11811 | O | ARG | D | 126 | 62.668 | −5.390 | −43.295 | 1.00 | 50.44 | D |
| ATOM | 11812 | N | LEU | D | 127 | 62.124 | −3.254 | −43.666 | 1.00 | 49.34 | D |
| ATOM | 11813 | CA | LEU | D | 127 | 61.102 | −3.243 | −42.627 | 1.00 | 45.72 | D |
| ATOM | 11814 | CB | LEU | D | 127 | 61.290 | −2.021 | −41.737 | 1.00 | 39.55 | D |
| ATOM | 11815 | CG | LEU | D | 127 | 62.651 | −1.992 | −41.043 | 1.00 | 38.53 | D |
| ATOM | 11816 | CD1 | LEU | D | 127 | 62.862 | −0.617 | −40.435 | 1.00 | 39.11 | D |
| ATOM | 11817 | CD2 | LEU | D | 127 | 62.724 | −3.094 | −39.969 | 1.00 | 39.19 | D |
| ATOM | 11818 | C | LEU | D | 127 | 59.709 | −3.235 | −43.246 | 1.00 | 45.47 | D |
| ATOM | 11819 | O | LEU | D | 127 | 59.456 | −2.507 | −44.209 | 1.00 | 41.67 | D |
| ATOM | 11820 | N | SER | D | 128 | 58.810 | −4.050 | −42.696 | 1.00 | 49.43 | D |
| ATOM | 11821 | CA | SER | D | 128 | 57.443 | −4.143 | −43.200 | 1.00 | 49.10 | D |
| ATOM | 11822 | CB | SER | D | 128 | 57.160 | −5.544 | −43.722 | 1.00 | 56.89 | D |
| ATOM | 11823 | OG | SER | D | 128 | 58.273 | −6.055 | −44.420 | 1.00 | 62.74 | D |
| ATOM | 11824 | C | SER | D | 128 | 56.453 | −3.852 | −42.092 | 1.00 | 48.28 | D |
| ATOM | 11825 | O | SER | D | 128 | 56.656 | −4.257 | −40.953 | 1.00 | 49.56 | D |
| ATOM | 11826 | N | ALA | D | 129 | 55.384 | −3.142 | −42.434 | 1.00 | 40.32 | D |
| ATOM | 11827 | CA | ALA | D | 129 | 54.320 | −2.820 | −41.482 | 1.00 | 41.29 | D |
| ATOM | 11828 | CB | ALA | D | 129 | 54.225 | −1.299 | −41.267 | 1.00 | 25.63 | D |
| ATOM | 11829 | C | ALA | D | 129 | 53.057 | −3.357 | −42.144 | 1.00 | 40.06 | D |
| ATOM | 11830 | O | ALA | D | 129 | 52.479 | −2.699 | −43.010 | 1.00 | 40.94 | D |
| ATOM | 11831 | N | GLU | D | 130 | 52.639 | −4.555 | −41.748 | 1.00 | 49.08 | D |
| ATOM | 11832 | CA | GLU | D | 130 | 51.477 | −5.191 | −42.357 | 1.00 | 50.05 | D |
| ATOM | 11833 | CB | GLU | D | 130 | 51.921 | −6.499 | −43.004 | 1.00 | 97.81 | D |
| ATOM | 11834 | CG | GLU | D | 130 | 52.987 | −6.296 | −44.059 | 1.00 | 97.35 | D |
| ATOM | 11835 | CD | GLU | D | 130 | 53.787 | −7.552 | −44.354 | 1.00 | 102.84 | D |
| ATOM | 11836 | OE1 | GLU | D | 130 | 54.646 | −7.504 | −45.266 | 1.00 | 104.21 | D |
| ATOM | 11837 | OE2 | GLU | D | 130 | 53.567 | −8.581 | −43.676 | 1.00 | 102.65 | D |
| ATOM | 11838 | C | GLU | D | 130 | 50.318 | −5.455 | −41.416 | 1.00 | 46.44 | D |
| ATOM | 11839 | O | GLU | D | 130 | 50.482 | −5.433 | −40.198 | 1.00 | 46.36 | D |
| ATOM | 11840 | N | ILE | D | 131 | 49.142 | −5.688 | −41.995 | 1.00 | 61.82 | D |
| ATOM | 11841 | CA | ILE | D | 131 | 47.924 | −5.999 | −41.237 | 1.00 | 64.36 | D |
| ATOM | 11842 | CB | ILE | D | 131 | 46.902 | −4.846 | −41.256 | 1.00 | 50.40 | D |
| ATOM | 11843 | CG2 | ILE | D | 131 | 47.455 | −3.650 | −40.504 | 1.00 | 50.63 | D |
| ATOM | 11844 | CG1 | ILE | D | 131 | 46.551 | −4.497 | −42.706 | 1.00 | 46.59 | D |
| ATOM | 11845 | CD1 | ILE | D | 131 | 45.529 | −3.382 | −42.840 | 1.00 | 45.20 | D |
| ATOM | 11846 | C | ILE | D | 131 | 47.285 | −7.196 | −41.914 | 1.00 | 66.25 | D |
| ATOM | 11847 | O | ILE | D | 131 | 47.598 | −7.502 | −43.058 | 1.00 | 66.97 | D |
| ATOM | 11848 | N | ASN | D | 132 | 46.376 | −7.863 | −41.219 | 1.00 | 63.98 | D |
| ATOM | 11849 | CA | ASN | D | 132 | 45.723 | −9.041 | −41.773 | 1.00 | 60.98 | D |
| ATOM | 11850 | CB | ASN | D | 132 | 45.540 | −10.077 | −40.670 | 1.00 | 56.42 | D |
| ATOM | 11851 | CG | ASN | D | 132 | 44.568 | −9.618 | −39.610 | 1.00 | 55.12 | D |
| ATOM | 11852 | OD1 | ASN | D | 132 | 44.566 | −8.458 | −39.203 | 1.00 | 56.81 | D |
| ATOM | 11853 | ND2 | ASN | D | 132 | 43.735 | −10.531 | −39.148 | 1.00 | 52.95 | D |
| ATOM | 11854 | C | ASN | D | 132 | 44.376 | −8.760 | −42.445 | 1.00 | 58.63 | D |
| ATOM | 11855 | O | ASN | D | 132 | 43.976 | −9.482 | −43.353 | 1.00 | 61.80 | D |
| ATOM | 11856 | N | ARG | D | 133 | 43.677 | −7.716 | −42.007 | 1.00 | 67.78 | D |
| ATOM | 11857 | CA | ARG | D | 133 | 42.371 | −7.382 | −42.575 | 1.00 | 63.37 | D |
| ATOM | 11858 | CB | ARG | D | 133 | 41.289 | −7.476 | −41.497 | 1.00 | 93.15 | D |
| ATOM | 11859 | CG | ARG | D | 133 | 41.122 | −8.861 | −40.906 | 1.00 | 95.73 | D |
| ATOM | 11860 | CD | ARG | D | 133 | 40.810 | −9.890 | −41.987 | 1.00 | 100.97 | D |
| ATOM | 11861 | NE | ARG | D | 133 | 39.584 | −9.576 | −42.719 | 1.00 | 98.83 | D |
| ATOM | 11862 | CZ | ARG | D | 133 | 38.356 | −9.677 | −42.213 | 1.00 | 99.93 | D |
| ATOM | 11863 | NH1 | ARG | D | 133 | 37.303 | −9.366 | −42.956 | 1.00 | 102.61 | D |
| ATOM | 11864 | NH2 | ARG | D | 133 | 38.179 | −10.098 | −40.968 | 1.00 | 99.22 | D |
| ATOM | 11865 | C | ARG | D | 133 | 42.319 | −5.996 | −43.212 | 1.00 | 67.96 | D |
| ATOM | 11866 | O | ARG | D | 133 | 41.629 | −5.100 | −42.717 | 1.00 | 70.32 | D |
| ATOM | 11867 | N | PRO | D | 134 | 43.031 | −5.809 | −44.339 | 1.00 | 59.73 | D |
| ATOM | 11868 | CD | PRO | D | 134 | 43.766 | −6.829 | −45.111 | 1.00 | 50.11 | D |
| ATOM | 11869 | CA | PRO | D | 134 | 43.059 | −4.520 | −45.037 | 1.00 | 63.45 | D |
| ATOM | 11870 | CB | PRO | D | 134 | 43.985 | −4.786 | −46.216 | 1.00 | 52.83 | D |
| ATOM | 11871 | CG | PRO | D | 134 | 43.763 | −6.231 | −46.495 | 1.00 | 54.83 | D |
| ATOM | 11872 | C | PRO | D | 134 | 41.693 | −4.036 | −45.470 | 1.00 | 68.63 | D |
| ATOM | 11873 | O | PRO | D | 134 | 41.536 | −2.884 | −45.868 | 1.00 | 71.67 | D |
| ATOM | 11874 | N | ASP | D | 135 | 40.703 | −4.916 | −45.394 | 1.00 | 71.22 | D |
| ATOM | 11875 | CA | ASP | D | 135 | 39.346 | −4.544 | −45.774 | 1.00 | 67.68 | D |
| ATOM | 11876 | CB | ASP | D | 135 | 38.528 | −5.798 | −46.085 | 1.00 | 108.15 | D |
| ATOM | 11877 | CG | ASP | D | 135 | 38.760 | −6.905 | −45.087 | 1.00 | 107.62 | D |
| ATOM | 11878 | OD1 | ASP | D | 135 | 38.245 | −6.800 | −43.957 | 1.00 | 103.56 | D |
| ATOM | 11879 | OD2 | ASP | D | 135 | 39.466 | −7.878 | −45.425 | 1.00 | 109.20 | D |
| ATOM | 11880 | C | ASP | D | 135 | 38.666 | −3.700 | −44.691 | 1.00 | 66.39 | D |
| ATOM | 11881 | O | ASP | D | 135 | 37.613 | −3.108 | −44.929 | 1.00 | 73.08 | D |
| ATOM | 11882 | N | TYR | D | 136 | 39.283 | −3.640 | −43.510 | 1.00 | 67.98 | D |
| ATOM | 11883 | CA | TYR | D | 136 | 38.756 | −2.857 | −42.393 | 1.00 | 69.19 | D |
| ATOM | 11884 | CB | TYR | D | 136 | 38.765 | −3.680 | −41.108 | 1.00 | 76.23 | D |
| ATOM | 11885 | CG | TYR | D | 136 | 37.689 | −4.734 | −41.015 | 1.00 | 75.84 | D |
| ATOM | 11886 | CD1 | TYR | D | 136 | 38.022 | −6.076 | −40.851 | 1.00 | 75.44 | D |
| ATOM | 11887 | CE1 | TYR | D | 136 | 37.042 | −7.046 | −40.706 | 1.00 | 77.99 | D |
| ATOM | 11888 | CD2 | TYR | D | 136 | 36.339 | −4.388 | −41.038 | 1.00 | 78.01 | D |
| ATOM | 11889 | CE2 | TYR | D | 136 | 35.348 | −5.354 | −40.895 | 1.00 | 77.48 | D |
| ATOM | 11890 | CZ | TYR | D | 136 | 35.709 | −6.681 | −40.728 | 1.00 | 78.78 | D |
| ATOM | 11891 | OH | TYR | D | 136 | 34.741 | −7.647 | −40.571 | 1.00 | 82.62 | D |
| ATOM | 11892 | C | TYR | D | 136 | 39.596 | −1.603 | −42.163 | 1.00 | 71.10 | D |
| ATOM | 11893 | O | TYR | D | 136 | 39.384 | −0.869 | −41.199 | 1.00 | 73.98 | D |
| ATOM | 11894 | N | LEU | D | 137 | 40.551 | −1.365 | −43.050 | 1.00 | 65.14 | D |
| ATOM | 11895 | CA | LEU | D | 137 | 41.433 | −0.214 | −42.930 | 1.00 | 61.85 | D |
| ATOM | 11896 | CB | LEU | D | 137 | 42.542 | −0.301 | −43.977 | 1.00 | 51.37 | D |
| ATOM | 11897 | CG | LEU | D | 137 | 43.979 | −0.362 | −43.470 | 1.00 | 49.10 | D |
| ATOM | 11898 | CD1 | LEU | D | 137 | 44.915 | −0.184 | −44.649 | 1.00 | 51.24 | D |
| ATOM | 11899 | CD2 | LEU | D | 137 | 44.221 | 0.727 | −42.436 | 1.00 | 52.86 | D |
| ATOM | 11900 | C | LEU | D | 137 | 40.735 | 1.127 | −43.086 | 1.00 | 61.22 | D |
| ATOM | 11901 | O | LEU | D | 137 | 40.195 | 1.415 | −44.151 | 1.00 | 60.07 | D |
| ATOM | 11902 | N | LEU | D | 138 | 40.747 | 1.948 | −42.034 | 1.00 | 60.14 | D |
| ATOM | 11903 | CA | LEU | D | 138 | 40.144 | 3.279 | −42.119 | 1.00 | 57.94 | D |
| ATOM | 11904 | CB | LEU | D | 138 | 39.338 | 3.649 | −40.863 | 1.00 | 63.88 | D |
| ATOM | 11905 | CG | LEU | D | 138 | 38.948 | 5.146 | −40.843 | 1.00 | 60.00 | D |
| ATOM | 11906 | CD1 | LEU | D | 138 | 38.350 | 5.543 | −42.183 | 1.00 | 61.57 | D |
| ATOM | 11907 | CD2 | LEU | D | 138 | 37.967 | 5.440 | −39.725 | 1.00 | 62.92 | D |
| ATOM | 11908 | C | LEU | D | 138 | 41.206 | 4.346 | −42.333 | 1.00 | 61.29 | D |
| ATOM | 11909 | O | LEU | D | 138 | 41.826 | 4.827 | −41.387 | 1.00 | 60.44 | D |
| ATOM | 11910 | N | PHE | D | 139 | 41.411 | 4.714 | −43.588 | 1.00 | 67.96 | D |
| ATOM | 11911 | CA | PHE | D | 139 | 42.383 | 5.738 | −43.916 | 1.00 | 69.34 | D |
| ATOM | 11912 | CB | PHE | D | 139 | 43.457 | 5.170 | −44.851 | 1.00 | 74.23 | D |
| ATOM | 11913 | CG | PHE | D | 139 | 42.916 | 4.327 | −45.974 | 1.00 | 73.38 | D |
| ATOM | 11914 | CD1 | PHE | D | 139 | 43.687 | 3.300 | −46.513 | 1.00 | 72.91 | D |
| ATOM | 11915 | CD2 | PHE | D | 139 | 41.655 | 4.565 | −46.512 | 1.00 | 75.99 | D |
| ATOM | 11916 | CE1 | PHE | D | 139 | 43.212 | 2.526 | −47.565 | 1.00 | 77.55 | D |
| ATOM | 11917 | CE2 | PHE | D | 139 | 41.175 | 3.795 | −47.563 | 1.00 | 73.53 | D |
| ATOM | 11918 | CZ | PHE | D | 139 | 41.957 | 2.775 | −48.089 | 1.00 | 77.47 | D |
| ATOM | 11919 | C | PHE | D | 139 | 41.653 | 6.904 | −44.559 | 1.00 | 73.37 | D |
| ATOM | 11920 | O | PHE | D | 139 | 42.138 | 7.511 | −45.510 | 1.00 | 73.47 | D |
| ATOM | 11921 | N | ALA | D | 140 | 40.477 | 7.209 | −44.014 | 1.00 | 78.45 | D |
| ATOM | 11922 | CA | ALA | D | 140 | 39.638 | 8.292 | −44.518 | 1.00 | 79.75 | D |
| ATOM | 11923 | CB | ALA | D | 140 | 38.194 | 8.072 | −44.100 | 1.00 | 66.14 | D |
| ATOM | 11924 | C | ALA | D | 140 | 40.088 | 9.661 | −44.051 | 1.00 | 80.14 | D |
| ATOM | 11925 | O | ALA | D | 140 | 40.151 | 10.597 | −44.843 | 1.00 | 82.03 | D |
| ATOM | 11926 | N | GLU | D | 141 | 40.397 | 9.780 | −42.764 | 1.00 | 109.92 | D |
| ATOM | 11927 | CA | GLU | D | 141 | 40.813 | 11.059 | −42.211 | 1.00 | 113.10 | D |
| ATOM | 11928 | CB | GLU | D | 141 | 40.768 | 11.025 | −40.678 | 1.00 | 200.84 | D |
| ATOM | 11929 | CG | GLU | D | 141 | 41.047 | 12.374 | −40.014 | 1.00 | 200.84 | D |
| ATOM | 11930 | CD | GLU | D | 141 | 42.531 | 12.660 | −39.832 | 1.00 | 200.84 | D |
| ATOM | 11931 | OE1 | GLU | D | 141 | 42.906 | 13.849 | −39.765 | 1.00 | 200.84 | D |
| ATOM | 11932 | OE2 | GLU | D | 141 | 43.323 | 11.700 | −39.740 | 1.00 | 200.84 | D |
| ATOM | 11933 | C | GLU | D | 141 | 42.182 | 11.525 | −42.677 | 1.00 | 115.20 | D |
| ATOM | 11934 | O | GLU | D | 141 | 42.276 | 12.327 | −43.603 | 1.00 | 118.88 | D |
| ATOM | 11935 | N | SER | D | 142 | 43.246 | 11.028 | −42.058 | 1.00 | 82.32 | D |
| ATOM | 11936 | CA | SER | D | 142 | 44.572 | 11.488 | −42.445 | 1.00 | 85.31 | D |
| ATOM | 11937 | CB | SER | D | 142 | 44.625 | 13.005 | −42.306 | 1.00 | 58.12 | D |
| ATOM | 11938 | OG | SER | D | 142 | 45.739 | 13.534 | −42.985 | 1.00 | 59.00 | D |
| ATOM | 11939 | C | SER | D | 142 | 45.725 | 10.873 | −41.644 | 1.00 | 86.63 | D |
| ATOM | 11940 | O | SER | D | 142 | 46.428 | 9.966 | −42.122 | 1.00 | 91.72 | D |
| ATOM | 11941 | N | GLY | D | 143 | 45.938 | 11.384 | −40.436 | 1.00 | 48.82 | D |
| ATOM | 11942 | CA | GLY | D | 143 | 47.008 | 10.846 | −39.621 | 1.00 | 46.29 | D |
| ATOM | 11943 | C | GLY | D | 143 | 46.556 | 9.632 | −38.837 | 1.00 | 44.36 | D |
| ATOM | 11944 | O | GLY | D | 143 | 46.973 | 9.470 | −37.691 | 1.00 | 46.35 | D |
| ATOM | 11945 | N | GLN | D | 144 | 45.723 | 8.782 | −39.451 | 1.00 | 51.06 | D |
| ATOM | 11946 | CA | GLN | D | 144 | 45.185 | 7.575 | −38.811 | 1.00 | 45.59 | D |
| ATOM | 11947 | CB | GLN | D | 144 | 43.739 | 7.342 | −39.249 | 1.00 | 74.25 | D |
| ATOM | 11948 | CG | GLN | D | 144 | 42.872 | 8.582 | −39.199 | 1.00 | 73.53 | D |
| ATOM | 11949 | CD | GLN | D | 144 | 41.418 | 8.286 | −39.499 | 1.00 | 73.50 | D |
| ATOM | 11950 | OE1 | GLN | D | 144 | 41.096 | 7.612 | −40.481 | 1.00 | 79.07 | D |
| ATOM | 11951 | NE2 | GLN | D | 144 | 40.525 | 8.801 | −38.658 | 1.00 | 72.04 | D |
| ATOM | 11952 | C | GLN | D | 144 | 45.988 | 6.310 | −39.103 | 1.00 | 47.39 | D |
| ATOM | 11953 | O | GLN | D | 144 | 45.927 | 5.345 | −38.345 | 1.00 | 49.70 | D |
| ATOM | 11954 | N | VAL | D | 145 | 46.735 | 6.313 | −40.204 | 1.00 | 47.02 | D |
| ATOM | 11955 | CA | VAL | D | 145 | 47.543 | 5.159 | −40.592 | 1.00 | 45.27 | D |
| ATOM | 11956 | CB | VAL | D | 145 | 46.855 | 4.381 | −41.728 | 1.00 | 32.65 | D |
| ATOM | 11957 | CG1 | VAL | D | 145 | 47.651 | 3.129 | −42.092 | 1.00 | 30.81 | D |
| ATOM | 11958 | CG2 | VAL | D | 145 | 45.468 | 4.002 | −41.298 | 1.00 | 29.73 | D |
| ATOM | 11959 | C | VAL | D | 145 | 48.921 | 5.623 | −41.058 | 1.00 | 46.38 | D |
| ATOM | 11960 | O | VAL | D | 145 | 49.044 | 6.293 | −42.081 | 1.00 | 49.06 | D |
| ATOM | 11961 | N | TYR | D | 146 | 49.953 | 5.269 | −40.301 | 1.00 | 48.48 | D |
| ATOM | 11962 | CA | TYR | D | 146 | 51.311 | 5.669 | −40.641 | 1.00 | 46.69 | D |
| ATOM | 11963 | CB | TYR | D | 146 | 51.663 | 7.032 | −40.013 | 1.00 | 44.35 | D |
| ATOM | 11964 | CG | TYR | D | 146 | 51.328 | 7.156 | −38.541 | 1.00 | 44.38 | D |
| ATOM | 11965 | CD1 | TYR | D | 146 | 50.046 | 7.516 | −38.118 | 1.00 | 43.77 | D |
| ATOM | 11966 | CE1 | TYR | D | 146 | 49.722 | 7.589 | −36.765 | 1.00 | 42.56 | D |
| ATOM | 11967 | CD2 | TYR | D | 146 | 52.281 | 6.875 | −37.568 | 1.00 | 39.97 | D |
| ATOM | 11968 | CE2 | TYR | D | 146 | 51.971 | 6.943 | −36.202 | 1.00 | 42.02 | D |
| ATOM | 11969 | CZ | TYR | D | 146 | 50.693 | 7.300 | −35.807 | 1.00 | 42.06 | D |
| ATOM | 11970 | OH | TYR | D | 146 | 50.402 | 7.358 | −34.459 | 1.00 | 43.04 | D |
| ATOM | 11971 | C | TYR | D | 146 | 52.337 | 4.632 | −40.214 | 1.00 | 45.00 | D |
| ATOM | 11972 | O | TYR | D | 146 | 52.063 | 3.725 | −39.425 | 1.00 | 43.17 | D |
| ATOM | 11973 | N | PHE | D | 147 | 53.539 | 4.801 | −40.733 | 1.00 | 36.89 | D |
| ATOM | 11974 | CA | PHE | D | 147 | 54.641 | 3.890 | −40.489 | 1.00 | 36.41 | D |
| ATOM | 11975 | CB | PHE | D | 147 | 54.538 | 2.761 | −41.522 | 1.00 | 33.59 | D |
| ATOM | 11976 | CG | PHE | D | 147 | 55.730 | 1.853 | −41.583 | 1.00 | 32.64 | D |
| ATOM | 11977 | CD1 | PHE | D | 147 | 56.427 | 1.496 | −40.427 | 1.00 | 33.13 | D |
| ATOM | 11978 | CD2 | PHE | D | 147 | 56.108 | 1.280 | −42.808 | 1.00 | 34.97 | D |
| ATOM | 11979 | CE1 | PHE | D | 147 | 57.479 | 0.573 | −40.491 | 1.00 | 33.46 | D |
| ATOM | 11980 | CE2 | PHE | D | 147 | 57.150 | 0.360 | −42.878 | 1.00 | 33.54 | D |
| ATOM | 11981 | CZ | PHE | D | 147 | 57.839 | 0.002 | −41.717 | 1.00 | 34.38 | D |
| ATOM | 11982 | C | PHE | D | 147 | 55.908 | 4.719 | −40.674 | 1.00 | 37.95 | D |
| ATOM | 11983 | O | PHE | D | 147 | 56.067 | 5.418 | −41.676 | 1.00 | 35.43 | D |
| ATOM | 11984 | N | GLY | D | 148 | 56.802 | 4.665 | −39.702 | 1.00 | 26.26 | D |
| ATOM | 11985 | CA | GLY | D | 148 | 58.017 | 5.447 | −39.821 | 1.00 | 30.74 | D |
| ATOM | 11986 | C | GLY | D | 148 | 59.186 | 4.944 | −39.006 | 1.00 | 32.96 | D |
| ATOM | 11987 | O | GLY | D | 148 | 59.039 | 4.065 | −38.150 | 1.00 | 35.57 | D |
| ATOM | 11988 | N | ILE | D | 149 | 60.357 | 5.496 | −39.294 | 1.00 | 38.11 | D |
| ATOM | 11989 | CA | ILE | D | 149 | 61.579 | 5.135 | −38.592 | 1.00 | 36.52 | D |
| ATOM | 11990 | CB | ILE | D | 149 | 62.301 | 3.925 | −39.238 | 1.00 | 41.08 | D |
| ATOM | 11991 | CG2 | ILE | D | 149 | 61.338 | 2.788 | −39.475 | 1.00 | 41.63 | D |
| ATOM | 11992 | CG1 | ILE | D | 149 | 62.889 | 4.315 | −40.578 | 1.00 | 37.12 | D |
| ATOM | 11993 | CD1 | ILE | D | 149 | 63.715 | 3.212 | −41.149 | 1.00 | 40.37 | D |
| ATOM | 11994 | C | ILE | D | 149 | 62.527 | 6.322 | −38.612 | 1.00 | 38.56 | D |
| ATOM | 11995 | O | ILE | D | 149 | 62.553 | 7.111 | −39.555 | 1.00 | 43.73 | D |
| ATOM | 11996 | N | ILE | D | 150 | 63.315 | 6.435 | −37.559 | 1.00 | 38.83 | D |
| ATOM | 11997 | CA | ILE | D | 150 | 64.275 | 7.521 | −37.421 | 1.00 | 39.34 | D |
| ATOM | 11998 | CB | ILE | D | 150 | 63.707 | 8.635 | −36.484 | 1.00 | 41.58 | D |
| ATOM | 11999 | CG2 | ILE | D | 150 | 63.294 | 8.049 | −35.141 | 1.00 | 44.88 | D |
| ATOM | 12000 | CG1 | ILE | D | 150 | 64.745 | 9.723 | −36.258 | 1.00 | 39.57 | D |
| ATOM | 12001 | CD1 | ILE | D | 150 | 64.987 | 10.579 | −37.457 | 1.00 | 37.58 | D |
| ATOM | 12002 | C | ILE | D | 150 | 65.543 | 6.915 | −36.826 | 1.00 | 40.68 | D |
| ATOM | 12003 | O | ILE | D | 150 | 65.490 | 6.109 | −35.901 | 1.00 | 39.79 | D |
| ATOM | 12004 | N | ALA | D | 151 | 66.687 | 7.288 | −37.373 | 1.00 | 52.91 | D |
| ATOM | 12005 | CA | ALA | D | 151 | 67.959 | 6.777 | −36.876 | 1.00 | 51.65 | D |
| ATOM | 12006 | CB | ALA | D | 151 | 69.031 | 6.933 | −37.943 | 1.00 | 21.30 | D |
| ATOM | 12007 | C | ALA | D | 151 | 68.372 | 7.531 | −35.619 | 1.00 | 52.61 | D |
| ATOM | 12008 | O | ALA | D | 151 | 68.497 | 8.751 | −35.635 | 1.00 | 54.99 | D |
| ATOM | 12009 | N | LEU | D | 152 | 68.589 | 6.804 | −34.533 | 1.00 | 73.88 | D |
| ATOM | 12010 | CA | LEU | D | 152 | 68.993 | 7.429 | −33.287 | 1.00 | 77.86 | D |
| ATOM | 12011 | CB | LEU | D | 152 | 68.191 | 6.853 | −32.129 | 1.00 | 65.80 | D |
| ATOM | 12012 | CG | LEU | D | 152 | 66.687 | 7.075 | −32.250 | 1.00 | 67.05 | D |
| ATOM | 12013 | CD1 | LEU | D | 152 | 65.972 | 6.430 | −31.078 | 1.00 | 64.74 | D |
| ATOM | 12014 | CD2 | LEU | D | 152 | 66.398 | 8.557 | −32.298 | 1.00 | 66.25 | D |
| ATOM | 12015 | C | LEU | D | 152 | 70.478 | 7.218 | −33.041 | 1.00 | 80.71 | D |
| ATOM | 12016 | O | LEU | D | 152 | 71.267 | 8.161 | −33.116 | 1.00 | 86.57 | D |
| ATOM | 12017 | N | ILE | D | 153 | 48.240 | 13.732 | −61.027 | 1.00 | 42.86 | D |
| ATOM | 12018 | CA | ILE | D | 153 | 47.268 | 14.807 | −60.907 | 1.00 | 42.86 | D |
| ATOM | 12019 | CB | ILE | D | 153 | 46.347 | 14.600 | −59.700 | 1.00 | 34.19 | D |
| ATOM | 12020 | CG2 | ILE | D | 153 | 45.433 | 13.426 | −59.952 | 1.00 | 30.97 | D |
| ATOM | 12021 | CG1 | ILE | D | 153 | 47.177 | 14.380 | −58.437 | 1.00 | 30.97 | D |
| ATOM | 12022 | CD1 | ILE | D | 153 | 46.333 | 14.185 | −57.190 | 1.00 | 30.97 | D |
| ATOM | 12023 | C | ILE | D | 153 | 47.942 | 16.162 | −60.783 | 1.00 | 42.86 | D |
| ATOM | 12024 | O | ILE | D | 153 | 49.101 | 16.268 | −60.380 | 1.00 | 42.86 | D |
| ATOM | 12025 | N | THR | D | 154 | 47.179 | 17.199 | −61.108 | 1.00 | 43.30 | D |
| ATOM | 12026 | CA | THR | D | 154 | 47.672 | 18.569 | −61.107 | 1.00 | 43.30 | D |
| ATOM | 12027 | CB | THR | D | 154 | 48.093 | 18.935 | −62.536 | 1.00 | 47.08 | D |
| ATOM | 12028 | OG1 | THR | D | 154 | 49.517 | 18.888 | −62.622 | 1.00 | 57.36 | D |
| ATOM | 12029 | CG2 | THR | D | 154 | 47.567 | 20.311 | −62.949 | 1.00 | 57.36 | D |
| ATOM | 12030 | C | THR | D | 154 | 46.680 | 19.610 | −60.621 | 1.00 | 43.30 | D |
| ATOM | 12031 | O | THR | D | 154 | 45.488 | 19.496 | −60.868 | 1.00 | 43.30 | D |
| ATOM | 12032 | N | LEU | D | 155 | 47.181 | 20.625 | −59.927 | 1.00 | 32.69 | D |
| ATOM | 12033 | CA | LEU | D | 155 | 46.345 | 21.727 | −59.461 | 1.00 | 32.69 | D |
| ATOM | 12034 | CB | LEU | D | 155 | 46.115 | 21.656 | −57.942 | 1.00 | 35.97 | D |
| ATOM | 12035 | CG | LEU | D | 155 | 45.165 | 22.748 | −57.412 | 1.00 | 32.55 | D |
| ATOM | 12036 | CD1 | LEU | D | 155 | 43.856 | 22.717 | −58.172 | 1.00 | 32.55 | D |
| ATOM | 12037 | CD2 | LEU | D | 155 | 44.915 | 22.551 | −55.939 | 1.00 | 32.55 | D |
| ATOM | 12038 | C | LEU | D | 155 | 47.089 | 23.011 | −59.834 | 1.00 | 32.69 | D |
| ATOM | 12039 | O | LEU | D | 155 | 48.202 | 23.248 | −59.365 | 1.00 | 32.69 | D |
| ATOM | 12040 | N | LYS | D | 156 | 46.490 | 23.827 | −60.694 | 1.00 | 46.47 | D |
| ATOM | 12041 | CA | LYS | D | 156 | 47.137 | 25.061 | −61.128 | 1.00 | 46.47 | D |
| ATOM | 12042 | CB | LYS | D | 156 | 47.555 | 24.944 | −62.595 | 1.00 | 56.95 | D |
| ATOM | 12043 | CG | LYS | D | 156 | 48.331 | 26.159 | −63.083 | 1.00 | 59.14 | D |
| ATOM | 12044 | CD | LYS | D | 156 | 48.757 | 26.061 | −64.544 | 1.00 | 59.14 | D |
| ATOM | 12045 | CE | LYS | D | 156 | 47.584 | 26.184 | −65.509 | 1.00 | 59.14 | D |
| ATOM | 12046 | NZ | LYS | D | 156 | 48.032 | 26.202 | −66.939 | 1.00 | 59.14 | D |
| ATOM | 12047 | C | LYS | D | 156 | 46.289 | 26.321 | −60.960 | 1.00 | 46.47 | D |
| ATOM | 12048 | O | LYS | D | 156 | 45.131 | 26.352 | −61.354 | 1.00 | 46.47 | D |
| ATOM | 12049 | N | TYR | D | 157 | 46.882 | 27.357 | −60.376 | 1.00 | 45.11 | D |
| ATOM | 12050 | CA | TYR | D | 157 | 46.222 | 28.646 | −60.165 | 1.00 | 45.11 | D |
| ATOM | 12051 | CB | TYR | D | 157 | 46.320 | 29.069 | −58.695 | 1.00 | 43.96 | D |
| ATOM | 12052 | CG | TYR | D | 157 | 45.398 | 28.327 | −57.754 | 1.00 | 26.30 | D |
| ATOM | 12053 | CD1 | TYR | D | 157 | 44.605 | 27.267 | −58.201 | 1.00 | 26.30 | D |
| ATOM | 12054 | CE1 | TYR | D | 157 | 43.739 | 26.590 | −57.335 | 1.00 | 26.30 | D |
| ATOM | 12055 | CD2 | TYR | D | 157 | 45.305 | 28.692 | −56.413 | 1.00 | 26.30 | D |
| ATOM | 12056 | CE2 | TYR | D | 157 | 44.436 | 28.022 | −55.535 | 1.00 | 26.30 | D |
| ATOM | 12057 | CZ | TYR | D | 157 | 43.660 | 26.974 | −56.010 | 1.00 | 26.30 | D |
| ATOM | 12058 | OH | TYR | D | 157 | 42.817 | 26.311 | −55.153 | 1.00 | 26.30 | D |
| ATOM | 12059 | C | TYR | D | 157 | 46.935 | 29.679 | −61.051 | 1.00 | 45.11 | D |
| ATOM | 12060 | O | TYR | D | 157 | 48.173 | 29.804 | −61.019 | 1.00 | 45.11 | D |
| ATOM | 12061 | N | ASN | D | 158 | 46.156 | 30.417 | −61.840 | 1.00 | 38.49 | D |
| ATOM | 12062 | CA | ASN | D | 158 | 46.706 | 31.417 | −62.751 | 1.00 | 38.49 | D |
| ATOM | 12063 | CB | ASN | D | 158 | 46.529 | 30.934 | −64.208 | 1.00 | 30.54 | D |
| ATOM | 12064 | CG | ASN | D | 158 | 47.118 | 31.899 | −65.248 | 1.00 | 41.24 | D |
| ATOM | 12065 | OD1 | ASN | D | 158 | 47.613 | 32.971 | −64.916 | 1.00 | 41.24 | D |
| ATOM | 12066 | ND2 | ASN | D | 158 | 47.059 | 31.505 | −66.517 | 1.00 | 41.24 | D |
| ATOM | 12067 | C | ASN | D | 158 | 46.015 | 32.764 | −62.563 | 1.00 | 38.49 | D |
| ATOM | 12068 | O | ASN | D | 158 | 44.823 | 32.902 | −62.843 | 1.00 | 38.49 | D |
| ATOM | 12069 | N | TYR | D | 159 | 46.761 | 33.750 | −62.073 | 1.00 | 38.40 | D |
| ATOM | 12070 | CA | TYR | D | 159 | 46.215 | 35.092 | −61.899 | 1.00 | 38.40 | D |
| ATOM | 12071 | CB | TYR | D | 159 | 46.567 | 35.691 | −60.532 | 1.00 | 30.87 | D |
| ATOM | 12072 | CG | TYR | D | 159 | 46.014 | 34.979 | −59.327 | 1.00 | 34.43 | D |
| ATOM | 12073 | CD1 | TYR | D | 159 | 46.656 | 33.854 | −58.797 | 1.00 | 34.43 | D |
| ATOM | 12074 | CE1 | TYR | D | 159 | 46.186 | 33.226 | −57.648 | 1.00 | 34.43 | D |
| ATOM | 12075 | CD2 | TYR | D | 159 | 44.875 | 35.458 | −58.677 | 1.00 | 34.43 | D |
| ATOM | 12076 | CE2 | TYR | D | 159 | 44.390 | 34.836 | −57.513 | 1.00 | 34.43 | D |
| ATOM | 12077 | CZ | TYR | D | 159 | 45.057 | 33.721 | −57.001 | 1.00 | 34.43 | D |
| ATOM | 12078 | OH | TYR | D | 159 | 44.619 | 33.135 | −55.830 | 1.00 | 34.43 | D |
| ATOM | 12079 | C | TYR | D | 159 | 46.855 | 35.963 | −62.975 | 1.00 | 38.40 | D |
| ATOM | 12080 | O | TYR | D | 159 | 48.071 | 35.967 | −63.126 | 1.00 | 38.40 | D |
| ATOM | 12081 | N | THR | D | 160 | 46.050 | 36.708 | −63.720 | 1.00 | 48.26 | D |
| ATOM | 12082 | CA | THR | D | 160 | 46.617 | 37.560 | −64.751 | 1.00 | 48.26 | D |
| ATOM | 12083 | CB | THR | D | 160 | 46.580 | 36.843 | −66.110 | 1.00 | 59.02 | D |
| ATOM | 12084 | OG1 | THR | D | 160 | 47.219 | 35.568 | −65.986 | 1.00 | 46.36 | D |
| ATOM | 12085 | CG2 | THR | D | 160 | 47.293 | 37.651 | −67.162 | 1.00 | 46.36 | D |
| ATOM | 12086 | C | THR | D | 160 | 45.963 | 38.932 | −64.915 | 1.00 | 48.26 | D |
| ATOM | 12087 | O | THR | D | 160 | 44.740 | 39.036 | −65.050 | 1.00 | 48.26 | D |
| ATOM | 12088 | N | VAL | D | 161 | 46.776 | 39.987 | −64.868 | 1.00 | 38.08 | D |
| ATOM | 12089 | CA | VAL | D | 161 | 46.253 | 41.336 | −65.113 | 1.00 | 38.08 | D |
| ATOM | 12090 | CB | VAL | D | 161 | 46.573 | 42.336 | −63.991 | 1.00 | 53.65 | D |
| ATOM | 12091 | CG1 | VAL | D | 161 | 46.174 | 41.764 | −62.670 | 1.00 | 38.16 | D |
| ATOM | 12092 | CG2 | VAL | D | 161 | 48.028 | 42.682 | −64.001 | 1.00 | 38.16 | D |
| ATOM | 12093 | C | VAL | D | 161 | 46.905 | 41.801 | −66.418 | 1.00 | 38.08 | D |
| ATOM | 12094 | O | VAL | D | 161 | 48.099 | 41.596 | −66.634 | 1.00 | 38.08 | D |
| ATOM | 12095 | N | THR | D | 162 | 46.109 | 42.396 | −67.295 | 1.00 | 59.09 | D |
| ATOM | 12096 | CA | THR | D | 162 | 46.604 | 42.851 | −68.589 | 1.00 | 59.09 | D |
| ATOM | 12097 | CB | THR | D | 162 | 45.952 | 42.059 | −69.729 | 1.00 | 83.07 | D |
| ATOM | 12098 | OG1 | THR | D | 162 | 45.711 | 40.720 | −69.297 | 1.00 | 54.40 | D |
| ATOM | 12099 | CG2 | THR | D | 162 | 46.861 | 42.024 | −70.935 | 1.00 | 54.40 | D |
| ATOM | 12100 | C | THR | D | 162 | 46.291 | 44.324 | −68.811 | 1.00 | 59.09 | D |
| ATOM | 12101 | O | THR | D | 162 | 45.293 | 44.834 | −68.305 | 1.00 | 59.09 | D |
| ATOM | 12102 | N | LEU | D | 163 | 47.146 | 45.008 | −69.567 | 1.00 | 90.47 | D |
| ATOM | 12103 | CA | LEU | D | 163 | 46.918 | 46.413 | −69.857 | 1.00 | 90.47 | D |
| ATOM | 12104 | CB | LEU | D | 163 | 48.210 | 47.100 | −70.281 | 1.00 | 123.09 | D |
| ATOM | 12105 | CG | LEU | D | 163 | 48.544 | 48.333 | −69.437 | 1.00 | 71.18 | D |
| ATOM | 12106 | CD1 | LEU | D | 163 | 49.722 | 49.067 | −70.060 | 1.00 | 71.18 | D |
| ATOM | 12107 | CD2 | LEU | D | 163 | 47.346 | 49.253 | −69.346 | 1.00 | 71.18 | D |
| ATOM | 12108 | C | LEU | D | 163 | 45.893 | 46.527 | −70.975 | 1.00 | 90.47 | D |
| ATOM | 12109 | O | LEU | D | 163 | 45.427 | 47.619 | −71.294 | 1.00 | 90.47 | D |
| ATOM | 12110 | N | LYS | D | 164 | 45.533 | 45.391 | −71.562 | 1.00 | 184.19 | D |
| ATOM | 12111 | CA | LYS | D | 164 | 44.558 | 45.385 | −72.644 | 1.00 | 184.19 | D |
| ATOM | 12112 | CB | LYS | D | 164 | 43.153 | 45.649 | −72.092 | 1.00 | 60.51 | D |
| ATOM | 12113 | CG | LYS | D | 164 | 42.281 | 44.391 | −71.979 | 1.00 | 60.26 | D |
| ATOM | 12114 | CD | LYS | D | 164 | 42.057 | 43.762 | −73.347 | 1.00 | 60.26 | D |
| ATOM | 12115 | CE | LYS | D | 164 | 41.192 | 42.513 | −73.283 | 1.00 | 60.26 | D |
| ATOM | 12116 | NZ | LYS | D | 164 | 40.867 | 41.987 | −74.661 | 1.00 | 60.26 | D |
| ATOM | 12117 | C | LYS | D | 164 | 44.936 | 46.472 | −73.637 | 1.00 | 184.19 | D |
| ATOM | 12118 | O | LYS | D | 164 | 46.080 | 46.927 | −73.653 | 1.00 | 184.19 | D |
| ATOM | 12119 | N | ASP | D | 165 | 43.986 | 46.883 | −74.472 | 1.00 | 200.84 | D |
| ATOM | 12120 | CA | ASP | D | 165 | 44.258 | 47.940 | −75.435 | 1.00 | 200.84 | D |
| ATOM | 12121 | CB | ASP | D | 165 | 43.041 | 48.175 | −76.345 | 1.00 | 138.78 | D |
| ATOM | 12122 | CG | ASP | D | 165 | 41.740 | 48.305 | −75.569 | 1.00 | 138.78 | D |
| ATOM | 12123 | OD1 | ASP | D | 165 | 41.601 | 49.283 | −74.798 | 1.00 | 138.78 | D |
| ATOM | 12124 | OD2 | ASP | D | 165 | 40.860 | 47.424 | −75.732 | 1.00 | 138.78 | D |
| ATOM | 12125 | C | ASP | D | 165 | 44.583 | 49.190 | −74.628 | 1.00 | 200.84 | D |
| ATOM | 12126 | O | ASP | D | 165 | 45.362 | 49.135 | −73.676 | 1.00 | 200.84 | D |
| ATOM | 12127 | N | ASP | D | 166 | 43.988 | 50.318 | −74.983 | 1.00 | 102.07 | D |
| ATOM | 12128 | CA | ASP | D | 166 | 44.271 | 51.527 | −74.234 | 1.00 | 102.07 | D |
| ATOM | 12129 | CB | ASP | D | 166 | 44.336 | 52.722 | −75.184 | 1.00 | 200.84 | D |
| ATOM | 12130 | CG | ASP | D | 166 | 45.497 | 52.622 | −76.157 | 1.00 | 200.84 | D |
| ATOM | 12131 | OD1 | ASP | D | 166 | 45.532 | 51.651 | −76.943 | 1.00 | 200.84 | D |
| ATOM | 12132 | OD2 | ASP | D | 166 | 46.377 | 53.508 | −76.130 | 1.00 | 200.84 | D |
| ATOM | 12133 | C | ASP | D | 166 | 43.280 | 51.788 | −73.097 | 1.00 | 102.07 | D |
| ATOM | 12134 | O | ASP | D | 166 | 42.066 | 51.910 | −73.317 | 1.00 | 102.07 | D |
| ATOM | 12135 | N | GLY | D | 167 | 43.812 | 51.842 | −71.874 | 1.00 | 71.33 | D |
| ATOM | 12136 | CA | GLY | D | 167 | 42.978 | 52.120 | −70.726 | 1.00 | 71.33 | D |
| ATOM | 12137 | C | GLY | D | 167 | 43.217 | 51.377 | −69.423 | 1.00 | 71.33 | D |
| ATOM | 12138 | O | GLY | D | 167 | 44.273 | 51.443 | −68.782 | 1.00 | 71.33 | D |
| ATOM | 12139 | N | LEU | D | 168 | 42.170 | 50.667 | −69.035 | 1.00 | 45.34 | D |
| ATOM | 12140 | CA | LEU | D | 168 | 42.126 | 49.902 | −67.808 | 1.00 | 45.34 | D |
| ATOM | 12141 | CB | LEU | D | 168 | 40.664 | 49.556 | −67.530 | 1.00 | 63.06 | D |
| ATOM | 12142 | CG | LEU | D | 168 | 39.667 | 50.584 | −68.093 | 1.00 | 63.06 | D |
| ATOM | 12143 | CD1 | LEU | D | 168 | 38.250 | 50.098 | −67.904 | 1.00 | 63.06 | D |
| ATOM | 12144 | CD2 | LEU | D | 168 | 39.863 | 51.912 | −67.403 | 1.00 | 63.06 | D |
| ATOM | 12145 | C | LEU | D | 168 | 42.964 | 48.629 | −67.813 | 1.00 | 45.34 | D |
| ATOM | 12146 | O | LEU | D | 168 | 43.505 | 48.208 | −68.833 | 1.00 | 45.34 | D |
| ATOM | 12147 | N | TYR | D | 169 | 43.062 | 48.025 | −66.638 | 1.00 | 43.74 | D |
| ATOM | 12148 | CA | TYR | D | 169 | 43.800 | 46.787 | −66.471 | 1.00 | 43.74 | D |
| ATOM | 12149 | CB | TYR | D | 169 | 44.744 | 46.878 | −65.273 | 1.00 | 58.05 | D |
| ATOM | 12150 | CG | TYR | D | 169 | 45.911 | 47.813 | −65.455 | 1.00 | 58.05 | D |
| ATOM | 12151 | CD1 | TYR | D | 169 | 45.747 | 49.191 | −65.358 | 1.00 | 58.05 | D |
| ATOM | 12152 | CE1 | TYR | D | 169 | 46.821 | 50.051 | −65.528 | 1.00 | 58.05 | D |
| ATOM | 12153 | CD2 | TYR | D | 169 | 47.187 | 47.313 | −65.729 | 1.00 | 58.05 | D |
| ATOM | 12154 | CE2 | TYR | D | 169 | 48.270 | 48.159 | −65.901 | 1.00 | 58.05 | D |
| ATOM | 12155 | CZ | TYR | D | 169 | 48.084 | 49.530 | −65.800 | 1.00 | 58.05 | D |
| ATOM | 12156 | OH | TYR | D | 169 | 49.173 | 50.360 | −65.970 | 1.00 | 58.05 | D |
| ATOM | 12157 | C | TYR | D | 169 | 42.782 | 45.677 | −66.225 | 1.00 | 43.74 | D |
| ATOM | 12158 | O | TYR | D | 169 | 41.989 | 45.757 | −65.288 | 1.00 | 43.74 | D |
| ATOM | 12159 | N | ASP | D | 170 | 42.783 | 44.656 | −67.073 | 1.00 | 45.52 | D |
| ATOM | 12160 | CA | ASP | D | 170 | 41.852 | 43.564 | −66.880 | 1.00 | 45.52 | D |
| ATOM | 12161 | CB | ASP | D | 170 | 41.498 | 42.905 | −68.211 | 1.00 | 72.94 | D |
| ATOM | 12162 | CG | ASP | D | 170 | 40.239 | 43.477 | −68.824 | 1.00 | 72.94 | D |
| ATOM | 12163 | OD1 | ASP | D | 170 | 39.308 | 43.795 | −68.052 | 1.00 | 72.94 | D |
| ATOM | 12164 | OD2 | ASP | D | 170 | 40.182 | 43.588 | −70.071 | 1.00 | 72.94 | D |
| ATOM | 12165 | C | ASP | D | 170 | 42.467 | 42.531 | −65.962 | 1.00 | 45.52 | D |
| ATOM | 12166 | O | ASP | D | 170 | 43.688 | 42.423 | −65.866 | 1.00 | 45.52 | D |
| ATOM | 12167 | N | GLY | D | 171 | 41.623 | 41.776 | −65.273 | 1.00 | 47.44 | D |
| ATOM | 12168 | CA | GLY | D | 171 | 42.140 | 40.748 | −64.394 | 1.00 | 47.44 | D |
| ATOM | 12169 | C | GLY | D | 171 | 41.335 | 39.468 | −64.499 | 1.00 | 47.44 | D |
| ATOM | 12170 | O | GLY | D | 171 | 40.101 | 39.511 | −64.525 | 1.00 | 47.44 | D |
| ATOM | 12171 | N | VAL | D | 172 | 42.026 | 38.332 | −64.596 | 1.00 | 37.44 | D |
| ATOM | 12172 | CA | VAL | D | 172 | 41.368 | 37.030 | −64.650 | 1.00 | 37.44 | D |
| ATOM | 12173 | CB | VAL | D | 172 | 41.360 | 36.387 | −66.040 | 1.00 | 64.69 | D |
| ATOM | 12174 | CG1 | VAL | D | 172 | 40.191 | 36.883 | −66.815 | 1.00 | 56.70 | D |
| ATOM | 12175 | CG2 | VAL | D | 172 | 42.656 | 36.667 | −66.758 | 1.00 | 56.70 | D |
| ATOM | 12176 | C | VAL | D | 172 | 42.110 | 36.070 | −63.769 | 1.00 | 37.44 | D |
| ATOM | 12177 | O | VAL | D | 172 | 43.299 | 36.241 | −63.502 | 1.00 | 37.44 | D |
| ATOM | 12178 | N | PHE | D | 173 | 41.391 | 35.041 | −63.343 | 1.00 | 38.86 | D |
| ATOM | 12179 | CA | PHE | D | 173 | 41.936 | 34.000 | −62.516 | 1.00 | 38.86 | D |
| ATOM | 12180 | CB | PHE | D | 173 | 41.534 | 34.207 | −61.069 | 1.00 | 62.44 | D |
| ATOM | 12181 | CG | PHE | D | 173 | 41.807 | 33.020 | −60.225 | 1.00 | 44.26 | D |
| ATOM | 12182 | CD1 | PHE | D | 173 | 43.108 | 32.713 | −59.841 | 1.00 | 44.26 | D |
| ATOM | 12183 | CD2 | PHE | D | 173 | 40.782 | 32.136 | −59.903 | 1.00 | 44.26 | D |
| ATOM | 12184 | CE1 | PHE | D | 173 | 43.387 | 31.543 | −59.159 | 1.00 | 44.26 | D |
| ATOM | 12185 | CE2 | PHE | D | 173 | 41.049 | 30.955 | −59.217 | 1.00 | 44.26 | D |
| ATOM | 12186 | CZ | PHE | D | 173 | 42.353 | 30.655 | −58.845 | 1.00 | 44.26 | D |
| ATOM | 12187 | C | PHE | D | 173 | 41.377 | 32.679 | −62.985 | 1.00 | 38.86 | D |
| ATOM | 12188 | O | PHE | D | 173 | 40.181 | 32.575 | −63.246 | 1.00 | 38.86 | D |
| ATOM | 12189 | N | TYR | D | 174 | 42.235 | 31.671 | −63.098 | 1.00 | 38.88 | D |
| ATOM | 12190 | CA | TYR | D | 174 | 41.789 | 30.341 | −63.495 | 1.00 | 38.88 | D |
| ATOM | 12191 | CB | TYR | D | 174 | 42.307 | 29.958 | −64.883 | 1.00 | 58.27 | D |
| ATOM | 12192 | CG | TYR | D | 174 | 41.748 | 30.775 | −66.014 | 1.00 | 44.31 | D |
| ATOM | 12193 | CD1 | TYR | D | 174 | 42.526 | 31.749 | −66.637 | 1.00 | 44.31 | D |
| ATOM | 12194 | CE1 | TYR | D | 174 | 42.020 | 32.523 | −67.687 | 1.00 | 44.31 | D |
| ATOM | 12195 | CD2 | TYR | D | 174 | 40.435 | 30.585 | −66.466 | 1.00 | 44.31 | D |
| ATOM | 12196 | CE2 | TYR | D | 174 | 39.914 | 31.356 | −67.521 | 1.00 | 44.31 | D |
| ATOM | 12197 | CZ | TYR | D | 174 | 40.719 | 32.323 | −68.124 | 1.00 | 44.31 | D |
| ATOM | 12198 | OH | TYR | D | 174 | 40.247 | 33.090 | −69.168 | 1.00 | 44.31 | D |
| ATOM | 12199 | C | TYR | D | 174 | 42.264 | 29.294 | −62.496 | 1.00 | 38.88 | D |
| ATOM | 12200 | O | TYR | D | 174 | 43.396 | 29.342 | −62.039 | 1.00 | 38.88 | D |
| ATOM | 12201 | N | ASP | D | 175 | 41.377 | 28.361 | −62.163 | 1.00 | 39.24 | D |
| ATOM | 12202 | CA | ASP | D | 175 | 41.664 | 27.258 | −61.259 | 1.00 | 39.24 | D |
| ATOM | 12203 | CB | ASP | D | 175 | 40.637 | 27.222 | −60.122 | 1.00 | 67.75 | D |
| ATOM | 12204 | CG | ASP | D | 175 | 40.954 | 26.166 | −59.070 | 1.00 | 55.96 | D |
| ATOM | 12205 | OD1 | ASP | D | 175 | 41.502 | 25.101 | −59.426 | 1.00 | 55.96 | D |
| ATOM | 12206 | OD2 | ASP | D | 175 | 40.638 | 26.393 | −57.881 | 1.00 | 55.96 | D |
| ATOM | 12207 | C | ASP | D | 175 | 41.525 | 25.995 | −62.124 | 1.00 | 39.24 | D |
| ATOM | 12208 | O | ASP | D | 175 | 40.434 | 25.669 | −62.597 | 1.00 | 39.24 | D |
| ATOM | 12209 | N | HIS | D | 176 | 42.626 | 25.292 | −62.350 | 1.00 | 47.20 | D |
| ATOM | 12210 | CA | HIS | D | 176 | 42.582 | 24.083 | −63.151 | 1.00 | 47.20 | D |
| ATOM | 12211 | CB | HIS | D | 176 | 43.473 | 24.224 | −64.381 | 1.00 | 75.10 | D |
| ATOM | 12212 | CG | HIS | D | 176 | 42.900 | 25.082 | −65.462 | 1.00 | 44.23 | D |
| ATOM | 12213 | CD2 | HIS | D | 176 | 43.241 | 26.322 | −65.890 | 1.00 | 44.23 | D |
| ATOM | 12214 | ND1 | HIS | D | 176 | 41.859 | 24.673 | −66.267 | 1.00 | 44.23 | D |
| ATOM | 12215 | CE1 | HIS | D | 176 | 41.584 | 25.621 | −67.145 | 1.00 | 44.23 | D |
| ATOM | 12216 | NE2 | HIS | D | 176 | 42.407 | 26.632 | −66.938 | 1.00 | 44.23 | D |
| ATOM | 12217 | C | HIS | D | 176 | 43.032 | 22.838 | −62.385 | 1.00 | 47.20 | D |
| ATOM | 12218 | O | HIS | D | 176 | 44.113 | 22.828 | −61.788 | 1.00 | 47.20 | D |
| ATOM | 12219 | N | TYR | D | 177 | 42.191 | 21.799 | −62.392 | 1.00 | 28.41 | D |
| ATOM | 12220 | CA | TYR | D | 177 | 42.531 | 20.522 | −61.783 | 1.00 | 28.41 | D |
| ATOM | 12221 | CB | TYR | D | 177 | 41.365 | 19.937 | −61.011 | 1.00 | 47.01 | D |
| ATOM | 12222 | CG | TYR | D | 177 | 41.696 | 18.576 | −60.474 | 1.00 | 40.02 | D |
| ATOM | 12223 | CD1 | TYR | D | 177 | 42.641 | 18.422 | −59.463 | 1.00 | 40.02 | D |
| ATOM | 12224 | CE1 | TYR | D | 177 | 42.992 | 17.167 | −58.992 | 1.00 | 40.02 | D |
| ATOM | 12225 | CD2 | TYR | D | 177 | 41.102 | 17.435 | −61.002 | 1.00 | 40.02 | D |
| ATOM | 12226 | CE2 | TYR | D | 177 | 41.440 | 16.176 | −60.539 | 1.00 | 40.02 | D |
| ATOM | 12227 | CZ | TYR | D | 177 | 42.390 | 16.047 | −59.532 | 1.00 | 40.02 | D |
| ATOM | 12228 | OH | TYR | D | 177 | 42.729 | 14.793 | −59.075 | 1.00 | 40.02 | D |
| ATOM | 12229 | C | TYR | D | 177 | 42.802 | 19.670 | −63.022 | 1.00 | 28.41 | D |
| ATOM | 12230 | O | TYR | D | 177 | 41.898 | 19.430 | −63.808 | 1.00 | 28.41 | D |
| ATOM | 12231 | N | ASN | D | 178 | 44.036 | 19.212 | −63.200 | 1.00 | 46.18 | D |
| ATOM | 12232 | CA | ASN | D | 178 | 44.381 | 18.459 | −64.397 | 1.00 | 46.18 | D |
| ATOM | 12233 | CB | ASN | D | 178 | 43.535 | 17.201 | −64.553 | 1.00 | 47.19 | D |
| ATOM | 12234 | CG | ASN | D | 178 | 43.958 | 16.090 | −63.616 | 1.00 | 39.66 | D |
| ATOM | 12235 | OD1 | ASN | D | 178 | 45.007 | 16.167 | −62.961 | 1.00 | 39.66 | D |
| ATOM | 12236 | ND2 | ASN | D | 178 | 43.147 | 15.035 | −63.556 | 1.00 | 39.66 | D |
| ATOM | 12237 | C | ASN | D | 178 | 44.032 | 19.433 | −65.500 | 1.00 | 46.18 | D |
| ATOM | 12238 | O | ASN | D | 178 | 44.343 | 20.618 | −65.395 | 1.00 | 46.18 | D |
| ATOM | 12239 | N | ASP | D | 179 | 43.379 | 18.958 | −66.552 | 1.00 | 35.34 | D |
| ATOM | 12240 | CA | ASP | D | 179 | 43.002 | 19.864 | −67.629 | 1.00 | 35.34 | D |
| ATOM | 12241 | CB | ASP | D | 179 | 43.185 | 19.192 | −69.004 | 1.00 | 35.01 | D |
| ATOM | 12242 | CG | ASP | D | 179 | 42.475 | 17.855 | −69.123 | 1.00 | 38.96 | D |
| ATOM | 12243 | OD1 | ASP | D | 179 | 41.787 | 17.431 | −68.170 | 1.00 | 38.96 | D |
| ATOM | 12244 | OD2 | ASP | D | 179 | 42.611 | 17.216 | −70.189 | 1.00 | 38.96 | D |
| ATOM | 12245 | C | ASP | D | 179 | 41.571 | 20.376 | −67.474 | 1.00 | 35.34 | D |
| ATOM | 12246 | O | ASP | D | 179 | 41.023 | 20.957 | −68.389 | 1.00 | 35.34 | D |
| ATOM | 12247 | N | GLN | D | 180 | 40.984 | 20.170 | −66.298 | 1.00 | 41.96 | D |
| ATOM | 12248 | CA | GLN | D | 180 | 39.614 | 20.587 | −66.017 | 1.00 | 41.96 | D |
| ATOM | 12249 | CB | GLN | D | 180 | 38.947 | 19.570 | −65.094 | 1.00 | 58.83 | D |
| ATOM | 12250 | CG | GLN | D | 180 | 38.991 | 18.155 | −65.579 | 1.00 | 50.46 | D |
| ATOM | 12251 | CD | GLN | D | 180 | 38.333 | 18.004 | −66.921 | 1.00 | 50.46 | D |
| ATOM | 12252 | OE1 | GLN | D | 180 | 39.000 | 17.914 | −67.946 | 1.00 | 50.46 | D |
| ATOM | 12253 | NE2 | GLN | D | 180 | 37.011 | 17.987 | −66.926 | 1.00 | 50.46 | D |
| ATOM | 12254 | C | GLN | D | 180 | 39.471 | 21.972 | −65.368 | 1.00 | 41.96 | D |
| ATOM | 12255 | O | GLN | D | 180 | 39.978 | 22.209 | −64.270 | 1.00 | 41.96 | D |
| ATOM | 12256 | N | LEU | D | 181 | 38.759 | 22.876 | −66.033 | 1.00 | 37.16 | D |
| ATOM | 12257 | CA | LEU | D | 181 | 38.531 | 24.200 | −65.476 | 1.00 | 37.16 | D |
| ATOM | 12258 | CB | LEU | D | 181 | 37.873 | 25.113 | −66.504 | 1.00 | 52.14 | D |
| ATOM | 12259 | CG | LEU | D | 181 | 37.491 | 26.485 | −65.942 | 1.00 | 39.57 | D |
| ATOM | 12260 | CD1 | LEU | D | 181 | 38.735 | 27.233 | −65.458 | 1.00 | 39.57 | D |
| ATOM | 12261 | CD2 | LEU | D | 181 | 36.756 | 27.283 | −67.005 | 1.00 | 39.57 | D |
| ATOM | 12262 | C | LEU | D | 181 | 37.621 | 24.047 | −64.262 | 1.00 | 37.16 | D |
| ATOM | 12263 | O | LEU | D | 181 | 36.590 | 23.401 | −64.341 | 1.00 | 37.16 | D |
| ATOM | 12264 | N | VAL | D | 182 | 38.010 | 24.638 | −63.140 | 1.00 | 35.48 | D |
| ATOM | 12265 | CA | VAL | D | 182 | 37.225 | 24.531 | −61.919 | 1.00 | 35.48 | D |
| ATOM | 12266 | CB | VAL | D | 182 | 38.103 | 24.099 | −60.726 | 1.00 | 50.96 | D |
| ATOM | 12267 | CG1 | VAL | D | 182 | 37.268 | 24.068 | −59.461 | 1.00 | 29.44 | D |
| ATOM | 12268 | CG2 | VAL | D | 182 | 38.719 | 22.719 | −60.995 | 1.00 | 29.44 | D |
| ATOM | 12269 | C | VAL | D | 182 | 36.577 | 25.850 | −61.574 | 1.00 | 35.48 | D |
| ATOM | 12270 | O | VAL | D | 182 | 35.422 | 25.890 | −61.154 | 1.00 | 35.48 | D |
| ATOM | 12271 | N | THR | D | 183 | 37.327 | 26.933 | −61.756 | 1.00 | 40.82 | D |
| ATOM | 12272 | CA | THR | D | 183 | 36.839 | 28.268 | −61.438 | 1.00 | 40.82 | D |
| ATOM | 12273 | CB | THR | D | 183 | 37.199 | 28.664 | −59.994 | 1.00 | 54.92 | D |
| ATOM | 12274 | OG1 | THR | D | 183 | 36.506 | 27.820 | −59.077 | 1.00 | 41.51 | D |
| ATOM | 12275 | CG2 | THR | D | 183 | 36.825 | 30.101 | −59.720 | 1.00 | 41.51 | D |
| ATOM | 12276 | C | THR | D | 183 | 37.435 | 29.333 | −62.329 | 1.00 | 40.82 | D |
| ATOM | 12277 | O | THR | D | 183 | 38.618 | 29.284 | −62.655 | 1.00 | 40.82 | D |
| ATOM | 12278 | N | LYS | D | 184 | 36.604 | 30.285 | −62.737 | 1.00 | 40.43 | D |
| ATOM | 12279 | CA | LYS | D | 184 | 37.088 | 31.410 | −63.513 | 1.00 | 40.43 | D |
| ATOM | 12280 | CB | LYS | D | 184 | 36.581 | 31.400 | −64.934 | 1.00 | 46.60 | D |
| ATOM | 12281 | CG | LYS | D | 184 | 37.062 | 32.626 | −65.684 | 1.00 | 38.44 | D |
| ATOM | 12282 | CD | LYS | D | 184 | 36.494 | 32.716 | −67.082 | 1.00 | 38.44 | D |
| ATOM | 12283 | CE | LYS | D | 184 | 36.938 | 34.002 | −67.750 | 1.00 | 38.44 | D |
| ATOM | 12284 | NZ | LYS | D | 184 | 36.327 | 34.169 | −69.094 | 1.00 | 38.44 | D |
| ATOM | 12285 | C | LYS | D | 184 | 36.574 | 32.663 | −62.821 | 1.00 | 40.43 | D |
| ATOM | 12286 | O | LYS | D | 184 | 35.403 | 32.750 | −62.450 | 1.00 | 40.43 | D |
| ATOM | 12287 | N | ILE | D | 185 | 37.459 | 33.627 | −62.624 | 1.00 | 53.56 | D |
| ATOM | 12288 | CA | ILE | D | 185 | 37.085 | 34.868 | −61.975 | 1.00 | 53.56 | D |
| ATOM | 12289 | CB | ILE | D | 185 | 37.628 | 34.926 | −60.531 | 1.00 | 53.69 | D |
| ATOM | 12290 | CG2 | ILE | D | 185 | 37.491 | 36.341 | −59.965 | 1.00 | 22.61 | D |
| ATOM | 12291 | CG1 | ILE | D | 185 | 36.877 | 33.916 | −59.654 | 1.00 | 22.61 | D |
| ATOM | 12292 | CD1 | ILE | D | 185 | 37.437 | 33.759 | −58.245 | 1.00 | 22.61 | D |
| ATOM | 12293 | C | ILE | D | 185 | 37.663 | 36.019 | −62.775 | 1.00 | 53.56 | D |
| ATOM | 12294 | O | ILE | D | 185 | 38.790 | 35.941 | −63.268 | 1.00 | 53.56 | D |
| ATOM | 12295 | N | SER | D | 186 | 36.884 | 37.081 | −62.934 | 1.00 | 34.32 | D |
| ATOM | 12296 | CA | SER | D | 186 | 37.363 | 38.240 | −63.666 | 1.00 | 34.32 | D |
| ATOM | 12297 | CB | SER | D | 186 | 36.830 | 38.229 | −65.097 | 1.00 | 55.84 | D |
| ATOM | 12298 | OG | SER | D | 186 | 35.463 | 37.913 | −65.102 | 1.00 | 44.95 | D |
| ATOM | 12299 | C | SER | D | 186 | 36.989 | 39.527 | −62.937 | 1.00 | 34.32 | D |
| ATOM | 12300 | O | SER | D | 186 | 36.008 | 39.594 | −62.193 | 1.00 | 34.32 | D |
| ATOM | 12301 | N | TYR | D | 187 | 37.817 | 40.541 | −63.125 | 1.00 | 48.86 | D |
| ATOM | 12302 | CA | TYR | D | 187 | 37.610 | 41.823 | −62.486 | 1.00 | 48.86 | D |
| ATOM | 12303 | CB | TYR | D | 187 | 38.894 | 42.625 | −62.533 | 1.00 | 89.31 | D |
| ATOM | 12304 | CG | TYR | D | 187 | 38.787 | 43.906 | −61.774 | 1.00 | 43.52 | D |
| ATOM | 12305 | CD1 | TYR | D | 187 | 38.743 | 43.903 | −60.390 | 1.00 | 43.52 | D |
| ATOM | 12306 | CE1 | TYR | D | 187 | 38.664 | 45.088 | −59.670 | 1.00 | 43.52 | D |
| ATOM | 12307 | CD2 | TYR | D | 187 | 38.740 | 45.131 | −62.435 | 1.00 | 43.52 | D |
| ATOM | 12308 | CE2 | TYR | D | 187 | 38.660 | 46.322 | −61.725 | 1.00 | 43.52 | D |
| ATOM | 12309 | CZ | TYR | D | 187 | 38.625 | 46.293 | −60.340 | 1.00 | 43.52 | D |
| ATOM | 12310 | OH | TYR | D | 187 | 38.572 | 47.467 | −59.622 | 1.00 | 43.52 | D |
| ATOM | 12311 | C | TYR | D | 187 | 36.504 | 42.621 | −63.160 | 1.00 | 48.86 | D |
| ATOM | 12312 | O | TYR | D | 187 | 36.583 | 42.949 | −64.347 | 1.00 | 48.86 | D |
| ATOM | 12313 | N | ASN | D | 188 | 35.480 | 42.939 | −62.386 | 1.00 | 47.79 | D |
| ATOM | 12314 | CA | ASN | D | 188 | 34.343 | 43.709 | −62.862 | 1.00 | 47.79 | D |
| ATOM | 12315 | CB | ASN | D | 188 | 33.136 | 43.361 | −62.008 | 1.00 | 184.83 | D |
| ATOM | 12316 | CG | ASN | D | 188 | 31.912 | 44.090 | −62.427 | 1.00 | 45.86 | D |
| ATOM | 12317 | OD1 | ASN | D | 188 | 31.928 | 45.298 | −62.590 | 1.00 | 45.86 | D |
| ATOM | 12318 | ND2 | ASN | D | 188 | 30.833 | 43.367 | −62.606 | 1.00 | 45.86 | D |
| ATOM | 12319 | C | ASN | D | 188 | 34.659 | 45.216 | −62.745 | 1.00 | 47.79 | D |
| ATOM | 12320 | O | ASN | D | 188 | 34.754 | 45.751 | −61.636 | 1.00 | 47.79 | D |
| ATOM | 12321 | N | HIS | D | 189 | 34.816 | 45.905 | −63.876 | 1.00 | 42.42 | D |
| ATOM | 12322 | CA | HIS | D | 189 | 35.148 | 47.326 | −63.848 | 1.00 | 42.42 | D |
| ATOM | 12323 | CB | HIS | D | 189 | 35.568 | 47.765 | −65.233 | 1.00 | 97.40 | D |
| ATOM | 12324 | CG | HIS | D | 189 | 36.906 | 47.243 | −65.614 | 1.00 | 57.94 | D |
| ATOM | 12325 | CD2 | HIS | D | 189 | 37.266 | 46.181 | −66.371 | 1.00 | 57.94 | D |
| ATOM | 12326 | ND1 | HIS | D | 189 | 38.075 | 47.787 | −65.132 | 1.00 | 57.94 | D |
| ATOM | 12327 | CE1 | HIS | D | 189 | 39.100 | 47.083 | −65.579 | 1.00 | 57.94 | D |
| ATOM | 12328 | NE2 | HIS | D | 189 | 38.636 | 46.102 | −66.333 | 1.00 | 57.94 | D |
| ATOM | 12329 | C | HIS | D | 189 | 34.060 | 48.234 | −63.303 | 1.00 | 42.42 | D |
| ATOM | 12330 | O | HIS | D | 189 | 34.347 | 49.293 | −62.759 | 1.00 | 42.42 | D |
| ATOM | 12331 | N | GLU | D | 190 | 32.811 | 47.813 | −63.444 | 1.00 | 66.35 | D |
| ATOM | 12332 | CA | GLU | D | 190 | 31.681 | 48.591 | −62.962 | 1.00 | 66.35 | D |
| ATOM | 12333 | CB | GLU | D | 190 | 30.389 | 47.992 | −63.515 | 1.00 | 122.49 | D |
| ATOM | 12334 | CG | GLU | D | 190 | 29.219 | 48.938 | −63.578 | 1.00 | 96.91 | D |
| ATOM | 12335 | CD | GLU | D | 190 | 28.018 | 48.300 | −64.246 | 1.00 | 96.91 | D |
| ATOM | 12336 | OE1 | GLU | D | 190 | 27.470 | 47.333 | −63.676 | 1.00 | 96.91 | D |
| ATOM | 12337 | OE2 | GLU | D | 190 | 27.627 | 48.754 | −65.347 | 1.00 | 96.91 | D |
| ATOM | 12338 | C | GLU | D | 190 | 31.694 | 48.521 | −61.432 | 1.00 | 66.35 | D |
| ATOM | 12339 | O | GLU | D | 190 | 31.738 | 49.530 | −60.746 | 1.00 | 66.35 | D |
| ATOM | 12340 | N | THR | D | 191 | 31.688 | 47.308 | −60.907 | 1.00 | 51.53 | D |
| ATOM | 12341 | CA | THR | D | 191 | 31.690 | 47.056 | −59.472 | 1.00 | 51.53 | D |
| ATOM | 12342 | CB | THR | D | 191 | 31.247 | 45.631 | −59.193 | 1.00 | 64.06 | D |
| ATOM | 12343 | OG1 | THR | D | 191 | 29.907 | 45.459 | −59.649 | 1.00 | 64.06 | D |
| ATOM | 12344 | CG2 | THR | D | 191 | 31.283 | 45.348 | −57.737 | 1.00 | 64.06 | D |
| ATOM | 12345 | C | THR | D | 191 | 33.030 | 47.264 | −58.765 | 1.00 | 51.53 | D |
| ATOM | 12346 | O | THR | D | 191 | 33.069 | 47.570 | −57.578 | 1.00 | 51.53 | D |
| ATOM | 12347 | N | ARG | D | 192 | 34.131 | 47.071 | −59.480 | 1.00 | 72.64 | D |
| ATOM | 12348 | CA | ARG | D | 192 | 35.469 | 47.248 | −58.909 | 1.00 | 72.64 | D |
| ATOM | 12349 | CB | ARG | D | 192 | 35.559 | 48.578 | −58.152 | 1.00 | 131.19 | D |
| ATOM | 12350 | CG | ARG | D | 192 | 35.435 | 49.792 | −59.047 | 1.00 | 131.19 | D |
| ATOM | 12351 | CD | ARG | D | 192 | 36.501 | 49.764 | −60.142 | 1.00 | 131.19 | D |
| ATOM | 12352 | NE | ARG | D | 192 | 36.301 | 50.808 | −61.145 | 1.00 | 131.19 | D |
| ATOM | 12353 | CZ | ARG | D | 192 | 36.372 | 52.113 | −60.895 | 1.00 | 131.19 | D |
| ATOM | 12354 | NH1 | ARG | D | 192 | 36.641 | 52.547 | −59.670 | 1.00 | 131.19 | D |
| ATOM | 12355 | NH2 | ARG | D | 192 | 36.166 | 52.987 | −61.871 | 1.00 | 131.19 | D |
| ATOM | 12356 | C | ARG | D | 192 | 35.943 | 46.103 | −58.008 | 1.00 | 72.64 | D |
| ATOM | 12357 | O | ARG | D | 192 | 36.728 | 46.310 | −57.092 | 1.00 | 72.64 | D |
| ATOM | 12358 | N | HIS | D | 193 | 35.457 | 44.896 | −58.278 | 1.00 | 57.71 | D |
| ATOM | 12359 | CA | HIS | D | 193 | 35.861 | 43.711 | −57.535 | 1.00 | 57.71 | D |
| ATOM | 12360 | CB | HIS | D | 193 | 35.064 | 43.601 | −56.235 | 1.00 | 119.34 | D |
| ATOM | 12361 | CG | HIS | D | 193 | 33.635 | 43.201 | −56.420 | 1.00 | 119.34 | D |
| ATOM | 12362 | CD2 | HIS | D | 193 | 32.984 | 42.620 | −57.460 | 1.00 | 119.34 | D |
| ATOM | 12363 | ND1 | HIS | D | 193 | 32.703 | 43.338 | −55.416 | 1.00 | 119.34 | D |
| ATOM | 12364 | CE1 | HIS | D | 193 | 31.543 | 42.859 | −55.829 | 1.00 | 119.34 | D |
| ATOM | 12365 | NE2 | HIS | D | 193 | 31.684 | 42.417 | −57.065 | 1.00 | 119.34 | D |
| ATOM | 12366 | C | HIS | D | 193 | 35.655 | 42.501 | −58.451 | 1.00 | 57.71 | D |
| ATOM | 12367 | O | HIS | D | 193 | 35.044 | 42.635 | −59.512 | 1.00 | 57.71 | D |
| ATOM | 12368 | N | GLY | D | 194 | 36.156 | 41.331 | −58.066 | 1.00 | 37.14 | D |
| ATOM | 12369 | CA | GLY | D | 194 | 36.022 | 40.155 | −58.923 | 1.00 | 37.14 | D |
| ATOM | 12370 | C | GLY | D | 194 | 34.670 | 39.462 | −58.907 | 1.00 | 37.14 | D |
| ATOM | 12371 | O | GLY | D | 194 | 33.875 | 39.663 | −58.000 | 1.00 | 37.14 | D |
| ATOM | 12372 | N | ASN | D | 195 | 34.389 | 38.659 | −59.924 | 1.00 | 40.86 | D |
| ATOM | 12373 | CA | ASN | D | 195 | 33.131 | 37.931 | −59.982 | 1.00 | 40.86 | D |
| ATOM | 12374 | CB | ASN | D | 195 | 32.149 | 38.557 | −60.982 | 1.00 | 70.65 | D |
| ATOM | 12375 | CG | ASN | D | 195 | 31.599 | 39.881 | −60.523 | 1.00 | 70.65 | D |
| ATOM | 12376 | OD1 | ASN | D | 195 | 31.225 | 40.045 | −59.369 | 1.00 | 70.65 | D |
| ATOM | 12377 | ND2 | ASN | D | 195 | 31.529 | 40.835 | −61.437 | 1.00 | 70.65 | D |
| ATOM | 12378 | C | ASN | D | 195 | 33.450 | 36.539 | −60.473 | 1.00 | 40.86 | D |
| ATOM | 12379 | O | ASN | D | 195 | 34.307 | 36.367 | −61.350 | 1.00 | 40.86 | D |
| ATOM | 12380 | N | VAL | D | 196 | 32.774 | 35.537 | −59.922 | 1.00 | 41.78 | D |
| ATOM | 12381 | CA | VAL | D | 196 | 32.998 | 34.181 | −60.387 | 1.00 | 41.78 | D |
| ATOM | 12382 | CB | VAL | D | 196 | 32.537 | 33.159 | −59.365 | 1.00 | 39.68 | D |
| ATOM | 12383 | CG1 | VAL | D | 196 | 32.717 | 31.756 | −59.930 | 1.00 | 39.68 | D |
| ATOM | 12384 | CG2 | VAL | D | 196 | 33.323 | 33.328 | −58.082 | 1.00 | 39.68 | D |
| ATOM | 12385 | C | VAL | D | 196 | 32.169 | 34.017 | −61.650 | 1.00 | 41.78 | D |
| ATOM | 12386 | O | VAL | D | 196 | 30.950 | 34.170 | −61.618 | 1.00 | 41.78 | D |
| ATOM | 12387 | N | ASN | D | 197 | 32.828 | 33.728 | −62.764 | 1.00 | 46.78 | D |
| ATOM | 12388 | CA | ASN | D | 197 | 32.123 | 33.557 | −64.024 | 1.00 | 46.78 | D |
| ATOM | 12389 | CB | ASN | D | 197 | 32.967 | 34.068 | −65.189 | 1.00 | 48.10 | D |
| ATOM | 12390 | CG | ASN | D | 197 | 33.585 | 35.429 | −64.910 | 1.00 | 48.10 | D |
| ATOM | 12391 | OD1 | ASN | D | 197 | 32.908 | 36.357 | −64.462 | 1.00 | 48.10 | D |
| ATOM | 12392 | ND2 | ASN | D | 197 | 34.881 | 35.553 | −65.181 | 1.00 | 48.10 | D |
| ATOM | 12393 | C | ASN | D | 197 | 31.812 | 32.102 | −64.260 | 1.00 | 46.78 | D |
| ATOM | 12394 | O | ASN | D | 197 | 30.800 | 31.774 | −64.860 | 1.00 | 46.78 | D |
| ATOM | 12395 | N | PHE | D | 198 | 32.689 | 31.227 | −63.785 | 1.00 | 41.56 | D |
| ATOM | 12396 | CA | PHE | D | 198 | 32.506 | 29.804 | −63.976 | 1.00 | 41.56 | D |
| ATOM | 12397 | CB | PHE | D | 198 | 33.380 | 29.327 | −65.126 | 1.00 | 35.64 | D |
| ATOM | 12398 | CG | PHE | D | 198 | 33.228 | 27.882 | −65.429 | 1.00 | 35.64 | D |
| ATOM | 12399 | CD1 | PHE | D | 198 | 32.295 | 27.450 | −66.353 | 1.00 | 35.64 | D |
| ATOM | 12400 | CD2 | PHE | D | 198 | 33.987 | 26.940 | −64.751 | 1.00 | 35.64 | D |
| ATOM | 12401 | CE1 | PHE | D | 198 | 32.115 | 26.094 | −66.594 | 1.00 | 35.64 | D |
| ATOM | 12402 | CE2 | PHE | D | 198 | 33.811 | 25.577 | −64.989 | 1.00 | 35.64 | D |
| ATOM | 12403 | CZ | PHE | D | 198 | 32.875 | 25.156 | −65.908 | 1.00 | 35.64 | D |
| ATOM | 12404 | C | PHE | D | 198 | 32.862 | 29.010 | −62.743 | 1.00 | 41.56 | D |
| ATOM | 12405 | O | PHE | D | 198 | 33.786 | 29.369 | −62.009 | 1.00 | 41.56 | D |
| ATOM | 12406 | N | ARG | D | 199 | 32.121 | 27.929 | −62.524 | 1.00 | 43.89 | D |
| ATOM | 12407 | CA | ARG | D | 199 | 32.376 | 27.038 | −61.411 | 1.00 | 43.89 | D |
| ATOM | 12408 | CB | ARG | D | 199 | 31.590 | 27.453 | −60.177 | 1.00 | 48.28 | D |
| ATOM | 12409 | CG | ARG | D | 199 | 31.829 | 26.507 | −59.033 | 1.00 | 48.28 | D |
| ATOM | 12410 | CD | ARG | D | 199 | 33.203 | 26.721 | −58.427 | 1.00 | 48.28 | D |
| ATOM | 12411 | NE | ARG | D | 199 | 33.077 | 27.527 | −57.223 | 1.00 | 48.28 | D |
| ATOM | 12412 | CZ | ARG | D | 199 | 33.942 | 28.456 | −56.856 | 1.00 | 48.28 | D |
| ATOM | 12413 | NH1 | ARG | D | 199 | 35.001 | 28.695 | −57.605 | 1.00 | 48.28 | D |
| ATOM | 12414 | NH2 | ARG | D | 199 | 33.742 | 29.162 | −55.747 | 1.00 | 48.28 | D |
| ATOM | 12415 | C | ARG | D | 199 | 31.964 | 25.637 | −61.836 | 1.00 | 43.89 | D |
| ATOM | 12416 | O | ARG | D | 199 | 30.810 | 25.402 | −62.163 | 1.00 | 43.89 | D |
| ATOM | 12417 | N | ALA | D | 200 | 32.909 | 24.709 | −61.851 | 1.00 | 42.10 | D |
| ATOM | 12418 | CA | ALA | D | 200 | 32.597 | 23.341 | −62.236 | 1.00 | 42.10 | D |
| ATOM | 12419 | CB | ALA | D | 200 | 33.857 | 22.471 | −62.167 | 1.00 | 53.77 | D |
| ATOM | 12420 | C | ALA | D | 200 | 31.506 | 22.775 | −61.319 | 1.00 | 42.10 | D |
| ATOM | 12421 | O | ALA | D | 200 | 31.538 | 22.951 | −60.098 | 1.00 | 42.10 | D |
| ATOM | 12422 | N | ASP | D | 201 | 30.539 | 22.099 | −61.928 | 1.00 | 47.43 | D |
| ATOM | 12423 | CA | ASP | D | 201 | 29.422 | 21.493 | −61.218 | 1.00 | 47.43 | D |
| ATOM | 12424 | CB | ASP | D | 201 | 28.494 | 20.822 | −62.229 | 1.00 | 101.04 | D |
| ATOM | 12425 | CG | ASP | D | 201 | 29.249 | 19.906 | −63.193 | 1.00 | 101.04 | D |
| ATOM | 12426 | OD1 | ASP | D | 201 | 30.037 | 20.431 | −64.016 | 1.00 | 101.04 | D |
| ATOM | 12427 | OD2 | ASP | D | 201 | 29.062 | 18.665 | −63.124 | 1.00 | 101.04 | D |
| ATOM | 12428 | C | ASP | D | 201 | 29.842 | 20.465 | −60.166 | 1.00 | 47.43 | D |
| ATOM | 12429 | O | ASP | D | 201 | 29.140 | 20.279 | −59.178 | 1.00 | 47.43 | D |
| ATOM | 12430 | N | TRP | D | 202 | 30.977 | 19.801 | −60.368 | 1.00 | 53.49 | D |
| ATOM | 12431 | CA | TRP | D | 202 | 31.426 | 18.780 | −59.428 | 1.00 | 53.49 | D |
| ATOM | 12432 | CB | TRP | D | 202 | 32.245 | 17.713 | −60.154 | 1.00 | 41.10 | D |
| ATOM | 12433 | CG | TRP | D | 202 | 33.402 | 18.243 | −60.956 | 1.00 | 41.10 | D |
| ATOM | 12434 | CD2 | TRP | D | 202 | 34.734 | 18.482 | −60.487 | 1.00 | 41.10 | D |
| ATOM | 12435 | CE2 | TRP | D | 202 | 35.489 | 18.926 | −61.591 | 1.00 | 41.10 | D |
| ATOM | 12436 | CE3 | TRP | D | 202 | 35.362 | 18.360 | −59.239 | 1.00 | 41.10 | D |
| ATOM | 12437 | CD1 | TRP | D | 202 | 33.406 | 18.553 | −62.284 | 1.00 | 41.10 | D |
| ATOM | 12438 | NE1 | TRP | D | 202 | 34.653 | 18.958 | −62.676 | 1.00 | 41.10 | D |
| ATOM | 12439 | CZ2 | TRP | D | 202 | 36.841 | 19.247 | −61.488 | 1.00 | 41.10 | D |
| ATOM | 12440 | CZ3 | TRP | D | 202 | 36.703 | 18.679 | −59.138 | 1.00 | 41.10 | D |
| ATOM | 12441 | CH2 | TRP | D | 202 | 37.429 | 19.117 | −60.258 | 1.00 | 41.10 | D |
| ATOM | 12442 | C | TRP | D | 202 | 32.233 | 19.267 | −58.240 | 1.00 | 53.49 | D |
| ATOM | 12443 | O | TRP | D | 202 | 32.522 | 18.492 | −57.328 | 1.00 | 53.49 | D |
| ATOM | 12444 | N | PHE | D | 203 | 32.593 | 20.542 | −58.243 | 1.00 | 41.86 | D |
| ATOM | 12445 | CA | PHE | D | 203 | 33.404 | 21.092 | −57.169 | 1.00 | 41.86 | D |
| ATOM | 12446 | CB | PHE | D | 203 | 34.487 | 21.996 | −57.776 | 1.00 | 40.90 | D |
| ATOM | 12447 | CG | PHE | D | 203 | 35.513 | 22.465 | −56.794 | 1.00 | 40.90 | D |
| ATOM | 12448 | CD1 | PHE | D | 203 | 36.429 | 21.570 | −56.236 | 1.00 | 40.90 | D |
| ATOM | 12449 | CD2 | PHE | D | 203 | 35.573 | 23.794 | −56.427 | 1.00 | 40.90 | D |
| ATOM | 12450 | CE1 | PHE | D | 203 | 37.402 | 22.003 | −55.321 | 1.00 | 40.90 | D |
| ATOM | 12451 | CE2 | PHE | D | 203 | 36.532 | 24.238 | −55.517 | 1.00 | 40.90 | D |
| ATOM | 12452 | CZ | PHE | D | 203 | 37.449 | 23.339 | −54.964 | 1.00 | 40.90 | D |
| ATOM | 12453 | C | PHE | D | 203 | 32.605 | 21.871 | −56.127 | 1.00 | 41.86 | D |
| ATOM | 12454 | O | PHE | D | 203 | 32.136 | 22.974 | −56.407 | 1.00 | 41.86 | D |
| ATOM | 12455 | N | ASN | D | 204 | 32.434 | 21.289 | −54.937 | 1.00 | 45.41 | D |
| ATOM | 12456 | CA | ASN | D | 204 | 31.731 | 21.962 | −53.842 | 1.00 | 45.41 | D |
| ATOM | 12457 | CB | ASN | D | 204 | 30.953 | 20.960 | −52.966 | 1.00 | 73.83 | D |
| ATOM | 12458 | CG | ASN | D | 204 | 30.436 | 21.591 | −51.675 | 1.00 | 73.83 | D |
| ATOM | 12459 | OD1 | ASN | D | 204 | 31.225 | 22.001 | −50.833 | 1.00 | 73.83 | D |
| ATOM | 12460 | ND2 | ASN | D | 204 | 29.121 | 21.712 | −51.525 | 1.00 | 73.83 | D |
| ATOM | 12461 | C | ASN | D | 204 | 32.866 | 22.613 | −53.051 | 1.00 | 45.41 | D |
| ATOM | 12462 | O | ASN | D | 204 | 33.567 | 21.951 | −52.284 | 1.00 | 45.41 | D |
| ATOM | 12463 | N | ILE | D | 205 | 33.061 | 23.910 | −53.256 | 1.00 | 44.71 | D |
| ATOM | 12464 | CA | ILE | D | 205 | 34.149 | 24.618 | −52.594 | 1.00 | 44.71 | D |
| ATOM | 12465 | CB | ILE | D | 205 | 34.187 | 26.102 | −53.020 | 1.00 | 45.67 | D |
| ATOM | 12466 | CG2 | ILE | D | 205 | 33.180 | 26.901 | −52.227 | 1.00 | 45.67 | D |
| ATOM | 12467 | CG1 | ILE | D | 205 | 35.583 | 26.671 | −52.786 | 1.00 | 45.67 | D |
| ATOM | 12468 | CD1 | ILE | D | 205 | 35.858 | 27.920 | −53.580 | 1.00 | 45.67 | D |
| ATOM | 12469 | C | ILE | D | 205 | 34.116 | 24.534 | −51.076 | 1.00 | 44.71 | D |
| ATOM | 12470 | O | ILE | D | 205 | 35.110 | 24.818 | −50.419 | 1.00 | 44.71 | D |
| ATOM | 12471 | N | SER | D | 206 | 32.975 | 24.151 | −50.518 | 1.00 | 61.32 | D |
| ATOM | 12472 | CA | SER | D | 206 | 32.855 | 24.024 | −49.071 | 1.00 | 61.32 | D |
| ATOM | 12473 | CB | SER | D | 206 | 31.386 | 23.972 | −48.664 | 1.00 | 69.12 | D |
| ATOM | 12474 | OG | SER | D | 206 | 30.751 | 25.198 | −48.974 | 1.00 | 69.12 | D |
| ATOM | 12475 | C | SER | D | 206 | 33.561 | 22.765 | −48.593 | 1.00 | 61.32 | D |
| ATOM | 12476 | O | SER | D | 206 | 33.925 | 22.653 | −47.430 | 1.00 | 61.32 | D |
| ATOM | 12477 | N | ARG | D | 207 | 33.748 | 21.815 | −49.502 | 1.00 | 54.39 | D |
| ATOM | 12478 | CA | ARG | D | 207 | 34.418 | 20.576 | −49.170 | 1.00 | 54.39 | D |
| ATOM | 12479 | CB | ARG | D | 207 | 33.808 | 19.422 | −49.955 | 1.00 | 57.41 | D |
| ATOM | 12480 | CG | ARG | D | 207 | 32.455 | 18.996 | −49.430 | 1.00 | 57.41 | D |
| ATOM | 12481 | CD | ARG | D | 207 | 32.233 | 17.514 | −49.638 | 1.00 | 57.41 | D |
| ATOM | 12482 | NE | ARG | D | 207 | 31.910 | 17.182 | −51.024 | 1.00 | 57.41 | D |
| ATOM | 12483 | CZ | ARG | D | 207 | 32.149 | 15.996 | −51.587 | 1.00 | 57.41 | D |
| ATOM | 12484 | NH1 | ARG | D | 207 | 32.726 | 15.025 | −50.887 | 1.00 | 57.41 | D |
| ATOM | 12485 | NH2 | ARG | D | 207 | 31.796 | 15.768 | −52.847 | 1.00 | 57.41 | D |
| ATOM | 12486 | C | ARG | D | 207 | 35.922 | 20.650 | −49.428 | 1.00 | 54.39 | D |
| ATOM | 12487 | O | ARG | D | 207 | 36.614 | 19.635 | −49.372 | 1.00 | 54.39 | D |
| ATOM | 12488 | N | SER | D | 208 | 36.422 | 21.851 | −49.711 | 1.00 | 47.80 | D |
| ATOM | 12489 | CA | SER | D | 208 | 37.843 | 22.038 | −49.957 | 1.00 | 47.80 | D |
| ATOM | 12490 | CB | SER | D | 208 | 38.099 | 22.364 | −51.419 | 1.00 | 56.84 | D |
| ATOM | 12491 | OG | SER | D | 208 | 39.497 | 22.448 | −51.646 | 1.00 | 56.84 | D |
| ATOM | 12492 | C | SER | D | 208 | 38.390 | 23.159 | −49.088 | 1.00 | 47.80 | D |
| ATOM | 12493 | O | SER | D | 208 | 38.710 | 24.240 | −49.573 | 1.00 | 47.80 | D |
| ATOM | 12494 | N | PRO | D | 209 | 38.506 | 22.901 | −47.783 | 1.00 | 42.06 | D |
| ATOM | 12495 | CD | PRO | D | 209 | 38.215 | 21.575 | −47.219 | 1.00 | 41.45 | D |
| ATOM | 12496 | CA | PRO | D | 209 | 38.997 | 23.806 | −46.742 | 1.00 | 42.06 | D |
| ATOM | 12497 | CB | PRO | D | 209 | 39.194 | 22.883 | −45.552 | 1.00 | 41.45 | D |
| ATOM | 12498 | CG | PRO | D | 209 | 38.128 | 21.877 | −45.748 | 1.00 | 41.45 | D |
| ATOM | 12499 | C | PRO | D | 209 | 40.283 | 24.525 | −47.092 | 1.00 | 42.06 | D |
| ATOM | 12500 | O | PRO | D | 209 | 40.515 | 25.637 | −46.624 | 1.00 | 42.06 | D |
| ATOM | 12501 | N | HIS | D | 210 | 41.114 | 23.902 | −47.923 | 1.00 | 56.59 | D |
| ATOM | 12502 | CA | HIS | D | 210 | 42.392 | 24.498 | −48.269 | 1.00 | 56.59 | D |
| ATOM | 12503 | CB | HIS | D | 210 | 43.457 | 23.412 | −48.227 | 1.00 | 59.44 | D |
| ATOM | 12504 | CG | HIS | D | 210 | 43.624 | 22.812 | −46.865 | 1.00 | 59.44 | D |
| ATOM | 12505 | CD2 | HIS | D | 210 | 43.250 | 21.609 | −46.366 | 1.00 | 59.44 | D |
| ATOM | 12506 | ND1 | HIS | D | 210 | 44.175 | 23.508 | −45.810 | 1.00 | 59.44 | D |
| ATOM | 12507 | CE1 | HIS | D | 210 | 44.133 | 22.762 | −44.720 | 1.00 | 59.44 | D |
| ATOM | 12508 | NE2 | HIS | D | 210 | 43.575 | 21.605 | −45.030 | 1.00 | 59.44 | D |
| ATOM | 12509 | C | HIS | D | 210 | 42.454 | 25.308 | −49.561 | 1.00 | 56.59 | D |
| ATOM | 12510 | O | HIS | D | 210 | 43.524 | 25.743 | −49.986 | 1.00 | 56.59 | D |
| ATOM | 12511 | N | THR | D | 211 | 41.303 | 25.516 | −50.186 | 1.00 | 40.27 | D |
| ATOM | 12512 | CA | THR | D | 211 | 41.243 | 26.342 | −51.375 | 1.00 | 40.27 | D |
| ATOM | 12513 | CB | THR | D | 211 | 40.021 | 26.005 | −52.238 | 1.00 | 50.51 | D |
| ATOM | 12514 | OG1 | THR | D | 211 | 40.121 | 24.649 | −52.691 | 1.00 | 50.51 | D |
| ATOM | 12515 | CG2 | THR | D | 211 | 39.949 | 26.935 | −53.439 | 1.00 | 50.51 | D |
| ATOM | 12516 | C | THR | D | 211 | 41.051 | 27.716 | −50.757 | 1.00 | 40.27 | D |
| ATOM | 12517 | O | THA | D | 211 | 40.146 | 27.903 | −49.938 | 1.00 | 40.27 | D |
| ATOM | 12518 | N | PRO | D | 212 | 41.901 | 28.690 | −51.119 | 1.00 | 49.43 | D |
| ATOM | 12519 | CD | PRO | D | 212 | 42.946 | 28.558 | −52.147 | 1.00 | 47.28 | D |
| ATOM | 12520 | CA | PRO | D | 212 | 41.846 | 30.067 | −50.600 | 1.00 | 49.43 | D |
| ATOM | 12521 | CB | PRO | D | 212 | 42.797 | 30.815 | −51.522 | 1.00 | 47.28 | D |
| ATOM | 12522 | CG | PRO | D | 212 | 43.799 | 29.768 | −51.878 | 1.00 | 47.28 | D |
| ATOM | 12523 | C | PRO | D | 212 | 40.449 | 30.709 | −50.569 | 1.00 | 49.43 | D |
| ATOM | 12524 | O | PRO | D | 212 | 39.662 | 30.550 | −51.505 | 1.00 | 49.43 | D |
| ATOM | 12525 | N | GLY | D | 213 | 40.148 | 31.439 | −49.496 | 1.00 | 42.92 | D |
| ATOM | 12526 | CA | GLY | D | 213 | 38.852 | 32.095 | −49.369 | 1.00 | 42.92 | D |
| ATOM | 12527 | C | GLY | D | 213 | 38.814 | 33.432 | −50.079 | 1.00 | 42.92 | D |
| ATOM | 12528 | O | GLY | D | 213 | 39.813 | 33.839 | −50.675 | 1.00 | 42.92 | D |
| ATOM | 12529 | N | ASN | D | 214 | 37.680 | 34.126 | −50.016 | 1.00 | 50.18 | D |
| ATOM | 12530 | CA | ASN | D | 214 | 37.516 | 35.435 | −50.684 | 1.00 | 50.18 | D |
| ATOM | 12531 | CB | ASN | D | 214 | 38.513 | 36.476 | −50.186 | 1.00 | 150.26 | D |
| ATOM | 12532 | CG | ASN | D | 214 | 38.804 | 36.317 | −48.760 | 1.00 | 144.43 | D |
| ATOM | 12533 | OD1 | ASN | D | 214 | 38.093 | 35.583 | −48.083 | 1.00 | 144.43 | D |
| ATOM | 12534 | ND2 | ASN | D | 214 | 39.826 | 37.005 | −48.259 | 1.00 | 144.43 | D |
| ATOM | 12535 | C | ASN | D | 214 | 37.780 | 35.247 | −52.154 | 1.00 | 50.18 | D |
| ATOM | 12536 | O | ASN | D | 214 | 38.570 | 35.981 | −52.744 | 1.00 | 50.18 | D |
| ATOM | 12537 | N | ASP | D | 215 | 37.126 | 34.260 | −52.741 | 1.00 | 41.09 | D |
| ATOM | 12538 | CA | ASP | D | 215 | 37.321 | 33.972 | −54.149 | 1.00 | 41.09 | D |
| ATOM | 12539 | CB | ASP | D | 215 | 36.573 | 34.987 | −54.996 | 1.00 | 158.59 | D |
| ATOM | 12540 | CG | ASP | D | 215 | 35.113 | 35.043 | −54.651 | 1.00 | 79.97 | D |
| ATOM | 12541 | OD1 | ASP | D | 215 | 34.455 | 33.979 | −54.702 | 1.00 | 79.97 | D |
| ATOM | 12542 | OD2 | ASP | D | 215 | 34.624 | 36.143 | −54.321 | 1.00 | 79.97 | D |
| ATOM | 12543 | C | ASP | D | 215 | 38.804 | 34.002 | −54.471 | 1.00 | 41.09 | D |
| ATOM | 12544 | O | ASP | D | 215 | 39.259 | 34.845 | −55.233 | 1.00 | 41.09 | D |
| ATOM | 12545 | N | TYR | D | 216 | 39.545 | 33.086 | −53.848 | 1.00 | 35.96 | D |
| ATOM | 12546 | CA | TYR | D | 216 | 40.985 | 32.942 | −54.047 | 1.00 | 35.96 | D |
| ATOM | 12547 | CB | TYR | D | 216 | 41.259 | 32.424 | −55.461 | 1.00 | 57.08 | D |
| ATOM | 12548 | CG | TYR | D | 216 | 40.490 | 31.174 | −55.813 | 1.00 | 35.83 | D |
| ATOM | 12549 | CD1 | TYR | D | 216 | 39.114 | 31.205 | −55.999 | 1.00 | 35.83 | D |
| ATOM | 12550 | CE1 | TYR | D | 216 | 38.396 | 30.055 | −56.320 | 1.00 | 35.83 | D |
| ATOM | 12551 | CD2 | TYR | D | 216 | 41.140 | 29.949 | −55.957 | 1.00 | 35.83 | D |
| ATOM | 12552 | CE2 | TYR | D | 216 | 40.433 | 28.790 | −56.283 | 1.00 | 35.83 | D |
| ATOM | 12553 | CZ | TYR | D | 216 | 39.061 | 28.860 | −56.463 | 1.00 | 35.83 | D |
| ATOM | 12554 | OH | TYR | D | 216 | 38.365 | 27.734 | −56.815 | 1.00 | 35.83 | D |
| ATOM | 12555 | C | TYR | D | 216 | 41.783 | 34.221 | −53.810 | 1.00 | 35.96 | D |
| ATOM | 12556 | O | TYR | D | 216 | 42.808 | 34.449 | −54.462 | 1.00 | 35.96 | D |
| ATOM | 12557 | N | ASN | D | 217 | 41.332 | 35.042 | −52.865 | 1.00 | 35.22 | D |
| ATOM | 12558 | CA | ASN | D | 217 | 42.012 | 36.298 | −52.579 | 1.00 | 35.22 | D |
| ATOM | 12559 | CB | ASN | D | 217 | 43.379 | 36.041 | −51.948 | 1.00 | 46.36 | D |
| ATOM | 12560 | CG | ASN | D | 217 | 43.282 | 35.284 | −50.644 | 1.00 | 41.09 | D |
| ATOM | 12561 | OD1 | ASN | D | 217 | 43.881 | 34.225 | −50.488 | 1.00 | 41.09 | D |
| ATOM | 12562 | ND2 | ASN | D | 217 | 42.528 | 35.825 | −49.698 | 1.00 | 41.09 | D |
| ATOM | 12563 | C | ASN | D | 217 | 42.200 | 37.073 | −53.875 | 1.00 | 35.22 | D |
| ATOM | 12564 | O | ASN | D | 217 | 43.226 | 37.726 | −54.071 | 1.00 | 35.22 | D |
| ATOM | 12565 | N | PHE | D | 218 | 41.204 | 37.004 | −54.756 | 1.00 | 38.93 | D |
| ATOM | 12566 | CA | PHE | D | 218 | 41.290 | 37.684 | −56.036 | 1.00 | 38.93 | D |
| ATOM | 12567 | CB | PHE | D | 218 | 40.079 | 37.392 | −56.897 | 1.00 | 39.26 | D |
| ATOM | 12568 | CG | PHE | D | 218 | 40.175 | 38.002 | −58.244 | 1.00 | 35.78 | D |
| ATOM | 12569 | CD1 | PHE | D | 218 | 40.853 | 37.348 | −59.256 | 1.00 | 35.78 | D |
| ATOM | 12570 | CD2 | PHE | D | 218 | 39.636 | 39.260 | −58.495 | 1.00 | 35.78 | D |
| ATOM | 12571 | CE1 | PHE | D | 218 | 41.002 | 37.931 | −60.521 | 1.00 | 35.78 | D |
| ATOM | 12572 | CE2 | PHE | D | 218 | 39.775 | 39.850 | −59.737 | 1.00 | 35.78 | D |
| ATOM | 12573 | CZ | PHE | D | 218 | 40.463 | 39.179 | −60.759 | 1.00 | 35.78 | D |
| ATOM | 12574 | C | PHE | D | 218 | 41.448 | 39.196 | −55.941 | 1.00 | 38.93 | D |
| ATOM | 12575 | O | PHE | D | 218 | 42.332 | 39.769 | −56.580 | 1.00 | 38.93 | D |
| ATOM | 12576 | N | ASN | D | 219 | 40.589 | 39.847 | −55.166 | 1.00 | 44.93 | D |
| ATOM | 12577 | CA | ASN | D | 219 | 40.701 | 41.290 | −55.047 | 1.00 | 44.93 | D |
| ATOM | 12578 | CB | ASN | D | 219 | 39.653 | 41.836 | −54.088 | 1.00 | 45.54 | D |
| ATOM | 12579 | CG | ASN | D | 219 | 38.254 | 41.495 | −54.516 | 1.00 | 37.17 | D |
| ATOM | 12580 | OD1 | ASN | D | 219 | 37.940 | 41.481 | −55.698 | 1.00 | 37.17 | D |
| ATOM | 12581 | ND2 | ASN | D | 219 | 37.398 | 41.226 | −53.551 | 1.00 | 37.17 | D |
| ATOM | 12582 | C | ASN | D | 219 | 42.101 | 41.679 | −54.583 | 1.00 | 44.93 | D |
| ATOM | 12583 | O | ASN | D | 219 | 42.707 | 42.617 | −55.114 | 1.00 | 44.93 | D |
| ATOM | 12584 | N | PHE | D | 220 | 42.617 | 40.952 | −53.596 | 1.00 | 37.66 | D |
| ATOM | 12585 | CA | PHE | D | 220 | 43.956 | 41.223 | −53.091 | 1.00 | 37.66 | D |
| ATOM | 12586 | CB | PHE | D | 220 | 44.333 | 40.231 | −52.010 | 1.00 | 44.26 | D |
| ATOM | 12587 | CG | PHE | D | 220 | 45.792 | 40.258 | −51.666 | 1.00 | 37.24 | D |
| ATOM | 12588 | CD1 | PHE | D | 220 | 46.289 | 41.186 | −50.761 | 1.00 | 37.24 | D |
| ATOM | 12589 | CD2 | PHE | D | 220 | 46.684 | 39.375 | −52.287 | 1.00 | 37.24 | D |
| ATOM | 12590 | CE1 | PHE | D | 220 | 47.647 | 41.246 | −50.476 | 1.00 | 37.24 | D |
| ATOM | 12591 | CE2 | PHE | D | 220 | 48.051 | 39.424 | −52.010 | 1.00 | 37.24 | D |
| ATOM | 12592 | CZ | PHE | D | 220 | 48.532 | 40.365 | −51.101 | 1.00 | 37.24 | D |
| ATOM | 12593 | C | PHE | D | 220 | 45.002 | 41.144 | −54.194 | 1.00 | 37.66 | D |
| ATOM | 12594 | O | PHE | D | 220 | 45.764 | 42.075 | −54.401 | 1.00 | 37.66 | D |
| ATOM | 12595 | N | TRP | D | 221 | 45.044 | 40.021 | −54.895 | 1.00 | 37.42 | D |
| ATOM | 12596 | CA | TRP | D | 221 | 46.014 | 39.842 | −55.966 | 1.00 | 37.42 | D |
| ATOM | 12597 | CB | TRP | D | 221 | 45.990 | 38.388 | −56.461 | 1.00 | 47.13 | D |
| ATOM | 12598 | CG | TRP | D | 221 | 46.606 | 37.431 | −55.485 | 1.00 | 40.28 | D |
| ATOM | 12599 | CD2 | TRP | D | 221 | 47.963 | 37.437 | −55.031 | 1.00 | 40.28 | D |
| ATOM | 12600 | CE2 | TRP | D | 221 | 48.090 | 36.407 | −54.080 | 1.00 | 40.28 | D |
| ATOM | 12601 | CE3 | TRP | D | 221 | 49.086 | 38.218 | −55.338 | 1.00 | 40.28 | D |
| ATOM | 12602 | CD1 | TRP | D | 221 | 45.982 | 36.422 | −54.811 | 1.00 | 40.28 | D |
| ATOM | 12603 | NE1 | TRP | D | 221 | 46.866 | 35.804 | −53.963 | 1.00 | 40.28 | D |
| ATOM | 12604 | CZ2 | TRP | D | 221 | 49.297 | 36.133 | −53.429 | 1.00 | 40.28 | D |
| ATOM | 12605 | CZ3 | TRP | D | 221 | 50.290 | 37.943 | −54.689 | 1.00 | 40.28 | D |
| ATOM | 12606 | CH2 | TRP | D | 221 | 50.383 | 36.909 | −53.744 | 1.00 | 40.28 | D |
| ATOM | 12607 | C | TRP | D | 221 | 45.820 | 40.802 | −57.143 | 1.00 | 37.42 | D |
| ATOM | 12608 | O | TRP | D | 221 | 46.776 | 41.423 | −57.623 | 1.00 | 37.42 | D |
| ATOM | 12609 | N | TYR | D | 222 | 44.587 | 40.934 | −57.613 | 1.00 | 44.04 | D |
| ATOM | 12610 | CA | TYR | D | 222 | 44.335 | 41.816 | −58.740 | 1.00 | 44.04 | D |
| ATOM | 12611 | CB | TYR | D | 222 | 42.832 | 41.993 | −58.958 | 1.00 | 40.57 | D |
| ATOM | 12612 | CG | TYR | D | 222 | 42.536 | 43.079 | −59.949 | 1.00 | 39.14 | D |
| ATOM | 12613 | CD1 | TYR | D | 222 | 42.697 | 42.856 | −61.314 | 1.00 | 39.14 | D |
| ATOM | 12614 | CE1 | TYR | D | 222 | 42.525 | 43.881 | −62.228 | 1.00 | 39.14 | D |
| ATOM | 12615 | CD2 | TYR | D | 222 | 42.186 | 44.358 | −59.518 | 1.00 | 39.14 | D |
| ATOM | 12616 | CE2 | TYR | D | 222 | 42.015 | 45.392 | −60.425 | 1.00 | 39.14 | D |
| ATOM | 12617 | CZ | TYR | D | 222 | 42.187 | 45.146 | −61.777 | 1.00 | 39.14 | D |
| ATOM | 12618 | OH | TYR | D | 222 | 42.039 | 46.161 | −62.683 | 1.00 | 39.14 | D |
| ATOM | 12619 | C | TYR | D | 222 | 44.966 | 43.175 | −58.464 | 1.00 | 44.04 | D |
| ATOM | 12620 | O | TYR | D | 222 | 45.753 | 43.709 | −59.245 | 1.00 | 44.04 | D |
| ATOM | 12621 | N | SER | D | 223 | 44.606 | 43.722 | −57.321 | 1.00 | 43.13 | D |
| ATOM | 12622 | CA | SER | D | 223 | 45.096 | 45.013 | −56.927 | 1.00 | 43.13 | D |
| ATOM | 12623 | CB | SER | D | 223 | 44.447 | 45.407 | −55.620 | 1.00 | 36.60 | D |
| ATOM | 12624 | OG | SER | D | 223 | 44.979 | 46.637 | −55.197 | 1.00 | 35.22 | D |
| ATOM | 12625 | C | SER | D | 223 | 46.613 | 45.097 | −56.808 | 1.00 | 43.13 | D |
| ATOM | 12626 | O | SER | D | 223 | 47.234 | 45.996 | −57.382 | 1.00 | 43.13 | D |
| ATOM | 12627 | N | LEU | D | 224 | 47.210 | 44.163 | −56.069 | 1.00 | 32.28 | D |
| ATOM | 12628 | CA | LEU | D | 224 | 48.653 | 44.156 | −55.879 | 1.00 | 32.28 | D |
| ATOM | 12629 | CB | LEU | D | 224 | 49.035 | 43.132 | −54.827 | 1.00 | 66.69 | D |
| ATOM | 12630 | CG | LEU | D | 224 | 50.533 | 43.073 | −54.545 | 1.00 | 43.93 | D |
| ATOM | 12631 | CD1 | LEU | D | 224 | 51.069 | 44.456 | −54.151 | 1.00 | 43.93 | D |
| ATOM | 12632 | CD2 | LEU | D | 224 | 50.776 | 42.071 | −53.431 | 1.00 | 43.93 | D |
| ATOM | 12633 | C | LEU | D | 224 | 49.412 | 43.876 | −57.169 | 1.00 | 32.28 | D |
| ATOM | 12634 | O | LEU | D | 224 | 50.529 | 44.344 | −57.360 | 1.00 | 32.28 | D |
| ATOM | 12635 | N | MET | D | 225 | 48.806 | 43.112 | −58.066 | 1.00 | 43.93 | D |
| ATOM | 12636 | CA | MET | D | 225 | 49.459 | 42.822 | −59.329 | 1.00 | 43.93 | D |
| ATOM | 12637 | CB | MET | D | 225 | 48.827 | 41.604 | −59.979 | 1.00 | 76.38 | D |
| ATOM | 12638 | CG | MET | D | 225 | 49.120 | 40.335 | −59.227 | 1.00 | 45.10 | D |
| ATOM | 12639 | SD | MET | D | 225 | 48.529 | 38.924 | −60.114 | 1.00 | 45.10 | D |
| ATOM | 12640 | CE | MET | D | 225 | 49.558 | 38.970 | −61.568 | 1.00 | 45.10 | D |
| ATOM | 12641 | C | MET | D | 225 | 49.330 | 44.029 | −60.237 | 1.00 | 43.93 | D |
| ATOM | 12642 | O | MET | D | 225 | 50.226 | 44.339 | −61.021 | 1.00 | 43.93 | D |
| ATOM | 12643 | N | LYS | D | 226 | 48.200 | 44.716 | −60.125 | 1.00 | 56.86 | D |
| ATOM | 12644 | CA | LYS | D | 226 | 47.981 | 45.897 | −60.932 | 1.00 | 56.86 | D |
| ATOM | 12645 | CB | LYS | D | 226 | 46.616 | 46.498 | −60.626 | 1.00 | 103.21 | D |
| ATOM | 12646 | CG | LYS | D | 226 | 46.309 | 47.738 | −61.429 | 1.00 | 54.67 | D |
| ATOM | 12647 | CD | LYS | D | 226 | 45.016 | 48.360 | −60.960 | 1.00 | 54.67 | D |
| ATOM | 12648 | CE | LYS | D | 226 | 44.815 | 49.735 | −61.567 | 1.00 | 54.67 | D |
| ATOM | 12649 | NZ | LYS | D | 226 | 43.553 | 50.355 | −61.070 | 1.00 | 54.67 | D |
| ATOM | 12650 | C | LYS | D | 226 | 49.088 | 46.872 | −60.562 | 1.00 | 56.86 | D |
| ATOM | 12651 | O | LYS | D | 226 | 49.747 | 47.438 | −61.428 | 1.00 | 56.86 | D |
| ATOM | 12652 | N | GLU | D | 227 | 49.303 | 47.043 | −59.261 | 1.00 | 68.67 | D |
| ATOM | 12653 | CA | GLU | D | 227 | 50.337 | 47.944 | −58.762 | 1.00 | 68.67 | D |
| ATOM | 12654 | CB | GLU | D | 227 | 50.395 | 47.898 | −57.234 | 1.00 | 120.79 | D |
| ATOM | 12655 | CG | GLU | D | 227 | 51.361 | 48.901 | −56.632 | 1.00 | 96.59 | D |
| ATOM | 12656 | CD | GLU | D | 227 | 51.664 | 48.631 | −55.166 | 1.00 | 96.59 | D |
| ATOM | 12657 | OE1 | GLU | D | 227 | 50.712 | 48.499 | −54.363 | 1.00 | 96.59 | D |
| ATOM | 12658 | OE2 | GLU | D | 227 | 52.863 | 48.560 | −54.815 | 1.00 | 96.59 | D |
| ATOM | 12659 | C | GLU | D | 227 | 51.691 | 47.534 | −59.324 | 1.00 | 68.67 | D |
| ATOM | 12660 | O | GLU | D | 227 | 52.439 | 48.361 | −59.839 | 1.00 | 68.67 | D |
| ATOM | 12661 | N | THR | D | 228 | 51.996 | 46.245 | −59.230 | 1.00 | 51.96 | D |
| ATOM | 12662 | CA | THR | D | 228 | 53.266 | 45.727 | −59.715 | 1.00 | 51.96 | D |
| ATOM | 12663 | CB | THR | D | 228 | 53.388 | 44.231 | −59.428 | 1.00 | 57.77 | D |
| ATOM | 12664 | OG1 | THR | D | 228 | 53.268 | 44.015 | −58.018 | 1.00 | 43.76 | D |
| ATOM | 12665 | CG2 | THR | D | 228 | 54.736 | 43.704 | −59.922 | 1.00 | 43.76 | D |
| ATOM | 12666 | C | THR | D | 228 | 53.466 | 45.948 | −61.205 | 1.00 | 51.96 | D |
| ATOM | 12667 | O | THR | D | 228 | 54.567 | 46.258 | −61.653 | 1.00 | 51.96 | D |
| ATOM | 12668 | N | LEU | D | 229 | 52.401 | 45.777 | −61.978 | 1.00 | 61.19 | D |
| ATOM | 12669 | CA | LEU | D | 229 | 52.507 | 45.959 | −63.412 | 1.00 | 61.19 | D |
| ATOM | 12670 | CB | LEU | D | 229 | 51.217 | 45.510 | −64.091 | 1.00 | 65.97 | D |
| ATOM | 12671 | CG | LEU | D | 229 | 51.190 | 45.624 | −65.615 | 1.00 | 37.85 | D |
| ATOM | 12672 | CD1 | LEU | D | 229 | 52.494 | 45.111 | −66.209 | 1.00 | 37.85 | D |
| ATOM | 12673 | CD2 | LEU | D | 229 | 50.002 | 44.841 | −66.154 | 1.00 | 37.85 | D |
| ATOM | 12674 | C | LEU | D | 229 | 52.815 | 47.418 | −63.737 | 1.00 | 61.19 | D |
| ATOM | 12675 | O | LEU | D | 229 | 53.632 | 47.703 | −64.604 | 1.00 | 61.19 | D |
| ATOM | 12676 | N | GLU | D | 230 | 52.174 | 48.339 | −63.027 | 1.00 | 70.80 | D |
| ATOM | 12677 | CA | GLU | D | 230 | 52.402 | 49.761 | −63.246 | 1.00 | 70.80 | D |
| ATOM | 12678 | CB | GLU | D | 230 | 51.480 | 50.591 | −62.353 | 1.00 | 156.74 | D |
| ATOM | 12679 | CG | GLU | D | 230 | 50.020 | 50.519 | −62.768 | 1.00 | 121.20 | D |
| ATOM | 12680 | CD | GLU | D | 230 | 49.117 | 51.383 | −61.906 | 1.00 | 121.20 | D |
| ATOM | 12681 | OE1 | GLU | D | 230 | 49.425 | 52.581 | −61.743 | 1.00 | 121.20 | D |
| ATOM | 12682 | OE2 | GLU | D | 230 | 48.095 | 50.874 | −61.398 | 1.00 | 121.20 | D |
| ATOM | 12683 | C | GLU | D | 230 | 53.858 | 50.127 | −62.983 | 1.00 | 70.80 | D |
| ATOM | 12684 | O | GLU | D | 230 | 54.485 | 50.806 | −63.793 | 1.00 | 70.80 | D |
| ATOM | 12685 | N | GLU | D | 231 | 54.400 | 49.672 | −61.857 | 1.00 | 73.46 | D |
| ATOM | 12686 | CA | GLU | D | 231 | 55.794 | 49.947 | −61.509 | 1.00 | 73.46 | D |
| ATOM | 12687 | CB | GLU | D | 231 | 56.134 | 49.320 | −60.155 | 1.00 | 158.78 | D |
| ATOM | 12688 | CG | GLU | D | 231 | 55.219 | 49.747 | −59.025 | 1.00 | 135.02 | D |
| ATOM | 12689 | CD | GLU | D | 231 | 55.364 | 51.211 | −58.675 | 1.00 | 135.02 | D |
| ATOM | 12690 | OE1 | GLU | D | 231 | 55.229 | 52.062 | −59.581 | 1.00 | 135.02 | D |
| ATOM | 12691 | OE2 | GLU | D | 231 | 55.609 | 51.510 | −57.487 | 1.00 | 135.02 | D |
| ATOM | 12692 | C | GLU | D | 231 | 56.719 | 49.367 | −62.581 | 1.00 | 73.46 | D |
| ATOM | 12693 | O | GLU | D | 231 | 57.643 | 50.031 | −63.038 | 1.00 | 73.46 | D |
| ATOM | 12694 | N | ILE | D | 232 | 56.463 | 48.124 | −62.975 | 1.00 | 67.93 | D |
| ATOM | 12695 | CA | ILE | D | 232 | 57.267 | 47.460 | −63.989 | 1.00 | 67.93 | D |
| ATOM | 12696 | CB | ILE | D | 232 | 56.733 | 46.038 | −64.282 | 1.00 | 74.26 | D |
| ATOM | 12697 | CG2 | ILE | D | 232 | 57.353 | 45.479 | −65.548 | 1.00 | 44.31 | D |
| ATOM | 12698 | CG1 | ILE | D | 232 | 57.066 | 45.113 | −63.120 | 1.00 | 44.31 | D |
| ATOM | 12699 | CD1 | ILE | D | 232 | 56.547 | 43.690 | −63.305 | 1.00 | 44.31 | D |
| ATOM | 12700 | C | ILE | D | 232 | 57.297 | 48.243 | −65.294 | 1.00 | 67.93 | D |
| ATOM | 12701 | O | ILE | D | 232 | 58.305 | 48.230 | −65.995 | 1.00 | 67.93 | D |
| ATOM | 12702 | N | ASN | D | 233 | 56.202 | 48.925 | −65.623 | 1.00 | 73.28 | D |
| ATOM | 12703 | CA | ASN | D | 233 | 56.146 | 49.683 | −66.866 | 1.00 | 73.28 | D |
| ATOM | 12704 | CB | ASN | D | 233 | 54.703 | 49.839 | −67.334 | 1.00 | 83.28 | D |
| ATOM | 12705 | CG | ASN | D | 233 | 54.120 | 48.542 | −67.834 | 1.00 | 72.20 | D |
| ATOM | 12706 | OD1 | ASN | D | 233 | 54.832 | 47.708 | −68.381 | 1.00 | 72.20 | D |
| ATOM | 12707 | ND2 | ASN | D | 233 | 52.816 | 48.368 | −67.664 | 1.00 | 72.20 | D |
| ATOM | 12708 | C | ASN | D | 233 | 56.819 | 51.052 | −66.845 | 1.00 | 73.28 | D |
| ATOM | 12709 | O | ASN | D | 233 | 57.003 | 51.661 | −67.903 | 1.00 | 73.28 | D |
| ATOM | 12710 | N | LYS | D | 234 | 57.191 | 51.540 | −65.663 | 1.00 | 107.06 | D |
| ATOM | 12711 | CA | LYS | D | 234 | 57.851 | 52.839 | −65.575 | 1.00 | 107.06 | D |
| ATOM | 12712 | CB | LYS | D | 234 | 57.960 | 53.287 | −64.117 | 1.00 | 74.61 | D |
| ATOM | 12713 | CG | LYS | D | 234 | 56.621 | 53.751 | −63.573 | 1.00 | 74.61 | D |
| ATOM | 12714 | CD | LYS | D | 234 | 56.701 | 54.253 | −62.147 | 1.00 | 74.61 | D |
| ATOM | 12715 | CE | LYS | D | 234 | 55.328 | 54.738 | −61.686 | 1.00 | 74.61 | D |
| ATOM | 12716 | NZ | LYS | D | 234 | 55.307 | 55.091 | −60.238 | 1.00 | 74.61 | D |
| ATOM | 12717 | C | LYS | D | 234 | 59.218 | 52.813 | −66.259 | 1.00 | 107.06 | D |
| ATOM | 12718 | O | LYS | D | 234 | 59.546 | 53.726 | −67.017 | 1.00 | 107.06 | D |
| ATOM | 12719 | N | ASN | D | 235 | 60.020 | 51.785 | −65.993 | 1.00 | 93.68 | D |
| ATOM | 12720 | CA | ASN | D | 235 | 61.306 | 51.667 | −66.677 | 1.00 | 93.68 | D |
| ATOM | 12721 | CB | ASN | D | 235 | 62.215 | 50.636 | −65.993 | 1.00 | 99.29 | D |
| ATOM | 12722 | CG | ASN | D | 235 | 63.510 | 50.387 | −66.770 | 1.00 | 99.29 | D |
| ATOM | 12723 | OD1 | ASN | D | 235 | 63.490 | 50.274 | −67.995 | 1.00 | 99.29 | D |
| ATOM | 12724 | ND2 | ASN | D | 235 | 64.627 | 50.275 | −66.054 | 1.00 | 99.29 | D |
| ATOM | 12725 | C | ASN | D | 235 | 60.882 | 51.159 | −68.053 | 1.00 | 93.68 | D |
| ATOM | 12726 | O | ASN | D | 235 | 60.460 | 50.017 | −68.193 | 1.00 | 93.68 | D |
| ATOM | 12727 | N | ASP | D | 236 | 60.983 | 52.011 | −69.063 | 1.00 | 124.48 | D |
| ATOM | 12728 | CA | ASP | D | 236 | 60.559 | 51.652 | −70.411 | 1.00 | 124.48 | D |
| ATOM | 12729 | CB | ASP | D | 236 | 60.716 | 52.868 | −71.330 | 1.00 | 135.31 | D |
| ATOM | 12730 | CG | ASP | D | 236 | 59.780 | 52.822 | −72.523 | 1.00 | 135.31 | D |
| ATOM | 12731 | OD1 | ASP | D | 236 | 59.995 | 51.986 | −73.425 | 1.00 | 135.31 | D |
| ATOM | 12732 | OD2 | ASP | D | 236 | 58.820 | 53.622 | −72.554 | 1.00 | 135.31 | D |
| ATOM | 12733 | C | ASP | D | 236 | 61.239 | 50.429 | −71.044 | 1.00 | 124.48 | D |
| ATOM | 12734 | O | ASP | D | 236 | 60.841 | 49.988 | −72.122 | 1.00 | 124.48 | D |
| ATOM | 12735 | N | SER | D | 237 | 62.251 | 49.873 | −70.385 | 1.00 | 93.61 | D |
| ATOM | 12736 | CA | SER | D | 237 | 62.940 | 48.707 | −70.936 | 1.00 | 93.61 | D |
| ATOM | 12737 | CB | SER | D | 237 | 64.438 | 48.767 | −70.615 | 1.00 | 112.89 | D |
| ATOM | 12738 | OG | SER | D | 237 | 64.688 | 48.461 | −69.254 | 1.00 | 112.89 | D |
| ATOM | 12739 | C | SER | D | 237 | 62.357 | 47.401 | −70.397 | 1.00 | 93.61 | D |
| ATOM | 12740 | O | SER | D | 237 | 62.858 | 46.315 | −70.698 | 1.00 | 93.61 | D |
| ATOM | 12741 | N | THR | D | 238 | 61.299 | 47.514 | −69.597 | 1.00 | 101.49 | D |
| ATOM | 12742 | CA | THR | D | 238 | 60.642 | 46.346 | −69.015 | 1.00 | 101.49 | D |
| ATOM | 12743 | CB | THR | D | 238 | 60.677 | 46.382 | −67.467 | 1.00 | 104.94 | D |
| ATOM | 12744 | OG1 | THR | D | 238 | 61.991 | 46.740 | −67.025 | 1.00 | 104.94 | D |
| ATOM | 12745 | CG2 | THR | D | 238 | 60.321 | 45.014 | −66.889 | 1.00 | 104.94 | D |
| ATOM | 12746 | C | THR | D | 238 | 59.185 | 46.308 | −69.450 | 1.00 | 101.49 | D |
| ATOM | 12747 | O | THR | D | 238 | 58.576 | 45.243 | −69.491 | 1.00 | 101.49 | D |
| ATOM | 12748 | N | LYS | D | 239 | 58.643 | 47.480 | −69.771 | 1.00 | 92.00 | D |
| ATOM | 12749 | CA | LYS | D | 239 | 57.256 | 47.629 | −70.199 | 1.00 | 92.00 | D |
| ATOM | 12750 | CB | LYS | D | 239 | 57.154 | 48.741 | −71.246 | 1.00 | 98.70 | D |
| ATOM | 12751 | CG | LYS | D | 239 | 55.735 | 49.036 | −71.703 | 1.00 | 98.70 | D |
| ATOM | 12752 | CD | LYS | D | 239 | 55.678 | 50.303 | −72.540 | 1.00 | 98.70 | D |
| ATOM | 12753 | CE | LYS | D | 239 | 56.086 | 51.518 | −71.722 | 1.00 | 98.70 | D |
| ATOM | 12754 | NZ | LYS | D | 239 | 56.027 | 52.767 | −72.527 | 1.00 | 98.70 | D |
| ATOM | 12755 | C | LYS | D | 239 | 56.672 | 46.328 | −70.748 | 1.00 | 92.00 | D |
| ATOM | 12756 | O | LYS | D | 239 | 57.204 | 45.746 | −71.696 | 1.00 | 92.00 | D |
| ATOM | 12757 | N | THR | D | 240 | 55.577 | 45.876 | −70.137 | 1.00 | 71.70 | D |
| ATOM | 12758 | CA | THR | D | 240 | 54.925 | 44.637 | −70.539 | 1.00 | 71.70 | D |
| ATOM | 12759 | CB | THR | D | 240 | 55.198 | 43.508 | −69.528 | 1.00 | 106.40 | D |
| ATOM | 12760 | OG1 | THR | D | 240 | 56.609 | 43.376 | −69.325 | 1.00 | 106.40 | D |
| ATOM | 12761 | CG2 | THR | D | 240 | 54.643 | 42.186 | −70.046 | 1.00 | 106.40 | D |
| ATOM | 12762 | C | THR | D | 240 | 53.417 | 44.789 | −70.661 | 1.00 | 71.70 | D |
| ATOM | 12763 | O | THR | D | 240 | 52.808 | 45.649 | −70.024 | 1.00 | 71.70 | D |
| ATOM | 12764 | N | THR | D | 241 | 52.833 | 43.927 | −71.485 | 1.00 | 59.48 | D |
| ATOM | 12765 | CA | THR | D | 241 | 51.401 | 43.899 | −71.729 | 1.00 | 59.48 | D |
| ATOM | 12766 | CB | THR | D | 241 | 51.070 | 42.950 | −72.886 | 1.00 | 106.88 | D |
| ATOM | 12767 | OG1 | THR | D | 241 | 51.925 | 43.233 | −73.998 | 1.00 | 66.58 | D |
| ATOM | 12768 | CG2 | THR | D | 241 | 49.624 | 43.106 | −73.300 | 1.00 | 66.58 | D |
| ATOM | 12769 | C | THR | D | 241 | 50.663 | 43.368 | −70.501 | 1.00 | 59.48 | D |
| ATOM | 12770 | O | THR | D | 241 | 49.629 | 43.901 | −70.101 | 1.00 | 59.48 | D |
| ATOM | 12771 | N | SER | D | 242 | 51.200 | 42.309 | −69.902 | 1.00 | 57.44 | D |
| ATOM | 12772 | CA | SER | D | 242 | 50.555 | 41.698 | −68.756 | 1.00 | 57.44 | D |
| ATOM | 12773 | CB | SER | D | 242 | 49.594 | 40.617 | −69.242 | 1.00 | 82.15 | D |
| ATOM | 12774 | OG | SER | D | 242 | 50.308 | 39.610 | −69.945 | 1.00 | 54.67 | D |
| ATOM | 12775 | C | SER | D | 242 | 51.515 | 41.077 | −67.755 | 1.00 | 57.44 | D |
| ATOM | 12776 | O | SER | D | 242 | 52.712 | 40.940 | −68.009 | 1.00 | 57.44 | D |
| ATOM | 12777 | N | LEU | D | 243 | 50.944 | 40.679 | −66.624 | 1.00 | 54.94 | D |
| ATOM | 12778 | CA | LEU | D | 243 | 51.673 | 40.051 | −65.540 | 1.00 | 54.94 | D |
| ATOM | 12779 | CB | LEU | D | 243 | 51.866 | 41.066 | −64.418 | 1.00 | 62.63 | D |
| ATOM | 12780 | CG | LEU | D | 243 | 52.838 | 40.674 | −63.310 | 1.00 | 38.76 | D |
| ATOM | 12781 | CD1 | LEU | D | 243 | 54.204 | 40.409 | −63.912 | 1.00 | 38.76 | D |
| ATOM | 12782 | CD2 | LEU | D | 243 | 52.909 | 41.786 | −62.280 | 1.00 | 38.76 | D |
| ATOM | 12783 | C | LEU | D | 243 | 50.855 | 38.847 | −65.044 | 1.00 | 54.94 | D |
| ATOM | 12784 | O | LEU | D | 243 | 49.627 | 38.921 | −64.945 | 1.00 | 54.94 | D |
| ATOM | 12785 | N | SER | D | 244 | 51.533 | 37.745 | −64.738 | 1.00 | 41.69 | D |
| ATOM | 12786 | CA | SER | D | 244 | 50.852 | 36.547 | −64.265 | 1.00 | 41.69 | D |
| ATOM | 12787 | CB | SER | D | 244 | 50.833 | 35.472 | −65.356 | 1.00 | 40.73 | D |
| ATOM | 12788 | OG | SER | D | 244 | 50.020 | 35.836 | −66.450 | 1.00 | 42.88 | D |
| ATOM | 12789 | C | SER | D | 244 | 51.473 | 35.933 | −63.026 | 1.00 | 41.69 | D |
| ATOM | 12790 | O | SER | D | 244 | 52.694 | 35.900 | −62.891 | 1.00 | 41.69 | D |
| ATOM | 12791 | N | LEU | D | 245 | 50.617 | 35.447 | −62.128 | 1.00 | 55.67 | D |
| ATOM | 12792 | CA | LEU | D | 245 | 51.064 | 34.770 | −60.925 | 1.00 | 55.67 | D |
| ATOM | 12793 | CB | LEU | D | 245 | 50.375 | 35.340 | −59.694 | 1.00 | 56.98 | D |
| ATOM | 12794 | CG | LEU | D | 245 | 50.743 | 34.586 | −58.416 | 1.00 | 40.97 | D |
| ATOM | 12795 | CD1 | LEU | D | 245 | 52.244 | 34.611 | −58.217 | 1.00 | 40.97 | D |
| ATOM | 12796 | CD2 | LEU | D | 245 | 50.029 | 35.212 | −57.228 | 1.00 | 40.97 | D |
| ATOM | 12797 | C | LEU | D | 245 | 50.688 | 33.295 | −61.115 | 1.00 | 55.67 | D |
| ATOM | 12798 | O | LEU | D | 245 | 49.512 | 32.936 | −61.098 | 1.00 | 55.67 | D |
| ATOM | 12799 | N | ILE | D | 246 | 51.691 | 32.450 | −61.336 | 1.00 | 36.75 | D |
| ATOM | 12800 | CA | ILE | D | 246 | 51.471 | 31.024 | −61.543 | 1.00 | 36.75 | D |
| ATOM | 12801 | CB | ILE | D | 246 | 52.313 | 30.504 | −62.716 | 1.00 | 46.51 | D |
| ATOM | 12802 | CG2 | ILE | D | 246 | 51.994 | 29.051 | −62.986 | 1.00 | 29.55 | D |
| ATOM | 12803 | CG1 | ILE | D | 246 | 52.023 | 31.328 | −63.973 | 1.00 | 29.55 | D |
| ATOM | 12804 | CD1 | ILE | D | 246 | 50.579 | 31.358 | −64.358 | 1.00 | 29.55 | D |
| ATOM | 12805 | C | ILE | D | 246 | 51.863 | 30.255 | −60.296 | 1.00 | 36.75 | D |
| ATOM | 12806 | O | ILE | D | 246 | 53.020 | 30.283 | −59.884 | 1.00 | 36.75 | D |
| ATOM | 12807 | N | THR | D | 247 | 50.903 | 29.573 | −59.688 | 1.00 | 34.40 | D |
| ATOM | 12808 | CA | THR | D | 247 | 51.194 | 28.812 | −58.487 | 1.00 | 34.40 | D |
| ATOM | 12809 | CB | THR | D | 247 | 50.901 | 29.643 | −57.235 | 1.00 | 54.08 | D |
| ATOM | 12810 | OG1 | THR | D | 247 | 51.400 | 28.956 | −56.089 | 1.00 | 51.32 | D |
| ATOM | 12811 | CG2 | THR | D | 247 | 49.423 | 29.855 | −57.063 | 1.00 | 51.32 | D |
| ATOM | 12812 | C | THR | D | 247 | 50.360 | 27.534 | −58.496 | 1.00 | 34.40 | D |
| ATOM | 12813 | O | THR | D | 247 | 49.281 | 27.497 | −59.080 | 1.00 | 34.40 | D |
| ATOM | 12814 | N | GLY | D | 248 | 50.872 | 26.478 | −57.881 | 1.00 | 43.52 | D |
| ATOM | 12815 | CA | GLY | D | 248 | 50.145 | 25.225 | −57.867 | 1.00 | 43.52 | D |
| ATOM | 12816 | C | GLY | D | 248 | 51.037 | 24.069 | −57.472 | 1.00 | 43.52 | D |
| ATOM | 12817 | O | GLY | D | 248 | 52.178 | 24.275 | −57.079 | 1.00 | 43.52 | D |
| ATOM | 12818 | N | CYS | D | 249 | 50.530 | 22.848 | −57.574 | 1.00 | 39.77 | D |
| ATOM | 12819 | CA | CYS | D | 249 | 51.324 | 21.691 | −57.200 | 1.00 | 39.77 | D |
| ATOM | 12820 | CB | CYS | D | 249 | 51.075 | 21.336 | −55.735 | 1.00 | 29.13 | D |
| ATOM | 12821 | SG | CYS | D | 249 | 49.491 | 20.598 | −55.443 | 1.00 | 47.90 | D |
| ATOM | 12822 | C | CYS | D | 249 | 51.019 | 20.490 | −58.087 | 1.00 | 39.77 | D |
| ATOM | 12823 | O | CYS | D | 249 | 50.186 | 20.569 | −58.987 | 1.00 | 39.77 | D |
| ATOM | 12824 | N | TYR | D | 250 | 51.696 | 19.379 | −57.835 | 1.00 | 44.22 | D |
| ATOM | 12825 | CA | TYR | D | 250 | 51.492 | 18.190 | −58.644 | 1.00 | 44.22 | D |
| ATOM | 12826 | CB | TYR | D | 250 | 52.291 | 18.309 | −59.932 | 1.00 | 45.51 | D |
| ATOM | 12827 | CG | TYR | D | 250 | 53.774 | 18.393 | −59.687 | 1.00 | 35.86 | D |
| ATOM | 12828 | CD1 | TYR | D | 250 | 54.565 | 17.236 | −59.623 | 1.00 | 35.86 | D |
| ATOM | 12829 | CE1 | TYR | D | 250 | 55.937 | 17.307 | −59.344 | 1.00 | 35.86 | D |
| ATOM | 12830 | CD2 | TYR | D | 250 | 54.390 | 19.627 | −59.469 | 1.00 | 35.86 | D |
| ATOM | 12831 | CE2 | TYR | D | 250 | 55.761 | 19.712 | −59.187 | 1.00 | 35.86 | D |
| ATOM | 12832 | CZ | TYR | D | 250 | 56.529 | 18.549 | −59.125 | 1.00 | 35.86 | D |
| ATOM | 12833 | OH | TYR | D | 250 | 57.875 | 18.637 | −58.831 | 1.00 | 35.86 | D |
| ATOM | 12834 | C | TYR | D | 250 | 51.912 | 16.913 | −57.944 | 1.00 | 44.22 | D |
| ATOM | 12835 | O | TYR | D | 250 | 52.764 | 16.924 | −57.056 | 1.00 | 44.22 | D |
| ATOM | 12836 | N | GLU | D | 251 | 51.289 | 15.820 | −58.365 | 1.00 | 55.38 | D |
| ATOM | 12837 | CA | GLU | D | 251 | 51.590 | 14.489 | −57.883 | 1.00 | 55.38 | D |
| ATOM | 12838 | CB | GLU | D | 251 | 50.504 | 13.986 | −56.940 | 1.00 | 109.64 | D |
| ATOM | 12839 | CG | GLU | D | 251 | 50.999 | 13.833 | −55.522 | 1.00 | 63.26 | D |
| ATOM | 12840 | CD | GLU | D | 251 | 49.999 | 13.159 | −54.604 | 1.00 | 63.26 | D |
| ATOM | 12841 | OE1 | GLU | D | 251 | 49.551 | 12.032 | −54.921 | 1.00 | 63.26 | D |
| ATOM | 12842 | OE2 | GLU | D | 251 | 49.668 | 13.752 | −53.552 | 1.00 | 63.26 | D |
| ATOM | 12843 | C | GLU | D | 251 | 51.619 | 13.658 | −59.162 | 1.00 | 55.38 | D |
| ATOM | 12844 | O | GLU | D | 251 | 50.634 | 13.023 | −59.528 | 1.00 | 55.38 | D |
| ATOM | 12845 | N | THR | D | 252 | 52.750 | 13.712 | −59.859 | 1.00 | 47.09 | D |
| ATOM | 12846 | CA | THR | D | 252 | 52.958 | 12.997 | −61.117 | 1.00 | 47.09 | D |
| ATOM | 12847 | CB | THR | D | 252 | 53.829 | 13.838 | −62.072 | 1.00 | 59.29 | D |
| ATOM | 12848 | OG1 | THR | D | 252 | 53.266 | 15.154 | −62.202 | 1.00 | 59.29 | D |
| ATOM | 12849 | CG2 | THR | D | 252 | 53.917 | 13.172 | −63.438 | 1.00 | 59.29 | D |
| ATOM | 12850 | C | THR | D | 252 | 53.689 | 11.697 | −60.807 | 1.00 | 47.09 | D |
| ATOM | 12851 | O | THR | D | 252 | 54.918 | 11.688 | −60.691 | 1.00 | 47.09 | D |
| ATOM | 12852 | N | GLY | D | 253 | 52.943 | 10.605 | −60.675 | 1.00 | 44.04 | D |
| ATOM | 12853 | CA | GLY | D | 253 | 53.575 | 9.344 | −60.334 | 1.00 | 44.04 | D |
| ATOM | 12854 | C | GLY | D | 253 | 54.254 | 9.487 | −58.974 | 1.00 | 44.04 | D |
| ATOM | 12855 | O | GLY | D | 253 | 53.644 | 9.985 | −58.014 | 1.00 | 44.04 | D |
| ATOM | 12856 | N | LEU | D | 254 | 55.512 | 9.068 | −58.878 | 1.00 | 46.01 | D |
| ATOM | 12857 | CA | LEU | D | 254 | 56.246 | 9.184 | −57.625 | 1.00 | 46.01 | D |
| ATOM | 12858 | CB | LEU | D | 254 | 57.512 | 8.330 | −57.640 | 1.00 | 40.40 | D |
| ATOM | 12859 | CG | LEU | D | 254 | 57.384 | 6.813 | −57.616 | 1.00 | 40.40 | D |
| ATOM | 12860 | CD1 | LEU | D | 254 | 58.760 | 6.223 | −57.432 | 1.00 | 40.40 | D |
| ATOM | 12861 | CD2 | LEU | D | 254 | 56.474 | 6.381 | −56.502 | 1.00 | 40.40 | D |
| ATOM | 12862 | C | LEU | D | 254 | 56.662 | 10.616 | −57.331 | 1.00 | 46.01 | D |
| ATOM | 12863 | O | LEU | D | 254 | 56.840 | 10.983 | −56.167 | 1.00 | 46.01 | D |
| ATOM | 12864 | N | LEU | D | 255 | 56.837 | 11.413 | −58.383 | 1.00 | 39.32 | D |
| ATOM | 12865 | CA | LEU | D | 255 | 57.257 | 12.803 | −58.216 | 1.00 | 39.32 | D |
| ATOM | 12866 | CB | LEU | D | 255 | 57.746 | 13.372 | −59.551 | 1.00 | 30.89 | D |
| ATOM | 12867 | CG | LEU | D | 255 | 58.815 | 12.563 | −60.299 | 1.00 | 30.89 | D |
| ATOM | 12868 | CD1 | LEU | D | 255 | 59.230 | 13.306 | −61.555 | 1.00 | 30.89 | D |
| ATOM | 12869 | CD2 | LEU | D | 255 | 60.016 | 12.327 | −59.413 | 1.00 | 30.89 | D |
| ATOM | 12870 | C | LEU | D | 255 | 56.153 | 13.694 | −57.650 | 1.00 | 39.32 | D |
| ATOM | 12871 | O | LEU | D | 255 | 54.969 | 13.450 | −57.868 | 1.00 | 39.32 | D |
| ATOM | 12872 | N | PHE | D | 256 | 56.552 | 14.728 | −56.918 | 1.00 | 39.83 | D |
| ATOM | 12873 | CA | PHE | D | 256 | 55.593 | 15.644 | −56.318 | 1.00 | 39.83 | D |
| ATOM | 12874 | CB | PHE | D | 256 | 54.907 | 14.985 | −55.115 | 1.00 | 33.29 | D |
| ATOM | 12875 | CG | PHE | D | 256 | 55.823 | 14.720 | −53.958 | 1.00 | 30.30 | D |
| ATOM | 12876 | CD1 | PHE | D | 256 | 55.900 | 15.605 | −52.896 | 1.00 | 30.30 | D |
| ATOM | 12877 | CD2 | PHE | D | 256 | 56.586 | 13.558 | −53.908 | 1.00 | 30.30 | D |
| ATOM | 12878 | CE1 | PHE | D | 256 | 56.709 | 15.334 | −51.802 | 1.00 | 30.30 | D |
| ATOM | 12879 | CE2 | PHE | D | 256 | 57.399 | 13.286 | −52.816 | 1.00 | 30.30 | D |
| ATOM | 12880 | CZ | PHE | D | 256 | 57.455 | 14.175 | −51.766 | 1.00 | 30.30 | D |
| ATOM | 12881 | C | PHE | D | 256 | 56.285 | 16.916 | −55.878 | 1.00 | 39.83 | D |
| ATOM | 12882 | O | PHE | D | 256 | 57.410 | 16.881 | −55.395 | 1.00 | 39.83 | D |
| ATOM | 12883 | N | GLY | D | 257 | 55.608 | 18.043 | −56.054 | 1.00 | 39.69 | D |
| ATOM | 12884 | CA | GLY | D | 257 | 56.185 | 19.314 | −55.665 | 1.00 | 39.69 | D |
| ATOM | 12885 | C | GLY | D | 257 | 55.191 | 20.438 | −55.828 | 1.00 | 39.69 | D |
| ATOM | 12886 | O | GLY | D | 257 | 54.056 | 20.195 | −56.217 | 1.00 | 39.69 | D |
| ATOM | 12887 | N | SER | D | 258 | 55.610 | 21.662 | −55.529 | 1.00 | 39.84 | D |
| ATOM | 12888 | CA | SER | D | 258 | 54.740 | 22.824 | −55.659 | 1.00 | 39.84 | D |
| ATOM | 12889 | CB | SER | D | 258 | 54.186 | 23.233 | −54.299 | 1.00 | 28.76 | D |
| ATOM | 12890 | OG | SER | D | 258 | 55.207 | 23.767 | −53.469 | 1.00 | 38.88 | D |
| ATOM | 12891 | C | SER | D | 258 | 55.531 | 23.981 | −56.255 | 1.00 | 39.84 | D |
| ATOM | 12892 | O | SER | D | 258 | 56.755 | 23.972 | −56.248 | 1.00 | 39.84 | D |
| ATOM | 12893 | N | TYR | D | 259 | 54.838 | 24.983 | −56.768 | 1.00 | 38.52 | D |
| ATOM | 12894 | CA | TYR | D | 259 | 55.526 | 26.108 | −57.367 | 1.00 | 38.52 | D |
| ATOM | 12895 | CB | TYR | D | 259 | 55.774 | 25.834 | −58.854 | 1.00 | 44.69 | D |
| ATOM | 12896 | CG | TYR | D | 259 | 54.517 | 25.472 | −59.587 | 1.00 | 40.44 | D |
| ATOM | 12897 | CD1 | TYR | D | 259 | 53.625 | 26.457 | −60.029 | 1.00 | 40.44 | D |
| ATOM | 12898 | CE1 | TYR | D | 259 | 52.410 | 26.114 | −60.606 | 1.00 | 40.44 | D |
| ATOM | 12899 | CD2 | TYR | D | 259 | 54.159 | 24.138 | −59.756 | 1.00 | 40.44 | D |
| ATOM | 12900 | CE2 | TYR | D | 259 | 52.951 | 23.788 | −60.332 | 1.00 | 40.44 | D |
| ATOM | 12901 | CZ | TYR | D | 259 | 52.081 | 24.780 | −60.748 | 1.00 | 40.44 | D |
| ATOM | 12902 | OH | TYR | D | 259 | 50.865 | 24.432 | −61.271 | 1.00 | 40.44 | D |
| ATOM | 12903 | C | TYR | D | 259 | 54.747 | 27.402 | −57.209 | 1.00 | 38.52 | D |
| ATOM | 12904 | O | TYR | D | 259 | 53.554 | 27.397 | −56.912 | 1.00 | 38.52 | D |
| ATOM | 12905 | N | GLY | D | 260 | 55.447 | 28.510 | −57.418 | 1.00 | 53.63 | D |
| ATOM | 12906 | CA | GLY | D | 260 | 54.849 | 29.820 | −57.306 | 1.00 | 53.63 | D |
| ATOM | 12907 | C | GLY | D | 260 | 55.822 | 30.798 | −57.918 | 1.00 | 53.63 | D |
| ATOM | 12908 | O | GLY | D | 260 | 56.855 | 31.114 | −57.335 | 1.00 | 53.63 | D |
| ATOM | 12909 | N | TYR | D | 261 | 55.510 | 31.265 | −59.115 | 1.00 | 46.47 | D |
| ATOM | 12910 | CA | TYR | D | 261 | 56.380 | 32.213 | −59.783 | 1.00 | 46.47 | D |
| ATOM | 12911 | CB | TYR | D | 261 | 57.315 | 31.480 | −60.732 | 1.00 | 53.54 | D |
| ATOM | 12912 | CG | TYR | D | 261 | 56.612 | 30.717 | −61.829 | 1.00 | 37.33 | D |
| ATOM | 12913 | CD1 | TYR | D | 261 | 56.285 | 31.331 | −63.036 | 1.00 | 37.33 | D |
| ATOM | 12914 | CE1 | TYR | D | 261 | 55.696 | 30.606 | −64.074 | 1.00 | 37.33 | D |
| ATOM | 12915 | CD2 | TYR | D | 261 | 56.322 | 29.369 | −61.682 | 1.00 | 37.33 | D |
| ATOM | 12916 | CE2 | TYR | D | 261 | 55.734 | 28.641 | −62.705 | 1.00 | 37.33 | D |
| ATOM | 12917 | CZ | TYR | D | 261 | 55.426 | 29.258 | −63.899 | 1.00 | 37.33 | D |
| ATOM | 12918 | OH | TYR | D | 261 | 54.872 | 28.522 | −64.920 | 1.00 | 37.33 | D |
| ATOM | 12919 | C | TYR | D | 261 | 55.586 | 33.262 | −60.539 | 1.00 | 46.47 | D |
| ATOM | 12920 | O | TYR | D | 261 | 54.397 | 33.097 | −60.795 | 1.00 | 46.47 | D |
| ATOM | 12921 | N | VAL | D | 262 | 56.266 | 34.344 | −60.890 | 1.00 | 45.58 | D |
| ATOM | 12922 | CA | VAL | D | 262 | 55.658 | 35.452 | −61.607 | 1.00 | 45.58 | D |
| ATOM | 12923 | CB | VAL | D | 262 | 55.899 | 36.781 | −60.864 | 1.00 | 40.73 | D |
| ATOM | 12924 | CG1 | VAL | D | 262 | 55.496 | 37.943 | −61.742 | 1.00 | 32.22 | D |
| ATOM | 12925 | CG2 | VAL | D | 262 | 55.112 | 36.807 | −59.571 | 1.00 | 32.22 | D |
| ATOM | 12926 | C | VAL | D | 262 | 56.270 | 35.566 | −62.986 | 1.00 | 45.58 | D |
| ATOM | 12927 | O | VAL | D | 262 | 57.477 | 35.681 | −63.111 | 1.00 | 45.58 | D |
| ATOM | 12928 | N | GLU | D | 263 | 55.447 | 35.508 | −64.023 | 1.00 | 52.00 | D |
| ATOM | 12929 | CA | GLU | D | 263 | 55.961 | 35.649 | −65.374 | 1.00 | 52.00 | D |
| ATOM | 12930 | CB | GLU | D | 263 | 55.717 | 34.391 | −66.214 | 1.00 | 112.72 | D |
| ATOM | 12931 | CG | GLU | D | 263 | 54.294 | 33.919 | −66.267 | 1.00 | 99.32 | D |
| ATOM | 12932 | CD | GLU | D | 263 | 54.133 | 32.685 | −67.139 | 1.00 | 99.32 | D |
| ATOM | 12933 | OE1 | GLU | D | 263 | 55.004 | 31.785 | −67.071 | 1.00 | 99.32 | D |
| ATOM | 12934 | OE2 | GLU | D | 263 | 53.129 | 32.610 | −67.884 | 1.00 | 99.32 | D |
| ATOM | 12935 | C | GLU | D | 263 | 55.342 | 36.870 | −66.037 | 1.00 | 52.00 | D |
| ATOM | 12936 | O | GLU | D | 263 | 54.317 | 37.404 | −65.602 | 1.00 | 52.00 | D |
| ATOM | 12937 | N | THR | D | 264 | 56.002 | 37.314 | −67.092 | 1.00 | 56.74 | D |
| ATOM | 12938 | CA | THR | D | 264 | 55.599 | 38.487 | −67.846 | 1.00 | 56.74 | D |
| ATOM | 12939 | CB | THR | D | 264 | 56.749 | 39.514 | −67.791 | 1.00 | 76.16 | D |
| ATOM | 12940 | OG1 | THR | D | 264 | 56.434 | 40.509 | −66.814 | 1.00 | 55.09 | D |
| ATOM | 12941 | CG2 | THR | D | 264 | 57.010 | 40.149 | −69.127 | 1.00 | 55.09 | D |
| ATOM | 12942 | C | THR | D | 264 | 55.297 | 38.075 | −69.276 | 1.00 | 56.74 | D |
| ATOM | 12943 | O | THR | D | 264 | 55.599 | 36.954 | −69.680 | 1.00 | 56.74 | D |
| ATOM | 12944 | N | ALA | D | 265 | 54.683 | 38.962 | −70.043 | 1.00 | 67.41 | D |
| ATOM | 12945 | CA | ALA | D | 265 | 54.390 | 38.631 | −71.427 | 1.00 | 67.41 | D |
| ATOM | 12946 | CB | ALA | D | 265 | 53.643 | 39.768 | −72.090 | 1.00 | 67.70 | D |
| ATOM | 12947 | C | ALA | D | 265 | 55.711 | 38.376 | −72.143 | 1.00 | 67.41 | D |
| ATOM | 12948 | O | ALA | D | 265 | 55.742 | 37.702 | −73.171 | 1.00 | 67.41 | D |
| ATOM | 12949 | N | ASN | D | 266 | 56.796 | 38.912 | −71.582 | 1.00 | 65.38 | D |
| ATOM | 12950 | CA | ASN | D | 266 | 58.135 | 38.758 | −72.146 | 1.00 | 65.38 | D |
| ATOM | 12951 | CB | ASN | D | 266 | 59.016 | 39.946 | −71.759 | 1.00 | 128.47 | D |
| ATOM | 12952 | CG | ASN | D | 266 | 58.404 | 41.273 | −72.145 | 1.00 | 70.32 | D |
| ATOM | 12953 | OD1 | ASN | D | 266 | 57.999 | 41.475 | −73.290 | 1.00 | 70.32 | D |
| ATOM | 12954 | ND2 | ASN | D | 266 | 58.339 | 42.190 | −71.191 | 1.00 | 70.32 | D |
| ATOM | 12955 | C | ASN | D | 266 | 58.818 | 37.466 | −71.703 | 1.00 | 65.38 | D |
| ATOM | 12956 | O | ASN | D | 266 | 59.833 | 37.066 | −72.279 | 1.00 | 65.38 | D |
| ATOM | 12957 | N | GLY | D | 267 | 58.264 | 36.817 | −70.682 | 1.00 | 73.10 | D |
| ATOM | 12958 | CA | GLY | D | 267 | 58.837 | 35.576 | −70.187 | 1.00 | 73.10 | D |
| ATOM | 12959 | C | GLY | D | 267 | 58.973 | 35.514 | −68.673 | 1.00 | 73.10 | D |
| ATOM | 12960 | O | GLY | D | 267 | 58.727 | 36.503 | −67.975 | 1.00 | 73.10 | D |
| ATOM | 12961 | N | PRO | D | 268 | 59.387 | 34.357 | −68.133 | 1.00 | 56.41 | D |
| ATOM | 12962 | CD | PRO | D | 268 | 59.976 | 33.229 | −68.870 | 1.00 | 53.29 | D |
| ATOM | 12963 | CA | PRO | D | 268 | 59.553 | 34.174 | −66.687 | 1.00 | 56.41 | D |
| ATOM | 12964 | CB | PRO | D | 268 | 60.277 | 32.832 | −66.587 | 1.00 | 69.18 | D |
| ATOM | 12965 | CG | PRO | D | 268 | 61.004 | 32.730 | −67.887 | 1.00 | 53.29 | D |
| ATOM | 12966 | C | PRO | D | 268 | 60.319 | 35.309 | −66.020 | 1.00 | 56.41 | D |
| ATOM | 12967 | O | PRO | D | 268 | 61.335 | 35.772 | −66.524 | 1.00 | 56.41 | D |
| ATOM | 12968 | N | LEU | D | 269 | 59.826 | 35.749 | −64.873 | 1.00 | 62.06 | D |
| ATOM | 12969 | CA | LEU | D | 269 | 60.444 | 36.847 | −64.151 | 1.00 | 62.06 | D |
| ATOM | 12970 | CB | LEU | D | 269 | 59.383 | 37.910 | −63.874 | 1.00 | 87.93 | D |
| ATOM | 12971 | CG | LEU | D | 269 | 59.861 | 39.298 | −63.471 | 1.00 | 53.27 | D |
| ATOM | 12972 | CD1 | LEU | D | 269 | 60.836 | 39.804 | −64.502 | 1.00 | 53.27 | D |
| ATOM | 12973 | CD2 | LEU | D | 269 | 58.678 | 40.241 | −63.357 | 1.00 | 53.27 | D |
| ATOM | 12974 | C | LEU | D | 269 | 61.100 | 36.391 | −62.843 | 1.00 | 62.06 | D |
| ATOM | 12975 | O | LEU | D | 269 | 62.328 | 36.321 | −62.745 | 1.00 | 62.06 | D |
| ATOM | 12976 | N | ALA | D | 270 | 60.287 | 36.077 | −61.837 | 1.00 | 54.73 | D |
| ATOM | 12977 | CA | ALA | D | 270 | 60.806 | 35.635 | −60.540 | 1.00 | 54.73 | D |
| ATOM | 12978 | CB | ALA | D | 270 | 60.819 | 36.796 | −59.566 | 1.00 | 56.20 | D |
| ATOM | 12979 | C | ALA | D | 270 | 60.047 | 34.448 | −59.927 | 1.00 | 54.73 | D |
| ATOM | 12980 | O | ALA | D | 270 | 58.830 | 34.314 | −60.064 | 1.00 | 54.73 | D |
| ATOM | 12981 | N | ARG | D | 271 | 60.798 | 33.607 | −59.229 | 1.00 | 60.30 | D |
| ATOM | 12982 | CA | ARG | D | 271 | 60.291 | 32.400 | −58.590 | 1.00 | 60.30 | D |
| ATOM | 12983 | CB | ARG | D | 271 | 61.160 | 31.227 | −59.043 | 1.00 | 44.16 | D |
| ATOM | 12984 | CG | ARG | D | 271 | 60.983 | 29.966 | −58.294 | 1.00 | 37.28 | D |
| ATOM | 12985 | CD | ARG | D | 271 | 61.980 | 28.921 | −58.793 | 1.00 | 37.28 | D |
| ATOM | 12986 | NE | ARG | D | 271 | 61.691 | 28.448 | −60.150 | 1.00 | 37.28 | D |
| ATOM | 12987 | CZ | ARG | D | 271 | 62.389 | 28.772 | −61.232 | 1.00 | 37.28 | D |
| ATOM | 12988 | NH1 | ARG | D | 271 | 63.429 | 29.585 | −61.136 | 1.00 | 37.28 | D |
| ATOM | 12989 | NH2 | ARG | D | 271 | 62.059 | 28.262 | −62.405 | 1.00 | 37.28 | D |
| ATOM | 12990 | C | ARG | D | 271 | 60.343 | 32.569 | −57.078 | 1.00 | 60.30 | D |
| ATOM | 12991 | O | ARG | D | 271 | 61.329 | 33.053 | −56.533 | 1.00 | 60.30 | D |
| ATOM | 12992 | N | TYR | D | 272 | 59.279 | 32.179 | −56.394 | 1.00 | 42.35 | D |
| ATOM | 12993 | CA | TYR | D | 272 | 59.238 | 32.325 | −54.944 | 1.00 | 42.35 | D |
| ATOM | 12994 | CB | TYR | D | 272 | 57.816 | 32.607 | −54.477 | 1.00 | 44.17 | D |
| ATOM | 12995 | CG | TYR | D | 272 | 57.713 | 32.795 | −52.986 | 1.00 | 41.64 | D |
| ATOM | 12996 | CD1 | TYR | D | 272 | 57.985 | 34.025 | −52.410 | 1.00 | 41.64 | D |
| ATOM | 12997 | CE1 | TYR | D | 272 | 57.906 | 34.211 | −51.023 | 1.00 | 41.64 | D |
| ATOM | 12998 | CD2 | TYR | D | 272 | 57.358 | 31.737 | −52.144 | 1.00 | 41.64 | D |
| ATOM | 12999 | CE2 | TYR | D | 272 | 57.278 | 31.910 | −50.761 | 1.00 | 41.64 | D |
| ATOM | 13000 | CZ | TYR | D | 272 | 57.554 | 33.152 | −50.210 | 1.00 | 41.64 | D |
| ATOM | 13001 | OH | TYR | D | 272 | 57.482 | 33.344 | −48.848 | 1.00 | 41.64 | D |
| ATOM | 13002 | C | TYR | D | 272 | 59.744 | 31.078 | −54.233 | 1.00 | 42.35 | D |
| ATOM | 13003 | O | TYR | D | 272 | 59.206 | 29.987 | −54.422 | 1.00 | 42.35 | D |
| ATOM | 13004 | N | HIS | D | 273 | 60.764 | 31.239 | −53.397 | 1.00 | 62.73 | D |
| ATOM | 13005 | CA | HIS | D | 273 | 61.319 | 30.108 | −52.664 | 1.00 | 62.73 | D |
| ATOM | 13006 | CB | HIS | D | 273 | 62.844 | 30.148 | −52.690 | 1.00 | 122.43 | D |
| ATOM | 13007 | CG | HIS | D | 273 | 63.419 | 29.753 | −54.010 | 1.00 | 103.16 | D |
| ATOM | 13008 | CD2 | HIS | D | 273 | 64.190 | 30.433 | −54.890 | 1.00 | 103.16 | D |
| ATOM | 13009 | ND1 | HIS | D | 273 | 63.179 | 28.522 | −54.580 | 1.00 | 103.16 | D |
| ATOM | 13010 | CE1 | HIS | D | 273 | 63.777 | 28.460 | −55.757 | 1.00 | 103.16 | D |
| ATOM | 13011 | NE2 | HIS | D | 273 | 64.398 | 29.607 | −55.969 | 1.00 | 103.16 | D |
| ATOM | 13012 | C | HIS | D | 273 | 60.825 | 30.026 | −51.236 | 1.00 | 62.73 | D |
| ATOM | 13013 | O | HIS | D | 273 | 61.013 | 30.936 | −50.432 | 1.00 | 62.73 | D |
| ATOM | 13014 | N | THR | D | 274 | 60.173 | 28.912 | −50.944 | 1.00 | 58.88 | D |
| ATOM | 13015 | CA | THR | D | 274 | 59.625 | 28.633 | −49.629 | 1.00 | 58.88 | D |
| ATOM | 13016 | CB | THR | D | 274 | 58.975 | 27.240 | −49.625 | 1.00 | 67.78 | D |
| ATOM | 13017 | OG1 | THR | D | 274 | 57.567 | 27.386 | −49.830 | 1.00 | 60.31 | D |
| ATOM | 13018 | CG2 | THR | D | 274 | 59.247 | 26.504 | −48.325 | 1.00 | 60.31 | D |
| ATOM | 13019 | C | THR | D | 274 | 60.682 | 28.714 | −48.536 | 1.00 | 58.88 | D |
| ATOM | 13020 | O | THR | D | 274 | 60.404 | 29.176 | −47.434 | 1.00 | 58.88 | D |
| ATOM | 13021 | N | GLY | D | 275 | 61.893 | 28.260 | −48.849 | 1.00 | 62.83 | D |
| ATOM | 13022 | CA | GLY | D | 275 | 62.977 | 28.292 | −47.881 | 1.00 | 62.83 | D |
| ATOM | 13023 | C | GLY | D | 275 | 63.431 | 29.695 | −47.513 | 1.00 | 62.83 | D |
| ATOM | 13024 | O | GLY | D | 275 | 63.301 | 30.110 | −46.366 | 1.00 | 62.83 | D |
| ATOM | 13025 | N | ASP | D | 276 | 63.952 | 30.428 | −48.490 | 1.00 | 62.55 | D |
| ATOM | 13026 | CA | ASP | D | 276 | 64.431 | 31.789 | −48.276 | 1.00 | 62.55 | D |
| ATOM | 13027 | CB | ASP | D | 276 | 65.203 | 32.251 | −49.504 | 1.00 | 155.37 | D |
| ATOM | 13028 | CG | ASP | D | 276 | 66.218 | 31.236 | −49.957 | 1.00 | 134.37 | D |
| ATOM | 13029 | OD1 | ASP | D | 276 | 67.209 | 31.026 | −49.227 | 1.00 | 134.37 | D |
| ATOM | 13030 | OD2 | ASP | D | 276 | 66.017 | 30.640 | −51.037 | 1.00 | 134.37 | D |
| ATOM | 13031 | C | ASP | D | 276 | 63.332 | 32.813 | −47.976 | 1.00 | 62.55 | D |
| ATOM | 13032 | O | ASP | D | 276 | 63.621 | 33.904 | −47.478 | 1.00 | 62.55 | D |
| ATOM | 13033 | N | LYS | D | 277 | 62.082 | 32.469 | −48.287 | 1.00 | 65.82 | D |
| ATOM | 13034 | CA | LYS | D | 277 | 60.954 | 33.370 | −48.066 | 1.00 | 65.82 | D |
| ATOM | 13035 | CB | LYS | D | 277 | 60.783 | 33.633 | −46.572 | 1.00 | 83.96 | D |
| ATOM | 13036 | CG | LYS | D | 277 | 60.346 | 32.407 | −45.789 | 1.00 | 69.17 | D |
| ATOM | 13037 | CD | LYS | D | 277 | 60.503 | 32.635 | −44.296 | 1.00 | 69.17 | D |
| ATOM | 13038 | CE | LYS | D | 277 | 60.010 | 31.454 | −43.467 | 1.00 | 69.17 | D |
| ATOM | 13039 | NZ | LYS | D | 277 | 58.519 | 31.393 | −43.395 | 1.00 | 69.17 | D |
| ATOM | 13040 | C | LYS | D | 277 | 61.151 | 34.686 | −48.826 | 1.00 | 65.82 | D |
| ATOM | 13041 | O | LYS | D | 277 | 61.016 | 35.776 | −48.270 | 1.00 | 65.82 | D |
| ATOM | 13042 | N | ARG | D | 278 | 61.470 | 34.563 | −50.109 | 1.00 | 46.40 | D |
| ATOM | 13043 | CA | ARG | D | 278 | 61.693 | 35.712 | −50.975 | 1.00 | 46.40 | D |
| ATOM | 13044 | CB | ARG | D | 278 | 63.045 | 36.340 | −50.652 | 1.00 | 105.48 | D |
| ATOM | 13045 | CG | ARG | D | 278 | 64.204 | 35.477 | −51.075 | 1.00 | 90.11 | D |
| ATOM | 13046 | CD | ARG | D | 278 | 65.510 | 35.976 | −50.513 | 1.00 | 90.11 | D |
| ATOM | 13047 | NE | ARG | D | 278 | 66.642 | 35.202 | −51.025 | 1.00 | 90.11 | D |
| ATOM | 13048 | CZ | ARG | D | 278 | 67.866 | 35.225 | −50.502 | 1.00 | 90.11 | D |
| ATOM | 13049 | NH1 | ARG | D | 278 | 68.121 | 35.985 | −49.442 | 1.00 | 90.11 | D |
| ATOM | 13050 | NH2 | ARG | D | 278 | 68.834 | 34.493 | −51.042 | 1.00 | 90.11 | D |
| ATOM | 13051 | C | ARG | D | 278 | 61.683 | 35.233 | −52.433 | 1.00 | 46.40 | D |
| ATOM | 13052 | O | ARG | D | 278 | 61.691 | 34.028 | −52.706 | 1.00 | 46.40 | D |
| ATOM | 13053 | N | PHE | D | 279 | 61.657 | 36.168 | −53.375 | 1.00 | 48.84 | D |
| ATOM | 13054 | CA | PHE | D | 279 | 61.672 | 35.802 | −54.790 | 1.00 | 48.84 | D |
| ATOM | 13055 | CB | PHE | D | 279 | 60.932 | 36.833 | −55.642 | 1.00 | 60.99 | D |
| ATOM | 13056 | CG | PHE | D | 279 | 59.449 | 36.693 | −55.610 | 1.00 | 44.76 | D |
| ATOM | 13057 | CD1 | PHE | D | 279 | 58.685 | 37.391 | −54.676 | 1.00 | 44.76 | D |
| ATOM | 13058 | CD2 | PHE | D | 279 | 58.806 | 35.864 | −56.519 | 1.00 | 44.76 | D |
| ATOM | 13059 | CE1 | PHE | D | 279 | 57.296 | 37.265 | −54.654 | 1.00 | 44.76 | D |
| ATOM | 13060 | CE2 | PHE | D | 279 | 57.424 | 35.729 | −56.507 | 1.00 | 44.76 | D |
| ATOM | 13061 | CZ | PHE | D | 279 | 56.666 | 36.432 | −55.573 | 1.00 | 44.76 | D |
| ATOM | 13062 | C | PHE | D | 279 | 63.105 | 35.705 | −55.293 | 1.00 | 48.84 | D |
| ATOM | 13063 | O | PHE | D | 279 | 64.006 | 36.351 | −54.754 | 1.00 | 48.84 | D |
| ATOM | 13064 | N | THR | D | 280 | 63.303 | 34.902 | −56.334 | 1.00 | 49.98 | D |
| ATOM | 13065 | CA | THR | D | 280 | 64.615 | 34.718 | −56.923 | 1.00 | 49.98 | D |
| ATOM | 13066 | CB | THR | D | 280 | 65.215 | 33.372 | −56.543 | 1.00 | 91.39 | D |
| ATOM | 13067 | OG1 | THR | D | 280 | 65.150 | 33.212 | −55.122 | 1.00 | 71.91 | D |
| ATOM | 13068 | CG2 | THR | D | 280 | 66.666 | 33.298 | −57.004 | 1.00 | 71.91 | D |
| ATOM | 13069 | C | THR | D | 280 | 64.517 | 34.760 | −58.431 | 1.00 | 49.98 | D |
| ATOM | 13070 | O | THR | D | 280 | 63.615 | 34.177 | −59.007 | 1.00 | 49.98 | D |
| ATOM | 13071 | N | LYS | D | 281 | 65.458 | 35.445 | −59.067 | 1.00 | 70.17 | D |
| ATOM | 13072 | CA | LYS | D | 281 | 65.488 | 35.562 | −60.519 | 1.00 | 70.17 | D |
| ATOM | 13073 | CB | LYS | D | 281 | 66.806 | 36.214 | −60.944 | 1.00 | 143.55 | D |
| ATOM | 13074 | CG | LYS | D | 281 | 67.992 | 35.793 | −60.081 | 1.00 | 119.49 | D |
| ATOM | 13075 | CD | LYS | D | 281 | 69.185 | 35.346 | −60.916 | 1.00 | 119.49 | D |
| ATOM | 13076 | CE | LYS | D | 281 | 69.790 | 36.481 | −61.734 | 1.00 | 119.49 | D |
| ATOM | 13077 | NZ | LYS | D | 281 | 71.001 | 36.044 | −62.501 | 1.00 | 119.49 | D |
| ATOM | 13078 | C | LYS | D | 281 | 65.336 | 34.210 | −61.213 | 1.00 | 70.17 | D |
| ATOM | 13079 | O | LYS | D | 281 | 65.865 | 33.203 | −60.751 | 1.00 | 70.17 | D |
| ATOM | 13080 | N | MET | D | 282 | 64.596 | 34.191 | −62.316 | 1.00 | 51.84 | D |
| ATOM | 13081 | CA | MET | D | 282 | 64.414 | 32.962 | −63.081 | 1.00 | 51.84 | D |
| ATOM | 13082 | CB | MET | D | 282 | 62.950 | 32.764 | −63.468 | 1.00 | 66.83 | D |
| ATOM | 13083 | CG | MET | D | 282 | 62.031 | 32.400 | −62.326 | 1.00 | 66.83 | D |
| ATOM | 13084 | SD | MET | D | 282 | 60.468 | 31.741 | −62.943 | 1.00 | 66.83 | D |
| ATOM | 13085 | CE | MET | D | 282 | 59.509 | 33.228 | −63.105 | 1.00 | 66.83 | D |
| ATOM | 13086 | C | MET | D | 282 | 65.242 | 33.060 | −64.353 | 1.00 | 51.84 | D |
| ATOM | 13087 | O | MET | D | 282 | 65.382 | 32.093 | −65.094 | 1.00 | 51.84 | D |
| ATOM | 13088 | N | THR | D | 283 | 65.780 | 34.251 | −64.598 | 1.00 | 91.08 | D |
| ATOM | 13089 | CA | THR | D | 283 | 66.593 | 34.524 | −65.776 | 1.00 | 91.08 | D |
| ATOM | 13090 | CB | THR | D | 283 | 65.801 | 35.359 | −66.793 | 1.00 | 73.84 | D |
| ATOM | 13091 | OG1 | THR | D | 283 | 64.688 | 34.596 | −67.262 | 1.00 | 73.84 | D |
| ATOM | 13092 | CG2 | THR | D | 283 | 66.668 | 35.741 | −67.970 | 1.00 | 73.84 | D |
| ATOM | 13093 | C | THR | D | 283 | 67.820 | 35.317 | −65.351 | 1.00 | 91.08 | D |
| ATOM | 13094 | O | THR | D | 283 | 67.818 | 35.942 | −64.290 | 1.00 | 91.08 | D |
| ATOM | 13095 | N | HIS | D | 284 | 68.865 | 35.281 | −66.174 | 1.00 | 95.17 | D |
| ATOM | 13096 | CA | HIS | D | 284 | 70.093 | 36.024 | −65.899 | 1.00 | 95.17 | D |
| ATOM | 13097 | CB | HIS | D | 284 | 71.093 | 35.825 | −67.043 | 1.00 | 137.09 | D |
| ATOM | 13098 | CG | HIS | D | 284 | 70.552 | 36.205 | −68.390 | 1.00 | 137.09 | D |
| ATOM | 13099 | CD2 | HIS | D | 284 | 70.915 | 37.182 | −69.255 | 1.00 | 137.09 | D |
| ATOM | 13100 | ND1 | HIS | D | 284 | 69.485 | 35.555 | −68.975 | 1.00 | 137.09 | D |
| ATOM | 13101 | CE1 | HIS | D | 284 | 69.215 | 36.115 | −70.141 | 1.00 | 137.09 | D |
| ATOM | 13102 | NE2 | HIS | D | 284 | 70.068 | 37.105 | −70.335 | 1.00 | 137.09 | D |
| ATOM | 13103 | C | HIS | D | 284 | 69.743 | 37.509 | −65.784 | 1.00 | 95.17 | D |
| ATOM | 13104 | O | HIS | D | 284 | 70.319 | 38.239 | −64.973 | 1.00 | 95.17 | D |
| ATOM | 13105 | N | LYS | D | 285 | 68.782 | 37.929 | −66.607 | 1.00 | 81.36 | D |
| ATOM | 13106 | CA | LYS | D | 285 | 68.300 | 39.304 | −66.674 | 1.00 | 81.36 | D |
| ATOM | 13107 | CB | LYS | D | 285 | 67.075 | 39.368 | −67.587 | 1.00 | 98.23 | D |
| ATOM | 13108 | CG | LYS | D | 285 | 67.149 | 40.434 | −68.663 | 1.00 | 98.23 | D |
| ATOM | 13109 | CD | LYS | D | 285 | 68.264 | 40.139 | −69.661 | 1.00 | 98.23 | D |
| ATOM | 13110 | CE | LYS | D | 285 | 68.322 | 41.196 | −70.759 | 1.00 | 98.23 | D |
| ATOM | 13111 | NZ | LYS | D | 285 | 69.396 | 40.917 | −71.749 | 1.00 | 98.23 | D |
| ATOM | 13112 | C | LYS | D | 285 | 67.944 | 39.925 | −65.322 | 1.00 | 81.36 | D |
| ATOM | 13113 | O | LYS | D | 285 | 68.082 | 41.133 | −65.139 | 1.00 | 81.36 | D |
| ATOM | 13114 | N | GLY | D | 286 | 67.486 | 39.103 | −64.381 | 1.00 | 59.05 | D |
| ATOM | 13115 | CA | GLY | D | 286 | 67.104 | 39.609 | −63.072 | 1.00 | 59.05 | D |
| ATOM | 13116 | C | GLY | D | 286 | 65.666 | 40.094 | −63.109 | 1.00 | 59.05 | D |
| ATOM | 13117 | O | GLY | D | 286 | 64.997 | 39.954 | −64.133 | 1.00 | 59.05 | D |
| ATOM | 13118 | N | PHE | D | 287 | 65.178 | 40.656 | −62.006 | 1.00 | 61.03 | D |
| ATOM | 13119 | CA | PHE | D | 287 | 63.805 | 41.160 | −61.959 | 1.00 | 61.03 | D |
| ATOM | 13120 | CB | PHE | D | 287 | 62.864 | 40.097 | −61.389 | 1.00 | 47.22 | D |
| ATOM | 13121 | CG | PHE | D | 287 | 63.084 | 39.808 | −59.929 | 1.00 | 47.22 | D |
| ATOM | 13122 | CD1 | PHE | D | 287 | 62.460 | 40.574 | −58.952 | 1.00 | 47.22 | D |
| ATOM | 13123 | CD2 | PHE | D | 287 | 63.905 | 38.756 | −59.528 | 1.00 | 47.22 | D |
| ATOM | 13124 | CE1 | PHE | D | 287 | 62.648 | 40.297 | −57.595 | 1.00 | 47.22 | D |
| ATOM | 13125 | CE2 | PHE | D | 287 | 64.101 | 38.467 | −58.165 | 1.00 | 47.22 | D |
| ATOM | 13126 | CZ | PHE | D | 287 | 63.473 | 39.237 | −57.202 | 1.00 | 47.22 | D |
| ATOM | 13127 | C | PHE | D | 287 | 63.721 | 42.424 | −61.117 | 1.00 | 61.03 | D |
| ATOM | 13128 | O | PHE | D | 287 | 64.380 | 42.543 | −60.081 | 1.00 | 61.03 | D |
| ATOM | 13129 | N | PRO | D | 288 | 62.894 | 43.388 | −61.546 | 1.00 | 67.74 | D |
| ATOM | 13130 | CD | PRO | D | 288 | 61.994 | 43.315 | −62.711 | 1.00 | 61.29 | D |
| ATOM | 13131 | CA | PRO | D | 288 | 62.722 | 44.656 | −60.831 | 1.00 | 67.74 | D |
| ATOM | 13132 | CB | PRO | D | 288 | 61.837 | 45.462 | −61.775 | 1.00 | 61.29 | D |
| ATOM | 13133 | CG | PRO | D | 288 | 60.986 | 44.404 | −62.408 | 1.00 | 61.29 | D |
| ATOM | 13134 | C | PRO | D | 288 | 62.097 | 44.480 | −59.458 | 1.00 | 67.74 | D |
| ATOM | 13135 | O | PRO | D | 288 | 61.013 | 43.906 | −59.332 | 1.00 | 67.74 | D |
| ATOM | 13136 | N | LYS | D | 289 | 62.784 | 44.968 | −58.430 | 1.00 | 59.47 | D |
| ATOM | 13137 | CA | LYS | D | 289 | 62.268 | 44.863 | −57.074 | 1.00 | 59.47 | D |
| ATOM | 13138 | CB | LYS | D | 289 | 63.414 | 44.914 | −56.061 | 1.00 | 125.23 | D |
| ATOM | 13139 | CG | LYS | D | 289 | 64.179 | 43.598 | −55.979 | 1.00 | 125.23 | D |
| ATOM | 13140 | CD | LYS | D | 289 | 65.324 | 43.667 | −54.991 | 1.00 | 125.23 | D |
| ATOM | 13141 | CE | LYS | D | 289 | 66.445 | 44.562 | −55.511 | 1.00 | 125.23 | D |
| ATOM | 13142 | NZ | LYS | D | 289 | 67.607 | 44.618 | −54.578 | 1.00 | 125.23 | D |
| ATOM | 13143 | C | LYS | D | 289 | 61.216 | 45.941 | −56.794 | 1.00 | 59.47 | D |
| ATOM | 13144 | O | LYS | D | 289 | 61.474 | 46.951 | −56.141 | 1.00 | 59.47 | D |
| ATOM | 13145 | N | VAL | D | 290 | 60.012 | 45.687 | −57.300 | 1.00 | 71.87 | D |
| ATOM | 13146 | CA | VAL | D | 290 | 58.883 | 46.589 | −57.145 | 1.00 | 71.87 | D |
| ATOM | 13147 | CB | VAL | D | 290 | 58.647 | 47.380 | −58.444 | 1.00 | 63.85 | D |
| ATOM | 13148 | CG1 | VAL | D | 290 | 59.934 | 48.042 | −58.885 | 1.00 | 63.85 | D |
| ATOM | 13149 | CG2 | VAL | D | 290 | 58.132 | 46.446 | −59.537 | 1.00 | 63.85 | D |
| ATOM | 13150 | C | VAL | D | 290 | 57.620 | 45.788 | −56.836 | 1.00 | 71.87 | D |
| ATOM | 13151 | O | VAL | D | 290 | 57.600 | 44.569 | −56.967 | 1.00 | 71.87 | D |
| ATOM | 13152 | N | GLY | D | 291 | 56.571 | 46.490 | −56.430 | 1.00 | 57.44 | D |
| ATOM | 13153 | CA | GLY | D | 291 | 55.303 | 45.846 | −56.134 | 1.00 | 57.44 | D |
| ATOM | 13154 | C | GLY | D | 291 | 55.372 | 44.642 | −55.222 | 1.00 | 57.44 | D |
| ATOM | 13155 | O | GLY | D | 291 | 56.021 | 44.676 | −54.180 | 1.00 | 57.44 | D |
| ATOM | 13156 | N | MET | D | 292 | 54.688 | 43.577 | −55.623 | 1.00 | 64.59 | D |
| ATOM | 13157 | CA | MET | D | 292 | 54.648 | 42.335 | −54.860 | 1.00 | 64.59 | D |
| ATOM | 13158 | CB | MET | D | 292 | 53.594 | 41.410 | −55.451 | 1.00 | 76.71 | D |
| ATOM | 13159 | CG | MET | D | 292 | 54.003 | 40.900 | −56.814 | 1.00 | 76.71 | D |
| ATOM | 13160 | SD | MET | D | 292 | 53.097 | 39.456 | −57.324 | 1.00 | 76.71 | D |
| ATOM | 13161 | CE | MET | D | 292 | 52.225 | 40.098 | −58.733 | 1.00 | 76.71 | D |
| ATOM | 13162 | C | MET | D | 292 | 55.989 | 41.590 | −54.880 | 1.00 | 64.59 | D |
| ATOM | 13163 | O | MET | D | 292 | 56.121 | 40.530 | −54.279 | 1.00 | 64.59 | D |
| ATOM | 13164 | N | LEU | D | 293 | 56.980 | 42.136 | −55.571 | 1.00 | 56.28 | D |
| ATOM | 13165 | CA | LEU | D | 293 | 58.265 | 41.472 | −55.673 | 1.00 | 56.28 | D |
| ATOM | 13166 | CB | LEU | D | 293 | 58.817 | 41.642 | −57.088 | 1.00 | 52.05 | D |
| ATOM | 13167 | CG | LEU | D | 293 | 57.996 | 40.964 | −58.185 | 1.00 | 52.05 | D |
| ATOM | 13168 | CD1 | LEU | D | 293 | 58.552 | 41.299 | −59.563 | 1.00 | 52.05 | D |
| ATOM | 13169 | CD2 | LEU | D | 293 | 58.021 | 39.472 | −57.958 | 1.00 | 52.05 | D |
| ATOM | 13170 | C | LEU | D | 293 | 59.348 | 41.857 | −54.664 | 1.00 | 56.28 | D |
| ATOM | 13171 | O | LEU | D | 293 | 60.427 | 41.264 | −54.679 | 1.00 | 56.28 | D |
| ATOM | 13172 | N | THR | D | 294 | 59.089 | 42.833 | −53.796 | 1.00 | 66.61 | D |
| ATOM | 13173 | CA | THR | D | 294 | 60.097 | 43.222 | −52.807 | 1.00 | 66.61 | D |
| ATOM | 13174 | CB | THR | D | 294 | 60.617 | 44.645 | −53.007 | 1.00 | 86.83 | D |
| ATOM | 13175 | OG1 | THR | D | 294 | 60.762 | 44.916 | −54.401 | 1.00 | 86.83 | D |
| ATOM | 13176 | CG2 | THR | D | 294 | 61.976 | 44.796 | −52.319 | 1.00 | 86.83 | D |
| ATOM | 13177 | C | THR | D | 294 | 59.583 | 43.180 | −51.390 | 1.00 | 66.61 | D |
| ATOM | 13178 | O | THR | D | 294 | 58.402 | 43.385 | −51.139 | 1.00 | 66.61 | D |
| ATOM | 13179 | N | VAL | D | 295 | 60.491 | 42.939 | −50.458 | 1.00 | 69.77 | D |
| ATOM | 13180 | CA | VAL | D | 295 | 60.121 | 42.894 | −49.060 | 1.00 | 69.77 | D |
| ATOM | 13181 | CB | VAL | D | 295 | 61.288 | 42.374 | −48.215 | 1.00 | 59.45 | D |
| ATOM | 13182 | CG1 | VAL | D | 295 | 60.868 | 42.263 | −46.743 | 1.00 | 59.45 | D |
| ATOM | 13183 | CG2 | VAL | D | 295 | 61.744 | 41.030 | −48.761 | 1.00 | 59.45 | D |
| ATOM | 13184 | C | VAL | D | 295 | 59.770 | 44.315 | −48.636 | 1.00 | 69.77 | D |
| ATOM | 13185 | O | VAL | D | 295 | 59.078 | 44.534 | −47.636 | 1.00 | 69.77 | D |
| ATOM | 13186 | N | LYS | D | 296 | 60.245 | 45.277 | −49.421 | 1.00 | 63.98 | D |
| ATOM | 13187 | CA | LYS | D | 296 | 60.006 | 46.690 | −49.152 | 1.00 | 63.98 | D |
| ATOM | 13188 | CB | LYS | D | 296 | 60.683 | 47.552 | −50.222 | 1.00 | 96.00 | D |
| ATOM | 13189 | CG | LYS | D | 296 | 62.197 | 47.390 | −50.317 | 1.00 | 96.00 | D |
| ATOM | 13190 | CD | LYS | D | 296 | 62.921 | 48.057 | −49.155 | 1.00 | 96.00 | D |
| ATOM | 13191 | CE | LYS | D | 296 | 62.802 | 49.574 | −49.229 | 1.00 | 96.00 | D |
| ATOM | 13192 | NZ | LYS | D | 296 | 63.476 | 50.260 | −48.092 | 1.00 | 96.00 | D |
| ATOM | 13193 | C | LYS | D | 296 | 58.515 | 47.006 | −49.128 | 1.00 | 63.98 | D |
| ATOM | 13194 | O | LYS | D | 296 | 58.071 | 47.883 | −48.373 | 1.00 | 63.98 | D |
| ATOM | 13195 | N | ASN | D | 297 | 57.749 | 46.284 | −49.951 | 1.00 | 53.99 | D |
| ATOM | 13196 | CA | ASN | D | 297 | 56.309 | 46.493 | −50.053 | 1.00 | 53.99 | D |
| ATOM | 13197 | CB | ASN | D | 297 | 55.714 | 45.590 | −51.134 | 1.00 | 65.43 | D |
| ATOM | 13198 | CG | ASN | D | 297 | 54.285 | 45.968 | −51.488 | 1.00 | 65.43 | D |
| ATOM | 13199 | OD1 | ASN | D | 297 | 53.382 | 45.912 | −50.647 | 1.00 | 65.43 | D |
| ATOM | 13200 | ND2 | ASN | D | 297 | 54.073 | 46.358 | −52.741 | 1.00 | 65.43 | D |
| ATOM | 13201 | C | ASN | D | 297 | 55.547 | 46.295 | −48.743 | 1.00 | 53.99 | D |
| ATOM | 13202 | O | ASN | D | 297 | 55.765 | 45.338 | −48.001 | 1.00 | 53.99 | D |
| ATOM | 13203 | N | THR | D | 298 | 54.641 | 47.226 | −48.478 | 1.00 | 49.34 | D |
| ATOM | 13204 | CA | THR | D | 298 | 53.825 | 47.199 | −47.286 | 1.00 | 49.34 | D |
| ATOM | 13205 | CB | THR | D | 298 | 52.934 | 48.453 | −47.244 | 1.00 | 76.17 | D |
| ATOM | 13206 | OG1 | THR | D | 298 | 51.846 | 48.232 | −46.346 | 1.00 | 76.17 | D |
| ATOM | 13207 | CG2 | THR | D | 298 | 52.391 | 48.779 | −48.627 | 1.00 | 76.17 | D |
| ATOM | 13208 | C | THR | D | 298 | 52.955 | 45.937 | −47.168 | 1.00 | 49.34 | D |
| ATOM | 13209 | O | THR | D | 298 | 52.584 | 45.541 | −46.065 | 1.00 | 49.34 | D |
| ATOM | 13210 | N | LEU | D | 299 | 52.636 | 45.300 | −48.291 | 1.00 | 56.01 | D |
| ATOM | 13211 | CA | LEU | D | 299 | 51.802 | 44.104 | −48.260 | 1.00 | 56.01 | D |
| ATOM | 13212 | CB | LEU | D | 299 | 50.790 | 44.154 | −49.394 | 1.00 | 52.60 | D |
| ATOM | 13213 | CG | LEU | D | 299 | 49.759 | 45.269 | −49.269 | 1.00 | 52.60 | D |
| ATOM | 13214 | CD1 | LEU | D | 299 | 48.915 | 45.294 | −50.527 | 1.00 | 52.60 | D |
| ATOM | 13215 | CD2 | LEU | D | 299 | 48.905 | 45.048 | −48.029 | 1.00 | 52.60 | D |
| ATOM | 13216 | C | LEU | D | 299 | 52.583 | 42.798 | −48.336 | 1.00 | 56.01 | D |
| ATOM | 13217 | O | LEU | D | 299 | 51.995 | 41.715 | −48.439 | 1.00 | 56.01 | D |
| ATOM | 13218 | N | TRP | D | 300 | 53.907 | 42.899 | −48.281 | 1.00 | 54.84 | D |
| ATOM | 13219 | CA | TRP | D | 300 | 54.760 | 41.721 | −48.345 | 1.00 | 54.84 | D |
| ATOM | 13220 | CB | TRP | D | 300 | 56.196 | 42.089 | −48.009 | 1.00 | 56.92 | D |
| ATOM | 13221 | CG | TRP | D | 300 | 57.127 | 40.933 | −48.105 | 1.00 | 56.92 | D |
| ATOM | 13222 | CD2 | TRP | D | 300 | 57.403 | 40.143 | −49.272 | 1.00 | 56.92 | D |
| ATOM | 13223 | CE2 | TRP | D | 300 | 58.356 | 39.170 | −48.903 | 1.00 | 56.92 | D |
| ATOM | 13224 | CE3 | TRP | D | 300 | 56.938 | 40.166 | −50.592 | 1.00 | 56.92 | D |
| ATOM | 13225 | CD1 | TRP | D | 300 | 57.897 | 40.420 | −47.106 | 1.00 | 56.92 | D |
| ATOM | 13226 | NE1 | TRP | D | 300 | 58.641 | 39.359 | −47.577 | 1.00 | 56.92 | D |
| ATOM | 13227 | CZ2 | TRP | D | 300 | 58.853 | 38.229 | −49.804 | 1.00 | 56.92 | D |
| ATOM | 13228 | CZ3 | TRP | D | 300 | 57.434 | 39.231 | −51.490 | 1.00 | 56.92 | D |
| ATOM | 13229 | CH2 | TRP | D | 300 | 58.382 | 38.275 | −51.092 | 1.00 | 56.92 | D |
| ATOM | 13230 | C | TRP | D | 300 | 54.307 | 40.614 | −47.408 | 1.00 | 54.84 | D |
| ATOM | 13231 | O | TRP | D | 300 | 54.308 | 39.445 | −47.790 | 1.00 | 54.84 | D |
| ATOM | 13232 | N | LYS | D | 301 | 53.924 | 40.975 | −46.186 | 1.00 | 54.12 | D |
| ATOM | 13233 | CA | LYS | D | 301 | 53.478 | 39.982 | −45.216 | 1.00 | 54.12 | D |
| ATOM | 13234 | CB | LYS | D | 301 | 53.066 | 40.646 | −43.897 | 1.00 | 81.50 | D |
| ATOM | 13235 | CG | LYS | D | 301 | 54.187 | 41.442 | −43.232 | 1.00 | 81.50 | D |
| ATOM | 13236 | CD | LYS | D | 301 | 55.495 | 40.631 | −43.155 | 1.00 | 81.50 | D |
| ATOM | 13237 | CE | LYS | D | 301 | 56.714 | 41.520 | −42.887 | 1.00 | 81.50 | D |
| ATOM | 13238 | NZ | LYS | D | 301 | 57.999 | 40.832 | −43.205 | 1.00 | 81.50 | D |
| ATOM | 13239 | C | LYS | D | 301 | 52.324 | 39.158 | −45.765 | 1.00 | 54.12 | D |
| ATOM | 13240 | O | LYS | D | 301 | 52.247 | 37.954 | −45.510 | 1.00 | 54.12 | D |
| ATOM | 13241 | N | ASP | D | 302 | 51.431 | 39.791 | −46.528 | 1.00 | 55.09 | D |
| ATOM | 13242 | CA | ASP | D | 302 | 50.307 | 39.064 | −47.096 | 1.00 | 55.09 | D |
| ATOM | 13243 | CB | ASP | D | 302 | 49.242 | 40.028 | −47.609 | 1.00 | 84.07 | D |
| ATOM | 13244 | CG | ASP | D | 302 | 48.500 | 40.707 | −46.487 | 1.00 | 84.07 | D |
| ATOM | 13245 | OD1 | ASP | D | 302 | 48.035 | 39.991 | −45.578 | 1.00 | 84.07 | D |
| ATOM | 13246 | OD2 | ASP | D | 302 | 48.378 | 41.948 | −46.510 | 1.00 | 84.07 | D |
| ATOM | 13247 | C | ASP | D | 302 | 50.788 | 38.159 | −48.211 | 1.00 | 55.09 | D |
| ATOM | 13248 | O | ASP | D | 302 | 50.295 | 37.041 | −48.356 | 1.00 | 55.09 | D |
| ATOM | 13249 | N | VAL | D | 303 | 51.753 | 38.636 | −48.994 | 1.00 | 44.08 | D |
| ATOM | 13250 | CA | VAL | D | 303 | 52.296 | 37.831 | −50.076 | 1.00 | 44.08 | D |
| ATOM | 13251 | CB | VAL | D | 303 | 53.414 | 38.568 | −50.819 | 1.00 | 48.62 | D |
| ATOM | 13252 | CG1 | VAL | D | 303 | 54.049 | 37.654 | −51.856 | 1.00 | 37.93 | D |
| ATOM | 13253 | CG2 | VAL | D | 303 | 52.849 | 39.788 | −51.491 | 1.00 | 37.93 | D |
| ATOM | 13254 | C | VAL | D | 303 | 52.855 | 36.543 | −49.483 | 1.00 | 44.08 | D |
| ATOM | 13255 | O | VAL | D | 303 | 52.486 | 35.445 | −49.918 | 1.00 | 44.08 | D |
| ATOM | 13256 | N | LYS | D | 304 | 53.734 | 36.678 | −48.486 | 1.00 | 61.07 | D |
| ATOM | 13257 | CA | LYS | D | 304 | 54.332 | 35.520 | −47.820 | 1.00 | 61.07 | D |
| ATOM | 13258 | CB | LYS | D | 304 | 55.112 | 35.949 | −46.581 | 1.00 | 77.60 | D |
| ATOM | 13259 | CG | LYS | D | 304 | 56.514 | 36.432 | −46.826 | 1.00 | 59.52 | D |
| ATOM | 13260 | CD | LYS | D | 304 | 57.135 | 36.869 | −45.504 | 1.00 | 59.52 | D |
| ATOM | 13261 | CE | LYS | D | 304 | 58.595 | 37.257 | −45.673 | 1.00 | 59.52 | D |
| ATOM | 13262 | NZ | LYS | D | 304 | 59.171 | 37.808 | −44.418 | 1.00 | 59.52 | D |
| ATOM | 13263 | C | LYS | D | 304 | 53.252 | 34.536 | −47.382 | 1.00 | 61.07 | D |
| ATOM | 13264 | O | LYS | D | 304 | 53.359 | 33.330 | −47.599 | 1.00 | 61.07 | D |
| ATOM | 13265 | N | ALA | D | 305 | 52.202 | 35.062 | −46.772 | 1.00 | 49.25 | D |
| ATOM | 13266 | CA | ALA | D | 305 | 51.131 | 34.229 | −46.277 | 1.00 | 49.25 | D |
| ATOM | 13267 | CB | ALA | D | 305 | 50.259 | 35.037 | −45.365 | 1.00 | 27.25 | D |
| ATOM | 13268 | C | ALA | D | 305 | 50.282 | 33.572 | −47.352 | 1.00 | 49.25 | D |
| ATOM | 13269 | O | ALA | D | 305 | 50.056 | 32.361 | −47.315 | 1.00 | 49.25 | D |
| ATOM | 13270 | N | TYR | D | 306 | 49.798 | 34.357 | −48.308 | 1.00 | 49.34 | D |
| ATOM | 13271 | CA | TYR | D | 306 | 48.953 | 33.806 | −49.358 | 1.00 | 49.34 | D |
| ATOM | 13272 | CB | TYR | D | 306 | 48.239 | 34.932 | −50.102 | 1.00 | 55.36 | D |
| ATOM | 13273 | CG | TYR | D | 306 | 47.239 | 35.670 | −49.256 | 1.00 | 50.05 | D |
| ATOM | 13274 | CD1 | TYR | D | 306 | 46.950 | 37.016 | −49.491 | 1.00 | 50.05 | D |
| ATOM | 13275 | CE1 | TYR | D | 306 | 46.025 | 37.701 | −48.719 | 1.00 | 50.05 | D |
| ATOM | 13276 | CD2 | TYR | D | 306 | 46.574 | 35.026 | −48.227 | 1.00 | 50.05 | D |
| ATOM | 13277 | CE2 | TYR | D | 306 | 45.645 | 35.698 | −47.450 | 1.00 | 50.05 | D |
| ATOM | 13278 | CZ | TYR | D | 306 | 45.376 | 37.034 | −47.699 | 1.00 | 50.05 | D |
| ATOM | 13279 | OH | TYR | D | 306 | 44.457 | 37.684 | −46.910 | 1.00 | 50.05 | D |
| ATOM | 13280 | C | TYR | D | 306 | 49.702 | 32.939 | −50.346 | 1.00 | 49.34 | D |
| ATOM | 13281 | O | TYR | D | 306 | 49.290 | 31.819 | −50.616 | 1.00 | 49.34 | D |
| ATOM | 13282 | N | LEU | D | 307 | 50.798 | 33.455 | −50.889 | 1.00 | 46.63 | D |
| ATOM | 13283 | CA | LEU | D | 307 | 51.577 | 32.711 | −51.872 | 1.00 | 46.63 | D |
| ATOM | 13284 | CB | LEU | D | 307 | 52.394 | 33.662 | −52.738 | 1.00 | 59.99 | D |
| ATOM | 13285 | CG | LEU | D | 307 | 53.392 | 33.002 | −53.694 | 1.00 | 28.12 | D |
| ATOM | 13286 | CD1 | LEU | D | 307 | 52.681 | 32.097 | −54.684 | 1.00 | 28.12 | D |
| ATOM | 13287 | CD2 | LEU | D | 307 | 54.165 | 34.100 | −54.423 | 1.00 | 28.12 | D |
| ATOM | 13288 | C | LEU | D | 307 | 52.505 | 31.707 | −51.218 | 1.00 | 46.63 | D |
| ATOM | 13289 | O | LEU | D | 307 | 52.515 | 30.532 | −51.587 | 1.00 | 46.63 | D |
| ATOM | 13290 | N | GLY | D | 308 | 53.291 | 32.172 | −50.253 | 1.00 | 42.70 | D |
| ATOM | 13291 | CA | GLY | D | 308 | 54.199 | 31.282 | −49.567 | 1.00 | 42.70 | D |
| ATOM | 13292 | C | GLY | D | 308 | 53.458 | 30.138 | −48.893 | 1.00 | 42.70 | D |
| ATOM | 13293 | O | GLY | D | 308 | 53.845 | 28.976 | −49.030 | 1.00 | 42.70 | D |
| ATOM | 13294 | N | GLY | D | 309 | 52.387 | 30.453 | −48.171 | 1.00 | 43.18 | D |
| ATOM | 13295 | CA | GLY | D | 309 | 51.643 | 29.416 | −47.477 | 1.00 | 43.18 | D |
| ATOM | 13296 | C | GLY | D | 309 | 51.092 | 28.339 | −48.389 | 1.00 | 43.18 | D |
| ATOM | 13297 | O | GLY | D | 309 | 51.245 | 27.137 | −48.141 | 1.00 | 43.18 | D |
| ATOM | 13298 | N | PHE | D | 310 | 50.445 | 28.779 | −49.461 | 1.00 | 41.87 | D |
| ATOM | 13299 | CA | PHE | D | 310 | 49.849 | 27.871 | −50.424 | 1.00 | 41.87 | D |
| ATOM | 13300 | CB | PHE | D | 310 | 49.214 | 28.675 | −51.553 | 1.00 | 39.33 | D |
| ATOM | 13301 | CG | PHE | D | 310 | 48.351 | 27.859 | −52.466 | 1.00 | 35.16 | D |
| ATOM | 13302 | CD1 | PHE | D | 310 | 47.215 | 27.212 | −51.980 | 1.00 | 35.16 | D |
| ATOM | 13303 | CD2 | PHE | D | 310 | 48.656 | 27.762 | −53.816 | 1.00 | 35.16 | D |
| ATOM | 13304 | CE1 | PHE | D | 310 | 46.388 | 26.477 | −52.827 | 1.00 | 35.16 | D |
| ATOM | 13305 | CE2 | PHE | D | 310 | 47.839 | 27.031 | −54.680 | 1.00 | 35.16 | D |
| ATOM | 13306 | CZ | PHE | D | 310 | 46.699 | 26.386 | −54.180 | 1.00 | 35.16 | D |
| ATOM | 13307 | C | PHE | D | 310 | 50.909 | 26.944 | −50.996 | 1.00 | 41.87 | D |
| ATOM | 13308 | O | PHE | D | 310 | 50.694 | 25.736 | −51.125 | 1.00 | 41.87 | D |
| ATOM | 13309 | N | GLU | D | 311 | 52.050 | 27.534 | −51.341 | 1.00 | 33.59 | D |
| ATOM | 13310 | CA | GLU | D | 311 | 53.161 | 26.800 | −51.917 | 1.00 | 33.59 | D |
| ATOM | 13311 | CB | GLU | D | 311 | 54.194 | 27.767 | −52.490 | 1.00 | 93.21 | D |
| ATOM | 13312 | CG | GLU | D | 311 | 55.049 | 27.143 | −53.571 | 1.00 | 77.44 | D |
| ATOM | 13313 | CD | GLU | D | 311 | 56.186 | 28.036 | −54.020 | 1.00 | 77.44 | D |
| ATOM | 13314 | OE1 | GLU | D | 311 | 55.987 | 29.265 | −54.126 | 1.00 | 77.44 | D |
| ATOM | 13315 | OE2 | GLU | D | 311 | 57.282 | 27.500 | −54.286 | 1.00 | 77.44 | D |
| ATOM | 13316 | C | GLU | D | 311 | 53.825 | 25.897 | −50.889 | 1.00 | 33.59 | D |
| ATOM | 13317 | O | GLU | D | 311 | 54.290 | 24.805 | −51.213 | 1.00 | 33.59 | D |
| ATOM | 13318 | N | TYR | D | 312 | 53.853 | 26.339 | −49.642 | 1.00 | 40.87 | D |
| ATOM | 13319 | CA | TYR | D | 312 | 54.489 | 25.555 | −48.600 | 1.00 | 40.87 | D |
| ATOM | 13320 | CB | TYR | D | 312 | 54.687 | 26.380 | −47.339 | 1.00 | 57.24 | D |
| ATOM | 13321 | CG | TYR | D | 312 | 55.420 | 25.624 | −46.265 | 1.00 | 51.94 | D |
| ATOM | 13322 | CD1 | TYR | D | 312 | 56.780 | 25.374 | −46.374 | 1.00 | 51.94 | D |
| ATOM | 13323 | CE1 | TYR | D | 312 | 57.460 | 24.640 | −45.394 | 1.00 | 51.94 | D |
| ATOM | 13324 | CD2 | TYR | D | 312 | 54.744 | 25.124 | −45.147 | 1.00 | 51.94 | D |
| ATOM | 13325 | CE2 | TYR | D | 312 | 55.409 | 24.388 | −44.158 | 1.00 | 51.94 | D |
| ATOM | 13326 | CZ | TYR | D | 312 | 56.764 | 24.151 | −44.289 | 1.00 | 51.94 | D |
| ATOM | 13327 | OH | TYR | D | 312 | 57.423 | 23.423 | −43.320 | 1.00 | 51.94 | D |
| ATOM | 13328 | C | TYR | D | 312 | 53.726 | 24.305 | −48.236 | 1.00 | 40.87 | D |
| ATOM | 13329 | O | TYR | D | 312 | 54.325 | 23.276 | −47.958 | 1.00 | 40.87 | D |
| ATOM | 13330 | N | MET | D | 313 | 52.408 | 24.376 | −48.222 | 1.00 | 43.69 | D |
| ATOM | 13331 | CA | MET | D | 313 | 51.645 | 23.192 | −47.870 | 1.00 | 43.69 | D |
| ATOM | 13332 | CB | MET | D | 313 | 51.727 | 22.966 | −46.364 | 1.00 | 55.91 | D |
| ATOM | 13333 | CG | MET | D | 313 | 51.007 | 21.729 | −45.897 | 1.00 | 55.76 | D |
| ATOM | 13334 | SD | MET | D | 313 | 50.996 | 21.629 | −44.128 | 1.00 | 55.76 | D |
| ATOM | 13335 | CE | MET | D | 313 | 52.770 | 21.629 | −43.812 | 1.00 | 55.76 | D |
| ATOM | 13336 | C | MET | D | 313 | 50.177 | 23.241 | −48.295 | 1.00 | 43.69 | D |
| ATOM | 13337 | O | MET | D | 313 | 49.585 | 22.210 | −48.646 | 1.00 | 43.69 | D |
| ATOM | 13338 | N | GLY | D | 314 | 49.597 | 24.438 | −48.257 | 1.00 | 33.20 | D |
| ATOM | 13339 | CA | GLY | D | 314 | 48.203 | 24.599 | −48.619 | 1.00 | 33.20 | D |
| ATOM | 13340 | C | GLY | D | 314 | 47.775 | 23.852 | −49.866 | 1.00 | 33.20 | D |
| ATOM | 13341 | O | GLY | D | 314 | 46.822 | 23.058 | −49.845 | 1.00 | 33.20 | D |
| ATOM | 13342 | N | CYS | D | 315 | 48.485 | 24.100 | −50.959 | 1.00 | 45.89 | D |
| ATOM | 13343 | CA | CYS | D | 315 | 48.164 | 23.457 | −52.217 | 1.00 | 45.89 | D |
| ATOM | 13344 | CB | CYS | D | 315 | 49.128 | 23.929 | −53.295 | 1.00 | 64.49 | D |
| ATOM | 13345 | SG | CYS | D | 315 | 48.592 | 23.501 | −54.946 | 1.00 | 69.86 | D |
| ATOM | 13346 | C | CYS | D | 315 | 48.183 | 21.934 | −52.142 | 1.00 | 45.89 | D |
| ATOM | 13347 | O | CYS | D | 315 | 47.208 | 21.280 | −52.513 | 1.00 | 45.89 | D |
| ATOM | 13348 | N | SER | D | 316 | 49.286 | 21.364 | −51.661 | 1.00 | 48.57 | D |
| ATOM | 13349 | CA | SER | D | 316 | 49.393 | 19.910 | −51.565 | 1.00 | 48.57 | D |
| ATOM | 13350 | CB | SER | D | 316 | 50.703 | 19.513 | −50.915 | 1.00 | 36.46 | D |
| ATOM | 13351 | OG | SER | D | 316 | 50.705 | 19.888 | −49.553 | 1.00 | 41.96 | D |
| ATOM | 13352 | C | SER | D | 316 | 48.250 | 19.284 | −50.779 | 1.00 | 48.57 | D |
| ATOM | 13353 | O | SER | D | 316 | 47.913 | 18.132 | −50.999 | 1.00 | 48.57 | D |
| ATOM | 13354 | N | LEU | D | 317 | 47.666 | 20.027 | −49.848 | 1.00 | 42.70 | D |
| ATOM | 13355 | CA | LEU | D | 317 | 46.552 | 19.496 | −49.078 | 1.00 | 42.70 | D |
| ATOM | 13356 | CB | LEU | D | 317 | 46.406 | 20.253 | −47.751 | 1.00 | 41.81 | D |
| ATOM | 13357 | CG | LEU | D | 317 | 47.478 | 19.994 | −46.687 | 1.00 | 34.32 | D |
| ATOM | 13358 | CD1 | LEU | D | 317 | 47.307 | 20.919 | −45.507 | 1.00 | 34.32 | D |
| ATOM | 13359 | CD2 | LEU | D | 317 | 47.382 | 18.556 | −46.243 | 1.00 | 34.32 | D |
| ATOM | 13360 | C | LEU | D | 317 | 45.269 | 19.641 | −49.904 | 1.00 | 42.70 | D |
| ATOM | 13361 | O | LEU | D | 317 | 44.447 | 18.713 | −50.002 | 1.00 | 42.70 | D |
| ATOM | 13362 | N | ALA | D | 318 | 45.112 | 20.807 | −50.522 | 1.00 | 35.61 | D |
| ATOM | 13363 | CA | ALA | D | 318 | 43.923 | 21.081 | −51.309 | 1.00 | 35.61 | D |
| ATOM | 13364 | CB | ALA | D | 318 | 43.952 | 22.520 | −51.812 | 1.00 | 31.04 | D |
| ATOM | 13365 | C | ALA | D | 318 | 43.757 | 20.126 | −52.474 | 1.00 | 35.61 | D |
| ATOM | 13366 | O | ALA | D | 318 | 42.685 | 19.565 | −52.690 | 1.00 | 35.61 | D |
| ATOM | 13367 | N | ILE | D | 319 | 44.824 | 19.937 | −53.229 | 1.00 | 39.02 | D |
| ATOM | 13368 | CA | ILE | D | 319 | 44.751 | 19.062 | −54.392 | 1.00 | 39.02 | D |
| ATOM | 13369 | CB | ILE | D | 319 | 46.146 | 18.779 | −54.981 | 1.00 | 55.98 | D |
| ATOM | 13370 | CG2 | ILE | D | 319 | 46.923 | 17.843 | −54.079 | 1.00 | 30.54 | D |
| ATOM | 13371 | CG1 | ILE | D | 319 | 45.987 | 18.162 | −56.368 | 1.00 | 30.54 | D |
| ATOM | 13372 | CD1 | ILE | D | 319 | 47.298 | 17.978 | −57.131 | 1.00 | 30.54 | D |
| ATOM | 13373 | C | ILE | D | 319 | 44.065 | 17.734 | −54.083 | 1.00 | 39.02 | D |
| ATOM | 13374 | O | ILE | D | 319 | 43.418 | 17.171 | −54.949 | 1.00 | 39.02 | D |
| ATOM | 13375 | N | LEU | D | 320 | 44.193 | 17.246 | −52.849 | 1.00 | 39.80 | D |
| ATOM | 13376 | CA | LEU | D | 320 | 43.564 | 15.980 | −52.480 | 1.00 | 39.80 | D |
| ATOM | 13377 | CB | LEU | D | 320 | 44.147 | 15.461 | −51.156 | 1.00 | 66.49 | D |
| ATOM | 13378 | CG | LEU | D | 320 | 45.637 | 15.076 | −51.210 | 1.00 | 44.33 | D |
| ATOM | 13379 | CD1 | LEU | D | 320 | 46.089 | 14.564 | −49.858 | 1.00 | 44.33 | D |
| ATOM | 13380 | CD2 | LEU | D | 320 | 45.880 | 14.019 | −52.291 | 1.00 | 44.33 | D |
| ATOM | 13381 | C | LEU | D | 320 | 42.036 | 16.085 | −52.395 | 1.00 | 39.80 | D |
| ATOM | 13382 | O | LEU | D | 320 | 41.326 | 15.118 | −52.686 | 1.00 | 39.80 | D |
| ATOM | 13383 | N | ASP | D | 321 | 41.533 | 17.260 | −52.012 | 1.00 | 46.17 | D |
| ATOM | 13384 | CA | ASP | D | 321 | 40.092 | 17.462 | −51.922 | 1.00 | 46.17 | D |
| ATOM | 13385 | CB | ASP | D | 321 | 39.765 | 18.766 | −51.183 | 1.00 | 94.54 | D |
| ATOM | 13386 | CG | ASP | D | 321 | 40.051 | 18.687 | −49.690 | 1.00 | 73.54 | D |
| ATOM | 13387 | OD1 | ASP | D | 321 | 39.697 | 17.658 | −49.075 | 1.00 | 73.54 | D |
| ATOM | 13388 | OD2 | ASP | D | 321 | 40.612 | 19.667 | −49.135 | 1.00 | 73.54 | D |
| ATOM | 13389 | C | ASP | D | 321 | 39.507 | 17.502 | −53.335 | 1.00 | 46.17 | D |
| ATOM | 13390 | O | ASP | D | 321 | 38.421 | 16.982 | −53.585 | 1.00 | 46.17 | D |
| ATOM | 13391 | N | TYR | D | 322 | 40.236 | 18.115 | −54.264 | 1.00 | 49.07 | D |
| ATOM | 13392 | CA | TYR | D | 322 | 39.777 | 18.202 | −55.645 | 1.00 | 49.07 | D |
| ATOM | 13393 | CB | TYR | D | 322 | 40.770 | 18.977 | −56.517 | 1.00 | 43.94 | D |
| ATOM | 13394 | CG | TYR | D | 322 | 40.745 | 20.483 | −56.344 | 1.00 | 34.65 | D |
| ATOM | 13395 | CD1 | TYR | D | 322 | 41.220 | 21.080 | −55.177 | 1.00 | 34.65 | D |
| ATOM | 13396 | CE1 | TYR | D | 322 | 41.227 | 22.472 | −55.028 | 1.00 | 34.65 | D |
| ATOM | 13397 | CD2 | TYR | D | 322 | 40.271 | 21.315 | −57.361 | 1.00 | 34.65 | D |
| ATOM | 13398 | CE2 | TYR | D | 322 | 40.278 | 22.711 | −57.223 | 1.00 | 34.65 | D |
| ATOM | 13399 | CZ | TYR | D | 322 | 40.761 | 23.281 | −56.054 | 1.00 | 34.65 | D |
| ATOM | 13400 | OH | TYR | D | 322 | 40.818 | 24.647 | −55.919 | 1.00 | 34.65 | D |
| ATOM | 13401 | C | TYR | D | 322 | 39.648 | 16.796 | −56.187 | 1.00 | 49.07 | D |
| ATOM | 13402 | O | TYR | D | 322 | 38.626 | 16.419 | −56.770 | 1.00 | 49.07 | D |
| ATOM | 13403 | N | GLN | D | 323 | 40.701 | 16.016 | −55.986 | 1.00 | 47.42 | D |
| ATOM | 13404 | CA | GLN | D | 323 | 40.726 | 14.650 | −56.456 | 1.00 | 47.42 | D |
| ATOM | 13405 | CB | GLN | D | 323 | 41.980 | 13.954 | −55.964 | 1.00 | 77.41 | D |
| ATOM | 13406 | CG | GLN | D | 323 | 42.067 | 12.536 | −56.424 | 1.00 | 51.80 | D |
| ATOM | 13407 | CD | GLN | D | 323 | 43.369 | 11.889 | −56.036 | 1.00 | 51.80 | D |
| ATOM | 13408 | OE1 | GLN | D | 323 | 43.784 | 11.933 | −54.870 | 1.00 | 51.80 | D |
| ATOM | 13409 | NE2 | GLN | D | 323 | 44.025 | 11.271 | −57.011 | 1.00 | 51.80 | D |
| ATOM | 13410 | C | GLN | D | 323 | 39.485 | 13.892 | −56.000 | 1.00 | 47.42 | D |
| ATOM | 13411 | O | GLN | D | 323 | 38.850 | 13.230 | −56.807 | 1.00 | 47.42 | D |
| ATOM | 13412 | N | LYS | D | 324 | 39.136 | 13.994 | −54.718 | 1.00 | 38.54 | D |
| ATOM | 13413 | CA | LYS | D | 324 | 37.952 | 13.310 | −54.187 | 1.00 | 38.54 | D |
| ATOM | 13414 | CB | LYS | D | 324 | 37.734 | 13.648 | −52.710 | 1.00 | 81.92 | D |
| ATOM | 13415 | CG | LYS | D | 324 | 38.628 | 12.928 | −51.747 | 1.00 | 71.35 | D |
| ATOM | 13416 | CD | LYS | D | 324 | 38.133 | 13.121 | −50.328 | 1.00 | 71.35 | D |
| ATOM | 13417 | CE | LYS | D | 324 | 39.001 | 12.369 | −49.328 | 1.00 | 71.35 | D |
| ATOM | 13418 | NZ | LYS | D | 324 | 38.464 | 12.437 | −47.928 | 1.00 | 71.35 | D |
| ATOM | 13419 | C | LYS | D | 324 | 36.682 | 13.691 | −54.950 | 1.00 | 38.54 | D |
| ATOM | 13420 | O | LYS | D | 324 | 35.920 | 12.826 | −55.391 | 1.00 | 38.54 | D |
| ATOM | 13421 | N | MET | D | 325 | 36.447 | 14.989 | −55.097 | 1.00 | 52.31 | D |
| ATOM | 13422 | CA | MET | D | 325 | 35.247 | 15.443 | −55.768 | 1.00 | 52.31 | D |
| ATOM | 13423 | CB | MET | D | 325 | 34.989 | 16.904 | −55.451 | 1.00 | 66.27 | D |
| ATOM | 13424 | CG | MET | D | 325 | 34.677 | 17.134 | −54.016 | 1.00 | 54.83 | D |
| ATOM | 13425 | SD | MET | D | 325 | 34.384 | 18.838 | −53.701 | 1.00 | 54.83 | D |
| ATOM | 13426 | CE | MET | D | 325 | 36.015 | 19.361 | −53.064 | 1.00 | 54.83 | D |
| ATOM | 13427 | C | MET | D | 325 | 35.290 | 15.253 | −57.268 | 1.00 | 52.31 | D |
| ATOM | 13428 | O | MET | D | 325 | 34.265 | 15.356 | −57.941 | 1.00 | 52.31 | D |
| ATOM | 13429 | N | ALA | D | 326 | 36.464 | 14.964 | −57.808 | 1.00 | 38.89 | D |
| ATOM | 13430 | CA | ALA | D | 326 | 36.550 | 14.791 | −59.246 | 1.00 | 36.89 | D |
| ATOM | 13431 | CB | ALA | D | 326 | 37.872 | 15.369 | −59.767 | 1.00 | 26.18 | D |
| ATOM | 13432 | C | ALA | D | 326 | 36.424 | 13.336 | −59.657 | 1.00 | 38.89 | D |
| ATOM | 13433 | O | ALA | D | 326 | 36.024 | 13.048 | −60.785 | 1.00 | 38.89 | D |
| ATOM | 13434 | N | LYS | D | 327 | 36.756 | 12.435 | −58.733 | 1.00 | 99.15 | D |
| ATOM | 13435 | CA | LYS | D | 327 | 36.748 | 10.999 | −58.982 | 1.00 | 99.15 | D |
| ATOM | 13436 | CB | LYS | D | 327 | 36.338 | 10.220 | −57.735 | 1.00 | 157.88 | D |
| ATOM | 13437 | CG | LYS | D | 327 | 36.619 | 8.728 | −57.879 | 1.00 | 76.17 | D |
| ATOM | 13438 | CD | LYS | D | 327 | 36.343 | 7.965 | −56.595 | 1.00 | 76.17 | D |
| ATOM | 13439 | CE | LYS | D | 327 | 36.741 | 6.497 | −56.721 | 1.00 | 76.17 | D |
| ATOM | 13440 | NZ | LYS | D | 327 | 36.447 | 5.726 | −55.475 | 1.00 | 76.17 | D |
| ATOM | 13441 | C | LYS | D | 327 | 35.900 | 10.535 | −60.147 | 1.00 | 99.15 | D |
| ATOM | 13442 | O | LYS | D | 327 | 36.426 | 10.052 | −61.152 | 1.00 | 99.15 | D |
| ATOM | 13443 | N | GLY | D | 328 | 34.590 | 10.682 | −60.037 | 1.00 | 39.83 | D |
| ATOM | 13444 | CA | GLY | D | 328 | 33.760 | 10.216 | −61.133 | 1.00 | 39.83 | D |
| ATOM | 13445 | C | GLY | D | 328 | 33.465 | 11.164 | −62.290 | 1.00 | 39.83 | D |
| ATOM | 13446 | O | GLY | D | 328 | 33.174 | 10.718 | −63.397 | 1.00 | 39.83 | D |
| ATOM | 13447 | N | LYS | D | 329 | 33.556 | 12.472 | −62.065 | 1.00 | 47.04 | D |
| ATOM | 13448 | CA | LYS | D | 329 | 33.221 | 13.421 | −63.118 | 1.00 | 47.04 | D |
| ATOM | 13449 | CB | LYS | D | 329 | 32.553 | 14.639 | −62.478 | 1.00 | 176.74 | D |
| ATOM | 13450 | CG | LYS | D | 329 | 31.225 | 14.315 | −61.814 | 1.00 | 176.74 | D |
| ATOM | 13451 | CD | LYS | D | 329 | 30.414 | 15.573 | −61.615 | 1.00 | 176.74 | D |
| ATOM | 13452 | CE | LYS | D | 329 | 29.047 | 15.290 | −61.029 | 1.00 | 66.50 | D |
| ATOM | 13453 | NZ | LYS | D | 329 | 28.279 | 16.575 | −60.918 | 1.00 | 66.50 | D |
| ATOM | 13454 | C | LYS | D | 329 | 34.250 | 13.884 | −64.158 | 1.00 | 47.04 | D |
| ATOM | 13455 | O | LYS | D | 329 | 34.014 | 14.863 | −64.852 | 1.00 | 47.04 | D |
| ATOM | 13456 | N | ILE | D | 330 | 35.378 | 13.199 | −64.301 | 1.00 | 36.69 | D |
| ATOM | 13457 | CA | ILE | D | 330 | 36.371 | 13.620 | −65.310 | 1.00 | 36.69 | D |
| ATOM | 13458 | CB | ILE | D | 330 | 37.539 | 14.457 | −64.685 | 1.00 | 33.98 | D |
| ATOM | 13459 | CG2 | ILE | D | 330 | 36.980 | 15.668 | −63.917 | 1.00 | 34.90 | D |
| ATOM | 13460 | CG1 | ILE | D | 330 | 38.388 | 13.584 | −63.762 | 1.00 | 34.90 | D |
| ATOM | 13461 | CD1 | ILE | D | 330 | 39.702 | 14.209 | −63.431 | 1.00 | 34.90 | D |
| ATOM | 13462 | C | ILE | D | 330 | 36.960 | 12.399 | −66.024 | 1.00 | 36.69 | D |
| ATOM | 13463 | O | ILE | D | 330 | 36.723 | 11.265 | −65.604 | 1.00 | 36.69 | D |
| ATOM | 13464 | N | PRO | D | 331 | 37.725 | 12.608 | −67.114 | 1.00 | 35.18 | D |
| ATOM | 13465 | CD | PRO | D | 331 | 38.132 | 13.904 | −67.674 | 1.00 | 41.22 | D |
| ATOM | 13466 | CA | PRO | D | 331 | 38.344 | 11.516 | −67.883 | 1.00 | 35.18 | D |
| ATOM | 13467 | CB | PRO | D | 331 | 39.366 | 12.237 | −68.736 | 1.00 | 185.20 | D |
| ATOM | 13468 | CG | PRO | D | 331 | 38.694 | 13.481 | −69.023 | 1.00 | 41.22 | D |
| ATOM | 13469 | C | PRO | D | 331 | 38.999 | 10.502 | −66.972 | 1.00 | 35.18 | D |
| ATOM | 13470 | O | PRO | D | 331 | 39.703 | 10.861 | −66.017 | 1.00 | 35.18 | D |
| ATOM | 13471 | N | LYS | D | 332 | 38.771 | 9.226 | −67.251 | 1.00 | 45.78 | D |
| ATOM | 13472 | CA | LYS | D | 332 | 39.352 | 8.164 | −66.435 | 1.00 | 46.40 | D |
| ATOM | 13473 | CB | LYS | D | 332 | 38.696 | 6.833 | −66.778 | 1.00 | 95.87 | D |
| ATOM | 13474 | CG | LYS | D | 332 | 38.880 | 6.463 | −68.226 | 1.00 | 67.41 | D |
| ATOM | 13475 | CD | LYS | D | 332 | 38.691 | 4.986 | −68.444 | 1.00 | 67.41 | D |
| ATOM | 13476 | CE | LYS | D | 332 | 39.099 | 4.589 | −69.851 | 1.00 | 67.41 | D |
| ATOM | 13477 | NZ | LYS | D | 332 | 39.023 | 3.108 | −70.026 | 1.00 | 67.41 | D |
| ATOM | 13478 | C | LYS | D | 332 | 40.857 | 8.067 | −66.695 | 1.00 | 45.47 | D |
| ATOM | 13479 | O | LYS | D | 332 | 41.343 | 8.511 | −67.737 | 1.00 | 43.56 | D |
| ATOM | 13480 | N | ASP | D | 333 | 41.593 | 7.487 | −65.750 | 1.00 | 50.62 | D |
| ATOM | 13481 | CA | ASP | D | 333 | 43.031 | 7.323 | −65.917 | 1.00 | 50.08 | D |
| ATOM | 13482 | CB | ASP | D | 333 | 43.649 | 6.720 | −64.660 | 1.00 | 131.68 | D |
| ATOM | 13483 | CG | ASP | D | 333 | 44.080 | 7.770 | −63.666 | 1.00 | 99.27 | D |
| ATOM | 13484 | OD1 | ASP | D | 333 | 43.372 | 8.789 | −63.526 | 1.00 | 99.27 | D |
| ATOM | 13485 | OD2 | ASP | D | 333 | 45.125 | 7.575 | −63.012 | 1.00 | 99.27 | D |
| ATOM | 13486 | C | ASP | D | 333 | 43.294 | 6.406 | −67.108 | 1.00 | 53.93 | D |
| ATOM | 13487 | O | ASP | D | 333 | 42.580 | 5.429 | −67.329 | 1.00 | 53.58 | D |
| ATOM | 13488 | N | THR | D | 334 | 44.321 | 6.727 | −67.879 | 1.00 | 42.54 | D |
| ATOM | 13489 | CA | THR | D | 334 | 44.665 | 5.921 | −69.037 | 1.00 | 42.26 | D |
| ATOM | 13490 | CB | THR | D | 334 | 44.161 | 6.580 | −70.340 | 1.00 | 68.31 | D |
| ATOM | 13491 | OG1 | THR | D | 334 | 42.743 | 6.435 | −70.419 | 1.00 | 54.21 | D |
| ATOM | 13492 | CG2 | THR | D | 334 | 44.792 | 5.944 | −71.560 | 1.00 | 54.21 | D |
| ATOM | 13493 | C | THR | D | 334 | 46.169 | 5.728 | −69.117 | 1.00 | 48.70 | D |
| ATOM | 13494 | O | THR | D | 334 | 46.931 | 6.695 | −69.209 | 1.00 | 46.06 | D |
| ATOM | 13495 | N | THR | D | 335 | 46.589 | 4.468 | −69.087 | 1.00 | 46.52 | D |
| ATOM | 13496 | CA | THR | D | 335 | 48.003 | 4.141 | −69.158 | 1.00 | 46.31 | D |
| ATOM | 13497 | CB | THR | D | 335 | 48.240 | 2.739 | −68.682 | 1.00 | 58.95 | D |
| ATOM | 13498 | OG1 | THR | D | 335 | 47.526 | 2.534 | −67.460 | 1.00 | 48.31 | D |
| ATOM | 13499 | CG2 | THR | D | 335 | 49.709 | 2.532 | −68.435 | 1.00 | 48.31 | D |
| ATOM | 13500 | C | THR | D | 335 | 48.588 | 4.286 | −70.555 | 1.00 | 47.17 | D |
| ATOM | 13501 | O | THR | D | 335 | 48.023 | 3.822 | −71.539 | 1.00 | 48.52 | D |
| ATOM | 13502 | N | PRO | D | 336 | 49.754 | 4.915 | −70.656 | 1.00 | 45.40 | D |
| ATOM | 13503 | CD | PRO | D | 336 | 50.565 | 5.515 | −69.583 | 1.00 | 31.16 | D |
| ATOM | 13504 | CA | PRO | D | 336 | 50.385 | 5.112 | −71.957 | 1.00 | 44.98 | D |
| ATOM | 13505 | CB | PRO | D | 336 | 51.277 | 6.312 | −71.707 | 1.00 | 40.02 | D |
| ATOM | 13506 | CG | PRO | D | 336 | 51.812 | 5.970 | −70.335 | 1.00 | 31.16 | D |
| ATOM | 13507 | C | PRO | D | 336 | 51.197 | 3.925 | −72.456 | 1.00 | 45.02 | D |
| ATOM | 13508 | O | PRO | D | 336 | 51.386 | 2.934 | −71.754 | 1.00 | 45.65 | D |
| ATOM | 13509 | N | THR | D | 337 | 51.680 | 4.069 | −73.684 | 1.00 | 56.57 | D |
| ATOM | 13510 | CA | THR | D | 337 | 52.513 | 3.090 | −74.349 | 1.00 | 60.03 | D |
| ATOM | 13511 | CB | THR | D | 337 | 51.960 | 2.765 | −75.738 | 1.00 | 112.37 | D |
| ATOM | 13512 | OG1 | THR | D | 337 | 50.783 | 1.968 | −75.603 | 1.00 | 63.26 | D |
| ATOM | 13513 | CG2 | THR | D | 337 | 52.979 | 2.017 | −76.565 | 1.00 | 63.26 | D |
| ATOM | 13514 | C | THR | D | 337 | 53.835 | 3.819 | −74.508 | 1.00 | 67.60 | D |
| ATOM | 13515 | O | THR | D | 337 | 53.865 | 4.965 | −74.963 | 1.00 | 58.98 | D |
| ATOM | 13516 | N | VAL | D | 338 | 54.930 | 3.178 | −74.131 | 1.00 | 47.11 | D |
| ATOM | 13517 | CA | VAL | D | 338 | 56.218 | 3.841 | −74.259 | 1.00 | 53.97 | D |
| ATOM | 13518 | CB | VAL | D | 338 | 56.903 | 4.017 | −72.882 | 1.00 | 51.74 | D |
| ATOM | 13519 | CG1 | VAL | D | 338 | 58.121 | 4.938 | −73.018 | 1.00 | 51.36 | D |
| ATOM | 13520 | CG2 | VAL | D | 338 | 55.915 | 4.587 | −71.886 | 1.00 | 51.36 | D |
| ATOM | 13521 | C | VAL | D | 338 | 57.155 | 3.082 | −75.176 | 1.00 | 58.44 | D |
| ATOM | 13522 | O | VAL | D | 338 | 57.107 | 1.856 | −75.236 | 1.00 | 58.27 | D |
| ATOM | 13523 | N | LYS | D | 339 | 57.992 | 3.832 | −75.891 | 1.00 | 68.28 | D |
| ATOM | 13524 | CA | LYS | D | 339 | 58.978 | 3.285 | −76.816 | 1.00 | 66.16 | D |
| ATOM | 13525 | CB | LYS | D | 339 | 58.357 | 3.083 | −78.203 | 1.00 | 130.80 | D |
| ATOM | 13526 | CG | LYS | D | 339 | 57.251 | 2.040 | −78.227 | 1.00 | 99.11 | D |
| ATOM | 13527 | CD | LYS | D | 339 | 56.172 | 2.362 | −79.253 | 1.00 | 99.11 | D |
| ATOM | 13528 | CE | LYS | D | 339 | 54.989 | 1.403 | −79.119 | 1.00 | 99.11 | D |
| ATOM | 13529 | NZ | LYS | D | 339 | 53.824 | 1.807 | −79.952 | 1.00 | 99.11 | D |
| ATOM | 13530 | C | LYS | D | 339 | 60.105 | 4.301 | −76.890 | 1.00 | 75.15 | D |
| ATOM | 13531 | O | LYS | D | 339 | 59.857 | 5.501 | −77.018 | 1.00 | 67.81 | D |
| ATOM | 13532 | N | VAL | D | 340 | 61.340 | 3.830 | −76.772 | 1.00 | 68.34 | D |
| ATOM | 13533 | CA | VAL | D | 340 | 62.495 | 4.720 | −76.855 | 1.00 | 65.77 | D |
| ATOM | 13534 | CB | VAL | D | 340 | 63.489 | 4.474 | −75.708 | 1.00 | 50.49 | D |
| ATOM | 13535 | CG1 | VAL | D | 340 | 64.666 | 5.421 | −75.831 | 1.00 | 35.95 | D |
| ATOM | 13536 | CG2 | VAL | D | 340 | 62.811 | 4.674 | −74.389 | 1.00 | 35.95 | D |
| ATOM | 13537 | C | VAL | D | 340 | 63.210 | 4.425 | −78.167 | 1.00 | 68.34 | D |
| ATOM | 13538 | O | VAL | D | 340 | 63.579 | 3.292 | −78.409 | 1.00 | 65.19 | D |
| ATOM | 13539 | N | THR | D | 341 | 63.408 | 5.433 | −79.008 | 1.00 | 73.35 | D |
| ATOM | 13540 | CA | THR | D | 341 | 64.075 | 5.216 | −80.287 | 1.00 | 69.25 | D |
| ATOM | 13541 | CB | THR | D | 341 | 63.226 | 5.752 | −81.451 | 1.00 | 127.99 | D |
| ATOM | 13542 | OG1 | THR | D | 341 | 61.968 | 5.074 | −81.469 | 1.00 | 109.35 | D |
| ATOM | 13543 | CG2 | THR | D | 341 | 63.931 | 5.523 | −82.779 | 1.00 | 109.35 | D |
| ATOM | 13544 | C | THR | D | 341 | 65.443 | 5.880 | −80.342 | 1.00 | 71.40 | D |
| ATOM | 13545 | O | THR | D | 341 | 65.656 | 6.961 | −79.782 | 1.00 | 73.00 | D |
| ATOM | 13546 | N | GLY | D | 342 | 66.368 | 5.226 | −81.032 | 1.00 | 131.96 | D |
| ATOM | 13547 | CA | GLY | D | 342 | 67.703 | 5.769 | −81.148 | 1.00 | 136.13 | D |
| ATOM | 13548 | C | GLY | D | 342 | 68.012 | 6.276 | −82.539 | 1.00 | 135.10 | D |
| ATOM | 13549 | O | GLY | D | 342 | 68.870 | 5.721 | −83.216 | 1.00 | 129.37 | D |
| ATOM | 13550 | N | ASN | D | 343 | 67.315 | 7.323 | −82.969 | 1.00 | 80.28 | D |
| ATOM | 13551 | CA | ASN | D | 343 | 67.544 | 7.916 | −84.288 | 1.00 | 86.69 | D |
| ATOM | 13552 | CB | ASN | D | 343 | 66.475 | 8.966 | −84.599 | 1.00 | 171.53 | D |
| ATOM | 13553 | CG | ASN | D | 343 | 65.068 | 8.436 | −84.434 | 1.00 | 151.33 | D |
| ATOM | 13554 | OD1 | ASN | D | 343 | 64.659 | 7.502 | −85.124 | 1.00 | 151.33 | D |
| ATOM | 13555 | ND2 | ASN | D | 343 | 64.316 | 9.034 | −83.514 | 1.00 | 151.33 | D |
| ATOM | 13556 | C | ASN | D | 343 | 68.910 | 8.600 | −84.264 | 1.00 | 84.75 | D |
| ATOM | 13557 | O | ASN | D | 343 | 69.699 | 8.388 | −83.345 | 1.00 | 73.38 | D |
| ATOM | 13558 | N | GLU | D | 344 | 69.188 | 9.421 | −85.271 | 1.00 | 108.32 | D |
| ATOM | 13559 | CA | GLU | D | 344 | 70.454 | 10.149 | −85.332 | 1.00 | 108.32 | D |
| ATOM | 13560 | CB | GLU | D | 344 | 71.589 | 9.210 | −85.749 | 1.00 | 175.62 | D |
| ATOM | 13561 | CG | GLU | D | 344 | 72.239 | 8.519 | −84.551 | 1.00 | 155.52 | D |
| ATOM | 13562 | CD | GLU | D | 344 | 73.243 | 7.454 | −84.935 | 1.00 | 155.52 | D |
| ATOM | 13563 | OE1 | GLU | D | 344 | 72.820 | 6.367 | −85.380 | 1.00 | 155.52 | D |
| ATOM | 13564 | OE2 | GLU | D | 344 | 74.457 | 7.705 | −84.792 | 1.00 | 155.52 | D |
| ATOM | 13565 | C | GLU | D | 344 | 70.362 | 11.362 | −86.256 | 1.00 | 108.32 | D |
| ATOM | 13566 | O | GLU | D | 344 | 70.288 | 11.235 | −87.480 | 1.00 | 108.32 | D |
| ATOM | 13567 | N | LEU | D | 345 | 70.368 | 12.541 | −85.635 | 1.00 | 198.22 | D |
| ATOM | 13568 | CA | LEU | D | 345 | 70.237 | 13.817 | −86.333 | 1.00 | 198.22 | D |
| ATOM | 13569 | CB | LEU | D | 345 | 69.861 | 14.915 | −85.323 | 1.00 | 190.50 | D |
| ATOM | 13570 | CG | LEU | D | 345 | 68.540 | 14.752 | −84.547 | 1.00 | 143.29 | D |
| ATOM | 13571 | CD1 | LEU | D | 345 | 68.316 | 15.965 | −83.647 | 1.00 | 143.29 | D |
| ATOM | 13572 | CD2 | LEU | D | 345 | 67.373 | 14.598 | −85.514 | 1.00 | 143.29 | D |
| ATOM | 13573 | C | LEU | D | 345 | 71.413 | 14.284 | −87.199 | 1.00 | 198.22 | D |
| ATOM | 13574 | O | LEU | D | 345 | 71.679 | 15.481 | −87.305 | 1.00 | 198.22 | D |
| ATOM | 13575 | N | GLU | D | 346 | 72.112 | 13.337 | −87.815 | 1.00 | 147.13 | D |
| ATOM | 13576 | CA | GLU | D | 346 | 73.227 | 13.627 | −88.722 | 1.00 | 147.13 | D |
| ATOM | 13577 | CB | GLU | D | 346 | 72.695 | 14.427 | −89.909 | 1.00 | 145.32 | D |
| ATOM | 13578 | CG | GLU | D | 346 | 71.369 | 13.886 | −90.403 | 1.00 | 130.75 | D |
| ATOM | 13579 | CD | GLU | D | 346 | 71.044 | 14.310 | −91.815 | 1.00 | 130.75 | D |
| ATOM | 13580 | OE1 | GLU | D | 346 | 71.764 | 13.880 | −92.740 | 1.00 | 130.75 | D |
| ATOM | 13581 | OE2 | GLU | D | 346 | 70.070 | 15.071 | −92.002 | 1.00 | 130.75 | D |
| ATOM | 13582 | C | GLU | D | 346 | 74.508 | 14.287 | −88.194 | 1.00 | 147.13 | D |
| ATOM | 13583 | O | GLU | D | 346 | 75.533 | 13.618 | −88.062 | 1.00 | 147.13 | D |
| ATOM | 13584 | N | ASP | D | 347 | 74.466 | 15.587 | −87.909 | 1.00 | 162.59 | D |
| ATOM | 13585 | CA | ASP | D | 347 | 75.660 | 16.283 | −87.420 | 1.00 | 162.59 | D |
| ATOM | 13586 | CB | ASP | D | 347 | 75.388 | 17.786 | −87.269 | 1.00 | 173.29 | D |
| ATOM | 13587 | CG | ASP | D | 347 | 74.718 | 18.134 | −85.951 | 1.00 | 173.29 | D |
| ATOM | 13588 | OD1 | ASP | D | 347 | 73.589 | 17.659 | −85.713 | 1.00 | 173.29 | D |
| ATOM | 13589 | OD2 | ASP | D | 347 | 75.325 | 18.883 | −85.153 | 1.00 | 173.29 | D |
| ATOM | 13590 | C | ASP | D | 347 | 76.179 | 15.720 | −86.092 | 1.00 | 162.59 | D |
| ATOM | 13591 | O | ASP | D | 347 | 77.391 | 15.647 | −85.869 | 1.00 | 162.59 | D |
| ATOM | 13592 | N | GLY | D | 348 | 75.257 | 15.332 | −85.214 | 1.00 | 117.12 | D |
| ATOM | 13593 | CA | GLY | D | 348 | 75.638 | 14.773 | −83.929 | 1.00 | 114.43 | D |
| ATOM | 13594 | C | GLY | D | 348 | 75.591 | 13.256 | −83.954 | 1.00 | 111.39 | D |
| ATOM | 13595 | O | GLY | D | 348 | 74.764 | 12.667 | −84.652 | 1.00 | 106.40 | D |
| ATOM | 13596 | N | ASN | D | 349 | 76.473 | 12.619 | −83.188 | 1.00 | 200.84 | D |
| ATOM | 13597 | CA | ASN | D | 349 | 76.524 | 11.164 | −83.151 | 1.00 | 200.84 | D |
| ATOM | 13598 | CB | ASN | D | 349 | 77.495 | 10.686 | −82.059 | 1.00 | 176.83 | D |
| ATOM | 13599 | CG | ASN | D | 349 | 77.488 | 11.574 | −80.831 | 1.00 | 162.01 | D |
| ATOM | 13600 | OD1 | ASN | D | 349 | 77.827 | 12.758 | −80.896 | 1.00 | 162.01 | D |
| ATOM | 13601 | ND2 | ASN | D | 349 | 77.109 | 11.001 | −79.697 | 1.00 | 162.01 | D |
| ATOM | 13602 | C | ASN | D | 349 | 75.159 | 10.488 | −82.996 | 1.00 | 200.84 | D |
| ATOM | 13603 | O | ASN | D | 349 | 74.552 | 10.101 | −83.991 | 1.00 | 200.84 | D |
| ATOM | 13604 | N | MET | D | 350 | 74.664 | 10.362 | −81.769 | 1.00 | 143.27 | D |
| ATOM | 13605 | CA | MET | D | 350 | 73.380 | 9.698 | −81.550 | 1.00 | 139.36 | D |
| ATOM | 13606 | CB | MET | D | 350 | 73.613 | 8.416 | −80.741 | 1.00 | 173.14 | D |
| ATOM | 13607 | CG | MET | D | 350 | 72.358 | 7.624 | −80.425 | 1.00 | 127.82 | D |
| ATOM | 13608 | SD | MET | D | 350 | 72.718 | 6.067 | −79.592 | 1.00 | 127.82 | D |
| ATOM | 13609 | CE | MET | D | 350 | 72.523 | 4.917 | −80.958 | 1.00 | 127.82 | D |
| ATOM | 13610 | C | MET | D | 350 | 72.307 | 10.555 | −80.868 | 1.00 | 136.50 | D |
| ATOM | 13611 | O | MET | D | 350 | 72.617 | 11.517 | −80.169 | 1.00 | 132.93 | D |
| ATOM | 13612 | N | THR | D | 351 | 71.042 | 10.194 | −81.087 | 1.00 | 77.86 | D |
| ATOM | 13613 | CA | THR | D | 351 | 69.904 | 10.896 | −80.489 | 1.00 | 75.82 | D |
| ATOM | 13614 | CB | THR | D | 351 | 69.199 | 11.806 | −81.512 | 1.00 | 81.65 | D |
| ATOM | 13615 | OG1 | THR | D | 351 | 68.669 | 12.941 | −80.837 | 1.00 | 48.77 | D |
| ATOM | 13616 | CG2 | THR | D | 351 | 68.028 | 11.103 | −82.164 | 1.00 | 48.77 | D |
| ATOM | 13617 | C | THR | D | 351 | 68.871 | 9.903 | −79.941 | 1.00 | 70.05 | D |
| ATOM | 13618 | O | THR | D | 351 | 68.506 | 8.934 | −80.611 | 1.00 | 61.64 | D |
| ATOM | 13619 | N | LEU | D | 352 | 68.400 | 10.141 | −78.722 | 1.00 | 73.84 | D |
| ATOM | 13620 | CA | LEU | D | 352 | 67.410 | 9.259 | −78.113 | 1.00 | 76.86 | D |
| ATOM | 13621 | CB | LEU | D | 352 | 67.874 | 8.803 | −76.737 | 1.00 | 95.71 | D |
| ATOM | 13622 | CG | LEU | D | 352 | 68.890 | 7.666 | −76.838 | 1.00 | 76.77 | D |
| ATOM | 13623 | CD1 | LEU | D | 352 | 69.546 | 7.407 | −75.495 | 1.00 | 76.77 | D |
| ATOM | 13624 | CD2 | LEU | D | 352 | 68.183 | 6.424 | −77.337 | 1.00 | 76.77 | D |
| ATOM | 13625 | C | LEU | D | 352 | 66.068 | 9.944 | −78.009 | 1.00 | 73.54 | D |
| ATOM | 13626 | O | LEU | D | 352 | 65.978 | 11.114 | −77.627 | 1.00 | 69.16 | D |
| ATOM | 13627 | N | GLU | D | 353 | 65.022 | 9.207 | −78.357 | 1.00 | 69.89 | D |
| ATOM | 13628 | CA | GLU | D | 353 | 63.673 | 9.750 | −78.337 | 1.00 | 66.14 | D |
| ATOM | 13629 | CB | GLU | D | 353 | 63.170 | 9.932 | −79.767 | 1.00 | 132.82 | D |
| ATOM | 13630 | CG | GLU | D | 353 | 61.788 | 10.520 | −79.861 | 1.00 | 91.37 | D |
| ATOM | 13631 | CD | GLU | D | 353 | 61.303 | 10.583 | −81.283 | 1.00 | 91.37 | D |
| ATOM | 13632 | OE1 | GLU | D | 353 | 61.999 | 11.197 | −82.122 | 1.00 | 91.37 | D |
| ATOM | 13633 | OE2 | GLU | D | 353 | 60.229 | 10.016 | −81.564 | 1.00 | 91.37 | D |
| ATOM | 13634 | C | GLU | D | 353 | 62.715 | 8.852 | −77.566 | 1.00 | 69.39 | D |
| ATOM | 13635 | O | GLU | D | 353 | 62.439 | 7.726 | −77.961 | 1.00 | 70.17 | D |
| ATOM | 13636 | N | CYS | D | 354 | 62.216 | 9.367 | −76.454 | 1.00 | 55.62 | D |
| ATOM | 13637 | CA | CYS | D | 354 | 61.280 | 8.647 | −75.609 | 1.00 | 55.90 | D |
| ATOM | 13638 | C | CYS | D | 354 | 59.899 | 9.150 | −75.989 | 1.00 | 56.99 | D |
| ATOM | 13639 | O | CYS | D | 354 | 59.633 | 10.359 | −75.922 | 1.00 | 55.69 | D |
| ATOM | 13640 | CB | CYS | D | 354 | 61.552 | 8.990 | −74.152 | 1.00 | 108.85 | D |
| ATOM | 13641 | SG | CYS | D | 354 | 60.768 | 7.904 | −72.923 | 1.00 | 86.04 | D |
| ATOM | 13642 | N | THR | D | 355 | 59.021 | 8.251 | −76.414 | 1.00 | 65.31 | D |
| ATOM | 13643 | CA | THR | D | 355 | 57.682 | 8.691 | −76.773 | 1.00 | 54.67 | D |
| ATOM | 13644 | CB | THR | D | 355 | 57.365 | 8.481 | −78.273 | 1.00 | 92.66 | D |
| ATOM | 13645 | OG1 | THR | D | 355 | 56.613 | 7.281 | −78.427 | 1.00 | 41.81 | D |
| ATOM | 13646 | CG2 | THR | D | 355 | 58.626 | 8.364 | −79.091 | 1.00 | 41.81 | D |
| ATOM | 13647 | C | THR | D | 355 | 56.647 | 7.952 | −75.939 | 1.00 | 68.16 | D |
| ATOM | 13648 | O | THR | D | 355 | 56.645 | 6.718 | −75.873 | 1.00 | 60.96 | D |
| ATOM | 13649 | N | VAL | D | 356 | 55.779 | 8.727 | −75.295 | 1.00 | 40.81 | D |
| ATOM | 13650 | CA | VAL | D | 356 | 54.729 | 8.192 | −74.444 | 1.00 | 36.53 | D |
| ATOM | 13651 | CB | VAL | D | 356 | 54.761 | 8.892 | −73.075 | 1.00 | 69.04 | D |
| ATOM | 13652 | CG1 | VAL | D | 356 | 53.872 | 8.173 | −72.095 | 1.00 | 53.57 | D |
| ATOM | 13653 | CG2 | VAL | D | 356 | 56.181 | 8.918 | −72.555 | 1.00 | 53.57 | D |
| ATOM | 13654 | C | VAL | D | 356 | 53.399 | 8.432 | −75.156 | 1.00 | 45.37 | D |
| ATOM | 13655 | O | VAL | D | 356 | 53.030 | 9.570 | −75.448 | 1.00 | 46.48 | D |
| ATOM | 13656 | N | ASN | D | 357 | 52.683 | 7.348 | −75.431 | 1.00 | 54.70 | D |
| ATOM | 13657 | CA | ASN | D | 357 | 51.434 | 7.441 | −76.167 | 1.00 | 50.92 | D |
| ATOM | 13658 | CB | ASN | D | 357 | 51.432 | 6.431 | −77.303 | 1.00 | 119.35 | D |
| ATOM | 13659 | CG | ASN | D | 357 | 52.495 | 6.722 | −78.313 | 1.00 | 73.27 | D |
| ATOM | 13660 | OD1 | ASN | D | 357 | 52.572 | 7.826 | −78.837 | 1.00 | 73.27 | D |
| ATOM | 13661 | ND2 | ASN | D | 357 | 53.334 | 5.741 | −78.590 | 1.00 | 73.27 | D |
| ATOM | 13662 | C | ASN | D | 357 | 50.134 | 7.329 | −75.411 | 1.00 | 55.08 | D |
| ATOM | 13663 | O | ASN | D | 357 | 49.903 | 6.383 | −74.660 | 1.00 | 57.96 | D |
| ATOM | 13664 | N | SER | D | 358 | 49.283 | 8.319 | −75.676 | 1.00 | 55.37 | D |
| ATOM | 13665 | CA | SER | D | 358 | 47.961 | 8.476 | −75.091 | 1.00 | 62.67 | D |
| ATOM | 13666 | CB | SER | D | 358 | 46.909 | 7.819 | −75.988 | 1.00 | 63.02 | D |
| ATOM | 13667 | OG | SER | D | 358 | 47.089 | 6.431 | −76.016 | 1.00 | 61.36 | D |
| ATOM | 13668 | C | SER | D | 358 | 47.800 | 8.009 | −73.646 | 1.00 | 67.39 | D |
| ATOM | 13669 | O | SER | D | 358 | 47.468 | 6.851 | −73.372 | 1.00 | 62.94 | D |
| ATOM | 13670 | N | PHE | D | 359 | 48.033 | 8.955 | −72.737 | 1.00 | 38.74 | D |
| ATOM | 13671 | CA | PHE | D | 359 | 47.919 | 8.754 | −71.284 | 1.00 | 33.05 | D |
| ATOM | 13672 | CB | PHE | D | 359 | 49.292 | 9.003 | −70.629 | 1.00 | 46.86 | D |
| ATOM | 13673 | CG | PHE | D | 359 | 49.989 | 10.253 | −71.115 | 1.00 | 41.46 | D |
| ATOM | 13674 | CD1 | PHE | D | 359 | 49.692 | 11.495 | −70.568 | 1.00 | 41.46 | D |
| ATOM | 13675 | CD2 | PHE | D | 359 | 50.957 | 10.179 | −72.118 | 1.00 | 41.46 | D |
| ATOM | 13676 | CE1 | PHE | D | 359 | 50.351 | 12.641 | −71.008 | 1.00 | 41.46 | D |
| ATOM | 13677 | CE2 | PHE | D | 359 | 51.623 | 11.326 | −72.567 | 1.00 | 41.46 | D |
| ATOM | 13678 | CZ | PHE | D | 359 | 51.321 | 12.554 | −72.010 | 1.00 | 41.46 | D |
| ATOM | 13679 | C | PHE | D | 359 | 46.894 | 9.780 | −70.797 | 1.00 | 39.64 | D |
| ATOM | 13680 | O | PHE | D | 359 | 46.831 | 10.860 | −71.384 | 1.00 | 37.19 | D |
| ATOM | 13681 | N | TYR | D | 360 | 46.092 | 9.499 | −69.765 | 1.00 | 106.28 | D |
| ATOM | 13682 | CA | TYR | D | 360 | 45.155 | 10.548 | −69.409 | 1.00 | 106.06 | D |
| ATOM | 13683 | CB | TYR | D | 360 | 43.849 | 10.091 | −68.763 | 1.00 | 157.25 | D |
| ATOM | 13684 | CG | TYR | D | 360 | 43.115 | 11.348 | −68.287 | 1.00 | 44.39 | D |
| ATOM | 13685 | CD1 | TYR | D | 360 | 42.997 | 12.465 | −69.129 | 1.00 | 44.39 | D |
| ATOM | 13686 | CE1 | TYR | D | 360 | 42.509 | 13.692 | −68.654 | 1.00 | 44.39 | D |
| ATOM | 13687 | CD2 | TYR | D | 360 | 42.699 | 11.493 | −66.956 | 1.00 | 44.39 | D |
| ATOM | 13688 | CE2 | TYR | D | 360 | 42.198 | 12.717 | −66.474 | 1.00 | 44.39 | D |
| ATOM | 13689 | CZ | TYR | D | 360 | 42.111 | 13.812 | −67.328 | 1.00 | 44.39 | D |
| ATOM | 13690 | OH | TYR | D | 360 | 41.639 | 15.029 | −66.867 | 1.00 | 44.39 | D |
| ATOM | 13691 | C | TYR | D | 360 | 45.642 | 11.691 | −68.566 | 1.00 | 107.15 | D |
| ATOM | 13692 | O | TYR | D | 360 | 45.937 | 12.748 | −69.106 | 1.00 | 113.08 | D |
| ATOM | 13693 | N | PRO | D | 361 | 45.702 | 11.525 | −67.237 | 1.00 | 54.84 | D |
| ATOM | 13694 | CD | PRO | D | 361 | 45.877 | 10.301 | −66.439 | 1.00 | 32.45 | D |
| ATOM | 13695 | CA | PRO | D | 361 | 46.175 | 12.732 | −66.537 | 1.00 | 50.43 | D |
| ATOM | 13696 | CB | PRO | D | 361 | 46.491 | 12.228 | −65.126 | 1.00 | 31.69 | D |
| ATOM | 13697 | CG | PRO | D | 361 | 46.798 | 10.777 | −65.330 | 1.00 | 32.45 | D |
| ATOM | 13698 | C | PRO | D | 361 | 47.349 | 13.253 | −67.361 | 1.00 | 57.83 | D |
| ATOM | 13699 | O | PRO | D | 361 | 48.372 | 12.600 | −67.501 | 1.00 | 55.00 | D |
| ATOM | 13700 | N | PRO | D | 362 | 47.160 | 14.421 | −67.991 | 1.00 | 35.69 | D |
| ATOM | 13701 | CD | PRO | D | 362 | 45.976 | 15.277 | −67.770 | 1.00 | 26.51 | D |
| ATOM | 13702 | CA | PRO | D | 362 | 48.138 | 15.094 | −68.857 | 1.00 | 29.40 | D |
| ATOM | 13703 | CB | PRO | D | 362 | 47.381 | 16.347 | −69.322 | 1.00 | 26.51 | D |
| ATOM | 13704 | CG | PRO | D | 362 | 46.477 | 16.637 | −68.182 | 1.00 | 26.51 | D |
| ATOM | 13705 | C | PRO | D | 362 | 49.526 | 15.397 | −68.317 | 1.00 | 32.00 | D |
| ATOM | 13706 | O | PRO | D | 362 | 50.439 | 15.702 | −69.085 | 1.00 | 36.76 | D |
| ATOM | 13707 | N | ASP | D | 363 | 49.685 | 15.315 | −67.001 | 1.00 | 36.69 | D |
| ATOM | 13708 | CA | ASP | D | 363 | 50.984 | 15.581 | −66.399 | 1.00 | 36.54 | D |
| ATOM | 13709 | CB | ASP | D | 363 | 50.847 | 15.992 | −64.913 | 1.00 | 60.57 | D |
| ATOM | 13710 | CG | ASP | D | 363 | 49.899 | 15.091 | −64.117 | 1.00 | 54.78 | D |
| ATOM | 13711 | OD1 | ASP | D | 363 | 48.711 | 14.962 | −64.510 | 1.00 | 54.78 | D |
| ATOM | 13712 | OD2 | ASP | D | 363 | 50.340 | 14.526 | −63.084 | 1.00 | 54.78 | D |
| ATOM | 13713 | C | ASP | D | 363 | 51.879 | 14.364 | −66.549 | 1.00 | 40.08 | D |
| ATOM | 13714 | O | ASP | D | 363 | 51.515 | 13.256 | −66.155 | 1.00 | 36.58 | D |
| ATOM | 13715 | N | VAL | D | 364 | 53.031 | 14.569 | −67.171 | 1.00 | 36.51 | D |
| ATOM | 13716 | CA | VAL | D | 364 | 53.993 | 13.504 | −67.357 | 1.00 | 38.83 | D |
| ATOM | 13717 | CB | VAL | D | 364 | 53.893 | 12.865 | −68.745 | 1.00 | 55.88 | D |
| ATOM | 13718 | CG1 | VAL | D | 364 | 55.154 | 12.144 | −69.064 | 1.00 | 34.27 | D |
| ATOM | 13719 | CG2 | VAL | D | 364 | 52.785 | 11.860 | −68.761 | 1.00 | 34.27 | D |
| ATOM | 13720 | C | VAL | D | 364 | 55.364 | 14.108 | −67.210 | 1.00 | 46.17 | D |
| ATOM | 13721 | O | VAL | D | 364 | 55.601 | 15.236 | −67.647 | 1.00 | 37.91 | D |
| ATOM | 13722 | N | ILE | D | 365 | 56.258 | 13.373 | −66.567 | 1.00 | 51.33 | D |
| ATOM | 13723 | CA | ILE | D | 365 | 57.613 | 13.847 | −66.405 | 1.00 | 45.38 | D |
| ATOM | 13724 | CB | ILE | D | 365 | 57.899 | 14.317 | −64.981 | 1.00 | 49.77 | D |
| ATOM | 13725 | CG2 | ILE | D | 365 | 59.376 | 14.609 | −64.825 | 1.00 | 26.21 | D |
| ATOM | 13726 | CG1 | ILE | D | 365 | 57.091 | 15.590 | −64.705 | 1.00 | 26.21 | D |
| ATOM | 13727 | CD1 | ILE | D | 365 | 57.280 | 16.167 | −63.334 | 1.00 | 26.21 | D |
| ATOM | 13728 | C | ILE | D | 365 | 58.540 | 12.731 | −66.800 | 1.00 | 52.14 | D |
| ATOM | 13729 | O | ILE | D | 365 | 58.260 | 11.549 | −66.590 | 1.00 | 52.91 | D |
| ATOM | 13730 | N | THR | D | 366 | 59.656 | 13.133 | −67.384 | 1.00 | 55.43 | D |
| ATOM | 13731 | CA | THR | D | 366 | 60.628 | 12.202 | −67.893 | 1.00 | 63.71 | D |
| ATOM | 13732 | CB | THR | D | 366 | 60.613 | 12.292 | −69.431 | 1.00 | 59.26 | D |
| ATOM | 13733 | OG1 | THR | D | 366 | 60.314 | 11.008 | −69.972 | 1.00 | 46.94 | D |
| ATOM | 13734 | CG2 | THR | D | 366 | 61.935 | 12.803 | −69.974 | 1.00 | 46.94 | D |
| ATOM | 13735 | C | THR | D | 366 | 62.021 | 12.463 | −67.362 | 1.00 | 72.69 | D |
| ATOM | 13736 | O | THR | D | 366 | 62.465 | 13.604 | −67.298 | 1.00 | 71.93 | D |
| ATOM | 13737 | N | LYS | D | 367 | 62.703 | 11.391 | −66.977 | 1.00 | 49.75 | D |
| ATOM | 13738 | CA | LYS | D | 367 | 64.071 | 11.482 | −66.483 | 1.00 | 52.95 | D |
| ATOM | 13739 | CB | LYS | D | 367 | 64.157 | 11.089 | −65.010 | 1.00 | 56.66 | D |
| ATOM | 13740 | CG | LYS | D | 367 | 63.577 | 12.118 | −64.074 | 1.00 | 47.67 | D |
| ATOM | 13741 | CD | LYS | D | 367 | 63.894 | 11.823 | −62.612 | 1.00 | 47.67 | D |
| ATOM | 13742 | CE | LYS | D | 367 | 63.472 | 12.996 | −61.752 | 1.00 | 47.67 | D |
| ATOM | 13743 | NZ | LYS | D | 367 | 64.043 | 12.910 | −60.391 | 1.00 | 47.67 | D |
| ATOM | 13744 | C | LYS | D | 367 | 64.983 | 10.568 | −67.283 | 1.00 | 55.65 | D |
| ATOM | 13745 | O | LYS | D | 367 | 64.668 | 9.394 | −67.481 | 1.00 | 55.96 | D |
| ATOM | 13746 | N | TRP | D | 368 | 66.101 | 11.119 | −67.755 | 1.00 | 52.65 | D |
| ATOM | 13747 | CA | TRP | D | 368 | 67.092 | 10.345 | −68.500 | 1.00 | 52.71 | D |
| ATOM | 13748 | CB | TRP | D | 368 | 67.593 | 11.128 | −69.710 | 1.00 | 67.37 | D |
| ATOM | 13749 | CG | TRP | D | 368 | 66.555 | 11.263 | −70.766 | 1.00 | 47.29 | D |
| ATOM | 13750 | CD2 | TRP | D | 368 | 66.265 | 10.323 | −71.802 | 1.00 | 47.29 | D |
| ATOM | 13751 | CE2 | TRP | D | 368 | 65.168 | 10.832 | −72.531 | 1.00 | 47.29 | D |
| ATOM | 13752 | CE3 | TRP | D | 368 | 66.822 | 9.097 | −72.184 | 1.00 | 47.29 | D |
| ATOM | 13753 | CD1 | TRP | D | 368 | 65.654 | 12.272 | −70.902 | 1.00 | 47.29 | D |
| ATOM | 13754 | NE1 | TRP | D | 368 | 64.816 | 12.023 | −71.959 | 1.00 | 47.29 | D |
| ATOM | 13755 | CZ2 | TRP | D | 368 | 64.615 | 10.159 | −73.627 | 1.00 | 47.29 | D |
| ATOM | 13756 | CZ3 | TRP | D | 368 | 66.272 | 8.423 | −73.273 | 1.00 | 47.29 | D |
| ATOM | 13757 | CH2 | TRP | D | 368 | 65.179 | 8.959 | −73.983 | 1.00 | 47.29 | D |
| ATOM | 13758 | C | TRP | D | 368 | 68.258 | 10.024 | −67.569 | 1.00 | 54.66 | D |
| ATOM | 13759 | O | TRP | D | 368 | 68.954 | 10.924 | −67.094 | 1.00 | 54.85 | D |
| ATOM | 13760 | N | ILE | D | 369 | 68.461 | 8.737 | −67.307 | 1.00 | 44.25 | D |
| ATOM | 13761 | CA | ILE | D | 369 | 69.519 | 5.287 | −66.411 | 1.00 | 45.62 | D |
| ATOM | 13762 | CB | ILE | D | 369 | 68.902 | 7.668 | −65.162 | 1.00 | 66.79 | D |
| ATOM | 13763 | CG2 | ILE | D | 369 | 69.979 | 7.274 | −64.189 | 1.00 | 44.47 | D |
| ATOM | 13764 | CG1 | ILE | D | 369 | 67.945 | 8.669 | −64.521 | 1.00 | 44.47 | D |
| ATOM | 13765 | CD1 | ILE | D | 369 | 67.166 | 8.100 | −63.374 | 1.00 | 44.47 | D |
| ATOM | 13766 | C | ILE | D | 369 | 70.417 | 7.252 | −67.076 | 1.00 | 47.44 | D |
| ATOM | 13767 | O | ILE | D | 369 | 69.931 | 6.329 | −67.726 | 1.00 | 44.24 | D |
| ATOM | 13768 | N | GLU | D | 370 | 71.728 | 7.407 | −66.922 | 1.00 | 53.66 | D |
| ATOM | 13769 | CA | GLU | D | 370 | 72.655 | 6.454 | −67.512 | 1.00 | 56.31 | D |
| ATOM | 13770 | CB | GLU | D | 370 | 74.090 | 6.885 | −67.227 | 1.00 | 123.61 | D |
| ATOM | 13771 | CG | GLU | D | 370 | 74.519 | 8.032 | −68.119 | 1.00 | 102.25 | D |
| ATOM | 13772 | CD | GLU | D | 370 | 75.824 | 8.653 | −67.698 | 1.00 | 102.25 | D |
| ATOM | 13773 | OE1 | GLU | D | 370 | 76.791 | 7.897 | −67.454 | 1.00 | 102.25 | D |
| ATOM | 13774 | OE2 | GLU | D | 370 | 75.883 | 9.901 | −67.622 | 1.00 | 102.25 | D |
| ATOM | 13775 | C | GLU | D | 370 | 72.353 | 5.067 | −66.949 | 1.00 | 56.49 | D |
| ATOM | 13776 | O | GLU | D | 370 | 72.185 | 4.907 | −65.738 | 1.00 | 55.34 | D |
| ATOM | 13777 | N | SER | D | 371 | 72.265 | 4.071 | −67.829 | 1.00 | 47.76 | D |
| ATOM | 13778 | CA | SER | D | 371 | 71.938 | 2.708 | −67.416 | 1.00 | 48.30 | D |
| ATOM | 13779 | CB | SER | D | 371 | 71.873 | 1.799 | −68.636 | 1.00 | 72.01 | D |
| ATOM | 13780 | OG | SER | D | 371 | 73.108 | 1.804 | −69.317 | 1.00 | 61.09 | D |
| ATOM | 13781 | C | SER | D | 371 | 72.843 | 2.067 | −66.369 | 1.00 | 47.57 | D |
| ATOM | 13782 | O | SER | D | 371 | 72.416 | 1.141 | −65.673 | 1.00 | 48.51 | D |
| ATOM | 13783 | N | GLU | D | 372 | 74.088 | 2.534 | −66.265 | 1.00 | 56.33 | D |
| ATOM | 13784 | CA | GLU | D | 372 | 75.021 | 1.984 | −65.283 | 1.00 | 54.88 | D |
| ATOM | 13785 | CB | GLU | D | 372 | 76.342 | 2.753 | −65.309 | 1.00 | 116.17 | D |
| ATOM | 13786 | CG | GLU | D | 372 | 76.186 | 4.238 | −65.047 | 1.00 | 116.17 | D |
| ATOM | 13787 | CD | GLU | D | 372 | 77.502 | 4.922 | −64.748 | 1.00 | 116.17 | D |
| ATOM | 13788 | OE1 | GLU | D | 372 | 78.133 | 4.560 | −63.735 | 1.00 | 116.17 | D |
| ATOM | 13789 | OE2 | GLU | D | 372 | 77.905 | 5.820 | −65.520 | 1.00 | 116.17 | D |
| ATOM | 13790 | C | GLU | D | 372 | 74.416 | 2.064 | −63.879 | 1.00 | 59.49 | D |
| ATOM | 13791 | O | GLU | D | 372 | 74.554 | 1.141 | −63.074 | 1.00 | 60.56 | D |
| ATOM | 13792 | N | HIS | D | 373 | 73.724 | 3.168 | −63.604 | 1.00 | 73.85 | D |
| ATOM | 13793 | CA | HIS | D | 373 | 73.107 | 3.401 | −62.308 | 1.00 | 77.34 | D |
| ATOM | 13794 | CB | HIS | D | 373 | 72.576 | 4.821 | −62.244 | 1.00 | 85.76 | D |
| ATOM | 13795 | CG | HIS | D | 373 | 73.641 | 5.857 | −62.383 | 1.00 | 55.11 | D |
| ATOM | 13796 | CD2 | HIS | D | 373 | 73.759 | 6.907 | −63.232 | 1.00 | 55.11 | D |
| ATOM | 13797 | ND1 | HIS | D | 373 | 74.756 | 5.890 | −61.573 | 1.00 | 55.11 | D |
| ATOM | 13798 | CE1 | HIS | D | 373 | 75.517 | 6.917 | −61.916 | 1.00 | 55.11 | D |
| ATOM | 13799 | NE2 | HIS | D | 373 | 74.934 | 7.551 | −62.921 | 1.00 | 55.11 | D |
| ATOM | 13800 | C | HIS | D | 373 | 72.005 | 2.431 | −61.925 | 1.00 | 73.11 | D |
| ATOM | 13801 | O | HIS | D | 373 | 71.524 | 2.457 | −60.801 | 1.00 | 68.95 | D |
| ATOM | 13802 | N | PHE | D | 374 | 71.589 | 1.575 | −62.843 | 1.00 | 55.67 | D |
| ATOM | 13803 | CA | PHE | D | 374 | 70.558 | 0.618 | −62.499 | 1.00 | 56.44 | D |
| ATOM | 13804 | CB | PHE | D | 374 | 69.527 | 0.498 | −63.615 | 1.00 | 65.54 | D |
| ATOM | 13805 | CG | PHE | D | 374 | 68.632 | 1.683 | −63.714 | 1.00 | 45.72 | D |
| ATOM | 13806 | CD1 | PHE | D | 374 | 69.045 | 2.827 | −64.384 | 1.00 | 45.72 | D |
| ATOM | 13807 | CD2 | PHE | D | 374 | 67.411 | 1.697 | −63.064 | 1.00 | 45.72 | D |
| ATOM | 13808 | CE1 | PHE | D | 374 | 68.255 | 3.968 | −64.398 | 1.00 | 45.72 | D |
| ATOM | 13809 | CE2 | PHE | D | 374 | 66.619 | 2.832 | −63.073 | 1.00 | 45.72 | D |
| ATOM | 13810 | CZ | PHE | D | 374 | 67.042 | 3.970 | −63.739 | 1.00 | 45.72 | D |
| ATOM | 13811 | C | PHE | D | 374 | 71.146 | −0.744 | −62.176 | 1.00 | 52.52 | D |
| ATOM | 13812 | O | PHE | D | 374 | 70.417 | −1.737 | −62.052 | 1.00 | 50.76 | D |
| ATOM | 13813 | N | LYS | D | 375 | 72.467 | −0.777 | −62.021 | 1.00 | 98.43 | D |
| ATOM | 13814 | CA | LYS | D | 375 | 73.194 | −1.998 | −61.694 | 1.00 | 101.06 | D |
| ATOM | 13815 | CB | LYS | D | 375 | 73.485 | −2.059 | −60.188 | 1.00 | 139.61 | D |
| ATOM | 13816 | CG | LYS | D | 375 | 74.673 | −1.228 | −59.713 | 1.00 | 113.45 | D |
| ATOM | 13817 | CD | LYS | D | 375 | 74.403 | 0.264 | −59.772 | 1.00 | 113.45 | D |
| ATOM | 13818 | CE | LYS | D | 375 | 75.568 | 1.060 | −59.181 | 1.00 | 113.45 | D |
| ATOM | 13819 | NZ | LYS | D | 375 | 75.374 | 2.545 | −59.263 | 1.00 | 113.45 | D |
| ATOM | 13820 | C | LYS | D | 375 | 72.488 | −3.286 | −62.115 | 1.00 | 106.08 | D |
| ATOM | 13821 | O | LYS | D | 375 | 72.256 | −4.166 | −61.289 | 1.00 | 108.34 | D |
| ATOM | 13822 | N | GLY | D | 376 | 72.133 | −3.391 | −63.391 | 1.00 | 82.14 | D |
| ATOM | 13823 | CA | GLY | D | 376 | 71.489 | −4.605 | −63.869 | 1.00 | 82.14 | D |
| ATOM | 13824 | C | GLY | D | 376 | 69.981 | −4.742 | −63.713 | 1.00 | 82.14 | D |
| ATOM | 13825 | O | GLY | D | 376 | 69.334 | −5.418 | −64.519 | 1.00 | 82.14 | D |
| ATOM | 13826 | N | GLU | D | 377 | 69.409 | −4.134 | −62.680 | 1.00 | 88.59 | D |
| ATOM | 13827 | CA | GLU | D | 377 | 67.968 | −4.218 | −62.480 | 1.00 | 88.59 | D |
| ATOM | 13828 | CB | GLU | D | 377 | 67.642 | −4.474 | −61.009 | 1.00 | 126.99 | D |
| ATOM | 13829 | CG | GLU | D | 377 | 66.181 | −4.816 | −60.761 | 1.00 | 126.99 | D |
| ATOM | 13830 | CD | GLU | D | 377 | 65.885 | −5.102 | −59.300 | 1.00 | 126.99 | D |
| ATOM | 13831 | OE1 | GLU | D | 377 | 64.755 | −5.545 | −58.995 | 1.00 | 126.99 | D |
| ATOM | 13832 | OE2 | GLU | D | 377 | 66.782 | −4.880 | −58.457 | 1.00 | 126.99 | D |
| ATOM | 13833 | C | GLU | D | 377 | 67.325 | −2.910 | −62.934 | 1.00 | 88.59 | D |
| ATOM | 13834 | O | GLU | D | 377 | 67.610 | −1.848 | −62.372 | 1.00 | 88.59 | D |
| ATOM | 13835 | N | TYR | D | 378 | 66.457 | −2.992 | −63.944 | 1.00 | 78.89 | D |
| ATOM | 13836 | CA | TYR | D | 378 | 65.795 | −1.808 | −64.478 | 1.00 | 78.89 | D |
| ATOM | 13837 | CB | TYR | D | 378 | 65.622 | −1.943 | −65.990 | 1.00 | 70.52 | D |
| ATOM | 13838 | CG | TYR | D | 378 | 66.941 | −1.874 | −66.715 | 1.00 | 70.52 | D |
| ATOM | 13839 | CD1 | TYR | D | 378 | 67.638 | −3.025 | −67.048 | 1.00 | 70.52 | D |
| ATOM | 13840 | CE1 | TYR | D | 378 | 68.896 | −2.955 | −67.639 | 1.00 | 70.52 | D |
| ATOM | 13841 | CD2 | TYR | D | 378 | 67.530 | −0.648 | −66.995 | 1.00 | 70.52 | D |
| ATOM | 13842 | CE2 | TYR | D | 378 | 68.789 | −0.568 | −67.582 | 1.00 | 70.52 | D |
| ATOM | 13843 | CZ | TYR | D | 378 | 69.467 | −1.724 | −67.897 | 1.00 | 70.52 | D |
| ATOM | 13844 | OH | TYR | D | 378 | 70.725 | −1.635 | −68.441 | 1.00 | 70.52 | D |
| ATOM | 13845 | C | TYR | D | 378 | 64.474 | −1.414 | −63.827 | 1.00 | 78.89 | D |
| ATOM | 13846 | O | TYR | D | 378 | 63.413 | −1.442 | −64.453 | 1.00 | 78.89 | D |
| ATOM | 13847 | N | LYS | D | 379 | 64.573 | −1.041 | −62.555 | 1.00 | 56.93 | D |
| ATOM | 13848 | CA | LYS | D | 379 | 63.452 | −0.585 | −61.747 | 1.00 | 56.93 | D |
| ATOM | 13849 | CB | LYS | D | 379 | 63.054 | −1.634 | −60.710 | 1.00 | 91.14 | D |
| ATOM | 13850 | CG | LYS | D | 379 | 62.465 | −2.901 | −61.285 | 1.00 | 91.14 | D |
| ATOM | 13851 | CD | LYS | D | 379 | 62.128 | −3.876 | −60.170 | 1.00 | 91.14 | D |
| ATOM | 13852 | CE | LYS | D | 379 | 61.705 | −5.240 | −60.700 | 1.00 | 91.14 | D |
| ATOM | 13853 | NZ | LYS | D | 379 | 61.557 | −6.228 | −59.583 | 1.00 | 91.14 | D |
| ATOM | 13854 | C | LYS | D | 379 | 63.950 | 0.681 | −61.038 | 1.00 | 56.93 | D |
| ATOM | 13855 | O | LYS | D | 379 | 65.090 | 0.740 | −60.573 | 1.00 | 56.93 | D |
| ATOM | 13856 | N | TYR | D | 380 | 63.102 | 1.703 | −60.983 | 1.00 | 52.33 | D |
| ATOM | 13857 | CA | TYR | D | 380 | 63.453 | 2.956 | −60.324 | 1.00 | 52.33 | D |
| ATOM | 13858 | CB | TYR | D | 380 | 62.681 | 4.099 | −60.961 | 1.00 | 40.36 | D |
| ATOM | 13859 | CG | TYR | D | 380 | 63.054 | 5.448 | −60.426 | 1.00 | 40.36 | D |
| ATOM | 13860 | CD1 | TYR | D | 380 | 64.119 | 6.165 | −60.976 | 1.00 | 40.36 | D |
| ATOM | 13861 | CE1 | TYR | D | 380 | 64.448 | 7.429 | −60.508 | 1.00 | 40.36 | D |
| ATOM | 13862 | CD2 | TYR | D | 380 | 62.328 | 6.028 | −59.380 | 1.00 | 40.36 | D |
| ATOM | 13863 | CE2 | TYR | D | 380 | 62.647 | 7.298 | −58.900 | 1.00 | 40.36 | D |
| ATOM | 13864 | CZ | TYR | D | 380 | 63.705 | 7.994 | −59.472 | 1.00 | 40.36 | D |
| ATOM | 13865 | OH | TYR | D | 380 | 63.987 | 9.266 | −59.033 | 1.00 | 40.36 | D |
| ATOM | 13866 | C | TYR | D | 380 | 63.054 | 2.829 | −58.862 | 1.00 | 52.33 | D |
| ATOM | 13867 | O | TYR | D | 380 | 61.871 | 2.783 | −58.542 | 1.00 | 52.33 | D |
| ATOM | 13868 | N | VAL | D | 381 | 64.035 | 2.766 | −57.976 | 1.00 | 45.05 | D |
| ATOM | 13869 | CA | VAL | D | 381 | 63.754 | 2.617 | −56.556 | 1.00 | 45.05 | D |
| ATOM | 13870 | CB | VAL | D | 381 | 64.959 | 1.995 | −55.823 | 1.00 | 25.93 | D |
| ATOM | 13871 | CG1 | VAL | D | 381 | 64.620 | 1.760 | −54.358 | 1.00 | 25.93 | D |
| ATOM | 13872 | CG2 | VAL | D | 381 | 65.326 | 0.687 | −56.504 | 1.00 | 25.93 | D |
| ATOM | 13873 | C | VAL | D | 381 | 63.407 | 3.963 | −55.928 | 1.00 | 45.05 | D |
| ATOM | 13874 | O | VAL | D | 381 | 64.212 | 4.895 | −55.949 | 1.00 | 45.05 | D |
| ATOM | 13875 | N | ASN | D | 382 | 62.214 | 4.046 | −55.348 | 1.00 | 70.31 | D |
| ATOM | 13876 | CA | ASN | D | 382 | 61.734 | 5.285 | −54.756 | 1.00 | 70.31 | D |
| ATOM | 13877 | CB | ASN | D | 382 | 60.613 | 5.013 | −53.775 | 1.00 | 40.43 | D |
| ATOM | 13878 | CG | ASN | D | 382 | 59.714 | 6.221 | −53.567 | 1.00 | 40.43 | D |
| ATOM | 13879 | OD1 | ASN | D | 382 | 60.146 | 7.372 | −53.659 | 1.00 | 40.43 | D |
| ATOM | 13880 | ND2 | ASN | D | 302 | 58.448 | 5.952 | −53.264 | 1.00 | 40.43 | D |
| ATOM | 13881 | C | ASN | D | 382 | 62.756 | 6.168 | −54.064 | 1.00 | 70.31 | D |
| ATOM | 13882 | O | ASN | D | 382 | 63.223 | 7.145 | −54.644 | 1.00 | 70.31 | D |
| ATOM | 13883 | N | GLY | D | 383 | 63.116 | 5.859 | −52.826 | 1.00 | 32.01 | D |
| ATOM | 13884 | CA | GLY | D | 383 | 64.059 | 6.752 | −52.162 | 1.00 | 32.01 | D |
| ATOM | 13885 | C | GLY | D | 383 | 65.515 | 6.774 | −52.633 | 1.00 | 32.01 | D |
| ATOM | 13886 | O | GLY | D | 383 | 66.331 | 7.493 | −52.078 | 1.00 | 32.01 | D |
| ATOM | 13887 | N | ARG | D | 384 | 65.856 | 5.993 | −53.651 | 1.00 | 53.61 | D |
| ATOM | 13888 | CA | ARG | D | 384 | 67.239 | 5.938 | −54.106 | 1.00 | 53.61 | D |
| ATOM | 13889 | CB | ARG | D | 384 | 67.446 | 4.699 | −54.987 | 1.00 | 54.60 | D |
| ATOM | 13890 | CG | ARG | D | 384 | 68.902 | 4.409 | −55.360 | 1.00 | 54.60 | D |
| ATOM | 13891 | CD | ARG | D | 384 | 69.025 | 2.987 | −55.897 | 1.00 | 54.60 | D |
| ATOM | 13892 | NE | ARG | D | 384 | 68.612 | 2.023 | −54.885 | 1.00 | 54.60 | D |
| ATOM | 13893 | CZ | ARG | D | 384 | 68.432 | 0.733 | −55.110 | 1.00 | 54.60 | D |
| ATOM | 13894 | NH1 | ARG | D | 384 | 68.626 | 0.235 | −56.320 | 1.00 | 54.60 | D |
| ATOM | 13895 | NH2 | ARG | D | 384 | 68.058 | −0.055 | −54.118 | 1.00 | 54.60 | D |
| ATOM | 13896 | C | ARG | D | 384 | 67.681 | 7.180 | −54.848 | 1.00 | 53.61 | D |
| ATOM | 13897 | O | ARG | D | 384 | 66.887 | 7.842 | −55.508 | 1.00 | 53.61 | D |
| ATOM | 13898 | N | TYR | D | 385 | 68.961 | 7.494 | −54.733 | 1.00 | 52.59 | D |
| ATOM | 13899 | CA | TYR | D | 385 | 69.532 | 8.640 | −55.420 | 1.00 | 52.59 | D |
| ATOM | 13900 | CB | TYR | D | 385 | 70.745 | 9.142 | −54.664 | 1.00 | 61.53 | D |
| ATOM | 13901 | CG | TYR | D | 385 | 71.484 | 10.201 | −55.423 | 1.00 | 61.53 | D |
| ATOM | 13902 | CD1 | TYR | D | 385 | 70.995 | 11.503 | −55.495 | 1.00 | 61.53 | D |
| ATOM | 13903 | CE1 | TYR | D | 385 | 71.628 | 12.474 | −56.271 | 1.00 | 61.53 | D |
| ATOM | 13904 | CD2 | TYR | D | 385 | 72.632 | 9.893 | −56.142 | 1.00 | 61.53 | D |
| ATOM | 13905 | CE2 | TYR | D | 385 | 73.274 | 10.855 | −56.924 | 1.00 | 61.53 | D |
| ATOM | 13906 | CZ | TYR | D | 385 | 72.762 | 12.142 | −56.982 | 1.00 | 61.53 | D |
| ATOM | 13907 | OH | TYR | D | 385 | 73.367 | 13.100 | −57.759 | 1.00 | 61.53 | D |
| ATOM | 13908 | C | TYR | D | 385 | 69.968 | 8.272 | −56.847 | 1.00 | 52.59 | D |
| ATOM | 13909 | O | TYR | D | 385 | 70.493 | 7.180 | −57.080 | 1.00 | 52.59 | D |
| ATOM | 13910 | N | TYR | D | 386 | 69.759 | 9.183 | −57.797 | 1.00 | 48.89 | D |
| ATOM | 13911 | CA | TYR | D | 386 | 70.147 | 8.947 | −59.188 | 1.00 | 48.89 | D |
| ATOM | 13912 | CB | TYR | D | 386 | 68.974 | 8.421 | −60.025 | 1.00 | 43.90 | D |
| ATOM | 13913 | CG | TYR | D | 386 | 68.409 | 7.075 | −59.647 | 1.00 | 43.90 | D |
| ATOM | 13914 | CD1 | TYR | D | 386 | 67.298 | 6.982 | −58.834 | 1.00 | 43.90 | D |
| ATOM | 13915 | CE1 | TYR | D | 386 | 66.733 | 5.752 | −58.524 | 1.00 | 43.90 | D |
| ATOM | 13916 | CD2 | TYR | D | 386 | 68.959 | 5.894 | −60.147 | 1.00 | 43.90 | D |
| ATOM | 13917 | CE2 | TYR | D | 386 | 68.402 | 4.657 | −59.839 | 1.00 | 43.90 | D |
| ATOM | 13918 | CZ | TYR | D | 386 | 67.286 | 4.601 | −59.023 | 1.00 | 43.90 | D |
| ATOM | 13919 | OH | TYR | D | 386 | 66.719 | 3.401 | −58.688 | 1.00 | 43.90 | D |
| ATOM | 13920 | C | TYR | D | 386 | 70.632 | 10.211 | −59.889 | 1.00 | 48.89 | D |
| ATOM | 13921 | O | TYR | D | 386 | 69.930 | 11.208 | −59.912 | 1.00 | 48.89 | D |
| ATOM | 13922 | N | PRO | D | 387 | 71.850 | 10.200 | −60.452 | 1.00 | 58.54 | D |
| ATOM | 13923 | CD | PRO | D | 387 | 72.934 | 9.220 | −60.287 | 1.00 | 97.17 | D |
| ATOM | 13924 | CA | PRO | D | 387 | 72.327 | 11.400 | −61.158 | 1.00 | 58.54 | D |
| ATOM | 13925 | CB | PRO | D | 387 | 73.743 | 11.015 | −61.555 | 1.00 | 97.17 | D |
| ATOM | 13926 | CG | PRO | D | 387 | 74.156 | 10.081 | −60.456 | 1.00 | 97.17 | D |
| ATOM | 13927 | C | PRO | D | 387 | 71.383 | 11.427 | −62.359 | 1.00 | 58.54 | D |
| ATOM | 13928 | O | PRO | D | 387 | 71.021 | 10.353 | −62.852 | 1.00 | 58.54 | D |
| ATOM | 13929 | N | GLU | D | 388 | 70.980 | 12.587 | −62.870 | 1.00 | 85.17 | D |
| ATOM | 13930 | CA | GLU | D | 388 | 70.014 | 12.497 | −63.955 | 1.00 | 85.17 | D |
| ATOM | 13931 | CB | GLU | D | 388 | 68.611 | 12.674 | −63.372 | 1.00 | 100.41 | D |
| ATOM | 13932 | CG | GLU | D | 388 | 68.313 | 14.072 | −62.869 | 1.00 | 100.41 | D |
| ATOM | 13933 | CD | GLU | D | 388 | 66.844 | 14.271 | −62.563 | 1.00 | 100.41 | D |
| ATOM | 13934 | OE1 | GLU | D | 388 | 66.023 | 14.090 | −63.487 | 1.00 | 100.41 | D |
| ATOM | 13935 | OE2 | GLU | D | 388 | 66.509 | 14.608 | −61.407 | 1.00 | 100.41 | D |
| ATOM | 13936 | C | GLU | D | 388 | 70.049 | 13.251 | −65.277 | 1.00 | 85.17 | D |
| ATOM | 13937 | O | GLU | D | 388 | 69.009 | 13.340 | −65.926 | 1.00 | 85.17 | D |
| ATOM | 13938 | N | TRP | D | 389 | 71.180 | 13.783 | −65.720 | 1.00 | 70.99 | D |
| ATOM | 13939 | CA | TRP | D | 389 | 71.159 | 14.470 | −67.021 | 1.00 | 70.99 | D |
| ATOM | 13940 | CB | TRP | D | 389 | 70.905 | 13.463 | −68.147 | 1.00 | 110.99 | D |
| ATOM | 13941 | CG | TRP | D | 389 | 72.115 | 12.964 | −68.861 | 1.00 | 110.99 | D |
| ATOM | 13942 | CD2 | TRP | D | 389 | 73.466 | 13.436 | −68.728 | 1.00 | 110.99 | D |
| ATOM | 13943 | CE2 | TRP | D | 389 | 74.257 | 12.689 | −69.629 | 1.00 | 110.99 | D |
| ATOM | 13944 | CE3 | TRP | D | 389 | 74.086 | 14.420 | −67.936 | 1.00 | 110.99 | D |
| ATOM | 13945 | CD1 | TRP | D | 389 | 72.146 | 11.982 | −69.806 | 1.00 | 110.99 | D |
| ATOM | 13946 | NE1 | TRP | D | 389 | 73.425 | 11.810 | −70.272 | 1.00 | 110.99 | D |
| ATOM | 13947 | CZ2 | TRP | D | 389 | 75.638 | 12.893 | −69.763 | 1.00 | 110.99 | D |
| ATOM | 13948 | CZ3 | TRP | D | 389 | 75.459 | 14.621 | −68.069 | 1.00 | 110.99 | D |
| ATOM | 13949 | CH2 | TRP | D | 389 | 76.218 | 13.859 | −68.978 | 1.00 | 110.99 | D |
| ATOM | 13950 | C | TRP | D | 389 | 70.065 | 15.543 | −67.105 | 1.00 | 70.99 | D |
| ATOM | 13951 | O | TRP | D | 389 | 69.077 | 15.366 | −67.830 | 1.00 | 70.99 | D |
| ATOM | 13952 | N | GLY | D | 390 | 70.240 | 16.648 | −66.382 | 1.00 | 81.35 | D |
| ATOM | 13953 | CA | GLY | D | 390 | 69.236 | 17.698 | −66.399 | 1.00 | 81.35 | D |
| ATOM | 13954 | C | GLY | D | 390 | 69.015 | 18.346 | −67.756 | 1.00 | 81.35 | D |
| ATOM | 13955 | O | GLY | D | 390 | 69.900 | 18.349 | −68.606 | 1.00 | 81.35 | D |
| ATOM | 13956 | N | ARG | D | 391 | 67.818 | 18.886 | −67.963 | 1.00 | 91.82 | D |
| ATOM | 13957 | CA | ARG | D | 391 | 67.478 | 19.574 | −69.205 | 1.00 | 91.82 | D |
| ATOM | 13958 | CB | ARG | D | 391 | 65.986 | 19.402 | −69.511 | 1.00 | 94.74 | D |
| ATOM | 13959 | CG | ARG | D | 391 | 65.540 | 17.945 | −69.647 | 1.00 | 94.74 | D |
| ATOM | 13960 | CD | ARG | D | 391 | 65.497 | 17.467 | −71.110 | 1.00 | 94.74 | D |
| ATOM | 13961 | NE | ARG | D | 391 | 64.188 | 17.678 | −71.741 | 1.00 | 94.74 | D |
| ATOM | 13962 | CZ | ARG | D | 391 | 63.735 | 18.848 | −72.191 | 1.00 | 94.74 | D |
| ATOM | 13963 | NH1 | ARG | D | 391 | 64.483 | 19.942 | −72.095 | 1.00 | 94.74 | D |
| ATOM | 13964 | NH2 | ARG | D | 391 | 62.524 | 18.927 | −72.731 | 1.00 | 94.74 | D |
| ATOM | 13965 | C | ARG | D | 391 | 67.805 | 21.051 | −68.972 | 1.00 | 91.82 | D |
| ATOM | 13966 | O | ARG | D | 391 | 68.040 | 21.462 | −67.839 | 1.00 | 91.82 | D |
| ATOM | 13967 | N | LYS | D | 392 | 67.832 | 21.848 | −70.032 | 1.00 | 61.53 | D |
| ATOM | 13968 | CA | LYS | D | 392 | 68.142 | 23.270 | −69.886 | 1.00 | 61.53 | D |
| ATOM | 13969 | CB | LYS | D | 392 | 69.275 | 23.670 | −70.834 | 1.00 | 98.51 | D |
| ATOM | 13970 | CG | LYS | D | 392 | 69.655 | 25.143 | −70.740 | 1.00 | 98.51 | D |
| ATOM | 13971 | CD | LYS | D | 392 | 70.606 | 25.556 | −71.851 | 1.00 | 98.51 | D |
| ATOM | 13972 | CE | LYS | D | 392 | 70.836 | 27.058 | −71.838 | 1.00 | 98.51 | D |
| ATOM | 13973 | NZ | LYS | D | 392 | 71.619 | 27.508 | −73.024 | 1.00 | 98.51 | D |
| ATOM | 13974 | C | LYS | D | 392 | 66.914 | 24.142 | −70.165 | 1.00 | 61.53 | D |
| ATOM | 13975 | O | LYS | D | 392 | 66.406 | 24.169 | −71.284 | 1.00 | 61.53 | D |
| ATOM | 13976 | N | SER | D | 393 | 66.449 | 24.855 | −69.142 | 1.00 | 55.24 | D |
| ATOM | 13977 | CA | SER | D | 393 | 65.277 | 25.722 | −69.263 | 1.00 | 55.24 | D |
| ATOM | 13978 | CB | SER | D | 393 | 64.000 | 24.889 | −69.292 | 1.00 | 61.30 | D |
| ATOM | 13979 | OG | SER | D | 393 | 62.856 | 25.718 | −69.288 | 1.00 | 61.30 | D |
| ATOM | 13980 | C | SER | D | 393 | 65.212 | 26.666 | −68.081 | 1.00 | 55.24 | D |
| ATOM | 13981 | O | SER | D | 393 | 65.785 | 26.383 | −67.032 | 1.00 | 55.24 | D |
| ATOM | 13982 | N | ASN | D | 394 | 64.521 | 27.790 | −68.239 | 1.00 | 62.99 | D |
| ATOM | 13983 | CA | ASN | D | 394 | 64.409 | 28.734 | −67.135 | 1.00 | 62.99 | D |
| ATOM | 13984 | CB | ASN | D | 394 | 63.955 | 30.094 | −67.631 | 1.00 | 62.50 | D |
| ATOM | 13985 | CG | ASN | D | 394 | 65.024 | 30.804 | −68.412 | 1.00 | 62.50 | D |
| ATOM | 13986 | OD1 | ASN | D | 394 | 66.168 | 30.914 | −67.962 | 1.00 | 62.50 | D |
| ATOM | 13987 | ND2 | ASN | D | 394 | 64.659 | 31.303 | −69.594 | 1.00 | 62.50 | D |
| ATOM | 13988 | C | ASN | D | 394 | 63.419 | 28.203 | −66.116 | 1.00 | 62.99 | D |
| ATOM | 13989 | O | ASN | D | 394 | 63.390 | 28.634 | −64.964 | 1.00 | 62.99 | D |
| ATOM | 13990 | N | TYR | D | 395 | 62.609 | 27.254 | −66.556 | 1.00 | 56.45 | D |
| ATOM | 13991 | CA | TYR | D | 395 | 61.621 | 26.644 | −65.698 | 1.00 | 56.45 | D |
| ATOM | 13992 | CB | TYR | D | 395 | 60.343 | 26.387 | −66.486 | 1.00 | 44.84 | D |
| ATOM | 13993 | CG | TYR | D | 395 | 59.657 | 27.628 | −66.978 | 1.00 | 44.84 | D |
| ATOM | 13994 | CD1 | TYR | D | 395 | 59.878 | 28.106 | −68.264 | 1.00 | 44.84 | D |
| ATOM | 13995 | CE1 | TYR | D | 395 | 59.240 | 29.256 | −68.728 | 1.00 | 44.84 | D |
| ATOM | 13996 | CD2 | TYR | D | 395 | 58.777 | 28.333 | −66.153 | 1.00 | 44.84 | D |
| ATOM | 13997 | CE2 | TYR | D | 395 | 58.136 | 29.481 | −66.601 | 1.00 | 44.84 | D |
| ATOM | 13998 | CZ | TYR | D | 395 | 58.374 | 29.940 | −67.897 | 1.00 | 44.84 | D |
| ATOM | 13999 | OH | TYR | D | 395 | 57.749 | 31.088 | −68.355 | 1.00 | 44.84 | D |
| ATOM | 14000 | C | TYR | D | 395 | 62.163 | 25.320 | −65.185 | 1.00 | 56.45 | D |
| ATOM | 14001 | O | TYR | D | 395 | 63.025 | 24.719 | −65.822 | 1.00 | 56.45 | D |
| ATOM | 14002 | N | GLU | D | 396 | 61.653 | 24.873 | −64.038 | 1.00 | 42.07 | D |
| ATOM | 14003 | CA | GLU | D | 396 | 62.054 | 23.600 | −63.444 | 1.00 | 42.07 | D |
| ATOM | 14004 | CB | GLU | D | 396 | 62.204 | 23.740 | −61.932 | 1.00 | 119.94 | D |
| ATOM | 14005 | CG | GLU | D | 396 | 63.021 | 24.930 | −61.487 | 1.00 | 119.94 | D |
| ATOM | 14006 | CD | GLU | D | 396 | 62.916 | 25.160 | −59.993 | 1.00 | 119.94 | D |
| ATOM | 14007 | OE1 | GLU | D | 396 | 61.774 | 25.265 | −59.491 | 1.00 | 119.94 | D |
| ATOM | 14008 | OE2 | GLU | D | 396 | 63.968 | 25.240 | −59.323 | 1.00 | 119.94 | D |
| ATOM | 14009 | C | GLU | D | 396 | 60.921 | 22.618 | −63.740 | 1.00 | 42.07 | D |
| ATOM | 14010 | O | GLU | D | 396 | 59.747 | 22.984 | −63.657 | 1.00 | 42.07 | D |
| ATOM | 14011 | N | PRO | D | 397 | 61.247 | 21.366 | −64.103 | 1.00 | 51.48 | D |
| ATOM | 14012 | CD | PRO | D | 397 | 62.555 | 20.695 | −64.148 | 1.00 | 32.62 | D |
| ATOM | 14013 | CA | PRO | D | 397 | 60.166 | 20.414 | −64.381 | 1.00 | 51.48 | D |
| ATOM | 14014 | CB | PRO | D | 397 | 60.907 | 19.091 | −64.547 | 1.00 | 32.62 | D |
| ATOM | 14015 | CG | PRO | D | 397 | 62.182 | 19.303 | −63.782 | 1.00 | 32.62 | D |
| ATOM | 14016 | C | PRO | D | 397 | 59.162 | 20.405 | −63.231 | 1.00 | 51.48 | D |
| ATOM | 14017 | O | PRO | D | 397 | 59.540 | 20.376 | −62.053 | 1.00 | 51.48 | D |
| ATOM | 14018 | N | GLY | D | 398 | 57.884 | 20.439 | −63.575 | 1.00 | 46.65 | D |
| ATOM | 14019 | CA | GLY | D | 398 | 56.852 | 20.495 | −62.556 | 1.00 | 46.65 | D |
| ATOM | 14020 | C | GLY | D | 398 | 56.090 | 21.806 | −62.728 | 1.00 | 46.65 | D |
| ATOM | 14021 | O | GLY | D | 398 | 54.859 | 21.842 | −62.599 | 1.00 | 46.65 | D |
| ATOM | 14022 | N | GLU | D | 399 | 56.820 | 22.886 | −63.020 | 1.00 | 36.01 | D |
| ATOM | 14023 | CA | GLU | D | 399 | 56.198 | 24.187 | −63.240 | 1.00 | 36.01 | D |
| ATOM | 14024 | CB | GLU | D | 399 | 57.253 | 25.296 | −63.263 | 1.00 | 51.98 | D |
| ATOM | 14025 | CG | GLU | D | 399 | 58.146 | 25.329 | −62.028 | 1.00 | 51.98 | D |
| ATOM | 14026 | CD | GLU | D | 399 | 59.163 | 26.485 | −62.024 | 1.00 | 51.98 | D |
| ATOM | 14027 | OE1 | GLU | D | 399 | 59.781 | 26.747 | −63.078 | 1.00 | 51.98 | D |
| ATOM | 14028 | OE2 | GLU | D | 399 | 59.359 | 27.118 | −60.959 | 1.00 | 51.98 | D |
| ATOM | 14029 | C | GLU | D | 399 | 55.509 | 24.101 | −64.601 | 1.00 | 36.01 | D |
| ATOM | 14030 | O | GLU | D | 399 | 56.073 | 23.536 | −65.541 | 1.00 | 36.01 | D |
| ATOM | 14031 | N | PRO | D | 400 | 54.275 | 24.632 | −64.730 | 1.00 | 57.31 | D |
| ATOM | 14032 | CD | PRO | D | 400 | 53.376 | 25.243 | −63.744 | 1.00 | 39.59 | D |
| ATOM | 14033 | CA | PRO | D | 400 | 53.625 | 24.541 | −66.043 | 1.00 | 52.39 | D |
| ATOM | 14034 | CB | PRO | D | 400 | 52.275 | 25.222 | −65.822 | 1.00 | 88.20 | D |
| ATOM | 14035 | CG | PRO | D | 400 | 52.489 | 26.061 | −64.630 | 1.00 | 39.59 | D |
| ATOM | 14036 | C | PRO | D | 400 | 54.432 | 25.150 | −67.191 | 1.00 | 43.14 | D |
| ATOM | 14037 | O | PRO | D | 400 | 54.225 | 24.817 | −68.357 | 1.00 | 43.35 | D |
| ATOM | 14038 | N | GLY | D | 401 | 55.379 | 26.016 | −66.861 | 1.00 | 60.13 | D |
| ATOM | 14039 | CA | GLY | D | 401 | 56.195 | 26.624 | −67.894 | 1.00 | 59.69 | D |
| ATOM | 14040 | C | GLY | D | 401 | 57.245 | 25.712 | −68.518 | 1.00 | 59.48 | D |
| ATOM | 14041 | O | GLY | D | 401 | 57.842 | 26.067 | −69.527 | 1.00 | 60.19 | D |
| ATOM | 14042 | N | PHE | D | 402 | 57.490 | 24.544 | −67.937 | 1.00 | 51.79 | D |
| ATOM | 14043 | CA | PHE | D | 402 | 58.488 | 23.637 | −68.494 | 1.00 | 51.79 | D |
| ATOM | 14044 | CB | PHE | D | 402 | 58.736 | 22.470 | −67.555 | 1.00 | 159.30 | D |
| ATOM | 14045 | CG | PHE | D | 402 | 60.041 | 21.804 | −67.793 | 1.00 | 57.63 | D |
| ATOM | 14046 | CD1 | PHE | D | 402 | 61.221 | 22.408 | −67.370 | 1.00 | 57.63 | D |
| ATOM | 14047 | CD2 | PHE | D | 402 | 60.112 | 20.602 | −68.498 | 1.00 | 57.63 | D |
| ATOM | 14048 | CE1 | PHE | D | 402 | 62.460 | 21.831 | −67.647 | 1.00 | 57.63 | D |
| ATOM | 14049 | CE2 | PHE | D | 402 | 61.344 | 20.012 | −68.781 | 1.00 | 57.63 | D |
| ATOM | 14050 | CZ | PHE | D | 402 | 62.523 | 20.631 | −68.354 | 1.00 | 57.63 | D |
| ATOM | 14051 | C | PHE | D | 402 | 58.049 | 23.091 | −69.858 | 1.00 | 51.79 | D |
| ATOM | 14052 | O | PHE | D | 402 | 56.942 | 22.562 | −69.997 | 1.00 | 51.79 | D |
| ATOM | 14053 | N | PRO | D | 403 | 58.926 | 23.191 | −70.877 | 1.00 | 68.31 | D |
| ATOM | 14054 | CD | PRO | D | 403 | 60.269 | 23.771 | −70.771 | 1.00 | 56.31 | D |
| ATOM | 14055 | CA | PRO | D | 403 | 58.684 | 22.735 | −72.251 | 1.00 | 68.31 | D |
| ATOM | 14056 | CB | PRO | D | 403 | 59.883 | 23.285 | −73.025 | 1.00 | 161.14 | D |
| ATOM | 14057 | CG | PRO | D | 403 | 60.452 | 24.329 | −72.138 | 1.00 | 56.31 | D |
| ATOM | 14058 | C | PRO | D | 403 | 58.596 | 21.224 | −72.399 | 1.00 | 68.31 | D |
| ATOM | 14059 | O | PRO | D | 403 | 59.614 | 20.541 | −72.382 | 1.00 | 68.31 | D |
| ATOM | 14060 | N | TRP | D | 404 | 57.385 | 20.699 | −72.522 | 1.00 | 60.52 | D |
| ATOM | 14061 | CA | TRP | D | 404 | 57.198 | 19.272 | −72.737 | 1.00 | 60.52 | D |
| ATOM | 14062 | CB | TRP | D | 404 | 56.346 | 18.636 | −71.640 | 1.00 | 54.78 | D |
| ATOM | 14063 | CG | TRP | D | 404 | 57.034 | 18.441 | −70.337 | 1.00 | 48.02 | D |
| ATOM | 14064 | CD2 | TRP | D | 404 | 58.173 | 17.614 | −70.074 | 1.00 | 48.02 | D |
| ATOM | 14065 | CE2 | TRP | D | 404 | 58.442 | 17.699 | −68.690 | 1.00 | 48.02 | D |
| ATOM | 14066 | CE3 | TRP | D | 404 | 58.994 | 16.807 | −70.871 | 1.00 | 48.02 | D |
| ATOM | 14067 | CD1 | TRP | D | 404 | 56.675 | 18.978 | −69.145 | 1.00 | 48.02 | D |
| ATOM | 14068 | NE1 | TRP | D | 404 | 57.511 | 18.540 | −68.148 | 1.00 | 48.02 | D |
| ATOM | 14069 | CZ2 | TRP | D | 404 | 59.498 | 17.006 | −68.082 | 1.00 | 48.02 | D |
| ATOM | 14070 | CZ3 | TRP | D | 404 | 60.050 | 16.116 | −70.266 | 1.00 | 48.02 | D |
| ATOM | 14071 | CH2 | TRP | D | 404 | 60.288 | 16.222 | −68.883 | 1.00 | 48.02 | D |
| ATOM | 14072 | C | TRP | D | 404 | 56.451 | 19.191 | −74.061 | 1.00 | 60.52 | D |
| ATOM | 14073 | O | TRP | D | 404 | 55.456 | 19.892 | −74.261 | 1.00 | 60.52 | D |
| ATOM | 14074 | N | ASN | D | 405 | 56.931 | 18.353 | −74.971 | 1.00 | 59.38 | D |
| ATOM | 14075 | CA | ASN | D | 405 | 56.280 | 18.214 | −76.262 | 1.00 | 59.41 | D |
| ATOM | 14076 | CB | ASN | D | 405 | 57.250 | 17.611 | −77.271 | 1.00 | 91.50 | D |
| ATOM | 14077 | CG | ASN | D | 405 | 56.858 | 17.913 | −78.690 | 1.00 | 91.50 | D |
| ATOM | 14078 | OD1 | ASN | D | 405 | 55.874 | 17.381 | −79.207 | 1.00 | 91.50 | D |
| ATOM | 14079 | ND2 | ASN | D | 405 | 57.619 | 18.791 | −79.331 | 1.00 | 91.50 | D |
| ATOM | 14080 | C | ASN | D | 405 | 55.032 | 17.341 | −76.132 | 1.00 | 68.35 | D |
| ATOM | 14081 | O | ASN | D | 405 | 55.016 | 16.174 | −76.531 | 1.00 | 61.41 | D |
| ATOM | 14082 | N | ILE | D | 406 | 53.985 | 17.918 | −75.559 | 1.00 | 62.97 | D |
| ATOM | 14083 | CA | ILE | D | 406 | 52.733 | 17.204 | −75.370 | 1.00 | 55.17 | D |
| ATOM | 14084 | CB | ILE | D | 406 | 52.242 | 17.317 | −73.909 | 1.00 | 63.64 | D |
| ATOM | 14085 | CG2 | ILE | D | 406 | 50.995 | 16.465 | −73.700 | 1.00 | 51.26 | D |
| ATOM | 14086 | CG1 | ILE | D | 406 | 53.356 | 16.867 | −72.962 | 1.00 | 51.26 | D |
| ATOM | 14087 | CD1 | ILE | D | 406 | 52.957 | 16.862 | −71.499 | 1.00 | 51.26 | D |
| ATOM | 14088 | C | ILE | D | 406 | 51.669 | 17.782 | −76.285 | 1.00 | 62.04 | D |
| ATOM | 14089 | O | ILE | D | 406 | 51.623 | 18.985 | −76.511 | 1.00 | 57.14 | D |
| ATOM | 14090 | N | LYS | D | 407 | 50.827 | 16.916 | −76.827 | 1.00 | 42.22 | D |
| ATOM | 14091 | CA | LYS | D | 407 | 49.746 | 17.349 | −77.696 | 1.00 | 37.82 | D |
| ATOM | 14092 | CB | LYS | D | 407 | 50.091 | 17.153 | −79.171 | 1.00 | 112.23 | D |
| ATOM | 14093 | CG | LYS | D | 407 | 51.258 | 17.984 | −79.666 | 1.00 | 98.33 | D |
| ATOM | 14094 | CD | LYS | D | 407 | 51.382 | 17.893 | −81.184 | 1.00 | 98.33 | D |
| ATOM | 14095 | CE | LYS | D | 407 | 52.653 | 18.566 | −81.689 | 1.00 | 98.33 | D |
| ATOM | 14096 | NZ | LYS | D | 407 | 53.891 | 17.870 | −81.221 | 1.00 | 98.33 | D |
| ATOM | 14097 | C | LYS | D | 407 | 48.579 | 16.463 | −77.345 | 1.00 | 47.13 | D |
| ATOM | 14098 | O | LYS | D | 407 | 48.772 | 15.276 | −77.083 | 1.00 | 46.07 | D |
| ATOM | 14099 | N | LYS | D | 408 | 47.376 | 17.030 | −77.301 | 1.00 | 51.43 | D |
| ATOM | 14100 | CA | LYS | D | 408 | 46.209 | 16.220 | −76.989 | 1.00 | 51.02 | D |
| ATOM | 14101 | CB | LYS | D | 408 | 45.010 | 17.096 | −76.658 | 1.00 | 55.14 | D |
| ATOM | 14102 | CG | LYS | D | 408 | 43.814 | 16.304 | −76.179 | 1.00 | 53.94 | D |
| ATOM | 14103 | CD | LYS | D | 408 | 42.768 | 17.213 | −75.592 | 1.00 | 53.94 | D |
| ATOM | 14104 | CE | LYS | D | 408 | 41.595 | 16.415 | −75.051 | 1.00 | 53.94 | D |
| ATOM | 14105 | NZ | LYS | D | 408 | 40.600 | 17.301 | −74.359 | 1.00 | 53.94 | D |
| ATOM | 14106 | C | LYS | D | 408 | 45.957 | 15.449 | −78.269 | 1.00 | 51.15 | D |
| ATOM | 14107 | O | LYS | D | 408 | 45.567 | 16.029 | −79.275 | 1.00 | 49.72 | D |
| ATOM | 14108 | N | ASP | D | 409 | 46.198 | 14.147 | −78.256 | 1.00 | 71.03 | D |
| ATOM | 14109 | CA | ASP | D | 409 | 46.005 | 13.398 | −79.484 | 1.00 | 71.21 | D |
| ATOM | 14110 | CB | ASP | D | 409 | 46.568 | 11.967 | −79.361 | 1.00 | 94.96 | D |
| ATOM | 14111 | CG | ASP | D | 409 | 46.180 | 11.285 | −78.074 | 1.00 | 87.78 | D |
| ATOM | 14112 | OD1 | ASP | D | 409 | 44.992 | 11.341 | −77.712 | 1.00 | 87.78 | D |
| ATOM | 14113 | OD2 | ASP | D | 409 | 47.062 | 10.678 | −77.433 | 1.00 | 87.78 | D |
| ATOM | 14114 | C | ASP | D | 409 | 44.564 | 13.380 | −79.977 | 1.00 | 71.12 | D |
| ATOM | 14115 | O | ASP | D | 409 | 43.639 | 13.705 | −79.234 | 1.00 | 74.85 | D |
| ATOM | 14116 | N | LYS | D | 410 | 44.415 | 13.028 | −81.257 | 1.00 | 85.72 | D |
| ATOM | 14117 | CA | LYS | D | 410 | 43.139 | 12.936 | −81.972 | 1.00 | 92.16 | D |
| ATOM | 14118 | CB | LYS | D | 410 | 43.187 | 11.782 | −82.980 | 1.00 | 160.70 | D |
| ATOM | 14119 | CG | LYS | D | 410 | 43.237 | 10.377 | −82.357 | 1.00 | 100.15 | D |
| ATOM | 14120 | CD | LYS | D | 410 | 44.595 | 10.059 | −81.721 | 1.00 | 100.15 | D |
| ATOM | 14121 | CE | LYS | D | 410 | 44.596 | 8.674 | −81.067 | 1.00 | 100.15 | D |
| ATOM | 14122 | NZ | LYS | D | 410 | 45.904 | 8.339 | −80.426 | 1.00 | 100.15 | D |
| ATOM | 14123 | C | LYS | D | 410 | 41.916 | 12.753 | −81.091 | 1.00 | 90.98 | D |
| ATOM | 14124 | O | LYS | D | 410 | 42.007 | 12.230 | −79.988 | 1.00 | 90.51 | D |
| ATOM | 14125 | N | ASP | D | 411 | 40.766 | 13.166 | −81.607 | 1.00 | 50.58 | D |
| ATOM | 14126 | CA | ASP | D | 411 | 39.495 | 13.076 | −80.888 | 1.00 | 48.09 | D |
| ATOM | 14127 | CB | ASP | D | 411 | 38.348 | 12.756 | −81.853 | 1.00 | 155.37 | D |
| ATOM | 14128 | CG | ASP | D | 411 | 37.736 | 14.002 | −82.464 | 1.00 | 155.37 | D |
| ATOM | 14129 | OD1 | ASP | D | 411 | 37.364 | 14.919 | −81.699 | 1.00 | 155.37 | D |
| ATOM | 14130 | OD2 | ASP | D | 411 | 37.618 | 14.061 | −83.706 | 1.00 | 155.37 | D |
| ATOM | 14131 | C | ASP | D | 411 | 39.467 | 12.084 | −79.736 | 1.00 | 49.70 | D |
| ATOM | 14132 | O | ASP | D | 411 | 39.023 | 10.947 | −79.896 | 1.00 | 53.88 | D |
| ATOM | 14133 | N | ALA | D | 412 | 39.930 | 12.548 | −78.572 | 1.00 | 74.19 | D |
| ATOM | 14134 | CA | ALA | D | 412 | 39.983 | 11.748 | −77.354 | 1.00 | 87.21 | D |
| ATOM | 14135 | CB | ALA | D | 412 | 40.977 | 10.627 | −77.520 | 1.00 | 40.47 | D |
| ATOM | 14136 | C | ALA | D | 412 | 40.368 | 12.615 | −76.164 | 1.00 | 81.91 | D |
| ATOM | 14137 | O | ALA | D | 412 | 40.738 | 13.779 | −76.320 | 1.00 | 76.96 | D |
| ATOM | 14138 | N | ASN | D | 413 | 40.284 | 12.036 | −74.975 | 1.00 | 66.46 | D |
| ATOM | 14139 | CA | ASN | D | 413 | 40.608 | 12.759 | −73.758 | 1.00 | 59.27 | D |
| ATOM | 14140 | CB | ASN | D | 413 | 39.763 | 12.248 | −72.596 | 1.00 | 60.51 | D |
| ATOM | 14141 | CG | ASN | D | 413 | 38.276 | 12.462 | −72.815 | 1.00 | 60.51 | D |
| ATOM | 14142 | OD1 | ASN | D | 413 | 37.866 | 13.511 | −73.326 | 1.00 | 60.51 | D |
| ATOM | 14143 | ND2 | ASN | D | 413 | 37.452 | 11.474 | −72.410 | 1.00 | 60.51 | D |
| ATOM | 14144 | C | ASN | D | 413 | 42.063 | 12.592 | −73.389 | 1.00 | 63.85 | D |
| ATOM | 14145 | O | ASN | D | 413 | 42.589 | 13.308 | −72.542 | 1.00 | 63.54 | D |
| ATOM | 14146 | N | THR | D | 414 | 42.709 | 11.639 | −74.039 | 1.00 | 40.63 | D |
| ATOM | 14147 | CA | THR | D | 414 | 44.097 | 11.315 | −73.776 | 1.00 | 43.67 | D |
| ATOM | 14148 | CB | THR | D | 414 | 44.337 | 9.857 | −74.169 | 1.00 | 97.88 | D |
| ATOM | 14149 | OG1 | THR | D | 414 | 45.693 | 9.523 | −73.907 | 1.00 | 68.37 | D |
| ATOM | 14150 | CG2 | THR | D | 414 | 44.031 | 9.630 | −75.633 | 1.00 | 68.37 | D |
| ATOM | 14151 | C | THR | D | 414 | 45.132 | 12.247 | −74.440 | 1.00 | 43.13 | D |
| ATOM | 14152 | O | THR | D | 414 | 44.795 | 13.019 | −75.341 | 1.00 | 44.20 | D |
| ATOM | 14153 | N | TYR | D | 415 | 46.381 | 12.192 | −73.961 | 1.00 | 45.13 | D |
| ATOM | 14154 | CA | TYR | D | 415 | 47.472 | 13.024 | −74.481 | 1.00 | 46.16 | D |
| ATOM | 14155 | CB | TYR | D | 415 | 47.927 | 14.040 | −73.434 | 1.00 | 73.80 | D |
| ATOM | 14156 | CG | TYR | D | 415 | 46.904 | 15.090 | −73.074 | 1.00 | 40.74 | D |
| ATOM | 14157 | CD1 | TYR | D | 415 | 45.749 | 14.752 | −72.373 | 1.00 | 40.74 | D |
| ATOM | 14158 | CE1 | TYR | D | 415 | 44.796 | 15.713 | −72.059 | 1.00 | 40.74 | D |
| ATOM | 14159 | CD2 | TYR | D | 415 | 47.083 | 16.427 | −73.451 | 1.00 | 40.74 | D |
| ATOM | 14160 | CE2 | TYR | D | 415 | 46.140 | 17.393 | −73.140 | 1.00 | 40.74 | D |
| ATOM | 14161 | CZ | TYR | D | 415 | 44.996 | 17.033 | −72.446 | 1.00 | 40.74 | D |
| ATOM | 14162 | OH | TYR | D | 415 | 44.046 | 17.984 | −72.138 | 1.00 | 40.74 | D |
| ATOM | 14163 | C | TYR | D | 415 | 48.686 | 12.184 | −74.871 | 1.00 | 53.72 | D |
| ATOM | 14164 | O | TYR | D | 415 | 48.816 | 11.030 | −74.450 | 1.00 | 48.37 | D |
| ATOM | 14165 | N | SER | D | 416 | 49.580 | 12.773 | −75.663 | 1.00 | 45.40 | D |
| ATOM | 14166 | CA | SER | D | 416 | 50.795 | 12.093 | −76.104 | 1.00 | 43.35 | D |
| ATOM | 14167 | CB | SER | D | 416 | 50.660 | 11.646 | −77.548 | 1.00 | 80.50 | D |
| ATOM | 14168 | OG | SER | D | 416 | 49.795 | 10.534 | −77.632 | 1.00 | 56.88 | D |
| ATOM | 14169 | C | SER | D | 416 | 52.004 | 12.998 | −75.957 | 1.00 | 49.54 | D |
| ATOM | 14170 | O | SER | D | 416 | 51.901 | 14.226 | −76.067 | 1.00 | 47.91 | D |
| ATOM | 14171 | N | LEU | D | 417 | 53.158 | 12.381 | −75.724 | 1.00 | 37.53 | D |
| ATOM | 14172 | CA | LEU | D | 417 | 54.396 | 13.119 | −75.508 | 1.00 | 36.43 | D |
| ATOM | 14173 | CB | LEU | D | 417 | 54.711 | 13.137 | −74.014 | 1.00 | 81.68 | D |
| ATOM | 14174 | CG | LEU | D | 417 | 56.192 | 13.282 | −73.664 | 1.00 | 39.79 | D |
| ATOM | 14175 | CD1 | LEU | D | 417 | 56.620 | 14.738 | −73.818 | 1.00 | 39.79 | D |
| ATOM | 14176 | CD2 | LEU | D | 417 | 56.431 | 12.810 | −72.261 | 1.00 | 39.79 | D |
| ATOM | 14177 | C | LEU | D | 417 | 55.594 | 12.546 | −76.252 | 1.00 | 47.94 | D |
| ATOM | 14178 | O | LEU | D | 417 | 55.675 | 11.351 | −76.484 | 1.00 | 43.04 | D |
| ATOM | 14179 | N | THR | D | 418 | 56.535 | 13.418 | −76.597 | 1.00 | 54.87 | D |
| ATOM | 14180 | CA | THR | D | 418 | 57.747 | 13.024 | −77.299 | 1.00 | 47.93 | D |
| ATOM | 14181 | CB | THR | D | 418 | 57.666 | 13.370 | −78.797 | 1.00 | 88.90 | D |
| ATOM | 14182 | OG1 | THR | D | 418 | 56.750 | 12.480 | −79.442 | 1.00 | 57.31 | D |
| ATOM | 14183 | CG2 | THR | D | 418 | 59.021 | 13.236 | −79.455 | 1.00 | 57.31 | D |
| ATOM | 14184 | C | THR | D | 418 | 58.915 | 13.778 | −76.703 | 1.00 | 63.12 | D |
| ATOM | 14185 | O | THR | D | 418 | 58.992 | 14.995 | −76.829 | 1.00 | 56.59 | D |
| ATOM | 14186 | N | ASP | D | 419 | 59.830 | 13.064 | −76.058 | 1.00 | 51.14 | D |
| ATOM | 14187 | CA | ASP | D | 419 | 60.982 | 13.727 | −75.463 | 1.00 | 56.25 | D |
| ATOM | 14188 | CB | ASP | D | 419 | 61.037 | 13.449 | −73.964 | 1.00 | 95.02 | D |
| ATOM | 14189 | CG | ASP | D | 419 | 61.981 | 14.382 | −73.243 | 1.00 | 72.61 | D |
| ATOM | 14190 | OD1 | ASP | D | 419 | 61.899 | 15.606 | −73.487 | 1.00 | 72.61 | D |
| ATOM | 14191 | OD2 | ASP | D | 419 | 62.799 | 13.902 | −72.433 | 1.00 | 72.61 | D |
| ATOM | 14192 | C | ASP | D | 419 | 62.269 | 13.272 | −76.135 | 1.00 | 60.34 | D |
| ATOM | 14193 | O | ASP | D | 419 | 62.552 | 12.080 | −76.194 | 1.00 | 55.23 | D |
| ATOM | 14194 | N | LEU | D | 420 | 63.046 | 14.229 | −76.641 | 1.00 | 74.08 | D |
| ATOM | 14195 | CA | LEU | D | 420 | 64.295 | 13.934 | −77.339 | 1.00 | 78.77 | D |
| ATOM | 14196 | CB | LEU | D | 420 | 64.343 | 14.745 | −78.637 | 1.00 | 127.61 | D |
| ATOM | 14197 | CG | LEU | D | 420 | 65.275 | 14.261 | −79.744 | 1.00 | 86.56 | D |
| ATOM | 14198 | CD1 | LEU | D | 420 | 64.789 | 12.916 | −80.252 | 1.00 | 86.56 | D |
| ATOM | 14199 | CD2 | LEU | D | 420 | 65.310 | 15.275 | −80.875 | 1.00 | 86.56 | D |
| ATOM | 14200 | C | LEU | D | 420 | 65.529 | 14.249 | −76.485 | 1.00 | 81.34 | D |
| ATOM | 14201 | O | LEU | D | 420 | 65.475 | 15.100 | −75.601 | 1.00 | 80.66 | D |
| ATOM | 14202 | N | VAL | D | 421 | 66.640 | 13.561 | −76.750 | 1.00 | 70.23 | D |
| ATOM | 14203 | CA | VAL | D | 421 | 67.883 | 13.779 | −76.004 | 1.00 | 68.74 | D |
| ATOM | 14204 | CB | VAL | D | 421 | 68.013 | 12.784 | −74.842 | 1.00 | 111.21 | D |
| ATOM | 14205 | CG1 | VAL | D | 421 | 69.357 | 12.933 | −74.165 | 1.00 | 66.64 | D |
| ATOM | 14206 | CG2 | VAL | D | 421 | 66.931 | 13.042 | −73.849 | 1.00 | 66.64 | D |
| ATOM | 14207 | C | VAL | D | 421 | 69.126 | 13.659 | −76.878 | 1.00 | 71.00 | D |
| ATOM | 14208 | O | VAL | D | 421 | 69.241 | 12.737 | −77.680 | 1.00 | 66.60 | D |
| ATOM | 14209 | N | ARG | D | 422 | 70.053 | 14.596 | −76.709 | 1.00 | 80.79 | D |
| ATOM | 14210 | CA | ARG | D | 422 | 71.301 | 14.623 | −77.468 | 1.00 | 82.92 | D |
| ATOM | 14211 | CB | ARG | D | 422 | 71.860 | 16.047 | −77.466 | 1.00 | 169.18 | D |
| ATOM | 14212 | CG | ARG | D | 422 | 72.128 | 16.635 | −78.835 | 1.00 | 129.77 | D |
| ATOM | 14213 | CD | ARG | D | 422 | 73.423 | 16.115 | −79.427 | 1.00 | 129.77 | D |
| ATOM | 14214 | NE | ARG | D | 422 | 73.748 | 16.803 | −80.675 | 1.00 | 129.77 | D |
| ATOM | 14215 | CZ | ARG | D | 422 | 74.886 | 16.648 | −81.346 | 1.00 | 129.77 | D |
| ATOM | 14216 | NH1 | ARG | D | 422 | 75.821 | 15.822 | −80.890 | 1.00 | 129.77 | D |
| ATOM | 14217 | NH2 | ARG | D | 422 | 75.092 | 17.324 | −82.472 | 1.00 | 129.77 | D |
| ATOM | 14218 | C | ARG | D | 422 | 72.308 | 13.673 | −76.819 | 1.00 | 80.15 | D |
| ATOM | 14219 | O | ARG | D | 422 | 72.926 | 14.014 | −75.812 | 1.00 | 70.82 | D |
| ATOM | 14220 | N | THR | D | 423 | 72.474 | 12.483 | −77.389 | 1.00 | 87.95 | D |
| ATOM | 14221 | CA | THR | D | 423 | 73.407 | 11.499 | −76.838 | 1.00 | 97.60 | D |
| ATOM | 14222 | CB | THR | D | 423 | 73.209 | 10.100 | −77.481 | 1.00 | 126.21 | D |
| ATOM | 14223 | OG1 | THR | D | 423 | 71.853 | 9.683 | −77.310 | 1.00 | 82.00 | D |
| ATOM | 14224 | CG2 | THR | D | 423 | 74.116 | 9.063 | −76.825 | 1.00 | 82.00 | D |
| ATOM | 14225 | C | THR | D | 423 | 74.861 | 11.901 | −77.041 | 1.00 | 95.82 | D |
| ATOM | 14226 | O | THR | D | 423 | 75.596 | 11.208 | −77.737 | 1.00 | 94.31 | D |
| ATOM | 14227 | N | THR | D | 424 | 75.281 | 13.012 | −76.442 | 1.00 | 164.73 | D |
| ATOM | 14228 | CA | THR | D | 424 | 76.664 | 13.458 | −76.581 | 1.00 | 164.73 | D |
| ATOM | 14229 | CB | THR | D | 424 | 76.896 | 14.811 | −75.878 | 1.00 | 200.84 | D |
| ATOM | 14230 | OG1 | THR | D | 424 | 75.994 | 15.786 | −76.416 | 1.00 | 148.06 | D |
| ATOM | 14231 | CG2 | THR | D | 424 | 78.329 | 15.291 | −76.095 | 1.00 | 148.06 | D |
| ATOM | 14232 | C | THR | D | 424 | 77.580 | 12.404 | −75.966 | 1.00 | 164.73 | D |
| ATOM | 14233 | O | THR | D | 424 | 77.159 | 11.637 | −75.100 | 1.00 | 164.73 | D |
| ATOM | 14234 | N | SER | D | 425 | 78.830 | 12.366 | −76.416 | 1.00 | 158.56 | D |
| ATOM | 14235 | CA | SER | D | 425 | 79.789 | 11.386 | −75.920 | 1.00 | 158.56 | D |
| ATOM | 14236 | CB | SER | D | 425 | 80.991 | 11.313 | −76.863 | 1.00 | 186.62 | D |
| ATOM | 14237 | OG | SER | D | 425 | 81.877 | 10.283 | −76.467 | 1.00 | 82.56 | D |
| ATOM | 14238 | C | SER | D | 425 | 80.275 | 11.654 | −74.496 | 1.00 | 158.56 | D |
| ATOM | 14239 | O | SER | D | 425 | 81.070 | 10.885 | −73.955 | 1.00 | 158.56 | D |
| ATOM | 14240 | N | LYS | D | 426 | 79.796 | 12.739 | −73.892 | 1.00 | 159.25 | D |
| ATOM | 14241 | CA | LYS | D | 426 | 80.190 | 13.097 | −72.531 | 1.00 | 159.25 | D |
| ATOM | 14242 | CB | LYS | D | 426 | 79.451 | 14.358 | −72.084 | 1.00 | 176.08 | D |
| ATOM | 14243 | CG | LYS | D | 426 | 79.563 | 15.520 | −73.044 | 1.00 | 120.38 | D |
| ATOM | 14244 | CD | LYS | D | 426 | 78.657 | 16.651 | −72.604 | 1.00 | 120.38 | D |
| ATOM | 14245 | CE | LYS | D | 426 | 78.629 | 17.766 | −73.630 | 1.00 | 120.38 | D |
| ATOM | 14246 | NZ | LYS | D | 426 | 77.700 | 18.852 | −73.215 | 1.00 | 120.38 | D |
| ATOM | 14247 | C | LYS | D | 426 | 79.877 | 11.968 | −71.551 | 1.00 | 159.25 | D |
| ATOM | 14248 | O | LYS | D | 426 | 80.655 | 11.685 | −70.639 | 1.00 | 159.25 | D |
| ATOM | 14249 | N | MET | D | 427 | 78.729 | 11.329 | −71.750 | 1.00 | 168.82 | D |
| ATOM | 14250 | CA | MET | D | 427 | 78.283 | 10.238 | −70.890 | 1.00 | 168.82 | D |
| ATOM | 14251 | CB | MET | D | 427 | 76.844 | 9.852 | −71.242 | 1.00 | 120.65 | D |
| ATOM | 14252 | CG | MET | D | 427 | 76.620 | 9.570 | −72.717 | 1.00 | 120.65 | D |
| ATOM | 14253 | SD | MET | D | 427 | 74.900 | 9.190 | −73.074 | 1.00 | 120.65 | D |
| ATOM | 14254 | CE | MET | D | 427 | 74.164 | 10.835 | −73.056 | 1.00 | 120.65 | D |
| ATOM | 14255 | C | MET | D | 427 | 79.169 | 9.004 | −70.968 | 1.00 | 168.82 | D |
| ATOM | 14256 | O | MET | D | 427 | 80.312 | 9.073 | −71.420 | 1.00 | 168.82 | D |
| ATOM | 14257 | N | SER | D | 428 | 78.630 | 7.874 | −70.521 | 1.00 | 100.78 | D |
| ATOM | 14258 | CA | SER | D | 428 | 79.374 | 6.622 | −70.534 | 1.00 | 102.43 | D |
| ATOM | 14259 | CB | SER | D | 428 | 80.523 | 6.690 | −69.527 | 1.00 | 115.17 | D |
| ATOM | 14260 | OG | SER | D | 428 | 80.028 | 6.847 | −68.209 | 1.00 | 111.88 | D |
| ATOM | 14261 | C | SER | D | 428 | 78.508 | 5.404 | −70.219 | 1.00 | 98.38 | D |
| ATOM | 14262 | O | SER | D | 428 | 79.005 | 4.427 | −69.661 | 1.00 | 98.26 | D |
| ATOM | 14263 | N | SER | D | 429 | 77.225 | 5.455 | −70.574 | 1.00 | 130.90 | D |
| ATOM | 14264 | CA | SER | D | 429 | 76.318 | 4.336 | −70.314 | 1.00 | 129.57 | D |
| ATOM | 14265 | CB | SER | D | 429 | 75.883 | 4.328 | −68.849 | 1.00 | 134.49 | D |
| ATOM | 14266 | OG | SER | D | 429 | 76.981 | 4.126 | −67.983 | 1.00 | 134.49 | D |
| ATOM | 14267 | C | SER | D | 429 | 75.068 | 4.360 | −71.180 | 1.00 | 127.95 | D |
| ATOM | 14268 | O | SER | D | 429 | 74.299 | 3.401 | −71.190 | 1.00 | 116.65 | D |
| ATOM | 14269 | N | GLN | D | 430 | 74.867 | 5.448 | −71.912 | 1.00 | 80.61 | D |
| ATOM | 14270 | CA | GLN | D | 430 | 73.681 | 5.594 | −72.750 | 1.00 | 80.61 | D |
| ATOM | 14271 | CB | GLN | D | 430 | 73.526 | 4.428 | −73.718 | 1.00 | 175.27 | D |
| ATOM | 14272 | CG | GLN | D | 430 | 72.335 | 4.613 | −74.629 | 1.00 | 104.26 | D |
| ATOM | 14273 | CD | GLN | D | 430 | 72.126 | 3.455 | −75.568 | 1.00 | 104.26 | D |
| ATOM | 14274 | OE1 | GLN | D | 430 | 71.765 | 2.355 | −75.147 | 1.00 | 104.26 | D |
| ATOM | 14275 | NE2 | GLN | D | 430 | 72.355 | 3.694 | −76.857 | 1.00 | 104.26 | D |
| ATOM | 14276 | C | GLN | D | 430 | 72.470 | 5.651 | −71.828 | 1.00 | 80.61 | D |
| ATOM | 14277 | O | GLN | D | 430 | 72.105 | 4.659 | −71.191 | 1.00 | 80.61 | D |
| ATOM | 14278 | N | PRO | D | 431 | 71.813 | 6.816 | −71.765 | 1.00 | 53.81 | D |
| ATOM | 14279 | CD | PRO | D | 431 | 71.902 | 7.959 | −72.686 | 1.00 | 40.84 | D |
| ATOM | 14280 | CA | PRO | D | 431 | 70.655 | 6.955 | −70.896 | 1.00 | 53.80 | D |
| ATOM | 14281 | CB | PRO | D | 431 | 70.258 | 8.410 | −71.096 | 1.00 | 139.80 | D |
| ATOM | 14282 | CG | PRO | D | 431 | 70.551 | 8.616 | −72.526 | 1.00 | 110.36 | D |
| ATOM | 14283 | C | PRO | D | 431 | 69.527 | 5.998 | −71.216 | 1.00 | 50.42 | D |
| ATOM | 14284 | O | PRO | D | 431 | 69.419 | 5.464 | −72.322 | 1.00 | 50.30 | D |
| ATOM | 14285 | N | VAL | D | 432 | 68.700 | 5.789 | −70.202 | 1.00 | 56.35 | D |
| ATOM | 14286 | CA | VAL | D | 432 | 67.521 | 4.956 | −70.288 | 1.00 | 58.73 | D |
| ATOM | 14287 | CB | VAL | D | 432 | 67.599 | 3.808 | −69.273 | 1.00 | 73.91 | D |
| ATOM | 14288 | CG1 | VAL | D | 432 | 67.664 | 4.351 | −67.874 | 1.00 | 45.99 | D |
| ATOM | 14289 | CG2 | VAL | D | 432 | 66.426 | 2.923 | −69.422 | 1.00 | 45.99 | D |
| ATOM | 14290 | C | VAL | D | 432 | 66.407 | 5.948 | −69.929 | 1.00 | 56.73 | D |
| ATOM | 14291 | O | VAL | D | 432 | 66.635 | 6.903 | −69.186 | 1.00 | 47.09 | D |
| ATOM | 14292 | N | CYS | D | 433 | 65.215 | 5.759 | −70.475 | 1.00 | 69.31 | D |
| ATOM | 14293 | CA | CYS | D | 433 | 64.130 | 6.690 | −70.190 | 1.00 | 65.19 | D |
| ATOM | 14294 | C | CYS | D | 433 | 63.256 | 6.251 | −69.019 | 1.00 | 62.83 | D |
| ATOM | 14295 | O | CYS | D | 433 | 62.707 | 5.147 | −69.030 | 1.00 | 63.57 | D |
| ATOM | 14296 | CB | CYS | D | 433 | 63.265 | 6.877 | −71.442 | 1.00 | 141.92 | D |
| ATOM | 14297 | SG | CYS | D | 433 | 61.958 | 8.137 | −71.276 | 1.00 | 89.03 | D |
| ATOM | 14298 | N | VAL | D | 434 | 63.150 | 7.105 | −67.998 | 1.00 | 48.00 | D |
| ATOM | 14299 | CA | VAL | D | 434 | 62.298 | 6.817 | −66.841 | 1.00 | 43.99 | D |
| ATOM | 14300 | CB | VAL | D | 434 | 62.996 | 7.103 | −65.522 | 1.00 | 55.64 | D |
| ATOM | 14301 | CG1 | VAL | D | 434 | 62.135 | 6.612 | −64.383 | 1.00 | 37.42 | D |
| ATOM | 14302 | CG2 | VAL | D | 434 | 64.332 | 6.422 | −65.492 | 1.00 | 37.42 | D |
| ATOM | 14303 | C | VAL | D | 434 | 61.062 | 7.713 | −66.929 | 1.00 | 54.39 | D |
| ATOM | 14304 | O | VAL | D | 434 | 61.164 | 8.937 | −66.888 | 1.00 | 45.45 | D |
| ATOM | 14305 | N | VAL | D | 435 | 59.894 | 7.093 | −67.040 | 1.00 | 41.24 | D |
| ATOM | 14306 | CA | VAL | D | 435 | 58.643 | 7.826 | −67.186 | 1.00 | 32.98 | D |
| ATOM | 14307 | CB | VAL | D | 435 | 57.780 | 7.188 | −68.309 | 1.00 | 53.68 | D |
| ATOM | 14308 | CG1 | VAL | D | 435 | 56.381 | 7.772 | −68.311 | 1.00 | 33.01 | D |
| ATOM | 14309 | CG2 | VAL | D | 435 | 58.447 | 7.409 | −69.654 | 1.00 | 33.01 | D |
| ATOM | 14310 | C | VAL | D | 435 | 57.813 | 7.884 | −65.913 | 1.00 | 40.90 | D |
| ATOM | 14311 | O | VAL | D | 435 | 57.443 | 6.848 | −65.367 | 1.00 | 41.49 | D |
| ATOM | 14312 | N | PHE | D | 436 | 57.519 | 9.102 | −65.453 | 1.00 | 40.14 | D |
| ATOM | 14313 | CA | PHE | D | 436 | 56.707 | 9.303 | −64.260 | 1.00 | 36.34 | D |
| ATOM | 14314 | CB | PHE | D | 436 | 57.328 | 10.357 | −63.359 | 1.00 | 71.75 | D |
| ATOM | 14315 | CG | PHE | D | 436 | 58.668 | 9.972 | −62.821 | 1.00 | 46.52 | D |
| ATOM | 14316 | CD1 | PHE | D | 436 | 59.830 | 10.231 | −63.551 | 1.00 | 46.52 | D |
| ATOM | 14317 | CD2 | PHE | D | 436 | 58.777 | 9.327 | −61.581 | 1.00 | 46.52 | D |
| ATOM | 14318 | CE1 | PHE | D | 436 | 61.085 | 9.856 | −63.055 | 1.00 | 46.52 | D |
| ATOM | 14319 | CE2 | PHE | D | 436 | 60.026 | 8.945 | −61.071 | 1.00 | 46.52 | D |
| ATOM | 14320 | CZ | PHE | D | 436 | 61.181 | 9.211 | −61.808 | 1.00 | 46.52 | D |
| ATOM | 14321 | C | PHE | D | 436 | 55.288 | 9.730 | −64.636 | 1.00 | 44.17 | D |
| ATOM | 14322 | O | PHE | D | 436 | 55.082 | 10.775 | −65.245 | 1.00 | 43.55 | D |
| ATOM | 14323 | N | HIS | D | 437 | 54.310 | 8.909 | −64.274 | 1.00 | 38.63 | D |
| ATOM | 14324 | CA | HIS | D | 437 | 52.914 | 9.191 | −64.574 | 1.00 | 39.70 | D |
| ATOM | 14325 | CB | HIS | D | 437 | 52.563 | 8.644 | −65.942 | 1.00 | 66.34 | D |
| ATOM | 14326 | CG | HIS | D | 437 | 51.154 | 8.912 | −66.339 | 1.00 | 42.71 | D |
| ATOM | 14327 | CD2 | HIS | D | 437 | 50.055 | 8.122 | −66.306 | 1.00 | 42.71 | D |
| ATOM | 14328 | ND1 | HIS | D | 437 | 50.735 | 10.139 | −66.810 | 1.00 | 42.71 | D |
| ATOM | 14329 | CE1 | HIS | D | 437 | 49.437 | 10.090 | −67.052 | 1.00 | 42.71 | D |
| ATOM | 14330 | NE2 | HIS | D | 437 | 49.000 | 8.878 | −66.753 | 1.00 | 42.71 | D |
| ATOM | 14331 | C | HIS | D | 437 | 52.027 | 8.536 | −63.521 | 1.00 | 44.01 | D |
| ATOM | 14332 | O | HIS | D | 437 | 52.464 | 7.624 | −62.833 | 1.00 | 46.23 | D |
| ATOM | 14333 | N | ASP | D | 438 | 50.788 | 8.995 | −63.384 | 1.00 | 46.45 | D |
| ATOM | 14334 | CA | ASP | D | 438 | 49.878 | 8.430 | −62.388 | 1.00 | 48.36 | D |
| ATOM | 14335 | CB | ASP | D | 438 | 48.522 | 9.121 | −62.462 | 1.00 | 99.14 | D |
| ATOM | 14336 | CG | ASP | D | 438 | 48.489 | 10.406 | −61.660 | 1.00 | 70.10 | D |
| ATOM | 14337 | OD1 | ASP | D | 438 | 49.576 | 10.968 | −61.398 | 1.00 | 70.10 | D |
| ATOM | 14338 | OD2 | ASP | D | 438 | 47.380 | 10.856 | −61.299 | 1.00 | 70.10 | D |
| ATOM | 14339 | C | ASP | D | 438 | 49.735 | 6.925 | −62.543 | 1.00 | 49.70 | D |
| ATOM | 14340 | O | ASP | D | 438 | 49.809 | 6.191 | −61.560 | 1.00 | 53.26 | D |
| ATOM | 14341 | N | THR | D | 439 | 49.515 | 6.461 | −63.767 | 1.00 | 75.03 | D |
| ATOM | 14342 | CA | THR | D | 439 | 49.440 | 5.029 | −64.021 | 1.00 | 72.38 | D |
| ATOM | 14343 | CB | THR | D | 439 | 48.773 | 4.746 | −65.376 | 1.00 | 61.32 | D |
| ATOM | 14344 | OG1 | THR | D | 439 | 49.564 | 5.299 | −66.433 | 1.00 | 47.13 | D |
| ATOM | 14345 | CG2 | THR | D | 439 | 47.401 | 5.392 | −65.423 | 1.00 | 47.13 | D |
| ATOM | 14346 | C | THR | D | 439 | 50.930 | 4.692 | −64.063 | 1.00 | 78.15 | D |
| ATOM | 14347 | O | THR | D | 439 | 51.720 | 5.516 | −64.512 | 1.00 | 78.49 | D |
| ATOM | 14348 | N | LEU | D | 440 | 51.332 | 3.515 | −63.599 | 1.00 | 56.19 | D |
| ATOM | 14349 | CA | LEU | D | 440 | 52.764 | 3.170 | −63.551 | 1.00 | 58.21 | D |
| ATOM | 14350 | CB | LEU | D | 440 | 53.357 | 2.926 | −64.968 | 1.00 | 72.12 | D |
| ATOM | 14351 | CG | LEU | D | 440 | 53.566 | 3.773 | −66.253 | 1.00 | 52.12 | D |
| ATOM | 14352 | CD1 | LEU | D | 440 | 52.238 | 4.047 | −66.886 | 1.00 | 52.12 | D |
| ATOM | 14353 | CD2 | LEU | D | 440 | 54.341 | 5.052 | −66.004 | 1.00 | 52.12 | D |
| ATOM | 14354 | C | LEU | D | 440 | 53.605 | 4.223 | −62.815 | 1.00 | 68.16 | D |
| ATOM | 14355 | O | LEU | D | 440 | 54.312 | 4.993 | −63.449 | 1.00 | 66.38 | D |
| ATOM | 14356 | N | GLU | D | 441 | 53.532 | 4.244 | −61.481 | 1.00 | 56.45 | D |
| ATOM | 14357 | CA | GLU | D | 441 | 54.265 | 5.215 | −60.652 | 1.00 | 56.45 | D |
| ATOM | 14358 | CB | GLU | D | 441 | 54.520 | 4.637 | −59.259 | 1.00 | 200.28 | D |
| ATOM | 14359 | CG | GLU | D | 441 | 53.254 | 4.401 | −58.456 | 1.00 | 123.64 | D |
| ATOM | 14360 | CD | GLU | D | 441 | 53.541 | 3.977 | −57.026 | 1.00 | 123.64 | D |
| ATOM | 14361 | OE1 | GLU | D | 441 | 54.236 | 2.953 | −56.835 | 1.00 | 123.64 | D |
| ATOM | 14362 | OE2 | GLU | D | 441 | 53.071 | 4.669 | −56.092 | 1.00 | 123.64 | D |
| ATOM | 14363 | C | GLU | D | 441 | 55.584 | 5.707 | −61.252 | 1.00 | 56.45 | D |
| ATOM | 14364 | O | GLU | D | 441 | 55.870 | 6.904 | −61.234 | 1.00 | 56.45 | D |
| ATOM | 14365 | N | ALA | D | 442 | 56.383 | 4.789 | −61.784 | 1.00 | 52.36 | D |
| ATOM | 14366 | CA | ALA | D | 442 | 57.655 | 5.145 | −62.408 | 1.00 | 50.58 | D |
| ATOM | 14367 | CB | ALA | D | 442 | 58.621 | 5.674 | −61.362 | 1.00 | 31.97 | D |
| ATOM | 14368 | C | ALA | D | 442 | 58.250 | 3.922 | −63.090 | 1.00 | 46.57 | D |
| ATOM | 14369 | O | ALA | D | 442 | 58.717 | 3.007 | −62.418 | 1.00 | 43.98 | D |
| ATOM | 14370 | N | GLN | D | 443 | 58.228 | 3.899 | −64.420 | 1.00 | 41.50 | D |
| ATOM | 14371 | CA | GLN | D | 443 | 58.775 | 2.777 | −65.180 | 1.00 | 44.78 | D |
| ATOM | 14372 | CB | GLN | D | 443 | 57.723 | 2.195 | −66.120 | 1.00 | 82.54 | D |
| ATOM | 14373 | CG | GLN | D | 443 | 56.919 | 1.082 | −65.504 | 1.00 | 65.77 | D |
| ATOM | 14374 | CD | GLN | D | 443 | 56.245 | 0.224 | −66.556 | 1.00 | 65.77 | D |
| ATOM | 14375 | OE1 | GLN | D | 443 | 56.890 | −0.231 | −67.501 | 1.00 | 65.77 | D |
| ATOM | 14376 | NE2 | GLN | D | 443 | 54.947 | −0.009 | −66.400 | 1.00 | 65.77 | D |
| ATOM | 14377 | C | GLN | D | 443 | 60.004 | 3.165 | −65.986 | 1.00 | 46.95 | D |
| ATOM | 14378 | O | GLN | D | 443 | 60.148 | 4.321 | −66.419 | 1.00 | 48.01 | D |
| ATOM | 14379 | N | VAL | D | 444 | 60.892 | 2.191 | −66.183 | 1.00 | 45.87 | D |
| ATOM | 14380 | CA | VAL | D | 444 | 62.111 | 2.416 | −66.952 | 1.00 | 43.02 | D |
| ATOM | 14381 | CB | VAL | D | 444 | 63.309 | 1.811 | −66.237 | 1.00 | 52.91 | D |
| ATOM | 14382 | CG1 | VAL | D | 444 | 64.597 | 2.382 | −66.806 | 1.00 | 45.21 | D |
| ATOM | 14383 | CG2 | VAL | D | 444 | 63.210 | 2.104 | −64.754 | 1.00 | 45.21 | D |
| ATOM | 14384 | C | VAL | D | 444 | 61.981 | 1.800 | −68.344 | 1.00 | 46.84 | D |
| ATOM | 14385 | O | VAL | D | 444 | 61.411 | 0.722 | −68.490 | 1.00 | 49.10 | D |
| ATOM | 14386 | N | TYR | D | 445 | 62.481 | 2.498 | −69.364 | 1.00 | 41.70 | D |
| ATOM | 14387 | CA | TYR | D | 445 | 62.412 | 2.021 | −70.749 | 1.00 | 40.77 | D |
| ATOM | 14388 | CB | TYR | D | 445 | 61.324 | 2.774 | −71.511 | 1.00 | 72.46 | D |
| ATOM | 14389 | CG | TYR | D | 445 | 59.959 | 2.616 | −70.889 | 1.00 | 44.88 | D |
| ATOM | 14390 | CD1 | TYR | D | 445 | 59.500 | 3.501 | −69.905 | 1.00 | 44.88 | D |
| ATOM | 14391 | CE1 | TYR | D | 445 | 58.242 | 3.349 | −69.320 | 1.00 | 44.88 | D |
| ATOM | 14392 | CD2 | TYR | D | 445 | 59.128 | 1.577 | −71.267 | 1.00 | 44.88 | D |
| ATOM | 14393 | CE2 | TYR | D | 445 | 57.869 | 1.408 | −70.690 | 1.00 | 44.88 | D |
| ATOM | 14394 | CZ | TYR | D | 445 | 57.428 | 2.294 | −69.722 | 1.00 | 44.88 | D |
| ATOM | 14395 | OH | TYR | D | 445 | 56.166 | 2.116 | −69.189 | 1.00 | 44.88 | D |
| ATOM | 14396 | C | TYR | D | 445 | 63.762 | 2.204 | −71.436 | 1.00 | 42.23 | D |
| ATOM | 14397 | O | TYR | D | 445 | 64.407 | 3.244 | −71.293 | 1.00 | 45.09 | D |
| ATOM | 14398 | N | THR | D | 446 | 64.175 | 1.204 | −72.207 | 1.00 | 40.58 | D |
| ATOM | 14399 | CA | THR | D | 446 | 65.478 | 1.243 | −72.857 | 1.00 | 47.56 | D |
| ATOM | 14400 | CB | THR | D | 446 | 66.325 | 0.075 | −72.354 | 1.00 | 54.92 | D |
| ATOM | 14401 | OG1 | THR | D | 446 | 65.635 | −1.141 | −72.632 | 1.00 | 43.07 | D |
| ATOM | 14402 | CG2 | THR | D | 446 | 66.529 | 0.157 | −70.864 | 1.00 | 43.07 | D |
| ATOM | 14403 | C | THR | D | 446 | 65.539 | 1.220 | −74.383 | 1.00 | 57.36 | D |
| ATOM | 14404 | O | THR | D | 446 | 64.604 | 1.606 | −75.072 | 1.00 | 61.03 | D |
| ATOM | 14405 | N | CYS | D | 447 | 66.701 | 0.763 | −74.856 | 1.00 | 83.41 | D |
| ATOM | 14406 | CA | CYS | D | 447 | 67.121 | 0.584 | −76.255 | 1.00 | 84.39 | D |
| ATOM | 14407 | C | CYS | D | 447 | 67.270 | 1.775 | −77.196 | 1.00 | 84.42 | D |
| ATOM | 14408 | O | CYS | D | 447 | 66.488 | 2.714 | −77.163 | 1.00 | 81.28 | D |
| ATOM | 14409 | CB | CYS | D | 447 | 66.289 | −0.509 | −76.946 | 1.00 | 100.43 | D |
| ATOM | 14410 | SG | CYS | D | 447 | 64.503 | −0.237 | −77.065 | 1.00 | 85.96 | D |
| ATOM | 14411 | N | SER | D | 448 | 68.320 | 1.704 | −78.011 | 1.00 | 183.09 | D |
| ATOM | 14412 | CA | SER | D | 448 | 68.647 | 2.729 | −78.992 | 1.00 | 183.09 | D |
| ATOM | 14413 | CB | SER | D | 448 | 69.947 | 3.436 | −78.610 | 1.00 | 200.84 | D |
| ATOM | 14414 | OG | SER | D | 448 | 69.834 | 4.048 | −77.338 | 1.00 | 200.84 | D |
| ATOM | 14415 | C | SER | D | 448 | 68.800 | 2.076 | −80.359 | 1.00 | 183.09 | D |
| ATOM | 14416 | O | SER | D | 448 | 68.227 | 1.015 | −80.603 | 1.00 | 183.09 | D |
| ATOM | 14417 | N | GLU | D | 449 | 69.592 | 2.692 | −81.235 | 1.00 | 151.34 | D |
| ATOM | 14418 | CA | GLU | D | 449 | 69.779 | 2.180 | −82.590 | 1.00 | 151.34 | D |
| ATOM | 14419 | CB | GLU | D | 449 | 70.153 | 0.691 | −82.575 | 1.00 | 113.21 | D |
| ATOM | 14420 | CG | GLU | D | 449 | 71.613 | 0.412 | −82.296 | 1.00 | 113.21 | D |
| ATOM | 14421 | CD | GLU | D | 449 | 72.129 | 1.216 | −81.127 | 1.00 | 113.21 | D |
| ATOM | 14422 | OE1 | GLU | D | 449 | 71.519 | 1.140 | −80.039 | 1.00 | 113.21 | D |
| ATOM | 14423 | OE2 | GLU | D | 449 | 73.143 | 1.925 | −81.300 | 1.00 | 113.21 | D |
| ATOM | 14424 | C | GLU | D | 449 | 68.442 | 2.361 | −83.289 | 1.00 | 151.34 | D |
| ATOM | 14425 | O | GLU | D | 449 | 68.371 | 2.791 | −84.440 | 1.00 | 151.34 | D |
| ATOM | 14426 | N | GLY | D | 450 | 67.384 | 2.038 | −82.557 | 1.00 | 68.88 | D |
| ATOM | 14427 | CA | GLY | D | 450 | 66.031 | 2.142 | −83.057 | 1.00 | 68.88 | D |
| ATOM | 14428 | C | GLY | D | 450 | 65.268 | 1.021 | −82.396 | 1.00 | 68.88 | D |
| ATOM | 14429 | O | GLY | D | 450 | 64.141 | 0.705 | −82.772 | 1.00 | 68.88 | D |
| ATOM | 14430 | N | CYS | D | 451 | 65.903 | 0.421 | −81.395 | 1.00 | 172.93 | D |
| ATOM | 14431 | CA | CYS | D | 451 | 65.322 | −0.694 | −80.666 | 1.00 | 172.93 | D |
| ATOM | 14432 | CB | CYS | D | 451 | 64.024 | −0.284 | −80.005 | 1.00 | 143.59 | D |
| ATOM | 14433 | SG | CYS | D | 451 | 64.291 | 0.924 | −78.697 | 1.00 | 143.59 | D |
| ATOM | 14434 | C | CYS | D | 451 | 65.045 | −1.825 | −81.621 | 1.00 | 172.93 | D |
| ATOM | 14435 | O | CYS | D | 451 | 63.875 | −2.257 | −81.679 | 1.00 | 172.93 | D |
| ATOM | 14436 | OXT | CYS | D | 451 | 66.002 | −2.254 | −82.297 | 1.00 | 115.96 | D |
| ATOM | 14437 | CB | ARG | E | 1 | 78.700 | −3.181 | −35.806 | 1.00 | 124.79 | E |
| ATOM | 14438 | CG | ARG | E | 1 | 77.750 | −4.084 | −35.003 | 1.00 | 124.79 | E |
| ATOM | 14439 | CD | ARG | E | 1 | 78.411 | −5.350 | −34.451 | 1.00 | 124.79 | E |
| ATOM | 14440 | NE | ARG | E | 1 | 78.937 | −5.185 | −33.092 | 1.00 | 124.79 | E |
| ATOM | 14441 | CZ | ARG | E | 1 | 78.190 | −4.951 | −32.012 | 1.00 | 124.79 | E |
| ATOM | 14442 | NH1 | ARG | E | 1 | 76.871 | −4.851 | −32.120 | 1.00 | 124.79 | E |
| ATOM | 14443 | NH2 | ARG | E | 1 | 78.762 | −4.817 | −30.820 | 1.00 | 124.79 | E |
| ATOM | 14444 | C | ARG | E | 1 | 79.441 | −1.892 | −33.768 | 1.00 | 200.84 | E |
| ATOM | 14445 | O | ARG | E | 1 | 79.032 | −2.526 | −32.797 | 1.00 | 200.84 | E |
| ATOM | 14446 | N | ARG | E | 1 | 80.861 | −3.712 | −34.714 | 1.00 | 200.84 | E |
| ATOM | 14447 | CA | ARG | E | 1 | 79.905 | −2.615 | −35.038 | 1.00 | 200.84 | E |
| ATOM | 14448 | N | THR | E | 2 | 79.513 | −0.564 | −33.778 | 1.00 | 192.58 | E |
| ATOM | 14449 | CA | THR | E | 2 | 79.109 | 0.242 | −32.625 | 1.00 | 192.58 | E |
| ATOM | 14450 | CB | THR | E | 2 | 79.678 | 1.687 | −32.713 | 1.00 | 143.02 | E |
| ATOM | 14451 | OG1 | THR | E | 2 | 81.105 | 1.642 | −32.839 | 1.00 | 141.76 | E |
| ATOM | 14452 | CG2 | THR | E | 2 | 79.308 | 2.489 | −31.467 | 1.00 | 144.91 | E |
| ATOM | 14453 | C | THR | E | 2 | 77.591 | 0.363 | −32.533 | 1.00 | 192.58 | E |
| ATOM | 14454 | O | THR | E | 2 | 76.976 | 1.020 | −33.374 | 1.00 | 192.58 | E |
| ATOM | 14455 | N | PRO | E | 3 | 76.961 | −0.276 | −31.526 | 1.00 | 200.84 | E |
| ATOM | 14456 | CD | PRO | E | 3 | 77.422 | −1.227 | −30.496 | 1.00 | 80.28 | E |
| ATOM | 14457 | CA | PRO | E | 3 | 75.506 | −0.117 | −31.478 | 1.00 | 200.84 | E |
| ATOM | 14458 | CB | PRO | E | 3 | 75.146 | −0.671 | −30.099 | 1.00 | 78.52 | E |
| ATOM | 14459 | CG | PRO | E | 3 | 76.103 | −1.824 | −29.976 | 1.00 | 78.39 | E |
| ATOM | 14460 | C | PRO | E | 3 | 75.184 | 1.368 | −31.646 | 1.00 | 200.84 | E |
| ATOM | 14461 | O | PRO | E | 3 | 74.183 | 1.723 | −32.270 | 1.00 | 200.84 | E |
| ATOM | 14462 | N | SER | E | 4 | 76.062 | 2.220 | −31.105 | 1.00 | 142.45 | E |
| ATOM | 14463 | CA | SER | E | 4 | 75.935 | 3.677 | −31.209 | 1.00 | 139.02 | E |
| ATOM | 14464 | CB | SER | E | 4 | 74.466 | 4.122 | −31.144 | 1.00 | 145.69 | E |
| ATOM | 14465 | OG | SER | E | 4 | 74.011 | 4.216 | −29.806 | 1.00 | 147.64 | E |
| ATOM | 14466 | C | SER | E | 4 | 76.709 | 4.453 | −30.150 | 1.00 | 139.36 | E |
| ATOM | 14467 | O | SER | E | 4 | 77.170 | 5.560 | −30.428 | 1.00 | 139.51 | E |
| ATOM | 14468 | N | ASP | E | 5 | 76.846 | 3.873 | −28.954 | 1.00 | 61.79 | E |
| ATOM | 14469 | CA | ASP | E | 5 | 77.528 | 4.514 | −27.822 | 1.00 | 61.90 | E |
| ATOM | 14470 | CB | ASP | E | 5 | 78.351 | 5.718 | −28.305 | 1.00 | 159.43 | E |
| ATOM | 14471 | CG | ASP | E | 5 | 79.030 | 6.463 | −27.180 | 1.00 | 159.43 | E |
| ATOM | 14472 | OD1 | ASP | E | 5 | 79.576 | 7.555 | −27.444 | 1.00 | 159.43 | E |
| ATOM | 14473 | OD2 | ASP | E | 5 | 79.027 | 5.963 | −26.038 | 1.00 | 157.99 | E |
| ATOM | 14474 | C | ASP | E | 5 | 76.473 | 4.972 | −26.792 | 1.00 | 59.40 | E |
| ATOM | 14475 | O | ASP | E | 5 | 76.771 | 5.137 | −25.607 | 1.00 | 62.91 | E |
| ATOM | 14476 | N | LYS | E | 6 | 75.238 | 5.169 | −27.254 | 1.00 | 55.01 | E |
| ATOM | 14477 | CA | LYS | E | 6 | 74.120 | 5.599 | −26.403 | 1.00 | 51.39 | E |
| ATOM | 14478 | CB | LYS | E | 6 | 72.931 | 6.057 | −27.253 | 1.00 | 60.14 | E |
| ATOM | 14479 | CG | LYS | E | 6 | 73.052 | 7.338 | −28.028 | 1.00 | 66.55 | E |
| ATOM | 14480 | CD | LYS | E | 6 | 71.809 | 7.472 | −28.905 | 1.00 | 70.16 | E |
| ATOM | 14481 | CE | LYS | E | 6 | 71.729 | 8.815 | −29.582 | 1.00 | 73.43 | E |
| ATOM | 14482 | NZ | LYS | E | 6 | 71.583 | 9.888 | −28.566 | 1.00 | 74.62 | E |
| ATOM | 14483 | C | LYS | E | 6 | 73.571 | 4.482 | −25.518 | 1.00 | 46.72 | E |
| ATOM | 14484 | O | LYS | E | 6 | 73.328 | 3.373 | −25.995 | 1.00 | 50.60 | E |
| ATOM | 14485 | N | PRO | E | 7 | 73.362 | 4.752 | −24.221 | 1.00 | 43.54 | E |
| ATOM | 14486 | CD | PRO | E | 7 | 73.772 | 5.875 | −23.369 | 1.00 | 44.85 | E |
| ATOM | 14487 | CA | PRO | E | 7 | 72.812 | 3.658 | −23.413 | 1.00 | 41.58 | E |
| ATOM | 14488 | CB | PRO | E | 7 | 72.731 | 4.251 | −21.997 | 1.00 | 46.85 | E |
| ATOM | 14489 | CG | PRO | E | 7 | 72.816 | 5.725 | −22.205 | 1.00 | 45.72 | E |
| ATOM | 14490 | C | PRO | E | 7 | 71.450 | 3.337 | −23.999 | 1.00 | 39.64 | E |
| ATOM | 14491 | O | PRO | E | 7 | 70.674 | 4.239 | −24.311 | 1.00 | 40.65 | E |
| ATOM | 14492 | N | VAL | E | 8 | 71.171 | 2.052 | −24.173 | 1.00 | 37.95 | E |
| ATOM | 14493 | CA | VAL | E | 8 | 69.911 | 1.629 | −24.759 | 1.00 | 39.11 | E |
| ATOM | 14494 | CB | VAL | E | 8 | 70.019 | 1.619 | −26.301 | 1.00 | 42.88 | E |
| ATOM | 14495 | CG1 | VAL | E | 8 | 71.154 | 0.696 | −26.728 | 1.00 | 43.46 | E |
| ATOM | 14496 | CG2 | VAL | E | 8 | 68.702 | 1.171 | −26.930 | 1.00 | 45.58 | E |
| ATOM | 14497 | C | VAL | E | 8 | 69.539 | 0.235 | −24.295 | 1.00 | 41.61 | E |
| ATOM | 14498 | O | VAL | E | 8 | 70.397 | −0.530 | −23.839 | 1.00 | 40.85 | E |
| ATOM | 14499 | N | ALA | E | 9 | 68.261 | −0.097 | −24.419 | 1.00 | 36.99 | E |
| ATOM | 14500 | CA | ALA | E | 9 | 67.807 | −1.413 | −24.029 | 1.00 | 43.23 | E |
| ATOM | 14501 | CB | ALA | E | 9 | 67.830 | −1.550 | −22.518 | 1.00 | 37.94 | E |
| ATOM | 14502 | C | ALA | E | 9 | 66.414 | −1.687 | −24.535 | 1.00 | 44.21 | E |
| ATOM | 14503 | O | ALA | E | 9 | 65.530 | −0.834 | −24.458 | 1.00 | 42.19 | E |
| ATOM | 14504 | N | HIS | E | 10 | 66.229 | −2.887 | −25.061 | 1.00 | 39.55 | E |
| ATOM | 14505 | CA | HIS | E | 10 | 64.939 | −3.322 | −25.542 | 1.00 | 40.42 | E |
| ATOM | 14506 | CB | HIS | E | 10 | 64.850 | −3.254 | −27.065 | 1.00 | 35.06 | E |
| ATOM | 14507 | CG | HIS | E | 10 | 63.511 | −3.654 | −27.597 | 1.00 | 36.55 | E |
| ATOM | 14508 | CD2 | HIS | E | 10 | 63.067 | −4.829 | −28.104 | 1.00 | 34.28 | E |
| ATOM | 14509 | ND1 | HIS | E | 10 | 62.424 | −2.809 | −27.586 | 1.00 | 36.69 | E |
| ATOM | 14510 | CE1 | HIS | E | 10 | 61.367 | −3.444 | −28.066 | 1.00 | 37.04 | E |
| ATOM | 14511 | NE2 | HIS | E | 10 | 61.730 | −4.671 | −28.387 | 1.00 | 34.52 | E |
| ATOM | 14512 | C | HIS | E | 10 | 64.856 | −4.770 | −25.079 | 1.00 | 38.63 | E |
| ATOM | 14513 | O | HIS | E | 10 | 65.418 | −5.671 | −25.704 | 1.00 | 36.79 | E |
| ATOM | 14514 | N | VAL | E | 11 | 64.188 | −4.985 | −23.958 | 1.00 | 43.05 | E |
| ATOM | 14515 | CA | VAL | E | 11 | 64.050 | −6.324 | −23.425 | 1.00 | 45.63 | E |
| ATOM | 14516 | CB | VAL | E | 11 | 64.295 | −6.353 | −21.914 | 1.00 | 42.38 | E |
| ATOM | 14517 | CG1 | VAL | E | 11 | 65.718 | −5.888 | −21.640 | 1.00 | 38.44 | E |
| ATOM | 14518 | CG2 | VAL | E | 11 | 63.285 | −5.465 | −21.187 | 1.00 | 44.36 | E |
| ATOM | 14519 | C | VAL | E | 11 | 62.669 | −6.853 | −23.744 | 1.00 | 44.24 | E |
| ATOM | 14520 | O | VAL | E | 11 | 61.702 | −6.088 | −23.921 | 1.00 | 41.25 | E |
| ATOM | 14521 | N | VAL | E | 12 | 62.579 | −8.171 | −23.820 | 1.00 | 57.28 | E |
| ATOM | 14522 | CA | VAL | E | 12 | 61.330 | −8.801 | −24.182 | 1.00 | 55.45 | E |
| ATOM | 14523 | CB | VAL | E | 12 | 61.497 | −9.454 | −25.558 | 1.00 | 62.15 | E |
| ATOM | 14524 | CG1 | VAL | E | 12 | 62.353 | −10.695 | −25.440 | 1.00 | 55.95 | E |
| ATOM | 14525 | CG2 | VAL | E | 12 | 60.155 | −9.747 | −26.158 | 1.00 | 65.62 | E |
| ATOM | 14526 | C | VAL | E | 12 | 60.938 | −9.823 | −23.120 | 1.00 | 53.41 | E |
| ATOM | 14527 | O | VAL | E | 12 | 61.784 | −10.263 | −22.329 | 1.00 | 53.25 | E |
| ATOM | 14528 | N | ALA | E | 13 | 59.658 | −10.187 | −23.095 | 1.00 | 53.98 | E |
| ATOM | 14529 | CA | ALA | E | 13 | 59.139 | −11.132 | −22.105 | 1.00 | 53.65 | E |
| ATOM | 14530 | CB | ALA | E | 13 | 57.618 | −11.056 | −22.052 | 1.00 | 27.81 | E |
| ATOM | 14531 | C | ALA | E | 13 | 59.562 | −12.564 | −22.344 | 1.00 | 57.63 | E |
| ATOM | 14532 | O | ALA | E | 13 | 59.575 | −13.025 | −23.476 | 1.00 | 55.14 | E |
| ATOM | 14533 | N | ASN | E | 14 | 59.897 | −13.258 | −21.260 | 1.00 | 71.12 | E |
| ATOM | 14534 | CA | ASN | E | 14 | 60.317 | −14.653 | −21.307 | 1.00 | 72.24 | E |
| ATOM | 14535 | CB | ASN | E | 14 | 61.129 | −14.979 | −20.060 | 1.00 | 90.93 | E |
| ATOM | 14536 | CG | ASN | E | 14 | 61.601 | −16.413 | −20.033 | 1.00 | 92.92 | E |
| ATOM | 14537 | OD1 | ASN | E | 14 | 62.207 | −16.855 | −19.059 | 1.00 | 92.50 | E |
| ATOM | 14538 | ND2 | ASN | E | 14 | 61.330 | −17.149 | −21.106 | 1.00 | 95.04 | E |
| ATOM | 14539 | C | ASN | E | 14 | 59.083 | −15.553 | −21.370 | 1.00 | 75.12 | E |
| ATOM | 14540 | O | ASN | E | 14 | 58.290 | −15.592 | −20.428 | 1.00 | 75.64 | E |
| ATOM | 14541 | N | PRO | E | 15 | 58.912 | −16.301 | −22.474 | 1.00 | 74.24 | E |
| ATOM | 14542 | CD | PRO | E | 15 | 59.751 | −16.379 | −23.682 | 1.00 | 48.18 | E |
| ATOM | 14543 | CA | PRO | E | 15 | 57.742 | −17.180 | −22.599 | 1.00 | 77.84 | E |
| ATOM | 14544 | CB | PRO | E | 15 | 57.704 | −17.515 | −24.100 | 1.00 | 52.52 | E |
| ATOM | 14545 | CG | PRO | E | 15 | 58.717 | −16.560 | −24.751 | 1.00 | 50.79 | E |
| ATOM | 14546 | C | PRO | E | 15 | 57.861 | −18.438 | −21.740 | 1.00 | 79.05 | E |
| ATOM | 14547 | O | PRO | E | 15 | 56.865 | −19.084 | −21.422 | 1.00 | 81.08 | E |
| ATOM | 14548 | N | GLN | E | 16 | 59.089 | −18.773 | −21.367 | 1.00 | 95.58 | E |
| ATOM | 14549 | CA | GLN | E | 16 | 59.351 | −19.958 | −20.563 | 1.00 | 94.53 | E |
| ATOM | 14550 | CB | GLN | E | 16 | 60.817 | −20.375 | −20.740 | 1.00 | 142.00 | E |
| ATOM | 14551 | CG | GLN | E | 16 | 61.133 | −21.821 | −20.377 | 1.00 | 144.93 | E |
| ATOM | 14552 | CD | GLN | E | 16 | 60.344 | −22.821 | −21.208 | 1.00 | 146.72 | E |
| ATOM | 14553 | OE1 | GLN | E | 16 | 60.202 | −22.665 | −22.423 | 1.00 | 148.84 | E |
| ATOM | 14554 | NE2 | GLN | E | 16 | 59.837 | −23.863 | −20.556 | 1.00 | 145.37 | E |
| ATOM | 14555 | C | GLN | E | 16 | 59.040 | −19.743 | −19.075 | 1.00 | 91.20 | E |
| ATOM | 14556 | O | GLN | E | 16 | 59.255 | −20.639 | −18.255 | 1.00 | 91.77 | E |
| ATOM | 14557 | N | ALA | E | 17 | 58.534 | −18.560 | −18.728 | 1.00 | 97.79 | E |
| ATOM | 14558 | CA | ALA | E | 17 | 58.203 | −18.246 | −17.334 | 1.00 | 101.55 | E |
| ATOM | 14559 | CB | ALA | E | 17 | 59.013 | −17.049 | −16.850 | 1.00 | 51.08 | E |
| ATOM | 14560 | C | ALA | E | 17 | 56.718 | −17.961 | −17.179 | 1.00 | 106.05 | E |
| ATOM | 14561 | O | ALA | E | 17 | 56.318 | −16.855 | −16.818 | 1.00 | 107.69 | E |
| ATOM | 14562 | N | GLU | E | 18 | 55.909 | −18.975 | −17.451 | 1.00 | 84.99 | E |
| ATOM | 14563 | CA | GLU | E | 18 | 54.463 | −18.860 | −17.357 | 1.00 | 86.99 | E |
| ATOM | 14564 | CB | GLU | E | 18 | 53.818 | −20.239 | −17.506 | 1.00 | 129.07 | E |
| ATOM | 14565 | CG | GLU | E | 18 | 54.233 | −21.233 | −16.432 | 1.00 | 129.07 | E |
| ATOM | 14566 | CD | GLU | E | 18 | 53.469 | −22.542 | −16.520 | 1.00 | 129.07 | E |
| ATOM | 14567 | OE1 | GLU | E | 18 | 52.226 | −22.514 | −16.387 | 1.00 | 129.07 | E |
| ATOM | 14568 | OE2 | GLU | E | 18 | 54.110 | −23.598 | −16.721 | 1.00 | 129.07 | E |
| ATOM | 14569 | C | GLU | E | 18 | 54.009 | −18.235 | −16.047 | 1.00 | 86.65 | E |
| ATOM | 14570 | O | GLU | E | 18 | 54.661 | −18.386 | −15.014 | 1.00 | 86.26 | E |
| ATOM | 14571 | N | GLY | E | 19 | 52.885 | −17.525 | −16.114 | 1.00 | 94.26 | E |
| ATOM | 14572 | CA | GLY | E | 19 | 52.319 | −16.893 | −14.940 | 1.00 | 91.75 | E |
| ATOM | 14573 | C | GLY | E | 19 | 52.970 | −15.606 | −14.485 | 1.00 | 90.25 | E |
| ATOM | 14574 | O | GLY | E | 19 | 52.433 | −14.933 | −13.612 | 1.00 | 90.26 | E |
| ATOM | 14575 | N | GLN | E | 20 | 54.113 | −15.247 | −15.058 | 1.00 | 93.90 | E |
| ATOM | 14576 | CA | GLN | E | 20 | 54.793 | −14.023 | −14.648 | 1.00 | 89.47 | E |
| ATOM | 14577 | CB | GLN | E | 20 | 55.892 | −14.356 | −13.638 | 1.00 | 101.79 | E |
| ATOM | 14578 | CG | GLN | E | 20 | 56.856 | −15.406 | −14.126 | 1.00 | 102.77 | E |
| ATOM | 14579 | CD | GLN | E | 20 | 57.883 | −15.775 | −13.081 | 1.00 | 102.53 | E |
| ATOM | 14580 | OE1 | GLN | E | 20 | 57.534 | −16.232 | −11.996 | 1.00 | 102.18 | E |
| ATOM | 14581 | NE2 | GLN | E | 20 | 59.160 | −15.578 | −13.401 | 1.00 | 101.98 | E |
| ATOM | 14582 | C | GLN | E | 20 | 55.382 | −13.252 | −15.825 | 1.00 | 84.21 | E |
| ATOM | 14583 | O | GLN | E | 20 | 55.416 | −13.748 | −16.947 | 1.00 | 82.92 | E |
| ATOM | 14584 | N | LEU | E | 21 | 55.839 | −12.032 | −15.559 | 1.00 | 58.38 | E |
| ATOM | 14585 | CA | LEU | E | 21 | 56.420 | −11.185 | −16.586 | 1.00 | 54.84 | E |
| ATOM | 14586 | CB | LEU | E | 21 | 55.674 | −9.851 | −16.646 | 1.00 | 60.09 | E |
| ATOM | 14587 | CG | LEU | E | 21 | 56.192 | −8.789 | −17.617 | 1.00 | 58.87 | E |
| ATOM | 14588 | CD1 | LEU | E | 21 | 56.422 | −9.384 | −18.993 | 1.00 | 56.17 | E |
| ATOM | 14589 | CD2 | LEU | E | 21 | 55.177 | −7.660 | −17.696 | 1.00 | 60.91 | E |
| ATOM | 14590 | C | LEU | E | 21 | 57.891 | −10.942 | −16.298 | 1.00 | 53.45 | E |
| ATOM | 14591 | O | LEU | E | 21 | 58.247 | −9.998 | −15.604 | 1.00 | 48.77 | E |
| ATOM | 14592 | N | GLN | E | 22 | 58.745 | −11.796 | −16.840 | 1.00 | 52.26 | E |
| ATOM | 14593 | CA | GLN | E | 22 | 60.184 | −11.684 | −16.634 | 1.00 | 56.86 | E |
| ATOM | 14594 | CB | GLN | E | 22 | 60.724 | −13.055 | −16.239 | 1.00 | 82.01 | E |
| ATOM | 14595 | CG | GLN | E | 22 | 62.228 | −13.162 | −16.153 | 1.00 | 88.78 | E |
| ATOM | 14596 | CD | GLN | E | 22 | 62.685 | −14.606 | −16.056 | 1.00 | 95.00 | E |
| ATOM | 14597 | OE1 | GLN | E | 22 | 63.882 | −14.897 | −16.112 | 1.00 | 100.23 | E |
| ATOM | 14598 | NE2 | GLN | E | 22 | 61.730 | −15.520 | −15.911 | 1.00 | 95.76 | E |
| ATOM | 14599 | C | GLN | E | 22 | 60.864 | −11.185 | −17.910 | 1.00 | 54.25 | E |
| ATOM | 14600 | O | GLN | E | 22 | 60.692 | −11.766 | −18.975 | 1.00 | 53.20 | E |
| ATOM | 14601 | N | TRP | E | 23 | 61.643 | −10.114 | −17.805 | 1.00 | 60.66 | E |
| ATOM | 14602 | CA | TRP | E | 23 | 62.313 | −9.544 | −18.974 | 1.00 | 54.54 | E |
| ATOM | 14603 | CB | TRP | E | 23 | 62.521 | −8.033 | −18.797 | 1.00 | 53.58 | E |
| ATOM | 14604 | CG | TRP | E | 23 | 61.284 | −7.261 | −18.492 | 1.00 | 54.10 | E |
| ATOM | 14605 | CD2 | TRP | E | 23 | 60.183 | −7.010 | −19.377 | 1.00 | 52.21 | E |
| ATOM | 14606 | CE2 | TRP | E | 23 | 59.245 | −6.229 | −18.662 | 1.00 | 53.83 | E |
| ATOM | 14607 | CE3 | TRP | E | 23 | 59.896 | −7.368 | −20.701 | 1.00 | 51.90 | E |
| ATOM | 14608 | CD1 | TRP | E | 23 | 60.980 | −6.645 | −17.318 | 1.00 | 53.57 | E |
| ATOM | 14609 | NE1 | TRP | E | 23 | 59.758 | −6.020 | −17.409 | 1.00 | 52.66 | E |
| ATOM | 14610 | CZ2 | TRP | E | 23 | 58.040 | −5.798 | −19.226 | 1.00 | 55.89 | E |
| ATOM | 14611 | CZ3 | TRP | E | 23 | 58.695 | −6.940 | −21.266 | 1.00 | 56.34 | E |
| ATOM | 14612 | CH2 | TRP | E | 23 | 57.782 | −6.161 | −20.525 | 1.00 | 54.15 | E |
| ATOM | 14613 | C | TRP | E | 23 | 63.670 | −10.155 | −19.274 | 1.00 | 55.04 | E |
| ATOM | 14614 | O | TRP | E | 23 | 64.463 | −10.382 | −18.362 | 1.00 | 56.32 | E |
| ATOM | 14615 | N | LEU | E | 24 | 63.938 | −10.416 | −20.553 | 1.00 | 44.13 | E |
| ATOM | 14616 | CA | LEU | E | 24 | 65.241 | −10.940 | −20.953 | 1.00 | 41.82 | E |
| ATOM | 14617 | CB | LEU | E | 24 | 65.192 | −12.404 | −21.393 | 1.00 | 80.57 | E |
| ATOM | 14618 | CG | LEU | E | 24 | 64.006 | −13.294 | −21.065 | 1.00 | 81.30 | E |
| ATOM | 14619 | CD1 | LEU | E | 24 | 62.843 | −12.882 | −21.934 | 1.00 | 82.92 | E |
| ATOM | 14620 | CD2 | LEU | E | 24 | 64.365 | −14.739 | −21.335 | 1.00 | 76.41 | E |
| ATOM | 14621 | C | LEU | E | 24 | 65.767 | −10.118 | −22.105 | 1.00 | 48.68 | E |
| ATOM | 14622 | O | LEU | E | 24 | 65.008 | −9.453 | −22.814 | 1.00 | 45.66 | E |
| ATOM | 14623 | N | ASN | E | 25 | 67.079 | −10.176 | −22.278 | 1.00 | 39.02 | E |
| ATOM | 14624 | CA | ASN | E | 25 | 67.748 | −9.461 | −23.341 | 1.00 | 40.46 | E |
| ATOM | 14625 | CB | ASN | E | 25 | 68.725 | −8.466 | −22.735 | 1.00 | 54.07 | E |
| ATOM | 14626 | CG | ASN | E | 25 | 69.946 | −9.153 | −22.142 | 1.00 | 55.64 | E |
| ATOM | 14627 | OD1 | ASN | E | 25 | 71.070 | −8.982 | −22.619 | 1.00 | 59.63 | E |
| ATOM | 14628 | ND2 | ASN | E | 25 | 69.724 | −9.952 | −21.109 | 1.00 | 52.04 | E |
| ATOM | 14629 | C | ASN | E | 25 | 68.528 | −10.442 | −24.227 | 1.00 | 40.99 | E |
| ATOM | 14630 | O | ASN | E | 25 | 69.111 | −10.045 | −25.232 | 1.00 | 43.35 | E |
| ATOM | 14631 | N | ARG | E | 26 | 68.548 | −11.715 | −23.847 | 1.00 | 53.10 | E |
| ATOM | 14632 | CA | ARG | E | 26 | 69.291 | −12.720 | −24.602 | 1.00 | 54.38 | E |
| ATOM | 14633 | CB | ARG | E | 26 | 69.776 | −13.823 | −23.652 | 1.00 | 79.50 | E |
| ATOM | 14634 | CG | ARG | E | 26 | 68.677 | −14.471 | −22.830 | 1.00 | 80.41 | E |
| ATOM | 14635 | CD | ARG | E | 26 | 69.187 | −15.739 | −22.174 | 1.00 | 84.47 | E |
| ATOM | 14636 | NE | ARG | E | 26 | 68.156 | −16.405 | −21.388 | 1.00 | 85.61 | E |
| ATOM | 14637 | CZ | ARG | E | 26 | 67.837 | −16.060 | −20.149 | 1.00 | 86.17 | E |
| ATOM | 14638 | NH1 | ARG | E | 26 | 68.480 | −15.057 | −19.561 | 1.00 | 86.65 | E |
| ATOM | 14639 | NH2 | ARG | E | 26 | 66.873 | −16.714 | −19.503 | 1.00 | 85.20 | E |
| ATOM | 14640 | C | ARG | E | 26 | 68.512 | −13.348 | −25.760 | 1.00 | 52.44 | E |
| ATOM | 14641 | O | ARG | E | 26 | 68.524 | −14.567 | −25.929 | 1.00 | 52.15 | E |
| ATOM | 14642 | N | ARG | E | 27 | 67.849 | −12.520 | −26.564 | 1.00 | 45.51 | E |
| ATOM | 14643 | CA | ARG | E | 27 | 67.069 | −13.029 | −27.679 | 1.00 | 46.44 | E |
| ATOM | 14644 | CB | ARG | E | 27 | 65.579 | −12.975 | −27.356 | 1.00 | 42.85 | E |
| ATOM | 14645 | CG | ARG | E | 27 | 65.179 | −13.924 | −26.259 | 1.00 | 44.27 | E |
| ATOM | 14646 | CD | ARG | E | 27 | 65.686 | −15.321 | −26.563 | 1.00 | 46.64 | E |
| ATOM | 14647 | NE | ARG | E | 27 | 65.435 | −16.272 | −25.482 | 1.00 | 50.03 | E |
| ATOM | 14648 | CZ | ARG | E | 27 | 64.221 | −16.636 | −25.088 | 1.00 | 53.64 | E |
| ATOM | 14649 | NH1 | ARG | E | 27 | 64.082 | −17.516 | −24.097 | 1.00 | 54.51 | E |
| ATOM | 14650 | NH2 | ARG | E | 27 | 63.145 | −16.113 | −25.690 | 1.00 | 51.87 | E |
| ATOM | 14651 | C | ARG | E | 27 | 67.329 | −12.311 | −28.981 | 1.00 | 46.90 | E |
| ATOM | 14652 | O | ARG | E | 27 | 68.072 | −11.325 | −29.028 | 1.00 | 47.04 | E |
| ATOM | 14653 | N | ALA | E | 28 | 66.692 | −12.838 | −30.026 | 1.00 | 85.29 | E |
| ATOM | 14654 | CA | ALA | E | 28 | 66.772 | −12.352 | −31.398 | 1.00 | 84.19 | E |
| ATOM | 14655 | CB | ALA | E | 28 | 65.491 | −12.699 | −32.137 | 1.00 | 90.55 | E |
| ATOM | 14656 | C | ALA | E | 28 | 67.056 | −10.879 | −31.563 | 1.00 | 83.85 | E |
| ATOM | 14657 | O | ALA | E | 28 | 68.205 | −10.474 | −31.727 | 1.00 | 84.87 | E |
| ATOM | 14658 | N | ASN | E | 29 | 66.012 | −10.066 | −31.521 | 1.00 | 43.60 | E |
| ATOM | 14659 | CA | ASN | E | 29 | 66.218 | −8.647 | −31.729 | 1.00 | 42.77 | E |
| ATOM | 14660 | CB | ASN | E | 29 | 65.294 | −8.164 | −32.850 | 1.00 | 43.99 | E |
| ATOM | 14661 | CG | ASN | E | 29 | 65.599 | −8.832 | −34.187 | 1.00 | 44.05 | E |
| ATOM | 14662 | OD1 | ASN | E | 29 | 66.531 | −8.443 | −34.893 | 1.00 | 49.91 | E |
| ATOM | 14663 | ND2 | ASN | E | 29 | 64.821 | −9.852 | −34.531 | 1.00 | 41.06 | E |
| ATOM | 14664 | C | ASN | E | 29 | 65.997 | −7.845 | −30.458 | 1.00 | 41.11 | E |
| ATOM | 14665 | O | ASN | E | 29 | 65.122 | −6.969 | −30.393 | 1.00 | 38.83 | E |
| ATOM | 14666 | N | ALA | E | 30 | 66.803 | −8.142 | −29.445 | 1.00 | 34.19 | E |
| ATOM | 14667 | CA | ALA | E | 30 | 66.711 | −7.453 | −28.169 | 1.00 | 29.01 | E |
| ATOM | 14668 | CB | ALA | E | 30 | 66.468 | −8.453 | −27.047 | 1.00 | 33.75 | E |
| ATOM | 14669 | C | ALA | E | 30 | 67.995 | −6.672 | −27.920 | 1.00 | 30.41 | E |
| ATOM | 14670 | O | ALA | E | 30 | 68.992 | −6.866 | −28.628 | 1.00 | 34.56 | E |
| ATOM | 14671 | N | LEU | E | 31 | 67.963 | −5.785 | −26.924 | 1.00 | 43.38 | E |
| ATOM | 14672 | CA | LEU | E | 31 | 69.125 | −4.971 | −26.608 | 1.00 | 42.85 | E |
| ATOM | 14673 | CB | LEU | E | 31 | 69.038 | −3.631 | −27.320 | 1.00 | 38.41 | E |
| ATOM | 14674 | CG | LEU | E | 31 | 69.210 | −3.545 | −28.826 | 1.00 | 41.29 | E |
| ATOM | 14675 | CD1 | LEU | E | 31 | 68.750 | −2.179 | −29.313 | 1.00 | 38.91 | E |
| ATOM | 14676 | CD2 | LEU | E | 31 | 70.657 | −3.783 | −29.172 | 1.00 | 40.76 | E |
| ATOM | 14677 | C | LEU | E | 31 | 69.292 | −4.695 | −25.130 | 1.00 | 42.11 | E |
| ATOM | 14678 | O | LEU | E | 31 | 68.309 | −4.621 | −24.392 | 1.00 | 42.92 | E |
| ATOM | 14679 | N | LEU | E | 32 | 70.553 | −4.537 | −24.726 | 1.00 | 40.42 | E |
| ATOM | 14680 | CA | LEU | E | 32 | 70.945 | −4.220 | −23.358 | 1.00 | 40.21 | E |
| ATOM | 14681 | CB | LEU | E | 32 | 70.829 | −5.434 | −22.443 | 1.00 | 46.34 | E |
| ATOM | 14682 | CG | LEU | E | 32 | 71.127 | −5.069 | −20.983 | 1.00 | 44.92 | E |
| ATOM | 14683 | CD1 | LEU | E | 32 | 70.044 | −4.124 | −20.472 | 1.00 | 43.90 | E |
| ATOM | 14684 | CD2 | LEU | E | 32 | 71.179 | −6.331 | −20.114 | 1.00 | 45.24 | E |
| ATOM | 14685 | C | LEU | E | 32 | 72.396 | −3.793 | −23.467 | 1.00 | 39.85 | E |
| ATOM | 14686 | O | LEU | E | 32 | 73.302 | −4.581 | −23.209 | 1.00 | 45.72 | E |
| ATOM | 14687 | N | ALA | E | 33 | 72.619 | −2.538 | −23.844 | 1.00 | 45.34 | E |
| ATOM | 14688 | CA | ALA | E | 33 | 73.979 | −2.068 | −24.033 | 1.00 | 45.86 | E |
| ATOM | 14689 | CB | ALA | E | 33 | 74.315 | −2.114 | −25.505 | 1.00 | 87.59 | E |
| ATOM | 14690 | C | ALA | E | 33 | 74.356 | −0.706 | −23.490 | 1.00 | 47.65 | E |
| ATOM | 14691 | O | ALA | E | 33 | 73.529 | 0.055 | −23.004 | 1.00 | 51.83 | E |
| ATOM | 14692 | N | ASN | E | 34 | 75.649 | −0.429 | −23.597 | 1.00 | 51.43 | E |
| ATOM | 14693 | CA | ASN | E | 34 | 76.247 | 0.820 | −23.174 | 1.00 | 48.62 | E |
| ATOM | 14694 | CB | ASN | E | 34 | 75.871 | 1.902 | −24.162 | 1.00 | 60.79 | E |
| ATOM | 14695 | CG | ASN | E | 34 | 76.342 | 1.577 | −25.559 | 1.00 | 64.71 | E |
| ATOM | 14696 | OD1 | ASN | E | 34 | 77.511 | 1.261 | −25.767 | 1.00 | 67.19 | E |
| ATOM | 14697 | ND2 | ASN | E | 34 | 75.439 | 1.652 | −26.525 | 1.00 | 60.12 | E |
| ATOM | 14698 | C | ASN | E | 34 | 75.950 | 1.276 | −21.769 | 1.00 | 49.37 | E |
| ATOM | 14699 | O | ASN | E | 34 | 75.716 | 2.450 | −21.534 | 1.00 | 51.91 | E |
| ATOM | 14700 | N | GLY | E | 35 | 75.950 | 0.351 | −20.824 | 1.00 | 44.24 | E |
| ATOM | 14701 | CA | GLY | E | 35 | 75.725 | 0.765 | −19.460 | 1.00 | 38.83 | E |
| ATOM | 14702 | C | GLY | E | 35 | 74.437 | 0.393 | −18.776 | 1.00 | 39.18 | E |
| ATOM | 14703 | O | GLY | E | 35 | 74.438 | 0.139 | −17.577 | 1.00 | 40.67 | E |
| ATOM | 14704 | N | VAL | E | 36 | 73.327 | 0.358 | −19.486 | 1.00 | 40.82 | E |
| ATOM | 14705 | CA | VAL | E | 36 | 72.109 | 0.025 | −18.775 | 1.00 | 42.15 | E |
| ATOM | 14706 | CB | VAL | E | 36 | 70.824 | 0.376 | −19.609 | 1.00 | 36.18 | E |
| ATOM | 14707 | CG1 | VAL | E | 36 | 71.208 | 0.775 | −21.010 | 1.00 | 34.21 | E |
| ATOM | 14708 | CG2 | VAL | E | 36 | 69.839 | −0.767 | −19.610 | 1.00 | 36.58 | E |
| ATOM | 14709 | C | VAL | E | 36 | 72.159 | −1.433 | −18.355 | 1.00 | 46.30 | E |
| ATOM | 14710 | O | VAL | E | 36 | 72.648 | −2.299 | −19.091 | 1.00 | 47.26 | E |
| ATOM | 14711 | N | GLU | E | 37 | 71.682 | −1.695 | −17.146 | 1.00 | 43.75 | E |
| ATOM | 14712 | CA | GLU | E | 37 | 71.686 | −3.041 | −16.613 | 1.00 | 46.35 | E |
| ATOM | 14713 | CB | GLU | E | 37 | 72.482 | −3.065 | −15.308 | 1.00 | 58.26 | E |
| ATOM | 14714 | CG | GLU | E | 37 | 73.709 | −2.181 | −15.346 | 1.00 | 65.29 | E |
| ATOM | 14715 | CD | GLU | E | 37 | 74.289 | −1.904 | −13.967 | 1.00 | 68.26 | E |
| ATOM | 14716 | OE1 | GLU | E | 37 | 73.518 | −1.899 | −12.973 | 1.00 | 71.69 | E |
| ATOM | 14717 | OE2 | GLU | E | 37 | 75.515 | −1.669 | −13.882 | 1.00 | 68.82 | E |
| ATOM | 14718 | C | GLU | E | 37 | 70.266 | −3.524 | −16.358 | 1.00 | 45.24 | E |
| ATOM | 14719 | O | GLU | E | 37 | 69.346 | −2.733 | −16.168 | 1.00 | 47.70 | E |
| ATOM | 14720 | N | LEU | E | 38 | 70.094 | −4.837 | −16.379 | 1.00 | 57.76 | E |
| ATOM | 14721 | CA | LEU | E | 38 | 68.803 | −5.427 | −16.110 | 1.00 | 59.80 | E |
| ATOM | 14722 | CB | LEU | E | 38 | 68.458 | −6.439 | −17.192 | 1.00 | 46.50 | E |
| ATOM | 14723 | CG | LEU | E | 38 | 67.080 | −7.080 | −17.073 | 1.00 | 44.41 | E |
| ATOM | 14724 | CD1 | LEU | E | 38 | 66.036 | −6.006 | −16.835 | 1.00 | 42.79 | E |
| ATOM | 14725 | CD2 | LEU | E | 38 | 66.768 | −7.892 | −18.343 | 1.00 | 41.28 | E |
| ATOM | 14726 | C | LEU | E | 38 | 69.036 | −6.099 | −14.770 | 1.00 | 60.07 | E |
| ATOM | 14727 | O | LEU | E | 38 | 69.678 | −7.144 | −14.692 | 1.00 | 53.68 | E |
| ATOM | 14728 | N | ARG | E | 39 | 68.535 | −5.473 | −13.712 | 1.00 | 56.58 | E |
| ATOM | 14729 | CA | ARG | E | 39 | 68.728 | −5.961 | −12.354 | 1.00 | 63.11 | E |
| ATOM | 14730 | CB | ARG | E | 39 | 69.598 | −4.951 | −11.613 | 1.00 | 53.01 | E |
| ATOM | 14731 | CG | ARG | E | 39 | 70.110 | −5.395 | −10.281 | 1.00 | 60.71 | E |
| ATOM | 14732 | CD | ARG | E | 39 | 71.166 | −4.425 | −9.791 | 1.00 | 69.32 | E |
| ATOM | 14733 | NE | ARG | E | 39 | 71.898 | −4.964 | −8.649 | 1.00 | 75.85 | E |
| ATOM | 14734 | CZ | ARG | E | 39 | 73.037 | −4.454 | −8.181 | 1.00 | 76.65 | E |
| ATOM | 14735 | NH1 | ARG | E | 39 | 73.577 | −3.383 | −8.760 | 1.00 | 75.13 | E |
| ATOM | 14736 | NH2 | ARG | E | 39 | 73.647 | −5.016 | −7.140 | 1.00 | 78.95 | E |
| ATOM | 14737 | C | ARG | E | 39 | 67.395 | −6.145 | −11.642 | 1.00 | 65.16 | E |
| ATOM | 14738 | O | ARG | E | 39 | 66.551 | −5.253 | −11.657 | 1.00 | 68.39 | E |
| ATOM | 14739 | N | ASP | E | 40 | 67.205 | −7.304 | −11.024 | 1.00 | 63.60 | E |
| ATOM | 14740 | CA | ASP | E | 40 | 65.967 | −7.595 | −10.308 | 1.00 | 61.55 | E |
| ATOM | 14741 | CB | ASP | E | 40 | 65.913 | −6.794 | −8.998 | 1.00 | 120.11 | E |
| ATOM | 14742 | CG | ASP | E | 40 | 67.131 | −7.027 | −8.114 | 1.00 | 122.25 | E |
| ATOM | 14743 | OD1 | ASP | E | 40 | 67.438 | −8.204 | −7.829 | 1.00 | 119.13 | E |
| ATOM | 14744 | OD2 | ASP | E | 40 | 67.777 | −6.036 | −7.700 | 1.00 | 122.37 | E |
| ATOM | 14745 | C | ASP | E | 40 | 64.738 | −7.275 | −11.172 | 1.00 | 57.68 | E |
| ATOM | 14746 | O | ASP | E | 40 | 63.720 | −6.784 | −10.684 | 1.00 | 56.91 | E |
| ATOM | 14747 | N | ASN | E | 41 | 64.850 | −7.550 | −12.466 | 1.00 | 63.50 | E |
| ATOM | 14748 | CA | ASN | E | 41 | 63.764 | −7.320 | −13.420 | 1.00 | 59.25 | E |
| ATOM | 14749 | CB | ASN | E | 41 | 62.491 | −8.022 | −12.951 | 1.00 | 53.81 | E |
| ATOM | 14750 | CG | ASN | E | 41 | 61.592 | −8.402 | −14.108 | 1.00 | 48.50 | E |
| ATOM | 14751 | OD1 | ASN | E | 41 | 62.033 | −9.083 | −15.045 | 1.00 | 49.84 | E |
| ATOM | 14752 | ND2 | ASN | E | 41 | 60.331 | −7.968 | −14.060 | 1.00 | 44.60 | E |
| ATOM | 14753 | C | ASN | E | 41 | 63.449 | −5.848 | −13.706 | 1.00 | 62.56 | E |
| ATOM | 14754 | O | ASN | E | 41 | 62.332 | −5.501 | −14.100 | 1.00 | 64.07 | E |
| ATOM | 14755 | N | GLN | E | 42 | 64.440 | −4.988 | −13.514 | 1.00 | 57.70 | E |
| ATOM | 14756 | CA | GLN | E | 42 | 64.258 | −3.566 | −13.756 | 1.00 | 55.62 | E |
| ATOM | 14757 | CB | GLN | E | 42 | 64.178 | −2.799 | −12.436 | 1.00 | 51.87 | E |
| ATOM | 14758 | CG | GLN | E | 42 | 63.231 | −3.420 | −11.433 | 1.00 | 53.96 | E |
| ATOM | 14759 | CD | GLN | E | 42 | 63.203 | −2.685 | −10.107 | 1.00 | 53.42 | E |
| ATOM | 14760 | OE1 | GLN | E | 42 | 64.246 | −2.388 | −9.515 | 1.00 | 52.85 | E |
| ATOM | 14761 | NE2 | GLN | E | 42 | 62.000 | −2.399 | −9.627 | 1.00 | 55.18 | E |
| ATOM | 14762 | C | GLN | E | 42 | 65.448 | −3.070 | −14.542 | 1.00 | 54.51 | E |
| ATOM | 14763 | O | GLN | E | 42 | 66.551 | −3.603 | −14.418 | 1.00 | 54.85 | E |
| ATOM | 14764 | N | LEU | E | 43 | 65.222 | −2.058 | −15.366 | 1.00 | 45.09 | E |
| ATOM | 14765 | CA | LEU | E | 43 | 66.308 | −1.489 | −16.144 | 1.00 | 43.67 | E |
| ATOM | 14766 | CB | LEU | E | 43 | 65.765 | −0.892 | −17.440 | 1.00 | 40.65 | E |
| ATOM | 14767 | CG | LEU | E | 43 | 65.157 | −1.921 | −18.393 | 1.00 | 46.81 | E |
| ATOM | 14768 | CD1 | LEU | E | 43 | 64.474 | −1.207 | −19.540 | 1.00 | 46.90 | E |
| ATOM | 14769 | CD2 | LEU | E | 43 | 66.238 | −2.842 | −18.897 | 1.00 | 43.20 | E |
| ATOM | 14770 | C | LEU | E | 43 | 66.939 | −0.410 | −15.280 | 1.00 | 45.69 | E |
| ATOM | 14771 | O | LEU | E | 43 | 66.226 | 0.334 | −14.605 | 1.00 | 42.31 | E |
| ATOM | 14772 | N | VAL | E | 44 | 68.266 | −0.330 | −15.284 | 1.00 | 44.24 | E |
| ATOM | 14773 | CA | VAL | E | 44 | 68.951 | 0.659 | −14.474 | 1.00 | 43.92 | E |
| ATOM | 14774 | CB | VAL | E | 44 | 69.867 | −0.006 | −13.447 | 1.00 | 43.03 | E |
| ATOM | 14775 | CG1 | VAL | E | 44 | 70.423 | 1.036 | −12.504 | 1.00 | 44.22 | E |
| ATOM | 14776 | CG2 | VAL | E | 44 | 69.087 | −1.044 | −12.673 | 1.00 | 37.64 | E |
| ATOM | 14777 | C | VAL | E | 44 | 69.767 | 1.599 | −15.332 | 1.00 | 42.93 | E |
| ATOM | 14778 | O | VAL | E | 44 | 70.708 | 1.178 | −16.017 | 1.00 | 43.97 | E |
| ATOM | 14779 | N | VAL | E | 45 | 69.400 | 2.880 | −15.275 | 1.00 | 46.96 | E |
| ATOM | 14780 | CA | VAL | E | 45 | 70.061 | 3.926 | −16.040 | 1.00 | 52.70 | E |
| ATOM | 14781 | CB | VAL | E | 45 | 69.233 | 5.208 | −15.964 | 1.00 | 44.44 | E |
| ATOM | 14782 | CG1 | VAL | E | 45 | 70.029 | 6.378 | −16.493 | 1.00 | 47.01 | E |
| ATOM | 14783 | CG2 | VAL | E | 45 | 67.966 | 5.036 | −16.773 | 1.00 | 47.84 | E |
| ATOM | 14784 | C | VAL | E | 45 | 71.495 | 4.194 | −15.566 | 1.00 | 54.49 | E |
| ATOM | 14785 | O | VAL | E | 45 | 71.754 | 4.342 | −14.375 | 1.00 | 53.94 | E |
| ATOM | 14786 | N | PRO | E | 46 | 72.449 | 4.258 | −16.503 | 1.00 | 48.65 | E |
| ATOM | 14787 | CD | PRO | E | 46 | 72.298 | 3.823 | −17.905 | 1.00 | 47.27 | E |
| ATOM | 14788 | CA | PRO | E | 46 | 73.860 | 4.505 | −16.184 | 1.00 | 47.50 | E |
| ATOM | 14789 | CB | PRO | E | 46 | 74.585 | 3.728 | −17.266 | 1.00 | 46.63 | E |
| ATOM | 14790 | CG | PRO | E | 46 | 73.719 | 3.992 | −18.458 | 1.00 | 47.33 | E |
| ATOM | 14791 | C | PRO | E | 46 | 74.332 | 5.952 | −16.155 | 1.00 | 50.94 | E |
| ATOM | 14792 | O | PRO | E | 46 | 75.484 | 6.209 | −15.819 | 1.00 | 53.40 | E |
| ATOM | 14793 | N | SER | E | 47 | 73.474 | 6.900 | −16.521 | 1.00 | 65.00 | E |
| ATOM | 14794 | CA | SER | E | 47 | 73.899 | 8.296 | −16.519 | 1.00 | 62.12 | E |
| ATOM | 14795 | CB | SER | E | 47 | 74.820 | 8.540 | −17.703 | 1.00 | 51.36 | E |
| ATOM | 14796 | OG | SER | E | 47 | 74.141 | 8.249 | −18.907 | 1.00 | 52.57 | E |
| ATOM | 14797 | C | SER | E | 47 | 72.757 | 9.298 | −16.562 | 1.00 | 58.81 | E |
| ATOM | 14798 | O | SER | E | 47 | 71.643 | 8.961 | −16.970 | 1.00 | 55.95 | E |
| ATOM | 14799 | N | GLU | E | 48 | 73.049 | 10.529 | −16.129 | 1.00 | 64.98 | E |
| ATOM | 14800 | CA | GLU | E | 48 | 72.080 | 11.631 | −16.132 | 1.00 | 69.47 | E |
| ATOM | 14801 | CB | GLU | E | 48 | 72.742 | 12.944 | −15.707 | 1.00 | 168.57 | E |
| ATOM | 14802 | CG | GLU | E | 48 | 73.444 | 12.931 | −14.372 | 1.00 | 173.31 | E |
| ATOM | 14803 | CD | GLU | E | 48 | 72.508 | 13.138 | −13.199 | 1.00 | 177.66 | E |
| ATOM | 14804 | OE1 | GLU | E | 48 | 71.684 | 12.241 | −12.923 | 1.00 | 179.40 | E |
| ATOM | 14805 | OE2 | GLU | E | 48 | 72.598 | 14.206 | −12.554 | 1.00 | 175.58 | E |
| ATOM | 14806 | C | GLU | E | 48 | 71.629 | 11.793 | −17.579 | 1.00 | 68.06 | E |
| ATOM | 14807 | O | GLU | E | 48 | 72.394 | 11.518 | −18.508 | 1.00 | 73.85 | E |
| ATOM | 14808 | N | GLY | E | 49 | 70.391 | 12.228 | −17.781 | 1.00 | 39.38 | E |
| ATOM | 14809 | CA | GLY | E | 49 | 69.940 | 12.430 | −19.142 | 1.00 | 37.04 | E |
| ATOM | 14810 | C | GLY | E | 49 | 68.464 | 12.276 | −19.428 | 1.00 | 39.05 | E |
| ATOM | 14811 | O | GLY | E | 49 | 67.676 | 11.869 | −18.571 | 1.00 | 41.89 | E |
| ATOM | 14812 | N | LEU | E | 50 | 68.120 | 12.626 | −20.663 | 1.00 | 47.62 | E |
| ATOM | 14813 | CA | LEU | E | 50 | 66.769 | 12.547 | −21.184 | 1.00 | 48.82 | E |
| ATOM | 14814 | CB | LEU | E | 50 | 66.592 | 13.618 | −22.260 | 1.00 | 38.61 | E |
| ATOM | 14815 | CG | LEU | E | 50 | 65.628 | 14.791 | −22.018 | 1.00 | 39.74 | E |
| ATOM | 14816 | CD1 | LEU | E | 50 | 65.523 | 15.123 | −20.545 | 1.00 | 36.55 | E |
| ATOM | 14817 | CD2 | LEU | E | 50 | 66.098 | 16.001 | −22.807 | 1.00 | 40.13 | E |
| ATOM | 14818 | C | LEU | E | 50 | 66.644 | 11.153 | −21.789 | 1.00 | 45.20 | E |
| ATOM | 14819 | O | LEU | E | 50 | 67.503 | 10.726 | −22.567 | 1.00 | 43.92 | E |
| ATOM | 14820 | N | TYR | E | 51 | 65.594 | 10.429 | −21.419 | 1.00 | 42.87 | E |
| ATOM | 14821 | CA | TYR | E | 51 | 65.393 | 9.076 | −21.945 | 1.00 | 41.52 | E |
| ATOM | 14822 | CB | TYR | E | 51 | 65.616 | 8.010 | −20.862 | 1.00 | 40.78 | E |
| ATOM | 14823 | CG | TYR | E | 51 | 67.042 | 7.783 | −20.447 | 1.00 | 40.88 | E |
| ATOM | 14824 | CD1 | TYR | E | 51 | 67.714 | 8.711 | −19.655 | 1.00 | 41.74 | E |
| ATOM | 14825 | CE1 | TYR | E | 51 | 69.026 | 8.499 | −19.257 | 1.00 | 41.30 | E |
| ATOM | 14826 | CD2 | TYR | E | 51 | 67.720 | 6.631 | −20.837 | 1.00 | 42.44 | E |
| ATOM | 14827 | CE2 | TYR | E | 51 | 69.034 | 6.406 | −20.448 | 1.00 | 40.71 | E |
| ATOM | 14828 | CZ | TYR | E | 51 | 69.684 | 7.343 | −19.656 | 1.00 | 40.41 | E |
| ATOM | 14829 | OH | TYR | E | 51 | 70.995 | 7.129 | −19.258 | 1.00 | 40.64 | E |
| ATOM | 14830 | C | TYR | E | 51 | 64.003 | 8.834 | −22.506 | 1.00 | 41.70 | E |
| ATOM | 14831 | O | TYR | E | 51 | 63.002 | 9.312 | −21.968 | 1.00 | 42.59 | E |
| ATOM | 14832 | N | LEU | E | 52 | 63.947 | 8.076 | −23.590 | 1.00 | 39.99 | E |
| ATOM | 14833 | CA | LEU | E | 52 | 62.670 | 7.719 | −24.174 | 1.00 | 41.64 | E |
| ATOM | 14834 | CB | LEU | E | 52 | 62.765 | 7.632 | −25.705 | 1.00 | 56.53 | E |
| ATOM | 14835 | CG | LEU | E | 52 | 61.560 | 6.986 | −26.399 | 1.00 | 52.42 | E |
| ATOM | 14836 | CD1 | LEU | E | 52 | 60.270 | 7.664 | −25.960 | 1.00 | 54.45 | E |
| ATOM | 14837 | CD2 | LEU | E | 52 | 61.742 | 7.063 | −27.909 | 1.00 | 46.75 | E |
| ATOM | 14838 | C | LEU | E | 52 | 62.392 | 6.350 | −23.555 | 1.00 | 43.17 | E |
| ATOM | 14839 | O | LEU | E | 52 | 63.198 | 5.440 | −23.680 | 1.00 | 43.74 | E |
| ATOM | 14840 | N | ILE | E | 53 | 61.275 | 6.229 | −22.849 | 1.00 | 42.46 | E |
| ATOM | 14841 | CA | ILE | E | 53 | 60.907 | 4.981 | −22.198 | 1.00 | 39.06 | E |
| ATOM | 14842 | CB | ILE | E | 53 | 60.643 | 5.201 | −20.710 | 1.00 | 45.82 | E |
| ATOM | 14843 | CG2 | ILE | E | 53 | 60.320 | 3.892 | −20.050 | 1.00 | 46.46 | E |
| ATOM | 14844 | CG1 | ILE | E | 53 | 61.857 | 5.839 | −20.051 | 1.00 | 44.71 | E |
| ATOM | 14845 | CD1 | ILE | E | 53 | 61.640 | 6.120 | −18.590 | 1.00 | 40.53 | E |
| ATOM | 14846 | C | ILE | E | 53 | 59.625 | 4.477 | −22.821 | 1.00 | 39.12 | E |
| ATOM | 14847 | O | ILE | E | 53 | 58.715 | 5.264 | −23.093 | 1.00 | 42.20 | E |
| ATOM | 14848 | N | TYR | E | 54 | 59.531 | 3.173 | −23.039 | 1.00 | 42.64 | E |
| ATOM | 14849 | CA | TYR | E | 54 | 58.325 | 2.616 | −23.655 | 1.00 | 39.77 | E |
| ATOM | 14850 | CB | TYR | E | 54 | 58.380 | 2.792 | −25.174 | 1.00 | 33.95 | E |
| ATOM | 14851 | CG | TYR | E | 54 | 59.501 | 2.011 | −25.814 | 1.00 | 33.95 | E |
| ATOM | 14852 | CD1 | TYR | E | 54 | 59.321 | 0.691 | −26.207 | 1.00 | 33.95 | E |
| ATOM | 14853 | CE1 | TYR | E | 54 | 60.368 | −0.049 | −26.751 | 1.00 | 33.95 | E |
| ATOM | 14854 | CD2 | TYR | E | 54 | 60.767 | 2.579 | −25.985 | 1.00 | 33.95 | E |
| ATOM | 14855 | CE2 | TYR | E | 54 | 61.825 | 1.846 | −26.530 | 1.00 | 33.95 | E |
| ATOM | 14856 | CZ | TYR | E | 54 | 61.610 | 0.535 | −26.909 | 1.00 | 33.95 | E |
| ATOM | 14857 | OH | TYR | E | 54 | 62.624 | −0.199 | −27.469 | 1.00 | 33.95 | E |
| ATOM | 14858 | C | TYR | E | 54 | 58.138 | 1.149 | −23.327 | 1.00 | 38.36 | E |
| ATOM | 14859 | O | TYR | E | 54 | 59.075 | 0.451 | −22.932 | 1.00 | 37.21 | E |
| ATOM | 14860 | N | SER | E | 55 | 56.916 | 0.678 | −23.503 | 1.00 | 33.38 | E |
| ATOM | 14861 | CA | SER | E | 55 | 56.602 | −0.704 | −23.210 | 1.00 | 33.16 | E |
| ATOM | 14862 | CB | SER | E | 55 | 56.577 | −0.924 | −21.699 | 1.00 | 46.70 | E |
| ATOM | 14863 | OG | SER | E | 55 | 56.071 | −2.206 | −21.384 | 1.00 | 53.26 | E |
| ATOM | 14864 | C | SER | E | 55 | 55.257 | −1.109 | −23.778 | 1.00 | 36.14 | E |
| ATOM | 14865 | O | SER | E | 55 | 54.349 | −0.282 | −23.927 | 1.00 | 33.28 | E |
| ATOM | 14866 | N | GLN | E | 56 | 55.132 | −2.388 | −24.099 | 1.00 | 54.02 | E |
| ATOM | 14867 | CA | GLN | E | 56 | 53.876 | −2.895 | −24.604 | 1.00 | 54.72 | E |
| ATOM | 14868 | CB | GLN | E | 56 | 53.861 | −2.975 | −26.125 | 1.00 | 62.97 | E |
| ATOM | 14869 | CG | GLN | E | 56 | 52.489 | −3.395 | −26.628 | 1.00 | 64.57 | E |
| ATOM | 14870 | CD | GLN | E | 56 | 52.482 | −3.840 | −28.071 | 1.00 | 67.69 | E |
| ATOM | 14871 | OE1 | GLN | E | 56 | 52.857 | −4.970 | −28.385 | 1.00 | 67.22 | E |
| ATOM | 14872 | NE2 | GLN | E | 56 | 52.055 | −2.949 | −28.963 | 1.00 | 70.80 | E |
| ATOM | 14873 | C | GLN | E | 56 | 53.612 | −4.282 | −24.064 | 1.00 | 57.20 | E |
| ATOM | 14874 | O | GLN | E | 56 | 54.514 | −5.129 | −24.009 | 1.00 | 55.28 | E |
| ATOM | 14875 | N | VAL | E | 57 | 52.373 | −4.510 | −23.653 | 1.00 | 41.67 | E |
| ATOM | 14876 | CA | VAL | E | 57 | 51.989 | −5.822 | −23.163 | 1.00 | 36.04 | E |
| ATOM | 14877 | CB | VAL | E | 57 | 51.638 | −5.811 | −21.658 | 1.00 | 49.27 | E |
| ATOM | 14878 | CG1 | VAL | E | 57 | 52.820 | −5.328 | −20.853 | 1.00 | 50.47 | E |
| ATOM | 14879 | CG2 | VAL | E | 57 | 50.429 | −4.935 | −21.419 | 1.00 | 46.20 | E |
| ATOM | 14880 | C | VAL | E | 57 | 50.760 | −6.219 | −23.953 | 1.00 | 38.65 | E |
| ATOM | 14881 | O | VAL | E | 57 | 50.009 | −5.358 | −24.409 | 1.00 | 39.76 | E |
| ATOM | 14882 | N | LEU | E | 58 | 50.568 | −7.519 | −24.123 | 1.00 | 73.53 | E |
| ATOM | 14883 | CA | LEU | E | 58 | 49.417 | −8.019 | −24.850 | 1.00 | 71.98 | E |
| ATOM | 14884 | CB | LEU | E | 58 | 49.861 | −8.759 | −26.110 | 1.00 | 69.10 | E |
| ATOM | 14885 | CG | LEU | E | 58 | 48.871 | −8.789 | −27.277 | 1.00 | 71.12 | E |
| ATOM | 14886 | CD1 | LEU | E | 58 | 49.214 | −9.961 | −28.184 | 1.00 | 65.14 | E |
| ATOM | 14887 | CD2 | LEU | E | 58 | 47.452 | −8.916 | −26.766 | 1.00 | 68.90 | E |
| ATOM | 14888 | C | LEU | E | 58 | 48.674 | −8.979 | −23.938 | 1.00 | 74.95 | E |
| ATOM | 14889 | O | LEU | E | 58 | 49.209 | −10.015 | −23.546 | 1.00 | 76.58 | E |
| ATOM | 14890 | N | PHE | E | 59 | 47.445 | −8.630 | −23.585 | 1.00 | 74.98 | E |
| ATOM | 14891 | CA | PHE | E | 59 | 46.647 | −9.489 | −22.723 | 1.00 | 75.88 | E |
| ATOM | 14892 | CB | PHE | E | 59 | 45.781 | −8.664 | −21.769 | 1.00 | 69.18 | E |
| ATOM | 14893 | CG | PHE | E | 59 | 46.560 | −7.883 | −20.757 | 1.00 | 65.67 | E |
| ATOM | 14894 | CD1 | PHE | E | 59 | 46.753 | −6.513 | −20.914 | 1.00 | 64.11 | E |
| ATOM | 14895 | CD2 | PHE | E | 59 | 47.096 | −8.511 | −19.641 | 1.00 | 67.63 | E |
| ATOM | 14896 | CE1 | PHE | E | 59 | 47.463 | −5.780 | −19.974 | 1.00 | 70.03 | E |
| ATOM | 14897 | CE2 | PHE | E | 59 | 47.813 | −7.786 | −18.692 | 1.00 | 70.74 | E |
| ATOM | 14898 | CZ | PHE | E | 59 | 47.995 | −6.415 | −18.860 | 1.00 | 69.34 | E |
| ATOM | 14899 | C | PHE | E | 59 | 45.744 | −10.361 | −23.576 | 1.00 | 79.99 | E |
| ATOM | 14900 | O | PHE | E | 59 | 45.272 | −9.928 | −24.624 | 1.00 | 79.68 | E |
| ATOM | 14901 | N | LYS | E | 60 | 45.511 | −11.588 | −23.121 | 1.00 | 94.25 | E |
| ATOM | 14902 | CA | LYS | E | 60 | 44.652 | −12.529 | −23.827 | 1.00 | 95.60 | E |
| ATOM | 14903 | CB | LYS | E | 60 | 45.480 | −13.457 | −24.713 | 1.00 | 83.56 | E |
| ATOM | 14904 | CG | LYS | E | 60 | 44.662 | −14.527 | −25.414 | 1.00 | 89.75 | E |
| ATOM | 14905 | CD | LYS | E | 60 | 45.566 | −15.463 | −26.200 | 1.00 | 97.96 | E |
| ATOM | 14906 | CE | LYS | E | 60 | 44.777 | −16.570 | −26.882 | 1.00 | 100.71 | E |
| ATOM | 14907 | NZ | LYS | E | 60 | 45.672 | −17.422 | −27.712 | 1.00 | 101.05 | E |
| ATOM | 14908 | C | LYS | E | 60 | 43.884 | −13.358 | −22.816 | 1.00 | 95.11 | E |
| ATOM | 14909 | O | LYS | E | 60 | 44.440 | −13.798 | −21.808 | 1.00 | 97.86 | E |
| ATOM | 14910 | N | GLY | E | 61 | 42.603 | −13.567 | −23.087 | 1.00 | 71.45 | E |
| ATOM | 14911 | CA | GLY | E | 61 | 41.793 | −14.351 | −22.185 | 1.00 | 74.01 | E |
| ATOM | 14912 | C | GLY | E | 61 | 40.801 | −15.205 | −22.939 | 1.00 | 75.71 | E |
| ATOM | 14913 | O | GLY | E | 61 | 40.271 | −14.785 | −23.967 | 1.00 | 78.88 | E |
| ATOM | 14914 | N | GLN | E | 62 | 40.560 | −16.412 | −22.432 | 1.00 | 121.57 | E |
| ATOM | 14915 | CA | GLN | E | 62 | 39.605 | −17.329 | −23.041 | 1.00 | 121.88 | E |
| ATOM | 14916 | CB | GLN | E | 62 | 39.989 | −18.776 | −22.737 | 1.00 | 122.87 | E |
| ATOM | 14917 | CG | GLN | E | 62 | 41.388 | −19.153 | −23.182 | 1.00 | 127.00 | E |
| ATOM | 14918 | CD | GLN | E | 62 | 41.555 | −19.101 | −24.687 | 1.00 | 132.94 | E |
| ATOM | 14919 | OE1 | GLN | E | 62 | 41.377 | −18.054 | −25.310 | 1.00 | 132.54 | E |
| ATOM | 14920 | NE2 | GLN | E | 62 | 41.901 | −20.237 | −25.281 | 1.00 | 133.11 | E |
| ATOM | 14921 | C | GLN | E | 62 | 38.254 | −17.017 | −22.420 | 1.00 | 119.82 | E |
| ATOM | 14922 | O | GLN | E | 62 | 38.095 | −17.111 | −21.204 | 1.00 | 121.33 | E |
| ATOM | 14923 | N | GLY | E | 63 | 37.288 | −16.634 | −23.249 | 1.00 | 110.01 | E |
| ATOM | 14924 | CA | GLY | E | 63 | 35.968 | −16.308 | −22.734 | 1.00 | 108.00 | E |
| ATOM | 14925 | C | GLY | E | 63 | 36.042 | −15.255 | −21.644 | 1.00 | 106.08 | E |
| ATOM | 14926 | O | GLY | E | 63 | 37.089 | −14.645 | −21.441 | 1.00 | 104.12 | E |
| ATOM | 14927 | N | CYS | E | 64 | 34.946 | −15.049 | −20.923 | 1.00 | 137.73 | E |
| ATOM | 14928 | CA | CYS | E | 64 | 34.931 | −14.042 | −19.872 | 1.00 | 141.97 | E |
| ATOM | 14929 | C | CYS | E | 64 | 34.779 | −14.523 | −18.433 | 1.00 | 148.06 | E |
| ATOM | 14930 | O | CYS | E | 64 | 34.032 | −15.459 | −18.147 | 1.00 | 150.97 | E |
| ATOM | 14931 | CB | CYS | E | 64 | 33.852 | −13.005 | −20.168 | 1.00 | 154.70 | E |
| ATOM | 14932 | SG | CYS | E | 64 | 34.413 | −11.783 | −21.382 | 1.00 | 155.91 | E |
| ATOM | 14933 | N | PRO | E | 65 | 35.507 | −13.878 | −17.504 | 1.00 | 134.04 | E |
| ATOM | 14934 | CD | PRO | E | 65 | 36.641 | −12.983 | −17.790 | 1.00 | 162.07 | E |
| ATOM | 14935 | CA | PRO | E | 65 | 35.471 | −14.214 | −16.079 | 1.00 | 136.38 | E |
| ATOM | 14936 | CB | PRO | E | 65 | 36.683 | −13.470 | −15.514 | 1.00 | 164.60 | E |
| ATOM | 14937 | CG | PRO | E | 65 | 37.600 | −13.345 | −16.691 | 1.00 | 164.33 | E |
| ATOM | 14938 | C | PRO | E | 65 | 34.171 | −13.746 | −15.433 | 1.00 | 139.39 | E |
| ATOM | 14939 | O | PRO | E | 65 | 33.131 | −13.642 | −16.086 | 1.00 | 139.66 | E |
| ATOM | 14940 | N | SER | E | 66 | 34.248 | −13.455 | −14.142 | 1.00 | 160.38 | E |
| ATOM | 14941 | CA | SER | E | 66 | 33.097 | −12.995 | −13.391 | 1.00 | 163.98 | E |
| ATOM | 14942 | CB | SER | E | 66 | 33.111 | −13.617 | −11.995 | 1.00 | 162.26 | E |
| ATOM | 14943 | OG | SER | E | 66 | 32.016 | −13.162 | −11.222 | 1.00 | 159.42 | E |
| ATOM | 14944 | C | SER | E | 66 | 33.115 | −11.475 | −13.282 | 1.00 | 164.53 | E |
| ATOM | 14945 | O | SER | E | 66 | 34.053 | −10.821 | −13.743 | 1.00 | 157.95 | E |
| ATOM | 14946 | N | THR | E | 67 | 32.068 | −10.931 | −12.669 | 1.00 | 200.84 | E |
| ATOM | 14947 | CA | THR | E | 67 | 31.912 | −9.493 | −12.461 | 1.00 | 200.84 | E |
| ATOM | 14948 | CB | THR | E | 67 | 32.406 | −9.077 | −11.051 | 1.00 | 174.47 | E |
| ATOM | 14949 | OG1 | THR | E | 67 | 31.765 | −9.888 | −10.059 | 1.00 | 174.47 | E |
| ATOM | 14950 | CG2 | THR | E | 67 | 32.081 | −7.609 | −10.778 | 1.00 | 174.47 | E |
| ATOM | 14951 | C | THR | E | 67 | 32.628 | −8.626 | −13.494 | 1.00 | 200.84 | E |
| ATOM | 14952 | O | THR | E | 67 | 32.123 | −8.406 | −14.596 | 1.00 | 200.84 | E |
| ATOM | 14953 | N | HIS | E | 68 | 33.812 | −8.142 | −13.130 | 1.00 | 172.33 | E |
| ATOM | 14954 | CA | HIS | E | 68 | 34.583 | −7.281 | −14.011 | 1.00 | 167.45 | E |
| ATOM | 14955 | CB | HIS | E | 68 | 34.190 | −5.824 | −13.759 | 1.00 | 188.77 | E |
| ATOM | 14956 | CG | HIS | E | 68 | 34.490 | −4.907 | −14.901 | 1.00 | 188.77 | E |
| ATOM | 14957 | CD2 | HIS | E | 68 | 35.280 | −3.811 | −14.982 | 1.00 | 188.77 | E |
| ATOM | 14958 | ND1 | HIS | E | 68 | 33.923 | −5.064 | −16.148 | 1.00 | 188.77 | E |
| ATOM | 14959 | CE1 | HIS | E | 68 | 34.351 | −4.103 | −16.947 | 1.00 | 188.77 | E |
| ATOM | 14960 | NE2 | HIS | E | 68 | 35.174 | −3.329 | −16.264 | 1.00 | 188.77 | E |
| ATOM | 14961 | C | HIS | E | 68 | 36.077 | −7.468 | −13.763 | 1.00 | 163.28 | E |
| ATOM | 14962 | O | HIS | E | 68 | 36.507 | −7.662 | −12.627 | 1.00 | 162.73 | E |
| ATOM | 14963 | N | VAL | E | 69 | 36.862 | −7.412 | −14.835 | 1.00 | 109.20 | E |
| ATOM | 14964 | CA | VAL | E | 69 | 38.311 | −7.565 | −14.740 | 1.00 | 108.93 | E |
| ATOM | 14965 | CB | VAL | E | 69 | 38.807 | −8.798 | −15.540 | 1.00 | 130.79 | E |
| ATOM | 14966 | CG1 | VAL | E | 69 | 40.309 | −8.954 | −15.380 | 1.00 | 130.55 | E |
| ATOM | 14967 | CG2 | VAL | E | 69 | 38.097 | −10.051 | −15.064 | 1.00 | 129.05 | E |
| ATOM | 14968 | C | VAL | E | 69 | 39.004 | −6.319 | −15.287 | 1.00 | 108.52 | E |
| ATOM | 14969 | O | VAL | E | 69 | 38.644 | −5.807 | −16.350 | 1.00 | 112.08 | E |
| ATOM | 14970 | N | LEU | E | 70 | 39.993 | −5.828 | −14.547 | 1.00 | 83.16 | E |
| ATOM | 14971 | CA | LEU | E | 70 | 40.742 | −4.655 | −14.971 | 1.00 | 78.38 | E |
| ATOM | 14972 | CB | LEU | E | 70 | 40.736 | −3.576 | −13.889 | 1.00 | 73.01 | E |
| ATOM | 14973 | CG | LEU | E | 70 | 39.411 | −2.875 | −13.595 | 1.00 | 74.71 | E |
| ATOM | 14974 | CD1 | LEU | E | 70 | 39.653 | −1.779 | −12.575 | 1.00 | 69.75 | E |
| ATOM | 14975 | CD2 | LEU | E | 70 | 38.830 | −2.289 | −14.866 | 1.00 | 75.31 | E |
| ATOM | 14976 | C | LEU | E | 70 | 42.180 | −5.018 | −15.290 | 1.00 | 79.42 | E |
| ATOM | 14977 | O | LEU | E | 70 | 42.831 | −5.749 | −14.541 | 1.00 | 79.53 | E |
| ATOM | 14978 | N | LEU | E | 71 | 42.665 | −4.502 | −16.414 | 1.00 | 71.80 | E |
| ATOM | 14979 | CA | LEU | E | 71 | 44.033 | −4.740 | −16.849 | 1.00 | 71.74 | E |
| ATOM | 14980 | CB | LEU | E | 71 | 44.047 | −5.239 | −18.294 | 1.00 | 59.46 | E |
| ATOM | 14981 | CG | LEU | E | 71 | 43.297 | −6.545 | −18.562 | 1.00 | 57.13 | E |
| ATOM | 14982 | CD1 | LEU | E | 71 | 43.313 | −6.863 | −20.042 | 1.00 | 53.28 | E |
| ATOM | 14983 | CD2 | LEU | E | 71 | 43.941 | −7.668 | −17.776 | 1.00 | 57.25 | E |
| ATOM | 14984 | C | LEU | E | 71 | 44.797 | −3.426 | −16.753 | 1.00 | 75.77 | E |
| ATOM | 14985 | O | LEU | E | 71 | 44.360 | −2.411 | −17.294 | 1.00 | 75.26 | E |
| ATOM | 14986 | N | THR | E | 72 | 45.924 | −3.439 | −16.048 | 1.00 | 77.94 | E |
| ATOM | 14987 | CA | THR | E | 72 | 46.727 | −2.233 | −15.905 | 1.00 | 75.37 | E |
| ATOM | 14988 | CB | THR | E | 72 | 46.730 | −1.686 | −14.456 | 1.00 | 73.84 | E |
| ATOM | 14989 | OG1 | THR | E | 72 | 47.595 | −2.487 | −13.641 | 1.00 | 76.37 | E |
| ATOM | 14990 | CG2 | THR | E | 72 | 45.331 | −1.699 | −13.876 | 1.00 | 71.56 | E |
| ATOM | 14991 | C | THR | E | 72 | 48.168 | −2.487 | −16.297 | 1.00 | 78.00 | E |
| ATOM | 14992 | O | THR | E | 72 | 48.739 | −3.547 | −16.020 | 1.00 | 79.04 | E |
| ATOM | 14993 | N | HIS | E | 73 | 48.747 | −1.493 | −16.951 | 1.00 | 49.48 | E |
| ATOM | 14994 | CA | HIS | E | 73 | 50.128 | −1.553 | −17.388 | 1.00 | 49.93 | E |
| ATOM | 14995 | CB | HIS | E | 73 | 50.206 | −1.623 | −18.909 | 1.00 | 54.45 | E |
| ATOM | 14996 | CG | HIS | E | 73 | 51.597 | −1.779 | −19.425 | 1.00 | 57.32 | E |
| ATOM | 14997 | CD2 | HIS | E | 73 | 52.729 | −2.210 | −18.820 | 1.00 | 59.08 | E |
| ATOM | 14998 | ND1 | HIS | E | 73 | 51.939 | −1.499 | −20.730 | 1.00 | 61.93 | E |
| ATOM | 14999 | CE1 | HIS | E | 73 | 53.225 | −1.753 | −20.906 | 1.00 | 60.47 | E |
| ATOM | 15000 | NE2 | HIS | E | 73 | 53.727 | −2.185 | −19.763 | 1.00 | 60.64 | E |
| ATOM | 15001 | C | HIS | E | 73 | 50.716 | −0.248 | −16.910 | 1.00 | 49.01 | E |
| ATOM | 15002 | O | HIS | E | 73 | 50.115 | 0.804 | −17.106 | 1.00 | 44.87 | E |
| ATOM | 15003 | N | THR | E | 74 | 51.880 | −0.296 | −16.278 | 1.00 | 55.00 | E |
| ATOM | 15004 | CA | THR | E | 74 | 52.463 | 0.937 | −15.780 | 1.00 | 61.59 | E |
| ATOM | 15005 | CB | THR | E | 74 | 51.776 | 1.341 | −14.418 | 1.00 | 54.50 | E |
| ATOM | 15006 | OG1 | THR | E | 74 | 52.765 | 1.707 | −13.448 | 1.00 | 57.01 | E |
| ATOM | 15007 | CG2 | THR | E | 74 | 50.942 | 0.191 | −13.870 | 1.00 | 59.33 | E |
| ATOM | 15008 | C | THR | E | 74 | 53.985 | 0.890 | −15.638 | 1.00 | 60.44 | E |
| ATOM | 15009 | O | THR | E | 74 | 54.551 | −0.120 | −15.220 | 1.00 | 56.02 | E |
| ATOM | 15010 | N | ILE | E | 75 | 54.642 | 1.983 | −16.022 | 1.00 | 52.14 | E |
| ATOM | 15011 | CA | ILE | E | 75 | 56.087 | 2.081 | −15.910 | 1.00 | 53.44 | E |
| ATOM | 15012 | CB | ILE | E | 75 | 56.710 | 2.670 | −17.183 | 1.00 | 40.95 | E |
| ATOM | 15013 | CG2 | ILE | E | 75 | 58.203 | 2.878 | −16.995 | 1.00 | 40.44 | E |
| ATOM | 15014 | CG1 | ILE | E | 75 | 56.462 | 1.730 | −18.356 | 1.00 | 38.11 | E |
| ATOM | 15015 | CD1 | ILE | E | 75 | 57.057 | 2.218 | −19.651 | 1.00 | 41.40 | E |
| ATOM | 15016 | C | ILE | E | 75 | 56.389 | 2.997 | −14.736 | 1.00 | 56.58 | E |
| ATOM | 15017 | O | ILE | E | 75 | 55.940 | 4.152 | −14.709 | 1.00 | 53.35 | E |
| ATOM | 15018 | N | SER | E | 76 | 57.140 | 2.478 | −13.766 | 1.00 | 60.08 | E |
| ATOM | 15019 | CA | SER | E | 76 | 57.492 | 3.245 | −12.576 | 1.00 | 61.16 | E |
| ATOM | 15020 | CB | SER | E | 76 | 57.184 | 2.448 | −11.314 | 1.00 | 55.71 | E |
| ATOM | 15021 | OG | SER | E | 76 | 55.829 | 2.058 | −11.264 | 1.00 | 65.00 | E |
| ATOM | 15022 | C | SER | E | 76 | 58.956 | 3.619 | −12.529 | 1.00 | 57.16 | E |
| ATOM | 15023 | O | SER | E | 76 | 59.800 | 2.958 | −13.129 | 1.00 | 53.31 | E |
| ATOM | 15024 | N | ARG | E | 77 | 59.249 | 4.693 | −11.808 | 1.00 | 42.78 | E |
| ATOM | 15025 | CA | ARG | E | 77 | 60.618 | 5.130 | −11.627 | 1.00 | 47.96 | E |
| ATOM | 15026 | CB | ARG | E | 77 | 60.841 | 6.552 | −12.152 | 1.00 | 50.24 | E |
| ATOM | 15027 | CG | ARG | E | 77 | 62.053 | 7.215 | −11.501 | 1.00 | 52.42 | E |
| ATOM | 15028 | CD | ARG | E | 77 | 62.654 | 8.322 | −12.336 | 1.00 | 53.29 | E |
| ATOM | 15029 | NE | ARG | E | 77 | 62.397 | 9.661 | −11.812 | 1.00 | 55.73 | E |
| ATOM | 15030 | CZ | ARG | E | 77 | 63.352 | 10.552 | −11.553 | 1.00 | 59.16 | E |
| ATOM | 15031 | NH1 | ARG | E | 77 | 63.034 | 11.753 | −11.092 | 1.00 | 59.25 | E |
| ATOM | 15032 | NH2 | ARG | E | 77 | 64.629 | 10.238 | −11.739 | 1.00 | 59.02 | E |
| ATOM | 15033 | C | ARG | E | 77 | 60.873 | 5.118 | −10.137 | 1.00 | 46.30 | E |
| ATOM | 15034 | O | ARG | E | 77 | 60.040 | 5.574 | −9.354 | 1.00 | 52.05 | E |
| ATOM | 15035 | N | ILE | E | 78 | 61.999 | 4.549 | −9.736 | 1.00 | 44.77 | E |
| ATOM | 15036 | CA | ILE | E | 78 | 62.369 | 4.573 | −8.331 | 1.00 | 43.46 | E |
| ATOM | 15037 | CB | ILE | E | 78 | 62.450 | 3.125 | −7.685 | 1.00 | 72.29 | E |
| ATOM | 15038 | CG2 | ILE | E | 78 | 61.795 | 2.095 | −8.579 | 1.00 | 72.79 | E |
| ATOM | 15039 | CG1 | ILE | E | 78 | 63.886 | 2.739 | −7.374 | 1.00 | 73.96 | E |
| ATOM | 15040 | CD1 | ILE | E | 78 | 64.461 | 3.489 | −6.200 | 1.00 | 74.28 | E |
| ATOM | 15041 | C | ILE | E | 78 | 63.708 | 5.303 | −8.432 | 1.00 | 45.63 | E |
| ATOM | 15042 | O | ILE | E | 78 | 64.721 | 4.739 | −8.838 | 1.00 | 44.18 | E |
| ATOM | 15043 | N | ALA | E | 79 | 63.667 | 6.596 | −8.118 | 1.00 | 53.97 | E |
| ATOM | 15044 | CA | ALA | E | 79 | 64.837 | 7.457 | −8.216 | 1.00 | 50.57 | E |
| ATOM | 15045 | CB | ALA | E | 79 | 64.394 | 8.912 | −8.195 | 1.00 | 52.11 | E |
| ATOM | 15046 | C | ALA | E | 79 | 65.898 | 7.222 | −7.152 | 1.00 | 51.77 | E |
| ATOM | 15047 | O | ALA | E | 79 | 65.595 | 6.815 | −6.040 | 1.00 | 51.97 | E |
| ATOM | 15048 | N | VAL | E | 80 | 67.152 | 7.482 | −7.502 | 1.00 | 64.97 | E |
| ATOM | 15049 | CA | VAL | E | 80 | 68.250 | 7.311 | −6.560 | 1.00 | 67.46 | E |
| ATOM | 15050 | CB | VAL | E | 80 | 69.610 | 7.599 | −7.223 | 1.00 | 75.48 | E |
| ATOM | 15051 | CG1 | VAL | E | 80 | 70.724 | 7.459 | −6.202 | 1.00 | 73.88 | E |
| ATOM | 15052 | CG2 | VAL | E | 80 | 69.836 | 6.649 | −8.374 | 1.00 | 77.18 | E |
| ATOM | 15053 | C | VAL | E | 80 | 68.072 | 8.278 | −5.398 | 1.00 | 72.01 | E |
| ATOM | 15054 | O | VAL | E | 80 | 68.426 | 7.969 | −4.262 | 1.00 | 70.60 | E |
| ATOM | 15055 | N | SER | E | 81 | 67.515 | 9.451 | −5.685 | 1.00 | 68.09 | E |
| ATOM | 15056 | CA | SER | E | 81 | 67.305 | 10.461 | −4.656 | 1.00 | 70.95 | E |
| ATOM | 15057 | CB | SER | E | 81 | 67.505 | 11.858 | −5.239 | 1.00 | 52.47 | E |
| ATOM | 15058 | OG | SER | E | 81 | 68.851 | 12.059 | −5.626 | 1.00 | 55.51 | E |
| ATOM | 15059 | C | SER | E | 81 | 65.941 | 10.389 | −3.973 | 1.00 | 75.13 | E |
| ATOM | 15060 | O | SER | E | 81 | 65.598 | 11.278 | −3.193 | 1.00 | 75.65 | E |
| ATOM | 15061 | N | TYR | E | 82 | 65.169 | 9.340 | −4.261 | 1.00 | 52.08 | E |
| ATOM | 15062 | CA | TYR | E | 82 | 63.848 | 9.158 | −3.654 | 1.00 | 54.12 | E |
| ATOM | 15063 | CB | TYR | E | 82 | 62.849 | 10.166 | −4.230 | 1.00 | 55.25 | E |
| ATOM | 15064 | CG | TYR | E | 82 | 61.528 | 10.215 | −3.489 | 1.00 | 58.45 | E |
| ATOM | 15065 | CD1 | TYR | E | 82 | 60.330 | 9.973 | −4.147 | 1.00 | 60.74 | E |
| ATOM | 15066 | CE1 | TYR | E | 82 | 59.122 | 9.987 | −3.465 | 1.00 | 62.53 | E |
| ATOM | 15067 | CD2 | TYR | E | 82 | 61.482 | 10.479 | −2.123 | 1.00 | 60.54 | E |
| ATOM | 15068 | CE2 | TYR | E | 82 | 60.281 | 10.498 | −1.433 | 1.00 | 62.01 | E |
| ATOM | 15069 | CZ | TYR | E | 82 | 59.104 | 10.247 | −2.108 | 1.00 | 62.70 | E |
| ATOM | 15070 | OH | TYR | E | 82 | 57.909 | 10.235 | −1.424 | 1.00 | 61.12 | E |
| ATOM | 15071 | C | TYR | E | 82 | 63.343 | 7.742 | −3.903 | 1.00 | 53.55 | E |
| ATOM | 15072 | O | TYR | E | 82 | 62.443 | 7.535 | −4.715 | 1.00 | 53.73 | E |
| ATOM | 15073 | N | GLN | E | 83 | 63.930 | 6.779 | −3.190 | 1.00 | 61.00 | E |
| ATOM | 15074 | CA | GLN | E | 83 | 63.601 | 5.361 | −3.317 | 1.00 | 60.89 | E |
| ATOM | 15075 | CB | GLN | E | 83 | 64.401 | 4.532 | −2.315 | 1.00 | 77.95 | E |
| ATOM | 15076 | CG | GLN | E | 83 | 65.649 | 3.877 | −2.852 | 1.00 | 82.22 | E |
| ATOM | 15077 | CD | GLN | E | 83 | 66.794 | 4.847 | −3.011 | 1.00 | 83.26 | E |
| ATOM | 15078 | OE1 | GLN | E | 83 | 67.938 | 4.438 | −3.203 | 1.00 | 82.28 | E |
| ATOM | 15079 | NE2 | GLN | E | 83 | 66.497 | 6.143 | −2.935 | 1.00 | 80.76 | E |
| ATOM | 15080 | C | GLN | E | 83 | 62.138 | 4.975 | −3.164 | 1.00 | 59.07 | E |
| ATOM | 15081 | O | GLN | E | 83 | 61.765 | 4.375 | −2.168 | 1.00 | 56.52 | E |
| ATOM | 15082 | N | THR | E | 84 | 61.304 | 5.289 | −4.144 | 1.00 | 84.05 | E |
| ATOM | 15083 | CA | THR | E | 84 | 59.902 | 4.910 | −4.064 | 1.00 | 76.42 | E |
| ATOM | 15084 | CB | THR | E | 84 | 59.069 | 5.915 | −3.276 | 1.00 | 91.69 | E |
| ATOM | 15085 | OG1 | THR | E | 84 | 59.698 | 6.170 | −2.020 | 1.00 | 92.24 | E |
| ATOM | 15086 | CG2 | THR | E | 84 | 57.673 | 5.364 | −3.035 | 1.00 | 94.44 | E |
| ATOM | 15087 | C | THR | E | 84 | 59.336 | 4.872 | −5.456 | 1.00 | 72.93 | E |
| ATOM | 15088 | O | THR | E | 84 | 59.611 | 5.750 | −6.271 | 1.00 | 74.24 | E |
| ATOM | 15089 | N | LYS | E | 85 | 58.545 | 3.850 | −5.735 | 1.00 | 53.81 | E |
| ATOM | 15090 | CA | LYS | E | 85 | 57.930 | 3.743 | −7.037 | 1.00 | 53.67 | E |
| ATOM | 15091 | CB | LYS | E | 85 | 57.099 | 2.466 | −7.140 | 1.00 | 82.64 | E |
| ATOM | 15092 | CG | LYS | E | 85 | 57.817 | 1.259 | −7.703 | 1.00 | 79.58 | E |
| ATOM | 15093 | CD | LYS | E | 85 | 56.807 | 0.143 | −7.952 | 1.00 | 77.98 | E |
| ATOM | 15094 | CE | LYS | E | 85 | 57.436 | −1.038 | −8.668 | 1.00 | 76.28 | E |
| ATOM | 15095 | NZ | LYS | E | 85 | 58.525 | −1.683 | −7.877 | 1.00 | 74.53 | E |
| ATOM | 15096 | C | LYS | E | 85 | 56.999 | 4.928 | −7.254 | 1.00 | 53.47 | E |
| ATOM | 15097 | O | LYS | E | 85 | 56.105 | 5.166 | −6.448 | 1.00 | 53.92 | E |
| ATOM | 15098 | N | VAL | E | 86 | 57.235 | 5.684 | −8.325 | 1.00 | 57.16 | E |
| ATOM | 15099 | CA | VAL | E | 86 | 56.360 | 6.792 | −8.699 | 1.00 | 60.05 | E |
| ATOM | 15100 | CB | VAL | E | 86 | 57.034 | 8.199 | −8.550 | 1.00 | 65.03 | E |
| ATOM | 15101 | CG1 | VAL | E | 86 | 57.383 | 8.451 | −7.100 | 1.00 | 71.77 | E |
| ATOM | 15102 | CG2 | VAL | E | 86 | 58.267 | 8.300 | −9.399 | 1.00 | 66.30 | E |
| ATOM | 15103 | C | VAL | E | 86 | 56.031 | 6.495 | −10.157 | 1.00 | 60.72 | E |
| ATOM | 15104 | O | VAL | E | 86 | 56.918 | 6.238 | −10.963 | 1.00 | 65.41 | E |
| ATOM | 15105 | N | ASN | E | 87 | 54.750 | 6.486 | −10.483 | 1.00 | 61.40 | E |
| ATOM | 15106 | CA | ASN | E | 87 | 54.333 | 6.188 | −11.842 | 1.00 | 58.08 | E |
| ATOM | 15107 | CB | ASN | E | 87 | 52.828 | 5.937 | −11.864 | 1.00 | 56.96 | E |
| ATOM | 15108 | CG | ASN | E | 87 | 52.444 | 4.669 | −11.142 | 1.00 | 57.26 | E |
| ATOM | 15109 | OD1 | ASN | E | 87 | 53.302 | 3.886 | −10.732 | 1.00 | 59.87 | E |
| ATOM | 15110 | ND2 | ASN | E | 87 | 51.148 | 4.448 | −10.995 | 1.00 | 54.22 | E |
| ATOM | 15111 | C | ASN | E | 87 | 54.684 | 7.259 | −12.868 | 1.00 | 56.90 | E |
| ATOM | 15112 | O | ASN | E | 87 | 54.418 | 8.443 | −12.651 | 1.00 | 55.48 | E |
| ATOM | 15113 | N | LEU | E | 88 | 55.274 | 6.845 | −13.989 | 1.00 | 57.59 | E |
| ATOM | 15114 | CA | LEU | E | 88 | 55.618 | 7.785 | −15.054 | 1.00 | 56.58 | E |
| ATOM | 15115 | CB | LEU | E | 88 | 56.984 | 7.477 | −15.650 | 1.00 | 35.78 | E |
| ATOM | 15116 | CG | LEU | E | 88 | 58.179 | 7.553 | −14.720 | 1.00 | 32.71 | E |
| ATOM | 15117 | CD1 | LEU | E | 88 | 59.432 | 7.248 | −15.518 | 1.00 | 33.48 | E |
| ATOM | 15118 | CD2 | LEU | E | 88 | 58.242 | 8.927 | −14.084 | 1.00 | 31.28 | E |
| ATOM | 15119 | C | LEU | E | 88 | 54.586 | 7.670 | −16.157 | 1.00 | 55.82 | E |
| ATOM | 15120 | O | LEU | E | 88 | 54.119 | 8.667 | −16.695 | 1.00 | 54.29 | E |
| ATOM | 15121 | N | LEU | E | 89 | 54.234 | 6.435 | −16.487 | 1.00 | 51.56 | E |
| ATOM | 15122 | CA | LEU | E | 89 | 53.264 | 6.167 | −17.532 | 1.00 | 50.48 | E |
| ATOM | 15123 | CB | LEU | E | 89 | 53.987 | 5.737 | −18.808 | 1.00 | 42.69 | E |
| ATOM | 15124 | CG | LEU | E | 89 | 55.088 | 6.659 | −19.325 | 1.00 | 46.67 | E |
| ATOM | 15125 | CD1 | LEU | E | 89 | 55.995 | 5.860 | −20.242 | 1.00 | 46.40 | E |
| ATOM | 15126 | CD2 | LEU | E | 89 | 54.496 | 7.847 | −20.046 | 1.00 | 45.79 | E |
| ATOM | 15127 | C | LEU | E | 89 | 52.364 | 5.040 | −17.046 | 1.00 | 52.15 | E |
| ATOM | 15128 | O | LEU | E | 89 | 52.837 | 4.091 | −16.418 | 1.00 | 52.87 | E |
| ATOM | 15129 | N | SER | E | 90 | 51.074 | 5.137 | −17.340 | 1.00 | 44.09 | E |
| ATOM | 15130 | CA | SER | E | 90 | 50.139 | 4.114 | −16.913 | 1.00 | 43.43 | E |
| ATOM | 15131 | CB | SER | E | 90 | 49.812 | 4.301 | −15.436 | 1.00 | 75.67 | E |
| ATOM | 15132 | OG | SER | E | 90 | 48.839 | 3.361 | −15.015 | 1.00 | 79.89 | E |
| ATOM | 15133 | C | SER | E | 90 | 48.849 | 4.138 | −17.705 | 1.00 | 42.06 | E |
| ATOM | 15134 | O | SER | E | 90 | 48.386 | 5.194 | −18.117 | 1.00 | 41.88 | E |
| ATOM | 15135 | N | ALA | E | 91 | 48.261 | 2.970 | −17.918 | 1.00 | 49.67 | E |
| ATOM | 15136 | CA | ALA | E | 91 | 47.000 | 2.896 | −18.636 | 1.00 | 47.76 | E |
| ATOM | 15137 | CB | ALA | E | 91 | 47.248 | 2.693 | −20.115 | 1.00 | 34.73 | E |
| ATOM | 15138 | C | ALA | E | 91 | 46.177 | 1.751 | −18.066 | 1.00 | 51.73 | E |
| ATOM | 15139 | O | ALA | E | 91 | 46.721 | 0.834 | −17.435 | 1.00 | 53.60 | E |
| ATOM | 15140 | N | ILE | E | 92 | 44.865 | 1.818 | −18.275 | 1.00 | 60.53 | E |
| ATOM | 15141 | CA | ILE | E | 92 | 43.948 | 0.794 | −17.783 | 1.00 | 60.51 | E |
| ATOM | 15142 | CB | ILE | E | 92 | 43.077 | 1.342 | −16.651 | 1.00 | 69.86 | E |
| ATOM | 15143 | CG2 | ILE | E | 92 | 42.236 | 0.233 | −16.072 | 1.00 | 75.93 | E |
| ATOM | 15144 | CG1 | ILE | E | 92 | 43.961 | 1.940 | −15.557 | 1.00 | 67.97 | E |
| ATOM | 15145 | CD1 | ILE | E | 92 | 43.196 | 2.666 | −14.477 | 1.00 | 73.84 | E |
| ATOM | 15146 | C | ILE | E | 92 | 43.040 | 0.360 | −18.917 | 1.00 | 60.34 | E |
| ATOM | 15147 | O | ILE | E | 92 | 42.701 | 1.171 | −19.775 | 1.00 | 61.42 | E |
| ATOM | 15148 | N | LYS | E | 93 | 42.641 | −0.907 | −18.929 | 1.00 | 76.72 | E |
| ATOM | 15149 | CA | LYS | E | 93 | 41.771 | −1.398 | −19.987 | 1.00 | 77.37 | E |
| ATOM | 15150 | CB | LYS | E | 93 | 42.615 | −2.002 | −21.113 | 1.00 | 87.35 | E |
| ATOM | 15151 | CG | LYS | E | 93 | 41.988 | −1.884 | −22.491 | 1.00 | 91.04 | E |
| ATOM | 15152 | CD | LYS | E | 93 | 43.050 | −1.935 | −23.583 | 1.00 | 90.81 | E |
| ATOM | 15153 | CE | LYS | E | 93 | 42.455 | −1.671 | −24.974 | 1.00 | 90.71 | E |
| ATOM | 15154 | NZ | LYS | E | 93 | 43.480 | −1.654 | −26.072 | 1.00 | 93.28 | E |
| ATOM | 15155 | C | LYS | E | 93 | 40.816 | −2.435 | −19.418 | 1.00 | 79.07 | E |
| ATOM | 15156 | O | LYS | E | 93 | 41.225 | −3.304 | −18.644 | 1.00 | 76.99 | E |
| ATOM | 15157 | N | SER | E | 94 | 39.544 | −2.331 | −19.796 | 1.00 | 74.63 | E |
| ATOM | 15158 | CA | SER | E | 94 | 38.502 | −3.249 | −19.329 | 1.00 | 77.08 | E |
| ATOM | 15159 | CB | SER | E | 94 | 37.284 | −2.453 | −18.856 | 1.00 | 119.38 | E |
| ATOM | 15160 | OG | SER | E | 94 | 36.770 | −1.634 | −19.897 | 1.00 | 121.81 | E |
| ATOM | 15161 | C | SER | E | 94 | 38.076 | −4.206 | −20.439 | 1.00 | 79.67 | E |
| ATOM | 15162 | O | SER | E | 94 | 37.218 | −3.873 | −21.258 | 1.00 | 80.94 | E |
| ATOM | 15163 | N | PRO | E | 95 | 38.653 | −5.419 | −20.465 | 1.00 | 68.27 | E |
| ATOM | 15164 | CD | PRO | E | 95 | 39.494 | −6.002 | −19.404 | 1.00 | 117.08 | E |
| ATOM | 15165 | CA | PRO | E | 95 | 38.333 | −6.425 | −21.484 | 1.00 | 72.21 | E |
| ATOM | 15166 | CB | PRO | E | 95 | 39.306 | −7.553 | −21.160 | 1.00 | 120.02 | E |
| ATOM | 15167 | CG | PRO | E | 95 | 39.377 | −7.490 | −19.673 | 1.00 | 117.47 | E |
| ATOM | 15168 | C | PRO | E | 95 | 36.879 | −6.875 | −21.435 | 1.00 | 76.64 | E |
| ATOM | 15169 | O | PRO | E | 95 | 36.054 | −6.456 | −22.251 | 1.00 | 80.07 | E |
| ATOM | 15170 | N | CYS | E | 96 | 36.573 | −7.737 | −20.474 | 1.00 | 183.54 | E |
| ATOM | 15171 | CA | CYS | E | 96 | 35.225 | −8.245 | −20.316 | 1.00 | 183.33 | E |
| ATOM | 15172 | C | CYS | E | 96 | 34.355 | −7.220 | −19.618 | 1.00 | 184.43 | E |
| ATOM | 15173 | O | CYS | E | 96 | 34.497 | −6.974 | −18.421 | 1.00 | 184.73 | E |
| ATOM | 15174 | CB | CYS | E | 96 | 35.246 | −9.548 | −19.522 | 1.00 | 200.84 | E |
| ATOM | 15175 | SG | CYS | E | 96 | 35.997 | −10.928 | −20.440 | 1.00 | 200.84 | E |
| ATOM | 15176 | N | GLN | E | 97 | 33.460 | −6.614 | −20.387 | 1.00 | 200.40 | E |
| ATOM | 15177 | CA | GLN | E | 97 | 32.544 | −5.613 | −19.866 | 1.00 | 200.40 | E |
| ATOM | 15178 | CB | GLN | E | 97 | 31.970 | −4.794 | −21.024 | 1.00 | 143.65 | E |
| ATOM | 15179 | CG | GLN | E | 97 | 33.031 | −4.213 | −21.946 | 1.00 | 143.65 | E |
| ATOM | 15180 | CD | GLN | E | 97 | 32.466 | −3.776 | −23.286 | 1.00 | 143.65 | E |
| ATOM | 15181 | OE1 | GLN | E | 97 | 31.572 | −2.932 | −23.353 | 1.00 | 143.65 | E |
| ATOM | 15182 | NE2 | GLN | E | 97 | 32.988 | −4.351 | −24.364 | 1.00 | 143.65 | E |
| ATOM | 15183 | C | GLN | E | 97 | 31.419 | −6.328 | −19.120 | 1.00 | 200.40 | E |
| ATOM | 15184 | O | GLN | E | 97 | 30.247 | −5.989 | −19.282 | 1.00 | 200.40 | E |
| ATOM | 15185 | N | ARG | E | 98 | 31.791 | −7.324 | −18.314 | 1.00 | 200.84 | E |
| ATOM | 15186 | CA | ARG | E | 98 | 30.841 | −8.117 | −17.529 | 1.00 | 200.84 | E |
| ATOM | 15187 | CB | ARG | E | 98 | 29.893 | −7.184 | −16.756 | 1.00 | 189.26 | E |
| ATOM | 15188 | CG | ARG | E | 98 | 28.744 | −7.876 | −16.030 | 1.00 | 189.26 | E |
| ATOM | 15189 | CD | ARG | E | 98 | 29.227 | −8.810 | −14.934 | 1.00 | 189.26 | E |
| ATOM | 15190 | NE | ARG | E | 98 | 28.135 | −9.622 | −14.403 | 1.00 | 189.26 | E |
| ATOM | 15191 | CZ | ARG | E | 98 | 27.013 | −9.129 | −13.888 | 1.00 | 189.26 | E |
| ATOM | 15192 | NH1 | ARG | E | 98 | 26.078 | −9.951 | −13.431 | 1.00 | 189.26 | E |
| ATOM | 15193 | NH2 | ARG | E | 98 | 26.823 | −7.817 | −13.827 | 1.00 | 189.26 | E |
| ATOM | 15194 | C | ARG | E | 98 | 30.041 | −9.102 | −18.397 | 1.00 | 200.84 | E |
| ATOM | 15195 | O | ARG | E | 98 | 29.177 | −9.825 | −17.896 | 1.00 | 200.84 | E |
| ATOM | 15196 | N | GLU | E | 99 | 30.347 | −9.138 | −19.695 | 1.00 | 200.84 | E |
| ATOM | 15197 | CA | GLU | E | 99 | 29.662 | −10.032 | −20.630 | 1.00 | 200.84 | E |
| ATOM | 15198 | CB | GLU | E | 99 | 30.458 | −10.167 | −21.935 | 1.00 | 164.54 | E |
| ATOM | 15199 | CG | GLU | E | 99 | 31.834 | −9.514 | −21.916 | 1.00 | 164.54 | E |
| ATOM | 15200 | CD | GLU | E | 99 | 31.788 | −8.038 | −22.267 | 1.00 | 164.54 | E |
| ATOM | 15201 | OE1 | GLU | E | 99 | 30.894 | −7.332 | −21.754 | 1.00 | 164.54 | E |
| ATOM | 15202 | OE2 | GLU | E | 99 | 32.651 | −7.582 | −23.049 | 1.00 | 164.54 | E |
| ATOM | 15203 | C | GLU | E | 99 | 29.434 | −11.422 | −20.041 | 1.00 | 200.84 | E |
| ATOM | 15204 | O | GLU | E | 99 | 28.317 | −11.936 | −20.076 | 1.00 | 200.84 | E |
| ATOM | 15205 | N | THR | E | 100 | 30.498 | −12.016 | −19.502 | 1.00 | 200.84 | E |
| ATOM | 15206 | CA | THR | E | 100 | 30.448 | −13.349 | −18.900 | 1.00 | 200.84 | E |
| ATOM | 15207 | CB | THR | E | 100 | 29.760 | −13.319 | −17.508 | 1.00 | 168.16 | E |
| ATOM | 15208 | OG1 | THR | E | 100 | 28.455 | −12.742 | −17.625 | 1.00 | 168.44 | E |
| ATOM | 15209 | CG2 | THR | E | 100 | 30.581 | −12.502 | −16.520 | 1.00 | 169.33 | E |
| ATOM | 15210 | C | THR | E | 100 | 29.730 | −14.367 | −19.789 | 1.00 | 200.84 | E |
| ATOM | 15211 | O | THR | E | 100 | 28.534 | −14.251 | −20.050 | 1.00 | 200.84 | E |
| ATOM | 15212 | N | PRO | E | 101 | 30.462 | −15.389 | −20.261 | 1.00 | 190.62 | E |
| ATOM | 15213 | CD | PRO | E | 101 | 31.871 | −15.675 | −19.939 | 1.00 | 132.11 | E |
| ATOM | 15214 | CA | PRO | E | 101 | 29.909 | −16.436 | −21.124 | 1.00 | 190.62 | E |
| ATOM | 15215 | CB | PRO | E | 101 | 30.971 | −17.528 | −21.052 | 1.00 | 133.06 | E |
| ATOM | 15216 | CG | PRO | E | 101 | 32.230 | −16.735 | −20.961 | 1.00 | 131.09 | E |
| ATOM | 15217 | C | PRO | E | 101 | 28.532 | −16.928 | −20.683 | 1.00 | 190.62 | E |
| ATOM | 15218 | O | PRO | E | 101 | 28.393 | −17.573 | −19.644 | 1.00 | 190.62 | E |
| ATOM | 15219 | N | GLU | E | 102 | 27.522 | −16.612 | −21.487 | 1.00 | 180.84 | E |
| ATOM | 15220 | CA | GLU | E | 102 | 26.145 | −17.008 | −21.216 | 1.00 | 180.84 | E |
| ATOM | 15221 | CB | GLU | E | 102 | 25.289 | −16.749 | −22.465 | 1.00 | 150.09 | E |
| ATOM | 15222 | CG | GLU | E | 102 | 23.834 | −17.189 | −22.366 | 1.00 | 151.29 | E |
| ATOM | 15223 | CD | GLU | E | 102 | 23.666 | −18.697 | −22.446 | 1.00 | 152.60 | E |
| ATOM | 15224 | OE1 | GLU | E | 102 | 24.074 | −19.292 | −23.468 | 1.00 | 153.41 | E |
| ATOM | 15225 | OE2 | GLU | E | 102 | 23.125 | −19.285 | −21.485 | 1.00 | 151.72 | E |
| ATOM | 15226 | C | GLU | E | 102 | 26.043 | −18.476 | −20.790 | 1.00 | 177.40 | E |
| ATOM | 15227 | O | GLU | E | 102 | 25.267 | −18.820 | −19.897 | 1.00 | 174.46 | E |
| ATOM | 15228 | N | GLY | E | 103 | 26.835 | −19.335 | −21.427 | 1.00 | 125.36 | E |
| ATOM | 15229 | CA | GLY | E | 103 | 26.815 | −20.753 | −21.106 | 1.00 | 122.68 | E |
| ATOM | 15230 | C | GLY | E | 103 | 27.144 | −21.607 | −22.318 | 1.00 | 120.55 | E |
| ATOM | 15231 | O | GLY | E | 103 | 27.381 | −22.812 | −22.202 | 1.00 | 115.04 | E |
| ATOM | 15232 | N | ALA | E | 104 | 27.155 | −20.975 | −23.488 | 1.00 | 185.08 | E |
| ATOM | 15233 | CA | ALA | E | 104 | 27.460 | −21.659 | −24.739 | 1.00 | 185.08 | E |
| ATOM | 15234 | CB | ALA | E | 104 | 26.166 | −21.953 | −25.505 | 1.00 | 87.10 | E |
| ATOM | 15235 | C | ALA | E | 104 | 28.394 | −20.798 | −25.590 | 1.00 | 185.08 | E |
| ATOM | 15236 | O | ALA | E | 104 | 28.015 | −19.708 | −26.018 | 1.00 | 185.08 | E |
| ATOM | 15237 | N | GLU | E | 105 | 29.610 | −21.300 | −25.814 | 1.00 | 174.09 | E |
| ATOM | 15238 | CA | GLU | E | 105 | 30.653 | −20.635 | −26.608 | 1.00 | 174.09 | E |
| ATOM | 15239 | CB | GLU | E | 105 | 30.044 | −19.649 | −27.615 | 1.00 | 155.26 | E |
| ATOM | 15240 | CG | GLU | E | 105 | 29.235 | −20.301 | −28.723 | 1.00 | 155.26 | E |
| ATOM | 15241 | CD | GLU | E | 105 | 30.065 | −21.251 | −29.563 | 1.00 | 155.26 | E |
| ATOM | 15242 | OE1 | GLU | E | 105 | 30.535 | −22.273 | −29.022 | 1.00 | 155.26 | E |
| ATOM | 15243 | OE2 | GLU | E | 105 | 30.251 | −20.973 | −30.765 | 1.00 | 154.03 | E |
| ATOM | 15244 | C | GLU | E | 105 | 31.713 | −19.914 | −25.774 | 1.00 | 174.09 | E |
| ATOM | 15245 | O | GLU | E | 105 | 32.398 | −20.533 | −24.957 | 1.00 | 174.09 | E |
| ATOM | 15246 | N | ALA | E | 106 | 31.846 | −18.608 | −26.006 | 1.00 | 166.37 | E |
| ATOM | 15247 | CA | ALA | E | 106 | 32.810 | −17.748 | −25.314 | 1.00 | 166.37 | E |
| ATOM | 15248 | CB | ALA | E | 106 | 32.981 | −18.191 | −23.854 | 1.00 | 81.77 | E |
| ATOM | 15249 | C | ALA | E | 106 | 34.165 | −17.727 | −26.021 | 1.00 | 166.37 | E |
| ATOM | 15250 | O | ALA | E | 106 | 35.092 | −18.437 | −25.629 | 1.00 | 166.37 | E |
| ATOM | 15251 | N | LYS | E | 107 | 34.270 | −16.905 | −27.064 | 1.00 | 158.78 | E |
| ATOM | 15252 | CA | LYS | E | 107 | 35.502 | −16.769 | −27.843 | 1.00 | 152.90 | E |
| ATOM | 15253 | CB | LYS | E | 107 | 35.244 | −15.939 | −29.109 | 1.00 | 185.17 | E |
| ATOM | 15254 | CG | LYS | E | 107 | 34.504 | −16.657 | −30.236 | 1.00 | 185.12 | E |
| ATOM | 15255 | CD | LYS | E | 107 | 33.082 | −17.031 | −29.857 | 1.00 | 186.55 | E |
| ATOM | 15256 | CE | LYS | E | 107 | 32.323 | −17.559 | −31.063 | 1.00 | 182.59 | E |
| ATOM | 15257 | NZ | LYS | E | 107 | 33.032 | −18.694 | −31.716 | 1.00 | 182.85 | E |
| ATOM | 15258 | C | LYS | E | 107 | 36.612 | −16.100 | −27.032 | 1.00 | 148.38 | E |
| ATOM | 15259 | O | LYS | E | 107 | 36.363 | −15.563 | −25.952 | 1.00 | 147.51 | E |
| ATOM | 15260 | N | PRO | E | 108 | 37.858 | −16.134 | −27.541 | 1.00 | 101.33 | E |
| ATOM | 15261 | CD | PRO | E | 108 | 38.334 | −16.909 | −28.704 | 1.00 | 90.61 | E |
| ATOM | 15262 | CA | PRO | E | 108 | 38.987 | −15.515 | −26.841 | 1.00 | 100.07 | E |
| ATOM | 15263 | CB | PRO | E | 108 | 40.189 | −16.268 | −27.402 | 1.00 | 89.76 | E |
| ATOM | 15264 | CG | PRO | E | 108 | 39.794 | −16.472 | −28.824 | 1.00 | 87.14 | E |
| ATOM | 15265 | C | PRO | E | 108 | 39.047 | −14.019 | −27.146 | 1.00 | 99.30 | E |
| ATOM | 15266 | O | PRO | E | 108 | 38.480 | −13.558 | −28.139 | 1.00 | 97.48 | E |
| ATOM | 15267 | N | TRP | E | 109 | 39.718 | −13.264 | −26.282 | 1.00 | 106.66 | E |
| ATOM | 15268 | CA | TRP | E | 109 | 39.856 | −11.827 | −26.479 | 1.00 | 104.11 | E |
| ATOM | 15269 | CB | TRP | E | 109 | 38.985 | −11.048 | −25.493 | 1.00 | 96.92 | E |
| ATOM | 15270 | CG | TRP | E | 109 | 39.218 | −11.385 | −24.050 | 1.00 | 96.61 | E |
| ATOM | 15271 | CD2 | TRP | E | 109 | 40.243 | −10.861 | −23.193 | 1.00 | 95.62 | E |
| ATOM | 15272 | CE2 | TRP | E | 109 | 40.068 | −11.452 | −21.922 | 1.00 | 94.87 | E |
| ATOM | 15273 | CE3 | TRP | E | 109 | 41.292 | −9.951 | −23.375 | 1.00 | 95.72 | E |
| ATOM | 15274 | CD1 | TRP | E | 109 | 38.490 | −12.248 | −23.286 | 1.00 | 93.71 | E |
| ATOM | 15275 | NE1 | TRP | E | 109 | 38.991 | −12.293 | −22.008 | 1.00 | 92.03 | E |
| ATOM | 15276 | CZ2 | TRP | E | 109 | 40.902 | −11.165 | −20.838 | 1.00 | 91.32 | E |
| ATOM | 15277 | CZ3 | TRP | E | 109 | 42.123 | −9.666 | −22.294 | 1.00 | 95.48 | E |
| ATOM | 15278 | CH2 | TRP | E | 109 | 41.920 | −10.272 | −21.043 | 1.00 | 91.12 | E |
| ATOM | 15279 | C | TRP | E | 109 | 41.299 | −11.373 | −26.331 | 1.00 | 105.72 | E |
| ATOM | 15280 | O | TRP | E | 109 | 42.061 | −11.917 | −25.525 | 1.00 | 107.33 | E |
| ATOM | 15281 | N | TYR | E | 110 | 41.665 | −10.375 | −27.128 | 1.00 | 64.64 | E |
| ATOM | 15282 | CA | TYR | E | 110 | 43.009 | −9.818 | −27.098 | 1.00 | 63.28 | E |
| ATOM | 15283 | CB | TYR | E | 110 | 43.710 | −10.037 | −28.435 | 1.00 | 69.92 | E |
| ATOM | 15284 | CG | TYR | E | 110 | 43.943 | −11.487 | −28.793 | 1.00 | 73.47 | E |
| ATOM | 15285 | CD1 | TYR | E | 110 | 42.889 | −12.309 | −29.202 | 1.00 | 77.56 | E |
| ATOM | 15286 | CE1 | TYR | E | 110 | 43.115 | −13.637 | −29.573 | 1.00 | 77.88 | E |
| ATOM | 15287 | CD2 | TYR | E | 110 | 45.230 | −12.032 | −28.757 | 1.00 | 73.70 | E |
| ATOM | 15288 | CE2 | TYR | E | 110 | 45.464 | −13.353 | −29.125 | 1.00 | 79.07 | E |
| ATOM | 15289 | CZ | TYR | E | 110 | 44.408 | −14.149 | −29.532 | 1.00 | 79.36 | E |
| ATOM | 15290 | OH | TYR | E | 110 | 44.665 | −15.449 | −29.905 | 1.00 | 80.56 | E |
| ATOM | 15291 | C | TYR | E | 110 | 42.916 | −8.325 | −26.811 | 1.00 | 61.79 | E |
| ATOM | 15292 | O | TYR | E | 110 | 42.051 | −7.633 | −27.349 | 1.00 | 62.98 | E |
| ATOM | 15293 | N | GLU | E | 111 | 43.813 | −7.836 | −25.964 | 1.00 | 57.15 | E |
| ATOM | 15294 | CA | GLU | E | 111 | 43.835 | −6.427 | −25.595 | 1.00 | 55.17 | E |
| ATOM | 15295 | CB | GLU | E | 111 | 43.028 | −6.219 | −24.317 | 1.00 | 77.91 | E |
| ATOM | 15296 | CG | GLU | E | 111 | 42.178 | −4.985 | −24.342 | 1.00 | 84.83 | E |
| ATOM | 15297 | CD | GLU | E | 111 | 41.158 | −5.021 | −25.456 | 1.00 | 89.80 | E |
| ATOM | 15298 | OE1 | GLU | E | 111 | 40.330 | −5.955 | −25.465 | 1.00 | 90.01 | E |
| ATOM | 15299 | OE2 | GLU | E | 111 | 41.180 | −4.119 | −26.322 | 1.00 | 96.96 | E |
| ATOM | 15300 | C | GLU | E | 111 | 45.270 | −5.951 | −25.371 | 1.00 | 55.75 | E |
| ATOM | 15301 | O | GLU | E | 111 | 45.922 | −6.338 | −24.396 | 1.00 | 61.11 | E |
| ATOM | 15302 | N | PRO | E | 112 | 45.793 | −5.124 | −26.289 | 1.00 | 57.56 | E |
| ATOM | 15303 | CD | PRO | E | 112 | 45.274 | −4.911 | −27.655 | 1.00 | 52.25 | E |
| ATOM | 15304 | CA | PRO | E | 112 | 47.161 | −4.602 | −26.169 | 1.00 | 56.79 | E |
| ATOM | 15305 | CB | PRO | E | 112 | 47.613 | −4.524 | −27.613 | 1.00 | 48.32 | E |
| ATOM | 15306 | CG | PRO | E | 112 | 46.357 | −4.036 | −28.287 | 1.00 | 49.45 | E |
| ATOM | 15307 | C | PRO | E | 112 | 47.187 | −3.227 | −25.509 | 1.00 | 53.82 | E |
| ATOM | 15308 | O | PRO | E | 112 | 46.196 | −2.507 | −25.535 | 1.00 | 53.95 | E |
| ATOM | 15309 | N | ILE | E | 113 | 48.321 | −2.877 | −24.914 | 1.00 | 59.33 | E |
| ATOM | 15310 | CA | ILE | E | 113 | 48.482 | −1.574 | −24.279 | 1.00 | 61.30 | E |
| ATOM | 15311 | CB | ILE | E | 113 | 48.297 | −1.633 | −22.764 | 1.00 | 44.74 | E |
| ATOM | 15312 | CG2 | ILE | E | 113 | 48.516 | −0.241 | −22.191 | 1.00 | 40.76 | E |
| ATOM | 15313 | CG1 | ILE | E | 113 | 46.900 | −2.140 | −22.412 | 1.00 | 41.80 | E |
| ATOM | 15314 | CD1 | ILE | E | 113 | 46.600 | −2.080 | −20.910 | 1.00 | 42.28 | E |
| ATOM | 15315 | C | ILE | E | 113 | 49.885 | −1.041 | −24.533 | 1.00 | 65.52 | E |
| ATOM | 15316 | O | ILE | E | 113 | 50.869 | −1.726 | −24.264 | 1.00 | 68.92 | E |
| ATOM | 15317 | N | TYR | E | 114 | 49.976 | 0.180 | −25.048 | 1.00 | 43.28 | E |
| ATOM | 15318 | CA | TYR | E | 114 | 51.273 | 0.781 | −25.324 | 1.00 | 41.56 | E |
| ATOM | 15319 | CB | TYR | E | 114 | 51.368 | 1.241 | −26.789 | 1.00 | 33.45 | E |
| ATOM | 15320 | CG | TYR | E | 114 | 52.757 | 1.671 | −27.205 | 1.00 | 31.31 | E |
| ATOM | 15321 | CD1 | TYR | E | 114 | 53.709 | 0.727 | −27.595 | 1.00 | 35.60 | E |
| ATOM | 15322 | CE1 | TYR | E | 114 | 55.015 | 1.101 | −27.915 | 1.00 | 35.17 | E |
| ATOM | 15323 | CD2 | TYR | E | 114 | 53.141 | 3.016 | −27.149 | 1.00 | 31.48 | E |
| ATOM | 15324 | CE2 | TYR | E | 114 | 54.436 | 3.406 | −27.467 | 1.00 | 35.81 | E |
| ATOM | 15325 | CZ | TYR | E | 114 | 55.376 | 2.438 | −27.851 | 1.00 | 35.89 | E |
| ATOM | 15326 | OH | TYR | E | 114 | 56.674 | 2.794 | −28.173 | 1.00 | 35.39 | E |
| ATOM | 15327 | C | TYR | E | 114 | 51.488 | 1.972 | −24.404 | 1.00 | 41.40 | E |
| ATOM | 15328 | O | TYR | E | 114 | 50.552 | 2.712 | −24.105 | 1.00 | 44.31 | E |
| ATOM | 15329 | N | LEU | E | 115 | 52.729 | 2.154 | −23.972 | 1.00 | 49.47 | E |
| ATOM | 15330 | CA | LEU | E | 115 | 53.095 | 3.238 | −23.072 | 1.00 | 48.51 | E |
| ATOM | 15331 | CB | LEU | E | 115 | 53.244 | 2.680 | −21.653 | 1.00 | 73.89 | E |
| ATOM | 15332 | CG | LEU | E | 115 | 52.235 | 3.019 | −20.551 | 1.00 | 77.14 | E |
| ATOM | 15333 | CD1 | LEU | E | 115 | 50.822 | 3.076 | −21.092 | 1.00 | 75.52 | E |
| ATOM | 15334 | CD2 | LEU | E | 115 | 52.361 | 1.980 | −19.450 | 1.00 | 80.44 | E |
| ATOM | 15335 | C | LEU | E | 115 | 54.425 | 3.838 | −23.528 | 1.00 | 50.95 | E |
| ATOM | 15336 | O | LEU | E | 115 | 55.342 | 3.107 | −23.897 | 1.00 | 53.48 | E |
| ATOM | 15337 | N | GLY | E | 116 | 54.543 | 5.159 | −23.498 | 1.00 | 44.99 | E |
| ATOM | 15338 | CA | GLY | E | 116 | 55.793 | 5.763 | −23.916 | 1.00 | 39.33 | E |
| ATOM | 15339 | C | GLY | E | 116 | 55.873 | 7.257 | −23.709 | 1.00 | 43.18 | E |
| ATOM | 15340 | O | GLY | E | 116 | 54.898 | 7.971 | −23.927 | 1.00 | 42.06 | E |
| ATOM | 15341 | N | GLY | E | 117 | 57.043 | 7.724 | −23.287 | 1.00 | 40.89 | E |
| ATOM | 15342 | CA | GLY | E | 117 | 57.248 | 9.142 | −23.053 | 1.00 | 43.89 | E |
| ATOM | 15343 | C | GLY | E | 117 | 58.713 | 9.455 | −22.774 | 1.00 | 45.20 | E |
| ATOM | 15344 | O | GLY | E | 117 | 59.519 | 8.540 | −22.577 | 1.00 | 46.58 | E |
| ATOM | 15345 | N | VAL | E | 118 | 59.061 | 10.742 | −22.757 | 1.00 | 44.78 | E |
| ATOM | 15346 | CA | VAL | E | 118 | 60.436 | 11.174 | −22.503 | 1.00 | 47.56 | E |
| ATOM | 15347 | CB | VAL | E | 118 | 60.835 | 12.279 | −23.483 | 1.00 | 36.69 | E |
| ATOM | 15348 | CG1 | VAL | E | 118 | 62.228 | 12.783 | −23.165 | 1.00 | 38.09 | E |
| ATOM | 15349 | CG2 | VAL | E | 118 | 60.776 | 11.744 | −24.903 | 1.00 | 36.75 | E |
| ATOM | 15350 | C | VAL | E | 118 | 60.580 | 11.673 | −21.065 | 1.00 | 45.47 | E |
| ATOM | 15351 | O | VAL | E | 118 | 59.719 | 12.393 | −20.577 | 1.00 | 49.42 | E |
| ATOM | 15352 | N | PHE | E | 119 | 61.660 | 11.281 | −20.388 | 1.00 | 48.03 | E |
| ATOM | 15353 | CA | PHE | E | 119 | 61.869 | 11.662 | −18.990 | 1.00 | 49.34 | E |
| ATOM | 15354 | CB | PHE | E | 119 | 61.372 | 10.559 | −18.056 | 1.00 | 34.83 | E |
| ATOM | 15355 | CG | PHE | E | 119 | 59.904 | 10.273 | −18.179 | 1.00 | 32.43 | E |
| ATOM | 15356 | CD1 | PHE | E | 119 | 59.414 | 9.447 | −19.187 | 1.00 | 30.62 | E |
| ATOM | 15357 | CD2 | PHE | E | 119 | 58.995 | 10.855 | −17.305 | 1.00 | 31.11 | E |
| ATOM | 15358 | CE1 | PHE | E | 119 | 58.033 | 9.212 | −19.318 | 1.00 | 31.69 | E |
| ATOM | 15359 | CE2 | PHE | E | 119 | 57.620 | 10.624 | −17.428 | 1.00 | 31.92 | E |
| ATOM | 15360 | CZ | PHE | E | 119 | 57.140 | 9.807 | −18.430 | 1.00 | 31.01 | E |
| ATOM | 15361 | C | PHE | E | 119 | 63.309 | 11.973 | −18.613 | 1.00 | 50.25 | E |
| ATOM | 15362 | O | PHE | E | 119 | 64.250 | 11.434 | −19.201 | 1.00 | 46.35 | E |
| ATOM | 15363 | N | GLN | E | 120 | 63.460 | 12.850 | −17.620 | 1.00 | 41.40 | E |
| ATOM | 15364 | CA | GLN | E | 120 | 64.764 | 13.247 | −17.093 | 1.00 | 44.77 | E |
| ATOM | 15365 | CB | GLN | E | 120 | 64.670 | 14.611 | −16.412 | 1.00 | 84.23 | E |
| ATOM | 15366 | CG | GLN | E | 120 | 65.581 | 15.686 | −16.985 | 1.00 | 84.38 | E |
| ATOM | 15367 | CD | GLN | E | 120 | 67.038 | 15.262 | −17.039 | 1.00 | 83.98 | E |
| ATOM | 15368 | OE1 | GLN | E | 120 | 67.542 | 14.629 | −16.116 | 1.00 | 83.54 | E |
| ATOM | 15369 | NE2 | GLN | E | 120 | 67.727 | 15.624 | −18.118 | 1.00 | 86.81 | E |
| ATOM | 15370 | C | GLN | E | 120 | 65.087 | 12.187 | −16.046 | 1.00 | 43.91 | E |
| ATOM | 15371 | O | GLN | E | 120 | 64.302 | 11.987 | −15.129 | 1.00 | 46.61 | E |
| ATOM | 15372 | N | LEU | E | 121 | 66.223 | 11.502 | −16.186 | 1.00 | 46.31 | E |
| ATOM | 15373 | CA | LEU | E | 121 | 66.615 | 10.449 | −15.242 | 1.00 | 47.97 | E |
| ATOM | 15374 | CB | LEU | E | 121 | 66.591 | 9.092 | −15.939 | 1.00 | 46.08 | E |
| ATOM | 15375 | CG | LEU | E | 121 | 65.310 | 8.270 | −15.897 | 1.00 | 44.35 | E |
| ATOM | 15376 | CD1 | LEU | E | 121 | 64.107 | 9.163 | −15.796 | 1.00 | 45.74 | E |
| ATOM | 15377 | CD2 | LEU | E | 121 | 65.242 | 7.383 | −17.133 | 1.00 | 39.84 | E |
| ATOM | 15378 | C | LEU | E | 121 | 68.001 | 10.668 | −14.652 | 1.00 | 50.01 | E |
| ATOM | 15379 | O | LEU | E | 121 | 68.876 | 11.247 | −15.305 | 1.00 | 55.28 | E |
| ATOM | 15380 | N | GLU | E | 122 | 68.189 | 10.195 | −13.420 | 1.00 | 63.26 | E |
| ATOM | 15381 | CA | GLU | E | 122 | 69.461 | 10.313 | −12.697 | 1.00 | 64.80 | E |
| ATOM | 15382 | CB | GLU | E | 122 | 69.242 | 10.492 | −11.194 | 1.00 | 100.81 | E |
| ATOM | 15383 | CG | GLU | E | 122 | 68.474 | 11.699 | −10.724 | 1.00 | 103.85 | E |
| ATOM | 15384 | CD | GLU | E | 122 | 68.255 | 11.655 | −9.211 | 1.00 | 109.13 | E |
| ATOM | 15385 | OE1 | GLU | E | 122 | 67.585 | 10.714 | −8.728 | 1.00 | 110.63 | E |
| ATOM | 15386 | OE2 | GLU | E | 122 | 68.760 | 12.553 | −8.502 | 1.00 | 109.22 | E |
| ATOM | 15387 | C | GLU | E | 122 | 70.252 | 9.027 | −12.841 | 1.00 | 63.60 | E |
| ATOM | 15388 | O | GLU | E | 122 | 69.669 | 7.952 | −12.959 | 1.00 | 64.79 | E |
| ATOM | 15389 | N | LYS | E | 123 | 71.575 | 9.124 | −12.805 | 1.00 | 61.38 | E |
| ATOM | 15390 | CA | LYS | E | 123 | 72.391 | 7.925 | −12.873 | 1.00 | 62.23 | E |
| ATOM | 15391 | CB | LYS | E | 123 | 73.869 | 8.271 | −12.755 | 1.00 | 81.87 | E |
| ATOM | 15392 | CG | LYS | E | 123 | 74.753 | 7.054 | −12.588 | 1.00 | 78.03 | E |
| ATOM | 15393 | CD | LYS | E | 123 | 76.201 | 7.447 | −12.408 | 1.00 | 75.70 | E |
| ATOM | 15394 | CE | LYS | E | 123 | 77.070 | 6.223 | −12.215 | 1.00 | 77.43 | E |
| ATOM | 15395 | NZ | LYS | E | 123 | 78.489 | 6.603 | −11.951 | 1.00 | 81.44 | E |
| ATOM | 15396 | C | LYS | E | 123 | 71.979 | 7.058 | −11.681 | 1.00 | 63.05 | E |
| ATOM | 15397 | O | LYS | E | 123 | 71.975 | 7.519 | −10.546 | 1.00 | 67.81 | E |
| ATOM | 15398 | N | GLY | E | 124 | 71.609 | 5.811 | −11.941 | 1.00 | 46.81 | E |
| ATOM | 15399 | CA | GLY | E | 124 | 71.210 | 4.927 | −10.867 | 1.00 | 47.92 | E |
| ATOM | 15400 | C | GLY | E | 124 | 69.717 | 4.658 | −10.761 | 1.00 | 49.33 | E |
| ATOM | 15401 | O | GLY | E | 124 | 69.300 | 3.744 | −10.040 | 1.00 | 50.13 | E |
| ATOM | 15402 | N | ASP | E | 125 | 68.901 | 5.435 | −11.469 | 1.00 | 48.84 | E |
| ATOM | 15403 | CA | ASP | E | 125 | 67.458 | 5.232 | −11.398 | 1.00 | 47.63 | E |
| ATOM | 15404 | CB | ASP | E | 125 | 66.722 | 6.288 | −12.220 | 1.00 | 98.88 | E |
| ATOM | 15405 | CG | ASP | E | 125 | 66.797 | 7.668 | −11.602 | 1.00 | 104.34 | E |
| ATOM | 15406 | OD1 | ASP | E | 125 | 67.214 | 7.784 | −10.432 | 1.00 | 105.99 | E |
| ATOM | 15407 | OD2 | ASP | E | 125 | 66.426 | 8.641 | −12.289 | 1.00 | 103.00 | E |
| ATOM | 15408 | C | ASP | E | 125 | 67.055 | 3.838 | −11.872 | 1.00 | 46.68 | E |
| ATOM | 15409 | O | ASP | E | 125 | 67.705 | 3.255 | −12.743 | 1.00 | 43.59 | E |
| ATOM | 15410 | N | ARG | E | 126 | 65.982 | 3.312 | −11.284 | 1.00 | 67.69 | E |
| ATOM | 15411 | CA | ARG | E | 126 | 65.460 | 1.995 | −11.625 | 1.00 | 63.17 | E |
| ATOM | 15412 | CB | ARG | E | 126 | 65.326 | 1.135 | −10.370 | 1.00 | 88.24 | E |
| ATOM | 15413 | CG | ARG | E | 126 | 66.629 | 0.698 | −9.743 | 1.00 | 86.73 | E |
| ATOM | 15414 | CD | ARG | E | 126 | 66.369 | 0.046 | −8.388 | 1.00 | 91.65 | E |
| ATOM | 15415 | NE | ARG | E | 126 | 67.534 | −0.689 | −7.903 | 1.00 | 95.20 | E |
| ATOM | 15416 | CZ | ARG | E | 126 | 67.893 | −1.898 | −8.335 | 1.00 | 98.43 | E |
| ATOM | 15417 | NH1 | ARG | E | 126 | 68.975 | −2.493 | −7.840 | 1.00 | 98.10 | E |
| ATOM | 15418 | NH2 | ARG | E | 126 | 67.161 | −2.525 | −9.249 | 1.00 | 96.62 | E |
| ATOM | 15419 | C | ARG | E | 126 | 64.081 | 2.121 | −12.278 | 1.00 | 64.01 | E |
| ATOM | 15420 | O | ARG | E | 126 | 63.161 | 2.700 | −11.695 | 1.00 | 61.23 | E |
| ATOM | 15421 | N | LEU | E | 127 | 63.929 | 1.568 | −13.478 | 1.00 | 57.63 | E |
| ATOM | 15422 | CA | LEU | E | 127 | 62.648 | 1.633 | −14.171 | 1.00 | 54.01 | E |
| ATOM | 15423 | CB | LEU | E | 127 | 62.857 | 2.125 | −15.605 | 1.00 | 49.30 | E |
| ATOM | 15424 | CG | LEU | E | 127 | 63.499 | 3.511 | −15.693 | 1.00 | 48.28 | E |
| ATOM | 15425 | CD1 | LEU | E | 127 | 63.984 | 3.773 | −17.102 | 1.00 | 48.86 | E |
| ATOM | 15426 | CD2 | LEU | E | 127 | 62.497 | 4.562 | −15.263 | 1.00 | 48.94 | E |
| ATOM | 15427 | C | LEU | E | 127 | 61.977 | 0.267 | −14.170 | 1.00 | 53.76 | E |
| ATOM | 15428 | O | LEU | E | 127 | 62.620 | −0.755 | −14.403 | 1.00 | 49.96 | E |
| ATOM | 15429 | N | SER | E | 128 | 60.678 | 0.259 | −13.908 | 1.00 | 47.58 | E |
| ATOM | 15430 | CA | SER | E | 128 | 59.909 | −0.977 | −13.853 | 1.00 | 47.25 | E |
| ATOM | 15431 | CB | SER | E | 128 | 59.357 | −1.194 | −12.439 | 1.00 | 73.27 | E |
| ATOM | 15432 | OG | SER | E | 128 | 60.300 | −0.831 | −11.446 | 1.00 | 79.12 | E |
| ATOM | 15433 | C | SER | E | 128 | 58.734 | −0.924 | −14.811 | 1.00 | 46.43 | E |
| ATOM | 15434 | O | SER | E | 128 | 58.081 | 0.111 | −14.941 | 1.00 | 47.71 | E |
| ATOM | 15435 | N | ALA | E | 129 | 58.467 | −2.042 | −15.472 | 1.00 | 50.59 | E |
| ATOM | 15436 | CA | ALA | E | 129 | 57.342 | −2.147 | −16.392 | 1.00 | 51.56 | E |
| ATOM | 15437 | CB | ALA | E | 129 | 57.838 | −2.430 | −17.807 | 1.00 | 49.59 | E |
| ATOM | 15438 | C | ALA | E | 129 | 56.532 | −3.322 | −15.857 | 1.00 | 50.33 | E |
| ATOM | 15439 | O | ALA | E | 129 | 56.868 | −4.484 | −16.108 | 1.00 | 51.21 | E |
| ATOM | 15440 | N | GLU | E | 130 | 55.469 | −3.022 | −15.113 | 1.00 | 50.18 | E |
| ATOM | 15441 | CA | GLU | E | 130 | 54.656 | −4.064 | −14.502 | 1.00 | 51.15 | E |
| ATOM | 15442 | CB | GLU | E | 130 | 54.735 | −3.921 | −12.985 | 1.00 | 114.97 | E |
| ATOM | 15443 | CG | GLU | E | 130 | 56.160 | −4.014 | −12.456 | 1.00 | 114.51 | E |
| ATOM | 15444 | CD | GLU | E | 130 | 56.333 | −3.389 | −11.081 | 1.00 | 120.00 | E |
| ATOM | 15445 | OE1 | GLU | E | 130 | 57.451 | −3.478 | −10.527 | 1.00 | 121.37 | E |
| ATOM | 15446 | OE2 | GLU | E | 130 | 55.360 | −2.806 | −10.554 | 1.00 | 119.81 | E |
| ATOM | 15447 | C | GLU | E | 130 | 53.208 | −4.099 | −14.947 | 1.00 | 47.54 | E |
| ATOM | 15448 | O | GLU | E | 130 | 52.707 | −3.146 | −15.533 | 1.00 | 47.46 | E |
| ATOM | 15449 | N | ILE | E | 131 | 52.554 | −5.231 | −14.686 | 1.00 | 75.14 | E |
| ATOM | 15450 | CA | ILE | E | 131 | 51.142 | −5.435 | −15.018 | 1.00 | 77.68 | E |
| ATOM | 15451 | CB | ILE | E | 131 | 50.949 | −6.401 | −16.199 | 1.00 | 69.01 | E |
| ATOM | 15452 | CG2 | ILE | E | 131 | 51.485 | −5.774 | −17.477 | 1.00 | 69.24 | E |
| ATOM | 15453 | CG1 | ILE | E | 131 | 51.632 | −7.735 | −15.890 | 1.00 | 65.20 | E |
| ATOM | 15454 | CD1 | ILE | E | 131 | 51.418 | −8.780 | −16.951 | 1.00 | 63.81 | E |
| ATOM | 15455 | C | ILE | E | 131 | 50.490 | −6.059 | −13.800 | 1.00 | 79.57 | E |
| ATOM | 15456 | O | ILE | E | 131 | 51.177 | −6.615 | −12.945 | 1.00 | 80.29 | E |
| ATOM | 15457 | N | ASN | E | 132 | 49.168 | −5.989 | −13.728 | 1.00 | 67.58 | E |
| ATOM | 15458 | CA | ASN | E | 132 | 48.459 | −6.532 | −12.583 | 1.00 | 64.58 | E |
| ATOM | 15459 | CB | ASN | E | 132 | 47.284 | −5.628 | −12.246 | 1.00 | 65.53 | E |
| ATOM | 15460 | CG | ASN | E | 132 | 46.238 | −5.628 | −13.330 | 1.00 | 64.23 | E |
| ATOM | 15461 | OD1 | ASN | E | 132 | 46.567 | −5.611 | −14.510 | 1.00 | 65.92 | E |
| ATOM | 15462 | ND2 | ASN | E | 132 | 44.971 | −5.647 | −12.942 | 1.00 | 62.06 | E |
| ATOM | 15463 | C | ASN | E | 132 | 47.962 | −7.965 | −12.773 | 1.00 | 62.23 | E |
| ATOM | 15464 | O | ASN | E | 132 | 47.825 | −8.702 | −11.802 | 1.00 | 65.40 | E |
| ATOM | 15465 | N | ARG | E | 133 | 47.691 | −8.363 | −14.014 | 1.00 | 62.53 | E |
| ATOM | 15466 | CA | ARG | E | 133 | 47.199 | −9.714 | −14.289 | 1.00 | 58.12 | E |
| ATOM | 15467 | CB | ARG | E | 133 | 45.804 | −9.645 | −14.926 | 1.00 | 71.31 | E |
| ATOM | 15468 | CG | ARG | E | 133 | 44.743 | −9.004 | −14.049 | 1.00 | 73.89 | E |
| ATOM | 15469 | CD | ARG | E | 133 | 44.623 | −9.735 | −12.712 | 1.00 | 79.13 | E |
| ATOM | 15470 | NE | ARG | E | 133 | 44.266 | −11.144 | −12.875 | 1.00 | 76.99 | E |
| ATOM | 15471 | CZ | ARG | E | 133 | 43.069 | −11.578 | −13.258 | 1.00 | 78.09 | E |
| ATOM | 15472 | NH1 | ARG | E | 133 | 42.848 | −12.880 | −13.382 | 1.00 | 80.77 | E |
| ATOM | 15473 | NH2 | ARG | E | 133 | 42.093 | −10.712 | −13.508 | 1.00 | 77.38 | E |
| ATOM | 15474 | C | ARG | E | 133 | 48.121 | −10.533 | −15.190 | 1.00 | 62.71 | E |
| ATOM | 15475 | O | ARG | E | 133 | 47.764 | −10.850 | −16.322 | 1.00 | 65.07 | E |
| ATOM | 15476 | N | PRO | E | 134 | 49.310 | −10.906 | −14.689 | 1.00 | 67.72 | E |
| ATOM | 15477 | CD | PRO | E | 134 | 49.789 | −10.733 | −13.310 | 1.00 | 87.54 | E |
| ATOM | 15478 | CA | PRO | E | 134 | 50.271 | −11.693 | −15.466 | 1.00 | 71.44 | E |
| ATOM | 15479 | CB | PRO | E | 134 | 51.444 | −11.858 | −14.503 | 1.00 | 90.26 | E |
| ATOM | 15480 | CG | PRO | E | 134 | 50.778 | −11.875 | −13.180 | 1.00 | 92.26 | E |
| ATOM | 15481 | C | PRO | E | 134 | 49.715 | −13.032 | −15.932 | 1.00 | 76.62 | E |
| ATOM | 15482 | O | PRO | E | 134 | 50.320 | −13.709 | −16.756 | 1.00 | 79.66 | E |
| ATOM | 15483 | N | ASP | E | 135 | 48.564 | −13.421 | −15.404 | 1.00 | 87.75 | E |
| ATOM | 15484 | CA | ASP | E | 135 | 47.963 | −14.681 | −15.807 | 1.00 | 84.21 | E |
| ATOM | 15485 | CB | ASP | E | 135 | 46.963 | −15.147 | −14.744 | 1.00 | 101.10 | E |
| ATOM | 15486 | CG | ASP | E | 135 | 46.101 | −14.019 | −14.219 | 1.00 | 100.57 | E |
| ATOM | 15487 | OD1 | ASP | E | 135 | 45.183 | −13.579 | −14.942 | 1.00 | 96.51 | E |
| ATOM | 15488 | OD2 | ASP | E | 135 | 46.349 | −13.566 | −13.081 | 1.00 | 102.15 | E |
| ATOM | 15489 | C | ASP | E | 135 | 47.291 | −14.554 | −17.177 | 1.00 | 82.92 | E |
| ATOM | 15490 | O | ASP | E | 135 | 46.931 | −15.557 | −17.793 | 1.00 | 89.61 | E |
| ATOM | 15491 | N | TYR | E | 136 | 47.138 | −13.319 | −17.651 | 1.00 | 68.64 | E |
| ATOM | 15492 | CA | TYR | E | 136 | 46.524 | −13.059 | −18.955 | 1.00 | 69.85 | E |
| ATOM | 15493 | CB | TYR | E | 136 | 45.484 | −11.943 | −18.848 | 1.00 | 84.01 | E |
| ATOM | 15494 | CG | TYR | E | 136 | 44.183 | −12.338 | −18.196 | 1.00 | 83.62 | E |
| ATOM | 15495 | CD1 | TYR | E | 136 | 43.731 | −11.680 | −17.056 | 1.00 | 83.22 | E |
| ATOM | 15496 | CE1 | TYR | E | 136 | 42.510 | −11.994 | −16.475 | 1.00 | 85.77 | E |
| ATOM | 15497 | CD2 | TYR | E | 136 | 43.377 | −13.334 | −18.744 | 1.00 | 85.79 | E |
| ATOM | 15498 | CE2 | TYR | E | 136 | 42.151 | −13.658 | −18.169 | 1.00 | 85.26 | E |
| ATOM | 15499 | CZ | TYR | E | 136 | 41.725 | −12.979 | −17.033 | 1.00 | 86.56 | E |
| ATOM | 15500 | OH | TYR | E | 136 | 40.512 | −13.280 | −16.457 | 1.00 | 90.40 | E |
| ATOM | 15501 | C | TYR | E | 136 | 47.575 | −12.650 | −19.990 | 1.00 | 71.76 | E |
| ATOM | 15502 | O | TYR | E | 136 | 47.251 | −12.318 | −21.127 | 1.00 | 74.64 | E |
| ATOM | 15503 | N | LEU | E | 137 | 48.835 | −12.681 | −19.587 | 1.00 | 89.04 | E |
| ATOM | 15504 | CA | LEU | E | 137 | 49.926 | −12.298 | −20.466 | 1.00 | 85.75 | E |
| ATOM | 15505 | CB | LEU | E | 137 | 51.233 | −12.297 | −19.686 | 1.00 | 77.93 | E |
| ATOM | 15506 | CG | LEU | E | 137 | 51.985 | −10.979 | −19.593 | 1.00 | 75.66 | E |
| ATOM | 15507 | CD1 | LEU | E | 137 | 53.355 | −11.252 | −19.013 | 1.00 | 77.80 | E |
| ATOM | 15508 | CD2 | LEU | E | 137 | 52.118 | −10.350 | −20.959 | 1.00 | 79.42 | E |
| ATOM | 15509 | C | LEU | E | 137 | 50.107 | −13.197 | −21.679 | 1.00 | 85.12 | E |
| ATOM | 15510 | O | LEU | E | 137 | 50.424 | −14.376 | −21.531 | 1.00 | 83.97 | E |
| ATOM | 15511 | N | LEU | E | 138 | 49.922 | −12.647 | −22.879 | 1.00 | 89.47 | E |
| ATOM | 15512 | CA | LEU | E | 138 | 50.125 | −13.432 | −24.095 | 1.00 | 87.27 | E |
| ATOM | 15513 | CB | LEU | E | 138 | 49.045 | −13.165 | −25.156 | 1.00 | 69.33 | E |
| ATOM | 15514 | CG | LEU | E | 138 | 49.419 | −13.762 | −26.530 | 1.00 | 65.45 | E |
| ATOM | 15515 | CD1 | LEU | E | 138 | 49.850 | −15.216 | −26.371 | 1.00 | 67.02 | E |
| ATOM | 15516 | CD2 | LEU | E | 138 | 48.256 | −13.663 | −27.495 | 1.00 | 68.37 | E |
| ATOM | 15517 | C | LEU | E | 138 | 51.487 | −13.132 | −24.703 | 1.00 | 90.62 | E |
| ATOM | 15518 | O | LEU | E | 138 | 51.651 | −12.156 | −25.431 | 1.00 | 89.77 | E |
| ATOM | 15519 | N | PHE | E | 139 | 52.464 | −13.971 | −24.392 | 1.00 | 65.08 | E |
| ATOM | 15520 | CA | PHE | E | 139 | 53.801 | −13.806 | −24.936 | 1.00 | 66.46 | E |
| ATOM | 15521 | CB | PHE | E | 139 | 54.815 | −13.626 | −23.803 | 1.00 | 104.94 | E |
| ATOM | 15522 | CG | PHE | E | 139 | 54.633 | −14.580 | −22.660 | 1.00 | 104.09 | E |
| ATOM | 15523 | CD1 | PHE | E | 139 | 55.019 | −14.209 | −21.378 | 1.00 | 103.62 | E |
| ATOM | 15524 | CD2 | PHE | E | 139 | 54.099 | −15.850 | −22.860 | 1.00 | 106.70 | E |
| ATOM | 15525 | CE1 | PHE | E | 139 | 54.878 | −15.085 | −20.310 | 1.00 | 108.26 | E |
| ATOM | 15526 | CE2 | PHE | E | 139 | 53.953 | −16.734 | −21.802 | 1.00 | 104.24 | E |
| ATOM | 15527 | CZ | PHE | E | 139 | 54.343 | −16.351 | −20.522 | 1.00 | 108.18 | E |
| ATOM | 15528 | C | PHE | E | 139 | 54.140 | −15.022 | −25.796 | 1.00 | 70.49 | E |
| ATOM | 15529 | O | PHE | E | 139 | 55.272 | −15.505 | −25.801 | 1.00 | 70.59 | E |
| ATOM | 15530 | N | ALA | E | 140 | 53.135 | −15.498 | −26.528 | 1.00 | 81.82 | E |
| ATOM | 15531 | CA | ALA | E | 140 | 53.277 | −16.664 | −27.393 | 1.00 | 83.12 | E |
| ATOM | 15532 | CB | ALA | E | 140 | 51.903 | −17.275 | −27.684 | 1.00 | 56.26 | E |
| ATOM | 15533 | C | ALA | E | 140 | 53.979 | −16.354 | −28.703 | 1.00 | 83.51 | E |
| ATOM | 15534 | O | ALA | E | 140 | 54.878 | −17.086 | −29.112 | 1.00 | 85.40 | E |
| ATOM | 15535 | N | GLU | E | 141 | 53.566 | −15.274 | −29.360 | 1.00 | 108.89 | E |
| ATOM | 15536 | CA | GLU | E | 141 | 54.152 | −14.906 | −30.638 | 1.00 | 112.07 | E |
| ATOM | 15537 | CB | GLU | E | 141 | 53.301 | −13.841 | −31.337 | 1.00 | 177.34 | E |
| ATOM | 15538 | CG | GLU | E | 141 | 53.762 | −13.505 | −32.754 | 1.00 | 177.34 | E |
| ATOM | 15539 | CD | GLU | E | 141 | 54.870 | −12.463 | −32.796 | 1.00 | 177.34 | E |
| ATOM | 15540 | OE1 | GLU | E | 141 | 55.638 | −12.453 | −33.778 | 1.00 | 177.34 | E |
| ATOM | 15541 | OE2 | GLU | E | 141 | 54.967 | −11.642 | −31.859 | 1.00 | 177.34 | E |
| ATOM | 15542 | C | GLU | E | 141 | 55.596 | −14.439 | −30.547 | 1.00 | 114.17 | E |
| ATOM | 15543 | O | GLU | E | 141 | 56.508 | −15.228 | −30.775 | 1.00 | 117.85 | E |
| ATOM | 15544 | N | SER | E | 142 | 55.824 | −13.175 | −30.209 | 1.00 | 93.92 | E |
| ATOM | 15545 | CA | SER | E | 142 | 57.196 | −12.689 | −30.159 | 1.00 | 96.91 | E |
| ATOM | 15546 | CB | SER | E | 142 | 57.854 | −12.912 | −31.516 | 1.00 | 55.33 | E |
| ATOM | 15547 | OG | SER | E | 142 | 59.261 | −12.796 | −31.434 | 1.00 | 56.21 | E |
| ATOM | 15548 | C | SER | E | 142 | 57.327 | −11.217 | −29.786 | 1.00 | 98.23 | E |
| ATOM | 15549 | O | SER | E | 142 | 57.666 | −10.878 | −28.644 | 1.00 | 103.32 | E |
| ATOM | 15550 | N | GLY | E | 143 | 57.091 | −10.338 | −30.756 | 1.00 | 49.78 | E |
| ATOM | 15551 | CA | GLY | E | 143 | 57.193 | −8.923 | −30.458 | 1.00 | 47.25 | E |
| ATOM | 15552 | C | GLY | E | 143 | 55.906 | −8.378 | −29.863 | 1.00 | 45.32 | E |
| ATOM | 15553 | O | GLY | E | 143 | 55.525 | −7.253 | −30.177 | 1.00 | 47.31 | E |
| ATOM | 15554 | N | GLN | E | 144 | 55.251 | −9.161 | −29.000 | 1.00 | 55.01 | E |
| ATOM | 15555 | CA | GLN | E | 144 | 53.987 | −8.764 | −28.377 | 1.00 | 49.54 | E |
| ATOM | 15556 | CB | GLN | E | 144 | 53.058 | −9.971 | −28.258 | 1.00 | 91.87 | E |
| ATOM | 15557 | CG | GLN | E | 144 | 52.955 | −10.805 | −29.516 | 1.00 | 91.15 | E |
| ATOM | 15558 | CD | GLN | E | 144 | 51.918 | −11.911 | −29.398 | 1.00 | 91.12 | E |
| ATOM | 15559 | OE1 | GLN | E | 144 | 51.906 | −12.669 | −28.428 | 1.00 | 96.69 | E |
| ATOM | 15560 | NE2 | GLN | E | 144 | 51.047 | −12.014 | −30.396 | 1.00 | 89.66 | E |
| ATOM | 15561 | C | GLN | E | 144 | 54.149 | −8.138 | −26.996 | 1.00 | 51.34 | E |
| ATOM | 15562 | O | GLN | E | 144 | 53.271 | −7.419 | −26.529 | 1.00 | 53.65 | E |
| ATOM | 15563 | N | VAL | E | 145 | 55.270 | −8.415 | −26.340 | 1.00 | 41.00 | E |
| ATOM | 15564 | CA | VAL | E | 145 | 55.531 | −7.884 | −25.009 | 1.00 | 39.25 | E |
| ATOM | 15565 | CB | VAL | E | 145 | 55.288 | −8.962 | −23.934 | 1.00 | 40.03 | E |
| ATOM | 15566 | CG1 | VAL | E | 145 | 55.547 | −8.401 | −22.559 | 1.00 | 38.19 | E |
| ATOM | 15567 | CG2 | VAL | E | 145 | 53.871 | −9.467 | −24.025 | 1.00 | 37.11 | E |
| ATOM | 15568 | C | VAL | E | 145 | 56.977 | −7.416 | −24.925 | 1.00 | 40.36 | E |
| ATOM | 15569 | O | VAL | E | 145 | 57.905 | −8.220 | −24.991 | 1.00 | 43.04 | E |
| ATOM | 15570 | N | TYR | E | 146 | 57.169 | −6.110 | −24.786 | 1.00 | 45.74 | E |
| ATOM | 15571 | CA | TYR | E | 146 | 58.508 | −5.555 | −24.699 | 1.00 | 43.95 | E |
| ATOM | 15572 | CB | TYR | E | 146 | 59.048 | −5.215 | −26.100 | 1.00 | 48.95 | E |
| ATOM | 15573 | CG | TYR | E | 146 | 58.102 | −4.422 | −26.958 | 1.00 | 48.98 | E |
| ATOM | 15574 | CD1 | TYR | E | 146 | 57.108 | −5.056 | −27.703 | 1.00 | 48.37 | E |
| ATOM | 15575 | CE1 | TYR | E | 146 | 56.191 | −4.325 | −28.462 | 1.00 | 47.16 | E |
| ATOM | 15576 | CD2 | TYR | E | 146 | 58.166 | −3.028 | −26.993 | 1.00 | 44.57 | E |
| ATOM | 15577 | CE2 | TYR | E | 146 | 57.252 | −2.280 | −27.747 | 1.00 | 46.62 | E |
| ATOM | 15578 | CZ | TYR | E | 146 | 56.265 | −2.936 | −28.477 | 1.00 | 46.66 | E |
| ATOM | 15579 | OH | TYR | E | 146 | 55.347 | −2.206 | −29.202 | 1.00 | 47.64 | E |
| ATOM | 15580 | C | TYR | E | 146 | 58.566 | −4.324 | −23.802 | 1.00 | 42.26 | E |
| ATOM | 15581 | O | TYR | E | 146 | 57.543 | −3.723 | −23.455 | 1.00 | 40.43 | E |
| ATOM | 15582 | N | PHE | E | 147 | 59.790 | −3.956 | −23.451 | 1.00 | 47.38 | E |
| ATOM | 15583 | CA | PHE | E | 147 | 60.052 | −2.832 | −22.577 | 1.00 | 46.90 | E |
| ATOM | 15584 | CB | PHE | E | 147 | 60.005 | −3.352 | −21.140 | 1.00 | 59.99 | E |
| ATOM | 15585 | CG | PHE | E | 147 | 60.500 | −2.388 | −20.102 | 1.00 | 59.04 | E |
| ATOM | 15586 | CD1 | PHE | E | 147 | 60.238 | −1.025 | −20.197 | 1.00 | 59.53 | E |
| ATOM | 15587 | CD2 | PHE | E | 147 | 61.169 | −2.864 | −18.974 | 1.00 | 61.37 | E |
| ATOM | 15588 | CE1 | PHE | E | 147 | 60.632 | −0.155 | −19.177 | 1.00 | 59.86 | E |
| ATOM | 15589 | CE2 | PHE | E | 147 | 61.563 | −2.003 | −17.954 | 1.00 | 59.94 | E |
| ATOM | 15590 | CZ | PHE | E | 147 | 61.292 | −0.646 | −18.056 | 1.00 | 60.78 | E |
| ATOM | 15591 | C | PHE | E | 147 | 61.439 | −2.316 | −22.954 | 1.00 | 48.44 | E |
| ATOM | 15592 | O | PHE | E | 147 | 62.391 | −3.093 | −23.041 | 1.00 | 45.92 | E |
| ATOM | 15593 | N | GLY | E | 148 | 61.558 | −1.015 | −23.204 | 1.00 | 32.34 | E |
| ATOM | 15594 | CA | GLY | E | 148 | 62.856 | −0.487 | −23.577 | 1.00 | 36.82 | E |
| ATOM | 15595 | C | GLY | E | 148 | 63.056 | 0.982 | −23.264 | 1.00 | 39.04 | E |
| ATOM | 15596 | O | GLY | E | 148 | 62.123 | 1.689 | −22.884 | 1.00 | 41.65 | E |
| ATOM | 15597 | N | ILE | E | 149 | 64.299 | 1.427 | −23.407 | 1.00 | 38.46 | E |
| ATOM | 15598 | CA | ILE | E | 149 | 64.652 | 2.818 | −23.186 | 1.00 | 36.87 | E |
| ATOM | 15599 | CB | ILE | E | 149 | 64.980 | 3.127 | −21.705 | 1.00 | 47.98 | E |
| ATOM | 15600 | CG2 | ILE | E | 149 | 63.868 | 2.603 | −20.798 | 1.00 | 48.53 | E |
| ATOM | 15601 | CG1 | ILE | E | 149 | 66.295 | 2.470 | −21.301 | 1.00 | 44.02 | E |
| ATOM | 15602 | CD1 | ILE | E | 149 | 66.715 | 2.859 | −19.929 | 1.00 | 47.27 | E |
| ATOM | 15603 | C | ILE | E | 149 | 65.874 | 3.144 | −24.025 | 1.00 | 38.91 | E |
| ATOM | 15604 | O | ILE | E | 149 | 66.723 | 2.285 | −24.271 | 1.00 | 44.08 | E |
| ATOM | 15605 | N | ILE | E | 150 | 65.951 | 4.391 | −24.463 | 1.00 | 50.05 | E |
| ATOM | 15606 | CA | ILE | E | 150 | 67.057 | 4.861 | −25.278 | 1.00 | 50.56 | E |
| ATOM | 15607 | CB | ILE | E | 150 | 66.651 | 4.888 | −26.789 | 1.00 | 41.12 | E |
| ATOM | 15608 | CG2 | ILE | E | 150 | 65.385 | 5.704 | −26.981 | 1.00 | 44.42 | E |
| ATOM | 15609 | CG1 | ILE | E | 150 | 67.773 | 5.474 | −27.647 | 1.00 | 39.11 | E |
| ATOM | 15610 | CD1 | ILE | E | 150 | 68.986 | 4.595 | −27.771 | 1.00 | 37.12 | E |
| ATOM | 15611 | C | ILE | E | 150 | 67.404 | 6.269 | −24.786 | 1.00 | 51.90 | E |
| ATOM | 15612 | O | ILE | E | 150 | 66.520 | 7.080 | −24.512 | 1.00 | 51.01 | E |
| ATOM | 15613 | N | ALA | E | 151 | 68.692 | 6.548 | −24.650 | 1.00 | 61.56 | E |
| ATOM | 15614 | CA | ALA | E | 151 | 69.126 | 7.858 | −24.191 | 1.00 | 60.30 | E |
| ATOM | 15615 | CB | ALA | E | 151 | 70.527 | 7.767 | −23.603 | 1.00 | 34.99 | E |
| ATOM | 15616 | C | ALA | E | 151 | 69.110 | 8.853 | −25.343 | 1.00 | 61.26 | E |
| ATOM | 15617 | O | ALA | E | 151 | 69.768 | 8.645 | −26.361 | 1.00 | 63.64 | E |
| ATOM | 15618 | N | LEU | E | 152 | 68.363 | 9.938 | −25.178 | 1.00 | 76.47 | E |
| ATOM | 15619 | CA | LEU | E | 152 | 68.277 | 10.948 | −26.217 | 1.00 | 80.45 | E |
| ATOM | 15620 | CB | LEU | E | 152 | 66.824 | 11.367 | −26.413 | 1.00 | 72.16 | E |
| ATOM | 15621 | CG | LEU | E | 152 | 65.903 | 10.225 | −26.846 | 1.00 | 73.41 | E |
| ATOM | 15622 | CD1 | LEU | E | 152 | 64.479 | 10.723 | −26.959 | 1.00 | 71.10 | E |
| ATOM | 15623 | CD2 | LEU | E | 152 | 66.374 | 9.663 | −28.174 | 1.00 | 72.61 | E |
| ATOM | 15624 | C | LEU | E | 152 | 69.127 | 12.156 | −25.873 | 1.00 | 83.30 | E |
| ATOM | 15625 | O | LEU | E | 152 | 70.152 | 12.398 | −26.510 | 1.00 | 89.16 | E |
| ATOM | 15626 | N | ILE | E | 153 | 71.405 | −23.865 | −22.438 | 1.00 | 46.46 | E |
| ATOM | 15627 | CA | ILE | E | 153 | 71.072 | −24.718 | −23.567 | 1.00 | 46.46 | E |
| ATOM | 15628 | CB | ILE | E | 153 | 69.640 | −24.474 | −24.052 | 1.00 | 45.67 | E |
| ATOM | 15629 | CG2 | ILE | E | 153 | 68.655 | −24.990 | −23.024 | 1.00 | 42.45 | E |
| ATOM | 15630 | CG1 | ILE | E | 153 | 69.433 | −22.985 | −24.335 | 1.00 | 42.45 | E |
| ATOM | 15631 | CD1 | ILE | E | 153 | 68.033 | −22.661 | −24.826 | 1.00 | 42.45 | E |
| ATOM | 15632 | C | ILE | E | 153 | 72.020 | −24.526 | −24.738 | 1.00 | 46.46 | E |
| ATOM | 15633 | O | ILE | E | 153 | 72.642 | −23.478 | −24.893 | 1.00 | 46.46 | E |
| ATOM | 15634 | N | THR | E | 154 | 72.082 | −25.548 | −25.583 | 1.00 | 56.31 | E |
| ATOM | 15635 | CA | THR | E | 154 | 72.983 | −25.563 | −26.715 | 1.00 | 56.31 | E |
| ATOM | 15636 | CB | THR | E | 154 | 74.257 | −26.352 | −26.321 | 1.00 | 42.26 | E |
| ATOM | 15637 | OG1 | THR | E | 154 | 75.306 | −25.429 | −26.022 | 1.00 | 52.54 | E |
| ATOM | 15638 | CG2 | THR | E | 154 | 74.694 | −27.327 | −27.426 | 1.00 | 52.54 | E |
| ATOM | 15639 | C | THR | E | 154 | 72.401 | −26.181 | −27.975 | 1.00 | 56.31 | E |
| ATOM | 15640 | O | THR | E | 154 | 71.627 | −27.135 | −27.894 | 1.00 | 56.31 | E |
| ATOM | 15641 | N | LEU | E | 155 | 72.774 | −25.625 | −29.132 | 1.00 | 41.26 | E |
| ATOM | 15642 | CA | LEU | E | 155 | 72.361 | −26.154 | −30.434 | 1.00 | 41.26 | E |
| ATOM | 15643 | CB | LEU | E | 155 | 71.312 | −25.268 | −31.100 | 1.00 | 42.16 | E |
| ATOM | 15644 | CG | LEU | E | 155 | 70.783 | −25.845 | −32.431 | 1.00 | 38.74 | E |
| ATOM | 15645 | CD1 | LEU | E | 155 | 70.245 | −27.246 | −32.209 | 1.00 | 38.74 | E |
| ATOM | 15646 | CD2 | LEU | E | 155 | 69.684 | −24.957 | −33.010 | 1.00 | 38.74 | E |
| ATOM | 15647 | C | LEU | E | 155 | 73.619 | −26.227 | −31.300 | 1.00 | 41.26 | E |
| ATOM | 15648 | O | LEU | E | 155 | 74.232 | −25.209 | −31.605 | 1.00 | 41.26 | E |
| ATOM | 15649 | N | LYS | E | 156 | 74.020 | −27.438 | −31.675 | 1.00 | 38.44 | E |
| ATOM | 15650 | CA | LYS | E | 156 | 75.223 | −27.612 | −32.478 | 1.00 | 38.44 | E |
| ATOM | 15651 | CB | LYS | E | 156 | 76.304 | −28.310 | −31.655 | 1.00 | 44.39 | E |
| ATOM | 15652 | CG | LYS | E | 156 | 77.621 | −28.427 | −32.398 | 1.00 | 46.58 | E |
| ATOM | 15653 | CD | LYS | E | 156 | 78.732 | −29.074 | −31.568 | 1.00 | 46.58 | E |
| ATOM | 15654 | CE | LYS | E | 156 | 78.504 | −30.571 | −31.368 | 1.00 | 46.58 | E |
| ATOM | 15655 | NZ | LYS | E | 156 | 79.670 | −31.249 | −30.716 | 1.00 | 46.58 | E |
| ATOM | 15656 | C | LYS | E | 156 | 75.012 | −28.394 | −33.773 | 1.00 | 38.44 | E |
| ATOM | 15657 | O | LYS | E | 156 | 74.389 | −29.449 | −33.757 | 1.00 | 38.44 | E |
| ATOM | 15658 | N | TYR | E | 157 | 75.532 | −27.858 | −34.882 | 1.00 | 40.73 | E |
| ATOM | 15659 | CA | TYR | E | 157 | 75.464 | −28.486 | −36.207 | 1.00 | 40.73 | E |
| ATOM | 15660 | CB | TYR | E | 157 | 74.870 | −27.523 | −37.237 | 1.00 | 56.49 | E |
| ATOM | 15661 | CG | TYR | E | 157 | 73.365 | −27.358 | −37.153 | 1.00 | 38.83 | E |
| ATOM | 15662 | CD1 | TYR | E | 157 | 72.634 | −27.913 | −36.106 | 1.00 | 38.83 | E |
| ATOM | 15663 | CE1 | TYR | E | 157 | 71.253 | −27.737 | −36.014 | 1.00 | 38.83 | E |
| ATOM | 15664 | CD2 | TYR | E | 157 | 72.670 | −26.622 | −38.113 | 1.00 | 38.83 | E |
| ATOM | 15665 | CE2 | TYR | E | 157 | 71.295 | −26.447 | −38.031 | 1.00 | 38.83 | E |
| ATOM | 15666 | CZ | TYR | E | 157 | 70.592 | −27.006 | −36.979 | 1.00 | 38.83 | E |
| ATOM | 15667 | OH | TYR | E | 157 | 69.227 | −26.840 | −36.875 | 1.00 | 38.83 | E |
| ATOM | 15668 | C | TYR | E | 157 | 76.894 | −28.868 | −36.621 | 1.00 | 40.73 | E |
| ATOM | 15669 | O | TYR | E | 157 | 77.807 | −28.036 | −36.593 | 1.00 | 40.73 | E |
| ATOM | 15670 | N | ASN | E | 158 | 77.087 | −30.126 | −36.999 | 1.00 | 37.20 | E |
| ATOM | 15671 | CA | ASN | E | 158 | 78.401 | −30.623 | −37.384 | 1.00 | 37.20 | E |
| ATOM | 15672 | CB | ASN | E | 158 | 78.905 | −31.599 | −36.309 | 1.00 | 33.73 | E |
| ATOM | 15673 | CG | ASN | E | 158 | 80.301 | −32.150 | −36.599 | 1.00 | 44.43 | E |
| ATOM | 15674 | OD1 | ASN | E | 158 | 80.898 | −31.846 | −37.639 | 1.00 | 44.43 | E |
| ATOM | 15675 | ND2 | ASN | E | 158 | 80.812 | −32.964 | −35.671 | 1.00 | 44.43 | E |
| ATOM | 15676 | C | ASN | E | 158 | 78.331 | −31.324 | −38.736 | 1.00 | 37.20 | E |
| ATOM | 15677 | O | ASN | E | 158 | 77.663 | −32.353 | −38.871 | 1.00 | 37.20 | E |
| ATOM | 15678 | N | TYR | E | 159 | 78.993 | −30.753 | −39.741 | 1.00 | 41.45 | E |
| ATOM | 15679 | CA | TYR | E | 159 | 79.029 | −31.353 | −41.071 | 1.00 | 41.45 | E |
| ATOM | 15680 | CB | TYR | E | 159 | 78.754 | −30.331 | −42.162 | 1.00 | 27.66 | E |
| ATOM | 15681 | CG | TYR | E | 159 | 77.378 | −29.704 | −42.147 | 1.00 | 31.22 | E |
| ATOM | 15682 | CD1 | TYR | E | 159 | 77.084 | −28.632 | −41.308 | 1.00 | 31.22 | E |
| ATOM | 15683 | CE1 | TYR | E | 159 | 75.846 | −27.993 | −41.360 | 1.00 | 31.22 | E |
| ATOM | 15684 | CD2 | TYR | E | 159 | 76.391 | −30.133 | −43.034 | 1.00 | 31.22 | E |
| ATOM | 15685 | CE2 | TYR | E | 159 | 75.148 | −29.503 | −43.094 | 1.00 | 31.22 | E |
| ATOM | 15686 | CZ | TYR | E | 159 | 74.883 | −28.428 | −42.255 | 1.00 | 31.22 | E |
| ATOM | 15687 | OH | TYR | E | 159 | 73.668 | −27.774 | −42.325 | 1.00 | 31.22 | E |
| ATOM | 15688 | C | TYR | E | 159 | 80.435 | −31.856 | −41.267 | 1.00 | 41.45 | E |
| ATOM | 15689 | O | TYR | E | 159 | 81.379 | −31.103 | −41.080 | 1.00 | 41.45 | E |
| ATOM | 15690 | N | THR | E | 160 | 80.591 | −33.115 | −41.652 | 1.00 | 36.46 | E |
| ATOM | 15691 | CA | THR | E | 160 | 81.930 | −33.656 | −41.858 | 1.00 | 36.46 | E |
| ATOM | 15692 | CB | THR | E | 160 | 82.382 | −34.441 | −40.627 | 1.00 | 49.53 | E |
| ATOM | 15693 | OG1 | THR | E | 160 | 82.235 | −33.617 | −39.469 | 1.00 | 36.87 | E |
| ATOM | 15694 | CG2 | THR | E | 160 | 83.832 | −34.851 | −40.757 | 1.00 | 36.87 | E |
| ATOM | 15695 | C | THR | E | 160 | 82.124 | −34.560 | −43.074 | 1.00 | 36.46 | E |
| ATOM | 15696 | O | THR | E | 160 | 81.390 | −35.525 | −43.249 | 1.00 | 36.46 | E |
| ATOM | 15697 | N | VAL | E | 161 | 83.098 | −34.239 | −43.926 | 1.00 | 45.76 | E |
| ATOM | 15698 | CA | VAL | E | 161 | 83.408 | −35.102 | −45.067 | 1.00 | 45.76 | E |
| ATOM | 15699 | CB | VAL | E | 161 | 83.400 | −34.364 | −46.425 | 1.00 | 61.73 | E |
| ATOM | 15700 | CG1 | VAL | E | 161 | 82.132 | −33.583 | −46.581 | 1.00 | 46.24 | E |
| ATOM | 15701 | CG2 | VAL | E | 161 | 84.587 | −33.474 | −46.544 | 1.00 | 46.24 | E |
| ATOM | 15702 | C | VAL | E | 161 | 84.807 | −35.654 | −44.799 | 1.00 | 45.76 | E |
| ATOM | 15703 | O | VAL | E | 161 | 85.699 | −34.918 | −44.374 | 1.00 | 45.76 | E |
| ATOM | 15704 | N | THR | E | 162 | 84.985 | −36.952 | −45.027 | 1.00 | 51.73 | E |
| ATOM | 15705 | CA | THR | E | 162 | 86.258 | −37.610 | −44.773 | 1.00 | 51.73 | E |
| ATOM | 15706 | CB | THR | E | 162 | 86.114 | −38.635 | −43.623 | 1.00 | 94.39 | E |
| ATOM | 15707 | OG1 | THR | E | 162 | 85.163 | −38.150 | −42.668 | 1.00 | 65.72 | E |
| ATOM | 15708 | CG2 | THR | E | 162 | 87.446 | −38.847 | −42.920 | 1.00 | 65.72 | E |
| ATOM | 15709 | C | THR | E | 162 | 86.753 | −38.348 | −46.009 | 1.00 | 51.73 | E |
| ATOM | 15710 | O | THR | E | 162 | 85.958 | −38.817 | −46.818 | 1.00 | 51.73 | E |
| ATOM | 15711 | N | LEU | E | 163 | 88.069 | −38.448 | −46.162 | 1.00 | 48.09 | E |
| ATOM | 15712 | CA | LEU | E | 163 | 88.642 | −39.172 | −47.287 | 1.00 | 48.09 | E |
| ATOM | 15713 | CB | LEU | E | 163 | 90.082 | −38.752 | −47.511 | 1.00 | 96.09 | E |
| ATOM | 15714 | CG | LEU | E | 163 | 90.333 | −38.219 | −48.919 | 1.00 | 44.18 | E |
| ATOM | 15715 | CD1 | LEU | E | 163 | 91.828 | −38.039 | −49.123 | 1.00 | 44.18 | E |
| ATOM | 15716 | CD2 | LEU | E | 163 | 89.778 | −39.170 | −49.953 | 1.00 | 44.18 | E |
| ATOM | 15717 | C | LEU | E | 163 | 88.593 | −40.679 | −47.010 | 1.00 | 48.09 | E |
| ATOM | 15718 | O | LEU | E | 163 | 88.876 | −41.512 | −47.894 | 1.00 | 48.09 | E |
| ATOM | 15719 | N | LYS | E | 164 | 88.205 | −41.030 | −45.783 | 1.00 | 200.84 | E |
| ATOM | 15720 | CA | LYS | E | 164 | 88.116 | −42.431 | −45.385 | 1.00 | 200.84 | E |
| ATOM | 15721 | CB | LYS | E | 164 | 86.901 | −43.099 | −46.039 | 1.00 | 110.06 | E |
| ATOM | 15722 | CG | LYS | E | 164 | 65.723 | −43.294 | −45.095 | 1.00 | 109.81 | E |
| ATOM | 15723 | CD | LYS | E | 164 | 86.093 | −44.218 | −43.938 | 1.00 | 109.81 | E |
| ATOM | 15724 | CE | LYS | E | 164 | 84.928 | −44.440 | −42.977 | 1.00 | 109.81 | E |
| ATOM | 15725 | NZ | LYS | E | 164 | 85.268 | −45.444 | −41.919 | 1.00 | 109.81 | E |
| ATOM | 15726 | C | LYS | E | 164 | 89.383 | −43.129 | −45.838 | 1.00 | 200.84 | E |
| ATOM | 15727 | O | LYS | E | 164 | 90.394 | −42.472 | −46.086 | 1.00 | 200.84 | E |
| ATOM | 15728 | N | ASP | E | 165 | 89.341 | −44.453 | −45.939 | 1.00 | 130.08 | E |
| ATOM | 15729 | CA | ASP | E | 165 | 90.511 | −45.187 | −46.400 | 1.00 | 130.08 | E |
| ATOM | 15730 | CB | ASP | E | 165 | 90.251 | −46.706 | −46.342 | 1.00 | 140.69 | E |
| ATOM | 15731 | CG | ASP | E | 165 | 88.927 | −47.109 | −46.982 | 1.00 | 140.69 | E |
| ATOM | 15732 | OD1 | ASP | E | 165 | 88.772 | −46.942 | −48.210 | 1.00 | 140.69 | E |
| ATOM | 15733 | OD2 | ASP | E | 165 | 88.039 | −47.600 | −46.254 | 1.00 | 140.69 | E |
| ATOM | 15734 | C | ASP | E | 165 | 90.787 | −44.718 | −47.835 | 1.00 | 130.08 | E |
| ATOM | 15735 | O | ASP | E | 165 | 90.768 | −43.524 | −48.127 | 1.00 | 130.08 | E |
| ATOM | 15736 | N | ASP | E | 166 | 91.032 | −45.642 | −48.742 | 1.00 | 70.53 | E |
| ATOM | 15737 | CA | ASP | E | 166 | 91.287 | −45.247 | −50.109 | 1.00 | 70.53 | E |
| ATOM | 15738 | CB | ASP | E | 166 | 92.343 | −46.149 | −50.695 | 1.00 | 200.84 | E |
| ATOM | 15739 | CG | ASP | E | 166 | 93.674 | −46.001 | −49.993 | 1.00 | 200.84 | E |
| ATOM | 15740 | OD1 | ASP | E | 166 | 93.756 | −46.280 | −48.778 | 1.00 | 200.84 | E |
| ATOM | 15741 | OD2 | ASP | E | 166 | 94.643 | −45.610 | −50.669 | 1.00 | 200.84 | E |
| ATOM | 15742 | C | ASP | E | 166 | 90.049 | −45.235 | −51.007 | 1.00 | 70.53 | E |
| ATOM | 15743 | O | ASP | E | 166 | 89.368 | −46.249 | −51.201 | 1.00 | 70.53 | E |
| ATOM | 15744 | N | GLY | E | 167 | 89.759 | −44.060 | −51.548 | 1.00 | 79.93 | E |
| ATOM | 15745 | CA | GLY | E | 167 | 88.620 | −43.928 | −52.428 | 1.00 | 79.93 | E |
| ATOM | 15746 | C | GLY | E | 167 | 87.735 | −42.685 | −52.335 | 1.00 | 79.93 | E |
| ATOM | 15747 | O | GLY | E | 167 | 88.159 | −41.547 | −52.554 | 1.00 | 79.93 | E |
| ATOM | 15748 | N | LEU | E | 168 | 86.472 | −42.944 | −52.019 | 1.00 | 74.08 | E |
| ATOM | 15749 | CA | LEU | E | 168 | 85.434 | −41.940 | −51.922 | 1.00 | 74.08 | E |
| ATOM | 15750 | CB | LEU | E | 168 | 84.081 | −42.653 | −51.917 | 1.00 | 87.83 | E |
| ATOM | 15751 | CG | LEU | E | 168 | 84.094 | −43.990 | −52.670 | 1.00 | 87.83 | E |
| ATOM | 15752 | CD1 | LEU | E | 168 | 82.761 | −44.717 | −52.490 | 1.00 | 87.83 | E |
| ATOM | 15753 | CD2 | LEU | E | 168 | 84.400 | −43.733 | −54.142 | 1.00 | 87.83 | E |
| ATOM | 15754 | C | LEU | E | 168 | 85.542 | −41.044 | −50.704 | 1.00 | 74.08 | E |
| ATOM | 15755 | O | LEU | E | 168 | 86.379 | −41.249 | −49.826 | 1.00 | 74.08 | E |
| ATOM | 15756 | N | TYR | E | 169 | 84.676 | −40.039 | −50.670 | 1.00 | 54.43 | E |
| ATOM | 15757 | CA | TYR | E | 169 | 84.622 | −39.104 | −49.564 | 1.00 | 54.43 | E |
| ATOM | 15758 | CB | TYR | E | 169 | 84.635 | −37.662 | −50.077 | 1.00 | 63.42 | E |
| ATOM | 15759 | CG | TYR | E | 169 | 85.941 | −37.217 | −50.669 | 1.00 | 63.42 | E |
| ATOM | 15760 | CD1 | TYR | E | 169 | 86.304 | −37.587 | −51.953 | 1.00 | 63.42 | E |
| ATOM | 15761 | CE1 | TYR | E | 169 | 87.514 | −37.176 | −52.506 | 1.00 | 63.42 | E |
| ATOM | 15762 | CD2 | TYR | E | 169 | 86.821 | −36.421 | −49.936 | 1.00 | 63.42 | E |
| ATOM | 15763 | CE2 | TYR | E | 169 | 88.037 | −36.003 | −50.474 | 1.00 | 63.42 | E |
| ATOM | 15764 | CZ | TYR | E | 169 | 88.379 | −36.384 | −51.764 | 1.00 | 63.42 | E |
| ATOM | 15765 | OH | TYR | E | 169 | 89.585 | −35.972 | −52.304 | 1.00 | 63.42 | E |
| ATOM | 15766 | C | TYR | E | 169 | 83.321 | −39.352 | −48.823 | 1.00 | 54.43 | E |
| ATOM | 15767 | O | TYR | E | 169 | 82.257 | −39.266 | −49.420 | 1.00 | 54.43 | E |
| ATOM | 15768 | N | ASP | E | 170 | 83.392 | −39.677 | −47.538 | 1.00 | 45.19 | E |
| ATOM | 15769 | CA | ASP | E | 170 | 82.169 | −39.891 | −46.778 | 1.00 | 45.19 | E |
| ATOM | 15770 | CB | ASP | E | 170 | 82.403 | −40.868 | −45.624 | 1.00 | 82.11 | E |
| ATOM | 15771 | CG | ASP | E | 170 | 82.087 | −42.292 | −46.003 | 1.00 | 82.11 | E |
| ATOM | 15772 | OD1 | ASP | E | 170 | 81.107 | −42.494 | −46.747 | 1.00 | 82.11 | E |
| ATOM | 15773 | OD2 | ASP | E | 170 | 82.800 | −43.208 | −45.551 | 1.00 | 82.11 | E |
| ATOM | 15774 | C | ASP | E | 170 | 81.659 | −38.566 | −46.229 | 1.00 | 45.19 | E |
| ATOM | 15775 | O | ASP | E | 170 | 82.426 | −37.623 | −46.046 | 1.00 | 45.19 | E |
| ATOM | 15776 | N | GLY | E | 171 | 80.359 | −38.493 | −45.982 | 1.00 | 34.37 | E |
| ATOM | 15777 | CA | GLY | E | 171 | 79.787 | −37.281 | −45.432 | 1.00 | 34.37 | E |
| ATOM | 15778 | C | GLY | E | 171 | 78.776 | −37.584 | −44.341 | 1.00 | 34.37 | E |
| ATOM | 15779 | O | GLY | E | 171 | 77.956 | −38.486 | −44.489 | 1.00 | 34.37 | E |
| ATOM | 15780 | N | VAL | E | 172 | 78.859 | −36.865 | −43.228 | 1.00 | 46.71 | E |
| ATOM | 15781 | CA | VAL | E | 172 | 77.907 | −37.027 | −42.142 | 1.00 | 46.71 | E |
| ATOM | 15782 | CB | VAL | E | 172 | 78.452 | −37.840 | −40.958 | 1.00 | 49.30 | E |
| ATOM | 15783 | CG1 | VAL | E | 172 | 78.262 | −39.291 | −41.215 | 1.00 | 41.31 | E |
| ATOM | 15784 | CG2 | VAL | E | 172 | 79.909 | −37.505 | −40.710 | 1.00 | 41.31 | E |
| ATOM | 15785 | C | VAL | E | 172 | 77.526 | −35.673 | −41.587 | 1.00 | 46.71 | E |
| ATOM | 15786 | O | VAL | E | 172 | 78.272 | −34.699 | −41.699 | 1.00 | 46.71 | E |
| ATOM | 15787 | N | PHE | E | 173 | 76.356 | −35.631 | −40.971 | 1.00 | 36.14 | E |
| ATOM | 15788 | CA | PHE | E | 173 | 75.860 | −34.425 | −40.360 | 1.00 | 36.14 | E |
| ATOM | 15789 | CB | PHE | E | 173 | 74.841 | −33.757 | −41.261 | 1.00 | 61.96 | E |
| ATOM | 15790 | CG | PHE | E | 173 | 74.079 | −32.688 | −40.574 | 1.00 | 43.78 | E |
| ATOM | 15791 | CD1 | PHE | E | 173 | 74.665 | −31.446 | −40.338 | 1.00 | 43.78 | E |
| ATOM | 15792 | CD2 | PHE | E | 173 | 72.805 | −32.948 | −40.069 | 1.00 | 43.78 | E |
| ATOM | 15793 | CE1 | PHE | E | 173 | 73.995 | −30.481 | −39.605 | 1.00 | 43.78 | E |
| ATOM | 15794 | CE2 | PHE | E | 173 | 72.122 | −31.990 | −39.328 | 1.00 | 43.78 | E |
| ATOM | 15795 | CZ | PHE | E | 173 | 72.717 | −30.754 | −39.094 | 1.00 | 43.78 | E |
| ATOM | 15796 | C | PHE | E | 173 | 75.197 | −34.796 | −39.033 | 1.00 | 36.14 | E |
| ATOM | 15797 | O | PHE | E | 173 | 74.497 | −35.808 | −38.939 | 1.00 | 36.14 | E |
| ATOM | 15798 | N | TYR | E | 174 | 75.434 | −33.998 | −37.997 | 1.00 | 39.21 | E |
| ATOM | 15799 | CA | TYR | E | 174 | 74.822 | −34.263 | −36.702 | 1.00 | 39.21 | E |
| ATOM | 15800 | CB | TYR | E | 174 | 75.847 | −34.745 | −35.676 | 1.00 | 56.79 | E |
| ATOM | 15801 | CG | TYR | E | 174 | 76.429 | −36.104 | −35.960 | 1.00 | 42.83 | E |
| ATOM | 15802 | CD1 | TYR | E | 174 | 77.726 | −36.238 | −36.456 | 1.00 | 42.83 | E |
| ATOM | 15803 | CE1 | TYR | E | 174 | 78.275 | −37.489 | −36.708 | 1.00 | 42.83 | E |
| ATOM | 15804 | CD2 | TYR | E | 174 | 75.688 | −37.266 | −35.724 | 1.00 | 42.83 | E |
| ATOM | 15805 | CE2 | TYR | E | 174 | 76.226 | −38.528 | −35.975 | 1.00 | 42.83 | E |
| ATOM | 15806 | CZ | TYR | E | 174 | 77.523 | −38.632 | −36.468 | 1.00 | 42.83 | E |
| ATOM | 15807 | OH | TYR | E | 174 | 78.084 | −39.871 | −36.726 | 1.00 | 42.83 | E |
| ATOM | 15808 | C | TYR | E | 174 | 74.189 | −32.988 | −36.186 | 1.00 | 39.21 | E |
| ATOM | 15809 | O | TYR | E | 174 | 74.762 | −31.903 | −36.302 | 1.00 | 39.21 | E |
| ATOM | 15810 | N | ASP | E | 175 | 72.995 | −33.133 | −35.627 | 1.00 | 54.47 | E |
| ATOM | 15811 | CA | ASP | E | 175 | 72.253 | −32.023 | −35.053 | 1.00 | 54.47 | E |
| ATOM | 15812 | CB | ASP | E | 175 | 70.864 | −31.929 | −35.689 | 1.00 | 66.56 | E |
| ATOM | 15813 | CG | ASP | E | 175 | 70.073 | −30.724 | −35.205 | 1.00 | 54.77 | E |
| ATOM | 15814 | OD1 | ASP | E | 175 | 70.261 | −30.309 | −34.042 | 1.00 | 54.77 | E |
| ATOM | 15815 | OD2 | ASP | E | 175 | 69.245 | −30.199 | −35.987 | 1.00 | 54.77 | E |
| ATOM | 15816 | C | ASP | E | 175 | 72.127 | −32.391 | −33.577 | 1.00 | 54.47 | E |
| ATOM | 15817 | O | ASP | E | 175 | 71.485 | −33.383 | −33.235 | 1.00 | 54.47 | E |
| ATOM | 15818 | N | HIS | E | 176 | 72.750 | −31.607 | −32.703 | 1.00 | 62.67 | E |
| ATOM | 15819 | CA | HIS | E | 176 | 72.696 | −31.876 | −31.271 | 1.00 | 62.67 | E |
| ATOM | 15820 | CB | HIS | E | 176 | 74.102 | −32.127 | −30.727 | 1.00 | 84.70 | E |
| ATOM | 15821 | CG | HIS | E | 176 | 74.663 | −33.471 | −31.068 | 1.00 | 53.83 | E |
| ATOM | 15822 | CD2 | HIS | E | 176 | 75.650 | −33.839 | −31.921 | 1.00 | 53.83 | E |
| ATOM | 15823 | ND1 | HIS | E | 176 | 74.219 | −34.633 | −30.474 | 1.00 | 53.83 | E |
| ATOM | 15824 | CE1 | HIS | E | 176 | 74.908 | −35.659 | −30.943 | 1.00 | 53.83 | E |
| ATOM | 15825 | NE2 | HIS | E | 176 | 75.783 | −35.205 | −31.822 | 1.00 | 53.83 | E |
| ATOM | 15826 | C | HIS | E | 176 | 72.091 | −30.736 | −30.476 | 1.00 | 62.67 | E |
| ATOM | 15827 | O | HIS | E | 176 | 72.537 | −29.592 | −30.586 | 1.00 | 62.67 | E |
| ATOM | 15828 | N | TYR | E | 177 | 71.065 | −31.045 | −29.690 | 1.00 | 42.70 | E |
| ATOM | 15829 | CA | TYR | E | 177 | 70.459 | −30.054 | −28.804 | 1.00 | 42.70 | E |
| ATOM | 15830 | CB | TYR | E | 177 | 68.946 | −30.167 | −28.790 | 1.00 | 49.61 | E |
| ATOM | 15831 | CG | TYR | E | 177 | 68.331 | −29.215 | −27.802 | 1.00 | 42.62 | E |
| ATOM | 15832 | CD1 | TYR | E | 177 | 68.381 | −27.840 | −28.010 | 1.00 | 42.62 | E |
| ATOM | 15833 | CE1 | TYR | E | 177 | 67.861 | −26.952 | −27.078 | 1.00 | 42.62 | E |
| ATOM | 15834 | CD2 | TYR | E | 177 | 67.741 | −29.683 | −26.631 | 1.00 | 42.62 | E |
| ATOM | 15835 | CE2 | TYR | E | 177 | 67.221 | −28.810 | −25.695 | 1.00 | 42.62 | E |
| ATOM | 15836 | CZ | TYR | E | 177 | 67.281 | −27.440 | −25.923 | 1.00 | 42.62 | E |
| ATOM | 15837 | OH | TYR | E | 177 | 66.751 | −26.566 | −25.000 | 1.00 | 42.62 | E |
| ATOM | 15838 | C | TYR | E | 177 | 71.020 | −30.483 | −27.450 | 1.00 | 42.70 | E |
| ATOM | 15839 | O | TYR | E | 177 | 70.726 | −31.583 | −26.998 | 1.00 | 42.70 | E |
| ATOM | 15840 | N | ASN | E | 178 | 71.824 | −29.638 | −26.802 | 1.00 | 44.78 | E |
| ATOM | 15841 | CA | ASN | E | 178 | 72.455 | −30.030 | −25.541 | 1.00 | 44.78 | E |
| ATOM | 15842 | CB | ASN | E | 178 | 71.432 | −30.388 | −24.460 | 1.00 | 61.03 | E |
| ATOM | 15843 | CG | ASN | E | 178 | 70.744 | −29.174 | −23.888 | 1.00 | 53.50 | E |
| ATOM | 15844 | OD1 | ASN | E | 178 | 71.158 | −28.041 | −24.138 | 1.00 | 53.50 | E |
| ATOM | 15845 | ND2 | ASN | E | 178 | 69.693 | −29.399 | −23.104 | 1.00 | 53.50 | E |
| ATOM | 15846 | C | ASN | E | 178 | 73.227 | −31.268 | −25.942 | 1.00 | 44.78 | E |
| ATOM | 15847 | O | ASN | E | 178 | 73.860 | −31.277 | −26.992 | 1.00 | 44.78 | E |
| ATOM | 15848 | N | ASP | E | 179 | 73.171 | −32.316 | −25.131 | 1.00 | 48.57 | E |
| ATOM | 15849 | CA | ASP | E | 179 | 73.878 | −33.546 | −25.472 | 1.00 | 48.57 | E |
| ATOM | 15850 | CB | ASP | E | 179 | 74.527 | −34.173 | −24.227 | 1.00 | 42.85 | E |
| ATOM | 15851 | CG | ASP | E | 179 | 73.540 | −34.433 | −23.104 | 1.00 | 46.80 | E |
| ATOM | 15852 | OD1 | ASP | E | 179 | 72.337 | −34.151 | −23.283 | 1.00 | 46.80 | E |
| ATOM | 15853 | OD2 | ASP | E | 179 | 73.973 | −34.921 | −22.033 | 1.00 | 46.80 | E |
| ATOM | 15854 | C | ASP | E | 179 | 72.952 | −34.557 | −26.148 | 1.00 | 48.57 | E |
| ATOM | 15855 | O | ASP | E | 179 | 73.295 | −35.720 | −26.278 | 1.00 | 48.57 | E |
| ATOM | 15856 | N | GLN | E | 180 | 71.787 | −34.096 | −26.593 | 1.00 | 37.71 | E |
| ATOM | 15857 | CA | GLN | E | 180 | 70.810 | −34.947 | −27.258 | 1.00 | 37.71 | E |
| ATOM | 15858 | CB | GLN | E | 180 | 69.400 | −34.500 | −26.886 | 1.00 | 58.82 | E |
| ATOM | 15859 | CG | GLN | E | 180 | 69.125 | −34.434 | −25.416 | 1.00 | 50.45 | E |
| ATOM | 15860 | CD | GLN | E | 180 | 69.337 | −35.759 | −24.742 | 1.00 | 50.45 | E |
| ATOM | 15861 | OE1 | GLN | E | 180 | 70.362 | −35.980 | −24.119 | 1.00 | 50.45 | E |
| ATOM | 15862 | NE2 | GLN | E | 180 | 68.371 | −36.656 | −24.873 | 1.00 | 50.45 | E |
| ATOM | 15863 | C | GLN | E | 180 | 70.901 | −34.959 | −28.795 | 1.00 | 37.71 | E |
| ATOM | 15864 | O | GLN | E | 180 | 70.730 | −33.928 | −29.454 | 1.00 | 37.71 | E |
| ATOM | 15865 | N | LEU | E | 181 | 71.149 | −36.127 | −29.372 | 1.00 | 34.70 | E |
| ATOM | 15866 | CA | LEU | E | 181 | 71.190 | −36.227 | −30.826 | 1.00 | 34.70 | E |
| ATOM | 15867 | CB | LEU | E | 181 | 71.676 | −37.610 | −31.255 | 1.00 | 45.14 | E |
| ATOM | 15868 | CG | LEU | E | 181 | 71.624 | −37.843 | −32.762 | 1.00 | 32.57 | E |
| ATOM | 15869 | CD1 | LEU | E | 181 | 72.530 | −36.867 | −33.474 | 1.00 | 32.57 | E |
| ATOM | 15870 | CD2 | LEU | E | 181 | 72.032 | −39.275 | −33.071 | 1.00 | 32.57 | E |
| ATOM | 15871 | C | LEU | E | 181 | 69.773 | −35.978 | −31.359 | 1.00 | 34.70 | E |
| ATOM | 15872 | O | LEU | E | 181 | 68.805 | −36.561 | −30.866 | 1.00 | 34.70 | E |
| ATOM | 15873 | N | VAL | E | 182 | 69.656 | −35.103 | −32.353 | 1.00 | 40.56 | E |
| ATOM | 15874 | CA | VAL | E | 182 | 68.356 | −34.785 | −32.923 | 1.00 | 40.56 | E |
| ATOM | 15875 | CB | VAL | E | 182 | 68.119 | −33.276 | −32.960 | 1.00 | 56.47 | E |
| ATOM | 15876 | CG1 | VAL | E | 182 | 66.784 | −32.972 | −33.606 | 1.00 | 34.95 | E |
| ATOM | 15877 | CG2 | VAL | E | 182 | 68.158 | −32.726 | −31.545 | 1.00 | 34.95 | E |
| ATOM | 15878 | C | VAL | E | 182 | 68.231 | −35.317 | −34.326 | 1.00 | 40.56 | E |
| ATOM | 15879 | O | VAL | E | 182 | 67.180 | −35.822 | −34.715 | 1.00 | 40.56 | E |
| ATOM | 15880 | N | THR | E | 183 | 69.314 | −35.215 | −35.084 | 1.00 | 51.13 | E |
| ATOM | 15881 | CA | THR | E | 183 | 69.320 | −35.683 | −36.457 | 1.00 | 51.13 | E |
| ATOM | 15882 | CB | THR | E | 183 | 68.909 | −34.562 | −37.412 | 1.00 | 58.44 | E |
| ATOM | 15883 | OG1 | THR | E | 183 | 67.556 | −34.188 | −37.152 | 1.00 | 45.03 | E |
| ATOM | 15884 | CG2 | THR | E | 183 | 69.063 | −35.009 | −38.858 | 1.00 | 45.03 | E |
| ATOM | 15885 | C | THR | E | 183 | 70.681 | −36.168 | −36.915 | 1.00 | 51.13 | E |
| ATOM | 15886 | O | THR | E | 183 | 71.711 | −35.586 | −36.576 | 1.00 | 51.13 | E |
| ATOM | 15887 | N | LYS | E | 184 | 70.688 | −37.248 | −37.681 | 1.00 | 45.30 | E |
| ATOM | 15888 | CA | LYS | E | 184 | 71.926 | −37.745 | −38.254 | 1.00 | 45.30 | E |
| ATOM | 15889 | CB | LYS | E | 184 | 72.352 | −39.077 | −37.667 | 1.00 | 49.40 | E |
| ATOM | 15890 | CG | LYS | E | 184 | 73.614 | −39.577 | −38.331 | 1.00 | 41.24 | E |
| ATOM | 15891 | CD | LYS | E | 184 | 74.044 | −40.935 | −37.835 | 1.00 | 41.24 | E |
| ATOM | 15892 | CE | LYS | E | 184 | 75.281 | −41.420 | −38.575 | 1.00 | 41.24 | E |
| ATOM | 15893 | NZ | LYS | E | 184 | 75.634 | −42.817 | −38.193 | 1.00 | 41.24 | E |
| ATOM | 15894 | C | LYS | E | 184 | 71.655 | −37.930 | −39.735 | 1.00 | 45.30 | E |
| ATOM | 15895 | O | LYS | E | 184 | 70.620 | −38.459 | −40.127 | 1.00 | 45.30 | E |
| ATOM | 15896 | N | ILE | E | 185 | 72.576 | −37.470 | −40.559 | 1.00 | 45.04 | E |
| ATOM | 15897 | CA | ILE | E | 185 | 72.429 | −37.601 | −41.994 | 1.00 | 45.04 | E |
| ATOM | 15898 | CB | ILE | E | 185 | 72.028 | −36.253 | −42.634 | 1.00 | 51.71 | E |
| ATOM | 15899 | CG2 | ILE | E | 185 | 72.191 | −36.312 | −44.128 | 1.00 | 20.63 | E |
| ATOM | 15900 | CG1 | ILE | E | 185 | 70.574 | −35.928 | −42.305 | 1.00 | 20.63 | E |
| ATOM | 15901 | CD1 | ILE | E | 185 | 70.156 | −34.517 | −42.704 | 1.00 | 20.63 | E |
| ATOM | 15902 | C | ILE | E | 185 | 73.765 | −38.050 | −42.558 | 1.00 | 45.04 | E |
| ATOM | 15903 | O | ILE | E | 185 | 74.825 | −37.596 | −42.114 | 1.00 | 45.04 | E |
| ATOM | 15904 | N | SER | E | 186 | 73.724 | −38.964 | −43.517 | 1.00 | 45.81 | E |
| ATOM | 15905 | CA | SER | E | 186 | 74.958 | −39.422 | −44.138 | 1.00 | 45.81 | E |
| ATOM | 15906 | CB | SER | E | 186 | 75.396 | −40.763 | −43.545 | 1.00 | 79.56 | E |
| ATOM | 15907 | OG | SER | E | 186 | 74.299 | −41.637 | −43.419 | 1.00 | 68.67 | E |
| ATOM | 15908 | C | SER | E | 186 | 74.802 | −39.494 | −45.653 | 1.00 | 45.81 | E |
| ATOM | 15909 | O | SER | E | 186 | 73.702 | −39.650 | −46.172 | 1.00 | 45.81 | E |
| ATOM | 15910 | N | TYR | E | 187 | 75.911 | −39.333 | −46.357 | 1.00 | 52.04 | E |
| ATOM | 15911 | CA | TYR | E | 187 | 75.908 | −39.353 | −47.804 | 1.00 | 52.04 | E |
| ATOM | 15912 | CB | TYR | E | 187 | 77.178 | −38.712 | −48.325 | 1.00 | 90.60 | E |
| ATOM | 15913 | CG | TYR | E | 187 | 77.186 | −38.590 | −49.811 | 1.00 | 44.81 | E |
| ATOM | 15914 | CD1 | TYR | E | 187 | 76.368 | −37.659 | −50.451 | 1.00 | 44.81 | E |
| ATOM | 15915 | CE1 | TYR | E | 187 | 76.383 | −37.516 | −51.823 | 1.00 | 44.81 | E |
| ATOM | 15916 | CD2 | TYR | E | 187 | 78.021 | −39.395 | −50.588 | 1.00 | 44.81 | E |
| ATOM | 15917 | CE2 | TYR | E | 187 | 78.044 | −39.264 | −51.975 | 1.00 | 44.81 | E |
| ATOM | 15918 | CZ | TYR | E | 187 | 77.222 | −38.314 | −52.583 | 1.00 | 44.81 | E |
| ATOM | 15919 | OH | TYR | E | 187 | 77.270 | −38.138 | −53.946 | 1.00 | 44.81 | E |
| ATOM | 15920 | C | TYR | E | 187 | 75.822 | −40.769 | −48.334 | 1.00 | 52.04 | E |
| ATOM | 15921 | O | TYR | E | 187 | 76.688 | −41.601 | −48.049 | 1.00 | 52.04 | E |
| ATOM | 15922 | N | ASN | E | 188 | 74.774 | −41.035 | −49.107 | 1.00 | 54.72 | E |
| ATOM | 15923 | CA | ASN | E | 188 | 74.544 | −42.344 | −49.711 | 1.00 | 54.72 | E |
| ATOM | 15924 | CB | ASN | E | 188 | 73.053 | −42.512 | −49.969 | 1.00 | 186.58 | E |
| ATOM | 15925 | CG | ASN | E | 188 | 72.713 | −43.848 | −50.563 | 1.00 | 47.61 | E |
| ATOM | 15926 | OD1 | ASN | E | 188 | 73.269 | −44.256 | −51.578 | 1.00 | 47.61 | E |
| ATOM | 15927 | ND2 | ASN | E | 188 | 71.788 | −44.543 | −49.936 | 1.00 | 47.61 | E |
| ATOM | 15928 | C | ASN | E | 188 | 75.319 | −42.427 | −51.036 | 1.00 | 54.72 | E |
| ATOM | 15929 | O | ASN | E | 188 | 74.996 | −41.710 | −51.987 | 1.00 | 54.72 | E |
| ATOM | 15930 | N | HIS | E | 189 | 76.328 | −43.300 | −51.105 | 1.00 | 40.83 | E |
| ATOM | 15931 | CA | HIS | E | 189 | 77.134 | −43.416 | −52.316 | 1.00 | 40.83 | E |
| ATOM | 15932 | CB | HIS | E | 189 | 78.397 | −44.198 | −52.006 | 1.00 | 106.43 | E |
| ATOM | 15933 | CG | HIS | E | 189 | 79.370 | −43.416 | −51.195 | 1.00 | 66.97 | E |
| ATOM | 15934 | CD2 | HIS | E | 189 | 79.621 | −43.409 | −49.866 | 1.00 | 66.97 | E |
| ATOM | 15935 | ND1 | HIS | E | 189 | 80.156 | −42.425 | −51.743 | 1.00 | 66.97 | E |
| ATOM | 15936 | CE1 | HIS | E | 189 | 80.851 | −41.839 | −50.786 | 1.00 | 66.97 | E |
| ATOM | 15937 | NE2 | HIS | E | 189 | 80.544 | −42.418 | −49.637 | 1.00 | 66.97 | E |
| ATOM | 15938 | C | HIS | E | 189 | 76.416 | −44.003 | −53.516 | 1.00 | 40.83 | E |
| ATOM | 15939 | O | HIS | E | 189 | 76.749 | −43.693 | −54.665 | 1.00 | 40.83 | E |
| ATOM | 15940 | N | GLU | E | 190 | 75.421 | −44.841 | −53.245 | 1.00 | 49.11 | E |
| ATOM | 15941 | CA | GLU | E | 190 | 74.644 | −45.479 | −54.293 | 1.00 | 49.11 | E |
| ATOM | 15942 | CB | GLU | E | 190 | 73.784 | −46.577 | −53.676 | 1.00 | 104.31 | E |
| ATOM | 15943 | CG | GLU | E | 190 | 73.390 | −47.679 | −54.614 | 1.00 | 78.73 | E |
| ATOM | 15944 | CD | GLU | E | 190 | 72.651 | −48.796 | −53.899 | 1.00 | 78.73 | E |
| ATOM | 15945 | OE1 | GLU | E | 190 | 71.539 | −48.536 | −53.387 | 1.00 | 78.73 | E |
| ATOM | 15946 | OE2 | GLU | E | 190 | 73.181 | −49.930 | −53.841 | 1.00 | 78.73 | E |
| ATOM | 15947 | C | GLU | E | 190 | 73.770 | −44.403 | −54.940 | 1.00 | 49.11 | E |
| ATOM | 15948 | O | GLU | E | 190 | 73.840 | −44.148 | −56.145 | 1.00 | 49.11 | E |
| ATOM | 15949 | N | THR | E | 191 | 72.972 | −43.742 | −54.112 | 1.00 | 47.50 | E |
| ATOM | 15950 | CA | THR | E | 191 | 72.053 | −42.691 | −54.550 | 1.00 | 47.50 | E |
| ATOM | 15951 | CB | THR | E | 191 | 71.006 | −42.443 | −53.486 | 1.00 | 65.12 | E |
| ATOM | 15952 | OG1 | THR | E | 191 | 70.235 | −43.624 | −53.305 | 1.00 | 65.12 | E |
| ATOM | 15953 | CG2 | THR | E | 191 | 70.093 | −41.351 | −53.897 | 1.00 | 65.12 | E |
| ATOM | 15954 | C | THR | E | 191 | 72.688 | −41.340 | −54.883 | 1.00 | 47.50 | E |
| ATOM | 15955 | O | THR | E | 191 | 72.156 | −40.579 | −55.686 | 1.00 | 47.50 | E |
| ATOM | 15956 | N | ARG | E | 192 | 73.809 | −41.027 | −54.243 | 1.00 | 61.52 | E |
| ATOM | 15957 | CA | ARG | E | 192 | 74.510 | −39.776 | −54.483 | 1.00 | 61.52 | E |
| ATOM | 15958 | CB | ARG | E | 192 | 74.695 | −39.535 | −55.983 | 1.00 | 51.52 | E |
| ATOM | 15959 | CG | ARG | E | 192 | 75.617 | −40.545 | −56.642 | 1.00 | 51.52 | E |
| ATOM | 15960 | CD | ARG | E | 192 | 76.985 | −40.540 | −56.005 | 1.00 | 51.52 | E |
| ATOM | 15961 | NE | ARG | E | 192 | 77.815 | −41.631 | −56.496 | 1.00 | 51.52 | E |
| ATOM | 15962 | CZ | ARG | E | 192 | 78.264 | −41.735 | −57.748 | 1.00 | 51.52 | E |
| ATOM | 15963 | NH1 | ARG | E | 192 | 77.966 | −40.804 | −58.639 | 1.00 | 51.52 | E |
| ATOM | 15964 | NH2 | ARG | E | 192 | 79.006 | −42.777 | −58.118 | 1.00 | 51.52 | E |
| ATOM | 15965 | C | ARG | E | 192 | 73.866 | −38.558 | −53.841 | 1.00 | 61.52 | E |
| ATOM | 15966 | O | ARG | E | 192 | 73.965 | −37.451 | −54.358 | 1.00 | 61.52 | E |
| ATOM | 15967 | N | HIS | E | 193 | 73.202 | −38.765 | −52.712 | 1.00 | 43.86 | E |
| ATOM | 15968 | CA | HIS | E | 193 | 72.601 | −37.665 | −51.958 | 1.00 | 43.86 | E |
| ATOM | 15969 | CB | HIS | E | 193 | 71.237 | −37.273 | −52.539 | 1.00 | 64.12 | E |
| ATOM | 15970 | CG | HIS | E | 193 | 70.138 | −38.254 | −52.270 | 1.00 | 64.12 | E |
| ATOM | 15971 | CD2 | HIS | E | 193 | 70.036 | −39.272 | −51.383 | 1.00 | 64.12 | E |
| ATOM | 15972 | ND1 | HIS | E | 193 | 68.946 | −38.227 | −52.954 | 1.00 | 64.12 | E |
| ATOM | 15973 | CE1 | HIS | E | 193 | 68.158 | −39.186 | −52.505 | 1.00 | 64.12 | E |
| ATOM | 15974 | NE2 | HIS | E | 193 | 68.796 | −39.836 | −51.551 | 1.00 | 64.12 | E |
| ATOM | 15975 | C | HIS | E | 193 | 72.494 | −38.116 | −50.501 | 1.00 | 43.86 | E |
| ATOM | 15976 | O | HIS | E | 193 | 72.720 | −39.294 | −50.204 | 1.00 | 43.86 | E |
| ATOM | 15977 | N | GLY | E | 194 | 72.152 | −37.210 | −49.591 | 1.00 | 49.75 | E |
| ATOM | 15978 | CA | GLY | E | 194 | 72.073 | −37.589 | −48.186 | 1.00 | 49.75 | E |
| ATOM | 15979 | C | GLY | E | 194 | 70.812 | −38.321 | −47.762 | 1.00 | 49.75 | E |
| ATOM | 15980 | O | GLY | E | 194 | 69.803 | −38.277 | −48.457 | 1.00 | 49.75 | E |
| ATOM | 15981 | N | ASN | E | 195 | 70.871 | −39.011 | −46.627 | 1.00 | 48.97 | E |
| ATOM | 15982 | CA | ASN | E | 195 | 69.716 | −39.734 | −46.093 | 1.00 | 48.97 | E |
| ATOM | 15983 | CB | ASN | E | 195 | 69.824 | −41.253 | −46.308 | 1.00 | 56.87 | E |
| ATOM | 15984 | CG | ASN | E | 195 | 69.690 | −41.661 | −47.756 | 1.00 | 56.87 | E |
| ATOM | 15985 | OD1 | ASN | E | 195 | 68.804 | −41.193 | −48.471 | 1.00 | 56.87 | E |
| ATOM | 15986 | ND2 | ASN | E | 195 | 70.564 | −42.560 | −48.194 | 1.00 | 56.87 | E |
| ATOM | 15987 | C | ASN | E | 195 | 69.661 | −39.489 | −44.595 | 1.00 | 48.97 | E |
| ATOM | 15988 | O | ASN | E | 195 | 70.700 | −39.439 | −43.923 | 1.00 | 48.97 | E |
| ATOM | 15989 | N | VAL | E | 196 | 68.457 | −39.333 | −44.063 | 1.00 | 41.21 | E |
| ATOM | 15990 | CA | VAL | E | 196 | 68.325 | −39.137 | −42.634 | 1.00 | 41.21 | E |
| ATOM | 15991 | CB | VAL | E | 196 | 66.991 | −38.497 | −42.254 | 1.00 | 35.00 | E |
| ATOM | 15992 | CG1 | VAL | E | 196 | 66.896 | −38.402 | −40.742 | 1.00 | 35.00 | E |
| ATOM | 15993 | CG2 | VAL | E | 196 | 66.872 | −37.114 | −42.883 | 1.00 | 35.00 | E |
| ATOM | 15994 | C | VAL | E | 196 | 68.380 | −40.515 | −42.001 | 1.00 | 41.21 | E |
| ATOM | 15995 | O | VAL | E | 196 | 67.530 | −41.357 | −42.276 | 1.00 | 41.21 | E |
| ATOM | 15996 | N | ASN | E | 197 | 69.385 | −40.754 | −41.163 | 1.00 | 60.34 | E |
| ATOM | 15997 | CA | ASN | E | 197 | 69.535 | −42.050 | −40.518 | 1.00 | 60.34 | E |
| ATOM | 15998 | CB | ASN | E | 197 | 71.009 | −42.392 | −40.308 | 1.00 | 66.72 | E |
| ATOM | 15999 | CG | ASN | E | 197 | 71.839 | −42.180 | −41.548 | 1.00 | 66.72 | E |
| ATOM | 16000 | OD1 | ASN | E | 197 | 71.467 | −42.607 | −42.643 | 1.00 | 66.72 | E |
| ATOM | 16001 | ND2 | ASN | E | 197 | 72.985 | −41.518 | −41.383 | 1.00 | 66.72 | E |
| ATOM | 16002 | C | ASN | E | 197 | 68.854 | −42.042 | −39.171 | 1.00 | 60.34 | E |
| ATOM | 16003 | O | ASN | E | 197 | 68.350 | −43.059 | −38.715 | 1.00 | 60.34 | E |
| ATOM | 16004 | N | PHE | E | 198 | 68.835 | −40.885 | −38.531 | 1.00 | 48.25 | E |
| ATOM | 16005 | CA | PHE | E | 198 | 68.236 | −40.785 | −37.221 | 1.00 | 48.25 | E |
| ATOM | 16006 | CB | PHE | E | 198 | 69.324 | −40.867 | −36.162 | 1.00 | 40.87 | E |
| ATOM | 16007 | CG | PHE | E | 198 | 68.803 | −40.790 | −34.760 | 1.00 | 40.87 | E |
| ATOM | 16008 | CD1 | PHE | E | 198 | 68.473 | −41.953 | −34.065 | 1.00 | 40.87 | E |
| ATOM | 16009 | CD2 | PHE | E | 198 | 68.602 | −39.551 | −34.140 | 1.00 | 40.87 | E |
| ATOM | 16010 | CE1 | PHE | E | 198 | 67.954 | −41.892 | −32.787 | 1.00 | 40.87 | E |
| ATOM | 16011 | CE2 | PHE | E | 198 | 68.080 | −39.481 | −32.858 | 1.00 | 40.87 | E |
| ATOM | 16012 | CZ | PHE | E | 198 | 67.757 | −40.653 | −32.183 | 1.00 | 40.87 | E |
| ATOM | 16013 | C | PHE | E | 198 | 67.467 | −39.495 | −37.030 | 1.00 | 48.25 | E |
| ATOM | 16014 | O | PHE | E | 198 | 67.830 | −38.451 | −37.558 | 1.00 | 48.25 | E |
| ATOM | 16015 | N | ARG | E | 199 | 66.399 | −39.572 | −36.257 | 1.00 | 46.08 | E |
| ATOM | 16016 | CA | ARG | E | 199 | 65.598 | −38.404 | −35.964 | 1.00 | 46.08 | E |
| ATOM | 16017 | CB | ARG | E | 199 | 64.492 | −38.215 | −36.989 | 1.00 | 55.25 | E |
| ATOM | 16018 | CG | ARG | E | 199 | 63.625 | −37.034 | −36.653 | 1.00 | 55.25 | E |
| ATOM | 16019 | CD | ARG | E | 199 | 64.347 | −35.751 | −36.969 | 1.00 | 55.25 | E |
| ATOM | 16020 | NE | ARG | E | 199 | 63.915 | −35.246 | −38.262 | 1.00 | 55.25 | E |
| ATOM | 16021 | CZ | ARG | E | 199 | 64.704 | −34.627 | −39.129 | 1.00 | 55.25 | E |
| ATOM | 16022 | NH1 | ARG | E | 199 | 65.978 | −34.437 | −38.843 | 1.00 | 55.25 | E |
| ATOM | 16023 | NH2 | ARG | E | 199 | 64.214 | −34.186 | −40.283 | 1.00 | 55.25 | E |
| ATOM | 16024 | C | ARG | E | 199 | 64.977 | −38.622 | −34.601 | 1.00 | 46.08 | E |
| ATOM | 16025 | O | ARG | E | 199 | 64.221 | −39.568 | −34.403 | 1.00 | 46.08 | E |
| ATOM | 16026 | N | ALA | E | 200 | 65.307 | −37.757 | −33.654 | 1.00 | 51.41 | E |
| ATOM | 16027 | CA | ALA | E | 200 | 64.754 | −37.877 | −32.318 | 1.00 | 51.41 | E |
| ATOM | 16028 | CB | ALA | E | 200 | 65.258 | −36.742 | −31.443 | 1.00 | 38.61 | E |
| ATOM | 16029 | C | ALA | E | 200 | 63.226 | −37.858 | −32.388 | 1.00 | 51.41 | E |
| ATOM | 16030 | O | ALA | E | 200 | 62.629 | −37.066 | −33.127 | 1.00 | 51.41 | E |
| ATOM | 16031 | N | ASP | E | 201 | 62.606 | −38.741 | −31.612 | 1.00 | 78.93 | E |
| ATOM | 16032 | CA | ASP | E | 201 | 61.155 | −38.864 | −31.558 | 1.00 | 78.93 | E |
| ATOM | 16033 | CB | ASP | E | 201 | 60.783 | −40.024 | −30.633 | 1.00 | 147.71 | E |
| ATOM | 16034 | CG | ASP | E | 201 | 61.498 | −39.948 | −29.298 | 1.00 | 147.71 | E |
| ATOM | 16035 | OD1 | ASP | E | 201 | 62.738 | −40.115 | −29.282 | 1.00 | 147.71 | E |
| ATOM | 16036 | OD2 | ASP | E | 201 | 60.827 | −39.711 | −28.268 | 1.00 | 147.71 | E |
| ATOM | 16037 | C | ASP | E | 201 | 60.429 | −37.594 | −31.095 | 1.00 | 78.93 | E |
| ATOM | 16038 | O | ASP | E | 201 | 59.292 | −37.347 | −31.492 | 1.00 | 78.93 | E |
| ATOM | 16039 | N | TRP | E | 202 | 61.085 | −36.789 | −30.265 | 1.00 | 54.92 | E |
| ATOM | 16040 | CA | TRP | E | 202 | 60.464 | −35.574 | −29.741 | 1.00 | 54.92 | E |
| ATOM | 16041 | CB | TRP | E | 202 | 61.036 | −35.226 | −28.370 | 1.00 | 52.12 | E |
| ATOM | 16042 | CG | TRP | E | 202 | 62.529 | −35.126 | −28.315 | 1.00 | 52.12 | E |
| ATOM | 16043 | CD2 | TRP | E | 202 | 63.319 | −33.959 | −28.559 | 1.00 | 52.12 | E |
| ATOM | 16044 | CE2 | TRP | E | 202 | 64.673 | −34.313 | −28.335 | 1.00 | 52.12 | E |
| ATOM | 16045 | CE3 | TRP | E | 202 | 63.017 | −32.651 | −28.944 | 1.00 | 52.12 | E |
| ATOM | 16046 | CD1 | TRP | E | 202 | 63.407 | −36.116 | −27.971 | 1.00 | 52.12 | E |
| ATOM | 16047 | NE1 | TRP | E | 202 | 64.697 | −35.634 | −27.975 | 1.00 | 52.12 | E |
| ATOM | 16048 | CZ2 | TRP | E | 202 | 65.720 | −33.405 | −28.483 | 1.00 | 52.12 | E |
| ATOM | 16049 | CZ3 | TRP | E | 202 | 64.058 | −31.748 | −29.091 | 1.00 | 52.12 | E |
| ATOM | 16050 | CH2 | TRP | E | 202 | 65.396 | −32.132 | −28.860 | 1.00 | 52.12 | E |
| ATOM | 16051 | C | TRP | E | 202 | 60.561 | −34.332 | −30.603 | 1.00 | 54.92 | E |
| ATOM | 16052 | O | TRP | E | 202 | 59.940 | −33.311 | −30.292 | 1.00 | 54.92 | E |
| ATOM | 16053 | N | PHE | E | 203 | 61.325 | −34.416 | −31.686 | 1.00 | 49.16 | E |
| ATOM | 16054 | CA | PHE | E | 203 | 61.523 | −33.272 | −32.562 | 1.00 | 49.16 | E |
| ATOM | 16055 | CB | PHE | E | 203 | 63.006 | −33.187 | −32.944 | 1.00 | 57.04 | E |
| ATOM | 16056 | CG | PHE | E | 203 | 63.374 | −31.935 | −33.687 | 1.00 | 57.04 | E |
| ATOM | 16057 | CD1 | PHE | E | 203 | 63.367 | −30.701 | −33.044 | 1.00 | 57.04 | E |
| ATOM | 16058 | CD2 | PHE | E | 203 | 63.741 | −31.988 | −35.030 | 1.00 | 57.04 | E |
| ATOM | 16059 | CE1 | PHE | E | 203 | 63.722 | −29.539 | −33.725 | 1.00 | 57.04 | E |
| ATOM | 16060 | CE2 | PHE | E | 203 | 64.097 | −30.826 | −35.720 | 1.00 | 57.04 | E |
| ATOM | 16061 | CZ | PHE | E | 203 | 64.090 | −29.603 | −35.068 | 1.00 | 57.04 | E |
| ATOM | 16062 | C | PHE | E | 203 | 60.676 | −33.309 | −33.824 | 1.00 | 49.16 | E |
| ATOM | 16063 | O | PHE | E | 203 | 60.959 | −34.088 | −34.735 | 1.00 | 49.16 | E |
| ATOM | 16064 | N | ASN | E | 204 | 59.637 | −32.476 | −33.873 | 1.00 | 50.70 | E |
| ATOM | 16065 | CA | ASN | E | 204 | 58.791 | −32.387 | −35.062 | 1.00 | 50.70 | E |
| ATOM | 16066 | CB | ASN | E | 204 | 57.332 | −32.057 | −34.695 | 1.00 | 88.93 | E |
| ATOM | 16067 | CG | ASN | E | 204 | 56.483 | −31.691 | −35.916 | 1.00 | 88.93 | E |
| ATOM | 16068 | OD1 | ASN | E | 204 | 56.713 | −30.660 | −36.541 | 1.00 | 88.93 | E |
| ATOM | 16069 | ND2 | ASN | E | 204 | 55.540 | −32.552 | −36.286 | 1.00 | 88.93 | E |
| ATOM | 16070 | C | ASN | E | 204 | 59.416 | −31.251 | −35.862 | 1.00 | 50.70 | E |
| ATOM | 16071 | O | ASN | E | 204 | 59.203 | −30.080 | −35.556 | 1.00 | 50.70 | E |
| ATOM | 16072 | N | ILE | E | 205 | 60.205 | −31.591 | −36.875 | 1.00 | 53.96 | E |
| ATOM | 16073 | CA | ILE | E | 205 | 60.879 | −30.576 | −37.678 | 1.00 | 53.96 | E |
| ATOM | 16074 | CB | ILE | E | 205 | 61.740 | −31.230 | −38.785 | 1.00 | 43.86 | E |
| ATOM | 16075 | CG2 | ILE | E | 205 | 60.869 | −31.583 | −39.975 | 1.00 | 43.86 | E |
| ATOM | 16076 | CG1 | ILE | E | 205 | 62.850 | −30.262 | −39.212 | 1.00 | 43.86 | E |
| ATOM | 16077 | CD1 | ILE | E | 205 | 64.011 | −30.933 | −39.900 | 1.00 | 43.86 | E |
| ATOM | 16078 | C | ILE | E | 205 | 59.947 | −29.534 | −38.311 | 1.00 | 53.96 | E |
| ATOM | 16079 | O | ILE | E | 205 | 60.395 | −28.471 | −38.743 | 1.00 | 53.96 | E |
| ATOM | 16080 | N | SER | E | 206 | 58.656 | −29.835 | −38.367 | 1.00 | 60.50 | E |
| ATOM | 16081 | CA | SER | E | 206 | 57.700 | −28.895 | −38.938 | 1.00 | 60.50 | E |
| ATOM | 16082 | CB | SER | E | 206 | 56.389 | −29.598 | −39.277 | 1.00 | 68.77 | E |
| ATOM | 16083 | OG | SER | E | 206 | 56.593 | −30.546 | −40.305 | 1.00 | 68.77 | E |
| ATOM | 16084 | C | SER | E | 206 | 57.429 | −27.763 | −37.965 | 1.00 | 60.50 | E |
| ATOM | 16085 | O | SER | E | 206 | 56.983 | −26.688 | −38.356 | 1.00 | 60.50 | E |
| ATOM | 16086 | N | ARG | E | 207 | 57.697 | −28.014 | −36.692 | 1.00 | 61.48 | E |
| ATOM | 16087 | CA | ARG | E | 207 | 57.488 | −27.006 | −35.678 | 1.00 | 61.48 | E |
| ATOM | 16088 | CB | ARG | E | 207 | 57.030 | −27.657 | −34.375 | 1.00 | 86.75 | E |
| ATOM | 16089 | CG | ARG | E | 207 | 55.592 | −28.133 | −34.411 | 1.00 | 86.75 | E |
| ATOM | 16090 | CD | ARG | E | 207 | 54.950 | −28.043 | −33.040 | 1.00 | 86.75 | E |
| ATOM | 16091 | NE | ARG | E | 207 | 55.370 | −29.122 | −32.156 | 1.00 | 86.75 | E |
| ATOM | 16092 | CZ | ARG | E | 207 | 55.382 | −29.032 | −30.827 | 1.00 | 86.75 | E |
| ATOM | 16093 | NH1 | ARG | E | 207 | 55.001 | −27.903 | −30.233 | 1.00 | 86.75 | E |
| ATOM | 16094 | NH2 | ARG | E | 207 | 55.762 | −30.073 | −30.090 | 1.00 | 86.75 | E |
| ATOM | 16095 | C | ARG | E | 207 | 58.750 | −26.183 | −35.436 | 1.00 | 61.48 | E |
| ATOM | 16096 | O | ARG | E | 207 | 58.815 | −25.420 | −34.472 | 1.00 | 61.48 | E |
| ATOM | 16097 | N | SER | E | 208 | 59.750 | −26.336 | −36.305 | 1.00 | 52.12 | E |
| ATOM | 16098 | CA | SER | E | 208 | 60.993 | −25.586 | −36.164 | 1.00 | 52.12 | E |
| ATOM | 16099 | CB | SER | E | 208 | 62.137 | −26.494 | −35.748 | 1.00 | 56.56 | E |
| ATOM | 16100 | OG | SER | E | 208 | 63.296 | −25.715 | −35.518 | 1.00 | 56.56 | E |
| ATOM | 16101 | C | SER | E | 208 | 61.351 | −24.897 | −37.463 | 1.00 | 52.12 | E |
| ATOM | 16102 | O | SER | E | 208 | 62.317 | −25.264 | −38.135 | 1.00 | 52.12 | E |
| ATOM | 16103 | N | PRO | E | 209 | 60.571 | −23.874 | −37.826 | 1.00 | 51.81 | E |
| ATOM | 16104 | CD | PRO | E | 209 | 59.508 | −23.354 | −36.951 | 1.00 | 50.90 | E |
| ATOM | 16105 | CA | PRO | E | 209 | 60.694 | −23.044 | −39.029 | 1.00 | 51.81 | E |
| ATOM | 16106 | CB | PRO | E | 209 | 59.801 | −21.848 | −38.712 | 1.00 | 50.90 | E |
| ATOM | 16107 | CG | PRO | E | 209 | 58.739 | −22.455 | −37.882 | 1.00 | 50.90 | E |
| ATOM | 16108 | C | PRO | E | 209 | 62.108 | −22.592 | −39.349 | 1.00 | 51.81 | E |
| ATOM | 16109 | O | PRO | E | 209 | 62.448 | −22.391 | −40.515 | 1.00 | 51.81 | E |
| ATOM | 16110 | N | HIS | E | 210 | 62.927 | −22.434 | −38.311 | 1.00 | 52.94 | E |
| ATOM | 16111 | CA | HIS | E | 210 | 64.279 | −21.950 | −38.504 | 1.00 | 52.94 | E |
| ATOM | 16112 | CB | HIS | E | 210 | 64.585 | −20.915 | −37.437 | 1.00 | 56.19 | E |
| ATOM | 16113 | CG | HIS | E | 210 | 63.699 | −19.717 | −37.524 | 1.00 | 56.19 | E |
| ATOM | 16114 | CD2 | HIS | E | 210 | 62.665 | −19.318 | −36.750 | 1.00 | 56.19 | E |
| ATOM | 16115 | ND1 | HIS | E | 210 | 63.792 | −18.798 | −38.546 | 1.00 | 56.19 | E |
| ATOM | 16116 | CE1 | HIS | E | 210 | 62.855 | −17.880 | −38.397 | 1.00 | 56.19 | E |
| ATOM | 16117 | NE2 | HIS | E | 210 | 62.156 | −18.173 | −37.314 | 1.00 | 56.19 | E |
| ATOM | 16118 | C | HIS | E | 210 | 65.376 | −22.990 | −38.604 | 1.00 | 52.94 | E |
| ATOM | 16119 | O | HIS | E | 210 | 66.565 | −22.653 | −38.628 | 1.00 | 52.94 | E |
| ATOM | 16120 | N | THR | E | 211 | 64.975 | −24.254 | −38.662 | 1.00 | 58.28 | E |
| ATOM | 16121 | CA | THR | E | 211 | 65.944 | −25.319 | −38.847 | 1.00 | 58.28 | E |
| ATOM | 16122 | CB | THR | E | 211 | 65.440 | −26.665 | −38.297 | 1.00 | 50.98 | E |
| ATOM | 16123 | OG1 | THR | E | 211 | 65.250 | −26.563 | −36.882 | 1.00 | 50.98 | E |
| ATOM | 16124 | CG2 | THR | E | 211 | 66.454 | −27.764 | −38.578 | 1.00 | 50.98 | E |
| ATOM | 16125 | C | THR | E | 211 | 66.024 | −25.376 | −40.371 | 1.00 | 58.28 | E |
| ATOM | 16126 | O | THR | E | 211 | 65.000 | −25.481 | −41.043 | 1.00 | 58.28 | E |
| ATOM | 16127 | N | PRO | E | 212 | 67.236 | −25.279 | −40.935 | 1.00 | 48.74 | E |
| ATOM | 16128 | CD | PRO | E | 212 | 68.522 | −25.252 | −40.210 | 1.00 | 42.83 | E |
| ATOM | 16129 | CA | PRO | E | 212 | 67.445 | −25.312 | −42.388 | 1.00 | 48.74 | E |
| ATOM | 16130 | CB | PRO | E | 212 | 68.957 | −25.484 | −42.505 | 1.00 | 42.83 | E |
| ATOM | 16131 | CG | PRO | E | 212 | 69.474 | −24.776 | −41.268 | 1.00 | 42.83 | E |
| ATOM | 16132 | C | PRO | E | 212 | 66.677 | −26.412 | −43.144 | 1.00 | 48.74 | E |
| ATOM | 16133 | O | PRO | E | 212 | 66.559 | −27.540 | −42.663 | 1.00 | 48.74 | E |
| ATOM | 16134 | N | GLY | E | 213 | 66.171 | −26.072 | −44.330 | 1.00 | 56.48 | E |
| ATOM | 16135 | CA | GLY | E | 213 | 65.436 | −27.021 | −45.150 | 1.00 | 56.48 | E |
| ATOM | 16136 | C | GLY | E | 213 | 66.353 | −27.877 | −46.009 | 1.00 | 56.48 | E |
| ATOM | 16137 | O | GLY | E | 213 | 67.572 | −27.705 | −45.977 | 1.00 | 56.48 | E |
| ATOM | 16138 | N | ASN | E | 214 | 65.775 | −28.795 | −46.781 | 1.00 | 87.55 | E |
| ATOM | 16139 | CA | ASN | E | 214 | 66.551 | −29.699 | −47.630 | 1.00 | 87.55 | E |
| ATOM | 16140 | CB | ASN | E | 214 | 67.387 | −28.940 | −48.654 | 1.00 | 81.88 | E |
| ATOM | 16141 | CG | ASN | E | 214 | 66.665 | −27.765 | −49.236 | 1.00 | 76.05 | E |
| ATOM | 16142 | OD1 | ASN | E | 214 | 65.451 | −27.641 | −49.107 | 1.00 | 76.05 | E |
| ATOM | 16143 | ND2 | ASN | E | 214 | 67.409 | −26.888 | −49.893 | 1.00 | 76.05 | E |
| ATOM | 16144 | C | ASN | E | 214 | 67.501 | −30.484 | −46.752 | 1.00 | 87.55 | E |
| ATOM | 16145 | O | ASN | E | 214 | 68.699 | −30.557 | −47.034 | 1.00 | 87.55 | E |
| ATOM | 16146 | N | ASP | E | 215 | 66.967 | −31.068 | −45.687 | 1.00 | 65.76 | E |
| ATOM | 16147 | CA | ASP | E | 215 | 67.783 | −31.834 | −44.765 | 1.00 | 65.76 | E |
| ATOM | 16148 | CB | ASP | E | 215 | 68.142 | −33.190 | −45.366 | 1.00 | 183.20 | E |
| ATOM | 16149 | CG | ASP | E | 215 | 66.921 | −33.988 | −45.747 | 1.00 | 104.58 | E |
| ATOM | 16150 | OD1 | ASP | E | 215 | 66.058 | −34.207 | −44.870 | 1.00 | 104.58 | E |
| ATOM | 16151 | OD2 | ASP | E | 215 | 66.822 | −34.390 | −46.925 | 1.00 | 104.58 | E |
| ATOM | 16152 | C | ASP | E | 215 | 69.038 | −31.043 | −44.467 | 1.00 | 65.76 | E |
| ATOM | 16153 | O | ASP | E | 215 | 70.145 | −31.459 | −44.819 | 1.00 | 65.76 | E |
| ATOM | 16154 | N | TYR | E | 216 | 68.839 | −29.880 | −43.851 | 1.00 | 40.40 | E |
| ATOM | 16155 | CA | TYR | E | 216 | 69.932 | −28.989 | −43.448 | 1.00 | 40.40 | E |
| ATOM | 16156 | CB | TYR | E | 216 | 70.726 | −29.626 | −42.305 | 1.00 | 64.68 | E |
| ATOM | 16157 | CG | TYR | E | 216 | 69.875 | −30.086 | −41.154 | 1.00 | 43.43 | E |
| ATOM | 16158 | CD1 | TYR | E | 216 | 68.997 | −31.166 | −41.288 | 1.00 | 43.43 | E |
| ATOM | 16159 | CE1 | TYR | E | 216 | 68.203 | −31.597 | −40.216 | 1.00 | 43.43 | E |
| ATOM | 16160 | CD2 | TYR | E | 216 | 69.944 | −29.441 | −39.921 | 1.00 | 43.43 | E |
| ATOM | 16161 | CE2 | TYR | E | 216 | 69.159 | −29.851 | −38.836 | 1.00 | 43.43 | E |
| ATOM | 16162 | CZ | TYR | E | 216 | 68.292 | −30.931 | −38.991 | 1.00 | 43.43 | E |
| ATOM | 16163 | OH | TYR | E | 216 | 67.529 | −31.335 | −37.920 | 1.00 | 43.43 | E |
| ATOM | 16164 | C | TYR | E | 216 | 70.895 | −28.627 | −44.571 | 1.00 | 40.40 | E |
| ATOM | 16165 | O | TYR | E | 216 | 72.094 | −28.469 | −44.337 | 1.00 | 40.40 | E |
| ATOM | 16166 | N | ASN | E | 217 | 70.376 | −28.484 | −45.781 | 1.00 | 36.13 | E |
| ATOM | 16167 | CA | ASN | E | 217 | 71.219 | −28.152 | −46.927 | 1.00 | 36.13 | E |
| ATOM | 16168 | CB | ASN | E | 217 | 71.746 | −26.720 | −46.801 | 1.00 | 44.37 | E |
| ATOM | 16169 | CG | ASN | E | 217 | 70.627 | −25.683 | −46.783 | 1.00 | 39.10 | E |
| ATOM | 16170 | OD1 | ASN | E | 217 | 70.529 | −24.883 | −45.852 | 1.00 | 39.10 | E |
| ATOM | 16171 | ND2 | ASN | E | 217 | 69.777 | −25.694 | −47.822 | 1.00 | 39.10 | E |
| ATOM | 16172 | C | ASN | E | 217 | 72.399 | −29.118 | −47.022 | 1.00 | 36.13 | E |
| ATOM | 16173 | O | ASN | E | 217 | 73.498 | −28.723 | −47.435 | 1.00 | 36.13 | E |
| ATOM | 16174 | N | PHE | E | 218 | 72.160 | −30.379 | −46.659 | 1.00 | 33.21 | E |
| ATOM | 16175 | CA | PHE | E | 218 | 73.210 | −31.367 | −46.666 | 1.00 | 33.21 | E |
| ATOM | 16176 | CB | PHE | E | 218 | 72.700 | −32.714 | −46.165 | 1.00 | 43.16 | E |
| ATOM | 16177 | CG | PHE | E | 218 | 73.777 | −33.744 | −46.053 | 1.00 | 39.68 | E |
| ATOM | 16178 | CD1 | PHE | E | 218 | 74.596 | −33.782 | −44.936 | 1.00 | 39.68 | E |
| ATOM | 16179 | CD2 | PHE | E | 218 | 74.030 | −34.624 | −47.100 | 1.00 | 39.68 | E |
| ATOM | 16180 | CE1 | PHE | E | 218 | 75.656 | −34.679 | −44.858 | 1.00 | 39.68 | E |
| ATOM | 16181 | CE2 | PHE | E | 218 | 75.079 | −35.516 | −47.033 | 1.00 | 39.68 | E |
| ATOM | 16182 | CZ | PHE | E | 218 | 75.898 | −35.544 | −45.904 | 1.00 | 39.68 | E |
| ATOM | 16183 | C | PHE | E | 218 | 73.877 | −31.565 | −48.015 | 1.00 | 33.21 | E |
| ATOM | 16184 | O | PHE | E | 218 | 75.106 | −31.505 | −48.115 | 1.00 | 33.21 | E |
| ATOM | 16185 | N | ASN | E | 219 | 73.090 | −31.817 | −49.053 | 1.00 | 44.37 | E |
| ATOM | 16186 | CA | ASN | E | 219 | 73.683 | −32.030 | −50.365 | 1.00 | 44.37 | E |
| ATOM | 16187 | CB | ASN | E | 219 | 72.604 | −32.234 | −51.422 | 1.00 | 63.10 | E |
| ATOM | 16188 | CG | ASN | E | 219 | 71.683 | −33.393 | −51.095 | 1.00 | 54.73 | E |
| ATOM | 16189 | OD1 | ASN | E | 219 | 72.111 | −34.413 | −50.552 | 1.00 | 54.73 | E |
| ATOM | 16190 | ND2 | ASN | E | 219 | 70.410 | −33.246 | −51.437 | 1.00 | 54.73 | E |
| ATOM | 16191 | C | ASN | E | 219 | 74.562 | −30.852 | −50.735 | 1.00 | 44.37 | E |
| ATOM | 16192 | O | ASN | E | 219 | 75.667 | −31.022 | −51.247 | 1.00 | 44.37 | E |
| ATOM | 16193 | N | PHE | E | 220 | 74.082 | −29.648 | −50.459 | 1.00 | 44.87 | E |
| ATOM | 16194 | CA | PHE | E | 220 | 74.863 | −28.456 | −50.766 | 1.00 | 44.87 | E |
| ATOM | 16195 | CB | PHE | E | 220 | 74.114 | −27.198 | −50.337 | 1.00 | 34.05 | E |
| ATOM | 16196 | CG | PHE | E | 220 | 74.963 | −25.958 | −50.346 | 1.00 | 27.03 | E |
| ATOM | 16197 | CD1 | PHE | E | 220 | 75.170 | −25.243 | −51.514 | 1.00 | 27.03 | E |
| ATOM | 16198 | CD2 | PHE | E | 220 | 75.615 | −25.539 | −49.176 | 1.00 | 27.03 | E |
| ATOM | 16199 | CE1 | PHE | E | 220 | 76.025 | −24.120 | −51.528 | 1.00 | 27.03 | E |
| ATOM | 16200 | CE2 | PHE | E | 220 | 76.466 | −24.433 | −49.175 | 1.00 | 27.03 | E |
| ATOM | 16201 | CZ | PHE | E | 220 | 76.672 | −23.720 | −50.361 | 1.00 | 27.03 | E |
| ATOM | 16202 | C | PHE | E | 220 | 76.214 | −28.486 | −50.066 | 1.00 | 44.87 | E |
| ATOM | 16203 | O | PHE | E | 220 | 77.253 | −28.368 | −50.701 | 1.00 | 44.87 | E |
| ATOM | 16204 | N | TRP | E | 221 | 76.194 | −28.636 | −48.748 | 1.00 | 46.82 | E |
| ATOM | 16205 | CA | TRP | E | 221 | 77.428 | −28.651 | −47.976 | 1.00 | 46.82 | E |
| ATOM | 16206 | CB | TRP | E | 221 | 77.111 | −28.627 | −46.475 | 1.00 | 41.92 | E |
| ATOM | 16207 | CG | TRP | E | 221 | 76.621 | −27.300 | −46.005 | 1.00 | 35.07 | E |
| ATOM | 16208 | CD2 | TRP | E | 221 | 77.335 | −26.060 | −46.056 | 1.00 | 35.07 | E |
| ATOM | 16209 | CE2 | TRP | E | 221 | 76.467 | −25.057 | −45.573 | 1.00 | 35.07 | E |
| ATOM | 16210 | CE3 | TRP | E | 221 | 78.623 | −25.698 | −46.469 | 1.00 | 35.07 | E |
| ATOM | 16211 | CD1 | TRP | E | 221 | 75.384 | −27.010 | −45.501 | 1.00 | 35.07 | E |
| ATOM | 16212 | NE1 | TRP | E | 221 | 75.284 | −25.664 | −45.240 | 1.00 | 35.07 | E |
| ATOM | 16213 | CZ2 | TRP | E | 221 | 76.847 | −23.709 | −45.489 | 1.00 | 35.07 | E |
| ATOM | 16214 | CZ3 | TRP | E | 221 | 79.002 | −24.352 | −46.385 | 1.00 | 35.07 | E |
| ATOM | 16215 | CH2 | TRP | E | 221 | 78.115 | −23.377 | −45.899 | 1.00 | 35.07 | E |
| ATOM | 16216 | C | TRP | E | 221 | 78.344 | −29.825 | −48.300 | 1.00 | 46.82 | E |
| ATOM | 16217 | O | TRP | E | 221 | 79.549 | −29.642 | −48.502 | 1.00 | 46.82 | E |
| ATOM | 16218 | N | TYR | E | 222 | 77.787 | −31.030 | −48.345 | 1.00 | 47.06 | E |
| ATOM | 16219 | CA | TYR | E | 222 | 78.597 | −32.196 | −48.649 | 1.00 | 47.06 | E |
| ATOM | 16220 | CB | TYR | E | 222 | 77.723 | −33.413 | −48.914 | 1.00 | 46.64 | E |
| ATOM | 16221 | CG | TYR | E | 222 | 78.529 | −34.565 | −49.448 | 1.00 | 45.21 | E |
| ATOM | 16222 | CD1 | TYR | E | 222 | 79.325 | −35.339 | −48.604 | 1.00 | 45.21 | E |
| ATOM | 16223 | CE1 | TYR | E | 222 | 80.125 | −36.347 | −49.109 | 1.00 | 45.21 | E |
| ATOM | 16224 | CD2 | TYR | E | 222 | 78.556 | −34.835 | −50.808 | 1.00 | 45.21 | E |
| ATOM | 16225 | CE2 | TYR | E | 222 | 79.353 | −35.837 | −51.323 | 1.00 | 45.21 | E |
| ATOM | 16226 | CZ | TYR | E | 222 | 80.133 | −36.589 | −50.478 | 1.00 | 45.21 | E |
| ATOM | 16227 | OH | TYR | E | 222 | 80.928 | −37.570 | −51.024 | 1.00 | 45.21 | E |
| ATOM | 16228 | C | TYR | E | 222 | 79.428 | −31.924 | −49.889 | 1.00 | 47.06 | E |
| ATOM | 16229 | O | TYR | E | 222 | 80.651 | −32.052 | −49.889 | 1.00 | 47.06 | E |
| ATOM | 16230 | N | SER | E | 223 | 78.744 | −31.538 | −50.950 | 1.00 | 44.85 | E |
| ATOM | 16231 | CA | SER | E | 223 | 79.392 | −31.249 | −52.211 | 1.00 | 44.85 | E |
| ATOM | 16232 | CB | SER | E | 223 | 78.335 | −30.893 | −53.239 | 1.00 | 38.53 | E |
| ATOM | 16233 | OG | SER | E | 223 | 78.963 | −30.479 | −54.424 | 1.00 | 37.15 | E |
| ATOM | 16234 | C | SER | E | 223 | 80.446 | −30.140 | −52.164 | 1.00 | 44.85 | E |
| ATOM | 16235 | O | SER | E | 223 | 81.558 | −30.306 | −52.659 | 1.00 | 44.85 | E |
| ATOM | 16236 | N | LEU | E | 224 | 80.093 | −29.005 | −51.571 | 1.00 | 43.65 | E |
| ATOM | 16237 | CA | LEU | E | 224 | 81.010 | −27.887 | −51.483 | 1.00 | 43.65 | E |
| ATOM | 16238 | CB | LEU | E | 224 | 80.277 | −26.640 | −51.011 | 1.00 | 66.90 | E |
| ATOM | 16239 | CG | LEU | E | 224 | 81.171 | −25.404 | −50.895 | 1.00 | 44.14 | E |
| ATOM | 16240 | CD1 | LEU | E | 224 | 81.892 | −25.148 | −52.212 | 1.00 | 44.14 | E |
| ATOM | 16241 | CD2 | LEU | E | 224 | 80.325 | −24.208 | −50.496 | 1.00 | 44.14 | E |
| ATOM | 16242 | C | LEU | E | 224 | 82.171 | −28.184 | −50.556 | 1.00 | 43.65 | E |
| ATOM | 16243 | O | LEU | E | 224 | 83.273 | −27.682 | −50.758 | 1.00 | 43.65 | E |
| ATOM | 16244 | N | MET | E | 225 | 81.929 | −28.994 | −49.532 | 1.00 | 38.46 | E |
| ATOM | 16245 | CA | MET | E | 225 | 82.995 | −29.346 | −48.613 | 1.00 | 38.46 | E |
| ATOM | 16246 | CB | MET | E | 225 | 82.422 | −29.925 | −47.334 | 1.00 | 85.20 | E |
| ATOM | 16247 | CG | MET | E | 225 | 81.684 | −28.898 | −46.546 | 1.00 | 53.92 | E |
| ATOM | 16248 | SD | MET | E | 225 | 81.197 | −29.470 | −44.934 | 1.00 | 53.92 | E |
| ATOM | 16249 | CE | MET | E | 225 | 82.827 | −29.775 | −44.182 | 1.00 | 53.92 | E |
| ATOM | 16250 | C | MET | E | 225 | 83.901 | −30.361 | −49.283 | 1.00 | 38.46 | E |
| ATOM | 16251 | O | MET | E | 225 | 85.112 | −30.349 | −49.093 | 1.00 | 38.46 | E |
| ATOM | 16252 | N | LYS | E | 226 | 83.308 | −31.244 | −50.070 | 1.00 | 54.56 | E |
| ATOM | 16253 | CA | LYS | E | 226 | 84.085 | −32.243 | −50.772 | 1.00 | 54.56 | E |
| ATOM | 16254 | CB | LYS | E | 226 | 83.172 | −33.125 | −51.609 | 1.00 | 104.67 | E |
| ATOM | 16255 | CG | LYS | E | 226 | 83.897 | −34.174 | −52.392 | 1.00 | 56.13 | E |
| ATOM | 16256 | CD | LYS | E | 226 | 82.949 | −34.907 | −53.303 | 1.00 | 56.13 | E |
| ATOM | 16257 | CE | LYS | E | 226 | 83.704 | −35.801 | −54.263 | 1.00 | 56.13 | E |
| ATOM | 16258 | NZ | LYS | E | 226 | 82.787 | −36.490 | −55.211 | 1.00 | 56.13 | E |
| ATOM | 16259 | C | LYS | E | 226 | 85.058 | −31.489 | −51.668 | 1.00 | 54.56 | E |
| ATOM | 16260 | O | LYS | E | 226 | 86.248 | −31.805 | −51.706 | 1.00 | 54.56 | E |
| ATOM | 16261 | N | GLU | E | 227 | 84.548 | −30.476 | −52.369 | 1.00 | 54.53 | E |
| ATOM | 16262 | CA | GLU | E | 227 | 85.368 | −29.660 | −53.264 | 1.00 | 54.53 | E |
| ATOM | 16263 | CB | GLU | E | 227 | 84.518 | −28.575 | −53.921 | 1.00 | 122.64 | E |
| ATOM | 16264 | CG | GLU | E | 227 | 85.271 | −27.770 | −54.959 | 1.00 | 98.44 | E |
| ATOM | 16265 | CD | GLU | E | 227 | 84.551 | −26.494 | −55.350 | 1.00 | 98.44 | E |
| ATOM | 16266 | OE1 | GLU | E | 227 | 83.352 | −26.565 | −55.708 | 1.00 | 98.44 | E |
| ATOM | 16267 | OE2 | GLU | E | 227 | 85.190 | −25.420 | −55.302 | 1.00 | 98.44 | E |
| ATOM | 16268 | C | GLU | E | 227 | 86.507 | −28.999 | −52.482 | 1.00 | 54.53 | E |
| ATOM | 16269 | O | GLU | E | 227 | 87.669 | −29.031 | −52.891 | 1.00 | 54.53 | E |
| ATOM | 16270 | N | THR | E | 228 | 86.162 | −28.408 | −51.345 | 1.00 | 48.89 | E |
| ATOM | 16271 | CA | THR | E | 228 | 87.138 | −27.729 | −50.507 | 1.00 | 48.89 | E |
| ATOM | 16272 | CB | THR | E | 228 | 86.460 | −27.071 | −49.294 | 1.00 | 59.69 | E |
| ATOM | 16273 | OG1 | THR | E | 228 | 85.477 | −26.132 | −49.747 | 1.00 | 45.68 | E |
| ATOM | 16274 | CG2 | THR | E | 228 | 87.494 | −26.358 | −48.432 | 1.00 | 45.68 | E |
| ATOM | 16275 | C | THR | E | 228 | 88.219 | −28.664 | −49.992 | 1.00 | 48.89 | E |
| ATOM | 16276 | O | THR | E | 228 | 89.388 | −28.292 | −49.914 | 1.00 | 48.89 | E |
| ATOM | 16277 | N | LEU | E | 229 | 87.827 | −29.875 | −49.622 | 1.00 | 61.14 | E |
| ATOM | 16278 | CA | LEU | E | 229 | 88.789 | −30.833 | −49.111 | 1.00 | 61.14 | E |
| ATOM | 16279 | CB | LEU | E | 229 | 88.066 | −32.063 | −48.572 | 1.00 | 59.43 | E |
| ATOM | 16280 | CG | LEU | E | 229 | 88.954 | −33.142 | −47.971 | 1.00 | 31.31 | E |
| ATOM | 16281 | CD1 | LEU | E | 229 | 90.032 | −32.529 | −47.068 | 1.00 | 31.31 | E |
| ATOM | 16282 | CD2 | LEU | E | 229 | 88.073 | −34.106 | −47.187 | 1.00 | 31.31 | E |
| ATOM | 16283 | C | LEU | E | 229 | 89.777 | −31.222 | −50.205 | 1.00 | 61.14 | E |
| ATOM | 16284 | O | LEU | E | 229 | 90.974 | −31.332 | −49.943 | 1.00 | 61.14 | E |
| ATOM | 16285 | N | GLU | E | 230 | 89.279 | −31.412 | −51.429 | 1.00 | 71.28 | E |
| ATOM | 16286 | CA | GLU | E | 230 | 90.136 | −31.771 | −52.554 | 1.00 | 71.28 | E |
| ATOM | 16287 | CB | GLU | E | 230 | 89.297 | −32.006 | −53.804 | 1.00 | 146.60 | E |
| ATOM | 16288 | CG | GLU | E | 230 | 88.465 | −33.266 | −53.730 | 1.00 | 111.06 | E |
| ATOM | 16289 | CD | GLU | E | 230 | 87.682 | −33.524 | −55.001 | 1.00 | 111.06 | E |
| ATOM | 16290 | OE1 | GLU | E | 230 | 88.299 | −33.509 | −56.086 | 1.00 | 111.06 | E |
| ATOM | 16291 | OE2 | GLU | E | 230 | 86.454 | −33.751 | −54.919 | 1.00 | 111.06 | E |
| ATOM | 16292 | C | GLU | E | 230 | 91.185 | −30.692 | −52.819 | 1.00 | 71.28 | E |
| ATOM | 16293 | O | GLU | E | 230 | 92.367 | −30.994 | −52.980 | 1.00 | 71.28 | E |
| ATOM | 16294 | N | GLU | E | 231 | 90.752 | −29.434 | −52.854 | 1.00 | 59.94 | E |
| ATOM | 16295 | CA | GLU | E | 231 | 91.661 | −28.308 | −53.071 | 1.00 | 59.94 | E |
| ATOM | 16296 | CB | GLU | E | 231 | 90.882 | −26.994 | −53.096 | 1.00 | 129.36 | E |
| ATOM | 16297 | CG | GLU | E | 231 | 89.759 | −26.957 | −54.113 | 1.00 | 105.60 | E |
| ATOM | 16298 | CD | GLU | E | 231 | 90.256 | −26.993 | −55.547 | 1.00 | 105.60 | E |
| ATOM | 16299 | OE1 | GLU | E | 231 | 91.018 | −27.917 | −55.898 | 1.00 | 105.60 | E |
| ATOM | 16300 | OE2 | GLU | E | 231 | 89.877 | −26.096 | −56.329 | 1.00 | 105.60 | E |
| ATOM | 16301 | C | GLU | E | 231 | 92.695 | −28.256 | −51.955 | 1.00 | 59.94 | E |
| ATOM | 16302 | O | GLU | E | 231 | 93.882 | −28.112 | −52.210 | 1.00 | 59.94 | E |
| ATOM | 16303 | N | ILE | E | 232 | 92.232 | −28.375 | −50.717 | 1.00 | 59.16 | E |
| ATOM | 16304 | CA | ILE | E | 232 | 93.118 | −28.350 | −49.562 | 1.00 | 59.16 | E |
| ATOM | 16305 | CB | ILE | E | 232 | 92.324 | −28.562 | −48.242 | 1.00 | 70.04 | E |
| ATOM | 16306 | CG2 | ILE | E | 232 | 93.266 | −28.893 | −47.088 | 1.00 | 40.09 | E |
| ATOM | 16307 | CG1 | ILE | E | 232 | 91.515 | −27.308 | −47.912 | 1.00 | 40.09 | E |
| ATOM | 16308 | CD1 | ILE | E | 232 | 90.705 | −27.431 | −46.626 | 1.00 | 40.09 | E |
| ATOM | 16309 | C | ILE | E | 232 | 94.205 | −29.416 | −49.652 | 1.00 | 59.16 | E |
| ATOM | 16310 | O | ILE | E | 232 | 95.328 | −29.188 | −49.205 | 1.00 | 59.16 | E |
| ATOM | 16311 | N | ASN | E | 233 | 93.881 | −30.571 | −50.232 | 1.00 | 89.53 | E |
| ATOM | 16312 | CA | ASN | E | 233 | 94.853 | −31.648 | −50.336 | 1.00 | 89.53 | E |
| ATOM | 16313 | CB | ASN | E | 233 | 94.147 | −32.996 | −50.433 | 1.00 | 84.55 | E |
| ATOM | 16314 | CG | ASN | E | 233 | 93.490 | −33.398 | −49.135 | 1.00 | 73.47 | E |
| ATOM | 16315 | OD1 | ASN | E | 233 | 93.977 | −33.076 | −48.057 | 1.00 | 73.47 | E |
| ATOM | 16316 | ND2 | ASN | E | 233 | 92.385 | −34.124 | −49.231 | 1.00 | 73.47 | E |
| ATOM | 16317 | C | ASN | E | 233 | 95.869 | −31.528 | −51.467 | 1.00 | 89.53 | E |
| ATOM | 16318 | O | ASN | E | 233 | 96.842 | −32.287 | −51.497 | 1.00 | 89.53 | E |
| ATOM | 16319 | N | LYS | E | 234 | 95.662 | −30.592 | −52.392 | 1.00 | 84.06 | E |
| ATOM | 16320 | CA | LYS | E | 234 | 96.616 | −30.423 | −53.489 | 1.00 | 84.06 | E |
| ATOM | 16321 | CB | LYS | E | 234 | 96.060 | −29.478 | −54.546 | 1.00 | 71.46 | E |
| ATOM | 16322 | CG | LYS | E | 234 | 94.967 | −30.129 | −55.353 | 1.00 | 71.46 | E |
| ATOM | 16323 | CD | LYS | E | 234 | 94.428 | −29.238 | −56.443 | 1.00 | 71.46 | E |
| ATOM | 16324 | CE | LYS | E | 234 | 93.371 | −29.981 | −57.255 | 1.00 | 71.46 | E |
| ATOM | 16325 | NZ | LYS | E | 234 | 92.676 | −29.093 | −58.238 | 1.00 | 71.46 | E |
| ATOM | 16326 | C | LYS | E | 234 | 97.977 | −29.943 | −52.982 | 1.00 | 84.06 | E |
| ATOM | 16327 | O | LYS | E | 234 | 99.010 | −30.453 | −53.415 | 1.00 | 84.06 | E |
| ATOM | 16328 | N | ASN | E | 235 | 97.987 | −28.961 | −52.082 | 1.00 | 87.19 | E |
| ATOM | 16329 | CA | ASN | E | 235 | 99.249 | −28.509 | −51.502 | 1.00 | 87.19 | E |
| ATOM | 16330 | CB | ASN | E | 235 | 99.107 | −27.148 | −50.800 | 1.00 | 124.63 | E |
| ATOM | 16331 | CG | ASN | E | 235 | 100.382 | −26.738 | −50.064 | 1.00 | 124.63 | E |
| ATOM | 16332 | OD1 | ASN | E | 235 | 100.988 | −27.563 | −49.382 | 1.00 | 124.63 | E |
| ATOM | 16333 | ND2 | ASN | E | 235 | 100.800 | −25.479 | −50.190 | 1.00 | 124.63 | E |
| ATOM | 16334 | C | ASN | E | 235 | 99.516 | −29.604 | −50.471 | 1.00 | 87.19 | E |
| ATOM | 16335 | O | ASN | E | 235 | 98.824 | −29.692 | −49.456 | 1.00 | 87.19 | E |
| ATOM | 16336 | N | ASP | E | 236 | 100.512 | −30.441 | −50.740 | 1.00 | 100.54 | E |
| ATOM | 16337 | CA | ASP | E | 236 | 100.839 | −31.559 | −49.862 | 1.00 | 100.54 | E |
| ATOM | 16338 | CB | ASP | E | 236 | 101.960 | −32.388 | −50.490 | 1.00 | 140.73 | E |
| ATOM | 16339 | CG | ASP | E | 236 | 101.948 | −33.827 | −50.022 | 1.00 | 140.73 | E |
| ATOM | 16340 | OD1 | ASP | E | 236 | 102.273 | −34.078 | −48.843 | 1.00 | 140.73 | E |
| ATOM | 16341 | OD2 | ASP | E | 236 | 101.600 | −34.707 | −50.837 | 1.00 | 140.73 | E |
| ATOM | 16342 | C | ASP | E | 236 | 101.200 | −31.213 | −48.414 | 1.00 | 100.54 | E |
| ATOM | 16343 | O | ASP | E | 236 | 101.361 | −32.109 | −47.585 | 1.00 | 100.54 | E |
| ATOM | 16344 | N | SER | E | 237 | 101.319 | −29.927 | −48.102 | 1.00 | 99.72 | E |
| ATOM | 16345 | CA | SER | E | 237 | 101.651 | −29.521 | −46.737 | 1.00 | 99.72 | E |
| ATOM | 16346 | CB | SER | E | 237 | 102.562 | −28.288 | −46.739 | 1.00 | 112.89 | E |
| ATOM | 16347 | OG | SER | E | 237 | 101.832 | −27.112 | −47.039 | 1.00 | 112.89 | E |
| ATOM | 16348 | C | SER | E | 237 | 100.399 | −29.212 | −45.918 | 1.00 | 99.72 | E |
| ATOM | 16349 | O | SER | E | 237 | 100.494 | −28.783 | −44.768 | 1.00 | 99.72 | E |
| ATOM | 16350 | N | THR | E | 238 | 99.230 | −29.428 | −46.517 | 1.00 | 112.32 | E |
| ATOM | 16351 | CA | THR | E | 238 | 97.958 | −29.185 | −45.840 | 1.00 | 112.32 | E |
| ATOM | 16352 | CB | THR | E | 238 | 97.129 | −28.120 | −46.566 | 1.00 | 106.98 | E |
| ATOM | 16353 | OG1 | THR | E | 238 | 97.969 | −27.016 | −46.913 | 1.00 | 106.98 | E |
| ATOM | 16354 | CG2 | THR | E | 238 | 96.005 | −27.627 | −45.666 | 1.00 | 106.98 | E |
| ATOM | 16355 | C | THR | E | 238 | 97.132 | −30.465 | −45.798 | 1.00 | 112.32 | E |
| ATOM | 16356 | O | THR | E | 238 | 96.266 | −30.625 | −44.937 | 1.00 | 112.32 | E |
| ATOM | 16357 | N | LYS | E | 239 | 97.410 | −31.364 | −46.741 | 1.00 | 94.22 | E |
| ATOM | 16358 | CA | LYS | E | 239 | 96.719 | −32.647 | −46.857 | 1.00 | 94.22 | E |
| ATOM | 16359 | CB | LYS | E | 239 | 97.692 | −33.723 | −47.357 | 1.00 | 99.38 | E |
| ATOM | 16360 | CG | LYS | E | 239 | 97.058 | −35.089 | −47.575 | 1.00 | 99.38 | E |
| ATOM | 16361 | CD | LYS | E | 239 | 98.000 | −36.018 | −48.314 | 1.00 | 99.38 | E |
| ATOM | 16362 | CE | LYS | E | 239 | 98.324 | −35.475 | −49.699 | 1.00 | 99.38 | E |
| ATOM | 16363 | NZ | LYS | E | 239 | 99.242 | −36.379 | −50.442 | 1.00 | 99.38 | E |
| ATOM | 16364 | C | LYS | E | 239 | 96.089 | −33.082 | −45.539 | 1.00 | 94.22 | E |
| ATOM | 16365 | O | LYS | E | 239 | 96.774 | −33.207 | −44.524 | 1.00 | 94.22 | E |
| ATOM | 16366 | N | THR | E | 240 | 94.779 | −33.305 | −45.566 | 1.00 | 56.81 | E |
| ATOM | 16367 | CA | THR | E | 240 | 94.048 | −33.707 | −44.376 | 1.00 | 56.81 | E |
| ATOM | 16368 | CB | THR | E | 240 | 93.217 | −32.540 | −43.813 | 1.00 | 104.13 | E |
| ATOM | 16369 | OG1 | THR | E | 240 | 94.061 | −31.400 | −43.622 | 1.00 | 104.13 | E |
| ATOM | 16370 | CG2 | THR | E | 240 | 92.592 | −32.930 | −42.480 | 1.00 | 104.13 | E |
| ATOM | 16371 | C | THR | E | 240 | 93.094 | −34.861 | −44.644 | 1.00 | 56.81 | E |
| ATOM | 16372 | O | THR | E | 240 | 92.623 | −35.048 | −45.762 | 1.00 | 56.81 | E |
| ATOM | 16373 | N | THR | E | 241 | 92.813 | −35.619 | −43.591 | 1.00 | 49.74 | E |
| ATOM | 16374 | CA | THR | E | 241 | 91.901 | −36.758 | −43.639 | 1.00 | 49.74 | E |
| ATOM | 16375 | CB | THR | E | 241 | 91.934 | −37.510 | −42.312 | 1.00 | 106.61 | E |
| ATOM | 16376 | OG1 | THR | E | 241 | 93.291 | −37.765 | −41.951 | 1.00 | 66.31 | E |
| ATOM | 16377 | CG2 | THR | E | 241 | 91.178 | −38.806 | −42.411 | 1.00 | 66.31 | E |
| ATOM | 16378 | C | THR | E | 241 | 90.459 | −36.282 | −43.838 | 1.00 | 49.74 | E |
| ATOM | 16379 | O | THR | E | 241 | 89.724 | −36.841 | −44.633 | 1.00 | 49.74 | E |
| ATOM | 16380 | N | SER | E | 242 | 90.065 | −35.244 | −43.100 | 1.00 | 55.41 | E |
| ATOM | 16381 | CA | SER | E | 242 | 88.705 | −34.737 | −43.161 | 1.00 | 55.41 | E |
| ATOM | 16382 | CB | SER | E | 242 | 87.853 | −35.443 | −42.114 | 1.00 | 82.01 | E |
| ATOM | 16383 | OG | SER | E | 242 | 88.340 | −35.158 | −40.818 | 1.00 | 54.53 | E |
| ATOM | 16384 | C | SER | E | 242 | 88.568 | −33.243 | −42.955 | 1.00 | 55.41 | E |
| ATOM | 16385 | O | SER | E | 242 | 89.516 | −32.557 | −42.593 | 1.00 | 55.41 | E |
| ATOM | 16386 | N | LEU | E | 243 | 87.349 | −32.766 | −43.173 | 1.00 | 48.96 | E |
| ATOM | 16387 | CA | LEU | E | 243 | 87.009 | −31.360 | −43.037 | 1.00 | 48.96 | E |
| ATOM | 16388 | CB | LEU | E | 243 | 86.916 | −30.727 | −44.428 | 1.00 | 60.50 | E |
| ATOM | 16389 | CG | LEU | E | 243 | 86.846 | −29.208 | −44.452 | 1.00 | 36.63 | E |
| ATOM | 16390 | CD1 | LEU | E | 243 | 88.089 | −28.631 | −43.785 | 1.00 | 36.63 | E |
| ATOM | 16391 | CD2 | LEU | E | 243 | 86.742 | −28.730 | −45.896 | 1.00 | 36.63 | E |
| ATOM | 16392 | C | LEU | E | 243 | 85.667 | −31.268 | −42.306 | 1.00 | 48.96 | E |
| ATOM | 16393 | O | LEU | E | 243 | 84.775 | −32.071 | −42.540 | 1.00 | 48.96 | E |
| ATOM | 16394 | N | SER | E | 244 | 85.531 | −30.301 | −41.407 | 1.00 | 45.63 | E |
| ATOM | 16395 | CA | SER | E | 244 | 84.288 | −30.142 | −40.662 | 1.00 | 45.63 | E |
| ATOM | 16396 | CB | SER | E | 244 | 84.458 | −30.633 | −39.224 | 1.00 | 41.49 | E |
| ATOM | 16397 | OG | SER | E | 244 | 84.665 | −32.028 | −39.164 | 1.00 | 43.64 | E |
| ATOM | 16398 | C | SER | E | 244 | 83.774 | −28.709 | −40.611 | 1.00 | 45.63 | E |
| ATOM | 16399 | O | SER | E | 244 | 84.549 | −27.754 | −40.503 | 1.00 | 45.63 | E |
| ATOM | 16400 | N | LEU | E | 245 | 82.457 | −28.566 | −40.701 | 1.00 | 42.55 | E |
| ATOM | 16401 | CA | LEU | E | 245 | 81.826 | −27.264 | −40.606 | 1.00 | 42.55 | E |
| ATOM | 16402 | CB | LEU | E | 245 | 80.849 | −27.047 | −41.755 | 1.00 | 51.49 | E |
| ATOM | 16403 | CG | LEU | E | 245 | 80.076 | −25.732 | −41.636 | 1.00 | 35.48 | E |
| ATOM | 16404 | CD1 | LEU | E | 245 | 81.059 | −24.574 | −41.534 | 1.00 | 35.48 | E |
| ATOM | 16405 | CD2 | LEU | E | 245 | 79.145 | −25.568 | −42.826 | 1.00 | 35.48 | E |
| ATOM | 16406 | C | LEU | E | 245 | 81.080 | −27.263 | −39.269 | 1.00 | 42.55 | E |
| ATOM | 16407 | O | LEU | E | 245 | 80.076 | −27.945 | −39.109 | 1.00 | 42.55 | E |
| ATOM | 16408 | N | ILE | E | 246 | 81.596 | −26.521 | −38.298 | 1.00 | 46.13 | E |
| ATOM | 16409 | CA | ILE | E | 246 | 80.967 | −26.447 | −36.988 | 1.00 | 46.13 | E |
| ATOM | 16410 | CB | ILE | E | 246 | 82.014 | −26.534 | −35.868 | 1.00 | 45.68 | E |
| ATOM | 16411 | CG2 | ILE | E | 246 | 81.325 | −26.541 | −34.519 | 1.00 | 28.72 | E |
| ATOM | 16412 | CG1 | ILE | E | 246 | 82.862 | −27.789 | −36.054 | 1.00 | 28.72 | E |
| ATOM | 16413 | CD1 | ILE | E | 246 | 82.052 | −29.061 | −36.131 | 1.00 | 28.72 | E |
| ATOM | 16414 | C | ILE | E | 246 | 80.227 | −25.130 | −36.841 | 1.00 | 46.13 | E |
| ATOM | 16415 | O | ILE | E | 246 | 80.827 | −24.060 | −36.915 | 1.00 | 46.13 | E |
| ATOM | 16416 | N | THR | E | 247 | 78.923 | −25.200 | −36.637 | 1.00 | 28.55 | E |
| ATOM | 16417 | CA | THR | E | 247 | 78.147 | −23.984 | −36.478 | 1.00 | 28.55 | E |
| ATOM | 16418 | CB | THR | E | 247 | 77.552 | −23.545 | −37.825 | 1.00 | 47.01 | E |
| ATOM | 16419 | OG1 | THR | E | 247 | 77.005 | −22.230 | −37.696 | 1.00 | 44.25 | E |
| ATOM | 16420 | CG2 | THR | E | 247 | 76.457 | −24.514 | −38.272 | 1.00 | 44.25 | E |
| ATOM | 16421 | C | THR | E | 247 | 77.037 | −24.247 | −35.465 | 1.00 | 28.55 | E |
| ATOM | 16422 | O | THR | E | 247 | 76.558 | −25.375 | −35.350 | 1.00 | 28.55 | E |
| ATOM | 16423 | N | GLY | E | 248 | 76.639 | −23.216 | −34.726 | 1.00 | 26.35 | E |
| ATOM | 16424 | CA | GLY | E | 248 | 75.602 | −23.380 | −33.722 | 1.00 | 26.35 | E |
| ATOM | 16425 | C | GLY | E | 248 | 75.552 | −22.200 | −32.789 | 1.00 | 26.35 | E |
| ATOM | 16426 | O | GLY | E | 248 | 76.222 | −21.202 | −33.026 | 1.00 | 26.35 | E |
| ATOM | 16427 | N | CYS | E | 249 | 74.762 | −22.300 | −31.730 | 1.00 | 43.87 | E |
| ATOM | 16428 | CA | CYS | E | 249 | 74.645 | −21.196 | −30.790 | 1.00 | 43.87 | E |
| ATOM | 16429 | CB | CYS | E | 249 | 73.481 | −20.276 | −31.188 | 1.00 | 26.91 | E |
| ATOM | 16430 | SG | CYS | E | 249 | 71.858 | −20.961 | −30.886 | 1.00 | 45.68 | E |
| ATOM | 16431 | C | CYS | E | 249 | 74.443 | −21.703 | −29.372 | 1.00 | 43.87 | E |
| ATOM | 16432 | O | CYS | E | 249 | 74.372 | −22.916 | −29.146 | 1.00 | 43.87 | E |
| ATOM | 16433 | N | TYR | E | 250 | 74.347 | −20.777 | −28.417 | 1.00 | 37.52 | E |
| ATOM | 16434 | CA | TYR | E | 250 | 74.173 | −21.162 | −27.025 | 1.00 | 37.52 | E |
| ATOM | 16435 | CB | TYR | E | 250 | 75.527 | −21.526 | −26.431 | 1.00 | 42.77 | E |
| ATOM | 16436 | CG | TYR | E | 250 | 76.497 | −20.361 | −26.436 | 1.00 | 33.12 | E |
| ATOM | 16437 | CD1 | TYR | E | 250 | 76.576 | −19.483 | −25.343 | 1.00 | 33.12 | E |
| ATOM | 16438 | CE1 | TYR | E | 250 | 77.423 | −18.387 | −25.369 | 1.00 | 33.12 | E |
| ATOM | 16439 | CD2 | TYR | E | 250 | 77.296 | −20.103 | −27.554 | 1.00 | 33.12 | E |
| ATOM | 16440 | CE2 | TYR | E | 250 | 78.144 | −19.000 | −27.591 | 1.00 | 33.12 | E |
| ATOM | 16441 | CZ | TYR | E | 250 | 78.205 | −18.148 | −26.501 | 1.00 | 33.12 | E |
| ATOM | 16442 | OH | TYR | E | 250 | 79.034 | −17.052 | −26.563 | 1.00 | 33.12 | E |
| ATOM | 16443 | C | TYR | E | 250 | 73.562 | −20.064 | −26.178 | 1.00 | 37.52 | E |
| ATOM | 16444 | O | TYR | E | 250 | 73.663 | −18.875 | −26.501 | 1.00 | 37.52 | E |
| ATOM | 16445 | N | GLU | E | 251 | 72.931 | −20.496 | −25.091 | 1.00 | 54.25 | E |
| ATOM | 16446 | CA | GLU | E | 251 | 72.327 | −19.625 | −24.102 | 1.00 | 54.25 | E |
| ATOM | 16447 | CB | GLU | E | 251 | 70.809 | −19.603 | −24.235 | 1.00 | 109.91 | E |
| ATOM | 16448 | CG | GLU | E | 251 | 70.291 | −18.257 | −24.673 | 1.00 | 63.53 | E |
| ATOM | 16449 | CD | GLU | E | 251 | 68.777 | −18.161 | −24.647 | 1.00 | 63.53 | E |
| ATOM | 16450 | OE1 | GLU | E | 251 | 68.181 | −18.420 | −23.573 | 1.00 | 63.53 | E |
| ATOM | 16451 | OE2 | GLU | E | 251 | 68.188 | −17.814 | −25.701 | 1.00 | 63.53 | E |
| ATOM | 16452 | C | GLU | E | 251 | 72.743 | −20.270 | −22.781 | 1.00 | 54.25 | E |
| ATOM | 16453 | O | GLU | E | 251 | 71.988 | −21.035 | −22.176 | 1.00 | 54.25 | E |
| ATOM | 16454 | N | THR | E | 252 | 73.976 | −19.979 | −22.369 | 1.00 | 49.09 | E |
| ATOM | 16455 | CA | THR | E | 252 | 74.558 | −20.506 | −21.137 | 1.00 | 49.09 | E |
| ATOM | 16456 | CB | THR | E | 252 | 76.063 | −20.777 | −21.324 | 1.00 | 51.56 | E |
| ATOM | 16457 | OG1 | THR | E | 252 | 76.262 | −21.600 | −22.482 | 1.00 | 51.56 | E |
| ATOM | 16458 | CG2 | THR | E | 252 | 76.627 | −21.479 | −20.109 | 1.00 | 51.56 | E |
| ATOM | 16459 | C | THR | E | 252 | 74.383 | −19.463 | −20.041 | 1.00 | 49.09 | E |
| ATOM | 16460 | O | THR | E | 252 | 75.191 | −18.547 | −19.922 | 1.00 | 49.09 | E |
| ATOM | 16461 | N | GLY | E | 253 | 73.338 | −19.604 | −19.232 | 1.00 | 51.02 | E |
| ATOM | 16462 | CA | GLY | E | 253 | 73.096 | −18.610 | −18.200 | 1.00 | 51.02 | E |
| ATOM | 16463 | C | GLY | E | 253 | 72.840 | −17.254 | −18.851 | 1.00 | 51.02 | E |
| ATOM | 16464 | O | GLY | E | 253 | 72.012 | −17.138 | −19.765 | 1.00 | 51.02 | E |
| ATOM | 16465 | N | LEU | E | 254 | 73.550 | −16.226 | −18.397 | 1.00 | 52.80 | E |
| ATOM | 16466 | CA | LEU | E | 254 | 73.397 | −14.890 | −18.971 | 1.00 | 52.80 | E |
| ATOM | 16467 | CB | LEU | E | 254 | 73.972 | −13.816 | −18.054 | 1.00 | 41.56 | E |
| ATOM | 16468 | CG | LEU | E | 254 | 73.277 | −13.525 | −16.734 | 1.00 | 41.56 | E |
| ATOM | 16469 | CD1 | LEU | E | 254 | 73.935 | −12.314 | −16.101 | 1.00 | 41.56 | E |
| ATOM | 16470 | CD2 | LEU | E | 254 | 71.806 | −13.273 | −16.971 | 1.00 | 41.56 | E |
| ATOM | 16471 | C | LEU | E | 254 | 74.101 | −14.768 | −20.311 | 1.00 | 52.80 | E |
| ATOM | 16472 | O | LEU | E | 254 | 73.699 | −13.964 | −21.148 | 1.00 | 52.80 | E |
| ATOM | 16473 | N | LEU | E | 255 | 75.161 | −15.548 | −20.504 | 1.00 | 45.06 | E |
| ATOM | 16474 | CA | LEU | E | 255 | 75.910 | −15.495 | −21.749 | 1.00 | 45.06 | E |
| ATOM | 16475 | CB | LEU | E | 255 | 77.231 | −16.231 | −21.595 | 1.00 | 34.43 | E |
| ATOM | 16476 | CG | LEU | E | 255 | 78.092 | −15.817 | −20.402 | 1.00 | 34.43 | E |
| ATOM | 16477 | CD1 | LEU | E | 255 | 79.421 | −16.571 | −20.440 | 1.00 | 34.43 | E |
| ATOM | 16478 | CD2 | LEU | E | 255 | 78.329 | −14.323 | −20.440 | 1.00 | 34.43 | E |
| ATOM | 16479 | C | LEU | E | 255 | 75.142 | −16.093 | −22.920 | 1.00 | 45.06 | E |
| ATOM | 16480 | O | LEU | E | 255 | 74.320 | −16.985 | −22.743 | 1.00 | 45.06 | E |
| ATOM | 16481 | N | PHE | E | 256 | 75.404 | −15.593 | −24.120 | 1.00 | 42.82 | E |
| ATOM | 16482 | CA | PHE | E | 256 | 74.747 | −16.101 | −25.319 | 1.00 | 42.82 | E |
| ATOM | 16483 | CB | PHE | E | 256 | 73.303 | −15.598 | −25.391 | 1.00 | 52.19 | E |
| ATOM | 16484 | CG | PHE | E | 256 | 73.178 | −14.116 | −25.569 | 1.00 | 49.20 | E |
| ATOM | 16485 | CD1 | PHE | E | 256 | 73.002 | −13.567 | −26.833 | 1.00 | 49.20 | E |
| ATOM | 16486 | CD2 | PHE | E | 256 | 73.229 | −13.263 | −24.470 | 1.00 | 49.20 | E |
| ATOM | 16487 | CE1 | PHE | E | 256 | 72.875 | −12.197 | −27.000 | 1.00 | 49.20 | E |
| ATOM | 16488 | CE2 | PHE | E | 256 | 73.104 | −11.886 | −24.627 | 1.00 | 49.20 | E |
| ATOM | 16489 | CZ | PHE | E | 256 | 72.927 | −11.352 | −25.887 | 1.00 | 49.20 | E |
| ATOM | 16490 | C | PHE | E | 256 | 75.536 | −15.671 | −26.552 | 1.00 | 42.82 | E |
| ATOM | 16491 | O | PHE | E | 256 | 76.099 | −14.576 | −26.579 | 1.00 | 42.82 | E |
| ATOM | 16492 | N | GLY | E | 257 | 75.598 | −16.546 | −27.554 | 1.00 | 45.52 | E |
| ATOM | 16493 | CA | GLY | E | 257 | 76.326 | −16.232 | −28.769 | 1.00 | 45.52 | E |
| ATOM | 16494 | C | GLY | E | 257 | 76.147 | −17.309 | −29.812 | 1.00 | 45.52 | E |
| ATOM | 16495 | O | GLY | E | 257 | 75.444 | −18.297 | −29.578 | 1.00 | 45.52 | E |
| ATOM | 16496 | N | SER | E | 258 | 76.776 | −17.129 | −30.968 | 1.00 | 32.67 | E |
| ATOM | 16497 | CA | SER | E | 258 | 76.669 | −18.103 | −32.042 | 1.00 | 32.67 | E |
| ATOM | 16498 | CB | SER | E | 258 | 75.658 | −17.631 | −33.085 | 1.00 | 34.55 | E |
| ATOM | 16499 | OG | SER | E | 258 | 76.156 | −16.509 | −33.784 | 1.00 | 44.67 | E |
| ATOM | 16500 | C | SER | E | 258 | 78.042 | −18.255 | −32.673 | 1.00 | 32.67 | E |
| ATOM | 16501 | O | SER | E | 258 | 78.906 | −17.411 | −32.478 | 1.00 | 32.67 | E |
| ATOM | 16502 | N | TYR | E | 259 | 78.252 | −19.331 | −33.420 | 1.00 | 31.70 | E |
| ATOM | 16503 | CA | TYR | E | 259 | 79.544 | −19.551 | −34.051 | 1.00 | 31.70 | E |
| ATOM | 16504 | CB | TYR | E | 259 | 80.458 | −20.328 | −33.108 | 1.00 | 44.20 | E |
| ATOM | 16505 | CG | TYR | E | 259 | 79.816 | −21.598 | −32.604 | 1.00 | 39.95 | E |
| ATOM | 16506 | CD1 | TYR | E | 259 | 79.807 | −22.764 | −33.382 | 1.00 | 39.95 | E |
| ATOM | 16507 | CE1 | TYR | E | 259 | 79.116 | −23.893 | −32.967 | 1.00 | 39.95 | E |
| ATOM | 16508 | CD2 | TYR | E | 259 | 79.122 | −21.612 | −31.393 | 1.00 | 39.95 | E |
| ATOM | 16509 | CE2 | TYR | E | 259 | 78.431 | −22.733 | −30.980 | 1.00 | 39.95 | E |
| ATOM | 16510 | CZ | TYR | E | 259 | 78.427 | −23.864 | −31.768 | 1.00 | 39.95 | E |
| ATOM | 16511 | OH | TYR | E | 259 | 77.694 | −24.950 | −31.365 | 1.00 | 39.95 | E |
| ATOM | 16512 | C | TYR | E | 259 | 79.417 | −20.296 | −35.361 | 1.00 | 31.70 | E |
| ATOM | 16513 | O | TYR | E | 259 | 78.382 | −20.890 | −35.659 | 1.00 | 31.70 | E |
| ATOM | 16514 | N | GLY | E | 260 | 80.489 | −20.262 | −36.138 | 1.00 | 36.84 | E |
| ATOM | 16515 | CA | GLY | E | 260 | 80.504 | −20.937 | −37.414 | 1.00 | 36.84 | E |
| ATOM | 16516 | C | GLY | E | 260 | 81.936 | −20.952 | −37.881 | 1.00 | 36.84 | E |
| ATOM | 16517 | O | GLY | E | 260 | 82.476 | −19.926 | −38.312 | 1.00 | 36.84 | E |
| ATOM | 16518 | N | TYR | E | 261 | 82.578 | −22.104 | −37.774 | 1.00 | 45.24 | E |
| ATOM | 16519 | CA | TYR | E | 261 | 83.956 | −22.205 | −38.204 | 1.00 | 45.24 | E |
| ATOM | 16520 | CB | TYR | E | 261 | 84.883 | −22.028 | −37.019 | 1.00 | 54.32 | E |
| ATOM | 16521 | CG | TYR | E | 261 | 84.693 | −23.056 | −35.939 | 1.00 | 38.11 | E |
| ATOM | 16522 | CD1 | TYR | E | 261 | 85.396 | −24.259 | −35.959 | 1.00 | 38.11 | E |
| ATOM | 16523 | CE1 | TYR | E | 261 | 85.285 | −25.173 | −34.920 | 1.00 | 38.11 | E |
| ATOM | 16524 | CD2 | TYR | E | 261 | 83.857 | −22.798 | −34.853 | 1.00 | 38.11 | E |
| ATOM | 16525 | CE2 | TYR | E | 261 | 83.733 | −23.707 | −33.807 | 1.00 | 38.11 | E |
| ATOM | 16526 | CZ | TYR | E | 261 | 84.453 | −24.894 | −33.844 | 1.00 | 38.11 | E |
| ATOM | 16527 | OH | TYR | E | 261 | 84.331 | −25.784 | −32.796 | 1.00 | 38.11 | E |
| ATOM | 16528 | C | TYR | E | 261 | 84.235 | −23.525 | −38.889 | 1.00 | 45.24 | E |
| ATOM | 16529 | O | TYR | E | 261 | 83.454 | −24.470 | −38.803 | 1.00 | 45.24 | E |
| ATOM | 16530 | N | VAL | E | 262 | 85.362 | −23.577 | −39.583 | 1.00 | 48.24 | E |
| ATOM | 16531 | CA | VAL | E | 262 | 85.782 | −24.765 | −40.305 | 1.00 | 48.24 | E |
| ATOM | 16532 | CB | VAL | E | 262 | 86.069 | −24.428 | −41.778 | 1.00 | 42.18 | E |
| ATOM | 16533 | CG1 | VAL | E | 262 | 86.753 | −25.600 | −42.448 | 1.00 | 33.67 | E |
| ATOM | 16534 | CG2 | VAL | E | 262 | 84.775 | −24.085 | −42.497 | 1.00 | 33.67 | E |
| ATOM | 16535 | C | VAL | E | 262 | 87.057 | −25.314 | −39.696 | 1.00 | 48.24 | E |
| ATOM | 16536 | O | VAL | E | 262 | 88.052 | −24.607 | −39.607 | 1.00 | 48.24 | E |
| ATOM | 16537 | N | GLU | E | 263 | 87.031 | −26.567 | −39.264 | 1.00 | 55.06 | E |
| ATOM | 16538 | CA | GLU | E | 263 | 88.224 | −27.186 | −38.701 | 1.00 | 55.06 | E |
| ATOM | 16539 | CB | GLU | E | 263 | 88.028 | −27.591 | −37.237 | 1.00 | 93.64 | E |
| ATOM | 16540 | CG | GLU | E | 263 | 86.852 | −28.480 | −36.983 | 1.00 | 80.24 | E |
| ATOM | 16541 | CD | GLU | E | 263 | 86.731 | −28.867 | −35.523 | 1.00 | 80.24 | E |
| ATOM | 16542 | OE1 | GLU | E | 263 | 86.956 | −28.000 | −34.641 | 1.00 | 80.24 | E |
| ATOM | 16543 | OE2 | GLU | E | 263 | 86.396 | −30.043 | −35.259 | 1.00 | 80.24 | E |
| ATOM | 16544 | C | GLU | E | 263 | 88.643 | −28.393 | −39.532 | 1.00 | 55.06 | E |
| ATOM | 16545 | O | GLU | E | 263 | 87.863 | −28.973 | −40.290 | 1.00 | 55.06 | E |
| ATOM | 16546 | N | THR | E | 264 | 89.905 | −28.750 | −39.378 | 1.00 | 63.39 | E |
| ATOM | 16547 | CA | THR | E | 264 | 90.522 | −29.839 | −40.103 | 1.00 | 63.39 | E |
| ATOM | 16548 | CB | THR | E | 264 | 91.726 | −29.272 | −40.885 | 1.00 | 78.05 | E |
| ATOM | 16549 | OG1 | THR | E | 264 | 91.339 | −29.082 | −42.243 | 1.00 | 56.98 | E |
| ATOM | 16550 | CG2 | THR | E | 264 | 92.928 | −30.160 | −40.814 | 1.00 | 56.98 | E |
| ATOM | 16551 | C | THR | E | 264 | 90.959 | −30.903 | −39.115 | 1.00 | 63.39 | E |
| ATOM | 16552 | O | THR | E | 264 | 90.973 | −30.673 | −37.904 | 1.00 | 63.39 | E |
| ATOM | 16553 | N | ALA | E | 265 | 91.300 | −32.078 | −39.617 | 1.00 | 60.61 | E |
| ATOM | 16554 | CA | ALA | E | 265 | 91.756 | −33.141 | −38.731 | 1.00 | 60.61 | E |
| ATOM | 16555 | CB | ALA | E | 265 | 92.054 | −34.405 | −39.536 | 1.00 | 53.08 | E |
| ATOM | 16556 | C | ALA | E | 265 | 93.017 | −32.658 | −38.007 | 1.00 | 60.61 | E |
| ATOM | 16557 | O | ALA | E | 265 | 93.354 | −33.151 | −36.944 | 1.00 | 60.61 | E |
| ATOM | 16558 | N | ASN | E | 266 | 93.696 | −31.680 | −38.599 | 1.00 | 71.33 | E |
| ATOM | 16559 | CA | ASN | E | 266 | 94.913 | −31.111 | −38.035 | 1.00 | 71.33 | E |
| ATOM | 16560 | CB | ASN | E | 266 | 95.800 | −30.551 | −39.144 | 1.00 | 137.07 | E |
| ATOM | 16561 | CG | ASN | E | 266 | 96.103 | −31.571 | −40.210 | 1.00 | 78.92 | E |
| ATOM | 16562 | OD1 | ASN | E | 266 | 96.548 | −32.677 | −39.914 | 1.00 | 78.92 | E |
| ATOM | 16563 | ND2 | ASN | E | 266 | 95.866 | −31.206 | −41.464 | 1.00 | 78.92 | E |
| ATOM | 16564 | C | ASN | E | 266 | 94.621 | −30.001 | −37.036 | 1.00 | 71.33 | E |
| ATOM | 16565 | O | ASN | E | 266 | 95.515 | −29.585 | −36.309 | 1.00 | 71.33 | E |
| ATOM | 16566 | N | GLY | E | 267 | 93.379 | −29.517 | −37.011 | 1.00 | 72.27 | E |
| ATOM | 16567 | CA | GLY | E | 267 | 93.013 | −28.465 | −36.078 | 1.00 | 72.27 | E |
| ATOM | 16568 | C | GLY | E | 267 | 92.212 | −27.327 | −36.687 | 1.00 | 72.27 | E |
| ATOM | 16569 | O | GLY | E | 267 | 92.030 | −27.282 | −37.899 | 1.00 | 72.27 | E |
| ATOM | 16570 | N | PRO | E | 268 | 91.733 | −26.377 | −35.864 | 1.00 | 61.72 | E |
| ATOM | 16571 | CD | PRO | E | 268 | 92.114 | −26.176 | −34.455 | 1.00 | 53.61 | E |
| ATOM | 16572 | CA | PRO | E | 268 | 90.948 | −25.241 | −36.352 | 1.00 | 61.72 | E |
| ATOM | 16573 | CB | PRO | E | 268 | 90.864 | −24.327 | −35.129 | 1.00 | 69.50 | E |
| ATOM | 16574 | CG | PRO | E | 268 | 92.091 | −24.683 | −34.347 | 1.00 | 53.61 | E |
| ATOM | 16575 | C | PRO | E | 268 | 91.594 | −24.571 | −37.550 | 1.00 | 61.72 | E |
| ATOM | 16576 | O | PRO | E | 268 | 92.804 | −24.370 | −37.588 | 1.00 | 61.72 | E |
| ATOM | 16577 | N | LEU | E | 269 | 90.767 | −24.231 | −38.530 | 1.00 | 60.13 | E |
| ATOM | 16578 | CA | LEU | E | 269 | 91.233 | −23.598 | −39.747 | 1.00 | 60.13 | E |
| ATOM | 16579 | CB | LEU | E | 269 | 90.731 | −24.402 | −40.937 | 1.00 | 85.06 | E |
| ATOM | 16580 | CG | LEU | E | 269 | 91.411 | −24.147 | −42.268 | 1.00 | 50.40 | E |
| ATOM | 16581 | CD1 | LEU | E | 269 | 92.905 | −24.316 | −42.093 | 1.00 | 50.40 | E |
| ATOM | 16582 | CD2 | LEU | E | 269 | 90.866 | −25.111 | −43.310 | 1.00 | 50.40 | E |
| ATOM | 16583 | C | LEU | E | 269 | 90.781 | −22.139 | −39.857 | 1.00 | 60.13 | E |
| ATOM | 16584 | O | LEU | E | 269 | 91.592 | −21.228 | −39.682 | 1.00 | 60.13 | E |
| ATOM | 16585 | N | ALA | E | 270 | 89.496 | −21.915 | −40.128 | 1.00 | 49.51 | E |
| ATOM | 16586 | CA | ALA | E | 270 | 88.968 | −20.555 | −40.262 | 1.00 | 49.51 | E |
| ATOM | 16587 | CB | ALA | E | 270 | 88.875 | −20.169 | −41.741 | 1.00 | 36.24 | E |
| ATOM | 16588 | C | ALA | E | 270 | 87.613 | −20.353 | −39.599 | 1.00 | 49.51 | E |
| ATOM | 16589 | O | ALA | E | 270 | 86.764 | −21.243 | −39.589 | 1.00 | 49.51 | E |
| ATOM | 16590 | N | ARG | E | 271 | 87.413 | −19.147 | −39.082 | 1.00 | 55.73 | E |
| ATOM | 16591 | CA | ARG | E | 271 | 86.188 | −18.755 | −38.394 | 1.00 | 55.73 | E |
| ATOM | 16592 | CB | ARG | E | 271 | 86.590 | −18.165 | −37.046 | 1.00 | 51.15 | E |
| ATOM | 16593 | CG | ARG | E | 271 | 85.519 | −17.441 | −36.300 | 1.00 | 44.27 | E |
| ATOM | 16594 | CD | ARG | E | 271 | 86.096 | −16.839 | −35.034 | 1.00 | 44.27 | E |
| ATOM | 16595 | NE | ARG | E | 271 | 86.476 | −17.855 | −34.058 | 1.00 | 44.27 | E |
| ATOM | 16596 | CZ | ARG | E | 271 | 87.727 | −18.196 | −33.765 | 1.00 | 44.27 | E |
| ATOM | 16597 | NH1 | ARG | E | 271 | 88.744 | −17.606 | −34.377 | 1.00 | 44.27 | E |
| ATOM | 16598 | NH2 | ARG | E | 271 | 87.963 | −19.116 | −32.839 | 1.00 | 44.27 | E |
| ATOM | 16599 | C | ARG | E | 271 | 85.426 | −17.737 | −39.244 | 1.00 | 55.73 | E |
| ATOM | 16600 | O | ARG | E | 271 | 86.026 | −16.828 | −39.810 | 1.00 | 55.73 | E |
| ATOM | 16601 | N | TYR | E | 272 | 84.109 | −17.896 | −39.344 | 1.00 | 49.31 | E |
| ATOM | 16602 | CA | TYR | E | 272 | 83.303 | −16.979 | −40.142 | 1.00 | 49.31 | E |
| ATOM | 16603 | CB | TYR | E | 272 | 82.119 | −17.706 | −40.774 | 1.00 | 42.70 | E |
| ATOM | 16604 | CG | TYR | E | 272 | 81.274 | −16.800 | −41.657 | 1.00 | 40.17 | E |
| ATOM | 16605 | CD1 | TYR | E | 272 | 81.653 | −16.523 | −42.972 | 1.00 | 40.17 | E |
| ATOM | 16606 | CE1 | TYR | E | 272 | 80.898 | −15.666 | −43.771 | 1.00 | 40.17 | E |
| ATOM | 16607 | CD2 | TYR | E | 272 | 80.119 | −16.200 | −41.170 | 1.00 | 40.17 | E |
| ATOM | 16608 | CE2 | TYR | E | 272 | 79.361 | −15.353 | −41.967 | 1.00 | 40.17 | E |
| ATOM | 16609 | CZ | TYR | E | 272 | 79.754 | −15.089 | −43.259 | 1.00 | 40.17 | E |
| ATOM | 16610 | OH | TYR | E | 272 | 78.997 | −14.252 | −44.033 | 1.00 | 40.17 | E |
| ATOM | 16611 | C | TYR | E | 272 | 82.768 | −15.821 | −39.317 | 1.00 | 49.31 | E |
| ATOM | 16612 | O | TYR | E | 272 | 82.065 | −16.037 | −38.334 | 1.00 | 49.31 | E |
| ATOM | 16613 | N | HIS | E | 273 | 83.086 | −14.593 | −39.723 | 1.00 | 52.28 | E |
| ATOM | 16614 | CA | HIS | E | 273 | 82.614 | −13.421 | −38.996 | 1.00 | 52.28 | E |
| ATOM | 16615 | CB | HIS | E | 273 | 83.740 | −12.400 | −38.820 | 1.00 | 142.84 | E |
| ATOM | 16616 | CG | HIS | E | 273 | 84.744 | −12.797 | −37.785 | 1.00 | 123.57 | E |
| ATOM | 16617 | CD2 | HIS | E | 273 | 86.074 | −13.034 | −37.874 | 1.00 | 123.57 | E |
| ATOM | 16618 | ND1 | HIS | E | 273 | 84.400 | −13.036 | −36.471 | 1.00 | 123.57 | E |
| ATOM | 16619 | CE1 | HIS | E | 273 | 85.474 | −13.404 | −35.796 | 1.00 | 123.57 | E |
| ATOM | 16620 | NE2 | HIS | E | 273 | 86.504 | −13.411 | −36.624 | 1.00 | 123.57 | E |
| ATOM | 16621 | C | HIS | E | 273 | 81.412 | −12.766 | −39.642 | 1.00 | 52.28 | E |
| ATOM | 16622 | O | HIS | E | 273 | 81.454 | −12.311 | −40.785 | 1.00 | 52.28 | E |
| ATOM | 16623 | N | THR | E | 274 | 80.327 | −12.751 | −38.883 | 1.00 | 41.45 | E |
| ATOM | 16624 | CA | THR | E | 274 | 79.080 | −12.150 | −39.304 | 1.00 | 41.45 | E |
| ATOM | 16625 | CB | THR | E | 274 | 78.065 | −12.241 | −38.159 | 1.00 | 66.44 | E |
| ATOM | 16626 | OG1 | THR | E | 274 | 77.243 | −13.396 | −38.363 | 1.00 | 58.97 | E |
| ATOM | 16627 | CG2 | THR | E | 274 | 77.218 | −10.973 | −38.065 | 1.00 | 58.97 | E |
| ATOM | 16628 | C | THR | E | 274 | 79.257 | −10.695 | −39.740 | 1.00 | 41.45 | E |
| ATOM | 16629 | O | THR | E | 274 | 78.627 | −10.256 | −40.698 | 1.00 | 41.45 | E |
| ATOM | 16630 | N | GLY | E | 275 | 80.112 | −9.959 | −39.033 | 1.00 | 65.46 | E |
| ATOM | 16631 | CA | GLY | E | 275 | 80.357 | −8.570 | −39.368 | 1.00 | 65.46 | E |
| ATOM | 16632 | C | GLY | E | 275 | 81.026 | −8.380 | −40.718 | 1.00 | 65.46 | E |
| ATOM | 16633 | O | GLY | E | 275 | 80.437 | −7.800 | −41.633 | 1.00 | 65.46 | E |
| ATOM | 16634 | N | ASP | E | 276 | 82.250 | −8.882 | −40.849 | 1.00 | 61.37 | E |
| ATOM | 16635 | CA | ASP | E | 276 | 83.023 | −8.750 | −42.081 | 1.00 | 61.37 | E |
| ATOM | 16636 | CB | ASP | E | 276 | 84.469 | −9.160 | −41.827 | 1.00 | 138.64 | E |
| ATOM | 16637 | CG | ASP | E | 276 | 85.049 | −8.498 | −40.600 | 1.00 | 117.64 | E |
| ATOM | 16638 | OD1 | ASP | E | 276 | 85.266 | −7.264 | −40.631 | 1.00 | 117.64 | E |
| ATOM | 16639 | OD2 | ASP | E | 276 | 85.277 | −9.216 | −39.599 | 1.00 | 117.64 | E |
| ATOM | 16640 | C | ASP | E | 276 | 82.482 | −9.557 | −43.249 | 1.00 | 61.37 | E |
| ATOM | 16641 | O | ASP | E | 276 | 82.849 | −9.305 | −44.392 | 1.00 | 61.37 | E |
| ATOM | 16642 | N | LYS | E | 277 | 81.614 | −10.524 | −42.966 | 1.00 | 60.72 | E |
| ATOM | 16643 | CA | LYS | E | 277 | 81.039 | −11.371 | −44.008 | 1.00 | 60.72 | E |
| ATOM | 16644 | CB | LYS | E | 277 | 80.175 | −10.533 | −44.951 | 1.00 | 85.33 | E |
| ATOM | 16645 | CG | LYS | E | 277 | 78.921 | −9.982 | −44.297 | 1.00 | 70.54 | E |
| ATOM | 16646 | CD | LYS | E | 277 | 78.273 | −8.915 | −45.159 | 1.00 | 70.54 | E |
| ATOM | 16647 | CE | LYS | E | 277 | 76.981 | −8.393 | −44.547 | 1.00 | 70.54 | E |
| ATOM | 16648 | NZ | LYS | E | 277 | 75.847 | −9.362 | −44.685 | 1.00 | 70.54 | E |
| ATOM | 16649 | C | LYS | E | 277 | 82.143 | −12.092 | −44.790 | 1.00 | 60.72 | E |
| ATOM | 16650 | O | LYS | E | 277 | 82.161 | −12.095 | −46.025 | 1.00 | 60.72 | E |
| ATOM | 16651 | N | ARG | E | 278 | 83.060 | −12.704 | −44.050 | 1.00 | 51.98 | E |
| ATOM | 16652 | CA | ARG | E | 278 | 84.178 | −13.435 | −44.628 | 1.00 | 51.98 | E |
| ATOM | 16653 | CB | ARG | E | 278 | 85.212 | −12.451 | −45.159 | 1.00 | 97.59 | E |
| ATOM | 16654 | CG | ARG | E | 278 | 85.945 | −11.732 | −44.064 | 1.00 | 82.22 | E |
| ATOM | 16655 | CD | ARG | E | 278 | 86.782 | −10.598 | −44.600 | 1.00 | 82.22 | E |
| ATOM | 16656 | NE | ARG | E | 278 | 87.572 | −9.970 | −43.542 | 1.00 | 82.22 | E |
| ATOM | 16657 | CZ | ARG | E | 278 | 88.148 | −8.775 | −43.638 | 1.00 | 82.22 | E |
| ATOM | 16658 | NH1 | ARG | E | 278 | 88.028 | −8.060 | −44.752 | 1.00 | 82.22 | E |
| ATOM | 16659 | NH2 | ARG | E | 278 | 88.839 | −8.291 | −42.613 | 1.00 | 82.22 | E |
| ATOM | 16660 | C | ARG | E | 278 | 84.815 | −14.311 | −43.538 | 1.00 | 51.98 | E |
| ATOM | 16661 | O | ARG | E | 278 | 84.500 | −14.165 | −42.354 | 1.00 | 51.98 | E |
| ATOM | 16662 | N | PHE | E | 279 | 85.700 | −15.225 | −43.936 | 1.00 | 47.83 | E |
| ATOM | 16663 | CA | PHE | E | 279 | 86.379 | −16.092 | −42.974 | 1.00 | 47.83 | E |
| ATOM | 16664 | CB | PHE | E | 279 | 86.748 | −17.437 | −43.591 | 1.00 | 57.04 | E |
| ATOM | 16665 | CG | PHE | E | 279 | 85.609 | −18.405 | −43.662 | 1.00 | 40.81 | E |
| ATOM | 16666 | CD1 | PHE | E | 279 | 84.817 | −18.486 | −44.805 | 1.00 | 40.81 | E |
| ATOM | 16667 | CD2 | PHE | E | 279 | 85.321 | −19.243 | −42.581 | 1.00 | 40.81 | E |
| ATOM | 16668 | CE1 | PHE | E | 279 | 83.750 | −19.398 | −44.876 | 1.00 | 40.81 | E |
| ATOM | 16669 | CE2 | PHE | E | 279 | 84.264 | −20.147 | −42.640 | 1.00 | 40.81 | E |
| ATOM | 16670 | CZ | PHE | E | 279 | 83.477 | −20.229 | −43.788 | 1.00 | 40.81 | E |
| ATOM | 16671 | C | PHE | E | 279 | 87.648 | −15.447 | −42.473 | 1.00 | 47.83 | E |
| ATOM | 16672 | O | PHE | E | 279 | 88.261 | −14.658 | −43.170 | 1.00 | 47.83 | E |
| ATOM | 16673 | N | THR | E | 280 | 88.047 | −15.803 | −41.264 | 1.00 | 49.87 | E |
| ATOM | 16674 | CA | THR | E | 280 | 89.253 | −15.254 | −40.668 | 1.00 | 49.87 | E |
| ATOM | 16675 | CB | THR | E | 280 | 88.928 | −14.233 | −39.583 | 1.00 | 109.22 | E |
| ATOM | 16676 | OG1 | THR | E | 280 | 88.013 | −13.267 | −40.104 | 1.00 | 89.74 | E |
| ATOM | 16677 | CG2 | THR | E | 280 | 90.193 | −13.530 | −39.127 | 1.00 | 89.74 | E |
| ATOM | 16678 | C | THR | E | 280 | 90.048 | −16.359 | −40.019 | 1.00 | 49.87 | E |
| ATOM | 16679 | O | THR | E | 280 | 89.480 | −17.218 | −39.357 | 1.00 | 49.87 | E |
| ATOM | 16680 | N | LYS | E | 281 | 91.363 | −16.325 | −40.210 | 1.00 | 51.48 | E |
| ATOM | 16681 | CA | LYS | E | 281 | 92.267 | −17.320 | −39.637 | 1.00 | 51.48 | E |
| ATOM | 16682 | CB | LYS | E | 281 | 93.716 | −16.872 | −39.847 | 1.00 | 104.10 | E |
| ATOM | 16683 | CG | LYS | E | 281 | 93.906 | −15.368 | −39.710 | 1.00 | 80.04 | E |
| ATOM | 16684 | CD | LYS | E | 281 | 95.058 | −15.013 | −38.778 | 1.00 | 80.04 | E |
| ATOM | 16685 | CE | LYS | E | 281 | 96.412 | −15.434 | −39.335 | 1.00 | 80.04 | E |
| ATOM | 16686 | NZ | LYS | E | 281 | 97.540 | −15.003 | −38.450 | 1.00 | 80.04 | E |
| ATOM | 16687 | C | LYS | E | 281 | 92.012 | −17.552 | −38.145 | 1.00 | 51.48 | E |
| ATOM | 16688 | O | LYS | E | 281 | 91.717 | −16.619 | −37.397 | 1.00 | 51.48 | E |
| ATOM | 16689 | N | MET | E | 282 | 92.101 | −18.807 | −37.720 | 1.00 | 54.63 | E |
| ATOM | 16690 | CA | MET | E | 282 | 91.916 | −19.154 | −36.316 | 1.00 | 54.63 | E |
| ATOM | 16691 | CB | MET | E | 282 | 91.009 | −20.374 | −36.160 | 1.00 | 62.17 | E |
| ATOM | 16692 | CG | MET | E | 282 | 89.549 | −20.139 | −36.470 | 1.00 | 62.17 | E |
| ATOM | 16693 | SD | MET | E | 282 | 88.553 | −21.482 | −35.764 | 1.00 | 62.17 | E |
| ATOM | 16694 | CE | MET | E | 282 | 88.509 | −22.561 | −37.133 | 1.00 | 62.17 | E |
| ATOM | 16695 | C | MET | E | 282 | 93.276 | −19.487 | −35.711 | 1.00 | 54.63 | E |
| ATOM | 16696 | O | MET | E | 282 | 93.408 | −19.682 | −34.505 | 1.00 | 54.63 | E |
| ATOM | 16697 | N | THR | E | 283 | 94.282 | −19.560 | −36.570 | 1.00 | 97.74 | E |
| ATOM | 16698 | CA | THR | E | 283 | 95.639 | −19.877 | −36.159 | 1.00 | 97.74 | E |
| ATOM | 16699 | CB | THR | E | 283 | 95.981 | −21.328 | −36.516 | 1.00 | 86.74 | E |
| ATOM | 16700 | OG1 | THR | E | 283 | 95.145 | −22.206 | −35.761 | 1.00 | 86.74 | E |
| ATOM | 16701 | CG2 | THR | E | 283 | 97.424 | −21.636 | −36.204 | 1.00 | 86.74 | E |
| ATOM | 16702 | C | THR | E | 283 | 96.596 | −18.957 | −36.898 | 1.00 | 97.74 | E |
| ATOM | 16703 | O | THR | E | 283 | 96.242 | −18.400 | −37.938 | 1.00 | 97.74 | E |
| ATOM | 16704 | N | HIS | E | 284 | 97.801 | −18.791 | −36.357 | 1.00 | 90.45 | E |
| ATOM | 16705 | CA | HIS | E | 284 | 98.819 | −17.949 | −36.980 | 1.00 | 90.45 | E |
| ATOM | 16706 | CB | HIS | E | 284 | 100.103 | −17.985 | −36.150 | 1.00 | 142.16 | E |
| ATOM | 16707 | CG | HIS | E | 254 | 100.635 | −19.368 | −35.930 | 1.00 | 142.16 | E |
| ATOM | 16708 | CD2 | HIS | E | 284 | 101.777 | −19.963 | −36.348 | 1.00 | 142.16 | E |
| ATOM | 16709 | ND1 | HIS | E | 284 | 99.940 | −20.329 | −35.226 | 1.00 | 142.16 | E |
| ATOM | 16710 | CE1 | HIS | E | 284 | 100.630 | −21.456 | −35.222 | 1.00 | 142.16 | E |
| ATOM | 16711 | NE2 | HIS | E | 284 | 101.749 | −21.261 | −35.896 | 1.00 | 142.16 | E |
| ATOM | 16712 | C | HIS | E | 284 | 99.097 | −18.495 | −38.383 | 1.00 | 90.45 | E |
| ATOM | 16713 | O | HIS | E | 284 | 99.339 | −17.740 | −39.329 | 1.00 | 90.45 | E |
| ATOM | 16714 | N | LYS | E | 285 | 99.036 | −19.820 | −38.494 | 1.00 | 74.92 | E |
| ATOM | 16715 | CA | LYS | E | 285 | 99.281 | −20.546 | −39.735 | 1.00 | 74.92 | E |
| ATOM | 16716 | CB | LYS | E | 285 | 98.951 | −22.025 | −39.533 | 1.00 | 97.36 | E |
| ATOM | 16717 | CG | LYS | E | 285 | 100.048 | −22.975 | −39.963 | 1.00 | 97.36 | E |
| ATOM | 16718 | CD | LYS | E | 285 | 101.288 | −22.818 | −39.091 | 1.00 | 97.36 | E |
| ATOM | 16719 | CE | LYS | E | 285 | 102.390 | −23.791 | −39.507 | 1.00 | 97.36 | E |
| ATOM | 16720 | NZ | LYS | E | 285 | 103.608 | −23.666 | −38.655 | 1.00 | 97.36 | E |
| ATOM | 16721 | C | LYS | E | 285 | 98.508 | −20.031 | −40.947 | 1.00 | 74.92 | E |
| ATOM | 16722 | O | LYS | E | 285 | 98.987 | −20.129 | −42.078 | 1.00 | 74.92 | E |
| ATOM | 16723 | N | GLY | E | 286 | 97.314 | −19.494 | −40.715 | 1.00 | 80.66 | E |
| ATOM | 16724 | CA | GLY | E | 286 | 96.506 | −18.991 | −41.814 | 1.00 | 80.66 | E |
| ATOM | 16725 | C | GLY | E | 286 | 95.690 | −20.119 | −42.409 | 1.00 | 80.66 | E |
| ATOM | 16726 | O | GLY | E | 286 | 95.733 | −21.239 | −41.901 | 1.00 | 80.66 | E |
| ATOM | 16727 | N | PHE | E | 287 | 94.941 | −19.836 | −43.470 | 1.00 | 70.67 | E |
| ATOM | 16728 | CA | PHE | E | 287 | 94.131 | −20.866 | −44.114 | 1.00 | 70.67 | E |
| ATOM | 16729 | CB | PHE | E | 287 | 92.704 | −20.855 | −43.553 | 1.00 | 43.31 | E |
| ATOM | 16730 | CG | PHE | E | 287 | 91.902 | −19.652 | −43.942 | 1.00 | 43.31 | E |
| ATOM | 16731 | CD1 | PHE | E | 287 | 91.223 | −19.612 | −45.146 | 1.00 | 43.31 | E |
| ATOM | 16732 | CD2 | PHE | E | 287 | 91.831 | −18.558 | −43.101 | 1.00 | 43.31 | E |
| ATOM | 16733 | CE1 | PHE | E | 287 | 90.483 | −18.493 | −45.507 | 1.00 | 43.31 | E |
| ATOM | 16734 | CE2 | PHE | E | 287 | 91.089 | −17.428 | −43.453 | 1.00 | 43.31 | E |
| ATOM | 16735 | CZ | PHE | E | 287 | 90.416 | −17.394 | −44.654 | 1.00 | 43.31 | E |
| ATOM | 16736 | C | PHE | E | 287 | 94.095 | −20.681 | −45.626 | 1.00 | 70.67 | E |
| ATOM | 16737 | O | PHE | E | 287 | 94.023 | −19.558 | −46.121 | 1.00 | 70.67 | E |
| ATOM | 16738 | N | PRO | E | 288 | 94.138 | −21.788 | −46.380 | 1.00 | 66.14 | E |
| ATOM | 16739 | CD | PRO | E | 288 | 94.130 | −23.175 | −45.893 | 1.00 | 55.94 | E |
| ATOM | 16740 | CA | PRO | E | 288 | 94.114 | −21.747 | −47.846 | 1.00 | 66.14 | E |
| ATOM | 16741 | CB | PRO | E | 288 | 94.343 | −23.202 | −48.231 | 1.00 | 55.94 | E |
| ATOM | 16742 | CG | PRO | E | 288 | 93.674 | −23.941 | −47.113 | 1.00 | 55.94 | E |
| ATOM | 16743 | C | PRO | E | 288 | 92.807 | −21.195 | −48.403 | 1.00 | 66.14 | E |
| ATOM | 16744 | O | PRO | E | 288 | 91.724 | −21.701 | −48.103 | 1.00 | 66.14 | E |
| ATOM | 16745 | N | LYS | E | 289 | 92.905 | −20.148 | −49.211 | 1.00 | 67.89 | E |
| ATOM | 16746 | CA | LYS | E | 289 | 91.714 | −19.550 | −49.793 | 1.00 | 67.89 | E |
| ATOM | 16747 | CB | LYS | E | 289 | 91.982 | −18.089 | −50.156 | 1.00 | 107.37 | E |
| ATOM | 16748 | CG | LYS | E | 289 | 91.965 | −17.172 | −48.937 | 1.00 | 107.37 | E |
| ATOM | 16749 | CD | LYS | E | 289 | 92.253 | −15.728 | −49.296 | 1.00 | 107.37 | E |
| ATOM | 16750 | CE | LYS | E | 289 | 93.713 | −15.537 | −49.701 | 1.00 | 107.37 | E |
| ATOM | 16751 | NZ | LYS | E | 289 | 94.039 | −14.116 | −50.035 | 1.00 | 107.37 | E |
| ATOM | 16752 | C | LYS | E | 289 | 91.246 | −20.350 | −51.001 | 1.00 | 67.89 | E |
| ATOM | 16753 | O | LYS | E | 289 | 91.488 | −19.984 | −52.152 | 1.00 | 67.89 | E |
| ATOM | 16754 | N | VAL | E | 290 | 90.562 | −21.452 | −50.706 | 1.00 | 67.79 | E |
| ATOM | 16755 | CA | VAL | E | 290 | 90.039 | −22.353 | −51.723 | 1.00 | 67.79 | E |
| ATOM | 16756 | CB | VAL | E | 290 | 90.929 | −23.604 | −51.854 | 1.00 | 59.05 | E |
| ATOM | 16757 | CG1 | VAL | E | 290 | 92.376 | −23.189 | −52.049 | 1.00 | 59.05 | E |
| ATOM | 16758 | CG2 | VAL | E | 290 | 90.806 | −24.471 | −50.605 | 1.00 | 59.05 | E |
| ATOM | 16759 | C | VAL | E | 290 | 88.642 | −22.801 | −51.325 | 1.00 | 67.79 | E |
| ATOM | 16760 | O | VAL | E | 290 | 88.212 | −22.592 | −50.188 | 1.00 | 67.79 | E |
| ATOM | 16761 | N | GLY | E | 291 | 87.943 | −23.422 | −52.269 | 1.00 | 61.07 | E |
| ATOM | 16762 | CA | GLY | E | 291 | 86.601 | −23.912 | −52.008 | 1.00 | 61.07 | E |
| ATOM | 16763 | C | GLY | E | 291 | 85.645 | −22.936 | −51.343 | 1.00 | 61.07 | E |
| ATOM | 16764 | O | GLY | E | 291 | 85.512 | −21.785 | −51.757 | 1.00 | 61.07 | E |
| ATOM | 16765 | N | MET | E | 292 | 84.973 | −23.410 | −50.301 | 1.00 | 63.42 | E |
| ATOM | 16766 | CA | MET | E | 292 | 84.004 | −22.608 | −49.568 | 1.00 | 63.42 | E |
| ATOM | 16767 | CB | MET | E | 292 | 83.217 | −23.498 | −48.624 | 1.00 | 60.15 | E |
| ATOM | 16768 | CG | MET | E | 292 | 84.091 | −24.013 | −47.500 | 1.00 | 60.15 | E |
| ATOM | 16769 | SD | MET | E | 292 | 83.153 | −24.619 | −46.116 | 1.00 | 60.15 | E |
| ATOM | 16770 | CE | MET | E | 292 | 83.578 | −26.328 | −46.151 | 1.00 | 60.15 | E |
| ATOM | 16771 | C | MET | E | 292 | 84.672 | −21.526 | −48.734 | 1.00 | 63.42 | E |
| ATOM | 16772 | O | MET | E | 292 | 83.998 | −20.768 | −48.043 | 1.00 | 63.42 | E |
| ATOM | 16773 | N | LEU | E | 293 | 85.992 | −21.456 | −48.784 | 1.00 | 63.20 | E |
| ATOM | 16774 | CA | LEU | E | 293 | 86.704 | −20.476 | −47.987 | 1.00 | 63.20 | E |
| ATOM | 16775 | CB | LEU | E | 293 | 87.972 | −21.107 | −47.414 | 1.00 | 51.99 | E |
| ATOM | 16776 | CG | LEU | E | 293 | 87.732 | −22.260 | −46.431 | 1.00 | 51.99 | E |
| ATOM | 16777 | CD1 | LEU | E | 293 | 89.050 | −22.913 | −46.043 | 1.00 | 51.99 | E |
| ATOM | 16778 | CD2 | LEU | E | 293 | 87.016 | −21.725 | −45.206 | 1.00 | 51.99 | E |
| ATOM | 16779 | C | LEU | E | 293 | 87.064 | −19.150 | −48.653 | 1.00 | 63.20 | E |
| ATOM | 16780 | O | LEU | E | 293 | 87.624 | −18.281 | −47.988 | 1.00 | 63.20 | E |
| ATOM | 16781 | N | THR | E | 294 | 86.766 | −18.978 | −49.942 | 1.00 | 60.94 | E |
| ATOM | 16782 | CA | THR | E | 294 | 87.088 | −17.717 | −50.609 | 1.00 | 60.94 | E |
| ATOM | 16783 | CB | THR | E | 294 | 88.144 | −17.868 | −51.696 | 1.00 | 87.36 | E |
| ATOM | 16784 | OG1 | THR | E | 294 | 89.156 | −18.770 | −51.253 | 1.00 | 87.36 | E |
| ATOM | 16785 | CG2 | THR | E | 294 | 88.781 | −16.509 | −51.990 | 1.00 | 87.36 | E |
| ATOM | 16786 | C | THR | E | 294 | 85.898 | −17.098 | −51.284 | 1.00 | 60.94 | E |
| ATOM | 16787 | O | THR | E | 294 | 84.993 | −17.798 | −51.722 | 1.00 | 60.94 | E |
| ATOM | 16788 | N | VAL | E | 295 | 85.915 | −15.775 | −51.383 | 1.00 | 61.47 | E |
| ATOM | 16789 | CA | VAL | E | 295 | 84.833 | −15.056 | −52.027 | 1.00 | 61.47 | E |
| ATOM | 16790 | CB | VAL | E | 295 | 84.979 | −13.555 | −51.809 | 1.00 | 88.28 | E |
| ATOM | 16791 | CG1 | VAL | E | 295 | 83.796 | −12.819 | −52.433 | 1.00 | 88.28 | E |
| ATOM | 16792 | CG2 | VAL | E | 295 | 85.085 | −13.271 | −50.318 | 1.00 | 88.28 | E |
| ATOM | 16793 | C | VAL | E | 295 | 84.905 | −15.365 | −53.512 | 1.00 | 61.47 | E |
| ATOM | 16794 | O | VAL | E | 295 | 83.916 | −15.218 | −54.236 | 1.00 | 61.47 | E |
| ATOM | 16795 | N | LYS | E | 296 | 86.082 | −15.816 | −53.947 | 1.00 | 63.62 | E |
| ATOM | 16796 | CA | LYS | E | 296 | 86.330 | −16.159 | −55.348 | 1.00 | 63.62 | E |
| ATOM | 16797 | CB | LYS | E | 296 | 87.774 | −16.645 | −55.528 | 1.00 | 114.17 | E |
| ATOM | 16798 | CG | LYS | E | 296 | 88.843 | −15.636 | −55.143 | 1.00 | 114.17 | E |
| ATOM | 16799 | CD | LYS | E | 296 | 88.969 | −14.525 | −56.170 | 1.00 | 114.17 | E |
| ATOM | 16800 | CE | LYS | E | 296 | 89.538 | −15.049 | −57.484 | 1.00 | 114.17 | E |
| ATOM | 16801 | NZ | LYS | E | 296 | 89.662 | −13.984 | −58.522 | 1.00 | 114.17 | E |
| ATOM | 16802 | C | LYS | E | 296 | 85.382 | −17.253 | −55.824 | 1.00 | 63.62 | E |
| ATOM | 16803 | O | LYS | E | 296 | 85.007 | −17.284 | −56.996 | 1.00 | 63.62 | E |
| ATOM | 16804 | N | ASN | E | 297 | 84.995 | −18.140 | −54.907 | 1.00 | 50.03 | E |
| ATOM | 16805 | CA | ASN | E | 297 | 84.120 | −19.266 | −55.233 | 1.00 | 50.03 | E |
| ATOM | 16806 | CB | ASN | E | 297 | 83.951 | −20.186 | −54.023 | 1.00 | 51.76 | E |
| ATOM | 16807 | CG | ASN | E | 297 | 83.283 | −21.492 | −54.381 | 1.00 | 51.76 | E |
| ATOM | 16808 | OD1 | ASN | E | 297 | 82.141 | −21.521 | −54.846 | 1.00 | 51.76 | E |
| ATOM | 16809 | ND2 | ASN | E | 297 | 83.994 | −22.585 | −54.171 | 1.00 | 51.76 | E |
| ATOM | 16810 | C | ASN | E | 297 | 82.754 | −18.857 | −55.746 | 1.00 | 50.03 | E |
| ATOM | 16811 | O | ASN | E | 297 | 82.102 | −17.963 | −55.208 | 1.00 | 50.03 | E |
| ATOM | 16812 | N | THR | E | 298 | 82.327 | −19.545 | −56.796 | 1.00 | 52.54 | E |
| ATOM | 16813 | CA | THR | E | 298 | 81.046 | −19.275 | −57.426 | 1.00 | 52.54 | E |
| ATOM | 16814 | CB | THR | E | 298 | 80.880 | −20.166 | −58.676 | 1.00 | 75.13 | E |
| ATOM | 16815 | OG1 | THR | E | 298 | 79.498 | −20.238 | −59.036 | 1.00 | 75.13 | E |
| ATOM | 16816 | CG2 | THR | E | 298 | 81.427 | −21.560 | −58.414 | 1.00 | 75.13 | E |
| ATOM | 16817 | C | THR | E | 298 | 79.857 | −19.450 | −56.484 | 1.00 | 52.54 | E |
| ATOM | 16818 | O | THR | E | 298 | 78.823 | −18.827 | −56.675 | 1.00 | 52.54 | E |
| ATOM | 16819 | N | LEU | E | 299 | 80.011 | −20.282 | −55.461 | 1.00 | 55.13 | E |
| ATOM | 16820 | CA | LEU | E | 299 | 78.927 | −20.528 | −54.514 | 1.00 | 55.13 | E |
| ATOM | 16821 | CB | LEU | E | 299 | 78.866 | −22.010 | −54.167 | 1.00 | 49.87 | E |
| ATOM | 16822 | CG | LEU | E | 299 | 78.472 | −22.919 | −55.329 | 1.00 | 49.87 | E |
| ATOM | 16823 | CD1 | LEU | E | 299 | 78.583 | −24.360 | −54.885 | 1.00 | 49.87 | E |
| ATOM | 16824 | CD2 | LEU | E | 299 | 77.059 | −22.592 | −55.788 | 1.00 | 49.87 | E |
| ATOM | 16825 | C | LEU | E | 299 | 79.014 | −19.712 | −53.226 | 1.00 | 55.13 | E |
| ATOM | 16826 | O | LEU | E | 299 | 78.226 | −19.907 | −52.298 | 1.00 | 55.13 | E |
| ATOM | 16827 | N | TRP | E | 300 | 79.969 | −18.795 | −53.171 | 1.00 | 46.39 | E |
| ATOM | 16828 | CA | TRP | E | 300 | 80.136 | −17.951 | −52.002 | 1.00 | 46.39 | E |
| ATOM | 16829 | CB | TRP | E | 300 | 81.122 | −16.833 | −52.309 | 1.00 | 54.71 | E |
| ATOM | 16830 | CG | TRP | E | 300 | 81.376 | −15.954 | −51.137 | 1.00 | 54.71 | E |
| ATOM | 16831 | CD2 | TRP | E | 300 | 81.914 | −16.355 | −49.876 | 1.00 | 54.71 | E |
| ATOM | 16832 | CE2 | TRP | E | 300 | 81.980 | −15.204 | −49.064 | 1.00 | 54.71 | E |
| ATOM | 16833 | CE3 | TRP | E | 300 | 82.351 | −17.578 | −49.354 | 1.00 | 54.71 | E |
| ATOM | 16834 | CD1 | TRP | E | 300 | 81.143 | −14.615 | −51.046 | 1.00 | 54.71 | E |
| ATOM | 16835 | NE1 | TRP | E | 300 | 81.506 | −14.155 | −49.803 | 1.00 | 54.71 | E |
| ATOM | 16836 | CZ2 | TRP | E | 300 | 82.462 | −15.238 | −47.755 | 1.00 | 54.71 | E |
| ATOM | 16837 | CZ3 | TRP | E | 300 | 82.831 | −17.611 | −48.048 | 1.00 | 54.71 | E |
| ATOM | 16838 | CH2 | TRP | E | 300 | 82.881 | −16.446 | −47.266 | 1.00 | 54.71 | E |
| ATOM | 16839 | C | TRP | E | 300 | 78.821 | −17.342 | −51.510 | 1.00 | 46.39 | E |
| ATOM | 16840 | O | TRP | E | 300 | 78.556 | −17.332 | −50.309 | 1.00 | 46.39 | E |
| ATOM | 16841 | N | LYS | E | 301 | 78.009 | −16.828 | −52.430 | 1.00 | 44.38 | E |
| ATOM | 16842 | CA | LYS | E | 301 | 76.728 | −16.228 | −52.067 | 1.00 | 44.38 | E |
| ATOM | 16843 | CB | LYS | E | 301 | 75.955 | −15.789 | −53.322 | 1.00 | 83.98 | E |
| ATOM | 16844 | CG | LYS | E | 301 | 76.699 | −14.774 | −54.188 | 1.00 | 83.98 | E |
| ATOM | 16845 | CD | LYS | E | 301 | 77.244 | −13.614 | −53.351 | 1.00 | 83.98 | E |
| ATOM | 16846 | CE | LYS | E | 301 | 78.330 | −12.835 | −54.095 | 1.00 | 83.98 | E |
| ATOM | 16847 | NZ | LYS | E | 301 | 79.158 | −12.001 | −53.175 | 1.00 | 83.98 | E |
| ATOM | 16848 | C | LYS | E | 301 | 75.878 | −17.202 | −51.242 | 1.00 | 44.38 | E |
| ATOM | 16849 | O | LYS | E | 301 | 75.186 | −16.793 | −50.319 | 1.00 | 44.38 | E |
| ATOM | 16850 | N | ASP | E | 302 | 75.926 | −18.492 | −51.566 | 1.00 | 48.27 | E |
| ATOM | 16851 | CA | ASP | E | 302 | 75.149 | −19.465 | −50.813 | 1.00 | 48.27 | E |
| ATOM | 16852 | CB | ASP | E | 302 | 75.063 | −20.790 | −51.574 | 1.00 | 98.36 | E |
| ATOM | 16853 | CG | ASP | E | 302 | 74.164 | −20.698 | −52.793 | 1.00 | 98.36 | E |
| ATOM | 16854 | OD1 | ASP | E | 302 | 73.025 | −20.208 | −52.643 | 1.00 | 98.36 | E |
| ATOM | 16855 | OD2 | ASP | E | 302 | 74.586 | −21.118 | −53.894 | 1.00 | 98.36 | E |
| ATOM | 16856 | C | ASP | E | 302 | 75.775 | −19.674 | −49.440 | 1.00 | 48.27 | E |
| ATOM | 16857 | O | ASP | E | 302 | 75.074 | −19.860 | −48.451 | 1.00 | 48.27 | E |
| ATOM | 16858 | N | VAL | E | 303 | 77.100 | −19.634 | −49.376 | 1.00 | 32.68 | E |
| ATOM | 16859 | CA | VAL | E | 303 | 77.782 | −19.804 | −48.103 | 1.00 | 32.68 | E |
| ATOM | 16860 | CB | VAL | E | 303 | 79.310 | −19.734 | −48.270 | 1.00 | 31.85 | E |
| ATOM | 16861 | CG1 | VAL | E | 303 | 79.998 | −19.777 | −46.907 | 1.00 | 21.16 | E |
| ATOM | 16862 | CG2 | VAL | E | 303 | 79.778 | −20.897 | −49.099 | 1.00 | 21.16 | E |
| ATOM | 16863 | C | VAL | E | 303 | 77.334 | −18.690 | −47.160 | 1.00 | 32.68 | E |
| ATOM | 16864 | O | VAL | E | 303 | 76.896 | −18.949 | −46.033 | 1.00 | 32.68 | E |
| ATOM | 16865 | N | LYS | E | 304 | 77.444 | −17.449 | −47.628 | 1.00 | 51.77 | E |
| ATOM | 16866 | CA | LYS | E | 304 | 77.027 | −16.279 | −46.843 | 1.00 | 51.77 | E |
| ATOM | 16867 | CB | LYS | E | 304 | 77.058 | −15.012 | −47.700 | 1.00 | 89.51 | E |
| ATOM | 16868 | CG | LYS | E | 304 | 78.402 | −14.333 | −47.831 | 1.00 | 71.43 | E |
| ATOM | 16869 | CD | LYS | E | 304 | 78.264 | −13.121 | −48.735 | 1.00 | 71.43 | E |
| ATOM | 16870 | CE | LYS | E | 304 | 79.558 | −12.335 | −48.818 | 1.00 | 71.43 | E |
| ATOM | 16871 | NZ | LYS | E | 304 | 79.473 | −11.230 | −49.824 | 1.00 | 71.43 | E |
| ATOM | 16872 | C | LYS | E | 304 | 75.605 | −16.457 | −46.323 | 1.00 | 51.77 | E |
| ATOM | 16873 | O | LYS | E | 304 | 75.316 | −16.204 | −45.159 | 1.00 | 51.77 | E |
| ATOM | 16874 | N | ALA | E | 305 | 74.719 | −16.901 | −47.202 | 1.00 | 43.85 | E |
| ATOM | 16875 | CA | ALA | E | 305 | 73.328 | −17.089 | −46.844 | 1.00 | 43.85 | E |
| ATOM | 16876 | CB | ALA | E | 305 | 72.502 | −17.282 | −48.103 | 1.00 | 26.45 | E |
| ATOM | 16877 | C | ALA | E | 305 | 73.075 | −18.233 | −45.877 | 1.00 | 43.85 | E |
| ATOM | 16878 | O | ALA | E | 305 | 72.411 | −18.043 | −44.856 | 1.00 | 43.85 | E |
| ATOM | 16879 | N | TYR | E | 306 | 73.593 | −19.419 | −46.193 | 1.00 | 45.84 | E |
| ATOM | 16880 | CA | TYR | E | 306 | 73.369 | −20.577 | −45.344 | 1.00 | 45.84 | E |
| ATOM | 16881 | CB | TYR | E | 306 | 73.734 | −21.853 | −46.087 | 1.00 | 53.48 | E |
| ATOM | 16882 | CG | TYR | E | 306 | 72.820 | −22.149 | −47.248 | 1.00 | 48.17 | E |
| ATOM | 16883 | CD1 | TYR | E | 306 | 73.287 | −22.847 | −48.367 | 1.00 | 48.17 | E |
| ATOM | 16884 | CE1 | TYR | E | 306 | 72.451 | −23.131 | −49.442 | 1.00 | 48.17 | E |
| ATOM | 16885 | CD2 | TYR | E | 306 | 71.487 | −21.745 | −47.232 | 1.00 | 48.17 | E |
| ATOM | 16886 | CE2 | TYR | E | 306 | 70.644 | −22.027 | −48.299 | 1.00 | 48.17 | E |
| ATOM | 16887 | CZ | TYR | E | 306 | 71.128 | −22.718 | −49.398 | 1.00 | 48.17 | E |
| ATOM | 16888 | OH | TYR | E | 306 | 70.286 | −23.003 | −50.448 | 1.00 | 48.17 | E |
| ATOM | 16889 | C | TYR | E | 306 | 74.121 | −20.521 | −44.036 | 1.00 | 45.84 | E |
| ATOM | 16890 | O | TYR | E | 306 | 73.532 | −20.694 | −42.980 | 1.00 | 45.84 | E |
| ATOM | 16891 | N | LEU | E | 307 | 75.419 | −20.274 | −44.102 | 1.00 | 36.75 | E |
| ATOM | 16892 | CA | LEU | E | 307 | 76.232 | −20.235 | −42.900 | 1.00 | 36.75 | E |
| ATOM | 16893 | CB | LEU | E | 307 | 77.687 | −20.513 | −43.247 | 1.00 | 66.34 | E |
| ATOM | 16894 | CG | LEU | E | 307 | 78.683 | −20.315 | −42.106 | 1.00 | 34.47 | E |
| ATOM | 16895 | CD1 | LEU | E | 307 | 78.350 | −21.224 | −40.942 | 1.00 | 34.47 | E |
| ATOM | 16896 | CD2 | LEU | E | 307 | 80.090 | −20.587 | −42.629 | 1.00 | 34.47 | E |
| ATOM | 16897 | C | LEU | E | 307 | 76.121 | −18.903 | −42.181 | 1.00 | 36.75 | E |
| ATOM | 16898 | O | LEU | E | 307 | 75.883 | −18.857 | −40.971 | 1.00 | 36.75 | E |
| ATOM | 16899 | N | GLY | E | 308 | 76.305 | −17.817 | −42.928 | 1.00 | 44.51 | E |
| ATOM | 16900 | CA | GLY | E | 308 | 76.213 | −16.499 | −42.334 | 1.00 | 44.51 | E |
| ATOM | 16901 | C | GLY | E | 308 | 74.840 | −16.254 | −41.733 | 1.00 | 44.51 | E |
| ATOM | 16902 | O | GLY | E | 308 | 74.725 | −15.810 | −40.595 | 1.00 | 44.51 | E |
| ATOM | 16903 | N | GLY | E | 309 | 73.791 | −16.554 | −42.489 | 1.00 | 42.78 | E |
| ATOM | 16904 | CA | GLY | E | 309 | 72.448 | −16.324 | −41.990 | 1.00 | 42.78 | E |
| ATOM | 16905 | C | GLY | E | 309 | 72.143 | −17.064 | −40.708 | 1.00 | 42.78 | E |
| ATOM | 16906 | O | GLY | E | 309 | 71.614 | −16.495 | −39.754 | 1.00 | 42.78 | E |
| ATOM | 16907 | N | PHE | E | 310 | 72.474 | −18.348 | −40.692 | 1.00 | 34.86 | E |
| ATOM | 16908 | CA | PHE | E | 310 | 72.227 | −19.185 | −39.528 | 1.00 | 34.86 | E |
| ATOM | 16909 | CB | PHE | E | 310 | 72.730 | −20.593 | −39.803 | 1.00 | 37.18 | E |
| ATOM | 16910 | CG | PHE | E | 310 | 72.307 | −21.597 | −38.785 | 1.00 | 33.01 | E |
| ATOM | 16911 | CD1 | PHE | E | 310 | 70.960 | −21.859 | −38.568 | 1.00 | 33.01 | E |
| ATOM | 16912 | CD2 | PHE | E | 310 | 73.256 | −22.299 | −38.055 | 1.00 | 33.01 | E |
| ATOM | 16913 | CE1 | PHE | E | 310 | 70.561 | −22.806 | −37.639 | 1.00 | 33.01 | E |
| ATOM | 16914 | CE2 | PHE | E | 310 | 72.872 | −23.253 | −37.121 | 1.00 | 33.01 | E |
| ATOM | 16915 | CZ | PHE | E | 310 | 71.518 | −23.506 | −36.914 | 1.00 | 33.01 | E |
| ATOM | 16916 | C | PHE | E | 310 | 72.940 | −18.620 | −38.311 | 1.00 | 34.86 | E |
| ATOM | 16917 | O | PHE | E | 310 | 72.373 | −18.547 | −37.224 | 1.00 | 34.86 | E |
| ATOM | 16918 | N | GLU | E | 311 | 74.190 | −18.220 | −38.513 | 1.00 | 37.57 | E |
| ATOM | 16919 | CA | GLU | E | 311 | 75.010 | −17.666 | −37.452 | 1.00 | 37.57 | E |
| ATOM | 16920 | CB | GLU | E | 311 | 76.464 | −17.597 | −37.899 | 1.00 | 76.77 | E |
| ATOM | 16921 | CG | GLU | E | 311 | 77.433 | −17.563 | −36.749 | 1.00 | 61.00 | E |
| ATOM | 16922 | CD | GLU | E | 311 | 78.859 | −17.308 | −37.185 | 1.00 | 61.00 | E |
| ATOM | 16923 | OE1 | GLU | E | 311 | 79.279 | −17.837 | −38.232 | 1.00 | 61.00 | E |
| ATOM | 16924 | OE2 | GLU | E | 311 | 79.573 | −16.586 | −36.467 | 1.00 | 61.00 | E |
| ATOM | 16925 | C | GLU | E | 311 | 74.544 | −16.271 | −37.055 | 1.00 | 37.57 | E |
| ATOM | 16926 | O | GLU | E | 311 | 74.630 | −15.894 | −35.898 | 1.00 | 37.57 | E |
| ATOM | 16927 | N | TYR | E | 312 | 74.041 | −15.501 | −38.011 | 1.00 | 44.25 | E |
| ATOM | 16928 | CA | TYR | E | 312 | 73.591 | −14.156 | −37.707 | 1.00 | 44.25 | E |
| ATOM | 16929 | CB | TYR | E | 312 | 73.340 | −13.365 | −38.986 | 1.00 | 54.28 | E |
| ATOM | 16930 | CG | TYR | E | 312 | 72.939 | −11.940 | −38.711 | 1.00 | 48.98 | E |
| ATOM | 16931 | CD1 | TYR | E | 312 | 73.873 | −11.016 | −38.253 | 1.00 | 48.98 | E |
| ATOM | 16932 | CE1 | TYR | E | 312 | 73.512 | −9.701 | −37.957 | 1.00 | 48.98 | E |
| ATOM | 16933 | CD2 | TYR | E | 312 | 71.620 | −11.521 | −38.868 | 1.00 | 48.98 | E |
| ATOM | 16934 | CE2 | TYR | E | 312 | 71.241 | −10.207 | −38.571 | 1.00 | 48.98 | E |
| ATOM | 16935 | CZ | TYR | E | 312 | 72.193 | −9.300 | −38.115 | 1.00 | 48.98 | E |
| ATOM | 16936 | OH | TYR | E | 312 | 71.826 | −8.001 | −37.804 | 1.00 | 48.98 | E |
| ATOM | 16937 | C | TYR | E | 312 | 72.334 | −14.109 | −36.857 | 1.00 | 44.25 | E |
| ATOM | 16938 | O | TYR | E | 312 | 72.192 | −13.230 | −36.015 | 1.00 | 44.25 | E |
| ATOM | 16939 | N | MET | E | 313 | 71.413 | −15.037 | −37.074 | 1.00 | 46.56 | E |
| ATOM | 16940 | CA | MET | E | 313 | 70.176 | −15.013 | −36.314 | 1.00 | 46.56 | E |
| ATOM | 16941 | CB | MET | E | 313 | 69.281 | −13.880 | −36.823 | 1.00 | 72.91 | E |
| ATOM | 16942 | CG | MET | E | 313 | 67.990 | −13.738 | −36.059 | 1.00 | 72.76 | E |
| ATOM | 16943 | SD | MET | E | 313 | 66.955 | −12.487 | −36.761 | 1.00 | 72.76 | E |
| ATOM | 16944 | CE | MET | E | 313 | 68.056 | −11.048 | −36.678 | 1.00 | 72.76 | E |
| ATOM | 16945 | C | MET | E | 313 | 69.396 | −16.319 | −36.372 | 1.00 | 46.56 | E |
| ATOM | 16946 | O | MET | E | 313 | 68.742 | −16.712 | −35.398 | 1.00 | 46.56 | E |
| ATOM | 16947 | N | GLY | E | 314 | 69.462 | −16.987 | −37.518 | 1.00 | 31.22 | E |
| ATOM | 16948 | CA | GLY | E | 314 | 68.733 | −18.226 | −37.687 | 1.00 | 31.22 | E |
| ATOM | 16949 | C | GLY | E | 314 | 68.817 | −19.168 | −36.522 | 1.00 | 31.22 | E |
| ATOM | 16950 | O | GLY | E | 314 | 67.800 | −19.597 | −35.984 | 1.00 | 31.22 | E |
| ATOM | 16951 | N | CYS | E | 315 | 70.040 | −19.490 | −36.133 | 1.00 | 39.90 | E |
| ATOM | 16952 | CA | CYS | E | 315 | 70.259 | −20.411 | −35.037 | 1.00 | 39.90 | E |
| ATOM | 16953 | CB | CYS | E | 315 | 71.746 | −20.621 | −34.834 | 1.00 | 57.75 | E |
| ATOM | 16954 | SG | CYS | E | 315 | 72.098 | −22.014 | −33.782 | 1.00 | 63.12 | E |
| ATOM | 16955 | C | CYS | E | 315 | 69.621 | −19.958 | −33.727 | 1.00 | 39.90 | E |
| ATOM | 16956 | O | CYS | E | 315 | 68.870 | −20.712 | −33.101 | 1.00 | 39.90 | E |
| ATOM | 16957 | N | SER | E | 316 | 69.910 | −18.729 | −33.313 | 1.00 | 48.44 | E |
| ATOM | 16958 | CA | SER | E | 316 | 69.345 | −18.215 | −32.070 | 1.00 | 48.44 | E |
| ATOM | 16959 | CB | SER | E | 316 | 69.772 | −16.770 | −31.851 | 1.00 | 56.71 | E |
| ATOM | 16960 | OG | SER | E | 316 | 69.162 | −15.928 | −32.803 | 1.00 | 62.21 | E |
| ATOM | 16961 | C | SER | E | 316 | 67.822 | −18.305 | −32.038 | 1.00 | 48.44 | E |
| ATOM | 16962 | O | SER | E | 316 | 67.236 | −18.404 | −30.967 | 1.00 | 48.44 | E |
| ATOM | 16963 | N | LEU | E | 317 | 67.178 | −18.255 | −33.198 | 1.00 | 41.77 | E |
| ATOM | 16964 | CA | LEU | E | 317 | 65.726 | −18.368 | −33.240 | 1.00 | 41.77 | E |
| ATOM | 16965 | CB | LEU | E | 317 | 65.174 | −17.748 | −34.527 | 1.00 | 54.64 | E |
| ATOM | 16966 | CG | LEU | E | 317 | 65.244 | −16.222 | −34.625 | 1.00 | 47.15 | E |
| ATOM | 16967 | CD1 | LEU | E | 317 | 64.821 | −15.755 | −36.005 | 1.00 | 47.15 | E |
| ATOM | 16968 | CD2 | LEU | E | 317 | 64.347 | −15.617 | −33.556 | 1.00 | 47.15 | E |
| ATOM | 16969 | C | LEU | E | 317 | 65.311 | −19.847 | −33.166 | 1.00 | 41.77 | E |
| ATOM | 16970 | O | LEU | E | 317 | 64.368 | −20.228 | −32.462 | 1.00 | 41.77 | E |
| ATOM | 16971 | N | ALA | E | 318 | 66.031 | −20.684 | −33.892 | 1.00 | 42.09 | E |
| ATOM | 16972 | CA | ALA | E | 318 | 65.732 | −22.096 | −33.920 | 1.00 | 42.09 | E |
| ATOM | 16973 | CB | ALA | E | 318 | 66.619 | −22.779 | −34.917 | 1.00 | 38.05 | E |
| ATOM | 16974 | C | ALA | E | 318 | 65.896 | −22.754 | −32.566 | 1.00 | 42.09 | E |
| ATOM | 16975 | O | ALA | E | 318 | 65.021 | −23.505 | −32.119 | 1.00 | 42.09 | E |
| ATOM | 16976 | N | ILE | E | 319 | 67.015 | −22.479 | −31.902 | 1.00 | 45.42 | E |
| ATOM | 16977 | CA | ILE | E | 319 | 67.277 | −23.098 | −30.614 | 1.00 | 45.42 | E |
| ATOM | 16978 | CB | ILE | E | 319 | 68.498 | −22.471 | −29.929 | 1.00 | 49.85 | E |
| ATOM | 16979 | CG2 | ILE | E | 319 | 68.153 | −21.068 | −29.435 | 1.00 | 24.41 | E |
| ATOM | 16980 | CG1 | ILE | E | 319 | 68.945 | −23.369 | −28.768 | 1.00 | 24.41 | E |
| ATOM | 16981 | CD1 | ILE | E | 319 | 70.232 | −22.936 | −28.069 | 1.00 | 24.41 | E |
| ATOM | 16982 | C | ILE | E | 319 | 66.074 | −23.024 | −29.677 | 1.00 | 45.42 | E |
| ATOM | 16983 | O | ILE | E | 319 | 65.879 | −23.926 | −28.862 | 1.00 | 45.42 | E |
| ATOM | 16984 | N | LEU | E | 320 | 65.264 | −21.970 | −29.803 | 1.00 | 51.29 | E |
| ATOM | 16985 | CA | LEU | E | 320 | 64.092 | −21.818 | −28.952 | 1.00 | 51.29 | E |
| ATOM | 16986 | CB | LEU | E | 320 | 63.554 | −20.386 | −29.029 | 1.00 | 75.94 | E |
| ATOM | 16987 | CG | LEU | E | 320 | 64.490 | −19.303 | −28.470 | 1.00 | 53.78 | E |
| ATOM | 16988 | CD1 | LEU | E | 320 | 63.817 | −17.947 | −28.583 | 1.00 | 53.78 | E |
| ATOM | 16989 | CD2 | LEU | E | 320 | 64.843 | −19.597 | −27.011 | 1.00 | 53.78 | E |
| ATOM | 16990 | C | LEU | E | 320 | 62.990 | −22.827 | −29.287 | 1.00 | 51.29 | E |
| ATOM | 16991 | O | LEU | E | 320 | 62.238 | −23.255 | −28.401 | 1.00 | 51.29 | E |
| ATOM | 16992 | N | ASP | E | 321 | 62.896 | −23.223 | −30.556 | 1.00 | 60.96 | E |
| ATOM | 16993 | CA | ASP | E | 321 | 61.891 | −24.205 | −30.957 | 1.00 | 60.96 | E |
| ATOM | 16994 | CB | ASP | E | 321 | 61.751 | −24.266 | −32.479 | 1.00 | 116.87 | E |
| ATOM | 16995 | CG | ASP | E | 321 | 61.083 | −23.037 | −33.052 | 1.00 | 95.87 | E |
| ATOM | 16996 | OD1 | ASP | E | 321 | 60.068 | −22.594 | −32.471 | 1.00 | 95.87 | E |
| ATOM | 16997 | OD2 | ASP | E | 321 | 61.564 | −22.524 | −34.090 | 1.00 | 95.87 | E |
| ATOM | 16998 | C | ASP | E | 321 | 62.289 | −25.579 | −30.423 | 1.00 | 60.96 | E |
| ATOM | 16999 | O | ASP | E | 321 | 61.432 | −26.368 | −30.016 | 1.00 | 60.96 | E |
| ATOM | 17000 | N | TYR | E | 322 | 63.591 | −25.866 | −30.423 | 1.00 | 48.86 | E |
| ATOM | 17001 | CA | TYR | E | 322 | 64.073 | −27.150 | −29.928 | 1.00 | 48.86 | E |
| ATOM | 17002 | CB | TYR | E | 322 | 65.586 | −27.276 | −30.088 | 1.00 | 47.50 | E |
| ATOM | 17003 | CG | TYR | E | 322 | 66.062 | −27.564 | −31.495 | 1.00 | 38.21 | E |
| ATOM | 17004 | CD1 | TYR | E | 322 | 65.987 | −26.593 | −32.491 | 1.00 | 38.21 | E |
| ATOM | 17005 | CE1 | TYR | E | 322 | 66.479 | −26.840 | −33.774 | 1.00 | 38.21 | E |
| ATOM | 17006 | CD2 | TYR | E | 322 | 66.634 | −28.797 | −31.824 | 1.00 | 38.21 | E |
| ATOM | 17007 | CE2 | TYR | E | 322 | 67.127 | −29.050 | −33.101 | 1.00 | 38.21 | E |
| ATOM | 17008 | CZ | TYR | E | 322 | 67.052 | −28.069 | −34.065 | 1.00 | 38.21 | E |
| ATOM | 17009 | OH | TYR | E | 322 | 67.586 | −28.284 | −35.313 | 1.00 | 38.21 | E |
| ATOM | 17010 | C | TYR | E | 322 | 63.738 | −27.255 | −28.455 | 1.00 | 48.86 | E |
| ATOM | 17011 | O | TYR | E | 322 | 63.229 | −28.276 | −27.987 | 1.00 | 48.86 | E |
| ATOM | 17012 | N | GLN | E | 323 | 64.033 | −26.184 | −27.725 | 1.00 | 51.21 | E |
| ATOM | 17013 | CA | GLN | E | 323 | 63.776 | −26.143 | −26.297 | 1.00 | 51.21 | E |
| ATOM | 17014 | CB | GLN | E | 323 | 64.108 | −24.769 | −25.746 | 1.00 | 84.50 | E |
| ATOM | 17015 | CG | GLN | E | 323 | 63.855 | −24.648 | −24.272 | 1.00 | 58.89 | E |
| ATOM | 17016 | CD | GLN | E | 323 | 64.324 | −23.319 | −23.717 | 1.00 | 58.89 | E |
| ATOM | 17017 | OE1 | GLN | E | 323 | 63.970 | −22.252 | −24.234 | 1.00 | 58.89 | E |
| ATOM | 17018 | NE2 | GLN | E | 323 | 65.118 | −23.373 | −22.655 | 1.00 | 58.89 | E |
| ATOM | 17019 | C | GLN | E | 323 | 62.330 | −26.485 | −25.990 | 1.00 | 51.21 | E |
| ATOM | 17020 | O | GLN | E | 323 | 62.067 | −27.286 | −25.103 | 1.00 | 51.21 | E |
| ATOM | 17021 | N | LYS | E | 324 | 61.395 | −25.883 | −26.724 | 1.00 | 53.23 | E |
| ATOM | 17022 | CA | LYS | E | 324 | 59.969 | −26.152 | −26.523 | 1.00 | 53.23 | E |
| ATOM | 17023 | CB | LYS | E | 324 | 59.107 | −25.380 | −27.529 | 1.00 | 77.79 | E |
| ATOM | 17024 | CG | LYS | E | 324 | 58.916 | −23.916 | −27.233 | 1.00 | 67.22 | E |
| ATOM | 17025 | CD | LYS | E | 324 | 57.835 | −23.340 | −28.129 | 1.00 | 67.22 | E |
| ATOM | 17026 | CE | LYS | E | 324 | 57.583 | −21.868 | −27.823 | 1.00 | 67.22 | E |
| ATOM | 17027 | NZ | LYS | E | 324 | 56.414 | −21.299 | −28.566 | 1.00 | 67.22 | E |
| ATOM | 17028 | C | LYS | E | 324 | 59.645 | −27.632 | −26.665 | 1.00 | 53.23 | E |
| ATOM | 17029 | O | LYS | E | 324 | 59.026 | −28.236 | −25.789 | 1.00 | 53.23 | E |
| ATOM | 17030 | N | MET | E | 325 | 60.056 | −28.220 | −27.777 | 1.00 | 52.32 | E |
| ATOM | 17031 | CA | MET | E | 325 | 59.769 | −29.621 | −28.014 | 1.00 | 52.32 | E |
| ATOM | 17032 | CB | MET | E | 325 | 59.992 | −29.954 | −29.482 | 1.00 | 72.23 | E |
| ATOM | 17033 | CG | MET | E | 325 | 59.051 | −29.227 | −30.402 | 1.00 | 60.79 | E |
| ATOM | 17034 | SD | MET | E | 325 | 59.347 | −29.645 | −32.118 | 1.00 | 60.79 | E |
| ATOM | 17035 | CE | MET | E | 325 | 60.339 | −28.262 | −32.633 | 1.00 | 60.79 | E |
| ATOM | 17036 | C | MET | E | 325 | 60.578 | −30.572 | −27.151 | 1.00 | 52.32 | E |
| ATOM | 17037 | O | MET | E | 325 | 60.269 | −31.754 | −27.067 | 1.00 | 52.32 | E |
| ATOM | 17038 | N | ALA | E | 326 | 61.616 | −30.075 | −26.506 | 1.00 | 44.60 | E |
| ATOM | 17039 | CA | ALA | E | 326 | 62.423 | −30.955 | −25.684 | 1.00 | 44.60 | E |
| ATOM | 17040 | CB | ALA | E | 326 | 63.901 | −30.560 | −25.798 | 1.00 | 35.85 | E |
| ATOM | 17041 | C | ALA | E | 326 | 61.979 | −30.933 | −24.222 | 1.00 | 44.60 | E |
| ATOM | 17042 | O | ALA | E | 326 | 62.199 | −31.905 | −23.493 | 1.00 | 44.60 | E |
| ATOM | 17043 | N | LYS | E | 327 | 61.337 | −29.837 | −23.817 | 1.00 | 76.60 | E |
| ATOM | 17044 | CA | LYS | E | 327 | 60.899 | −29.642 | −22.442 | 1.00 | 76.60 | E |
| ATOM | 17045 | CB | LYS | E | 327 | 59.581 | −28.880 | −22.388 | 1.00 | 160.18 | E |
| ATOM | 17046 | CG | LYS | E | 327 | 59.275 | −28.392 | −20.984 | 1.00 | 78.47 | E |
| ATOM | 17047 | CD | LYS | E | 327 | 58.037 | −27.509 | −20.934 | 1.00 | 78.47 | E |
| ATOM | 17048 | CE | LYS | E | 327 | 57.796 | −26.957 | −19.528 | 1.00 | 78.47 | E |
| ATOM | 17049 | NZ | LYS | E | 327 | 56.565 | −26.110 | −19.452 | 1.00 | 78.47 | E |
| ATOM | 17050 | C | LYS | E | 327 | 60.777 | −30.901 | −21.605 | 1.00 | 76.60 | E |
| ATOM | 17051 | O | LYS | E | 327 | 61.550 | −31.108 | −20.664 | 1.00 | 76.60 | E |
| ATOM | 17052 | N | GLY | E | 328 | 59.826 | −31.761 | −21.938 | 1.00 | 53.17 | E |
| ATOM | 17053 | CA | GLY | E | 328 | 59.674 | −32.958 | −21.133 | 1.00 | 53.17 | E |
| ATOM | 17054 | C | GLY | E | 328 | 60.497 | −34.190 | −21.482 | 1.00 | 53.17 | E |
| ATOM | 17055 | O | GLY | E | 328 | 60.730 | −35.037 | −20.625 | 1.00 | 53.17 | E |
| ATOM | 17056 | N | LYS | E | 329 | 60.966 | −34.299 | −22.718 | 1.00 | 61.08 | E |
| ATOM | 17057 | CA | LYS | E | 329 | 61.694 | −35.495 | −23.109 | 1.00 | 61.08 | E |
| ATOM | 17058 | CB | LYS | E | 329 | 61.342 | −35.838 | −24.559 | 1.00 | 182.46 | E |
| ATOM | 17059 | CG | LYS | E | 329 | 59.883 | −36.225 | −24.753 | 1.00 | 182.46 | E |
| ATOM | 17060 | CD | LYS | E | 329 | 59.697 | −36.965 | −26.062 | 1.00 | 182.46 | E |
| ATOM | 17061 | CE | LYS | E | 329 | 58.272 | −37.445 | −26.257 | 1.00 | 72.22 | E |
| ATOM | 17062 | NZ | LYS | E | 329 | 58.148 | −38.201 | −27.534 | 1.00 | 72.22 | E |
| ATOM | 17063 | C | LYS | E | 329 | 63.209 | −35.614 | −22.911 | 1.00 | 61.08 | E |
| ATOM | 17064 | O | LYS | E | 329 | 63.828 | −36.501 | −23.494 | 1.00 | 61.08 | E |
| ATOM | 17065 | N | ILE | E | 330 | 63.823 | −34.757 | −22.101 | 1.00 | 45.30 | E |
| ATOM | 17066 | CA | ILE | E | 330 | 65.277 | −34.881 | −21.881 | 1.00 | 45.30 | E |
| ATOM | 17067 | CB | ILE | E | 330 | 66.109 | −33.878 | −22.757 | 1.00 | 52.51 | E |
| ATOM | 17068 | CG2 | ILE | E | 330 | 65.750 | −34.038 | −24.236 | 1.00 | 53.43 | E |
| ATOM | 17069 | CG1 | ILE | E | 330 | 65.863 | −32.445 | −22.297 | 1.00 | 53.43 | E |
| ATOM | 17070 | CD1 | ILE | E | 330 | 66.876 | −31.471 | −22.832 | 1.00 | 53.43 | E |
| ATOM | 17071 | C | ILE | E | 330 | 65.621 | −34.657 | −20.410 | 1.00 | 45.30 | E |
| ATOM | 17072 | O | ILE | E | 330 | 64.765 | −34.244 | −19.633 | 1.00 | 45.30 | E |
| ATOM | 17073 | N | PRO | E | 331 | 66.878 | −34.930 | −20.009 | 1.00 | 54.08 | E |
| ATOM | 17074 | CD | PRO | E | 331 | 68.015 | −35.375 | −20.832 | 1.00 | 34.29 | E |
| ATOM | 17075 | CA | PRO | E | 331 | 67.307 | −34.749 | −18.617 | 1.00 | 54.08 | E |
| ATOM | 17076 | CB | PRO | E | 331 | 68.823 | −34.823 | −18.710 | 1.00 | 178.27 | E |
| ATOM | 17077 | CG | PRO | E | 331 | 69.027 | −35.803 | −19.756 | 1.00 | 34.29 | E |
| ATOM | 17078 | C | PRO | E | 331 | 66.846 | −33.420 | −18.055 | 1.00 | 54.08 | E |
| ATOM | 17079 | O | PRO | E | 331 | 66.945 | −32.386 | −18.721 | 1.00 | 54.08 | E |
| ATOM | 17080 | N | LYS | E | 332 | 66.333 | −33.440 | −16.832 | 1.00 | 56.35 | E |
| ATOM | 17081 | CA | LYS | E | 332 | 65.865 | −32.211 | −16.194 | 1.00 | 56.97 | E |
| ATOM | 17082 | CB | LYS | E | 332 | 65.052 | −32.549 | −14.949 | 1.00 | 104.38 | E |
| ATOM | 17083 | CG | LYS | E | 332 | 65.863 | −33.303 | −13.920 | 1.00 | 75.92 | E |
| ATOM | 17084 | CD | LYS | E | 332 | 65.249 | −33.212 | −12.552 | 1.00 | 75.92 | E |
| ATOM | 17085 | CE | LYS | E | 332 | 66.176 | −33.796 | −11.508 | 1.00 | 75.92 | E |
| ATOM | 17086 | NZ | LYS | E | 332 | 65.632 | −33.591 | −10.134 | 1.00 | 75.92 | E |
| ATOM | 17007 | C | LYS | E | 332 | 67.060 | −31.337 | −15.790 | 1.00 | 56.04 | E |
| ATOM | 17088 | O | LYS | E | 332 | 68.182 | −31.827 | −15.624 | 1.00 | 54.13 | E |
| ATOM | 17089 | N | ASP | E | 333 | 66.817 | −30.043 | −15.631 | 1.00 | 62.79 | E |
| ATOM | 17090 | CA | ASP | E | 333 | 67.880 | −29.134 | −15.229 | 1.00 | 62.25 | E |
| ATOM | 17091 | CB | ASP | E | 333 | 67.362 | −27.698 | −15.194 | 1.00 | 176.17 | E |
| ATOM | 17092 | CG | ASP | E | 333 | 67.525 | −26.991 | −16.519 | 1.00 | 143.76 | E |
| ATOM | 17093 | OD1 | ASP | E | 333 | 67.341 | −27.644 | −17.567 | 1.00 | 143.76 | E |
| ATOM | 17094 | OD2 | ASP | E | 333 | 67.829 | −25.778 | −16.514 | 1.00 | 143.76 | E |
| ATOM | 17095 | C | ASP | E | 333 | 68.376 | −29.535 | −13.852 | 1.00 | 66.10 | E |
| ATOM | 17096 | O | ASP | E | 333 | 67.596 | −29.937 | −12.994 | 1.00 | 65.75 | E |
| ATOM | 17097 | N | THR | E | 334 | 69.680 | −29.434 | −13.644 | 1.00 | 62.13 | E |
| ATOM | 17098 | CA | THR | E | 334 | 70.269 | −29.777 | −12.360 | 1.00 | 61.85 | E |
| ATOM | 17099 | CB | THR | E | 334 | 70.887 | −31.180 | −12.402 | 1.00 | 68.32 | E |
| ATOM | 17100 | OG1 | THR | E | 334 | 69.829 | −32.145 | −12.395 | 1.00 | 54.22 | E |
| ATOM | 17101 | CG2 | THR | E | 334 | 71.817 | −31.411 | −11.212 | 1.00 | 54.22 | E |
| ATOM | 17102 | C | THR | E | 334 | 71.322 | −28.760 | −11.969 | 1.00 | 68.29 | E |
| ATOM | 17103 | O | THR | E | 334 | 72.303 | −28.548 | −12.674 | 1.00 | 65.65 | E |
| ATOM | 17104 | N | THR | E | 335 | 71.108 | −28.129 | −10.829 | 1.00 | 51.03 | E |
| ATOM | 17105 | CA | THR | E | 335 | 72.037 | −27.128 | −10.354 | 1.00 | 50.82 | E |
| ATOM | 17106 | CB | THR | E | 335 | 71.393 | −26.257 | −9.349 | 1.00 | 62.64 | E |
| ATOM | 17107 | OG1 | THR | E | 335 | 70.069 | −25.908 | −9.782 | 1.00 | 52.00 | E |
| ATOM | 17108 | CG2 | THR | E | 335 | 72.238 | −25.000 | −9.166 | 1.00 | 52.00 | E |
| ATOM | 17109 | C | THR | E | 335 | 73.293 | −27.703 | −9.732 | 1.00 | 51.68 | E |
| ATOM | 17110 | O | THR | E | 335 | 73.243 | −28.613 | −8.913 | 1.00 | 53.03 | E |
| ATOM | 17111 | N | PRO | E | 336 | 74.446 | −27.144 | −10.090 | 1.00 | 50.82 | E |
| ATOM | 17112 | CD | PRO | E | 336 | 74.661 | −26.044 | −11.044 | 1.00 | 28.60 | E |
| ATOM | 17113 | CA | PRO | E | 336 | 75.721 | −27.623 | −9.566 | 1.00 | 50.40 | E |
| ATOM | 17114 | CB | PRO | E | 336 | 76.698 | −27.152 | −10.621 | 1.00 | 37.46 | E |
| ATOM | 17115 | CG | PRO | E | 336 | 76.155 | −25.786 | −10.922 | 1.00 | 28.60 | E |
| ATOM | 17116 | C | PRO | E | 336 | 76.110 | −27.103 | −8.195 | 1.00 | 50.44 | E |
| ATOM | 17117 | O | PRO | E | 336 | 75.462 | −26.240 | −7.629 | 1.00 | 51.07 | E |
| ATOM | 17118 | N | THR | E | 337 | 77.200 | −27.656 | −7.688 | 1.00 | 53.06 | E |
| ATOM | 17119 | CA | THR | E | 337 | 77.780 | −27.281 | −6.413 | 1.00 | 56.52 | E |
| ATOM | 17120 | CB | THR | E | 337 | 78.031 | −28.515 | −5.544 | 1.00 | 105.85 | E |
| ATOM | 17121 | OG1 | THR | E | 337 | 76.781 | −29.002 | −5.055 | 1.00 | 56.74 | E |
| ATOM | 17122 | CG2 | THR | E | 337 | 78.931 | −28.179 | −4.375 | 1.00 | 56.74 | E |
| ATOM | 17123 | C | THR | E | 337 | 79.121 | −26.676 | −6.815 | 1.00 | 64.09 | E |
| ATOM | 17124 | O | THR | E | 337 | 79.855 | −27.260 | −7.617 | 1.00 | 55.47 | E |
| ATOM | 17125 | N | VAL | E | 338 | 79.447 | −25.505 | −6.283 | 1.00 | 39.05 | E |
| ATOM | 17126 | CA | VAL | E | 338 | 80.711 | −24.884 | −6.644 | 1.00 | 45.91 | E |
| ATOM | 17127 | CB | VAL | E | 338 | 80.496 | −23.527 | −7.336 | 1.00 | 36.43 | E |
| ATOM | 17128 | CG1 | VAL | E | 338 | 81.809 | −23.020 | −7.907 | 1.00 | 36.05 | E |
| ATOM | 17129 | CG2 | VAL | E | 338 | 79.454 | −23.665 | −8.432 | 1.00 | 36.05 | E |
| ATOM | 17130 | C | VAL | E | 338 | 81.588 | −24.669 | −5.431 | 1.00 | 50.38 | E |
| ATOM | 17131 | O | VAL | E | 338 | 81.090 | −24.422 | −4.331 | 1.00 | 50.21 | E |
| ATOM | 17132 | N | LYS | E | 339 | 82.897 | −24.770 | −5.645 | 1.00 | 53.79 | E |
| ATOM | 17133 | CA | LYS | E | 339 | 83.890 | −24.585 | −4.597 | 1.00 | 51.67 | E |
| ATOM | 17134 | CB | LYS | E | 339 | 84.151 | −25.907 | −3.867 | 1.00 | 140.15 | E |
| ATOM | 17135 | CG | LYS | E | 339 | 82.950 | −26.421 | −3.088 | 1.00 | 108.46 | E |
| ATOM | 17136 | CD | LYS | E | 339 | 82.888 | −27.947 | −3.048 | 1.00 | 108.46 | E |
| ATOM | 17137 | CE | LYS | E | 339 | 81.584 | −28.428 | −2.411 | 1.00 | 108.46 | E |
| ATOM | 17138 | NZ | LYS | E | 339 | 81.389 | −29.898 | −2.532 | 1.00 | 108.46 | E |
| ATOM | 17139 | C | LYS | E | 339 | 85.145 | −24.131 | −5.304 | 1.00 | 60.66 | E |
| ATOM | 17140 | O | LYS | E | 339 | 85.527 | −24.708 | −6.322 | 1.00 | 53.32 | E |
| ATOM | 17141 | N | VAL | E | 340 | 85.773 | −23.082 | −4.791 | 1.00 | 55.81 | E |
| ATOM | 17142 | CA | VAL | E | 340 | 87.007 | −22.577 | −5.393 | 1.00 | 53.24 | E |
| ATOM | 17143 | CB | VAL | E | 340 | 86.976 | −21.048 | −5.572 | 1.00 | 50.51 | E |
| ATOM | 17144 | CG1 | VAL | E | 340 | 88.292 | −20.587 | −6.166 | 1.00 | 35.97 | E |
| ATOM | 17145 | CG2 | VAL | E | 340 | 85.797 | −20.636 | −6.454 | 1.00 | 35.97 | E |
| ATOM | 17146 | C | VAL | E | 340 | 88.163 | −22.903 | −4.456 | 1.00 | 55.81 | E |
| ATOM | 17147 | O | VAL | E | 340 | 88.134 | −22.507 | −3.304 | 1.00 | 52.66 | E |
| ATOM | 17148 | N | THR | E | 341 | 89.179 | −23.612 | −4.937 | 1.00 | 84.00 | E |
| ATOM | 17149 | CA | THR | E | 341 | 90.302 | −23.966 | −4.075 | 1.00 | 79.90 | E |
| ATOM | 17150 | CB | THR | E | 341 | 90.566 | −25.479 | −4.111 | 1.00 | 123.55 | E |
| ATOM | 17151 | OG1 | THR | E | 341 | 89.395 | −26.173 | −3.669 | 1.00 | 104.91 | E |
| ATOM | 17152 | CG2 | THR | E | 341 | 91.733 | −25.840 | −3.205 | 1.00 | 104.91 | E |
| ATOM | 17153 | C | THR | E | 341 | 91.581 | −23.238 | −4.456 | 1.00 | 82.05 | E |
| ATOM | 17154 | O | THR | E | 341 | 91.847 | −23.000 | −5.633 | 1.00 | 83.65 | E |
| ATOM | 17155 | N | GLY | E | 342 | 92.375 | −22.892 | −3.447 | 1.00 | 73.12 | E |
| ATOM | 17156 | CA | GLY | E | 342 | 93.622 | −22.198 | −3.693 | 1.00 | 77.29 | E |
| ATOM | 17157 | C | GLY | E | 342 | 94.842 | −23.067 | −3.463 | 1.00 | 76.26 | E |
| ATOM | 17158 | O | GLY | E | 342 | 95.629 | −22.805 | −2.567 | 1.00 | 70.53 | E |
| ATOM | 17159 | N | ASN | E | 343 | 95.002 | −24.110 | −4.271 | 1.00 | 124.86 | E |
| ATOM | 17160 | CA | ASN | E | 343 | 96.153 | −25.001 | −4.147 | 1.00 | 131.27 | E |
| ATOM | 17161 | CB | ASN | E | 343 | 95.989 | −26.218 | −5.066 | 1.00 | 189.12 | E |
| ATOM | 17162 | CG | ASN | E | 343 | 94.670 | −26.939 | −4.857 | 1.00 | 168.92 | E |
| ATOM | 17163 | OD1 | ASN | E | 343 | 94.396 | −27.458 | −3.774 | 1.00 | 168.92 | E |
| ATOM | 17164 | ND2 | ASN | E | 343 | 93.845 | −26.974 | −5.898 | 1.00 | 168.92 | E |
| ATOM | 17165 | C | ASN | E | 343 | 97.387 | −24.215 | −4.572 | 1.00 | 129.33 | E |
| ATOM | 17166 | O | ASN | E | 343 | 97.349 | −22.988 | −4.666 | 1.00 | 117.96 | E |
| ATOM | 17167 | N | GLU | E | 344 | 98.479 | −24.923 | −4.831 | 1.00 | 200.84 | E |
| ATOM | 17168 | CA | GLU | E | 344 | 99.709 | −24.281 | −5.273 | 1.00 | 200.84 | E |
| ATOM | 17169 | CB | GLU | E | 344 | 100.385 | −23.556 | −4.103 | 1.00 | 112.33 | E |
| ATOM | 17170 | CG | GLU | E | 344 | 99.908 | −22.113 | −3.959 | 1.00 | 92.23 | E |
| ATOM | 17171 | CD | GLU | E | 344 | 100.441 | −21.426 | −2.722 | 1.00 | 92.23 | E |
| ATOM | 17172 | OE1 | GLU | E | 344 | 99.956 | −21.737 | −1.616 | 1.00 | 92.23 | E |
| ATOM | 17173 | OE2 | GLU | E | 344 | 101.346 | −20.578 | −2.853 | 1.00 | 92.23 | E |
| ATOM | 17174 | C | GLU | E | 344 | 100.651 | −25.283 | −5.927 | 1.00 | 200.84 | E |
| ATOM | 17175 | O | GLU | E | 344 | 101.238 | −26.135 | −5.264 | 1.00 | 200.84 | E |
| ATOM | 17176 | N | LEU | E | 345 | 100.780 | −25.149 | −7.245 | 1.00 | 200.84 | E |
| ATOM | 17177 | CA | LEU | E | 345 | 101.598 | −26.025 | −8.069 | 1.00 | 200.84 | E |
| ATOM | 17178 | CB | LEU | E | 345 | 101.181 | −25.861 | −9.536 | 1.00 | 200.84 | E |
| ATOM | 17179 | CG | LEU | E | 345 | 99.737 | −26.242 | −9.922 | 1.00 | 154.50 | E |
| ATOM | 17180 | CD1 | LEU | E | 345 | 99.570 | −26.113 | −11.431 | 1.00 | 154.50 | E |
| ATOM | 17181 | CD2 | LEU | E | 345 | 99.408 | −27.668 | −9.481 | 1.00 | 154.50 | E |
| ATOM | 17182 | C | LEU | E | 345 | 103.131 | −25.942 | −7.934 | 1.00 | 200.84 | E |
| ATOM | 17183 | O | LEU | E | 345 | 103.863 | −26.143 | −8.903 | 1.00 | 200.84 | E |
| ATOM | 17184 | N | GLU | E | 346 | 103.605 | −25.656 | −6.726 | 1.00 | 200.84 | E |
| ATOM | 17185 | CA | GLU | E | 346 | 105.035 | −25.594 | −6.423 | 1.00 | 200.84 | E |
| ATOM | 17186 | CB | GLU | E | 346 | 105.643 | −26.976 | −6.651 | 1.00 | 200.84 | E |
| ATOM | 17187 | CG | GLU | E | 346 | 104.759 | −28.096 | −6.121 | 1.00 | 200.84 | E |
| ATOM | 17188 | CD | GLU | E | 346 | 105.478 | −29.411 | −5.846 | 1.00 | 200.84 | E |
| ATOM | 17189 | OE1 | GLU | E | 346 | 106.326 | −29.448 | −4.925 | 1.00 | 200.84 | E |
| ATOM | 17190 | OE2 | GLU | E | 346 | 105.210 | −30.401 | −6.560 | 1.00 | 200.84 | E |
| ATOM | 17191 | C | GLU | E | 346 | 105.928 | −24.530 | −7.072 | 1.00 | 200.84 | E |
| ATOM | 17192 | O | GLU | E | 346 | 106.331 | −23.572 | −6.404 | 1.00 | 200.84 | E |
| ATOM | 17193 | N | ASP | E | 347 | 106.259 | −24.697 | −8.353 | 1.00 | 200.84 | E |
| ATOM | 17194 | CA | ASP | E | 347 | 107.125 | −23.733 | −9.037 | 1.00 | 200.84 | E |
| ATOM | 17195 | CB | ASP | E | 347 | 107.449 | −24.206 | −10.465 | 1.00 | 161.62 | E |
| ATOM | 17196 | CG | ASP | E | 347 | 106.356 | −23.864 | −11.463 | 1.00 | 161.62 | E |
| ATOM | 17197 | OD1 | ASP | E | 347 | 105.218 | −24.356 | −11.304 | 1.00 | 161.62 | E |
| ATOM | 17198 | OD2 | ASP | E | 347 | 106.638 | −23.100 | −12.410 | 1.00 | 161.62 | E |
| ATOM | 17199 | C | ASP | E | 347 | 106.517 | −22.328 | −9.077 | 1.00 | 200.84 | E |
| ATOM | 17200 | O | ASP | E | 347 | 107.229 | −21.330 | −8.943 | 1.00 | 200.84 | E |
| ATOM | 17201 | N | GLY | E | 348 | 105.200 | −22.256 | −9.260 | 1.00 | 200.84 | E |
| ATOM | 17202 | CA | GLY | E | 348 | 104.522 | −20.972 | −9.303 | 1.00 | 200.84 | E |
| ATOM | 17203 | C | GLY | E | 348 | 103.899 | −20.627 | −7.963 | 1.00 | 200.84 | E |
| ATOM | 17204 | O | GLY | E | 348 | 103.483 | −21.516 | −7.219 | 1.00 | 196.32 | E |
| ATOM | 17205 | N | ASN | E | 349 | 103.824 | −19.337 | −7.647 | 1.00 | 200.39 | E |
| ATOM | 17206 | CA | ASN | E | 349 | 103.253 | −18.907 | −6.376 | 1.00 | 200.29 | E |
| ATOM | 17207 | CB | ASN | E | 349 | 103.136 | −17.374 | −6.337 | 1.00 | 107.13 | E |
| ATOM | 17208 | CG | ASN | E | 349 | 102.784 | −16.775 | −7.686 | 1.00 | 92.31 | E |
| ATOM | 17209 | OD1 | ASN | E | 349 | 103.543 | −16.886 | −8.657 | 1.00 | 92.31 | E |
| ATOM | 17210 | ND2 | ASN | E | 349 | 101.631 | −16.129 | −7.752 | 1.00 | 92.31 | E |
| ATOM | 17211 | C | ASN | E | 349 | 101.912 | −19.563 | −6.027 | 1.00 | 200.39 | E |
| ATOM | 17212 | O | ASN | E | 349 | 101.888 | −20.560 | −5.303 | 1.00 | 200.39 | E |
| ATOM | 17213 | N | MET | E | 350 | 100.806 | −19.026 | −6.543 | 1.00 | 132.02 | E |
| ATOM | 17214 | CA | MET | E | 350 | 99.484 | −19.576 | −6.236 | 1.00 | 128.11 | E |
| ATOM | 17215 | CB | MET | E | 350 | 98.676 | −18.538 | −5.460 | 1.00 | 153.51 | E |
| ATOM | 17216 | CG | MET | E | 350 | 97.280 | −18.987 | −5.080 | 1.00 | 108.19 | E |
| ATOM | 17217 | SD | MET | E | 350 | 96.430 | −17.774 | −4.053 | 1.00 | 108.19 | E |
| ATOM | 17218 | CE | MET | E | 350 | 96.608 | −18.557 | −2.420 | 1.00 | 108.19 | E |
| ATOM | 17219 | C | MET | E | 350 | 98.663 | −20.066 | −7.434 | 1.00 | 125.25 | E |
| ATOM | 17220 | O | MET | E | 350 | 98.860 | −19.618 | −8.560 | 1.00 | 121.68 | E |
| ATOM | 17221 | N | THR | E | 351 | 97.736 | −20.987 | −7.174 | 1.00 | 84.05 | E |
| ATOM | 17222 | CA | THR | E | 351 | 96.877 | −21.538 | −8.216 | 1.00 | 82.01 | E |
| ATOM | 17223 | CB | THR | E | 351 | 97.339 | −22.967 | −8.645 | 1.00 | 103.76 | E |
| ATOM | 17224 | OG1 | THR | E | 351 | 97.055 | −23.158 | −10.032 | 1.00 | 70.88 | E |
| ATOM | 17225 | CG2 | THR | E | 351 | 96.601 | −24.052 | −7.869 | 1.00 | 70.88 | E |
| ATOM | 17226 | C | THR | E | 351 | 95.434 | −21.609 | −7.719 | 1.00 | 76.24 | E |
| ATOM | 17227 | O | THR | E | 351 | 95.172 | −22.023 | −6.591 | 1.00 | 67.83 | E |
| ATOM | 17228 | N | LEU | E | 352 | 94.497 | −21.188 | −8.557 | 1.00 | 59.43 | E |
| ATOM | 17229 | CA | LEU | E | 352 | 93.089 | −21.235 | −8.192 | 1.00 | 62.45 | E |
| ATOM | 17230 | CB | LEU | E | 352 | 92.453 | −19.858 | −8.367 | 1.00 | 81.68 | E |
| ATOM | 17231 | CG | LEU | E | 352 | 92.789 | −18.939 | −7.194 | 1.00 | 62.74 | E |
| ATOM | 17232 | CD1 | LEU | E | 352 | 92.397 | −17.518 | −7.503 | 1.00 | 62.74 | E |
| ATOM | 17233 | CD2 | LEU | E | 352 | 92.063 | −19.439 | −5.946 | 1.00 | 62.74 | E |
| ATOM | 17234 | C | LEU | E | 352 | 92.347 | −22.280 | −9.014 | 1.00 | 59.13 | E |
| ATOM | 17235 | O | LEU | E | 352 | 92.541 | −22.391 | −10.227 | 1.00 | 54.75 | E |
| ATOM | 17236 | N | GLU | E | 353 | 91.502 | −23.050 | −8.338 | 1.00 | 63.36 | E |
| ATOM | 17237 | CA | GLU | E | 353 | 90.740 | −24.097 | −8.992 | 1.00 | 59.61 | E |
| ATOM | 17238 | CB | GLU | E | 353 | 91.258 | −25.459 | −8.559 | 1.00 | 187.56 | E |
| ATOM | 17239 | CG | GLU | E | 353 | 90.551 | −26.612 | −9.218 | 1.00 | 146.11 | E |
| ATOM | 17240 | CD | GLU | E | 353 | 91.039 | −27.938 | −8.696 | 1.00 | 146.11 | E |
| ATOM | 17241 | OE1 | GLU | E | 353 | 92.259 | −28.191 | −8.779 | 1.00 | 146.11 | E |
| ATOM | 17242 | OE2 | GLU | E | 353 | 90.207 | −28.725 | −8.199 | 1.00 | 146.11 | E |
| ATOM | 17243 | C | GLU | E | 353 | 89.255 | −23.997 | −8.682 | 1.00 | 62.86 | E |
| ATOM | 17244 | O | GLU | E | 353 | 88.831 | −24.165 | −7.539 | 1.00 | 63.64 | E |
| ATOM | 17245 | N | CYS | E | 354 | 88.473 | −23.723 | −9.717 | 1.00 | 76.30 | E |
| ATOM | 17246 | CA | CYS | E | 354 | 87.030 | −23.600 | −9.592 | 1.00 | 76.58 | E |
| ATOM | 17247 | C | CYS | E | 354 | 86.459 | −24.930 | −10.041 | 1.00 | 77.67 | E |
| ATOM | 17248 | O | CYS | E | 354 | 86.715 | −25.359 | −11.163 | 1.00 | 76.37 | E |
| ATOM | 17249 | CB | CYS | E | 354 | 86.528 | −22.491 | −10.515 | 1.00 | 111.67 | E |
| ATOM | 17250 | SG | CYS | E | 354 | 84.837 | −21.891 | −10.210 | 1.00 | 88.86 | E |
| ATOM | 17251 | N | THR | E | 355 | 85.712 | −25.605 | −9.175 | 1.00 | 61.87 | E |
| ATOM | 17252 | CA | THR | E | 355 | 85.124 | −26.880 | −9.575 | 1.00 | 51.23 | E |
| ATOM | 17253 | CB | THR | E | 355 | 85.667 | −28.059 | −8.755 | 1.00 | 98.05 | E |
| ATOM | 17254 | OG1 | THR | E | 355 | 84.726 | −28.415 | −7.741 | 1.00 | 47.20 | E |
| ATOM | 17255 | CG2 | THR | E | 355 | 86.981 | −27.693 | −8.116 | 1.00 | 47.20 | E |
| ATOM | 17256 | C | THR | E | 355 | 83.614 | −26.829 | −9.437 | 1.00 | 64.72 | E |
| ATOM | 17257 | O | THR | E | 355 | 83.077 | −26.467 | −8.384 | 1.00 | 57.52 | E |
| ATOM | 17258 | N | VAL | E | 356 | 82.942 | −27.185 | −10.526 | 1.00 | 52.96 | E |
| ATOM | 17259 | CA | VAL | E | 356 | 81.491 | −27.171 | −10.580 | 1.00 | 48.68 | E |
| ATOM | 17260 | CB | VAL | E | 356 | 81.021 | −26.377 | −11.807 | 1.00 | 57.79 | E |
| ATOM | 17261 | CG1 | VAL | E | 356 | 79.532 | −26.130 | −11.733 | 1.00 | 42.32 | E |
| ATOM | 17262 | CG2 | VAL | E | 356 | 81.759 | −25.061 | −11.859 | 1.00 | 42.32 | E |
| ATOM | 17263 | C | VAL | E | 356 | 81.024 | −28.610 | −10.641 | 1.00 | 57.52 | E |
| ATOM | 17264 | O | VAL | E | 356 | 81.365 | −29.341 | −11.569 | 1.00 | 58.63 | E |
| ATOM | 17265 | N | ASN | E | 357 | 80.231 | −29.007 | −9.650 | 1.00 | 49.80 | E |
| ATOM | 17266 | CA | ASN | E | 357 | 79.787 | −30.388 | −9.549 | 1.00 | 46.02 | E |
| ATOM | 17267 | CB | ASN | E | 357 | 80.022 | −30.910 | −8.132 | 1.00 | 124.63 | E |
| ATOM | 17268 | CG | ASN | E | 357 | 81.486 | −30.954 | −7.773 | 1.00 | 78.55 | E |
| ATOM | 17269 | OD1 | ASN | E | 357 | 82.292 | −31.550 | −8.490 | 1.00 | 78.55 | E |
| ATOM | 17270 | ND2 | ASN | E | 357 | 81.844 | −30.318 | −6.659 | 1.00 | 78.55 | E |
| ATOM | 17271 | C | ASN | E | 357 | 78.385 | −30.727 | −9.960 | 1.00 | 50.18 | E |
| ATOM | 17272 | O | ASN | E | 357 | 77.410 | −30.131 | −9.494 | 1.00 | 53.06 | E |
| ATOM | 17273 | N | SER | E | 358 | 78.324 | −31.739 | −10.823 | 1.00 | 55.67 | E |
| ATOM | 17274 | CA | SER | E | 358 | 77.100 | −32.287 | −11.386 | 1.00 | 62.97 | E |
| ATOM | 17275 | CB | SER | E | 358 | 76.605 | −33.460 | −10.522 | 1.00 | 63.22 | E |
| ATOM | 17276 | OG | SER | E | 358 | 76.183 | −33.015 | −9.260 | 1.00 | 61.56 | E |
| ATOM | 17277 | C | SER | E | 358 | 75.969 | −31.288 | −11.664 | 1.00 | 67.69 | E |
| ATOM | 17278 | O | SER | E | 358 | 75.102 | −31.022 | −10.822 | 1.00 | 63.24 | E |
| ATOM | 17279 | N | PHE | E | 359 | 76.007 | −30.756 | −12.884 | 1.00 | 40.05 | E |
| ATOM | 17280 | CA | PHE | E | 359 | 75.023 | −29.800 | −13.403 | 1.00 | 34.36 | E |
| ATOM | 17281 | CB | PHE | E | 359 | 75.735 | −28.492 | −13.742 | 1.00 | 47.59 | E |
| ATOM | 17282 | CG | PHE | E | 359 | 77.008 | −28.677 | −14.519 | 1.00 | 42.19 | E |
| ATOM | 17283 | CD1 | PHE | E | 359 | 76.982 | −28.846 | −15.905 | 1.00 | 42.19 | E |
| ATOM | 17284 | CD2 | PHE | E | 359 | 78.242 | −28.645 | −13.872 | 1.00 | 42.19 | E |
| ATOM | 17285 | CE1 | PHE | E | 359 | 78.175 | −28.973 | −16.638 | 1.00 | 42.19 | E |
| ATOM | 17286 | CE2 | PHE | E | 359 | 79.436 | −28.772 | −14.596 | 1.00 | 42.19 | E |
| ATOM | 17287 | CZ | PHE | E | 359 | 79.401 | −28.934 | −15.980 | 1.00 | 42.19 | E |
| ATOM | 17288 | C | PHE | E | 359 | 74.424 | −30.427 | −14.668 | 1.00 | 40.95 | E |
| ATOM | 17289 | O | PHE | E | 359 | 75.147 | −31.126 | −15.382 | 1.00 | 38.50 | E |
| ATOM | 17290 | N | TYR | E | 360 | 73.140 | −30.219 | −14.974 | 1.00 | 95.10 | E |
| ATOM | 17291 | CA | TYR | E | 360 | 72.685 | −30.882 | −16.177 | 1.00 | 94.88 | E |
| ATOM | 17292 | CB | TYR | E | 360 | 71.196 | −31.208 | −16.244 | 1.00 | 156.81 | E |
| ATOM | 17293 | CG | TYR | E | 360 | 70.912 | −31.684 | −17.673 | 1.00 | 43.95 | E |
| ATOM | 17294 | CD1 | TYR | E | 360 | 71.758 | −32.627 | −18.291 | 1.00 | 43.95 | E |
| ATOM | 17295 | CE1 | TYR | E | 360 | 71.629 | −32.954 | −19.639 | 1.00 | 43.95 | E |
| ATOM | 17296 | CD2 | TYR | E | 360 | 69.915 | −31.099 | −18.454 | 1.00 | 43.95 | E |
| ATOM | 17297 | CE2 | TYR | E | 360 | 69.774 | −31.427 | −19.808 | 1.00 | 43.95 | E |
| ATOM | 17298 | CZ | TYR | E | 360 | 70.636 | −32.351 | −20.395 | 1.00 | 43.95 | E |
| ATOM | 17299 | OH | TYR | E | 360 | 70.505 | −32.661 | −21.740 | 1.00 | 43.95 | E |
| ATOM | 17300 | C | TYR | E | 360 | 73.019 | −30.270 | −17.506 | 1.00 | 95.97 | E |
| ATOM | 17301 | O | TYR | E | 360 | 73.967 | −30.706 | −18.138 | 1.00 | 101.90 | E |
| ATOM | 17302 | N | PRO | E | 361 | 72.235 | −29.285 | −17.974 | 1.00 | 63.39 | E |
| ATOM | 17303 | CD | PRO | E | 361 | 71.431 | −28.293 | −17.237 | 1.00 | 43.77 | E |
| ATOM | 17304 | CA | PRO | E | 361 | 72.664 | −28.796 | −19.295 | 1.00 | 58.98 | E |
| ATOM | 17305 | CB | PRO | E | 361 | 71.883 | −27.489 | −19.465 | 1.00 | 43.01 | E |
| ATOM | 17306 | CG | PRO | E | 361 | 71.663 | −27.030 | −18.042 | 1.00 | 43.77 | E |
| ATOM | 17307 | C | PRO | E | 361 | 74.188 | −28.687 | −19.215 | 1.00 | 66.38 | E |
| ATOM | 17308 | O | PRO | E | 361 | 74.736 | −27.926 | −18.424 | 1.00 | 63.55 | E |
| ATOM | 17309 | N | PRO | E | 362 | 74.888 | −29.533 | −19.985 | 1.00 | 44.31 | E |
| ATOM | 17310 | CD | PRO | E | 362 | 74.267 | −30.436 | −20.972 | 1.00 | 34.92 | E |
| ATOM | 17311 | CA | PRO | E | 362 | 76.351 | −29.623 | −20.049 | 1.00 | 38.02 | E |
| ATOM | 17312 | CB | PRO | E | 362 | 76.587 | −30.772 | −21.031 | 1.00 | 34.92 | E |
| ATOM | 17313 | CG | PRO | E | 362 | 75.411 | −30.714 | −21.911 | 1.00 | 34.92 | E |
| ATOM | 17314 | C | PRO | E | 362 | 77.150 | −28.381 | −20.382 | 1.00 | 40.62 | E |
| ATOM | 17315 | O | PRO | E | 362 | 78.359 | −28.346 | −20.154 | 1.00 | 45.38 | E |
| ATOM | 17316 | N | ASP | E | 363 | 76.491 | −27.361 | −20.917 | 1.00 | 39.15 | E |
| ATOM | 17317 | CA | ASP | E | 363 | 77.192 | −26.133 | −21.246 | 1.00 | 39.00 | E |
| ATOM | 17318 | CB | ASP | E | 363 | 76.412 | −25.317 | −22.299 | 1.00 | 67.66 | E |
| ATOM | 17319 | CG | ASP | E | 363 | 74.921 | −25.179 | −21.978 | 1.00 | 61.87 | E |
| ATOM | 17320 | OD1 | ASP | E | 363 | 74.234 | −26.224 | −21.858 | 1.00 | 61.87 | E |
| ATOM | 17321 | OD2 | ASP | E | 363 | 74.437 | −24.023 | −21.860 | 1.00 | 61.87 | E |
| ATOM | 17322 | C | ASP | E | 363 | 77.425 | −25.308 | −19.978 | 1.00 | 42.54 | E |
| ATOM | 17323 | O | ASP | E | 363 | 76.501 | −25.000 | −19.223 | 1.00 | 39.04 | E |
| ATOM | 17324 | N | VAL | E | 364 | 78.680 | −24.980 | −19.727 | 1.00 | 46.84 | E |
| ATOM | 17325 | CA | VAL | E | 364 | 79.036 | −24.170 | −18.576 | 1.00 | 49.16 | E |
| ATOM | 17326 | CB | VAL | E | 364 | 79.500 | −25.022 | −17.385 | 1.00 | 58.48 | E |
| ATOM | 17327 | CG1 | VAL | E | 364 | 80.311 | −24.186 | −16.433 | 1.00 | 36.87 | E |
| ATOM | 17328 | CG2 | VAL | E | 364 | 78.311 | −25.547 | −16.638 | 1.00 | 36.87 | E |
| ATOM | 17329 | C | VAL | E | 364 | 80.177 | −23.280 | −18.994 | 1.00 | 56.50 | E |
| ATOM | 17330 | O | VAL | E | 364 | 81.055 | −23.686 | −19.744 | 1.00 | 48.24 | E |
| ATOM | 17331 | N | ILE | E | 365 | 80.152 | −22.047 | −18.533 | 1.00 | 46.66 | E |
| ATOM | 17332 | CA | ILE | E | 365 | 81.227 | −21.138 | −18.848 | 1.00 | 40.71 | E |
| ATOM | 17333 | CB | ILE | E | 365 | 80.814 | −20.087 | −19.883 | 1.00 | 63.77 | E |
| ATOM | 17334 | CG2 | ILE | E | 365 | 81.915 | −19.041 | −20.026 | 1.00 | 40.21 | E |
| ATOM | 17335 | CG1 | ILE | E | 365 | 80.558 | −20.778 | −21.228 | 1.00 | 40.21 | E |
| ATOM | 17336 | CD1 | ILE | E | 365 | 80.163 | −19.838 | −22.332 | 1.00 | 40.21 | E |
| ATOM | 17337 | C | ILE | E | 365 | 81.675 | −20.482 | −17.564 | 1.00 | 47.47 | E |
| ATOM | 17338 | O | ILE | E | 365 | 80.874 | −20.198 | −16.666 | 1.00 | 48.24 | E |
| ATOM | 17339 | N | THR | E | 366 | 82.976 | −20.254 | −17.489 | 1.00 | 33.98 | E |
| ATOM | 17340 | CA | THR | E | 366 | 83.593 | −19.689 | −16.317 | 1.00 | 42.26 | E |
| ATOM | 17341 | CB | THR | E | 366 | 84.495 | −20.756 | −15.706 | 1.00 | 69.41 | E |
| ATOM | 17342 | OG1 | THR | E | 366 | 84.082 | −21.001 | −14.365 | 1.00 | 57.09 | E |
| ATOM | 17343 | CG2 | THR | E | 366 | 85.949 | −20.339 | −15.744 | 1.00 | 57.09 | E |
| ATOM | 17344 | C | THR | E | 366 | 84.388 | −18.431 | −16.622 | 1.00 | 51.24 | E |
| ATOM | 17345 | O | THR | E | 366 | 85.116 | −18.366 | −17.615 | 1.00 | 50.48 | E |
| ATOM | 17346 | N | LYS | E | 367 | 84.234 | −17.429 | −15.765 | 1.00 | 47.68 | E |
| ATOM | 17347 | CA | LYS | E | 367 | 84.962 | −16.169 | −15.911 | 1.00 | 50.88 | E |
| ATOM | 17348 | CB | LYS | E | 367 | 84.019 | −15.002 | −16.230 | 1.00 | 67.18 | E |
| ATOM | 17349 | CG | LYS | E | 367 | 83.468 | −15.023 | −17.625 | 1.00 | 58.19 | E |
| ATOM | 17350 | CD | LYS | E | 367 | 82.757 | −13.733 | −17.986 | 1.00 | 58.19 | E |
| ATOM | 17351 | CE | LYS | E | 367 | 82.432 | −13.720 | −19.475 | 1.00 | 58.19 | E |
| ATOM | 17352 | NZ | LYS | E | 367 | 82.008 | −12.379 | −19.969 | 1.00 | 58.19 | E |
| ATOM | 17353 | C | LYS | E | 367 | 85.693 | −15.856 | −14.621 | 1.00 | 53.58 | E |
| ATOM | 17354 | O | LYS | E | 367 | 85.100 | −15.905 | −13.536 | 1.00 | 53.89 | E |
| ATOM | 17355 | N | TRP | E | 368 | 86.982 | −15.545 | −14.747 | 1.00 | 50.18 | E |
| ATOM | 17356 | CA | TRP | E | 368 | 87.809 | −15.180 | −13.604 | 1.00 | 50.24 | E |
| ATOM | 17357 | CB | TRP | E | 368 | 89.174 | −15.860 | −13.674 | 1.00 | 81.21 | E |
| ATOM | 17358 | CG | TRP | E | 368 | 89.088 | −17.321 | −13.446 | 1.00 | 61.13 | E |
| ATOM | 17359 | CD2 | TRP | E | 368 | 89.098 | −17.988 | −12.181 | 1.00 | 61.13 | E |
| ATOM | 17360 | CE2 | TRP | E | 368 | 88.943 | −19.369 | −12.435 | 1.00 | 61.13 | E |
| ATOM | 17361 | CE3 | TRP | E | 368 | 89.221 | −17.554 | −10.855 | 1.00 | 61.13 | E |
| ATOM | 17362 | CD1 | TRP | E | 368 | 88.932 | −18.291 | −14.390 | 1.00 | 61.13 | E |
| ATOM | 17363 | NE1 | TRP | E | 368 | 88.845 | −19.524 | −13.793 | 1.00 | 61.13 | E |
| ATOM | 17364 | CZ2 | TRP | E | 368 | 88.909 | −20.324 | −11.409 | 1.00 | 61.13 | E |
| ATOM | 17365 | CZ3 | TRP | E | 368 | 89.185 | −18.506 | −9.832 | 1.00 | 61.13 | E |
| ATOM | 17366 | CH2 | TRP | E | 368 | 89.031 | −19.874 | −10.117 | 1.00 | 61.13 | E |
| ATOM | 17367 | C | TRP | E | 368 | 87.980 | −13.669 | −13.599 | 1.00 | 52.19 | E |
| ATOM | 17368 | O | TRP | E | 368 | 88.564 | −13.096 | −14.513 | 1.00 | 52.38 | E |
| ATOM | 17369 | N | ILE | E | 369 | 87.462 | −13.029 | −12.559 | 1.00 | 55.35 | E |
| ATOM | 17370 | CA | ILE | E | 369 | 87.535 | −11.583 | −12.433 | 1.00 | 56.72 | E |
| ATOM | 17371 | CB | ILE | E | 369 | 86.127 | −10.985 | −12.538 | 1.00 | 67.11 | E |
| ATOM | 17372 | CG2 | ILE | E | 369 | 86.194 | −9.474 | −12.496 | 1.00 | 44.79 | E |
| ATOM | 17373 | CG1 | ILE | E | 369 | 85.471 | −11.461 | −13.834 | 1.00 | 44.79 | E |
| ATOM | 17374 | CD1 | ILE | E | 369 | 84.023 | −11.055 | −13.969 | 1.00 | 44.79 | E |
| ATOM | 17375 | C | ILE | E | 369 | 88.151 | −11.164 | −11.098 | 1.00 | 58.54 | E |
| ATOM | 17376 | O | ILE | E | 369 | 87.808 | −11.720 | −10.054 | 1.00 | 55.34 | E |
| ATOM | 17377 | N | GLU | E | 370 | 89.060 | −10.190 | −11.131 | 1.00 | 55.13 | E |
| ATOM | 17378 | CA | GLU | E | 370 | 89.691 | −9.698 | −9.907 | 1.00 | 57.78 | E |
| ATOM | 17379 | CB | GLU | E | 370 | 90.725 | −8.627 | −10.246 | 1.00 | 114.38 | E |
| ATOM | 17380 | CG | GLU | E | 370 | 92.005 | −9.227 | −10.791 | 1.00 | 93.02 | E |
| ATOM | 17381 | CD | GLU | E | 370 | 92.950 | −8.199 | −11.378 | 1.00 | 93.02 | E |
| ATOM | 17382 | OE1 | GLU | E | 370 | 93.207 | −7.173 | −10.713 | 1.00 | 93.02 | E |
| ATOM | 17383 | OE2 | GLU | E | 370 | 93.448 | −8.426 | −12.504 | 1.00 | 93.02 | E |
| ATOM | 17384 | C | GLU | E | 370 | 88.591 | −9.149 | −8.998 | 1.00 | 57.96 | E |
| ATOM | 17385 | O | GLU | E | 370 | 87.731 | −8.388 | −9.437 | 1.00 | 56.81 | E |
| ATOM | 17386 | N | SER | E | 371 | 88.612 | −9.554 | −7.733 | 1.00 | 60.29 | E |
| ATOM | 17387 | CA | SER | E | 371 | 87.586 | −9.149 | −6.773 | 1.00 | 60.83 | E |
| ATOM | 17388 | CB | SER | E | 371 | 87.863 | −9.795 | −5.413 | 1.00 | 87.61 | E |
| ATOM | 17389 | OG | SER | E | 371 | 89.148 | −9.439 | −4.943 | 1.00 | 76.69 | E |
| ATOM | 17390 | C | SER | E | 371 | 87.388 | −7.650 | −6.591 | 1.00 | 60.10 | E |
| ATOM | 17391 | O | SER | E | 371 | 86.312 | −7.212 | −6.167 | 1.00 | 61.04 | E |
| ATOM | 17392 | N | GLU | E | 372 | 88.415 | −6.861 | −6.902 | 1.00 | 61.38 | E |
| ATOM | 17393 | CA | GLU | E | 372 | 88.308 | −5.414 | −6.747 | 1.00 | 59.93 | E |
| ATOM | 17394 | CB | GLU | E | 372 | 89.581 | −4.728 | −7.231 | 1.00 | 97.63 | E |
| ATOM | 17395 | CG | GLU | E | 372 | 89.919 | −5.042 | −8.662 | 1.00 | 97.63 | E |
| ATOM | 17396 | CD | GLU | E | 372 | 90.964 | −4.110 | −9.237 | 1.00 | 97.63 | E |
| ATOM | 17397 | OE1 | GLU | E | 372 | 90.688 | −2.891 | −9.310 | 1.00 | 97.63 | E |
| ATOM | 17398 | OE2 | GLU | E | 372 | 92.057 | −4.593 | −9.617 | 1.00 | 97.63 | E |
| ATOM | 17399 | C | GLU | E | 372 | 87.112 | −4.881 | −7.528 | 1.00 | 64.54 | E |
| ATOM | 17400 | O | GLU | E | 372 | 86.383 | −4.001 | −7.061 | 1.00 | 65.61 | E |
| ATOM | 17401 | N | HIS | E | 373 | 86.905 | −5.441 | −8.714 | 1.00 | 54.34 | E |
| ATOM | 17402 | CA | HIS | E | 373 | 85.818 | −5.032 | −9.594 | 1.00 | 57.83 | E |
| ATOM | 17403 | CB | HIS | E | 373 | 85.958 | −5.726 | −10.938 | 1.00 | 95.53 | E |
| ATOM | 17404 | CG | HIS | E | 373 | 87.221 | −5.378 | −11.651 | 1.00 | 64.88 | E |
| ATOM | 17405 | CD2 | HIS | E | 373 | 88.223 | −6.154 | −12.134 | 1.00 | 64.88 | E |
| ATOM | 17406 | ND1 | HIS | E | 373 | 87.581 | −4.076 | −11.919 | 1.00 | 64.88 | E |
| ATOM | 17407 | CE1 | HIS | E | 373 | 88.750 | −4.063 | −12.536 | 1.00 | 64.88 | E |
| ATOM | 17408 | NE2 | HIS | E | 373 | 89.161 | −5.311 | −12.678 | 1.00 | 64.88 | E |
| ATOM | 17409 | C | HIS | E | 373 | 84.419 | −5.260 | −9.056 | 1.00 | 53.60 | E |
| ATOM | 17410 | O | HIS | E | 373 | 83.460 | −4.798 | −9.654 | 1.00 | 49.44 | E |
| ATOM | 17411 | N | PHE | E | 374 | 84.285 | −5.971 | −7.941 | 1.00 | 87.86 | E |
| ATOM | 17412 | CA | PHE | E | 374 | 82.959 | −6.197 | −7.389 | 1.00 | 88.63 | E |
| ATOM | 17413 | CB | PHE | E | 374 | 82.821 | −7.636 | −6.905 | 1.00 | 83.52 | E |
| ATOM | 17414 | CG | PHE | E | 374 | 82.719 | −8.616 | −8.019 | 1.00 | 63.70 | E |
| ATOM | 17415 | CD1 | PHE | E | 374 | 83.861 | −9.083 | −8.657 | 1.00 | 63.70 | E |
| ATOM | 17416 | CD2 | PHE | E | 374 | 81.473 | −9.026 | −8.479 | 1.00 | 63.70 | E |
| ATOM | 17417 | CE1 | PHE | E | 374 | 83.766 | −9.945 | −9.739 | 1.00 | 63.70 | E |
| ATOM | 17418 | CE2 | PHE | E | 374 | 81.368 | −9.884 | −9.559 | 1.00 | 63.70 | E |
| ATOM | 17419 | CZ | PHE | E | 374 | 82.518 | −10.345 | −10.191 | 1.00 | 63.70 | E |
| ATOM | 17420 | C | PHE | E | 374 | 82.637 | −5.224 | −6.274 | 1.00 | 84.71 | E |
| ATOM | 17421 | O | PHE | E | 374 | 81.638 | −5.375 | −5.573 | 1.00 | 82.95 | E |
| ATOM | 17422 | N | LYS | E | 375 | 83.490 | −4.214 | −6.133 | 1.00 | 63.61 | E |
| ATOM | 17423 | CA | LYS | E | 375 | 83.333 | −3.187 | −5.111 | 1.00 | 66.24 | E |
| ATOM | 17424 | CB | LYS | E | 375 | 82.655 | −1.951 | −5.710 | 1.00 | 126.58 | E |
| ATOM | 17425 | CG | LYS | E | 375 | 83.572 | −1.027 | −6.512 | 1.00 | 100.42 | E |
| ATOM | 17426 | CD | LYS | E | 375 | 84.027 | −1.635 | −7.830 | 1.00 | 100.42 | E |
| ATOM | 17427 | CE | LYS | E | 375 | 84.864 | −0.640 | −8.634 | 1.00 | 100.42 | E |
| ATOM | 17428 | NZ | LYS | E | 375 | 85.371 | −1.215 | −9.917 | 1.00 | 100.42 | E |
| ATOM | 17429 | C | LYS | E | 375 | 82.560 | −3.630 | −3.870 | 1.00 | 71.26 | E |
| ATOM | 17430 | O | LYS | E | 375 | 81.567 | −3.012 | −3.507 | 1.00 | 73.52 | E |
| ATOM | 17431 | N | GLY | E | 376 | 83.002 | −4.703 | −3.227 | 1.00 | 75.37 | E |
| ATOM | 17432 | CA | GLY | E | 376 | 82.318 | −5.146 | −2.028 | 1.00 | 75.37 | E |
| ATOM | 17433 | C | GLY | E | 376 | 81.093 | −6.032 | −2.179 | 1.00 | 75.37 | E |
| ATOM | 17434 | O | GLY | E | 376 | 80.819 | −6.839 | −1.286 | 1.00 | 75.37 | E |
| ATOM | 17435 | N | GLU | E | 377 | 80.339 | −5.882 | −3.269 | 1.00 | 66.68 | E |
| ATOM | 17436 | CA | GLU | E | 377 | 79.156 | −6.718 | −3.493 | 1.00 | 66.68 | E |
| ATOM | 17437 | CB | GLU | E | 377 | 77.987 | −5.876 | −3.991 | 1.00 | 105.61 | E |
| ATOM | 17438 | CG | GLU | E | 377 | 76.667 | −6.631 | −3.995 | 1.00 | 105.61 | E |
| ATOM | 17439 | CD | GLU | E | 377 | 75.500 | −5.766 | −4.433 | 1.00 | 105.61 | E |
| ATOM | 17440 | OE1 | GLU | E | 377 | 74.337 | −6.218 | −4.311 | 1.00 | 105.61 | E |
| ATOM | 17441 | OE2 | GLU | E | 377 | 75.748 | −4.635 | −4.902 | 1.00 | 105.61 | E |
| ATOM | 17442 | C | GLU | E | 377 | 79.463 | −7.819 | −4.512 | 1.00 | 66.68 | E |
| ATOM | 17443 | O | GLU | E | 377 | 79.747 | −7.531 | −5.675 | 1.00 | 66.68 | E |
| ATOM | 17444 | N | TYR | E | 378 | 79.402 | −9.078 | −4.074 | 1.00 | 76.81 | E |
| ATOM | 17445 | CA | TYR | E | 378 | 79.709 | −10.197 | −4.956 | 1.00 | 76.81 | E |
| ATOM | 17446 | CB | TYR | E | 378 | 80.405 | −11.309 | −4.171 | 1.00 | 84.86 | E |
| ATOM | 17447 | CG | TYR | E | 378 | 81.786 | −10.906 | −3.735 | 1.00 | 84.86 | E |
| ATOM | 17448 | CD1 | TYR | E | 378 | 82.009 | −10.351 | −2.476 | 1.00 | 84.86 | E |
| ATOM | 17449 | CE1 | TYR | E | 378 | 83.274 | −9.902 | −2.101 | 1.00 | 84.86 | E |
| ATOM | 17450 | CD2 | TYR | E | 378 | 82.861 | −11.007 | −4.609 | 1.00 | 84.86 | E |
| ATOM | 17451 | CE2 | TYR | E | 378 | 84.124 | −10.561 | −4.251 | 1.00 | 84.86 | E |
| ATOM | 17452 | CZ | TYR | E | 378 | 84.326 | −10.009 | −2.996 | 1.00 | 84.86 | E |
| ATOM | 17453 | OH | TYR | E | 378 | 85.582 | −9.562 | −2.643 | 1.00 | 84.86 | E |
| ATOM | 17454 | C | TYR | E | 378 | 78.540 | −10.757 | −5.760 | 1.00 | 76.81 | E |
| ATOM | 17455 | O | TYR | E | 378 | 78.103 | −11.896 | −5.571 | 1.00 | 76.81 | E |
| ATOM | 17456 | N | LYS | E | 379 | 78.044 | −9.921 | −6.663 | 1.00 | 51.05 | E |
| ATOM | 17457 | CA | LYS | E | 379 | 76.954 | −10.263 | −7.573 | 1.00 | 51.05 | E |
| ATOM | 17458 | CB | LYS | E | 379 | 75.660 | −9.556 | −7.184 | 1.00 | 89.00 | E |
| ATOM | 17459 | CG | LYS | E | 379 | 75.069 | −10.017 | −5.869 | 1.00 | 89.00 | E |
| ATOM | 17460 | CD | LYS | E | 379 | 73.795 | −9.242 | −5.559 | 1.00 | 89.00 | E |
| ATOM | 17461 | CE | LYS | E | 379 | 73.260 | −9.541 | −4.160 | 1.00 | 89.00 | E |
| ATOM | 17462 | NZ | LYS | E | 379 | 72.117 | −8.643 | −3.818 | 1.00 | 89.00 | E |
| ATOM | 17463 | C | LYS | E | 379 | 77.407 | −9.775 | −8.941 | 1.00 | 51.05 | E |
| ATOM | 17464 | O | LYS | E | 379 | 77.998 | −8.703 | −9.059 | 1.00 | 51.05 | E |
| ATOM | 17465 | N | TYR | E | 380 | 77.166 | −10.578 | −9.970 | 1.00 | 43.71 | E |
| ATOM | 17466 | CA | TYR | E | 380 | 77.558 | −10.220 | −11.332 | 1.00 | 43.71 | E |
| ATOM | 17467 | CB | TYR | E | 380 | 77.842 | −11.481 | −12.138 | 1.00 | 46.60 | E |
| ATOM | 17468 | CG | TYR | E | 380 | 78.359 | −11.210 | −13.520 | 1.00 | 46.60 | E |
| ATOM | 17469 | CD1 | TYR | E | 380 | 79.724 | −11.027 | −13.754 | 1.00 | 46.60 | E |
| ATOM | 17470 | CE1 | TYR | E | 380 | 80.208 | −10.782 | −15.036 | 1.00 | 46.60 | E |
| ATOM | 17471 | CD2 | TYR | E | 380 | 77.485 | −11.138 | −14.604 | 1.00 | 46.60 | E |
| ATOM | 17472 | CE2 | TYR | E | 380 | 77.957 | −10.890 | −15.887 | 1.00 | 46.60 | E |
| ATOM | 17473 | CZ | TYR | E | 380 | 79.318 | −10.714 | −16.097 | 1.00 | 46.60 | E |
| ATOM | 17474 | OH | TYR | E | 380 | 79.784 | −10.479 | −17.371 | 1.00 | 46.60 | E |
| ATOM | 17475 | C | TYR | E | 380 | 76.384 | −9.469 | −11.940 | 1.00 | 43.71 | E |
| ATOM | 17476 | O | TYR | E | 380 | 75.333 | −10.058 | −12.189 | 1.00 | 43.71 | E |
| ATOM | 17477 | N | VAL | E | 381 | 76.555 | −8.172 | −12.159 | 1.00 | 52.04 | E |
| ATOM | 17478 | CA | VAL | E | 381 | 75.483 | −7.349 | −12.710 | 1.00 | 52.04 | E |
| ATOM | 17479 | CB | VAL | E | 381 | 75.684 | −5.869 | −12.352 | 1.00 | 61.67 | E |
| ATOM | 17480 | CG1 | VAL | E | 381 | 74.509 | −5.052 | −12.843 | 1.00 | 61.67 | E |
| ATOM | 17481 | CG2 | VAL | E | 381 | 75.840 | −5.728 | −10.857 | 1.00 | 61.67 | E |
| ATOM | 17482 | C | VAL | E | 381 | 75.421 | −7.494 | −14.222 | 1.00 | 52.04 | E |
| ATOM | 17483 | O | VAL | E | 381 | 76.392 | −7.188 | −14.922 | 1.00 | 52.04 | E |
| ATOM | 17484 | N | ASN | E | 382 | 74.268 | −7.932 | −14.718 | 1.00 | 83.06 | E |
| ATOM | 17485 | CA | ASN | E | 382 | 74.084 | −8.168 | −16.141 | 1.00 | 83.06 | E |
| ATOM | 17486 | CB | ASN | E | 382 | 72.611 | −8.192 | −16.496 | 1.00 | 48.50 | E |
| ATOM | 17487 | CG | ASN | E | 382 | 72.337 | −9.031 | −17.739 | 1.00 | 48.50 | E |
| ATOM | 17488 | OD1 | ASN | E | 382 | 73.171 | −9.118 | −18.650 | 1.00 | 48.50 | E |
| ATOM | 17489 | ND2 | ASN | E | 382 | 71.161 | −9.648 | −17.784 | 1.00 | 48.50 | E |
| ATOM | 17490 | C | ASN | E | 382 | 74.788 | −7.221 | −17.101 | 1.00 | 83.06 | E |
| ATOM | 17491 | O | ASN | E | 382 | 75.858 | −7.544 | −17.638 | 1.00 | 83.06 | E |
| ATOM | 17492 | N | GLY | E | 383 | 74.200 | −6.056 | −17.339 | 1.00 | 43.58 | E |
| ATOM | 17493 | CA | GLY | E | 383 | 74.835 | −5.142 | −18.278 | 1.00 | 43.58 | E |
| ATOM | 17494 | C | GLY | E | 383 | 76.160 | −4.485 | −17.898 | 1.00 | 43.58 | E |
| ATOM | 17495 | O | GLY | E | 383 | 76.702 | −3.713 | −18.686 | 1.00 | 43.58 | E |
| ATOM | 17496 | N | ARG | E | 384 | 76.698 | −4.779 | −16.719 | 1.00 | 50.80 | E |
| ATOM | 17497 | CA | ARG | E | 384 | 77.939 | −4.143 | −16.292 | 1.00 | 50.80 | E |
| ATOM | 17498 | CB | ARG | E | 384 | 78.090 | −4.287 | −14.780 | 1.00 | 54.51 | E |
| ATOM | 17499 | CG | ARG | E | 384 | 79.240 | −3.501 | −14.179 | 1.00 | 54.51 | E |
| ATOM | 17500 | CD | ARG | E | 384 | 79.059 | −3.408 | −12.677 | 1.00 | 54.51 | E |
| ATOM | 17501 | NE | ARG | E | 384 | 77.788 | −2.770 | −12.348 | 1.00 | 54.51 | E |
| ATOM | 17502 | CZ | ARG | E | 384 | 77.270 | −2.702 | −11.126 | 1.00 | 54.51 | E |
| ATOM | 17503 | NH1 | ARG | E | 384 | 77.916 | −3.237 | −10.096 | 1.00 | 54.51 | E |
| ATOM | 17504 | NH2 | ARG | E | 384 | 76.101 | −2.102 | −10.930 | 1.00 | 54.51 | E |
| ATOM | 17505 | C | ARG | E | 384 | 79.185 | −4.664 | −16.987 | 1.00 | 50.80 | E |
| ATOM | 17506 | O | ARG | E | 384 | 79.253 | −5.828 | −17.370 | 1.00 | 50.80 | E |
| ATOM | 17507 | N | TYR | E | 385 | 80.169 | −3.788 | −17.150 | 1.00 | 55.11 | E |
| ATOM | 17508 | CA | TYR | E | 385 | 81.422 | −4.163 | −17.783 | 1.00 | 55.11 | E |
| ATOM | 17509 | CB | TYR | E | 385 | 82.067 | −2.944 | −18.428 | 1.00 | 67.90 | E |
| ATOM | 17510 | CG | TYR | E | 385 | 83.456 | −3.230 | −18.915 | 1.00 | 67.90 | E |
| ATOM | 17511 | CD1 | TYR | E | 385 | 83.666 | −3.979 | −20.066 | 1.00 | 67.90 | E |
| ATOM | 17512 | CE1 | TYR | E | 385 | 84.949 | −4.318 | −20.474 | 1.00 | 67.90 | E |
| ATOM | 17513 | CD2 | TYR | E | 385 | 84.566 | −2.820 | −18.181 | 1.00 | 67.90 | E |
| ATOM | 17514 | CE2 | TYR | E | 385 | 85.856 | −3.154 | −18.577 | 1.00 | 67.90 | E |
| ATOM | 17515 | CZ | TYR | E | 385 | 86.042 | −3.905 | −19.726 | 1.00 | 67.90 | E |
| ATOM | 17516 | OH | TYR | E | 385 | 87.320 | −4.245 | −20.123 | 1.00 | 67.90 | E |
| ATOM | 17517 | C | TYR | E | 385 | 82.389 | −4.745 | −16.746 | 1.00 | 55.11 | E |
| ATOM | 17518 | O | TYR | E | 385 | 82.434 | −4.274 | −15.609 | 1.00 | 55.11 | E |
| ATOM | 17519 | N | TYR | E | 386 | 83.161 | −5.761 | −17.139 | 1.00 | 59.24 | E |
| ATOM | 17520 | CA | TYR | E | 386 | 84.138 | −6.378 | −16.242 | 1.00 | 59.24 | E |
| ATOM | 17521 | CB | TYR | E | 386 | 83.574 | −7.620 | −15.548 | 1.00 | 52.22 | E |
| ATOM | 17522 | CG | TYR | E | 386 | 82.403 | −7.413 | −14.618 | 1.00 | 52.22 | E |
| ATOM | 17523 | CD1 | TYR | E | 386 | 81.099 | −7.622 | −15.054 | 1.00 | 52.22 | E |
| ATOM | 17524 | CE1 | TYR | E | 386 | 80.016 | −7.479 | −14.190 | 1.00 | 52.22 | E |
| ATOM | 17525 | CD2 | TYR | E | 386 | 82.601 | −7.045 | −13.287 | 1.00 | 52.22 | E |
| ATOM | 17526 | CE2 | TYR | E | 386 | 81.522 | −6.894 | −12.411 | 1.00 | 52.22 | E |
| ATOM | 17527 | CZ | TYR | E | 386 | 80.232 | −7.115 | −12.873 | 1.00 | 52.22 | E |
| ATOM | 17528 | OH | TYR | E | 386 | 79.157 | −6.972 | −12.025 | 1.00 | 52.22 | E |
| ATOM | 17529 | C | TYR | E | 386 | 85.383 | −6.853 | −16.961 | 1.00 | 59.24 | E |
| ATOM | 17530 | O | TYR | E | 386 | 85.282 | −7.636 | −17.897 | 1.00 | 59.24 | E |
| ATOM | 17531 | N | PRO | E | 387 | 86.573 | −6.386 | −16.553 | 1.00 | 57.16 | E |
| ATOM | 17532 | CD | PRO | E | 387 | 86.866 | −5.271 | −15.639 | 1.00 | 67.07 | E |
| ATOM | 17533 | CA | PRO | E | 387 | 87.799 | −6.857 | −17.220 | 1.00 | 57.16 | E |
| ATOM | 17534 | CB | PRO | E | 387 | 88.891 | −6.059 | −16.520 | 1.00 | 67.07 | E |
| ATOM | 17535 | CG | PRO | E | 387 | 88.189 | −4.784 | −16.161 | 1.00 | 67.07 | E |
| ATOM | 17536 | C | PRO | E | 387 | 87.817 | −8.344 | −16.825 | 1.00 | 57.16 | E |
| ATOM | 17537 | O | PRO | E | 387 | 87.383 | −8.665 | −15.717 | 1.00 | 57.16 | E |
| ATOM | 17538 | N | GLU | E | 388 | 88.306 | −9.259 | −17.660 | 1.00 | 92.02 | E |
| ATOM | 17539 | CA | GLU | E | 388 | 88.188 | −10.639 | −17.214 | 1.00 | 92.02 | E |
| ATOM | 17540 | CB | GLU | E | 388 | 86.918 | −11.236 | −17.816 | 1.00 | 101.85 | E |
| ATOM | 17541 | CG | GLU | E | 388 | 86.985 | −11.453 | −19.308 | 1.00 | 101.85 | E |
| ATOM | 17542 | CD | GLU | E | 388 | 85.838 | −12.302 | −19.810 | 1.00 | 101.85 | E |
| ATOM | 17543 | OE1 | GLU | E | 388 | 85.684 | −13.440 | −19.323 | 1.00 | 101.85 | E |
| ATOM | 17544 | OE2 | GLU | E | 388 | 85.089 | −11.833 | −20.689 | 1.00 | 101.85 | E |
| ATOM | 17545 | C | GLU | E | 388 | 89.269 | −11.713 | −17.279 | 1.00 | 92.02 | E |
| ATOM | 17546 | O | GLU | E | 388 | 88.932 | −12.894 | −17.217 | 1.00 | 92.02 | E |
| ATOM | 17547 | N | TRP | E | 389 | 90.545 | −11.372 | −17.380 | 1.00 | 102.91 | E |
| ATOM | 17548 | CA | TRP | E | 389 | 91.546 | −12.445 | −17.397 | 1.00 | 102.91 | E |
| ATOM | 17549 | CB | TRP | E | 389 | 91.606 | −13.130 | −16.025 | 1.00 | 115.38 | E |
| ATOM | 17550 | CG | TRP | E | 389 | 92.679 | −12.662 | −15.103 | 1.00 | 115.38 | E |
| ATOM | 17551 | CD2 | TRP | E | 389 | 93.759 | −11.763 | −15.403 | 1.00 | 115.38 | E |
| ATOM | 17552 | CE2 | TRP | E | 389 | 94.537 | −11.642 | −14.232 | 1.00 | 115.38 | E |
| ATOM | 17553 | CE3 | TRP | E | 389 | 94.146 | −11.048 | −16.549 | 1.00 | 115.38 | E |
| ATOM | 17554 | CD1 | TRP | E | 389 | 92.844 | −13.036 | −13.803 | 1.00 | 115.38 | E |
| ATOM | 17555 | NE1 | TRP | E | 389 | 93.955 | −12.429 | −13.272 | 1.00 | 115.38 | E |
| ATOM | 17556 | CZ2 | TRP | E | 389 | 95.682 | −10.835 | −14.171 | 1.00 | 115.38 | E |
| ATOM | 17557 | CZ3 | TRP | E | 389 | 95.286 | −10.246 | −16.487 | 1.00 | 115.38 | E |
| ATOM | 17558 | CH2 | TRP | E | 389 | 96.039 | −10.148 | −15.305 | 1.00 | 115.38 | E |
| ATOM | 17559 | C | TRP | E | 389 | 91.222 | −13.518 | −18.437 | 1.00 | 102.91 | E |
| ATOM | 17560 | O | TRP | E | 389 | 90.831 | −14.628 | −18.079 | 1.00 | 102.91 | E |
| ATOM | 17561 | N | GLY | E | 390 | 91.391 | −13.198 | −19.714 | 1.00 | 86.11 | E |
| ATOM | 17562 | CA | GLY | E | 390 | 91.098 | −14.167 | −20.756 | 1.00 | 86.11 | E |
| ATOM | 17563 | C | GLY | E | 390 | 91.983 | −15.402 | −20.737 | 1.00 | 86.11 | E |
| ATOM | 17564 | O | GLY | E | 390 | 93.107 | −15.368 | −20.237 | 1.00 | 86.11 | E |
| ATOM | 17565 | N | ARG | E | 391 | 91.460 | −16.501 | −21.274 | 1.00 | 98.53 | E |
| ATOM | 17566 | CA | ARG | E | 391 | 92.199 | −17.758 | −21.349 | 1.00 | 98.53 | E |
| ATOM | 17567 | CB | ARG | E | 391 | 91.229 | −18.943 | −21.246 | 1.00 | 97.07 | E |
| ATOM | 17568 | CG | ARG | E | 391 | 90.398 | −18.979 | −19.954 | 1.00 | 97.07 | E |
| ATOM | 17569 | CD | ARG | E | 391 | 91.008 | −19.880 | −18.863 | 1.00 | 97.07 | E |
| ATOM | 17570 | NE | ARG | E | 391 | 90.525 | −21.265 | −18.913 | 1.00 | 97.07 | E |
| ATOM | 17571 | CZ | ARG | E | 391 | 90.940 | −22.189 | −19.783 | 1.00 | 97.07 | E |
| ATOM | 17572 | NH1 | ARG | E | 391 | 91.863 | −21.892 | −20.692 | 1.00 | 97.07 | E |
| ATOM | 17573 | NH2 | ARG | E | 391 | 90.423 | −23.412 | −19.753 | 1.00 | 97.07 | E |
| ATOM | 17574 | C | ARG | E | 391 | 92.906 | −17.760 | −22.707 | 1.00 | 98.53 | E |
| ATOM | 17575 | O | ARG | E | 391 | 92.601 | −16.933 | −23.563 | 1.00 | 98.53 | E |
| ATOM | 17576 | N | LYS | E | 392 | 93.855 | −18.668 | −22.905 | 1.00 | 76.10 | E |
| ATOM | 17577 | CA | LYS | E | 392 | 94.566 | −18.732 | −24.179 | 1.00 | 76.10 | E |
| ATOM | 17578 | CB | LYS | E | 392 | 96.079 | −18.714 | −23.944 | 1.00 | 107.64 | E |
| ATOM | 17579 | CG | LYS | E | 392 | 96.888 | −18.762 | −25.232 | 1.00 | 107.64 | E |
| ATOM | 17580 | CD | LYS | E | 392 | 98.366 | −18.980 | −24.966 | 1.00 | 107.64 | E |
| ATOM | 17581 | CE | LYS | E | 392 | 99.126 | −19.193 | −26.272 | 1.00 | 107.64 | E |
| ATOM | 17582 | NZ | LYS | E | 392 | 100.546 | −19.583 | −26.035 | 1.00 | 107.64 | E |
| ATOM | 17583 | C | LYS | E | 392 | 94.184 | −19.986 | −24.980 | 1.00 | 76.10 | E |
| ATOM | 17584 | O | LYS | E | 392 | 94.456 | −21.113 | −24.558 | 1.00 | 76.10 | E |
| ATOM | 17585 | N | SER | E | 393 | 93.554 | −19.778 | −26.137 | 1.00 | 70.08 | E |
| ATOM | 17586 | CA | SER | E | 393 | 93.125 | −20.877 | −26.999 | 1.00 | 70.08 | E |
| ATOM | 17587 | CB | SER | E | 393 | 91.880 | −21.553 | −26.416 | 1.00 | 73.14 | E |
| ATOM | 17588 | OG | SER | E | 393 | 91.403 | −22.575 | −27.278 | 1.00 | 73.14 | E |
| ATOM | 17589 | C | SER | E | 393 | 92.801 | −20.353 | −28.386 | 1.00 | 70.08 | E |
| ATOM | 17590 | O | SER | E | 393 | 92.531 | −19.167 | −28.550 | 1.00 | 70.08 | E |
| ATOM | 17591 | N | ASN | E | 394 | 92.834 | −21.230 | −29.385 | 1.00 | 58.81 | E |
| ATOM | 17592 | CA | ASN | E | 394 | 92.509 | −20.814 | −30.743 | 1.00 | 58.81 | E |
| ATOM | 17593 | CB | ASN | E | 394 | 93.012 | −21.826 | −31.759 | 1.00 | 71.26 | E |
| ATOM | 17594 | CG | ASN | E | 394 | 94.510 | −21.813 | −31.888 | 1.00 | 71.26 | E |
| ATOM | 17595 | OD1 | ASN | E | 394 | 95.125 | −20.753 | −32.051 | 1.00 | 71.26 | E |
| ATOM | 17596 | ND2 | ASN | E | 394 | 95.113 | −22.996 | −31.829 | 1.00 | 71.26 | E |
| ATOM | 17597 | C | ASN | E | 394 | 91.005 | −20.667 | −30.876 | 1.00 | 58.81 | E |
| ATOM | 17598 | O | ASN | E | 394 | 90.502 | −20.025 | −31.797 | 1.00 | 58.81 | E |
| ATOM | 17599 | N | TYR | E | 395 | 90.298 | −21.274 | −29.937 | 1.00 | 46.76 | E |
| ATOM | 17600 | CA | TYR | E | 395 | 88.855 | −21.218 | −29.907 | 1.00 | 46.76 | E |
| ATOM | 17601 | CB | TYR | E | 395 | 88.291 | −22.564 | −29.461 | 1.00 | 50.72 | E |
| ATOM | 17602 | CG | TYR | E | 395 | 88.562 | −23.695 | −30.412 | 1.00 | 50.72 | E |
| ATOM | 17603 | CD1 | TYR | E | 395 | 89.647 | −24.539 | −30.224 | 1.00 | 50.72 | E |
| ATOM | 17604 | CE1 | TYR | E | 395 | 89.915 | −25.579 | −31.108 | 1.00 | 50.72 | E |
| ATOM | 17605 | CD2 | TYR | E | 395 | 87.737 | −23.916 | −31.518 | 1.00 | 50.72 | E |
| ATOM | 17606 | CE2 | TYR | E | 395 | 87.994 | −24.951 | −32.411 | 1.00 | 50.72 | E |
| ATOM | 17607 | CZ | TYR | E | 395 | 89.089 | −25.780 | −32.199 | 1.00 | 50.72 | E |
| ATOM | 17608 | OH | TYR | E | 395 | 89.361 | −26.804 | −33.086 | 1.00 | 50.72 | E |
| ATOM | 17609 | C | TYR | E | 395 | 88.413 | −20.148 | −28.921 | 1.00 | 46.76 | E |
| ATOM | 17610 | O | TYR | E | 395 | 89.149 | −19.820 | −27.992 | 1.00 | 46.76 | E |
| ATOM | 17611 | N | GLU | E | 396 | 87.213 | −19.606 | −29.124 | 1.00 | 52.00 | E |
| ATOM | 17612 | CA | GLU | E | 396 | 86.654 | −18.598 | −28.228 | 1.00 | 52.00 | E |
| ATOM | 17613 | CB | GLU | E | 396 | 85.956 | −17.498 | −29.025 | 1.00 | 105.94 | E |
| ATOM | 17614 | CG | GLU | E | 396 | 86.772 | −16.933 | −30.162 | 1.00 | 105.94 | E |
| ATOM | 17615 | CD | GLU | E | 396 | 85.947 | −16.048 | −31.072 | 1.00 | 105.94 | E |
| ATOM | 17616 | OE1 | GLU | E | 396 | 84.881 | −16.510 | −31.540 | 1.00 | 105.94 | E |
| ATOM | 17617 | OE2 | GLU | E | 396 | 86.360 | −14.898 | −31.327 | 1.00 | 105.94 | E |
| ATOM | 17618 | C | GLU | E | 396 | 85.624 | −19.333 | −27.371 | 1.00 | 52.00 | E |
| ATOM | 17619 | O | GLU | E | 396 | 84.912 | −20.206 | −27.870 | 1.00 | 52.00 | E |
| ATOM | 17620 | N | PRO | E | 397 | 85.546 | −19.016 | −26.068 | 1.00 | 45.74 | E |
| ATOM | 17621 | CD | PRO | E | 397 | 86.251 | −17.986 | −25.296 | 1.00 | 41.29 | E |
| ATOM | 17622 | CA | PRO | E | 397 | 84.557 | −19.708 | −25.235 | 1.00 | 45.74 | E |
| ATOM | 17623 | CB | PRO | E | 397 | 84.652 | −18.980 | −23.888 | 1.00 | 41.29 | E |
| ATOM | 17624 | CG | PRO | E | 397 | 85.225 | −17.640 | −24.251 | 1.00 | 41.29 | E |
| ATOM | 17625 | C | PRO | E | 397 | 83.183 | −19.604 | −25.885 | 1.00 | 45.74 | E |
| ATOM | 17626 | O | PRO | E | 397 | 82.788 | −18.534 | −26.359 | 1.00 | 45.74 | E |
| ATOM | 17627 | N | GLY | E | 398 | 82.475 | −20.730 | −25.924 | 1.00 | 44.55 | E |
| ATOM | 17628 | CA | GLY | E | 398 | 81.172 | −20.772 | −26.559 | 1.00 | 44.55 | E |
| ATOM | 17629 | C | GLY | E | 398 | 81.244 | −21.747 | −27.719 | 1.00 | 44.55 | E |
| ATOM | 17630 | O | GLY | E | 398 | 80.313 | −22.513 | −27.944 | 1.00 | 44.55 | E |
| ATOM | 17631 | N | GLU | E | 399 | 82.350 | −21.715 | −28.460 | 1.00 | 41.08 | E |
| ATOM | 17632 | CA | GLU | E | 399 | 82.556 | −22.628 | −29.593 | 1.00 | 41.08 | E |
| ATOM | 17633 | CB | GLU | E | 399 | 83.781 | −22.202 | −30.430 | 1.00 | 47.90 | E |
| ATOM | 17634 | CG | GLU | E | 399 | 83.745 | −20.762 | −30.905 | 1.00 | 47.90 | E |
| ATOM | 17635 | CD | GLU | E | 399 | 84.936 | −20.365 | −31.772 | 1.00 | 47.90 | E |
| ATOM | 17636 | OE1 | GLU | E | 399 | 86.076 | −20.732 | −31.437 | 1.00 | 47.90 | E |
| ATOM | 17637 | OE2 | GLU | E | 399 | 84.740 | −19.659 | −32.786 | 1.00 | 47.90 | E |
| ATOM | 17638 | C | GLU | E | 399 | 82.810 | −24.012 | −28.989 | 1.00 | 41.08 | E |
| ATOM | 17639 | O | GLU | E | 399 | 83.540 | −24.142 | −28.011 | 1.00 | 41.08 | E |
| ATOM | 17640 | N | PRO | E | 400 | 82.205 | −25.065 | −29.545 | 1.00 | 61.72 | E |
| ATOM | 17641 | CD | PRO | E | 400 | 81.244 | −25.177 | −30.650 | 1.00 | 44.24 | E |
| ATOM | 17642 | CA | PRO | E | 400 | 82.467 | −26.375 | −28.943 | 1.00 | 56.80 | E |
| ATOM | 17643 | CB | PRO | E | 400 | 81.652 | −27.340 | −29.821 | 1.00 | 92.85 | E |
| ATOM | 17644 | CG | PRO | E | 400 | 81.443 | −26.609 | −31.089 | 1.00 | 44.24 | E |
| ATOM | 17645 | C | PRO | E | 400 | 83.946 | −26.756 | −28.837 | 1.00 | 47.55 | E |
| ATOM | 17646 | O | PRO | E | 400 | 84.319 | −27.602 | −28.033 | 1.00 | 47.76 | E |
| ATOM | 17647 | N | GLY | E | 401 | 84.794 | −26.105 | −29.621 | 1.00 | 50.80 | E |
| ATOM | 17648 | CA | GLY | E | 401 | 86.209 | −26.421 | −29.575 | 1.00 | 50.36 | E |
| ATOM | 17649 | C | GLY | E | 401 | 86.959 | −25.889 | −28.365 | 1.00 | 50.15 | E |
| ATOM | 17650 | O | GLY | E | 401 | 88.125 | −26.225 | −28.169 | 1.00 | 50.86 | E |
| ATOM | 17651 | N | PHE | E | 402 | 86.315 | −25.056 | −27.551 | 1.00 | 58.01 | E |
| ATOM | 17652 | CA | PHE | E | 402 | 86.983 | −24.509 | −26.376 | 1.00 | 58.01 | E |
| ATOM | 17653 | CB | PHE | E | 402 | 86.157 | −23.389 | −25.754 | 1.00 | 156.51 | E |
| ATOM | 17654 | CG | PHE | E | 402 | 86.965 | −22.482 | −24.894 | 1.00 | 54.84 | E |
| ATOM | 17655 | CD1 | PHE | E | 402 | 87.811 | −21.534 | −25.473 | 1.00 | 54.84 | E |
| ATOM | 17656 | CD2 | PHE | E | 402 | 86.947 | −22.617 | −23.516 | 1.00 | 54.84 | E |
| ATOM | 17657 | CE1 | PHE | E | 402 | 88.632 | −20.738 | −24.694 | 1.00 | 54.84 | E |
| ATOM | 17658 | CE2 | PHE | E | 402 | 87.761 | −21.829 | −22.722 | 1.00 | 54.84 | E |
| ATOM | 17659 | CZ | PHE | E | 402 | 88.611 | −20.883 | −23.314 | 1.00 | 54.84 | E |
| ATOM | 17660 | C | PHE | E | 402 | 87.230 | −25.602 | −25.332 | 1.00 | 58.01 | E |
| ATOM | 17661 | O | PHE | E | 402 | 86.312 | −26.330 | −24.956 | 1.00 | 58.01 | E |
| ATOM | 17662 | N | PRO | E | 403 | 88.477 | −25.722 | −24.842 | 1.00 | 78.16 | E |
| ATOM | 17663 | CD | PRO | E | 403 | 89.612 | −24.874 | −25.224 | 1.00 | 51.43 | E |
| ATOM | 17664 | CA | PRO | E | 403 | 88.910 | −26.708 | −23.847 | 1.00 | 78.16 | E |
| ATOM | 17665 | CB | PRO | E | 403 | 90.427 | −26.544 | −23.819 | 1.00 | 156.26 | E |
| ATOM | 17666 | CG | PRO | E | 403 | 90.742 | −25.823 | −25.070 | 1.00 | 51.43 | E |
| ATOM | 17667 | C | PRO | E | 403 | 88.334 | −26.495 | −22.462 | 1.00 | 78.16 | E |
| ATOM | 17668 | O | PRO | E | 403 | 88.799 | −25.640 | −21.721 | 1.00 | 78.16 | E |
| ATOM | 17669 | N | TRP | E | 404 | 87.315 | −27.258 | −22.112 | 1.00 | 55.96 | E |
| ATOM | 17670 | CA | TRP | E | 404 | 86.735 | −27.155 | −20.786 | 1.00 | 55.96 | E |
| ATOM | 17671 | CB | TRP | E | 404 | 85.235 | −26.830 | −20.838 | 1.00 | 67.11 | E |
| ATOM | 17672 | CG | TRP | E | 404 | 84.893 | −25.404 | −21.171 | 1.00 | 60.35 | E |
| ATOM | 17673 | CD2 | TRP | E | 404 | 85.237 | −24.232 | −20.420 | 1.00 | 60.35 | E |
| ATOM | 17674 | CE2 | TRP | E | 404 | 84.679 | −23.126 | −21.094 | 1.00 | 60.35 | E |
| ATOM | 17675 | CE3 | TRP | E | 404 | 85.962 | −24.007 | −19.240 | 1.00 | 60.35 | E |
| ATOM | 17676 | CD1 | TRP | E | 404 | 84.164 | −24.970 | −22.238 | 1.00 | 60.35 | E |
| ATOM | 17677 | NE1 | TRP | E | 404 | 84.030 | −23.608 | −22.201 | 1.00 | 60.35 | E |
| ATOM | 17678 | CZ2 | TRP | E | 404 | 84.824 | −21.807 | −20.629 | 1.00 | 60.35 | E |
| ATOM | 17679 | CZ3 | TRP | E | 404 | 86.105 | −22.689 | −18.778 | 1.00 | 60.35 | E |
| ATOM | 17680 | CH2 | TRP | E | 404 | 85.538 | −21.613 | −19.474 | 1.00 | 60.35 | E |
| ATOM | 17681 | C | TRP | E | 404 | 86.928 | −28.536 | −20.191 | 1.00 | 55.96 | E |
| ATOM | 17682 | O | TRP | E | 404 | 86.622 | −29.534 | −20.836 | 1.00 | 55.96 | E |
| ATOM | 17683 | N | ASN | E | 405 | 87.439 | −28.605 | −18.969 | 1.00 | 52.92 | E |
| ATOM | 17684 | CA | ASN | E | 405 | 87.646 | −29.895 | −18.334 | 1.00 | 52.95 | E |
| ATOM | 17685 | CB | ASN | E | 405 | 88.666 | −29.765 | −17.213 | 1.00 | 89.79 | E |
| ATOM | 17686 | CG | ASN | E | 405 | 89.314 | −31.071 | −16.885 | 1.00 | 89.79 | E |
| ATOM | 17687 | OD1 | ASN | E | 405 | 88.699 | −31.956 | −16.294 | 1.00 | 89.79 | E |
| ATOM | 17688 | ND2 | ASN | E | 405 | 90.565 | −31.214 | −17.287 | 1.00 | 89.79 | E |
| ATOM | 17689 | C | ASN | E | 405 | 86.329 | −30.434 | −17.788 | 1.00 | 61.89 | E |
| ATOM | 17690 | O | ASN | E | 405 | 86.082 | −30.410 | −16.578 | 1.00 | 54.95 | E |
| ATOM | 17691 | N | ILE | E | 406 | 85.482 | −30.911 | −18.692 | 1.00 | 54.26 | E |
| ATOM | 17692 | CA | ILE | E | 406 | 84.194 | −31.448 | −18.304 | 1.00 | 46.46 | E |
| ATOM | 17693 | CB | ILE | E | 406 | 83.054 | −30.828 | −19.134 | 1.00 | 68.01 | E |
| ATOM | 17694 | CG2 | ILE | E | 406 | 81.701 | −31.318 | −18.621 | 1.00 | 55.63 | E |
| ATOM | 17695 | CG1 | ILE | E | 406 | 83.122 | −29.311 | −19.041 | 1.00 | 55.63 | E |
| ATOM | 17696 | CD1 | ILE | E | 406 | 82.006 | −28.601 | −19.777 | 1.00 | 55.63 | E |
| ATOM | 17697 | C | ILE | E | 406 | 84.186 | −32.941 | −18.530 | 1.00 | 53.33 | E |
| ATOM | 17698 | O | ILE | E | 406 | 84.762 | −33.414 | −19.508 | 1.00 | 48.43 | E |
| ATOM | 17699 | N | LYS | E | 407 | 83.551 | −33.679 | −17.622 | 1.00 | 52.82 | E |
| ATOM | 17700 | CA | LYS | E | 407 | 83.437 | −35.128 | −17.749 | 1.00 | 48.42 | E |
| ATOM | 17701 | CB | LYS | E | 407 | 84.443 | −35.855 | −16.863 | 1.00 | 100.03 | E |
| ATOM | 17702 | CG | LYS | E | 407 | 85.886 | −35.601 | −17.222 | 1.00 | 86.13 | E |
| ATOM | 17703 | CD | LYS | E | 407 | 86.799 | −36.512 | −16.421 | 1.00 | 86.13 | E |
| ATOM | 17704 | CE | LYS | E | 407 | 88.274 | −36.169 | −16.632 | 1.00 | 86.13 | E |
| ATOM | 17705 | NZ | LYS | E | 407 | 88.630 | −34.835 | −16.066 | 1.00 | 86.13 | E |
| ATOM | 17706 | C | LYS | E | 407 | 82.041 | −35.458 | −17.285 | 1.00 | 57.73 | E |
| ATOM | 17707 | O | LYS | E | 407 | 81.553 | −34.836 | −16.344 | 1.00 | 56.67 | E |
| ATOM | 17708 | N | LYS | E | 408 | 81.378 | −36.397 | −17.959 | 1.00 | 61.97 | E |
| ATOM | 17709 | CA | LYS | E | 408 | 80.038 | −36.779 | −17.541 | 1.00 | 61.56 | E |
| ATOM | 17710 | CB | LYS | E | 408 | 79.341 | −37.589 | −18.625 | 1.00 | 61.09 | E |
| ATOM | 17711 | CG | LYS | E | 408 | 77.900 | −37.899 | −18.295 | 1.00 | 59.89 | E |
| ATOM | 17712 | CD | LYS | E | 408 | 77.185 | −38.443 | −19.505 | 1.00 | 59.89 | E |
| ATOM | 17713 | CE | LYS | E | 408 | 75.713 | −38.663 | −19.226 | 1.00 | 59.89 | E |
| ATOM | 17714 | NZ | LYS | E | 408 | 74.979 | −39.085 | −20.453 | 1.00 | 59.89 | E |
| ATOM | 17715 | C | LYS | E | 408 | 80.273 | −37.629 | −16.310 | 1.00 | 61.69 | E |
| ATOM | 17716 | O | LYS | E | 408 | 80.787 | −38.731 | −16.405 | 1.00 | 60.26 | E |
| ATOM | 17717 | N | ASP | E | 409 | 79.929 | −37.119 | −15.142 | 1.00 | 67.28 | E |
| ATOM | 17718 | CA | ASP | E | 409 | 80.183 | −37.893 | −13.946 | 1.00 | 67.46 | E |
| ATOM | 17719 | CB | ASP | E | 409 | 79.944 | −37.050 | −12.675 | 1.00 | 88.94 | E |
| ATOM | 17720 | CG | ASP | E | 409 | 78.643 | −36.270 | −12.706 | 1.00 | 81.76 | E |
| ATOM | 17721 | OD1 | ASP | E | 409 | 77.602 | −36.860 | −13.059 | 1.00 | 81.76 | E |
| ATOM | 17722 | OD2 | ASP | E | 409 | 78.665 | −35.070 | −12.358 | 1.00 | 81.76 | E |
| ATOM | 17723 | C | ASP | E | 409 | 79.417 | −39.212 | −13.891 | 1.00 | 67.37 | E |
| ATOM | 17724 | O | ASP | E | 409 | 78.466 | −39.420 | −14.648 | 1.00 | 71.10 | E |
| ATOM | 17725 | N | LYS | E | 410 | 79.883 | −40.094 | −13.001 | 1.00 | 124.91 | E |
| ATOM | 17726 | CA | LYS | E | 410 | 79.338 | −41.433 | −12.757 | 1.00 | 131.35 | E |
| ATOM | 17727 | CB | LYS | E | 410 | 79.487 | −41.796 | −11.276 | 1.00 | 180.14 | E |
| ATOM | 17728 | CG | LYS | E | 410 | 78.612 | −40.977 | −10.322 | 1.00 | 119.59 | E |
| ATOM | 17729 | CD | LYS | E | 410 | 79.091 | −39.532 | −10.174 | 1.00 | 119.59 | E |
| ATOM | 17730 | CE | LYS | E | 410 | 78.166 | −38.728 | −9.260 | 1.00 | 119.59 | E |
| ATOM | 17731 | NZ | LYS | E | 410 | 78.610 | −37.310 | −9.092 | 1.00 | 119.59 | E |
| ATOM | 17732 | C | LYS | E | 410 | 77.887 | −41.629 | −13.161 | 1.00 | 130.17 | E |
| ATOM | 17733 | O | LYS | E | 410 | 77.111 | −40.680 | −13.198 | 1.00 | 129.70 | E |
| ATOM | 17734 | N | ASP | E | 411 | 77.527 | −42.879 | −13.432 | 1.00 | 78.35 | E |
| ATOM | 17735 | CA | ASP | E | 411 | 76.171 | −43.240 | −13.852 | 1.00 | 75.86 | E |
| ATOM | 17736 | CB | ASP | E | 411 | 75.774 | −44.598 | −13.269 | 1.00 | 145.28 | E |
| ATOM | 17737 | CG | ASP | E | 411 | 76.184 | −45.754 | −14.159 | 1.00 | 145.28 | E |
| ATOM | 17738 | OD1 | ASP | E | 411 | 75.856 | −45.725 | −15.366 | 1.00 | 145.28 | E |
| ATOM | 17739 | OD2 | ASP | E | 411 | 76.828 | −46.695 | −13.655 | 1.00 | 145.28 | E |
| ATOM | 17740 | C | ASP | E | 411 | 75.085 | −42.219 | −13.527 | 1.00 | 77.47 | E |
| ATOM | 17741 | O | ASP | E | 411 | 74.385 | −42.336 | −12.521 | 1.00 | 81.65 | E |
| ATOM | 17742 | N | ALA | E | 412 | 74.944 | −41.229 | −14.406 | 1.00 | 82.41 | E |
| ATOM | 17743 | CA | ALA | E | 412 | 73.958 | −40.172 | −14.240 | 1.00 | 95.43 | E |
| ATOM | 17744 | CB | ALA | E | 412 | 74.323 | −39.309 | −13.041 | 1.00 | 51.80 | E |
| ATOM | 17745 | C | ALA | E | 412 | 73.906 | −39.323 | −15.505 | 1.00 | 90.13 | E |
| ATOM | 17746 | O | ALA | E | 412 | 74.717 | −39.500 | −16.416 | 1.00 | 85.18 | E |
| ATOM | 17747 | N | ASN | E | 413 | 72.950 | −38.401 | −15.554 | 1.00 | 72.38 | E |
| ATOM | 17748 | CA | ASN | E | 413 | 72.792 | −37.528 | −16.711 | 1.00 | 65.19 | E |
| ATOM | 17749 | CB | ASN | E | 413 | 71.321 | −37.177 | −16.901 | 1.00 | 64.39 | E |
| ATOM | 17750 | CG | ASN | E | 413 | 70.470 | −38.388 | −17.168 | 1.00 | 64.39 | E |
| ATOM | 17751 | OD1 | ASN | E | 413 | 70.850 | −39.264 | −17.953 | 1.00 | 64.39 | E |
| ATOM | 17752 | ND2 | ASN | E | 413 | 69.302 | −38.443 | −16.532 | 1.00 | 64.39 | E |
| ATOM | 17753 | C | ASN | E | 413 | 73.571 | −36.243 | −16.552 | 1.00 | 69.77 | E |
| ATOM | 17754 | O | ASN | E | 413 | 73.768 | −35.500 | −17.501 | 1.00 | 69.46 | E |
| ATOM | 17755 | N | THR | E | 414 | 74.024 | −36.003 | −15.337 | 1.00 | 45.86 | E |
| ATOM | 17756 | CA | THR | E | 414 | 74.731 | −34.789 | −14.999 | 1.00 | 48.90 | E |
| ATOM | 17757 | CB | THR | E | 414 | 74.543 | −34.524 | −13.506 | 1.00 | 86.43 | E |
| ATOM | 17758 | OG1 | THR | E | 414 | 75.238 | −33.339 | −13.150 | 1.00 | 56.92 | E |
| ATOM | 17759 | CG2 | THR | E | 414 | 75.056 | −35.681 | −12.689 | 1.00 | 56.92 | E |
| ATOM | 17760 | C | THR | E | 414 | 76.213 | −34.768 | −15.387 | 1.00 | 48.36 | E |
| ATOM | 17761 | O | THR | E | 414 | 76.774 | −35.797 | −15.731 | 1.00 | 49.43 | E |
| ATOM | 17762 | N | TYR | E | 415 | 76.826 | −33.582 | −15.354 | 1.00 | 48.07 | E |
| ATOM | 17763 | CA | TYR | E | 415 | 78.237 | −33.397 | −15.719 | 1.00 | 49.10 | E |
| ATOM | 17764 | CB | TYR | E | 415 | 78.376 | −32.625 | −17.040 | 1.00 | 66.06 | E |
| ATOM | 17765 | CG | TYR | E | 415 | 77.859 | −33.341 | −18.261 | 1.00 | 33.00 | E |
| ATOM | 17766 | CD1 | TYR | E | 415 | 76.494 | −33.553 | −18.447 | 1.00 | 33.00 | E |
| ATOM | 17767 | CE1 | TYR | E | 415 | 76.018 | −34.229 | −19.561 | 1.00 | 33.00 | E |
| ATOM | 17768 | CD2 | TYR | E | 415 | 78.740 | −33.826 | −19.229 | 1.00 | 33.00 | E |
| ATOM | 17769 | CE2 | TYR | E | 415 | 78.268 | −34.510 | −20.355 | 1.00 | 33.00 | E |
| ATOM | 17770 | CZ | TYR | E | 415 | 76.909 | −34.706 | −20.509 | 1.00 | 33.00 | E |
| ATOM | 17771 | OH | TYR | E | 415 | 76.445 | −35.390 | −21.605 | 1.00 | 33.00 | E |
| ATOM | 17772 | C | TYR | E | 415 | 78.983 | −32.607 | −14.657 | 1.00 | 56.66 | E |
| ATOM | 17773 | O | TYR | E | 415 | 78.370 | −31.927 | −13.838 | 1.00 | 51.31 | E |
| ATOM | 17774 | N | SER | E | 416 | 80.312 | −32.678 | −14.703 | 1.00 | 38.80 | E |
| ATOM | 17775 | CA | SER | E | 416 | 81.162 | −31.971 | −13.747 | 1.00 | 36.75 | E |
| ATOM | 17776 | CB | SER | E | 416 | 81.704 | −32.924 | −12.695 | 1.00 | 65.39 | E |
| ATOM | 17777 | OG | SER | E | 416 | 80.681 | −33.274 | −11.791 | 1.00 | 41.77 | E |
| ATOM | 17778 | C | SER | E | 416 | 82.313 | −31.296 | −14.458 | 1.00 | 42.94 | E |
| ATOM | 17779 | O | SER | E | 416 | 82.787 | −31.774 | −15.495 | 1.00 | 41.31 | E |
| ATOM | 17780 | N | LEU | E | 417 | 82.761 | −30.184 | −13.884 | 1.00 | 47.44 | E |
| ATOM | 17781 | CA | LEU | E | 417 | 83.832 | −29.383 | −14.463 | 1.00 | 46.34 | E |
| ATOM | 17782 | CB | LEU | E | 417 | 83.222 | −28.146 | −15.125 | 1.00 | 98.23 | E |
| ATOM | 17783 | CG | LEU | E | 417 | 84.130 | −26.920 | −15.223 | 1.00 | 56.34 | E |
| ATOM | 17784 | CD1 | LEU | E | 417 | 85.099 | −27.100 | −16.371 | 1.00 | 56.34 | E |
| ATOM | 17785 | CD2 | LEU | E | 417 | 83.300 | −25.681 | −15.426 | 1.00 | 56.34 | E |
| ATOM | 17786 | C | LEU | E | 417 | 84.886 | −28.928 | −13.455 | 1.00 | 57.85 | E |
| ATOM | 17787 | O | LEU | E | 417 | 84.602 | −28.725 | −12.269 | 1.00 | 52.95 | E |
| ATOM | 17788 | N | THR | E | 418 | 86.102 | −28.747 | −13.951 | 1.00 | 42.48 | E |
| ATOM | 17789 | CA | THR | E | 418 | 87.211 | −28.290 | −13.130 | 1.00 | 35.54 | E |
| ATOM | 17790 | CB | THR | E | 418 | 88.145 | −29.452 | −12.765 | 1.00 | 74.00 | E |
| ATOM | 17791 | OG1 | THR | E | 418 | 87.493 | −30.311 | −11.824 | 1.00 | 42.41 | E |
| ATOM | 17792 | CG2 | THR | E | 418 | 89.437 | −28.928 | −12.164 | 1.00 | 42.41 | E |
| ATOM | 17793 | C | THR | E | 418 | 88.006 | −27.261 | −13.917 | 1.00 | 50.73 | E |
| ATOM | 17794 | O | THR | E | 418 | 88.588 | −27.590 | −14.942 | 1.00 | 44.20 | E |
| ATOM | 17795 | N | ASP | E | 419 | 88.031 | −26.017 | −13.458 | 1.00 | 47.32 | E |
| ATOM | 17796 | CA | ASP | E | 419 | 88.778 | −24.987 | −14.173 | 1.00 | 52.43 | E |
| ATOM | 17797 | CB | ASP | E | 419 | 87.848 | −23.857 | −14.605 | 1.00 | 95.00 | E |
| ATOM | 17798 | CG | ASP | E | 419 | 88.490 | −22.950 | −15.619 | 1.00 | 72.59 | E |
| ATOM | 17799 | OD1 | ASP | E | 419 | 89.072 | −23.469 | −16.591 | 1.00 | 72.59 | E |
| ATOM | 17800 | OD2 | ASP | E | 419 | 88.417 | −21.721 | −15.458 | 1.00 | 72.59 | E |
| ATOM | 17801 | C | ASP | E | 419 | 89.898 | −24.448 | −13.298 | 1.00 | 56.52 | E |
| ATOM | 17802 | O | ASP | E | 419 | 89.658 | −23.988 | −12.178 | 1.00 | 51.41 | E |
| ATOM | 17803 | N | LEU | E | 420 | 91.125 | −24.507 | −13.809 | 1.00 | 57.17 | E |
| ATOM | 17804 | CA | LEU | E | 420 | 92.298 | −24.052 | −13.065 | 1.00 | 61.86 | E |
| ATOM | 17805 | CB | LEU | E | 420 | 93.396 | −25.106 | −13.174 | 1.00 | 115.36 | E |
| ATOM | 17806 | CG | LEU | E | 420 | 94.494 | −25.083 | −12.118 | 1.00 | 74.31 | E |
| ATOM | 17807 | CD1 | LEU | E | 420 | 93.888 | −25.412 | −10.771 | 1.00 | 74.31 | E |
| ATOM | 17808 | CD2 | LEU | E | 420 | 95.577 | −26.093 | −12.482 | 1.00 | 74.31 | E |
| ATOM | 17809 | C | LEU | E | 420 | 92.824 | −22.703 | −13.568 | 1.00 | 64.43 | E |
| ATOM | 17810 | O | LEU | E | 420 | 92.633 | −22.347 | −14.730 | 1.00 | 63.75 | E |
| ATOM | 17811 | N | VAL | E | 421 | 93.485 | −21.951 | −12.691 | 1.00 | 68.91 | E |
| ATOM | 17812 | CA | VAL | E | 421 | 94.034 | −20.646 | −13.063 | 1.00 | 67.42 | E |
| ATOM | 17813 | CB | VAL | E | 421 | 93.057 | −19.511 | −12.706 | 1.00 | 101.25 | E |
| ATOM | 17814 | CG1 | VAL | E | 421 | 93.692 | −18.163 | −12.969 | 1.00 | 56.68 | E |
| ATOM | 17815 | CG2 | VAL | E | 421 | 91.817 | −19.644 | −13.534 | 1.00 | 56.68 | E |
| ATOM | 17816 | C | VAL | E | 421 | 95.379 | −20.358 | −12.399 | 1.00 | 69.68 | E |
| ATOM | 17817 | O | VAL | E | 421 | 95.552 | −20.593 | −11.211 | 1.00 | 65.28 | E |
| ATOM | 17818 | N | ARG | E | 422 | 96.322 | −19.840 | −13.184 | 1.00 | 85.55 | E |
| ATOM | 17819 | CA | ARG | E | 422 | 97.664 | −19.505 | −12.705 | 1.00 | 87.68 | E |
| ATOM | 17820 | CB | ARG | E | 422 | 98.633 | −19.484 | −13.886 | 1.00 | 149.51 | E |
| ATOM | 17821 | CG | ARG | E | 422 | 99.844 | −20.380 | −13.730 | 1.00 | 110.10 | E |
| ATOM | 17822 | CD | ARG | E | 422 | 100.894 | −19.772 | −12.824 | 1.00 | 110.10 | E |
| ATOM | 17823 | NE | ARG | E | 422 | 102.110 | −20.582 | −12.813 | 1.00 | 110.10 | E |
| ATOM | 17824 | CZ | ARG | E | 422 | 103.252 | −20.213 | −12.241 | 1.00 | 110.10 | E |
| ATOM | 17825 | NH1 | ARG | E | 422 | 103.341 | −19.041 | −11.626 | 1.00 | 110.10 | E |
| ATOM | 17826 | NH2 | ARG | E | 422 | 104.308 | −21.013 | −12.290 | 1.00 | 110.10 | E |
| ATOM | 17827 | C | ARG | E | 422 | 97.644 | −18.134 | −12.047 | 1.00 | 84.91 | E |
| ATOM | 17828 | O | ARG | E | 422 | 97.664 | −17.118 | −12.736 | 1.00 | 75.58 | E |
| ATOM | 17829 | N | THR | E | 423 | 97.606 | −18.106 | −10.718 | 1.00 | 139.09 | E |
| ATOM | 17830 | CA | THR | E | 423 | 97.568 | −16.842 | −9.978 | 1.00 | 148.74 | E |
| ATOM | 17831 | CB | THR | E | 423 | 97.225 | −17.067 | −8.477 | 1.00 | 200.84 | E |
| ATOM | 17832 | OG1 | THR | E | 423 | 95.987 | −17.777 | −8.366 | 1.00 | 162.91 | E |
| ATOM | 17833 | CG2 | THR | E | 423 | 97.091 | −15.731 | −7.747 | 1.00 | 162.91 | E |
| ATOM | 17834 | C | THR | E | 423 | 98.889 | −16.086 | −10.050 | 1.00 | 146.96 | E |
| ATOM | 17835 | O | THR | E | 423 | 99.538 | −15.881 | −9.028 | 1.00 | 145.45 | E |
| ATOM | 17836 | N | THR | E | 424 | 99.289 | −15.674 | −11.249 | 1.00 | 180.69 | E |
| ATOM | 17837 | CA | THR | E | 424 | 100.535 | −14.932 | −11.402 | 1.00 | 180.69 | E |
| ATOM | 17838 | CB | THR | E | 424 | 100.839 | −14.642 | −12.880 | 1.00 | 170.66 | E |
| ATOM | 17839 | OG1 | THR | E | 424 | 100.889 | −15.879 | −13.598 | 1.00 | 116.47 | E |
| ATOM | 17840 | CG2 | THR | E | 424 | 102.179 | −13.932 | −13.023 | 1.00 | 116.47 | E |
| ATOM | 17841 | C | THR | E | 424 | 100.416 | −13.614 | −10.644 | 1.00 | 180.69 | E |
| ATOM | 17842 | O | THR | E | 424 | 99.313 | −13.114 | −10.426 | 1.00 | 180.69 | E |
| ATOM | 17843 | N | SER | E | 425 | 101.552 | −13.053 | −10.244 | 1.00 | 148.96 | E |
| ATOM | 17844 | CA | SER | E | 425 | 101.565 | −11.807 | −9.487 | 1.00 | 148.96 | E |
| ATOM | 17845 | CB | SER | E | 425 | 102.938 | −11.609 | −8.843 | 1.00 | 200.84 | E |
| ATOM | 17846 | OG | SER | E | 425 | 102.943 | −10.463 | −8.013 | 1.00 | 102.27 | E |
| ATOM | 17847 | C | SER | E | 425 | 101.209 | −10.577 | −10.314 | 1.00 | 148.96 | E |
| ATOM | 17848 | O | SER | E | 425 | 101.159 | −9.468 | −9.788 | 1.00 | 148.96 | E |
| ATOM | 17849 | N | LYS | E | 426 | 100.960 | −10.774 | −11.604 | 1.00 | 178.72 | E |
| ATOM | 17850 | CA | LYS | E | 426 | 100.612 | −9.668 | −12.491 | 1.00 | 178.72 | E |
| ATOM | 17851 | CB | LYS | E | 426 | 100.333 | −10.190 | −13.901 | 1.00 | 181.31 | E |
| ATOM | 17852 | CG | LYS | E | 426 | 101.422 | −11.073 | −14.469 | 1.00 | 125.61 | E |
| ATOM | 17853 | CD | LYS | E | 426 | 100.975 | −11.690 | −15.781 | 1.00 | 125.61 | E |
| ATOM | 17854 | CE | LYS | E | 426 | 101.989 | −12.692 | −16.294 | 1.00 | 125.61 | E |
| ATOM | 17855 | NZ | LYS | E | 426 | 101.527 | −13.316 | −17.559 | 1.00 | 125.61 | E |
| ATOM | 17856 | C | LYS | E | 426 | 99.378 | −8.926 | −11.987 | 1.00 | 178.72 | E |
| ATOM | 17857 | O | LYS | E | 426 | 99.305 | −7.699 | −12.059 | 1.00 | 178.72 | E |
| ATOM | 17858 | N | MET | E | 427 | 98.411 | −9.685 | −11.479 | 1.00 | 157.39 | E |
| ATOM | 17859 | CA | MET | E | 427 | 97.163 | −9.124 | −10.973 | 1.00 | 157.39 | E |
| ATOM | 17860 | CB | MET | E | 427 | 96.177 | −10.250 | −10.667 | 1.00 | 143.86 | E |
| ATOM | 17861 | CG | MET | E | 427 | 96.741 | −11.329 | −9.768 | 1.00 | 143.86 | E |
| ATOM | 17862 | SD | MET | E | 427 | 95.545 | −12.634 | −9.485 | 1.00 | 143.86 | E |
| ATOM | 17863 | CE | MET | E | 427 | 95.674 | −13.557 | −11.031 | 1.00 | 143.86 | E |
| ATOM | 17864 | C | MET | E | 427 | 97.346 | −8.258 | −9.735 | 1.00 | 157.39 | E |
| ATOM | 17865 | O | MET | E | 427 | 98.452 | −7.808 | −9.437 | 1.00 | 157.39 | E |
| ATOM | 17866 | N | SER | E | 428 | 96.251 | −8.029 | −9.016 | 1.00 | 127.74 | E |
| ATOM | 17867 | CA | SER | E | 428 | 96.288 | −7.206 | −7.815 | 1.00 | 129.39 | E |
| ATOM | 17868 | CB | SER | E | 428 | 96.570 | −5.750 | −8.194 | 1.00 | 103.06 | E |
| ATOM | 17869 | OG | SER | E | 428 | 95.531 | −5.226 | −9.006 | 1.00 | 99.77 | E |
| ATOM | 17870 | C | SER | E | 428 | 94.992 | −7.271 | −7.007 | 1.00 | 125.34 | E |
| ATOM | 17871 | O | SER | E | 428 | 94.622 | −6.300 | −6.349 | 1.00 | 125.22 | E |
| ATOM | 17872 | N | SER | E | 429 | 94.303 | −8.408 | −7.050 | 1.00 | 134.96 | E |
| ATOM | 17873 | CA | SER | E | 429 | 93.055 | −8.558 | −6.305 | 1.00 | 133.63 | E |
| ATOM | 17874 | CB | SER | E | 429 | 91.911 | −7.844 | −7.030 | 1.00 | 92.18 | E |
| ATOM | 17875 | OG | SER | E | 429 | 92.132 | −6.451 | −7.108 | 1.00 | 92.18 | E |
| ATOM | 17876 | C | SER | E | 429 | 92.658 | −10.013 | −6.090 | 1.00 | 132.01 | E |
| ATOM | 17877 | O | SER | E | 429 | 91.729 | −10.305 | −5.338 | 1.00 | 120.71 | E |
| ATOM | 17878 | N | GLN | E | 430 | 93.366 | −10.925 | −6.742 | 1.00 | 78.17 | E |
| ATOM | 17879 | CA | GLN | E | 430 | 93.043 | −12.345 | −6.636 | 1.00 | 78.17 | E |
| ATOM | 17880 | CB | GLN | E | 430 | 93.019 | −12.812 | −5.185 | 1.00 | 156.27 | E |
| ATOM | 17881 | CG | GLN | E | 430 | 92.755 | −14.291 | −5.080 | 1.00 | 85.26 | E |
| ATOM | 17882 | CD | GLN | E | 430 | 92.682 | −14.769 | −3.659 | 1.00 | 85.26 | E |
| ATOM | 17883 | OE1 | GLN | E | 430 | 91.743 | −14.446 | −2.932 | 1.00 | 85.26 | E |
| ATOM | 17884 | NE2 | GLN | E | 430 | 93.681 | −15.544 | −3.244 | 1.00 | 85.26 | E |
| ATOM | 17885 | C | GLN | E | 430 | 91.669 | −12.560 | −7.265 | 1.00 | 78.17 | E |
| ATOM | 17886 | O | GLN | E | 430 | 90.645 | −12.127 | −6.736 | 1.00 | 78.17 | E |
| ATOM | 17887 | N | PRO | E | 431 | 91.632 | −13.260 | −8.401 | 1.00 | 53.33 | E |
| ATOM | 17888 | CD | PRO | E | 431 | 92.687 | −14.113 | −8.979 | 1.00 | 51.75 | E |
| ATOM | 17889 | CA | PRO | E | 431 | 90.364 | −13.495 | −9.068 | 1.00 | 53.32 | E |
| ATOM | 17890 | CB | PRO | E | 431 | 90.776 | −14.281 | −10.305 | 1.00 | 150.71 | E |
| ATOM | 17891 | CG | PRO | E | 431 | 91.893 | −15.107 | −9.792 | 1.00 | 121.27 | E |
| ATOM | 17892 | C | PRO | E | 431 | 89.361 | −14.237 | −8.231 | 1.00 | 49.94 | E |
| ATOM | 17893 | O | PRO | E | 431 | 89.703 | −14.928 | −7.277 | 1.00 | 49.82 | E |
| ATOM | 17894 | N | VAL | E | 432 | 88.108 | −14.057 | −8.613 | 1.00 | 47.92 | E |
| ATOM | 17895 | CA | VAL | E | 432 | 86.984 | −14.714 | −7.994 | 1.00 | 50.30 | E |
| ATOM | 17896 | CB | VAL | E | 432 | 85.996 | −13.682 | −7.453 | 1.00 | 84.96 | E |
| ATOM | 17897 | CG1 | VAL | E | 432 | 85.477 | −12.820 | −8.570 | 1.00 | 57.04 | E |
| ATOM | 17898 | CG2 | VAL | E | 432 | 84.870 | −14.371 | −6.777 | 1.00 | 57.04 | E |
| ATOM | 17899 | C | VAL | E | 432 | 86.382 | −15.498 | −9.171 | 1.00 | 48.30 | E |
| ATOM | 17900 | O | VAL | E | 432 | 86.507 | −15.086 | −10.323 | 1.00 | 38.66 | E |
| ATOM | 17901 | N | CYS | E | 433 | 85.748 | −16.630 | −8.909 | 1.00 | 59.05 | E |
| ATOM | 17902 | CA | CYS | E | 433 | 85.196 | −17.415 | −10.004 | 1.00 | 54.93 | E |
| ATOM | 17903 | C | CYS | E | 433 | 83.732 | −17.102 | −10.258 | 1.00 | 52.57 | E |
| ATOM | 17904 | O | CYS | E | 433 | 82.908 | −17.193 | −9.348 | 1.00 | 53.31 | E |
| ATOM | 17905 | CB | CYS | E | 433 | 85.369 | −18.912 | −9.710 | 1.00 | 146.01 | E |
| ATOM | 17906 | SG | CYS | E | 433 | 84.854 | −20.030 | −11.057 | 1.00 | 93.12 | E |
| ATOM | 17907 | N | VAL | E | 434 | 83.415 | −16.707 | −11.490 | 1.00 | 48.05 | E |
| ATOM | 17908 | CA | VAL | E | 434 | 82.029 | −16.432 | −11.873 | 1.00 | 44.04 | E |
| ATOM | 17909 | CB | VAL | E | 434 | 81.890 | −15.113 | −12.650 | 1.00 | 57.28 | E |
| ATOM | 17910 | CG1 | VAL | E | 434 | 80.424 | −14.793 | −12.839 | 1.00 | 39.06 | E |
| ATOM | 17911 | CG2 | VAL | E | 434 | 82.564 | −13.990 | −11.908 | 1.00 | 39.06 | E |
| ATOM | 17912 | C | VAL | E | 434 | 81.568 | −17.580 | −12.775 | 1.00 | 54.44 | E |
| ATOM | 17913 | O | VAL | E | 434 | 82.129 | −17.807 | −13.849 | 1.00 | 45.50 | E |
| ATOM | 17914 | N | VAL | E | 435 | 80.545 | −18.301 | −12.330 | 1.00 | 45.34 | E |
| ATOM | 17915 | CA | VAL | E | 435 | 80.026 | −19.448 | −13.073 | 1.00 | 37.08 | E |
| ATOM | 17916 | CB | VAL | E | 435 | 79.789 | −20.637 | −12.109 | 1.00 | 58.76 | E |
| ATOM | 17917 | CG1 | VAL | E | 435 | 79.033 | −21.759 | −12.799 | 1.00 | 38.09 | E |
| ATOM | 17918 | CG2 | VAL | E | 435 | 81.112 | −21.135 | −11.594 | 1.00 | 38.09 | E |
| ATOM | 17919 | C | VAL | E | 435 | 78.725 | −19.164 | −13.824 | 1.00 | 45.00 | E |
| ATOM | 17920 | O | VAL | E | 435 | 77.730 | −18.767 | −13.223 | 1.00 | 45.59 | E |
| ATOM | 17921 | N | PHE | E | 436 | 78.740 | −19.371 | −15.140 | 1.00 | 36.06 | E |
| ATOM | 17922 | CA | PHE | E | 436 | 77.549 | −19.156 | −15.954 | 1.00 | 32.26 | E |
| ATOM | 17923 | CB | PHE | E | 436 | 77.893 | −18.375 | −17.217 | 1.00 | 70.78 | E |
| ATOM | 17924 | CG | PHE | E | 436 | 78.395 | −16.993 | −16.951 | 1.00 | 45.55 | E |
| ATOM | 17925 | CD1 | PHE | E | 436 | 79.747 | −16.760 | −16.713 | 1.00 | 45.55 | E |
| ATOM | 17926 | CD2 | PHE | E | 436 | 77.512 | −15.912 | −16.929 | 1.00 | 45.55 | E |
| ATOM | 17927 | CE1 | PHE | E | 436 | 80.218 | −15.462 | −16.459 | 1.00 | 45.55 | E |
| ATOM | 17928 | CE2 | PHE | E | 436 | 77.970 | −14.607 | −16.671 | 1.00 | 45.55 | E |
| ATOM | 17929 | CZ | PHE | E | 436 | 79.323 | −14.385 | −16.439 | 1.00 | 45.55 | E |
| ATOM | 17930 | C | PHE | E | 436 | 76.928 | −20.491 | −16.333 | 1.00 | 40.09 | E |
| ATOM | 17931 | O | PHE | E | 436 | 77.565 | −21.324 | −16.964 | 1.00 | 39.47 | E |
| ATOM | 17932 | N | HIS | E | 437 | 75.678 | −20.687 | −15.934 | 1.00 | 37.47 | E |
| ATOM | 17933 | CA | HIS | E | 437 | 74.957 | −21.922 | −16.222 | 1.00 | 38.54 | E |
| ATOM | 17934 | CB | HIS | E | 437 | 75.277 | −22.961 | −15.158 | 1.00 | 67.68 | E |
| ATOM | 17935 | CG | HIS | E | 437 | 74.594 | −24.265 | −15.381 | 1.00 | 44.05 | E |
| ATOM | 17936 | CD2 | HIS | E | 437 | 73.459 | −24.781 | −14.854 | 1.00 | 44.05 | E |
| ATOM | 17937 | ND1 | HIS | E | 437 | 75.054 | −25.196 | −16.285 | 1.00 | 44.05 | E |
| ATOM | 17938 | CE1 | HIS | E | 437 | 74.234 | −26.232 | −16.307 | 1.00 | 44.05 | E |
| ATOM | 17939 | NE2 | HIS | E | 437 | 73.257 | −26.005 | −15.448 | 1.00 | 44.05 | E |
| ATOM | 17940 | C | HIS | E | 437 | 73.454 | −21.639 | −16.235 | 1.00 | 42.85 | E |
| ATOM | 17941 | O | HIS | E | 437 | 73.003 | −20.647 | −15.676 | 1.00 | 45.07 | E |
| ATOM | 17942 | N | ASP | E | 438 | 72.677 | −22.504 | −16.873 | 1.00 | 43.07 | E |
| ATOM | 17943 | CA | ASP | E | 438 | 71.231 | −22.308 | −16.946 | 1.00 | 44.98 | E |
| ATOM | 17944 | CB | ASP | E | 438 | 70.577 | −23.476 | −17.687 | 1.00 | 85.18 | E |
| ATOM | 17945 | CG | ASP | E | 438 | 70.606 | −23.288 | −19.194 | 1.00 | 56.14 | E |
| ATOM | 17946 | OD1 | ASP | E | 438 | 71.459 | −22.503 | −19.675 | 1.00 | 56.14 | E |
| ATOM | 17947 | OD2 | ASP | E | 438 | 69.784 | −23.924 | −19.896 | 1.00 | 56.14 | E |
| ATOM | 17948 | C | ASP | E | 438 | 70.617 | −22.120 | −15.567 | 1.00 | 46.32 | E |
| ATOM | 17949 | O | ASP | E | 438 | 69.820 | −21.208 | −15.365 | 1.00 | 49.88 | E |
| ATOM | 17950 | N | THR | E | 439 | 70.967 | −22.990 | −14.625 | 1.00 | 78.46 | E |
| ATOM | 17951 | CA | THR | E | 439 | 70.481 | −22.849 | −13.259 | 1.00 | 75.81 | E |
| ATOM | 17952 | CB | THR | E | 439 | 70.673 | −24.150 | −12.485 | 1.00 | 67.54 | E |
| ATOM | 17953 | OG1 | THR | E | 439 | 72.069 | −24.453 | −12.410 | 1.00 | 53.35 | E |
| ATOM | 17954 | CG2 | THR | E | 439 | 69.975 | −25.296 | −13.204 | 1.00 | 53.35 | E |
| ATOM | 17955 | C | THR | E | 439 | 71.438 | −21.774 | −12.755 | 1.00 | 81.58 | E |
| ATOM | 17956 | O | THR | E | 439 | 72.589 | −21.752 | −13.166 | 1.00 | 81.92 | E |
| ATOM | 17957 | N | LEU | E | 440 | 70.987 | −20.881 | −11.887 | 1.00 | 75.68 | E |
| ATOM | 17958 | CA | LEU | E | 440 | 71.845 | −19.786 | −11.414 | 1.00 | 77.70 | E |
| ATOM | 17959 | CB | LEU | E | 440 | 72.984 | −20.287 | −10.477 | 1.00 | 78.85 | E |
| ATOM | 17960 | CG | LEU | E | 440 | 74.205 | −21.241 | −10.611 | 1.00 | 58.85 | E |
| ATOM | 17961 | CD1 | LEU | E | 440 | 73.713 | −22.644 | −10.756 | 1.00 | 58.85 | E |
| ATOM | 17962 | CD2 | LEU | E | 440 | 75.141 | −20.886 | −11.744 | 1.00 | 58.85 | E |
| ATOM | 17963 | C | LEU | E | 440 | 72.445 | −18.979 | −12.570 | 1.00 | 87.65 | E |
| ATOM | 17964 | O | LEU | E | 440 | 73.622 | −19.132 | −12.871 | 1.00 | 85.87 | E |
| ATOM | 17965 | N | GLU | E | 441 | 71.639 | −18.115 | −13.202 | 1.00 | 48.75 | E |
| ATOM | 17966 | CA | GLU | E | 441 | 72.087 | −17.295 | −14.347 | 1.00 | 48.75 | E |
| ATOM | 17967 | CB | GLU | E | 441 | 71.240 | −16.026 | −14.446 | 1.00 | 200.84 | E |
| ATOM | 17968 | CG | GLU | E | 441 | 69.777 | −16.295 | −14.764 | 1.00 | 136.56 | E |
| ATOM | 17969 | CD | GLU | E | 441 | 68.993 | −15.019 | −15.016 | 1.00 | 136.56 | E |
| ATOM | 17970 | OE1 | GLU | E | 441 | 68.966 | −14.147 | −14.120 | 1.00 | 136.56 | E |
| ATOM | 17971 | OE2 | GLU | E | 441 | 68.404 | −14.888 | −16.112 | 1.00 | 136.56 | E |
| ATOM | 17972 | C | GLU | E | 441 | 73.583 | −16.923 | −14.336 | 1.00 | 48.75 | E |
| ATOM | 17973 | O | GLU | E | 441 | 74.263 | −17.016 | −15.361 | 1.00 | 48.75 | E |
| ATOM | 17974 | N | ALA | E | 442 | 74.090 | −16.513 | −13.176 | 1.00 | 55.67 | E |
| ATOM | 17975 | CA | ALA | E | 442 | 75.496 | −16.159 | −13.040 | 1.00 | 53.89 | E |
| ATOM | 17976 | CB | ALA | E | 442 | 75.802 | −14.909 | −13.843 | 1.00 | 30.49 | E |
| ATOM | 17977 | C | ALA | E | 442 | 75.830 | −15.921 | −11.584 | 1.00 | 49.88 | E |
| ATOM | 17978 | O | ALA | E | 442 | 75.433 | −14.905 | −11.024 | 1.00 | 47.29 | E |
| ATOM | 17979 | N | GLN | E | 443 | 76.553 | −16.851 | −10.964 | 1.00 | 59.28 | E |
| ATOM | 17980 | CA | GLN | E | 443 | 76.933 | −16.702 | −9.558 | 1.00 | 62.56 | E |
| ATOM | 17981 | CB | GLN | E | 443 | 76.473 | −17.903 | −8.738 | 1.00 | 78.66 | E |
| ATOM | 17982 | CG | GLN | E | 443 | 75.097 | −17.739 | −8.141 | 1.00 | 61.89 | E |
| ATOM | 17983 | CD | GLN | E | 443 | 74.869 | −18.680 | −6.977 | 1.00 | 61.89 | E |
| ATOM | 17984 | OE1 | GLN | E | 443 | 75.682 | −18.744 | −6.045 | 1.00 | 61.89 | E |
| ATOM | 17985 | NE2 | GLN | E | 443 | 73.757 | −19.414 | −7.016 | 1.00 | 61.89 | E |
| ATOM | 17986 | C | GLN | E | 443 | 78.428 | −16.512 | −9.365 | 1.00 | 64.73 | E |
| ATOM | 17987 | O | GLN | E | 443 | 79.238 | −17.003 | −10.155 | 1.00 | 65.79 | E |
| ATOM | 17988 | N | VAL | E | 444 | 78.788 | −15.790 | −8.310 | 1.00 | 47.24 | E |
| ATOM | 17989 | CA | VAL | E | 444 | 80.191 | −15.531 | −8.013 | 1.00 | 44.39 | E |
| ATOM | 17990 | CB | VAL | E | 444 | 80.407 | −14.041 | −7.659 | 1.00 | 57.41 | E |
| ATOM | 17991 | CG1 | VAL | E | 444 | 81.882 | −13.685 | −7.742 | 1.00 | 49.71 | E |
| ATOM | 17992 | CG2 | VAL | E | 444 | 79.616 | −13.170 | −8.597 | 1.00 | 49.71 | E |
| ATOM | 17993 | C | VAL | E | 444 | 80.641 | −16.412 | −6.844 | 1.00 | 48.21 | E |
| ATOM | 17994 | O | VAL | E | 444 | 79.885 | −16.633 | −5.901 | 1.00 | 50.47 | E |
| ATOM | 17995 | N | TYR | E | 445 | 81.865 | −16.927 | −6.918 | 1.00 | 42.27 | E |
| ATOM | 17996 | CA | TYR | E | 445 | 82.398 | −17.786 | −5.868 | 1.00 | 41.34 | E |
| ATOM | 17997 | CB | TYR | E | 445 | 82.358 | −19.241 | −6.310 | 1.00 | 73.76 | E |
| ATOM | 17998 | CG | TYR | E | 445 | 80.963 | −19.709 | −6.627 | 1.00 | 46.18 | E |
| ATOM | 17999 | CD1 | TYR | E | 445 | 80.439 | −19.587 | −7.918 | 1.00 | 46.18 | E |
| ATOM | 18000 | CE1 | TYR | E | 445 | 79.139 | −20.035 | −8.214 | 1.00 | 46.18 | E |
| ATOM | 18001 | CD2 | TYR | E | 445 | 80.162 | −20.274 | −5.640 | 1.00 | 46.18 | E |
| ATOM | 18002 | CE2 | TYR | E | 445 | 78.868 | −20.719 | −5.917 | 1.00 | 46.18 | E |
| ATOM | 18003 | CZ | TYR | E | 445 | 78.361 | −20.605 | −7.201 | 1.00 | 46.18 | E |
| ATOM | 18004 | OH | TYR | E | 445 | 77.102 | −21.096 | −7.474 | 1.00 | 46.18 | E |
| ATOM | 18005 | C | TYR | E | 445 | 83.817 | −17.370 | −5.544 | 1.00 | 42.80 | E |
| ATOM | 18006 | O | TYR | E | 445 | 84.597 | −17.089 | −6.452 | 1.00 | 45.66 | E |
| ATOM | 18007 | N | THR | E | 446 | 84.153 | −17.357 | −4.252 | 1.00 | 50.31 | E |
| ATOM | 18008 | CA | THR | E | 446 | 85.472 | −16.917 | −3.813 | 1.00 | 57.29 | E |
| ATOM | 18009 | CB | THR | E | 446 | 85.321 | −15.699 | −2.895 | 1.00 | 55.50 | E |
| ATOM | 18010 | OG1 | THR | E | 446 | 84.471 | −16.046 | −1.798 | 1.00 | 43.65 | E |
| ATOM | 18011 | CG2 | THR | E | 446 | 84.696 | −14.554 | −3.626 | 1.00 | 43.65 | E |
| ATOM | 18012 | C | THR | E | 446 | 86.382 | −17.915 | −3.087 | 1.00 | 67.09 | E |
| ATOM | 18013 | O | THR | E | 446 | 86.256 | −19.131 | −3.231 | 1.00 | 70.76 | E |
| ATOM | 18014 | N | CYS | E | 447 | 87.305 | −17.323 | −2.319 | 1.00 | 92.51 | E |
| ATOM | 18015 | CA | CYS | E | 447 | 88.328 | −17.944 | −1.466 | 1.00 | 93.49 | E |
| ATOM | 18016 | C | CYS | E | 447 | 89.451 | −18.788 | −2.061 | 1.00 | 93.52 | E |
| ATOM | 18017 | O | CYS | E | 447 | 89.265 | −19.557 | −2.998 | 1.00 | 90.38 | E |
| ATOM | 18018 | CB | CYS | E | 447 | 87.676 | −18.709 | −0.302 | 1.00 | 121.93 | E |
| ATOM | 18019 | SG | CYS | E | 447 | 86.565 | −20.077 | −0.738 | 1.00 | 107.46 | E |
| ATOM | 18020 | N | SER | E | 448 | 90.635 | −18.607 | −1.482 | 1.00 | 194.89 | E |
| ATOM | 18021 | CA | SER | E | 448 | 91.840 | −19.315 | −1.887 | 1.00 | 194.89 | E |
| ATOM | 18022 | CB | SER | E | 448 | 92.835 | −18.347 | −2.523 | 1.00 | 200.84 | E |
| ATOM | 18023 | OG | SER | E | 448 | 92.271 | −17.720 | −3.661 | 1.00 | 200.84 | E |
| ATOM | 18024 | C | SER | E | 448 | 92.465 | −19.972 | −0.661 | 1.00 | 194.89 | E |
| ATOM | 18025 | O | SER | E | 448 | 91.767 | −20.250 | 0.315 | 1.00 | 194.89 | E |
| ATOM | 18026 | N | GLU | E | 449 | 93.779 | −20.195 | −0.700 | 1.00 | 150.54 | E |
| ATOM | 18027 | CA | GLU | E | 449 | 94.479 | −20.856 | 0.398 | 1.00 | 150.54 | E |
| ATOM | 18028 | CB | GLU | E | 449 | 94.134 | −20.207 | 1.743 | 1.00 | 121.95 | E |
| ATOM | 18029 | CG | GLU | E | 449 | 94.904 | −18.939 | 2.045 | 1.00 | 121.95 | E |
| ATOM | 18030 | CD | GLU | E | 449 | 94.929 | −17.990 | 0.870 | 1.00 | 121.95 | E |
| ATOM | 18031 | OE1 | GLU | E | 449 | 93.839 | −17.645 | 0.369 | 1.00 | 121.95 | E |
| ATOM | 18032 | OE2 | GLU | E | 449 | 96.038 | −17.591 | 0.449 | 1.00 | 121.95 | E |
| ATOM | 18033 | C | GLU | E | 449 | 93.998 | −22.297 | 0.395 | 1.00 | 150.54 | E |
| ATOM | 18034 | O | GLU | E | 449 | 94.781 | −23.235 | 0.536 | 1.00 | 150.54 | E |
| ATOM | 18035 | N | GLY | E | 450 | 92.692 | −22.445 | 0.215 | 1.00 | 86.75 | E |
| ATOM | 18036 | CA | GLY | E | 450 | 92.046 | −23.741 | 0.186 | 1.00 | 86.75 | E |
| ATOM | 18037 | C | GLY | E | 450 | 90.669 | −23.523 | 0.769 | 1.00 | 86.75 | E |
| ATOM | 18038 | O | GLY | E | 450 | 89.947 | −24.471 | 1.087 | 1.00 | 86.75 | E |
| ATOM | 18039 | N | CYS | E | 451 | 90.311 | −22.250 | 0.900 | 1.00 | 162.80 | E |
| ATOM | 18040 | CA | CYS | E | 451 | 89.030 | −21.859 | 1.462 | 1.00 | 162.80 | E |
| ATOM | 18041 | CB | CYS | E | 451 | 87.889 | −22.402 | 0.625 | 1.00 | 166.53 | E |
| ATOM | 18042 | SG | CYS | E | 451 | 87.817 | −21.639 | −1.009 | 1.00 | 166.53 | E |
| ATOM | 18043 | C | CYS | E | 451 | 88.918 | −22.408 | 2.862 | 1.00 | 162.80 | E |
| ATOM | 18044 | O | CYS | E | 451 | 87.936 | −23.134 | 3.122 | 1.00 | 162.80 | E |
| ATOM | 18045 | OXT | CYS | E | 451 | 89.816 | −22.105 | 3.672 | 1.00 | 138.90 | E |
| ATOM | 18046 | CB | ARG | F | 1 | 71.762 | 17.988 | −13.763 | 1.00 | 160.20 | F |
| ATOM | 18047 | CG | ARG | F | 1 | 70.244 | 18.165 | −13.581 | 1.00 | 160.20 | F |
| ATOM | 18048 | CD | ARG | F | 1 | 69.857 | 19.307 | −12.635 | 1.00 | 160.20 | F |
| ATOM | 18049 | NE | ARG | F | 1 | 69.629 | 20.576 | −13.331 | 1.00 | 160.20 | F |
| ATOM | 18050 | CZ | ARG | F | 1 | 68.652 | 20.794 | −14.212 | 1.00 | 160.20 | F |
| ATOM | 16051 | NH1 | ARG | F | 1 | 67.794 | 19.830 | −14.519 | 1.00 | 160.20 | F |
| ATOM | 18052 | NH2 | ARG | F | 1 | 68.534 | 21.981 | −14.794 | 1.00 | 160.20 | F |
| ATOM | 18053 | C | ARG | F | 1 | 71.879 | 19.602 | −15.693 | 1.00 | 200.84 | F |
| ATOM | 18054 | O | ARG | F | 1 | 70.799 | 20.191 | −15.713 | 1.00 | 200.84 | F |
| ATOM | 18055 | N | ARG | F | 1 | 72.536 | 20.313 | −13.398 | 1.00 | 200.84 | F |
| ATOM | 18056 | CA | ARG | F | 1 | 72.519 | 19.181 | −14.367 | 1.00 | 200.84 | F |
| ATOM | 18057 | N | THR | F | 2 | 72.554 | 19.304 | −16.800 | 1.00 | 200.81 | F |
| ATOM | 18058 | CA | THR | F | 2 | 72.051 | 19.658 | −18.127 | 1.00 | 200.81 | F |
| ATOM | 18059 | CB | THR | F | 2 | 73.181 | 19.609 | −19.190 | 1.00 | 133.73 | F |
| ATOM | 18060 | OG1 | THR | F | 2 | 74.264 | 20.456 | −18.787 | 1.00 | 132.47 | F |
| ATOM | 18061 | CG2 | THR | F | 2 | 72.655 | 20.060 | −20.556 | 1.00 | 135.62 | F |
| ATOM | 18062 | C | THR | F | 2 | 70.955 | 18.700 | −18.588 | 1.00 | 200.81 | F |
| ATOM | 18063 | O | THR | F | 2 | 71.236 | 17.531 | −18.860 | 1.00 | 200.81 | F |
| ATOM | 18064 | N | PRO | F | 3 | 69.690 | 19.166 | −18.669 | 1.00 | 200.84 | F |
| ATOM | 18065 | CD | PRO | F | 3 | 69.056 | 20.438 | −18.268 | 1.00 | 85.85 | F |
| ATOM | 18066 | CA | PRO | F | 3 | 68.684 | 18.205 | −19.127 | 1.00 | 200.84 | F |
| ATOM | 18067 | CB | PRO | F | 3 | 67.463 | 19.088 | −19.391 | 1.00 | 84.09 | F |
| ATOM | 18068 | CG | PRO | F | 3 | 67.556 | 20.083 | −18.271 | 1.00 | 83.96 | F |
| ATOM | 18069 | C | PRO | F | 3 | 69.225 | 17.501 | −20.375 | 1.00 | 200.84 | F |
| ATOM | 18070 | O | PRO | F | 3 | 68.976 | 16.312 | −20.580 | 1.00 | 200.84 | F |
| ATOM | 18071 | N | SER | F | 4 | 69.987 | 18.246 | −21.181 | 1.00 | 121.50 | F |
| ATOM | 18072 | CA | SER | F | 4 | 70.624 | 17.720 | −22.390 | 1.00 | 118.07 | F |
| ATOM | 18073 | CB | SER | F | 4 | 69.731 | 16.694 | −23.099 | 1.00 | 124.32 | F |
| ATOM | 18074 | OG | SER | F | 4 | 68.771 | 17.326 | −23.925 | 1.00 | 126.27 | F |
| ATOM | 18075 | C | SER | F | 4 | 71.017 | 18.784 | −23.406 | 1.00 | 118.41 | F |
| ATOM | 18076 | O | SER | F | 4 | 72.009 | 18.612 | −24.116 | 1.00 | 118.56 | F |
| ATOM | 18077 | N | ASP | F | 5 | 70.241 | 19.868 | −23.469 | 1.00 | 76.84 | F |
| ATOM | 18078 | CA | ASP | F | 5 | 70.462 | 20.964 | −24.423 | 1.00 | 76.95 | F |
| ATOM | 18079 | CB | ASP | F | 5 | 71.867 | 20.865 | −25.039 | 1.00 | 126.80 | F |
| ATOM | 18080 | CG | ASP | F | 5 | 72.138 | 21.930 | −26.077 | 1.00 | 126.80 | F |
| ATOM | 18081 | OD1 | ASP | F | 5 | 73.174 | 21.824 | −26.767 | 1.00 | 126.80 | F |
| ATOM | 18082 | OD2 | ASP | F | 5 | 71.330 | 22.872 | −26.203 | 1.00 | 125.36 | F |
| ATOM | 18083 | C | ASP | F | 5 | 69.389 | 20.902 | −25.536 | 1.00 | 74.45 | F |
| ATOM | 18084 | O | ASP | F | 5 | 69.100 | 21.903 | −26.195 | 1.00 | 77.96 | F |
| ATOM | 18085 | N | LYS | F | 6 | 68.798 | 19.720 | −25.724 | 1.00 | 54.61 | F |
| ATOM | 18086 | CA | LYS | F | 6 | 67.764 | 19.482 | −26.742 | 1.00 | 50.99 | F |
| ATOM | 18087 | CB | LYS | F | 6 | 67.539 | 17.990 | −26.951 | 1.00 | 59.64 | F |
| ATOM | 18088 | CG | LYS | F | 6 | 68.631 | 17.196 | −27.623 | 1.00 | 66.05 | F |
| ATOM | 18089 | CD | LYS | F | 6 | 68.248 | 15.714 | −27.574 | 1.00 | 69.66 | F |
| ATOM | 18090 | CE | LYS | F | 6 | 69.157 | 14.855 | −28.431 | 1.00 | 72.93 | F |
| ATOM | 18091 | NZ | LYS | F | 6 | 69.028 | 15.213 | −29.870 | 1.00 | 74.12 | F |
| ATOM | 18092 | C | LYS | F | 6 | 66.418 | 20.027 | −26.336 | 1.00 | 46.32 | F |
| ATOM | 18093 | O | LYS | F | 6 | 65.988 | 19.808 | −25.210 | 1.00 | 50.20 | F |
| ATOM | 18094 | N | PRO | F | 7 | 65.726 | 20.748 | −27.238 | 1.00 | 43.47 | F |
| ATOM | 18095 | CD | PRO | F | 7 | 66.111 | 21.348 | −28.522 | 1.00 | 35.30 | F |
| ATOM | 18096 | CA | PRO | F | 7 | 64.412 | 21.244 | −26.822 | 1.00 | 41.51 | F |
| ATOM | 18097 | CB | PRO | F | 7 | 63.946 | 22.070 | −28.024 | 1.00 | 37.30 | F |
| ATOM | 18098 | CG | PRO | F | 7 | 64.767 | 21.589 | −29.148 | 1.00 | 36.17 | F |
| ATOM | 18099 | C | PRO | F | 7 | 63.533 | 20.023 | −26.543 | 1.00 | 39.57 | F |
| ATOM | 18100 | O | PRO | F | 7 | 63.497 | 19.076 | −27.319 | 1.00 | 40.58 | F |
| ATOM | 18101 | N | VAL | F | 8 | 62.844 | 20.037 | −25.416 | 1.00 | 35.87 | F |
| ATOM | 18102 | CA | VAL | F | 8 | 62.026 | 18.902 | −25.038 | 1.00 | 37.03 | F |
| ATOM | 18103 | CB | VAL | F | 8 | 62.879 | 17.864 | −24.249 | 1.00 | 37.19 | F |
| ATOM | 18104 | CG1 | VAL | F | 8 | 63.510 | 18.534 | −23.034 | 1.00 | 37.77 | F |
| ATOM | 18105 | CG2 | VAL | F | 8 | 62.024 | 16.669 | −23.822 | 1.00 | 39.89 | F |
| ATOM | 18106 | C | VAL | F | 8 | 60.868 | 19.336 | −24.163 | 1.00 | 39.53 | F |
| ATOM | 18107 | O | VAL | F | 8 | 60.882 | 20.426 | −23.574 | 1.00 | 38.77 | F |
| ATOM | 18108 | N | ALA | F | 9 | 59.867 | 18.476 | −24.060 | 1.00 | 36.71 | F |
| ATOM | 18109 | CA | ALA | F | 9 | 58.725 | 18.794 | −23.230 | 1.00 | 42.95 | F |
| ATOM | 18110 | CB | ALA | F | 9 | 57.915 | 19.920 | −23.858 | 1.00 | 39.82 | F |
| ATOM | 18111 | C | ALA | F | 9 | 57.853 | 17.583 | −23.033 | 1.00 | 43.93 | F |
| ATOM | 18112 | O | ALA | F | 9 | 57.608 | 16.815 | −23.960 | 1.00 | 41.91 | F |
| ATOM | 18113 | N | HIS | F | 10 | 57.403 | 17.402 | −21.806 | 1.00 | 33.65 | F |
| ATOM | 18114 | CA | HIS | F | 10 | 56.514 | 16.307 | −21.485 | 1.00 | 34.52 | F |
| ATOM | 18115 | CB | HIS | F | 10 | 57.254 | 15.147 | −20.813 | 1.00 | 30.10 | F |
| ATOM | 18116 | CG | HIS | F | 10 | 56.363 | 13.988 | −20.478 | 1.00 | 31.59 | F |
| ATOM | 18117 | CD2 | HIS | F | 10 | 55.753 | 13.633 | −19.319 | 1.00 | 29.32 | F |
| ATOM | 18118 | ND1 | HIS | F | 10 | 55.946 | 13.073 | −21.425 | 1.00 | 31.73 | F |
| ATOM | 18119 | CE1 | HIS | F | 10 | 55.117 | 12.208 | −20.862 | 1.00 | 32.08 | F |
| ATOM | 18120 | NE2 | HIS | F | 10 | 54.983 | 12.526 | −19.586 | 1.00 | 29.56 | F |
| ATOM | 18121 | C | HIS | F | 10 | 55.538 | 16.933 | −20.508 | 1.00 | 32.73 | F |
| ATOM | 18122 | O | HIS | F | 10 | 55.828 | 17.068 | −19.315 | 1.00 | 30.89 | F |
| ATOM | 18123 | N | VAL | F | 11 | 54.391 | 17.350 | −21.017 | 1.00 | 52.77 | F |
| ATOM | 18124 | CA | VAL | F | 11 | 53.408 | 17.981 | −20.164 | 1.00 | 55.35 | F |
| ATOM | 18125 | CB | VAL | F | 11 | 52.805 | 19.233 | −20.832 | 1.00 | 52.25 | F |
| ATOM | 18126 | CG1 | VAL | F | 11 | 53.910 | 20.244 | −21.080 | 1.00 | 48.31 | F |
| ATOM | 18127 | CG2 | VAL | F | 11 | 52.111 | 18.862 | −22.138 | 1.00 | 54.23 | F |
| ATOM | 18128 | C | VAL | F | 11 | 52.331 | 16.985 | −19.830 | 1.00 | 53.96 | F |
| ATOM | 18129 | O | VAL | F | 11 | 52.099 | 16.034 | −20.577 | 1.00 | 50.97 | F |
| ATOM | 18130 | N | VAL | F | 12 | 51.679 | 17.206 | −18.697 | 1.00 | 55.75 | F |
| ATOM | 18131 | CA | VAL | F | 12 | 50.652 | 16.297 | −18.240 | 1.00 | 53.92 | F |
| ATOM | 18132 | CB | VAL | F | 12 | 51.149 | 15.593 | −16.976 | 1.00 | 47.25 | F |
| ATOM | 18133 | CG1 | VAL | F | 12 | 51.113 | 16.562 | −15.801 | 1.00 | 41.05 | F |
| ATOM | 18134 | CG2 | VAL | F | 12 | 50.347 | 14.349 | −16.723 | 1.00 | 50.72 | F |
| ATOM | 18135 | C | VAL | F | 12 | 49.352 | 17.057 | −17.983 | 1.00 | 51.88 | F |
| ATOM | 18136 | O | VAL | F | 12 | 49.360 | 18.278 | −17.847 | 1.00 | 51.72 | F |
| ATOM | 18137 | N | ALA | F | 13 | 48.241 | 16.331 | −17.935 | 1.00 | 48.72 | F |
| ATOM | 18138 | CA | ALA | F | 13 | 46.923 | 16.929 | −17.722 | 1.00 | 48.39 | F |
| ATOM | 18139 | CB | ALA | F | 13 | 45.827 | 15.946 | −18.118 | 1.00 | 26.20 | F |
| ATOM | 18140 | C | ALA | F | 13 | 46.687 | 17.389 | −16.295 | 1.00 | 52.37 | F |
| ATOM | 18141 | O | ALA | F | 13 | 47.063 | 16.700 | −15.341 | 1.00 | 49.88 | F |
| ATOM | 18142 | N | ASN | F | 14 | 46.047 | 18.554 | −16.167 | 1.00 | 49.44 | F |
| ATOM | 18143 | CA | ASN | F | 14 | 45.717 | 19.153 | −14.872 | 1.00 | 50.56 | F |
| ATOM | 18144 | CB | ASN | F | 14 | 45.518 | 20.649 | −15.046 | 1.00 | 65.25 | F |
| ATOM | 18145 | CG | ASN | F | 14 | 45.190 | 21.343 | −13.749 | 1.00 | 67.24 | F |
| ATOM | 18146 | OD1 | ASN | F | 14 | 44.928 | 22.546 | −13.733 | 1.00 | 66.82 | F |
| ATOM | 18147 | ND2 | ASN | F | 14 | 45.208 | 20.595 | −12.650 | 1.00 | 69.36 | F |
| ATOM | 18148 | C | ASN | F | 14 | 44.434 | 18.533 | −14.328 | 1.00 | 53.44 | F |
| ATOM | 18149 | O | ASN | F | 14 | 43.362 | 18.718 | −14.896 | 1.00 | 53.96 | F |
| ATOM | 18150 | N | PRO | F | 15 | 44.523 | 17.799 | −13.210 | 1.00 | 56.59 | F |
| ATOM | 18151 | CD | PRO | F | 15 | 45.710 | 17.511 | −12.387 | 1.00 | 52.72 | F |
| ATOM | 18152 | CA | PRO | F | 15 | 43.327 | 17.166 | −12.639 | 1.00 | 60.19 | F |
| ATOM | 18153 | CB | PRO | F | 15 | 43.898 | 16.171 | −11.619 | 1.00 | 57.06 | F |
| ATOM | 18154 | CG | PRO | F | 15 | 45.404 | 16.129 | −11.903 | 1.00 | 55.33 | F |
| ATOM | 18155 | C | PRO | F | 15 | 42.391 | 18.166 | −11.975 | 1.00 | 61.40 | F |
| ATOM | 18156 | O | PRO | F | 15 | 41.202 | 17.894 | −11.805 | 1.00 | 63.43 | F |
| ATOM | 18157 | N | GLN | F | 16 | 42.940 | 19.320 | −11.606 | 1.00 | 112.68 | F |
| ATOM | 18158 | CA | GLN | F | 16 | 42.180 | 20.372 | −10.938 | 1.00 | 111.63 | F |
| ATOM | 18159 | CB | GLN | F | 16 | 43.149 | 21.308 | −10.197 | 1.00 | 126.71 | F |
| ATOM | 18160 | CG | GLN | F | 16 | 42.524 | 22.142 | −9.084 | 1.00 | 129.64 | F |
| ATOM | 18161 | CD | GLN | F | 16 | 41.902 | 21.286 | −7.997 | 1.00 | 131.43 | F |
| ATOM | 18162 | OE1 | GLN | F | 16 | 42.484 | 20.288 | −7.572 | 1.00 | 133.55 | F |
| ATOM | 18163 | NE2 | GLN | F | 16 | 40.718 | 21.679 | −7.532 | 1.00 | 130.08 | F |
| ATOM | 18164 | C | GLN | F | 16 | 41.312 | 21.177 | −11.915 | 1.00 | 108.30 | F |
| ATOM | 18165 | O | GLN | F | 16 | 40.641 | 22.130 | −11.520 | 1.00 | 108.87 | F |
| ATOM | 18166 | N | ALA | F | 17 | 41.324 | 20.794 | −13.187 | 1.00 | 113.15 | F |
| ATOM | 18167 | CA | ALA | F | 17 | 40.530 | 21.496 | −14.191 | 1.00 | 116.91 | F |
| ATOM | 18168 | CB | ALA | F | 17 | 41.436 | 22.066 | −15.271 | 1.00 | 58.21 | F |
| ATOM | 18169 | C | ALA | F | 17 | 39.504 | 20.561 | −14.811 | 1.00 | 121.41 | F |
| ATOM | 18170 | O | ALA | F | 17 | 39.546 | 20.278 | −16.008 | 1.00 | 123.05 | F |
| ATOM | 18171 | N | GLU | F | 18 | 38.582 | 20.085 | −13.984 | 1.00 | 92.47 | F |
| ATOM | 18172 | CA | GLU | F | 18 | 37.535 | 19.175 | −14.431 | 1.00 | 94.47 | F |
| ATOM | 18173 | CB | GLU | F | 18 | 36.499 | 18.987 | −13.323 | 1.00 | 153.64 | F |
| ATOM | 18174 | CG | GLU | F | 18 | 35.793 | 20.273 | −12.920 | 1.00 | 153.64 | F |
| ATOM | 18175 | CD | GLU | F | 18 | 34.669 | 20.040 | −11.927 | 1.00 | 153.64 | F |
| ATOM | 18176 | OE1 | GLU | F | 18 | 33.712 | 19.314 | −12.271 | 1.00 | 153.64 | F |
| ATOM | 18177 | OE2 | GLU | F | 18 | 34.739 | 20.582 | −10.803 | 1.00 | 153.64 | F |
| ATOM | 18178 | C | GLU | F | 18 | 36.830 | 19.662 | −15.695 | 1.00 | 94.13 | F |
| ATOM | 18179 | O | GLU | F | 18 | 36.696 | 20.868 | −15.932 | 1.00 | 93.74 | F |
| ATOM | 18180 | N | GLY | F | 19 | 36.389 | 18.704 | −16.504 | 1.00 | 84.48 | F |
| ATOM | 18181 | CA | GLY | F | 19 | 35.677 | 19.022 | −17.727 | 1.00 | 81.97 | F |
| ATOM | 18182 | C | GLY | F | 19 | 36.506 | 19.461 | −18.918 | 1.00 | 80.47 | F |
| ATOM | 18183 | O | GLY | F | 19 | 35.978 | 19.588 | −20.021 | 1.00 | 80.48 | F |
| ATOM | 18184 | N | GLN | F | 20 | 37.798 | 19.696 | −18.716 | 1.00 | 86.19 | F |
| ATOM | 18185 | CA | GLN | F | 20 | 38.655 | 20.128 | −19.815 | 1.00 | 81.76 | F |
| ATOM | 18186 | CB | GLN | F | 20 | 38.782 | 21.646 | −19.793 | 1.00 | 103.23 | F |
| ATOM | 18187 | CG | GLN | F | 20 | 39.247 | 22.181 | −18.460 | 1.00 | 104.21 | F |
| ATOM | 18188 | CD | GLN | F | 20 | 39.300 | 23.687 | −18.435 | 1.00 | 103.97 | F |
| ATOM | 18189 | OE1 | GLN | F | 20 | 38.288 | 24.350 | −18.646 | 1.00 | 103.62 | F |
| ATOM | 18190 | NE2 | GLN | F | 20 | 40.483 | 24.240 | −18.178 | 1.00 | 103.42 | F |
| ATOM | 18191 | C | GLN | F | 20 | 40.040 | 19.485 | −19.776 | 1.00 | 76.50 | F |
| ATOM | 18192 | O | GLN | F | 20 | 40.415 | 18.830 | −18.804 | 1.00 | 75.21 | F |
| ATOM | 18193 | N | LEU | F | 21 | 40.796 | 19.670 | −20.848 | 1.00 | 59.49 | F |
| ATOM | 18194 | CA | LEU | F | 21 | 42.134 | 19.107 | −20.935 | 1.00 | 55.95 | F |
| ATOM | 18195 | CB | LEU | F | 21 | 42.262 | 18.229 | −22.188 | 1.00 | 57.25 | F |
| ATOM | 18196 | CG | LEU | F | 21 | 43.624 | 17.591 | −22.495 | 1.00 | 56.03 | F |
| ATOM | 18197 | CD1 | LEU | F | 21 | 44.185 | 16.901 | −21.263 | 1.00 | 53.33 | F |
| ATOM | 18198 | CD2 | LEU | F | 21 | 43.458 | 16.606 | −23.635 | 1.00 | 58.07 | F |
| ATOM | 18199 | C | LEU | F | 21 | 43.163 | 20.216 | −20.980 | 1.00 | 54.56 | F |
| ATOM | 18200 | O | LEU | F | 21 | 43.491 | 20.726 | −22.049 | 1.00 | 49.88 | F |
| ATOM | 18201 | N | GLN | F | 22 | 43.673 | 20.586 | −19.813 | 1.00 | 55.01 | F |
| ATOM | 18202 | CA | GLN | F | 22 | 44.667 | 21.645 | −19.707 | 1.00 | 59.61 | F |
| ATOM | 18203 | CB | GLN | F | 22 | 44.215 | 22.629 | −18.630 | 1.00 | 72.86 | F |
| ATOM | 18204 | CG | GLN | F | 22 | 45.213 | 23.704 | −18.269 | 1.00 | 79.63 | F |
| ATOM | 18205 | CD | GLN | F | 22 | 44.824 | 24.435 | −16.998 | 1.00 | 85.85 | F |
| ATOM | 18206 | OE1 | GLN | F | 22 | 45.587 | 25.243 | −16.475 | 1.00 | 91.08 | F |
| ATOM | 18207 | NE2 | GLN | F | 22 | 43.631 | 24.151 | −16.494 | 1.00 | 86.61 | F |
| ATOM | 18208 | C | GLN | F | 22 | 46.032 | 21.035 | −19.363 | 1.00 | 57.00 | F |
| ATOM | 18209 | O | GLN | F | 22 | 46.158 | 20.275 | −18.400 | 1.00 | 55.95 | F |
| ATOM | 18210 | N | TRP | F | 23 | 47.050 | 21.364 | −20.153 | 1.00 | 54.96 | F |
| ATOM | 18211 | CA | TRP | F | 23 | 48.388 | 20.823 | −19.930 | 1.00 | 48.84 | F |
| ATOM | 18212 | CB | TRP | F | 23 | 49.158 | 20.707 | −21.259 | 1.00 | 39.58 | F |
| ATOM | 18213 | CG | TRP | F | 23 | 48.479 | 19.918 | −22.328 | 1.00 | 40.10 | F |
| ATOM | 18214 | CD2 | TRP | F | 23 | 48.237 | 18.507 | −22.337 | 1.00 | 38.21 | F |
| ATOM | 18215 | CE2 | TRP | F | 23 | 47.560 | 18.208 | −23.539 | 1.00 | 39.83 | F |
| ATOM | 18216 | CE3 | TRP | F | 23 | 48.526 | 17.467 | −21.449 | 1.00 | 37.90 | F |
| ATOM | 18217 | CD1 | TRP | F | 23 | 47.960 | 20.403 | −23.493 | 1.00 | 39.57 | F |
| ATOM | 18218 | NE1 | TRP | F | 23 | 47.408 | 19.382 | −24.226 | 1.00 | 38.66 | F |
| ATOM | 18219 | CZ2 | TRP | F | 23 | 47.164 | 16.913 | −23.874 | 1.00 | 41.89 | F |
| ATOM | 18220 | CZ3 | TRP | F | 23 | 48.130 | 16.172 | −21.784 | 1.00 | 42.34 | F |
| ATOM | 18221 | CH2 | TRP | F | 23 | 47.456 | 15.912 | −22.987 | 1.00 | 40.15 | F |
| ATOM | 18222 | C | TRP | F | 23 | 49.237 | 21.660 | −18.982 | 1.00 | 49.34 | F |
| ATOM | 18223 | O | TAP | F | 23 | 49.232 | 22.884 | −19.058 | 1.00 | 50.62 | F |
| ATOM | 18224 | N | LEU | F | 24 | 49.972 | 20.999 | −18.093 | 1.00 | 45.14 | F |
| ATOM | 18225 | CA | LEU | F | 24 | 50.870 | 21.713 | −17.192 | 1.00 | 42.83 | F |
| ATOM | 18226 | CB | LEU | F | 24 | 50.379 | 21.726 | −15.744 | 1.00 | 61.41 | F |
| ATOM | 18227 | CG | LEU | F | 24 | 48.944 | 21.401 | −15.381 | 1.00 | 62.14 | F |
| ATOM | 18228 | CD1 | LEU | F | 24 | 48.726 | 19.919 | −15.553 | 1.00 | 63.76 | F |
| ATOM | 18229 | CD2 | LEU | F | 24 | 48.693 | 21.801 | −13.944 | 1.00 | 57.25 | F |
| ATOM | 18230 | C | LEU | F | 24 | 52.221 | 21.042 | −17.218 | 1.00 | 49.69 | F |
| ATOM | 18231 | O | LEU | F | 24 | 52.347 | 19.873 | −17.596 | 1.00 | 46.67 | F |
| ATOM | 18232 | N | ASN | F | 25 | 53.231 | 21.790 | −16.805 | 1.00 | 48.39 | F |
| ATOM | 18233 | CA | ASN | F | 25 | 54.581 | 21.275 | −16.759 | 1.00 | 49.83 | F |
| ATOM | 18234 | CB | ASN | F | 25 | 55.457 | 22.071 | −17.708 | 1.00 | 47.01 | F |
| ATOM | 18235 | CG | ASN | F | 25 | 55.733 | 23.451 | −17.194 | 1.00 | 48.58 | F |
| ATOM | 18236 | OD1 | ASN | F | 25 | 56.875 | 23.793 | −16.871 | 1.00 | 52.57 | F |
| ATOM | 18237 | ND2 | ASN | F | 25 | 54.680 | 24.256 | −17.092 | 1.00 | 44.98 | F |
| ATOM | 18238 | C | ASN | F | 25 | 55.138 | 21.404 | −15.333 | 1.00 | 50.36 | F |
| ATOM | 18239 | O | ASN | F | 25 | 56.263 | 20.987 | −15.052 | 1.00 | 52.72 | F |
| ATOM | 18240 | N | ARG | F | 26 | 54.350 | 21.981 | −14.435 | 1.00 | 60.37 | F |
| ATOM | 18241 | CA | ARG | F | 26 | 54.793 | 22.176 | −13.064 | 1.00 | 61.65 | F |
| ATOM | 18242 | CB | ARG | F | 26 | 54.138 | 23.430 | −12.488 | 1.00 | 61.56 | F |
| ATOM | 18243 | CG | ARG | F | 26 | 52.630 | 23.450 | −12.558 | 1.00 | 62.47 | F |
| ATOM | 18244 | CD | ARG | F | 26 | 52.081 | 24.566 | −11.695 | 1.00 | 66.53 | F |
| ATOM | 18245 | NE | ARG | F | 26 | 50.624 | 24.619 | −11.737 | 1.00 | 67.67 | F |
| ATOM | 18246 | CZ | ARG | F | 26 | 49.929 | 25.212 | −12.701 | 1.00 | 68.23 | F |
| ATOM | 18247 | NH1 | ARG | F | 26 | 50.558 | 25.815 | −13.708 | 1.00 | 68.71 | F |
| ATOM | 18248 | NH2 | ARG | F | 26 | 48.605 | 25.196 | −12.663 | 1.00 | 67.26 | F |
| ATOM | 18249 | C | ARG | F | 26 | 54.520 | 20.996 | −12.141 | 1.00 | 59.71 | F |
| ATOM | 18250 | O | ARG | F | 26 | 54.021 | 21.173 | −11.032 | 1.00 | 59.42 | F |
| ATOM | 18251 | N | ARG | F | 27 | 54.854 | 19.792 | −12.584 | 1.00 | 59.08 | F |
| ATOM | 18252 | CA | ARG | F | 27 | 54.613 | 18.623 | −11.760 | 1.00 | 60.01 | F |
| ATOM | 18253 | CB | ARG | F | 27 | 53.383 | 17.861 | −12.259 | 1.00 | 42.73 | F |
| ATOM | 18254 | CG | ARG | F | 27 | 52.103 | 18.604 | −12.068 | 1.00 | 44.15 | F |
| ATOM | 18255 | CD | ARG | F | 27 | 51.980 | 19.049 | −10.630 | 1.00 | 46.52 | F |
| ATOM | 18256 | NE | ARG | F | 27 | 50.803 | 19.887 | −10.401 | 1.00 | 49.91 | F |
| ATOM | 18257 | CZ | ARG | F | 27 | 49.545 | 19.473 | −10.541 | 1.00 | 53.52 | F |
| ATOM | 18258 | NH1 | ARG | F | 27 | 48.538 | 20.314 | −10.309 | 1.00 | 54.39 | F |
| ATOM | 18259 | NH2 | ARG | F | 27 | 49.299 | 18.216 | −10.911 | 1.00 | 51.75 | F |
| ATOM | 18260 | C | ARG | F | 27 | 55.794 | 17.675 | −11.674 | 1.00 | 60.47 | F |
| ATOM | 18261 | O | ARG | F | 27 | 56.819 | 17.869 | −12.333 | 1.00 | 60.61 | F |
| ATOM | 18262 | N | ALA | F | 28 | 55.610 | 16.648 | −10.847 | 1.00 | 82.46 | F |
| ATOM | 18263 | CA | ALA | F | 28 | 56.592 | 15.607 | −10.577 | 1.00 | 81.36 | F |
| ATOM | 18264 | CB | ALA | F | 28 | 55.874 | 14.321 | −10.183 | 1.00 | 110.83 | F |
| ATOM | 18265 | C | ALA | F | 28 | 57.580 | 15.310 | −11.691 | 1.00 | 81.02 | F |
| ATOM | 18266 | O | ALA | F | 28 | 58.679 | 15.854 | −11.721 | 1.00 | 82.04 | F |
| ATOM | 18267 | N | ASN | F | 29 | 57.195 | 14.439 | −12.609 | 1.00 | 49.03 | F |
| ATOM | 18268 | CA | ASN | F | 29 | 58.098 | 14.069 | −13.681 | 1.00 | 48.20 | F |
| ATOM | 18269 | CB | ASN | F | 29 | 58.196 | 12.548 | −13.731 | 1.00 | 53.94 | F |
| ATOM | 18270 | CG | ASN | F | 29 | 58.779 | 11.967 | −12.455 | 1.00 | 54.00 | F |
| ATOM | 18271 | OD1 | ASN | F | 29 | 59.994 | 11.972 | −12.251 | 1.00 | 59.86 | F |
| ATOM | 18272 | ND2 | ASN | F | 29 | 57.912 | 11.482 | −11.580 | 1.00 | 51.01 | F |
| ATOM | 18273 | C | ASN | F | 29 | 57.669 | 14.630 | −15.028 | 1.00 | 46.54 | F |
| ATOM | 18274 | O | ASN | F | 29 | 57.406 | 13.881 | −15.973 | 1.00 | 44.26 | F |
| ATOM | 18275 | N | ALA | F | 30 | 57.606 | 15.955 | −15.106 | 1.00 | 41.05 | F |
| ATOM | 18276 | CA | ALA | F | 30 | 57.207 | 16.626 | −16.330 | 1.00 | 35.87 | F |
| ATOM | 18277 | CB | ALA | F | 30 | 55.994 | 17.503 | −16.071 | 1.00 | 41.29 | F |
| ATOM | 18278 | C | ALA | F | 30 | 58.378 | 17.465 | −16.846 | 1.00 | 37.27 | F |
| ATOM | 18279 | O | ALA | F | 30 | 59.369 | 17.675 | −16.130 | 1.00 | 41.42 | F |
| ATOM | 18280 | N | LEU | F | 31 | 58.263 | 17.940 | −18.083 | 1.00 | 50.52 | F |
| ATOM | 18281 | CA | LEU | F | 31 | 59.327 | 18.721 | −18.685 | 1.00 | 49.99 | F |
| ATOM | 18282 | CB | LEU | F | 31 | 60.250 | 17.810 | −19.487 | 1.00 | 43.34 | F |
| ATOM | 18283 | CG | LEU | F | 31 | 61.200 | 16.846 | −18.798 | 1.00 | 46.22 | F |
| ATOM | 18284 | CD1 | LEU | F | 31 | 61.739 | 15.852 | −19.813 | 1.00 | 43.84 | F |
| ATOM | 18285 | CD2 | LEU | F | 31 | 62.316 | 17.628 | −18.166 | 1.00 | 45.69 | F |
| ATOM | 18286 | C | LEU | F | 31 | 58.834 | 19.812 | −19.618 | 1.00 | 49.25 | F |
| ATOM | 18287 | O | LEU | F | 31 | 57.795 | 19.673 | −20.269 | 1.00 | 50.06 | F |
| ATOM | 18288 | N | LEU | F | 32 | 59.621 | 20.882 | −19.690 | 1.00 | 41.88 | F |
| ATOM | 18289 | CA | LEU | F | 32 | 59.359 | 22.029 | −20.552 | 1.00 | 41.67 | F |
| ATOM | 18290 | CB | LEU | F | 32 | 58.231 | 22.906 | −19.994 | 1.00 | 44.13 | F |
| ATOM | 18291 | CG | LEU | F | 32 | 57.885 | 24.049 | −20.955 | 1.00 | 42.71 | F |
| ATOM | 18292 | CD1 | LEU | F | 32 | 57.274 | 23.471 | −22.224 | 1.00 | 41.69 | F |
| ATOM | 18293 | CD2 | LEU | F | 32 | 56.927 | 25.019 | −20.292 | 1.00 | 43.03 | F |
| ATOM | 18294 | C | LEU | F | 32 | 60.671 | 22.809 | −20.560 | 1.00 | 41.31 | F |
| ATOM | 18295 | O | LEU | F | 32 | 60.832 | 23.793 | −19.834 | 1.00 | 47.18 | F |
| ATOM | 18296 | N | ALA | F | 33 | 61.605 | 22.374 | −21.396 | 1.00 | 39.67 | F |
| ATOM | 18297 | CA | ALA | F | 33 | 62.911 | 23.010 | −21.419 | 1.00 | 40.19 | F |
| ATOM | 18298 | CB | ALA | F | 33 | 63.870 | 22.174 | −20.608 | 1.00 | 24.54 | F |
| ATOM | 18299 | C | ALA | F | 33 | 63.553 | 23.313 | −22.766 | 1.00 | 41.98 | F |
| ATOM | 18300 | O | ALA | F | 33 | 63.075 | 22.916 | −23.839 | 1.00 | 46.16 | F |
| ATOM | 18301 | N | ASN | F | 34 | 64.671 | 24.021 | −22.667 | 1.00 | 48.57 | F |
| ATOM | 18302 | CA | ASN | F | 34 | 65.468 | 24.388 | −23.812 | 1.00 | 45.76 | F |
| ATOM | 18303 | CB | ASN | F | 34 | 66.202 | 23.161 | −24.325 | 1.00 | 58.85 | F |
| ATOM | 18304 | CG | ASN | F | 34 | 67.080 | 22.554 | −23.273 | 1.00 | 62.37 | F |
| ATOM | 18305 | OD1 | ASN | F | 34 | 67.871 | 23.252 | −22.642 | 1.00 | 65.25 | F |
| ATOM | 18306 | ND2 | ASN | F | 34 | 66.948 | 21.252 | −23.065 | 1.00 | 58.18 | F |
| ATOM | 18307 | C | ASN | F | 34 | 64.747 | 25.075 | −24.954 | 1.00 | 46.51 | F |
| ATOM | 18308 | O | ASN | F | 34 | 65.000 | 24.787 | −26.127 | 1.00 | 49.05 | F |
| ATOM | 18309 | N | GLY | F | 35 | 63.847 | 25.988 | −24.619 | 1.00 | 51.50 | F |
| ATOM | 18310 | CA | GLY | F | 35 | 63.184 | 26.729 | −25.664 | 1.00 | 46.09 | F |
| ATOM | 18311 | C | GLY | F | 35 | 61.732 | 26.460 | −25.947 | 1.00 | 46.44 | F |
| ATOM | 18312 | O | GLY | F | 35 | 61.005 | 27.393 | −26.308 | 1.00 | 47.93 | F |
| ATOM | 18313 | N | VAL | F | 36 | 61.280 | 25.218 | −25.815 | 1.00 | 43.65 | F |
| ATOM | 18314 | CA | VAL | F | 36 | 59.880 | 24.995 | −26.126 | 1.00 | 44.98 | F |
| ATOM | 18315 | CB | VAL | F | 36 | 59.552 | 23.480 | −26.297 | 1.00 | 47.41 | F |
| ATOM | 18316 | CG1 | VAL | F | 36 | 60.724 | 22.651 | −25.882 | 1.00 | 45.44 | F |
| ATOM | 18317 | CG2 | VAL | F | 36 | 58.294 | 23.111 | −25.532 | 1.00 | 47.81 | F |
| ATOM | 18318 | C | VAL | F | 36 | 59.012 | 25.678 | −25.073 | 1.00 | 49.13 | F |
| ATOM | 18319 | O | VAL | F | 36 | 59.317 | 25.651 | −23.881 | 1.00 | 50.09 | F |
| ATOM | 18320 | N | GLU | F | 37 | 57.945 | 26.322 | −25.542 | 1.00 | 47.27 | F |
| ATOM | 18321 | CA | GLU | F | 37 | 57.032 | 27.044 | −24.667 | 1.00 | 49.87 | F |
| ATOM | 18322 | CB | GLU | F | 37 | 56.940 | 28.498 | −25.116 | 1.00 | 61.00 | F |
| ATOM | 18323 | CG | GLU | F | 37 | 58.264 | 29.031 | −25.630 | 1.00 | 68.03 | F |
| ATOM | 18324 | CD | GLU | F | 37 | 58.130 | 30.334 | −26.390 | 1.00 | 71.00 | F |
| ATOM | 18325 | OE1 | GLU | F | 37 | 57.068 | 30.552 | −27.019 | 1.00 | 74.43 | F |
| ATOM | 18326 | OE2 | GLU | F | 37 | 59.095 | 31.131 | −26.375 | 1.00 | 71.56 | F |
| ATOM | 18327 | C | GLU | F | 37 | 55.651 | 26.420 | −24.690 | 1.00 | 48.76 | F |
| ATOM | 18328 | O | GLU | F | 37 | 55.259 | 25.772 | −25.657 | 1.00 | 51.22 | F |
| ATOM | 18329 | N | LEU | F | 38 | 54.931 | 26.600 | −23.595 | 1.00 | 55.07 | F |
| ATOM | 18330 | CA | LEU | F | 38 | 53.581 | 26.095 | −23.486 | 1.00 | 57.11 | F |
| ATOM | 18331 | CB | LEU | F | 38 | 53.391 | 25.367 | −22.161 | 1.00 | 50.78 | F |
| ATOM | 18332 | CG | LEU | F | 38 | 52.017 | 24.714 | −21.964 | 1.00 | 48.69 | F |
| ATOM | 18333 | CD1 | LEU | F | 38 | 51.635 | 23.915 | −23.214 | 1.00 | 47.07 | F |
| ATOM | 18334 | CD2 | LEU | F | 38 | 52.039 | 23.809 | −20.726 | 1.00 | 45.56 | F |
| ATOM | 18335 | C | LEU | F | 38 | 52.755 | 27.365 | −23.528 | 1.00 | 57.38 | F |
| ATOM | 18336 | O | LEU | F | 38 | 52.665 | 28.087 | −22.544 | 1.00 | 50.99 | F |
| ATOM | 18337 | N | ARG | F | 39 | 52.168 | 27.640 | −24.684 | 1.00 | 44.65 | F |
| ATOM | 18338 | CA | ARG | F | 39 | 51.388 | 28.847 | −24.900 | 1.00 | 51.18 | F |
| ATOM | 18339 | CB | ARG | F | 39 | 52.113 | 29.693 | −25.948 | 1.00 | 65.47 | F |
| ATOM | 18340 | CG | ARG | F | 39 | 51.606 | 31.089 | −26.118 | 1.00 | 73.17 | F |
| ATOM | 18341 | CD | ARG | F | 39 | 52.587 | 31.892 | −26.952 | 1.00 | 81.78 | F |
| ATOM | 18342 | NE | ARG | F | 39 | 52.293 | 33.322 | −26.895 | 1.00 | 88.31 | F |
| ATOM | 18343 | CZ | ARG | F | 39 | 53.121 | 34.270 | −27.320 | 1.00 | 89.11 | F |
| ATOM | 18344 | NH1 | ARG | F | 39 | 54.298 | 33.943 | −27.837 | 1.00 | 87.59 | F |
| ATOM | 18345 | NH2 | ARG | F | 39 | 52.778 | 35.547 | −27.219 | 1.00 | 91.41 | F |
| ATOM | 18346 | C | ARG | F | 39 | 49.967 | 28.513 | −25.355 | 1.00 | 53.23 | F |
| ATOM | 18347 | O | ARG | F | 39 | 49.766 | 27.695 | −26.249 | 1.00 | 56.46 | F |
| ATOM | 18348 | N | ASP | F | 40 | 48.981 | 29.139 | −24.729 | 1.00 | 59.11 | F |
| ATOM | 18349 | CA | ASP | F | 40 | 47.586 | 28.910 | −25.083 | 1.00 | 57.06 | F |
| ATOM | 18350 | CB | ASP | F | 40 | 47.250 | 29.617 | −26.404 | 1.00 | 111.99 | F |
| ATOM | 18351 | CG | ASP | F | 40 | 47.576 | 31.108 | −26.376 | 1.00 | 114.13 | F |
| ATOM | 18352 | OD1 | ASP | F | 40 | 47.091 | 31.811 | −25.463 | 1.00 | 111.01 | F |
| ATOM | 18353 | OD2 | ASP | F | 40 | 48.316 | 31.579 | −27.271 | 1.00 | 114.25 | F |
| ATOM | 18354 | C | ASP | F | 40 | 47.272 | 27.418 | −25.197 | 1.00 | 53.19 | F |
| ATOM | 18355 | O | ASP | F | 40 | 46.502 | 27.002 | −26.059 | 1.00 | 52.42 | F |
| ATOM | 18356 | N | ASN | F | 41 | 47.883 | 26.626 | −24.322 | 1.00 | 48.65 | F |
| ATOM | 18357 | CA | ASN | F | 41 | 47.685 | 25.175 | −24.251 | 1.00 | 44.40 | F |
| ATOM | 18358 | CB | ASN | F | 41 | 46.200 | 24.840 | −24.139 | 1.00 | 59.02 | F |
| ATOM | 18359 | CG | ASN | F | 41 | 45.956 | 23.514 | −23.434 | 1.00 | 53.71 | F |
| ATOM | 18360 | OD1 | ASN | F | 41 | 46.446 | 23.285 | −22.322 | 1.00 | 55.05 | F |
| ATOM | 18361 | ND2 | ASN | F | 41 | 45.190 | 22.638 | −24.071 | 1.00 | 49.81 | F |
| ATOM | 18362 | C | ASN | F | 41 | 48.288 | 24.405 | −25.413 | 1.00 | 47.71 | F |
| ATOM | 18363 | O | ASN | F | 41 | 47.856 | 23.290 | −25.731 | 1.00 | 49.22 | F |
| ATOM | 18364 | N | GLN | F | 42 | 49.295 | 24.997 | −26.042 | 1.00 | 54.08 | F |
| ATOM | 18365 | CA | GLN | F | 42 | 49.959 | 24.360 | −27.163 | 1.00 | 52.00 | F |
| ATOM | 18366 | CB | GLN | F | 42 | 49.586 | 25.064 | −28.457 | 1.00 | 47.36 | F |
| ATOM | 18367 | CG | GLN | F | 42 | 48.098 | 25.293 | −28.613 | 1.00 | 49.45 | F |
| ATOM | 18368 | CD | GLN | F | 42 | 47.753 | 26.020 | −29.901 | 1.00 | 48.91 | F |
| ATOM | 18369 | OE1 | GLN | F | 42 | 48.364 | 27.041 | −30.237 | 1.00 | 48.34 | F |
| ATOM | 18370 | NE2 | GLN | F | 42 | 46.760 | 25.503 | −30.626 | 1.00 | 50.67 | F |
| ATOM | 18371 | C | GLN | F | 42 | 51.459 | 24.444 | −26.968 | 1.00 | 50.89 | F |
| ATOM | 18372 | O | GLN | F | 42 | 51.957 | 25.395 | −26.375 | 1.00 | 51.23 | F |
| ATOM | 18373 | N | LEU | F | 43 | 52.182 | 23.446 | −27.458 | 1.00 | 45.10 | F |
| ATOM | 18374 | CA | LEU | F | 43 | 53.624 | 23.457 | −27.337 | 1.00 | 43.68 | F |
| ATOM | 18375 | CB | LEU | F | 43 | 54.164 | 22.031 | −27.334 | 1.00 | 31.66 | F |
| ATOM | 18376 | CG | LEU | F | 43 | 53.721 | 21.215 | −26.117 | 1.00 | 37.82 | F |
| ATOM | 18377 | CD1 | LEU | F | 43 | 54.125 | 19.752 | −26.278 | 1.00 | 37.91 | F |
| ATOM | 18378 | CD2 | LEU | F | 43 | 54.330 | 21.811 | −24.872 | 1.00 | 34.21 | F |
| ATOM | 18379 | C | LEU | F | 43 | 54.153 | 24.221 | −28.531 | 1.00 | 45.70 | F |
| ATOM | 18380 | O | LEU | F | 43 | 53.657 | 24.052 | −29.638 | 1.00 | 42.32 | F |
| ATOM | 18381 | N | VAL | F | 44 | 55.147 | 25.072 | −28.319 | 1.00 | 37.50 | F |
| ATOM | 18382 | CA | VAL | F | 44 | 55.695 | 25.837 | −29.422 | 1.00 | 37.18 | F |
| ATOM | 18383 | CB | VAL | F | 44 | 55.527 | 27.343 | −29.184 | 1.00 | 31.94 | F |
| ATOM | 18384 | CG1 | VAL | F | 44 | 55.942 | 28.121 | −30.419 | 1.00 | 33.13 | F |
| ATOM | 18385 | CG2 | VAL | F | 44 | 54.085 | 27.644 | −28.825 | 1.00 | 26.55 | F |
| ATOM | 18386 | C | VAL | F | 44 | 57.168 | 25.520 | −29.644 | 1.00 | 36.19 | F |
| ATOM | 18387 | O | VAL | F | 44 | 58.009 | 25.730 | −28.764 | 1.00 | 37.23 | F |
| ATOM | 18388 | N | VAL | F | 45 | 57.471 | 25.015 | −30.835 | 1.00 | 36.64 | F |
| ATOM | 18389 | CA | VAL | F | 45 | 58.824 | 24.641 | −31.207 | 1.00 | 42.38 | F |
| ATOM | 18390 | CB | VAL | F | 45 | 58.795 | 23.814 | −32.506 | 1.00 | 72.25 | F |
| ATOM | 18391 | CG1 | VAL | F | 45 | 60.193 | 23.654 | −33.063 | 1.00 | 74.82 | F |
| ATOM | 18392 | CG2 | VAL | F | 45 | 58.189 | 22.453 | −32.224 | 1.00 | 75.65 | F |
| ATOM | 18393 | C | VAL | F | 45 | 59.747 | 25.857 | −31.365 | 1.00 | 44.17 | F |
| ATOM | 18394 | O | VAL | F | 45 | 59.397 | 26.843 | −31.999 | 1.00 | 43.62 | F |
| ATOM | 18395 | N | PRO | F | 46 | 60.950 | 25.790 | −30.781 | 1.00 | 51.20 | F |
| ATOM | 18396 | CD | PRO | F | 46 | 61.354 | 24.800 | −29.762 | 1.00 | 35.54 | F |
| ATOM | 18397 | CA | PRO | F | 46 | 61.926 | 26.881 | −30.850 | 1.00 | 50.05 | F |
| ATOM | 18398 | CB | PRO | F | 46 | 62.626 | 26.783 | −29.512 | 1.00 | 34.90 | F |
| ATOM | 18399 | CG | PRO | F | 46 | 62.734 | 25.305 | −29.342 | 1.00 | 35.60 | F |
| ATOM | 18400 | C | PRO | F | 46 | 62.931 | 26.839 | −31.987 | 1.00 | 53.49 | F |
| ATOM | 18401 | O | PRO | F | 46 | 63.714 | 27.773 | −32.134 | 1.00 | 55.95 | F |
| ATOM | 18402 | N | SER | F | 47 | 62.938 | 25.768 | −32.780 | 1.00 | 65.54 | F |
| ATOM | 18403 | CA | SER | F | 47 | 63.905 | 25.678 | −33.883 | 1.00 | 62.66 | F |
| ATOM | 18404 | CB | SER | F | 47 | 65.292 | 25.372 | −33.324 | 1.00 | 63.49 | F |
| ATOM | 18405 | OG | SER | F | 47 | 65.287 | 24.131 | −32.638 | 1.00 | 64.70 | F |
| ATOM | 18406 | C | SER | F | 47 | 63.567 | 24.645 | −34.956 | 1.00 | 59.35 | F |
| ATOM | 18407 | O | SER | F | 47 | 62.820 | 23.699 | −34.706 | 1.00 | 56.49 | F |
| ATOM | 18408 | N | GLU | F | 48 | 64.125 | 24.848 | −36.151 | 1.00 | 51.94 | F |
| ATOM | 18409 | CA | GLU | F | 48 | 63.926 | 23.949 | −37.289 | 1.00 | 56.43 | F |
| ATOM | 18410 | CB | GLU | F | 48 | 64.806 | 24.366 | −38.469 | 1.00 | 124.88 | F |
| ATOM | 18411 | CG | GLU | F | 48 | 64.630 | 25.795 | −38.959 | 1.00 | 129.62 | F |
| ATOM | 18412 | CD | GLU | F | 48 | 63.458 | 25.963 | −39.908 | 1.00 | 133.97 | F |
| ATOM | 18413 | OE1 | GLU | F | 48 | 62.300 | 25.847 | −39.457 | 1.00 | 135.71 | F |
| ATOM | 18414 | OE2 | GLU | F | 48 | 63.700 | 26.208 | −41.110 | 1.00 | 131.89 | F |
| ATOM | 18415 | C | GLU | F | 48 | 64.391 | 22.585 | −36.815 | 1.00 | 55.02 | F |
| ATOM | 18416 | O | GLU | F | 48 | 65.262 | 22.497 | −35.953 | 1.00 | 60.81 | F |
| ATOM | 18417 | N | GLY | F | 49 | 63.821 | 21.517 | −37.357 | 1.00 | 47.23 | F |
| ATOM | 18418 | CA | GLY | F | 49 | 64.270 | 20.204 | −36.949 | 1.00 | 44.89 | F |
| ATOM | 18419 | C | GLY | F | 49 | 63.280 | 19.065 | −36.981 | 1.00 | 46.90 | F |
| ATOM | 18420 | O | GLY | F | 49 | 62.090 | 19.246 | −37.219 | 1.00 | 49.74 | F |
| ATOM | 18421 | N | LEU | F | 50 | 63.812 | 17.873 | −36.747 | 1.00 | 52.76 | F |
| ATOM | 18422 | CA | LEU | F | 50 | 63.043 | 16.646 | −36.701 | 1.00 | 53.96 | F |
| ATOM | 18423 | CB | LEU | F | 50 | 63.942 | 15.500 | −37.119 | 1.00 | 40.86 | F |
| ATOM | 18424 | CG | LEU | F | 50 | 63.666 | 14.744 | −38.415 | 1.00 | 41.99 | F |
| ATOM | 18425 | CD1 | LEU | F | 50 | 62.991 | 15.624 | −39.425 | 1.00 | 38.80 | F |
| ATOM | 18426 | CD2 | LEU | F | 50 | 64.981 | 14.201 | −38.958 | 1.00 | 42.38 | F |
| ATOM | 18427 | C | LEU | F | 50 | 62.604 | 16.471 | −35.247 | 1.00 | 50.34 | F |
| ATOM | 18428 | O | LEU | F | 50 | 63.421 | 16.568 | −34.332 | 1.00 | 49.06 | F |
| ATOM | 18429 | N | TYR | F | 51 | 61.318 | 16.227 | −35.020 | 1.00 | 48.02 | F |
| ATOM | 18430 | CA | TYR | F | 51 | 60.823 | 16.059 | −33.657 | 1.00 | 46.67 | F |
| ATOM | 18431 | CB | TYR | F | 51 | 59.942 | 17.243 | −33.230 | 1.00 | 36.96 | F |
| ATOM | 18432 | CG | TYR | F | 51 | 60.658 | 18.537 | −32.951 | 1.00 | 37.06 | F |
| ATOM | 18433 | CD1 | TYR | F | 51 | 61.170 | 19.318 | −33.988 | 1.00 | 37.92 | F |
| ATOM | 18434 | CE1 | TYR | F | 51 | 61.816 | 20.518 | −33.734 | 1.00 | 37.48 | F |
| ATOM | 18435 | CD2 | TYR | F | 51 | 60.813 | 18.989 | −31.649 | 1.00 | 38.62 | F |
| ATOM | 18436 | CE2 | TYR | F | 51 | 61.462 | 20.191 | −31.378 | 1.00 | 36.89 | F |
| ATOM | 18437 | CZ | TYR | F | 51 | 61.962 | 20.954 | −32.424 | 1.00 | 36.59 | F |
| ATOM | 18438 | OH | TYR | F | 51 | 62.617 | 22.148 | −32.149 | 1.00 | 36.82 | F |
| ATOM | 18439 | C | TYR | F | 51 | 59.992 | 14.809 | −33.467 | 1.00 | 46.85 | F |
| ATOM | 18440 | O | TYR | F | 51 | 59.229 | 14.412 | −34.351 | 1.00 | 47.74 | F |
| ATOM | 18441 | N | LEU | F | 52 | 60.133 | 14.190 | −32.302 | 1.00 | 40.23 | F |
| ATOM | 18442 | CA | LEU | F | 52 | 59.327 | 13.028 | −31.978 | 1.00 | 41.88 | F |
| ATOM | 18443 | CB | LEU | F | 52 | 60.108 | 12.038 | −31.120 | 1.00 | 58.50 | F |
| ATOM | 18444 | CG | LEU | F | 52 | 59.275 | 10.917 | −30.491 | 1.00 | 54.39 | F |
| ATOM | 18445 | CD1 | LEU | F | 52 | 58.463 | 10.212 | −31.562 | 1.00 | 56.42 | F |
| ATOM | 18446 | CD2 | LEU | F | 52 | 60.195 | 9.930 | −29.787 | 1.00 | 48.72 | F |
| ATOM | 18447 | C | LEU | F | 52 | 58.177 | 13.633 | −31.187 | 1.00 | 43.41 | F |
| ATOM | 18448 | O | LEU | F | 52 | 58.396 | 14.306 | −30.183 | 1.00 | 43.98 | F |
| ATOM | 18449 | N | ILE | F | 53 | 56.956 | 13.435 | −31.667 | 1.00 | 39.04 | F |
| ATOM | 18450 | CA | ILE | F | 53 | 55.773 | 13.968 | −30.998 | 1.00 | 35.64 | F |
| ATOM | 18451 | CB | ILE | F | 53 | 54.939 | 14.822 | −31.954 | 1.00 | 42.75 | F |
| ATOM | 18452 | CG2 | ILE | F | 53 | 53.760 | 15.417 | −31.225 | 1.00 | 43.39 | F |
| ATOM | 18453 | CG1 | ILE | F | 53 | 55.799 | 15.926 | −32.545 | 1.00 | 41.64 | F |
| ATOM | 18454 | CD1 | ILE | F | 53 | 55.051 | 16.764 | −33.540 | 1.00 | 37.46 | F |
| ATOM | 18455 | C | ILE | F | 53 | 54.911 | 12.809 | −30.521 | 1.00 | 35.70 | F |
| ATOM | 18456 | O | ILE | F | 53 | 54.755 | 11.815 | −31.230 | 1.00 | 38.78 | F |
| ATOM | 18457 | N | TYR | F | 54 | 54.346 | 12.931 | −29.329 | 1.00 | 38.30 | F |
| ATOM | 18458 | CA | TYR | F | 54 | 53.525 | 11.851 | −28.806 | 1.00 | 35.43 | F |
| ATOM | 18459 | CB | TYR | F | 54 | 54.426 | 10.761 | −28.207 | 1.00 | 34.13 | F |
| ATOM | 18460 | CG | TYR | F | 54 | 55.211 | 11.229 | −26.999 | 1.00 | 34.13 | F |
| ATOM | 18461 | CD1 | TYR | F | 54 | 54.658 | 11.155 | −25.715 | 1.00 | 34.13 | F |
| ATOM | 18462 | CE1 | TYR | F | 54 | 55.338 | 11.638 | −24.607 | 1.00 | 34.13 | F |
| ATOM | 18463 | CD2 | TYR | F | 54 | 56.475 | 11.798 | −27.141 | 1.00 | 34.13 | F |
| ATOM | 18464 | CE2 | TYR | F | 54 | 57.162 | 12.285 | −26.038 | 1.00 | 34.13 | F |
| ATOM | 18465 | CZ | TYR | F | 54 | 56.586 | 12.204 | −24.770 | 1.00 | 34.13 | F |
| ATOM | 18466 | OH | TYR | F | 54 | 57.237 | 12.688 | −23.656 | 1.00 | 34.13 | F |
| ATOM | 18467 | C | TYR | F | 54 | 52.523 | 12.339 | −27.756 | 1.00 | 34.02 | F |
| ATOM | 18468 | O | TYR | F | 54 | 52.673 | 13.425 | −27.174 | 1.00 | 32.87 | F |
| ATOM | 18469 | N | SER | F | 55 | 51.507 | 11.520 | −27.516 | 1.00 | 44.40 | F |
| ATOM | 18470 | CA | SER | F | 55 | 50.489 | 11.871 | −26.557 | 1.00 | 44.18 | F |
| ATOM | 18471 | CB | SER | F | 55 | 49.599 | 12.973 | −27.119 | 1.00 | 52.49 | F |
| ATOM | 18472 | OG | SER | F | 55 | 48.490 | 13.199 | −26.267 | 1.00 | 59.05 | F |
| ATOM | 18473 | C | SER | F | 55 | 49.624 | 10.685 | −26.210 | 1.00 | 47.16 | F |
| ATOM | 18474 | O | SER | F | 55 | 49.445 | 9.772 | −27.021 | 1.00 | 44.30 | F |
| ATOM | 18475 | N | GLN | F | 56 | 49.089 | 10.695 | −24.993 | 1.00 | 60.71 | F |
| ATOM | 18476 | CA | GLN | F | 56 | 48.204 | 9.630 | −24.570 | 1.00 | 61.41 | F |
| ATOM | 18477 | CB | GLN | F | 56 | 48.929 | 8.566 | −23.755 | 1.00 | 47.65 | F |
| ATOM | 18478 | CG | GLN | F | 56 | 47.992 | 7.411 | −23.444 | 1.00 | 49.25 | F |
| ATOM | 18479 | CD | GLN | F | 56 | 48.500 | 6.482 | −22.373 | 1.00 | 52.37 | F |
| ATOM | 18480 | OE1 | GLN | F | 56 | 48.410 | 6.784 | −21.187 | 1.00 | 51.90 | F |
| ATOM | 18481 | NE2 | GLN | F | 56 | 49.039 | 5.339 | −22.784 | 1.00 | 55.48 | F |
| ATOM | 18482 | C | GLN | F | 56 | 47.095 | 10.185 | −23.714 | 1.00 | 63.89 | F |
| ATOM | 18483 | O | GLN | F | 56 | 47.337 | 11.008 | −22.825 | 1.00 | 61.97 | F |
| ATOM | 18484 | N | VAL | F | 57 | 45.876 | 9.741 | −23.990 | 1.00 | 49.50 | F |
| ATOM | 18485 | CA | VAL | F | 57 | 44.737 | 10.160 | −23.193 | 1.00 | 43.87 | F |
| ATOM | 18486 | CB | VAL | F | 57 | 43.731 | 10.992 | −24.005 | 1.00 | 61.48 | F |
| ATOM | 18487 | CG1 | VAL | F | 57 | 44.415 | 12.223 | −24.562 | 1.00 | 62.68 | F |
| ATOM | 18488 | CG2 | VAL | F | 57 | 43.134 | 10.154 | −25.113 | 1.00 | 58.41 | F |
| ATOM | 18489 | C | VAL | F | 57 | 44.053 | 8.894 | −22.704 | 1.00 | 46.48 | F |
| ATOM | 18490 | O | VAL | F | 57 | 44.132 | 7.850 | −23.350 | 1.00 | 47.59 | F |
| ATOM | 18491 | N | LEU | F | 58 | 43.402 | 8.978 | −21.552 | 1.00 | 59.53 | F |
| ATOM | 18492 | CA | LEU | F | 58 | 42.701 | 7.825 | −21.012 | 1.00 | 57.98 | F |
| ATOM | 18493 | CB | LEU | F | 58 | 43.321 | 7.396 | −19.684 | 1.00 | 54.21 | F |
| ATOM | 18494 | CG | LEU | F | 58 | 43.176 | 5.925 | −19.293 | 1.00 | 56.23 | F |
| ATOM | 18495 | CD1 | LEU | F | 58 | 43.332 | 5.804 | −17.783 | 1.00 | 50.25 | F |
| ATOM | 18496 | CD2 | LEU | F | 58 | 41.828 | 5.390 | −19.722 | 1.00 | 54.01 | F |
| ATOM | 18497 | C | LEU | F | 58 | 41.251 | 8.225 | −20.791 | 1.00 | 60.95 | F |
| ATOM | 18498 | O | LEU | F | 58 | 40.961 | 9.110 | −19.980 | 1.00 | 62.58 | F |
| ATOM | 18499 | N | PHE | F | 59 | 40.343 | 7.589 | −21.523 | 1.00 | 68.11 | F |
| ATOM | 18500 | CA | PHE | F | 59 | 38.923 | 7.888 | −21.384 | 1.00 | 69.01 | F |
| ATOM | 18501 | CB | PHE | F | 59 | 38.209 | 7.791 | −22.728 | 1.00 | 53.79 | F |
| ATOM | 18502 | CG | PHE | F | 59 | 38.632 | 8.825 | −23.718 | 1.00 | 50.28 | F |
| ATOM | 18503 | CD1 | PHE | F | 59 | 39.490 | 8.500 | −24.756 | 1.00 | 48.72 | F |
| ATOM | 18504 | CD2 | PHE | F | 59 | 38.157 | 10.124 | −23.629 | 1.00 | 52.24 | F |
| ATOM | 18505 | CE1 | PHE | F | 59 | 39.868 | 9.453 | −25.697 | 1.00 | 54.64 | F |
| ATOM | 18506 | CE2 | PHE | F | 59 | 38.530 | 11.085 | −24.564 | 1.00 | 55.35 | F |
| ATOM | 18507 | CZ | PHE | F | 59 | 39.388 | 10.745 | −25.600 | 1.00 | 53.95 | F |
| ATOM | 18508 | C | PHE | F | 59 | 38.282 | 6.905 | −20.416 | 1.00 | 73.12 | F |
| ATOM | 18509 | O | PHE | F | 59 | 38.660 | 5.738 | −20.371 | 1.00 | 72.81 | F |
| ATOM | 18510 | N | LYS | F | 60 | 37.315 | 7.388 | −19.642 | 1.00 | 83.03 | F |
| ATOM | 18511 | CA | LYS | F | 60 | 36.610 | 6.558 | −18.678 | 1.00 | 84.38 | F |
| ATOM | 18512 | CB | LYS | F | 60 | 37.229 | 6.705 | −17.286 | 1.00 | 83.57 | F |
| ATOM | 18513 | CG | LYS | F | 60 | 36.492 | 5.940 | −16.198 | 1.00 | 89.76 | F |
| ATOM | 18514 | CD | LYS | F | 60 | 37.115 | 6.202 | −14.843 | 1.00 | 97.97 | F |
| ATOM | 18515 | CE | LYS | F | 60 | 36.374 | 5.476 | −13.738 | 1.00 | 100.72 | F |
| ATOM | 18516 | NZ | LYS | F | 60 | 37.037 | 5.701 | −12.424 | 1.00 | 101.06 | F |
| ATOM | 18517 | C | LYS | F | 60 | 35.154 | 6.980 | −18.630 | 1.00 | 83.89 | F |
| ATOM | 18518 | O | LYS | F | 60 | 34.840 | 8.170 | −18.640 | 1.00 | 86.64 | F |
| ATOM | 18519 | N | GLY | F | 61 | 34.266 | 5.999 | −18.581 | 1.00 | 90.96 | F |
| ATOM | 18520 | CA | GLY | F | 61 | 32.854 | 6.306 | −18.522 | 1.00 | 93.52 | F |
| ATOM | 18521 | C | GLY | F | 61 | 32.121 | 5.332 | −17.626 | 1.00 | 95.22 | F |
| ATOM | 18522 | O | GLY | F | 61 | 32.464 | 4.150 | −17.570 | 1.00 | 98.39 | F |
| ATOM | 18523 | N | GLN | F | 62 | 31.120 | 5.834 | −16.912 | 1.00 | 91.21 | F |
| ATOM | 18524 | CA | GLN | F | 62 | 30.314 | 5.001 | −16.030 | 1.00 | 91.52 | F |
| ATOM | 18525 | CB | GLN | F | 62 | 29.766 | 5.833 | −14.870 | 1.00 | 131.70 | F |
| ATOM | 18526 | CG | GLN | F | 62 | 30.833 | 6.526 | −14.039 | 1.00 | 135.83 | F |
| ATOM | 18527 | CD | GLN | F | 62 | 31.741 | 5.549 | −13.318 | 1.00 | 141.77 | F |
| ATOM | 18528 | OE1 | GLN | F | 62 | 32.412 | 4.730 | −13.944 | 1.00 | 141.37 | F |
| ATOM | 18529 | NE2 | GLN | F | 62 | 31.767 | 5.632 | −11.993 | 1.00 | 141.94 | F |
| ATOM | 18530 | C | GLN | F | 62 | 29.164 | 4.480 | −16.878 | 1.00 | 89.46 | F |
| ATOM | 18531 | O | GLN | F | 62 | 28.401 | 5.266 | −17.438 | 1.00 | 90.97 | F |
| ATOM | 18532 | N | GLY | F | 63 | 29.050 | 3.160 | −16.989 | 1.00 | 126.26 | F |
| ATOM | 18533 | CA | GLY | F | 63 | 27.982 | 2.583 | −17.788 | 1.00 | 124.25 | F |
| ATOM | 18534 | C | GLY | F | 63 | 27.980 | 3.147 | −19.197 | 1.00 | 122.33 | F |
| ATOM | 18535 | O | GLY | F | 63 | 28.927 | 3.826 | −19.593 | 1.00 | 120.37 | F |
| ATOM | 18536 | N | CYS | F | 64 | 26.916 | 2.892 | −19.954 | 1.00 | 144.97 | F |
| ATOM | 18537 | CA | CYS | F | 64 | 26.844 | 3.380 | −21.327 | 1.00 | 149.21 | F |
| ATOM | 18538 | C | CYS | F | 64 | 25.787 | 4.426 | −21.653 | 1.00 | 155.30 | F |
| ATOM | 18539 | O | CYS | F | 64 | 24.663 | 4.376 | −21.158 | 1.00 | 158.21 | F |
| ATOM | 18540 | CB | CYS | F | 64 | 26.693 | 2.204 | −22.286 | 1.00 | 147.95 | F |
| ATOM | 18541 | SG | CYS | F | 64 | 28.284 | 1.396 | −22.614 | 1.00 | 149.16 | F |
| ATOM | 18542 | N | PRO | F | 65 | 26.150 | 5.401 | −22.501 | 1.00 | 110.93 | F |
| ATOM | 18543 | CD | PRO | F | 65 | 27.535 | 5.716 | −22.887 | 1.00 | 148.74 | F |
| ATOM | 18544 | CA | PRO | F | 65 | 25.249 | 6.478 | −22.917 | 1.00 | 113.27 | F |
| ATOM | 18545 | CB | PRO | F | 65 | 26.192 | 7.486 | −23.577 | 1.00 | 151.27 | F |
| ATOM | 18546 | CG | PRO | F | 65 | 27.510 | 7.215 | −22.916 | 1.00 | 151.00 | F |
| ATOM | 18547 | C | PRO | F | 65 | 24.196 | 5.970 | −23.893 | 1.00 | 116.28 | F |
| ATOM | 18548 | O | PRO | F | 65 | 23.824 | 4.797 | −23.877 | 1.00 | 116.55 | F |
| ATOM | 18549 | N | SER | F | 66 | 23.732 | 6.868 | −24.752 | 1.00 | 162.81 | F |
| ATOM | 18550 | CA | SER | F | 66 | 22.729 | 6.532 | −25.747 | 1.00 | 166.41 | F |
| ATOM | 18551 | CB | SER | F | 66 | 21.742 | 7.689 | −25.899 | 1.00 | 156.51 | F |
| ATOM | 18552 | OG | SER | F | 66 | 20.777 | 7.411 | −26.897 | 1.00 | 153.67 | F |
| ATOM | 18553 | C | SER | F | 66 | 23.396 | 6.248 | −27.086 | 1.00 | 166.96 | F |
| ATOM | 18554 | O | SER | F | 66 | 24.615 | 6.376 | −27.224 | 1.00 | 160.38 | F |
| ATOM | 18555 | N | THR | F | 67 | 22.580 | 5.863 | −28.063 | 1.00 | 200.84 | F |
| ATOM | 18556 | CA | THR | F | 67 | 23.032 | 5.550 | −29.417 | 1.00 | 200.84 | F |
| ATOM | 18557 | CB | THR | F | 67 | 22.869 | 6.771 | −30.355 | 1.00 | 171.92 | F |
| ATOM | 18558 | OG1 | THR | F | 67 | 21.523 | 7.255 | −30.282 | 1.00 | 171.92 | F |
| ATOM | 18559 | CG2 | THR | F | 67 | 23.180 | 6.383 | −31.802 | 1.00 | 171.92 | F |
| ATOM | 18560 | C | THR | F | 67 | 24.480 | 5.069 | −29.502 | 1.00 | 200.84 | F |
| ATOM | 18561 | O | THR | F | 67 | 24.771 | 3.896 | −29.258 | 1.00 | 200.84 | F |
| ATOM | 18562 | N | HIS | F | 68 | 25.384 | 5.982 | −29.845 | 1.00 | 144.93 | F |
| ATOM | 18563 | CA | HIS | F | 68 | 26.790 | 5.641 | −29.986 | 1.00 | 140.05 | F |
| ATOM | 18564 | CB | HIS | F | 68 | 27.057 | 5.196 | −31.429 | 1.00 | 189.02 | F |
| ATOM | 18565 | CG | HIS | F | 68 | 28.279 | 4.347 | −31.589 | 1.00 | 189.02 | F |
| ATOM | 18566 | CD2 | HIS | F | 68 | 29.405 | 4.532 | −32.317 | 1.00 | 189.02 | F |
| ATOM | 18567 | ND1 | HIS | F | 68 | 28.424 | 3.127 | −30.963 | 1.00 | 189.02 | F |
| ATOM | 18568 | CE1 | HIS | F | 68 | 29.587 | 2.599 | −31.297 | 1.00 | 189.02 | F |
| ATOM | 18569 | NE2 | HIS | F | 68 | 30.202 | 3.431 | −32.118 | 1.00 | 189.02 | F |
| ATOM | 18570 | C | HIS | F | 68 | 27.665 | 6.842 | −29.638 | 1.00 | 135.88 | F |
| ATOM | 18571 | O | HIS | F | 68 | 27.314 | 7.982 | −29.940 | 1.00 | 135.33 | F |
| ATOM | 18572 | N | VAL | F | 69 | 28.800 | 6.577 | −28.997 | 1.00 | 94.93 | F |
| ATOM | 18573 | CA | VAL | F | 69 | 29.740 | 7.626 | −28.612 | 1.00 | 94.66 | F |
| ATOM | 18574 | CB | VAL | F | 69 | 29.928 | 7.685 | −27.080 | 1.00 | 109.60 | F |
| ATOM | 18575 | CG1 | VAL | F | 69 | 30.864 | 8.822 | −26.717 | 1.00 | 109.36 | F |
| ATOM | 18576 | CG2 | VAL | F | 69 | 28.585 | 7.869 | −26.396 | 1.00 | 107.86 | F |
| ATOM | 18577 | C | VAL | F | 69 | 31.101 | 7.374 | −29.252 | 1.00 | 94.25 | F |
| ATOM | 18578 | O | VAL | F | 69 | 31.615 | 6.254 | −29.226 | 1.00 | 97.81 | F |
| ATOM | 18579 | N | LEU | F | 70 | 31.675 | 8.422 | −29.833 | 1.00 | 82.01 | F |
| ATOM | 18580 | CA | LEU | F | 70 | 32.980 | 8.323 | −30.474 | 1.00 | 77.23 | F |
| ATOM | 18581 | CB | LEU | F | 70 | 32.925 | 8.827 | −31.918 | 1.00 | 81.76 | F |
| ATOM | 18582 | CG | LEU | F | 70 | 32.130 | 7.997 | −32.922 | 1.00 | 83.46 | F |
| ATOM | 18583 | CD1 | LEU | F | 70 | 32.290 | 8.618 | −34.306 | 1.00 | 78.50 | F |
| ATOM | 18584 | CD2 | LEU | F | 70 | 32.617 | 6.545 | −32.915 | 1.00 | 84.06 | F |
| ATOM | 18585 | C | LEU | F | 70 | 34.014 | 9.133 | −29.718 | 1.00 | 78.27 | F |
| ATOM | 18586 | O | LEU | F | 70 | 33.771 | 10.277 | −29.340 | 1.00 | 78.38 | F |
| ATOM | 18587 | N | LEU | F | 71 | 35.173 | 8.528 | −29.504 | 1.00 | 71.30 | F |
| ATOM | 18588 | CA | LEU | F | 71 | 36.267 | 9.181 | −28.809 | 1.00 | 71.24 | F |
| ATOM | 18589 | CB | LEU | F | 71 | 36.773 | 8.285 | −27.681 | 1.00 | 65.53 | F |
| ATOM | 18590 | CG | LEU | F | 71 | 35.746 | 7.909 | −26.607 | 1.00 | 63.20 | F |
| ATOM | 18591 | CD1 | LEU | F | 71 | 36.364 | 6.939 | −25.605 | 1.00 | 59.35 | F |
| ATOM | 18592 | CD2 | LEU | F | 71 | 35.264 | 9.172 | −25.912 | 1.00 | 63.32 | F |
| ATOM | 18593 | C | LEU | F | 71 | 37.387 | 9.418 | −29.808 | 1.00 | 75.27 | F |
| ATOM | 18594 | O | LEU | F | 71 | 37.827 | 8.487 | −30.484 | 1.00 | 74.76 | F |
| ATOM | 18595 | N | THR | F | 72 | 37.840 | 10.661 | −29.914 | 1.00 | 58.33 | F |
| ATOM | 18596 | CA | THR | F | 72 | 38.918 | 10.980 | −30.836 | 1.00 | 55.76 | F |
| ATOM | 18597 | CB | THR | F | 72 | 38.448 | 11.863 | −32.003 | 1.00 | 67.10 | F |
| ATOM | 18598 | OG1 | THR | F | 72 | 38.297 | 13.217 | −31.553 | 1.00 | 69.63 | F |
| ATOM | 18599 | CG2 | THR | F | 72 | 37.132 | 11.357 | −32.552 | 1.00 | 64.82 | F |
| ATOM | 18600 | C | THR | F | 72 | 40.048 | 11.713 | −30.131 | 1.00 | 58.39 | F |
| ATOM | 18601 | O | THR | F | 72 | 39.830 | 12.552 | −29.253 | 1.00 | 59.43 | F |
| ATOM | 18602 | N | HIS | F | 73 | 41.264 | 11.370 | −30.526 | 1.00 | 57.57 | F |
| ATOM | 18603 | CA | HIS | F | 73 | 42.458 | 11.984 | −29.988 | 1.00 | 58.02 | F |
| ATOM | 18604 | CB | HIS | F | 73 | 43.244 | 10.968 | −29.187 | 1.00 | 47.93 | F |
| ATOM | 18605 | CG | HIS | F | 73 | 44.429 | 11.550 | −28.499 | 1.00 | 50.80 | F |
| ATOM | 18606 | CD2 | HIS | F | 73 | 44.751 | 12.832 | −28.213 | 1.00 | 52.56 | F |
| ATOM | 18607 | ND1 | HIS | F | 73 | 45.457 | 10.774 | −28.010 | 1.00 | 55.41 | F |
| ATOM | 18608 | CE1 | HIS | F | 73 | 46.364 | 11.556 | −27.451 | 1.00 | 53.95 | F |
| ATOM | 18609 | NE2 | HIS | F | 73 | 45.959 | 12.808 | −27.561 | 1.00 | 54.12 | F |
| ATOM | 18610 | C | HIS | F | 73 | 43.249 | 12.390 | −31.216 | 1.00 | 57.10 | F |
| ATOM | 18611 | O | HIS | F | 73 | 43.384 | 11.602 | −32.154 | 1.00 | 52.96 | F |
| ATOM | 18612 | N | THR | F | 74 | 43.763 | 13.612 | −31.236 | 1.00 | 50.02 | F |
| ATOM | 18613 | CA | THR | F | 74 | 44.506 | 14.043 | −32.407 | 1.00 | 56.61 | F |
| ATOM | 18614 | CB | THR | F | 74 | 43.516 | 14.458 | −33.564 | 1.00 | 55.62 | F |
| ATOM | 18615 | OG1 | THR | F | 74 | 43.929 | 15.694 | −34.145 | 1.00 | 58.13 | F |
| ATOM | 18616 | CG2 | THR | F | 74 | 42.087 | 14.594 | −33.048 | 1.00 | 60.45 | F |
| ATOM | 18617 | C | THR | F | 74 | 45.516 | 15.155 | −32.136 | 1.00 | 55.46 | F |
| ATOM | 18618 | O | THR | F | 74 | 45.246 | 16.089 | −31.381 | 1.00 | 51.04 | F |
| ATOM | 18619 | N | ILE | F | 75 | 46.695 | 15.028 | −32.741 | 1.00 | 41.13 | F |
| ATOM | 18620 | CA | ILE | F | 75 | 47.744 | 16.029 | −32.592 | 1.00 | 42.43 | F |
| ATOM | 18621 | CB | ILE | F | 75 | 49.116 | 15.372 | −32.347 | 1.00 | 44.85 | F |
| ATOM | 18622 | CG2 | ILE | F | 75 | 50.208 | 16.430 | −32.308 | 1.00 | 44.34 | F |
| ATOM | 18623 | CG1 | ILE | F | 75 | 49.079 | 14.599 | −31.031 | 1.00 | 42.01 | F |
| ATOM | 18624 | CD1 | ILE | F | 75 | 50.401 | 13.965 | −30.660 | 1.00 | 45.30 | F |
| ATOM | 18625 | C | ILE | F | 75 | 47.793 | 16.843 | −33.878 | 1.00 | 45.57 | F |
| ATOM | 18626 | O | ILE | F | 75 | 47.958 | 16.294 | −34.976 | 1.00 | 42.34 | F |
| ATOM | 18627 | N | SER | F | 76 | 47.629 | 18.154 | −33.742 | 1.00 | 54.94 | F |
| ATOM | 18628 | CA | SER | F | 76 | 47.637 | 19.041 | −34.896 | 1.00 | 56.02 | F |
| ATOM | 18629 | CB | SER | F | 76 | 46.407 | 19.946 | −34.889 | 1.00 | 79.68 | F |
| ATOM | 18630 | OG | SER | F | 76 | 45.215 | 19.196 | −34.857 | 1.00 | 88.97 | F |
| ATOM | 18631 | C | SER | F | 76 | 48.855 | 19.932 | −34.922 | 1.00 | 52.02 | F |
| ATOM | 18632 | O | SER | F | 76 | 49.456 | 20.217 | −33.893 | 1.00 | 48.17 | F |
| ATOM | 18633 | N | ARG | F | 77 | 49.210 | 20.368 | −36.121 | 1.00 | 45.04 | F |
| ATOM | 18634 | CA | ARG | F | 77 | 50.307 | 21.286 | −36.295 | 1.00 | 50.22 | F |
| ATOM | 18635 | CB | ARG | F | 77 | 51.397 | 20.705 | −37.200 | 1.00 | 39.50 | F |
| ATOM | 18636 | CG | ARG | F | 77 | 52.278 | 21.807 | −37.846 | 1.00 | 41.68 | F |
| ATOM | 18637 | CD | ARG | F | 77 | 53.661 | 21.324 | −38.244 | 1.00 | 42.55 | F |
| ATOM | 18638 | NE | ARG | F | 77 | 53.838 | 21.178 | −39.683 | 1.00 | 44.99 | F |
| ATOM | 18639 | CZ | ARG | F | 77 | 54.800 | 21.781 | −40.376 | 1.00 | 48.42 | F |
| ATOM | 18640 | NH1 | ARG | F | 77 | 54.896 | 21.590 | −41.684 | 1.00 | 48.51 | F |
| ATOM | 18641 | NH2 | ARG | F | 77 | 55.657 | 22.586 | −39.768 | 1.00 | 48.28 | F |
| ATOM | 18642 | C | ARG | F | 77 | 49.731 | 22.517 | −36.968 | 1.00 | 48.56 | F |
| ATOM | 18643 | O | ARG | F | 77 | 48.943 | 22.410 | −37.912 | 1.00 | 54.31 | F |
| ATOM | 18644 | N | ILE | F | 78 | 50.082 | 23.683 | −36.455 | 1.00 | 41.34 | F |
| ATOM | 18645 | CA | ILE | F | 78 | 49.658 | 24.914 | −37.092 | 1.00 | 40.03 | F |
| ATOM | 18646 | CB | ILE | F | 78 | 48.699 | 25.792 | −36.166 | 1.00 | 61.56 | F |
| ATOM | 18647 | CG2 | ILE | F | 78 | 48.210 | 24.990 | −34.984 | 1.00 | 62.06 | F |
| ATOM | 18648 | CG1 | ILE | F | 78 | 49.387 | 27.058 | −35.697 | 1.00 | 63.23 | F |
| ATOM | 18649 | CD1 | ILE | F | 78 | 49.560 | 28.086 | −36.790 | 1.00 | 63.55 | F |
| ATOM | 18650 | C | ILE | F | 78 | 51.030 | 25.540 | −37.398 | 1.00 | 42.20 | F |
| ATOM | 18651 | O | ILE | F | 78 | 51.719 | 26.074 | −36.532 | 1.00 | 40.75 | F |
| ATOM | 18652 | N | ALA | F | 79 | 51.446 | 25.405 | −38.645 | 1.00 | 49.54 | F |
| ATOM | 18653 | CA | ALA | F | 79 | 52.742 | 25.900 | −39.079 | 1.00 | 46.14 | F |
| ATOM | 18654 | CB | ALA | F | 79 | 53.095 | 25.276 | −40.409 | 1.00 | 58.07 | F |
| ATOM | 18655 | C | ALA | F | 79 | 52.858 | 27.410 | −39.182 | 1.00 | 47.34 | F |
| ATOM | 18656 | O | ALA | F | 79 | 51.883 | 28.105 | −39.446 | 1.00 | 47.54 | F |
| ATOM | 18657 | N | VAL | F | 80 | 54.069 | 27.912 | −38.989 | 1.00 | 52.87 | F |
| ATOM | 18658 | CA | VAL | F | 80 | 54.312 | 29.344 | −39.073 | 1.00 | 55.36 | F |
| ATOM | 18659 | CB | VAL | F | 80 | 55.764 | 29.703 | −38.707 | 1.00 | 57.63 | F |
| ATOM | 18660 | CG1 | VAL | F | 80 | 55.985 | 31.196 | −38.865 | 1.00 | 56.03 | F |
| ATOM | 18661 | CG2 | VAL | F | 80 | 56.059 | 29.277 | −37.289 | 1.00 | 59.33 | F |
| ATOM | 18662 | C | VAL | F | 80 | 54.058 | 29.813 | −40.492 | 1.00 | 59.91 | F |
| ATOM | 18663 | O | VAL | F | 80 | 53.621 | 30.938 | −40.709 | 1.00 | 58.50 | F |
| ATOM | 18664 | N | SER | F | 81 | 54.332 | 28.945 | −41.459 | 1.00 | 69.48 | F |
| ATOM | 18665 | CA | SER | F | 81 | 54.127 | 29.297 | −42.856 | 1.00 | 72.34 | F |
| ATOM | 18666 | CB | SER | F | 81 | 55.220 | 28.675 | −43.709 | 1.00 | 44.56 | F |
| ATOM | 18667 | OG | SER | F | 81 | 56.481 | 29.195 | −43.342 | 1.00 | 47.60 | F |
| ATOM | 18668 | C | SER | F | 81 | 52.760 | 28.904 | −43.414 | 1.00 | 76.52 | F |
| ATOM | 18669 | O | SER | F | 81 | 52.541 | 29.006 | −44.618 | 1.00 | 77.04 | F |
| ATOM | 18670 | N | TYR | F | 82 | 51.851 | 28.458 | −42.547 | 1.00 | 51.40 | F |
| ATOM | 18671 | CA | TYR | F | 82 | 50.512 | 28.069 | −42.965 | 1.00 | 53.44 | F |
| ATOM | 18672 | CB | TYR | F | 82 | 50.565 | 26.755 | −43.758 | 1.00 | 37.26 | F |
| ATOM | 18673 | CG | TYR | F | 82 | 49.261 | 26.393 | −44.447 | 1.00 | 40.46 | F |
| ATOM | 18674 | CD1 | TYR | F | 82 | 48.589 | 25.201 | −44.150 | 1.00 | 42.75 | F |
| ATOM | 18675 | CE1 | TYR | F | 82 | 47.355 | 24.900 | −44.732 | 1.00 | 44.54 | F |
| ATOM | 18676 | CD2 | TYR | F | 82 | 48.666 | 27.266 | −45.351 | 1.00 | 42.55 | F |
| ATOM | 18677 | CE2 | TYR | F | 82 | 47.435 | 26.975 | −45.935 | 1.00 | 44.02 | F |
| ATOM | 18678 | CZ | TYR | F | 82 | 46.788 | 25.799 | −45.621 | 1.00 | 44.71 | F |
| ATOM | 18679 | OH | TYR | F | 82 | 45.567 | 25.551 | −46.196 | 1.00 | 43.13 | F |
| ATOM | 18680 | C | TYR | F | 82 | 49.628 | 27.914 | −41.730 | 1.00 | 52.87 | F |
| ATOM | 18681 | O | TYR | F | 82 | 49.309 | 26.791 | −41.324 | 1.00 | 53.05 | F |
| ATOM | 18682 | N | GLN | F | 83 | 49.237 | 29.051 | −41.146 | 1.00 | 58.56 | F |
| ATOM | 18683 | CA | GLN | F | 83 | 48.405 | 29.097 | −39.941 | 1.00 | 58.45 | F |
| ATOM | 18684 | CB | GLN | F | 83 | 48.085 | 30.540 | −39.569 | 1.00 | 63.37 | F |
| ATOM | 18685 | CG | GLN | F | 83 | 48.960 | 31.155 | −38.491 | 1.00 | 67.64 | F |
| ATOM | 18686 | CD | GLN | F | 83 | 50.322 | 31.547 | −39.001 | 1.00 | 68.68 | F |
| ATOM | 18687 | OE1 | GLN | F | 83 | 51.055 | 32.280 | −38.342 | 1.00 | 67.70 | F |
| ATOM | 18688 | NE2 | GLN | F | 83 | 50.672 | 31.061 | −40.184 | 1.00 | 66.18 | F |
| ATOM | 18689 | C | GLN | F | 83 | 47.083 | 28.358 | −39.985 | 1.00 | 56.63 | F |
| ATOM | 18690 | O | GLN | F | 83 | 46.041 | 28.998 | −40.027 | 1.00 | 54.08 | F |
| ATOM | 18691 | N | THR | F | 84 | 47.102 | 27.030 | −39.959 | 1.00 | 64.96 | F |
| ATOM | 18692 | CA | THR | F | 84 | 45.859 | 26.264 | −39.968 | 1.00 | 57.33 | F |
| ATOM | 18693 | CB | THR | F | 84 | 45.321 | 26.018 | −41.379 | 1.00 | 72.84 | F |
| ATOM | 18694 | OG1 | THR | F | 84 | 45.275 | 27.250 | −42.099 | 1.00 | 73.39 | F |
| ATOM | 18695 | CG2 | THR | F | 84 | 43.921 | 25.423 | −41.311 | 1.00 | 75.59 | F |
| ATOM | 18696 | C | THR | F | 84 | 46.130 | 24.907 | −39.373 | 1.00 | 53.84 | F |
| ATOM | 18697 | O | THR | F | 84 | 47.148 | 24.284 | −39.674 | 1.00 | 55.15 | F |
| ATOM | 18698 | N | LYS | F | 85 | 45.221 | 24.442 | −38.530 | 1.00 | 43.22 | F |
| ATOM | 18699 | CA | LYS | F | 85 | 45.390 | 23.144 | −37.937 | 1.00 | 43.08 | F |
| ATOM | 18700 | CB | LYS | F | 85 | 44.248 | 22.842 | −36.969 | 1.00 | 70.90 | F |
| ATOM | 18701 | CG | LYS | F | 85 | 44.493 | 23.218 | −35.522 | 1.00 | 67.84 | F |
| ATOM | 18702 | CD | LYS | F | 85 | 43.368 | 22.647 | −34.665 | 1.00 | 66.24 | F |
| ATOM | 18703 | CE | LYS | F | 85 | 43.607 | 22.857 | −33.179 | 1.00 | 64.54 | F |
| ATOM | 18704 | NZ | LYS | F | 85 | 43.695 | 24.300 | −32.797 | 1.00 | 62.79 | F |
| ATOM | 18705 | C | LYS | F | 85 | 45.384 | 22.090 | −39.039 | 1.00 | 42.88 | F |
| ATOM | 18706 | O | LYS | F | 85 | 44.462 | 22.037 | −39.851 | 1.00 | 43.33 | F |
| ATOM | 18707 | N | VAL | F | 86 | 46.439 | 21.282 | −39.099 | 1.00 | 43.01 | F |
| ATOM | 18708 | CA | VAL | F | 86 | 46.492 | 20.175 | −40.041 | 1.00 | 45.90 | F |
| ATOM | 18709 | CB | VAL | F | 86 | 47.557 | 20.366 | −41.170 | 1.00 | 43.22 | F |
| ATOM | 18710 | CG1 | VAL | F | 86 | 47.197 | 21.555 | −42.020 | 1.00 | 49.96 | F |
| ATOM | 18711 | CG2 | VAL | F | 86 | 48.932 | 20.555 | −40.586 | 1.00 | 44.49 | F |
| ATOM | 18712 | C | VAL | F | 86 | 46.844 | 18.995 | −39.141 | 1.00 | 46.57 | F |
| ATOM | 18713 | O | VAL | F | 86 | 47.770 | 19.077 | −38.344 | 1.00 | 51.26 | F |
| ATOM | 18714 | N | ASN | F | 87 | 46.080 | 17.916 | −39.229 | 1.00 | 58.46 | F |
| ATOM | 18715 | CA | ASN | F | 87 | 46.332 | 16.754 | −38.394 | 1.00 | 55.14 | F |
| ATOM | 18716 | CB | ASN | F | 87 | 45.138 | 15.818 | −38.458 | 1.00 | 58.92 | F |
| ATOM | 18717 | CG | ASN | F | 87 | 43.904 | 16.427 | −37.850 | 1.00 | 59.22 | F |
| ATOM | 18718 | OD1 | ASN | F | 87 | 43.964 | 17.493 | −37.240 | 1.00 | 61.83 | F |
| ATOM | 18719 | ND2 | ASN | F | 87 | 42.771 | 15.746 | −38.000 | 1.00 | 56.18 | F |
| ATOM | 18720 | C | ASN | F | 87 | 47.590 | 15.968 | −38.734 | 1.00 | 53.96 | F |
| ATOM | 18721 | O | ASN | F | 87 | 47.824 | 15.627 | −39.893 | 1.00 | 52.54 | F |
| ATOM | 18722 | N | LEU | F | 88 | 48.392 | 15.667 | −37.717 | 1.00 | 50.27 | F |
| ATOM | 18723 | CA | LEU | F | 88 | 49.610 | 14.897 | −37.923 | 1.00 | 49.26 | F |
| ATOM | 18724 | CB | LEU | F | 88 | 50.759 | 15.450 | −37.089 | 1.00 | 30.57 | F |
| ATOM | 18725 | CG | LEU | F | 88 | 51.181 | 16.882 | −37.362 | 1.00 | 27.50 | F |
| ATOM | 18726 | CD1 | LEU | F | 88 | 52.353 | 17.217 | −36.448 | 1.00 | 28.27 | F |
| ATOM | 18727 | CD2 | LEU | F | 88 | 51.537 | 17.049 | −38.820 | 1.00 | 26.07 | F |
| ATOM | 18728 | C | LEU | F | 88 | 49.363 | 13.468 | −37.502 | 1.00 | 48.50 | F |
| ATOM | 18729 | O | LEU | F | 88 | 49.763 | 12.533 | −38.178 | 1.00 | 46.97 | F |
| ATOM | 18730 | N | LEU | F | 89 | 48.713 | 13.313 | −36.360 | 1.00 | 40.13 | F |
| ATOM | 18731 | CA | LEU | F | 89 | 48.411 | 11.999 | −35.811 | 1.00 | 39.05 | F |
| ATOM | 18732 | CB | LEU | F | 89 | 49.371 | 11.678 | −34.675 | 1.00 | 40.57 | F |
| ATOM | 18733 | CG | LEU | F | 89 | 50.851 | 11.806 | −34.980 | 1.00 | 44.55 | F |
| ATOM | 18734 | CD1 | LEU | F | 89 | 51.600 | 11.933 | −33.675 | 1.00 | 44.28 | F |
| ATOM | 18735 | CD2 | LEU | F | 89 | 51.324 | 10.612 | −35.762 | 1.00 | 43.67 | F |
| ATOM | 18736 | C | LEU | F | 89 | 46.988 | 12.037 | −35.272 | 1.00 | 40.72 | F |
| ATOM | 18737 | O | LEU | F | 89 | 46.573 | 13.023 | −34.662 | 1.00 | 41.44 | F |
| ATOM | 18738 | N | SER | F | 90 | 46.243 | 10.965 | −35.494 | 1.00 | 42.81 | F |
| ATOM | 18739 | CA | SER | F | 90 | 44.864 | 10.914 | −35.044 | 1.00 | 42.15 | F |
| ATOM | 18740 | CB | SER | F | 90 | 43.983 | 11.721 | −35.992 | 1.00 | 67.96 | F |
| ATOM | 18741 | OG | SER | F | 90 | 42.627 | 11.631 | −35.604 | 1.00 | 72.18 | F |
| ATOM | 18742 | C | SER | F | 90 | 44.320 | 9.495 | −34.948 | 1.00 | 40.78 | F |
| ATOM | 18743 | O | SER | F | 90 | 44.664 | 8.618 | −35.748 | 1.00 | 40.60 | F |
| ATOM | 18744 | N | ALA | F | 91 | 43.469 | 9.261 | −33.960 | 1.00 | 46.48 | F |
| ATOM | 18745 | CA | ALA | F | 91 | 42.879 | 7.940 | −33.802 | 1.00 | 44.57 | F |
| ATOM | 18746 | CB | ALA | F | 91 | 43.720 | 7.085 | −32.854 | 1.00 | 39.45 | F |
| ATOM | 18747 | C | ALA | F | 91 | 41.457 | 8.086 | −33.286 | 1.00 | 48.54 | F |
| ATOM | 18740 | O | ALA | F | 91 | 41.099 | 9.118 | −32.719 | 1.00 | 50.41 | F |
| ATOM | 18749 | N | ILE | F | 92 | 40.647 | 7.055 | −33.499 | 1.00 | 66.29 | F |
| ATOM | 18750 | CA | ILE | F | 92 | 39.255 | 7.064 | −33.066 | 1.00 | 66.27 | F |
| ATOM | 18751 | CB | ILE | F | 92 | 38.309 | 7.154 | −34.267 | 1.00 | 59.03 | F |
| ATOM | 18752 | CG2 | ILE | F | 92 | 36.883 | 7.279 | −33.792 | 1.00 | 65.10 | F |
| ATOM | 18753 | CG1 | ILE | F | 92 | 38.681 | 8.364 | −35.127 | 1.00 | 57.14 | F |
| ATOM | 18754 | CD1 | ILE | F | 92 | 37.921 | 8.448 | −36.433 | 1.00 | 63.01 | F |
| ATOM | 18755 | C | ILE | F | 92 | 38.976 | 5.772 | −32.330 | 1.00 | 66.10 | F |
| ATOM | 18756 | O | ILE | F | 92 | 39.555 | 4.743 | −32.661 | 1.00 | 67.18 | F |
| ATOM | 18757 | N | LYS | F | 93 | 38.100 | 5.821 | −31.332 | 1.00 | 83.59 | F |
| ATOM | 18758 | CA | LYS | F | 93 | 37.773 | 4.624 | −30.569 | 1.00 | 84.24 | F |
| ATOM | 18759 | CB | LYS | F | 93 | 38.672 | 4.539 | −29.331 | 1.00 | 60.33 | F |
| ATOM | 18760 | CG | LYS | F | 93 | 38.977 | 3.120 | −28.889 | 1.00 | 64.02 | F |
| ATOM | 18761 | CD | LYS | F | 93 | 40.281 | 3.055 | −28.099 | 1.00 | 63.79 | F |
| ATOM | 18762 | CE | LYS | F | 93 | 40.659 | 1.609 | −27.766 | 1.00 | 63.69 | F |
| ATOM | 18763 | NZ | LYS | F | 93 | 41.959 | 1.482 | −27.028 | 1.00 | 66.26 | F |
| ATOM | 18764 | C | LYS | F | 93 | 36.303 | 4.657 | −30.169 | 1.00 | 85.94 | F |
| ATOM | 18765 | O | LYS | F | 93 | 35.804 | 5.697 | −29.740 | 1.00 | 83.86 | F |
| ATOM | 18766 | N | SER | F | 94 | 35.616 | 3.525 | −30.326 | 1.00 | 89.46 | F |
| ATOM | 18767 | CA | SER | F | 94 | 34.191 | 3.409 | −29.996 | 1.00 | 91.91 | F |
| ATOM | 18768 | CB | SER | F | 94 | 33.430 | 2.753 | −31.151 | 1.00 | 112.58 | F |
| ATOM | 18769 | OG | SER | F | 94 | 33.949 | 1.464 | −31.441 | 1.00 | 115.01 | F |
| ATOM | 18770 | C | SER | F | 94 | 34.007 | 2.573 | −28.737 | 1.00 | 94.50 | F |
| ATOM | 18771 | O | SER | F | 94 | 33.975 | 1.347 | −28.794 | 1.00 | 95.77 | F |
| ATOM | 18772 | N | PRO | F | 95 | 33.857 | 3.231 | −27.583 | 1.00 | 91.51 | F |
| ATOM | 18773 | CD | PRO | F | 95 | 33.626 | 4.682 | −27.443 | 1.00 | 86.69 | F |
| ATOM | 18774 | CA | PRO | F | 95 | 33.682 | 2.548 | −26.299 | 1.00 | 95.45 | F |
| ATOM | 18775 | CB | PRO | F | 95 | 33.703 | 3.698 | −25.300 | 1.00 | 89.63 | F |
| ATOM | 18776 | CG | PRO | F | 95 | 33.011 | 4.791 | −26.064 | 1.00 | 87.08 | F |
| ATOM | 18777 | C | PRO | F | 95 | 32.392 | 1.746 | −26.233 | 1.00 | 99.88 | F |
| ATOM | 18778 | O | PRO | F | 95 | 32.398 | 0.520 | −26.343 | 1.00 | 103.31 | F |
| ATOM | 18779 | N | CYS | F | 96 | 31.283 | 2.451 | −26.049 | 1.00 | 164.02 | F |
| ATOM | 18780 | CA | CYS | F | 96 | 29.987 | 1.809 | −25.968 | 1.00 | 163.81 | F |
| ATOM | 18781 | C | CYS | F | 96 | 29.480 | 1.481 | −27.355 | 1.00 | 164.91 | F |
| ATOM | 18782 | O | CYS | F | 96 | 29.100 | 2.368 | −28.117 | 1.00 | 165.21 | F |
| ATOM | 18783 | CB | CYS | F | 96 | 28.995 | 2.715 | −25.244 | 1.00 | 200.84 | F |
| ATOM | 18784 | SG | CYS | F | 96 | 29.357 | 2.893 | −23.470 | 1.00 | 200.84 | F |
| ATOM | 18785 | N | GLN | F | 97 | 29.495 | 0.194 | −27.676 | 1.00 | 200.78 | F |
| ATOM | 18786 | CA | GLN | F | 97 | 29.033 | −0.289 | −28.967 | 1.00 | 200.78 | F |
| ATOM | 18787 | CB | GLN | F | 97 | 29.584 | −1.696 | −29.212 | 1.00 | 159.03 | F |
| ATOM | 18788 | CG | GLN | F | 97 | 31.091 | −1.806 | −29.037 | 1.00 | 159.03 | F |
| ATOM | 18789 | CD | GLN | F | 97 | 31.558 | −3.241 | −28.870 | 1.00 | 159.03 | F |
| ATOM | 18790 | OE1 | GLN | F | 97 | 31.333 | −4.086 | −29.736 | 1.00 | 159.03 | F |
| ATOM | 18791 | NE2 | GLN | F | 97 | 32.214 | −3.522 | −27.750 | 1.00 | 159.03 | F |
| ATOM | 18792 | C | GLN | F | 97 | 27.505 | −0.317 | −28.956 | 1.00 | 200.78 | F |
| ATOM | 18793 | O | GLN | F | 97 | 26.891 | −1.283 | −29.408 | 1.00 | 200.78 | F |
| ATOM | 18794 | N | ARG | F | 98 | 26.906 | 0.749 | −28.426 | 1.00 | 200.84 | F |
| ATOM | 18795 | CA | ARG | F | 98 | 25.451 | 0.882 | −28.333 | 1.00 | 200.84 | F |
| ATOM | 18796 | CB | ARG | F | 98 | 24.808 | 0.573 | −29.695 | 1.00 | 200.84 | F |
| ATOM | 18797 | CG | ARG | F | 98 | 23.285 | 0.513 | −29.693 | 1.00 | 200.84 | F |
| ATOM | 18798 | CD | ARG | F | 98 | 22.647 | 1.847 | −29.332 | 1.00 | 200.84 | F |
| ATOM | 18799 | NE | ARG | F | 98 | 21.206 | 1.718 | −29.131 | 1.00 | 200.84 | F |
| ATOM | 18800 | CZ | ARG | F | 98 | 20.360 | 1.231 | −30.034 | 1.00 | 200.84 | F |
| ATOM | 18801 | NH1 | ARG | F | 98 | 19.067 | 1.153 | −29.754 | 1.00 | 200.84 | F |
| ATOM | 18802 | NH2 | ARG | F | 98 | 20.802 | 0.823 | −31.217 | 1.00 | 200.84 | F |
| ATOM | 18803 | C | ARG | F | 98 | 24.848 | −0.012 | −27.239 | 1.00 | 200.84 | F |
| ATOM | 18804 | O | ARG | F | 98 | 23.630 | −0.039 | −27.047 | 1.00 | 200.84 | F |
| ATOM | 18805 | N | GLU | F | 99 | 25.707 | −0.729 | −26.515 | 1.00 | 196.51 | F |
| ATOM | 18806 | CA | GLU | F | 99 | 25.264 | −1.619 | −25.440 | 1.00 | 196.51 | F |
| ATOM | 18807 | CB | GLU | F | 99 | 26.439 | −1.993 | −24.525 | 1.00 | 190.87 | F |
| ATOM | 18808 | CG | GLU | F | 99 | 27.736 | −1.242 | −24.805 | 1.00 | 190.87 | F |
| ATOM | 18809 | CD | GLU | F | 99 | 28.568 | −1.891 | −25.897 | 1.00 | 190.87 | F |
| ATOM | 18810 | OE1 | GLU | F | 99 | 27.988 | −2.286 | −26.929 | 1.00 | 190.87 | F |
| ATOM | 18811 | OE2 | GLU | F | 99 | 29.802 | −1.998 | −25.725 | 1.00 | 190.87 | F |
| ATOM | 18812 | C | GLU | F | 99 | 24.154 | −0.997 | −24.597 | 1.00 | 196.51 | F |
| ATOM | 18813 | O | GLU | F | 99 | 23.119 | −1.621 | −24.376 | 1.00 | 196.51 | F |
| ATOM | 18814 | N | THR | F | 100 | 24.382 | 0.231 | −24.135 | 1.00 | 198.08 | F |
| ATOM | 18815 | CA | THR | F | 100 | 23.421 | 0.963 | −23.311 | 1.00 | 198.08 | F |
| ATOM | 18816 | CB | THR | F | 100 | 22.234 | 1.491 | −24.157 | 1.00 | 154.69 | F |
| ATOM | 18817 | OG1 | THR | F | 100 | 21.614 | 0.404 | −24.854 | 1.00 | 154.97 | F |
| ATOM | 18818 | CG2 | THR | F | 100 | 22.712 | 2.525 | −25.166 | 1.00 | 155.86 | F |
| ATOM | 18819 | C | THR | F | 100 | 22.873 | 0.116 | −22.163 | 1.00 | 198.08 | F |
| ATOM | 18820 | O | THR | F | 100 | 22.186 | −0.880 | −22.383 | 1.00 | 198.08 | F |
| ATOM | 18821 | N | PRO | F | 101 | 23.170 | 0.511 | −20.914 | 1.00 | 200.84 | F |
| ATOM | 18822 | CD | PRO | F | 101 | 23.904 | 1.732 | −20.537 | 1.00 | 117.31 | F |
| ATOM | 18823 | CA | PRO | F | 101 | 22.715 | −0.204 | −19.719 | 1.00 | 200.84 | F |
| ATOM | 18824 | CB | PRO | F | 101 | 22.947 | 0.816 | −18.609 | 1.00 | 118.26 | F |
| ATOM | 18825 | CG | PRO | F | 101 | 24.193 | 1.501 | −19.067 | 1.00 | 116.29 | F |
| ATOM | 18826 | C | PRO | F | 101 | 21.260 | −0.668 | −19.799 | 1.00 | 200.84 | F |
| ATOM | 18827 | O | PRO | F | 101 | 20.334 | 0.144 | −19.788 | 1.00 | 200.84 | F |
| ATOM | 18828 | N | GLU | F | 102 | 21.076 | −1.983 | −19.880 | 1.00 | 191.64 | F |
| ATOM | 18829 | CA | GLU | F | 102 | 19.753 | −2.593 | −19.962 | 1.00 | 191.64 | F |
| ATOM | 18830 | CB | GLU | F | 102 | 19.881 | −4.109 | −19.750 | 1.00 | 154.58 | F |
| ATOM | 18831 | CG | GLU | F | 102 | 18.567 | −4.882 | −19.724 | 1.00 | 155.78 | F |
| ATOM | 18832 | CD | GLU | F | 102 | 17.781 | −4.668 | −18.442 | 1.00 | 157.09 | F |
| ATOM | 18833 | OE1 | GLU | F | 102 | 18.316 | −4.973 | −17.353 | 1.00 | 157.90 | F |
| ATOM | 18834 | OE2 | GLU | F | 102 | 16.628 | −4.193 | −18.526 | 1.00 | 156.21 | F |
| ATOM | 18835 | C | GLU | F | 102 | 18.780 | −1.993 | −18.946 | 1.00 | 188.20 | F |
| ATOM | 18836 | O | GLU | F | 102 | 17.606 | −1.780 | −19.250 | 1.00 | 185.26 | F |
| ATOM | 18837 | N | GLY | F | 103 | 19.275 | −1.718 | −17.743 | 1.00 | 145.71 | F |
| ATOM | 18838 | CA | GLY | F | 103 | 18.435 | −1.150 | −16.704 | 1.00 | 143.03 | F |
| ATOM | 18839 | C | GLY | F | 103 | 18.888 | −1.584 | −15.323 | 1.00 | 140.90 | F |
| ATOM | 18840 | O | GLY | F | 103 | 18.440 | −1.045 | −14.311 | 1.00 | 135.39 | F |
| ATOM | 18841 | N | ALA | F | 104 | 19.784 | −2.565 | −15.286 | 1.00 | 174.34 | F |
| ATOM | 18842 | CA | ALA | F | 104 | 20.319 | −3.083 | −14.033 | 1.00 | 174.34 | F |
| ATOM | 18843 | CB | ALA | F | 104 | 19.629 | −4.400 | −13.677 | 1.00 | 88.02 | F |
| ATOM | 18844 | C | ALA | F | 104 | 21.829 | −3.291 | −14.156 | 1.00 | 174.34 | F |
| ATOM | 18845 | O | ALA | F | 104 | 22.280 | −4.116 | −14.952 | 1.00 | 174.34 | F |
| ATOM | 18846 | N | GLU | F | 105 | 22.591 | −2.527 | −13.369 | 1.00 | 170.66 | F |
| ATOM | 18847 | CA | GLU | F | 105 | 24.059 | −2.575 | −13.332 | 1.00 | 170.66 | F |
| ATOM | 18848 | CB | GLU | F | 105 | 24.584 | −3.939 | −13.804 | 1.00 | 200.84 | F |
| ATOM | 18849 | CG | GLU | F | 105 | 24.255 | −5.095 | −12.875 | 1.00 | 200.84 | F |
| ATOM | 18850 | CD | GLU | F | 105 | 24.844 | −4.914 | −11.489 | 1.00 | 200.84 | F |
| ATOM | 18851 | OE1 | GLU | F | 105 | 24.442 | −3.960 | −10.789 | 1.00 | 200.84 | F |
| ATOM | 18852 | OE2 | GLU | F | 105 | 25.712 | −5.723 | −11.101 | 1.00 | 200.84 | F |
| ATOM | 18853 | C | GLU | F | 105 | 24.747 | −1.466 | −14.132 | 1.00 | 170.66 | F |
| ATOM | 18854 | O | GLU | F | 105 | 24.543 | −0.281 | −13.863 | 1.00 | 170.66 | F |
| ATOM | 18855 | N | ALA | F | 106 | 25.570 | −1.870 | −15.101 | 1.00 | 175.55 | F |
| ATOM | 18856 | CA | ALA | F | 106 | 26.328 | −0.963 | −15.970 | 1.00 | 175.55 | F |
| ATOM | 18857 | CB | ALA | F | 106 | 25.518 | 0.299 | −16.275 | 1.00 | 90.12 | F |
| ATOM | 18858 | C | ALA | F | 106 | 27.677 | −0.584 | −15.362 | 1.00 | 175.55 | F |
| ATOM | 18859 | O | ALA | F | 106 | 27.821 | 0.482 | −14.764 | 1.00 | 175.55 | F |
| ATOM | 18860 | N | LYS | F | 107 | 28.662 | −1.466 | −15.526 | 1.00 | 151.21 | F |
| ATOM | 18861 | CA | LYS | F | 107 | 30.011 | −1.245 | −15.005 | 1.00 | 145.33 | F |
| ATOM | 18862 | CB | LYS | F | 107 | 30.852 | −2.522 | −15.143 | 1.00 | 174.49 | F |
| ATOM | 18863 | CG | LYS | F | 107 | 30.548 | −3.627 | −14.138 | 1.00 | 174.44 | F |
| ATOM | 18864 | CD | LYS | F | 107 | 29.149 | −4.197 | −14.299 | 1.00 | 175.87 | F |
| ATOM | 18865 | CE | LYS | F | 107 | 28.959 | −5.426 | −13.419 | 1.00 | 171.91 | F |
| ATOM | 18866 | NZ | LYS | F | 107 | 29.277 | −5.150 | −11.989 | 1.00 | 172.17 | F |
| ATOM | 18867 | C | LYS | F | 107 | 30.720 | −0.106 | −15.739 | 1.00 | 140.81 | F |
| ATOM | 18868 | O | LYS | F | 107 | 30.244 | 0.370 | −16.772 | 1.00 | 139.94 | F |
| ATOM | 18869 | N | PRO | F | 108 | 31.869 | 0.353 | −15.205 | 1.00 | 115.17 | F |
| ATOM | 18870 | CD | PRO | F | 108 | 32.443 | 0.021 | −13.885 | 1.00 | 67.86 | F |
| ATOM | 18871 | CA | PRO | F | 108 | 32.628 | 1.439 | −15.836 | 1.00 | 113.91 | F |
| ATOM | 18872 | CB | PRO | F | 108 | 33.431 | 2.013 | −14.672 | 1.00 | 67.01 | F |
| ATOM | 18873 | CG | PRO | F | 108 | 33.762 | 0.792 | −13.887 | 1.00 | 64.39 | F |
| ATOM | 18874 | C | PRO | F | 108 | 33.530 | 0.892 | −16.943 | 1.00 | 113.14 | F |
| ATOM | 18875 | O | PRO | F | 108 | 33.851 | −0.298 | −16.960 | 1.00 | 111.32 | F |
| ATOM | 18876 | N | TRP | F | 109 | 33.926 | 1.760 | −17.870 | 1.00 | 95.77 | F |
| ATOM | 18877 | CA | TRP | F | 109 | 34.797 | 1.353 | −18.967 | 1.00 | 93.22 | F |
| ATOM | 18878 | CB | TRP | F | 109 | 34.033 | 1.297 | −20.291 | 1.00 | 78.76 | F |
| ATOM | 18879 | CG | TRP | F | 109 | 33.317 | 2.557 | −20.652 | 1.00 | 78.45 | F |
| ATOM | 18880 | CD2 | TRP | F | 109 | 33.873 | 3.709 | −21.296 | 1.00 | 77.46 | F |
| ATOM | 18881 | CE2 | TRP | F | 109 | 32.825 | 4.646 | −21.456 | 1.00 | 76.71 | F |
| ATOM | 18882 | CE3 | TRP | F | 109 | 35.153 | 4.043 | −21.755 | 1.00 | 77.56 | F |
| ATOM | 18883 | CD1 | TRP | F | 109 | 32.000 | 2.833 | −20.450 | 1.00 | 75.55 | F |
| ATOM | 18884 | NE1 | TRP | F | 109 | 31.693 | 4.085 | −20.931 | 1.00 | 73.87 | F |
| ATOM | 18885 | CZ2 | TRP | F | 109 | 33.017 | 5.894 | −22.055 | 1.00 | 73.16 | F |
| ATOM | 18886 | CZ3 | TRP | F | 109 | 35.346 | 5.287 | −22.353 | 1.00 | 77.32 | F |
| ATOM | 18887 | CH2 | TRP | F | 109 | 34.281 | 6.196 | −22.497 | 1.00 | 72.96 | F |
| ATOM | 18888 | C | TRP | F | 109 | 35.981 | 2.289 | −19.125 | 1.00 | 94.83 | F |
| ATOM | 18889 | O | TRP | F | 109 | 35.871 | 3.497 | −18.911 | 1.00 | 96.44 | F |
| ATOM | 18890 | N | TYR | F | 110 | 37.115 | 1.710 | −19.499 | 1.00 | 81.53 | F |
| ATOM | 18891 | CA | TYR | F | 110 | 38.338 | 2.468 | −19.711 | 1.00 | 80.17 | F |
| ATOM | 18892 | CB | TYR | F | 110 | 39.422 | 2.026 | −18.733 | 1.00 | 87.33 | F |
| ATOM | 18893 | CG | TYR | F | 110 | 39.100 | 2.282 | −17.281 | 1.00 | 90.88 | F |
| ATOM | 18894 | CD1 | TYR | F | 110 | 38.143 | 1.519 | −16.609 | 1.00 | 94.97 | F |
| ATOM | 18895 | CE1 | TYR | F | 110 | 37.873 | 1.734 | −15.254 | 1.00 | 95.29 | F |
| ATOM | 18896 | CD2 | TYR | F | 110 | 39.776 | 3.272 | −16.567 | 1.00 | 91.11 | F |
| ATOM | 18897 | CE2 | TYR | F | 110 | 39.515 | 3.496 | −15.219 | 1.00 | 96.48 | F |
| ATOM | 18898 | CZ | TYR | F | 110 | 38.568 | 2.727 | −14.567 | 1.00 | 96.77 | F |
| ATOM | 18899 | OH | TYR | F | 110 | 38.338 | 2.947 | −13.230 | 1.00 | 97.97 | F |
| ATOM | 18900 | C | TYR | F | 110 | 38.824 | 2.241 | −21.137 | 1.00 | 78.68 | F |
| ATOM | 18901 | O | TYR | F | 110 | 38.800 | 1.118 | −21.637 | 1.00 | 79.87 | F |
| ATOM | 18902 | N | GLU | F | 111 | 39.270 | 3.312 | −21.782 | 1.00 | 74.12 | F |
| ATOM | 18903 | CA | GLU | F | 111 | 39.751 | 3.235 | −23.153 | 1.00 | 72.14 | F |
| ATOM | 18904 | CB | GLU | F | 111 | 38.612 | 3.564 | −24.116 | 1.00 | 78.25 | F |
| ATOM | 18905 | CG | GLU | F | 111 | 38.585 | 2.682 | −25.327 | 1.00 | 85.17 | F |
| ATOM | 18906 | CD | GLU | F | 111 | 38.390 | 1.228 | −24.965 | 1.00 | 90.14 | F |
| ATOM | 18907 | OE1 | GLU | F | 111 | 37.352 | 0.920 | −24.343 | 1.00 | 90.35 | F |
| ATOM | 18908 | OE2 | GLU | F | 111 | 39.269 | 0.399 | −25.300 | 1.00 | 97.30 | F |
| ATOM | 18909 | C | GLU | F | 111 | 40.905 | 4.211 | −23.376 | 1.00 | 72.72 | F |
| ATOM | 18910 | O | GLU | F | 111 | 40.700 | 5.426 | −23.405 | 1.00 | 78.08 | F |
| ATOM | 18911 | N | PRO | F | 112 | 42.139 | 3.692 | −23.518 | 1.00 | 67.72 | F |
| ATOM | 18912 | CD | PRO | F | 112 | 42.556 | 2.321 | −23.173 | 1.00 | 51.44 | F |
| ATOM | 18913 | CA | PRO | F | 112 | 43.318 | 4.535 | −23.740 | 1.00 | 66.95 | F |
| ATOM | 18914 | CB | PRO | F | 112 | 44.411 | 3.775 | −23.005 | 1.00 | 47.51 | F |
| ATOM | 18915 | CG | PRO | F | 112 | 44.077 | 2.374 | −23.358 | 1.00 | 48.64 | F |
| ATOM | 18916 | C | PRO | F | 112 | 43.639 | 4.676 | −25.227 | 1.00 | 63.98 | F |
| ATOM | 18917 | O | PRO | F | 112 | 43.263 | 3.826 | −26.032 | 1.00 | 64.11 | F |
| ATOM | 18918 | N | ILE | F | 113 | 44.324 | 5.757 | −25.582 | 1.00 | 60.67 | F |
| ATOM | 18919 | CA | ILE | F | 113 | 44.727 | 5.984 | −26.964 | 1.00 | 62.64 | F |
| ATOM | 18920 | CB | ILE | F | 113 | 43.793 | 6.949 | −27.699 | 1.00 | 53.30 | F |
| ATOM | 18921 | CG2 | ILE | F | 113 | 44.303 | 7.142 | −29.116 | 1.00 | 49.32 | F |
| ATOM | 18922 | CG1 | ILE | F | 113 | 42.370 | 6.402 | −27.743 | 1.00 | 50.36 | F |
| ATOM | 18923 | CD1 | ILE | F | 113 | 41.416 | 7.254 | −28.586 | 1.00 | 50.84 | F |
| ATOM | 18924 | C | ILE | F | 113 | 46.121 | 6.599 | −26.999 | 1.00 | 66.86 | F |
| ATOM | 18925 | O | ILE | F | 113 | 46.376 | 7.618 | −26.352 | 1.00 | 70.26 | F |
| ATOM | 18926 | N | TYR | F | 114 | 47.022 | 5.981 | −27.753 | 1.00 | 41.12 | F |
| ATOM | 18927 | CA | TYR | F | 114 | 48.380 | 6.504 | −27.870 | 1.00 | 39.40 | F |
| ATOM | 18928 | CB | TYR | F | 114 | 49.409 | 5.430 | −27.526 | 1.00 | 43.56 | F |
| ATOM | 18929 | CG | TYR | F | 114 | 50.812 | 5.962 | −27.411 | 1.00 | 41.42 | F |
| ATOM | 18930 | CD1 | TYR | F | 114 | 51.259 | 6.540 | −26.231 | 1.00 | 45.71 | F |
| ATOM | 18931 | CE1 | TYR | F | 114 | 52.547 | 7.052 | −26.126 | 1.00 | 45.28 | F |
| ATOM | 18932 | CD2 | TYR | F | 114 | 51.689 | 5.907 | −28.498 | 1.00 | 41.59 | F |
| ATOM | 18933 | CE2 | TYR | F | 114 | 52.978 | 6.420 | −28.414 | 1.00 | 45.92 | F |
| ATOM | 18934 | CZ | TYR | F | 114 | 53.406 | 6.992 | −27.223 | 1.00 | 46.00 | F |
| ATOM | 18935 | OH | TYR | F | 114 | 54.688 | 7.513 | −27.123 | 1.00 | 45.50 | F |
| ATOM | 18936 | C | TYR | F | 114 | 48.628 | 7.005 | −29.294 | 1.00 | 39.24 | F |
| ATOM | 18937 | O | TYR | F | 114 | 48.153 | 6.427 | −30.267 | 1.00 | 42.15 | F |
| ATOM | 18938 | N | LEU | F | 115 | 49.393 | 8.080 | −29.405 | 1.00 | 39.71 | F |
| ATOM | 18939 | CA | LEU | F | 115 | 49.712 | 8.681 | −30.692 | 1.00 | 38.75 | F |
| ATOM | 18940 | CB | LEU | F | 115 | 48.857 | 9.934 | −30.882 | 1.00 | 55.80 | F |
| ATOM | 18941 | CG | LEU | F | 115 | 47.727 | 9.968 | −31.907 | 1.00 | 59.05 | F |
| ATOM | 18942 | CD1 | LEU | F | 115 | 47.003 | 8.636 | −31.981 | 1.00 | 57.43 | F |
| ATOM | 18943 | CD2 | LEU | F | 115 | 46.789 | 11.097 | −31.528 | 1.00 | 62.35 | F |
| ATOM | 18944 | C | LEU | F | 115 | 51.185 | 9.072 | −30.695 | 1.00 | 41.19 | F |
| ATOM | 18945 | O | LEU | F | 115 | 51.697 | 9.572 | −29.691 | 1.00 | 43.72 | F |
| ATOM | 18946 | N | GLY | F | 116 | 51.867 | 8.853 | −31.814 | 1.00 | 39.12 | F |
| ATOM | 18947 | CA | GLY | F | 116 | 53.268 | 9.222 | −31.875 | 1.00 | 33.46 | F |
| ATOM | 18948 | C | GLY | F | 116 | 53.910 | 9.045 | −33.233 | 1.00 | 37.31 | F |
| ATOM | 18949 | O | GLY | F | 116 | 53.627 | 8.077 | −33.943 | 1.00 | 36.19 | F |
| ATOM | 18950 | N | GLY | F | 117 | 54.786 | 9.983 | −33.590 | 1.00 | 34.82 | F |
| ATOM | 18951 | CA | GLY | F | 117 | 55.474 | 9.928 | −34.868 | 1.00 | 37.82 | F |
| ATOM | 18952 | C | GLY | F | 117 | 56.536 | 11.004 | −34.965 | 1.00 | 39.13 | F |
| ATOM | 18953 | O | GLY | F | 117 | 56.597 | 11.895 | −34.120 | 1.00 | 40.51 | F |
| ATOM | 18954 | N | VAL | F | 118 | 57.374 | 10.925 | −35.994 | 1.00 | 32.51 | F |
| ATOM | 18955 | CA | VAL | F | 118 | 58.442 | 11.901 | −36.201 | 1.00 | 35.29 | F |
| ATOM | 18956 | CB | VAL | F | 118 | 59.756 | 11.189 | −36.570 | 1.00 | 33.83 | F |
| ATOM | 18957 | CG1 | VAL | F | 118 | 60.853 | 12.192 | −36.845 | 1.00 | 35.23 | F |
| ATOM | 18958 | CG2 | VAL | F | 118 | 60.158 | 10.284 | −35.427 | 1.00 | 33.89 | F |
| ATOM | 18959 | C | VAL | F | 118 | 58.048 | 12.886 | −37.298 | 1.00 | 33.20 | F |
| ATOM | 18960 | O | VAL | F | 118 | 57.517 | 12.491 | −38.335 | 1.00 | 37.15 | F |
| ATOM | 18961 | N | PHE | F | 119 | 58.312 | 14.171 | −37.057 | 1.00 | 43.29 | F |
| ATOM | 18962 | CA | PHE | F | 119 | 57.952 | 15.224 | −38.000 | 1.00 | 44.60 | F |
| ATOM | 18963 | CB | PHE | F | 119 | 56.607 | 15.844 | −37.590 | 1.00 | 30.77 | F |
| ATOM | 18964 | CG | PHE | F | 119 | 55.459 | 14.883 | −37.609 | 1.00 | 28.37 | F |
| ATOM | 18965 | CD1 | PHE | F | 119 | 55.214 | 14.060 | −36.526 | 1.00 | 26.56 | F |
| ATOM | 18966 | CD2 | PHE | F | 119 | 54.648 | 14.769 | −38.728 | 1.00 | 27.05 | F |
| ATOM | 18967 | CE1 | PHE | F | 119 | 54.173 | 13.123 | −36.553 | 1.00 | 27.63 | F |
| ATOM | 18968 | CE2 | PHE | F | 119 | 53.600 | 13.832 | −38.765 | 1.00 | 27.86 | F |
| ATOM | 18969 | CZ | PHE | F | 119 | 53.365 | 13.010 | −37.678 | 1.00 | 26.95 | F |
| ATOM | 18970 | C | PHE | F | 119 | 58.967 | 16.358 | −38.160 | 1.00 | 45.51 | F |
| ATOM | 18971 | O | PHE | F | 119 | 59.680 | 16.704 | −37.227 | 1.00 | 41.61 | F |
| ATOM | 18972 | N | GLN | F | 120 | 59.010 | 16.932 | −39.360 | 1.00 | 36.85 | F |
| ATOM | 18973 | CA | GLN | F | 120 | 59.877 | 18.068 | −39.683 | 1.00 | 40.22 | F |
| ATOM | 18974 | CB | GLN | F | 120 | 60.098 | 18.144 | −41.195 | 1.00 | 66.06 | F |
| ATOM | 18975 | CG | GLN | F | 120 | 61.535 | 18.069 | −41.654 | 1.00 | 66.21 | F |
| ATOM | 18976 | CD | GLN | F | 120 | 62.420 | 19.098 | −40.989 | 1.00 | 65.81 | F |
| ATOM | 18977 | OE1 | GLN | F | 120 | 62.025 | 20.243 | −40.808 | 1.00 | 65.37 | F |
| ATOM | 18978 | NE2 | GLN | F | 120 | 63.634 | 18.695 | −40.632 | 1.00 | 68.64 | F |
| ATOM | 18979 | C | GLN | F | 120 | 59.079 | 19.304 | −39.235 | 1.00 | 39.36 | F |
| ATOM | 18980 | O | GLN | F | 120 | 57.953 | 19.516 | −39.695 | 1.00 | 42.06 | F |
| ATOM | 18981 | N | LEU | F | 121 | 59.648 | 20.111 | −38.343 | 1.00 | 32.02 | F |
| ATOM | 18982 | CA | LEU | F | 121 | 58.969 | 21.305 | −37.834 | 1.00 | 33.68 | F |
| ATOM | 18983 | CB | LEU | F | 121 | 58.671 | 21.133 | −36.343 | 1.00 | 28.55 | F |
| ATOM | 18984 | CG | LEU | F | 121 | 57.324 | 20.542 | −35.927 | 1.00 | 26.82 | F |
| ATOM | 18985 | CD1 | LEU | F | 121 | 56.804 | 19.581 | −36.969 | 1.00 | 28.21 | F |
| ATOM | 18986 | CD2 | LEU | F | 121 | 57.485 | 19.870 | −34.559 | 1.00 | 22.31 | F |
| ATOM | 18987 | C | LEU | F | 121 | 59.775 | 22.580 | −38.035 | 1.00 | 35.72 | F |
| ATOM | 18988 | O | LEU | F | 121 | 60.999 | 22.549 | −38.047 | 1.00 | 40.99 | F |
| ATOM | 18989 | N | GLU | F | 122 | 59.069 | 23.699 | −38.186 | 1.00 | 50.02 | F |
| ATOM | 18990 | CA | GLU | F | 122 | 59.691 | 25.014 | −38.377 | 1.00 | 51.56 | F |
| ATOM | 18991 | CB | GLU | F | 122 | 58.860 | 25.906 | −39.309 | 1.00 | 87.61 | F |
| ATOM | 18992 | CG | GLU | F | 122 | 58.622 | 25.438 | −40.725 | 1.00 | 90.65 | F |
| ATOM | 18993 | CD | GLU | F | 122 | 57.667 | 26.369 | −41.474 | 1.00 | 95.93 | F |
| ATOM | 18994 | OE1 | GLU | F | 122 | 56.497 | 26.503 | −41.051 | 1.00 | 97.43 | F |
| ATOM | 18995 | OE2 | GLU | F | 122 | 58.089 | 26.975 | −42.481 | 1.00 | 96.02 | F |
| ATOM | 18996 | C | GLU | F | 122 | 59.745 | 25.740 | −37.040 | 1.00 | 50.36 | F |
| ATOM | 18997 | O | GLU | F | 122 | 58.913 | 25.507 | −36.164 | 1.00 | 51.55 | F |
| ATOM | 18998 | N | LYS | F | 123 | 60.707 | 26.639 | −36.888 | 1.00 | 48.42 | F |
| ATOM | 18999 | CA | LYS | F | 123 | 60.780 | 27.417 | −35.663 | 1.00 | 49.27 | F |
| ATOM | 19000 | CB | LYS | F | 123 | 61.964 | 28.378 | −35.705 | 1.00 | 68.86 | F |
| ATOM | 19001 | CG | LYS | F | 123 | 61.964 | 29.361 | −34.560 | 1.00 | 65.02 | F |
| ATOM | 19002 | CD | LYS | F | 123 | 63.096 | 30.356 | −34.691 | 1.00 | 62.69 | F |
| ATOM | 19003 | CE | LYS | F | 123 | 63.057 | 31.386 | −33.567 | 1.00 | 64.42 | F |
| ATOM | 19004 | NZ | LYS | F | 123 | 64.118 | 32.422 | −33.748 | 1.00 | 68.43 | F |
| ATOM | 19005 | C | LYS | F | 123 | 59.475 | 28.212 | −35.592 | 1.00 | 50.09 | F |
| ATOM | 19006 | O | LYS | F | 123 | 59.103 | 28.893 | −36.549 | 1.00 | 54.85 | F |
| ATOM | 19007 | N | GLY | F | 124 | 58.769 | 28.105 | −34.473 | 1.00 | 49.01 | F |
| ATOM | 19008 | CA | GLY | F | 124 | 57.524 | 28.828 | −34.327 | 1.00 | 50.12 | F |
| ATOM | 19009 | C | GLY | F | 124 | 56.266 | 27.997 | −34.467 | 1.00 | 51.53 | F |
| ATOM | 19010 | O | GLY | F | 124 | 55.182 | 28.482 | −34.146 | 1.00 | 52.33 | F |
| ATOM | 19011 | N | ASP | F | 125 | 56.384 | 26.759 | −34.943 | 1.00 | 46.33 | F |
| ATOM | 19012 | CA | ASP | F | 125 | 55.204 | 25.907 | −35.105 | 1.00 | 45.12 | F |
| ATOM | 19013 | CB | ASP | F | 125 | 55.583 | 24.567 | −35.751 | 1.00 | 66.43 | F |
| ATOM | 19014 | CG | ASP | F | 125 | 55.962 | 24.701 | −37.217 | 1.00 | 71.89 | F |
| ATOM | 19015 | OD1 | ASP | F | 125 | 55.733 | 25.779 | −37.806 | 1.00 | 73.54 | F |
| ATOM | 19016 | OD2 | ASP | F | 125 | 56.479 | 23.720 | −37.784 | 1.00 | 70.55 | F |
| ATOM | 19017 | C | ASP | F | 125 | 54.490 | 25.643 | −33.774 | 1.00 | 44.17 | F |
| ATOM | 19018 | O | ASP | F | 125 | 55.123 | 25.574 | −32.722 | 1.00 | 41.08 | F |
| ATOM | 19019 | N | ARG | F | 126 | 53.171 | 25.495 | −33.836 | 1.00 | 52.33 | F |
| ATOM | 19020 | CA | ARG | F | 126 | 52.359 | 25.241 | −32.655 | 1.00 | 47.81 | F |
| ATOM | 19021 | CB | ARG | F | 126 | 51.221 | 26.261 | −32.568 | 1.00 | 97.09 | F |
| ATOM | 19022 | CG | ARG | F | 126 | 51.640 | 27.684 | −32.263 | 1.00 | 95.58 | F |
| ATOM | 19023 | CD | ARG | F | 126 | 50.456 | 28.639 | −32.420 | 1.00 | 100.50 | F |
| ATOM | 19024 | NE | ARG | F | 126 | 50.714 | 29.931 | −31.786 | 1.00 | 104.05 | F |
| ATOM | 19025 | CZ | ARG | F | 126 | 50.632 | 30.160 | −30.473 | 1.00 | 107.28 | F |
| ATOM | 19026 | NH1 | ARG | F | 126 | 50.895 | 31.370 | −29.986 | 1.00 | 106.95 | F |
| ATOM | 19027 | NH2 | ARG | F | 126 | 50.268 | 29.189 | −29.641 | 1.00 | 105.47 | F |
| ATOM | 19028 | C | ARG | F | 126 | 51.757 | 23.845 | −32.738 | 1.00 | 48.65 | F |
| ATOM | 19029 | O | ARG | F | 126 | 51.093 | 23.521 | −33.721 | 1.00 | 45.87 | F |
| ATOM | 19030 | N | LEU | F | 127 | 51.968 | 23.026 | −31.710 | 1.00 | 45.08 | F |
| ATOM | 19031 | CA | LEU | F | 127 | 51.430 | 21.671 | −31.702 | 1.00 | 41.46 | F |
| ATOM | 19032 | CB | LEU | F | 127 | 52.530 | 20.679 | −31.352 | 1.00 | 35.89 | F |
| ATOM | 19033 | CG | LEU | F | 127 | 53.687 | 20.709 | −32.344 | 1.00 | 34.87 | F |
| ATOM | 19034 | CD1 | LEU | F | 127 | 54.873 | 19.962 | −31.782 | 1.00 | 35.45 | F |
| ATOM | 19035 | CD2 | LEU | F | 127 | 53.233 | 20.106 | −33.673 | 1.00 | 35.53 | F |
| ATOM | 19036 | C | LEU | F | 127 | 50.305 | 21.567 | −30.696 | 1.00 | 41.21 | F |
| ATOM | 19037 | O | LEU | F | 127 | 50.422 | 22.074 | −29.583 | 1.00 | 37.41 | F |
| ATOM | 19038 | N | SER | F | 128 | 49.221 | 20.898 | −31.078 | 1.00 | 43.09 | F |
| ATOM | 19039 | CA | SER | F | 128 | 48.064 | 20.756 | −30.200 | 1.00 | 42.76 | F |
| ATOM | 19040 | CB | SER | F | 128 | 46.842 | 21.458 | −30.801 | 1.00 | 94.89 | F |
| ATOM | 19041 | OG | SER | F | 128 | 47.186 | 22.703 | −31.382 | 1.00 | 100.74 | F |
| ATOM | 19042 | C | SER | F | 128 | 47.729 | 19.297 | −30.018 | 1.00 | 41.94 | F |
| ATOM | 19043 | O | SER | F | 128 | 47.825 | 18.520 | −30.958 | 1.00 | 43.22 | F |
| ATOM | 19044 | N | ALA | F | 129 | 47.334 | 18.930 | −28.807 | 1.00 | 58.41 | F |
| ATOM | 19045 | CA | ALA | F | 129 | 46.934 | 17.566 | −28.505 | 1.00 | 59.38 | F |
| ATOM | 19046 | CB | ALA | F | 129 | 47.879 | 16.962 | −27.484 | 1.00 | 41.04 | F |
| ATOM | 19047 | C | ALA | F | 129 | 45.519 | 17.698 | −27.939 | 1.00 | 58.15 | F |
| ATOM | 19048 | O | ALA | F | 129 | 45.337 | 17.996 | −26.765 | 1.00 | 59.03 | F |
| ATOM | 19049 | N | GLU | F | 130 | 44.515 | 17.488 | −28.779 | 1.00 | 49.46 | F |
| ATOM | 19050 | CA | GLU | F | 130 | 43.127 | 17.636 | −28.351 | 1.00 | 50.43 | F |
| ATOM | 19051 | CB | GLU | F | 130 | 42.476 | 18.734 | −29.190 | 1.00 | 81.32 | F |
| ATOM | 19052 | CG | GLU | F | 130 | 43.193 | 20.070 | −29.063 | 1.00 | 80.86 | F |
| ATOM | 19053 | CD | GLU | F | 130 | 42.928 | 21.011 | −30.229 | 1.00 | 86.35 | F |
| ATOM | 19054 | OE1 | GLU | F | 130 | 43.413 | 22.166 | −30.192 | 1.00 | 87.72 | F |
| ATOM | 19055 | OE2 | GLU | F | 130 | 42.244 | 20.596 | −31.186 | 1.00 | 86.16 | F |
| ATOM | 19056 | C | GLU | F | 130 | 42.263 | 16.375 | −28.389 | 1.00 | 46.82 | F |
| ATOM | 19057 | O | GLU | F | 130 | 42.608 | 15.381 | −29.020 | 1.00 | 46.74 | F |
| ATOM | 19058 | N | ILE | F | 131 | 41.143 | 16.421 | −27.679 | 1.00 | 59.24 | F |
| ATOM | 19059 | CA | ILE | F | 131 | 40.196 | 15.311 | −27.643 | 1.00 | 61.78 | F |
| ATOM | 19060 | CB | ILE | F | 131 | 40.203 | 14.580 | −26.300 | 1.00 | 62.37 | F |
| ATOM | 19061 | CG2 | ILE | F | 131 | 41.533 | 13.880 | −26.096 | 1.00 | 62.60 | F |
| ATOM | 19062 | CG1 | ILE | F | 131 | 39.899 | 15.572 | −25.182 | 1.00 | 58.56 | F |
| ATOM | 19063 | CD1 | ILE | F | 131 | 39.806 | 14.927 | −23.810 | 1.00 | 57.17 | F |
| ATOM | 19064 | C | ILE | F | 131 | 38.816 | 15.917 | −27.849 | 1.00 | 63.67 | F |
| ATOM | 19065 | O | ILE | F | 131 | 38.637 | 17.116 | −27.671 | 1.00 | 64.39 | F |
| ATOM | 19066 | N | ASN | F | 132 | 37.843 | 15.094 | −28.217 | 1.00 | 67.90 | F |
| ATOM | 19067 | CA | ASN | F | 132 | 36.495 | 15.585 | −28.464 | 1.00 | 64.90 | F |
| ATOM | 19068 | CB | ASN | F | 132 | 35.896 | 14.829 | −29.643 | 1.00 | 71.52 | F |
| ATOM | 19069 | CG | ASN | F | 132 | 35.683 | 13.364 | −29.333 | 1.00 | 70.22 | F |
| ATOM | 19070 | OD1 | ASN | F | 132 | 36.529 | 12.721 | −28.715 | 1.00 | 71.91 | F |
| ATOM | 19071 | ND2 | ASN | F | 132 | 34.553 | 12.824 | −29.768 | 1.00 | 68.05 | F |
| ATOM | 19072 | C | ASN | F | 132 | 35.559 | 15.477 | −27.256 | 1.00 | 62.55 | F |
| ATOM | 19073 | O | ASN | F | 132 | 34.622 | 16.265 | −27.133 | 1.00 | 65.72 | F |
| ATOM | 19074 | N | ARG | F | 133 | 35.804 | 14.512 | −26.368 | 1.00 | 60.81 | F |
| ATOM | 19075 | CA | ARG | F | 133 | 34.958 | 14.326 | −25.188 | 1.00 | 56.40 | F |
| ATOM | 19076 | CB | ARG | F | 133 | 34.298 | 12.944 | −25.222 | 1.00 | 99.85 | F |
| ATOM | 19077 | CG | ARG | F | 133 | 33.383 | 12.719 | −26.410 | 1.00 | 102.43 | F |
| ATOM | 19078 | CD | ARG | F | 133 | 32.288 | 13.773 | −26.466 | 1.00 | 107.67 | F |
| ATOM | 19079 | NE | ARG | F | 133 | 31.448 | 13.766 | −25.270 | 1.00 | 105.53 | F |
| ATOM | 19080 | CZ | ARG | F | 133 | 30.575 | 12.808 | −24.966 | 1.00 | 106.63 | F |
| ATOM | 19081 | NH1 | ARG | F | 133 | 29.862 | 12.895 | −23.852 | 1.00 | 109.31 | F |
| ATOM | 19082 | NH2 | ARG | F | 133 | 30.405 | 11.768 | −25.774 | 1.00 | 105.92 | F |
| ATOM | 19083 | C | ARG | F | 133 | 35.707 | 14.486 | −23.864 | 1.00 | 60.99 | F |
| ATOM | 19084 | O | ARG | F | 133 | 35.885 | 13.519 | −23.122 | 1.00 | 63.35 | F |
| ATOM | 19085 | N | PRO | F | 134 | 36.136 | 15.714 | −23.542 | 1.00 | 62.18 | F |
| ATOM | 19086 | CD | PRO | F | 134 | 35.863 | 16.967 | −24.261 | 1.00 | 69.35 | F |
| ATOM | 19087 | CA | PRO | F | 134 | 36.865 | 15.987 | −22.301 | 1.00 | 65.90 | F |
| ATOM | 19088 | CB | PRO | F | 134 | 37.152 | 17.481 | −22.391 | 1.00 | 72.07 | F |
| ATOM | 19089 | CG | PRO | F | 134 | 35.993 | 17.990 | −23.154 | 1.00 | 74.07 | F |
| ATOM | 19090 | C | PRO | F | 134 | 36.074 | 15.622 | −21.050 | 1.00 | 71.08 | F |
| ATOM | 19091 | O | PRO | F | 134 | 36.615 | 15.589 | −19.949 | 1.00 | 74.12 | F |
| ATOM | 19092 | N | ASP | F | 135 | 34.789 | 15.347 | −21.213 | 1.00 | 85.41 | F |
| ATOM | 19093 | CA | ASP | F | 135 | 33.973 | 14.977 | −20.071 | 1.00 | 81.87 | F |
| ATOM | 19094 | CB | ASP | F | 135 | 32.495 | 15.191 | −20.401 | 1.00 | 98.80 | F |
| ATOM | 19095 | CG | ASP | F | 135 | 32.136 | 14.730 | −21.797 | 1.00 | 98.27 | F |
| ATOM | 19096 | OD1 | ASP | F | 135 | 32.043 | 13.502 | −22.015 | 1.00 | 94.21 | F |
| ATOM | 19097 | OD2 | ASP | F | 135 | 31.955 | 15.599 | −22.681 | 1.00 | 99.85 | F |
| ATOM | 19098 | C | ASP | F | 135 | 34.239 | 13.531 | −19.645 | 1.00 | 80.58 | F |
| ATOM | 19099 | O | ASP | F | 135 | 33.833 | 13.115 | −18.566 | 1.00 | 87.27 | F |
| ATOM | 19100 | N | TYR | F | 136 | 34.937 | 12.773 | −20.486 | 1.00 | 73.86 | F |
| ATOM | 19101 | CA | TYR | F | 136 | 35.260 | 11.378 | −20.186 | 1.00 | 75.07 | F |
| ATOM | 19102 | CB | TYR | F | 136 | 34.970 | 10.491 | −21.396 | 1.00 | 93.63 | F |
| ATOM | 19103 | CG | TYR | F | 136 | 33.513 | 10.207 | −21.656 | 1.00 | 93.24 | F |
| ATOM | 19104 | CD1 | TYR | F | 136 | 32.919 | 10.573 | −22.864 | 1.00 | 92.84 | F |
| ATOM | 19105 | CE1 | TYR | F | 136 | 31.590 | 10.257 | −23.141 | 1.00 | 95.39 | F |
| ATOM | 19106 | CD2 | TYR | F | 136 | 32.741 | 9.522 | −20.722 | 1.00 | 95.41 | F |
| ATOM | 19107 | CE2 | TYR | F | 136 | 31.411 | 9.201 | −20.988 | 1.00 | 94.88 | F |
| ATOM | 19108 | CZ | TYR | F | 136 | 30.844 | 9.572 | −22.200 | 1.00 | 96.18 | F |
| ATOM | 19109 | OH | TYR | F | 136 | 29.537 | 9.253 | −22.478 | 1.00 | 100.02 | F |
| ATOM | 19110 | C | TYR | F | 136 | 36.738 | 11.228 | −19.822 | 1.00 | 76.98 | F |
| ATOM | 19111 | O | TYR | F | 136 | 37.239 | 10.117 | −19.632 | 1.00 | 79.86 | F |
| ATOM | 19112 | N | LEU | F | 137 | 37.436 | 12.351 | −19.731 | 1.00 | 69.45 | F |
| ATOM | 19113 | CA | LEU | F | 137 | 38.853 | 12.338 | −19.416 | 1.00 | 66.16 | F |
| ATOM | 19114 | CB | LEU | F | 137 | 39.421 | 13.746 | −19.535 | 1.00 | 75.07 | F |
| ATOM | 19115 | CG | LEU | F | 137 | 40.533 | 13.973 | −20.551 | 1.00 | 72.80 | F |
| ATOM | 19116 | CD1 | LEU | F | 137 | 41.114 | 15.357 | −20.326 | 1.00 | 74.94 | F |
| ATOM | 19117 | CD2 | LEU | F | 137 | 41.613 | 12.923 | −20.400 | 1.00 | 76.56 | F |
| ATOM | 19118 | C | LEU | F | 137 | 39.177 | 11.814 | −18.026 | 1.00 | 65.53 | F |
| ATOM | 19119 | O | LEU | F | 137 | 38.773 | 12.417 | −17.022 | 1.00 | 64.38 | F |
| ATOM | 19120 | N | LEU | F | 138 | 39.909 | 10.701 | −17.954 | 1.00 | 66.70 | F |
| ATOM | 19121 | CA | LEU | F | 138 | 40.313 | 10.166 | −16.652 | 1.00 | 64.50 | F |
| ATOM | 19122 | CB | LEU | F | 138 | 40.189 | 8.638 | −16.578 | 1.00 | 92.83 | F |
| ATOM | 19123 | CG | LEU | F | 138 | 40.880 | 8.063 | −15.322 | 1.00 | 88.95 | F |
| ATOM | 19124 | CD1 | LEU | F | 138 | 40.432 | 8.826 | −14.086 | 1.00 | 90.52 | F |
| ATOM | 19125 | CD2 | LEU | F | 138 | 40.578 | 6.589 | −15.169 | 1.00 | 91.87 | F |
| ATOM | 19126 | C | LEU | F | 138 | 41.751 | 10.550 | −16.328 | 1.00 | 67.85 | F |
| ATOM | 19127 | O | LEU | F | 138 | 42.700 | 9.896 | −16.762 | 1.00 | 67.00 | F |
| ATOM | 19128 | N | PHE | F | 139 | 41.905 | 11.619 | −15.562 | 1.00 | 74.32 | F |
| ATOM | 19129 | CA | PHE | F | 139 | 43.225 | 12.069 | −15.167 | 1.00 | 75.70 | F |
| ATOM | 19130 | CB | PHE | F | 139 | 43.472 | 13.497 | −15.670 | 1.00 | 77.60 | F |
| ATOM | 19131 | CG | PHE | F | 139 | 42.316 | 14.436 | −15.454 | 1.00 | 76.75 | F |
| ATOM | 19132 | CD1 | PHE | F | 139 | 42.127 | 15.516 | −16.307 | 1.00 | 76.28 | F |
| ATOM | 19133 | CD2 | PHE | F | 139 | 41.432 | 14.259 | −14.394 | 1.00 | 79.36 | F |
| ATOM | 19134 | CE1 | PHE | F | 139 | 41.081 | 16.403 | −16.112 | 1.00 | 80.92 | F |
| ATOM | 19135 | CE2 | PHE | F | 139 | 40.382 | 15.142 | −14.191 | 1.00 | 76.90 | F |
| ATOM | 19136 | CZ | PHE | F | 139 | 40.206 | 16.217 | −15.053 | 1.00 | 80.84 | F |
| ATOM | 19137 | C | PHE | F | 139 | 43.330 | 11.987 | −13.646 | 1.00 | 79.73 | F |
| ATOM | 19138 | O | PHE | F | 139 | 43.919 | 12.856 | −12.995 | 1.00 | 79.83 | F |
| ATOM | 19139 | N | ALA | F | 140 | 42.756 | 10.917 | −13.094 | 1.00 | 76.61 | F |
| ATOM | 19140 | CA | ALA | F | 140 | 42.750 | 10.688 | −11.654 | 1.00 | 77.91 | F |
| ATOM | 19141 | CB | ALA | F | 140 | 41.625 | 9.737 | −11.289 | 1.00 | 66.20 | F |
| ATOM | 19142 | C | ALA | F | 140 | 44.064 | 10.141 | −11.128 | 1.00 | 78.30 | F |
| ATOM | 19143 | O | ALA | F | 140 | 44.581 | 10.618 | −10.120 | 1.00 | 80.19 | F |
| ATOM | 19144 | N | GLU | F | 141 | 44.601 | 9.139 | −11.813 | 1.00 | 87.14 | F |
| ATOM | 19145 | CA | GLU | F | 141 | 45.844 | 8.523 | −11.381 | 1.00 | 90.32 | F |
| ATOM | 19146 | CB | GLU | F | 141 | 46.075 | 7.197 | −12.119 | 1.00 | 175.23 | F |
| ATOM | 19147 | CG | GLU | F | 141 | 47.283 | 6.403 | −11.617 | 1.00 | 175.23 | F |
| ATOM | 19148 | CD | GLU | F | 141 | 48.598 | 6.841 | −12.250 | 1.00 | 175.23 | F |
| ATOM | 19149 | OE1 | GLU | F | 141 | 49.659 | 6.642 | −11.621 | 1.00 | 175.23 | F |
| ATOM | 19150 | OE2 | GLU | F | 141 | 48.577 | 7.370 | −13.381 | 1.00 | 175.23 | F |
| ATOM | 19151 | C | GLU | F | 141 | 47.069 | 9.414 | −11.522 | 1.00 | 92.42 | F |
| ATOM | 19152 | O | GLU | F | 141 | 47.479 | 10.046 | −10.553 | 1.00 | 96.10 | F |
| ATOM | 19153 | N | SER | F | 142 | 47.656 | 9.481 | −12.713 | 1.00 | 76.54 | F |
| ATOM | 19154 | CA | SER | F | 142 | 48.861 | 10.284 | −12.869 | 1.00 | 79.53 | F |
| ATOM | 19155 | CB | SER | F | 142 | 49.909 | 9.789 | −11.879 | 1.00 | 52.01 | F |
| ATOM | 19156 | OG | SER | F | 142 | 50.957 | 10.718 | −11.735 | 1.00 | 52.89 | F |
| ATOM | 19157 | C | SER | F | 142 | 49.464 | 10.280 | −14.279 | 1.00 | 80.85 | F |
| ATOM | 19158 | O | SER | F | 142 | 49.310 | 11.243 | −15.047 | 1.00 | 85.94 | F |
| ATOM | 19159 | N | GLY | F | 143 | 50.177 | 9.210 | −14.616 | 1.00 | 59.44 | F |
| ATOM | 19160 | CA | GLY | F | 143 | 50.766 | 9.148 | −15.942 | 1.00 | 56.91 | F |
| ATOM | 19161 | C | GLY | F | 143 | 49.787 | 8.588 | −16.955 | 1.00 | 54.98 | F |
| ATOM | 19162 | O | GLY | F | 143 | 50.192 | 7.831 | −17.832 | 1.00 | 56.97 | F |
| ATOM | 19163 | N | GLN | F | 144 | 48.509 | 8.962 | −16.838 | 1.00 | 55.15 | F |
| ATOM | 19164 | CA | GLN | F | 144 | 47.448 | 8.479 | −17.730 | 1.00 | 49.68 | F |
| ATOM | 19165 | CB | GLN | F | 144 | 46.152 | 8.264 | −16.947 | 1.00 | 87.92 | F |
| ATOM | 19166 | CG | GLN | F | 144 | 46.324 | 7.501 | −15.653 | 1.00 | 87.20 | F |
| ATOM | 19167 | CD | GLN | F | 144 | 45.001 | 7.189 | −14.983 | 1.00 | 87.17 | F |
| ATOM | 19168 | OE1 | GLN | F | 144 | 44.146 | 8.064 | −14.824 | 1.00 | 92.74 | F |
| ATOM | 19169 | NE2 | GLN | F | 144 | 44.831 | 5.940 | −14.574 | 1.00 | 85.71 | F |
| ATOM | 19170 | C | GLN | F | 144 | 47.149 | 9.421 | −18.892 | 1.00 | 51.48 | F |
| ATOM | 19171 | O | GLN | F | 144 | 46.568 | 9.018 | −19.896 | 1.00 | 53.79 | F |
| ATOM | 19172 | N | VAL | F | 145 | 47.532 | 10.680 | −18.745 | 1.00 | 36.31 | F |
| ATOM | 19173 | CA | VAL | F | 145 | 47.292 | 11.666 | −19.781 | 1.00 | 34.56 | F |
| ATOM | 19174 | CB | VAL | F | 145 | 46.059 | 12.545 | −19.442 | 1.00 | 32.26 | F |
| ATOM | 19175 | CG1 | VAL | F | 145 | 45.817 | 13.555 | −20.566 | 1.00 | 30.42 | F |
| ATOM | 19176 | CG2 | VAL | F | 145 | 44.836 | 11.666 | −19.236 | 1.00 | 29.34 | F |
| ATOM | 19177 | C | VAL | F | 145 | 48.517 | 12.556 | −19.914 | 1.00 | 35.67 | F |
| ATOM | 19178 | O | VAL | F | 145 | 48.851 | 13.301 | −18.997 | 1.00 | 38.35 | F |
| ATOM | 19179 | N | TYR | F | 146 | 49.187 | 12.462 | −21.059 | 1.00 | 44.79 | F |
| ATOM | 19180 | CA | TYR | F | 146 | 50.381 | 13.259 | −21.320 | 1.00 | 43.00 | F |
| ATOM | 19181 | CB | TYR | F | 146 | 51.647 | 12.534 | −20.819 | 1.00 | 54.68 | F |
| ATOM | 19182 | CG | TYR | F | 146 | 51.761 | 11.100 | −21.268 | 1.00 | 54.71 | F |
| ATOM | 19183 | CD1 | TYR | F | 146 | 51.121 | 10.078 | −20.567 | 1.00 | 54.10 | F |
| ATOM | 19184 | CE1 | TYR | F | 146 | 51.180 | 8.754 | −21.007 | 1.00 | 52.89 | F |
| ATOM | 19185 | CD2 | TYR | F | 146 | 52.468 | 10.768 | −22.424 | 1.00 | 50.30 | F |
| ATOM | 19186 | CE2 | TYR | F | 146 | 52.533 | 9.451 | −22.874 | 1.00 | 52.35 | F |
| ATOM | 19187 | CZ | TYR | F | 146 | 51.888 | 8.447 | −22.162 | 1.00 | 52.39 | F |
| ATOM | 19188 | OH | TYR | F | 146 | 51.951 | 7.144 | −22.599 | 1.00 | 53.37 | F |
| ATOM | 19189 | C | TYR | F | 146 | 50.541 | 13.631 | −22.800 | 1.00 | 41.31 | F |
| ATOM | 19190 | O | TYR | F | 146 | 49.889 | 13.073 | −23.693 | 1.00 | 39.48 | F |
| ATOM | 19191 | N | PHE | F | 147 | 51.439 | 14.578 | −23.030 | 1.00 | 44.32 | F |
| ATOM | 19192 | CA | PHE | F | 147 | 51.709 | 15.110 | −24.343 | 1.00 | 43.84 | F |
| ATOM | 19193 | CB | PHE | F | 147 | 50.713 | 16.242 | −24.593 | 1.00 | 39.46 | F |
| ATOM | 19194 | CG | PHE | F | 147 | 50.983 | 17.062 | −25.822 | 1.00 | 38.51 | F |
| ATOM | 19195 | CD1 | PHE | F | 147 | 51.479 | 16.478 | −26.991 | 1.00 | 39.00 | F |
| ATOM | 19196 | CD2 | PHE | F | 147 | 50.667 | 18.427 | −25.831 | 1.00 | 40.84 | F |
| ATOM | 19197 | CE1 | PHE | F | 147 | 51.653 | 17.244 | −28.153 | 1.00 | 39.33 | F |
| ATOM | 19198 | CE2 | PHE | F | 147 | 50.836 | 19.199 | −26.986 | 1.00 | 39.41 | F |
| ATOM | 19199 | CZ | PHE | F | 147 | 51.331 | 18.604 | −28.150 | 1.00 | 40.25 | F |
| ATOM | 19200 | C | PHE | F | 147 | 53.147 | 15.618 | −24.297 | 1.00 | 45.38 | F |
| ATOM | 19201 | O | PHE | F | 147 | 53.527 | 16.345 | −23.384 | 1.00 | 42.86 | F |
| ATOM | 19202 | N | GLY | F | 148 | 53.962 | 15.214 | −25.262 | 1.00 | 35.59 | F |
| ATOM | 19203 | CA | GLY | F | 148 | 55.336 | 15.669 | −25.268 | 1.00 | 40.07 | F |
| ATOM | 19204 | C | GLY | F | 148 | 56.002 | 15.629 | −26.625 | 1.00 | 42.29 | F |
| ATOM | 19205 | O | GLY | F | 148 | 55.461 | 15.100 | −27.592 | 1.00 | 44.90 | F |
| ATOM | 19206 | N | ILE | F | 149 | 57.185 | 16.221 | −26.688 | 1.00 | 47.63 | F |
| ATOM | 19207 | CA | ILE | F | 149 | 57.980 | 16.256 | −27.901 | 1.00 | 46.04 | F |
| ATOM | 19208 | CB | ILE | F | 149 | 57.615 | 17.446 | −28.810 | 1.00 | 59.12 | F |
| ATOM | 19209 | CG2 | ILE | F | 149 | 56.126 | 17.502 | −29.026 | 1.00 | 59.67 | F |
| ATOM | 19210 | CG1 | ILE | F | 149 | 58.051 | 18.755 | −28.176 | 1.00 | 55.16 | F |
| ATOM | 19211 | CD1 | ILE | F | 149 | 57.846 | 19.932 | −29.096 | 1.00 | 58.41 | F |
| ATOM | 19212 | C | ILE | F | 149 | 59.446 | 16.374 | −27.514 | 1.00 | 48.08 | F |
| ATOM | 19213 | O | ILE | F | 149 | 59.794 | 16.955 | −26.494 | 1.00 | 53.25 | F |
| ATOM | 19214 | N | ILE | F | 150 | 60.302 | 15.806 | −28.338 | 1.00 | 42.07 | F |
| ATOM | 19215 | CA | ILE | F | 150 | 61.731 | 15.836 | −28.100 | 1.00 | 42.58 | F |
| ATOM | 19216 | CB | ILE | F | 150 | 62.204 | 14.497 | −27.468 | 1.00 | 44.01 | F |
| ATOM | 19217 | CG2 | ILE | F | 150 | 61.753 | 13.330 | −28.313 | 1.00 | 47.31 | F |
| ATOM | 19218 | CG1 | ILE | F | 150 | 63.727 | 14.480 | −27.329 | 1.00 | 42.00 | F |
| ATOM | 19219 | CD1 | ILE | F | 150 | 64.245 | 15.403 | −26.283 | 1.00 | 40.01 | F |
| ATOM | 19220 | C | ILE | F | 150 | 62.390 | 16.048 | −29.464 | 1.00 | 43.92 | F |
| ATOM | 19221 | O | ILE | F | 150 | 61.995 | 15.430 | −30.459 | 1.00 | 43.03 | F |
| ATOM | 19222 | N | ALA | F | 151 | 63.379 | 16.934 | −29.517 | 1.00 | 48.97 | F |
| ATOM | 19223 | CA | ALA | F | 151 | 64.069 | 17.208 | −30.769 | 1.00 | 47.71 | F |
| ATOM | 19224 | CB | ALA | F | 151 | 64.736 | 18.571 | −30.706 | 1.00 | 27.39 | F |
| ATOM | 19225 | C | ALA | F | 151 | 65.108 | 16.125 | −31.040 | 1.00 | 48.67 | F |
| ATOM | 19226 | O | ALA | F | 151 | 65.988 | 15.869 | −30.216 | 1.00 | 51.05 | F |
| ATOM | 19227 | N | LEU | F | 152 | 65.000 | 15.484 | −32.197 | 1.00 | 80.95 | F |
| ATOM | 19228 | CA | LEU | F | 152 | 65.938 | 14.435 | −32.562 | 1.00 | 84.93 | F |
| ATOM | 19229 | CB | LEU | F | 152 | 65.183 | 13.234 | −33.122 | 1.00 | 72.73 | F |
| ATOM | 19230 | CG | LEU | F | 152 | 64.196 | 12.599 | −32.145 | 1.00 | 73.98 | F |
| ATOM | 19231 | CD1 | LEU | F | 152 | 63.476 | 11.453 | −32.815 | 1.00 | 71.67 | F |
| ATOM | 19232 | CD2 | LEU | F | 152 | 64.937 | 12.114 | −30.915 | 1.00 | 73.18 | F |
| ATOM | 19233 | C | LEU | F | 152 | 66.944 | 14.936 | −33.591 | 1.00 | 87.78 | F |
| ATOM | 19234 | O | LEU | F | 152 | 68.127 | 15.096 | −33.287 | 1.00 | 93.64 | F |
| ATOM | 19235 | N | ILE | F | 153 | 49.352 | 27.229 | −4.252 | 1.00 | 80.49 | F |
| ATOM | 19236 | CA | ILE | F | 153 | 49.297 | 26.441 | −3.028 | 1.00 | 80.49 | F |
| ATOM | 19237 | CB | ILE | F | 153 | 48.695 | 25.050 | −3.273 | 1.00 | 70.45 | F |
| ATOM | 19238 | CG2 | ILE | F | 153 | 47.214 | 25.175 | −3.561 | 1.00 | 67.23 | F |
| ATOM | 19239 | CG1 | ILE | F | 153 | 49.419 | 24.365 | −4.428 | 1.00 | 67.23 | F |
| ATOM | 19240 | CD1 | ILE | F | 153 | 48.878 | 22.990 | −4.740 | 1.00 | 67.23 | F |
| ATOM | 19241 | C | ILE | F | 153 | 50.671 | 26.274 | −2.400 | 1.00 | 80.49 | F |
| ATOM | 19242 | O | ILE | F | 153 | 51.703 | 26.423 | −3.066 | 1.00 | 80.49 | F |
| ATOM | 19243 | N | THR | F | 154 | 50.667 | 25.944 | −1.112 | 1.00 | 72.49 | F |
| ATOM | 19244 | CA | THR | F | 154 | 51.892 | 25.795 | −0.351 | 1.00 | 72.49 | F |
| ATOM | 19245 | CB | THR | F | 154 | 52.194 | 27.118 | 0.367 | 1.00 | 61.01 | F |
| ATOM | 19246 | OG1 | THR | F | 154 | 53.216 | 27.816 | −0.351 | 1.00 | 71.29 | F |
| ATOM | 19247 | CG2 | THR | F | 154 | 52.612 | 26.884 | 1.830 | 1.00 | 71.29 | F |
| ATOM | 19248 | C | THR | F | 154 | 51.876 | 24.669 | 0.677 | 1.00 | 72.49 | F |
| ATOM | 19249 | O | THR | F | 154 | 50.842 | 24.380 | 1.277 | 1.00 | 72.49 | F |
| ATOM | 19250 | N | LEU | F | 155 | 53.033 | 24.035 | 0.862 | 1.00 | 56.54 | F |
| ATOM | 19251 | CA | LEU | F | 155 | 53.196 | 22.975 | 1.859 | 1.00 | 56.54 | F |
| ATOM | 19252 | CB | LEU | F | 155 | 53.261 | 21.591 | 1.211 | 1.00 | 68.59 | F |
| ATOM | 19253 | CG | LEU | F | 155 | 53.319 | 20.447 | 2.230 | 1.00 | 65.17 | F |
| ATOM | 19254 | CD1 | LEU | F | 155 | 52.149 | 20.557 | 3.192 | 1.00 | 65.17 | F |
| ATOM | 19255 | CD2 | LEU | F | 155 | 53.288 | 19.111 | 1.521 | 1.00 | 65.17 | F |
| ATOM | 19256 | C | LEU | F | 155 | 54.502 | 23.273 | 2.598 | 1.00 | 56.54 | F |
| ATOM | 19257 | O | LEU | F | 155 | 55.588 | 23.273 | 2.002 | 1.00 | 56.54 | F |
| ATOM | 19258 | N | LYS | F | 156 | 54.393 | 23.552 | 3.894 | 1.00 | 67.00 | F |
| ATOM | 19259 | CA | LYS | F | 156 | 55.568 | 23.882 | 4.687 | 1.00 | 67.00 | F |
| ATOM | 19260 | CB | LYS | F | 156 | 55.534 | 25.362 | 5.087 | 1.00 | 78.08 | F |
| ATOM | 19261 | CG | LYS | F | 156 | 56.795 | 25.820 | 5.811 | 1.00 | 80.27 | F |
| ATOM | 19262 | CD | LYS | F | 156 | 56.762 | 27.297 | 6.196 | 1.00 | 80.27 | F |
| ATOM | 19263 | CE | LYS | F | 156 | 55.761 | 27.581 | 7.315 | 1.00 | 80.27 | F |
| ATOM | 19264 | NZ | LYS | F | 156 | 55.844 | 28.991 | 7.807 | 1.00 | 80.27 | F |
| ATOM | 19265 | C | LYS | F | 156 | 55.726 | 23.034 | 5.938 | 1.00 | 67.00 | F |
| ATOM | 19266 | O | LYS | F | 156 | 54.771 | 22.836 | 6.683 | 1.00 | 67.00 | F |
| ATOM | 19267 | N | TYR | F | 157 | 56.944 | 22.534 | 6.148 | 1.00 | 62.06 | F |
| ATOM | 19268 | CA | TYR | F | 157 | 57.301 | 21.725 | 7.316 | 1.00 | 62.06 | F |
| ATOM | 19269 | CB | TYR | F | 157 | 57.919 | 20.387 | 6.886 | 1.00 | 72.13 | F |
| ATOM | 19270 | CG | TYR | F | 157 | 56.924 | 19.366 | 6.385 | 1.00 | 54.47 | F |
| ATOM | 19271 | CD1 | TYR | F | 157 | 55.581 | 19.699 | 6.196 | 1.00 | 54.47 | F |
| ATOM | 19272 | CE1 | TYR | F | 157 | 54.658 | 18.761 | 5.711 | 1.00 | 54.47 | F |
| ATOM | 19273 | CD2 | TYR | F | 157 | 57.328 | 18.069 | 6.084 | 1.00 | 54.47 | F |
| ATOM | 19274 | CE2 | TYR | F | 157 | 56.418 | 17.120 | 5.604 | 1.00 | 54.47 | F |
| ATOM | 19275 | CZ | TYR | F | 157 | 55.083 | 17.472 | 5.413 | 1.00 | 54.47 | F |
| ATOM | 19276 | OH | TYR | F | 157 | 54.184 | 16.554 | 4.904 | 1.00 | 54.47 | F |
| ATOM | 19277 | C | TYR | F | 157 | 58.320 | 22.524 | 8.136 | 1.00 | 62.06 | F |
| ATOM | 19278 | O | TYR | F | 157 | 59.335 | 22.990 | 7.603 | 1.00 | 62.06 | F |
| ATOM | 19279 | N | ASN | F | 158 | 58.044 | 22.679 | 9.429 | 1.00 | 76.98 | F |
| ATOM | 19280 | CA | ASN | F | 158 | 58.917 | 23.442 | 10.319 | 1.00 | 76.98 | F |
| ATOM | 19281 | CB | ASN | F | 158 | 58.208 | 24.744 | 10.729 | 1.00 | 75.34 | F |
| ATOM | 19282 | CG | ASN | F | 158 | 59.072 | 25.656 | 11.594 | 1.00 | 86.04 | F |
| ATOM | 19283 | OD1 | ASN | F | 158 | 60.195 | 25.305 | 11.966 | 1.00 | 86.04 | F |
| ATOM | 19284 | ND2 | ASN | F | 158 | 58.533 | 26.835 | 11.911 | 1.00 | 86.04 | F |
| ATOM | 19285 | C | ASN | F | 158 | 59.262 | 22.626 | 11.557 | 1.00 | 76.98 | F |
| ATOM | 19286 | O | ASN | F | 158 | 58.382 | 22.292 | 12.348 | 1.00 | 76.98 | F |
| ATOM | 19287 | N | TYR | F | 159 | 60.539 | 22.285 | 11.703 | 1.00 | 59.88 | F |
| ATOM | 19288 | CA | TYR | F | 159 | 61.012 | 21.539 | 12.871 | 1.00 | 59.88 | F |
| ATOM | 19289 | CB | TYR | F | 159 | 61.906 | 20.350 | 12.487 | 1.00 | 61.69 | F |
| ATOM | 19290 | CG | TYR | F | 159 | 61.251 | 19.255 | 11.677 | 1.00 | 65.25 | F |
| ATOM | 19291 | CD1 | TYR | F | 159 | 61.120 | 19.370 | 10.293 | 1.00 | 65.25 | F |
| ATOM | 19292 | CE1 | TYR | F | 159 | 60.584 | 18.333 | 9.532 | 1.00 | 65.25 | F |
| ATOM | 19293 | CD2 | TYR | F | 159 | 60.819 | 18.075 | 12.284 | 1.00 | 65.25 | F |
| ATOM | 19294 | CE2 | TYR | F | 159 | 60.282 | 17.031 | 11.534 | 1.00 | 65.25 | F |
| ATOM | 19295 | CZ | TYR | F | 159 | 60.170 | 17.162 | 10.159 | 1.00 | 65.25 | F |
| ATOM | 19296 | OH | TYR | F | 159 | 59.675 | 16.113 | 9.413 | 1.00 | 65.25 | F |
| ATOM | 19297 | C | TYR | F | 159 | 61.855 | 22.509 | 13.687 | 1.00 | 59.88 | F |
| ATOM | 19298 | O | TYR | F | 159 | 62.773 | 23.141 | 13.158 | 1.00 | 59.88 | F |
| ATOM | 19299 | N | THR | F | 160 | 61.562 | 22.622 | 14.977 | 1.00 | 82.88 | F |
| ATOM | 19300 | CA | THR | F | 160 | 62.324 | 23.539 | 15.814 | 1.00 | 82.88 | F |
| ATOM | 19301 | CB | THR | F | 160 | 61.589 | 24.879 | 15.940 | 1.00 | 94.68 | F |
| ATOM | 19302 | OG1 | THR | F | 160 | 61.281 | 25.374 | 14.633 | 1.00 | 82.02 | F |
| ATOM | 19303 | CG2 | THR | F | 160 | 62.453 | 25.892 | 16.663 | 1.00 | 82.02 | F |
| ATOM | 19304 | C | THR | F | 160 | 62.645 | 23.052 | 17.230 | 1.00 | 82.88 | F |
| ATOM | 19305 | O | THR | F | 160 | 61.747 | 22.643 | 17.982 | 1.00 | 82.88 | F |
| ATOM | 19306 | N | VAL | F | 161 | 63.931 | 23.080 | 17.582 | 1.00 | 72.87 | F |
| ATOM | 19307 | CA | VAL | F | 161 | 64.337 | 22.711 | 18.936 | 1.00 | 72.87 | F |
| ATOM | 19308 | CB | VAL | F | 161 | 65.431 | 21.607 | 18.981 | 1.00 | 94.02 | F |
| ATOM | 19309 | CG1 | VAL | F | 161 | 65.008 | 20.422 | 18.142 | 1.00 | 78.53 | F |
| ATOM | 19310 | CG2 | VAL | F | 161 | 66.759 | 22.158 | 18.535 | 1.00 | 78.53 | F |
| ATOM | 19311 | C | VAL | F | 161 | 64.880 | 23.993 | 19.562 | 1.00 | 72.87 | F |
| ATOM | 19312 | O | VAL | F | 161 | 65.626 | 24.736 | 18.926 | 1.00 | 72.87 | F |
| ATOM | 19313 | N | THR | F | 162 | 64.485 | 24.256 | 20.802 | 1.00 | 131.07 | F |
| ATOM | 19314 | CA | THR | F | 162 | 64.908 | 25.466 | 21.493 | 1.00 | 131.07 | F |
| ATOM | 19315 | CB | THR | F | 162 | 63.701 | 26.384 | 21.757 | 1.00 | 99.74 | F |
| ATOM | 19316 | OG1 | THR | F | 162 | 62.765 | 26.270 | 20.678 | 1.00 | 71.07 | F |
| ATOM | 19317 | CG2 | THR | F | 162 | 64.150 | 27.829 | 21.864 | 1.00 | 71.07 | F |
| ATOM | 19318 | C | THR | F | 162 | 65.552 | 25.141 | 22.832 | 1.00 | 131.07 | F |
| ATOM | 19319 | O | THR | F | 162 | 65.201 | 24.148 | 23.471 | 1.00 | 131.07 | F |
| ATOM | 19320 | N | LEU | F | 163 | 66.492 | 25.978 | 23.258 | 1.00 | 104.83 | F |
| ATOM | 19321 | CA | LEU | F | 163 | 67.146 | 25.761 | 24.538 | 1.00 | 104.83 | F |
| ATOM | 19322 | CB | LEU | F | 163 | 68.474 | 26.505 | 24.599 | 1.00 | 141.20 | F |
| ATOM | 19323 | CG | LEU | F | 163 | 69.656 | 25.596 | 24.933 | 1.00 | 89.29 | F |
| ATOM | 19324 | CD1 | LEU | F | 163 | 70.891 | 26.450 | 25.172 | 1.00 | 89.29 | F |
| ATOM | 19325 | CD2 | LEU | F | 163 | 69.342 | 24.753 | 26.164 | 1.00 | 89.29 | F |
| ATOM | 19326 | C | LEU | F | 163 | 66.239 | 26.249 | 25.660 | 1.00 | 104.83 | F |
| ATOM | 19327 | O | LEU | F | 163 | 66.523 | 26.034 | 26.842 | 1.00 | 104.83 | F |
| ATOM | 19328 | N | LYS | F | 164 | 65.139 | 26.897 | 25.282 | 1.00 | 175.79 | F |
| ATOM | 19329 | CA | LYS | F | 164 | 64.192 | 27.419 | 26.259 | 1.00 | 175.79 | F |
| ATOM | 19330 | CB | LYS | F | 164 | 63.385 | 26.275 | 26.881 | 1.00 | 105.07 | F |
| ATOM | 19331 | CG | LYS | F | 164 | 61.976 | 26.145 | 26.329 | 1.00 | 104.82 | F |
| ATOM | 19332 | CD | LYS | F | 164 | 61.158 | 27.402 | 26.624 | 1.00 | 104.82 | F |
| ATOM | 19333 | CE | LYS | F | 164 | 59.728 | 27.296 | 26.109 | 1.00 | 104.82 | F |
| ATOM | 19334 | NZ | LYS | F | 164 | 58.907 | 28.474 | 26.514 | 1.00 | 104.82 | F |
| ATOM | 19335 | C | LYS | F | 164 | 64.970 | 28.151 | 27.338 | 1.00 | 175.79 | F |
| ATOM | 19336 | O | LYS | F | 164 | 66.118 | 28.537 | 27.119 | 1.00 | 175.79 | F |
| ATOM | 19337 | N | ASP | F | 165 | 64.353 | 28.354 | 28.497 | 1.00 | 190.41 | F |
| ATOM | 19338 | CA | ASP | F | 165 | 65.043 | 29.022 | 29.591 | 1.00 | 190.41 | F |
| ATOM | 19339 | CB | ASP | F | 165 | 64.099 | 29.222 | 30.788 | 1.00 | 158.84 | F |
| ATOM | 19340 | CG | ASP | F | 165 | 63.345 | 27.954 | 31.169 | 1.00 | 158.84 | F |
| ATOM | 19341 | OD1 | ASP | F | 165 | 63.989 | 26.969 | 31.592 | 1.00 | 158.84 | F |
| ATOM | 19342 | OD2 | ASP | F | 165 | 62.100 | 27.944 | 31.048 | 1.00 | 158.84 | F |
| ATOM | 19343 | C | ASP | F | 165 | 66.226 | 28.139 | 29.971 | 1.00 | 190.41 | F |
| ATOM | 19344 | O | ASP | F | 165 | 66.958 | 27.664 | 29.102 | 1.00 | 190.41 | F |
| ATOM | 19345 | N | ASP | F | 166 | 66.423 | 27.900 | 31.257 | 1.00 | 128.56 | F |
| ATOM | 19346 | CA | ASP | F | 166 | 67.538 | 27.061 | 31.653 | 1.00 | 128.56 | F |
| ATOM | 19347 | CB | ASP | F | 166 | 68.153 | 27.591 | 32.946 | 1.00 | 183.14 | F |
| ATOM | 19348 | CG | ASP | F | 166 | 68.786 | 28.955 | 32.761 | 1.00 | 183.14 | F |
| ATOM | 19349 | OD1 | ASP | F | 166 | 68.060 | 29.904 | 32.394 | 1.00 | 183.14 | F |
| ATOM | 19350 | OD2 | ASP | F | 166 | 70.010 | 29.077 | 32.973 | 1.00 | 183.14 | F |
| ATOM | 19351 | C | ASP | F | 166 | 67.165 | 25.592 | 31.798 | 1.00 | 128.56 | F |
| ATOM | 19352 | O | ASP | F | 166 | 66.289 | 25.230 | 32.592 | 1.00 | 128.56 | F |
| ATOM | 19353 | N | GLY | F | 167 | 67.821 | 24.752 | 31.001 | 1.00 | 132.34 | F |
| ATOM | 19354 | CA | GLY | F | 167 | 67.565 | 23.330 | 31.083 | 1.00 | 132.34 | F |
| ATOM | 19355 | C | GLY | F | 167 | 67.517 | 22.493 | 29.821 | 1.00 | 132.34 | F |
| ATOM | 19356 | O | GLY | F | 167 | 68.481 | 22.387 | 29.057 | 1.00 | 132.34 | F |
| ATOM | 19357 | N | LEU | F | 168 | 66.356 | 21.885 | 29.633 | 1.00 | 158.15 | F |
| ATOM | 19358 | CA | LEU | F | 168 | 66.082 | 20.995 | 28.523 | 1.00 | 158.15 | F |
| ATOM | 19359 | CB | LEU | F | 168 | 64.808 | 20.210 | 28.842 | 1.00 | 135.04 | F |
| ATOM | 19360 | CG | LEU | F | 168 | 64.589 | 19.996 | 30.346 | 1.00 | 135.04 | F |
| ATOM | 19361 | CD1 | LEU | F | 168 | 63.235 | 19.357 | 30.594 | 1.00 | 135.04 | F |
| ATOM | 19362 | CD2 | LEU | F | 168 | 65.710 | 19.135 | 30.909 | 1.00 | 135.04 | F |
| ATOM | 19363 | C | LEU | F | 168 | 65.931 | 21.704 | 27.183 | 1.00 | 158.15 | F |
| ATOM | 19364 | O | LEU | F | 168 | 65.919 | 22.933 | 27.102 | 1.00 | 158.15 | F |
| ATOM | 19365 | N | TYR | F | 169 | 65.830 | 20.902 | 26.131 | 1.00 | 99.39 | F |
| ATOM | 19366 | CA | TYR | F | 169 | 65.648 | 21.413 | 24.787 | 1.00 | 99.39 | F |
| ATOM | 19367 | CB | TYR | F | 169 | 66.636 | 20.761 | 23.825 | 1.00 | 96.43 | F |
| ATOM | 19368 | CG | TYR | F | 169 | 68.068 | 21.168 | 24.032 | 1.00 | 96.43 | F |
| ATOM | 19369 | CD1 | TYR | F | 169 | 68.821 | 20.638 | 25.072 | 1.00 | 96.43 | F |
| ATOM | 19370 | CE1 | TYR | F | 169 | 70.149 | 21.011 | 25.254 | 1.00 | 96.43 | F |
| ATOM | 19371 | CD2 | TYR | F | 169 | 68.672 | 22.084 | 23.176 | 1.00 | 96.43 | F |
| ATOM | 19372 | CE2 | TYR | F | 169 | 69.993 | 22.466 | 23.345 | 1.00 | 96.43 | F |
| ATOM | 19373 | CZ | TYR | F | 169 | 70.729 | 21.927 | 24.385 | 1.00 | 96.43 | F |
| ATOM | 19374 | OH | TYR | F | 169 | 72.041 | 22.311 | 24.546 | 1.00 | 96.43 | F |
| ATOM | 19375 | C | TYR | F | 169 | 64.235 | 21.055 | 24.360 | 1.00 | 99.39 | F |
| ATOM | 19376 | O | TYR | F | 169 | 63.867 | 19.881 | 24.368 | 1.00 | 99.39 | F |
| ATOM | 19377 | N | ASP | F | 170 | 63.436 | 22.057 | 24.010 | 1.00 | 82.73 | F |
| ATOM | 19378 | CA | ASP | F | 170 | 62.079 | 21.784 | 23.562 | 1.00 | 82.73 | F |
| ATOM | 19379 | CB | ASP | F | 170 | 61.149 | 22.950 | 23.880 | 1.00 | 125.09 | F |
| ATOM | 19380 | CG | ASP | F | 170 | 60.446 | 22.779 | 25.205 | 1.00 | 125.09 | F |
| ATOM | 19381 | OD1 | ASP | F | 170 | 60.075 | 21.632 | 25.533 | 1.00 | 125.09 | F |
| ATOM | 19382 | OD2 | ASP | F | 170 | 60.251 | 23.789 | 25.911 | 1.00 | 125.09 | F |
| ATOM | 19383 | C | ASP | F | 170 | 62.066 | 21.520 | 22.066 | 1.00 | 82.73 | F |
| ATOM | 19384 | O | ASP | F | 170 | 62.946 | 21.974 | 21.339 | 1.00 | 82.73 | F |
| ATOM | 19385 | N | GLY | F | 171 | 61.071 | 20.773 | 21.611 | 1.00 | 104.23 | F |
| ATOM | 19386 | CA | GLY | F | 171 | 60.980 | 20.483 | 20.198 | 1.00 | 104.23 | F |
| ATOM | 19387 | C | GLY | F | 171 | 59.554 | 20.555 | 19.704 | 1.00 | 104.23 | F |
| ATOM | 19388 | O | GLY | F | 171 | 58.638 | 20.044 | 20.353 | 1.00 | 104.23 | F |
| ATOM | 19389 | N | VAL | F | 172 | 59.362 | 21.223 | 18.570 | 1.00 | 63.93 | F |
| ATOM | 19390 | CA | VAL | F | 172 | 58.044 | 21.326 | 17.958 | 1.00 | 63.93 | F |
| ATOM | 19391 | CB | VAL | F | 172 | 57.372 | 22.688 | 18.162 | 1.00 | 66.39 | F |
| ATOM | 19392 | CG1 | VAL | F | 172 | 56.644 | 22.706 | 19.467 | 1.00 | 58.40 | F |
| ATOM | 19393 | CG2 | VAL | F | 172 | 58.408 | 23.798 | 18.074 | 1.00 | 58.40 | F |
| ATOM | 19394 | C | VAL | F | 172 | 58.153 | 21.135 | 16.465 | 1.00 | 63.93 | F |
| ATOM | 19395 | O | VAL | F | 172 | 59.207 | 21.354 | 15.860 | 1.00 | 63.93 | F |
| ATOM | 19396 | N | PHE | F | 173 | 57.035 | 20.736 | 15.880 | 1.00 | 89.37 | F |
| ATOM | 19397 | CA | PHE | F | 173 | 56.946 | 20.528 | 14.457 | 1.00 | 89.37 | F |
| ATOM | 19398 | CB | PHE | F | 173 | 57.054 | 19.049 | 14.121 | 1.00 | 96.81 | F |
| ATOM | 19399 | CG | PHE | F | 173 | 56.668 | 18.746 | 12.718 | 1.00 | 78.63 | F |
| ATOM | 19400 | CD1 | PHE | F | 173 | 57.532 | 19.047 | 11.674 | 1.00 | 78.63 | F |
| ATOM | 19401 | CD2 | PHE | F | 173 | 55.404 | 18.249 | 12.427 | 1.00 | 78.63 | F |
| ATOM | 19402 | CE1 | PHE | F | 173 | 57.142 | 18.864 | 10.365 | 1.00 | 78.63 | F |
| ATOM | 19403 | CE2 | PHE | F | 173 | 55.002 | 18.064 | 11.117 | 1.00 | 78.63 | F |
| ATOM | 19404 | CZ | PHE | F | 173 | 55.871 | 18.373 | 10.083 | 1.00 | 78.63 | F |
| ATOM | 19405 | C | PHE | F | 173 | 55.597 | 21.038 | 13.981 | 1.00 | 89.37 | F |
| ATOM | 19406 | O | PHE | F | 173 | 54.570 | 20.790 | 14.620 | 1.00 | 89.37 | F |
| ATOM | 19407 | N | TYR | F | 174 | 55.598 | 21.754 | 12.863 | 1.00 | 71.23 | F |
| ATOM | 19408 | CA | TYR | F | 174 | 54.355 | 22.264 | 12.303 | 1.00 | 71.23 | F |
| ATOM | 19409 | CB | TYR | F | 174 | 54.268 | 23.788 | 12.414 | 1.00 | 121.19 | F |
| ATOM | 19410 | CG | TYR | F | 174 | 54.151 | 24.308 | 13.822 | 1.00 | 107.23 | F |
| ATOM | 19411 | CD1 | TYR | F | 174 | 55.236 | 24.919 | 14.447 | 1.00 | 107.23 | F |
| ATOM | 19412 | CE1 | TYR | F | 174 | 55.139 | 25.402 | 15.752 | 1.00 | 107.23 | F |
| ATOM | 19413 | CD2 | TYR | F | 174 | 52.955 | 24.187 | 14.537 | 1.00 | 107.23 | F |
| ATOM | 19414 | CE2 | TYR | F | 174 | 52.844 | 24.666 | 15.846 | 1.00 | 107.23 | F |
| ATOM | 19415 | CZ | TYR | F | 174 | 53.943 | 25.273 | 16.446 | 1.00 | 107.23 | F |
| ATOM | 19416 | OH | TYR | F | 174 | 53.851 | 25.757 | 17.733 | 1.00 | 107.23 | F |
| ATOM | 19417 | C | TYR | F | 174 | 54.259 | 21.884 | 10.841 | 1.00 | 71.23 | F |
| ATOM | 19418 | O | TYR | F | 174 | 55.247 | 21.973 | 10.107 | 1.00 | 71.23 | F |
| ATOM | 19419 | N | ASP | F | 175 | 53.061 | 21.454 | 10.442 | 1.00 | 74.88 | F |
| ATOM | 19420 | CA | ASP | F | 175 | 52.755 | 21.077 | 9.066 | 1.00 | 74.88 | F |
| ATOM | 19421 | CB | ASP | F | 175 | 52.184 | 19.655 | 9.010 | 1.00 | 116.75 | F |
| ATOM | 19422 | CG | ASP | F | 175 | 51.964 | 19.162 | 7.584 | 1.00 | 104.96 | F |
| ATOM | 19423 | OD1 | ASP | F | 175 | 51.671 | 19.988 | 6.697 | 1.00 | 104.96 | F |
| ATOM | 19424 | OD2 | ASP | F | 175 | 52.065 | 17.942 | 7.348 | 1.00 | 104.96 | F |
| ATOM | 19425 | C | ASP | F | 175 | 51.694 | 22.079 | 8.609 | 1.00 | 74.88 | F |
| ATOM | 19426 | O | ASP | F | 175 | 50.574 | 22.087 | 9.125 | 1.00 | 74.88 | F |
| ATOM | 19427 | N | HIS | F | 176 | 52.050 | 22.928 | 7.652 | 1.00 | 84.68 | F |
| ATOM | 19428 | CA | HIS | F | 176 | 51.122 | 23.931 | 7.145 | 1.00 | 84.68 | F |
| ATOM | 19429 | CB | HIS | F | 176 | 51.669 | 25.342 | 7.394 | 1.00 | 113.68 | F |
| ATOM | 19430 | CG | HIS | F | 176 | 51.570 | 25.801 | 8.816 | 1.00 | 82.81 | F |
| ATOM | 19431 | CD2 | HIS | F | 176 | 52.528 | 26.020 | 9.748 | 1.00 | 82.81 | F |
| ATOM | 19432 | ND1 | HIS | F | 176 | 50.372 | 26.133 | 9.411 | 1.00 | 82.81 | F |
| ATOM | 19433 | CE1 | HIS | F | 176 | 50.596 | 26.540 | 10.648 | 1.00 | 82.81 | F |
| ATOM | 19434 | NE2 | HIS | F | 176 | 51.896 | 26.481 | 10.876 | 1.00 | 82.81 | F |
| ATOM | 19435 | C | HIS | F | 176 | 50.834 | 23.805 | 5.653 | 1.00 | 84.68 | F |
| ATOM | 19436 | O | HIS | F | 176 | 51.753 | 23.791 | 4.833 | 1.00 | 84.68 | F |
| ATOM | 19437 | N | TYR | F | 177 | 49.553 | 23.703 | 5.312 | 1.00 | 85.03 | F |
| ATOM | 19438 | CA | TYR | F | 177 | 49.126 | 23.663 | 3.919 | 1.00 | 85.03 | F |
| ATOM | 19439 | CB | TYR | F | 177 | 48.016 | 22.643 | 3.705 | 1.00 | 99.56 | F |
| ATOM | 19440 | CG | TYR | F | 177 | 47.509 | 22.667 | 2.288 | 1.00 | 92.57 | F |
| ATOM | 19441 | CD1 | TYR | F | 177 | 48.320 | 22.253 | 1.237 | 1.00 | 92.57 | F |
| ATOM | 19442 | CE1 | TYR | F | 177 | 47.886 | 22.329 | −0.080 | 1.00 | 92.57 | F |
| ATOM | 19443 | CD2 | TYR | F | 177 | 46.242 | 23.158 | 1.987 | 1.00 | 92.57 | F |
| ATOM | 19444 | CE2 | TYR | F | 177 | 45.797 | 23.241 | 0.672 | 1.00 | 92.57 | F |
| ATOM | 19445 | CZ | TYR | F | 177 | 46.624 | 22.824 | −0.357 | 1.00 | 92.57 | F |
| ATOM | 19446 | OH | TYR | F | 177 | 46.192 | 22.907 | −1.663 | 1.00 | 92.57 | F |
| ATOM | 19447 | C | TYR | F | 177 | 48.566 | 25.071 | 3.731 | 1.00 | 85.03 | F |
| ATOM | 19448 | O | TYR | F | 177 | 47.563 | 25.419 | 4.352 | 1.00 | 85.03 | F |
| ATOM | 19449 | N | ASN | F | 178 | 49.193 | 25.881 | 2.880 | 1.00 | 99.21 | F |
| ATOM | 19450 | CA | ASN | F | 178 | 48.756 | 27.269 | 2.717 | 1.00 | 99.21 | F |
| ATOM | 19451 | CB | ASN | F | 178 | 47.289 | 27.367 | 2.275 | 1.00 | 97.73 | F |
| ATOM | 19452 | CG | ASN | F | 178 | 47.093 | 27.015 | 0.816 | 1.00 | 90.20 | F |
| ATOM | 19453 | OD1 | ASN | F | 178 | 48.057 | 26.907 | 0.061 | 1.00 | 90.20 | F |
| ATOM | 19454 | ND2 | ASN | F | 178 | 45.838 | 26.849 | 0.407 | 1.00 | 90.20 | F |
| ATOM | 19455 | C | ASN | F | 178 | 48.907 | 27.835 | 4.123 | 1.00 | 99.21 | F |
| ATOM | 19456 | O | ASN | F | 178 | 49.921 | 27.599 | 4.777 | 1.00 | 99.21 | F |
| ATOM | 19457 | N | ASP | F | 179 | 47.907 | 28.560 | 4.604 | 1.00 | 101.68 | F |
| ATOM | 19458 | CA | ASP | F | 179 | 47.988 | 29.101 | 5.951 | 1.00 | 101.68 | F |
| ATOM | 19459 | CB | ASP | F | 179 | 47.458 | 30.546 | 5.990 | 1.00 | 74.50 | F |
| ATOM | 19460 | CG | ASP | F | 179 | 46.037 | 30.678 | 5.447 | 1.00 | 78.45 | F |
| ATOM | 19461 | OD1 | ASP | F | 179 | 45.415 | 29.658 | 5.074 | 1.00 | 78.45 | F |
| ATOM | 19462 | OD2 | ASP | F | 179 | 45.537 | 31.819 | 5.396 | 1.00 | 78.45 | F |
| ATOM | 19463 | C | ASP | F | 179 | 47.227 | 28.221 | 6.947 | 1.00 | 101.68 | F |
| ATOM | 19464 | O | ASP | F | 179 | 46.973 | 28.634 | 8.076 | 1.00 | 101.68 | F |
| ATOM | 19465 | N | GLN | F | 180 | 46.887 | 27.002 | 6.531 | 1.00 | 89.25 | F |
| ATOM | 19466 | CA | GLN | F | 180 | 46.148 | 26.060 | 7.378 | 1.00 | 89.25 | F |
| ATOM | 19467 | CB | GLN | F | 180 | 45.182 | 25.233 | 6.531 | 1.00 | 88.39 | F |
| ATOM | 19468 | CG | GLN | F | 180 | 44.237 | 26.035 | 5.689 | 1.00 | 80.02 | F |
| ATOM | 19469 | CD | GLN | F | 180 | 43.392 | 26.958 | 6.515 | 1.00 | 80.02 | F |
| ATOM | 19470 | OE1 | GLN | F | 180 | 43.667 | 28.156 | 6.606 | 1.00 | 80.02 | F |
| ATOM | 19471 | NE2 | GLN | F | 180 | 42.357 | 26.405 | 7.141 | 1.00 | 80.02 | F |
| ATOM | 19472 | C | GLN | F | 180 | 47.021 | 25.078 | 8.162 | 1.00 | 89.25 | F |
| ATOM | 19473 | O | GLN | F | 180 | 47.752 | 24.278 | 7.576 | 1.00 | 89.25 | F |
| ATOM | 19474 | N | LEU | F | 181 | 46.931 | 25.116 | 9.487 | 1.00 | 79.33 | F |
| ATOM | 19475 | CA | LEU | F | 181 | 47.700 | 24.183 | 10.297 | 1.00 | 79.33 | F |
| ATOM | 19476 | CB | LEU | F | 181 | 47.588 | 24.539 | 11.779 | 1.00 | 82.02 | F |
| ATOM | 19477 | CG | LEU | F | 181 | 48.262 | 23.535 | 12.713 | 1.00 | 69.45 | F |
| ATOM | 19478 | CD1 | LEU | F | 181 | 49.756 | 23.477 | 12.406 | 1.00 | 69.45 | F |
| ATOM | 19479 | CD2 | LEU | F | 181 | 48.013 | 23.932 | 14.160 | 1.00 | 69.45 | F |
| ATOM | 19480 | C | LEU | F | 181 | 47.133 | 22.782 | 10.054 | 1.00 | 79.33 | F |
| ATOM | 19481 | O | LEU | F | 181 | 45.918 | 22.573 | 10.104 | 1.00 | 79.33 | F |
| ATOM | 19482 | N | VAL | F | 182 | 48.010 | 21.826 | 9.777 | 1.00 | 95.52 | F |
| ATOM | 19483 | CA | VAL | F | 182 | 47.572 | 20.461 | 9.525 | 1.00 | 95.52 | F |
| ATOM | 19484 | CB | VAL | F | 182 | 48.175 | 19.926 | 8.218 | 1.00 | 119.01 | F |
| ATOM | 19485 | CG1 | VAL | F | 182 | 47.750 | 18.485 | 7.997 | 1.00 | 97.49 | F |
| ATOM | 19486 | CG2 | VAL | F | 182 | 47.736 | 20.796 | 7.061 | 1.00 | 97.49 | F |
| ATOM | 19487 | C | VAL | F | 182 | 47.980 | 19.532 | 10.660 | 1.00 | 95.52 | F |
| ATOM | 19488 | O | VAL | F | 182 | 47.215 | 18.652 | 11.064 | 1.00 | 95.52 | F |
| ATOM | 19489 | N | THR | F | 183 | 49.188 | 19.742 | 11.175 | 1.00 | 63.20 | F |
| ATOM | 19490 | CA | THR | F | 183 | 49.715 | 18.911 | 12.245 | 1.00 | 63.20 | F |
| ATOM | 19491 | CB | THR | F | 183 | 50.483 | 17.694 | 11.682 | 1.00 | 103.18 | F |
| ATOM | 19492 | OG1 | THR | F | 183 | 49.576 | 16.833 | 10.986 | 1.00 | 89.77 | F |
| ATOM | 19493 | CG2 | THR | F | 183 | 51.162 | 16.917 | 12.800 | 1.00 | 89.77 | F |
| ATOM | 19494 | C | THR | F | 183 | 50.683 | 19.653 | 13.144 | 1.00 | 63.20 | F |
| ATOM | 19495 | O | THR | F | 183 | 51.504 | 20.451 | 12.676 | 1.00 | 63.20 | F |
| ATOM | 19496 | N | LYS | F | 184 | 50.572 | 19.392 | 14.441 | 1.00 | 84.94 | F |
| ATOM | 19497 | CA | LYS | F | 184 | 51.489 | 19.971 | 15.403 | 1.00 | 84.94 | F |
| ATOM | 19498 | CB | LYS | F | 184 | 50.820 | 20.988 | 16.319 | 1.00 | 99.71 | F |
| ATOM | 19499 | CG | LYS | F | 184 | 51.810 | 21.534 | 17.338 | 1.00 | 91.55 | F |
| ATOM | 19500 | CD | LYS | F | 184 | 51.171 | 22.471 | 18.338 | 1.00 | 91.55 | F |
| ATOM | 19501 | CE | LYS | F | 184 | 52.183 | 22.913 | 19.385 | 1.00 | 91.55 | F |
| ATOM | 19502 | NZ | LYS | F | 184 | 51.565 | 23.761 | 20.443 | 1.00 | 91.55 | F |
| ATOM | 19503 | C | LYS | F | 184 | 52.008 | 18.823 | 16.242 | 1.00 | 84.94 | F |
| ATOM | 19504 | O | LYS | F | 184 | 51.245 | 17.967 | 16.694 | 1.00 | 84.94 | F |
| ATOM | 19505 | N | ILE | F | 185 | 53.317 | 18.795 | 16.425 | 1.00 | 65.30 | F |
| ATOM | 19506 | CA | ILE | F | 185 | 53.944 | 17.755 | 17.220 | 1.00 | 65.30 | F |
| ATOM | 19507 | CB | ILE | F | 185 | 54.659 | 16.723 | 16.324 | 1.00 | 115.19 | F |
| ATOM | 19508 | CG2 | ILE | F | 185 | 55.539 | 15.809 | 17.167 | 1.00 | 84.11 | F |
| ATOM | 19509 | CG1 | ILE | F | 185 | 53.620 | 15.903 | 15.558 | 1.00 | 84.11 | F |
| ATOM | 19510 | CD1 | ILE | F | 185 | 54.221 | 14.976 | 14.519 | 1.00 | 84.11 | F |
| ATOM | 19511 | C | ILE | F | 185 | 54.955 | 18.410 | 18.157 | 1.00 | 65.30 | F |
| ATOM | 19512 | O | ILE | F | 185 | 55.654 | 19.358 | 17.779 | 1.00 | 65.30 | F |
| ATOM | 19513 | N | SER | F | 186 | 55.004 | 17.930 | 19.395 | 1.00 | 84.41 | F |
| ATOM | 19514 | CA | SER | F | 186 | 55.948 | 18.467 | 20.362 | 1.00 | 84.41 | F |
| ATOM | 19515 | CB | SER | F | 186 | 55.261 | 19.473 | 21.292 | 1.00 | 132.14 | F |
| ATOM | 19516 | OG | SER | F | 186 | 54.020 | 18.982 | 21.755 | 1.00 | 121.25 | F |
| ATOM | 19517 | C | SER | F | 186 | 56.606 | 17.342 | 21.152 | 1.00 | 84.41 | F |
| ATOM | 19518 | O | SER | F | 186 | 56.036 | 16.256 | 21.329 | 1.00 | 84.41 | F |
| ATOM | 19519 | N | TYR | F | 187 | 57.827 | 17.607 | 21.597 | 1.00 | 86.97 | F |
| ATOM | 19520 | CA | TYR | F | 187 | 58.596 | 16.629 | 22.340 | 1.00 | 86.97 | F |
| ATOM | 19521 | CB | TYR | F | 187 | 60.070 | 17.007 | 22.317 | 1.00 | 134.73 | F |
| ATOM | 19522 | CG | TYR | F | 187 | 60.946 | 15.963 | 22.943 | 1.00 | 88.94 | F |
| ATOM | 19523 | CD1 | TYR | F | 187 | 61.149 | 14.738 | 22.319 | 1.00 | 88.94 | F |
| ATOM | 19524 | CE1 | TYR | F | 187 | 61.950 | 13.760 | 22.900 | 1.00 | 88.94 | F |
| ATOM | 19525 | CD2 | TYR | F | 187 | 61.563 | 16.190 | 24.169 | 1.00 | 88.94 | F |
| ATOM | 19526 | CE2 | TYR | F | 187 | 62.365 | 15.220 | 24.761 | 1.00 | 88.94 | F |
| ATOM | 19527 | CZ | TYR | F | 187 | 62.557 | 14.006 | 24.121 | 1.00 | 88.94 | F |
| ATOM | 19528 | OH | TYR | F | 187 | 63.361 | 13.041 | 24.690 | 1.00 | 88.94 | F |
| ATOM | 19529 | C | TYR | F | 187 | 58.129 | 16.518 | 23.780 | 1.00 | 86.97 | F |
| ATOM | 19530 | O | TYR | F | 187 | 58.149 | 17.498 | 24.524 | 1.00 | 86.97 | F |
| ATOM | 19531 | N | ASN | F | 188 | 57.712 | 15.315 | 24.160 | 1.00 | 140.25 | F |
| ATOM | 19532 | CA | ASN | F | 188 | 57.239 | 15.036 | 25.510 | 1.00 | 140.25 | F |
| ATOM | 19533 | CB | ASN | F | 188 | 56.253 | 13.870 | 25.466 | 1.00 | 200.84 | F |
| ATOM | 19534 | CG | ASN | F | 188 | 55.680 | 13.545 | 26.820 | 1.00 | 118.55 | F |
| ATOM | 19535 | OD1 | ASN | F | 188 | 56.416 | 13.349 | 27.786 | 1.00 | 118.55 | F |
| ATOM | 19536 | ND2 | ASN | F | 188 | 54.358 | 13.479 | 26.901 | 1.00 | 118.55 | F |
| ATOM | 19537 | C | ASN | F | 188 | 58.437 | 14.674 | 26.389 | 1.00 | 140.25 | F |
| ATOM | 19538 | O | ASN | F | 188 | 59.053 | 13.625 | 26.200 | 1.00 | 140.25 | F |
| ATOM | 19539 | N | HIS | F | 189 | 58.763 | 15.533 | 27.351 | 1.00 | 102.72 | F |
| ATOM | 19540 | CA | HIS | F | 189 | 59.907 | 15.291 | 28.228 | 1.00 | 102.72 | F |
| ATOM | 19541 | CB | HIS | F | 189 | 60.222 | 16.557 | 29.004 | 1.00 | 142.88 | F |
| ATOM | 19542 | CG | HIS | F | 189 | 60.841 | 17.615 | 28.157 | 1.00 | 103.42 | F |
| ATOM | 19543 | CD2 | HIS | F | 189 | 60.311 | 18.713 | 27.569 | 1.00 | 103.42 | F |
| ATOM | 19544 | ND1 | HIS | F | 189 | 62.158 | 17.561 | 27.755 | 1.00 | 103.42 | F |
| ATOM | 19545 | CE1 | HIS | F | 189 | 62.413 | 18.581 | 26.954 | 1.00 | 103.42 | F |
| ATOM | 19546 | NE2 | HIS | F | 189 | 61.308 | 19.295 | 26.825 | 1.00 | 103.42 | F |
| ATOM | 19547 | C | HIS | F | 189 | 59.761 | 14.115 | 29.180 | 1.00 | 102.72 | F |
| ATOM | 19548 | O | HIS | F | 189 | 60.751 | 13.494 | 29.574 | 1.00 | 102.72 | F |
| ATOM | 19549 | N | GLU | F | 190 | 58.525 | 13.812 | 29.547 | 1.00 | 127.49 | F |
| ATOM | 19550 | CA | GLU | F | 190 | 58.254 | 12.704 | 30.443 | 1.00 | 127.49 | F |
| ATOM | 19551 | CB | GLU | F | 190 | 56.795 | 12.772 | 30.896 | 1.00 | 189.02 | F |
| ATOM | 19552 | CG | GLU | F | 190 | 56.490 | 12.077 | 32.208 | 1.00 | 163.44 | F |
| ATOM | 19553 | CD | GLU | F | 190 | 55.052 | 12.291 | 32.642 | 1.00 | 163.44 | F |
| ATOM | 19554 | OE1 | GLU | F | 190 | 54.139 | 11.810 | 31.935 | 1.00 | 163.44 | F |
| ATOM | 19555 | OE2 | GLU | F | 190 | 54.833 | 12.947 | 33.683 | 1.00 | 163.44 | F |
| ATOM | 19556 | C | GLU | F | 190 | 58.514 | 11.412 | 29.670 | 1.00 | 127.49 | F |
| ATOM | 19557 | O | GLU | F | 190 | 59.323 | 10.580 | 30.079 | 1.00 | 127.49 | F |
| ATOM | 19558 | N | THR | F | 191 | 57.839 | 11.277 | 28.531 | 1.00 | 124.21 | F |
| ATOM | 19559 | CA | THR | F | 191 | 57.944 | 10.100 | 27.676 | 1.00 | 124.21 | F |
| ATOM | 19560 | CB | THR | F | 191 | 56.751 | 10.033 | 26.713 | 1.00 | 114.85 | F |
| ATOM | 19561 | OG1 | THR | F | 191 | 55.537 | 9.926 | 27.463 | 1.00 | 114.85 | F |
| ATOM | 19562 | CG2 | THR | F | 191 | 56.876 | 8.835 | 25.802 | 1.00 | 114.85 | F |
| ATOM | 19563 | C | THR | F | 191 | 59.224 | 9.996 | 26.850 | 1.00 | 124.21 | F |
| ATOM | 19564 | O | THR | F | 191 | 59.668 | 8.893 | 26.527 | 1.00 | 124.21 | F |
| ATOM | 19565 | N | ARG | F | 192 | 59.800 | 11.139 | 26.495 | 1.00 | 114.81 | F |
| ATOM | 19566 | CA | ARG | F | 192 | 61.028 | 11.179 | 25.703 | 1.00 | 114.81 | F |
| ATOM | 19567 | CB | ARG | F | 192 | 62.066 | 10.208 | 26.274 | 1.00 | 118.10 | F |
| ATOM | 19568 | CG | ARG | F | 192 | 62.561 | 10.604 | 27.645 | 1.00 | 118.10 | F |
| ATOM | 19569 | CD | ARG | F | 192 | 63.153 | 12.001 | 27.611 | 1.00 | 118.10 | F |
| ATOM | 19570 | NE | ARG | F | 192 | 63.459 | 12.500 | 28.949 | 1.00 | 118.10 | F |
| ATOM | 19571 | CZ | ARG | F | 192 | 64.374 | 11.971 | 29.755 | 1.00 | 118.10 | F |
| ATOM | 19572 | NH1 | ARG | F | 192 | 65.084 | 10.922 | 29.359 | 1.00 | 118.10 | F |
| ATOM | 19573 | NH2 | ARG | F | 192 | 64.576 | 12.487 | 30.960 | 1.00 | 118.10 | F |
| ATOM | 19574 | C | ARG | F | 192 | 60.834 | 10.903 | 24.216 | 1.00 | 114.81 | F |
| ATOM | 19575 | O | ARG | F | 192 | 61.728 | 10.372 | 23.559 | 1.00 | 114.81 | F |
| ATOM | 19576 | N | HIS | F | 193 | 59.663 | 11.257 | 23.694 | 1.00 | 116.71 | F |
| ATOM | 19577 | CA | HIS | F | 193 | 59.364 | 11.093 | 22.276 | 1.00 | 116.71 | F |
| ATOM | 19578 | CB | HIS | F | 193 | 58.928 | 9.650 | 21.968 | 1.00 | 130.39 | F |
| ATOM | 19579 | CG | HIS | F | 193 | 57.559 | 9.293 | 22.455 | 1.00 | 130.39 | F |
| ATOM | 19580 | CD2 | HIS | F | 193 | 56.511 | 10.061 | 22.836 | 1.00 | 130.39 | F |
| ATOM | 19581 | ND1 | HIS | F | 193 | 57.124 | 7.988 | 22.532 | 1.00 | 130.39 | F |
| ATOM | 19582 | CE1 | HIS | F | 193 | 55.867 | 7.967 | 22.937 | 1.00 | 130.39 | F |
| ATOM | 19583 | NE2 | HIS | F | 193 | 55.471 | 9.212 | 23.128 | 1.00 | 130.39 | F |
| ATOM | 19584 | C | HIS | F | 193 | 58.283 | 12.110 | 21.915 | 1.00 | 116.71 | F |
| ATOM | 19585 | O | HIS | F | 193 | 57.712 | 12.733 | 22.806 | 1.00 | 116.71 | F |
| ATOM | 19586 | N | GLY | F | 194 | 58.002 | 12.291 | 20.629 | 1.00 | 90.52 | F |
| ATOM | 19587 | CA | GLY | F | 194 | 57.005 | 13.275 | 20.239 | 1.00 | 90.52 | F |
| ATOM | 19588 | C | GLY | F | 194 | 55.559 | 12.845 | 20.387 | 1.00 | 90.52 | F |
| ATOM | 19589 | O | GLY | F | 194 | 55.269 | 11.656 | 20.487 | 1.00 | 90.52 | F |
| ATOM | 19590 | N | ASN | F | 195 | 54.652 | 13.819 | 20.418 | 1.00 | 92.73 | F |
| ATOM | 19591 | CA | ASN | F | 195 | 53.218 | 13.552 | 20.520 | 1.00 | 92.73 | F |
| ATOM | 19592 | CB | ASN | F | 195 | 52.682 | 13.827 | 21.928 | 1.00 | 124.37 | F |
| ATOM | 19593 | CG | ASN | F | 195 | 53.171 | 12.836 | 22.951 | 1.00 | 124.37 | F |
| ATOM | 19594 | OD1 | ASN | F | 195 | 53.171 | 11.628 | 22.718 | 1.00 | 124.37 | F |
| ATOM | 19595 | ND2 | ASN | F | 195 | 53.572 | 13.342 | 24.110 | 1.00 | 124.37 | F |
| ATOM | 19596 | C | ASN | F | 195 | 52.485 | 14.478 | 19.566 | 1.00 | 92.73 | F |
| ATOM | 19597 | O | ASN | F | 195 | 52.846 | 15.648 | 19.439 | 1.00 | 92.73 | F |
| ATOM | 19598 | N | VAL | F | 196 | 51.460 | 13.967 | 18.896 | 1.00 | 118.41 | F |
| ATOM | 19599 | CA | VAL | F | 196 | 50.693 | 14.810 | 17.994 | 1.00 | 118.41 | F |
| ATOM | 19600 | CB | VAL | F | 196 | 49.887 | 13.987 | 16.980 | 1.00 | 101.50 | F |
| ATOM | 19601 | CG1 | VAL | F | 196 | 49.049 | 14.915 | 16.121 | 1.00 | 101.50 | F |
| ATOM | 19602 | CG2 | VAL | F | 196 | 50.823 | 13.171 | 16.113 | 1.00 | 101.50 | F |
| ATOM | 19603 | C | VAL | F | 196 | 49.719 | 15.589 | 18.860 | 1.00 | 118.41 | F |
| ATOM | 19604 | O | VAL | F | 196 | 48.869 | 14.999 | 19.523 | 1.00 | 118.41 | F |
| ATOM | 19605 | N | ASN | F | 197 | 49.851 | 16.910 | 18.868 | 1.00 | 109.92 | F |
| ATOM | 19606 | CA | ASN | F | 197 | 48.969 | 17.750 | 19.672 | 1.00 | 109.92 | F |
| ATOM | 19607 | CB | ASN | F | 197 | 49.699 | 18.996 | 20.164 | 1.00 | 118.36 | F |
| ATOM | 19608 | CG | ASN | F | 197 | 51.046 | 18.685 | 20.747 | 1.00 | 118.36 | F |
| ATOM | 19609 | OD1 | ASN | F | 197 | 51.185 | 17.775 | 21.564 | 1.00 | 118.36 | F |
| ATOM | 19610 | ND2 | ASN | F | 197 | 52.056 | 19.447 | 20.338 | 1.00 | 118.36 | F |
| ATOM | 19611 | C | ASN | F | 197 | 47.774 | 18.199 | 18.857 | 1.00 | 109.92 | F |
| ATOM | 19612 | O | ASN | F | 197 | 46.682 | 18.387 | 19.387 | 1.00 | 109.92 | F |
| ATOM | 19613 | N | PHE | F | 198 | 47.990 | 18.375 | 17.561 | 1.00 | 102.23 | F |
| ATOM | 19614 | CA | PHE | F | 198 | 46.930 | 18.829 | 16.688 | 1.00 | 102.23 | F |
| ATOM | 19615 | CB | PHE | F | 198 | 47.070 | 20.329 | 16.452 | 1.00 | 101.15 | F |
| ATOM | 19616 | CG | PHE | F | 198 | 45.985 | 20.902 | 15.599 | 1.00 | 101.15 | F |
| ATOM | 19617 | CD1 | PHE | F | 198 | 44.824 | 21.403 | 16.172 | 1.00 | 101.15 | F |
| ATOM | 19618 | CD2 | PHE | F | 198 | 46.104 | 20.901 | 14.213 | 1.00 | 101.15 | F |
| ATOM | 19619 | CE1 | PHE | F | 198 | 43.795 | 21.892 | 15.381 | 1.00 | 101.15 | F |
| ATOM | 19620 | CE2 | PHE | F | 198 | 45.078 | 21.388 | 13.410 | 1.00 | 101.15 | F |
| ATOM | 19621 | CZ | PHE | F | 198 | 43.922 | 21.884 | 13.995 | 1.00 | 101.15 | F |
| ATOM | 19622 | C | PHE | F | 198 | 46.925 | 18.126 | 15.344 | 1.00 | 102.23 | F |
| ATOM | 19623 | O | PHE | F | 198 | 47.974 | 17.791 | 14.797 | 1.00 | 102.23 | F |
| ATOM | 19624 | N | ARG | F | 199 | 45.728 | 17.915 | 14.816 | 1.00 | 103.86 | F |
| ATOM | 19625 | CA | ARG | F | 199 | 45.564 | 17.292 | 13.517 | 1.00 | 103.86 | F |
| ATOM | 19626 | CB | ARG | F | 199 | 45.441 | 15.777 | 13.630 | 1.00 | 88.07 | F |
| ATOM | 19627 | CG | ARG | F | 199 | 45.235 | 15.134 | 12.267 | 1.00 | 88.07 | F |
| ATOM | 19628 | CD | ARG | F | 199 | 46.527 | 15.132 | 11.465 | 1.00 | 88.07 | F |
| ATOM | 19629 | NE | ARG | F | 199 | 47.186 | 13.834 | 11.581 | 1.00 | 88.07 | F |
| ATOM | 19630 | CZ | ARG | F | 199 | 48.498 | 13.654 | 11.683 | 1.00 | 88.07 | F |
| ATOM | 19631 | NH1 | ARG | F | 199 | 49.316 | 14.695 | 11.687 | 1.00 | 88.07 | F |
| ATOM | 19632 | NH2 | ARG | F | 199 | 48.993 | 12.426 | 11.787 | 1.00 | 88.07 | F |
| ATOM | 19633 | C | ARG | F | 199 | 44.296 | 17.838 | 12.894 | 1.00 | 103.86 | F |
| ATOM | 19634 | O | ARG | F | 199 | 43.207 | 17.661 | 13.438 | 1.00 | 103.86 | F |
| ATOM | 19635 | N | ALA | F | 200 | 44.436 | 18.508 | 11.758 | 1.00 | 95.58 | F |
| ATOM | 19636 | CA | ALA | F | 200 | 43.281 | 19.064 | 11.073 | 1.00 | 95.58 | F |
| ATOM | 19637 | CB | ALA | F | 200 | 43.718 | 19.741 | 9.786 | 1.00 | 77.98 | F |
| ATOM | 19638 | C | ALA | F | 200 | 42.267 | 17.959 | 10.777 | 1.00 | 95.58 | F |
| ATOM | 19639 | O | ALA | F | 200 | 42.627 | 16.860 | 10.345 | 1.00 | 95.58 | F |
| ATOM | 19640 | N | ASP | F | 201 | 40.998 | 18.261 | 11.021 | 1.00 | 122.99 | F |
| ATOM | 19641 | CA | ASP | F | 201 | 39.908 | 17.318 | 10.802 | 1.00 | 122.99 | F |
| ATOM | 19642 | CB | ASP | F | 201 | 38.592 | 17.957 | 11.247 | 1.00 | 126.05 | F |
| ATOM | 19643 | CG | ASP | F | 201 | 38.403 | 19.348 | 10.670 | 1.00 | 126.05 | F |
| ATOM | 19644 | OD1 | ASP | F | 201 | 39.170 | 20.260 | 11.055 | 1.00 | 126.05 | F |
| ATOM | 19645 | OD2 | ASP | F | 201 | 37.498 | 19.528 | 9.825 | 1.00 | 126.05 | F |
| ATOM | 19646 | C | ASP | F | 201 | 39.769 | 16.838 | 9.355 | 1.00 | 122.99 | F |
| ATOM | 19647 | O | ASP | F | 201 | 39.314 | 15.720 | 9.113 | 1.00 | 122.99 | F |
| ATOM | 19648 | N | TRP | F | 202 | 40.160 | 17.676 | 8.397 | 1.00 | 104.82 | F |
| ATOM | 19649 | CA | TRP | F | 202 | 40.042 | 17.323 | 6.980 | 1.00 | 104.82 | F |
| ATOM | 19650 | CB | TRP | F | 202 | 39.859 | 18.581 | 6.128 | 1.00 | 97.26 | F |
| ATOM | 19651 | CG | TRP | F | 202 | 40.916 | 19.634 | 6.321 | 1.00 | 97.26 | F |
| ATOM | 19652 | CD2 | TRP | F | 202 | 42.160 | 19.745 | 5.618 | 1.00 | 97.26 | F |
| ATOM | 19653 | CE2 | TRP | F | 202 | 42.807 | 20.910 | 6.094 | 1.00 | 97.26 | F |
| ATOM | 19654 | CE3 | TRP | F | 202 | 42.791 | 18.976 | 4.633 | 1.00 | 97.26 | F |
| ATOM | 19655 | CD1 | TRP | F | 202 | 40.864 | 20.700 | 7.173 | 1.00 | 97.26 | F |
| ATOM | 19656 | NE1 | TRP | F | 202 | 41.994 | 21.473 | 7.040 | 1.00 | 97.26 | F |
| ATOM | 19657 | CZ2 | TRP | F | 202 | 44.052 | 21.324 | 5.616 | 1.00 | 97.26 | F |
| ATOM | 19658 | CZ3 | TRP | F | 202 | 44.031 | 19.388 | 4.159 | 1.00 | 97.26 | F |
| ATOM | 19659 | CH2 | TRP | F | 202 | 44.647 | 20.553 | 4.652 | 1.00 | 97.26 | F |
| ATOM | 19660 | C | TRP | F | 202 | 41.188 | 16.518 | 6.384 | 1.00 | 104.82 | F |
| ATOM | 19661 | O | TRP | F | 202 | 41.099 | 16.060 | 5.248 | 1.00 | 104.82 | F |
| ATOM | 19662 | N | PHE | F | 203 | 42.259 | 16.338 | 7.145 | 1.00 | 112.60 | F |
| ATOM | 19663 | CA | PHE | F | 203 | 43.420 | 15.615 | 6.652 | 1.00 | 112.60 | F |
| ATOM | 19664 | CB | PHE | F | 203 | 44.687 | 16.361 | 7.072 | 1.00 | 94.83 | F |
| ATOM | 19665 | CG | PHE | F | 203 | 45.945 | 15.824 | 6.458 | 1.00 | 94.83 | F |
| ATOM | 19666 | CD1 | PHE | F | 203 | 46.193 | 15.984 | 5.098 | 1.00 | 94.83 | F |
| ATOM | 19667 | CD2 | PHE | F | 203 | 46.892 | 15.170 | 7.241 | 1.00 | 94.83 | F |
| ATOM | 19668 | CE1 | PHE | F | 203 | 47.367 | 15.504 | 4.525 | 1.00 | 94.83 | F |
| ATOM | 19669 | CE2 | PHE | F | 203 | 48.068 | 14.686 | 6.680 | 1.00 | 94.83 | F |
| ATOM | 19670 | CZ | PHE | F | 203 | 48.306 | 14.855 | 5.319 | 1.00 | 94.83 | F |
| ATOM | 19671 | C | PHE | F | 203 | 43.505 | 14.172 | 7.130 | 1.00 | 112.60 | F |
| ATOM | 19672 | O | PHE | F | 203 | 43.817 | 13.917 | 8.292 | 1.00 | 112.60 | F |
| ATOM | 19673 | N | ASN | F | 204 | 43.212 | 13.232 | 6.237 | 1.00 | 80.80 | F |
| ATOM | 19674 | CA | ASN | F | 204 | 43.312 | 11.813 | 6.572 | 1.00 | 80.80 | F |
| ATOM | 19675 | CB | ASN | F | 204 | 42.265 | 10.982 | 5.806 | 1.00 | 113.91 | F |
| ATOM | 19676 | CG | ASN | F | 204 | 42.505 | 9.476 | 5.922 | 1.00 | 113.91 | F |
| ATOM | 19677 | OD1 | ASN | F | 204 | 43.517 | 8.977 | 5.441 | 1.00 | 113.91 | F |
| ATOM | 19678 | ND2 | ASN | F | 204 | 41.596 | 8.756 | 6.578 | 1.00 | 113.91 | F |
| ATOM | 19679 | C | ASN | F | 204 | 44.722 | 11.445 | 6.128 | 1.00 | 80.80 | F |
| ATOM | 19680 | O | ASN | F | 204 | 44.969 | 11.241 | 4.946 | 1.00 | 80.80 | F |
| ATOM | 19681 | N | ILE | F | 205 | 45.651 | 11.381 | 7.076 | 1.00 | 98.70 | F |
| ATOM | 19682 | CA | ILE | F | 205 | 47.041 | 11.071 | 6.757 | 1.00 | 98.70 | F |
| ATOM | 19683 | CB | ILE | F | 205 | 47.916 | 11.054 | 8.026 | 1.00 | 68.31 | F |
| ATOM | 19684 | CG2 | ILE | F | 205 | 47.792 | 9.716 | 8.729 | 1.00 | 68.31 | F |
| ATOM | 19685 | CG1 | ILE | F | 205 | 49.372 | 11.314 | 7.644 | 1.00 | 68.31 | F |
| ATOM | 19686 | CD1 | ILE | F | 205 | 50.230 | 11.758 | 8.807 | 1.00 | 68.31 | F |
| ATOM | 19687 | C | ILE | F | 205 | 47.244 | 9.758 | 6.001 | 1.00 | 98.70 | F |
| ATOM | 19688 | O | ILE | F | 205 | 48.298 | 9.541 | 5.404 | 1.00 | 98.70 | F |
| ATOM | 19689 | N | SER | F | 206 | 46.243 | 8.884 | 6.024 | 1.00 | 99.02 | F |
| ATOM | 19690 | CA | SER | F | 206 | 46.345 | 7.612 | 5.316 | 1.00 | 99.02 | F |
| ATOM | 19691 | CB | SER | F | 206 | 45.281 | 6.634 | 5.807 | 1.00 | 97.09 | F |
| ATOM | 19692 | OG | SER | F | 206 | 45.511 | 6.292 | 7.159 | 1.00 | 97.09 | F |
| ATOM | 19693 | C | SER | F | 206 | 46.184 | 7.825 | 3.818 | 1.00 | 99.02 | F |
| ATOM | 19694 | O | SER | F | 206 | 46.605 | 7.000 | 3.016 | 1.00 | 99.02 | F |
| ATOM | 19695 | N | ARG | F | 207 | 45.570 | 8.940 | 3.443 | 1.00 | 86.38 | F |
| ATOM | 19696 | CA | ARG | F | 207 | 45.369 | 9.250 | 2.038 | 1.00 | 86.38 | F |
| ATOM | 19697 | CB | ARG | F | 207 | 44.032 | 9.969 | 1.832 | 1.00 | 103.28 | F |
| ATOM | 19698 | CG | ARG | F | 207 | 42.828 | 9.052 | 1.953 | 1.00 | 103.28 | F |
| ATOM | 19699 | CD | ARG | F | 207 | 41.678 | 9.535 | 1.088 | 1.00 | 103.28 | F |
| ATOM | 19700 | NE | ARG | F | 207 | 40.981 | 10.685 | 1.656 | 1.00 | 103.28 | F |
| ATOM | 19701 | CZ | ARG | F | 207 | 40.312 | 11.576 | 0.932 | 1.00 | 103.28 | F |
| ATOM | 19702 | NH1 | ARG | F | 207 | 40.257 | 11.451 | −0.387 | 1.00 | 103.28 | F |
| ATOM | 19703 | NH2 | ARG | F | 207 | 39.690 | 12.586 | 1.525 | 1.00 | 103.28 | F |
| ATOM | 19704 | C | ARG | F | 207 | 46.502 | 10.100 | 1.486 | 1.00 | 86.38 | F |
| ATOM | 19705 | O | ARG | F | 207 | 46.406 | 10.611 | 0.374 | 1.00 | 86.38 | F |
| ATOM | 19706 | N | SER | F | 208 | 47.574 | 10.253 | 2.263 | 1.00 | 73.01 | F |
| ATOM | 19707 | CA | SER | F | 208 | 48.728 | 11.038 | 1.825 | 1.00 | 73.01 | F |
| ATOM | 19708 | CB | SER | F | 208 | 48.853 | 12.324 | 2.631 | 1.00 | 87.33 | F |
| ATOM | 19709 | OG | SER | F | 208 | 49.916 | 13.106 | 2.123 | 1.00 | 87.33 | F |
| ATOM | 19710 | C | SER | F | 208 | 50.008 | 10.232 | 1.965 | 1.00 | 73.01 | F |
| ATOM | 19711 | O | SER | F | 208 | 50.853 | 10.515 | 2.814 | 1.00 | 73.01 | F |
| ATOM | 19712 | N | PRO | F | 209 | 50.166 | 9.219 | 1.110 | 1.00 | 69.79 | F |
| ATOM | 19713 | CD | PRO | F | 209 | 49.232 | 8.966 | 0.002 | 1.00 | 88.90 | F |
| ATOM | 19714 | CA | PRO | F | 209 | 51.299 | 8.297 | 1.041 | 1.00 | 69.79 | F |
| ATOM | 19715 | CB | PRO | F | 209 | 51.088 | 7.598 | −0.297 | 1.00 | 88.90 | F |
| ATOM | 19716 | CG | PRO | F | 209 | 49.616 | 7.574 | −0.420 | 1.00 | 88.90 | F |
| ATOM | 19717 | C | PRO | F | 209 | 52.664 | 8.963 | 1.102 | 1.00 | 69.79 | F |
| ATOM | 19718 | O | PRO | F | 209 | 53.633 | 8.363 | 1.567 | 1.00 | 69.79 | F |
| ATOM | 19719 | N | HIS | F | 210 | 52.744 | 10.202 | 0.634 | 1.00 | 67.08 | F |
| ATOM | 19720 | CA | HIS | F | 210 | 54.021 | 10.885 | 0.605 | 1.00 | 67.08 | F |
| ATOM | 19721 | CB | HIS | F | 210 | 54.158 | 11.597 | −0.733 | 1.00 | 68.07 | F |
| ATOM | 19722 | CG | HIS | F | 210 | 54.167 | 10.655 | −1.894 | 1.00 | 68.07 | F |
| ATOM | 19723 | CD2 | HIS | F | 210 | 53.223 | 10.372 | −2.823 | 1.00 | 68.07 | F |
| ATOM | 19724 | ND1 | HIS | F | 210 | 55.222 | 9.804 | −2.149 | 1.00 | 68.07 | F |
| ATOM | 19725 | CE1 | HIS | F | 210 | 54.927 | 9.038 | −3.185 | 1.00 | 68.07 | F |
| ATOM | 19726 | NE2 | HIS | F | 210 | 53.720 | 9.362 | −3.611 | 1.00 | 68.07 | F |
| ATOM | 19727 | C | HIS | F | 210 | 54.322 | 11.810 | 1.769 | 1.00 | 67.08 | F |
| ATOM | 19728 | O | HIS | F | 210 | 55.314 | 12.537 | 1.753 | 1.00 | 67.08 | F |
| ATOM | 19729 | N | THR | F | 211 | 53.462 | 11.790 | 2.781 | 1.00 | 74.83 | F |
| ATOM | 19730 | CA | THR | F | 211 | 53.708 | 12.586 | 3.973 | 1.00 | 74.83 | F |
| ATOM | 19731 | CB | THR | F | 211 | 52.417 | 12.904 | 4.723 | 1.00 | 65.95 | F |
| ATOM | 19732 | OG1 | THR | F | 211 | 51.579 | 13.718 | 3.896 | 1.00 | 65.95 | F |
| ATOM | 19733 | CG2 | THR | F | 211 | 52.725 | 13.658 | 6.007 | 1.00 | 65.95 | F |
| ATOM | 19734 | C | THR | F | 211 | 54.549 | 11.619 | 4.796 | 1.00 | 74.83 | F |
| ATOM | 19735 | O | THR | F | 211 | 54.143 | 10.481 | 5.014 | 1.00 | 74.83 | F |
| ATOM | 19736 | N | PRO | F | 212 | 55.738 | 12.050 | 5.245 | 1.00 | 81.46 | F |
| ATOM | 19737 | CD | PRO | F | 212 | 56.262 | 13.423 | 5.110 | 1.00 | 66.17 | F |
| ATOM | 19738 | CA | PRO | F | 212 | 56.649 | 11.209 | 6.040 | 1.00 | 81.46 | F |
| ATOM | 19739 | CB | PRO | F | 212 | 57.681 | 12.210 | 6.549 | 1.00 | 66.17 | F |
| ATOM | 19740 | CG | PRO | F | 212 | 57.721 | 13.242 | 5.433 | 1.00 | 66.17 | F |
| ATOM | 19741 | C | PRO | F | 212 | 55.996 | 10.415 | 7.175 | 1.00 | 81.46 | F |
| ATOM | 19742 | O | PRO | F | 212 | 55.113 | 10.922 | 7.863 | 1.00 | 81.46 | F |
| ATOM | 19743 | N | GLY | F | 213 | 56.438 | 9.172 | 7.366 | 1.00 | 77.47 | F |
| ATOM | 19744 | CA | GLY | F | 213 | 55.898 | 8.331 | 8.429 | 1.00 | 77.47 | F |
| ATOM | 19745 | C | GLY | F | 213 | 56.571 | 8.576 | 9.775 | 1.00 | 77.47 | F |
| ATOM | 19746 | O | GLY | F | 213 | 57.467 | 9.417 | 9.881 | 1.00 | 77.47 | F |
| ATOM | 19747 | N | ASN | F | 214 | 56.156 | 7.839 | 10.803 | 1.00 | 96.63 | F |
| ATOM | 19748 | CA | ASN | F | 214 | 56.716 | 7.999 | 12.151 | 1.00 | 96.63 | F |
| ATOM | 19749 | CB | ASN | F | 214 | 58.214 | 7.702 | 12.194 | 1.00 | 105.27 | F |
| ATOM | 19750 | CG | ASN | F | 214 | 58.598 | 6.549 | 11.324 | 1.00 | 99.44 | F |
| ATOM | 19751 | OD1 | ASN | F | 214 | 57.749 | 5.763 | 10.917 | 1.00 | 99.44 | F |
| ATOM | 19752 | ND2 | ASN | F | 214 | 59.888 | 6.429 | 11.032 | 1.00 | 99.44 | F |
| ATOM | 19753 | C | ASN | F | 214 | 56.526 | 9.437 | 12.574 | 1.00 | 96.63 | F |
| ATOM | 19754 | O | ASN | F | 214 | 57.473 | 10.091 | 13.025 | 1.00 | 96.63 | F |
| ATOM | 19755 | N | ASP | F | 215 | 55.302 | 9.930 | 12.423 | 1.00 | 80.65 | F |
| ATOM | 19756 | CA | ASP | F | 215 | 55.000 | 11.303 | 12.777 | 1.00 | 80.65 | F |
| ATOM | 19757 | CB | ASP | F | 215 | 54.912 | 11.453 | 14.292 | 1.00 | 171.65 | F |
| ATOM | 19758 | CG | ASP | F | 215 | 53.899 | 10.518 | 14.899 | 1.00 | 93.03 | F |
| ATOM | 19759 | OD1 | ASP | F | 215 | 52.723 | 10.557 | 14.480 | 1.00 | 93.03 | F |
| ATOM | 19760 | OD2 | ASP | F | 215 | 54.282 | 9.741 | 15.793 | 1.00 | 93.03 | F |
| ATOM | 19761 | C | ASP | F | 215 | 56.085 | 12.210 | 12.223 | 1.00 | 80.65 | F |
| ATOM | 19762 | O | ASP | F | 215 | 56.825 | 12.852 | 12.974 | 1.00 | 80.65 | F |
| ATOM | 19763 | N | TYR | F | 216 | 56.190 | 12.224 | 10.898 | 1.00 | 53.58 | F |
| ATOM | 19764 | CA | TYR | F | 216 | 57.156 | 13.062 | 10.198 | 1.00 | 53.58 | F |
| ATOM | 19765 | CB | TYR | F | 216 | 56.770 | 14.540 | 10.349 | 1.00 | 107.15 | F |
| ATOM | 19766 | CG | TYR | F | 216 | 55.337 | 14.844 | 9.972 | 1.00 | 85.90 | F |
| ATOM | 19767 | CD1 | TYR | F | 216 | 54.278 | 14.373 | 10.744 | 1.00 | 85.90 | F |
| ATOM | 19768 | CE1 | TYR | F | 216 | 52.957 | 14.625 | 10.391 | 1.00 | 85.90 | F |
| ATOM | 19769 | CD2 | TYR | F | 216 | 55.039 | 15.584 | 8.829 | 1.00 | 85.90 | F |
| ATOM | 19770 | CE2 | TYR | F | 216 | 53.721 | 15.845 | 8.466 | 1.00 | 85.90 | F |
| ATOM | 19771 | CZ | TYR | F | 216 | 52.683 | 15.360 | 9.252 | 1.00 | 85.90 | F |
| ATOM | 19772 | OH | TYR | F | 216 | 51.377 | 15.607 | 8.894 | 1.00 | 85.90 | F |
| ATOM | 19773 | C | TYR | F | 216 | 58.598 | 12.878 | 10.650 | 1.00 | 53.58 | F |
| ATOM | 19774 | O | TYR | F | 216 | 59.370 | 13.845 | 10.658 | 1.00 | 53.58 | F |
| ATOM | 19775 | N | ASN | F | 217 | 58.962 | 11.647 | 11.013 | 1.00 | 74.71 | F |
| ATOM | 19776 | CA | ASN | F | 217 | 60.320 | 11.351 | 11.471 | 1.00 | 74.71 | F |
| ATOM | 19777 | CB | ASN | F | 217 | 61.311 | 11.469 | 10.311 | 1.00 | 88.62 | F |
| ATOM | 19778 | CG | ASN | F | 217 | 61.020 | 10.492 | 9.195 | 1.00 | 83.35 | F |
| ATOM | 19779 | OD1 | ASN | F | 217 | 60.834 | 10.885 | 8.040 | 1.00 | 83.35 | F |
| ATOM | 19780 | ND2 | ASN | F | 217 | 60.980 | 9.206 | 9.531 | 1.00 | 83.35 | F |
| ATOM | 19781 | C | ASN | F | 217 | 60.706 | 12.340 | 12.563 | 1.00 | 74.71 | F |
| ATOM | 19782 | O | ASN | F | 217 | 61.858 | 12.774 | 12.646 | 1.00 | 74.71 | F |
| ATOM | 19783 | N | PHE | F | 218 | 59.739 | 12.692 | 13.406 | 1.00 | 78.58 | F |
| ATOM | 19784 | CA | PHE | F | 218 | 59.989 | 13.658 | 14.467 | 1.00 | 78.58 | F |
| ATOM | 19785 | CB | PHE | F | 218 | 58.722 | 13.947 | 15.262 | 1.00 | 71.53 | F |
| ATOM | 19766 | CG | PHE | F | 218 | 58.910 | 15.016 | 16.285 | 1.00 | 68.05 | F |
| ATOM | 19787 | CD1 | PHE | F | 218 | 58.838 | 16.358 | 15.931 | 1.00 | 68.05 | F |
| ATOM | 19788 | CD2 | PHE | F | 218 | 59.228 | 14.685 | 17.595 | 1.00 | 68.05 | F |
| ATOM | 19789 | CE1 | PHE | F | 218 | 59.084 | 17.363 | 16.873 | 1.00 | 68.05 | F |
| ATOM | 19790 | CE2 | PHE | F | 218 | 59.478 | 15.680 | 18.543 | 1.00 | 68.05 | F |
| ATOM | 19791 | CZ | PHE | F | 218 | 59.405 | 17.022 | 18.177 | 1.00 | 68.05 | F |
| ATOM | 19792 | C | PHE | F | 218 | 61.082 | 13.261 | 15.442 | 1.00 | 78.58 | F |
| ATOM | 19793 | O | PHE | F | 218 | 62.002 | 14.033 | 15.690 | 1.00 | 78.58 | F |
| ATOM | 19794 | N | ASN | F | 219 | 60.974 | 12.068 | 16.014 | 1.00 | 71.57 | F |
| ATOM | 19795 | CA | ASN | F | 219 | 61.983 | 11.628 | 16.963 | 1.00 | 71.57 | F |
| ATOM | 19796 | CB | ASN | F | 219 | 61.716 | 10.195 | 17.415 | 1.00 | 71.18 | F |
| ATOM | 19797 | CG | ASN | F | 219 | 60.358 | 10.037 | 18.059 | 1.00 | 62.81 | F |
| ATOM | 19798 | OD1 | ASN | F | 219 | 59.875 | 10.940 | 18.744 | 1.00 | 62.81 | F |
| ATOM | 19799 | ND2 | ASN | F | 219 | 59.738 | 8.884 | 17.856 | 1.00 | 62.81 | F |
| ATOM | 19800 | C | ASN | F | 219 | 63.362 | 11.727 | 16.334 | 1.00 | 71.57 | F |
| ATOM | 19801 | O | ASN | F | 219 | 64.301 | 12.210 | 16.968 | 1.00 | 71.57 | F |
| ATOM | 19802 | N | PHE | F | 220 | 63.481 | 11.281 | 15.084 | 1.00 | 58.80 | F |
| ATOM | 19803 | CA | PHE | F | 220 | 64.762 | 11.341 | 14.387 | 1.00 | 58.80 | F |
| ATOM | 19804 | CB | PHE | F | 220 | 64.624 | 10.827 | 12.951 | 1.00 | 66.19 | F |
| ATOM | 19805 | CG | PHE | F | 220 | 65.816 | 11.146 | 12.084 | 1.00 | 59.17 | F |
| ATOM | 19806 | CD1 | PHE | F | 220 | 66.953 | 10.338 | 12.110 | 1.00 | 59.17 | F |
| ATOM | 19807 | CD2 | PHE | F | 220 | 65.823 | 12.293 | 11.284 | 1.00 | 59.17 | F |
| ATOM | 19808 | CE1 | PHE | F | 220 | 68.081 | 10.665 | 11.356 | 1.00 | 59.17 | F |
| ATOM | 19809 | CE2 | PHE | F | 220 | 66.946 | 12.630 | 10.528 | 1.00 | 59.17 | F |
| ATOM | 19810 | CZ | PHE | F | 220 | 68.079 | 11.813 | 10.564 | 1.00 | 59.17 | F |
| ATOM | 19811 | C | PHE | F | 220 | 65.308 | 12.769 | 14.345 | 1.00 | 58.80 | F |
| ATOM | 19812 | O | PHE | F | 220 | 66.433 | 13.027 | 14.774 | 1.00 | 58.80 | F |
| ATOM | 19813 | N | TRP | F | 221 | 64.507 | 13.689 | 13.813 | 1.00 | 70.44 | F |
| ATOM | 19814 | CA | TRP | F | 221 | 64.924 | 15.082 | 13.690 | 1.00 | 70.44 | F |
| ATOM | 19815 | CB | TRP | F | 221 | 63.898 | 15.865 | 12.860 | 1.00 | 63.79 | F |
| ATOM | 19816 | CG | TRP | F | 221 | 63.954 | 15.528 | 11.399 | 1.00 | 56.94 | F |
| ATOM | 19817 | CD2 | TRP | F | 221 | 65.070 | 15.726 | 10.526 | 1.00 | 56.94 | F |
| ATOM | 19818 | CE2 | TRP | F | 221 | 64.705 | 15.219 | 9.262 | 1.00 | 56.94 | F |
| ATOM | 19819 | CE3 | TRP | F | 221 | 66.349 | 16.282 | 10.692 | 1.00 | 56.94 | F |
| ATOM | 19820 | CD1 | TRP | F | 221 | 62.979 | 14.929 | 10.650 | 1.00 | 56.94 | F |
| ATOM | 19821 | NE1 | TRP | F | 221 | 63.424 | 14.739 | 9.364 | 1.00 | 56.94 | F |
| ATOM | 19822 | CZ2 | TRP | F | 221 | 65.570 | 15.247 | 8.170 | 1.00 | 56.94 | F |
| ATOM | 19823 | CZ3 | TRP | F | 221 | 67.208 | 16.310 | 9.609 | 1.00 | 56.94 | F |
| ATOM | 19824 | CH2 | TRP | F | 221 | 66.814 | 15.794 | 8.361 | 1.00 | 56.94 | F |
| ATOM | 19825 | C | TRP | F | 221 | 65.163 | 15.781 | 15.022 | 1.00 | 70.44 | F |
| ATOM | 19826 | O | TRP | F | 221 | 66.184 | 16.446 | 15.209 | 1.00 | 70.44 | F |
| ATOM | 19827 | N | TYR | F | 222 | 64.224 | 15.636 | 15.950 | 1.00 | 65.01 | F |
| ATOM | 19828 | CA | TYR | F | 222 | 64.371 | 16.266 | 17.254 | 1.00 | 65.01 | F |
| ATOM | 19829 | CB | TYR | F | 222 | 63.313 | 15.756 | 18.227 | 1.00 | 105.48 | F |
| ATOM | 19830 | CG | TYR | F | 222 | 63.600 | 16.204 | 19.631 | 1.00 | 104.05 | F |
| ATOM | 19831 | CD1 | TYR | F | 222 | 63.331 | 17.507 | 20.030 | 1.00 | 104.05 | F |
| ATOM | 19832 | CE1 | TYR | F | 222 | 63.673 | 17.951 | 21.295 | 1.00 | 104.05 | F |
| ATOM | 19833 | CD2 | TYR | F | 222 | 64.219 | 15.350 | 20.538 | 1.00 | 104.05 | F |
| ATOM | 19834 | CE2 | TYR | F | 222 | 64.570 | 15.781 | 21.805 | 1.00 | 104.05 | F |
| ATOM | 19835 | CZ | TYR | F | 222 | 64.296 | 17.081 | 22.179 | 1.00 | 104.05 | F |
| ATOM | 19836 | OH | TYR | F | 222 | 64.657 | 17.511 | 23.435 | 1.00 | 104.05 | F |
| ATOM | 19837 | C | TYR | F | 222 | 65.742 | 15.944 | 17.824 | 1.00 | 65.01 | F |
| ATOM | 19838 | O | TYR | F | 222 | 66.506 | 16.832 | 18.202 | 1.00 | 65.01 | F |
| ATOM | 19839 | N | SER | F | 223 | 66.036 | 14.653 | 17.876 | 1.00 | 88.80 | F |
| ATOM | 19840 | CA | SER | F | 223 | 67.294 | 14.182 | 18.406 | 1.00 | 88.80 | F |
| ATOM | 19841 | CB | SER | F | 223 | 67.302 | 12.665 | 18.425 | 1.00 | 62.43 | F |
| ATOM | 19842 | OG | SER | F | 223 | 68.582 | 12.200 | 18.795 | 1.00 | 61.05 | F |
| ATOM | 19843 | C | SER | F | 223 | 68.511 | 14.685 | 17.645 | 1.00 | 88.80 | F |
| ATOM | 19844 | O | SER | F | 223 | 69.459 | 15.179 | 18.253 | 1.00 | 88.80 | F |
| ATOM | 19845 | N | LEU | F | 224 | 68.493 | 14.566 | 16.321 | 1.00 | 62.60 | F |
| ATOM | 19846 | CA | LEU | F | 224 | 69.630 | 15.000 | 15.525 | 1.00 | 62.60 | F |
| ATOM | 19847 | CB | LEU | F | 224 | 69.491 | 14.521 | 14.080 | 1.00 | 83.83 | F |
| ATOM | 19848 | CG | LEU | F | 224 | 70.647 | 14.932 | 13.162 | 1.00 | 61.07 | F |
| ATOM | 19849 | CD1 | LEU | F | 224 | 71.985 | 14.487 | 13.740 | 1.00 | 61.07 | F |
| ATOM | 19850 | CD2 | LEU | F | 224 | 70.419 | 14.326 | 11.794 | 1.00 | 61.07 | F |
| ATOM | 19851 | C | LEU | F | 224 | 69.804 | 16.507 | 15.557 | 1.00 | 62.60 | F |
| ATOM | 19852 | O | LEU | F | 224 | 70.928 | 17.009 | 15.464 | 1.00 | 62.60 | F |
| ATOM | 19853 | N | MET | F | 225 | 68.695 | 17.230 | 15.684 | 1.00 | 74.95 | F |
| ATOM | 19854 | CA | MET | F | 225 | 68.761 | 18.682 | 15.745 | 1.00 | 74.95 | F |
| ATOM | 19855 | CB | MET | F | 225 | 67.399 | 19.283 | 15.462 | 1.00 | 110.16 | F |
| ATOM | 19856 | CG | MET | F | 225 | 66.986 | 19.082 | 14.038 | 1.00 | 78.88 | F |
| ATOM | 19857 | SD | MET | F | 225 | 65.487 | 19.943 | 13.649 | 1.00 | 78.88 | F |
| ATOM | 19858 | CE | MET | F | 225 | 66.032 | 21.663 | 13.906 | 1.00 | 78.88 | F |
| ATOM | 19859 | C | MET | F | 225 | 69.245 | 19.099 | 17.120 | 1.00 | 74.95 | F |
| ATOM | 19860 | O | MET | F | 225 | 69.972 | 20.080 | 17.265 | 1.00 | 74.95 | F |
| ATOM | 19861 | N | LYS | F | 226 | 68.837 | 18.349 | 18.134 | 1.00 | 84.52 | F |
| ATOM | 19862 | CA | LYS | F | 226 | 69.272 | 18.639 | 19.486 | 1.00 | 84.52 | F |
| ATOM | 19863 | CB | LYS | F | 226 | 68.661 | 17.635 | 20.460 | 1.00 | 140.11 | F |
| ATOM | 19864 | CG | LYS | F | 226 | 69.061 | 17.860 | 21.899 | 1.00 | 91.57 | F |
| ATOM | 19865 | CD | LYS | F | 226 | 68.553 | 16.736 | 22.770 | 1.00 | 91.57 | F |
| ATOM | 19866 | CE | LYS | F | 226 | 69.159 | 16.812 | 24.158 | 1.00 | 91.57 | F |
| ATOM | 19867 | NZ | LYS | F | 226 | 68.701 | 15.676 | 25.002 | 1.00 | 91.57 | F |
| ATOM | 19868 | C | LYS | F | 226 | 70.798 | 18.527 | 19.492 | 1.00 | 84.52 | F |
| ATOM | 19869 | O | LYS | F | 226 | 71.492 | 19.397 | 20.015 | 1.00 | 84.52 | F |
| ATOM | 19870 | N | GLU | F | 227 | 71.310 | 17.456 | 18.888 | 1.00 | 78.09 | F |
| ATOM | 19871 | CA | GLU | F | 227 | 72.749 | 17.226 | 18.807 | 1.00 | 78.09 | F |
| ATOM | 19872 | CB | GLU | F | 227 | 73.045 | 15.938 | 18.034 | 1.00 | 140.66 | F |
| ATOM | 19873 | CG | GLU | F | 227 | 74.521 | 15.569 | 18.010 | 1.00 | 116.46 | F |
| ATOM | 19874 | CD | GLU | F | 227 | 74.850 | 14.506 | 16.978 | 1.00 | 116.46 | F |
| ATOM | 19875 | OE1 | GLU | F | 227 | 74.186 | 13.444 | 16.967 | 1.00 | 116.46 | F |
| ATOM | 19876 | OE2 | GLU | F | 227 | 75.782 | 14.734 | 16.180 | 1.00 | 116.46 | F |
| ATOM | 19877 | C | GLU | F | 227 | 73.427 | 18.390 | 18.098 | 1.00 | 78.09 | F |
| ATOM | 19878 | O | GLU | F | 227 | 74.432 | 18.913 | 18.568 | 1.00 | 78.09 | F |
| ATOM | 19879 | N | THR | F | 228 | 72.868 | 18.786 | 16.958 | 1.00 | 94.23 | F |
| ATOM | 19880 | CA | THR | F | 228 | 73.413 | 19.881 | 16.168 | 1.00 | 94.23 | F |
| ATOM | 19881 | CB | THR | F | 228 | 72.609 | 20.085 | 14.877 | 1.00 | 100.40 | F |
| ATOM | 19882 | OG1 | THR | F | 228 | 72.622 | 18.875 | 14.113 | 1.00 | 86.39 | F |
| ATOM | 19883 | CG2 | THR | F | 228 | 73.212 | 21.211 | 14.048 | 1.00 | 86.39 | F |
| ATOM | 19884 | C | THR | F | 228 | 73.425 | 21.199 | 16.930 | 1.00 | 94.23 | F |
| ATOM | 19885 | O | THR | F | 228 | 74.372 | 21.978 | 16.818 | 1.00 | 94.23 | F |
| ATOM | 19886 | N | LEU | F | 229 | 72.369 | 21.455 | 17.695 | 1.00 | 84.00 | F |
| ATOM | 19887 | CA | LEU | F | 229 | 72.293 | 22.690 | 18.454 | 1.00 | 84.00 | F |
| ATOM | 19888 | CB | LEU | F | 229 | 70.912 | 22.838 | 19.089 | 1.00 | 95.47 | F |
| ATOM | 19889 | CG | LEU | F | 229 | 70.669 | 24.126 | 19.878 | 1.00 | 67.35 | F |
| ATOM | 19890 | CD1 | LEU | F | 229 | 71.224 | 25.337 | 19.131 | 1.00 | 67.35 | F |
| ATOM | 19891 | CD2 | LEU | F | 229 | 69.171 | 24.270 | 20.119 | 1.00 | 67.35 | F |
| ATOM | 19892 | C | LEU | F | 229 | 73.379 | 22.724 | 19.521 | 1.00 | 84.00 | F |
| ATOM | 19893 | O | LEU | F | 229 | 74.016 | 23.755 | 19.725 | 1.00 | 84.00 | F |
| ATOM | 19894 | N | GLU | F | 230 | 73.600 | 21.594 | 20.188 | 1.00 | 91.44 | F |
| ATOM | 19895 | CA | GLU | F | 230 | 74.621 | 21.509 | 21.225 | 1.00 | 91.44 | F |
| ATOM | 19896 | CB | GLU | F | 230 | 74.607 | 20.124 | 21.873 | 1.00 | 146.68 | F |
| ATOM | 19897 | CG | GLU | F | 230 | 73.380 | 19.879 | 22.728 | 1.00 | 111.14 | F |
| ATOM | 19898 | CD | GLU | F | 230 | 73.395 | 18.525 | 23.407 | 1.00 | 111.14 | F |
| ATOM | 19899 | OE1 | GLU | F | 230 | 74.415 | 18.201 | 24.052 | 1.00 | 111.14 | F |
| ATOM | 19900 | OE2 | GLU | F | 230 | 72.386 | 17.790 | 23.306 | 1.00 | 111.14 | F |
| ATOM | 19901 | C | GLU | F | 230 | 76.003 | 21.809 | 20.655 | 1.00 | 91.44 | F |
| ATOM | 19902 | O | GLU | F | 230 | 76.757 | 22.591 | 21.229 | 1.00 | 91.44 | F |
| ATOM | 19903 | N | GLU | F | 231 | 76.330 | 21.195 | 19.523 | 1.00 | 86.27 | F |
| ATOM | 19904 | CA | GLU | F | 231 | 77.624 | 21.415 | 18.880 | 1.00 | 86.27 | F |
| ATOM | 19905 | CB | GLU | F | 231 | 77.742 | 20.552 | 17.620 | 1.00 | 151.53 | F |
| ATOM | 19906 | CG | GLU | F | 231 | 77.529 | 19.068 | 17.855 | 1.00 | 127.77 | F |
| ATOM | 19907 | CD | GLU | F | 231 | 78.626 | 18.443 | 18.692 | 1.00 | 127.77 | F |
| ATOM | 19908 | OE1 | GLU | F | 231 | 78.891 | 18.950 | 19.801 | 1.00 | 127.77 | F |
| ATOM | 19909 | OE2 | GLU | F | 231 | 79.221 | 17.441 | 18.242 | 1.00 | 127.77 | F |
| ATOM | 19910 | C | GLU | F | 231 | 77.770 | 22.890 | 18.498 | 1.00 | 86.27 | F |
| ATOM | 19911 | O | GLU | F | 231 | 78.812 | 23.509 | 18.726 | 1.00 | 86.27 | F |
| ATOM | 19912 | N | ILE | F | 232 | 76.716 | 23.446 | 17.912 | 1.00 | 122.62 | F |
| ATOM | 19913 | CA | ILE | F | 232 | 76.721 | 24.842 | 17.495 | 1.00 | 122.62 | F |
| ATOM | 19914 | CB | ILE | F | 232 | 75.370 | 25.229 | 16.843 | 1.00 | 124.23 | F |
| ATOM | 19915 | CG2 | ILE | F | 232 | 75.246 | 26.735 | 16.743 | 1.00 | 94.28 | F |
| ATOM | 19916 | CG1 | ILE | F | 232 | 75.260 | 24.602 | 15.452 | 1.00 | 94.28 | F |
| ATOM | 19917 | CD1 | ILE | F | 232 | 73.957 | 24.925 | 14.741 | 1.00 | 94.28 | F |
| ATOM | 19918 | C | ILE | F | 232 | 76.999 | 25.789 | 18.660 | 1.00 | 122.62 | F |
| ATOM | 19919 | O | ILE | F | 232 | 77.627 | 26.833 | 18.485 | 1.00 | 122.62 | F |
| ATOM | 19920 | N | ASN | F | 233 | 76.539 | 25.423 | 19.851 | 1.00 | 103.77 | F |
| ATOM | 19921 | CA | ASN | F | 233 | 76.740 | 26.274 | 21.015 | 1.00 | 103.77 | F |
| ATOM | 19922 | CB | ASN | F | 233 | 75.649 | 26.017 | 22.054 | 1.00 | 116.86 | F |
| ATOM | 19923 | CG | ASN | F | 233 | 74.294 | 26.530 | 21.611 | 1.00 | 105.78 | F |
| ATOM | 19924 | OD1 | ASN | F | 233 | 74.202 | 27.531 | 20.900 | 1.00 | 105.78 | F |
| ATOM | 19925 | ND2 | ASN | F | 233 | 73.233 | 25.858 | 22.046 | 1.00 | 105.78 | F |
| ATOM | 19926 | C | ASN | F | 233 | 78.109 | 26.178 | 21.682 | 1.00 | 103.77 | F |
| ATOM | 19927 | O | ASN | F | 233 | 78.433 | 27.000 | 22.540 | 1.00 | 103.77 | F |
| ATOM | 19928 | N | LYS | F | 234 | 78.914 | 25.190 | 21.302 | 1.00 | 105.62 | F |
| ATOM | 19929 | CA | LYS | F | 234 | 80.239 | 25.057 | 21.894 | 1.00 | 105.62 | F |
| ATOM | 19930 | CB | LYS | F | 234 | 80.875 | 23.731 | 21.490 | 1.00 | 82.95 | F |
| ATOM | 19931 | CG | LYS | F | 234 | 80.248 | 22.560 | 22.226 | 1.00 | 82.95 | F |
| ATOM | 19932 | CD | LYS | F | 234 | 80.900 | 21.229 | 21.890 | 1.00 | 82.95 | F |
| ATOM | 19933 | CE | LYS | F | 234 | 80.257 | 20.107 | 22.703 | 1.00 | 82.95 | F |
| ATOM | 19934 | NZ | LYS | F | 234 | 80.751 | 18.755 | 22.321 | 1.00 | 82.95 | F |
| ATOM | 19935 | C | LYS | F | 234 | 81.127 | 26.239 | 21.518 | 1.00 | 105.62 | F |
| ATOM | 19936 | O | LYS | F | 234 | 81.804 | 26.796 | 22.379 | 1.00 | 105.62 | F |
| ATOM | 19937 | N | ASN | F | 235 | 81.141 | 26.624 | 20.246 | 1.00 | 123.37 | F |
| ATOM | 19938 | CA | ASN | F | 235 | 81.917 | 27.797 | 19.849 | 1.00 | 123.37 | F |
| ATOM | 19939 | CB | ASN | F | 235 | 82.101 | 27.870 | 18.326 | 1.00 | 136.12 | F |
| ATOM | 19940 | CG | ASN | F | 235 | 82.785 | 29.161 | 17.880 | 1.00 | 136.12 | F |
| ATOM | 19941 | OD1 | ASN | F | 235 | 82.451 | 30.238 | 18.376 | 1.00 | 136.12 | F |
| ATOM | 19942 | ND2 | ASN | F | 235 | 83.728 | 29.068 | 16.942 | 1.00 | 136.12 | F |
| ATOM | 19943 | C | ASN | F | 235 | 81.013 | 28.931 | 20.327 | 1.00 | 123.37 | F |
| ATOM | 19944 | O | ASN | F | 235 | 79.938 | 29.152 | 19.773 | 1.00 | 123.37 | F |
| ATOM | 19945 | N | ASP | F | 236 | 81.442 | 29.635 | 21.366 | 1.00 | 147.23 | F |
| ATOM | 19946 | CA | ASP | F | 236 | 80.645 | 30.710 | 21.946 | 1.00 | 147.23 | F |
| ATOM | 19947 | CB | ASP | F | 236 | 81.356 | 31.251 | 23.191 | 1.00 | 180.48 | F |
| ATOM | 19948 | CG | ASP | F | 236 | 80.398 | 31.888 | 24.178 | 1.00 | 180.48 | F |
| ATOM | 19949 | OD1 | ASP | F | 236 | 79.866 | 32.976 | 23.879 | 1.00 | 180.48 | F |
| ATOM | 19950 | OD2 | ASP | F | 236 | 80.173 | 31.293 | 25.254 | 1.00 | 180.48 | F |
| ATOM | 19951 | C | ASP | F | 236 | 80.276 | 31.865 | 21.009 | 1.00 | 147.23 | F |
| ATOM | 19952 | O | ASP | F | 236 | 79.491 | 32.735 | 21.382 | 1.00 | 147.23 | F |
| ATOM | 19953 | N | SER | F | 237 | 80.823 | 31.879 | 19.797 | 1.00 | 127.88 | F |
| ATOM | 19954 | CA | SER | F | 237 | 80.511 | 32.952 | 18.851 | 1.00 | 127.88 | F |
| ATOM | 19955 | CB | SER | F | 237 | 81.739 | 33.303 | 18.002 | 1.00 | 86.24 | F |
| ATOM | 19956 | OG | SER | F | 237 | 81.973 | 32.327 | 17.003 | 1.00 | 86.24 | F |
| ATOM | 19957 | C | SER | F | 237 | 79.351 | 32.578 | 17.928 | 1.00 | 127.88 | F |
| ATOM | 19958 | O | SER | F | 237 | 78.999 | 33.335 | 17.021 | 1.00 | 127.88 | F |
| ATOM | 19959 | N | THR | F | 238 | 78.763 | 31.408 | 18.161 | 1.00 | 120.54 | F |
| ATOM | 19960 | CA | THR | F | 238 | 77.643 | 30.929 | 17.355 | 1.00 | 120.54 | F |
| ATOM | 19961 | CB | THR | F | 238 | 77.981 | 29.607 | 16.642 | 1.00 | 99.52 | F |
| ATOM | 19962 | OG1 | THR | F | 238 | 79.279 | 29.702 | 16.047 | 1.00 | 99.52 | F |
| ATOM | 19963 | CG2 | THR | F | 238 | 76.962 | 29.321 | 15.548 | 1.00 | 99.52 | F |
| ATOM | 19964 | C | THR | F | 238 | 76.436 | 30.679 | 18.245 | 1.00 | 120.54 | F |
| ATOM | 19965 | O | THR | F | 238 | 75.298 | 30.718 | 17.781 | 1.00 | 120.54 | F |
| ATOM | 19966 | N | LYS | F | 239 | 76.702 | 30.417 | 19.522 | 1.00 | 107.95 | F |
| ATOM | 19967 | CA | LYS | F | 239 | 75.664 | 30.149 | 20.513 | 1.00 | 107.95 | F |
| ATOM | 19968 | CB | LYS | F | 239 | 76.082 | 30.724 | 21.872 | 1.00 | 147.96 | F |
| ATOM | 19969 | CG | LYS | F | 239 | 75.106 | 30.431 | 23.005 | 1.00 | 147.96 | F |
| ATOM | 19970 | CD | LYS | F | 239 | 75.700 | 30.796 | 24.358 | 1.00 | 147.96 | F |
| ATOM | 19971 | CE | LYS | F | 239 | 76.928 | 29.953 | 24.666 | 1.00 | 147.96 | F |
| ATOM | 19972 | NZ | LYS | F | 239 | 77.521 | 30.300 | 25.987 | 1.00 | 147.96 | F |
| ATOM | 19973 | C | LYS | F | 239 | 74.311 | 30.714 | 20.095 | 1.00 | 107.95 | F |
| ATOM | 19974 | O | LYS | F | 239 | 74.174 | 31.911 | 19.831 | 1.00 | 107.95 | F |
| ATOM | 19975 | N | THR | F | 240 | 73.313 | 29.840 | 20.031 | 1.00 | 73.05 | F |
| ATOM | 19976 | CA | THR | F | 240 | 71.974 | 30.242 | 19.626 | 1.00 | 73.05 | F |
| ATOM | 19977 | CB | THR | F | 240 | 71.666 | 29.791 | 18.189 | 1.00 | 105.96 | F |
| ATOM | 19978 | OG1 | THR | F | 240 | 72.690 | 30.264 | 17.305 | 1.00 | 105.96 | F |
| ATOM | 19979 | CG2 | THR | F | 240 | 70.322 | 30.341 | 17.742 | 1.00 | 105.96 | F |
| ATOM | 19980 | C | THR | F | 240 | 70.897 | 29.661 | 20.525 | 1.00 | 73.05 | F |
| ATOM | 19981 | O | THR | F | 240 | 71.085 | 28.616 | 21.156 | 1.00 | 73.05 | F |
| ATOM | 19982 | N | THR | F | 241 | 69.760 | 30.350 | 20.552 | 1.00 | 85.16 | F |
| ATOM | 19983 | CA | THR | F | 241 | 68.609 | 29.955 | 21.350 | 1.00 | 85.16 | F |
| ATOM | 19984 | CB | THR | F | 241 | 67.545 | 31.059 | 21.359 | 1.00 | 124.68 | F |
| ATOM | 19985 | OG1 | THR | F | 241 | 68.165 | 32.317 | 21.650 | 1.00 | 84.38 | F |
| ATOM | 19986 | CG2 | THR | F | 241 | 66.467 | 30.752 | 22.393 | 1.00 | 84.38 | F |
| ATOM | 19987 | C | THR | F | 241 | 67.951 | 28.715 | 20.775 | 1.00 | 85.16 | F |
| ATOM | 19988 | O | THR | F | 241 | 67.587 | 27.790 | 21.505 | 1.00 | 85.16 | F |
| ATOM | 19989 | N | SER | F | 242 | 67.799 | 28.710 | 19.454 | 1.00 | 110.51 | F |
| ATOM | 19990 | CA | SER | F | 242 | 67.143 | 27.605 | 18.774 | 1.00 | 110.51 | F |
| ATOM | 19991 | CB | SER | F | 242 | 65.643 | 27.886 | 18.683 | 1.00 | 132.23 | F |
| ATOM | 19992 | OG | SER | F | 242 | 65.409 | 29.057 | 17.916 | 1.00 | 104.75 | F |
| ATOM | 19993 | C | SER | F | 242 | 67.669 | 27.309 | 17.374 | 1.00 | 110.51 | F |
| ATOM | 19994 | O | SER | F | 242 | 68.459 | 28.064 | 16.805 | 1.00 | 110.51 | F |
| ATOM | 19995 | N | LEU | F | 243 | 67.186 | 26.196 | 16.830 | 1.00 | 73.66 | F |
| ATOM | 19996 | CA | LEU | F | 243 | 67.551 | 25.730 | 15.506 | 1.00 | 73.66 | F |
| ATOM | 19997 | CB | LEU | F | 243 | 68.536 | 24.574 | 15.633 | 1.00 | 80.49 | F |
| ATOM | 19998 | CG | LEU | F | 243 | 69.240 | 24.151 | 14.353 | 1.00 | 56.62 | F |
| ATOM | 19999 | CD1 | LEU | F | 243 | 70.015 | 25.333 | 13.781 | 1.00 | 56.62 | F |
| ATOM | 20000 | CD2 | LEU | F | 243 | 70.176 | 22.983 | 14.658 | 1.00 | 56.62 | F |
| ATOM | 20001 | C | LEU | F | 243 | 66.281 | 25.263 | 14.799 | 1.00 | 73.66 | F |
| ATOM | 20002 | O | LEU | F | 243 | 65.410 | 24.628 | 15.412 | 1.00 | 73.66 | F |
| ATOM | 20003 | N | SER | F | 244 | 66.170 | 25.587 | 13.514 | 1.00 | 72.83 | F |
| ATOM | 20004 | CA | SER | F | 244 | 64.995 | 25.192 | 12.740 | 1.00 | 72.83 | F |
| ATOM | 20005 | CB | SER | F | 244 | 64.092 | 26.401 | 12.486 | 1.00 | 116.10 | F |
| ATOM | 20006 | OG | SER | F | 244 | 63.522 | 26.878 | 13.686 | 1.00 | 118.25 | F |
| ATOM | 20007 | C | SER | F | 244 | 65.309 | 24.542 | 11.397 | 1.00 | 72.83 | F |
| ATOM | 20008 | O | SER | F | 244 | 66.241 | 24.940 | 10.696 | 1.00 | 72.83 | F |
| ATOM | 20009 | N | LEU | F | 245 | 64.516 | 23.534 | 11.050 | 1.00 | 76.20 | F |
| ATOM | 20010 | CA | LEU | F | 245 | 64.658 | 22.849 | 9.773 | 1.00 | 76.20 | F |
| ATOM | 20011 | CB | LEU | F | 245 | 64.701 | 21.336 | 9.965 | 1.00 | 87.08 | F |
| ATOM | 20012 | CG | LEU | F | 245 | 64.761 | 20.582 | 8.639 | 1.00 | 71.07 | F |
| ATOM | 20013 | CD1 | LEU | F | 245 | 65.976 | 21.044 | 7.848 | 1.00 | 71.07 | F |
| ATOM | 20014 | CD2 | LEU | F | 245 | 64.816 | 19.086 | 8.893 | 1.00 | 71.07 | F |
| ATOM | 20015 | C | LEU | F | 245 | 63.437 | 23.224 | 8.944 | 1.00 | 76.20 | F |
| ATOM | 20016 | O | LEU | F | 245 | 62.330 | 22.750 | 9.204 | 1.00 | 76.20 | F |
| ATOM | 20017 | N | ILE | F | 246 | 63.636 | 24.090 | 7.960 | 1.00 | 77.06 | F |
| ATOM | 20018 | CA | ILE | F | 246 | 62.534 | 24.537 | 7.117 | 1.00 | 77.06 | F |
| ATOM | 20019 | CB | ILE | F | 246 | 62.603 | 26.070 | 6.872 | 1.00 | 80.16 | F |
| ATOM | 20020 | CG2 | ILE | F | 246 | 61.397 | 26.540 | 6.077 | 1.00 | 63.20 | F |
| ATOM | 20021 | CG1 | ILE | F | 246 | 62.642 | 26.807 | 8.208 | 1.00 | 63.20 | F |
| ATOM | 20022 | CD1 | ILE | F | 246 | 61.487 | 26.468 | 9.130 | 1.00 | 63.20 | F |
| ATOM | 20023 | C | ILE | F | 246 | 62.576 | 23.822 | 5.777 | 1.00 | 77.06 | F |
| ATOM | 20024 | O | ILE | F | 246 | 63.550 | 23.943 | 5.032 | 1.00 | 77.06 | F |
| ATOM | 20025 | N | THR | F | 247 | 61.523 | 23.073 | 5.471 | 1.00 | 60.20 | F |
| ATOM | 20026 | CA | THR | F | 247 | 61.471 | 22.356 | 4.209 | 1.00 | 60.20 | F |
| ATOM | 20027 | CB | THR | F | 247 | 62.011 | 20.923 | 4.375 | 1.00 | 70.85 | F |
| ATOM | 20028 | OG1 | THR | F | 247 | 62.206 | 20.337 | 3.084 | 1.00 | 68.09 | F |
| ATOM | 20029 | CG2 | THR | F | 247 | 61.038 | 20.069 | 5.178 | 1.00 | 68.09 | F |
| ATOM | 20030 | C | THR | F | 247 | 60.033 | 22.320 | 3.692 | 1.00 | 60.20 | F |
| ATOM | 20031 | O | THR | F | 247 | 59.089 | 22.302 | 4.471 | 1.00 | 60.20 | F |
| ATOM | 20032 | N | GLY | F | 248 | 59.871 | 22.322 | 2.376 | 1.00 | 57.82 | F |
| ATOM | 20033 | CA | GLY | F | 248 | 58.539 | 22.304 | 1.799 | 1.00 | 57.82 | F |
| ATOM | 20034 | C | GLY | F | 248 | 58.575 | 22.655 | 0.322 | 1.00 | 57.82 | F |
| ATOM | 20035 | O | GLY | F | 248 | 59.650 | 22.754 | −0.273 | 1.00 | 57.82 | F |
| ATOM | 20036 | N | CYS | F | 249 | 57.411 | 22.848 | −0.283 | 1.00 | 67.95 | F |
| ATOM | 20037 | CA | CYS | F | 249 | 57.370 | 23.184 | −1.700 | 1.00 | 67.95 | F |
| ATOM | 20038 | CB | CYS | F | 249 | 57.220 | 21.921 | −2.547 | 1.00 | 49.94 | F |
| ATOM | 20039 | SG | CYS | F | 249 | 55.579 | 21.181 | −2.458 | 1.00 | 68.71 | F |
| ATOM | 20040 | C | CYS | F | 249 | 56.221 | 24.134 | −1.997 | 1.00 | 67.95 | F |
| ATOM | 20041 | O | CYS | F | 249 | 55.471 | 24.521 | −1.098 | 1.00 | 67.95 | F |
| ATOM | 20042 | N | TYR | F | 250 | 56.076 | 24.494 | −3.267 | 1.00 | 67.52 | F |
| ATOM | 20043 | CA | TYR | F | 250 | 55.027 | 25.416 | −3.674 | 1.00 | 67.52 | F |
| ATOM | 20044 | CB | TYR | F | 250 | 55.473 | 26.835 | −3.386 | 1.00 | 51.67 | F |
| ATOM | 20045 | CG | TYR | F | 250 | 56.700 | 27.221 | −4.175 | 1.00 | 42.02 | F |
| ATOM | 20046 | CD1 | TYR | F | 250 | 56.588 | 27.814 | −5.427 | 1.00 | 42.02 | F |
| ATOM | 20047 | CE1 | TYR | F | 250 | 57.716 | 28.167 | −6.157 | 1.00 | 42.02 | F |
| ATOM | 20048 | CD2 | TYR | F | 250 | 57.977 | 26.982 | −3.668 | 1.00 | 42.02 | F |
| ATOM | 20049 | CE2 | TYR | F | 250 | 59.114 | 27.324 | −4.384 | 1.00 | 42.02 | F |
| ATOM | 20050 | CZ | TYR | F | 250 | 58.979 | 27.915 | −5.624 | 1.00 | 42.02 | F |
| ATOM | 20051 | OH | TYR | F | 250 | 60.106 | 28.247 | −6.338 | 1.00 | 42.02 | F |
| ATOM | 20052 | C | TYR | F | 250 | 54.676 | 25.327 | −5.147 | 1.00 | 67.52 | F |
| ATOM | 20053 | O | TYR | F | 250 | 55.486 | 24.917 | −5.976 | 1.00 | 67.52 | F |
| ATOM | 20054 | N | GLU | F | 251 | 53.448 | 25.728 | −5.446 | 1.00 | 83.80 | F |
| ATOM | 20055 | CA | GLU | F | 251 | 52.927 | 25.786 | −6.798 | 1.00 | 83.80 | F |
| ATOM | 20056 | CB | GLU | F | 251 | 51.967 | 24.633 | −7.072 | 1.00 | 145.03 | F |
| ATOM | 20057 | CG | GLU | F | 251 | 52.502 | 23.662 | −8.106 | 1.00 | 98.65 | F |
| ATOM | 20058 | CD | GLU | F | 251 | 51.488 | 22.606 | −8.510 | 1.00 | 98.65 | F |
| ATOM | 20059 | OE1 | GLU | F | 251 | 50.374 | 22.978 | −8.949 | 1.00 | 98.65 | F |
| ATOM | 20060 | OE2 | GLU | F | 251 | 51.810 | 21.403 | −8.395 | 1.00 | 98.65 | F |
| ATOM | 20061 | C | GLU | F | 251 | 52.188 | 27.114 | −6.825 | 1.00 | 83.80 | F |
| ATOM | 20062 | O | GLU | F | 251 | 50.970 | 27.172 | −6.651 | 1.00 | 83.80 | F |
| ATOM | 20063 | N | THR | F | 252 | 52.957 | 28.184 | −7.000 | 1.00 | 58.37 | F |
| ATOM | 20064 | CA | THR | F | 252 | 52.432 | 29.551 | −7.042 | 1.00 | 58.37 | F |
| ATOM | 20065 | CB | THR | F | 252 | 53.439 | 30.537 | −6.402 | 1.00 | 82.21 | F |
| ATOM | 20066 | OG1 | THR | F | 252 | 53.788 | 30.084 | −5.090 | 1.00 | 82.21 | F |
| ATOM | 20067 | CG2 | THR | F | 252 | 52.838 | 31.918 | −6.298 | 1.00 | 82.21 | F |
| ATOM | 20068 | C | THR | F | 252 | 52.227 | 29.939 | −8.505 | 1.00 | 58.37 | F |
| ATOM | 20069 | O | THR | F | 252 | 53.165 | 30.375 | −9.174 | 1.00 | 58.37 | F |
| ATOM | 20070 | N | GLY | F | 253 | 51.003 | 29.787 | −9.000 | 1.00 | 70.39 | F |
| ATOM | 20071 | CA | GLY | F | 253 | 50.757 | 30.100 | −10.396 | 1.00 | 70.39 | F |
| ATOM | 20072 | C | GLY | F | 253 | 51.594 | 29.172 | −11.270 | 1.00 | 70.39 | F |
| ATOM | 20073 | O | GLY | F | 253 | 51.575 | 27.952 | −11.085 | 1.00 | 70.39 | F |
| ATOM | 20074 | N | LEU | F | 254 | 52.332 | 29.740 | −12.220 | 1.00 | 57.87 | F |
| ATOM | 20075 | CA | LEU | F | 254 | 53.180 | 28.940 | −13.092 | 1.00 | 57.87 | F |
| ATOM | 20076 | CB | LEU | F | 254 | 53.612 | 29.731 | −14.327 | 1.00 | 50.43 | F |
| ATOM | 20077 | CG | LEU | F | 254 | 52.569 | 30.083 | −15.376 | 1.00 | 50.43 | F |
| ATOM | 20078 | CD1 | LEU | F | 254 | 53.285 | 30.691 | −16.570 | 1.00 | 50.43 | F |
| ATOM | 20079 | CD2 | LEU | F | 254 | 51.780 | 28.844 | −15.772 | 1.00 | 50.43 | F |
| ATOM | 20080 | C | LEU | F | 254 | 54.433 | 28.482 | −12.372 | 1.00 | 57.87 | F |
| ATOM | 20081 | O | LEU | F | 254 | 54.985 | 27.444 | −12.695 | 1.00 | 57.87 | F |
| ATOM | 20082 | N | LEU | F | 255 | 54.893 | 29.268 | −11.411 | 1.00 | 42.70 | F |
| ATOM | 20083 | CA | LEU | F | 255 | 56.106 | 28.928 | −10.676 | 1.00 | 42.70 | F |
| ATOM | 20084 | CB | LEU | F | 255 | 56.596 | 30.129 | −9.860 | 1.00 | 57.87 | F |
| ATOM | 20085 | CG | LEU | F | 255 | 56.755 | 31.447 | −10.618 | 1.00 | 57.87 | F |
| ATOM | 20086 | CD1 | LEU | F | 255 | 57.311 | 32.502 | −9.679 | 1.00 | 57.87 | F |
| ATOM | 20087 | CD2 | LEU | F | 255 | 57.677 | 31.254 | −11.814 | 1.00 | 57.87 | F |
| ATOM | 20088 | C | LEU | F | 255 | 55.914 | 27.739 | −9.748 | 1.00 | 42.70 | F |
| ATOM | 20089 | O | LEU | F | 255 | 54.812 | 27.495 | −9.243 | 1.00 | 42.70 | F |
| ATOM | 20090 | N | PHE | F | 256 | 56.998 | 26.997 | −9.537 | 1.00 | 65.16 | F |
| ATOM | 20091 | CA | PHE | F | 256 | 56.967 | 25.831 | −8.662 | 1.00 | 65.16 | F |
| ATOM | 20092 | CB | PHE | F | 256 | 56.256 | 24.663 | −9.355 | 1.00 | 54.41 | F |
| ATOM | 20093 | CG | PHE | F | 256 | 56.988 | 24.123 | −10.546 | 1.00 | 51.42 | F |
| ATOM | 20094 | CD1 | PHE | F | 256 | 57.783 | 22.993 | −10.435 | 1.00 | 51.42 | F |
| ATOM | 20095 | CD2 | PHE | F | 256 | 56.876 | 24.739 | −11.792 | 1.00 | 51.42 | F |
| ATOM | 20096 | CE1 | PHE | F | 256 | 58.451 | 22.484 | −11.542 | 1.00 | 51.42 | F |
| ATOM | 20097 | CE2 | PHE | F | 256 | 57.545 | 24.231 | −12.909 | 1.00 | 51.42 | F |
| ATOM | 20098 | CZ | PHE | F | 256 | 58.331 | 23.103 | −12.778 | 1.00 | 51.42 | F |
| ATOM | 20099 | C | PHE | F | 256 | 58.380 | 25.435 | −8.254 | 1.00 | 65.16 | F |
| ATOM | 20100 | O | PHE | F | 256 | 59.318 | 25.566 | −9.040 | 1.00 | 65.16 | F |
| ATOM | 20101 | N | GLY | F | 257 | 58.530 | 24.971 | −7.016 | 1.00 | 50.51 | F |
| ATOM | 20102 | CA | GLY | F | 257 | 59.839 | 24.576 | −6.526 | 1.00 | 50.51 | F |
| ATOM | 20103 | C | GLY | F | 257 | 59.759 | 23.979 | −5.139 | 1.00 | 50.51 | F |
| ATOM | 20104 | O | GLY | F | 257 | 58.674 | 23.845 | −4.571 | 1.00 | 50.51 | F |
| ATOM | 20105 | N | SER | F | 258 | 60.906 | 23.599 | −4.593 | 1.00 | 48.05 | F |
| ATOM | 20106 | CA | SER | F | 258 | 60.951 | 23.021 | −3.254 | 1.00 | 48.05 | F |
| ATOM | 20107 | CB | SER | F | 258 | 61.029 | 21.497 | −3.327 | 1.00 | 53.19 | F |
| ATOM | 20108 | OG | SER | F | 258 | 62.267 | 21.091 | −3.879 | 1.00 | 63.31 | F |
| ATOM | 20109 | C | SER | F | 258 | 62.175 | 23.572 | −2.527 | 1.00 | 48.05 | F |
| ATOM | 20110 | O | SER | F | 258 | 63.098 | 24.099 | −3.147 | 1.00 | 48.05 | F |
| ATOM | 20111 | N | TYR | F | 259 | 62.188 | 23.448 | −1.209 | 1.00 | 49.73 | F |
| ATOM | 20112 | CA | TYR | F | 259 | 63.302 | 23.971 | −0.437 | 1.00 | 49.73 | F |
| ATOM | 20113 | CB | TYR | F | 259 | 63.030 | 25.432 | −0.068 | 1.00 | 70.86 | F |
| ATOM | 20114 | CG | TYR | F | 259 | 61.695 | 25.617 | 0.621 | 1.00 | 66.61 | F |
| ATOM | 20115 | CD1 | TYR | F | 259 | 61.540 | 25.339 | 1.983 | 1.00 | 66.61 | F |
| ATOM | 20116 | CE1 | TYR | F | 259 | 60.292 | 25.403 | 2.590 | 1.00 | 66.61 | F |
| ATOM | 20117 | CD2 | TYR | F | 259 | 60.562 | 25.975 | −0.110 | 1.00 | 66.61 | F |
| ATOM | 20118 | CE2 | TYR | F | 259 | 59.310 | 26.038 | 0.489 | 1.00 | 66.61 | F |
| ATOM | 20119 | CZ | TYR | F | 259 | 59.181 | 25.746 | 1.835 | 1.00 | 66.61 | F |
| ATOM | 20120 | OH | TYR | F | 259 | 57.928 | 25.753 | 2.401 | 1.00 | 66.61 | F |
| ATOM | 20121 | C | TYR | F | 259 | 63.536 | 23.161 | 0.826 | 1.00 | 49.73 | F |
| ATOM | 20122 | O | TYR | F | 259 | 62.667 | 22.401 | 1.284 | 1.00 | 49.73 | F |
| ATOM | 20123 | N | GLY | F | 260 | 64.729 | 23.338 | 1.384 | 1.00 | 64.95 | F |
| ATOM | 20124 | CA | GLY | F | 260 | 65.104 | 22.646 | 2.600 | 1.00 | 64.95 | F |
| ATOM | 20125 | C | GLY | F | 260 | 66.337 | 23.331 | 3.135 | 1.00 | 64.95 | F |
| ATOM | 20126 | O | GLY | F | 260 | 67.430 | 23.185 | 2.594 | 1.00 | 64.95 | F |
| ATOM | 20127 | N | TYR | F | 261 | 66.165 | 24.112 | 4.187 | 1.00 | 68.48 | F |
| ATOM | 20128 | CA | TYR | F | 261 | 67.295 | 24.810 | 4.759 | 1.00 | 68.48 | F |
| ATOM | 20129 | CB | TYR | F | 261 | 67.371 | 26.232 | 4.200 | 1.00 | 91.96 | F |
| ATOM | 20130 | CG | TYR | F | 261 | 66.160 | 27.089 | 4.492 | 1.00 | 75.75 | F |
| ATOM | 20131 | CD1 | TYR | F | 261 | 66.057 | 27.817 | 5.677 | 1.00 | 75.75 | F |
| ATOM | 20132 | CE1 | TYR | F | 261 | 64.962 | 28.642 | 5.926 | 1.00 | 75.75 | F |
| ATOM | 20133 | CD2 | TYR | F | 261 | 65.130 | 27.201 | 3.569 | 1.00 | 75.75 | F |
| ATOM | 20134 | CE2 | TYR | F | 261 | 64.029 | 28.022 | 3.811 | 1.00 | 75.75 | F |
| ATOM | 20135 | CZ | TYR | F | 261 | 63.956 | 28.741 | 4.987 | 1.00 | 75.75 | F |
| ATOM | 20136 | OH | TYR | F | 261 | 62.886 | 29.578 | 5.198 | 1.00 | 75.75 | F |
| ATOM | 20137 | C | TYR | F | 261 | 67.203 | 24.834 | 6.274 | 1.00 | 68.48 | F |
| ATOM | 20138 | O | TYR | F | 261 | 66.147 | 24.547 | 6.865 | 1.00 | 68.48 | F |
| ATOM | 20139 | N | VAL | F | 262 | 68.331 | 25.167 | 6.892 | 1.00 | 70.87 | F |
| ATOM | 20140 | CA | VAL | F | 262 | 68.440 | 25.241 | 8.340 | 1.00 | 70.87 | F |
| ATOM | 20141 | CB | VAL | F | 262 | 69.593 | 24.345 | 8.852 | 1.00 | 80.86 | F |
| ATOM | 20142 | CG1 | VAL | F | 262 | 69.857 | 24.622 | 10.319 | 1.00 | 72.35 | F |
| ATOM | 20143 | CG2 | VAL | F | 262 | 69.243 | 22.876 | 8.647 | 1.00 | 72.35 | F |
| ATOM | 20144 | C | VAL | F | 262 | 68.729 | 26.667 | 8.762 | 1.00 | 70.87 | F |
| ATOM | 20145 | O | VAL | F | 262 | 69.716 | 27.253 | 8.322 | 1.00 | 70.87 | F |
| ATOM | 20146 | N | GLU | F | 263 | 67.865 | 27.231 | 9.601 | 1.00 | 82.87 | F |
| ATOM | 20147 | CA | GLU | F | 263 | 68.084 | 28.582 | 10.093 | 1.00 | 82.87 | F |
| ATOM | 20148 | CB | GLU | F | 263 | 66.952 | 29.527 | 9.679 | 1.00 | 115.19 | F |
| ATOM | 20149 | CG | GLU | F | 263 | 65.562 | 29.068 | 10.056 | 1.00 | 101.79 | F |
| ATOM | 20150 | CD | GLU | F | 263 | 64.500 | 30.067 | 9.639 | 1.00 | 101.79 | F |
| ATOM | 20151 | OE1 | GLU | F | 263 | 64.615 | 30.631 | 8.523 | 1.00 | 101.79 | F |
| ATOM | 20152 | OE2 | GLU | F | 263 | 63.547 | 30.280 | 10.421 | 1.00 | 101.79 | F |
| ATOM | 20153 | C | GLU | F | 263 | 68.242 | 28.573 | 11.606 | 1.00 | 82.87 | F |
| ATOM | 20154 | O | GLU | F | 263 | 67.831 | 27.635 | 12.305 | 1.00 | 82.87 | F |
| ATOM | 20155 | N | THR | F | 264 | 68.851 | 29.642 | 12.094 | 1.00 | 139.63 | F |
| ATOM | 20156 | CA | THR | F | 264 | 69.137 | 29.821 | 13.502 | 1.00 | 139.63 | F |
| ATOM | 20157 | CB | THR | F | 264 | 70.664 | 29.997 | 13.669 | 1.00 | 104.34 | F |
| ATOM | 20158 | OG1 | THR | F | 264 | 71.237 | 28.754 | 14.084 | 1.00 | 83.27 | F |
| ATOM | 20159 | CG2 | THR | F | 264 | 71.002 | 31.084 | 14.651 | 1.00 | 83.27 | F |
| ATOM | 20160 | C | THR | F | 264 | 68.385 | 31.042 | 14.009 | 1.00 | 139.63 | F |
| ATOM | 20161 | O | THR | F | 264 | 67.857 | 31.817 | 13.217 | 1.00 | 139.63 | F |
| ATOM | 20162 | N | ALA | F | 265 | 68.314 | 31.207 | 15.324 | 1.00 | 108.16 | F |
| ATOM | 20163 | CA | ALA | F | 265 | 67.632 | 32.365 | 15.884 | 1.00 | 108.16 | F |
| ATOM | 20164 | CB | ALA | F | 265 | 67.657 | 32.311 | 17.410 | 1.00 | 101.10 | F |
| ATOM | 20165 | C | ALA | F | 265 | 68.346 | 33.620 | 15.382 | 1.00 | 108.16 | F |
| ATOM | 20166 | O | ALA | F | 265 | 67.765 | 34.704 | 15.342 | 1.00 | 108.16 | F |
| ATOM | 20167 | N | ASN | F | 266 | 69.605 | 33.452 | 14.984 | 1.00 | 100.59 | F |
| ATOM | 20168 | CA | ASN | F | 266 | 70.420 | 34.553 | 14.479 | 1.00 | 100.59 | F |
| ATOM | 20169 | CB | ASN | F | 266 | 71.903 | 34.284 | 14.735 | 1.00 | 158.50 | F |
| ATOM | 20170 | CG | ASN | F | 266 | 72.197 | 34.004 | 16.186 | 1.00 | 100.35 | F |
| ATOM | 20171 | OD1 | ASN | F | 266 | 71.815 | 34.772 | 17.067 | 1.00 | 100.35 | F |
| ATOM | 20172 | ND2 | ASN | F | 266 | 72.884 | 32.900 | 16.448 | 1.00 | 100.35 | F |
| ATOM | 20173 | C | ASN | F | 266 | 70.215 | 34.791 | 12.991 | 1.00 | 100.59 | F |
| ATOM | 20174 | O | ASN | F | 266 | 70.633 | 35.821 | 12.463 | 1.00 | 100.59 | F |
| ATOM | 20175 | N | GLY | F | 267 | 69.583 | 33.835 | 12.315 | 1.00 | 129.75 | F |
| ATOM | 20176 | CA | GLY | F | 267 | 69.336 | 33.984 | 10.891 | 1.00 | 129.75 | F |
| ATOM | 20177 | C | GLY | F | 267 | 69.667 | 32.753 | 10.069 | 1.00 | 129.75 | F |
| ATOM | 20178 | O | GLY | F | 267 | 70.205 | 31.779 | 10.595 | 1.00 | 129.75 | F |
| ATOM | 20179 | N | PRO | F | 268 | 69.366 | 32.776 | 8.760 | 1.00 | 85.73 | F |
| ATOM | 20180 | CD | PRO | F | 268 | 68.973 | 33.963 | 7.981 | 1.00 | 106.67 | F |
| ATOM | 20181 | CA | PRO | F | 268 | 69.638 | 31.646 | 7.869 | 1.00 | 85.73 | F |
| ATOM | 20182 | CB | PRO | F | 268 | 69.366 | 32.225 | 6.480 | 1.00 | 122.56 | F |
| ATOM | 20183 | CG | PRO | F | 268 | 69.628 | 33.688 | 6.655 | 1.00 | 106.67 | F |
| ATOM | 20184 | C | PRO | F | 268 | 71.055 | 31.105 | 8.009 | 1.00 | 85.73 | F |
| ATOM | 20185 | O | PRO | F | 268 | 72.021 | 31.861 | 8.068 | 1.00 | 85.73 | F |
| ATOM | 20186 | N | LEU | F | 269 | 71.163 | 29.784 | 8.050 | 1.00 | 77.59 | F |
| ATOM | 20187 | CA | LEU | F | 269 | 72.448 | 29.125 | 8.203 | 1.00 | 77.59 | F |
| ATOM | 20188 | CB | LEU | F | 269 | 72.370 | 28.144 | 9.376 | 1.00 | 115.80 | F |
| ATOM | 20189 | CG | LEU | F | 269 | 73.686 | 27.642 | 9.968 | 1.00 | 81.14 | F |
| ATOM | 20190 | CD1 | LEU | F | 269 | 74.567 | 28.825 | 10.343 | 1.00 | 81.14 | F |
| ATOM | 20191 | CD2 | LEU | F | 269 | 73.388 | 26.780 | 11.186 | 1.00 | 81.14 | F |
| ATOM | 20192 | C | LEU | F | 269 | 72.910 | 28.399 | 6.937 | 1.00 | 77.59 | F |
| ATOM | 20193 | O | LEU | F | 269 | 73.823 | 28.867 | 6.250 | 1.00 | 77.59 | F |
| ATOM | 20194 | N | ALA | F | 270 | 72.283 | 27.262 | 6.631 | 1.00 | 75.71 | F |
| ATOM | 20195 | CA | ALA | F | 270 | 72.651 | 26.481 | 5.448 | 1.00 | 75.71 | F |
| ATOM | 20196 | CB | ALA | F | 270 | 73.562 | 25.332 | 5.844 | 1.00 | 80.19 | F |
| ATOM | 20197 | C | ALA | F | 270 | 71.463 | 25.939 | 4.666 | 1.00 | 75.71 | F |
| ATOM | 20198 | O | ALA | F | 270 | 70.441 | 25.543 | 5.231 | 1.00 | 75.71 | F |
| ATOM | 20199 | N | ARG | F | 271 | 71.636 | 25.897 | 3.351 | 1.00 | 69.06 | F |
| ATOM | 20200 | CA | ARG | F | 271 | 70.611 | 25.431 | 2.426 | 1.00 | 69.06 | F |
| ATOM | 20201 | CB | ARG | F | 271 | 70.437 | 26.496 | 1.343 | 1.00 | 67.86 | F |
| ATOM | 20202 | CG | ARG | F | 271 | 69.611 | 26.106 | 0.160 | 1.00 | 60.98 | F |
| ATOM | 20203 | CD | ARG | F | 271 | 69.627 | 27.224 | −0.868 | 1.00 | 60.98 | F |
| ATOM | 20204 | NE | ARG | F | 271 | 68.879 | 28.398 | −0.427 | 1.00 | 60.98 | F |
| ATOM | 20205 | CZ | ARG | F | 271 | 69.430 | 29.547 | −0.045 | 1.00 | 60.98 | F |
| ATOM | 20206 | NH1 | ARG | F | 271 | 70.751 | 29.695 | −0.041 | 1.00 | 60.98 | F |
| ATOM | 20207 | NH2 | ARG | F | 271 | 68.652 | 30.554 | 0.326 | 1.00 | 60.98 | F |
| ATOM | 20208 | C | ARG | F | 271 | 71.040 | 24.092 | 1.822 | 1.00 | 69.06 | F |
| ATOM | 20209 | O | ARG | F | 271 | 72.202 | 23.916 | 1.450 | 1.00 | 69.06 | F |
| ATOM | 20210 | N | TYR | F | 272 | 70.108 | 23.146 | 1.736 | 1.00 | 62.68 | F |
| ATOM | 20211 | CA | TYR | F | 272 | 70.414 | 21.824 | 1.183 | 1.00 | 62.68 | F |
| ATOM | 20212 | CB | TYR | F | 272 | 69.580 | 20.743 | 1.866 | 1.00 | 71.46 | F |
| ATOM | 20213 | CG | TYR | F | 272 | 69.906 | 19.358 | 1.364 | 1.00 | 68.93 | F |
| ATOM | 20214 | CD1 | TYR | F | 272 | 71.010 | 18.664 | 1.858 | 1.00 | 68.93 | F |
| ATOM | 20215 | CE1 | TYR | F | 272 | 71.338 | 17.390 | 1.387 | 1.00 | 68.93 | F |
| ATOM | 20216 | CD2 | TYR | F | 272 | 69.131 | 18.746 | 0.378 | 1.00 | 68.93 | F |
| ATOM | 20217 | CE2 | TYR | F | 272 | 69.447 | 17.474 | −0.103 | 1.00 | 68.93 | F |
| ATOM | 20218 | CZ | TYR | F | 272 | 70.555 | 16.799 | 0.409 | 1.00 | 68.93 | F |
| ATOM | 20219 | OH | TYR | F | 272 | 70.890 | 15.536 | −0.035 | 1.00 | 68.93 | F |
| ATOM | 20220 | C | TYR | F | 272 | 70.155 | 21.743 | −0.318 | 1.00 | 62.68 | F |
| ATOM | 20221 | O | TYR | F | 272 | 69.021 | 21.945 | −0.769 | 1.00 | 62.68 | F |
| ATOM | 20222 | N | HIS | F | 273 | 71.198 | 21.431 | −1.087 | 1.00 | 75.65 | F |
| ATOM | 20223 | CA | HIS | F | 273 | 71.058 | 21.336 | −2.536 | 1.00 | 75.65 | F |
| ATOM | 20224 | CB | HIS | F | 273 | 72.234 | 22.011 | −3.241 | 1.00 | 180.54 | F |
| ATOM | 20225 | CG | HIS | F | 273 | 72.171 | 23.506 | −3.204 | 1.00 | 161.27 | F |
| ATOM | 20226 | CD2 | HIS | F | 273 | 73.017 | 24.424 | −2.681 | 1.00 | 161.27 | F |
| ATOM | 20227 | ND1 | HIS | F | 273 | 71.112 | 24.218 | −3.728 | 1.00 | 161.27 | F |
| ATOM | 20228 | CE1 | HIS | F | 273 | 71.309 | 25.509 | −3.528 | 1.00 | 161.27 | F |
| ATOM | 20229 | NE2 | HIS | F | 273 | 72.458 | 25.661 | −2.895 | 1.00 | 161.27 | F |
| ATOM | 20230 | C | HIS | F | 273 | 70.894 | 19.914 | −3.029 | 1.00 | 75.65 | F |
| ATOM | 20231 | O | HIS | F | 273 | 71.757 | 19.063 | −2.833 | 1.00 | 75.65 | F |
| ATOM | 20232 | N | THR | F | 274 | 69.751 | 19.677 | −3.662 | 1.00 | 67.63 | F |
| ATOM | 20233 | CA | THR | F | 274 | 69.399 | 18.383 | −4.213 | 1.00 | 67.63 | F |
| ATOM | 20234 | CB | THR | F | 274 | 68.028 | 18.481 | −4.908 | 1.00 | 87.34 | F |
| ATOM | 20235 | OG1 | THR | F | 274 | 67.009 | 18.053 | −3.996 | 1.00 | 79.87 | F |
| ATOM | 20236 | CG2 | THR | F | 274 | 67.996 | 17.649 | −6.188 | 1.00 | 79.87 | F |
| ATOM | 20237 | C | THR | F | 274 | 70.454 | 17.866 | −5.196 | 1.00 | 67.63 | F |
| ATOM | 20238 | O | THR | F | 274 | 70.726 | 16.662 | −5.260 | 1.00 | 67.63 | F |
| ATOM | 20239 | N | GLY | F | 275 | 71.040 | 18.784 | −5.958 | 1.00 | 66.44 | F |
| ATOM | 20240 | CA | GLY | F | 275 | 72.050 | 18.405 | −6.926 | 1.00 | 66.44 | F |
| ATOM | 20241 | C | GLY | F | 275 | 73.329 | 17.898 | −6.292 | 1.00 | 66.44 | F |
| ATOM | 20242 | O | GLY | F | 275 | 73.683 | 16.736 | −6.459 | 1.00 | 66.44 | F |
| ATOM | 20243 | N | ASP | F | 276 | 74.014 | 18.769 | −5.555 | 1.00 | 67.53 | F |
| ATOM | 20244 | CA | ASP | F | 276 | 75.275 | 18.435 | −4.892 | 1.00 | 67.53 | F |
| ATOM | 20245 | CB | ASP | F | 276 | 75.918 | 19.711 | −4.354 | 1.00 | 177.74 | F |
| ATOM | 20246 | CG | ASP | F | 276 | 75.976 | 20.808 | −5.389 | 1.00 | 156.74 | F |
| ATOM | 20247 | OD1 | ASP | F | 276 | 76.740 | 20.662 | −6.368 | 1.00 | 156.74 | F |
| ATOM | 20248 | OD2 | ASP | F | 276 | 75.249 | 21.811 | −5.227 | 1.00 | 156.74 | F |
| ATOM | 20249 | C | ASP | F | 276 | 75.134 | 17.434 | −3.746 | 1.00 | 67.53 | F |
| ATOM | 20250 | O | ASP | F | 276 | 76.119 | 16.827 | −3.331 | 1.00 | 67.53 | F |
| ATOM | 20251 | N | LYS | F | 277 | 73.916 | 17.264 | −3.235 | 1.00 | 70.31 | F |
| ATOM | 20252 | CA | LYS | F | 277 | 73.666 | 16.354 | −2.120 | 1.00 | 70.31 | F |
| ATOM | 20253 | CB | LYS | F | 277 | 73.975 | 14.918 | −2.533 | 1.00 | 89.10 | F |
| ATOM | 20254 | CG | LYS | F | 277 | 73.026 | 14.375 | −3.587 | 1.00 | 74.31 | F |
| ATOM | 20255 | CD | LYS | F | 277 | 73.563 | 13.093 | −4.202 | 1.00 | 74.31 | F |
| ATOM | 20256 | CE | LYS | F | 277 | 72.597 | 12.483 | −5.211 | 1.00 | 74.31 | F |
| ATOM | 20257 | NZ | LYS | F | 277 | 71.428 | 11.825 | −4.555 | 1.00 | 74.31 | F |
| ATOM | 20258 | C | LYS | F | 277 | 74.500 | 16.756 | −0.902 | 1.00 | 70.31 | F |
| ATOM | 20259 | O | LYS | F | 277 | 75.187 | 15.934 | −0.300 | 1.00 | 70.31 | F |
| ATOM | 20260 | N | ARG | F | 278 | 74.420 | 18.036 | −0.552 | 1.00 | 77.13 | F |
| ATOM | 20261 | CA | ARG | F | 278 | 75.145 | 18.586 | 0.580 | 1.00 | 77.13 | F |
| ATOM | 20262 | CB | ARG | F | 278 | 76.611 | 18.727 | 0.219 | 1.00 | 117.71 | F |
| ATOM | 20263 | CG | ARG | F | 278 | 76.860 | 19.835 | −0.779 | 1.00 | 102.34 | F |
| ATOM | 20264 | CD | ARG | F | 278 | 78.258 | 19.783 | −1.333 | 1.00 | 102.34 | F |
| ATOM | 20265 | NE | ARG | F | 278 | 78.568 | 20.912 | −2.195 | 1.00 | 102.34 | F |
| ATOM | 20266 | CZ | ARG | F | 278 | 79.626 | 20.970 | −2.998 | 1.00 | 102.34 | F |
| ATOM | 20267 | NH1 | ARG | F | 278 | 80.486 | 19.958 | −3.052 | 1.00 | 102.34 | F |
| ATOM | 20268 | NH2 | ARG | F | 278 | 79.817 | 22.039 | −3.763 | 1.00 | 102.34 | F |
| ATOM | 20269 | C | ARG | F | 278 | 74.572 | 19.967 | 0.911 | 1.00 | 77.13 | F |
| ATOM | 20270 | O | ARG | F | 278 | 73.798 | 20.521 | 0.130 | 1.00 | 77.13 | F |
| ATOM | 20271 | N | PHE | F | 279 | 74.949 | 20.517 | 2.065 | 1.00 | 69.16 | F |
| ATOM | 20272 | CA | PHE | F | 279 | 74.473 | 21.837 | 2.457 | 1.00 | 69.16 | F |
| ATOM | 20273 | CB | PHE | F | 279 | 74.409 | 21.975 | 3.972 | 1.00 | 73.45 | F |
| ATOM | 20274 | CG | PHE | F | 279 | 73.206 | 21.347 | 4.581 | 1.00 | 57.22 | F |
| ATOM | 20275 | CD1 | PHE | F | 279 | 73.244 | 20.043 | 5.055 | 1.00 | 57.22 | F |
| ATOM | 20276 | CD2 | PHE | F | 279 | 72.026 | 22.067 | 4.694 | 1.00 | 57.22 | F |
| ATOM | 20277 | CE1 | PHE | F | 279 | 72.115 | 19.463 | 5.642 | 1.00 | 57.22 | F |
| ATOM | 20278 | CE2 | PHE | F | 279 | 70.888 | 21.497 | 5.278 | 1.00 | 57.22 | F |
| ATOM | 20279 | CZ | PHE | F | 279 | 70.933 | 20.190 | 5.755 | 1.00 | 57.22 | F |
| ATOM | 20280 | C | PHE | F | 279 | 75.398 | 22.905 | 1.915 | 1.00 | 69.16 | F |
| ATOM | 20281 | O | PHE | F | 279 | 76.584 | 22.650 | 1.681 | 1.00 | 69.16 | F |
| ATOM | 20282 | N | THR | F | 280 | 74.851 | 24.102 | 1.719 | 1.00 | 73.18 | F |
| ATOM | 20283 | CA | THR | F | 280 | 75.628 | 25.224 | 1.217 | 1.00 | 73.18 | F |
| ATOM | 20284 | CB | THR | F | 280 | 75.319 | 25.515 | −0.254 | 1.00 | 113.40 | F |
| ATOM | 20285 | OG1 | THR | F | 280 | 75.423 | 24.305 | −1.011 | 1.00 | 93.92 | F |
| ATOM | 20286 | CG2 | THR | F | 280 | 76.305 | 26.539 | −0.805 | 1.00 | 93.92 | F |
| ATOM | 20287 | C | THR | F | 280 | 75.294 | 26.467 | 2.016 | 1.00 | 73.18 | F |
| ATOM | 20288 | O | THR | F | 280 | 74.128 | 26.711 | 2.338 | 1.00 | 73.18 | F |
| ATOM | 20289 | N | LYS | F | 281 | 76.322 | 27.249 | 2.330 | 1.00 | 84.83 | F |
| ATOM | 20290 | CA | LYS | F | 281 | 76.151 | 28.486 | 3.083 | 1.00 | 84.83 | F |
| ATOM | 20291 | CB | LYS | F | 281 | 77.479 | 29.247 | 3.112 | 1.00 | 142.00 | F |
| ATOM | 20292 | CG | LYS | F | 281 | 78.264 | 29.135 | 1.809 | 1.00 | 117.94 | F |
| ATOM | 20293 | CD | LYS | F | 281 | 78.728 | 30.494 | 1.279 | 1.00 | 117.94 | F |
| ATOM | 20294 | CE | LYS | F | 281 | 79.754 | 31.156 | 2.187 | 1.00 | 117.94 | F |
| ATOM | 20295 | NZ | LYS | F | 281 | 80.240 | 32.451 | 1.626 | 1.00 | 117.94 | F |
| ATOM | 20296 | C | LYS | F | 281 | 75.056 | 29.380 | 2.486 | 1.00 | 84.83 | F |
| ATOM | 20297 | O | LYS | F | 281 | 74.919 | 29.476 | 1.271 | 1.00 | 84.83 | F |
| ATOM | 20298 | N | MET | F | 282 | 74.267 | 30.016 | 3.347 | 1.00 | 81.96 | F |
| ATOM | 20299 | CA | MET | F | 282 | 73.220 | 30.925 | 2.897 | 1.00 | 81.96 | F |
| ATOM | 20300 | CB | MET | F | 282 | 71.921 | 30.682 | 3.654 | 1.00 | 81.01 | F |
| ATOM | 20301 | CG | MET | F | 282 | 71.233 | 29.395 | 3.315 | 1.00 | 81.01 | F |
| ATOM | 20302 | SD | MET | F | 282 | 69.515 | 29.428 | 3.865 | 1.00 | 81.01 | F |
| ATOM | 20303 | CE | MET | F | 282 | 69.675 | 28.756 | 5.491 | 1.00 | 81.01 | F |
| ATOM | 20304 | C | MET | F | 282 | 73.671 | 32.360 | 3.157 | 1.00 | 81.96 | F |
| ATOM | 20305 | O | MET | F | 282 | 73.023 | 33.314 | 2.730 | 1.00 | 81.96 | F |
| ATOM | 20306 | N | THR | F | 283 | 74.785 | 32.495 | 3.872 | 1.00 | 94.18 | F |
| ATOM | 20307 | CA | THR | F | 283 | 75.351 | 33.790 | 4.225 | 1.00 | 94.18 | F |
| ATOM | 20308 | CB | THR | F | 283 | 75.086 | 34.112 | 5.708 | 1.00 | 93.42 | F |
| ATOM | 20309 | OG1 | THR | F | 283 | 73.678 | 34.241 | 5.922 | 1.00 | 93.42 | F |
| ATOM | 20310 | CG2 | THR | F | 283 | 75.769 | 35.396 | 6.113 | 1.00 | 93.42 | F |
| ATOM | 20311 | C | THR | F | 283 | 76.853 | 33.739 | 4.006 | 1.00 | 94.18 | F |
| ATOM | 20312 | O | THR | F | 283 | 77.435 | 32.658 | 3.960 | 1.00 | 94.18 | F |
| ATOM | 20313 | N | HIS | F | 284 | 77.477 | 34.905 | 3.861 | 1.00 | 130.18 | F |
| ATOM | 20314 | CA | HIS | F | 284 | 78.925 | 34.995 | 3.671 | 1.00 | 130.18 | F |
| ATOM | 20315 | CB | HIS | F | 284 | 79.344 | 36.467 | 3.563 | 1.00 | 161.88 | F |
| ATOM | 20316 | CG | HIS | F | 284 | 78.921 | 37.304 | 4.735 | 1.00 | 161.88 | F |
| ATOM | 20317 | CD2 | HIS | F | 284 | 79.646 | 37.950 | 5.680 | 1.00 | 161.88 | F |
| ATOM | 20318 | ND1 | HIS | F | 284 | 77.597 | 37.526 | 5.052 | 1.00 | 161.88 | F |
| ATOM | 20319 | CE1 | HIS | F | 284 | 77.525 | 38.271 | 6.142 | 1.00 | 161.88 | F |
| ATOM | 20320 | NE2 | HIS | F | 284 | 78.754 | 38.542 | 6.542 | 1.00 | 161.88 | F |
| ATOM | 20321 | C | HIS | F | 284 | 79.612 | 34.353 | 4.880 | 1.00 | 130.18 | F |
| ATOM | 20322 | O | HIS | F | 284 | 80.653 | 33.705 | 4.757 | 1.00 | 130.18 | F |
| ATOM | 20323 | N | LYS | F | 285 | 78.991 | 34.540 | 6.042 | 1.00 | 116.59 | F |
| ATOM | 20324 | CA | LYS | F | 285 | 79.476 | 34.034 | 7.319 | 1.00 | 116.59 | F |
| ATOM | 20325 | CB | LYS | F | 285 | 78.417 | 34.278 | 8.399 | 1.00 | 106.97 | F |
| ATOM | 20326 | CG | LYS | F | 285 | 78.939 | 34.969 | 9.652 | 1.00 | 106.97 | F |
| ATOM | 20327 | CD | LYS | F | 285 | 79.422 | 36.384 | 9.347 | 1.00 | 106.97 | F |
| ATOM | 20328 | CE | LYS | F | 285 | 79.932 | 37.081 | 10.598 | 1.00 | 106.97 | F |
| ATOM | 20329 | NZ | LYS | F | 285 | 80.383 | 38.471 | 10.314 | 1.00 | 106.97 | F |
| ATOM | 20330 | C | LYS | F | 285 | 79.849 | 32.556 | 7.315 | 1.00 | 116.59 | F |
| ATOM | 20331 | O | LYS | F | 285 | 80.752 | 32.142 | 8.041 | 1.00 | 116.59 | F |
| ATOM | 20332 | N | GLY | F | 286 | 79.154 | 31.763 | 6.507 | 1.00 | 96.53 | F |
| ATOM | 20333 | CA | GLY | F | 286 | 79.425 | 30.335 | 6.455 | 1.00 | 96.53 | F |
| ATOM | 20334 | C | GLY | F | 286 | 78.630 | 29.616 | 7.534 | 1.00 | 96.53 | F |
| ATOM | 20335 | O | GLY | F | 286 | 77.835 | 30.239 | 8.242 | 1.00 | 96.53 | F |
| ATOM | 20336 | N | PHE | F | 287 | 78.829 | 28.308 | 7.664 | 1.00 | 83.86 | F |
| ATOM | 20337 | CA | PHE | F | 287 | 78.117 | 27.542 | 8.685 | 1.00 | 83.86 | F |
| ATOM | 20338 | CB | PHE | F | 287 | 76.833 | 26.924 | 8.106 | 1.00 | 78.92 | F |
| ATOM | 20339 | CG | PHE | F | 287 | 77.075 | 25.816 | 7.100 | 1.00 | 78.92 | F |
| ATOM | 20340 | CD1 | PHE | F | 287 | 77.276 | 24.499 | 7.516 | 1.00 | 78.92 | F |
| ATOM | 20341 | CD2 | PHE | F | 287 | 77.113 | 26.095 | 5.733 | 1.00 | 78.92 | F |
| ATOM | 20342 | CE1 | PHE | F | 287 | 77.513 | 23.480 | 6.587 | 1.00 | 78.92 | F |
| ATOM | 20343 | CE2 | PHE | F | 287 | 77.351 | 25.076 | 4.796 | 1.00 | 78.92 | F |
| ATOM | 20344 | CZ | PHE | F | 287 | 77.551 | 23.771 | 5.227 | 1.00 | 78.92 | F |
| ATOM | 20345 | C | PHE | F | 287 | 79.005 | 26.449 | 9.268 | 1.00 | 83.86 | F |
| ATOM | 20346 | O | PHE | F | 287 | 79.791 | 25.821 | 8.554 | 1.00 | 83.86 | F |
| ATOM | 20347 | N | PRO | F | 288 | 78.889 | 26.209 | 10.583 | 1.00 | 84.99 | F |
| ATOM | 20348 | CD | PRO | F | 288 | 77.947 | 26.853 | 11.517 | 1.00 | 77.17 | F |
| ATOM | 20349 | CA | PRO | F | 288 | 79.684 | 25.187 | 11.268 | 1.00 | 84.99 | F |
| ATOM | 20350 | CB | PRO | F | 288 | 79.338 | 25.416 | 12.738 | 1.00 | 77.17 | F |
| ATOM | 20351 | CG | PRO | F | 288 | 77.914 | 25.871 | 12.672 | 1.00 | 77.17 | F |
| ATOM | 20352 | C | PRO | F | 288 | 79.353 | 23.772 | 10.803 | 1.00 | 84.99 | F |
| ATOM | 20353 | O | PRO | F | 288 | 78.200 | 23.343 | 10.861 | 1.00 | 84.99 | F |
| ATOM | 20354 | N | LYS | F | 289 | 80.367 | 23.050 | 10.336 | 1.00 | 99.14 | F |
| ATOM | 20355 | CA | LYS | F | 289 | 80.159 | 21.685 | 9.875 | 1.00 | 99.14 | F |
| ATOM | 20356 | CB | LYS | F | 289 | 81.255 | 21.291 | 8.880 | 1.00 | 123.38 | F |
| ATOM | 20357 | CG | LYS | F | 289 | 81.037 | 21.900 | 7.499 | 1.00 | 123.38 | F |
| ATOM | 20358 | CD | LYS | F | 289 | 82.130 | 21.521 | 6.520 | 1.00 | 123.38 | F |
| ATOM | 20359 | CE | LYS | F | 289 | 83.440 | 22.211 | 6.868 | 1.00 | 123.38 | F |
| ATOM | 20360 | NZ | LYS | F | 289 | 84.529 | 21.886 | 5.897 | 1.00 | 123.38 | F |
| ATOM | 20361 | C | LYS | F | 289 | 80.094 | 20.709 | 11.048 | 1.00 | 99.14 | F |
| ATOM | 20362 | O | LYS | F | 289 | 81.049 | 19.990 | 11.346 | 1.00 | 99.14 | F |
| ATOM | 20363 | N | VAL | F | 290 | 78.934 | 20.696 | 11.700 | 1.00 | 100.07 | F |
| ATOM | 20364 | CA | VAL | F | 290 | 78.679 | 19.841 | 12.850 | 1.00 | 100.07 | F |
| ATOM | 20365 | CB | VAL | F | 290 | 78.762 | 20.653 | 14.152 | 1.00 | 88.08 | F |
| ATOM | 20366 | CG1 | VAL | F | 290 | 80.063 | 21.425 | 14.191 | 1.00 | 88.08 | F |
| ATOM | 20367 | CG2 | VAL | F | 290 | 77.578 | 21.613 | 14.247 | 1.00 | 88.08 | F |
| ATOM | 20368 | C | VAL | F | 290 | 77.277 | 19.243 | 12.748 | 1.00 | 100.07 | F |
| ATOM | 20369 | O | VAL | F | 290 | 76.473 | 19.671 | 11.921 | 1.00 | 100.07 | F |
| ATOM | 20370 | N | GLY | F | 291 | 76.992 | 18.263 | 13.601 | 1.00 | 82.21 | F |
| ATOM | 20371 | CA | GLY | F | 291 | 75.685 | 17.623 | 13.610 | 1.00 | 82.21 | F |
| ATOM | 20372 | C | GLY | F | 291 | 75.143 | 17.183 | 12.260 | 1.00 | 82.21 | F |
| ATOM | 20373 | O | GLY | F | 291 | 75.838 | 16.551 | 11.462 | 1.00 | 82.21 | F |
| ATOM | 20374 | N | MET | F | 292 | 73.884 | 17.519 | 12.005 | 1.00 | 65.27 | F |
| ATOM | 20375 | CA | MET | F | 292 | 73.221 | 17.171 | 10.754 | 1.00 | 65.27 | F |
| ATOM | 20376 | CB | MET | F | 292 | 71.740 | 17.481 | 10.861 | 1.00 | 81.53 | F |
| ATOM | 20377 | CG | MET | F | 292 | 71.501 | 18.972 | 10.915 | 1.00 | 81.53 | F |
| ATOM | 20378 | SD | MET | F | 292 | 69.833 | 19.430 | 10.526 | 1.00 | 81.53 | F |
| ATOM | 20379 | CE | MET | F | 292 | 69.283 | 20.070 | 12.103 | 1.00 | 81.53 | F |
| ATOM | 20380 | C | MET | F | 292 | 73.766 | 17.950 | 9.558 | 1.00 | 65.27 | F |
| ATOM | 20381 | O | MET | F | 292 | 73.307 | 17.759 | 8.441 | 1.00 | 65.27 | F |
| ATOM | 20382 | N | LEU | F | 293 | 74.733 | 18.827 | 9.788 | 1.00 | 89.08 | F |
| ATOM | 20383 | CA | LEU | F | 293 | 75.273 | 19.639 | 8.708 | 1.00 | 89.08 | F |
| ATOM | 20384 | CB | LEU | F | 293 | 75.530 | 21.058 | 9.220 | 1.00 | 81.22 | F |
| ATOM | 20385 | CG | LEU | F | 293 | 74.285 | 21.834 | 9.660 | 1.00 | 81.22 | F |
| ATOM | 20386 | CD1 | LEU | F | 293 | 74.686 | 23.170 | 10.285 | 1.00 | 81.22 | F |
| ATOM | 20387 | CD2 | LEU | F | 293 | 73.382 | 22.041 | 8.455 | 1.00 | 81.22 | F |
| ATOM | 20388 | C | LEU | F | 293 | 76.518 | 19.131 | 7.987 | 1.00 | 89.08 | F |
| ATOM | 20389 | O | LEU | F | 293 | 76.957 | 19.759 | 7.026 | 1.00 | 89.08 | F |
| ATOM | 20390 | N | THR | F | 294 | 77.096 | 18.013 | 8.428 | 1.00 | 71.68 | F |
| ATOM | 20391 | CA | THR | F | 294 | 78.293 | 17.487 | 7.760 | 1.00 | 71.68 | F |
| ATOM | 20392 | CB | THR | F | 294 | 79.549 | 17.520 | 8.639 | 1.00 | 112.61 | F |
| ATOM | 20393 | OG1 | THR | F | 294 | 79.574 | 18.727 | 9.404 | 1.00 | 112.61 | F |
| ATOM | 20394 | CG2 | THR | F | 294 | 80.797 | 17.462 | 7.756 | 1.00 | 112.61 | F |
| ATOM | 20395 | C | THR | F | 294 | 78.142 | 16.049 | 7.332 | 1.00 | 71.68 | F |
| ATOM | 20396 | O | THR | F | 294 | 77.430 | 15.265 | 7.955 | 1.00 | 71.68 | F |
| ATOM | 20397 | N | VAL | F | 295 | 78.843 | 15.702 | 6.266 | 1.00 | 62.88 | F |
| ATOM | 20398 | CA | VAL | F | 295 | 78.798 | 14.350 | 5.755 | 1.00 | 62.88 | F |
| ATOM | 20399 | CB | VAL | F | 295 | 79.518 | 14.258 | 4.400 | 1.00 | 84.54 | F |
| ATOM | 20400 | CG1 | VAL | F | 295 | 79.394 | 12.848 | 3.835 | 1.00 | 84.54 | F |
| ATOM | 20401 | CG2 | VAL | F | 295 | 78.931 | 15.284 | 3.442 | 1.00 | 84.54 | F |
| ATOM | 20402 | C | VAL | F | 295 | 79.497 | 13.461 | 6.766 | 1.00 | 62.88 | F |
| ATOM | 20403 | O | VAL | F | 295 | 79.293 | 12.249 | 6.793 | 1.00 | 62.88 | F |
| ATOM | 20404 | N | LYS | F | 296 | 80.323 | 14.084 | 7.602 | 1.00 | 66.66 | F |
| ATOM | 20405 | CA | LYS | F | 296 | 81.081 | 13.376 | 8.638 | 1.00 | 66.66 | F |
| ATOM | 20406 | CB | LYS | F | 296 | 81.939 | 14.371 | 9.430 | 1.00 | 109.87 | F |
| ATOM | 20407 | CG | LYS | F | 296 | 82.954 | 15.143 | 8.604 | 1.00 | 109.87 | F |
| ATOM | 20408 | CD | LYS | F | 296 | 84.128 | 14.270 | 8.201 | 1.00 | 109.87 | F |
| ATOM | 20409 | CE | LYS | F | 296 | 84.972 | 13.889 | 9.406 | 1.00 | 109.87 | F |
| ATOM | 20410 | NZ | LYS | F | 296 | 86.131 | 13.027 | 9.037 | 1.00 | 109.87 | F |
| ATOM | 20411 | C | LYS | F | 296 | 80.151 | 12.645 | 9.607 | 1.00 | 66.66 | F |
| ATOM | 20412 | O | LYS | F | 296 | 80.503 | 11.595 | 10.145 | 1.00 | 66.66 | F |
| ATOM | 20413 | N | ASN | F | 297 | 78.963 | 13.209 | 9.821 | 1.00 | 69.91 | F |
| ATOM | 20414 | CA | ASN | F | 297 | 77.990 | 12.636 | 10.744 | 1.00 | 69.91 | F |
| ATOM | 20415 | CB | ASN | F | 297 | 76.771 | 13.551 | 10.876 | 1.00 | 69.44 | F |
| ATOM | 20416 | CG | ASN | F | 297 | 75.849 | 13.136 | 12.015 | 1.00 | 69.44 | F |
| ATOM | 20417 | OD1 | ASN | F | 297 | 75.298 | 12.035 | 12.016 | 1.00 | 69.44 | F |
| ATOM | 20418 | ND2 | ASN | F | 297 | 75.681 | 14.019 | 12.990 | 1.00 | 69.44 | F |
| ATOM | 20419 | C | ASN | F | 297 | 77.527 | 11.241 | 10.362 | 1.00 | 69.91 | F |
| ATOM | 20420 | O | ASN | F | 297 | 77.231 | 10.951 | 9.199 | 1.00 | 69.91 | F |
| ATOM | 20421 | N | THR | F | 298 | 77.460 | 10.383 | 11.371 | 1.00 | 60.38 | F |
| ATOM | 20422 | CA | THR | F | 298 | 77.047 | 9.001 | 11.198 | 1.00 | 60.38 | F |
| ATOM | 20423 | CB | THR | F | 298 | 77.163 | 8.248 | 12.543 | 1.00 | 73.01 | F |
| ATOM | 20424 | OG1 | THR | F | 298 | 76.355 | 7.072 | 12.504 | 1.00 | 73.01 | F |
| ATOM | 20425 | CG2 | THR | F | 298 | 76.715 | 9.132 | 13.697 | 1.00 | 73.01 | F |
| ATOM | 20426 | C | THR | F | 298 | 75.630 | 8.847 | 10.633 | 1.00 | 60.38 | F |
| ATOM | 20427 | O | THR | F | 298 | 75.324 | 7.852 | 9.982 | 1.00 | 60.38 | F |
| ATOM | 20428 | N | LEU | F | 299 | 74.773 | 9.836 | 10.861 | 1.00 | 81.19 | F |
| ATOM | 20429 | CA | LEU | F | 299 | 73.397 | 9.772 | 10.367 | 1.00 | 81.19 | F |
| ATOM | 20430 | CB | LEU | F | 299 | 72.443 | 10.312 | 11.429 | 1.00 | 70.46 | F |
| ATOM | 20431 | CG | LEU | F | 299 | 72.380 | 9.469 | 12.702 | 1.00 | 70.46 | F |
| ATOM | 20432 | CD1 | LEU | F | 299 | 71.523 | 10.173 | 13.739 | 1.00 | 70.46 | F |
| ATOM | 20433 | CD2 | LEU | F | 299 | 71.816 | 8.103 | 12.376 | 1.00 | 70.46 | F |
| ATOM | 20434 | C | LEU | F | 299 | 73.158 | 10.516 | 9.052 | 1.00 | 81.19 | F |
| ATOM | 20435 | O | LEU | F | 299 | 72.016 | 10.635 | 8.594 | 1.00 | 81.19 | F |
| ATOM | 20436 | N | TRP | F | 300 | 74.235 | 11.006 | 8.446 | 1.00 | 68.46 | F |
| ATOM | 20437 | CA | TRP | F | 300 | 74.141 | 11.747 | 7.196 | 1.00 | 68.46 | F |
| ATOM | 20438 | CB | TRP | F | 300 | 75.531 | 11.977 | 6.608 | 1.00 | 77.70 | F |
| ATOM | 20439 | CG | TRP | F | 300 | 75.496 | 12.763 | 5.348 | 1.00 | 77.70 | F |
| ATOM | 20440 | CD2 | TRP | F | 300 | 74.991 | 14.091 | 5.181 | 1.00 | 77.70 | F |
| ATOM | 20441 | CE2 | TRP | F | 300 | 75.164 | 14.435 | 3.822 | 1.00 | 77.70 | F |
| ATOM | 20442 | CE3 | TRP | F | 300 | 74.411 | 15.027 | 6.049 | 1.00 | 77.70 | F |
| ATOM | 20443 | CD1 | TRP | F | 300 | 75.940 | 12.363 | 4.121 | 1.00 | 77.70 | F |
| ATOM | 20444 | NE1 | TRP | F | 300 | 75.745 | 13.362 | 3.199 | 1.00 | 77.70 | F |
| ATOM | 20445 | CZ2 | TRP | F | 300 | 74.776 | 15.678 | 3.306 | 1.00 | 77.70 | F |
| ATOM | 20446 | CZ3 | TRP | F | 300 | 74.025 | 16.264 | 5.537 | 1.00 | 77.70 | F |
| ATOM | 20447 | CH2 | TRP | F | 300 | 74.210 | 16.577 | 4.177 | 1.00 | 77.70 | F |
| ATOM | 20448 | C | TRP | F | 300 | 73.273 | 11.035 | 6.175 | 1.00 | 68.46 | F |
| ATOM | 20449 | O | TRP | F | 300 | 72.467 | 11.670 | 5.499 | 1.00 | 68.46 | F |
| ATOM | 20450 | N | LYS | F | 301 | 73.436 | 9.719 | 6.066 | 1.00 | 70.36 | F |
| ATOM | 20451 | CA | LYS | F | 301 | 72.659 | 8.948 | 5.107 | 1.00 | 70.36 | F |
| ATOM | 20452 | CB | LYS | F | 301 | 73.021 | 7.461 | 5.175 | 1.00 | 85.96 | F |
| ATOM | 20453 | CG | LYS | F | 301 | 74.493 | 7.170 | 4.875 | 1.00 | 85.96 | F |
| ATOM | 20454 | CD | LYS | F | 301 | 74.974 | 7.869 | 3.598 | 1.00 | 85.96 | F |
| ATOM | 20455 | CE | LYS | F | 301 | 76.497 | 7.925 | 3.532 | 1.00 | 85.96 | F |
| ATOM | 20456 | NZ | LYS | F | 301 | 76.982 | 8.940 | 2.552 | 1.00 | 85.96 | F |
| ATOM | 20457 | C | LYS | F | 301 | 71.165 | 9.136 | 5.327 | 1.00 | 70.36 | F |
| ATOM | 20458 | O | LYS | F | 301 | 70.392 | 9.185 | 4.366 | 1.00 | 70.36 | F |
| ATOM | 20459 | N | ASP | F | 302 | 70.750 | 9.258 | 6.583 | 1.00 | 68.24 | F |
| ATOM | 20460 | CA | ASP | F | 302 | 69.335 | 9.459 | 6.869 | 1.00 | 68.24 | F |
| ATOM | 20461 | CB | ASP | F | 302 | 69.043 | 9.185 | 8.340 | 1.00 | 122.37 | F |
| ATOM | 20462 | CG | ASP | F | 302 | 69.119 | 7.713 | 8.675 | 1.00 | 122.37 | F |
| ATOM | 20463 | OD1 | ASP | F | 302 | 68.479 | 6.914 | 7.959 | 1.00 | 122.37 | F |
| ATOM | 20464 | OD2 | ASP | F | 302 | 69.811 | 7.354 | 9.651 | 1.00 | 122.37 | F |
| ATOM | 20465 | C | ASP | F | 302 | 68.907 | 10.876 | 6.496 | 1.00 | 68.24 | F |
| ATOM | 20466 | O | ASP | F | 302 | 67.788 | 11.096 | 6.022 | 1.00 | 68.24 | F |
| ATOM | 20467 | N | VAL | F | 303 | 69.799 | 11.836 | 6.711 | 1.00 | 67.20 | F |
| ATOM | 20468 | CA | VAL | F | 303 | 69.500 | 13.216 | 6.360 | 1.00 | 67.20 | F |
| ATOM | 20469 | CB | VAL | F | 303 | 70.676 | 14.148 | 6.711 | 1.00 | 67.00 | F |
| ATOM | 20470 | CG1 | VAL | F | 303 | 70.402 | 15.556 | 6.202 | 1.00 | 56.31 | F |
| ATOM | 20471 | CG2 | VAL | F | 303 | 70.881 | 14.165 | 8.218 | 1.00 | 56.31 | F |
| ATOM | 20472 | C | VAL | F | 303 | 69.232 | 13.279 | 4.859 | 1.00 | 67.20 | F |
| ATOM | 20473 | O | VAL | F | 303 | 68.198 | 13.797 | 4.433 | 1.00 | 67.20 | F |
| ATOM | 20474 | N | LYS | F | 304 | 70.166 | 12.746 | 4.067 | 1.00 | 68.52 | F |
| ATOM | 20475 | CA | LYS | F | 304 | 70.036 | 12.727 | 2.611 | 1.00 | 68.52 | F |
| ATOM | 20476 | CB | LYS | F | 304 | 71.134 | 11.873 | 1.977 | 1.00 | 113.59 | F |
| ATOM | 20477 | CG | LYS | F | 304 | 72.454 | 12.566 | 1.742 | 1.00 | 95.51 | F |
| ATOM | 20478 | CD | LYS | F | 304 | 73.438 | 11.580 | 1.125 | 1.00 | 95.51 | F |
| ATOM | 20479 | CE | LYS | F | 304 | 74.759 | 12.250 | 0.762 | 1.00 | 95.51 | F |
| ATOM | 20480 | NZ | LYS | F | 304 | 75.794 | 11.274 | 0.301 | 1.00 | 95.51 | F |
| ATOM | 20481 | C | LYS | F | 304 | 68.689 | 12.145 | 2.212 | 1.00 | 68.52 | F |
| ATOM | 20482 | O | LYS | F | 304 | 67.979 | 12.693 | 1.367 | 1.00 | 68.52 | F |
| ATOM | 20483 | N | ALA | F | 305 | 68.334 | 11.031 | 2.835 | 1.00 | 48.30 | F |
| ATOM | 20484 | CA | ALA | F | 305 | 67.074 | 10.366 | 2.519 | 1.00 | 48.30 | F |
| ATOM | 20485 | CB | ALA | F | 305 | 67.061 | 8.982 | 3.100 | 1.00 | 1.84 | F |
| ATOM | 20486 | C | ALA | F | 305 | 65.821 | 11.099 | 2.956 | 1.00 | 48.30 | F |
| ATOM | 20487 | O | ALA | F | 305 | 64.908 | 11.285 | 2.166 | 1.00 | 48.30 | F |
| ATOM | 20488 | N | TYR | F | 306 | 65.764 | 11.505 | 4.218 | 1.00 | 56.26 | F |
| ATOM | 20489 | CA | TYR | F | 306 | 64.588 | 12.206 | 4.723 | 1.00 | 56.26 | F |
| ATOM | 20490 | CB | TYR | F | 306 | 64.621 | 12.248 | 6.245 | 1.00 | 78.60 | F |
| ATOM | 20491 | CG | TYR | F | 306 | 64.486 | 10.893 | 6.879 | 1.00 | 73.29 | F |
| ATOM | 20492 | CD1 | TYR | F | 306 | 65.063 | 10.622 | 8.119 | 1.00 | 73.29 | F |
| ATOM | 20493 | CE1 | TYR | F | 306 | 64.929 | 9.383 | 8.717 | 1.00 | 73.29 | F |
| ATOM | 20494 | CD2 | TYR | F | 306 | 63.768 | 9.885 | 6.250 | 1.00 | 73.29 | F |
| ATOM | 20495 | CE2 | TYR | F | 306 | 63.626 | 8.642 | 6.837 | 1.00 | 73.29 | F |
| ATOM | 20496 | CZ | TYR | F | 306 | 64.208 | 8.397 | 8.071 | 1.00 | 73.29 | F |
| ATOM | 20497 | OH | TYR | F | 306 | 64.054 | 7.165 | 8.663 | 1.00 | 73.29 | F |
| ATOM | 20498 | C | TYR | F | 306 | 64.438 | 13.619 | 4.183 | 1.00 | 56.26 | F |
| ATOM | 20499 | O | TYR | F | 306 | 63.380 | 13.976 | 3.690 | 1.00 | 56.26 | F |
| ATOM | 20500 | N | LEU | F | 307 | 65.493 | 14.418 | 4.277 | 1.00 | 62.69 | F |
| ATOM | 20501 | CA | LEU | F | 307 | 65.439 | 15.794 | 3.809 | 1.00 | 62.69 | F |
| ATOM | 20502 | CB | LEU | F | 307 | 66.509 | 16.631 | 4.511 | 1.00 | 72.81 | F |
| ATOM | 20503 | CG | LEU | F | 307 | 66.684 | 18.054 | 3.986 | 1.00 | 40.94 | F |
| ATOM | 20504 | CD1 | LEU | F | 307 | 65.379 | 18.836 | 4.133 | 1.00 | 40.94 | F |
| ATOM | 20505 | CD2 | LEU | F | 307 | 67.811 | 18.738 | 4.740 | 1.00 | 40.94 | F |
| ATOM | 20506 | C | LEU | F | 307 | 65.622 | 15.879 | 2.301 | 1.00 | 62.69 | F |
| ATOM | 20507 | O | LEU | F | 307 | 64.807 | 16.483 | 1.597 | 1.00 | 62.69 | F |
| ATOM | 20508 | N | GLY | F | 308 | 66.692 | 15.270 | 1.806 | 1.00 | 57.27 | F |
| ATOM | 20509 | CA | GLY | F | 308 | 66.946 | 15.303 | 0.381 | 1.00 | 57.27 | F |
| ATOM | 20510 | C | GLY | F | 308 | 65.799 | 14.711 | −0.422 | 1.00 | 57.27 | F |
| ATOM | 20511 | O | GLY | F | 308 | 65.342 | 15.310 | −1.393 | 1.00 | 57.27 | F |
| ATOM | 20512 | N | GLY | F | 309 | 65.327 | 13.538 | −0.014 | 1.00 | 56.54 | F |
| ATOM | 20513 | CA | GLY | F | 309 | 64.245 | 12.889 | −0.730 | 1.00 | 56.54 | F |
| ATOM | 20514 | C | GLY | F | 309 | 62.988 | 13.727 | −0.806 | 1.00 | 56.54 | F |
| ATOM | 20515 | O | GLY | F | 309 | 62.409 | 13.909 | −1.877 | 1.00 | 56.54 | F |
| ATOM | 20516 | N | PHE | F | 310 | 62.565 | 14.244 | 0.339 | 1.00 | 54.21 | F |
| ATOM | 20517 | CA | PHE | F | 310 | 61.364 | 15.058 | 0.416 | 1.00 | 54.21 | F |
| ATOM | 20518 | CB | PHE | F | 310 | 61.172 | 15.530 | 1.857 | 1.00 | 57.82 | F |
| ATOM | 20519 | CG | PHE | F | 310 | 59.844 | 16.171 | 2.119 | 1.00 | 53.65 | F |
| ATOM | 20520 | CD1 | PHE | F | 310 | 58.673 | 15.445 | 1.966 | 1.00 | 53.65 | F |
| ATOM | 20521 | CD2 | PHE | F | 310 | 59.766 | 17.502 | 2.529 | 1.00 | 53.65 | F |
| ATOM | 20522 | CE1 | PHE | F | 310 | 57.433 | 16.031 | 2.218 | 1.00 | 53.65 | F |
| ATOM | 20523 | CE2 | PHE | F | 310 | 58.532 | 18.098 | 2.785 | 1.00 | 53.65 | F |
| ATOM | 20524 | CZ | PHE | F | 310 | 57.361 | 17.357 | 2.627 | 1.00 | 53.65 | F |
| ATOM | 20525 | C | PHE | F | 310 | 61.493 | 16.263 | −0.513 | 1.00 | 54.21 | F |
| ATOM | 20526 | O | PHE | F | 310 | 60.559 | 16.602 | −1.252 | 1.00 | 54.21 | F |
| ATOM | 20527 | N | GLU | F | 311 | 62.661 | 16.899 | −0.466 | 1.00 | 55.62 | F |
| ATOM | 20528 | CA | GLU | F | 311 | 62.928 | 18.070 | −1.279 | 1.00 | 55.62 | F |
| ATOM | 20529 | CB | GLU | F | 311 | 64.205 | 18.756 | −0.806 | 1.00 | 79.14 | F |
| ATOM | 20530 | CG | GLU | F | 311 | 64.261 | 20.211 | −1.192 | 1.00 | 63.37 | F |
| ATOM | 20531 | CD | GLU | F | 311 | 65.604 | 20.848 | −0.909 | 1.00 | 63.37 | F |
| ATOM | 20532 | OE1 | GLU | F | 311 | 66.205 | 20.524 | 0.125 | 1.00 | 63.37 | F |
| ATOM | 20533 | OE2 | GLU | F | 311 | 66.056 | 21.691 | −1.710 | 1.00 | 63.37 | F |
| ATOM | 20534 | C | GLU | F | 311 | 63.064 | 17.712 | −2.756 | 1.00 | 55.62 | F |
| ATOM | 20535 | O | GLU | F | 311 | 62.693 | 18.497 | −3.624 | 1.00 | 55.62 | F |
| ATOM | 20536 | N | TYR | F | 312 | 63.593 | 16.526 | −3.041 | 1.00 | 67.90 | F |
| ATOM | 20537 | CA | TYR | F | 312 | 63.775 | 16.105 | −4.423 | 1.00 | 67.90 | F |
| ATOM | 20538 | CB | TYR | F | 312 | 64.671 | 14.878 | −4.508 | 1.00 | 60.43 | F |
| ATOM | 20539 | CG | TYR | F | 312 | 64.950 | 14.462 | −5.930 | 1.00 | 55.13 | F |
| ATOM | 20540 | CD1 | TYR | F | 312 | 65.818 | 15.204 | −6.731 | 1.00 | 55.13 | F |
| ATOM | 20541 | CE1 | TYR | F | 312 | 66.052 | 14.855 | −8.052 | 1.00 | 55.13 | F |
| ATOM | 20542 | CD2 | TYR | F | 312 | 64.314 | 13.350 | −6.489 | 1.00 | 55.13 | F |
| ATOM | 20543 | CE2 | TYR | F | 312 | 64.537 | 12.987 | −7.816 | 1.00 | 55.13 | F |
| ATOM | 20544 | CZ | TYR | F | 312 | 65.409 | 13.746 | −8.594 | 1.00 | 55.13 | F |
| ATOM | 20545 | OH | TYR | F | 312 | 65.642 | 13.408 | −9.916 | 1.00 | 55.13 | F |
| ATOM | 20546 | C | TYR | F | 312 | 62.480 | 15.791 | −5.142 | 1.00 | 67.90 | F |
| ATOM | 20547 | O | TYR | F | 312 | 62.347 | 16.072 | −6.331 | 1.00 | 67.90 | F |
| ATOM | 20548 | N | MET | F | 313 | 61.523 | 15.202 | −4.439 | 1.00 | 54.84 | F |
| ATOM | 20549 | CA | MET | F | 313 | 60.274 | 14.866 | −5.093 | 1.00 | 54.84 | F |
| ATOM | 20550 | CB | MET | F | 313 | 60.483 | 13.656 | −6.001 | 1.00 | 81.95 | F |
| ATOM | 20551 | CG | MET | F | 313 | 59.248 | 13.264 | −6.774 | 1.00 | 81.80 | F |
| ATOM | 20552 | SD | MET | F | 313 | 59.501 | 11.779 | −7.722 | 1.00 | 81.80 | F |
| ATOM | 20553 | CE | MET | F | 313 | 60.905 | 12.266 | −8.725 | 1.00 | 81.80 | F |
| ATOM | 20554 | C | MET | F | 313 | 59.129 | 14.579 | −4.137 | 1.00 | 54.84 | F |
| ATOM | 20555 | O | MET | F | 313 | 57.970 | 14.873 | −4.437 | 1.00 | 54.84 | F |
| ATOM | 20556 | N | GLY | F | 314 | 59.453 | 13.997 | −2.990 | 1.00 | 50.56 | F |
| ATOM | 20557 | CA | GLY | F | 314 | 58.429 | 13.658 | −2.025 | 1.00 | 50.56 | F |
| ATOM | 20558 | C | GLY | F | 314 | 57.408 | 14.750 | −1.776 | 1.00 | 50.56 | F |
| ATOM | 20559 | O | GLY | F | 314 | 56.199 | 14.530 | −1.883 | 1.00 | 50.56 | F |
| ATOM | 20560 | N | CYS | F | 315 | 57.893 | 15.939 | −1.447 | 1.00 | 68.24 | F |
| ATOM | 20561 | CA | CYS | F | 315 | 57.005 | 17.047 | −1.163 | 1.00 | 68.24 | F |
| ATOM | 20562 | CB | CYS | F | 315 | 57.822 | 18.271 | −0.774 | 1.00 | 77.00 | F |
| ATOM | 20563 | SG | CYS | F | 315 | 56.825 | 19.549 | −0.022 | 1.00 | 82.37 | F |
| ATOM | 20564 | C | CYS | F | 315 | 56.073 | 17.389 | −2.326 | 1.00 | 68.24 | F |
| ATOM | 20565 | O | CYS | F | 315 | 54.855 | 17.466 | −2.150 | 1.00 | 68.24 | F |
| ATOM | 20566 | N | SER | F | 316 | 56.639 | 17.585 | −3.514 | 1.00 | 53.61 | F |
| ATOM | 20567 | CA | SER | F | 316 | 55.836 | 17.937 | −4.680 | 1.00 | 53.61 | F |
| ATOM | 20568 | CB | SER | F | 316 | 56.716 | 18.071 | −5.920 | 1.00 | 54.17 | F |
| ATOM | 20569 | OG | SER | F | 316 | 57.224 | 16.813 | −6.304 | 1.00 | 59.67 | F |
| ATOM | 20570 | C | SER | F | 316 | 54.727 | 16.933 | −4.955 | 1.00 | 53.61 | F |
| ATOM | 20571 | O | SER | F | 316 | 53.710 | 17.276 | −5.552 | 1.00 | 53.61 | F |
| ATOM | 20572 | N | LEU | F | 317 | 54.921 | 15.690 | −4.539 | 1.00 | 60.08 | F |
| ATOM | 20573 | CA | LEU | F | 317 | 53.896 | 14.679 | −4.746 | 1.00 | 60.08 | F |
| ATOM | 20574 | CB | LEU | F | 317 | 54.516 | 13.286 | −4.745 | 1.00 | 58.95 | F |
| ATOM | 20575 | CG | LEU | F | 317 | 55.366 | 12.931 | −5.960 | 1.00 | 51.46 | F |
| ATOM | 20576 | CD1 | LEU | F | 317 | 56.063 | 11.592 | −5.757 | 1.00 | 51.46 | F |
| ATOM | 20577 | CD2 | LEU | F | 317 | 54.476 | 12.874 | −7.167 | 1.00 | 51.46 | F |
| ATOM | 20578 | C | LEU | F | 317 | 52.858 | 14.774 | −3.634 | 1.00 | 60.08 | F |
| ATOM | 20579 | O | LEU | F | 317 | 51.650 | 14.690 | −3.876 | 1.00 | 60.08 | F |
| ATOM | 20580 | N | ALA | F | 318 | 53.339 | 14.958 | −2.411 | 1.00 | 61.98 | F |
| ATOM | 20581 | CA | ALA | F | 318 | 52.466 | 15.047 | −1.257 | 1.00 | 61.98 | F |
| ATOM | 20582 | CB | ALA | F | 318 | 53.307 | 15.125 | 0.008 | 1.00 | 70.04 | F |
| ATOM | 20583 | C | ALA | F | 318 | 51.523 | 16.245 | −1.334 | 1.00 | 61.98 | F |
| ATOM | 20584 | O | ALA | F | 318 | 50.319 | 16.109 | −1.111 | 1.00 | 61.98 | F |
| ATOM | 20585 | N | ILE | F | 319 | 52.066 | 17.416 | −1.654 | 1.00 | 70.43 | F |
| ATOM | 20586 | CA | ILE | F | 319 | 51.252 | 18.619 | −1.711 | 1.00 | 70.43 | F |
| ATOM | 20587 | CB | ILE | F | 319 | 52.027 | 19.805 | −2.321 | 1.00 | 76.92 | F |
| ATOM | 20588 | CG2 | ILE | F | 319 | 52.222 | 19.592 | −3.806 | 1.00 | 51.48 | F |
| ATOM | 20589 | CG1 | ILE | F | 319 | 51.246 | 21.105 | −2.085 | 1.00 | 51.48 | F |
| ATOM | 20590 | CD1 | ILE | F | 319 | 51.993 | 22.380 | −2.490 | 1.00 | 51.48 | F |
| ATOM | 20591 | C | ILE | F | 319 | 49.961 | 18.405 | −2.491 | 1.00 | 70.43 | F |
| ATOM | 20592 | O | ILE | F | 319 | 48.948 | 19.025 | −2.188 | 1.00 | 70.43 | F |
| ATOM | 20593 | N | LEU | F | 320 | 49.988 | 17.521 | −3.482 | 1.00 | 90.03 | F |
| ATOM | 20594 | CA | LEU | F | 320 | 48.794 | 17.258 | −4.276 | 1.00 | 90.03 | F |
| ATOM | 20595 | CB | LEU | F | 320 | 49.166 | 16.507 | −5.556 | 1.00 | 92.15 | F |
| ATOM | 20596 | CG | LEU | F | 320 | 50.050 | 17.298 | −6.533 | 1.00 | 69.99 | F |
| ATOM | 20597 | CD1 | LEU | F | 320 | 50.317 | 16.452 | −7.776 | 1.00 | 69.99 | F |
| ATOM | 20598 | CD2 | LEU | F | 320 | 49.376 | 18.619 | −6.917 | 1.00 | 69.99 | F |
| ATOM | 20599 | C | LEU | F | 320 | 47.728 | 16.490 | −3.491 | 1.00 | 90.03 | F |
| ATOM | 20600 | O | LEU | F | 320 | 46.532 | 16.664 | −3.732 | 1.00 | 90.03 | F |
| ATOM | 20601 | N | ASP | F | 321 | 48.154 | 15.642 | −2.557 | 1.00 | 76.18 | F |
| ATOM | 20602 | CA | ASP | F | 321 | 47.202 | 14.896 | −1.737 | 1.00 | 76.18 | F |
| ATOM | 20603 | CB | ASP | F | 321 | 47.905 | 13.777 | −0.962 | 1.00 | 162.50 | F |
| ATOM | 20604 | CG | ASP | F | 321 | 48.356 | 12.639 | −1.858 | 1.00 | 141.50 | F |
| ATOM | 20605 | OD1 | ASP | F | 321 | 47.557 | 12.223 | −2.725 | 1.00 | 141.50 | F |
| ATOM | 20606 | OD2 | ASP | F | 321 | 49.498 | 12.152 | −1.686 | 1.00 | 141.50 | F |
| ATOM | 20607 | C | ASP | F | 321 | 46.521 | 15.858 | −0.754 | 1.00 | 76.18 | F |
| ATOM | 20608 | O | ASP | F | 321 | 45.326 | 15.737 | −0.480 | 1.00 | 76.18 | F |
| ATOM | 20609 | N | TYR | F | 322 | 47.282 | 16.814 | −0.225 | 1.00 | 94.02 | F |
| ATOM | 20610 | CA | TYR | F | 322 | 46.725 | 17.787 | 0.712 | 1.00 | 94.02 | F |
| ATOM | 20611 | CB | TYR | F | 322 | 47.797 | 18.759 | 1.219 | 1.00 | 87.06 | F |
| ATOM | 20612 | CG | TYR | F | 322 | 48.749 | 18.195 | 2.256 | 1.00 | 77.77 | F |
| ATOM | 20613 | CD1 | TYR | F | 322 | 49.705 | 17.242 | 1.911 | 1.00 | 77.77 | F |
| ATOM | 20614 | CE1 | TYR | F | 322 | 50.613 | 16.756 | 2.854 | 1.00 | 77.77 | F |
| ATOM | 20615 | CD2 | TYR | F | 322 | 48.718 | 18.645 | 3.575 | 1.00 | 77.77 | F |
| ATOM | 20616 | CE2 | TYR | F | 322 | 49.619 | 18.164 | 4.523 | 1.00 | 77.77 | F |
| ATOM | 20617 | CZ | TYR | F | 322 | 50.564 | 17.223 | 4.154 | 1.00 | 77.77 | F |
| ATOM | 20618 | OH | TYR | F | 322 | 51.476 | 16.758 | 5.071 | 1.00 | 77.77 | F |
| ATOM | 20619 | C | TYR | F | 322 | 45.650 | 18.581 | −0.002 | 1.00 | 94.02 | F |
| ATOM | 20620 | O | TYR | F | 322 | 44.546 | 18.766 | 0.511 | 1.00 | 94.02 | F |
| ATOM | 20621 | N | GLN | F | 323 | 45.987 | 19.051 | −1.195 | 1.00 | 107.93 | F |
| ATOM | 20622 | CA | GLN | F | 323 | 45.058 | 19.827 | −1.990 | 1.00 | 107.93 | F |
| ATOM | 20623 | CB | GLN | F | 323 | 45.664 | 20.128 | −3.353 | 1.00 | 104.55 | F |
| ATOM | 20624 | CG | GLN | F | 323 | 44.749 | 20.934 | −4.230 | 1.00 | 78.94 | F |
| ATOM | 20625 | CD | GLN | F | 323 | 45.395 | 21.317 | −5.531 | 1.00 | 78.94 | F |
| ATOM | 20626 | OE1 | GLN | F | 323 | 45.927 | 20.467 | −6.243 | 1.00 | 78.94 | F |
| ATOM | 20627 | NE2 | GLN | F | 323 | 45.351 | 22.605 | −5.858 | 1.00 | 78.94 | F |
| ATOM | 20628 | C | GLN | F | 323 | 43.726 | 19.104 | −2.160 | 1.00 | 107.93 | F |
| ATOM | 20629 | O | GLN | F | 323 | 42.672 | 19.708 | −1.986 | 1.00 | 107.93 | F |
| ATOM | 20630 | N | LYS | F | 324 | 43.771 | 17.816 | −2.498 | 1.00 | 83.47 | F |
| ATOM | 20631 | CA | LYS | F | 324 | 42.551 | 17.029 | −2.672 | 1.00 | 83.47 | F |
| ATOM | 20632 | CB | LYS | F | 324 | 42.875 | 15.576 | −3.007 | 1.00 | 85.53 | F |
| ATOM | 20633 | CG | LYS | F | 324 | 43.281 | 15.323 | −4.428 | 1.00 | 74.96 | F |
| ATOM | 20634 | CD | LYS | F | 324 | 43.296 | 13.825 | −4.697 | 1.00 | 74.96 | F |
| ATOM | 20635 | CE | LYS | F | 324 | 43.676 | 13.514 | −6.145 | 1.00 | 74.96 | F |
| ATOM | 20636 | NZ | LYS | F | 324 | 43.539 | 12.066 | −6.484 | 1.00 | 74.96 | F |
| ATOM | 20637 | C | LYS | F | 324 | 41.679 | 17.032 | −1.426 | 1.00 | 83.47 | F |
| ATOM | 20638 | O | LYS | F | 324 | 40.485 | 17.305 | −1.497 | 1.00 | 83.47 | F |
| ATOM | 20639 | N | MET | F | 325 | 42.274 | 16.708 | −0.286 | 1.00 | 86.99 | F |
| ATOM | 20640 | CA | MET | F | 325 | 41.528 | 16.661 | 0.958 | 1.00 | 86.99 | F |
| ATOM | 20641 | CB | MET | F | 325 | 42.315 | 15.887 | 2.005 | 1.00 | 103.64 | F |
| ATOM | 20642 | CG | MET | F | 325 | 42.513 | 14.441 | 1.639 | 1.00 | 92.20 | F |
| ATOM | 20643 | SD | MET | F | 325 | 43.448 | 13.539 | 2.881 | 1.00 | 92.20 | F |
| ATOM | 20644 | CE | MET | F | 325 | 45.075 | 13.620 | 2.194 | 1.00 | 92.20 | F |
| ATOM | 20645 | C | MET | F | 325 | 41.162 | 18.028 | 1.510 | 1.00 | 86.99 | F |
| ATOM | 20646 | O | MET | F | 325 | 40.324 | 18.130 | 2.401 | 1.00 | 86.99 | F |
| ATOM | 20647 | N | ALA | F | 326 | 41.777 | 19.079 | 0.989 | 1.00 | 92.07 | F |
| ATOM | 20648 | CA | ALA | F | 326 | 41.470 | 20.411 | 1.482 | 1.00 | 92.07 | F |
| ATOM | 20649 | CB | ALA | F | 326 | 42.741 | 21.267 | 1.504 | 1.00 | 50.53 | F |
| ATOM | 20650 | C | ALA | F | 326 | 40.386 | 21.098 | 0.651 | 1.00 | 92.07 | F |
| ATOM | 20651 | O | ALA | F | 326 | 39.670 | 21.967 | 1.154 | 1.00 | 92.07 | F |
| ATOM | 20652 | N | LYS | F | 327 | 40.259 | 20.687 | −0.608 | 1.00 | 143.74 | F |
| ATOM | 20653 | CA | LYS | F | 327 | 39.301 | 21.278 | −1.538 | 1.00 | 143.74 | F |
| ATOM | 20654 | CB | LYS | F | 327 | 38.732 | 20.220 | −2.484 | 1.00 | 173.39 | F |
| ATOM | 20655 | CG | LYS | F | 327 | 37.983 | 20.844 | −3.649 | 1.00 | 91.68 | F |
| ATOM | 20656 | CD | LYS | F | 327 | 37.542 | 19.819 | −4.672 | 1.00 | 91.68 | F |
| ATOM | 20657 | CE | LYS | F | 327 | 36.893 | 20.491 | −5.880 | 1.00 | 91.68 | F |
| ATOM | 20658 | NZ | LYS | F | 327 | 36.409 | 19.495 | −6.882 | 1.00 | 91.68 | F |
| ATOM | 20659 | C | LYS | F | 327 | 38.149 | 22.053 | −0.916 | 1.00 | 143.74 | F |
| ATOM | 20660 | O | LYS | F | 327 | 38.072 | 23.271 | −1.057 | 1.00 | 143.74 | F |
| ATOM | 20661 | N | GLY | F | 328 | 37.255 | 21.364 | −0.222 | 1.00 | 90.23 | F |
| ATOM | 20662 | CA | GLY | F | 328 | 36.129 | 22.070 | 0.359 | 1.00 | 90.23 | F |
| ATOM | 20663 | C | GLY | F | 328 | 36.293 | 22.692 | 1.735 | 1.00 | 90.23 | F |
| ATOM | 20664 | O | GLY | F | 328 | 35.599 | 23.655 | 2.051 | 1.00 | 90.23 | F |
| ATOM | 20665 | N | LYS | F | 329 | 37.213 | 22.181 | 2.546 | 1.00 | 95.68 | F |
| ATOM | 20666 | CA | LYS | F | 329 | 37.368 | 22.691 | 3.902 | 1.00 | 95.68 | F |
| ATOM | 20667 | CB | LYS | F | 329 | 37.741 | 21.535 | 4.833 | 1.00 | 175.53 | F |
| ATOM | 20668 | CG | LYS | F | 329 | 36.653 | 20.484 | 4.958 | 1.00 | 175.53 | F |
| ATOM | 20669 | CD | LYS | F | 329 | 36.876 | 19.633 | 6.191 | 1.00 | 175.53 | F |
| ATOM | 20670 | CE | LYS | F | 329 | 35.766 | 18.620 | 6.397 | 1.00 | 65.29 | F |
| ATOM | 20671 | NZ | LYS | F | 329 | 35.991 | 17.818 | 7.645 | 1.00 | 65.29 | F |
| ATOM | 20672 | C | LYS | F | 329 | 38.264 | 23.893 | 4.218 | 1.00 | 95.68 | F |
| ATOM | 20673 | O | LYS | F | 329 | 38.605 | 24.102 | 5.382 | 1.00 | 95.68 | F |
| ATOM | 20674 | N | ILE | F | 330 | 38.650 | 24.690 | 3.225 | 1.00 | 109.00 | F |
| ATOM | 20675 | CA | ILE | F | 330 | 39.477 | 25.868 | 3.519 | 1.00 | 109.00 | F |
| ATOM | 20676 | CB | ILE | F | 330 | 40.996 | 25.611 | 3.271 | 1.00 | 107.63 | F |
| ATOM | 20677 | CG2 | ILE | F | 330 | 41.461 | 24.394 | 4.061 | 1.00 | 108.55 | F |
| ATOM | 20678 | CG1 | ILE | F | 330 | 41.269 | 25.429 | 1.780 | 1.00 | 108.55 | F |
| ATOM | 20679 | CD1 | ILE | F | 330 | 42.729 | 25.549 | 1.425 | 1.00 | 108.55 | F |
| ATOM | 20680 | C | ILE | F | 330 | 39.036 | 27.062 | 2.675 | 1.00 | 109.00 | F |
| ATOM | 20681 | O | ILE | F | 330 | 38.227 | 26.912 | 1.760 | 1.00 | 109.00 | F |
| ATOM | 20682 | N | PRO | F | 331 | 39.559 | 28.267 | 2.974 | 1.00 | 113.13 | F |
| ATOM | 20683 | CD | PRO | F | 331 | 40.553 | 28.596 | 4.013 | 1.00 | 63.98 | F |
| ATOM | 20684 | CA | PRO | F | 331 | 39.190 | 29.467 | 2.215 | 1.00 | 113.13 | F |
| ATOM | 20685 | CB | PRO | F | 331 | 40.243 | 30.482 | 2.644 | 1.00 | 200.84 | F |
| ATOM | 20686 | CG | PRO | F | 331 | 40.487 | 30.115 | 4.051 | 1.00 | 63.98 | F |
| ATOM | 20687 | C | PRO | F | 331 | 39.217 | 29.224 | 0.712 | 1.00 | 113.13 | F |
| ATOM | 20688 | O | PRO | F | 331 | 40.144 | 28.606 | 0.198 | 1.00 | 113.13 | F |
| ATOM | 20689 | N | LYS | F | 332 | 38.193 | 29.705 | 0.015 | 1.00 | 94.59 | F |
| ATOM | 20690 | CA | LYS | F | 332 | 38.111 | 29.545 | −1.431 | 1.00 | 95.21 | F |
| ATOM | 20691 | CB | LYS | F | 332 | 36.714 | 29.921 | −1.922 | 1.00 | 145.40 | F |
| ATOM | 20692 | CG | LYS | F | 332 | 36.348 | 31.356 | −1.610 | 1.00 | 116.94 | F |
| ATOM | 20693 | CD | LYS | F | 332 | 35.220 | 31.851 | −2.488 | 1.00 | 116.94 | F |
| ATOM | 20694 | CE | LYS | F | 332 | 35.020 | 33.351 | −2.317 | 1.00 | 116.94 | F |
| ATOM | 20695 | NZ | LYS | F | 332 | 34.006 | 33.882 | −3.264 | 1.00 | 116.94 | F |
| ATOM | 20696 | C | LYS | F | 332 | 39.140 | 30.453 | −2.104 | 1.00 | 94.28 | F |
| ATOM | 20697 | O | LYS | F | 332 | 39.595 | 31.442 | −1.519 | 1.00 | 92.37 | F |
| ATOM | 20698 | N | ASP | F | 333 | 39.513 | 30.114 | −3.332 | 1.00 | 99.44 | F |
| ATOM | 20699 | CA | ASP | F | 333 | 40.473 | 30.922 | −4.072 | 1.00 | 98.90 | F |
| ATOM | 20700 | CB | ASP | F | 333 | 40.796 | 30.264 | −5.411 | 1.00 | 180.51 | F |
| ATOM | 20701 | CG | ASP | F | 333 | 41.964 | 29.314 | −5.319 | 1.00 | 148.10 | F |
| ATOM | 20702 | OD1 | ASP | F | 333 | 42.090 | 28.627 | −4.286 | 1.00 | 148.10 | F |
| ATOM | 20703 | OD2 | ASP | F | 333 | 42.753 | 29.249 | −6.285 | 1.00 | 148.10 | F |
| ATOM | 20704 | C | ASP | F | 333 | 39.880 | 32.298 | −4.312 | 1.00 | 102.75 | F |
| ATOM | 20705 | O | ASP | F | 333 | 38.683 | 32.432 | −4.564 | 1.00 | 102.40 | F |
| ATOM | 20706 | N | THR | F | 334 | 40.716 | 33.324 | −4.226 | 1.00 | 87.13 | F |
| ATOM | 20707 | CA | THR | F | 334 | 40.254 | 34.687 | −4.443 | 1.00 | 86.85 | F |
| ATOM | 20708 | CB | THR | F | 334 | 40.027 | 35.412 | −3.102 | 1.00 | 108.13 | F |
| ATOM | 20709 | OG1 | THR | F | 334 | 38.830 | 34.909 | −2.495 | 1.00 | 94.03 | F |
| ATOM | 20710 | CG2 | THR | F | 334 | 39.906 | 36.922 | −3.311 | 1.00 | 94.03 | F |
| ATOM | 20711 | C | THR | F | 334 | 41.252 | 35.463 | −5.282 | 1.00 | 93.29 | F |
| ATOM | 20712 | O | THR | F | 334 | 42.414 | 35.623 | −4.901 | 1.00 | 90.65 | F |
| ATOM | 20713 | N | THR | F | 335 | 40.784 | 35.947 | −6.427 | 1.00 | 69.99 | F |
| ATOM | 20714 | CA | THR | F | 335 | 41.633 | 36.696 | −7.338 | 1.00 | 69.78 | F |
| ATOM | 20715 | CB | THR | F | 335 | 41.022 | 36.749 | −8.727 | 1.00 | 70.88 | F |
| ATOM | 20716 | OG1 | THR | F | 335 | 40.561 | 35.439 | −9.093 | 1.00 | 60.24 | F |
| ATOM | 20717 | CG2 | THR | F | 335 | 42.067 | 37.222 | −9.728 | 1.00 | 60.24 | F |
| ATOM | 20718 | C | THR | F | 335 | 41.899 | 38.115 | −6.869 | 1.00 | 70.64 | F |
| ATOM | 20719 | O | THR | F | 335 | 40.985 | 38.830 | −6.476 | 1.00 | 71.99 | F |
| ATOM | 20720 | N | PRO | F | 336 | 43.165 | 38.547 | −6.928 | 1.00 | 84.30 | F |
| ATOM | 20721 | CD | PRO | F | 336 | 44.364 | 37.799 | −7.341 | 1.00 | 64.90 | F |
| ATOM | 20722 | CA | PRO | F | 336 | 43.536 | 39.889 | −6.495 | 1.00 | 83.88 | F |
| ATOM | 20723 | CB | PRO | F | 336 | 45.007 | 39.729 | −6.169 | 1.00 | 73.76 | F |
| ATOM | 20724 | CG | PRO | F | 336 | 45.455 | 38.866 | −7.287 | 1.00 | 64.90 | F |
| ATOM | 20725 | C | PRO | F | 336 | 43.319 | 40.972 | −7.531 | 1.00 | 83.92 | F |
| ATOM | 20726 | O | PRO | F | 336 | 42.973 | 40.703 | −8.680 | 1.00 | 84.55 | F |
| ATOM | 20727 | N | THR | F | 337 | 43.543 | 42.202 | −7.086 | 1.00 | 79.46 | F |
| ATOM | 20728 | CA | THR | F | 337 | 43.436 | 43.394 | −7.904 | 1.00 | 82.92 | F |
| ATOM | 20729 | CB | THR | F | 337 | 42.545 | 44.444 | −7.225 | 1.00 | 124.18 | F |
| ATOM | 20730 | OG1 | THR | F | 337 | 41.180 | 44.017 | −7.279 | 1.00 | 75.07 | F |
| ATOM | 20731 | CG2 | THR | F | 337 | 42.696 | 45.798 | −7.901 | 1.00 | 75.07 | F |
| ATOM | 20732 | C | THR | F | 337 | 44.861 | 43.910 | −7.942 | 1.00 | 90.49 | F |
| ATOM | 20733 | O | THR | F | 337 | 45.519 | 43.997 | −6.902 | 1.00 | 81.87 | F |
| ATOM | 20734 | N | VAL | F | 338 | 45.355 | 44.242 | −9.125 | 1.00 | 83.98 | F |
| ATOM | 20735 | CA | VAL | F | 338 | 46.722 | 44.729 | −9.215 | 1.00 | 90.84 | F |
| ATOM | 20736 | CB | VAL | F | 338 | 47.608 | 43.782 | −10.064 | 1.00 | 92.16 | F |
| ATOM | 20737 | CG1 | VAL | F | 338 | 49.076 | 44.187 | −9.938 | 1.00 | 91.78 | F |
| ATOM | 20738 | CG2 | VAL | F | 338 | 47.410 | 42.343 | −9.617 | 1.00 | 91.78 | F |
| ATOM | 20739 | C | VAL | F | 338 | 46.782 | 46.122 | −9.810 | 1.00 | 95.31 | F |
| ATOM | 20740 | O | VAL | F | 338 | 45.963 | 46.483 | −10.651 | 1.00 | 95.14 | F |
| ATOM | 20741 | N | LYS | F | 339 | 47.761 | 46.896 | −9.353 | 1.00 | 77.33 | F |
| ATOM | 20742 | CA | LYS | F | 339 | 47.980 | 48.260 | −9.814 | 1.00 | 75.21 | F |
| ATOM | 20743 | CB | LYS | F | 339 | 47.118 | 49.233 | −9.008 | 1.00 | 167.31 | F |
| ATOM | 20744 | CG | LYS | F | 339 | 45.623 | 49.037 | −9.212 | 1.00 | 135.62 | F |
| ATOM | 20745 | CD | LYS | F | 339 | 44.824 | 49.379 | −7.962 | 1.00 | 135.62 | F |
| ATOM | 20746 | CE | LYS | F | 339 | 43.354 | 49.018 | −8.141 | 1.00 | 135.62 | F |
| ATOM | 20747 | NZ | LYS | F | 339 | 42.582 | 49.136 | −6.872 | 1.00 | 135.62 | F |
| ATOM | 20748 | C | LYS | F | 339 | 49.446 | 48.553 | −9.580 | 1.00 | 84.20 | F |
| ATOM | 20749 | O | LYS | F | 339 | 49.977 | 48.266 | −8.509 | 1.00 | 76.86 | F |
| ATOM | 20750 | N | VAL | F | 340 | 50.112 | 49.097 | −10.585 | 1.00 | 92.91 | F |
| ATOM | 20751 | CA | VAL | F | 340 | 51.523 | 49.422 | −10.447 | 1.00 | 90.34 | F |
| ATOM | 20752 | CB | VAL | F | 340 | 52.342 | 48.918 | −11.643 | 1.00 | 97.64 | F |
| ATOM | 20753 | CG1 | VAL | F | 340 | 53.802 | 49.313 | −11.470 | 1.00 | 83.10 | F |
| ATOM | 20754 | CG2 | VAL | F | 340 | 52.205 | 47.417 | −11.771 | 1.00 | 83.10 | F |
| ATOM | 20755 | C | VAL | F | 340 | 51.653 | 50.933 | −10.402 | 1.00 | 92.91 | F |
| ATOM | 20756 | O | VAL | F | 340 | 51.208 | 51.607 | −11.316 | 1.00 | 89.76 | F |
| ATOM | 20757 | N | THR | F | 341 | 52.267 | 51.473 | −9.355 | 1.00 | 107.37 | F |
| ATOM | 20758 | CA | THR | F | 341 | 52.411 | 52.919 | −9.255 | 1.00 | 103.27 | F |
| ATOM | 20759 | CB | THR | F | 341 | 51.878 | 53.427 | −7.914 | 1.00 | 106.56 | F |
| ATOM | 20760 | OG1 | THR | F | 341 | 50.495 | 53.076 | −7.801 | 1.00 | 87.92 | F |
| ATOM | 20761 | CG2 | THR | F | 341 | 52.027 | 54.945 | −7.818 | 1.00 | 87.92 | F |
| ATOM | 20762 | C | THR | F | 341 | 53.849 | 53.383 | −9.420 | 1.00 | 105.42 | F |
| ATOM | 20763 | O | THR | F | 341 | 54.784 | 52.709 | −8.995 | 1.00 | 107.02 | F |
| ATOM | 20764 | N | GLY | F | 342 | 54.014 | 54.545 | −10.041 | 1.00 | 138.51 | F |
| ATOM | 20765 | CA | GLY | F | 342 | 55.343 | 55.081 | −10.252 | 1.00 | 142.68 | F |
| ATOM | 20766 | C | GLY | F | 342 | 55.644 | 56.286 | −9.383 | 1.00 | 141.65 | F |
| ATOM | 20767 | O | GLY | F | 342 | 55.836 | 57.386 | −9.896 | 1.00 | 135.92 | F |
| ATOM | 20768 | N | ASN | F | 343 | 55.682 | 56.084 | −8.068 | 1.00 | 139.48 | F |
| ATOM | 20769 | CA | ASN | F | 343 | 55.983 | 57.167 | −7.134 | 1.00 | 145.89 | F |
| ATOM | 20770 | CB | ASN | F | 343 | 55.769 | 56.705 | −5.690 | 1.00 | 173.65 | F |
| ATOM | 20771 | CG | ASN | F | 343 | 54.399 | 56.100 | −5.465 | 1.00 | 153.45 | F |
| ATOM | 20772 | OD1 | ASN | F | 343 | 53.376 | 56.767 | −5.628 | 1.00 | 153.45 | F |
| ATOM | 20773 | ND2 | ASN | F | 343 | 54.372 | 54.825 | −5.086 | 1.00 | 153.45 | F |
| ATOM | 20774 | C | ASN | F | 343 | 57.447 | 57.548 | −7.323 | 1.00 | 143.95 | F |
| ATOM | 20775 | O | ASN | F | 343 | 58.073 | 57.157 | −8.309 | 1.00 | 132.58 | F |
| ATOM | 20776 | N | GLU | F | 344 | 57.992 | 58.308 | −6.379 | 1.00 | 156.16 | F |
| ATOM | 20777 | CA | GLU | F | 344 | 59.392 | 58.717 | −6.440 | 1.00 | 156.16 | F |
| ATOM | 20778 | CB | GLU | F | 344 | 59.581 | 59.817 | −7.488 | 1.00 | 159.85 | F |
| ATOM | 20779 | CG | GLU | F | 344 | 59.858 | 59.250 | −8.875 | 1.00 | 139.75 | F |
| ATOM | 20780 | CD | GLU | F | 344 | 59.879 | 60.300 | −9.964 | 1.00 | 139.75 | F |
| ATOM | 20781 | OE1 | GLU | F | 344 | 58.797 | 60.793 | −10.343 | 1.00 | 139.75 | F |
| ATOM | 20782 | OE2 | GLU | F | 344 | 60.981 | 60.637 | −10.442 | 1.00 | 139.75 | F |
| ATOM | 20783 | C | GLU | F | 344 | 59.903 | 59.158 | −5.073 | 1.00 | 156.16 | F |
| ATOM | 20784 | O | GLU | F | 344 | 59.534 | 60.215 | −4.562 | 1.00 | 156.16 | F |
| ATOM | 20785 | N | LEU | F | 345 | 60.763 | 58.320 | −4.496 | 1.00 | 200.84 | F |
| ATOM | 20786 | CA | LEU | F | 345 | 61.330 | 58.537 | −3.166 | 1.00 | 200.84 | F |
| ATOM | 20787 | CB | LEU | F | 345 | 61.921 | 57.216 | −2.640 | 1.00 | 200.84 | F |
| ATOM | 20788 | CG | LEU | F | 345 | 60.964 | 56.022 | −2.470 | 1.00 | 155.35 | F |
| ATOM | 20789 | CD1 | LEU | F | 345 | 61.717 | 54.833 | −1.886 | 1.00 | 155.35 | F |
| ATOM | 20790 | CD2 | LEU | F | 345 | 59.805 | 56.405 | −1.559 | 1.00 | 155.35 | F |
| ATOM | 20791 | C | LEU | F | 345 | 62.353 | 59.670 | −2.996 | 1.00 | 200.84 | F |
| ATOM | 20792 | O | LEU | F | 345 | 63.283 | 59.560 | −2.196 | 1.00 | 200.84 | F |
| ATOM | 20793 | N | GLU | F | 346 | 62.177 | 60.750 | −3.753 | 1.00 | 168.95 | F |
| ATOM | 20794 | CA | GLU | F | 346 | 63.029 | 61.941 | −3.666 | 1.00 | 168.95 | F |
| ATOM | 20795 | CB | GLU | F | 346 | 62.896 | 62.536 | −2.262 | 1.00 | 188.72 | F |
| ATOM | 20796 | CG | GLU | F | 346 | 61.453 | 62.539 | −1.771 | 1.00 | 174.15 | F |
| ATOM | 20797 | CD | GLU | F | 346 | 61.207 | 63.511 | −0.638 | 1.00 | 174.15 | F |
| ATOM | 20798 | OE1 | GLU | F | 346 | 61.312 | 64.733 | −0.876 | 1.00 | 174.15 | F |
| ATOM | 20799 | OE2 | GLU | F | 346 | 60.907 | 63.056 | 0.486 | 1.00 | 174.15 | F |
| ATOM | 20800 | C | GLU | F | 346 | 64.512 | 61.864 | −4.055 | 1.00 | 168.95 | F |
| ATOM | 20801 | O | GLU | F | 346 | 64.898 | 62.369 | −5.109 | 1.00 | 168.95 | F |
| ATOM | 20802 | N | ASP | F | 347 | 65.347 | 61.257 | −3.213 | 1.00 | 187.28 | F |
| ATOM | 20803 | CA | ASP | F | 347 | 66.777 | 61.172 | −3.515 | 1.00 | 187.28 | F |
| ATOM | 20804 | CB | ASP | F | 347 | 67.543 | 60.531 | −2.348 | 1.00 | 200.84 | F |
| ATOM | 20805 | CG | ASP | F | 347 | 67.496 | 59.013 | −2.373 | 1.00 | 200.84 | F |
| ATOM | 20806 | OD1 | ASP | F | 347 | 66.387 | 58.446 | −2.278 | 1.00 | 200.84 | F |
| ATOM | 20807 | OD2 | ASP | F | 347 | 68.574 | 58.387 | −2.488 | 1.00 | 200.84 | F |
| ATOM | 20808 | C | ASP | F | 347 | 67.062 | 60.400 | −4.803 | 1.00 | 187.28 | F |
| ATOM | 20809 | O | ASP | F | 347 | 67.969 | 60.755 | −5.558 | 1.00 | 187.28 | F |
| ATOM | 20810 | N | GLY | F | 348 | 66.288 | 59.346 | −5.049 | 1.00 | 148.80 | F |
| ATOM | 20811 | CA | GLY | F | 348 | 66.473 | 58.549 | −6.249 | 1.00 | 146.11 | F |
| ATOM | 20812 | C | GLY | F | 348 | 65.490 | 58.950 | −7.329 | 1.00 | 143.07 | F |
| ATOM | 20813 | O | GLY | F | 348 | 64.374 | 59.372 | −7.029 | 1.00 | 138.08 | F |
| ATOM | 20814 | N | ASN | F | 349 | 65.894 | 58.817 | −8.588 | 1.00 | 200.77 | F |
| ATOM | 20815 | CA | ASN | F | 349 | 65.026 | 59.189 | −9.695 | 1.00 | 200.67 | F |
| ATOM | 20816 | CB | ASN | F | 349 | 65.664 | 58.785 | −11.033 | 1.00 | 200.84 | F |
| ATOM | 20817 | CG | ASN | F | 349 | 66.437 | 57.485 | −10.948 | 1.00 | 188.18 | F |
| ATOM | 20818 | OD1 | ASN | F | 349 | 67.418 | 57.376 | −10.209 | 1.00 | 188.18 | F |
| ATOM | 20819 | ND2 | ASN | F | 349 | 66.003 | 56.492 | −11.714 | 1.00 | 188.18 | F |
| ATOM | 20820 | C | ASN | F | 349 | 63.597 | 58.646 | −9.592 | 1.00 | 200.77 | F |
| ATOM | 20821 | O | ASN | F | 349 | 62.709 | 59.348 | −9.111 | 1.00 | 200.77 | F |
| ATOM | 20822 | N | MET | F | 350 | 63.371 | 57.406 | −10.019 | 1.00 | 140.67 | F |
| ATOM | 20823 | CA | MET | F | 350 | 62.025 | 56.832 | −9.981 | 1.00 | 136.76 | F |
| ATOM | 20824 | CB | MET | F | 350 | 61.550 | 56.562 | −11.411 | 1.00 | 200.84 | F |
| ATOM | 20825 | CG | MET | F | 350 | 60.166 | 55.954 | −11.506 | 1.00 | 160.28 | F |
| ATOM | 20826 | SD | MET | F | 350 | 59.607 | 55.782 | −13.203 | 1.00 | 160.28 | F |
| ATOM | 20827 | CE | MET | F | 350 | 58.490 | 57.199 | −13.330 | 1.00 | 160.28 | F |
| ATOM | 20828 | C | MET | F | 350 | 61.876 | 55.559 | −9.144 | 1.00 | 133.90 | F |
| ATOM | 20829 | O | MET | F | 350 | 62.846 | 54.834 | −8.916 | 1.00 | 130.33 | F |
| ATOM | 20830 | N | THR | F | 351 | 60.650 | 55.299 | −8.688 | 1.00 | 128.39 | F |
| ATOM | 20831 | CA | THR | F | 351 | 60.348 | 54.113 | −7.886 | 1.00 | 126.35 | F |
| ATOM | 20832 | CB | THR | F | 351 | 60.202 | 54.460 | −6.374 | 1.00 | 133.23 | F |
| ATOM | 20833 | OG1 | THR | F | 351 | 60.658 | 53.358 | −5.583 | 1.00 | 100.35 | F |
| ATOM | 20834 | CG2 | THR | F | 351 | 58.744 | 54.722 | −6.005 | 1.00 | 100.35 | F |
| ATOM | 20835 | C | THR | F | 351 | 59.047 | 53.465 | −8.369 | 1.00 | 120.58 | F |
| ATOM | 20836 | O | THR | F | 351 | 58.054 | 54.154 | −8.621 | 1.00 | 112.17 | F |
| ATOM | 20837 | N | LEU | F | 352 | 59.058 | 52.143 | −8.508 | 1.00 | 94.95 | F |
| ATOM | 20838 | CA | LEU | F | 352 | 57.871 | 51.424 | −8.950 | 1.00 | 97.97 | F |
| ATOM | 20839 | CB | LEU | F | 352 | 58.196 | 50.555 | −10.165 | 1.00 | 141.26 | F |
| ATOM | 20840 | CG | LEU | F | 352 | 58.233 | 51.374 | −11.459 | 1.00 | 122.32 | F |
| ATOM | 20541 | CD1 | LEU | F | 352 | 58.825 | 50.563 | −12.596 | 1.00 | 122.32 | F |
| ATOM | 20842 | CD2 | LEU | F | 352 | 56.819 | 51.827 | −11.789 | 1.00 | 122.32 | F |
| ATOM | 20843 | C | LEU | F | 352 | 57.304 | 50.582 | −7.822 | 1.00 | 94.65 | F |
| ATOM | 20844 | O | LEU | F | 352 | 58.041 | 49.918 | −7.092 | 1.00 | 90.27 | F |
| ATOM | 20845 | N | GLU | F | 353 | 55.984 | 50.625 | −7.684 | 1.00 | 84.15 | F |
| ATOM | 20846 | CA | GLU | F | 353 | 55.306 | 49.890 | −6.635 | 1.00 | 80.40 | F |
| ATOM | 20847 | CB | GLU | F | 353 | 54.767 | 50.861 | −5.595 | 1.00 | 200.84 | F |
| ATOM | 20848 | CG | GLU | F | 353 | 54.069 | 50.189 | −4.443 | 1.00 | 163.89 | F |
| ATOM | 20849 | CD | GLU | F | 353 | 53.480 | 51.190 | −3.482 | 1.00 | 163.89 | F |
| ATOM | 20850 | OE1 | GLU | F | 353 | 54.241 | 52.043 | −2.976 | 1.00 | 163.89 | F |
| ATOM | 20851 | OE2 | GLU | F | 353 | 52.259 | 51.124 | −3.234 | 1.00 | 163.89 | F |
| ATOM | 20852 | C | GLU | F | 353 | 54.166 | 49.053 | −7.178 | 1.00 | 83.65 | F |
| ATOM | 20853 | O | GLU | F | 353 | 53.177 | 49.581 | −7.684 | 1.00 | 84.43 | F |
| ATOM | 20854 | N | CYS | F | 354 | 54.316 | 47.740 | −7.061 | 1.00 | 92.98 | F |
| ATOM | 20855 | CA | CYS | F | 354 | 53.313 | 46.795 | −7.527 | 1.00 | 93.26 | F |
| ATOM | 20856 | C | CYS | F | 354 | 52.513 | 46.394 | −6.305 | 1.00 | 94.35 | F |
| ATOM | 20857 | O | CYS | F | 354 | 53.081 | 45.906 | −5.328 | 1.00 | 93.05 | F |
| ATOM | 20858 | CB | CYS | F | 354 | 54.001 | 45.562 | −8.116 | 1.00 | 148.13 | F |
| ATOM | 20859 | SG | CYS | F | 354 | 52.961 | 44.456 | −9.125 | 1.00 | 125.32 | F |
| ATOM | 20860 | N | THR | F | 355 | 51.206 | 46.616 | −6.333 | 1.00 | 72.21 | F |
| ATOM | 20861 | CA | THR | F | 355 | 50.398 | 46.230 | −5.184 | 1.00 | 61.57 | F |
| ATOM | 20862 | CB | THR | F | 355 | 49.752 | 47.444 | −4.487 | 1.00 | 146.93 | F |
| ATOM | 20863 | OG1 | THR | F | 355 | 48.384 | 47.557 | −4.896 | 1.00 | 96.08 | F |
| ATOM | 20864 | CG2 | THR | F | 355 | 50.497 | 48.722 | −4.840 | 1.00 | 96.08 | F |
| ATOM | 20865 | C | THR | F | 355 | 49.304 | 45.270 | −5.616 | 1.00 | 75.06 | F |
| ATOM | 20866 | O | THR | F | 355 | 48.537 | 45.541 | −6.547 | 1.00 | 67.86 | F |
| ATOM | 20867 | N | VAL | F | 356 | 49.250 | 44.138 | −4.926 | 1.00 | 88.62 | F |
| ATOM | 20868 | CA | VAL | F | 356 | 48.278 | 43.093 | −5.206 | 1.00 | 84.34 | F |
| ATOM | 20869 | CB | VAL | F | 356 | 48.994 | 41.739 | −5.345 | 1.00 | 80.43 | F |
| ATOM | 20870 | CG1 | VAL | F | 356 | 48.038 | 40.685 | −5.871 | 1.00 | 64.96 | F |
| ATOM | 20871 | CG2 | VAL | F | 356 | 50.194 | 41.901 | −6.276 | 1.00 | 64.96 | F |
| ATOM | 20872 | C | VAL | F | 356 | 47.295 | 43.070 | −4.047 | 1.00 | 93.18 | F |
| ATOM | 20873 | O | VAL | F | 356 | 47.679 | 42.853 | −2.896 | 1.00 | 94.29 | F |
| ATOM | 20874 | N | ASN | F | 357 | 46.022 | 43.294 | −4.360 | 1.00 | 82.80 | F |
| ATOM | 20875 | CA | ASN | F | 357 | 44.995 | 43.362 | −3.330 | 1.00 | 79.02 | F |
| ATOM | 20876 | CB | ASN | F | 357 | 44.144 | 44.610 | −3.543 | 1.00 | 184.68 | F |
| ATOM | 20877 | CG | ASN | F | 357 | 44.944 | 45.879 | −3.387 | 1.00 | 138.60 | F |
| ATOM | 20878 | OD1 | ASN | F | 357 | 45.595 | 46.087 | −2.365 | 1.00 | 138.60 | F |
| ATOM | 20879 | ND2 | ASN | F | 357 | 44.905 | 46.736 | −4.401 | 1.00 | 138.60 | F |
| ATOM | 20880 | C | ASN | F | 357 | 44.084 | 42.172 | −3.123 | 1.00 | 83.18 | F |
| ATOM | 20881 | O | ASN | F | 357 | 43.456 | 41.665 | −4.050 | 1.00 | 86.06 | F |
| ATOM | 20882 | N | SER | F | 358 | 44.013 | 41.773 | −1.858 | 1.00 | 87.98 | F |
| ATOM | 20883 | CA | SER | F | 358 | 43.208 | 40.662 | −1.377 | 1.00 | 95.28 | F |
| ATOM | 20884 | CB | SER | F | 358 | 41.827 | 41.164 | −0.942 | 1.00 | 87.42 | F |
| ATOM | 20885 | OG | SER | F | 358 | 41.078 | 41.621 | −2.049 | 1.00 | 85.76 | F |
| ATOM | 20886 | C | SER | F | 358 | 43.064 | 39.466 | −2.317 | 1.00 | 100.00 | F |
| ATOM | 20887 | O | SER | F | 358 | 42.135 | 39.389 | −3.130 | 1.00 | 95.55 | F |
| ATOM | 20888 | N | PHE | F | 359 | 43.998 | 38.527 | −2.159 | 1.00 | 85.09 | F |
| ATOM | 20889 | CA | PHE | F | 359 | 44.064 | 37.277 | −2.922 | 1.00 | 79.40 | F |
| ATOM | 20890 | CB | PHE | F | 359 | 45.371 | 37.244 | −3.708 | 1.00 | 84.97 | F |
| ATOM | 20891 | CG | PHE | F | 359 | 46.583 | 37.643 | −2.900 | 1.00 | 79.57 | F |
| ATOM | 20892 | CD1 | PHE | F | 359 | 47.225 | 36.722 | −2.073 | 1.00 | 79.57 | F |
| ATOM | 20893 | CD2 | PHE | F | 359 | 47.098 | 38.938 | −2.988 | 1.00 | 79.57 | F |
| ATOM | 20894 | CE1 | PHE | F | 359 | 48.366 | 37.082 | −1.347 | 1.00 | 79.57 | F |
| ATOM | 20895 | CE2 | PHE | F | 359 | 48.244 | 39.313 | −2.264 | 1.00 | 79.57 | F |
| ATOM | 20896 | CZ | PHE | F | 359 | 48.878 | 38.383 | −1.444 | 1.00 | 79.57 | F |
| ATOM | 20897 | C | PHE | F | 359 | 44.030 | 36.144 | −1.895 | 1.00 | 85.99 | F |
| ATOM | 20898 | O | PHE | F | 359 | 44.575 | 36.318 | −0.809 | 1.00 | 83.54 | F |
| ATOM | 20899 | N | TYR | F | 360 | 43.410 | 34.997 | −2.180 | 1.00 | 128.41 | F |
| ATOM | 20900 | CA | TYR | F | 360 | 43.429 | 34.003 | −1.119 | 1.00 | 128.19 | F |
| ATOM | 20901 | CB | TYR | F | 360 | 42.275 | 32.999 | −1.125 | 1.00 | 200.84 | F |
| ATOM | 20902 | CG | TYR | F | 360 | 42.594 | 31.931 | −0.074 | 1.00 | 113.61 | F |
| ATOM | 20903 | CD1 | TYR | F | 360 | 43.043 | 32.305 | 1.203 | 1.00 | 113.61 | F |
| ATOM | 20904 | CE1 | TYR | F | 360 | 43.529 | 31.363 | 2.117 | 1.00 | 113.61 | F |
| ATOM | 20905 | CD2 | TYR | F | 360 | 42.624 | 30.574 | −0.395 | 1.00 | 113.61 | F |
| ATOM | 20906 | CE2 | TYR | F | 360 | 43.105 | 29.619 | 0.524 | 1.00 | 113.61 | F |
| ATOM | 20907 | CZ | TYR | F | 360 | 43.558 | 30.025 | 1.770 | 1.00 | 113.61 | F |
| ATOM | 20908 | OH | TYR | F | 360 | 44.047 | 29.098 | 2.663 | 1.00 | 113.61 | F |
| ATOM | 20909 | C | TYR | F | 360 | 44.683 | 33.187 | −0.913 | 1.00 | 129.28 | F |
| ATOM | 20910 | O | TYR | F | 360 | 45.465 | 33.515 | −0.031 | 1.00 | 135.21 | F |
| ATOM | 20911 | N | PRO | F | 361 | 44.882 | 32.094 | −1.680 | 1.00 | 73.77 | F |
| ATOM | 20912 | CD | PRO | F | 361 | 44.398 | 31.769 | −3.035 | 1.00 | 76.87 | F |
| ATOM | 20913 | CA | PRO | F | 361 | 46.131 | 31.385 | −1.363 | 1.00 | 69.36 | F |
| ATOM | 20914 | CB | PRO | F | 361 | 46.330 | 30.455 | −2.566 | 1.00 | 76.11 | F |
| ATOM | 20915 | CG | PRO | F | 361 | 45.622 | 31.151 | −3.673 | 1.00 | 76.87 | F |
| ATOM | 20916 | C | PRO | F | 361 | 47.198 | 32.460 | −1.169 | 1.00 | 76.76 | F |
| ATOM | 20917 | O | PRO | F | 361 | 47.560 | 33.174 | −2.097 | 1.00 | 73.93 | F |
| ATOM | 20918 | N | PRO | F | 362 | 47.664 | 32.622 | 0.078 | 1.00 | 62.55 | F |
| ATOM | 20919 | CD | PRO | F | 362 | 47.333 | 31.706 | 1.183 | 1.00 | 69.12 | F |
| ATOM | 20920 | CA | PRO | F | 362 | 48.668 | 33.609 | 0.498 | 1.00 | 56.26 | F |
| ATOM | 20921 | CB | PRO | F | 362 | 48.804 | 33.351 | 1.992 | 1.00 | 69.12 | F |
| ATOM | 20922 | CG | PRO | F | 362 | 48.494 | 31.897 | 2.108 | 1.00 | 69.12 | F |
| ATOM | 20923 | C | PRO | F | 362 | 50.006 | 33.658 | −0.208 | 1.00 | 58.86 | F |
| ATOM | 20924 | O | PRO | F | 362 | 50.747 | 34.643 | −0.068 | 1.00 | 63.62 | F |
| ATOM | 20925 | N | ASP | F | 363 | 50.324 | 32.608 | −0.963 | 1.00 | 60.73 | F |
| ATOM | 20926 | CA | ASP | F | 363 | 51.580 | 32.580 | −1.701 | 1.00 | 60.58 | F |
| ATOM | 20927 | CB | ASP | F | 363 | 52.001 | 31.134 | −2.032 | 1.00 | 88.03 | F |
| ATOM | 20928 | CG | ASP | F | 363 | 50.867 | 30.291 | −2.607 | 1.00 | 82.24 | F |
| ATOM | 20929 | OD1 | ASP | F | 363 | 49.818 | 30.152 | −1.934 | 1.00 | 82.24 | F |
| ATOM | 20930 | OD2 | ASP | F | 363 | 51.037 | 29.751 | −3.729 | 1.00 | 82.24 | F |
| ATOM | 20931 | C | ASP | F | 363 | 51.460 | 33.408 | −2.970 | 1.00 | 64.12 | F |
| ATOM | 20932 | O | ASP | F | 363 | 50.565 | 33.204 | −3.784 | 1.00 | 60.62 | F |
| ATOM | 20933 | N | VAL | F | 364 | 52.351 | 34.376 | −3.109 | 1.00 | 53.71 | F |
| ATOM | 20934 | CA | VAL | F | 364 | 52.366 | 35.233 | −4.285 | 1.00 | 56.03 | F |
| ATOM | 20935 | CB | VAL | F | 364 | 51.609 | 36.567 | −4.057 | 1.00 | 72.46 | F |
| ATOM | 20936 | CG1 | VAL | F | 364 | 52.062 | 37.600 | −5.057 | 1.00 | 50.85 | F |
| ATOM | 20937 | CG2 | VAL | F | 364 | 50.130 | 36.357 | −4.245 | 1.00 | 50.85 | F |
| ATOM | 20938 | C | VAL | F | 364 | 53.808 | 35.551 | −4.584 | 1.00 | 63.37 | F |
| ATOM | 20939 | O | VAL | F | 364 | 54.613 | 35.750 | −3.677 | 1.00 | 55.11 | F |
| ATOM | 20940 | N | ILE | F | 365 | 54.144 | 35.572 | −5.860 | 1.00 | 69.91 | F |
| ATOM | 20941 | CA | ILE | F | 365 | 55.494 | 35.898 | −6.246 | 1.00 | 63.96 | F |
| ATOM | 20942 | CB | ILE | F | 365 | 56.271 | 34.664 | −6.714 | 1.00 | 70.32 | F |
| ATOM | 20943 | CG2 | ILE | F | 365 | 57.627 | 35.085 | −7.265 | 1.00 | 46.76 | F |
| ATOM | 20944 | CG1 | ILE | F | 365 | 56.461 | 33.715 | −5.527 | 1.00 | 46.76 | F |
| ATOM | 20945 | CD1 | ILE | F | 365 | 57.273 | 32.475 | −5.827 | 1.00 | 46.76 | F |
| ATOM | 20946 | C | ILE | F | 365 | 55.429 | 36.942 | −7.333 | 1.00 | 70.72 | F |
| ATOM | 20947 | O | ILE | F | 365 | 54.521 | 36.954 | −8.169 | 1.00 | 71.49 | F |
| ATOM | 20948 | N | THR | F | 366 | 56.409 | 37.826 | −7.309 | 1.00 | 54.79 | F |
| ATOM | 20949 | CA | THR | F | 366 | 56.462 | 38.923 | −8.241 | 1.00 | 63.07 | F |
| ATOM | 20950 | CB | THR | F | 366 | 56.222 | 40.225 | −7.468 | 1.00 | 112.43 | F |
| ATOM | 20951 | OG1 | THR | F | 366 | 55.093 | 40.901 | −8.024 | 1.00 | 100.11 | F |
| ATOM | 20952 | CG2 | THR | F | 366 | 57.453 | 41.121 | −7.500 | 1.00 | 100.11 | F |
| ATOM | 20953 | C | THR | F | 366 | 57.783 | 38.997 | −8.985 | 1.00 | 72.05 | F |
| ATOM | 20954 | O | THR | F | 366 | 58.853 | 38.839 | −8.391 | 1.00 | 71.29 | F |
| ATOM | 20955 | N | LYS | F | 367 | 57.698 | 39.239 | −10.289 | 1.00 | 75.08 | F |
| ATOM | 20956 | CA | LYS | F | 367 | 58.888 | 39.360 | −11.121 | 1.00 | 78.28 | F |
| ATOM | 20957 | CB | LYS | F | 367 | 58.976 | 38.216 | −12.135 | 1.00 | 96.17 | F |
| ATOM | 20958 | CG | LYS | F | 367 | 59.354 | 36.886 | −11.530 | 1.00 | 87.18 | F |
| ATOM | 20959 | CD | LYS | F | 367 | 59.704 | 35.864 | −12.591 | 1.00 | 87.18 | F |
| ATOM | 20960 | CE | LYS | F | 367 | 60.269 | 34.614 | −11.944 | 1.00 | 87.18 | F |
| ATOM | 20961 | NZ | LYS | F | 367 | 60.889 | 33.704 | −12.943 | 1.00 | 87.18 | F |
| ATOM | 20962 | C | LYS | F | 367 | 58.869 | 40.680 | −11.867 | 1.00 | 80.98 | F |
| ATOM | 20963 | O | LYS | F | 367 | 57.863 | 41.034 | −12.482 | 1.00 | 81.29 | F |
| ATOM | 20964 | N | TRP | F | 368 | 59.978 | 41.409 | −11.796 | 1.00 | 75.36 | F |
| ATOM | 20965 | CA | TRP | F | 368 | 60.100 | 42.680 | −12.498 | 1.00 | 75.42 | F |
| ATOM | 20966 | CB | TRP | F | 368 | 60.740 | 43.742 | −11.602 | 1.00 | 125.50 | F |
| ATOM | 20967 | CG | TRP | F | 368 | 59.844 | 44.176 | −10.494 | 1.00 | 105.42 | F |
| ATOM | 20968 | CD2 | TRP | F | 368 | 58.847 | 45.203 | −10.551 | 1.00 | 105.42 | F |
| ATOM | 20969 | CE2 | TRP | F | 368 | 58.198 | 45.227 | −9.298 | 1.00 | 105.42 | F |
| ATOM | 20970 | CE3 | TRP | F | 368 | 58.437 | 46.102 | −11.541 | 1.00 | 105.42 | F |
| ATOM | 20971 | CD1 | TRP | F | 368 | 59.765 | 43.636 | −9.244 | 1.00 | 105.42 | F |
| ATOM | 20972 | NE1 | TRP | F | 368 | 58.779 | 44.262 | −8.518 | 1.00 | 105.42 | F |
| ATOM | 20973 | CZ2 | TRP | F | 368 | 57.165 | 46.115 | −9.008 | 1.00 | 105.42 | F |
| ATOM | 20974 | CZ3 | TRP | F | 368 | 57.408 | 46.985 | −11.252 | 1.00 | 105.42 | F |
| ATOM | 20975 | CH2 | TRP | F | 368 | 56.784 | 46.984 | −9.994 | 1.00 | 105.42 | F |
| ATOM | 20976 | C | TRP | F | 368 | 60.946 | 42.476 | −13.748 | 1.00 | 77.37 | F |
| ATOM | 20977 | O | TRP | F | 368 | 62.126 | 42.140 | −13.665 | 1.00 | 77.56 | F |
| ATOM | 20978 | N | ILE | F | 369 | 60.337 | 42.690 | −14.905 | 1.00 | 78.42 | F |
| ATOM | 20979 | CA | ILE | F | 369 | 61.026 | 42.493 | −16.167 | 1.00 | 79.79 | F |
| ATOM | 20980 | CB | ILE | F | 369 | 60.411 | 41.298 | −16.916 | 1.00 | 119.04 | F |
| ATOM | 20981 | CG2 | ILE | F | 369 | 61.183 | 41.027 | −18.191 | 1.00 | 96.72 | F |
| ATOM | 20982 | CG1 | ILE | F | 369 | 60.417 | 40.067 | −16.011 | 1.00 | 96.72 | F |
| ATOM | 20983 | CD1 | ILE | F | 369 | 59.681 | 38.887 | −16.590 | 1.00 | 96.72 | F |
| ATOM | 20984 | C | ILE | F | 369 | 60.930 | 43.728 | −17.056 | 1.00 | 81.61 | F |
| ATOM | 20985 | O | ILE | F | 369 | 59.859 | 44.317 | −17.196 | 1.00 | 78.41 | F |
| ATOM | 20986 | N | GLU | F | 370 | 62.049 | 44.120 | −17.657 | 1.00 | 92.63 | F |
| ATOM | 20987 | CA | GLU | F | 370 | 62.049 | 45.272 | −18.547 | 1.00 | 95.28 | F |
| ATOM | 20988 | CB | GLU | F | 370 | 63.462 | 45.514 | −19.073 | 1.00 | 138.77 | F |
| ATOM | 20989 | CG | GLU | F | 370 | 64.356 | 46.167 | −18.032 | 1.00 | 117.41 | F |
| ATOM | 20990 | CD | GLU | F | 370 | 65.820 | 46.190 | −18.423 | 1.00 | 117.41 | F |
| ATOM | 20991 | OE1 | GLU | F | 370 | 66.136 | 46.589 | −19.563 | 1.00 | 117.41 | F |
| ATOM | 20992 | OE2 | GLU | F | 370 | 66.660 | 45.816 | −17.578 | 1.00 | 117.41 | F |
| ATOM | 20993 | C | GLU | F | 370 | 61.064 | 45.007 | −19.686 | 1.00 | 95.46 | F |
| ATOM | 20994 | O | GLU | F | 370 | 61.083 | 43.940 | −20.295 | 1.00 | 94.31 | F |
| ATOM | 20995 | N | SER | F | 371 | 60.195 | 45.975 | −19.963 | 1.00 | 98.01 | F |
| ATOM | 20996 | CA | SER | F | 371 | 59.171 | 45.832 | −21.001 | 1.00 | 98.55 | F |
| ATOM | 20997 | CB | SER | F | 371 | 58.318 | 47.096 | −21.064 | 1.00 | 99.16 | F |
| ATOM | 20998 | OG | SER | F | 371 | 59.122 | 48.216 | −21.371 | 1.00 | 88.24 | F |
| ATOM | 20999 | C | SER | F | 371 | 59.660 | 45.492 | −22.410 | 1.00 | 97.82 | F |
| ATOM | 21000 | O | SER | F | 371 | 58.896 | 44.954 | −23.215 | 1.00 | 98.76 | F |
| ATOM | 21001 | N | GLU | F | 372 | 60.915 | 45.808 | −22.716 | 1.00 | 92.77 | F |
| ATOM | 21002 | CA | GLU | F | 372 | 61.458 | 45.506 | −24.035 | 1.00 | 91.32 | F |
| ATOM | 21003 | CB | GLU | F | 372 | 62.938 | 45.890 | −24.104 | 1.00 | 127.65 | F |
| ATOM | 21004 | CG | GLU | F | 372 | 63.801 | 45.201 | −23.061 | 1.00 | 127.65 | F |
| ATOM | 21005 | CD | GLU | F | 372 | 65.282 | 45.333 | −23.346 | 1.00 | 127.65 | F |
| ATOM | 21006 | OE1 | GLU | F | 372 | 65.727 | 44.818 | −24.394 | 1.00 | 127.65 | F |
| ATOM | 21007 | OE2 | GLU | F | 372 | 66.000 | 45.947 | −22.526 | 1.00 | 127.65 | F |
| ATOM | 21008 | C | GLU | F | 372 | 61.314 | 44.015 | −24.341 | 1.00 | 95.93 | F |
| ATOM | 21009 | O | GLU | F | 372 | 60.997 | 43.626 | −25.468 | 1.00 | 97.00 | F |
| ATOM | 21010 | N | HIS | F | 373 | 61.538 | 43.191 | −23.320 | 1.00 | 110.62 | F |
| ATOM | 21011 | CA | HIS | F | 373 | 61.462 | 41.744 | −23.453 | 1.00 | 114.11 | F |
| ATOM | 21012 | CB | HIS | F | 373 | 61.961 | 41.081 | −22.176 | 1.00 | 133.60 | F |
| ATOM | 21013 | CG | HIS | F | 373 | 63.389 | 41.393 | −21.864 | 1.00 | 102.95 | F |
| ATOM | 21014 | CD2 | HIS | F | 373 | 63.970 | 41.915 | −20.758 | 1.00 | 102.95 | F |
| ATOM | 21015 | ND1 | HIS | F | 373 | 64.409 | 41.171 | −22.764 | 1.00 | 102.95 | F |
| ATOM | 21016 | CE1 | HIS | F | 373 | 65.557 | 41.542 | −22.226 | 1.00 | 102.95 | F |
| ATOM | 21017 | NE2 | HIS | F | 373 | 65.318 | 41.998 | −21.009 | 1.00 | 102.95 | F |
| ATOM | 21018 | C | HIS | F | 373 | 60.097 | 41.191 | −23.808 | 1.00 | 109.88 | F |
| ATOM | 21019 | O | HIS | F | 373 | 59.973 | 40.003 | −24.092 | 1.00 | 105.72 | F |
| ATOM | 21020 | N | PHE | F | 374 | 59.069 | 42.029 | −23.790 | 1.00 | 82.14 | F |
| ATOM | 21021 | CA | PHE | F | 374 | 57.746 | 41.549 | −24.149 | 1.00 | 82.91 | F |
| ATOM | 21022 | CB | PHE | F | 374 | 56.685 | 42.104 | −23.207 | 1.00 | 109.56 | F |
| ATOM | 21023 | CG | PHE | F | 374 | 56.731 | 41.493 | −21.850 | 1.00 | 89.74 | F |
| ATOM | 21024 | CD1 | PHE | F | 374 | 57.647 | 41.941 | −20.906 | 1.00 | 89.74 | F |
| ATOM | 21025 | CD2 | PHE | F | 374 | 55.911 | 40.417 | −21.535 | 1.00 | 89.74 | F |
| ATOM | 21026 | CE1 | PHE | F | 374 | 57.751 | 41.327 | −19.669 | 1.00 | 89.74 | F |
| ATOM | 21027 | CE2 | PHE | F | 374 | 56.006 | 39.793 | −20.301 | 1.00 | 89.74 | F |
| ATOM | 21028 | CZ | PHE | F | 374 | 56.931 | 40.250 | −19.364 | 1.00 | 89.74 | F |
| ATOM | 21029 | C | PHE | F | 374 | 57.393 | 41.884 | −25.585 | 1.00 | 78.99 | F |
| ATOM | 21030 | O | PHE | F | 374 | 56.240 | 41.740 | −25.993 | 1.00 | 77.23 | F |
| ATOM | 21031 | N | LYS | F | 375 | 58.401 | 42.316 | −26.344 | 1.00 | 115.84 | F |
| ATOM | 21032 | CA | LYS | F | 375 | 58.250 | 42.669 | −27.757 | 1.00 | 118.47 | F |
| ATOM | 21033 | CB | LYS | F | 375 | 58.713 | 41.506 | −28.651 | 1.00 | 126.83 | F |
| ATOM | 21034 | CG | LYS | F | 375 | 60.225 | 41.377 | −28.843 | 1.00 | 100.67 | F |
| ATOM | 21035 | CD | LYS | F | 375 | 60.947 | 40.915 | −27.586 | 1.00 | 100.67 | F |
| ATOM | 21036 | CE | LYS | F | 375 | 62.432 | 40.697 | −27.852 | 1.00 | 100.67 | F |
| ATOM | 21037 | NZ | LYS | F | 375 | 63.179 | 40.304 | −26.620 | 1.00 | 100.67 | F |
| ATOM | 21038 | C | LYS | F | 375 | 56.836 | 43.075 | −28.169 | 1.00 | 123.49 | F |
| ATOM | 21039 | O | LYS | F | 375 | 56.270 | 42.503 | −29.098 | 1.00 | 125.75 | F |
| ATOM | 21040 | N | GLY | F | 376 | 56.260 | 44.050 | −27.475 | 1.00 | 79.05 | F |
| ATOM | 21041 | CA | GLY | F | 376 | 54.924 | 44.502 | −27.830 | 1.00 | 79.05 | F |
| ATOM | 21042 | C | GLY | F | 376 | 53.731 | 43.764 | −27.240 | 1.00 | 79.05 | F |
| ATOM | 21043 | O | GLY | F | 376 | 52.682 | 44.373 | −27.020 | 1.00 | 79.05 | F |
| ATOM | 21044 | N | GLU | F | 377 | 53.863 | 42.464 | −26.995 | 1.00 | 114.85 | F |
| ATOM | 21045 | CA | GLU | F | 377 | 52.761 | 41.690 | −26.426 | 1.00 | 114.85 | F |
| ATOM | 21046 | CB | GLU | F | 377 | 52.626 | 40.346 | −27.142 | 1.00 | 126.54 | F |
| ATOM | 21047 | CG | GLU | F | 377 | 51.350 | 39.596 | −26.796 | 1.00 | 126.54 | F |
| ATOM | 21048 | CD | GLU | F | 377 | 51.222 | 38.287 | −27.549 | 1.00 | 126.54 | F |
| ATOM | 21049 | OE1 | GLU | F | 377 | 50.147 | 37.657 | −27.459 | 1.00 | 126.54 | F |
| ATOM | 21050 | OE2 | GLU | F | 377 | 52.195 | 37.888 | −28.228 | 1.00 | 126.54 | F |
| ATOM | 21051 | C | GLU | F | 377 | 53.006 | 41.462 | −24.937 | 1.00 | 114.85 | F |
| ATOM | 21052 | O | GLU | F | 377 | 53.989 | 40.827 | −24.552 | 1.00 | 114.85 | F |
| ATOM | 21053 | N | TYR | F | 378 | 52.108 | 41.979 | −24.100 | 1.00 | 82.59 | F |
| ATOM | 21054 | CA | TYR | F | 378 | 52.256 | 41.845 | −22.657 | 1.00 | 82.59 | F |
| ATOM | 21055 | CB | TYR | F | 378 | 51.756 | 43.111 | −21.962 | 1.00 | 84.77 | F |
| ATOM | 21056 | CG | TYR | F | 378 | 52.659 | 44.292 | −22.223 | 1.00 | 84.77 | F |
| ATOM | 21057 | CD1 | TYR | F | 378 | 52.406 | 45.179 | −23.270 | 1.00 | 84.77 | F |
| ATOM | 21058 | CE1 | TYR | F | 378 | 53.297 | 46.213 | −23.568 | 1.00 | 84.77 | F |
| ATOM | 21059 | CD2 | TYR | F | 378 | 53.823 | 44.472 | −21.476 | 1.00 | 84.77 | F |
| ATOM | 21060 | CE2 | TYR | F | 378 | 54.718 | 45.500 | −21.768 | 1.00 | 84.77 | F |
| ATOM | 21061 | CZ | TYR | F | 378 | 54.453 | 46.364 | −22.813 | 1.00 | 84.77 | F |
| ATOM | 21062 | OH | TYR | F | 378 | 55.358 | 47.359 | −23.113 | 1.00 | 84.77 | F |
| ATOM | 21063 | C | TYR | F | 378 | 51.623 | 40.614 | −22.034 | 1.00 | 82.59 | F |
| ATOM | 21064 | O | TYR | F | 378 | 50.676 | 40.705 | −21.256 | 1.00 | 82.59 | F |
| ATOM | 21065 | N | LYS | F | 379 | 52.171 | 39.461 | −22.400 | 1.00 | 77.68 | F |
| ATOM | 21066 | CA | LYS | F | 379 | 51.746 | 38.167 | −21.886 | 1.00 | 77.68 | F |
| ATOM | 21067 | CB | LYS | F | 379 | 50.992 | 37.369 | −22.950 | 1.00 | 111.55 | F |
| ATOM | 21068 | CG | LYS | F | 379 | 49.644 | 37.947 | −23.331 | 1.00 | 111.55 | F |
| ATOM | 21069 | CD | LYS | F | 379 | 48.982 | 37.092 | −24.401 | 1.00 | 111.55 | F |
| ATOM | 21070 | CE | LYS | F | 379 | 47.705 | 37.726 | −24.938 | 1.00 | 111.55 | F |
| ATOM | 21071 | NZ | LYS | F | 379 | 47.159 | 36.966 | −26.105 | 1.00 | 111.55 | F |
| ATOM | 21072 | C | LYS | F | 379 | 53.034 | 37.445 | −21.526 | 1.00 | 77.68 | F |
| ATOM | 21073 | O | LYS | F | 379 | 54.027 | 37.537 | −22.252 | 1.00 | 77.68 | F |
| ATOM | 21074 | N | TYR | F | 380 | 53.030 | 36.752 | −20.394 | 1.00 | 68.79 | F |
| ATOM | 21075 | CA | TYR | F | 380 | 54.209 | 36.012 | −19.951 | 1.00 | 68.79 | F |
| ATOM | 21076 | CB | TYR | F | 380 | 54.227 | 35.928 | −18.427 | 1.00 | 68.58 | F |
| ATOM | 21077 | CG | TYR | F | 380 | 55.460 | 35.281 | −17.862 | 1.00 | 68.58 | F |
| ATOM | 21078 | CD1 | TYR | F | 380 | 56.616 | 36.023 | −17.637 | 1.00 | 68.58 | F |
| ATOM | 21079 | CE1 | TYR | F | 380 | 57.760 | 35.424 | −17.106 | 1.00 | 68.58 | F |
| ATOM | 21080 | CD2 | TYR | F | 380 | 55.471 | 33.920 | −17.548 | 1.00 | 68.58 | F |
| ATOM | 21081 | CE2 | TYR | F | 380 | 56.605 | 33.310 | −17.021 | 1.00 | 68.58 | F |
| ATOM | 21082 | CZ | TYR | F | 380 | 57.748 | 34.067 | −16.800 | 1.00 | 68.58 | F |
| ATOM | 21083 | OH | TYR | F | 380 | 58.876 | 33.484 | −16.267 | 1.00 | 68.58 | F |
| ATOM | 21084 | C | TYR | F | 380 | 54.103 | 34.617 | −20.543 | 1.00 | 68.79 | F |
| ATOM | 21085 | O | TYR | F | 380 | 53.222 | 33.850 | −20.158 | 1.00 | 68.79 | F |
| ATOM | 21086 | N | VAL | F | 381 | 54.982 | 34.294 | −21.487 | 1.00 | 50.22 | F |
| ATOM | 21087 | CA | VAL | F | 381 | 54.953 | 32.977 | −22.126 | 1.00 | 50.22 | F |
| ATOM | 21088 | CB | VAL | F | 381 | 55.613 | 33.020 | −23.521 | 1.00 | 57.03 | F |
| ATOM | 21089 | CG1 | VAL | F | 381 | 55.448 | 31.682 | −24.218 | 1.00 | 57.03 | F |
| ATOM | 21090 | CG2 | VAL | F | 381 | 54.982 | 34.111 | −24.348 | 1.00 | 57.03 | F |
| ATOM | 21091 | C | VAL | F | 381 | 55.658 | 31.924 | −21.256 | 1.00 | 50.22 | F |
| ATOM | 21092 | O | VAL | F | 381 | 56.856 | 32.038 | −20.967 | 1.00 | 50.22 | F |
| ATOM | 21093 | N | ASN | F | 382 | 54.908 | 30.892 | −20.869 | 1.00 | 76.52 | F |
| ATOM | 21094 | CA | ASN | F | 382 | 55.421 | 29.846 | −20.000 | 1.00 | 76.52 | F |
| ATOM | 21095 | CB | ASN | F | 382 | 54.574 | 28.586 | −20.108 | 1.00 | 54.96 | F |
| ATOM | 21096 | CG | ASN | F | 382 | 54.668 | 27.717 | −18.554 | 1.00 | 54.96 | F |
| ATOM | 21097 | OD1 | ASN | F | 382 | 55.701 | 27.692 | −18.166 | 1.00 | 54.96 | F |
| ATOM | 21098 | ND2 | ASN | F | 382 | 53.592 | 26.990 | −18.560 | 1.00 | 54.96 | F |
| ATOM | 21099 | C | ASN | F | 382 | 56.880 | 29.462 | −20.180 | 1.00 | 76.52 | F |
| ATOM | 21100 | O | ASN | F | 382 | 57.744 | 29.899 | −19.421 | 1.00 | 76.52 | F |
| ATOM | 21101 | N | GLY | F | 383 | 57.180 | 28.647 | −21.173 | 1.00 | 42.41 | F |
| ATOM | 21102 | CA | GLY | F | 383 | 58.569 | 28.244 | −21.314 | 1.00 | 42.41 | F |
| ATOM | 21103 | C | GLY | F | 383 | 59.582 | 29.267 | −21.810 | 1.00 | 42.41 | F |
| ATOM | 21104 | O | GLY | F | 383 | 60.752 | 28.942 | −21.997 | 1.00 | 42.41 | F |
| ATOM | 21105 | N | ARG | F | 384 | 59.161 | 30.505 | −22.022 | 1.00 | 66.07 | F |
| ATOM | 21106 | CA | ARG | F | 384 | 60.086 | 31.501 | −22.536 | 1.00 | 66.07 | F |
| ATOM | 21107 | CB | ARG | F | 384 | 59.303 | 32.672 | −23.129 | 1.00 | 67.93 | F |
| ATOM | 21108 | CG | ARG | F | 384 | 60.163 | 33.689 | −23.871 | 1.00 | 67.93 | F |
| ATOM | 21109 | CD | ARG | F | 384 | 59.296 | 34.579 | −24.742 | 1.00 | 67.93 | F |
| ATOM | 21110 | NE | ARG | F | 384 | 58.553 | 33.780 | −25.712 | 1.00 | 67.93 | F |
| ATOM | 21111 | CZ | ARG | F | 384 | 57.561 | 34.238 | −26.472 | 1.00 | 67.93 | F |
| ATOM | 21112 | NH1 | ARG | F | 384 | 57.180 | 35.503 | −26.383 | 1.00 | 67.93 | F |
| ATOM | 21113 | NH2 | ARG | F | 384 | 56.938 | 33.426 | −27.319 | 1.00 | 67.93 | F |
| ATOM | 21114 | C | ARG | F | 384 | 61.074 | 32.005 | −21.496 | 1.00 | 66.07 | F |
| ATOM | 21115 | O | ARG | F | 384 | 60.774 | 32.043 | −20.307 | 1.00 | 66.07 | F |
| ATOM | 21116 | N | TYR | F | 385 | 62.260 | 32.382 | −21.953 | 1.00 | 70.43 | F |
| ATOM | 21117 | CA | TYR | F | 385 | 63.288 | 32.901 | −21.065 | 1.00 | 70.43 | F |
| ATOM | 21118 | CB | TYR | F | 385 | 64.675 | 32.622 | −21.644 | 1.00 | 104.79 | F |
| ATOM | 21119 | CG | TYR | F | 385 | 65.767 | 33.321 | −20.879 | 1.00 | 104.79 | F |
| ATOM | 21120 | CD1 | TYR | F | 385 | 66.179 | 32.850 | −19.636 | 1.00 | 104.79 | F |
| ATOM | 21121 | CE1 | TYR | F | 385 | 67.121 | 33.539 | −18.882 | 1.00 | 104.79 | F |
| ATOM | 21122 | CD2 | TYR | F | 385 | 66.331 | 34.502 | −21.357 | 1.00 | 104.79 | F |
| ATOM | 21123 | CE2 | TYR | F | 385 | 67.273 | 35.200 | −20.610 | 1.00 | 104.79 | F |
| ATOM | 21124 | CZ | TYR | F | 385 | 67.662 | 34.714 | −19.374 | 1.00 | 104.79 | F |
| ATOM | 21125 | OH | TYR | F | 385 | 68.577 | 35.415 | −18.622 | 1.00 | 104.79 | F |
| ATOM | 21126 | C | TYR | F | 385 | 63.126 | 34.414 | −20.880 | 1.00 | 70.43 | F |
| ATOM | 21127 | O | TYR | F | 385 | 62.798 | 35.131 | −21.830 | 1.00 | 70.43 | F |
| ATOM | 21128 | N | TYR | F | 386 | 63.362 | 34.903 | −19.663 | 1.00 | 76.85 | F |
| ATOM | 21129 | CA | TYR | F | 386 | 63.253 | 36.338 | −19.381 | 1.00 | 76.85 | F |
| ATOM | 21130 | CB | TYR | F | 386 | 61.881 | 36.712 | −18.800 | 1.00 | 79.81 | F |
| ATOM | 21131 | CG | TYR | F | 386 | 60.675 | 36.494 | −19.680 | 1.00 | 79.81 | F |
| ATOM | 21132 | CD1 | TYR | F | 386 | 59.912 | 35.337 | −19.566 | 1.00 | 79.81 | F |
| ATOM | 21133 | CE1 | TYR | F | 386 | 58.771 | 35.147 | −20.337 | 1.00 | 79.81 | F |
| ATOM | 21134 | CD2 | TYR | F | 386 | 60.271 | 37.463 | −20.598 | 1.00 | 79.81 | F |
| ATOM | 21135 | CE2 | TYR | F | 386 | 59.128 | 37.281 | −21.378 | 1.00 | 79.81 | F |
| ATOM | 21136 | CZ | TYR | F | 386 | 58.381 | 36.118 | −21.239 | 1.00 | 79.81 | F |
| ATOM | 21137 | OH | TYR | F | 386 | 57.241 | 35.928 | −21.991 | 1.00 | 79.81 | F |
| ATOM | 21138 | C | TYR | F | 386 | 64.273 | 36.815 | −18.359 | 1.00 | 76.85 | F |
| ATOM | 21139 | O | TYR | F | 386 | 64.338 | 36.277 | −17.254 | 1.00 | 76.85 | F |
| ATOM | 21140 | N | PRO | F | 387 | 65.096 | 37.817 | −18.708 | 1.00 | 107.09 | F |
| ATOM | 21141 | CD | PRO | F | 387 | 65.344 | 38.395 | −20.038 | 1.00 | 66.88 | F |
| ATOM | 21142 | CA | PRO | F | 387 | 66.073 | 38.312 | −17.729 | 1.00 | 107.09 | F |
| ATOM | 21143 | CB | PRO | F | 387 | 66.848 | 39.356 | −18.523 | 1.00 | 66.88 | F |
| ATOM | 21144 | CG | PRO | F | 387 | 66.783 | 38.828 | −19.924 | 1.00 | 66.88 | F |
| ATOM | 21145 | C | PRO | F | 387 | 65.146 | 38.936 | −16.684 | 1.00 | 107.09 | F |
| ATOM | 21146 | O | PRO | F | 387 | 64.099 | 39.465 | −17.062 | 1.00 | 107.09 | F |
| ATOM | 21147 | N | GLU | F | 388 | 65.480 | 38.915 | −15.396 | 1.00 | 131.10 | F |
| ATOM | 21148 | CA | GLU | F | 388 | 64.488 | 39.456 | −14.472 | 1.00 | 131.10 | F |
| ATOM | 21149 | CB | GLU | F | 388 | 63.648 | 38.300 | −13.929 | 1.00 | 130.96 | F |
| ATOM | 21150 | CG | GLU | F | 388 | 64.391 | 37.376 | −12.987 | 1.00 | 130.96 | F |
| ATOM | 21151 | CD | GLU | F | 388 | 63.455 | 36.427 | −12.268 | 1.00 | 130.96 | F |
| ATOM | 21152 | OE1 | GLU | F | 388 | 62.532 | 36.913 | −11.580 | 1.00 | 130.96 | F |
| ATOM | 21153 | OE2 | GLU | F | 388 | 63.642 | 35.199 | −12.392 | 1.00 | 130.96 | F |
| ATOM | 21154 | C | GLU | F | 388 | 64.747 | 40.409 | −13.306 | 1.00 | 131.10 | F |
| ATOM | 21155 | O | GLU | F | 388 | 63.905 | 40.483 | −12.412 | 1.00 | 131.10 | F |
| ATOM | 21156 | N | TRP | F | 389 | 65.851 | 41.144 | −13.274 | 1.00 | 100.63 | F |
| ATOM | 21157 | CA | TRP | F | 389 | 66.038 | 42.076 | −12.153 | 1.00 | 100.63 | F |
| ATOM | 21158 | CB | TRP | F | 389 | 65.006 | 43.208 | −12.236 | 1.00 | 110.57 | F |
| ATOM | 21159 | CG | TRP | F | 389 | 65.472 | 44.474 | −12.874 | 1.00 | 110.57 | F |
| ATOM | 21160 | CD2 | TRP | F | 389 | 66.813 | 44.812 | −13.258 | 1.00 | 110.57 | F |
| ATOM | 21161 | CE2 | TRP | F | 389 | 66.776 | 46.125 | −13.777 | 1.00 | 110.57 | F |
| ATOM | 21162 | CE3 | TRP | F | 389 | 68.042 | 44.136 | −13.211 | 1.00 | 110.57 | F |
| ATOM | 21163 | CD1 | TRP | F | 389 | 64.704 | 45.561 | −13.170 | 1.00 | 110.57 | F |
| ATOM | 21164 | NE1 | TRP | F | 389 | 65.477 | 46.556 | −13.712 | 1.00 | 110.57 | F |
| ATOM | 21165 | CZ2 | TRP | F | 389 | 67.922 | 46.778 | −14.248 | 1.00 | 110.57 | F |
| ATOM | 21166 | CZ3 | TRP | F | 389 | 69.183 | 44.785 | −13.679 | 1.00 | 110.57 | F |
| ATOM | 21167 | CH2 | TRP | F | 389 | 69.113 | 46.094 | −14.190 | 1.00 | 110.57 | F |
| ATOM | 21168 | C | TRP | F | 389 | 65.864 | 41.392 | −10.799 | 1.00 | 100.63 | F |
| ATOM | 21169 | O | TRP | F | 389 | 64.870 | 41.629 | −10.109 | 1.00 | 100.63 | F |
| ATOM | 21170 | N | GLY | F | 390 | 66.824 | 40.562 | −10.411 | 1.00 | 116.80 | F |
| ATOM | 21171 | CA | GLY | F | 390 | 66.713 | 39.868 | −9.140 | 1.00 | 116.80 | F |
| ATOM | 21172 | C | GLY | F | 390 | 66.706 | 40.771 | −7.921 | 1.00 | 116.80 | F |
| ATOM | 21173 | O | GLY | F | 390 | 67.234 | 41.879 | −7.958 | 1.00 | 116.80 | F |
| ATOM | 21174 | N | ARG | F | 391 | 66.094 | 40.294 | −6.841 | 1.00 | 115.28 | F |
| ATOM | 21175 | CA | ARG | F | 391 | 66.034 | 41.036 | −5.588 | 1.00 | 115.28 | F |
| ATOM | 21176 | CB | ARG | F | 391 | 64.736 | 40.698 | −4.842 | 1.00 | 111.27 | F |
| ATOM | 21177 | CG | ARG | F | 391 | 63.457 | 41.013 | −5.622 | 1.00 | 111.27 | F |
| ATOM | 21178 | CD | ARG | F | 391 | 62.850 | 42.382 | −5.271 | 1.00 | 111.27 | F |
| ATOM | 21179 | NE | ARG | F | 391 | 61.874 | 42.320 | −4.176 | 1.00 | 111.27 | F |
| ATOM | 21180 | CZ | ARG | F | 391 | 62.180 | 42.222 | −2.881 | 1.00 | 111.27 | F |
| ATOM | 21181 | NH1 | ARG | F | 391 | 63.448 | 42.180 | −2.489 | 1.00 | 111.27 | F |
| ATOM | 21182 | NH2 | ARG | F | 391 | 61.212 | 42.159 | −1.972 | 1.00 | 111.27 | F |
| ATOM | 21183 | C | ARG | F | 391 | 67.256 | 40.596 | −4.771 | 1.00 | 115.28 | F |
| ATOM | 21184 | O | ARG | F | 391 | 67.916 | 39.618 | −5.121 | 1.00 | 115.28 | F |
| ATOM | 21185 | N | LYS | F | 392 | 67.572 | 41.312 | −3.697 | 1.00 | 97.75 | F |
| ATOM | 21186 | CA | LYS | F | 392 | 68.724 | 40.952 | −2.873 | 1.00 | 97.75 | F |
| ATOM | 21187 | CB | LYS | F | 392 | 69.654 | 42.156 | −2.695 | 1.00 | 121.82 | F |
| ATOM | 21188 | CG | LYS | F | 392 | 70.879 | 41.851 | −1.854 | 1.00 | 121.82 | F |
| ATOM | 21189 | CD | LYS | F | 392 | 71.641 | 43.110 | −1.494 | 1.00 | 121.82 | F |
| ATOM | 21190 | CE | LYS | F | 392 | 72.766 | 42.806 | −0.510 | 1.00 | 121.82 | F |
| ATOM | 21191 | NZ | LYS | F | 392 | 73.423 | 44.048 | −0.003 | 1.00 | 121.82 | F |
| ATOM | 21192 | C | LYS | F | 392 | 68.291 | 40.437 | −1.501 | 1.00 | 97.75 | F |
| ATOM | 21193 | O | LYS | F | 392 | 67.724 | 41.181 | −0.700 | 1.00 | 97.75 | F |
| ATOM | 21194 | N | SER | F | 393 | 68.569 | 39.163 | −1.234 | 1.00 | 101.21 | F |
| ATOM | 21195 | CA | SER | F | 393 | 68.209 | 38.536 | 0.038 | 1.00 | 101.21 | F |
| ATOM | 21196 | CB | SER | F | 393 | 66.708 | 38.221 | 0.070 | 1.00 | 64.15 | F |
| ATOM | 21197 | OG | SER | F | 393 | 66.342 | 37.527 | 1.253 | 1.00 | 64.15 | F |
| ATOM | 21198 | C | SER | F | 393 | 68.994 | 37.244 | 0.206 | 1.00 | 101.21 | F |
| ATOM | 21199 | O | SER | F | 393 | 69.481 | 36.682 | −0.773 | 1.00 | 101.21 | F |
| ATOM | 21200 | N | ASN | F | 394 | 69.126 | 36.776 | 1.443 | 1.00 | 107.11 | F |
| ATOM | 21201 | CA | ASN | F | 394 | 69.842 | 35.530 | 1.688 | 1.00 | 107.11 | F |
| ATOM | 21202 | CB | ASN | F | 394 | 70.246 | 35.414 | 3.151 | 1.00 | 97.91 | F |
| ATOM | 21203 | CG | ASN | F | 394 | 71.356 | 36.364 | 3.515 | 1.00 | 97.91 | F |
| ATOM | 21204 | OD1 | ASN | F | 394 | 72.380 | 36.429 | 2.831 | 1.00 | 97.91 | F |
| ATOM | 21205 | ND2 | ASN | F | 394 | 71.166 | 37.106 | 4.602 | 1.00 | 97.91 | F |
| ATOM | 21206 | C | ASN | F | 394 | 68.945 | 34.368 | 1.311 | 1.00 | 107.11 | F |
| ATOM | 21207 | O | ASN | F | 394 | 69.410 | 33.250 | 1.100 | 1.00 | 107.11 | F |
| ATOM | 21208 | N | TYR | F | 395 | 67.650 | 34.651 | 1.233 | 1.00 | 64.27 | F |
| ATOM | 21209 | CA | TYR | F | 395 | 66.667 | 33.656 | 0.855 | 1.00 | 64.27 | F |
| ATOM | 21210 | CB | TYR | F | 395 | 65.384 | 33.858 | 1.656 | 1.00 | 80.82 | F |
| ATOM | 21211 | CG | TYR | F | 395 | 65.554 | 33.665 | 3.137 | 1.00 | 80.82 | F |
| ATOM | 21212 | CD1 | TYR | F | 395 | 65.815 | 34.745 | 3.976 | 1.00 | 80.82 | F |
| ATOM | 21213 | CE1 | TYR | F | 395 | 65.973 | 34.566 | 5.355 | 1.00 | 80.82 | F |
| ATOM | 21214 | CD2 | TYR | F | 395 | 65.456 | 32.397 | 3.705 | 1.00 | 80.82 | F |
| ATOM | 21215 | CE2 | TYR | F | 395 | 65.612 | 32.205 | 5.076 | 1.00 | 80.82 | F |
| ATOM | 21216 | CZ | TYR | F | 395 | 65.871 | 33.296 | 5.898 | 1.00 | 80.82 | F |
| ATOM | 21217 | OH | TYR | F | 395 | 66.029 | 33.109 | 7.256 | 1.00 | 80.82 | F |
| ATOM | 21218 | C | TYR | F | 395 | 66.349 | 33.782 | −0.633 | 1.00 | 64.27 | F |
| ATOM | 21219 | O | TYR | F | 395 | 66.518 | 34.845 | −1.227 | 1.00 | 64.27 | F |
| ATOM | 21220 | N | GLU | F | 396 | 65.890 | 32.689 | −1.234 | 1.00 | 84.94 | F |
| ATOM | 21221 | CA | GLU | F | 396 | 65.512 | 32.680 | −2.645 | 1.00 | 84.94 | F |
| ATOM | 21222 | CB | GLU | F | 396 | 65.998 | 31.402 | −3.325 | 1.00 | 150.21 | F |
| ATOM | 21223 | CG | GLU | F | 396 | 67.445 | 31.056 | −3.062 | 1.00 | 150.21 | F |
| ATOM | 21224 | CD | GLU | F | 396 | 67.789 | 29.658 | −3.539 | 1.00 | 150.21 | F |
| ATOM | 21225 | OE1 | GLU | F | 396 | 67.085 | 28.705 | −3.135 | 1.00 | 150.21 | F |
| ATOM | 21226 | OE2 | GLU | F | 396 | 68.761 | 29.509 | −4.312 | 1.00 | 150.21 | F |
| ATOM | 21227 | C | GLU | F | 396 | 63.986 | 32.702 | −2.672 | 1.00 | 84.94 | F |
| ATOM | 21228 | O | GLU | F | 396 | 63.338 | 32.030 | −1.863 | 1.00 | 84.94 | F |
| ATOM | 21229 | N | PRO | F | 397 | 63.385 | 33.474 | −3.593 | 1.00 | 76.85 | F |
| ATOM | 21230 | CD | PRO | F | 397 | 63.955 | 34.244 | −4.712 | 1.00 | 76.14 | F |
| ATOM | 21231 | CA | PRO | F | 397 | 61.918 | 33.501 | −3.630 | 1.00 | 76.85 | F |
| ATOM | 21232 | CB | PRO | F | 397 | 61.610 | 34.322 | −4.884 | 1.00 | 76.14 | F |
| ATOM | 21233 | CG | PRO | F | 397 | 62.851 | 34.160 | −5.731 | 1.00 | 76.14 | F |
| ATOM | 21234 | C | PRO | F | 397 | 61.369 | 32.081 | −3.680 | 1.00 | 76.85 | F |
| ATOM | 21235 | O | PRO | F | 397 | 61.846 | 31.241 | −4.437 | 1.00 | 76.85 | F |
| ATOM | 21236 | N | GLY | F | 398 | 60.370 | 31.822 | −2.851 | 1.00 | 47.27 | F |
| ATOM | 21237 | CA | GLY | F | 398 | 59.787 | 30.497 | −2.775 | 1.00 | 47.27 | F |
| ATOM | 21238 | C | GLY | F | 398 | 59.984 | 29.982 | −1.357 | 1.00 | 47.27 | F |
| ATOM | 21239 | O | GLY | F | 398 | 59.087 | 29.349 | −0.788 | 1.00 | 47.27 | F |
| ATOM | 21240 | N | GLU | F | 399 | 61.163 | 30.248 | −0.788 | 1.00 | 85.15 | F |
| ATOM | 21241 | CA | GLU | F | 399 | 61.471 | 29.829 | 0.580 | 1.00 | 85.15 | F |
| ATOM | 21242 | CB | GLU | F | 399 | 62.969 | 30.004 | 0.887 | 1.00 | 76.85 | F |
| ATOM | 21243 | CG | GLU | F | 399 | 63.897 | 29.317 | −0.102 | 1.00 | 76.85 | F |
| ATOM | 21244 | CD | GLU | F | 399 | 65.376 | 29.451 | 0.247 | 1.00 | 76.85 | F |
| ATOM | 21245 | OE1 | GLU | F | 399 | 65.804 | 30.563 | 0.602 | 1.00 | 76.85 | F |
| ATOM | 21246 | OE2 | GLU | F | 399 | 66.119 | 28.449 | 0.147 | 1.00 | 76.85 | F |
| ATOM | 21247 | C | GLU | F | 399 | 60.648 | 30.737 | 1.491 | 1.00 | 85.15 | F |
| ATOM | 21248 | O | GLU | F | 399 | 60.553 | 31.941 | 1.245 | 1.00 | 85.15 | F |
| ATOM | 21249 | N | PRO | F | 400 | 60.021 | 30.178 | 2.542 | 1.00 | 78.46 | F |
| ATOM | 21250 | CD | PRO | F | 400 | 59.909 | 28.778 | 2.978 | 1.00 | 57.39 | F |
| ATOM | 21251 | CA | PRO | F | 400 | 59.233 | 31.059 | 3.410 | 1.00 | 73.54 | F |
| ATOM | 21252 | CB | PRO | F | 400 | 58.676 | 30.107 | 4.473 | 1.00 | 106.00 | F |
| ATOM | 21253 | CG | PRO | F | 400 | 59.596 | 28.944 | 4.437 | 1.00 | 57.39 | F |
| ATOM | 21254 | C | PRO | F | 400 | 60.019 | 32.233 | 3.997 | 1.00 | 64.29 | F |
| ATOM | 21255 | O | PRO | F | 400 | 59.436 | 33.231 | 4.413 | 1.00 | 64.50 | F |
| ATOM | 21256 | N | GLY | F | 401 | 61.343 | 32.131 | 3.996 | 1.00 | 73.45 | F |
| ATOM | 21257 | CA | GLY | F | 401 | 62.157 | 33.209 | 4.528 | 1.00 | 73.01 | F |
| ATOM | 21258 | C | GLY | F | 401 | 62.273 | 34.441 | 3.639 | 1.00 | 72.80 | F |
| ATOM | 21259 | O | GLY | F | 401 | 62.801 | 35.465 | 4.066 | 1.00 | 73.51 | F |
| ATOM | 21260 | N | PHE | F | 402 | 61.789 | 34.364 | 2.405 | 1.00 | 104.17 | F |
| ATOM | 21261 | CA | PHE | F | 402 | 61.879 | 35.511 | 1.512 | 1.00 | 104.17 | F |
| ATOM | 21262 | CB | PHE | F | 402 | 61.517 | 35.116 | 0.086 | 1.00 | 174.24 | F |
| ATOM | 21263 | CG | PHE | F | 402 | 62.057 | 36.059 | −0.939 | 1.00 | 72.57 | F |
| ATOM | 21264 | CD1 | PHE | F | 402 | 63.410 | 36.034 | −1.275 | 1.00 | 72.57 | F |
| ATOM | 21265 | CD2 | PHE | F | 402 | 61.233 | 37.012 | −1.528 | 1.00 | 72.57 | F |
| ATOM | 21266 | CE1 | PHE | F | 402 | 63.937 | 36.949 | −2.185 | 1.00 | 72.57 | F |
| ATOM | 21267 | CE2 | PHE | F | 402 | 61.749 | 37.936 | −2.439 | 1.00 | 72.57 | F |
| ATOM | 21268 | CZ | PHE | F | 402 | 63.106 | 37.904 | −2.769 | 1.00 | 72.57 | F |
| ATOM | 21269 | C | PHE | F | 402 | 60.962 | 36.642 | 1.977 | 1.00 | 104.17 | F |
| ATOM | 21270 | O | PHE | F | 402 | 59.778 | 36.425 | 2.237 | 1.00 | 104.17 | F |
| ATOM | 21271 | N | PRO | F | 403 | 61.503 | 37.870 | 2.075 | 1.00 | 106.90 | F |
| ATOM | 21272 | CD | PRO | F | 403 | 62.908 | 38.200 | 1.791 | 1.00 | 86.17 | F |
| ATOM | 21273 | CA | PRO | F | 403 | 60.783 | 39.071 | 2.506 | 1.00 | 106.90 | F |
| ATOM | 21274 | CB | PRO | F | 403 | 61.893 | 40.114 | 2.677 | 1.00 | 191.00 | F |
| ATOM | 21275 | CG | PRO | F | 403 | 63.151 | 39.323 | 2.748 | 1.00 | 86.17 | F |
| ATOM | 21276 | C | PRO | F | 403 | 59.738 | 39.566 | 1.515 | 1.00 | 106.90 | F |
| ATOM | 21277 | O | PRO | F | 403 | 60.077 | 40.194 | 0.512 | 1.00 | 106.90 | F |
| ATOM | 21278 | N | TRP | F | 404 | 58.472 | 39.277 | 1.785 | 1.00 | 117.41 | F |
| ATOM | 21279 | CA | TRP | F | 404 | 57.393 | 39.755 | 0.930 | 1.00 | 117.41 | F |
| ATOM | 21280 | CB | TRP | F | 404 | 56.551 | 38.602 | 0.375 | 1.00 | 105.51 | F |
| ATOM | 21281 | CG | TRP | F | 404 | 57.192 | 37.820 | −0.737 | 1.00 | 98.75 | F |
| ATOM | 21282 | CD2 | TRP | F | 404 | 57.599 | 38.311 | −2.025 | 1.00 | 98.75 | F |
| ATOM | 21283 | CE2 | TRP | F | 404 | 58.109 | 37.212 | −2.746 | 1.00 | 98.75 | F |
| ATOM | 21284 | CE3 | TRP | F | 404 | 57.582 | 39.571 | −2.638 | 1.00 | 98.75 | F |
| ATOM | 21285 | CD1 | TRP | F | 404 | 57.467 | 36.484 | −0.735 | 1.00 | 98.75 | F |
| ATOM | 21286 | NE1 | TRP | F | 404 | 58.016 | 36.112 | −1.935 | 1.00 | 98.75 | F |
| ATOM | 21287 | CZ2 | TRP | F | 404 | 58.598 | 37.332 | −4.054 | 1.00 | 98.75 | F |
| ATOM | 21288 | CZ3 | TRP | F | 404 | 58.070 | 39.689 | −3.943 | 1.00 | 98.75 | F |
| ATOM | 21289 | CH2 | TRP | F | 404 | 58.570 | 38.576 | −4.632 | 1.00 | 98.75 | F |
| ATOM | 21290 | C | TRP | F | 404 | 56.529 | 40.607 | 1.841 | 1.00 | 117.41 | F |
| ATOM | 21291 | O | TRP | F | 404 | 56.168 | 40.170 | 2.935 | 1.00 | 117.41 | F |
| ATOM | 21292 | N | ASN | F | 405 | 56.208 | 41.821 | 1.406 | 1.00 | 73.58 | F |
| ATOM | 21293 | CA | ASN | F | 405 | 55.378 | 42.702 | 2.219 | 1.00 | 73.61 | F |
| ATOM | 21294 | CB | ASN | F | 405 | 55.550 | 44.152 | 1.767 | 1.00 | 93.30 | F |
| ATOM | 21295 | CG | ASN | F | 405 | 55.189 | 45.139 | 2.851 | 1.00 | 93.30 | F |
| ATOM | 21296 | OD1 | ASN | F | 405 | 54.018 | 45.303 | 3.194 | 1.00 | 93.30 | F |
| ATOM | 21297 | ND2 | ASN | F | 405 | 56.201 | 45.794 | 3.412 | 1.00 | 93.30 | F |
| ATOM | 21298 | C | ASN | F | 405 | 53.910 | 42.284 | 2.130 | 1.00 | 82.55 | F |
| ATOM | 21299 | O | ASN | F | 405 | 53.096 | 42.926 | 1.460 | 1.00 | 75.61 | F |
| ATOM | 21300 | N | ILE | F | 406 | 53.585 | 41.190 | 2.807 | 1.00 | 95.34 | F |
| ATOM | 21301 | CA | ILE | F | 406 | 52.228 | 40.670 | 2.815 | 1.00 | 87.54 | F |
| ATOM | 21302 | CB | ILE | F | 406 | 52.195 | 39.165 | 2.455 | 1.00 | 88.80 | F |
| ATOM | 21303 | CG2 | ILE | F | 406 | 50.750 | 38.676 | 2.353 | 1.00 | 76.42 | F |
| ATOM | 21304 | CG1 | ILE | F | 406 | 52.924 | 38.935 | 1.130 | 1.00 | 76.42 | F |
| ATOM | 21305 | CD1 | ILE | F | 406 | 52.903 | 37.495 | 0.652 | 1.00 | 76.42 | F |
| ATOM | 21306 | C | ILE | F | 406 | 51.628 | 40.839 | 4.198 | 1.00 | 94.41 | F |
| ATOM | 21307 | O | ILE | F | 406 | 52.311 | 40.679 | 5.210 | 1.00 | 89.51 | F |
| ATOM | 21308 | N | LYS | F | 407 | 50.348 | 41.179 | 4.238 | 1.00 | 91.19 | F |
| ATOM | 21309 | CA | LYS | F | 407 | 49.636 | 41.346 | 5.495 | 1.00 | 86.79 | F |
| ATOM | 21310 | CB | LYS | F | 407 | 49.506 | 42.822 | 5.871 | 1.00 | 107.53 | F |
| ATOM | 21311 | CG | LYS | F | 407 | 50.816 | 43.540 | 6.117 | 1.00 | 93.63 | F |
| ATOM | 21312 | CD | LYS | F | 407 | 50.562 | 44.928 | 6.695 | 1.00 | 93.63 | F |
| ATOM | 21313 | CE | LYS | F | 407 | 51.843 | 45.739 | 6.822 | 1.00 | 93.63 | F |
| ATOM | 21314 | NZ | LYS | F | 407 | 52.437 | 46.048 | 5.487 | 1.00 | 93.63 | F |
| ATOM | 21315 | C | LYS | F | 407 | 48.260 | 40.776 | 5.249 | 1.00 | 96.10 | F |
| ATOM | 21316 | O | LYS | F | 407 | 47.700 | 40.950 | 4.166 | 1.00 | 95.04 | F |
| ATOM | 21317 | N | LYS | F | 408 | 47.719 | 40.070 | 6.231 | 1.00 | 100.37 | F |
| ATOM | 21318 | CA | LYS | F | 408 | 46.386 | 39.521 | 6.067 | 1.00 | 99.96 | F |
| ATOM | 21319 | CB | LYS | F | 408 | 46.093 | 38.483 | 7.145 | 1.00 | 71.89 | F |
| ATOM | 21320 | CG | LYS | F | 408 | 44.769 | 37.781 | 6.946 | 1.00 | 70.69 | F |
| ATOM | 21321 | CD | LYS | F | 408 | 44.653 | 36.575 | 7.853 | 1.00 | 70.69 | F |
| ATOM | 21322 | CE | LYS | F | 408 | 43.369 | 35.810 | 7.578 | 1.00 | 70.69 | F |
| ATOM | 21323 | NZ | LYS | F | 408 | 43.316 | 34.552 | 8.371 | 1.00 | 70.69 | F |
| ATOM | 21324 | C | LYS | F | 408 | 45.464 | 40.715 | 6.226 | 1.00 | 100.09 | F |
| ATOM | 21325 | O | LYS | F | 408 | 45.311 | 41.233 | 7.328 | 1.00 | 98.66 | F |
| ATOM | 21326 | N | ASP | F | 409 | 44.857 | 41.173 | 5.137 | 1.00 | 146.26 | F |
| ATOM | 21327 | CA | ASP | F | 409 | 43.997 | 42.340 | 5.250 | 1.00 | 146.44 | F |
| ATOM | 21328 | CB | ASP | F | 409 | 43.546 | 42.840 | 3.862 | 1.00 | 127.44 | F |
| ATOM | 21329 | CG | ASP | F | 409 | 43.053 | 41.729 | 2.954 | 1.00 | 120.26 | F |
| ATOM | 21330 | OD1 | ASP | F | 409 | 42.241 | 40.898 | 3.412 | 1.00 | 120.26 | F |
| ATOM | 21331 | OD2 | ASP | F | 409 | 43.468 | 41.700 | 1.772 | 1.00 | 120.26 | F |
| ATOM | 21332 | C | ASP | F | 409 | 42.798 | 42.145 | 6.178 | 1.00 | 146.35 | F |
| ATOM | 21333 | O | ASP | F | 409 | 42.441 | 41.015 | 6.530 | 1.00 | 150.08 | F |
| ATOM | 21334 | N | LYS | F | 410 | 42.222 | 43.278 | 6.586 | 1.00 | 128.14 | F |
| ATOM | 21335 | CA | LYS | F | 410 | 41.067 | 43.366 | 7.482 | 1.00 | 134.58 | F |
| ATOM | 21336 | CB | LYS | F | 410 | 40.192 | 44.562 | 7.088 | 1.00 | 175.88 | F |
| ATOM | 21337 | CG | LYS | F | 410 | 39.471 | 44.420 | 5.741 | 1.00 | 115.33 | F |
| ATOM | 21338 | CD | LYS | F | 410 | 40.421 | 44.527 | 4.543 | 1.00 | 115.33 | F |
| ATOM | 21339 | CE | LYS | F | 410 | 39.683 | 44.337 | 3.213 | 1.00 | 115.33 | F |
| ATOM | 21340 | NZ | LYS | F | 410 | 40.591 | 44.427 | 2.028 | 1.00 | 115.33 | F |
| ATOM | 21341 | C | LYS | F | 410 | 40.195 | 42.120 | 7.546 | 1.00 | 133.40 | F |
| ATOM | 21342 | O | LYS | F | 410 | 40.145 | 41.336 | 6.600 | 1.00 | 132.93 | F |
| ATOM | 21343 | N | ASP | F | 411 | 39.495 | 41.962 | 8.666 | 1.00 | 86.87 | F |
| ATOM | 21344 | CA | ASP | F | 411 | 38.611 | 40.819 | 8.894 | 1.00 | 84.38 | F |
| ATOM | 21345 | CB | ASP | F | 411 | 37.361 | 41.253 | 9.666 | 1.00 | 183.80 | F |
| ATOM | 21346 | CG | ASP | F | 411 | 37.557 | 41.200 | 11.168 | 1.00 | 183.80 | F |
| ATOM | 21347 | OD1 | ASP | F | 411 | 37.996 | 40.144 | 11.672 | 1.00 | 183.80 | F |
| ATOM | 21348 | OD2 | ASP | F | 411 | 37.266 | 42.206 | 11.847 | 1.00 | 183.80 | F |
| ATOM | 21349 | C | ASP | F | 411 | 38.179 | 40.060 | 7.643 | 1.00 | 85.99 | F |
| ATOM | 21350 | O | ASP | F | 411 | 37.091 | 40.284 | 7.107 | 1.00 | 90.17 | F |
| ATOM | 21351 | N | ALA | F | 412 | 39.043 | 39.150 | 7.197 | 1.00 | 178.26 | F |
| ATOM | 21352 | CA | ALA | F | 412 | 38.790 | 38.330 | 6.016 | 1.00 | 191.28 | F |
| ATOM | 21353 | CB | ALA | F | 412 | 38.813 | 39.190 | 4.759 | 1.00 | 60.70 | F |
| ATOM | 21354 | C | ALA | F | 412 | 39.846 | 37.236 | 5.913 | 1.00 | 185.98 | F |
| ATOM | 21355 | O | ALA | F | 412 | 40.822 | 37.227 | 6.662 | 1.00 | 181.03 | F |
| ATOM | 21356 | N | ASN | F | 413 | 39.645 | 36.316 | 4.977 | 1.00 | 124.87 | F |
| ATOM | 21357 | CA | ASN | F | 413 | 40.579 | 35.218 | 4.775 | 1.00 | 117.68 | F |
| ATOM | 21358 | CB | ASN | F | 413 | 39.839 | 33.986 | 4.266 | 1.00 | 93.50 | F |
| ATOM | 21359 | CG | ASN | F | 413 | 38.798 | 33.496 | 5.238 | 1.00 | 93.50 | F |
| ATOM | 21360 | OD1 | ASN | F | 413 | 39.035 | 33.459 | 6.447 | 1.00 | 93.50 | F |
| ATOM | 21361 | ND2 | ASN | F | 413 | 37.640 | 33.102 | 4.720 | 1.00 | 93.50 | F |
| ATOM | 21362 | C | ASN | F | 413 | 41.651 | 35.581 | 3.769 | 1.00 | 122.26 | F |
| ATOM | 21363 | O | ASN | F | 413 | 42.674 | 34.909 | 3.663 | 1.00 | 121.95 | F |
| ATOM | 21364 | N | THR | F | 414 | 41.408 | 36.656 | 3.034 | 1.00 | 72.74 | F |
| ATOM | 21365 | CA | THR | F | 414 | 42.312 | 37.111 | 1.998 | 1.00 | 75.78 | F |
| ATOM | 21366 | CB | THR | F | 414 | 41.517 | 37.960 | 0.999 | 1.00 | 129.14 | F |
| ATOM | 21367 | OG1 | THR | F | 414 | 42.390 | 38.415 | −0.029 | 1.00 | 99.63 | F |
| ATOM | 21368 | CG2 | THR | F | 414 | 40.867 | 39.147 | 1.690 | 1.00 | 99.63 | F |
| ATOM | 21369 | C | THR | F | 414 | 43.558 | 37.867 | 2.494 | 1.00 | 75.24 | F |
| ATOM | 21370 | O | THR | F | 414 | 43.631 | 38.258 | 3.659 | 1.00 | 76.31 | F |
| ATOM | 21371 | N | TYR | F | 415 | 44.541 | 38.046 | 1.608 | 1.00 | 61.97 | F |
| ATOM | 21372 | CA | TYR | F | 415 | 45.796 | 38.742 | 1.932 | 1.00 | 63.00 | F |
| ATOM | 21373 | CB | TYR | F | 415 | 46.980 | 37.765 | 1.966 | 1.00 | 93.98 | F |
| ATOM | 21374 | CG | TYR | F | 415 | 46.928 | 36.741 | 3.070 | 1.00 | 60.92 | F |
| ATOM | 21375 | CD1 | TYR | F | 415 | 45.966 | 35.729 | 3.067 | 1.00 | 60.92 | F |
| ATOM | 21376 | CE1 | TYR | F | 415 | 45.905 | 34.792 | 4.089 | 1.00 | 60.92 | F |
| ATOM | 21377 | CD2 | TYR | F | 415 | 47.835 | 36.789 | 4.133 | 1.00 | 60.92 | F |
| ATOM | 21378 | CE2 | TYR | F | 415 | 47.782 | 35.856 | 5.167 | 1.00 | 60.92 | F |
| ATOM | 21379 | CZ | TYR | F | 415 | 46.816 | 34.862 | 5.136 | 1.00 | 60.92 | F |
| ATOM | 21380 | OH | TYR | F | 415 | 46.760 | 33.934 | 6.152 | 1.00 | 60.92 | F |
| ATOM | 21381 | C | TYR | F | 415 | 46.119 | 39.816 | 0.900 | 1.00 | 70.56 | F |
| ATOM | 21382 | O | TYR | F | 415 | 45.575 | 39.814 | −0.202 | 1.00 | 65.21 | F |
| ATOM | 21383 | N | SER | F | 416 | 47.028 | 40.719 | 1.254 | 1.00 | 73.16 | F |
| ATOM | 21384 | CA | SER | F | 416 | 47.435 | 41.792 | 0.352 | 1.00 | 71.11 | F |
| ATOM | 21385 | CB | SER | F | 416 | 46.781 | 43.116 | 0.754 | 1.00 | 104.94 | F |
| ATOM | 21386 | OG | SER | F | 416 | 45.407 | 43.127 | 0.409 | 1.00 | 81.32 | F |
| ATOM | 21387 | C | SER | F | 416 | 48.945 | 41.941 | 0.359 | 1.00 | 77.30 | F |
| ATOM | 21388 | O | SER | F | 416 | 49.611 | 41.647 | 1.356 | 1.00 | 75.67 | F |
| ATOM | 21389 | N | LEU | F | 417 | 49.477 | 42.422 | −0.756 | 1.00 | 57.99 | F |
| ATOM | 21390 | CA | LEU | F | 417 | 50.916 | 42.577 | −0.910 | 1.00 | 56.89 | F |
| ATOM | 21391 | CB | LEU | F | 417 | 51.456 | 41.415 | −1.752 | 1.00 | 130.10 | F |
| ATOM | 21392 | CG | LEU | F | 417 | 52.733 | 41.699 | −2.548 | 1.00 | 88.21 | F |
| ATOM | 21393 | CD1 | LEU | F | 417 | 53.937 | 41.629 | −1.629 | 1.00 | 88.21 | F |
| ATOM | 21394 | CD2 | LEU | F | 417 | 52.864 | 40.705 | −3.680 | 1.00 | 88.21 | F |
| ATOM | 21395 | C | LEU | F | 417 | 51.318 | 43.885 | −1.566 | 1.00 | 68.40 | F |
| ATOM | 21396 | O | LEU | F | 417 | 50.572 | 44.446 | −2.373 | 1.00 | 63.50 | F |
| ATOM | 21397 | N | THR | F | 418 | 52.516 | 44.351 | −1.228 | 1.00 | 83.04 | F |
| ATOM | 21398 | CA | THR | F | 418 | 53.058 | 45.578 | −1.792 | 1.00 | 76.10 | F |
| ATOM | 21399 | CB | THR | F | 418 | 52.938 | 46.754 | −0.804 | 1.00 | 121.32 | F |
| ATOM | 21400 | OG1 | THR | F | 418 | 51.566 | 47.148 | −0.692 | 1.00 | 89.73 | F |
| ATOM | 21401 | CG2 | THR | F | 418 | 53.759 | 47.941 | −1.280 | 1.00 | 89.73 | F |
| ATOM | 21402 | C | THR | F | 418 | 54.526 | 45.369 | −2.103 | 1.00 | 91.29 | F |
| ATOM | 21403 | O | THR | F | 418 | 55.321 | 45.176 | −1.191 | 1.00 | 84.76 | F |
| ATOM | 21404 | N | ASP | F | 419 | 54.890 | 45.402 | −3.381 | 1.00 | 72.58 | F |
| ATOM | 21405 | CA | ASP | F | 419 | 56.288 | 45.213 | −3.752 | 1.00 | 77.69 | F |
| ATOM | 21406 | CB | ASP | F | 419 | 56.443 | 44.004 | −4.680 | 1.00 | 159.91 | F |
| ATOM | 21407 | CG | ASP | F | 419 | 57.888 | 43.547 | −4.802 | 1.00 | 137.50 | F |
| ATOM | 21408 | OD1 | ASP | F | 419 | 58.547 | 43.376 | −3.755 | 1.00 | 137.50 | F |
| ATOM | 21409 | OD2 | ASP | F | 419 | 58.368 | 43.352 | −5.936 | 1.00 | 137.50 | F |
| ATOM | 21410 | C | ASP | F | 419 | 56.851 | 46.467 | −4.416 | 1.00 | 81.78 | F |
| ATOM | 21411 | O | ASP | F | 419 | 56.308 | 46.953 | −5.409 | 1.00 | 76.67 | F |
| ATOM | 21412 | N | LEU | F | 420 | 57.947 | 46.980 | −3.861 | 1.00 | 75.85 | F |
| ATOM | 21413 | CA | LEU | F | 420 | 58.580 | 48.195 | −4.362 | 1.00 | 80.54 | F |
| ATOM | 21414 | CB | LEU | F | 420 | 58.886 | 49.124 | −3.183 | 1.00 | 156.01 | F |
| ATOM | 21415 | CG | LEU | F | 420 | 59.092 | 50.609 | −3.479 | 1.00 | 114.96 | F |
| ATOM | 21416 | CD1 | LEU | F | 420 | 57.787 | 51.200 | −3.997 | 1.00 | 114.96 | F |
| ATOM | 21417 | CD2 | LEU | F | 420 | 59.543 | 51.334 | −2.216 | 1.00 | 114.96 | F |
| ATOM | 21418 | C | LEU | F | 420 | 59.864 | 47.897 | −5.133 | 1.00 | 83.11 | F |
| ATOM | 21419 | O | LEU | F | 420 | 60.509 | 46.876 | −4.901 | 1.00 | 82.43 | F |
| ATOM | 21420 | N | VAL | F | 421 | 60.234 | 48.790 | −6.050 | 1.00 | 126.73 | F |
| ATOM | 21421 | CA | VAL | F | 421 | 61.447 | 48.615 | −6.848 | 1.00 | 125.24 | F |
| ATOM | 21422 | CB | VAL | F | 421 | 61.137 | 47.921 | −8.188 | 1.00 | 136.82 | F |
| ATOM | 21423 | CG1 | VAL | F | 421 | 62.376 | 47.883 | −9.065 | 1.00 | 92.25 | F |
| ATOM | 21424 | CG2 | VAL | F | 421 | 60.667 | 46.522 | −7.930 | 1.00 | 92.25 | F |
| ATOM | 21425 | C | VAL | F | 421 | 62.152 | 49.935 | −7.141 | 1.00 | 127.50 | F |
| ATOM | 21426 | O | VAL | F | 421 | 61.514 | 50.923 | −7.499 | 1.00 | 123.10 | F |
| ATOM | 21427 | N | ARG | F | 422 | 63.474 | 49.933 | −6.992 | 1.00 | 105.21 | F |
| ATOM | 21428 | CA | ARG | F | 422 | 64.297 | 51.113 | −7.236 | 1.00 | 107.34 | F |
| ATOM | 21429 | CB | ARG | F | 422 | 65.611 | 50.978 | −6.465 | 1.00 | 181.93 | F |
| ATOM | 21430 | CG | ARG | F | 422 | 65.932 | 52.127 | −5.528 | 1.00 | 142.52 | F |
| ATOM | 21431 | CD | ARG | F | 422 | 66.472 | 53.337 | −6.272 | 1.00 | 142.52 | F |
| ATOM | 21432 | NE | ARG | F | 422 | 66.917 | 54.376 | −5.346 | 1.00 | 142.52 | F |
| ATOM | 21433 | CZ | ARG | F | 422 | 67.559 | 55.484 | −5.706 | 1.00 | 142.52 | F |
| ATOM | 21434 | NH1 | ARG | F | 422 | 67.838 | 55.710 | −6.984 | 1.00 | 142.52 | F |
| ATOM | 21435 | NH2 | ARG | F | 422 | 67.927 | 56.366 | −4.786 | 1.00 | 142.52 | F |
| ATOM | 21436 | C | ARG | F | 422 | 64.590 | 51.243 | −8.730 | 1.00 | 104.57 | F |
| ATOM | 21437 | O | ARG | F | 422 | 65.470 | 50.559 | −9.250 | 1.00 | 95.24 | F |
| ATOM | 21438 | N | THR | F | 423 | 63.861 | 52.120 | −9.417 | 1.00 | 132.46 | F |
| ATOM | 21439 | CA | THR | F | 423 | 64.050 | 52.313 | −10.855 | 1.00 | 142.11 | F |
| ATOM | 21440 | CB | THR | F | 423 | 62.895 | 53.147 | −11.480 | 1.00 | 195.79 | F |
| ATOM | 21441 | OG1 | THR | F | 423 | 61.640 | 52.519 | −11.199 | 1.00 | 151.58 | F |
| ATOM | 21442 | CG2 | THR | F | 423 | 63.056 | 53.248 | −12.996 | 1.00 | 151.58 | F |
| ATOM | 21443 | C | THR | F | 423 | 65.365 | 53.014 | −11.178 | 1.00 | 140.33 | F |
| ATOM | 21444 | O | THR | F | 423 | 65.363 | 54.120 | −11.712 | 1.00 | 138.82 | F |
| ATOM | 21445 | N | THR | F | 424 | 66.488 | 52.379 | −10.855 | 1.00 | 175.73 | F |
| ATOM | 21446 | CA | THR | F | 424 | 67.788 | 52.975 | −11.143 | 1.00 | 175.73 | F |
| ATOM | 21447 | CB | THR | F | 424 | 68.941 | 52.112 | −10.594 | 1.00 | 199.50 | F |
| ATOM | 21448 | OG1 | THR | F | 424 | 68.768 | 51.931 | −9.184 | 1.00 | 145.31 | F |
| ATOM | 21449 | CG2 | THR | F | 424 | 70.286 | 52.789 | −10.846 | 1.00 | 145.31 | F |
| ATOM | 21450 | C | THR | F | 424 | 67.938 | 53.114 | −12.655 | 1.00 | 175.73 | F |
| ATOM | 21451 | O | THR | F | 424 | 67.298 | 52.391 | −13.419 | 1.00 | 175.73 | F |
| ATOM | 21452 | N | SER | F | 425 | 68.783 | 54.045 | −13.085 | 1.00 | 180.14 | F |
| ATOM | 21453 | CA | SER | F | 425 | 68.991 | 54.285 | −14.507 | 1.00 | 180.14 | F |
| ATOM | 21454 | CB | SER | F | 425 | 69.680 | 55.634 | −14.707 | 1.00 | 200.84 | F |
| ATOM | 21455 | OG | SER | F | 425 | 69.794 | 55.947 | −16.083 | 1.00 | 104.56 | F |
| ATOM | 21456 | C | SER | F | 425 | 69.792 | 53.191 | −15.213 | 1.00 | 180.14 | F |
| ATOM | 21457 | O | SER | F | 425 | 70.018 | 53.266 | −16.420 | 1.00 | 180.14 | F |
| ATOM | 21458 | N | LYS | F | 426 | 70.218 | 52.177 | −14.465 | 1.00 | 163.16 | F |
| ATOM | 21459 | CA | LYS | F | 426 | 70.991 | 51.076 | −15.038 | 1.00 | 163.16 | F |
| ATOM | 21460 | CB | LYS | F | 426 | 71.305 | 50.039 | −13.961 | 1.00 | 200.84 | F |
| ATOM | 21461 | CG | LYS | F | 426 | 71.933 | 50.610 | −12.712 | 1.00 | 147.29 | F |
| ATOM | 21462 | CD | LYS | F | 426 | 72.033 | 49.545 | −11.640 | 1.00 | 147.29 | F |
| ATOM | 21463 | CE | LYS | F | 426 | 72.523 | 50.129 | −10.330 | 1.00 | 147.29 | F |
| ATOM | 21464 | NZ | LYS | F | 426 | 72.583 | 49.087 | −9.268 | 1.00 | 147.29 | F |
| ATOM | 21465 | C | LYS | F | 426 | 70.228 | 50.391 | −16.171 | 1.00 | 163.16 | F |
| ATOM | 21466 | O | LYS | F | 426 | 70.811 | 50.010 | −17.187 | 1.00 | 163.16 | F |
| ATOM | 21467 | N | MET | F | 427 | 68.920 | 50.236 | −15.982 | 1.00 | 200.84 | F |
| ATOM | 21468 | CA | MET | F | 427 | 68.056 | 49.587 | −16.966 | 1.00 | 200.84 | F |
| ATOM | 21469 | CB | MET | F | 427 | 66.660 | 49.373 | −16.374 | 1.00 | 159.14 | F |
| ATOM | 21470 | CG | MET | F | 427 | 66.035 | 50.625 | −15.786 | 1.00 | 159.14 | F |
| ATOM | 21471 | SD | MET | F | 427 | 64.422 | 50.290 | −15.063 | 1.00 | 159.14 | F |
| ATOM | 21472 | CE | MET | F | 427 | 64.898 | 49.536 | −13.496 | 1.00 | 159.14 | F |
| ATOM | 21473 | C | MET | F | 427 | 67.938 | 50.359 | −18.274 | 1.00 | 200.84 | F |
| ATOM | 21474 | O | MET | F | 427 | 68.766 | 51.218 | −18.578 | 1.00 | 200.84 | F |
| ATOM | 21475 | N | SER | F | 428 | 66.901 | 50.044 | −19.046 | 1.00 | 125.20 | F |
| ATOM | 21476 | CA | SER | F | 428 | 66.682 | 50.704 | −20.325 | 1.00 | 126.85 | F |
| ATOM | 21477 | CB | SER | F | 428 | 67.786 | 50.310 | −21.310 | 1.00 | 125.30 | F |
| ATOM | 21478 | OG | SER | F | 428 | 67.758 | 48.915 | −21.570 | 1.00 | 122.01 | F |
| ATOM | 21479 | C | SER | F | 428 | 65.323 | 50.374 | −20.937 | 1.00 | 122.80 | F |
| ATOM | 21480 | O | SER | F | 428 | 65.187 | 50.343 | −22.159 | 1.00 | 122.68 | F |
| ATOM | 21481 | N | SER | F | 429 | 64.319 | 50.131 | −20.100 | 1.00 | 140.53 | F |
| ATOM | 21482 | CA | SER | F | 429 | 62.985 | 49.809 | −20.604 | 1.00 | 139.20 | F |
| ATOM | 21483 | CB | SER | F | 429 | 62.930 | 48.352 | −21.084 | 1.00 | 141.71 | F |
| ATOM | 21484 | OG | SER | F | 429 | 63.802 | 48.123 | −22.176 | 1.00 | 141.71 | F |
| ATOM | 21485 | C | SER | F | 429 | 61.891 | 50.017 | −19.565 | 1.00 | 137.58 | F |
| ATOM | 21486 | O | SER | F | 429 | 60.707 | 49.955 | −19.889 | 1.00 | 126.28 | F |
| ATOM | 21487 | N | GLN | F | 430 | 62.287 | 50.269 | −18.323 | 1.00 | 148.86 | F |
| ATOM | 21488 | CA | GLN | F | 430 | 61.329 | 50.445 | −17.235 | 1.00 | 148.86 | F |
| ATOM | 21489 | CB | GLN | F | 430 | 60.301 | 51.521 | −17.558 | 1.00 | 180.66 | F |
| ATOM | 21490 | CG | GLN | F | 430 | 59.340 | 51.741 | −16.416 | 1.00 | 109.65 | F |
| ATOM | 21491 | CD | GLN | F | 430 | 58.289 | 52.766 | −16.734 | 1.00 | 109.65 | F |
| ATOM | 21492 | OE1 | GLN | F | 430 | 57.413 | 52.536 | −17.568 | 1.00 | 109.65 | F |
| ATOM | 21493 | NE2 | GLN | F | 430 | 58.370 | 53.915 | −16.075 | 1.00 | 109.65 | F |
| ATOM | 21494 | C | GLN | F | 430 | 60.615 | 49.120 | −17.024 | 1.00 | 148.86 | F |
| ATOM | 21495 | O | GLN | F | 430 | 59.826 | 48.683 | −17.863 | 1.00 | 148.86 | F |
| ATOM | 21496 | N | PRO | F | 431 | 60.863 | 48.473 | −15.883 | 1.00 | 99.95 | F |
| ATOM | 21497 | CD | PRO | F | 431 | 61.501 | 48.994 | −14.662 | 1.00 | 107.89 | F |
| ATOM | 21498 | CA | PRO | F | 431 | 60.225 | 47.191 | −15.616 | 1.00 | 99.94 | F |
| ATOM | 21499 | CB | PRO | F | 431 | 60.799 | 46.804 | −14.259 | 1.00 | 200.84 | F |
| ATOM | 21500 | CG | PRO | F | 431 | 60.899 | 48.118 | −13.582 | 1.00 | 177.41 | F |
| ATOM | 21501 | C | PRO | F | 431 | 58.711 | 47.230 | −15.612 | 1.00 | 96.56 | F |
| ATOM | 21502 | O | PRO | F | 431 | 58.091 | 48.280 | −15.440 | 1.00 | 96.44 | F |
| ATOM | 21503 | N | VAL | F | 432 | 58.139 | 46.054 | −15.828 | 1.00 | 76.51 | F |
| ATOM | 21504 | CA | VAL | F | 432 | 56.708 | 45.842 | −15.823 | 1.00 | 78.89 | F |
| ATOM | 21505 | CB | VAL | F | 432 | 56.236 | 45.289 | −17.173 | 1.00 | 116.29 | F |
| ATOM | 21506 | CG1 | VAL | F | 432 | 56.914 | 43.969 | −17.472 | 1.00 | 88.37 | F |
| ATOM | 21507 | CG2 | VAL | F | 432 | 54.758 | 45.114 | −17.151 | 1.00 | 88.37 | F |
| ATOM | 21508 | C | VAL | F | 432 | 56.545 | 44.794 | −14.724 | 1.00 | 76.89 | F |
| ATOM | 21509 | O | VAL | F | 432 | 57.447 | 43.981 | −14.499 | 1.00 | 67.25 | F |
| ATOM | 21510 | N | CYS | F | 433 | 55.421 | 44.806 | −14.027 | 1.00 | 68.03 | F |
| ATOM | 21511 | CA | CYS | F | 433 | 55.242 | 43.852 | −12.946 | 1.00 | 63.91 | F |
| ATOM | 21512 | C | CYS | F | 433 | 54.518 | 42.582 | −13.383 | 1.00 | 61.55 | F |
| ATOM | 21513 | O | CYS | F | 433 | 53.408 | 42.648 | −13.919 | 1.00 | 62.29 | F |
| ATOM | 21514 | CB | CYS | F | 433 | 54.484 | 44.514 | −11.788 | 1.00 | 162.83 | F |
| ATOM | 21515 | SG | CYS | F | 433 | 54.322 | 43.486 | −10.291 | 1.00 | 109.94 | F |
| ATOM | 21516 | N | VAL | F | 434 | 55.154 | 41.427 | −13.162 | 1.00 | 57.62 | F |
| ATOM | 21517 | CA | VAL | F | 434 | 54.553 | 40.138 | −13.498 | 1.00 | 53.61 | F |
| ATOM | 21518 | CB | VAL | F | 434 | 55.529 | 39.223 | −14.265 | 1.00 | 62.10 | F |
| ATOM | 21519 | CG1 | VAL | F | 434 | 54.792 | 37.975 | −14.743 | 1.00 | 43.88 | F |
| ATOM | 21520 | CG2 | VAL | F | 434 | 56.135 | 39.969 | −15.442 | 1.00 | 43.88 | F |
| ATOM | 21521 | C | VAL | F | 434 | 54.175 | 39.464 | −12.187 | 1.00 | 64.01 | F |
| ATOM | 21522 | O | VAL | F | 434 | 55.035 | 39.180 | −11.358 | 1.00 | 55.07 | F |
| ATOM | 21523 | N | VAL | F | 435 | 52.884 | 39.211 | −12.016 | 1.00 | 69.76 | F |
| ATOM | 21524 | CA | VAL | F | 435 | 52.364 | 38.604 | −10.797 | 1.00 | 61.50 | F |
| ATOM | 21525 | CB | VAL | F | 435 | 51.106 | 39.359 | −10.319 | 1.00 | 81.43 | F |
| ATOM | 21526 | CG1 | VAL | F | 435 | 50.418 | 38.599 | −9.199 | 1.00 | 60.76 | F |
| ATOM | 21527 | CG2 | VAL | F | 435 | 51.498 | 40.748 | −9.863 | 1.00 | 60.76 | F |
| ATOM | 21528 | C | VAL | F | 435 | 52.006 | 37.138 | −10.935 | 1.00 | 69.42 | F |
| ATOM | 21529 | O | VAL | F | 435 | 51.172 | 36.785 | −11.768 | 1.00 | 70.01 | F |
| ATOM | 21530 | N | PHE | F | 436 | 52.624 | 36.294 | −10.106 | 1.00 | 47.95 | F |
| ATOM | 21531 | CA | PHE | F | 436 | 52.352 | 34.861 | −10.128 | 1.00 | 44.15 | F |
| ATOM | 21532 | CB | PHE | F | 436 | 53.652 | 34.070 | −10.087 | 1.00 | 87.69 | F |
| ATOM | 21533 | CG | PHE | F | 436 | 54.526 | 34.289 | −11.280 | 1.00 | 62.46 | F |
| ATOM | 21534 | CD1 | PHE | F | 436 | 55.440 | 35.335 | −11.308 | 1.00 | 62.46 | F |
| ATOM | 21535 | CD2 | PHE | F | 436 | 54.435 | 33.444 | −12.382 | 1.00 | 62.46 | F |
| ATOM | 21536 | CE1 | PHE | F | 436 | 56.261 | 35.533 | −12.423 | 1.00 | 62.46 | F |
| ATOM | 21537 | CE2 | PHE | F | 436 | 55.248 | 33.632 | −13.502 | 1.00 | 62.46 | F |
| ATOM | 21538 | CZ | PHE | F | 436 | 56.164 | 34.677 | −13.524 | 1.00 | 62.46 | F |
| ATOM | 21539 | C | PHE | F | 436 | 51.478 | 34.472 | −8.950 | 1.00 | 51.98 | F |
| ATOM | 21540 | O | PHE | F | 436 | 51.848 | 34.669 | −7.794 | 1.00 | 51.36 | F |
| ATOM | 21541 | N | HIS | F | 437 | 50.310 | 33.924 | −9.253 | 1.00 | 46.08 | F |
| ATOM | 21542 | CA | HIS | F | 437 | 49.373 | 33.511 | −8.221 | 1.00 | 47.15 | F |
| ATOM | 21543 | CB | HIS | F | 437 | 48.509 | 34.699 | −7.797 | 1.00 | 97.62 | F |
| ATOM | 21544 | CG | HIS | F | 437 | 47.527 | 34.372 | −6.717 | 1.00 | 73.99 | F |
| ATOM | 21545 | CD2 | HIS | F | 437 | 46.212 | 34.053 | −6.774 | 1.00 | 73.99 | F |
| ATOM | 21546 | ND1 | HIS | F | 437 | 47.884 | 34.292 | −5.388 | 1.00 | 73.99 | F |
| ATOM | 21547 | CE1 | HIS | F | 437 | 46.830 | 33.937 | −4.674 | 1.00 | 73.99 | F |
| ATOM | 21548 | NE2 | HIS | F | 437 | 45.803 | 33.785 | −5.491 | 1.00 | 73.99 | F |
| ATOM | 21549 | C | HIS | F | 437 | 48.484 | 32.395 | −8.764 | 1.00 | 51.46 | F |
| ATOM | 21550 | O | HIS | F | 437 | 48.359 | 32.242 | −9.976 | 1.00 | 53.68 | F |
| ATOM | 21551 | N | ASP | F | 438 | 47.868 | 31.616 | −7.876 | 1.00 | 55.12 | F |
| ATOM | 21552 | CA | ASP | F | 438 | 46.997 | 30.520 | −8.294 | 1.00 | 57.03 | F |
| ATOM | 21553 | CB | ASP | F | 438 | 46.395 | 29.831 | −7.069 | 1.00 | 107.74 | F |
| ATOM | 21554 | CG | ASP | F | 438 | 47.326 | 28.783 | −6.484 | 1.00 | 78.70 | F |
| ATOM | 21555 | OD1 | ASP | F | 438 | 48.554 | 28.876 | −6.728 | 1.00 | 78.70 | F |
| ATOM | 21556 | OD2 | ASP | F | 438 | 46.831 | 27.875 | −5.782 | 1.00 | 78.70 | F |
| ATOM | 21557 | C | ASP | F | 438 | 45.908 | 30.985 | −9.256 | 1.00 | 58.37 | F |
| ATOM | 21558 | O | ASP | F | 438 | 45.665 | 30.357 | −10.284 | 1.00 | 61.93 | F |
| ATOM | 21559 | N | THR | F | 439 | 45.238 | 32.077 | −8.922 | 1.00 | 91.75 | F |
| ATOM | 21560 | CA | THR | F | 439 | 44.238 | 32.628 | −9.818 | 1.00 | 89.10 | F |
| ATOM | 21561 | CB | THR | F | 439 | 43.315 | 33.578 | −9.061 | 1.00 | 79.77 | F |
| ATOM | 21562 | OG1 | THR | F | 439 | 44.081 | 34.669 | −8.544 | 1.00 | 65.58 | F |
| ATOM | 21563 | CG2 | THR | F | 439 | 42.666 | 32.850 | −7.891 | 1.00 | 65.58 | F |
| ATOM | 21564 | C | THR | F | 439 | 45.158 | 33.389 | −10.772 | 1.00 | 94.87 | F |
| ATOM | 21565 | O | THR | F | 439 | 46.194 | 33.887 | −10.346 | 1.00 | 95.21 | F |
| ATOM | 21566 | N | LEU | F | 440 | 44.813 | 33.477 | −12.049 | 1.00 | 100.40 | F |
| ATOM | 21567 | CA | LEU | F | 440 | 45.684 | 34.143 | −13.027 | 1.00 | 102.42 | F |
| ATOM | 21568 | CB | LEU | F | 440 | 45.713 | 35.686 | −12.840 | 1.00 | 92.16 | F |
| ATOM | 21569 | CG | LEU | F | 440 | 46.164 | 36.656 | −11.711 | 1.00 | 72.16 | F |
| ATOM | 21570 | CD1 | LEU | F | 440 | 45.210 | 36.541 | −10.553 | 1.00 | 72.16 | F |
| ATOM | 21571 | CD2 | LEU | F | 440 | 47.603 | 36.446 | −11.265 | 1.00 | 72.16 | F |
| ATOM | 21572 | C | LEU | F | 440 | 47.115 | 33.589 | −13.002 | 1.00 | 112.37 | F |
| ATOM | 21573 | O | LEU | F | 440 | 48.021 | 34.229 | −12.480 | 1.00 | 110.59 | F |
| ATOM | 21574 | N | GLU | F | 441 | 47.313 | 32.403 | −13.580 | 1.00 | 76.86 | F |
| ATOM | 21575 | CA | GLU | F | 441 | 48.629 | 31.748 | −13.620 | 1.00 | 76.86 | F |
| ATOM | 21576 | CB | GLU | F | 441 | 48.716 | 30.805 | −14.821 | 1.00 | 197.59 | F |
| ATOM | 21577 | CG | GLU | F | 441 | 47.760 | 29.627 | −14.745 | 1.00 | 120.95 | F |
| ATOM | 21578 | CD | GLU | F | 441 | 47.985 | 28.623 | −15.861 | 1.00 | 120.95 | F |
| ATOM | 21579 | OE1 | GLU | F | 441 | 47.914 | 29.020 | −17.046 | 1.00 | 120.95 | F |
| ATOM | 21580 | OE2 | GLU | F | 441 | 48.236 | 27.437 | −15.552 | 1.00 | 120.95 | F |
| ATOM | 21581 | C | GLU | F | 441 | 49.821 | 32.707 | −13.637 | 1.00 | 76.86 | F |
| ATOM | 21582 | O | GLU | F | 441 | 50.799 | 32.497 | −12.925 | 1.00 | 76.86 | F |
| ATOM | 21583 | N | ALA | F | 442 | 49.738 | 33.755 | −14.448 | 1.00 | 80.16 | F |
| ATOM | 21584 | CA | ALA | F | 442 | 50.809 | 34.738 | −14.517 | 1.00 | 78.38 | F |
| ATOM | 21585 | CB | ALA | F | 442 | 52.074 | 34.098 | −15.063 | 1.00 | 37.61 | F |
| ATOM | 21586 | C | ALA | F | 442 | 50.386 | 35.910 | −15.392 | 1.00 | 74.37 | F |
| ATOM | 21587 | O | ALA | F | 442 | 50.314 | 35.780 | −16.612 | 1.00 | 71.78 | F |
| ATOM | 21588 | N | GLN | F | 443 | 50.099 | 37.050 | −14.768 | 1.00 | 80.32 | F |
| ATOM | 21589 | CA | GLN | F | 443 | 49.681 | 38.241 | −15.500 | 1.00 | 83.60 | F |
| ATOM | 21590 | CB | GLN | F | 443 | 48.339 | 38.746 | −14.986 | 1.00 | 90.76 | F |
| ATOM | 21591 | CG | GLN | F | 443 | 47.149 | 38.161 | −15.704 | 1.00 | 73.99 | F |
| ATOM | 21592 | CD | GLN | F | 443 | 45.899 | 39.007 | −15.511 | 1.00 | 73.99 | F |
| ATOM | 21593 | OE1 | GLN | F | 443 | 45.920 | 40.231 | −15.723 | 1.00 | 73.99 | F |
| ATOM | 21594 | NE2 | GLN | F | 443 | 44.801 | 38.363 | −15.113 | 1.00 | 73.99 | F |
| ATOM | 21595 | C | GLN | F | 443 | 50.691 | 39.371 | −15.430 | 1.00 | 85.77 | F |
| ATOM | 21596 | O | GLN | F | 443 | 51.417 | 39.518 | −14.448 | 1.00 | 86.83 | F |
| ATOM | 21597 | N | VAL | F | 444 | 50.729 | 40.172 | −16.486 | 1.00 | 66.04 | F |
| ATOM | 21598 | CA | VAL | F | 444 | 51.646 | 41.297 | −16.554 | 1.00 | 63.19 | F |
| ATOM | 21599 | CB | VAL | F | 444 | 52.322 | 41.356 | −17.937 | 1.00 | 80.95 | F |
| ATOM | 21600 | CG1 | VAL | F | 444 | 53.546 | 42.247 | −17.877 | 1.00 | 73.25 | F |
| ATOM | 21601 | CG2 | VAL | F | 444 | 52.710 | 39.960 | −18.381 | 1.00 | 73.25 | F |
| ATOM | 21602 | C | VAL | F | 444 | 50.891 | 42.608 | −16.291 | 1.00 | 67.01 | F |
| ATOM | 21603 | O | VAL | F | 444 | 49.751 | 42.781 | −16.727 | 1.00 | 69.27 | F |
| ATOM | 21604 | N | TYR | F | 445 | 51.523 | 43.518 | −15.556 | 1.00 | 76.12 | F |
| ATOM | 21605 | CA | TYR | F | 445 | 50.922 | 44.809 | −15.238 | 1.00 | 75.19 | F |
| ATOM | 21606 | CB | TYR | F | 445 | 50.428 | 44.808 | −13.807 | 1.00 | 88.60 | F |
| ATOM | 21607 | CG | TYR | F | 445 | 49.414 | 43.730 | −13.552 | 1.00 | 61.02 | F |
| ATOM | 21608 | CD1 | TYR | F | 445 | 49.805 | 42.463 | −13.115 | 1.00 | 61.02 | F |
| ATOM | 21609 | CE1 | TYR | F | 445 | 48.858 | 41.461 | −12.864 | 1.00 | 61.02 | F |
| ATOM | 21610 | CD2 | TYR | F | 445 | 48.056 | 43.975 | −13.741 | 1.00 | 61.02 | F |
| ATOM | 21611 | CE2 | TYR | F | 445 | 47.101 | 42.987 | −13.498 | 1.00 | 61.02 | F |
| ATOM | 21612 | CZ | TYR | F | 445 | 47.503 | 41.734 | −13.058 | 1.00 | 61.02 | F |
| ATOM | 21613 | OH | TYR | F | 445 | 46.547 | 40.770 | −12.799 | 1.00 | 61.02 | F |
| ATOM | 21614 | C | TYR | F | 445 | 51.931 | 45.931 | −15.453 | 1.00 | 76.65 | F |
| ATOM | 21615 | O | TYR | F | 445 | 53.099 | 45.808 | −15.074 | 1.00 | 79.51 | F |
| ATOM | 21616 | N | THR | F | 446 | 51.473 | 47.034 | −16.040 | 1.00 | 114.99 | F |
| ATOM | 21617 | CA | THR | F | 446 | 52.358 | 48.153 | −16.361 | 1.00 | 121.97 | F |
| ATOM | 21618 | CB | THR | F | 446 | 52.349 | 48.402 | −17.880 | 1.00 | 89.06 | F |
| ATOM | 21619 | OG1 | THR | F | 446 | 51.005 | 48.663 | −18.302 | 1.00 | 77.21 | F |
| ATOM | 21620 | CG2 | THR | F | 446 | 52.863 | 47.194 | −18.631 | 1.00 | 77.21 | F |
| ATOM | 21621 | C | THR | F | 446 | 52.114 | 49.511 | −15.692 | 1.00 | 131.77 | F |
| ATOM | 21622 | O | THR | F | 446 | 51.519 | 49.613 | −14.617 | 1.00 | 135.44 | F |
| ATOM | 21623 | N | CYS | F | 447 | 52.624 | 50.534 | −16.384 | 1.00 | 138.87 | F |
| ATOM | 21624 | CA | CYS | F | 447 | 52.564 | 51.965 | −16.060 | 1.00 | 139.85 | F |
| ATOM | 21625 | C | CYS | F | 447 | 53.246 | 52.526 | −14.812 | 1.00 | 139.88 | F |
| ATOM | 21626 | O | CYS | F | 447 | 53.236 | 51.929 | −13.735 | 1.00 | 136.74 | F |
| ATOM | 21627 | CB | CYS | F | 447 | 51.113 | 52.475 | −16.122 | 1.00 | 183.64 | F |
| ATOM | 21628 | SG | CYS | F | 447 | 49.916 | 51.707 | −14.988 | 1.00 | 169.17 | F |
| ATOM | 21629 | N | SER | F | 448 | 53.845 | 53.700 | −15.001 | 1.00 | 200.84 | F |
| ATOM | 21630 | CA | SER | F | 448 | 54.551 | 54.424 | −13.951 | 1.00 | 200.84 | F |
| ATOM | 21631 | CB | SER | F | 448 | 56.052 | 54.461 | −14.246 | 1.00 | 200.84 | F |
| ATOM | 21632 | OG | SER | F | 448 | 56.587 | 53.152 | −14.332 | 1.00 | 200.84 | F |
| ATOM | 21633 | C | SER | F | 448 | 54.000 | 55.846 | −13.871 | 1.00 | 200.84 | F |
| ATOM | 21634 | O | SER | F | 448 | 52.855 | 56.089 | −14.260 | 1.00 | 200.84 | F |
| ATOM | 21635 | N | GLU | F | 449 | 54.817 | 56.784 | −13.392 | 1.00 | 188.03 | F |
| ATOM | 21636 | CA | GLU | F | 449 | 54.385 | 58.173 | −13.229 | 1.00 | 188.03 | F |
| ATOM | 21637 | CB | GLU | F | 449 | 53.741 | 58.710 | −14.516 | 1.00 | 130.46 | F |
| ATOM | 21638 | CG | GLU | F | 449 | 54.729 | 59.170 | −15.572 | 1.00 | 130.46 | F |
| ATOM | 21639 | CD | GLU | F | 449 | 55.842 | 58.173 | −15.790 | 1.00 | 130.46 | F |
| ATOM | 21640 | OE1 | GLU | F | 449 | 55.535 | 56.994 | −16.065 | 1.00 | 130.46 | F |
| ATOM | 21641 | OE2 | GLU | F | 449 | 57.023 | 58.569 | −15.683 | 1.00 | 130.46 | F |
| ATOM | 21642 | C | GLU | F | 449 | 53.353 | 58.167 | −12.110 | 1.00 | 188.03 | F |
| ATOM | 21643 | O | GLU | F | 449 | 53.357 | 59.021 | −11.227 | 1.00 | 188.03 | F |
| ATOM | 21644 | N | GLY | F | 450 | 52.481 | 57.169 | −12.164 | 1.00 | 78.05 | F |
| ATOM | 21645 | CA | GLY | F | 450 | 51.427 | 56.996 | −11.187 | 1.00 | 78.05 | F |
| ATOM | 21646 | C | GLY | F | 450 | 50.275 | 56.380 | −11.947 | 1.00 | 78.05 | F |
| ATOM | 21647 | O | GLY | F | 450 | 49.154 | 56.302 | −11.454 | 1.00 | 78.05 | F |
| ATOM | 21648 | N | CYS | F | 451 | 50.573 | 55.938 | −13.164 | 1.00 | 183.90 | F |
| ATOM | 21649 | CA | CYS | F | 451 | 49.577 | 55.342 | −14.039 | 1.00 | 183.90 | F |
| ATOM | 21650 | CB | CYS | F | 451 | 48.975 | 54.098 | −13.406 | 1.00 | 167.07 | F |
| ATOM | 21651 | SG | CYS | F | 451 | 50.165 | 52.744 | −13.266 | 1.00 | 167.07 | F |
| ATOM | 21652 | C | CYS | F | 451 | 48.480 | 56.348 | −14.290 | 1.00 | 183.90 | F |
| ATOM | 21653 | O | CYS | F | 451 | 47.311 | 56.013 | −14.006 | 1.00 | 183.90 | F |
| ATOM | 21654 | OXT | CYS | F | 451 | 48.813 | 57.454 | −14.763 | 1.00 | 139.44 | F |
| ATOM | 21655 | OH2 | TIP | A | 452 | −25.070 | −37.082 | 29.874 | 1.00 | 26.29 | A |
| ATOM | 21656 | OH2 | TIP | A | 453 | −12.398 | −7.529 | 28.666 | 1.00 | 50.13 | A |
| ATOM | 21657 | OH2 | TIP | A | 454 | −9.057 | −11.779 | 29.287 | 1.00 | 43.25 | A |
| ATOM | 21658 | OH2 | TIP | A | 455 | −5.254 | 11.199 | 15.730 | 1.00 | 51.68 | A |
| ATOM | 21659 | OH2 | TIP | A | 456 | −32.698 | −22.663 | 13.953 | 1.00 | 66.28 | A |
| ATOM | 21660 | OH2 | TIP | A | 457 | −18.641 | −27.224 | 16.840 | 1.00 | 56.61 | A |
| ATOM | 21661 | OH2 | TIP | A | 458 | −25.885 | −27.088 | 20.474 | 1.00 | 51.71 | A |
| ATOM | 21662 | OH2 | TIP | A | 459 | −3.550 | −9.452 | 11.111 | 1.00 | 37.25 | A |
| ATOM | 21663 | OH2 | TIP | A | 460 | 0.719 | −11.029 | 13.512 | 1.00 | 48.51 | A |
| ATOM | 21664 | OH2 | TIP | A | 461 | −19.884 | −5.459 | 18.116 | 1.00 | 52.88 | A |
| ATOM | 21665 | OH2 | TIP | A | 462 | −20.875 | −28.974 | 41.287 | 1.00 | 44.76 | A |
| ATOM | 21666 | OH2 | TIP | A | 463 | −6.264 | −10.599 | 28.617 | 1.00 | 49.54 | A |
| ATOM | 21667 | OH2 | TIP | A | 464 | −15.397 | −15.617 | 18.480 | 1.00 | 52.38 | A |
| ATOM | 21668 | OH2 | TIP | A | 465 | −11.604 | −32.604 | 17.813 | 1.00 | 55.15 | A |
| ATOM | 21669 | OH2 | TIP | A | 466 | −4.100 | −8.096 | 13.946 | 1.00 | 52.37 | A |
| ATOM | 21670 | OH2 | TIP | A | 467 | −20.351 | 8.083 | 12.958 | 1.00 | 35.74 | A |
| ATOM | 21671 | OH2 | TIP | B | 452 | 5.473 | 25.059 | 67.902 | 1.00 | 26.29 | B |
| ATOM | 21672 | OH2 | TIP | B | 453 | −3.879 | 10.049 | 40.726 | 1.00 | 50.13 | B |
| ATOM | 21673 | OH2 | TIP | B | 454 | 2.008 | 9.463 | 42.344 | 1.00 | 43.25 | B |
| ATOM | 21674 | OH2 | TIP | B | 455 | −14.450 | −8.279 | 30.517 | 1.00 | 51.68 | B |
| ATOM | 21675 | OH2 | TIP | B | 456 | −13.917 | 12.739 | 68.373 | 1.00 | 66.28 | B |
| ATOM | 21676 | OH2 | TIP | B | 457 | −0.165 | 8.079 | 64.205 | 1.00 | 56.61 | B |
| ATOM | 21677 | OH2 | TIP | B | 458 | −5.764 | 16.338 | 64.985 | 1.00 | 51.71 | B |
| ATOM | 21678 | OH2 | TIP | B | 459 | −2.589 | −8.867 | 47.353 | 1.00 | 37.25 | B |
| ATOM | 21679 | OH2 | TIP | B | 460 | 2.297 | −9.443 | 45.553 | 1.00 | 48.51 | B |
| ATOM | 21680 | OH2 | TIP | B | 461 | −14.112 | 5.654 | 47.666 | 1.00 | 52.88 | B |
| ATOM | 21681 | OH2 | TIP | B | 462 | 7.527 | 29.221 | 53.940 | 1.00 | 44.76 | B |
| ATOM | 21682 | OH2 | TIP | B | 463 | 2.171 | 7.038 | 40.480 | 1.00 | 49.54 | B |
| ATOM | 21683 | OH2 | TIP | B | 464 | −4.585 | 5.110 | 53.365 | 1.00 | 52.38 | B |
| ATOM | 21684 | OH2 | TIP | B | 465 | 8.911 | 6.284 | 64.949 | 1.00 | 55.15 | B |
| ATOM | 21685 | OH2 | TIP | B | 466 | −3.027 | −6.304 | 44.890 | 1.00 | 52.37 | B |
| ATOM | 21686 | OH2 | TIP | B | 467 | −24.001 | −1.506 | 39.948 | 1.00 | 35.74 | B |
| ATOM | 21687 | OH2 | TIP | C | 452 | 4.024 | 24.034 | −10.748 | 1.00 | 26.29 | C |
| ATOM | 21688 | OH2 | TIP | C | 453 | −3.754 | 10.576 | 18.128 | 1.00 | 50.13 | C |
| ATOM | 21689 | OH2 | TIP | C | 454 | 1.043 | 9.077 | 15.439 | 1.00 | 43.25 | C |
| ATOM | 21690 | OH2 | TIP | C | 455 | −3.568 | 13.031 | 40.887 | 1.00 | 51.68 | C |
| ATOM | 21691 | OH2 | TIP | C | 456 | −2.771 | 37.525 | 7.450 | 1.00 | 66.28 | C |
| ATOM | 21692 | OH2 | TIP | C | 457 | 9.221 | 27.781 | 6.164 | 1.00 | 56.61 | C |
| ATOM | 21693 | OH2 | TIP | C | 458 | 0.751 | 29.872 | 1.818 | 1.00 | 51.71 | C |
| ATOM | 21694 | OH2 | TIP | C | 459 | 10.622 | 18.769 | 28.680 | 1.00 | 37.25 | C |
| ATOM | 21695 | OH2 | TIP | C | 460 | 14.203 | 14.549 | 28.076 | 1.00 | 48.51 | C |
| ATOM | 21696 | OH2 | TIP | C | 461 | −5.772 | 22.658 | 22.124 | 1.00 | 52.88 | C |
| ATOM | 21697 | OH2 | TIP | C | 462 | −2.831 | 10.753 | −7.603 | 1.00 | 44.76 | C |
| ATOM | 21698 | OH2 | TIP | C | 463 | 2.860 | 7.524 | 17.778 | 1.00 | 49.54 | C |
| ATOM | 21699 | OH2 | TIP | C | 464 | 4.007 | 21.061 | 16.717 | 1.00 | 52.38 | C |
| ATOM | 21700 | OH2 | TIP | C | 465 | 15.934 | 23.391 | 3.911 | 1.00 | 55.15 | C |
| ATOM | 21701 | OH2 | TIP | C | 466 | 8.071 | 17.151 | 28.026 | 1.00 | 52.37 | C |
| ATOM | 21702 | OH2 | TIP | C | 467 | −11.439 | 24.219 | 35.327 | 1.00 | 35.74 | C |
| ATOM | 21703 | OH2 | TIP | D | 452 | 45.268 | 29.003 | −67.249 | 1.00 | 26.29 | D |
| ATOM | 21704 | OH2 | TIP | D | 453 | 56.797 | 13.362 | −40.946 | 1.00 | 50.13 | D |
| ATOM | 21705 | OH2 | TIP | D | 454 | 50.903 | 13.373 | −42.094 | 1.00 | 43.25 | D |
| ATOM | 21706 | OH2 | TIP | D | 455 | 67.726 | −5.016 | −32.013 | 1.00 | 51.68 | D |
| ATOM | 21707 | OH2 | TIP | D | 456 | 63.736 | 15.430 | −69.676 | 1.00 | 66.28 | D |
| ATOM | 21708 | OH2 | TIP | D | 457 | 50.223 | 11.768 | −64.145 | 1.00 | 56.61 | D |
| ATOM | 21709 | OH2 | TIP | D | 458 | 56.193 | 20.359 | −66.232 | 1.00 | 51.71 | D |
| ATOM | 21710 | OH2 | TIP | D | 459 | 54.232 | −5.287 | −47.334 | 1.00 | 37.25 | D |
| ATOM | 21711 | OH2 | TIP | D | 460 | 49.094 | −5.727 | −45.008 | 1.00 | 48.51 | D |
| ATOM | 21712 | OH2 | TIP | D | 461 | 66.287 | 8.885 | −49.223 | 1.00 | 52.88 | D |
| ATOM | 21713 | OH2 | TIP | D | 462 | 45.723 | 33.240 | −52.765 | 1.00 | 44.76 | D |
| ATOM | 21714 | OH2 | TIP | D | 463 | 50.808 | 10.801 | −40.404 | 1.00 | 49.54 | D |
| ATOM | 21715 | OH2 | TIP | D | 464 | 55.928 | 8.879 | −53.601 | 1.00 | 52.38 | D |
| ATOM | 21716 | OH2 | TIP | D | 465 | 41.439 | 10.938 | −63.575 | 1.00 | 55.15 | D |
| ATOM | 21717 | OH2 | TIP | D | 466 | 54.885 | −2.614 | −45.203 | 1.00 | 52.37 | D |
| ATOM | 21718 | OH2 | TIP | D | 467 | 76.382 | 1.303 | −42.824 | 1.00 | 35.74 | D |
| ATOM | 21719 | OH2 | TIP | E | 452 | 78.797 | −34.064 | −33.425 | 1.00 | 26.29 | E |
| ATOM | 21720 | OH2 | TIP | E | 453 | 65.806 | −4.337 | −29.986 | 1.00 | 50.13 | E |
| ATOM | 21721 | OH2 | TIP | E | 454 | 62.920 | −8.466 | −30.344 | 1.00 | 43.25 | E |
| ATOM | 21722 | OH2 | TIP | E | 455 | 61.459 | 14.258 | −16.519 | 1.00 | 51.68 | E |
| ATOM | 21723 | OH2 | TIP | E | 456 | 88.278 | −19.664 | −17.874 | 1.00 | 66.28 | E |
| ATOM | 21724 | OH2 | TIP | E | 457 | 73.931 | −24.037 | −19.137 | 1.00 | 56.61 | E |
| ATOM | 21725 | OH2 | TIP | E | 458 | 80.667 | −23.704 | −23.668 | 1.00 | 51.71 | E |
| ATOM | 21726 | OH2 | TIP | E | 459 | 59.111 | −6.011 | −11.737 | 1.00 | 37.25 | E |
| ATOM | 21727 | OH2 | TIP | E | 460 | 54.206 | −7.763 | −12.773 | 1.00 | 48.51 | E |
| ATOM | 21728 | OH2 | TIP | E | 461 | 74.734 | −2.013 | −20.732 | 1.00 | 52.88 | E |
| ATOM | 21729 | OH2 | TIP | E | 462 | 72.384 | −24.633 | −43.695 | 1.00 | 44.76 | E |
| ATOM | 21730 | OH2 | TIP | E | 463 | 60.287 | −7.010 | −29.381 | 1.00 | 49.54 | E |
| ATOM | 21731 | OH2 | TIP | E | 464 | 70.824 | −12.289 | −20.457 | 1.00 | 52.38 | E |
| ATOM | 21732 | OH2 | TIP | E | 465 | 67.057 | −29.755 | −19.644 | 1.00 | 55.15 | E |
| ATOM | 21733 | OH2 | TIP | E | 466 | 59.372 | −4.764 | −14.557 | 1.00 | 52.37 | E |
| ATOM | 21734 | OH2 | TIP | E | 467 | 75.818 | 10.774 | −14.880 | 1.00 | 35.74 | E |
| ATOM | 21735 | OH2 | TIP | F | 452 | 55.607 | 27.153 | 11.357 | 1.00 | 26.29 | F |
| ATOM | 21736 | OH2 | TIP | F | 453 | 59.034 | 13.968 | −18.301 | 1.00 | 50.13 | F |
| ATOM | 21737 | OH2 | TIP | F | 454 | 55.113 | 12.363 | −15.078 | 1.00 | 43.25 | F |
| ATOM | 21738 | OH2 | TIP | F | 455 | 57.188 | 16.070 | −41.291 | 1.00 | 51.68 | F |
| ATOM | 21739 | OH2 | TIP | F | 456 | 60.706 | 40.941 | −6.962 | 1.00 | 66.28 | F |
| ATOM | 21740 | OH2 | TIP | F | 457 | 48.950 | 31.550 | −5.348 | 1.00 | 56.61 | F |
| ATOM | 21741 | OH2 | TIP | F | 458 | 58.332 | 33.171 | −1.901 | 1.00 | 51.71 | F |
| ATOM | 21742 | OH2 | TIP | F | 459 | 44.146 | 22.790 | −27.160 | 1.00 | 37.25 | F |
| ATOM | 21743 | OH2 | TIP | F | 460 | 40.375 | 19.225 | −26.540 | 1.00 | 48.51 | F |
| ATOM | 21744 | OH2 | TIP | F | 461 | 61.542 | 26.045 | −22.682 | 1.00 | 52.88 | F |
| ATOM | 21745 | OH2 | TIP | F | 462 | 61.287 | 14.004 | 7.585 | 1.00 | 44.76 | F |
| ATOM | 21746 | OH2 | TIP | F | 463 | 53.439 | 11.452 | −17.341 | 1.00 | 49.54 | F |
| ATOM | 21747 | OH2 | TIP | F | 464 | 54.372 | 26.132 | −15.089 | 1.00 | 52.38 | F |
| ATOM | 21748 | OH2 | TIP | F | 465 | 41.732 | 27.472 | −1.930 | 1.00 | 55.15 | F |
| ATOM | 21749 | OH2 | TIP | F | 466 | 46.476 | 21.044 | −26.806 | 1.00 | 52.37 | F |
| ATOM | 21750 | OH2 | TIP | F | 467 | 65.795 | 27.507 | −36.714 | 1.00 | 35.74 | F |
| ATOM | 21751 | C1 | NAG | A | 468 | −28.649 | −37.315 | 32.123 | 1.00 | 57.51 | A |
| ATOM | 21752 | C2 | NAG | A | 468 | −29.297 | −37.315 | 30.736 | 1.00 | 57.51 | A |
| ATOM | 21753 | N2 | NAG | A | 468 | −29.620 | −35.952 | 30.358 | 1.00 | 57.51 | A |
| ATOM | 21754 | C7 | NAG | A | 468 | −28.837 | −35.282 | 29.514 | 1.00 | 57.51 | A |
| ATOM | 21755 | O7 | NAG | A | 468 | −27.703 | −35.648 | 29.218 | 1.00 | 57.51 | A |
| ATOM | 21756 | C8 | NAG | A | 468 | −29.399 | −34.012 | 28.911 | 1.00 | 57.51 | A |
| ATOM | 21757 | C3 | NAG | A | 468 | −30.566 | −38.193 | 30.755 | 1.00 | 57.51 | A |
| ATOM | 21758 | O3 | NAG | A | 468 | −31.169 | −38.233 | 29.470 | 1.00 | 57.51 | A |
| ATOM | 21759 | C4 | NAG | A | 468 | −30.202 | −39.615 | 31.206 | 1.00 | 57.51 | A |
| ATOM | 21760 | O4 | NAG | A | 468 | −31.367 | −40.456 | 31.251 | 1.00 | 57.51 | A |
| ATOM | 21761 | C5 | NAG | A | 468 | −29.536 | −39.503 | 32.590 | 1.00 | 57.51 | A |
| ATOM | 21762 | O5 | NAG | A | 468 | −28.350 | −38.662 | 32.511 | 1.00 | 57.51 | A |
| ATOM | 21763 | C6 | NAG | A | 468 | −29.113 | −40.845 | 33.163 | 1.00 | 57.51 | A |
| ATOM | 21764 | O6 | NAG | A | 468 | −27.873 | −41.278 | 32.618 | 1.00 | 57.51 | A |
| ATOM | 21765 | C1 | NAG | A | 469 | −1.059 | −35.497 | 35.906 | 1.00 | 112.81 | A |
| ATOM | 21766 | C2 | NAG | A | 469 | −0.515 | −34.629 | 37.094 | 1.00 | 112.81 | A |
| ATOM | 21767 | N2 | NAG | A | 469 | −0.787 | −33.224 | 36.829 | 1.00 | 112.81 | A |
| ATOM | 21768 | C7 | NAG | A | 469 | 0.219 | −32.360 | 36.668 | 1.00 | 112.81 | A |
| ATOM | 21769 | O7 | NAG | A | 469 | 0.668 | −31.669 | 37.588 | 1.00 | 112.81 | A |
| ATOM | 21770 | C8 | NAG | A | 469 | 0.830 | −32.253 | 35.275 | 1.00 | 112.81 | A |
| ATOM | 21771 | C3 | NAG | A | 469 | 1.010 | −34.773 | 37.356 | 1.00 | 112.81 | A |
| ATOM | 21772 | O3 | NAG | A | 469 | 1.328 | −34.309 | 38.663 | 1.00 | 112.81 | A |
| ATOM | 21773 | C4 | NAG | A | 469 | 1.493 | −36.204 | 37.197 | 1.00 | 112.81 | A |
| ATOM | 21774 | O4 | NAG | A | 469 | 2.910 | −36.252 | 37.294 | 1.00 | 112.81 | A |
| ATOM | 21775 | C5 | NAG | A | 469 | 1.039 | −36.663 | 35.828 | 1.00 | 112.81 | A |
| ATOM | 21776 | O5 | NAG | A | 469 | −0.391 | −36.769 | 35.826 | 1.00 | 112.81 | A |
| ATOM | 21777 | C6 | NAG | A | 469 | 1.593 | −38.002 | 35.402 | 1.00 | 112.81 | A |
| ATOM | 21778 | O6 | NAG | A | 469 | 1.174 | −38.312 | 34.081 | 1.00 | 112.81 | A |
| ATOM | 21779 | C1 | NAG | A | 470 | −50.698 | −29.175 | 43.848 | 1.00 | 153.57 | A |
| ATOM | 21780 | C2 | NAG | A | 470 | −50.893 | −27.722 | 43.400 | 1.00 | 153.57 | A |
| ATOM | 21781 | N2 | NAG | A | 470 | −50.373 | −27.475 | 42.069 | 1.00 | 153.57 | A |
| ATOM | 21782 | C7 | NAG | A | 470 | −50.653 | −26.323 | 41.462 | 1.00 | 153.57 | A |
| ATOM | 21783 | O7 | NAG | A | 470 | −51.796 | −25.996 | 41.147 | 1.00 | 153.57 | A |
| ATOM | 21784 | C8 | NAG | A | 470 | −49.495 | −25.380 | 41.181 | 1.00 | 153.57 | A |
| ATOM | 21785 | C3 | NAG | A | 470 | −50.235 | −26.804 | 44.418 | 1.00 | 153.57 | A |
| ATOM | 21786 | O3 | NAG | A | 470 | −50.446 | −25.450 | 44.040 | 1.00 | 153.57 | A |
| ATOM | 21787 | C4 | NAG | A | 470 | −50.838 | −27.066 | 45.809 | 1.00 | 153.57 | A |
| ATOM | 21788 | O4 | NAG | A | 470 | −50.102 | −26.345 | 46.788 | 1.00 | 153.57 | A |
| ATOM | 21789 | C5 | NAG | A | 470 | −50.825 | −28.568 | 46.167 | 1.00 | 153.57 | A |
| ATOM | 21790 | O5 | NAG | A | 470 | −51.386 | −29.364 | 45.092 | 1.00 | 153.57 | A |
| ATOM | 21791 | C6 | NAG | A | 470 | −51.652 | −28.856 | 47.410 | 1.00 | 153.57 | A |
| ATOM | 21792 | O6 | NAG | A | 470 | −51.272 | −30.083 | 48.015 | 1.00 | 153.57 | A |
| ATOM | 21793 | C1 | NAG | B | 468 | 3.753 | 28.506 | 68.128 | 1.00 | 56.79 | B |
| ATOM | 21794 | C2 | NAG | B | 468 | 2.852 | 27.733 | 69.103 | 1.00 | 56.79 | B |
| ATOM | 21795 | N2 | NAG | B | 468 | 1.596 | 27.424 | 68.443 | 1.00 | 56.79 | B |
| ATOM | 21796 | C7 | NAG | B | 468 | 1.336 | 26.188 | 68.022 | 1.00 | 56.79 | B |
| ATOM | 21797 | O7 | NAG | B | 468 | 2.107 | 25.239 | 68.181 | 1.00 | 56.79 | B |
| ATOM | 21798 | C8 | NAG | B | 468 | 0.012 | 25.967 | 67.312 | 1.00 | 56.79 | B |
| ATOM | 21799 | C3 | NAG | B | 468 | 2.566 | 28.533 | 70.387 | 1.00 | 56.79 | B |
| ATOM | 21800 | O3 | NAG | B | 468 | 1.947 | 27.688 | 71.338 | 1.00 | 56.79 | B |
| ATOM | 21801 | C4 | NAG | B | 468 | 3.849 | 29.108 | 70.983 | 1.00 | 56.79 | B |
| ATOM | 21802 | O4 | NAG | B | 468 | 3.554 | 29.893 | 72.142 | 1.00 | 56.79 | B |
| ATOM | 21803 | C5 | NAG | B | 468 | 4.512 | 29.944 | 69.887 | 1.00 | 56.79 | B |
| ATOM | 21804 | O5 | NAG | B | 468 | 4.887 | 29.076 | 68.799 | 1.00 | 56.79 | B |
| ATOM | 21805 | C6 | NAG | B | 468 | 5.759 | 30.676 | 70.331 | 1.00 | 56.79 | B |
| ATOM | 21806 | O6 | NAG | B | 468 | 6.840 | 29.777 | 70.538 | 1.00 | 56.79 | B |
| ATOM | 21807 | C1 | NAG | B | 469 | 23.782 | 15.210 | 53.924 | 1.00 | 81.08 | B |
| ATOM | 21808 | C2 | NAG | B | 469 | 24.226 | 15.502 | 52.450 | 1.00 | 81.08 | B |
| ATOM | 21809 | N2 | NAG | B | 469 | 23.101 | 15.290 | 51.556 | 1.00 | 81.08 | B |
| ATOM | 21810 | C7 | NAG | B | 469 | 23.222 | 14.493 | 50.495 | 1.00 | 81.08 | B |
| ATOM | 21811 | O7 | NAG | B | 469 | 23.440 | 14.918 | 49.358 | 1.00 | 81.08 | B |
| ATOM | 21812 | C8 | NAG | B | 469 | 23.078 | 12.991 | 50.726 | 1.00 | 81.08 | B |
| ATOM | 21813 | C3 | NAG | B | 469 | 25.410 | 14.632 | 51.953 | 1.00 | 81.08 | B |
| ATOM | 21814 | O3 | NAG | B | 469 | 25.959 | 15.190 | 50.769 | 1.00 | 81.08 | B |
| ATOM | 21815 | C4 | NAG | B | 469 | 26.488 | 14.497 | 53.024 | 1.00 | 81.08 | B |
| ATOM | 21816 | O4 | NAG | B | 469 | 27.528 | 13.627 | 52.586 | 1.00 | 81.08 | B |
| ATOM | 21817 | C5 | NAG | B | 469 | 25.764 | 13.926 | 54.232 | 1.00 | 81.08 | B |
| ATOM | 21818 | O5 | NAG | B | 469 | 24.898 | 14.922 | 54.765 | 1.00 | 81.08 | B |
| ATOM | 21819 | C6 | NAG | B | 469 | 26.602 | 13.458 | 55.384 | 1.00 | 81.08 | B |
| ATOM | 21820 | O6 | NAG | B | 469 | 25.849 | 13.574 | 56.587 | 1.00 | 81.08 | B |
| ATOM | 21821 | C1 | NAG | B | 470 | −13.167 | 48.108 | 64.895 | 1.00 | 124.67 | B |
| ATOM | 21822 | C2 | NAG | B | 470 | −14.394 | 47.327 | 64.348 | 1.00 | 124.67 | B |
| ATOM | 21823 | N2 | NAG | B | 470 | −14.245 | 45.891 | 64.540 | 1.00 | 124.67 | B |
| ATOM | 21824 | C7 | NAG | B | 470 | −15.223 | 45.059 | 64.182 | 1.00 | 124.67 | B |
| ATOM | 21825 | O7 | NAG | B | 470 | −16.407 | 45.282 | 64.424 | 1.00 | 124.67 | B |
| ATOM | 21826 | C8 | NAG | B | 470 | −14.838 | 43.792 | 63.434 | 1.00 | 124.67 | B |
| ATOM | 21827 | C3 | NAG | B | 470 | −14.667 | 47.634 | 62.862 | 1.00 | 124.67 | B |
| ATOM | 21828 | O3 | NAG | B | 470 | −15.967 | 47.162 | 62.525 | 1.00 | 124.67 | B |
| ATOM | 21829 | C4 | NAG | B | 470 | −14.572 | 49.149 | 62.563 | 1.00 | 124.67 | B |
| ATOM | 21830 | O4 | NAG | B | 470 | −14.572 | 49.372 | 61.159 | 1.00 | 124.67 | B |
| ATOM | 21831 | C5 | NAG | B | 470 | −13.286 | 49.712 | 63.160 | 1.00 | 124.67 | B |
| ATOM | 21832 | O5 | NAG | B | 470 | −13.282 | 49.499 | 64.581 | 1.00 | 124.67 | B |
| ATOM | 21833 | C6 | NAG | B | 470 | −13.069 | 51.184 | 62.915 | 1.00 | 124.67 | B |
| ATOM | 21834 | O6 | NAG | B | 470 | −11.772 | 51.413 | 62.386 | 1.00 | 124.67 | B |
| ATOM | 21835 | C1 | NAG | C | 468 | 0.704 | 24.698 | −12.830 | 1.00 | 79.12 | C |
| ATOM | 21836 | C2 | NAG | C | 468 | 0.684 | 26.115 | −12.213 | 1.00 | 79.12 | C |
| ATOM | 21837 | N2 | NAG | C | 468 | −0.292 | 26.174 | −11.148 | 1.00 | 79.12 | C |
| ATOM | 21838 | C7 | NAG | C | 468 | 0.114 | 26.341 | −9.897 | 1.00 | 79.12 | C |
| ATOM | 21839 | O7 | NAG | C | 468 | 1.225 | 25.977 | −9.499 | 1.00 | 79.12 | C |
| ATOM | 21840 | C8 | NAG | C | 468 | −0.862 | 27.016 | −8.947 | 1.00 | 79.12 | C |
| ATOM | 21841 | C3 | NAG | C | 468 | 0.355 | 27.206 | −13.243 | 1.00 | 79.12 | C |
| ATOM | 21842 | O3 | NAG | C | 468 | 0.561 | 28.485 | −12.650 | 1.00 | 79.12 | C |
| ATOM | 21843 | C4 | NAG | C | 468 | 1.240 | 27.054 | −14.490 | 1.00 | 79.12 | C |
| ATOM | 21844 | O4 | NAG | C | 468 | 0.872 | 28.001 | −15.493 | 1.00 | 79.12 | C |
| ATOM | 21845 | C5 | NAG | C | 468 | 1.088 | 25.631 | −15.022 | 1.00 | 79.12 | C |
| ATOM | 21846 | O5 | NAG | C | 468 | 1.523 | 24.687 | −14.017 | 1.00 | 79.12 | C |
| ATOM | 21847 | C6 | NAG | C | 468 | 1.926 | 25.388 | −16.256 | 1.00 | 79.12 | C |
| ATOM | 21848 | O6 | NAG | C | 468 | 3.113 | 24.677 | −15.937 | 1.00 | 79.12 | C |
| ATOM | 21849 | C1 | NAG | C | 469 | 17.518 | 4.112 | −4.003 | 1.00 | 140.88 | C |
| ATOM | 21850 | C2 | NAG | C | 469 | 17.062 | 2.638 | −3.820 | 1.00 | 140.88 | C |
| ATOM | 21851 | N2 | NAG | C | 469 | 15.977 | 2.595 | −2.851 | 1.00 | 140.88 | C |
| ATOM | 21852 | C7 | NAG | C | 469 | 16.162 | 2.078 | −1.635 | 1.00 | 140.88 | C |
| ATOM | 21853 | O7 | NAG | C | 469 | 15.642 | 1.019 | −1.273 | 1.00 | 140.88 | C |
| ATOM | 21854 | C8 | NAG | C | 469 | 17.048 | 2.854 | −0.666 | 1.00 | 140.88 | C |
| ATOM | 21855 | C3 | NAG | C | 469 | 18.203 | 1.702 | −3.349 | 1.00 | 140.88 | C |
| ATOM | 21856 | O3 | NAG | C | 469 | 17.810 | 0.342 | −3.488 | 1.00 | 140.88 | C |
| ATOM | 21857 | C4 | NAG | C | 469 | 19.487 | 1.941 | −4.137 | 1.00 | 140.88 | C |
| ATOM | 21858 | O4 | NAG | C | 469 | 20.544 | 1.166 | −3.583 | 1.00 | 140.88 | C |
| ATOM | 21859 | C5 | NAG | C | 469 | 19.818 | 3.427 | −4.055 | 1.00 | 140.88 | C |
| ATOM | 21860 | O5 | NAG | C | 469 | 18.774 | 4.180 | −4.697 | 1.00 | 140.88 | C |
| ATOM | 21861 | C6 | NAG | C | 469 | 21.125 | 3.808 | −4.720 | 1.00 | 140.88 | C |
| ATOM | 21862 | O6 | NAG | C | 469 | 21.569 | 5.084 | −4.278 | 1.00 | 140.88 | C |
| ATOM | 21863 | C1 | NAG | C | 470 | −24.264 | 27.981 | −20.130 | 1.00 | 147.99 | C |
| ATOM | 21864 | C2 | NAG | C | 470 | −25.086 | 28.340 | −18.862 | 1.00 | 147.99 | C |
| ATOM | 21865 | N2 | NAG | C | 470 | −24.216 | 28.803 | −17.793 | 1.00 | 147.99 | C |
| ATOM | 21866 | C7 | NAG | C | 470 | −24.695 | 28.982 | −16.561 | 1.00 | 147.99 | C |
| ATOM | 21867 | O7 | NAG | C | 470 | −25.498 | 29.873 | −16.277 | 1.00 | 147.99 | C |
| ATOM | 21868 | C8 | NAG | C | 470 | −24.221 | 28.022 | −15.479 | 1.00 | 147.99 | C |
| ATOM | 21869 | C3 | NAG | C | 470 | −25.945 | 27.163 | −18.365 | 1.00 | 147.99 | C |
| ATOM | 21870 | O3 | NAG | C | 470 | −26.886 | 27.639 | −17.409 | 1.00 | 147.99 | C |
| ATOM | 21871 | C4 | NAG | C | 470 | −26.698 | 26.496 | −19.521 | 1.00 | 147.99 | C |
| ATOM | 21872 | O4 | NAG | C | 470 | −27.326 | 25.307 | −19.064 | 1.00 | 147.99 | C |
| ATOM | 21873 | C5 | NAG | C | 470 | −25.728 | 26.159 | −20.652 | 1.00 | 147.99 | C |
| ATOM | 21874 | O5 | NAG | C | 470 | −25.097 | 27.367 | −21.127 | 1.00 | 147.99 | C |
| ATOM | 21875 | C6 | NAG | C | 470 | −26.412 | 25.504 | −21.836 | 1.00 | 147.99 | C |
| ATOM | 21876 | O6 | NAG | C | 470 | −26.152 | 24.109 | −21.870 | 1.00 | 147.99 | C |
| ATOM | 21877 | C1 | NAG | D | 468 | 47.479 | 32.389 | −67.588 | 1.00 | 49.52 | D |
| ATOM | 21878 | C2 | NAG | D | 468 | 48.138 | 31.547 | −68.690 | 1.00 | 49.52 | D |
| ATOM | 21879 | N2 | NAG | D | 468 | 49.424 | 31.075 | −68.205 | 1.00 | 49.52 | D |
| ATOM | 21880 | C7 | NAG | D | 468 | 49.547 | 29.878 | −67.633 | 1.00 | 49.52 | D |
| ATOM | 21881 | O7 | NAG | D | 468 | 48.652 | 29.036 | −67.630 | 1.00 | 49.52 | D |
| ATOM | 21882 | C8 | NAG | D | 468 | 50.877 | 29.567 | −66.964 | 1.00 | 49.52 | D |
| ATOM | 21883 | C3 | NAG | D | 468 | 48.352 | 32.356 | −69.989 | 1.00 | 49.52 | D |
| ATOM | 21884 | O3 | NAG | D | 468 | 48.780 | 31.508 | −71.039 | 1.00 | 49.52 | D |
| ATOM | 21885 | C4 | NAG | D | 468 | 47.074 | 33.070 | −70.407 | 1.00 | 49.52 | D |
| ATOM | 21886 | O4 | NAG | D | 468 | 47.299 | 33.841 | −71.596 | 1.00 | 49.52 | D |
| ATOM | 21887 | C5 | NAG | D | 468 | 46.694 | 33.956 | −69.231 | 1.00 | 49.52 | D |
| ATOM | 21888 | O5 | NAG | D | 468 | 46.358 | 33.119 | −68.101 | 1.00 | 49.52 | D |
| ATOM | 21889 | C6 | NAG | D | 468 | 45.525 | 34.889 | −69.499 | 1.00 | 49.52 | D |
| ATOM | 21890 | O6 | NAG | D | 468 | 44.276 | 34.223 | −69.383 | 1.00 | 49.52 | D |
| ATOM | 21891 | C1 | NAG | D | 469 | 28.308 | 20.674 | −50.916 | 1.00 | 96.43 | D |
| ATOM | 21892 | C2 | NAG | D | 469 | 27.862 | 21.124 | −49.490 | 1.00 | 96.43 | D |
| ATOM | 21893 | N2 | NAG | D | 469 | 28.925 | 20.827 | −48.547 | 1.00 | 96.43 | D |
| ATOM | 21894 | C7 | NAG | D | 469 | 28.802 | 19.791 | −47.714 | 1.00 | 96.43 | D |
| ATOM | 21895 | O7 | NAG | D | 469 | 28.198 | 19.860 | −46.639 | 1.00 | 96.43 | D |
| ATOM | 21896 | C8 | NAG | D | 469 | 29.419 | 18.470 | −48.142 | 1.00 | 96.43 | D |
| ATOM | 21897 | C3 | NAG | D | 469 | 26.588 | 20.432 | −48.951 | 1.00 | 96.43 | D |
| ATOM | 21898 | O3 | NAG | D | 469 | 26.006 | 21.222 | −47.920 | 1.00 | 96.43 | D |
| ATOM | 21899 | C4 | NAG | D | 469 | 25.543 | 20.143 | −50.023 | 1.00 | 96.43 | D |
| ATOM | 21900 | O4 | NAG | D | 469 | 24.564 | 19.254 | −49.495 | 1.00 | 96.43 | D |
| ATOM | 21901 | C5 | NAG | D | 469 | 26.242 | 19.508 | −51.227 | 1.00 | 96.43 | D |
| ATOM | 21902 | O5 | NAG | D | 469 | 27.179 | 20.449 | −51.771 | 1.00 | 96.43 | D |
| ATOM | 21903 | C6 | NAG | D | 469 | 25.310 | 19.090 | −52.357 | 1.00 | 96.43 | D |
| ATOM | 21904 | O6 | NAG | D | 469 | 25.786 | 19.540 | −53.618 | 1.00 | 96.43 | D |
| ATOM | 21905 | C1 | NAG | D | 470 | 65.895 | 50.746 | −66.573 | 1.00 | 151.66 | D |
| ATOM | 21906 | C2 | NAG | D | 470 | 67.062 | 49.973 | −65.912 | 1.00 | 151.66 | D |
| ATOM | 21907 | N2 | NAG | D | 470 | 66.880 | 48.539 | −66.090 | 1.00 | 151.66 | D |
| ATOM | 21908 | C7 | NAG | D | 470 | 67.821 | 47.676 | −65.706 | 1.00 | 151.66 | D |
| ATOM | 21909 | O7 | NAG | D | 470 | 68.948 | 47.650 | −66.202 | 1.00 | 151.66 | D |
| ATOM | 21910 | C8 | NAG | D | 470 | 67.459 | 46.688 | −64.607 | 1.00 | 151.66 | D |
| ATOM | 21911 | C3 | NAG | D | 470 | 67.170 | 50.311 | −64.411 | 1.00 | 151.66 | D |
| ATOM | 21912 | O3 | NAG | D | 470 | 68.399 | 49.806 | −63.901 | 1.00 | 151.66 | D |
| ATOM | 21913 | C4 | NAG | D | 470 | 67.100 | 51.834 | −64.164 | 1.00 | 151.66 | D |
| ATOM | 21914 | O4 | NAG | D | 470 | 66.968 | 52.092 | −62.773 | 1.00 | 151.66 | D |
| ATOM | 21915 | C5 | NAG | D | 470 | 65.903 | 52.430 | −64.908 | 1.00 | 151.66 | D |
| ATOM | 21916 | O5 | NAG | D | 470 | 66.023 | 52.147 | −66.314 | 1.00 | 151.66 | D |
| ATOM | 21917 | C6 | NAG | D | 470 | 65.748 | 53.927 | −64.754 | 1.00 | 151.66 | D |
| ATOM | 21918 | O6 | NAG | D | 470 | 64.432 | 54.335 | −65.100 | 1.00 | 151.66 | D |
| ATOM | 21919 | C1 | NAG | E | 468 | 82.012 | −33.741 | −35.940 | 1.00 | 54.79 | E |
| ATOM | 21920 | C2 | NAG | E | 468 | 82.857 | −33.812 | −34.660 | 1.00 | 54.79 | E |
| ATOM | 21921 | N2 | NAG | E | 468 | 83.299 | −32.477 | −34.289 | 1.00 | 54.79 | E |
| ATOM | 21922 | C7 | NAG | E | 468 | 82.646 | −31.766 | −33.360 | 1.00 | 54.79 | E |
| ATOM | 21923 | O7 | NAG | E | 468 | 81.591 | −32.129 | −32.818 | 1.00 | 54.79 | E |
| ATOM | 21924 | C8 | NAG | E | 468 | 83.266 | −30.433 | −32.978 | 1.00 | 54.79 | E |
| ATOM | 21925 | C3 | NAG | E | 468 | 84.065 | −34.751 | −34.839 | 1.00 | 54.79 | E |
| ATOM | 21926 | O3 | NAG | E | 468 | 84.721 | −34.920 | −33.595 | 1.00 | 54.79 | E |
| ATOM | 21927 | C4 | NAG | E | 468 | 83.593 | −36.115 | −35.332 | 1.00 | 54.79 | E |
| ATOM | 21928 | O4 | NAG | E | 468 | 84.702 | −37.005 | −35.524 | 1.00 | 54.79 | E |
| ATOM | 21929 | C5 | NAG | E | 468 | 82.812 | −35.910 | −36.632 | 1.00 | 54.79 | E |
| ATOM | 21930 | O5 | NAG | E | 468 | 81.662 | −35.063 | −36.387 | 1.00 | 54.79 | E |
| ATOM | 21931 | C6 | NAG | E | 468 | 82.295 | −37.212 | −37.223 | 1.00 | 54.79 | E |
| ATOM | 21932 | O6 | NAG | E | 468 | 81.254 | −37.772 | −36.431 | 1.00 | 54.79 | E |
| ATOM | 21933 | C1 | NAG | E | 469 | 54.129 | −32.392 | −35.969 | 1.00 | 122.52 | E |
| ATOM | 21934 | C2 | NAG | E | 469 | 53.443 | −31.462 | −37.018 | 1.00 | 122.52 | E |
| ATOM | 21935 | N2 | NAG | E | 469 | 53.780 | −30.075 | −36.737 | 1.00 | 122.52 | E |
| ATOM | 21936 | C7 | NAG | E | 469 | 52.843 | −29.208 | −36.344 | 1.00 | 122.52 | E |
| ATOM | 21937 | O7 | NAG | E | 469 | 52.274 | −28.434 | −37.121 | 1.00 | 122.52 | E |
| ATOM | 21938 | C8 | NAG | E | 469 | 52.483 | −29.189 | −34.862 | 1.00 | 122.52 | E |
| ATOM | 21939 | C3 | NAG | E | 469 | 51.904 | −31.615 | −37.041 | 1.00 | 122.52 | E |
| ATOM | 21940 | O3 | NAG | E | 469 | 51.354 | −30.948 | −38.169 | 1.00 | 122.52 | E |
| ATOM | 21941 | C4 | NAG | E | 469 | 51.506 | −33.076 | −37.077 | 1.00 | 122.52 | E |
| ATOM | 21942 | O4 | NAG | E | 469 | 50.091 | −33.194 | −37.048 | 1.00 | 122.52 | E |
| ATOM | 21943 | C5 | NAG | E | 469 | 52.122 | −33.727 | −35.857 | 1.00 | 122.52 | E |
| ATOM | 21944 | O5 | NAG | E | 469 | 53.550 | −33.708 | −35.985 | 1.00 | 122.52 | E |
| ATOM | 21945 | C6 | NAG | E | 469 | 51.731 | −35.169 | −35.685 | 1.00 | 122.52 | E |
| ATOM | 21946 | O6 | NAG | E | 469 | 52.835 | −35.918 | −35.201 | 1.00 | 122.52 | E |
| ATOM | 21947 | C1 | NAG | E | 470 | 102.230 | −25.224 | −50.276 | 1.00 | 158.03 | E |
| ATOM | 21948 | C2 | NAG | E | 470 | 102.652 | −23.930 | −49.540 | 1.00 | 158.03 | E |
| ATOM | 21949 | N2 | NAG | E | 470 | 102.132 | −23.908 | −48.183 | 1.00 | 158.03 | E |
| ATOM | 21950 | C7 | NAG | E | 470 | 102.473 | −22.929 | −47.345 | 1.00 | 158.03 | E |
| ATOM | 21951 | O7 | NAG | E | 470 | 103.612 | −22.800 | −46.899 | 1.00 | 158.03 | E |
| ATOM | 21952 | C8 | NAG | E | 470 | 101.390 | −21.938 | −46.943 | 1.00 | 158.03 | E |
| ATOM | 21953 | C3 | NAG | E | 470 | 102.192 | −22.680 | −50.305 | 1.00 | 158.03 | E |
| ATOM | 21954 | O3 | NAG | E | 470 | 102.787 | −21.525 | −49.724 | 1.00 | 158.03 | E |
| ATOM | 21955 | C4 | NAG | E | 470 | 102.593 | −22.771 | −51.783 | 1.00 | 158.03 | E |
| ATOM | 21956 | O4 | NAG | E | 470 | 102.025 | −21.687 | −52.505 | 1.00 | 158.03 | E |
| ATOM | 21957 | C5 | NAG | E | 470 | 102.106 | −24.093 | −52.378 | 1.00 | 158.03 | E |
| ATOM | 21958 | O5 | NAG | E | 470 | 102.666 | −25.199 | −51.642 | 1.00 | 158.03 | E |
| ATOM | 21959 | C6 | NAG | E | 470 | 102.516 | −24.265 | −53.823 | 1.00 | 158.03 | E |
| ATOM | 21960 | O6 | NAG | E | 470 | 102.339 | −25.608 | −54.242 | 1.00 | 158.03 | E |
| ATOM | 21961 | C1 | NAG | F | 468 | 59.178 | 27.704 | 12.879 | 1.00 | 90.14 | F |
| ATOM | 21962 | C2 | NAG | F | 468 | 59.308 | 29.123 | 12.317 | 1.00 | 90.14 | F |
| ATOM | 21963 | N2 | NAG | F | 468 | 60.050 | 29.106 | 11.073 | 1.00 | 90.14 | F |
| ATOM | 21964 | C7 | NAG | F | 468 | 59.414 | 29.309 | 9.927 | 1.00 | 90.14 | F |
| ATOM | 21965 | O7 | NAG | F | 468 | 58.190 | 29.211 | 9.815 | 1.00 | 90.14 | F |
| ATOM | 21966 | C8 | NAG | F | 468 | 60.265 | 29.678 | 8.725 | 1.00 | 90.14 | F |
| ATOM | 21967 | C3 | NAG | F | 468 | 60.006 | 30.022 | 13.346 | 1.00 | 90.14 | F |
| ATOM | 21968 | O3 | NAG | F | 468 | 60.083 | 31.353 | 12.849 | 1.00 | 90.14 | F |
| ATOM | 21969 | C4 | NAG | F | 468 | 59.219 | 29.987 | 14.668 | 1.00 | 90.14 | F |
| ATOM | 21970 | O4 | NAG | F | 468 | 59.876 | 30.764 | 15.668 | 1.00 | 90.14 | F |
| ATOM | 21971 | C5 | NAG | F | 468 | 59.068 | 28.529 | 15.137 | 1.00 | 90.14 | F |
| ATOM | 21972 | O5 | NAG | F | 468 | 58.433 | 27.733 | 14.106 | 1.00 | 90.14 | F |
| ATOM | 21973 | C6 | NAG | F | 468 | 58.201 | 28.407 | 16.374 | 1.00 | 90.14 | F |
| ATOM | 21974 | O6 | NAG | F | 468 | 57.181 | 27.432 | 16.189 | 1.00 | 90.14 | F |
| ATOM | 21975 | C1 | NAG | F | 469 | 40.547 | 8.020 | 5.881 | 1.00 | 128.78 | F |
| ATOM | 21976 | C2 | NAG | F | 469 | 40.967 | 6.526 | 5.740 | 1.00 | 128.78 | F |
| ATOM | 21977 | N2 | NAG | F | 469 | 41.939 | 6.409 | 4.660 | 1.00 | 128.78 | F |
| ATOM | 21978 | C7 | NAG | F | 469 | 41.607 | 5.882 | 3.479 | 1.00 | 128.78 | F |
| ATOM | 21979 | O7 | NAG | F | 469 | 42.038 | 4.795 | 3.095 | 1.00 | 128.78 | F |
| ATOM | 21980 | C8 | NAG | F | 469 | 40.675 | 6.679 | 2.574 | 1.00 | 128.78 | F |
| ATOM | 21981 | C3 | NAG | F | 469 | 39.785 | 5.557 | 5.475 | 1.00 | 128.78 | F |
| ATOM | 21982 | O3 | NAG | F | 469 | 40.185 | 4.220 | 5.744 | 1.00 | 128.78 | F |
| ATOM | 21983 | C4 | NAG | F | 469 | 38.575 | 5.893 | 6.333 | 1.00 | 128.78 | F |
| ATOM | 21984 | O4 | NAG | F | 469 | 37.475 | 5.068 | 5.974 | 1.00 | 128.78 | F |
| ATOM | 21985 | C5 | NAG | F | 469 | 38.243 | 7.351 | 6.094 | 1.00 | 128.78 | F |
| ATOM | 21986 | O5 | NAG | F | 469 | 39.313 | 8.160 | 6.614 | 1.00 | 128.78 | F |
| ATOM | 21987 | C6 | NAG | F | 469 | 36.958 | 7.795 | 6.763 | 1.00 | 128.78 | F |
| ATOM | 21988 | O6 | NAG | F | 469 | 36.152 | 8.549 | 5.869 | 1.00 | 128.78 | F |
| ATOM | 21989 | C1 | NAG | F | 470 | 84.705 | 30.138 | 16.795 | 1.00 | 150.52 | F |
| ATOM | 21990 | C2 | NAG | F | 470 | 85.213 | 30.232 | 15.342 | 1.00 | 150.52 | F |
| ATOM | 21991 | N2 | NAG | F | 470 | 84.113 | 30.447 | 14.415 | 1.00 | 150.52 | F |
| ATOM | 21992 | C7 | NAG | F | 470 | 84.352 | 30.588 | 13.113 | 1.00 | 150.52 | F |
| ATOM | 21993 | O7 | NAG | F | 470 | 85.127 | 31.432 | 12.660 | 1.00 | 150.52 | F |
| ATOM | 21994 | C8 | NAG | F | 470 | 83.630 | 29.641 | 12.170 | 1.00 | 150.52 | F |
| ATOM | 21995 | C3 | NAG | F | 470 | 85.990 | 28.963 | 14.974 | 1.00 | 150.52 | F |
| ATOM | 21996 | O3 | NAG | F | 470 | 86.567 | 29.111 | 13.684 | 1.00 | 150.52 | F |
| ATOM | 21997 | C4 | NAG | F | 470 | 87.094 | 28.703 | 16.010 | 1.00 | 150.52 | F |
| ATOM | 21998 | O4 | NAG | F | 470 | 87.716 | 27.456 | 15.741 | 1.00 | 150.52 | F |
| ATOM | 21999 | C5 | NAG | F | 470 | 86.497 | 28.689 | 17.426 | 1.00 | 150.52 | F |
| ATOM | 22000 | O5 | NAG | F | 470 | 85.813 | 29.934 | 17.686 | 1.00 | 150.52 | F |
| ATOM | 22001 | C6 | NAG | F | 470 | 87.532 | 28.498 | 18.520 | 1.00 | 150.52 | F |
| ATOM | 22002 | O6 | NAG | F | 470 | 87.240 | 27.350 | 19.308 | 1.00 | 150.52 | F |
| END | | | | | | | | | | | |

1. A method of creating a therapeutic candidate compound comprising:
a) providing a 2L polypeptide of SEQ ID No. 1, SEQ ID No. 2, or SEQ ID No. 3;
b) modifying at least one amino acid of the 2L polypeptide at a position other than the 2L interface amino acids HIS58, GLU99, GLY101, PHE104, LYS125, GLY157, TYR160, MET161, GLY162, LEU165, LEU168, GLN171, LYS175, GLU225, VAL229, ASN230, GLY231, ARG232, or GLU289 for SEQ ID No.'s 1-2 or HIS57, GLU100, GLY102, PHE105, LYS126, GLY157, TYR161, ALA162, GLY163, LEU166, PHE169, GLN172, LYS176, GLU226, VAL230, ASN231, GLY232, ARG233, or GLU290 for SEQ ID No. 3, to form a candidate compound; and,
c) purifying the candidate compound.

2. A method of creating a therapeutic candidate compound comprising:
a) providing an immature sequence comprising a 2L polypeptide of SEQ ID No. 1, SEQ ID No. 2, or SEQ ID No. 3 and an identifiable signal sequence;
b) modifying at least one amino acid of the 2L polypeptide at a position other than HIS58, GLU99, GLY101, PHE104, LYS125, GLY157, TYR160, MET161, GLY162, LEU165, LEU168, GLN171, LYS175, GLU225, VAL229, ASN230, GLY231, ARG232, or GLU289 for SEQ ID No.'s 1-2 or at a position other than HIS57, GLU100, GLY102, PHE105, LYS126, GLY157, TYR161, ALA162, GLY163, LEU166, PHE169, GLN172, LYS176, GLU226, VAL230, ASN231, GLY232, ARG233, or GLU290 for SEQ ID No. 3, to form a sequence of a candidate compound;
c) expressing the sequence of the candidate compound from a host cell to create a secreted form of the candidate compound; and,
d) purifying the candidate compound.

3. The method according to claim 1, wherein the method further comprises verifying that the candidate compound binds to human TNFα with a binding affinity K_{d} of from about 40 picomoles to about 440 picomoles.

4. A method of creating a therapeutic candidate compound comprising:
a) providing a 2L polypeptide of SEQ ID No. 1, SEQ ID No. 2, or SEQ ID No. 3;
b) modifying at least one amino acid of the 2L polypeptide at a position selected from the group consisting of HIS58, GLU99, GLY101, PHE104, LYS125, GLY157, TYR160, MET161, GLY162, LEU165, LEU168, GLN171, LYS175, GLU225, VAL229, ASN230, GLY231, ARG232, and GLU289 for SEQ ID No.'s 1-2 or at a position selected from the group consisting of HIS57, GLU100, GLY102, PHE105, LYS126, GLY157, TYR161, ALA162, GLY163, LEU166, PHE169, GLN172, LYS176, GLU226, VAL230, ASN231, GLY232, ARG233, and GLU290 for SEQ ID No. 3, to form a candidate compound; and,
c) purifying the candidate compound.

5. A method of creating a therapeutic candidate compound comprising:
a) providing an immature sequence comprising a 2L polypeptide of SEQ ID No. 1, SEQ ID No. 2, or SEQ ID No. 3 and an identifiable signal sequence;
b) modifying at least one amino acid of the 2L polypeptide at a position selected from the group consisting of HIS58, GLU99, GLY101, PHE104, LYS125, GLY157, TYR160, MET161, GLY162, LEU165, LEU168, GLN171, LYS175, GLU225, VAL229, ASN230, GLY231, ARG232, and GLU289 for SEQ ID No.'s 1-2 or at a position selected from the group consisting of HIS57, GLU100, GLY102, PHE105, LYS126, GLY157, TYR161, ALA162, GLY163, LEU166, PHE169, GLN172, LYS176, GLU226, VAL230, ASN231, GLY232, ARG233, and GLU290 for SEQ ID No. 3, to form a sequence of a candidate compound;
c) expressing the sequence of the candidate compound from a host cell to create a secreted form of the candidate compound; and,
d) purifying the candidate compound.

6. The method according to claim 4, wherein the method further comprises verifying that the candidate compound binds to human TNFα with a binding affinity K_{d} less than that of the 2L polypeptide.

7. The method according to claim 2, wherein the identifiable signal sequence is an N-terminal signal sequence.

8. The method according to claim 2, wherein the immature sequence is according to SEQ ID No. 15, SEQ ID No. 16, or SEQ ID No.

9. The method according to claim 1, wherein the method further comprises confirming in vitro that the candidate compound suppresses signaling via the TNFα cascade.

10. The method according to claim 1, wherein the modification of the at least one amino acid is selected from substitution, addition and deletion.

11. The method according to claim 1, wherein the method further comprises confirming in vitro that the candidate compound competitively inhibits human TNFR1 or human TNFR2 from binding to human TNFα.

12. A method for determining whether or not a test compound is a therapeutic candidate compound for use in treating a TNFα mediated disease or condition in humans, the method comprising:
a) providing the test compound or an electronic representation of the test compound;
b) determining whether the test compound or electronic representation of the test compound binds to at least TNFα interface amino acids TYR87, ARG31, ARG32, ASN30, SER86, ARG82, GLU127, ASN34, VAL91, ALA33, VAL91, GLN21, ARG44, GLU42, LEU37, LEU43, VAL41 and GLN27 of hTNFα, or competitively inhibits binding of hTNFR1 or hTNFR2 to the TNFα interface amino acids, to within a specified confidence interval;
c) for test compounds that bind to the TNFα interface amino acids or competitively inhibit binding of human TNFR1 or human TNFR2 to the TNFα interface amino acids, confirming in vitro that the test compound suppresses signaling via the TNFα cascade, thereby rendering it a therapeutic candidate compound.

13. The method according to claim 1, wherein the method further comprises verifying that the candidate compound binds to human TNFα with a shape complementarity index of from 0.64 to 0.78.

14. The method according to claim 1, wherein the method further comprises verifying that the candidate compound, upon binding to human TNFα, imparts a change in conformation of human TNFα at GLN21, ALA22, GLU23, GLY24, GLN25, ARG31, ARG32, ALA33, ASN34, ALA35, PHE144, ALA145, GLU146, SER147, and GLY148.

15. The method according to claim 1, wherein the method further comprises verifying that the modification reduces immunogenicity of the candidate compound as compared with the 2L polypeptide of SEQ ID Nos. 1, 2 or 3.

16. The method according to claim 1, wherein the method further comprises verifying that the modification alters the circulating half life of the candidate compound as compared with the 2L polypeptide of SEQ ID Nos. 1, 2 or 3.

17. The method according to claim 1, wherein the method further comprises verifying that the candidate compound binds to human TNFα over a surface area of at least 2000 Å².

18. A therapeutic candidate compound obtained according to the method of claim 1.

19. A method of treating a TNFα mediated disease or condition in humans comprising administering a therapeutic candidate compound according to claim 18 in an amount and for a time sufficient to achieve a therapeutic effect.

20. Use of a therapeutic candidate compound according to claim 19 in an amount and for a time sufficient to achieve a therapeutic effect in treating a TNFα mediated disease or condition in humans.

21. Use of a therapeutic candidate compound according to claim 19 in the manufacture of a medicament for treating a TNFα mediated disease or condition.

22. A pharmaceutical composition comprising a purified therapeutic candidate compound according to claim 19 and a pharmaceutically acceptable diluent formulated for administration to a patient suffering from or at risk of developing a TNFα mediated disease or condition.

23. A purified nucleic acid molecule that encodes a polypeptide comprising the sequence of the candidate compound of claim 2.

24. A purified nucleic acid molecule that hybridizes under high stringency conditions to the nucleic acid molecule of claim 21.

25. A vector comprising the nucleic acid molecule of claim 23.

26. A host cell comprising the vector of claim 25.

27. A crystal of the secreted form of Yaba-like disease virus 2L protein bound to human TNFα.

28. The crystal of claim 27, wherein the Yaba-like disease virus 2L protein comprises an amino acid sequence according to SEQ ID No. 1.

29. The crystal of claim 27, wherein the crystal has a three-dimensional structure as defined by the atomic coordinates of Table 4 with a root mean square deviation of 1.0 Å for each atomic coordinate.


SEQUENCE LISTING (18 sequences)

SEQ ID NO: 1 - protein, 322 aa, Yaba-like Disease Virus
ITLKYNYTVTLKDDGLYDGVFYDHYNDQLVTKISYNHETRHGNVNFRADWFNISRSPHTPGNDYNFNFWYSLMKETLEEINKNDSTKTTSLSLITGCYETGLLFGSYGYVETANGPLARYHTGDKRFTKMTHKGFPKVGMLTVKNTLWKDVKAYLGGFEYMGCSLAILDYQKMAKGKIPKDTTPTVKVTGNELEDGNMTLECTVNSFYPPDVITKWIESEHFKGEYKYVNGRYYPEWGRKSNYEPGEPGFPWNIKKDKDANTYSLTDLVRTTSKMSSQPVCVVFHDTLEAQVYTCSEGCNGELYDHLYRKTEEGEGEEDEED

SEQ ID NO: 2 - protein, 322 aa, Tanapox Virus
ITLKYNYTVTLKDNGLYDGVFYDHYNDQLVTKISYNHETRHGNVNFRADWFNISRSPHTPGNDYNFNFWYSLMKETLEEINKNDSTKTTSLSLITGCYETGLLFGSYGYVETANGPLARYHTGDKRFTKMTHKGFPKVGMLTVKNTLWKDVKTYLGGFEYMGCSLAILDYQKMAKGEIPKDTTPTVKVTGNELEDGNMTLECSVNSFYPPDVITKWIESEHFKGEYKYVNGRYYPEWGRKSDYEPGEPGFPWNIKKDKDANTYSLTDLVRTTSKMSSQLVCVVFHDTLEAQVYTCSEGCNGELYDHLYRKTEEGEGEEDEED

SEQ ID NO: 3 - protein, 233 aa, Yaba monkey tumor virus
ITLRYNYTVTVKNGLYDGVFFDYYNDQLVTRISYNHETRHGNVNSRASWFDISKSPHTPGDDYHFNFWYPLMKDTLESINSNKNESDKCSSLSLILGCYETGSLFGSYGYVESSGGPLARYSTKDKKFLKMTDKGFPKVGMLTVHGPSWQTVKKYVGGFVYAGCLLAIFDYQKMAKNNIPSNVMPTVTVTGEELQDGNTTLKCNVKSFYPPDVMIKWIESKYFNGEYRYVNGR

SEQ ID NO: 4 - DNA, 1017 bp, Yaba-like Disease Virus
atggataagttactattatttagcacaattgtagcagtttgtaactgcataactttaaaatataattatactgttacgttaaaagatgatgggttatacgatggagtattttacgatcattacaacgatcagttagtgacgaaaatatcatataaccatgaaactagacacggaaacgtaaattttagggctgattggtttaatatttctaggagtccccacacgccaggtaacgattataactttaacttttggtattctttaatgaaagaaactttagaagaaattaataaaaacgatagcacaaaaactacttcgctttcattaatcactgggtgttatgaaacaggattattatttggtagttatgggtatgtagaaacggccaacgggccgttggccagataccatacaggagataaaaggtttacgaaaatgacacataaaggttttcccaaggttggaatgttaactgtaaaaaacactctttggaaagatgtaaaagcttatttaggcggttttgaatatatgggatgttcattagctattttagattaccaaaaaatggctaaaggtaaaataccaaaagatacaacacctacagtgaaagtaacgggtaatgagttagaagatggtaacatgactcttgaatgcactgtaaattcattttaccctcctgacgtaattactaagtggatagaaagcgaacattttaaaggtgaatataaatatgttaacggaagatactatccagaatgggggagaaaatccaattatgagccaggagagccaggttttccatggaatatcaaaaaagataaagatgcaaatacatatagtttaacagatttagtacgtacaacatcaaaaatgagtagtcaaccagtatgtgttgttttccatgacactttagaagcgcaagtttatacttgttctgaaggatgcaatggagagctatacgatcacctatatagaaaaacagaagaaggggaaggtgaagaggatgaagaagactga

SEQ ID NO: 5 - DNA, 1017 bp, Tanapox Virus
atgaataagttaatattatttagcacaattgtagcagtttgtaactgcataactttaaaatataattatactgttacgttaaaagataatgggttatacgatggagtattttacgatcattacaacgatcagttagtaacgaaaatatcatataaccacgaaactagacacggaaacgtaaattttagggctgattggtttaatatttctaggagtccccacacgccaggtaacgattacaactttaacttttggtattctttaatgaaagaaactttagaagaaattaataaaaacgatagcacaaaaactacttcgctttcattaatcactgggtgttatgaaacaggattattatttggtagttatgggtatgtagaaacggccaacggaccgttggccagataccatacaggagataaaaggtttacgaaaatgacacataaaggttttcccaaggttggaatgttaactgtaaaaaacactctttggaaagatgtaaaaacttatctaggcggttttgaatacatgggatgttcattagctattttagattaccaaaaaatggctaaaggtgaaataccaaaagatacaacacctacagtgaaagtaacgggtaatgagttagaagatggtaacatgactcttgaatgcagtgtaaattcattttaccctcctgacgtaattactaagtggatagaaagcgaacattttaaaggtgaatataaatatgttaacggaagatactatccagaatgggggagaaaatccgattatgagccaggagagccaggttttccatggaatattaaaaaagataaagatgcaaacacatatagtttaacagatttagtacgtacaacatcaaaaatgagtagtcaactagtatgtgttgttttccatgacactttagaagcgcaagtttatacttgttctgaaggatgcaatggagagctatacgaccacctatatagaaaaacagaagaaggagaaggtgaagaggatgaagaagactga

SEQ ID NO: 6 - DNA, 1026 bp, Yaba monkey tumor virus
atgaataagttaattttatcgttgttgggttttgtggcaacttgcaattgtataaccttaagatataattataccgttacggtaaagaatggattatacgacggggtattttttgattattacaacgatcagttagtaacgaggatatcatataaccatgaaactagacacggaaacgtaaattctagagcttcatggtttgatatctctaaaagccctcatactccgggtgacgattaccactttaacttttggtacccgttaatgaaagatactttggagtccatcaatagtaataaaaacgaaagcgataaatgttcttcgttgtcgttaattttggggtgttatgaaacgggatctctttttgggagttacggatacgttgagtcaagtggcggaccgttggctaggtatagcacgaaagataaaaagtttttaaaaatgacagataaaggatttccaaaggttggaatgttaaccgttcatggtcctagttggcaaacagttaaaaaatacgtgggagggtttgtgtacgctggatgtttgctagctatttttgattatcaaaaaatggctaagaataacatacctagtaatgtaatgccaactgttacggtaacgggtgaggaactgcaagatggtaacacaacgcttaagtgtaacgtaaaatctttttaccctccagacgtaatgatcaagtggatagaaagtaaatattttaacggtgaatatagatacgttaatggaagagaatacccggaatggggaaggcaatcagattatgagcccggagagccaggttttccgttacatccaaaaaaagatgacggtaaaaccacttacagccttttagattttggtcgcactacgtcaggattaactagtcagttagtttgtgttgttttccatgacacgtttgaatcgcaggttaatacatgttccgaagggtgtgaaggtaaattatacgatcacctatatagaaaatcggaagaaggagacgaggttgtggaggacgaagaagactga

SEQ ID NO: 7 - DNA, 27 bp, Artificial Sequence
  2L forward primer
catggataagttactattatttagcac

SEQ ID NO: 8 - DNA, 28 bp, Artificial Sequence
  2L reverse primer
tcagtcttcttcatcctcttcaccttcc

SEQ ID NO: 9 - DNA, 42 bp, Artificial Sequence
  HIS-2L forward primer
cgcggatcccctataaatatggataagttactattatttagc

SEQ ID NO: 10 - DNA, 39 bp, Artificial Sequence
  HIS-2L reverse primer
ccgctcgagaccttctccttcttctgtttttctatatag

SEQ ID NO: 11 - DNA, 486 bp, Homo sapiens
atgggcagccatcatcatcatcatcaccgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttggcgagtctgggcaggtctactttgggatcattgccctgtga

SEQ ID NO: 12 - DNA, 55 bp, Artificial Sequence
  HIS-TNF forward primer
gaccatatgggcagccatcatcatcatcatcaccgaaccccgagtgacaagcctg

SEQ ID NO: 13 - DNA, 31 bp, Artificial Sequence
  HIS-TNF reverse primer
gacctcgagtcacagggcaatgatcccaaag

SEQ ID NO: 14 - protein, 161 aa, Homo sapiens
MGSHHHHHHRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFGESGQVYFGIIAL

SEQ ID NO: 15 - protein, 338 aa, Yaba-like Disease Virus
MDKLLLFSTIVAVCNCITLKYNYTVTLKDDGLYDGVFYDHYNDQLVTKISYNHETRHGNVNFRADWFNISRSPHTPGNDYNFNFWYSLMKETLEEINKNDSTKTTSLSLITGCYETGLLFGSYGYVETANGPLARYHTGDKRFTKMTHKGFPKVGMLTVKNTLWKDVKAYLGGFEYMGCSLAILDYQKMAKGKIPKDTTPTVKVTGNELEDGNMTLECTVNSFYPPDVITKWIESEHFKGEYKYVNGRYYPEWGRKSNYEPGEPGFPWNIKKDKDANTYSLTDLVRTTSKMSSQPVCVVFHDTLEAQVYTCSEGCNGELYDHLYRKTEEGEGEEDEED

SEQ ID NO: 16 - protein, 338 aa, Tanapox Virus
MNKLILFSTIVAVCNCITLKYNYTVTLKDNGLYDGVFYDHYNDQLVTKISYNHETRHGNVNFRADWFNISRSPHTPGNDYNFNFWYSLMKETLEEINKNDSTKTTSLSLITGCYETGLLFGSYGYVETANGPLARYHTGDKRFTKMTHKGFPKVGMLTVKNTLWKDVKTYLGGFEYMGCSLAILDYQKMAKGEIPKDTTPTVKVTGNELEDGNMTLECSVNSFYPPDVITKWIESEHFKGEYKYVNGRYYPEWGRKSDYEPGEPGFPWNIKKDKDANTYSLTDLVRTTSKMSSQLVCVVFHDTLEAQVYTCSEGCNGELYDHLYRKTEEGEGEEDEED

SEQ ID NO: 17 - protein, 341 aa, Yaba monkey tumor virus
MNKLILSLLGFVATCNCITLRYNYTVTVKNGLYDGVFFDYYNDQLVTRISYNHETRHGNVNSRASWFDISKSPHTPGDDYHFNFWYPLMKDTLESINSNKNESDKCSSLSLILGCYETGSLFGSYGYVESSGGPLARYSTKDKKFLKMTDKGFPKVGMLTVHGPSWQTVKKYVGGFVYAGCLLAIFDYQKMAKNNIPSNVMPTVTVTGEELQDGNTTLKCNVKSFYPPDVMIKWIESKYFNGEYRYVNGREYPEWGRQSDYEPGEPGFPLHPKKDDGKTTYSLLDFGRTTSGLTSQLVCVVFHDTFESQVNTCSEGCEGKLYDHLYRKSEEGDEVVEDEED

SEQ ID NO: 18 - protein, 329 aa, Yaba-like Disease Virus
ITLKYNYTVTLKDDGLYDGVFYDHYNDQLVTKISYNHETRHGNVNFRADWFNISRSPHTPGNDYNFNFWYSLMKETLEEINKNDSTKTTSLSLITGCYETGLLFGSYGYVETANGPLARYHTGDKRFTKMTHKGFPKVGMLTVKNTLWKDVKAYLGGFEYMGCSLAILDYQKMAKGKIPKDTTPTVKVTGNELEDGNMTLECTVNSFYPPDVITKWIESEHFKGEYKYVNGRYYPEWGRKSNYEPGEPGFPWNIKKDKDANTYSLTDLVRTTSKMSSQPVCVVFHDTLEAQVYTCSEGCNGELYDHLYRKTEEGEGLEDIEGRHHHHHH